(19) United States
(12) Patent Application Publication
(10) Pub. No.: US 2026/0027220 A1
(43) Pub. Date: Jan. 29, 2026

(54) EPHA2 BCL-XL INHIBITOR ANTIBODY-DRUG CONJUGATES AND METHODS OF USE THEREOF

(71) Applicants: Novartis AG, Basel (CH); Les Laboratoires Servier, Suresnes Cedex (FR)

(72) Inventors: Joseph Anthony D'Alessio, Cambridge, MA (US); Tibor Novák, Budapest (HU); Jérôme-Benoit Starck, Rueil-Malmaison (FR); Zhuoliang Chen, Belmont, MA (US); Anthony Sotirios Malamas, Bedford, MA (US); Eric Andrew McNeill, Arlington, MA (US); Richard Vaughan Newcombe, Acton, MA (US); Bing Yu, Belmont, MA (US)

(21) Appl. No.: 18/866,144

(22) PCT Filed: May 19, 2023
(86) PCT No.: PCT/US2023/022937

§ 371 Date: Nov. 15, 2024

Related U.S. Application Data
(60) Provisional application No. 63/344,454, filed on May 20, 2022.

(51) Int. Cl. A61K 47/68; A61P 35/00; C07K 16/28; G01N 33/574

(52) U.S. Cl. CPC A61K 47/6803; A61K 47/6849; A61K 47/6889; A61P 35/00; C07K 16/2866; G01N 33/57492

(57) ABSTRACT

Anti-EphA2 antibody-drug conjugates that bind to human oncology targets are disclosed. The antibody-drug conjugates comprise a Bcl-xL inhibitor drug moiety and an anti-EphA2 antibody or antigen-binding fragment thereof that binds the antigen target, e.g., the antigen expressed on a tumor or other cancer cells. The disclosure further relates to methods and compositions for use in the treatment of cancers by administering the antibody-drug conjugates provided herein. Linker-drug conjugates comprising Bcl-xL inhibitor drug moiety and methods of making the same are also disclosed.

### RELATED APPLICATION

This application claims the benefit of the filing date, under 35 U.S.C. § 119(e), of U.S. Provisional Application No. 63/344,454, filed on May 20, 2022, the entire contents of which are incorporated here by reference.

### FIELD OF THE INVENTION

The present disclosure relates to antibody-drug conjugates (ADCs) comprising a Bcl-xL inhibitor and an anti-EphA2 antibody or antigen-binding fragment thereof that binds the antigen target, e.g., the antigen expressed on a tumor or other cancer cell. The disclosure further relates to methods and compositions useful in the treatment and/or diagnosis of cancers that express a target antigen and/or are amenable to treatment by modulating Bcl-xL expression and/or activity, as well as methods of making those compositions. Linker-drug conjugates comprising a Bcl-xL inhibitor drug moiety and methods of making same are also disclosed.

### BACKGROUND OF THE INVENTION

Apoptosis (programmed cell death) is an evolutionarily conserved pathway essential for tissue homeostasis, development and removal of damaged cells. Deregulation of apoptosis contributes to human diseases, including malignancies, neurodegenerative disorders, diseases of the immune system and autoimmune diseases (Hanahan and Weinberg, Cell. 2011 Mar. 4; 144 (5):646-74; Marsden and Strasser, Annu Rev Immunol. 2003; 21:71-105; Vaux and Flavell, Curr Opin Immunol. 2000 December; 12 (6):719-24). Evasion of apoptosis is recognized as a hallmark of cancer, participating in the development as well as the sustained expansion of tumors and the resistance to anti-cancer treatments (Hanahan and Weinberg, Cell. 2000 Jan. 7; 100 (1):57-70).

The Bcl-2 protein family comprises key regulators of cell survival which can suppress (e.g., Bcl-2, Bcl-xL, Mcl-1) or promote (e.g., Bad, Bax) apoptosis (Gross et al., Genes Dev. 1999 Aug. 1; 13 (15):1899-911, Youle and Strasser, Nat. Rev. Mol. Cell Biol. 2008 January; 9 (1):47-59).

In the face of stress stimuli, whether a cell survives or undergoes apoptosis is dependent on the extent of pairing between the Bcl-2 family members that promote cell death with family members that promote cell survival. For the most part, these interactions involve the docking of the Bcl-2 homology 3 (BH3) domain of proapoptotic family members into a groove on the surface of pro-survival members. The presence of Bcl-2 homology (BH) domain defines the membership of the Bcl-2 family, which is divided into three main groups depending upon the particular BH domains present within the protein. The prosurvival members such as Bcl-2, Bcl-xL, and Mcl-1 contain BH domains 1-4, whereas Bax and Bak, the proapoptotic effectors of mitochondrial outer membrane permeabilization during apoptosis, contain BH domains 1-3 (Youle and Strasser, Nat. Rev. Mol. Cell Biol. 2008 January; 9 (1):47-59).

Overexpression of the prosurvival members of the Bcl-2 family is a hallmark of cancer and it has been shown that these proteins play an important role in tumor development, maintenance and resistance to anticancer therapy (Czabotar et al., Nat. Rev. Mol. Cell Biol. 2014 January; 15 (1):49-63). Bcl-xL (also named BCL2L1, from BCL2-like 1) is frequently amplified in cancer (Beroukhim et al., Nature 2010 Feb. 18; 463 (7283):899-905) and it has been shown that its expression inversely correlates with sensitivity to more than 120 anti-cancer therapeutic molecules in a representative panel of cancer cell lines (NCI-60)(Amundson et al., Cancer Res. 2000 Nov. 1; 60 (21):6101-10).

In addition, several studies using transgenic knockout mouse models and transgenic overexpression of Bcl-2 family members highlighted the importance of these proteins in the diseases of the immune system and autoimmune diseases (for a review, see Merino et al., Apoptosis 2009 April; 14 (4):570-83. doi: 10.1007/s10495-008-0308-4.PMID: 19172396). Transgenic overexpression of Bcl-xL within the T-cell compartment resulted in resistance to apoptosis induced by glucocorticoid, g-radiation and CD3 crosslinking, suggesting that transgenic Bcl-xL overexpression can reduce apoptosis in resting and activated T-cells (Droin et al., Biochim Biophys Acta 2004 Mar. 1; 1644 (2-3):179-88. doi: 10.1016/j.bbamcr.2003.10.011.PMID: 14996502). In patient samples, persistent or high expression of antiapoptotic Bcl-2 family proteins has been observed (Pope et al., Nat Rev Immunol. 2002 July; 2 (7):527-35. doi: 10.1038/nri846.PMID: 12094227). In particular, T-cells isolated from the joints of rheumatoid arthritis patients exhibited increased Bcl-xL expression and were resistant to spontaneous apoptosis (Salmon et al., J Clin Invest. 1997 Feb. 1; 99 (3):439-46. doi: 10.1172/JCI119178.PMID: 9022077).

The findings indicated above motivated the discovery and development of a new class of drugs named BH3 mimetics. These molecules are able to disrupt the interaction between the proapoptotic and antiapoptotic members of the Bcl-2 family and are potent inducers of apoptosis. This new class of drugs includes inhibitors of Bcl-2, Bcl-xL, Bcl-w and Mcl-1. The first BH3 mimetics described were ABT-737 and ABT-263, targeting Bcl-2, Bcl-xL and Bcl-w (Park et al., J. Med. Chem. 2008 Nov. 13; 51 (21):6902-15; Roberts et al., J. Clin. Oncol. 2012 February 10; 30 (5):488-96). After that, selective inhibitors of Bcl-2 (ABT-199 and S55746-Souers et al., Nat Med. 2013 February; 19 (2):202-8; Casara et al., Oncotarget 2018 Apr. 13; 9 (28):20075-20088), Bcl-xL (A-1155463 and A-1331852-Tao et al., ACS Med Chem Lett. 2014 Aug. 26; 5 (10):1088-93; Leverson et al., Sci Transl Med. 2015 Mar. 18; 7 (279):279ra40) and Mcl-1 (A-1210477, S63845, S64315, AMG-176 and AZD-5991-Leverson et al., Cell Death Dis. 2015 Jan. 15; 6: e1590.; Kotschy et al., Nature 2016, 538, 477-482; Maragno et al., AACR 2019, Poster #4482; Kotschy et al., WO 2015/097123; Caenepeel et al., Cancer Discov. 2018 December; 8 (12):1582-1597; Tron et al., Nat. Commun. 2018 Dec. 17; 9 (1):5341) were also discovered. The selective Bcl-2 inhibitor ABT-199 is now approved for the treatment of patients with CLL and AML in combination therapy, while the other inhibitors are still under pre-clinical or clinical development. In pre-clinical models, ABT-263 has shown activity in several hematological malignancies and solid tumors (Shoemaker et al., Clin. Cancer Res. 2008 Jun. 1; 14 (11):3268-77; Ackler et al., Cancer Chemother. Pharmacol. 2010 October; 66 (5):869-80; Chen et al., Mol. Cancer Ther. 2011 December; 10 (12):2340-9). In clinical studies, ABT-263 exhibited objective antitumor activity in lymphoid malignancies (Wilson et al., Lancet Oncol. 2010 December; 11 (12):1149-59; Roberts et al., J. Clin. Oncol. 2012 Feb. 10; 30 (5):488-96) and its activity is being investigated in combination with several therapies in solid tumors. The selective Bcl-xL inhibitors, A-1155463 or A-1331852, exhibited in vivo activity in pre-clinical models of T-ALL (T-cell Acute Lymphoblastic Leukemia) and different types of solid tumors (Tao et al., ACS Med. Chem. Lett. 2014 Aug. 26; 5 (10):1088-93; Leverson et al., Sci. Transl. Med. 2015 Mar. 18; 7 (279):279ra40). The use of BH3 mimetics has also shown benefit in pre-clinical models of diseases of the immune system and autoimmune diseases. Treatment with ABT-737 (Bcl-2, Bcl-xL, and Bcl-w inhibitor) resulted in potent inhibition of lymphocyte proliferation in vitro. Importantly, mice treated with ABT-737 in animal models of arthritis and lupus showed a significant decrease in disease severity (Bardwell et al., J Clin Invest. 1997 Feb. 1; 99 (3):439-46. doi: 10.1172/JCI119178.PMID: 9022077). In addition, it has been shown that ABT-737 prevented allogeneic T-cell activation, proliferation, and cytotoxicity in vitro and inhibited allogeneic T- and B-cell responses after skin transplantation with high selectivity for lymphoid cells (Cippa et al., Transpl Int. 2011 July; 24 (7):722-32. doi: 10.1111/j.1432-2277.2011.01272.x. Epub 2011 May 25.PMID: 21615547). Therefore, therapeutically targeting Bcl-xL or proteins upstream and/or downstream of it in an apoptotic signaling pathway represent a highly attractive approach for the development of novel therapies in oncology and in the field of immune and autoimmune diseases.

EphA2 receptor belongs to the ephrin receptor subfamily of receptor tyrosine kinases. It has been shown that EphA2 is highly produced in tumor tissues; while present at relatively low levels in most normal adult tissues. EphA2 dysregulation has been associated with various pathological processes, especially cancer. For certain types of cancers EphA2 is linked with poor prognosis and decreased patient survival. Thus, EphA2 receptor is an attractive target for antibody drug conjugates.

### SUMMARY OF THE INVENTION

In some embodiments, the present disclosure provides, in part, novel antibody-drug conjugate (ADC) compounds with biological activity against cancer cells. The compounds may slow, inhibit, and/or reverse tumor growth in mammals, and/or may be useful for treating human cancer patients. The present disclosure more specifically relates, in some embodiments, to ADC compounds that are capable of binding and killing cancer cells. In some embodiments, the ADC compounds disclosed herein comprise a linker that attaches a Bcl-xL inhibitor to a full-length anti-EphA2 antibody or an antigen-binding fragment. In some embodiments, the ADC compounds are also capable of internalizing into a target cell after binding.

In some embodiments, ADC compounds may be represented by Formula (1):

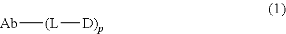

wherein Ab is an anti-EphA2 antibody or an antigen-binding fragment thereof;
D is a Bcl-xL inhibitor;
L is a linker that covalently attaches Ab to D; and
p is an integer from 1 to 16. In some embodiments, Ab is an antibody or an antigen-binding fragment thereof that targets a cancer cell.

In some embodiments, for ADC compounds of Formula (1), D comprises a Bcl-xL inhibitor compound of Formula (I′) or Formula (II′) covalently attached to the linker L:

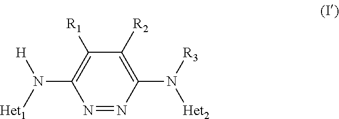

or an enantiomer, a diastereoisomer, and/or a pharmaceutically acceptable salt of any one of the foregoing, wherein:
R₁ and R₂ independently of one another represent a group selected from the group consisting of: hydrogen; a linear or branched C₁-C₆alkyl optionally substituted by a hydroxyl or a C₁-C₆alkoxy group; a C₃-C₆cycloalkyl; a trifluoromethyl; and a linear or branched C₁-C₆alkylene-heterocycloalkyl wherein the heterocycloalkyl group is optionally substituted by a linear or branched C₁-C₆alkyl group;
or R₁ and R₂ form with the carbon atoms carrying them a C₃-C₆cycloalkylene group,
R₃ represents a group selected from the group consisting of: hydrogen; a C₃-C₆cycloalkyl; a linear or branched C₁-C₆alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:

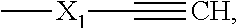

Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group:

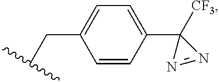 [SMILES: *Cc1ccc(C2(C(F)(F)F)N=N2)cc1]

or Rₐ and R_{b} form with the nitrogen atom carrying them a cycle B₁;
or Rₐ, R_{b} and R_{c} form with the nitrogen atom carrying them a bridged C₃-C₈hetero cycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈hetero cycloalkyl,
Het₁ represents a group selected from the group consisting of:

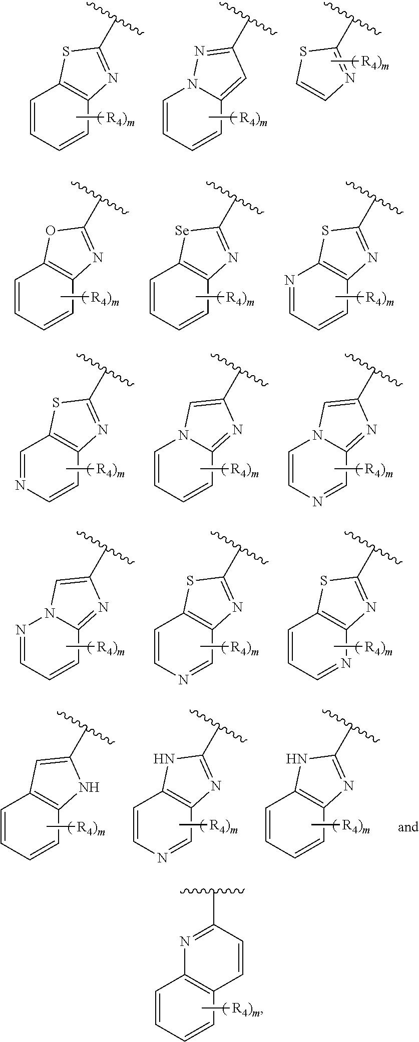

Het₂ represents a group selected from the group consisting of:

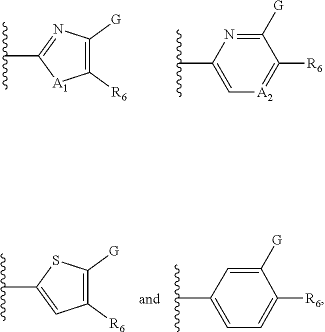

A₁ is —NH—, —N(C₁-C₃alkyl), O, S or Se,
A₂ is N, CH or C(R₅),
G is selected from the group consisting of:
   —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, a halogen, —NO₂, and —CN, in which:
   R_{G1} and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl, a C₃-C₆cycloalkyl, phenyl and —(CH₂)₁₋₄-phenyl;
   R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₈heterocycloalkyl; or in the alternative, G is selected from the group consisting of:

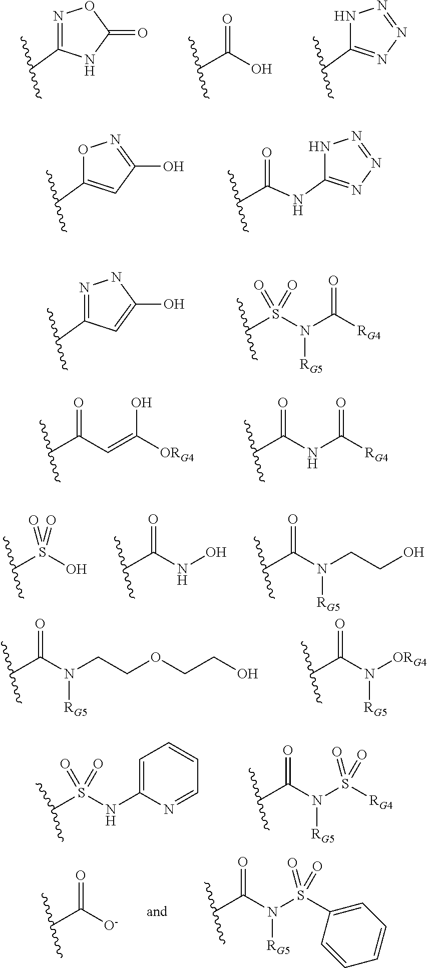

wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆alkynyl and a C₃-C₆cycloalkyl, and
R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆ represents a group selected from the group consisting of:
hydrogen;
a linear or branched —C₁-C₆alkylene-R₈ group;
a —C₂-C₆alkenyl;
—X₂—O—R₇;

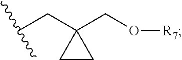

—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₆) cycloalkylene-R₈;

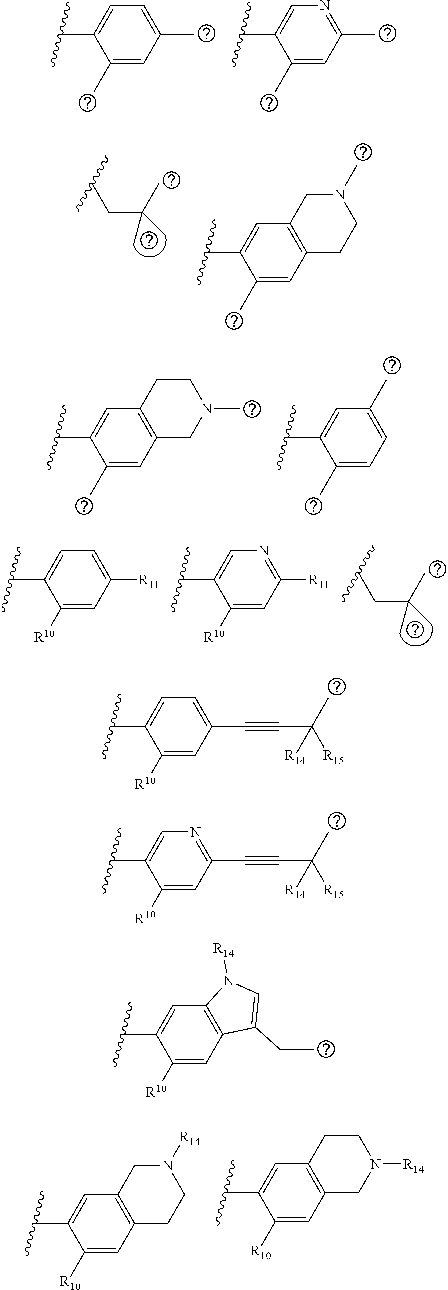

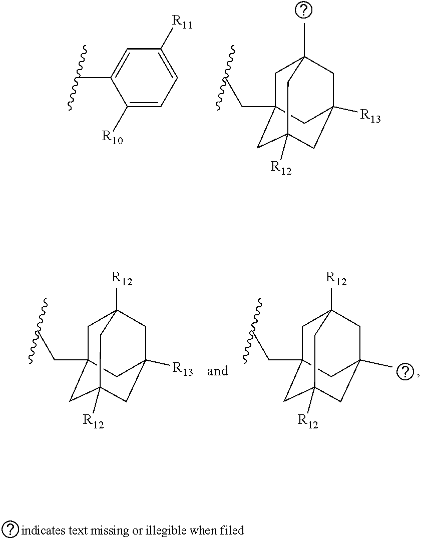

wherein Cy represents a C₃-C₈cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}; —X′₂NR′ₐR′_{b}; —NR′_{c}—X′₂—N₃ and

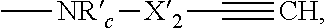

R₉ represents a group selected from the group consisting of a linear or branched C₁-C₆alkyl, trifluoromethyl, hydroxyl, halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a C₁-C₃alkylene-R₈, a —O—C₁-C₃alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy group; and the group:

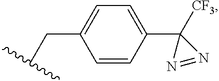 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₈ hetero cycloalkyl,
R′_{c}, R′_{d}, R′ₑ, R′_{f}, independently of one another, represents a hydrogen or a linear or branched C₁-C₆alkyl group,
or R′ₐ and R′ₑ form with the nitrogen atom carrying them a cycle B₄,
or R′_{d}, R′ₑ and R′_{f} form with the nitrogen atom carrying them a bridged C₃-C_{8 D}heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
   wherein one of the R₃ and R₈ groups, if present, is covalently attached to the linker, and wherein the valency of an atom is not exceeded by virtue of one or more substituents bonded thereto; or

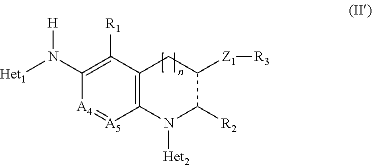

or an enantiomer, a diastereoisomer, and/or a pharmaceutically acceptable salt of any one of the foregoing, wherein:
n=0, 1 or 2,
[symbol] represents a single or a double bond,
A₄ and A₅ independently of one another represent a carbon or a nitrogen atom,
Z₁ represents a bond, —N(R)—, or —O—, wherein R represents a hydrogen or a linear or branched C₁-C₆alkyl,
R₁ represents a group selected from the group consisting of: hydrogen; a linear or branched C₁-C₆alkyl optionally substituted by a hydroxyl or a C₁-C₆alkoxy group; a C₃-C₆cycloalkyl; trifluoromethyl; and a linear or branched C₁-C₆alkylene-heterocycloalkyl wherein the heterocycloalkyl group is optionally substituted by a linear or branched C₁-C₆alkyl group;
R₂ represents a hydrogen or a methyl;
R₃ represents a group selected from the group consisting of: hydrogen; a linear or branched C₁-C₄alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:

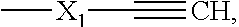

Rₐ and R_{b} independently of one another represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl groups; a C₁-C₆alkylene-SO₂OH; a C₁-C₆alkylene-SO₂O; a C₁-C₆alkylene-COOH; a C₁-C₆alkylene-PO(OH)₂; a C₁-C₆alkylene-NR_{d}Rₑ; a C₁-C₆alkylene-N⁺R_{d}RₑR_{f}; a C₁-C₆alkylene-phenyl wherein the phenyl may be substituted by a C₁-C₆alkoxy group; and the group:

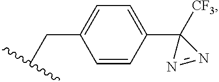 [SMILES: *Cc1ccc(C2(C(F)(F)F)N=N2)cc1]

or Rₐ and R_{b} form with the nitrogen atom carrying them a cycle B₁;
or Rₐ, R_{b} and R_{c} form with the nitrogen atom carrying them a bridged C₃-C₈ heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈ heterocycloalkyl,
Het₁ represents a group selected from the group consisting of:

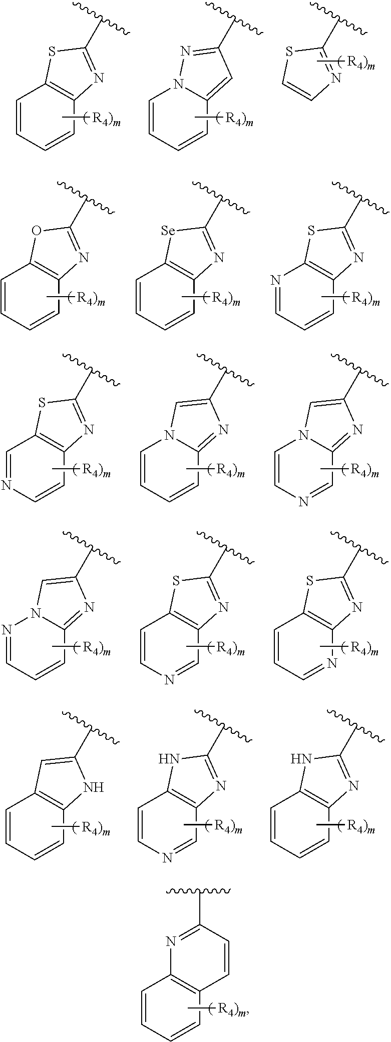

Het₂ represents a group selected from the group consisting of:

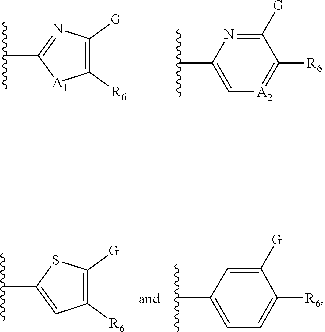

A₁ is —NH—, —N(C₁-C₆alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, —C₁-C₆alkyl optionally substituted by a hydroxyl group, halogen, —NO₂, and —CN, in which:
R_{G1} and R_{G2} at each occurrence are each independently selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆alkenyl, a C₂-C₆ alkynyl, a C₃-C₆cycloalkyl, phenyl and —(CH₂)₁₋₄-phenyl;
R_{G3} is selected from the group consisting of a C₁-C₆alkyl optionally substituted by 1 to 3 halogen atoms, a C₂-C₆alkenyl, a C₂-C₆alkynyl, a C₃-C₆cycloalkyl, phenyl and —(CH₂)₁₋₄-phenyl; or R_{G1} and R_{G2}, together with the atom to which each is attached are combined to form a C₃-C₈heterocycloalkyl; or in the alternative, G is selected from the group consisting of:

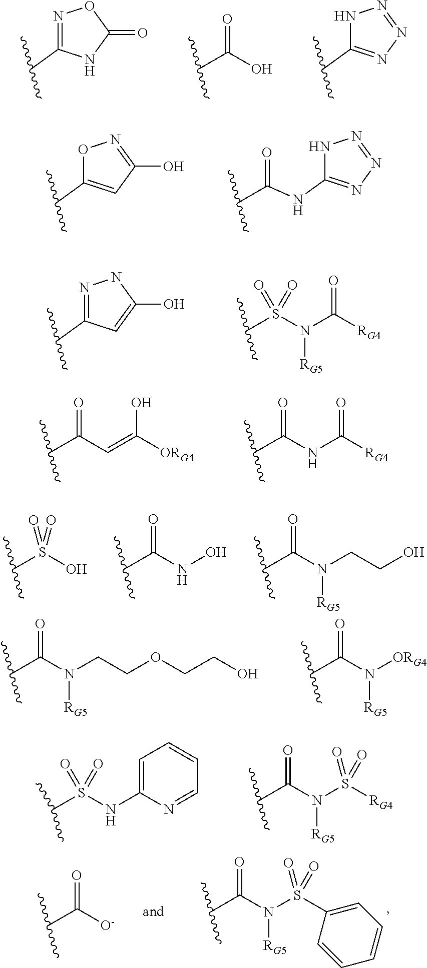

wherein R_{G4} is selected from the group consisting of hydrogen, a C₁-C₆alkyl optionally substituted by 1 to 3 halogen atoms, a C₁-C₆alkyl substituted by a hydroxyl, a C₁-C₆alkyl substituted by a C₁-C₆alkoxy group, a C₂-C₆ alkenyl, a C₂-C₆alkynyl and a C₃-C₆cycloalkyl, and R_{G5} represents a hydrogen atom or a C₁-C₆alkyl group optionally substituted by 1 to 3 halogen atoms,
R₄ represents a hydrogen, fluorine, chlorine or bromine atom, a methyl, a hydroxyl or a methoxy group,
R₅ represents a group selected from the group consisting of: a C₁-C₆alkyl optionally substituted by 1 to 3 halogen atoms; a C₂-C₆alkenyl; a C₂-C₆alkynyl; a halogen; and —CN,
R₆ represents a group selected from the group consisting of:
hydrogen;
a linear or branched-C₁-C₆alkylene-R₈ group;
a —C₂-C₆alkenyl;
—X₂—O—R₇;

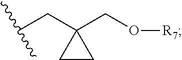

—X₂—NSO₂—R₇;
—C═C(R₉)—Y₁—O—R₇;
a C₃-C₆cycloalkyl;
a C₃-C₆heterocycloalkyl optionally substituted by a hydroxyl group;
a C₃-C₆cycloalkylene-Y₂—R₇;
a C₃-C₆heterocycloalkylene-Y₂—R₇ group, and
a heteroarylene-R₇ group optionally substituted by a linear or branched C₁-C₆alkyl group,
R₇ represents a group selected from the group consisting of: a linear or branched C₁-C₆alkyl group; a (C₃-C₅) cycloalkylene-R₈;

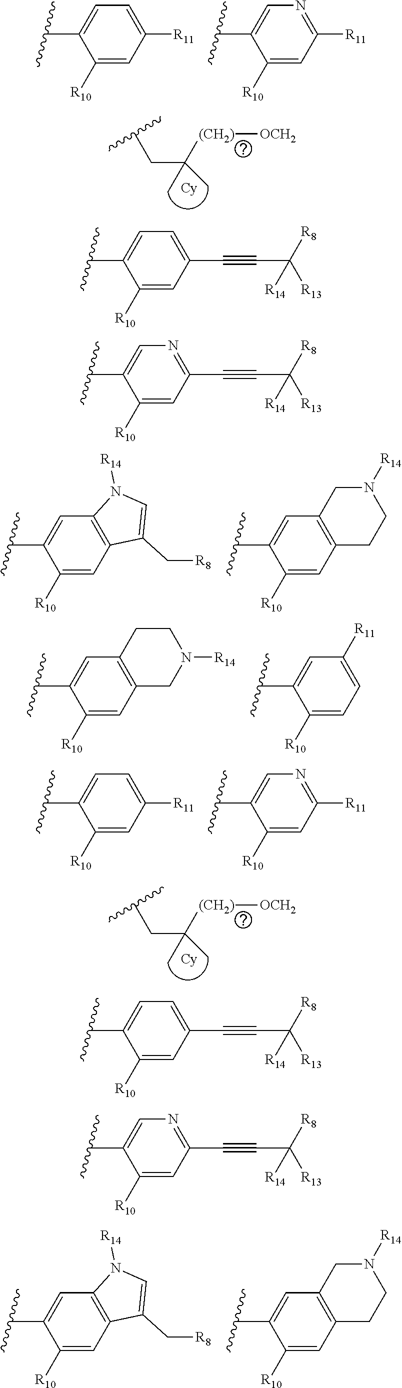

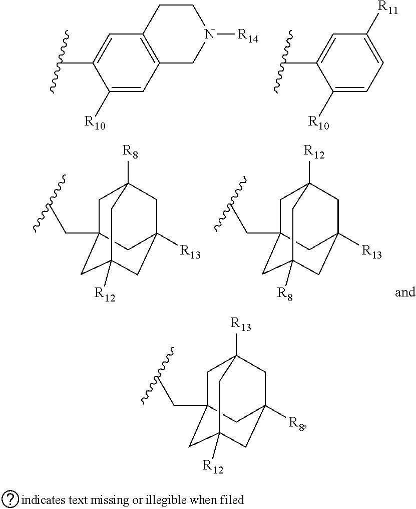

wherein Cy represents a C₃-C₆cycloalkyl,
R₈ represents a group selected from the group consisting of: hydrogen; a linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}OR′_{c}; —O—X′₂—NR′ₐR′_{b}, —X′₂—NR′ₐR′_{b}, —NR′_{c}—X′₂—N₃ and:

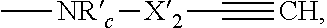

R₉ represents a group selected from the group consisting of a linear or branched C₁-C₆alkyl, trifluoromethyl, hydroxyl, a halogen, and a C₁-C₆alkoxy,
R₁₀ represents a group selected from the group consisting of hydrogen, fluorine, chlorine, bromine, —CF₃ and methyl,
R₁₁ represents a group selected from the group consisting of hydrogen, a halogen, a C₁-C₃alkylene-R₈, a —O—C₁-C₆alkylene-R₈, —CO—NRₕRᵢ and a —CH═CH—C₁-C₄alkylene-NRₕRᵢ, —CH═CH—CHO, a C₃-C₈cycloalkylene-CH₂—R₈, and a C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ represents a linear or branched C₁-C₄alkylene group optionally substituted by one or two groups selected from the group consisting of trifluoromethyl, hydroxyl, a halogen, and a C₁-C₆alkoxy,
X₂ represents a linear or branched C₁-C₆alkylene group optionally substituted by one or two groups selected from the group consisting of trifluoromethyl, hydroxyl, a halogen, and a C₁-C₆alkoxy,
X′₂ represents a linear or branched C₁-C₆alkylene,
R′ₐ and R′_{b} independently of one another, represent a group selected from the group consisting of: hydrogen; a heterocycloalkyl; —SO₂-phenyl wherein the phenyl may be substituted by a linear or branched C₁-C₆alkyl; a linear or branched C₁-C₆alkyl optionally substituted by one or two hydroxyl or C₁-C₆alkoxy groups; a C₁-C₆alkylene-SO₂OH; a C₁-C₆alkylene-SO₂O; a C₁-C₆alkylene-COOH; a C₁-C₆alkylene-PO(OH)₂; a C₁-C₆alkylene-NR′_{d}R′ₑ; a C₁-C₆alkylene-N⁺R′_{d}R′ₑR′_{f}; a C₁-C₆alkylene-O—C₁-C₆alkylene-OH; a C₁-C₆alkylene-phenyl wherein the phenyl may be substituted by a hydroxyl or a C₁-C₆alkoxy
group; and the group:

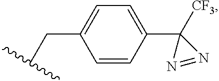 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₈ heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈ heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, or —NH—SO₂—,
m=0, 1 or 2,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from the group consisting of: fluorine, bromine, chlorine, a linear or branched C₁-C₆alkyl, hydroxyl, —NH₂, oxo and piperidinyl,
wherein one of the R₃, R₈ and G groups, if present, is covalently attached to the linker, and wherein the valency of an atom is not exceeded by virtue of one or more substituents bonded thereto.

In some embodiments, for ADC compounds of Formula (I), D comprises a Bcl-xL inhibitor compound of Formula (I) or Formula (II) covalently attached to the linker L:

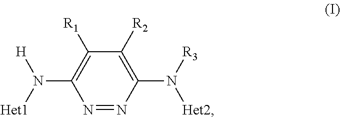

or an enantiomer, a diastereoisomer, and/or an addition salt thereof with a pharmaceutically acceptable acid or base (i.e., a pharmaceutically acceptable salt) of any one of the foregoing, wherein:
R₁ and R₂ independently of one another represent a group selected from: hydrogen; linear or branched C₁-C₆alkyl optionally substituted by a hydroxyl or a
   C₁-C₆alkoxy group; C₃-C₆cycloalkyl; trifluoromethyl; linear or branched
   C₁-C₆alkylene-heterocycloalkyl wherein the heterocycloalkyl group is optionally substituted by a linear or branched C₁-C₆alkyl group;
or R₁ and R₂ form with the carbon atoms carrying them a C₃-C₆cycloalkylene group,
R₃ represents a group selected from: hydrogen; C₃-C₆cycloalkyl; linear or branched C₁-C₆alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:

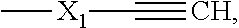

Rₐ and R_{b} independently of one another represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl groups; C₁-C₆alkylene-SO₂OH; C₁-C₆alkylene-SO₂O; C₁-C₆alkylene-COOH; C₁-C₆alkylene-PO(OH)₂; C₁-C₆alkylene-NR_{d}Rₑ; C₁-C₆alkylene-N⁺R_{d}RₑR_{f}; C₁-C₆alkylene-phenyl wherein the phenyl may be substituted by a C₁-C₆alkoxy group;
the group:

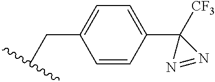 [SMILES: *Cc1ccc(C2(C(F)(F)F)N=N2)cc1]

or Rₐ and R_{b} form with the nitrogen atom carrying them a cycle B₁;
or Rₐ, R_{b} and R_{c} form with the nitrogen atom carrying them a bridged C₃-C₈heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈heterocycloalkyl,
Het₁ represents a group selected from:

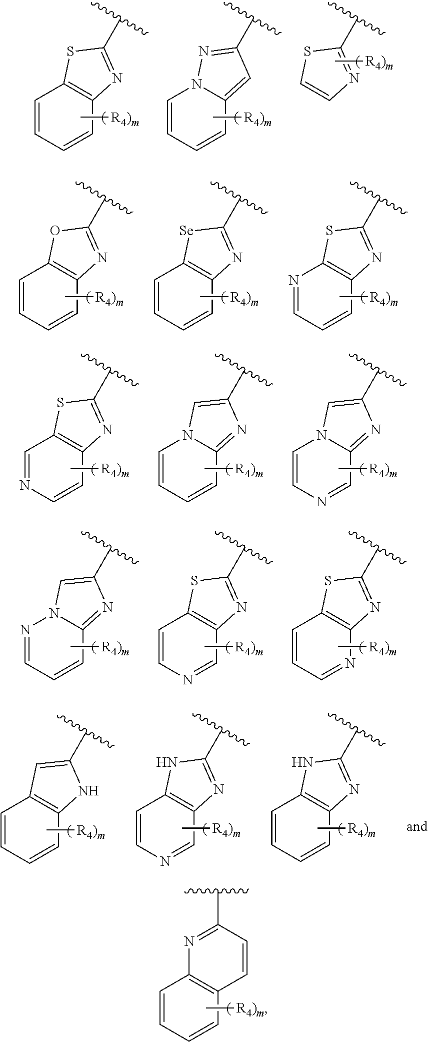

Het₂ represents a group selected from:

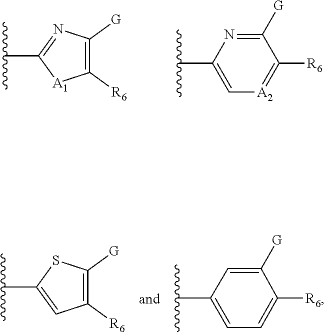

A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, C₁-C₆alkyl optionally substituted by a hydroxyl group, halogen, —NO₂, and —CN, in which:
R_{G1} and R_{G2} at each occurrence are each independently selected from the group consisting of hydrogen, C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl;
R_{G3} is selected from the group consisting of C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl; or
R_{G1} and R_{G2}, together with the atom to which each is attached are combined to form a C₃-C₈heterocycloalkyl; or in the alternative, G is selected from the group consisting of:

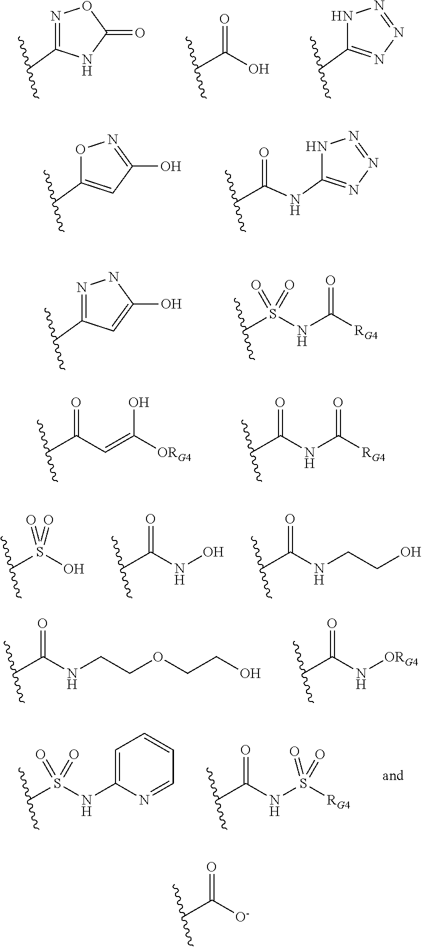

wherein R_{G4} is selected from hydrogen, C₁-C₆alkyl optionally substituted by 1 to 3 halogen atoms, C₂-C₆alkenyl, C₂-C₆alkynyl and C₃-C₆cycloalkyl,
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; C₂-C₆alkenyl; C₂-C₆alkynyl; halogen or —CN,
R₆ represents a group selected from:
hydrogen;
—C₂-C₆alkenyl;
—X₂—O—R₇;

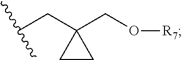

—X₂—NSO₂—R₇;
—C═C(R₉)—Y₁—O—R₇;
C₃-C₆cycloalkyl;
C₃-C₆heterocycloalkyl optionally substituted by a hydroxyl group;
C₃-C₆cycloalkylene-Y₂—R₇;
C₃-C₆heterocycloalkylene-Y₂—R₇ group,
an heteroarylene-R₇ group optionally substituted by a linear or branched C₁-C₆alkyl group,
R₇ represents a group selected from: linear or branched C₁-C₆alkyl group; (C₃-C₆) cycloalkylene-R₈; or:

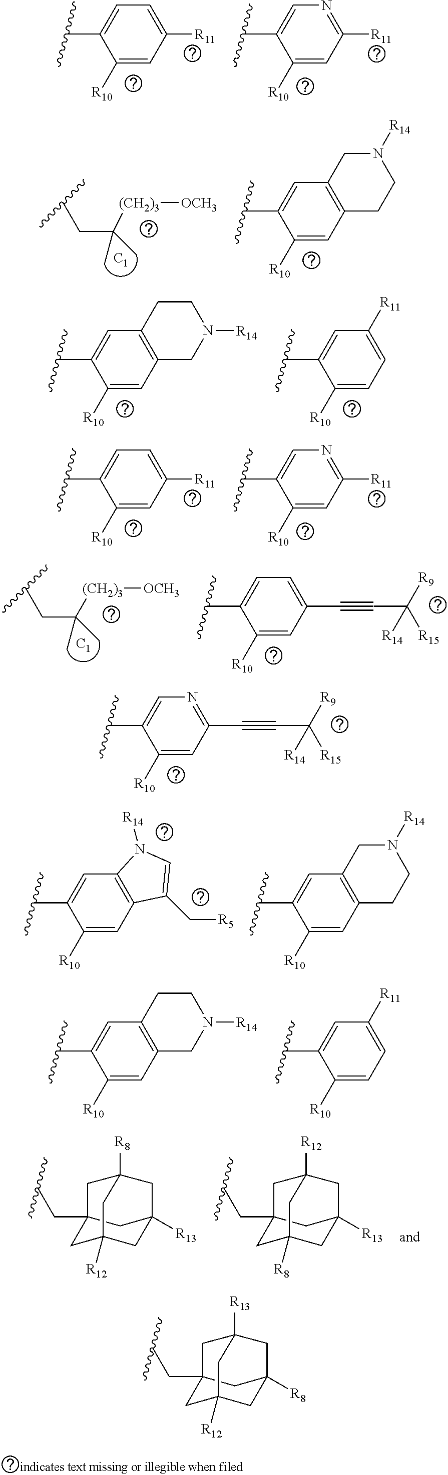

wherein Cy represents a C₃-C₈cycloalkyl,
R₈ represents a group selected from: hydrogen; linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and:

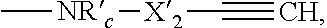

R₉ represents a group selected from linear or branched C₁-C₆alkyl, trifluoromethyl, hydroxyl, halogen, C₁-C₆alkoxy,
R₁₀ represents a group selected from hydrogen, fluorine, chlorine, bromine, —CF₃ and methyl,
R₁₁ represents a group selected from hydrogen, C₁-C₃alkylene-R₈, —O—C₁-C₆alkylene-R₈, —CO—NRₕRᵢ and —CH═CH—C₁-C₄alkylene-NRₕRᵢ, —CH═CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a
   a cyclohexyl,
Rₕ and Rᵢ, independently of one another, represent a hydrogen or a linear or branched C₁-C₆alkyl group,

X₁ and X₂ independently of one another, represent a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O; C₁-C₆alkylene-COOH; C₁-C₆alkylene-PO(OH)₂; C₁-C₆alkylene-NR′_{d}R′ₑ; C₁-C₆alkylene-N⁺R′_{d}R′ₑR′_{f}; C₁-C₆alkylene-O—C₁-C₆alkylene-OH; C₁-C₆alkylene-phenyl wherein the phenyl may be substituted by a hydroxyl or a C₁-C₆alkoxy group;
the group:

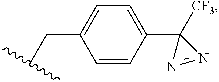 [SMILES: *Cc1ccc(C2(C(F)(F)F)N=N2)cc1]

or R′ₐ and R′_{b} form with the nitrogen atom carrying them a
cycle B₃,
or R′ₐ, R′_{b} and R′_{c} form with the nitrogen atom carrying them a bridged
C₃-C₈heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, —NH—SO₂—,
m=0, 1 or 2,
p=1, 2, 3 or 4,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl,
wherein one of the R₃ and R₈ groups, if present, is covalently attached to the linker, and wherein the valency of an atom is not exceeded by virtue of one or more substituents bonded thereto; or

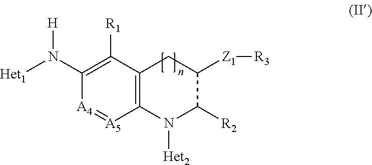

or an enantiomer, a diastereoisomer, and/or an addition salt thereof with a pharmaceutically acceptable acid or base (i.e., a pharmaceutically acceptable salt) of the foregoing, wherein: n=0, 1 or 2,
[symbol] represents a single or a double bond.
A₄ and A₅ independently of one another represent a carbon or a nitrogen atom,
Z₁ represents a bond, —N(R)—, or —O—, wherein R represents a hydrogen or a linear or branched C₁-C₆alkyl,
R₁ represents a group selected from: hydrogen; linear or branched C₁-C₆alkyl optionally substituted by a hydroxyl or a C₁-C₆alkoxy group; C₃-C₆cycloalkyl; trifluoromethyl; linear or branched C₁-C₆alkylene-heterocycloalkyl wherein the heterocycloalkyl group is optionally substituted by a linear or branched C₁-C₆alkyl group;
R₂ represents a hydrogen or a methyl;
R₃ represents a group selected from: hydrogen; linear or branched C₁-C₄alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and:

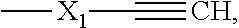

Rₐ and R_{b} independently of one another represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl groups; C₁-C₆alkylene-SO₂OH; C₁-C₆alkylene-SO₂O; C₁-C₆alkylene-COOH; C₁-C₆alkylene-PO(OH)₂; C₁-C₆alkylene-NR_{d}Rₑ; C₁-C₆alkylene-N⁺R_{d}RₑR_{f}; C₁-C₆alkylene-phenyl wherein the phenyl may be substituted by a C₁-C₆alkoxy group;
the group:

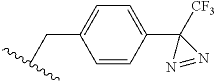 [SMILES: *Cc1ccc(C2(C(F)(F)F)N=N2)cc1]

or Rₐ and R_{b} form with the nitrogen atom carrying them a cycle B₁;
or Rₐ, R_{b} and R_{c} form with the nitrogen atom carrying them a bridged C₃-C₈heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈heterocycloalkyl,
Het₁ represents a group selected from:

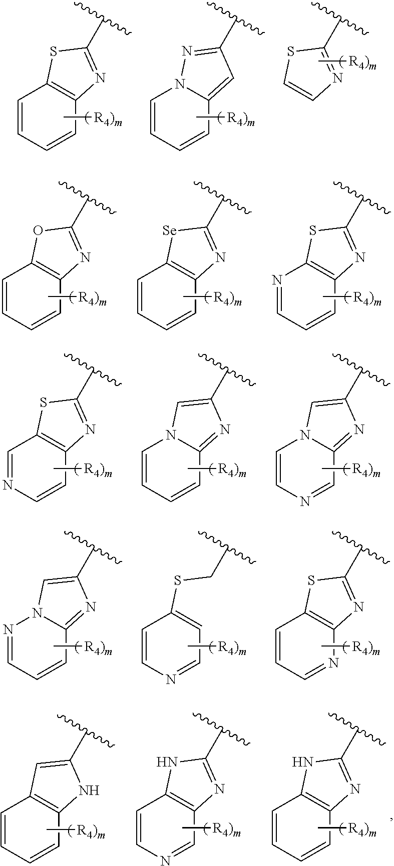

Het₂ represents a group selected from:

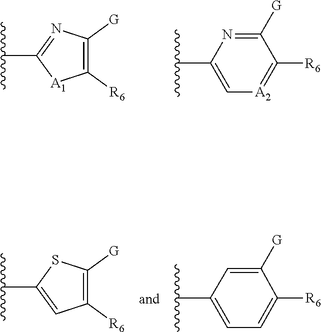

A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, C₁-C₆alkyl optionally substituted by a hydroxyl group, halogen, —NO₂, and —CN, in which:
   R_{G1} and R_{G2} at each occurrence are each independently selected from the group consisting of hydrogen, C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl;
   R_{G3} is selected from the group consisting of C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl; or
R_{G1} and R_{G2}, together with the atom to which each is attached are combined to form a C₃-C₈heterocycloalkyl; or in the alternative, G is selected from the group consisting of:

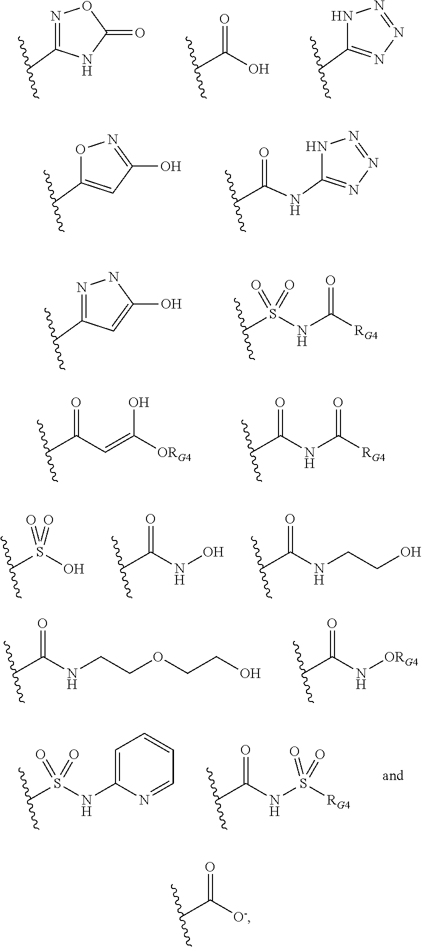

wherein R_{G4} is selected from hydrogen, C₁-C₆alkyl optionally substituted by 1 to 3 halogen atoms, C₂-C₆alkenyl, C₂-C₆alkynyl and C₃-C₆cycloalkyl,
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; C₂-C₆alkenyl; C₂-C₆alkynyl; halogen or —CN,
R₆ represents a group selected from:
hydrogen;
—C₂-C₆alkenyl;
—X₂—O—R₇;

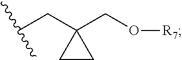

—X₂—NSO₂—R₇;
—C═C(R₉)—Y₁—O—R₇;
C₃-C₆cycloalkyl;
C₃-C₈heterocycloalkyl optionally substituted by a hydroxyl group;
C₃-C₆cycloalkylene-Y₂—R₇;
C₃-C₆heterocycloalkylene-Y₂—R₇ group,
an heteroarylene-R₇ group optionally substituted by a linear or branched C₁-C₆alkyl group,
R₇ represents a group selected from: linear or branched C₁-C₆alkyl group; (C₃-C₈) cycloalkylene-R₈; or:

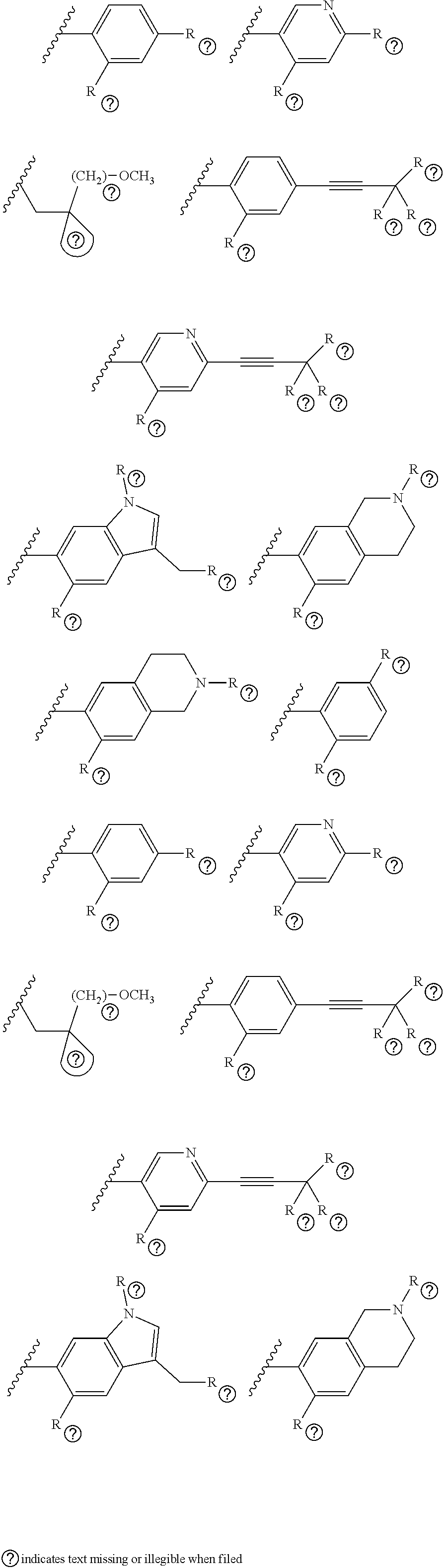

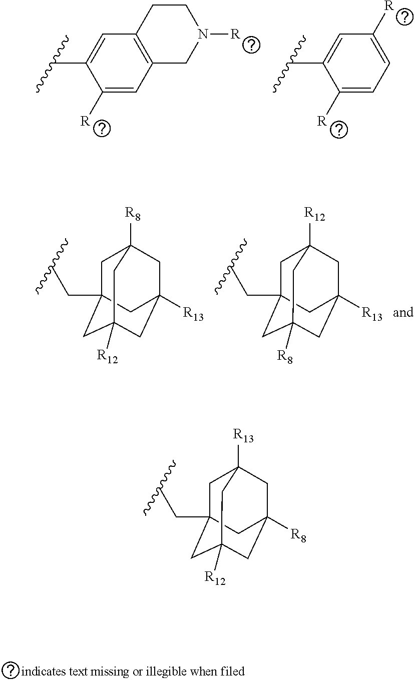

wherein Cy represents a C₃-C₈cycloalkyl,
   R₈ represents a group selected from: hydrogen; linear or branched C₁-C₆alkyl, —NR′ₐR'_{b}; —NR′ₐ—CO—OR′_{c}; —NR′ₐ—CO—R′_{c}; —N⁺R′ₐR′_{b}R′_{c}; —O—R′_{c}; —NH—X₂—N⁺R′ₐR′_{b}R′_{c}; —O—X′₂—NR′ₐR′_{b}, —X₂—NR′ₐR′_{b}, —NR′_{c}X₂—N₃ and:

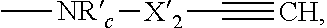

R₉ represents a group selected from linear or branched C₁-C₆alkyl, trifluoromethyl, hydroxyl, halogen, C₁-C₆alkoxy,
R₁₀ represents a group selected from hydrogen, fluorine, chlorine, bromine, —CF₃ and methyl,
R₁₁ represents a group selected from hydrogen, halogen, C₁-C₆alkylene-R₈, —O—C₁-C₃alkylene-R₈, —CO—NRₕRᵢ and —CH═CH—C₁-C₄alkylene-NRₕRᵢ, —CH═CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,

Rₕ and Rᵢ, independently of one another, represent a hydrogen or a linear or branched C₁-C₆alkyl group,
X₁ represents a linear or branched C₁-C₄alkylene group optionally substituted by one or two groups selected from trifluoromethyl, hydroxyl, halogen, C₁-C₆alkoxy,
X₂ represents a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O; C₁-C₆alkylene-COOH; C₁-C₆alkylene-PO(OH)₂; C₁-C₆alkylene-NR′_{d}R′ₑ;
   C₁-C₆alkylene-N⁺R′_{d}R′ₑR′_{f}; C₁-C₆alkylene-O—C₁-C₆alkylene-OH; C₁-C₆alkylene-phenyl wherein the phenyl may be substituted by a hydroxyl or a C₁-C₆alkoxy group;
the group:

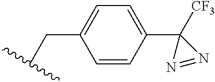 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₈heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, —NH—SO₂—,
m=0, 1 or 2,
p=1, 2, 3 or 4,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl,
wherein one of the R₃ and R₈ groups, if present, is covalently attached to the linker, and wherein the valency of an atom is not exceeded by virtue of one or more substituents bonded thereto.

In some embodiments, for Formula (I) or Formula (II), G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN, in which:
   R_{G1} and R_{G2} at each occurrence are each independently selected from the group consisting of hydrogen, C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl;
   R_{G3} is selected from the group consisting of C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl; or
R_{G1} and R_{G2}, together with the atom to which each is attached are combined to form a C₃-C₈heterocycloalkyl; or in the alternative, G is selected from the group consisting of:

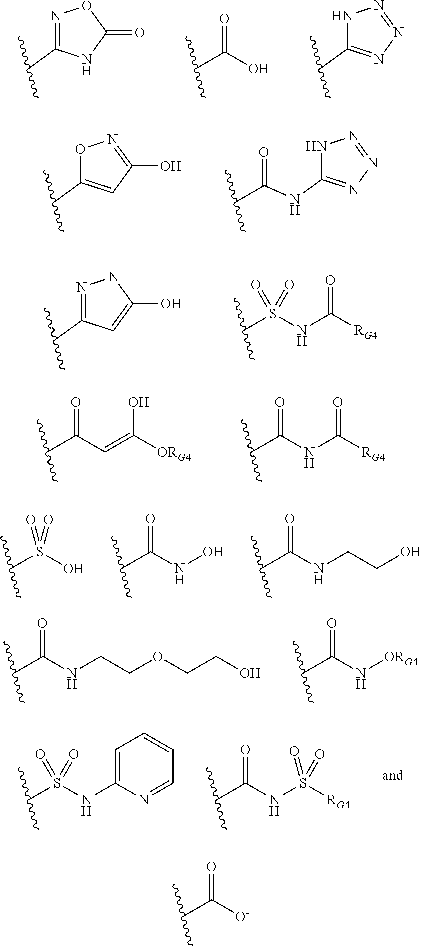

wherein R_{G4} is selected from C₁-C₆alkyl optionally substituted by 1 to 3 halogen atoms, C₂-C₆alkenyl, C₂-C₆alkynyl and C₃-C₆cycloalkyl.

In some embodiments, p is an integer from 1 to 8. In some embodiments, p is an integer from 1 to 5. In some embodiments, p is an integer from 2 to 4. In some embodiments, p is 2. In some embodiments, p is 4. In some embodiments, p is determined by liquid chromatography-mass spectrometry (LC-MS).

In some embodiments, the linker (L) comprises an attachment group, at least one spacer group, and at least one cleavable group. In some cases, the cleavable group comprises a pyrophosphate group and/or a self-immolative group. In specific embodiments, L comprises an attachment group; at least one bridging spacer group; and at least one cleavable group comprising a pyrophosphate group and/or a self-immolative group.

In some embodiments, the antibody-drug conjugate comprises a linker-drug (or “linker-payload”) moiety-(L-D) is of the formula (A):

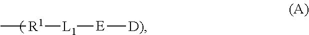

wherein R¹ is an attachment group, L₁ is a bridging spacer group, and E is a cleavable group.

In some embodiments, the cleavable group comprises a pyrophosphate group. In some embodiments, the cleavable group comprises:

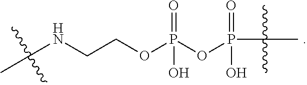 [SMILES: *NCCOP(=O)(O)OP(*)(=O)O]

In some embodiments, the bridging spacer group comprises a polyoxyethylene (PEG) group. In some cases, the PEG group may be selected from PEG1, PEG2, PEG3, PEG4, PEG5, PEG6, PEG7, PEG8, PEG9, PEG10, PEG11, PEG12, PEG13, PEG14, and PEG15. In some embodiments, the bridging spacer group may comprise: —CO—CH₂—CH₂-PEG12-. In other embodiments, the bridging spacer group comprises a butanoyl, pentanoyl, hexanoyl, heptanoyl, or octanoyl group. In some embodiments, the bridging spacer group comprises a hexanoyl group.

In some embodiments the attachment group is formed from at least one reactive group selected from a maleimide group, thiol group, cyclooctyne group, and an azido group. For example, maleimide group may have the structure:

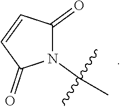 [SMILES: *N1C(=O)C=CC1=O]

The azido group may have the structure: —N═N⁺═N.

The cyclooctyne group may have the structure:

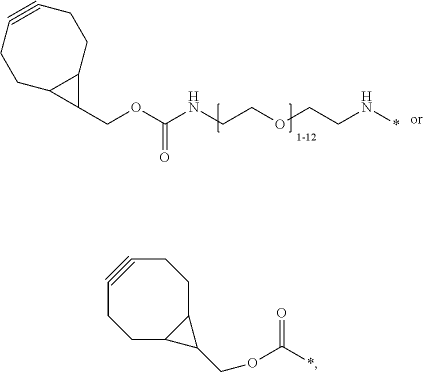

and wherein [symbol] is a bond to the antibody.

In some cases, the cyclooctyne group has the structure:

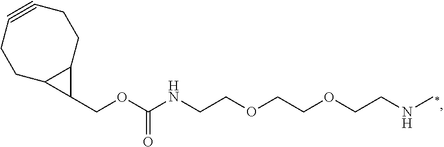 [SMILES: CNCCOCCOCCNC(=O)OCC1C2CCC#CCCC21]

and wherein [symbol] is a bond to the antibody.

In some embodiments, the attachment group has a formula comprising

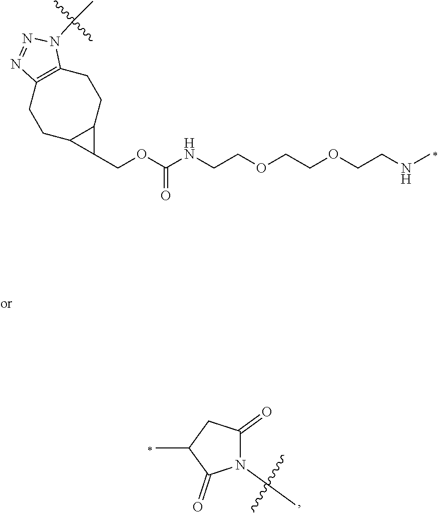

and wherein [symbol] is a bond to the antibody.

In some embodiments, the antibody is joined to the linker (L) by an attachment group selected from:

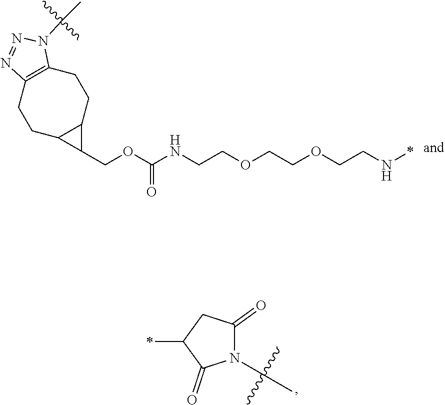

wherein [symbol] is a bond to the antibody, and wherein

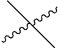

is a bond to the bridging spacer group. As used herein, the term “joined” refers to covalently attached to or covalently linked.

In some embodiments, the bridging spacer group is joined or covalently linked to a cleavable group.

In some embodiments, the bridging spacer group is —CO—CH₂—CH₂-PEG12-.

In some embodiments, the cleavable group is -pyrophosphate-CH₂—CH₂—NH₂—.

In some embodiments, the cleavable group is joined or covalently linked to the Bcl-xL inhibitor (D).

In some embodiments, the linker comprises: an attachment group, at least one bridging spacer group, a peptide group, and at least one cleavable group.

In some embodiments, the antibody-drug conjugate comprises a linker-drug moiety, -(L-D), is of the formula (B):

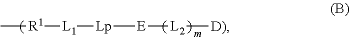

wherein R¹ is an attachment group, L₁ is a bridging spacer, Lp is a peptide group comprising 1 to 6 amino acid residues, E is a cleavable group, L₂ is a bridging spacer, m is 0 or 1; and D is a Bcl-xL inhibitor. In some cases, m is 1 and the bridging spacer comprises:

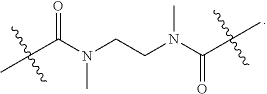 [SMILES: *C(=O)N(C)CCN(C)C(*)=O]

In some embodiments, the at least one bridging spacer comprises a PEG group. In some cases, the PEG group is selected from, PEG1, PEG2, PEG3, PEG4, PEG5, PEG6, PEG7, PEG8, PEG9, PEG10, PEG11, PEG12, PEG13, PEG14, and PEG15. In some cases, the at least one bridging spacer is selected from *—C(O)—CH₂—CH₂-PEG1-**, *—C(O)—CH₂-PEG3-**, *—C(O)—CH₂—CH₂-PEG12**, *—NH—CH₂—CH₂-PEG1-**, a polyhydroxyalkyl group, *—C(O)—N(CH₃)—CH₂—CH₂—N(CH₃)—C(O)—**, *—C(O)—CH₂—CH₂-PEG12-NH—C(O)CH₂—CH₂—**, and wherein ** indicates the point of direct or indirect attachment of the at least one bridging spacer to the attachment group and * indicates the point of direct or indirect attachment of the at least one bridging spacer to the peptide group.

In some embodiments, L₁ is selected from *—C(O)—CH₂—CH₂-PEG1-**, *—C(O)—CH₂-PEG3-**, *—C(O)—CH₂—CH₂-PEG12**, *—NH—CH₂—CH₂-PEG1-**, and a polyhydroxyalkyl group, wherein ** indicates the point of direct or indirect attachment of L₁ to R¹ and * indicates the point of direct or indirect attachment of L₁ to Lp.

In some embodiments, m is 1 and L₂ is —C(O)—N(CH₃)—CH₂—CH₂—N(CH₃)—C(O)—.

In some embodiments, the peptide group comprises 1 to 12 amino acid residues. In some embodiments, the peptide group (Lp) comprises 1 to 10 amino acid residues. In some embodiments, the peptide group (Lp) comprises 1 to 8 amino acid residues. In some embodiments, the peptide group (Lp) comprises 1 to 6 amino acid residues. In some embodiments, the peptide group comprises 1 to 4 amino acid residues. In some embodiments, the peptide group comprises 1 to 3 amino acid residues. In some embodiments the peptide group comprises 1 to 2 amino acid residues. In some cases, the amino acid residues are selected from glycine (Gly), L-valine (Val), L-citrulline (Cit), L-cysteic acid (sulfo-Ala), L-lysine (Lys), L-isoleucine (Ile), L-phenylalanine (Phe), L-methionine (Met), L-asparagine (Asn), L-proline (Pro), L-alanine (Ala), L-leucine (Leu), L-tryptophan (Trp), and L-tyrosine (Tyr). For example, the peptide group may comprise Val-Cit, Phe-Lys, Val-Ala, Val-Lys, Leu-Cit, sulfo-Ala-Val-Cit, sulfo-Ala-Val-Ala, Gly-Gly-Gly, and/or Gly-Gly-Phe-Gly (SEQ ID NO: 68). In some embodiments, the peptide group (Lp) comprises 1 amino acid residue linked to a

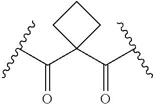 [SMILES: *C(=O)C1(C(*)=O)CCC1]

group. In some embodiments, the peptide group (Lp) comprises a group:

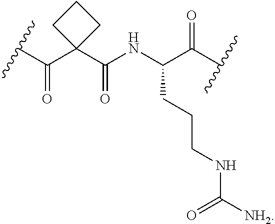 [SMILES: *C(=O)[C@H](CCCNC(N)=O)NC(=O)C1(C(*)=O)CCC1]

In some cases, the peptide group comprises a group selected from:

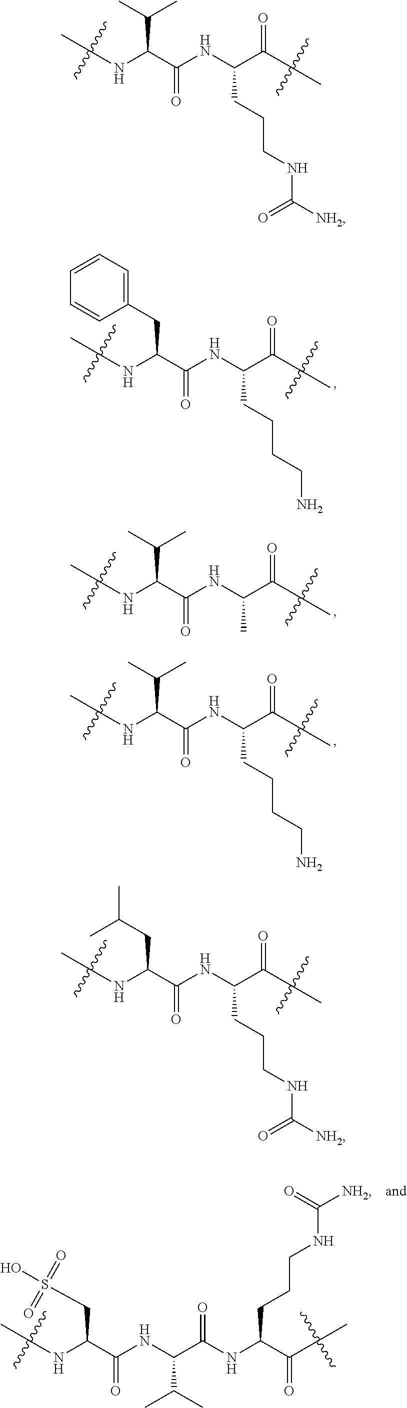

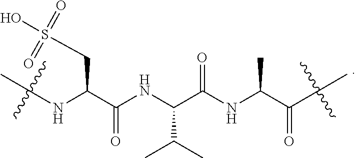 [SMILES: *N[C@@H](CS(=O)(=O)O)C(=O)N[C@H](C(=O)N[C@@H](C)C(*)=O)C(C)C]

In some embodiments, the self-immolative group comprises para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium.

In some embodiments, m is 1 and the bridging spacer comprises

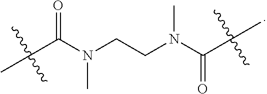 [SMILES: *C(=O)N(C)CCN(C)C(*)=O]

In some embodiments, the linker-drug moiety, -(L-D), is formed from a compound selected from:

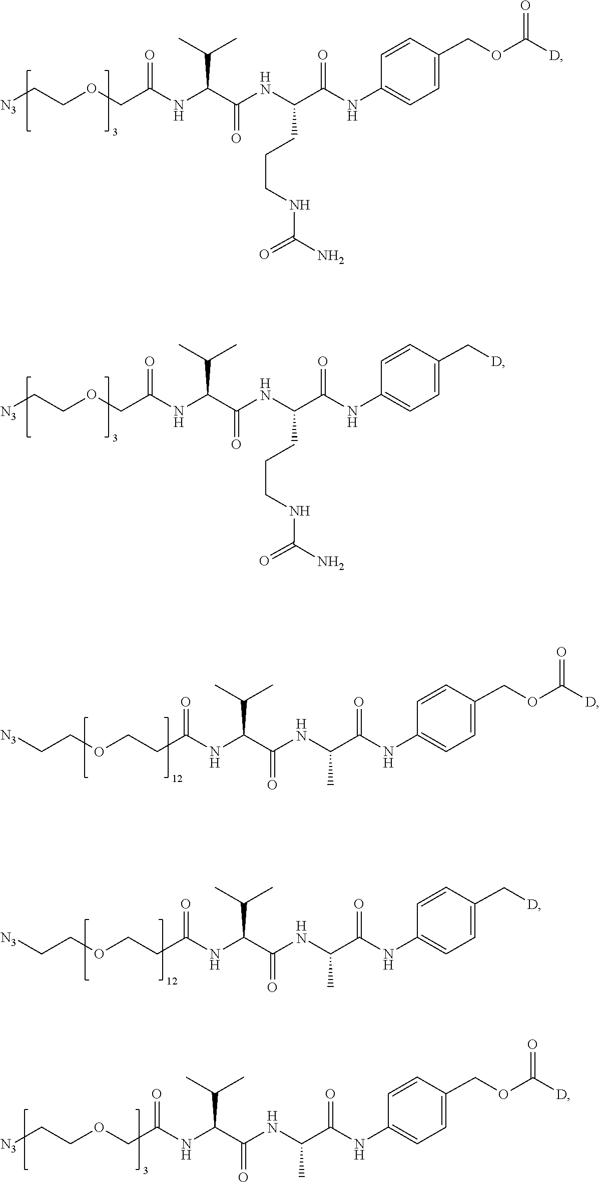

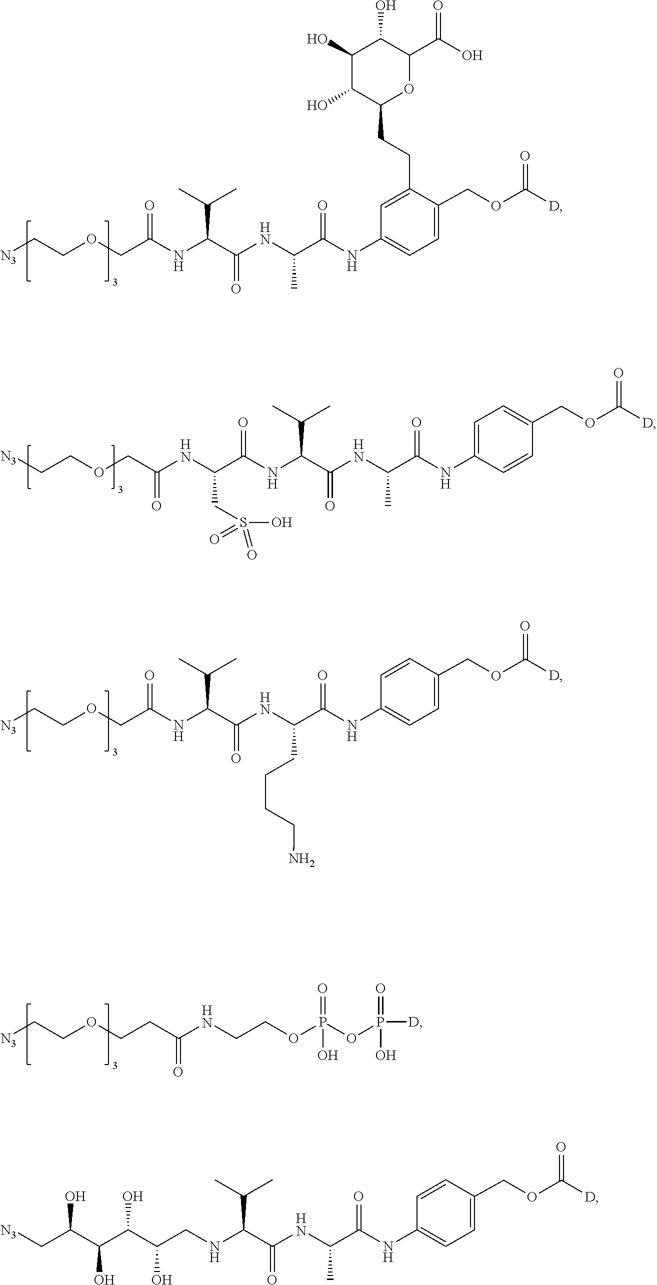

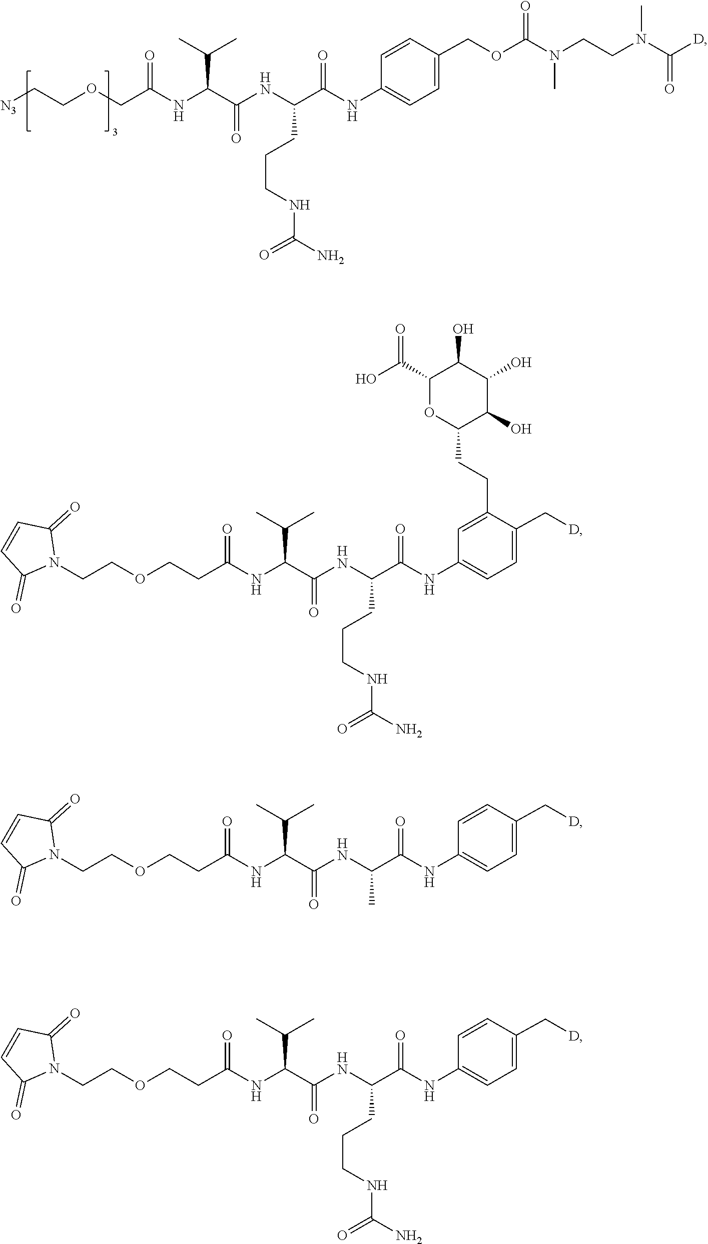

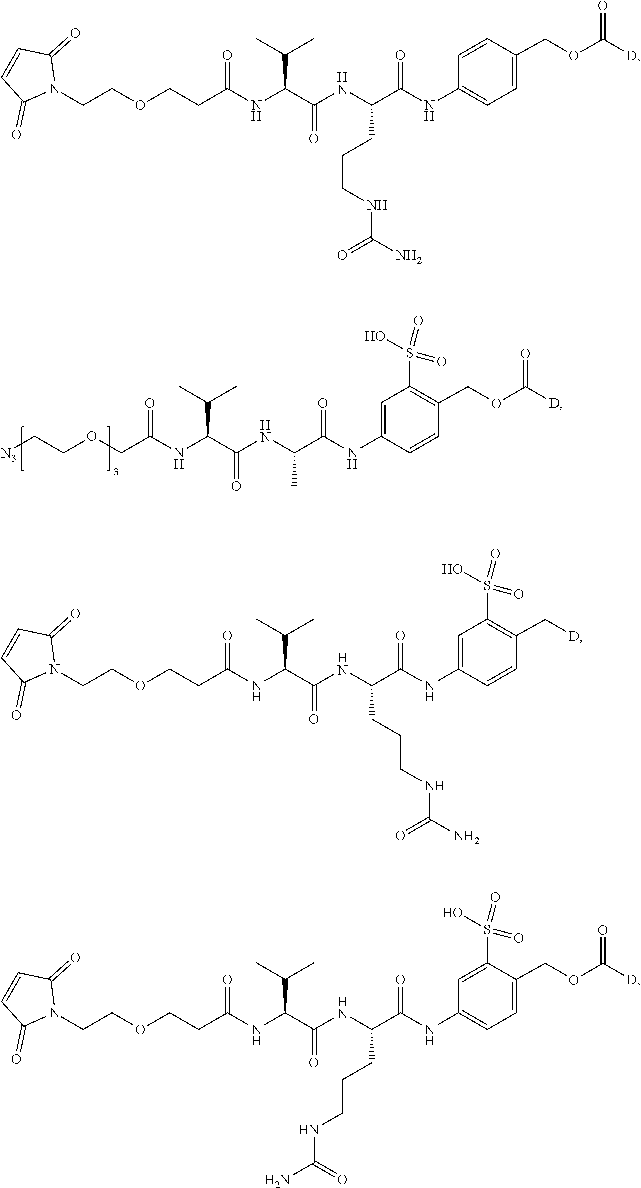

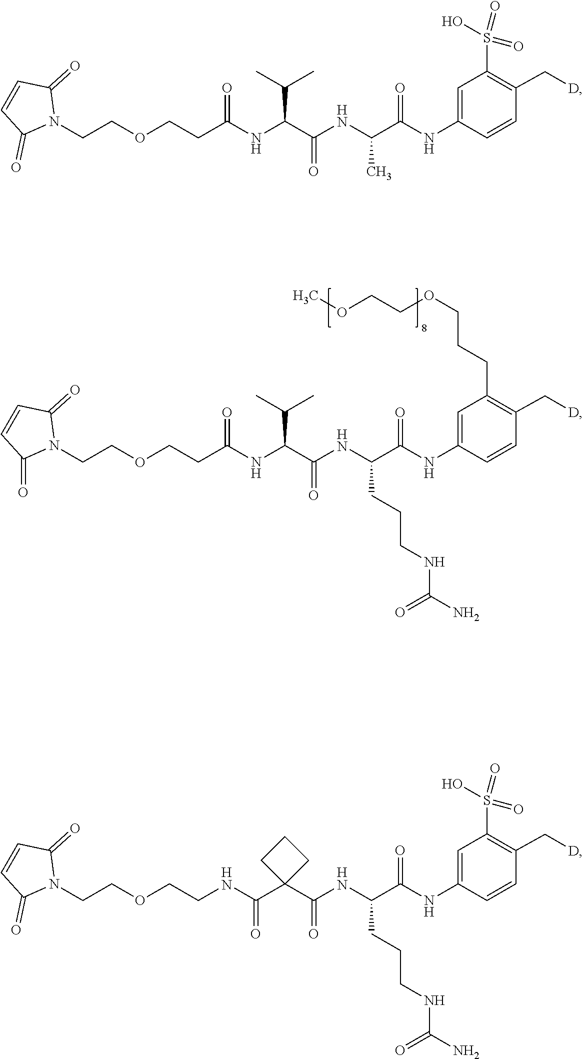

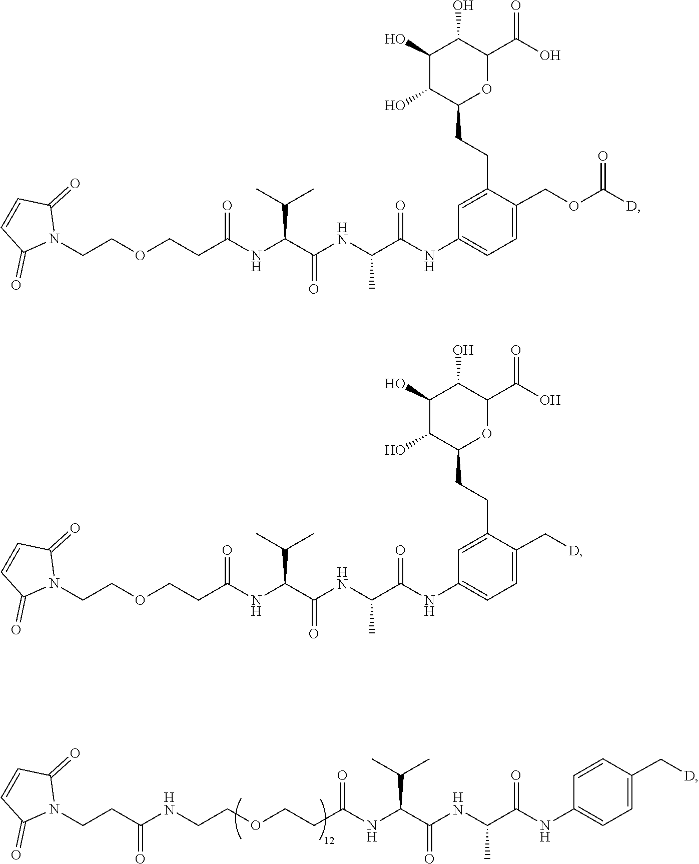

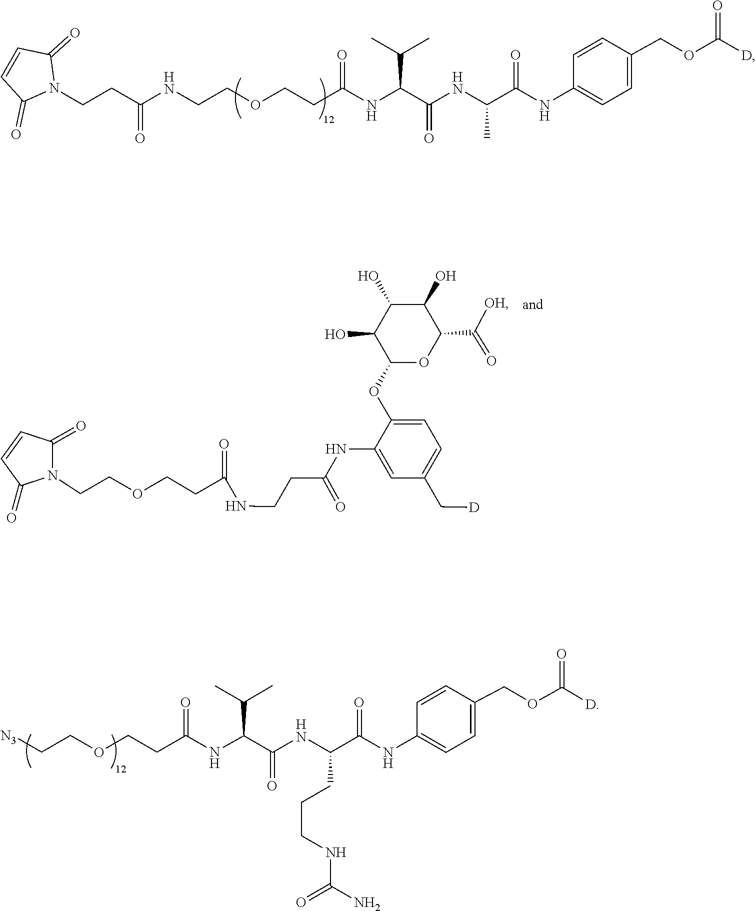

In some embodiments, the antibody-drug conjugate comprises the linker-drug group, -(L-D), which comprises a formula selected from:

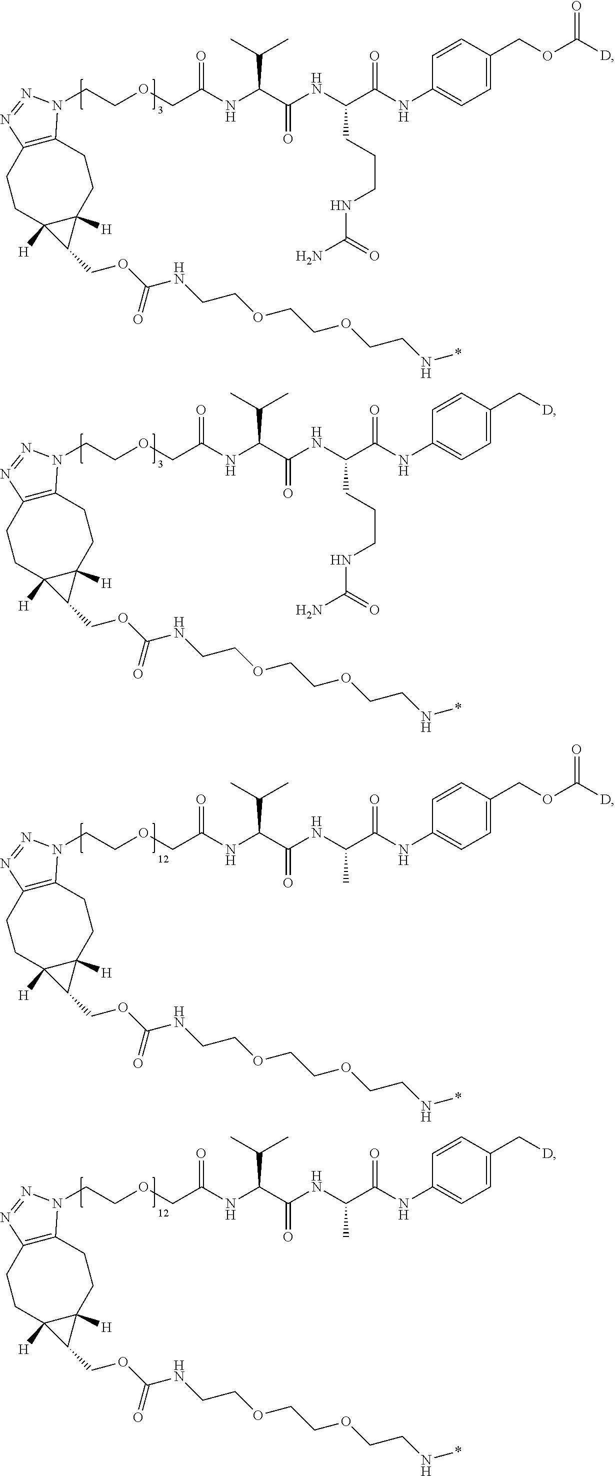

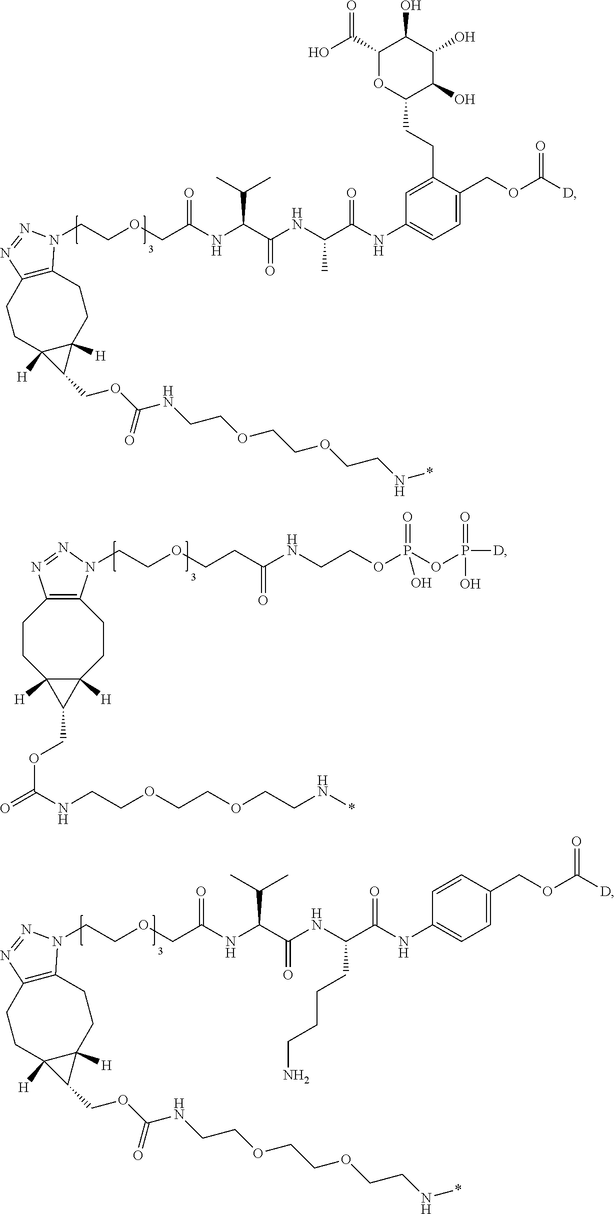

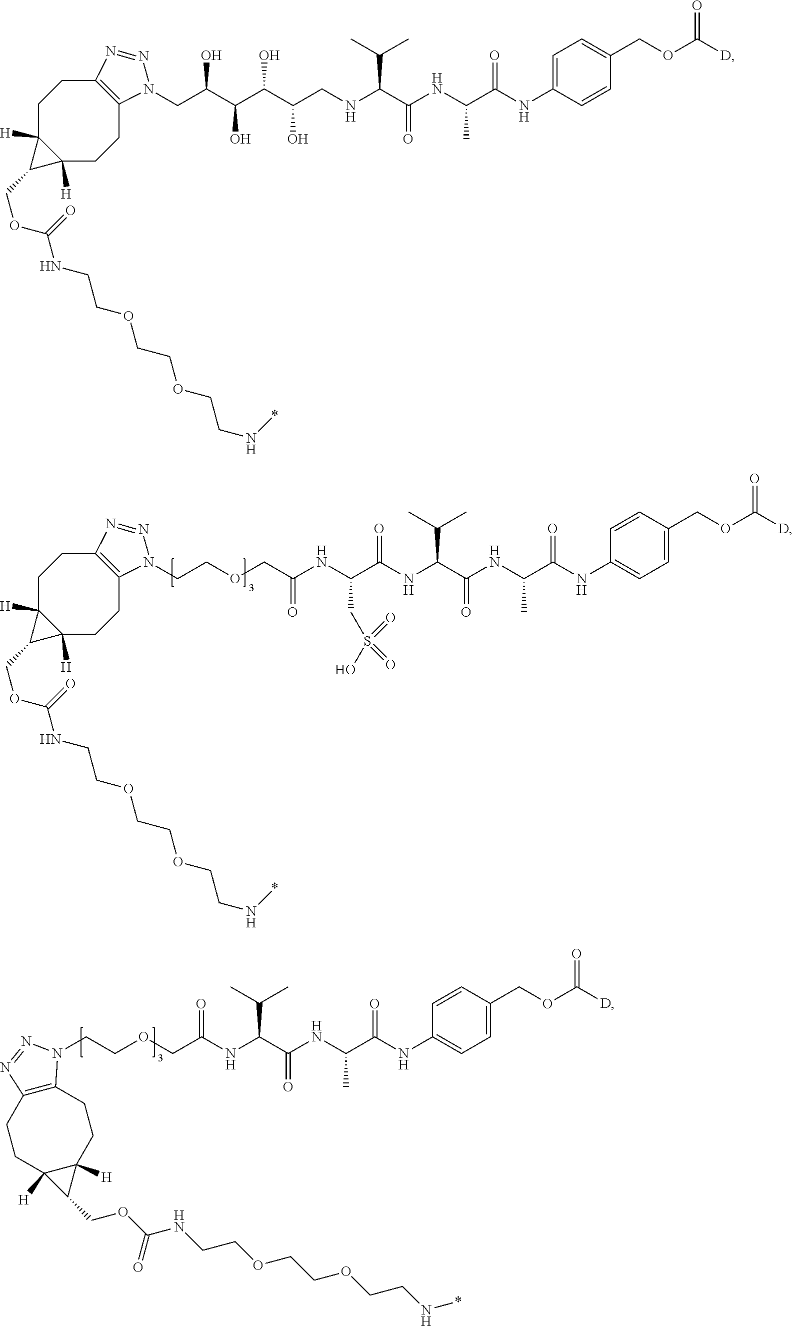

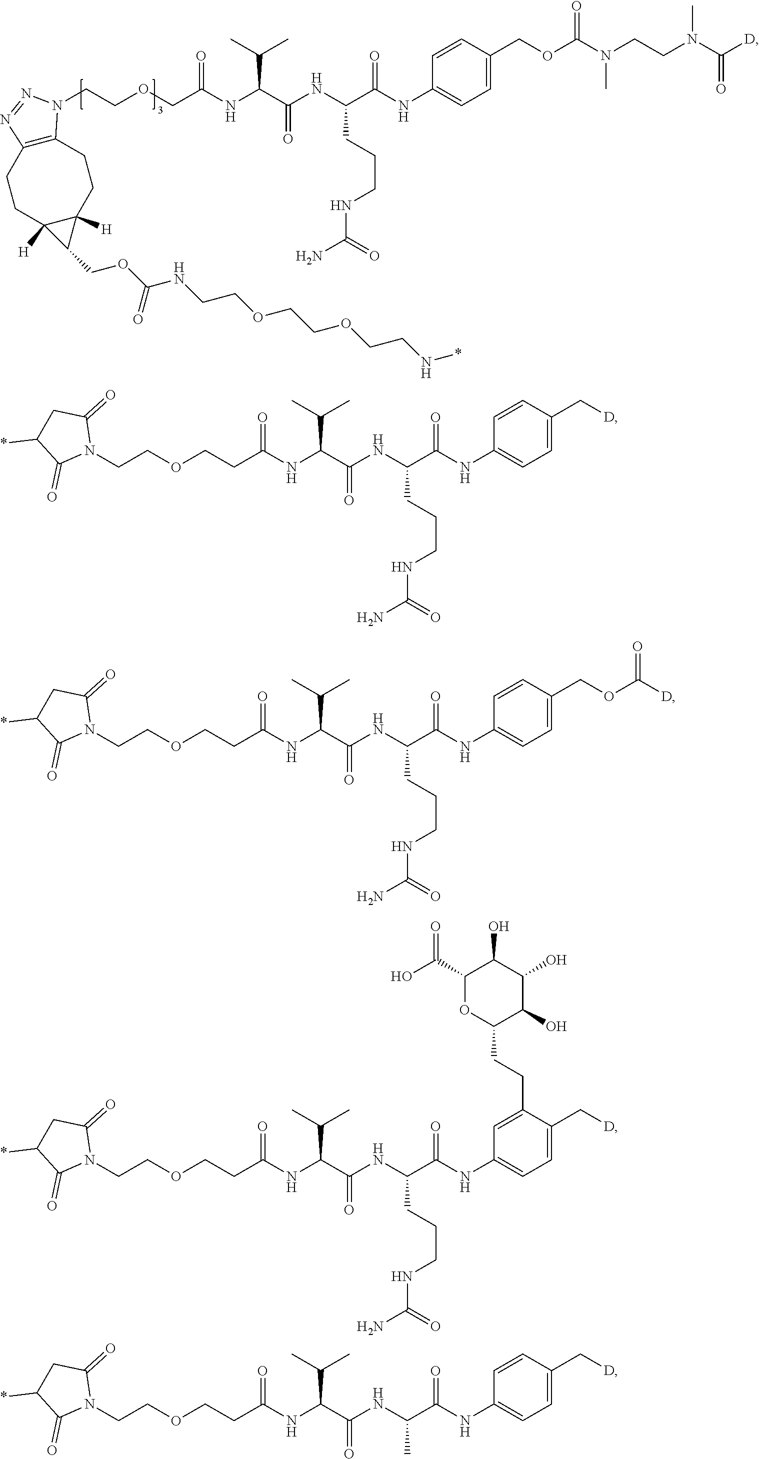

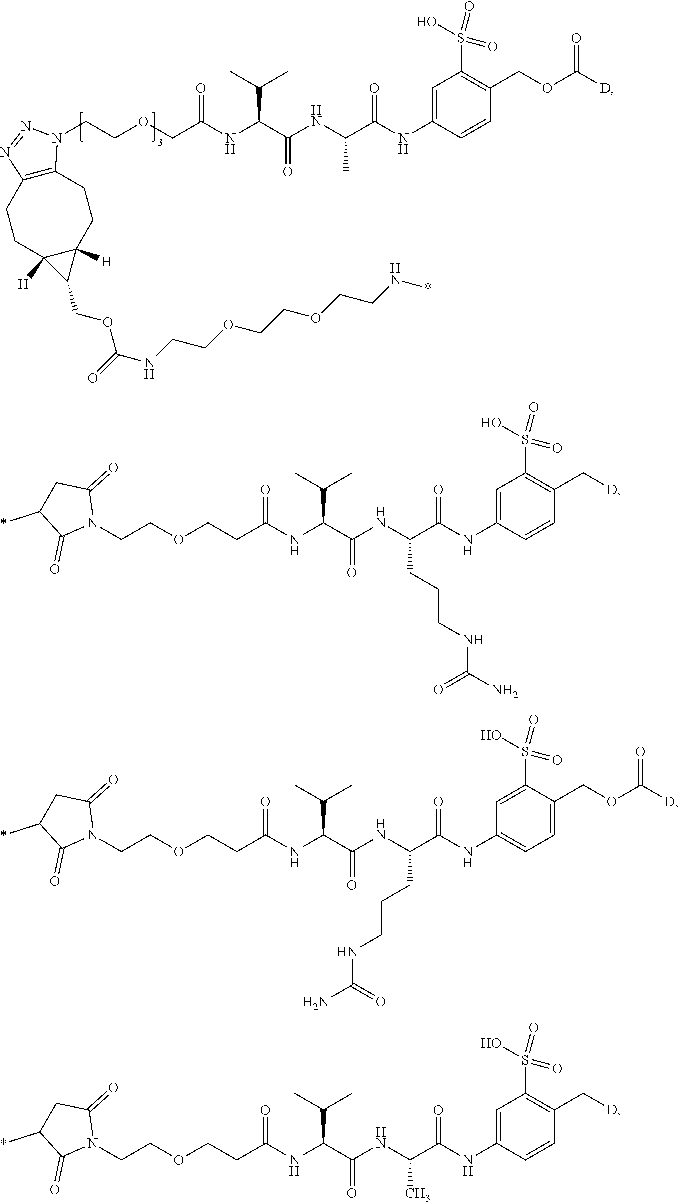

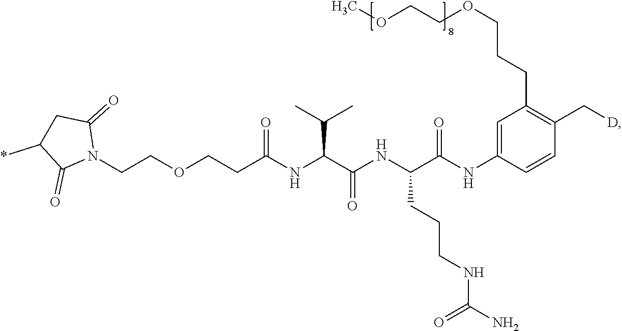

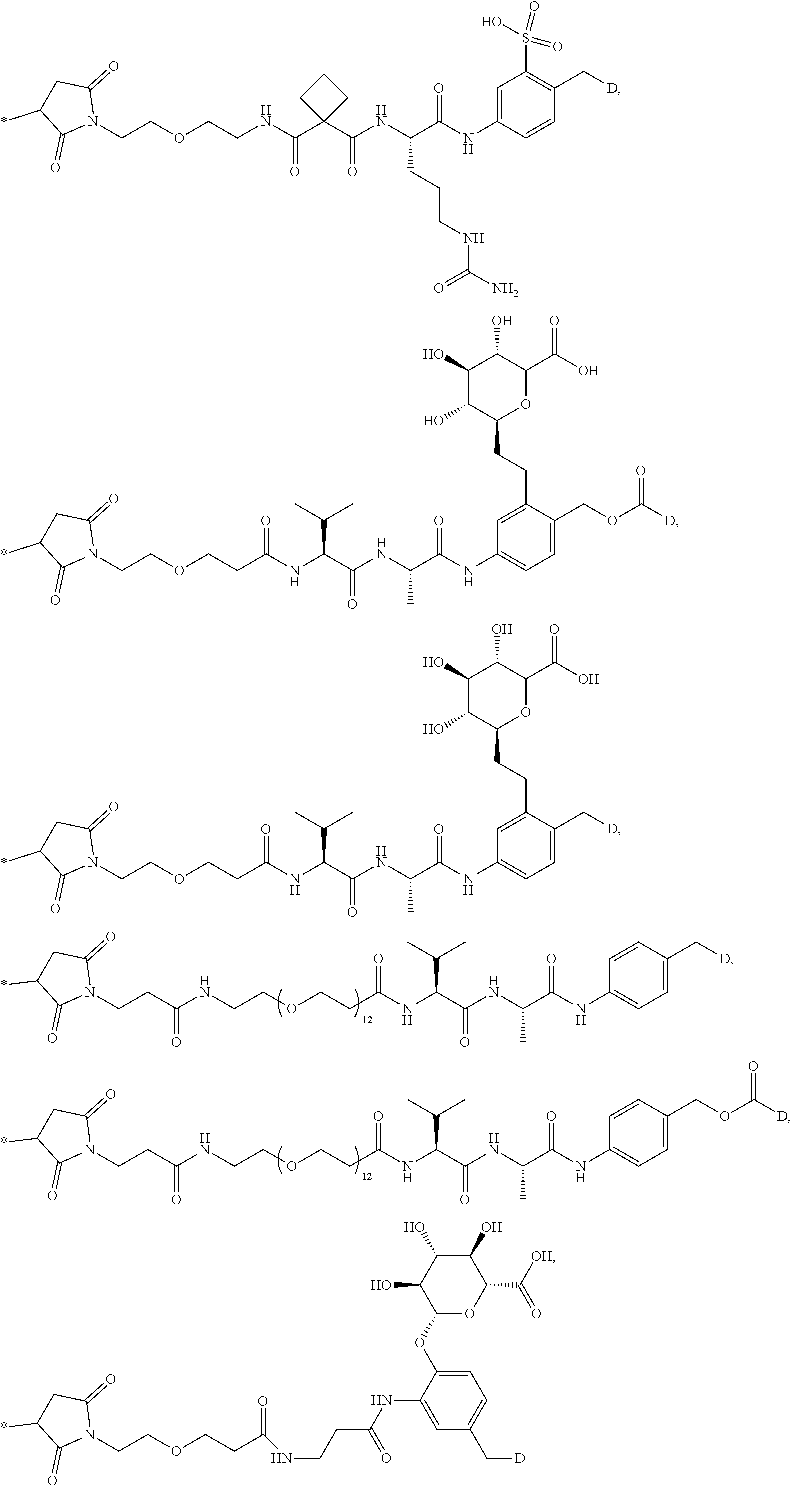

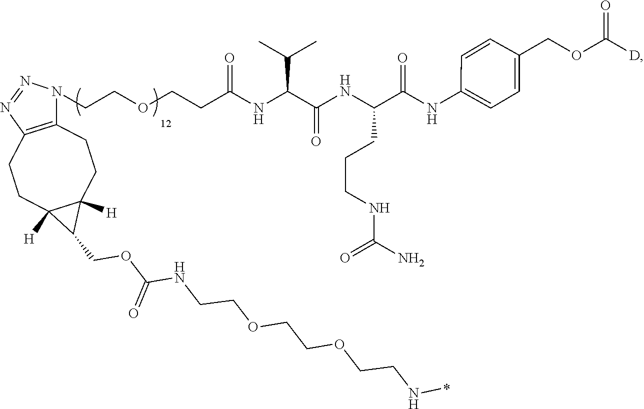

and
wherein [symbol] is a bond to the antibody.

In some embodiments, the antibody-drug conjugate comprises the linker drug group, -(L-D), which is of the formula (C):

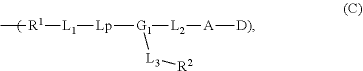

wherein: R¹ is an attachment group, L₁ is a bridging spacer; Lₚ is a peptide group comprising 1 to 6 amino acids; D is a Bcl-xL inhibitor; G₁-L₂-A is a self-immolative spacer; L₂ is a bond, a methylene, a neopentylene or a C₂-C₃ alkenylene; A is a bond, —OC(═O)—*,

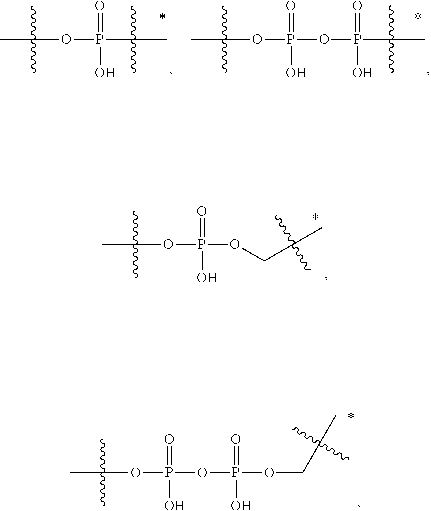 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; L₃ is a spacer moiety; and R² is a hydrophilic moiety.

In some embodiments, the antibody-drug conjugate comprises the linker drug group, -(L-D), which is of the formula (D):

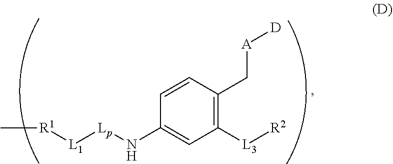

wherein: R¹ is an attachment group; L₁ is a bridging spacer; Lp is a peptide group comprising 1 to 6 amino acids; A is a bond, —OC(═O)—*,

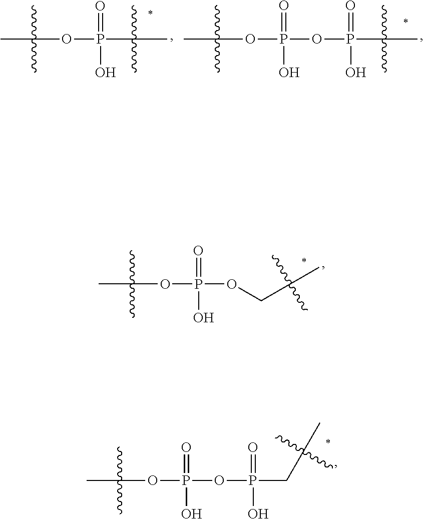 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; L₃ is a spacer moiety; and R² is a hydrophilic moiety.

In some embodiments, L₁ comprises:

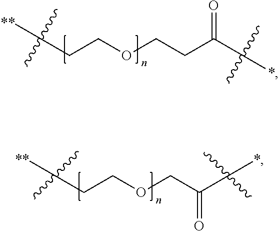

or *—CH(OH)CH(OH)CH(OH)CH(OH)—**, wherein each n is an integer from 1 to 12, wherein the * of L₁ indicates the point of direct or indirect attachment to Lp, and the ** of L₁ indicates the point of direct or indirect attachment to R¹.

In some embodiments, L₁ is

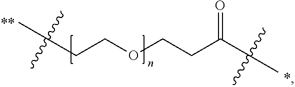

and n is an integer from 1 to 12

wherein the * of L₁ indicates the point of direct or indirect attachment to Lp, and the ** of L₁ indicates the point of direct or indirect attachment to R¹.

In some embodiments, L₁ is

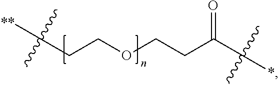

and n is 1, wherein the * of L₁ indicates the point of direct or indirect attachment to Lp, and the ** of L₁ indicates the point of direct or indirect attachment to R¹.

In some embodiments, L₁ is

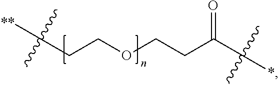

and n is 12, wherein the * of L₁ indicates the point of direct or indirect attachment to Lp, and the ** of L₁ indicates the point of direct or indirect attachment to R¹.

In some embodiments, L₁ is

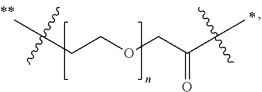

and n is an integer from 1 to 12, wherein the * of L₁ indicates the point of direct or indirect attachment to Lp, and the ** of L₁ indicates the point of direct or indirect attachment to R¹.

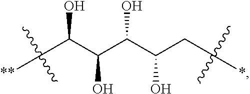

In some embodiments, L₁ comprises wherein the * of L₁ indicates the point of direct or indirect attachment to Lp, and the ** of L₁ indicates the point of direct or indirect attachment to R¹.

In some embodiments, L₁ is a bridging spacer comprising:
*—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of direct or indirect attachment to Lp, and the ** of L₁ indicates the point of direct or indirect attachment to R¹,

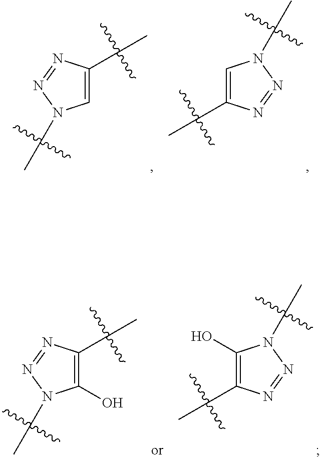 [SMILES: *c1cn(*)nn1 ; *c1cn(*)nn1 ; *c1nnn(*)c1O ; *c1nnn(*)c1O]

wherein X₁ is
and
each m is independently selected from 1, 2, 3, 4, 5, 6, 7, 8, 9 and 10;
each n is independently selected from 1, 2, 3, 4, 5, 6, 7, 8, 9 and 10; and
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30.

In some embodiments, R² is a hydrophilic moiety comprising polyethylene glycol, polyalkylene glycol, a polyol, a polysarcosine, a sugar, an oligosaccharide, a polypeptide, C₂-C₆ alkyl substituted with 1 to 3

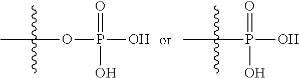 [SMILES: *OP(=O)(O)O ; *P(=O)(O)O]

groups, or C₂-C₆alkyl substituted with 1 to 2 substituents independently selected from —OC(═O)NHS(O)₂NHCH₂CH₂OCH₃, —NHC(═O)C₁₋₄alkylene-P(O)(OCH₂CH₃)₂ and —COOH groups. In some embodiments, R² is

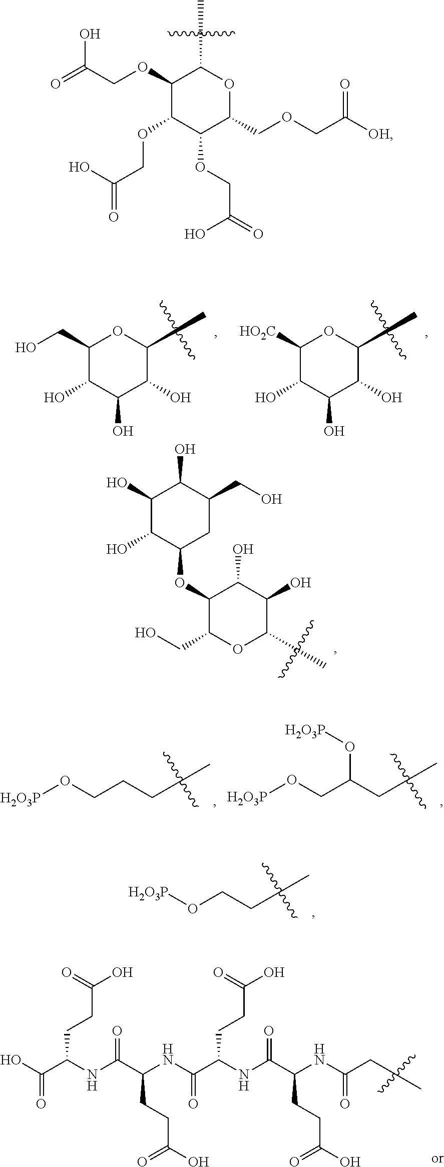

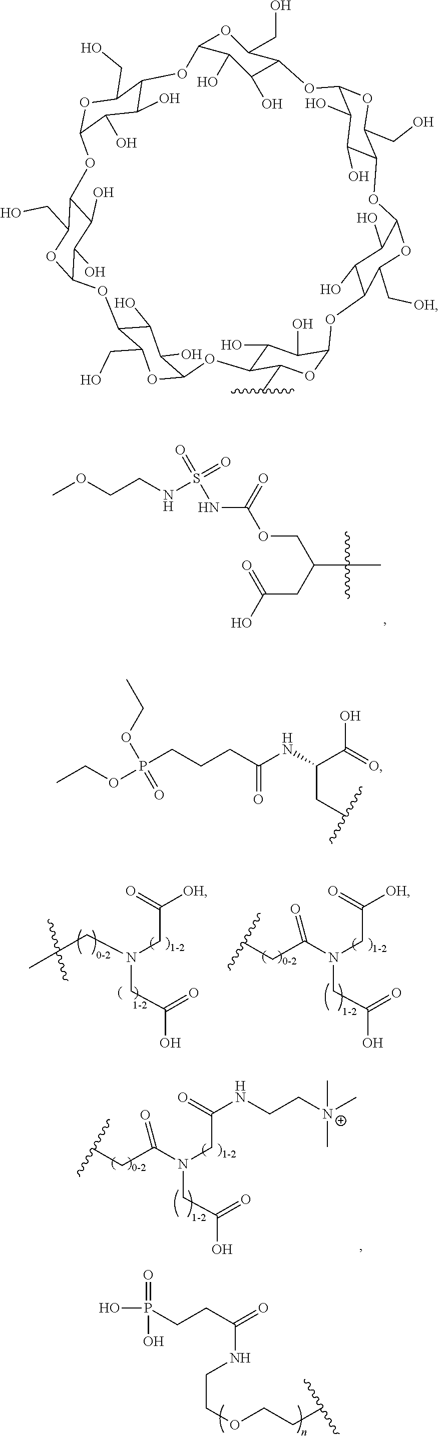

wherein n is an integer between 1 and 6,

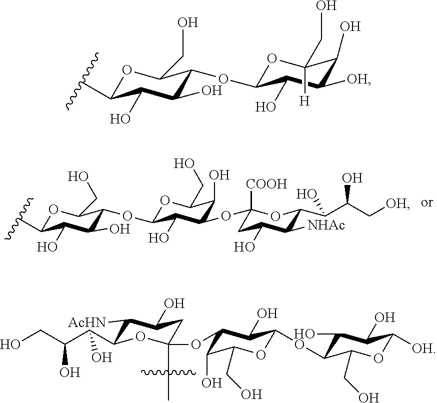

In some embodiments, the hydrophilic moiety comprises a polyethylene glycol of formula:

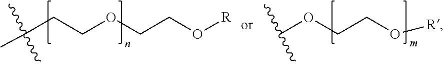

wherein R is H, —CH₃ CH₂CH₂NHC(═O)ORₐ, —CH₂CH₂NHC(═O)Rₐ, or —CH₂CH₂C(═O)ORₐ, R′ is OH, —OCH₃, CH₂CH₂NHC(═O)ORₐ, —CH₂CH₂NHC(═O)Rₐ, or —OCH₂CH₂C(═O)ORₐ, and each of m and n is an integer between 2 and 25 (e.g., between 3 and 25).

In some embodiments,

the hydrophilic moiety comprises

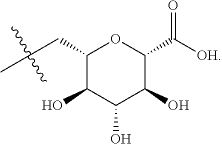 [SMILES: *C[C@@H]1O[C@H](C(=O)O)[C@@H](O)[C@H](O)[C@H]1O]

In some embodiments, the hydrophilic moiety comprises a polysarcosin, e.g., with the following moiety

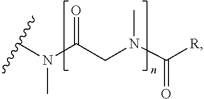

wherein n is an integer between 3 and 25; and R is H, —CH₃ or —CH₂CH₂C(═O)OH.

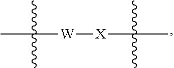

In some embodiments, L₃ is a spacer moiety having the structure wherein:
W is —CH₂—, —CH₂O—, —CH₂N(R^{b})C(═O)O—, —NHC(═O)C(R^{b})₂NHC(═O)O—, —NHC(═O)C(R^{b})₂NH—, —NHC(═O)C(R^{b})₂NHC(═O)—, —CH₂N(X—R²)C(═O)O—, —C(═O)N(X—R²)—, —CH₂N(X—R²)C(═O)—, —C(═O)NR^{b}—, —C(═O)NH—, —CH₂NR^{b}C(═O)—, —CH₂NR^{b}C(═O)NH—, —CH₂NR^{b}C(═O)NR^{b}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{b} is independently selected from H, C₁-C₆alkyl, and C₃-C₈ cycloalkyl; and
X is a bond, triazolyl or —CH₂-triazolyl-, wherein X is connected to R².

In some embodiments, L₃ is a spacer moiety having the structure

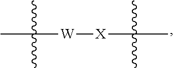

wherein:
W is —CH₂—, —CH₂O—, —CH₂N(R^{b})C(═O)O—, —NHC(═O)C(R^{b})₂NHC(═O)O—, —NHC(═O)C(R^{b})₂NH—, —NHC(═O)C(R^{b})₂NHC(═O)—, —CH₂N(X—R²)C(═O)O—, —C(═O)N(X—R²)—, —CH₂N(X—R²)C(═O)—, —C(═O)NR^{b}—, —C(═O)NH—, —CH₂NR^{b}C(═O)—, —CH₂NR^{b}C(═O)NH—, —CH₂NR^{b}C(═O)NR^{b}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{b} is independently selected from H, C₁-C₆alkyl, and C₃-C₈ cycloalkyl; and
X is —CH₂-triazolyl-C₁₋₄ alkylene-OC(O)NHS(O)₂NH—, —C₄₋₆ cycloalkylene-OC(O)NHS(O)₂NH—, —(CH₂CH₂O)ₙ—C(O)NHS(O)₂NH—, (CH₂CH₂O)ₙ—C(O)NHS(O)₂NH—(CH₂CH₂O)ₙ—, —CH₂-triazolyl-C₁₋₄ alkylene-OC(O)NHS(O)₂NH—(CH₂CH₂O)ₙ—, or —C₄₋₆ cycloalkylene-OC(O)NHS(O)₂NH—(CH₂CH₂O)ₙ—, wherein each n independently is 1, 2, or 3 and wherein X is connected to R².

In some embodiments, the attachment group is formed by a reaction comprising at least one reactive group. In some cases, the attachment group is formed by reacting: a first reactive group that is attached to the linker, and a second reactive group that is attached to the antibody or is an amino acid residue of the antibody.

In some embodiments, at least one of the reactive groups comprises:
a thiol,
a maleimide,
a haloacetamide,
an azide,
an alkyne,
a cyclcooctene,
a triaryl phosphine,
an oxanobornadiene,
a cyclooctyne,
a diaryl tetrazine,
a monoaryl tetrazine,
a norbornene,
an aldehyde,
a hydroxylamine,
a hydrazine,
NH₂—NH—C(═O)—,
a ketone,
a vinyl sulfone,
an aziridine,
an amino acid residue,

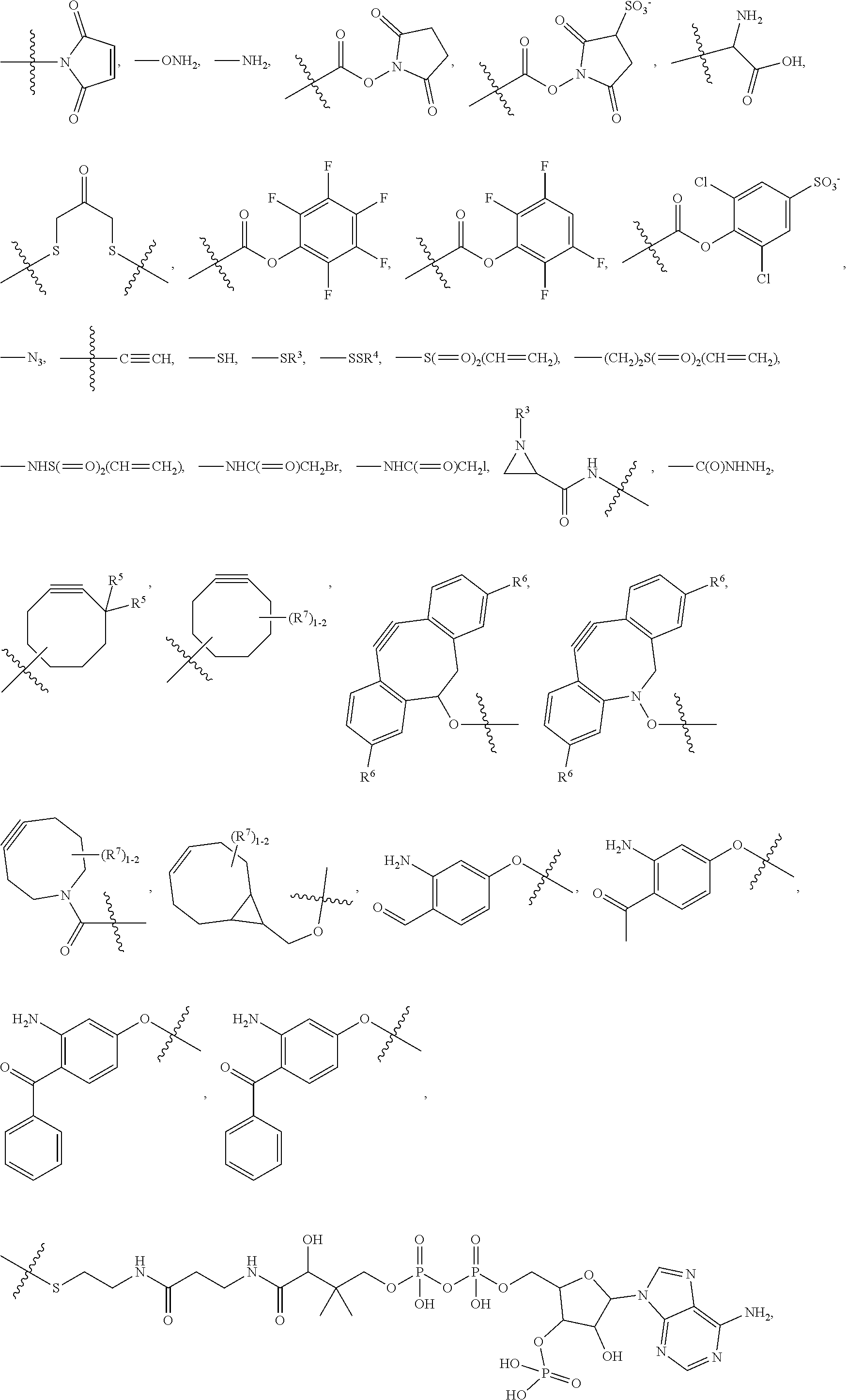

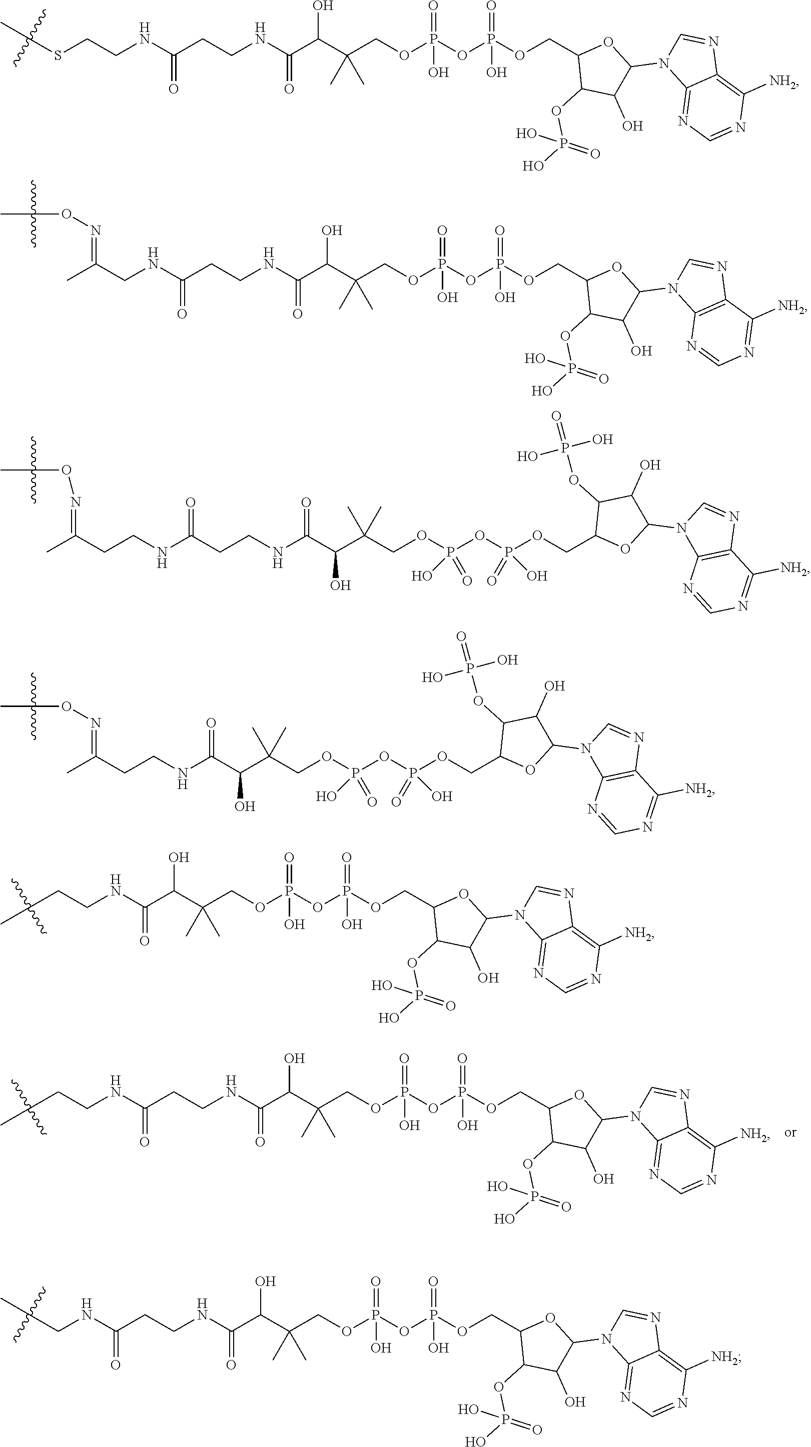

wherein:
each R³ is independently selected from H and C₁-C₆alkyl;
each R⁴ is 2-pyridyl or 4-pyridyl;
each R⁵ is independently selected from H, C₁-C₆alkyl, F, Cl, and —OH;
each R⁶ is independently selected from H, C₁-C₆alkyl, F, Cl, —NH₂, —OCH₃, —OCH₂CH₃, —N(CH₃)₂, —CN, —NO₂ and —OH;
each R⁷ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH.

In some embodiments, the first reactive group and second reactive group comprise:
a thiol and a maleimide,
a thiol and a haloacetamide,
a thiol and a vinyl sulfone,
a thiol and an aziridine,
an azide and an alkyne,
an azide and a cyclooctyne,
an azide and a cyclooctene,
an azide and a triaryl phosphine,
an azide and an oxanobornadiene,
a diaryl tetrazine and a cyclooctene,
a monoaryl tetrazine and a nonbornene,
an aldehyde and a hydroxylamine,
an aldehyde and a hydrazine,
an aldehyde and NH₂—NH—C(═O)—,
a ketone and a hydroxylamine,
a ketone and a hydrazine,
a ketone and NH₂—NH—C(═O)—,
a hydroxylamine and

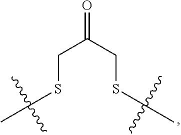 [SMILES: *SCC(=O)CS*]

an amine and

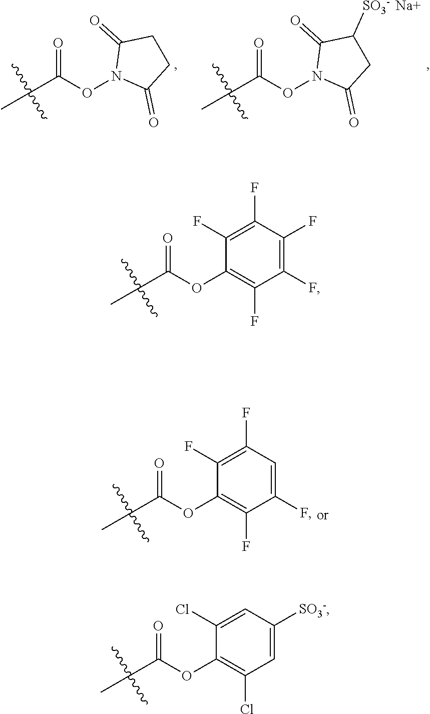

or
a CoA or CoA analogue and a serine residue.

In some embodiments, the attachment group comprises a group selected from:

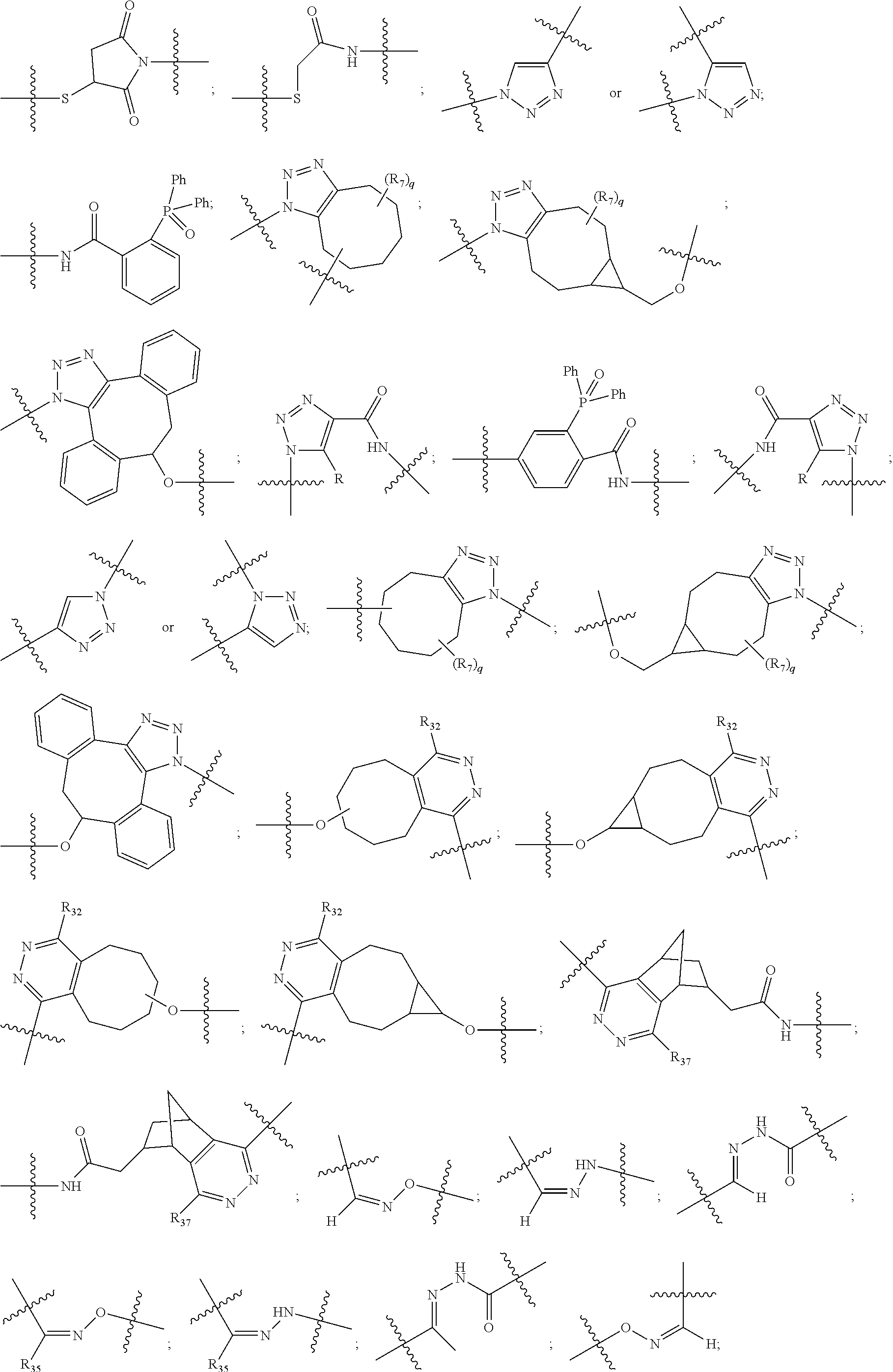

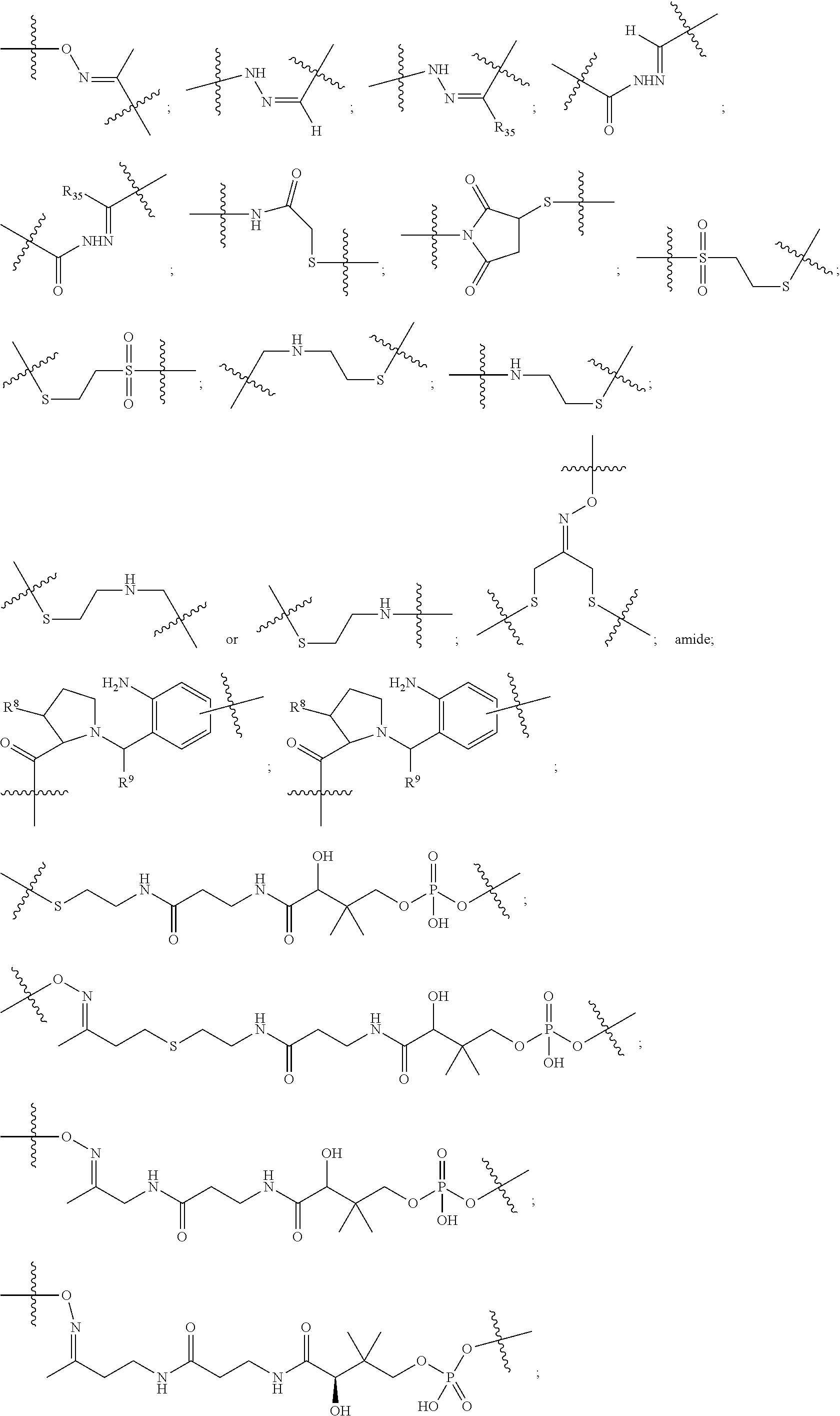

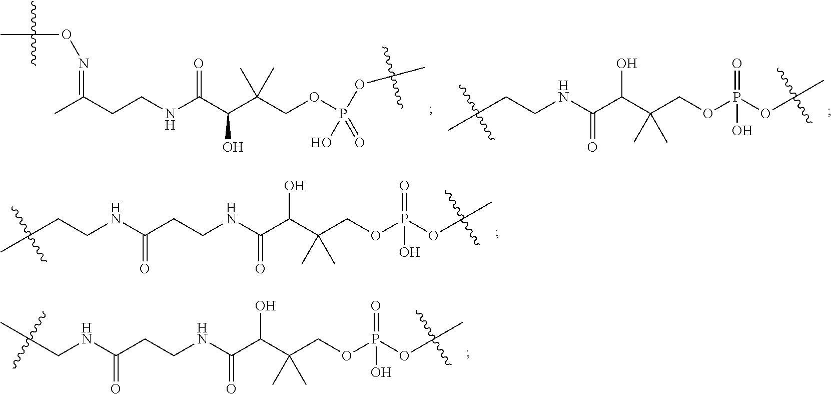

and
disulfide,
   wherein:
R³² is H, C₁₋₄ alkyl, phenyl, pyrimidine or pyridine;
R³⁵ is H, C₁₋₆ alkyl, phenyl or C₁₋₄ alkyl substituted with 1 to 3 —OH groups; each R⁷ is independently selected from H, C₁₋₆ alkyl, fluoro, benzyloxy substituted with —C(═O)OH, benzyl substituted with —C(═O)OH, C₁₋₄ alkoxy substituted with —C(═O)OH and C₁₋₄ alkyl substituted with —C(═O)OH;
R³⁷ is independently selected from H, phenyl and pyridine;
q is 0, 1, 2 or 3;
R⁸ is H or methyl; and
R⁹ is H, —CH₃ or phenyl.

In some embodiments, the peptide group (Lp) comprises 1 to 6 amino acid residues. In some embodiments, the peptide group (Lp) comprises 1 to 4 amino acid residues. In some embodiments, the peptide group comprises 1 to 3 amino acid residues. In some embodiments, the peptide group comprises 1 to 2 amino acid residues. In some embodiments, the amino acid residues are selected from glycine (Gly), L-valine (Val), L-citrulline (Cit), L-cysteic acid (sulfo-Ala), L-lysine (Lys), L-isoleucine (Ile), L-phenylalanine (Phe), L-methionine (Met), L-asparagine (Asn), L-proline (Pro), L-alanine (Ala), L-leucine (Leu), L-tryptophan (Trp), and L-tyrosine (Tyr). In some embodiments, the peptide group comprises Val-Cit, Phe-Lys, Val-Ala, Val-Lys, Leu-Cit, sulfo-Ala-Val-Cit, sulfo-Ala-Val-Ala, Gly-Gly-Gly, and/or Gly-Gly-Phe-Gly (SEQ ID NO: 78).

In some embodiments, Lp is selected from:

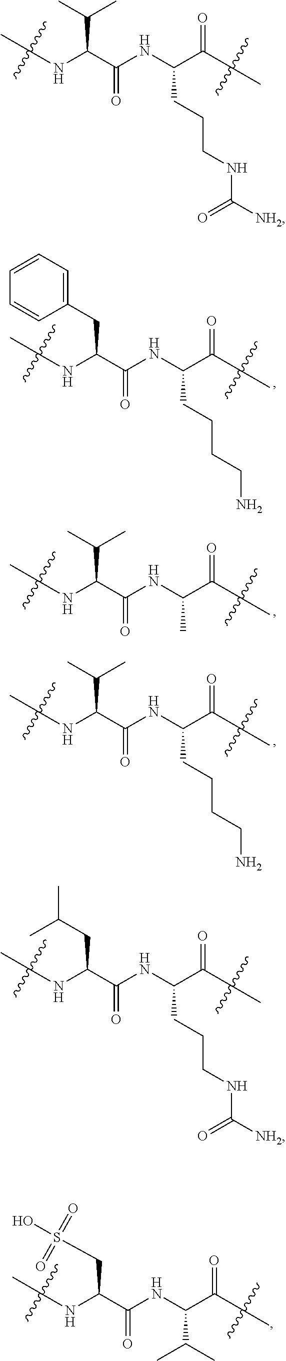

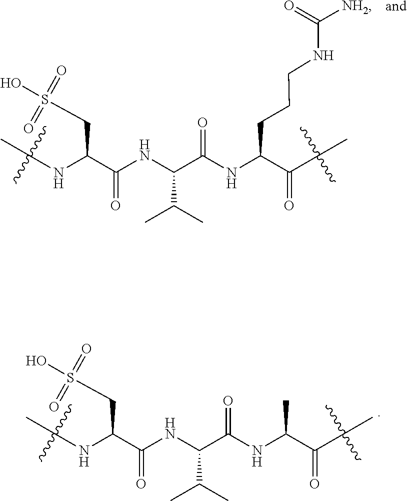 [SMILES: *N[C@@H](CS(=O)(=O)O)C(=O)N[C@H](C(=O)N[C@@H](CCCNC(N)=O)C(*)=O)C(C)C ; "and" ; *N[C@@H](CS(=O)(=O)O)C(=O)N[C@H](C(=O)N[C@@H](C)C(*)=O)C(C)C]

In some embodiments, the linker-drug group -(L-D) comprises or is formed from a compound of formula:

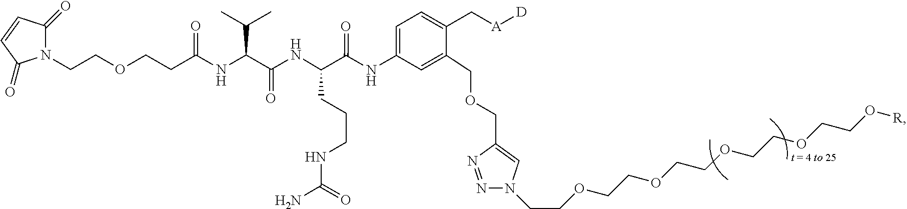

wherein:
R is H, —CH₃ or —CH₂CH₂C(═O)OH;
A is a bond, —OC(═O)—*,

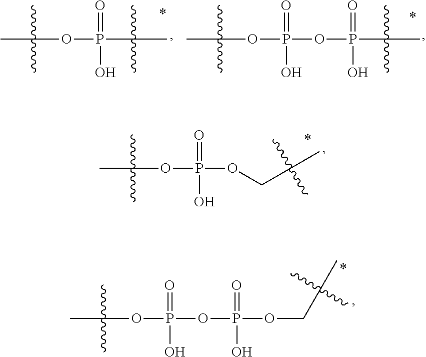 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

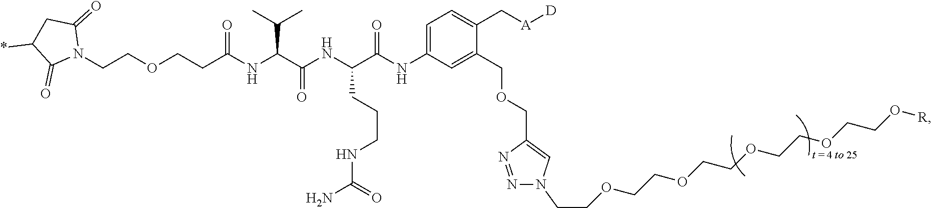

wherein: [symbol] is a bond to the antibody; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

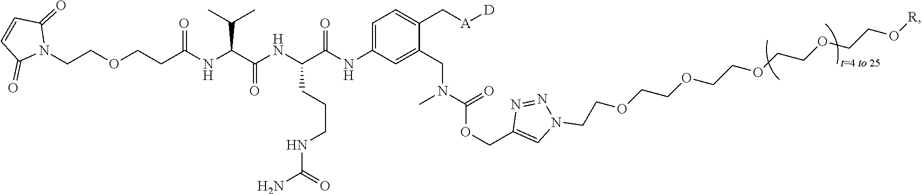

wherein:
R is H, —CH₃ or —CH₂CH₂C(═O)OH;
A is a bond, —OC(—O)—*,

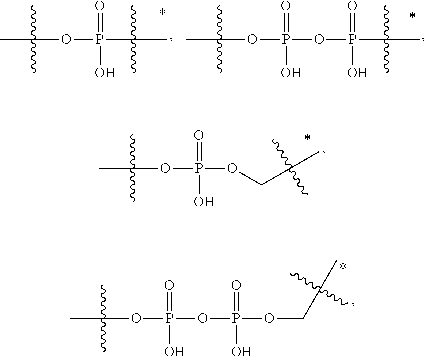 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(—O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

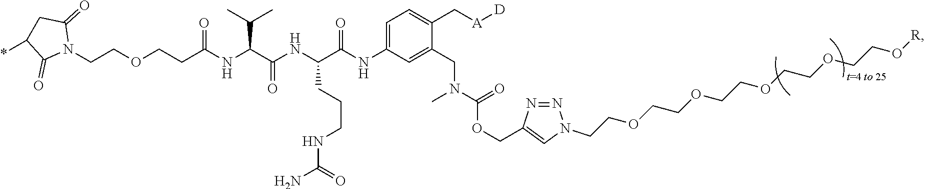

wherein: [symbol] is a bond to the antibody; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

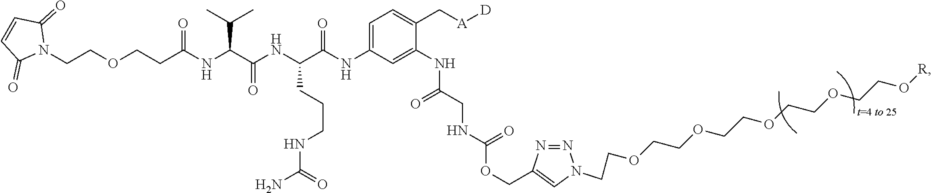

wherein:
R is H, —CH₃ or —CH₂CH₂C(═O)OH;
A is a bond, —OC(═O)—*,

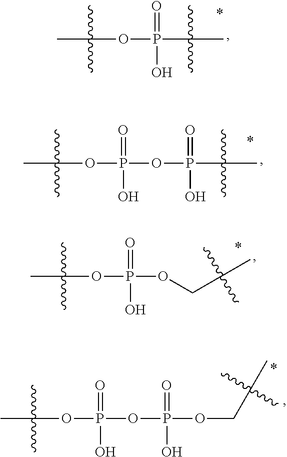 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

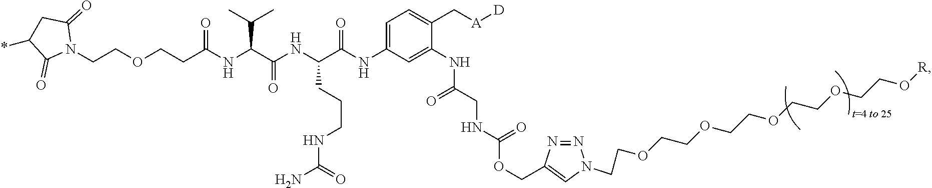

wherein: [symbol] is a bond to the antibody; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

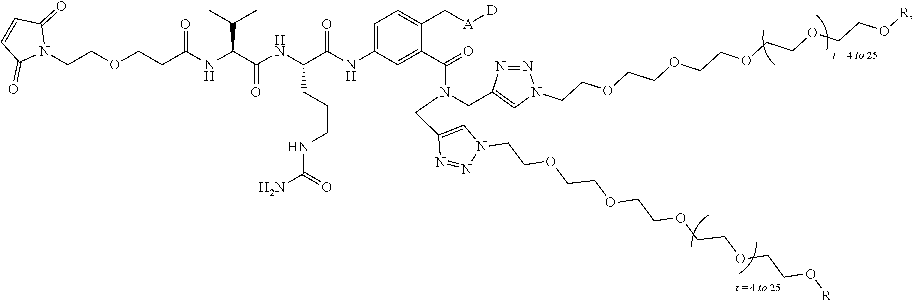

wherein:
each R is independently selected from H, —CH₃, and —CH₂CH₂C(═O)OH;
A is a bond, —OC(═O)—*,

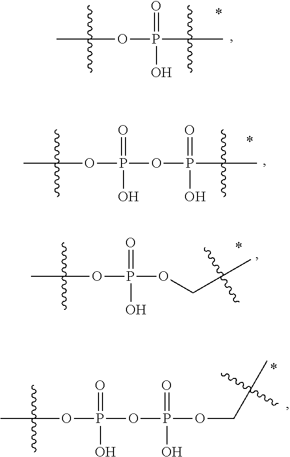 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(—O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

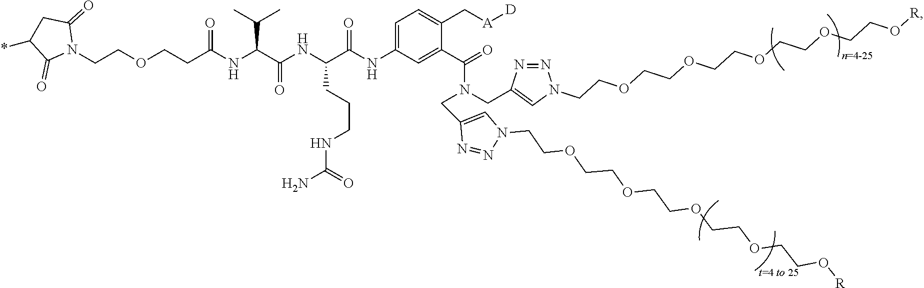

wherein: [symbol] is a bond to the antibody; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

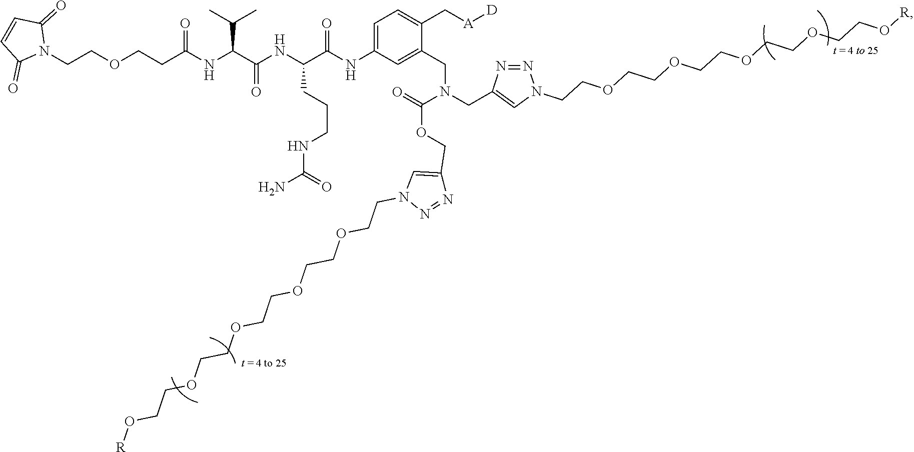

wherein:
each R is independently selected from H, —CH₃, and —CH₂CH₂C(═O)OH;
A is a bond, —OC(═O)—*,

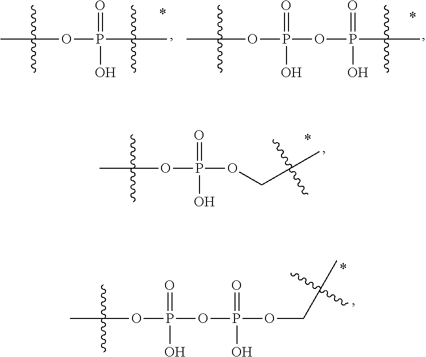 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

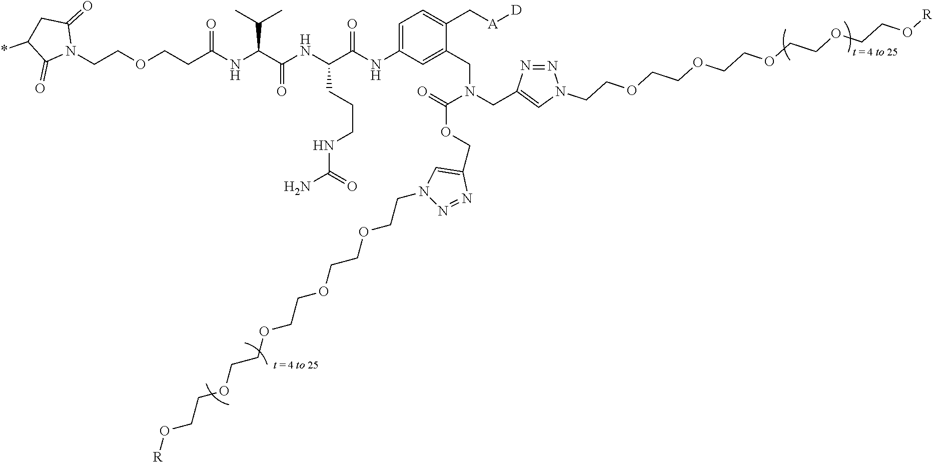

wherein: [symbol] is a bond to the antibody; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

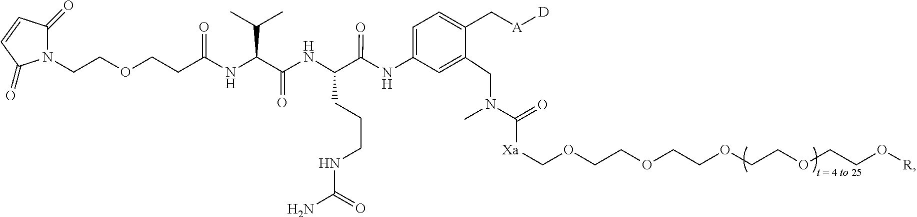

wherein:
Xa is —CH₂—, —OCH₂—, —NHCH₂— or —NRCH₂— and each R independently is H, —CH₃ or —CH₂CH₂C(═O)OH;
A is a bond, —OC(═O)—*,

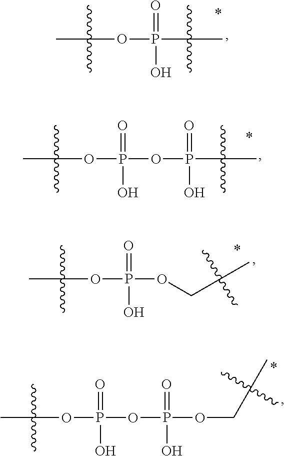 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or
—OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

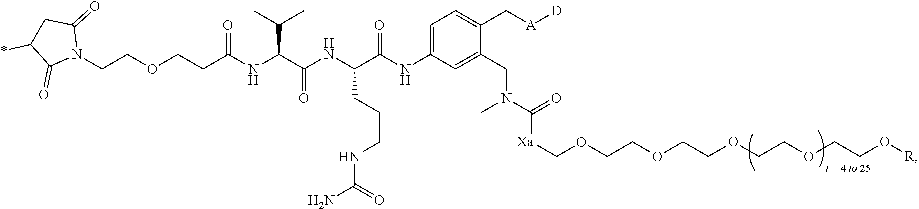

wherein: [symbol] is a bond to the antibody; and Xa, A, D and R are as defined above. In some embodiments, Xa is —CH₂— or —NHCH₂—; A is a bond or —OC(═O)—*; and R is —CH₃ or —CH₂CH₂C(═O)OH.

In some embodiments, the linker-drug group -(L-D) comprises or is formed from a compound of formula:

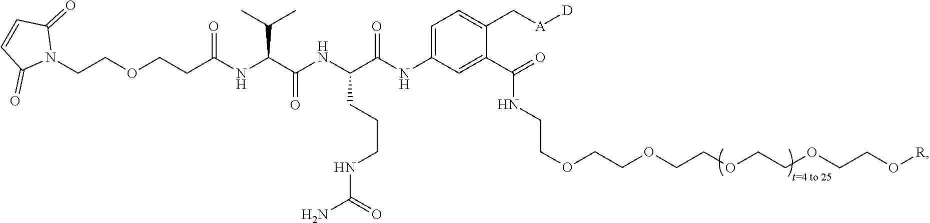

wherein:
R is H, —CH₃ or —CH₂CH₂C(═O)OH;
A is a bond, —OC(═O)—*,

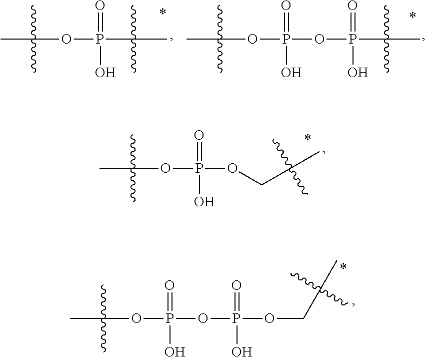 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or
—OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

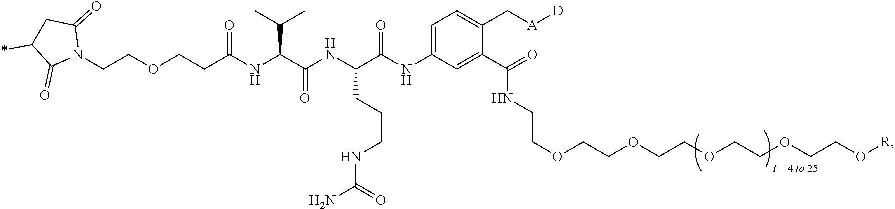

wherein: [symbol] is a bond to the antibody; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

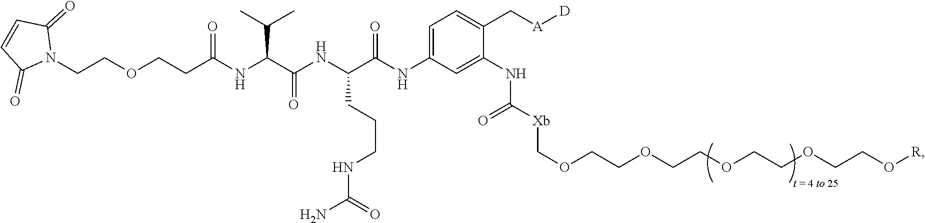

wherein:
Xb is —CH₂—, —OCH₂—, —NHCH₂— or —NRCH₂— and each R independently is H, —CH₃ or —CH₂CH₂C(═O)OH;
A is a bond, —OC(═O)—*,

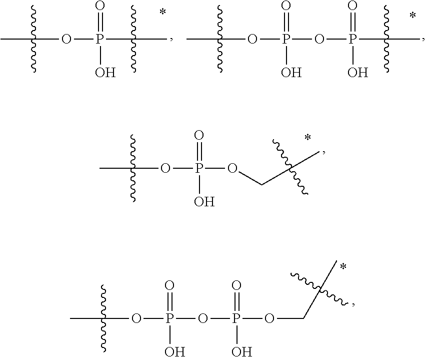 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

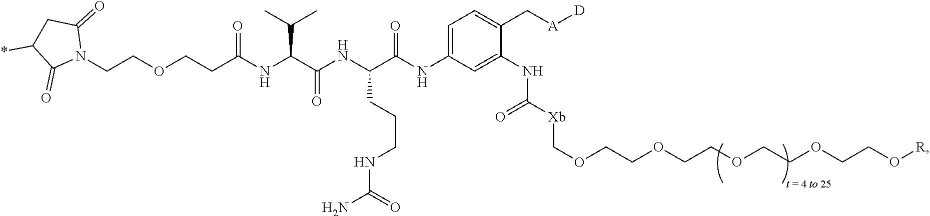

wherein: [symbol] is a bond to the antibody; and Xb, A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

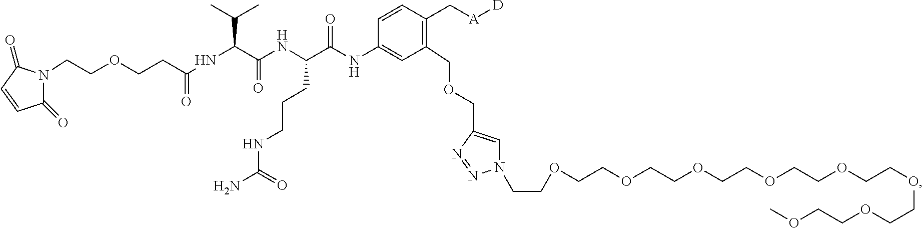

wherein:
A is a bond, —OC(═O)—*,

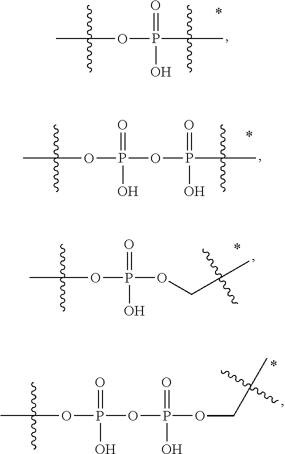 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

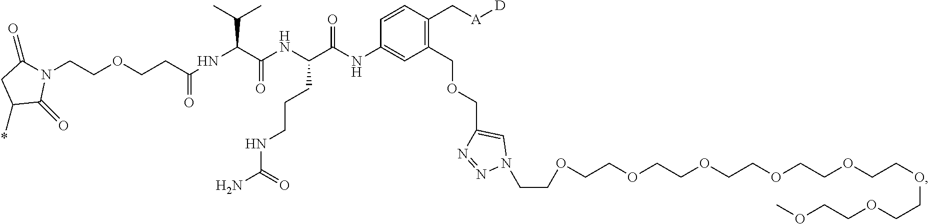

wherein: [symbol] is a bond to the antibody; and A and are as defined above. In some embodiments, A is a bond or —OC(═O)—*.

In some embodiments, the linker-drug group -(L-D) comprises or is formed from a compound of formula:

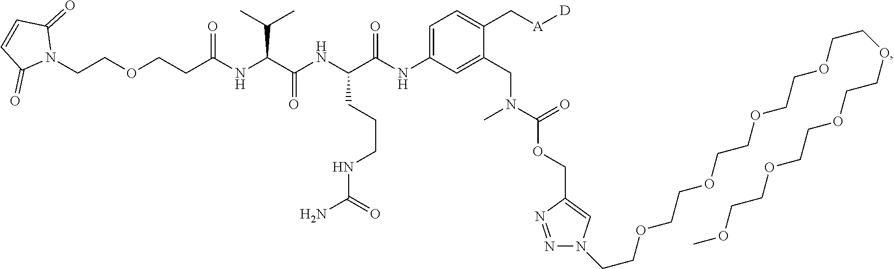

wherein:
A is a bond, —OC(═O)—*,

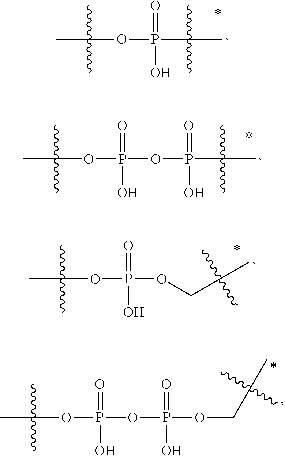 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(—O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

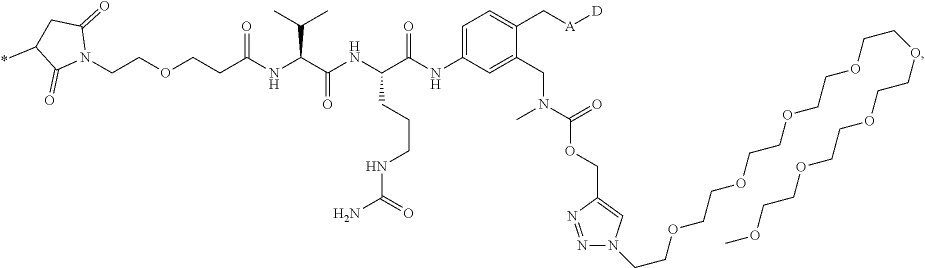

wherein: [symbol] is a bond to the antibody; and A and D are as defined above. In some embodiments, A is a bond or —OC(═O)—*.

In some embodiments, the linker-drug group -(L-D) comprises or is formed from a compound of formula:

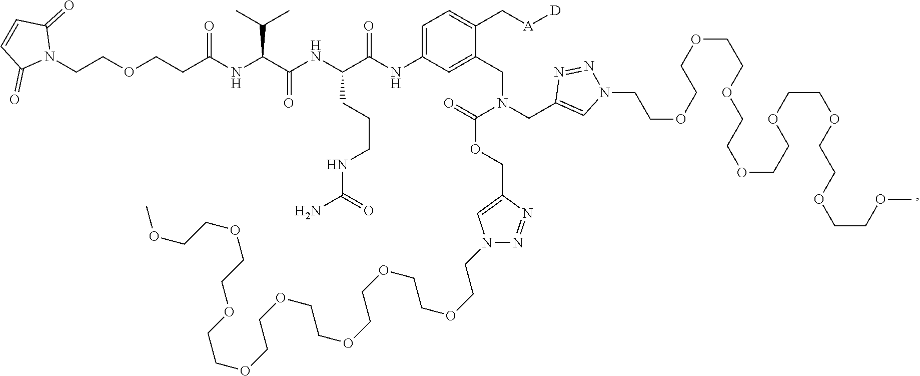

wherein:
A is a bond, —OC(—O)—*,

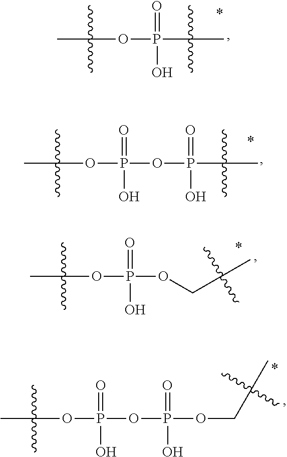

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

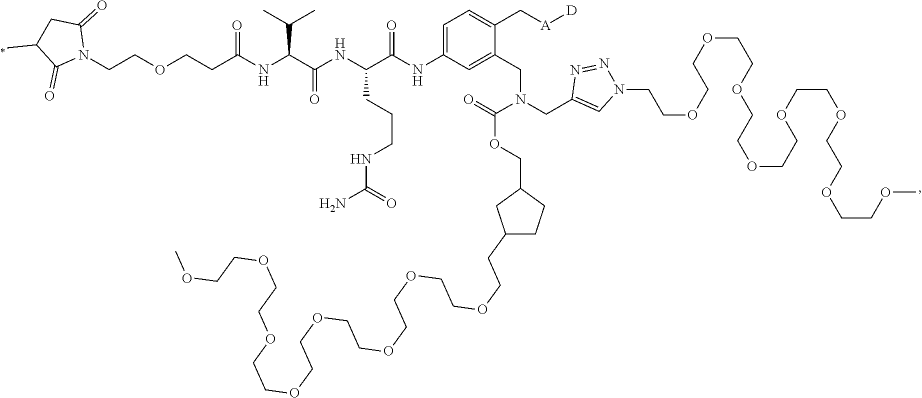

wherein: [symbol] is a bond to the antibody; and A and D are as defined above. In some embodiments, A is a bond or —OC(═O)—*.

In some embodiments, the linker-drug group -(L-D) comprises or is formed from a compound of formula:

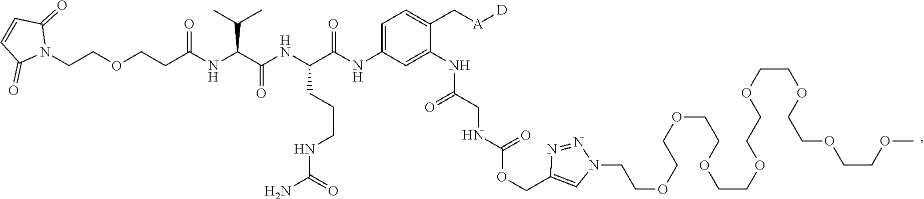

wherein:
A is a bond, —OC(═O)—*,

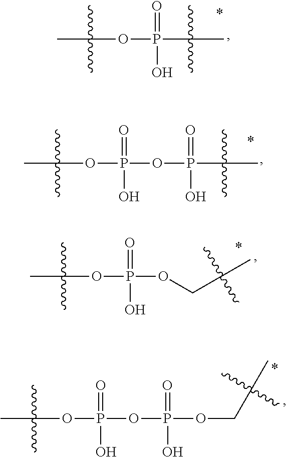

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

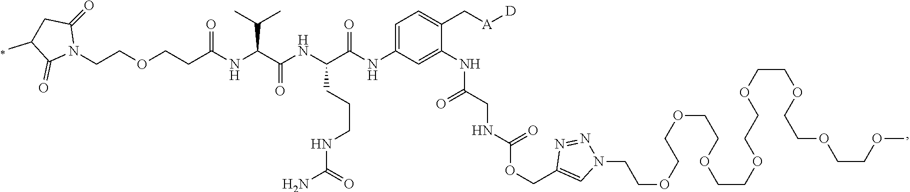

wherein: [symbol] is a bond to the antibody; and A and D are as defined above. In some embodiments, A is a bond or —OC(═O)—*.

In some embodiments, the linker-drug group -(L-D) comprises or is formed from a compound of formula:

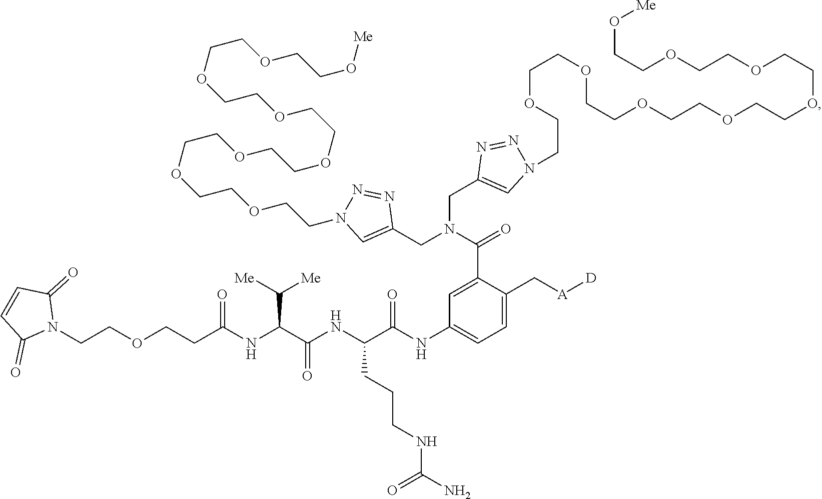

wherein:
A is a bond, —OC(═O)—*,

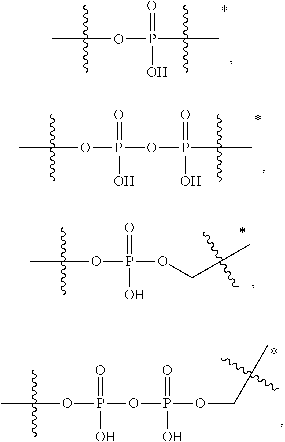 [SMILES: *OP(*)(=O)O ; *OP(=O)(O)OP(*)(=O)O ; *COP(=O)(O)O* ; *COP(=O)(O)OP(=O)(O)O*]

—OC(—O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

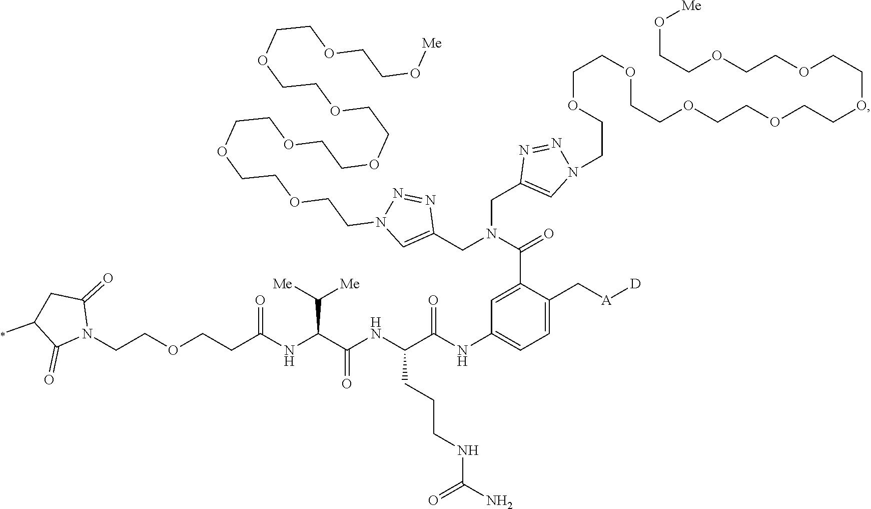

wherein: [symbol] is a bond to the antibody; and A and D are as defined above. In some embodiments, A is a bond or —OC(═O)—*.

In some embodiments, the linker-drug group -(L-D) comprises or is formed from a compound of formula:

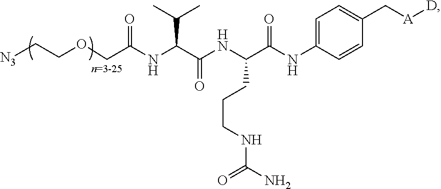

wherein:
A is a bond, —OC(═O)—*,

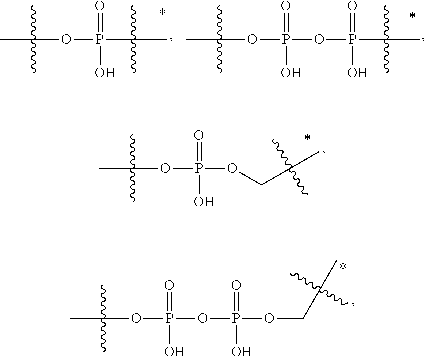 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(—O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

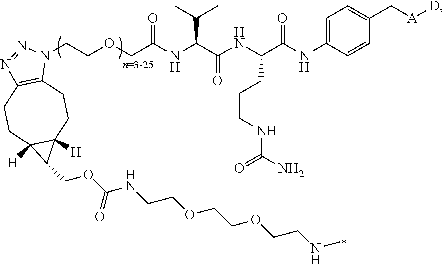

wherein: [symbol] is a bond to the antibody; and A and D are as defined above. In some embodiments, A is a bond or —OC(═O)—*.

In some embodiments, the linker-drug group -(L-D) comprises or is formed from a compound of formula:

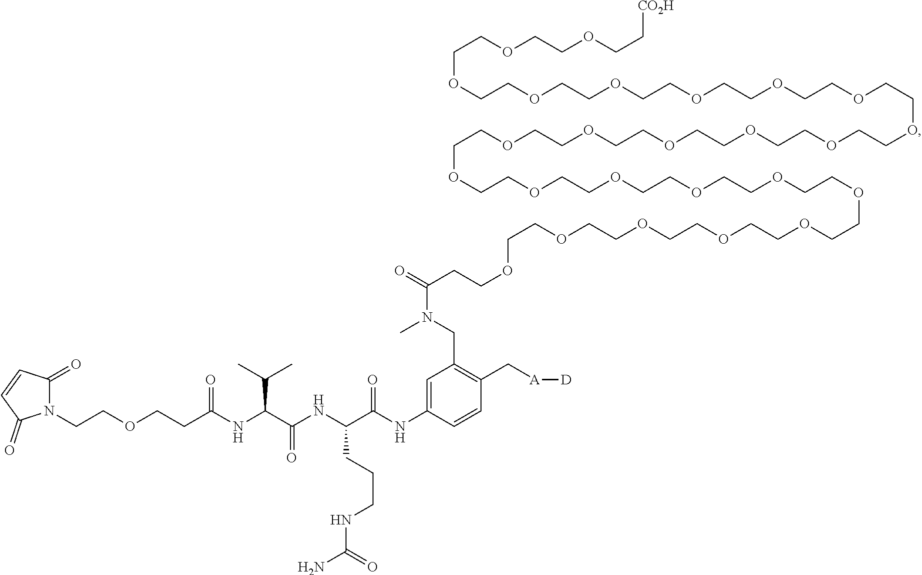

wherein:
A is a bond, —OC(═O)—*,

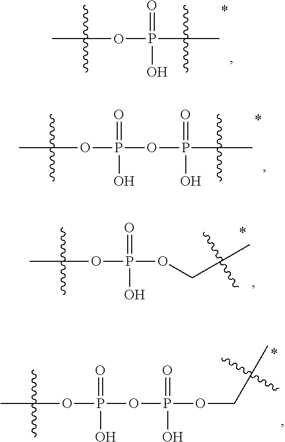 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

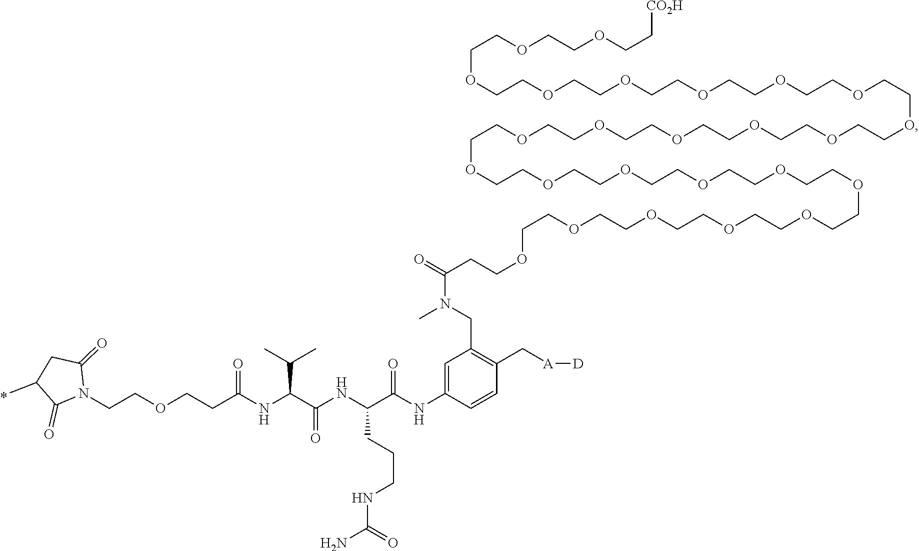

wherein: [symbol] is a bond to the antibody; and A and D are as defined above. In some embodiments, A is a bond or —OC(═O)—*.

In some embodiments, the linker-drug group -(L-D) comprises or is formed from a compound of formula:

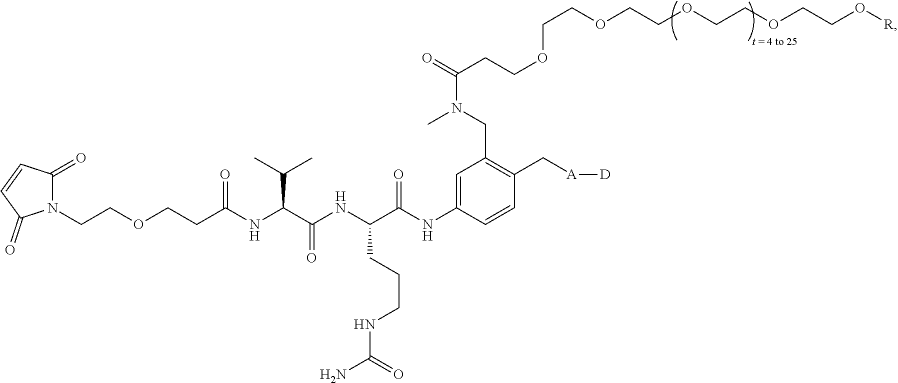

wherein:
each R independently is H, —CH₃ or —CH₂CH₂C(═O)OH;
A is a bond, —OC(═O)—*,

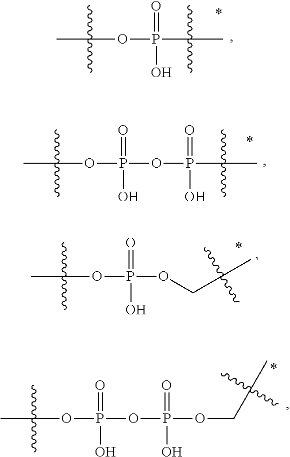 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor. In some embodiments, the linker-drug group -(L-D) comprises the following formula:

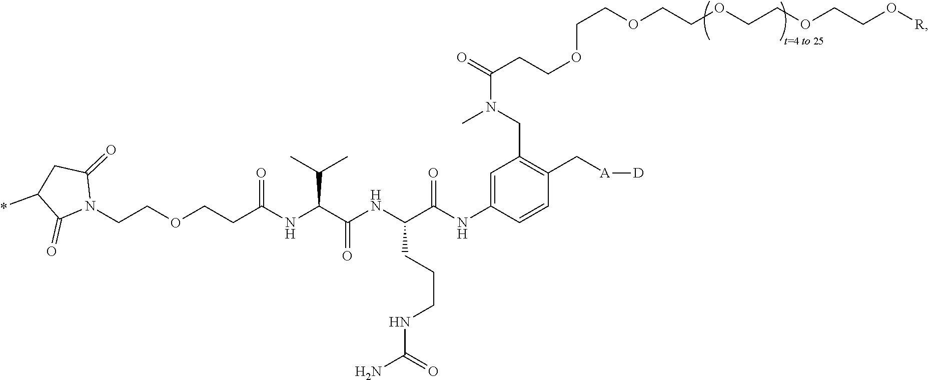

wherein: [symbol] is a bond to the antibody; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

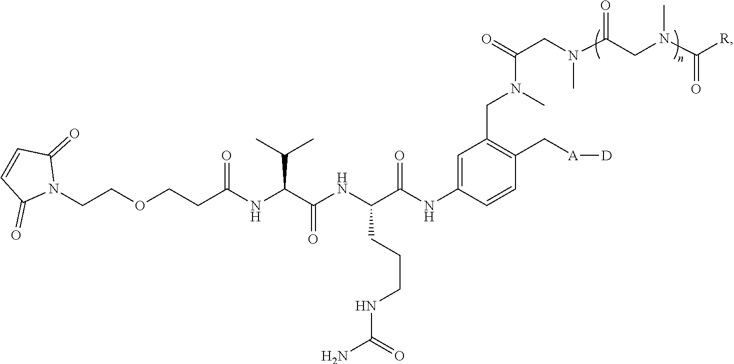

wherein:
each R independently is H, —CH₃ or —CH₂CH₂C(═O)OH;
A is a bond, —OC(═O)—*,

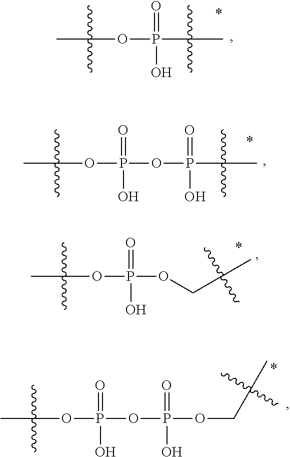 [SMILES: *OP(*)(=O)O ; *OP(=O)(O)OP(*)(=O)O ; *COP(=O)(O)O* ; *COP(=O)(O)OP(=O)(O)O*]

—OC(—O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
   D is a Bcl-xL inhibitor. In some embodiments, the linker-drug group -(L-D) comprises the following formula:

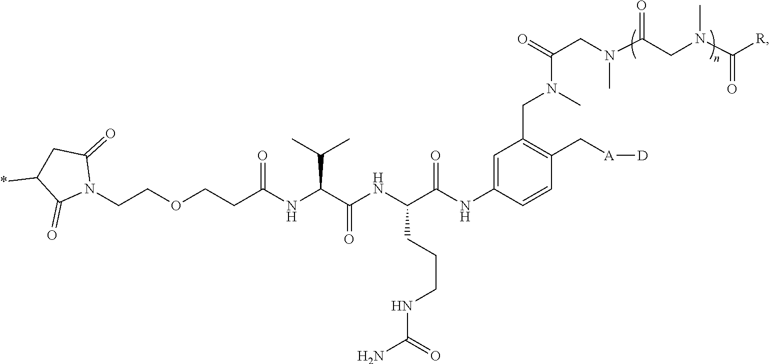

wherein: [symbol] is a bond to the antibody; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

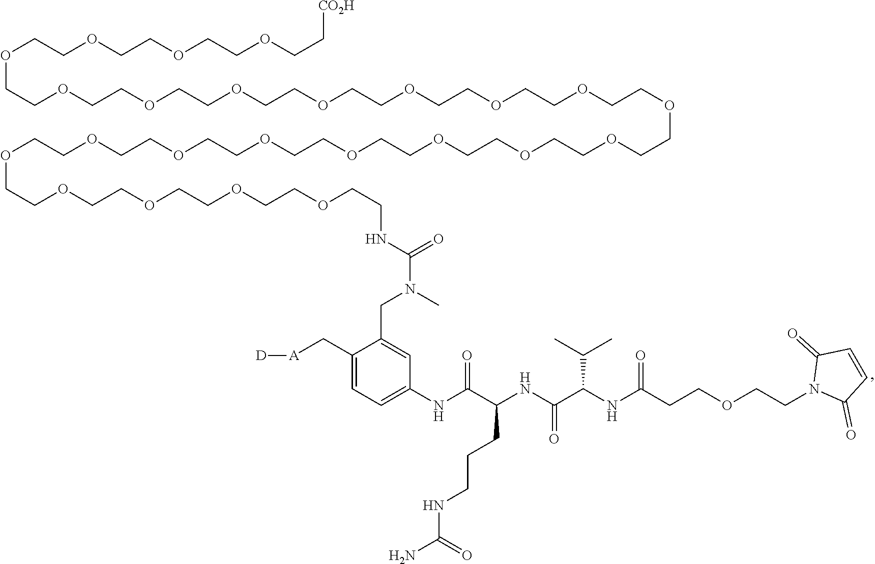

wherein:
A is a bond, —OC(—O)—*,

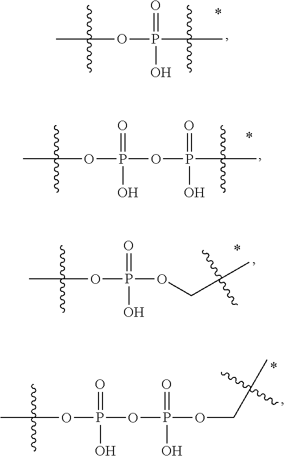 [SMILES: *OP(*)(=O)O ; *OP(=O)(O)OP(*)(=O)O ; *COP(=O)(O)O* ; *COP(=O)(O)OP(=O)(O)O*]

—OC(—O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor.

In some embodiments, A is a bond.

In some embodiments, A is —OC(—O)—*.

In some embodiments, R is —CH₃.

In some embodiments, R is —CH₂CH₂COOH.

In some embodiments, the antibody-drug conjugate comprises the linker-drug group, -(L-D), which is formed from a compound selected from:

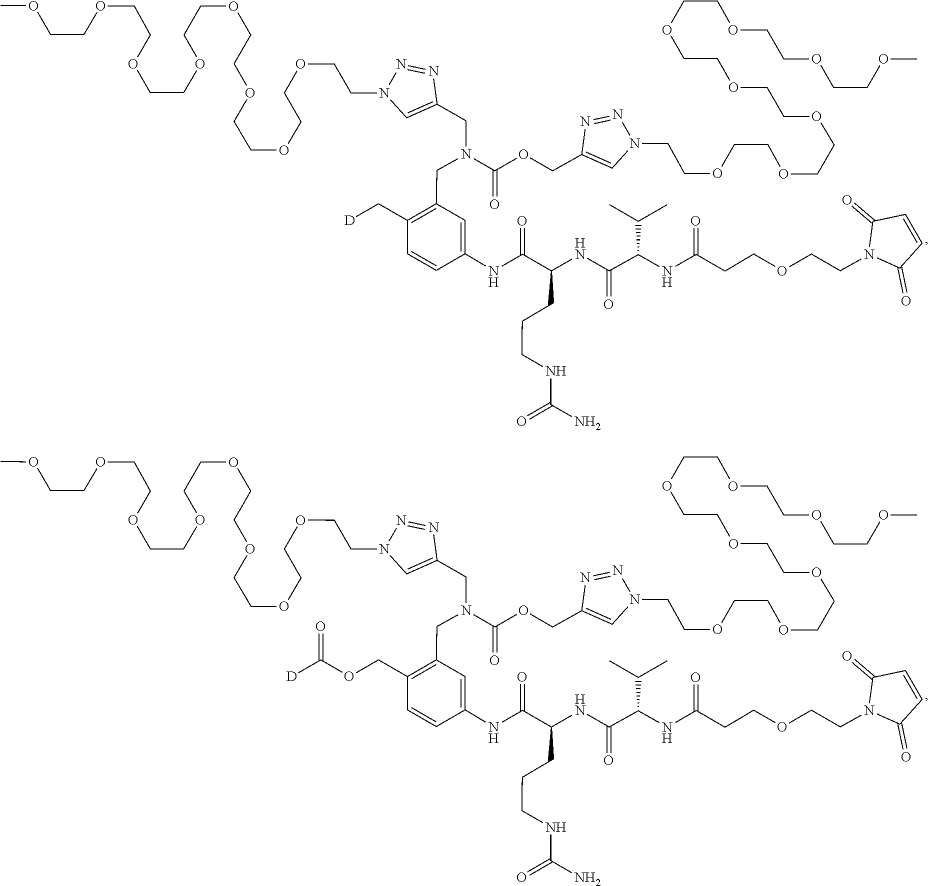 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOC)nn1)C(=O)OCc1cn(CCOCCOCCOCCOCCOCCOCCOCCOC)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOC)nn1)C(=O)OCc1cn(CCOCCOCCOCCOCCOCCOCCOCCOC)nn1]

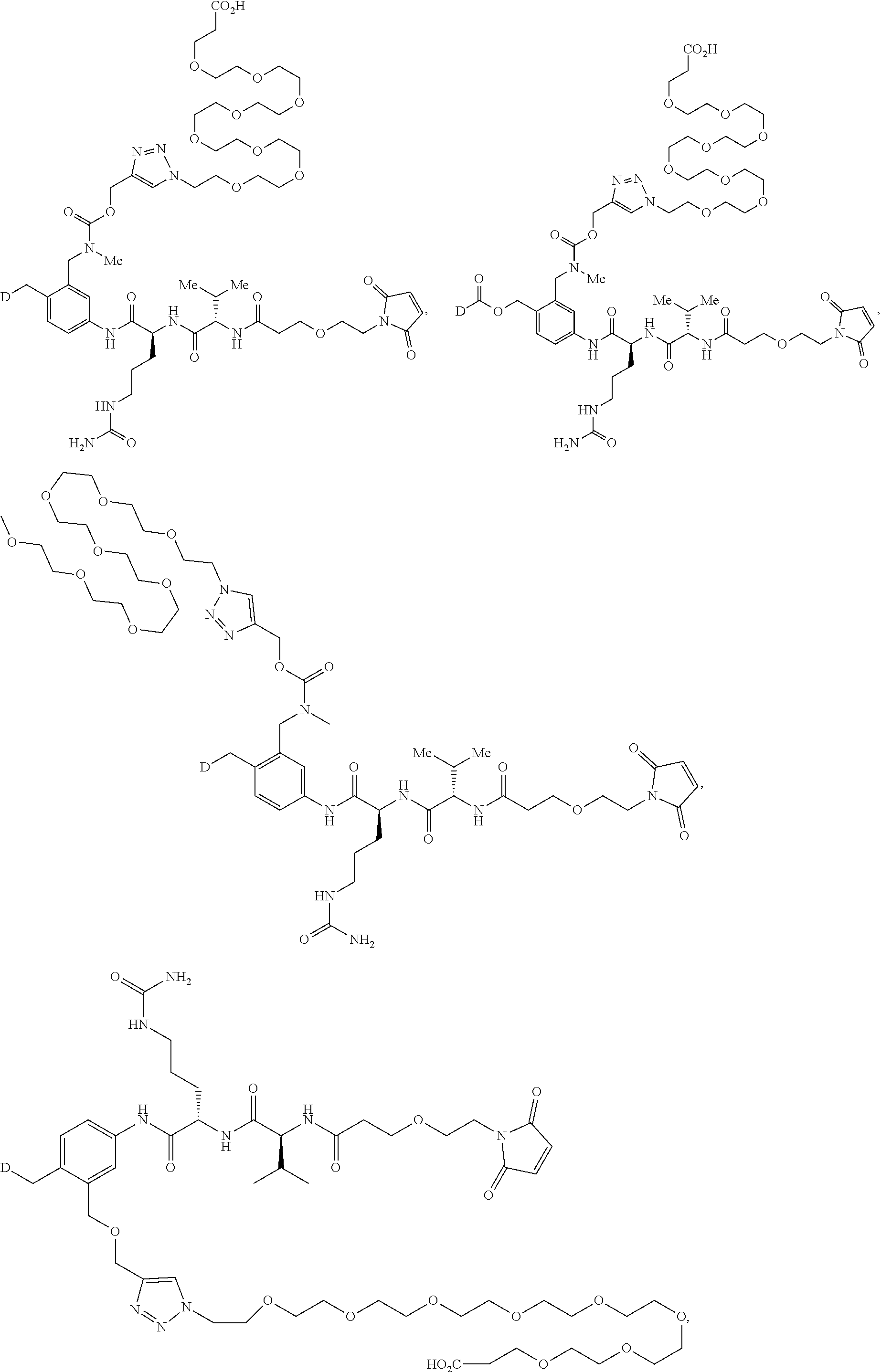

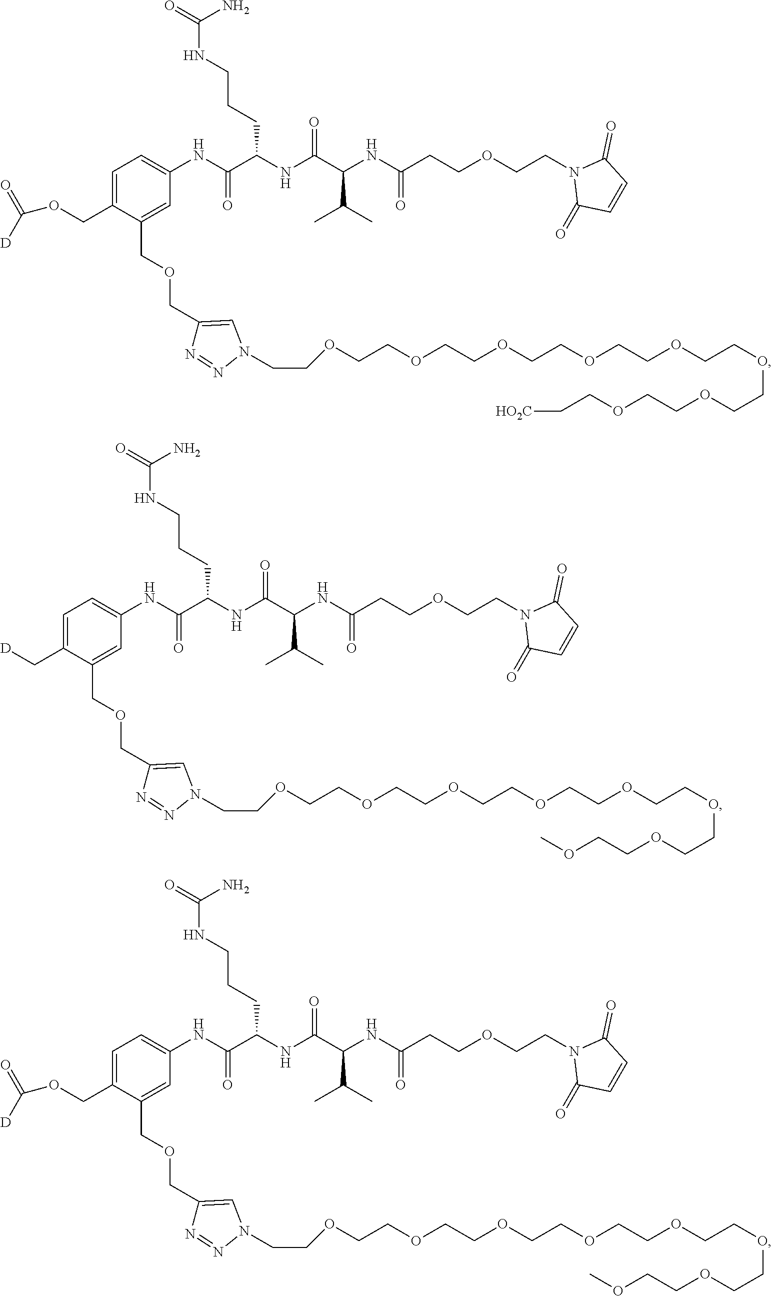 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOCCC(=O)O)nn1 ; [2H]Cc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOC)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOC)nn1]

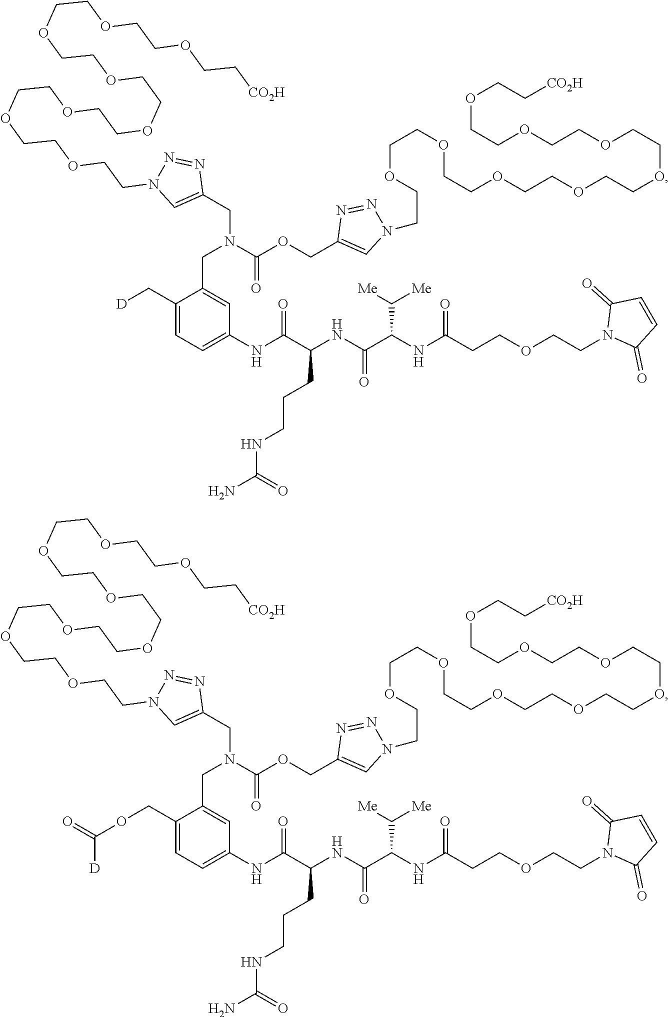 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCC(=O)O)nn1)C(=O)OCc1cn(CCOCCOCCOCCOCCOCCOCCOCCOCCC(=O)O)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCC(=O)O)nn1)C(=O)OCc1cn(CCOCCOCCOCCOCCOCCOCCOCCOCCC(=O)O)nn1]

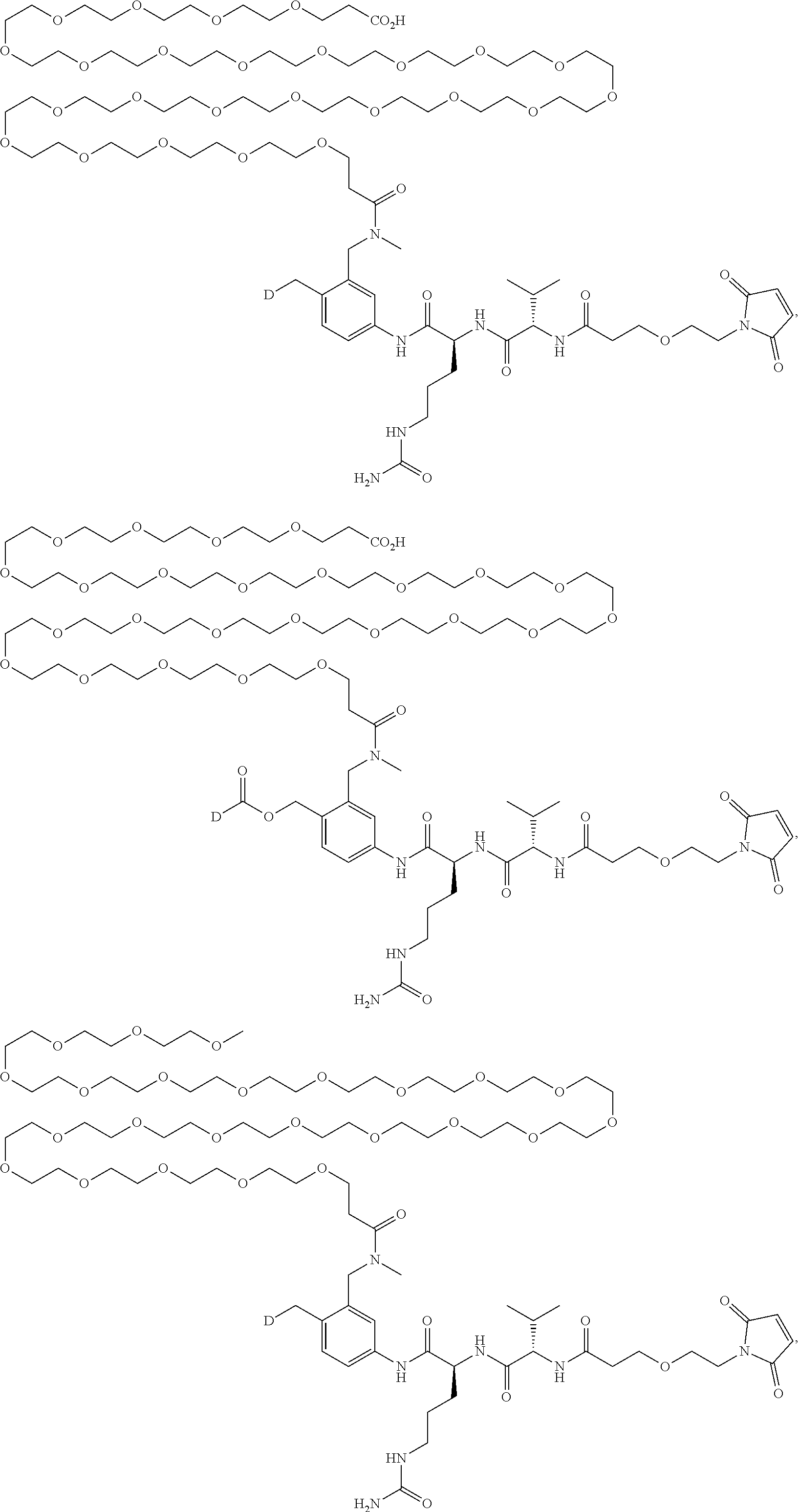

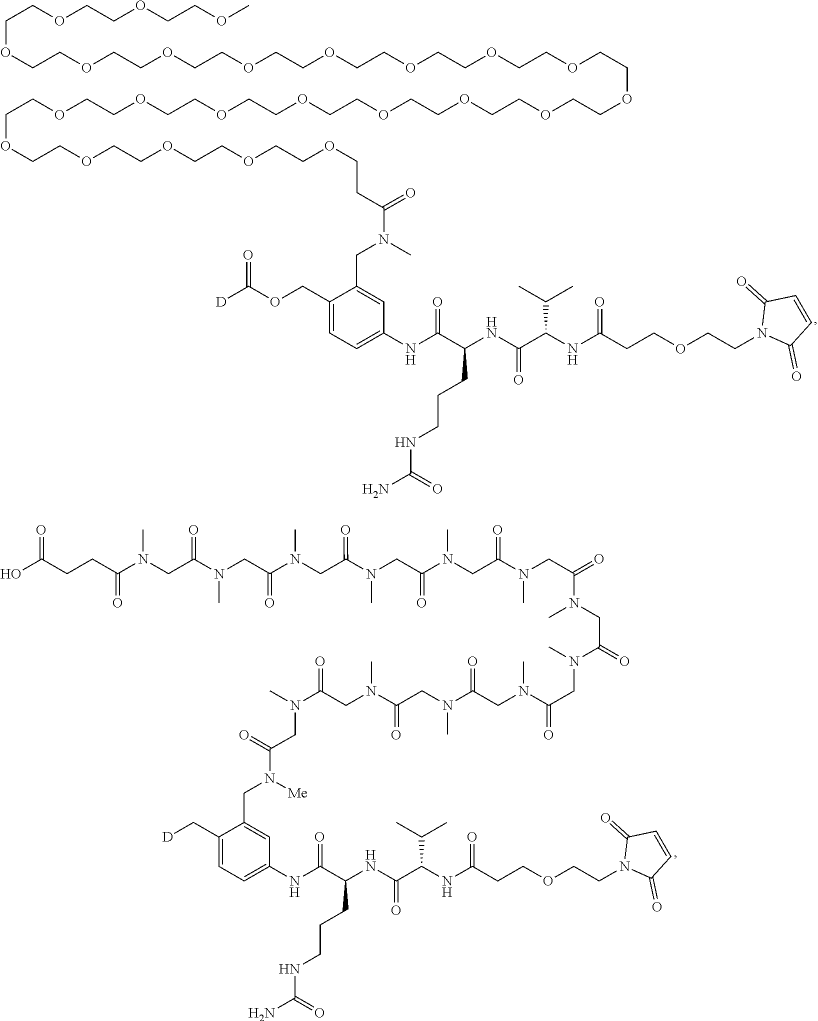

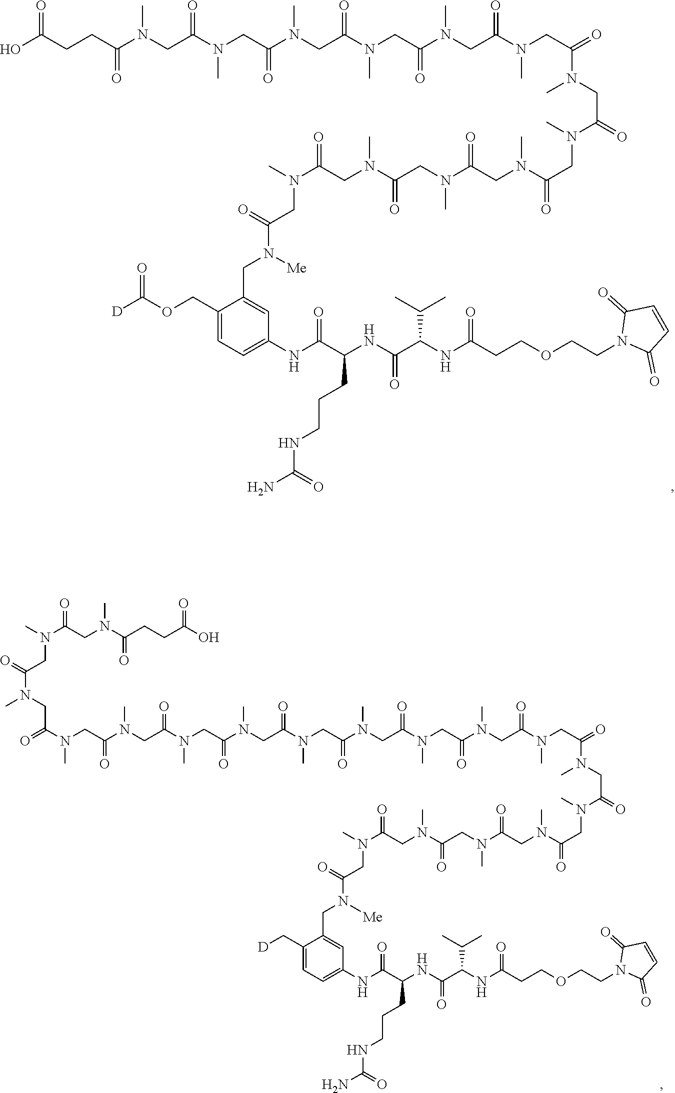

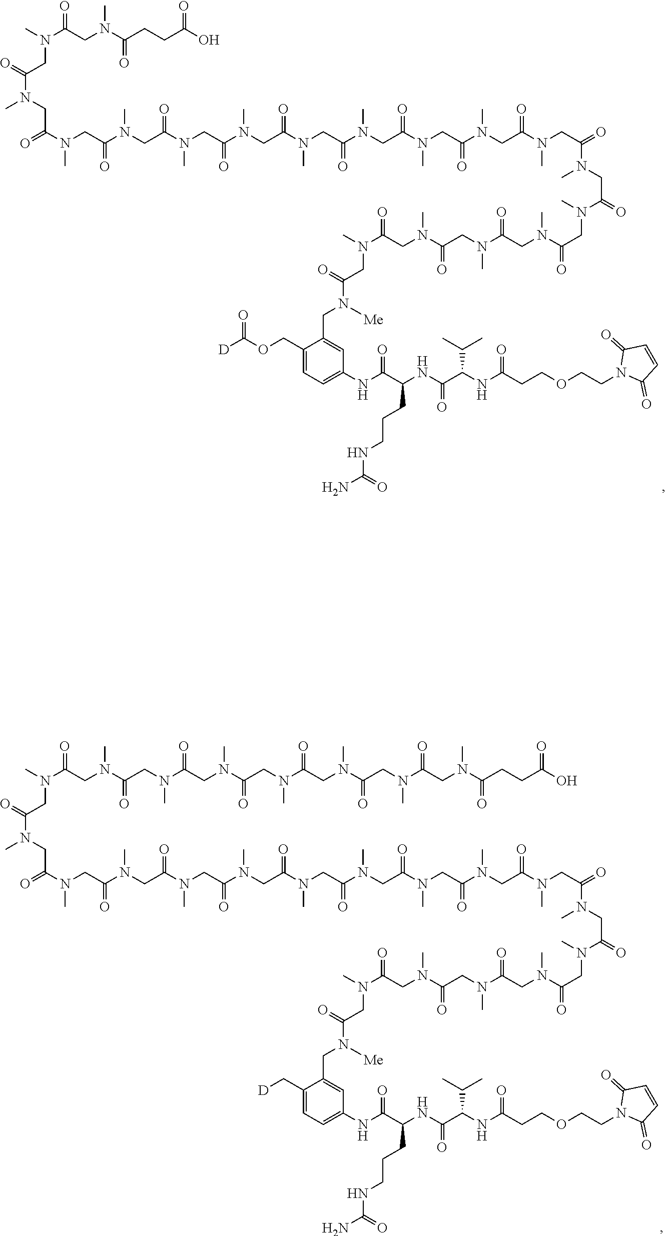

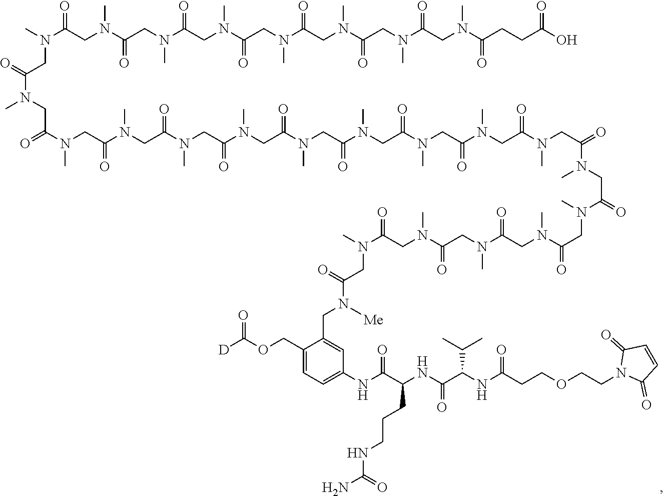 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O]

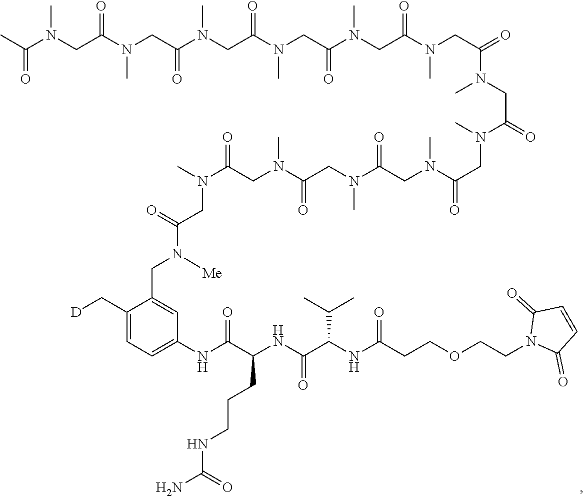 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O]

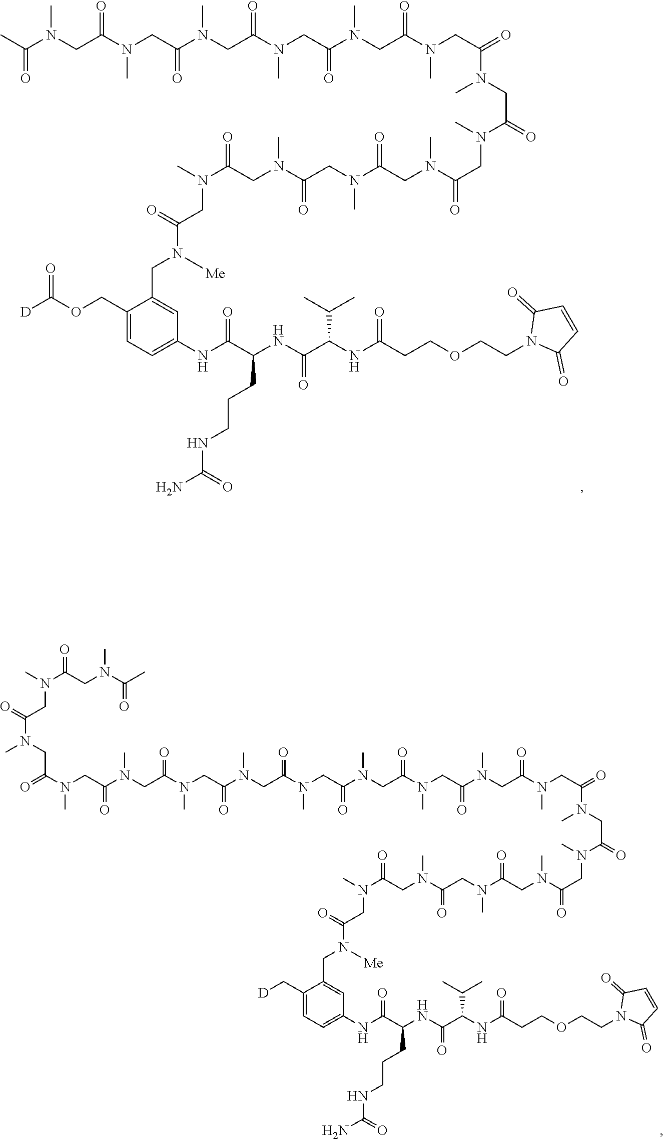 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O]

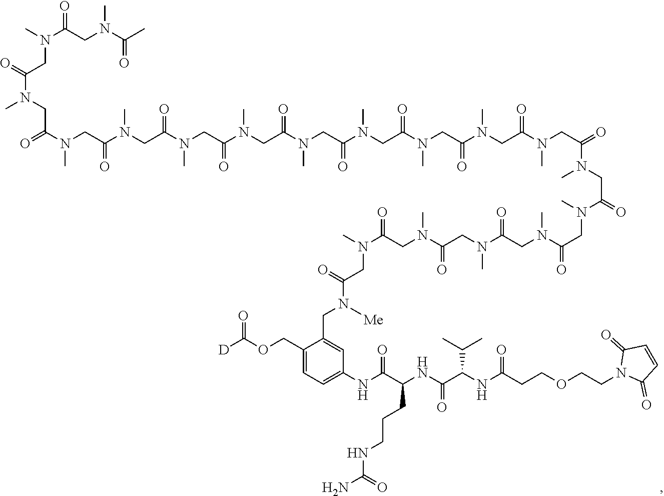 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O]

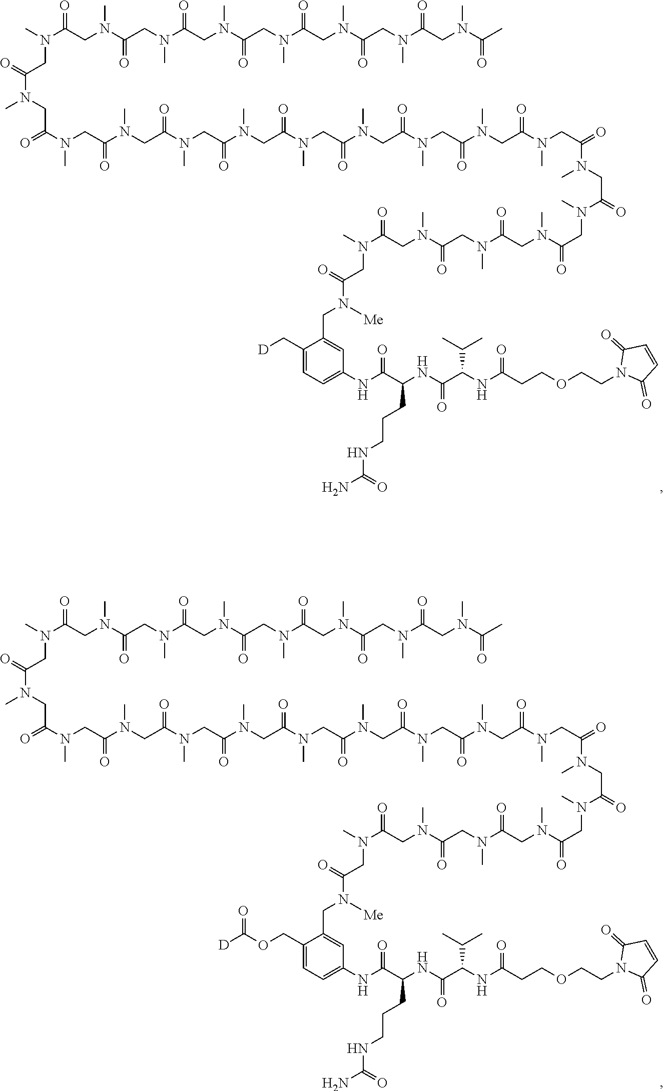

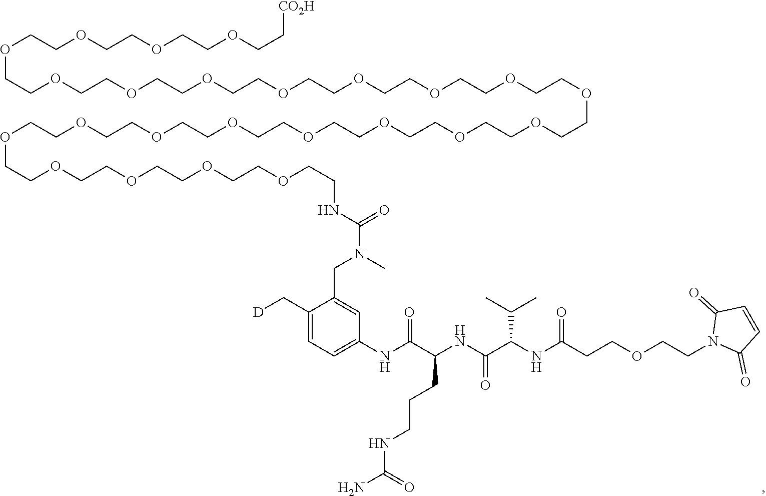 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O]

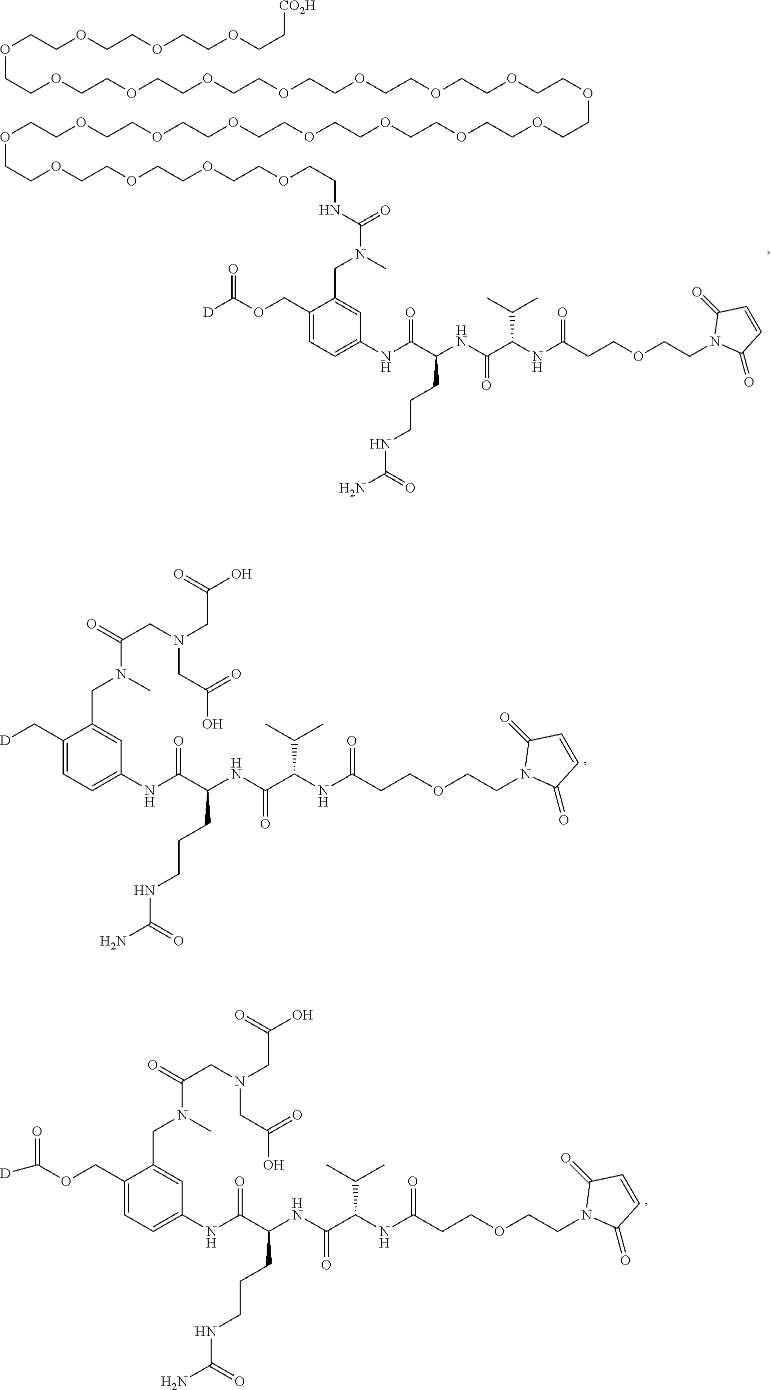 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O ; [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(CC(=O)O)CC(=O)O ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(CC(=O)O)CC(=O)O]

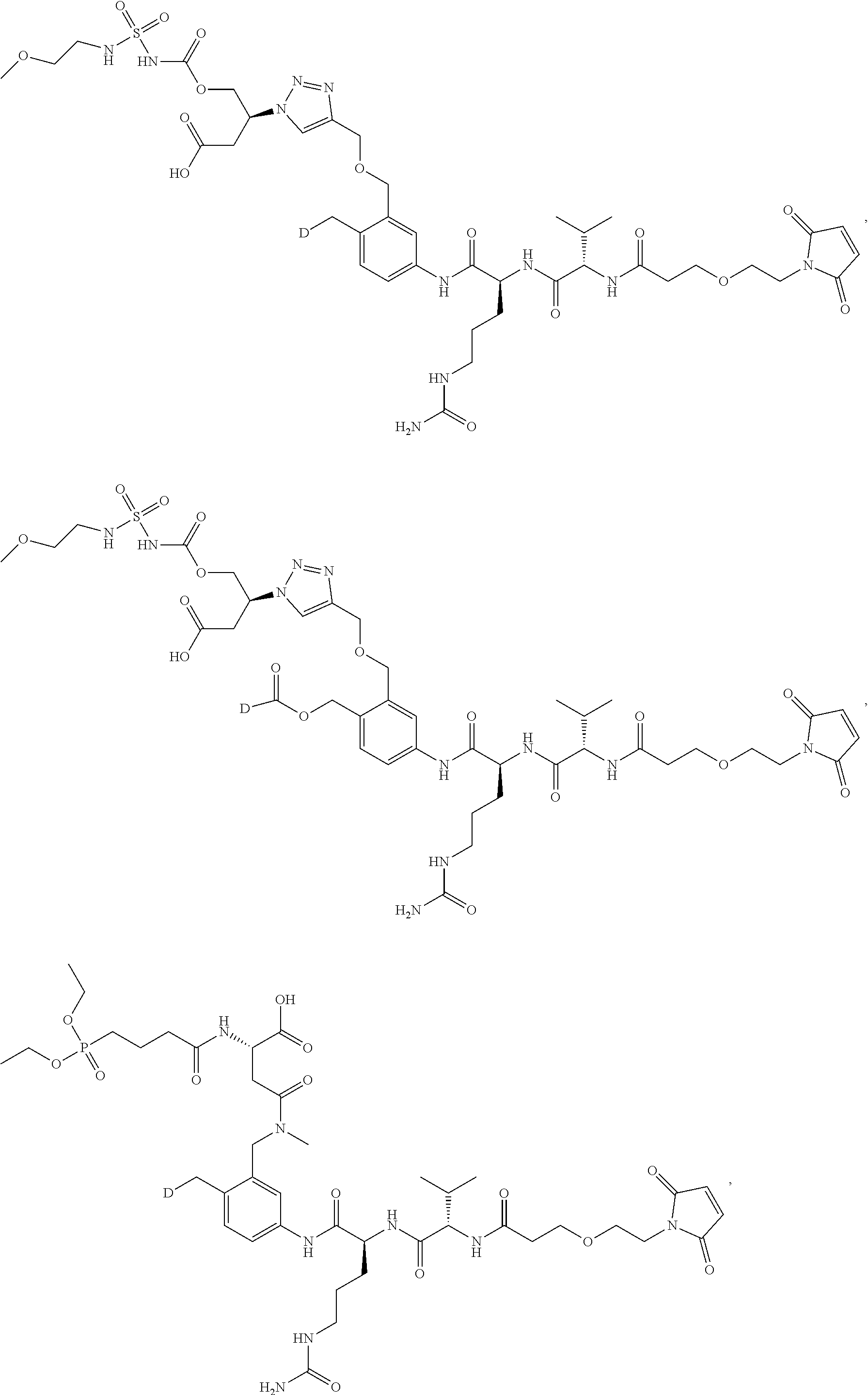 [SMILES: [2H]Cc1ccc(NC(=O)[C@H](CCCNC(N)=O)NC(=O)[C@@H](NC(=O)CCOCCN2C(=O)C=CC2=O)C(C)C)cc1COCc1cn([C@H](COC(=O)NS(=O)(=O)NCCOC)CC(=O)O)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1COCc1cn([C@H](COC(=O)NS(=O)(=O)NCCOC)CC(=O)O)nn1 ; [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)CCCP(=O)(OCC)OCC)C(=O)O]

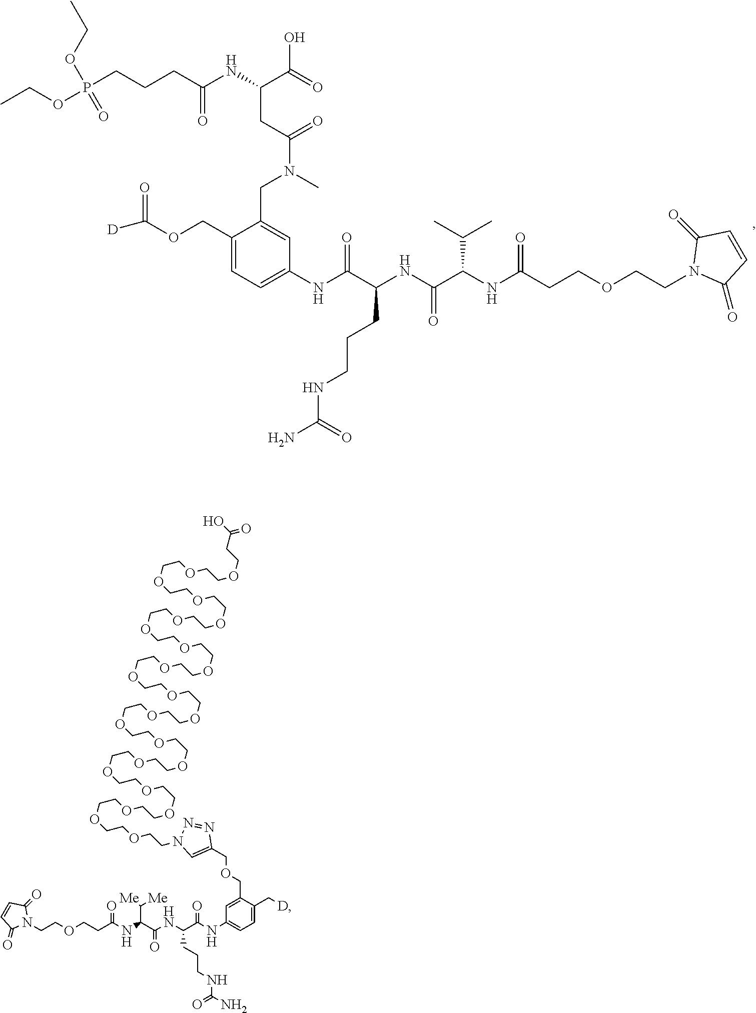

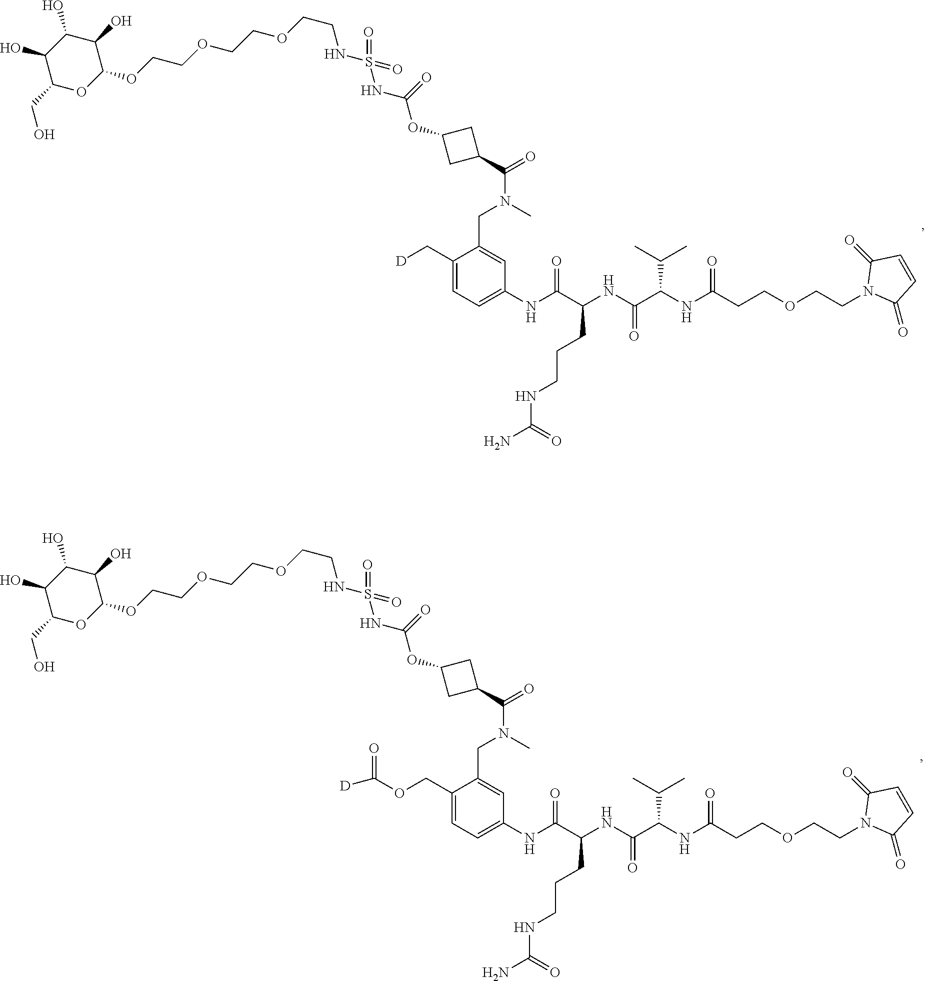 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@H]1C[C@H](OC(=O)NS(=O)(=O)NCCOCCOCCO[C@@H]2O[C@H](CO)[C@@H](O)[C@H](O)[C@H]2O)C1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@H]1C[C@H](OC(=O)NS(=O)(=O)NCCOCCOCCO[C@@H]2O[C@H](CO)[C@@H](O)[C@H](O)[C@H]2O)C1]

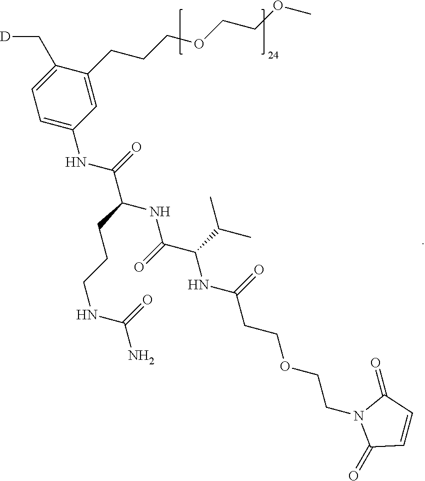

In some embodiments, the antibody-drug conjugate comprises the linker-drug group, -(L-D), which comprises a formula selected from:

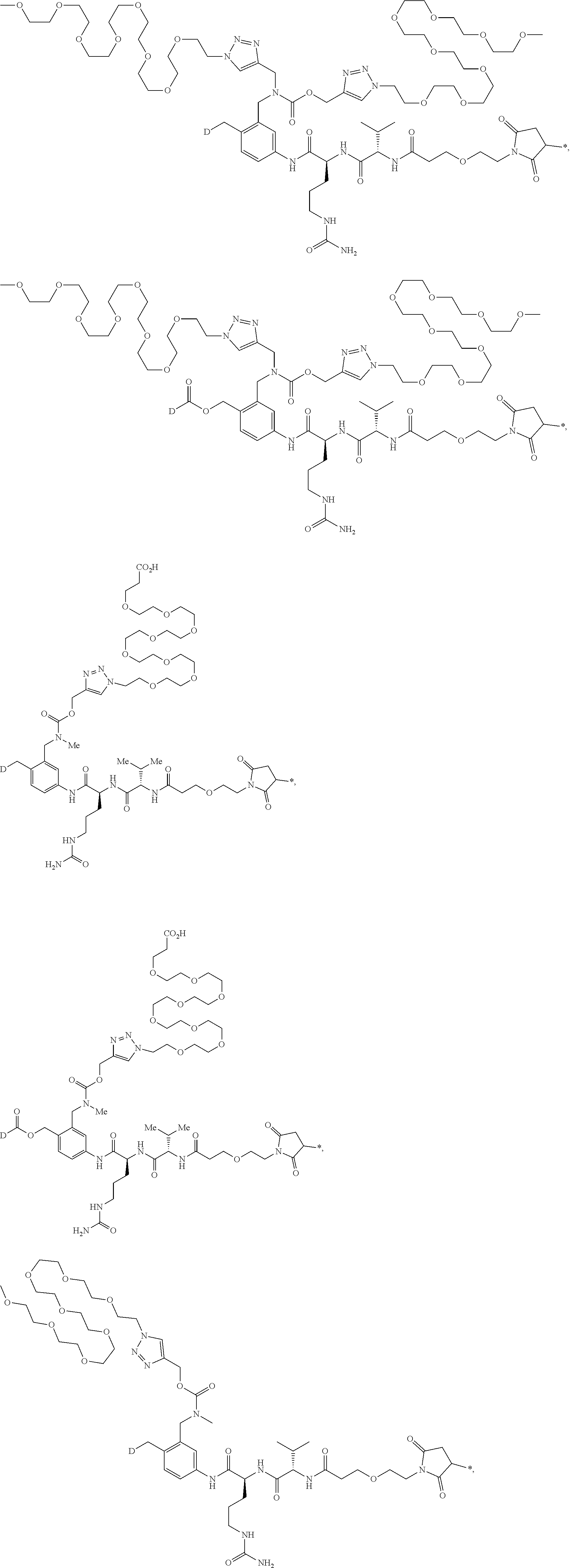

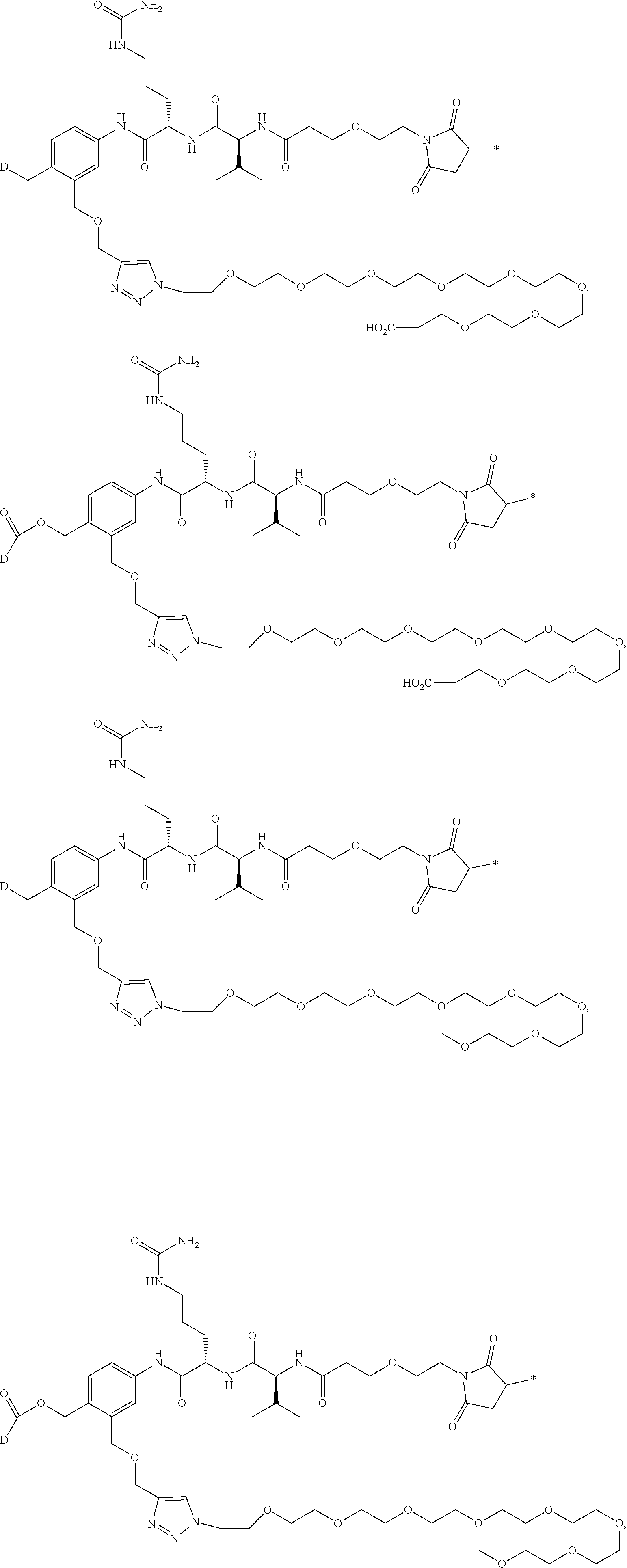

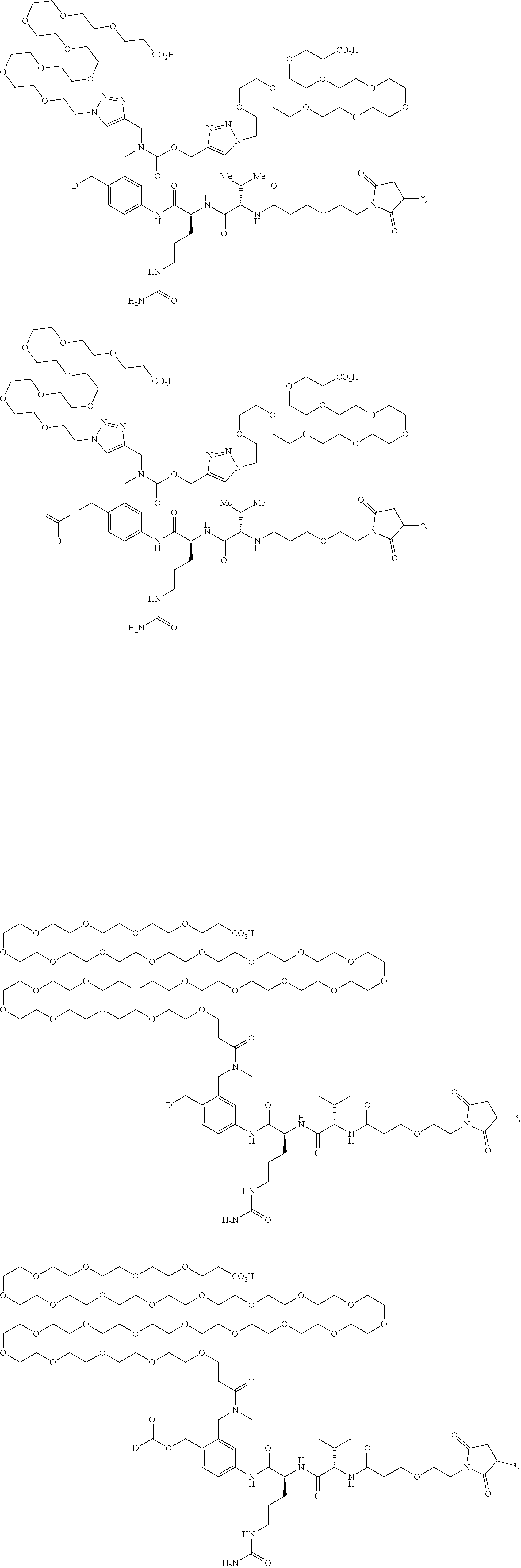

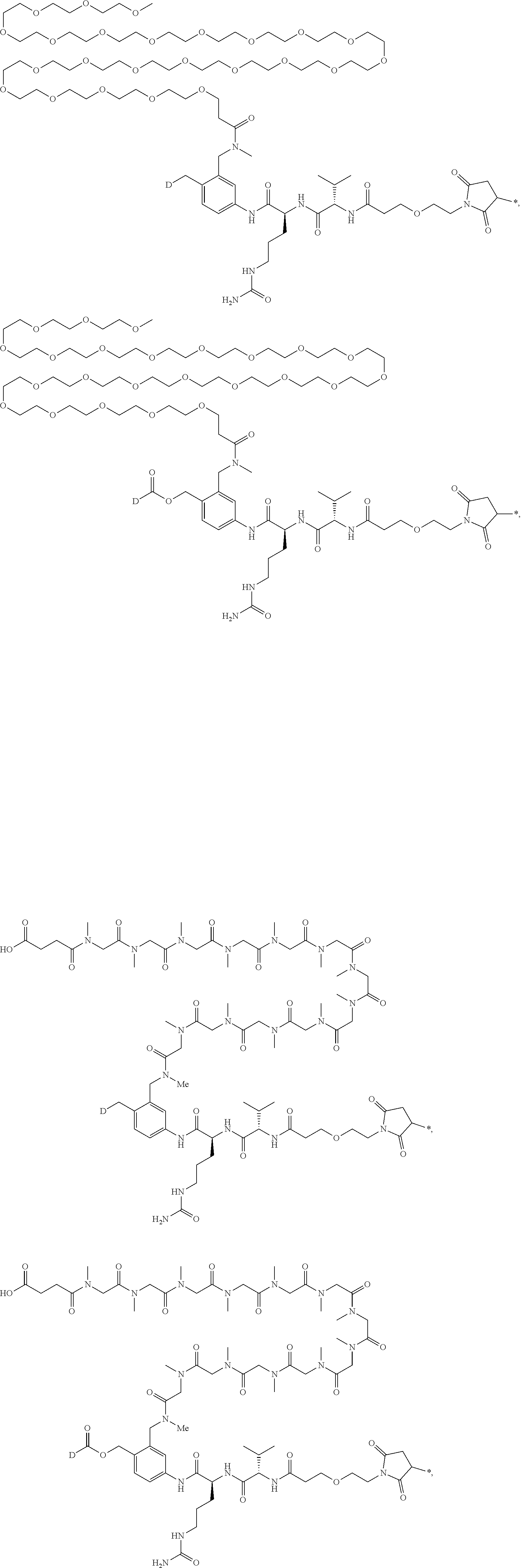

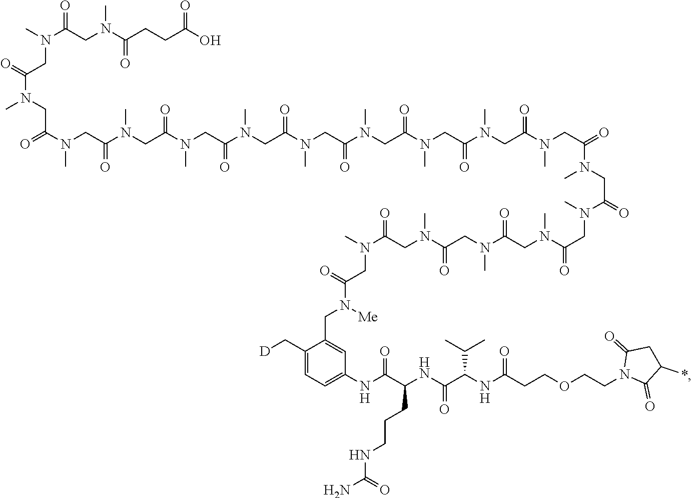

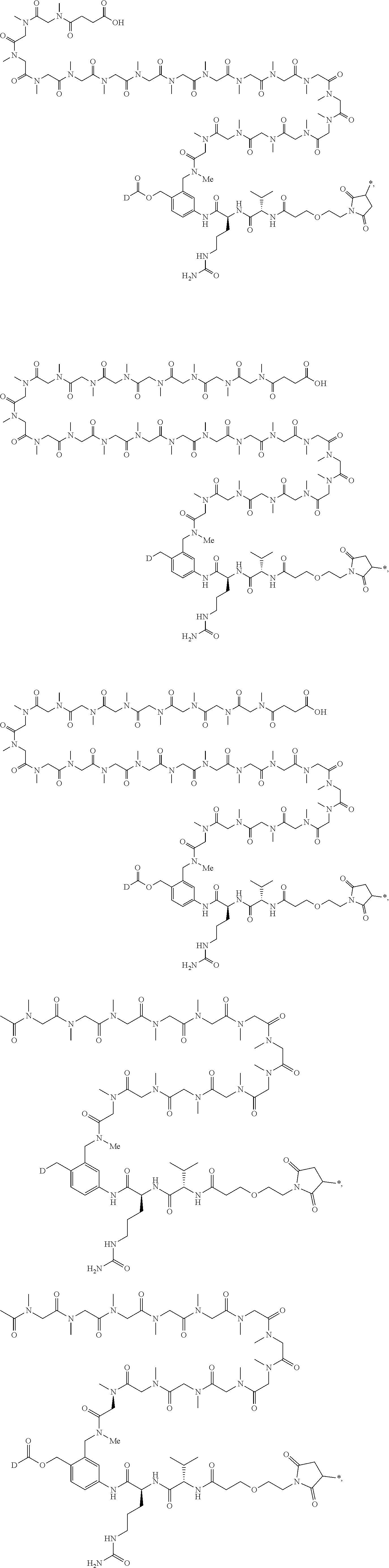

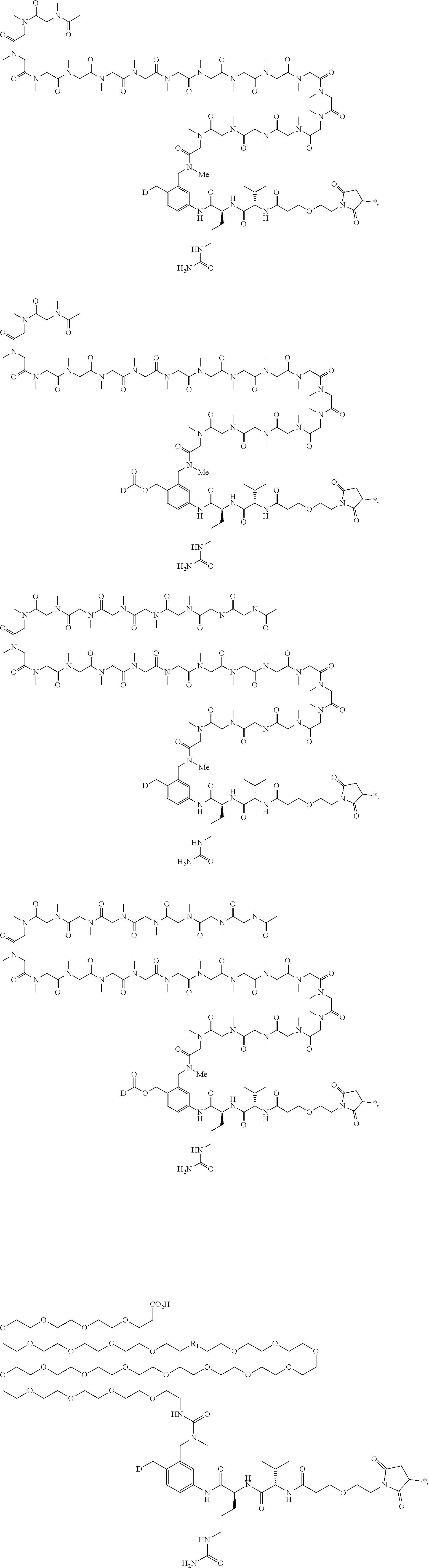

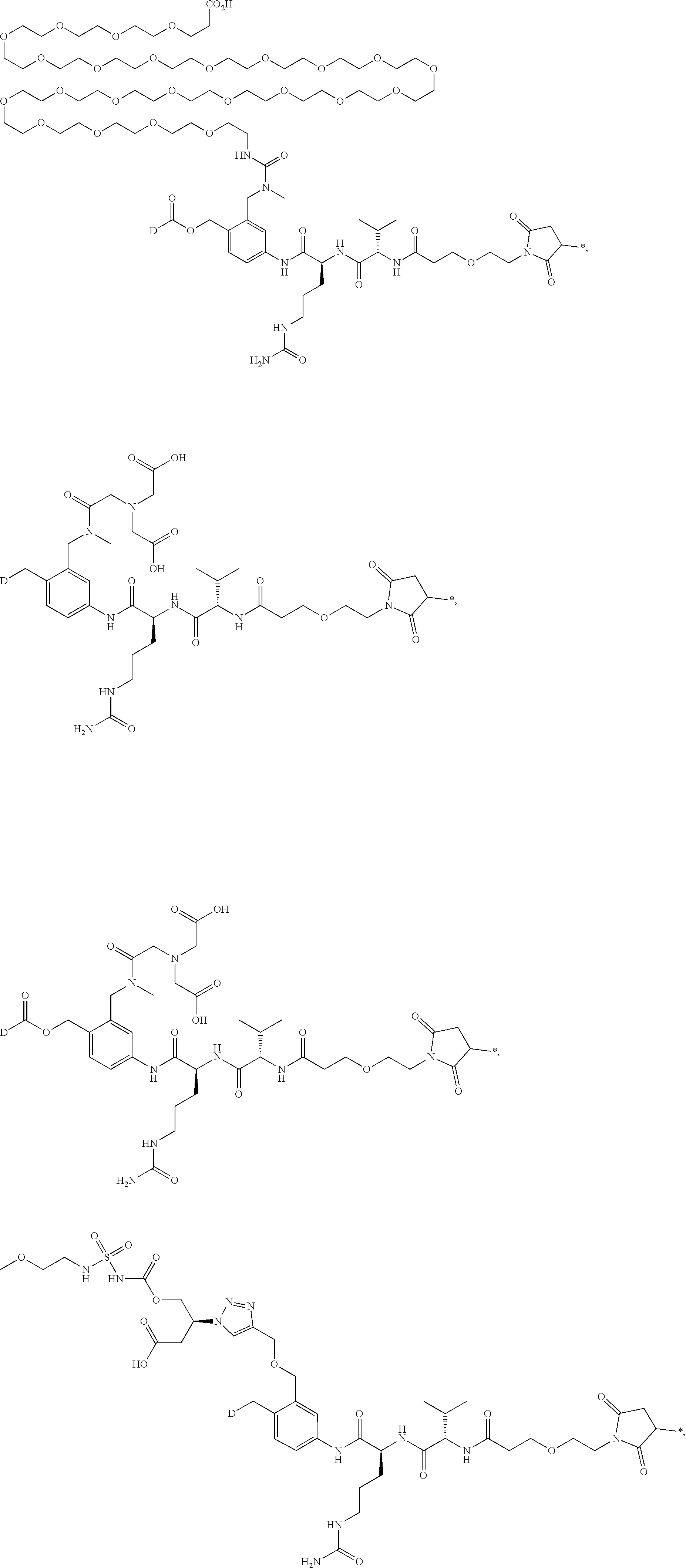

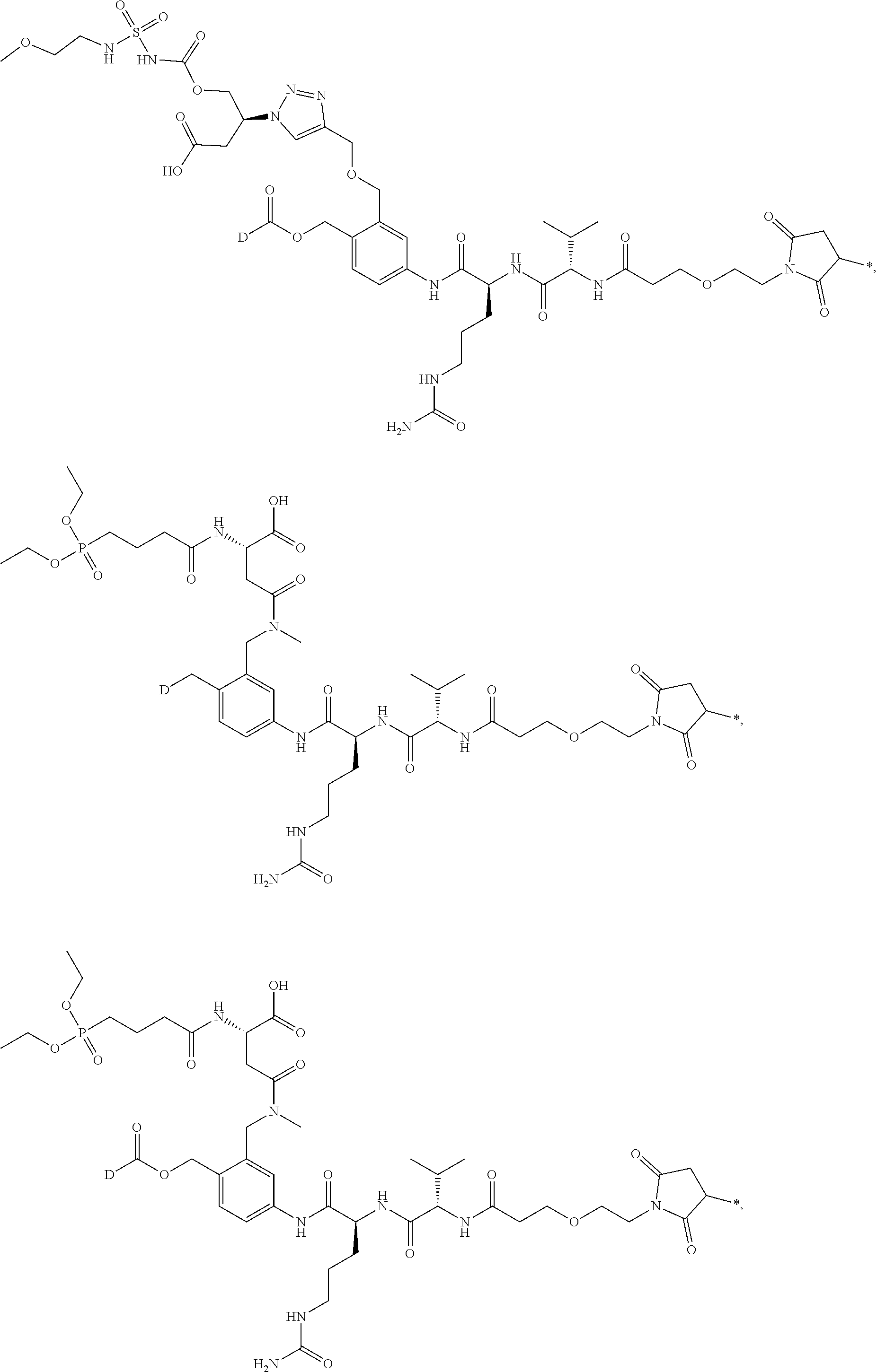

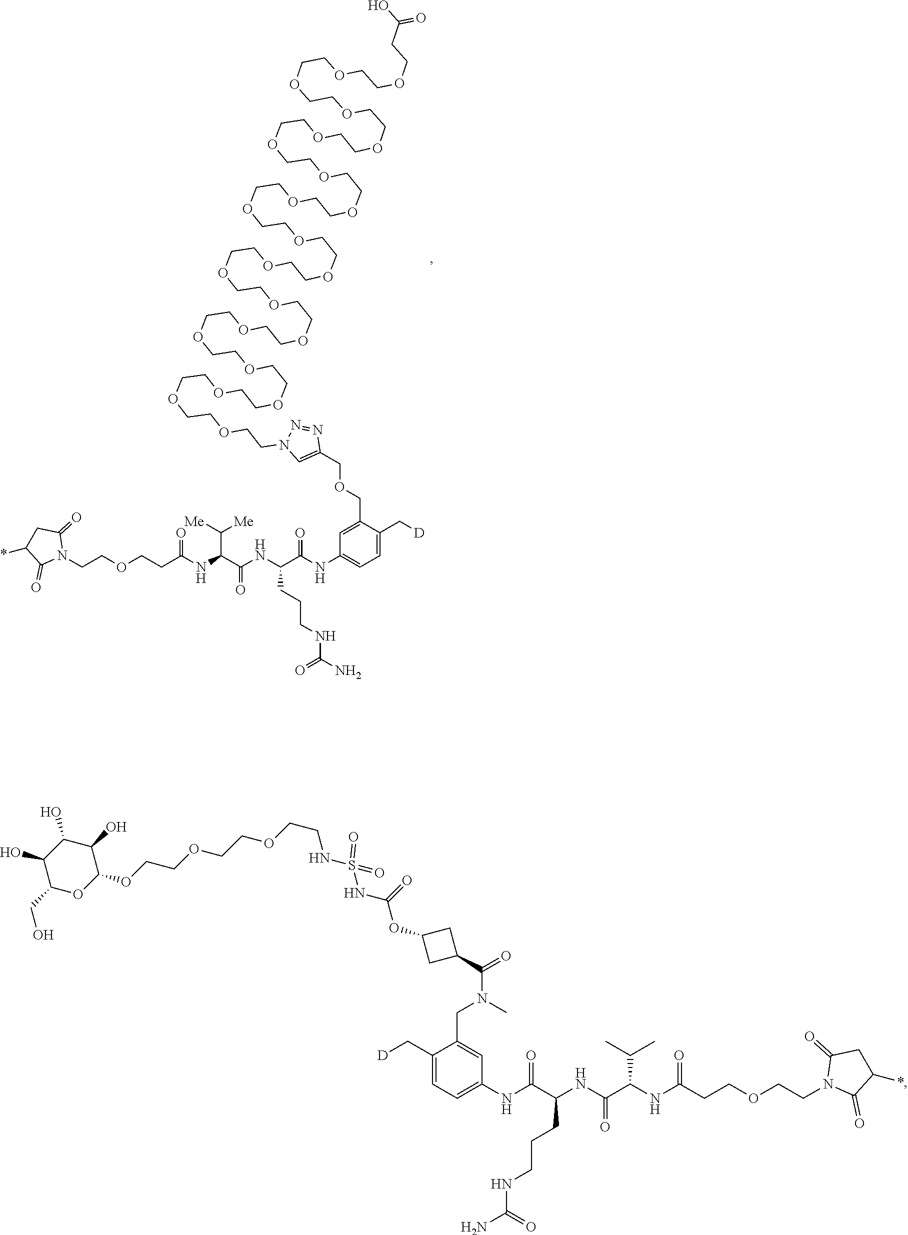

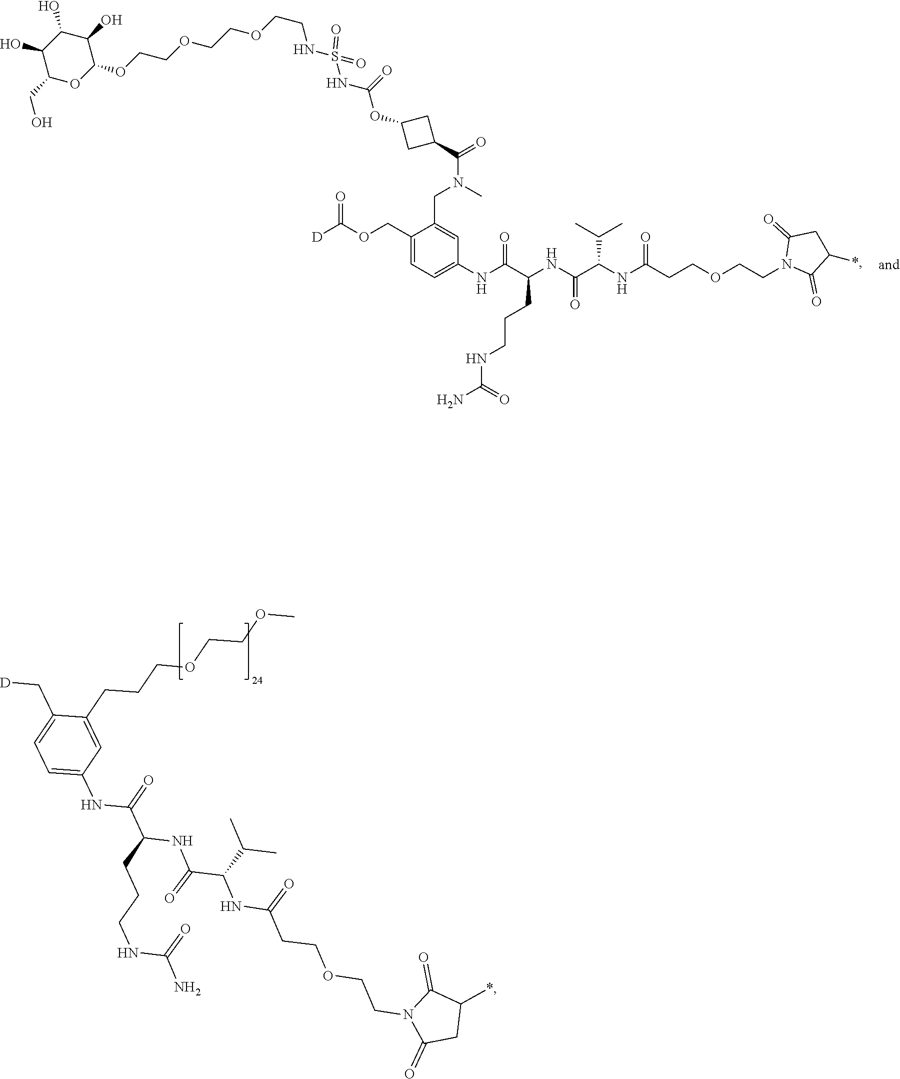

and wherein [symbol] is a bond to the antibody.

In some embodiments, the Bcl-xL inhibitor (D) comprises a compound of Formula (I):

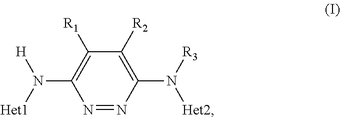

or an enantiomer, a diastereoisomer, and/or a pharmaceutically acceptable salt of any one of the foregoing, wherein the variables are described above for Formula (I). In some embodiments, R1 is linear or branched C1-6alkyl and R2 is H.

In some embodiments, the Bcl-xL inhibitor (D) comprises a compound of Formula (II):

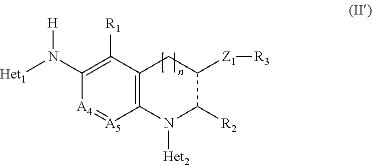

or an enantiomer, a diastereoisomer, and/or a pharmaceutically acceptable salt of any one of the foregoing, wherein the variables are described above for Formula (II). A1 and A5 both represent a nitrogen atom, R1 is linear or branched C1-6alkyl; R2 is H; n is 1; and [symbol] represents a single bond.

In some embodiments, the Bcl-xL inhibitor (D) comprises a compound of Formula (IA) or (IIA):

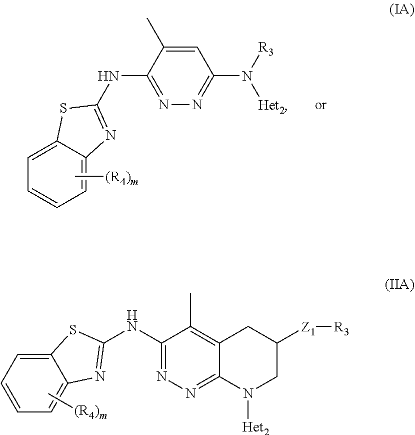

or an enantiomer, a diastereoisomer, and/or a pharmaceutically acceptable salt of any one of the foregoing, wherein:
Z₁ represents a bond or —O—,
R₃ represents a group selected from: hydrogen; C₃-C₆cycloalkyl; linear or branched C₁-C₆alkyl; —X₁—NRₐR_{b}; —X₁—N⁺RₐR_{b}R_{c}; and —X₁—O—R_{c},
Rₐ and R_{b} independently of one another represent a group selected from: hydrogen; linear or branched C₁-C₆alkyl optionally substituted by one or two hydroxyl groups; and C₁-C₆alkylene-SO₂O,
R_{c} represents a hydrogen or a linear or branched C₁-C₆alkyl group,
Het₂ represents a group selected from:

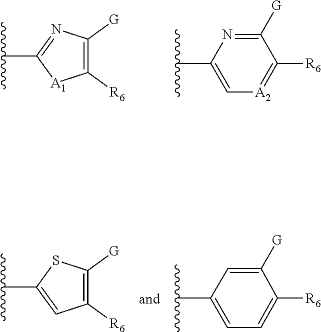

A₁ is —NH—, —N(C₁-C₃alkyl), O, S or Se,
A₂ is N, CH or C(R₅),
G is selected from the group consisting of:
—C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, C₁-C₆alkyl optionally substituted by a hydroxyl group, halogen, —NO₂, and —CN, in which:
   R_{G1} and R_{G2} at each occurrence are each independently selected from the group consisting of hydrogen, and C₁-C₆alkyl optionally substituted by 1 to 3 halogen atoms;
   R_{G3} is C₁-C₆alkyl optionally substituted by 1 to 3 halogen atoms; or
R_{G1} and R_{G2}, together with the atom to which each is attached are combined to form a C₃-C₈heterocycloalkyl;
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; halogen or —CN,
R₆ represents a group selected from:
—X₂—O—R₇; and
an heteroarylene-R₇ group optionally substituted by a linear or branched C₁-C₆alkyl group,
R₇ represents a group selected from: linear or branched C₁-C₆alkyl group;
(C₃-C₆) cycloalkylene-R₈; or:

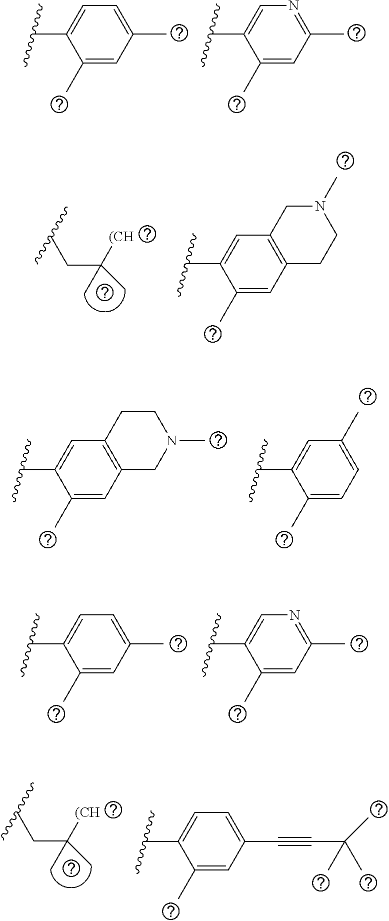

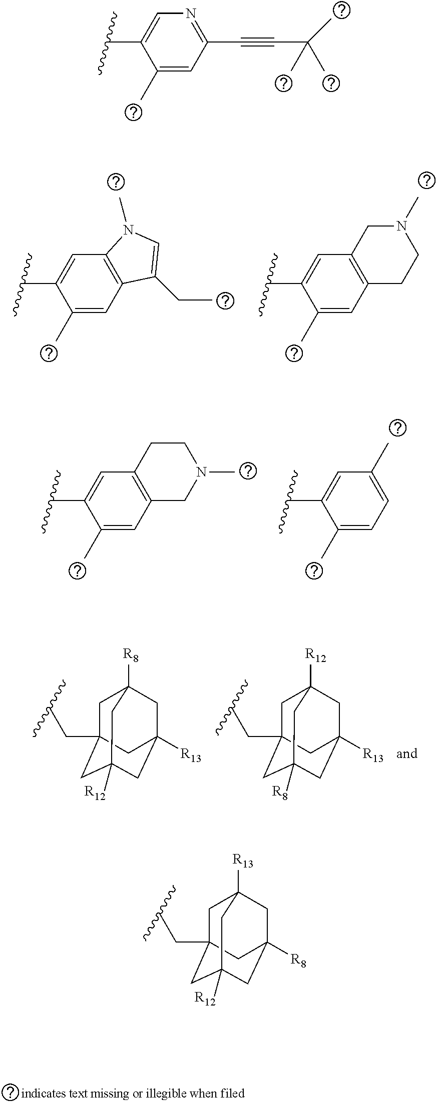

wherein Cy represents a C₃-C₈cycloalkyl,
R₈ represents a group selected from: hydrogen; linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}OR′_{c}; —O—X′₂—NR′ₐR′_{b}; —X′₂—NR′ₐR′_{b}: —NR′_{c}—X′₂—N₃ and:

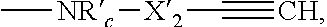

R₁₀ represents a group selected from hydrogen, fluorine, chlorine, bromine, —CF₃ and methyl,
R₁₁ represents a group selected from hydrogen, C₁-C₃alkylene-R₈, —O—C₁-C₆alkylene-R₈, —CO—NRₕRᵢ and —CH═CH—C₁-C₄alkylene-NRₕRᵢ, —CH═CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O; C₁-C₆alkylene-COOH; C₁-C₆alkylene-PO(OH)₂; C₁-C₆alkylene-NR′_{d}R′ₑ; C₁-C₆alkylene-N⁺R′_{d}R′ₑR′_{f}; C₁-C₆alkylene-O—C₁-C₆alkylene-OH; C₁-C₆alkylene-phenyl wherein the phenyl may be substituted by a hydroxyl or a C₁-C₆alkoxy group;
the group:

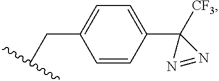 [SMILES: *Cc1ccc(C2(C(F)(F)F)N=N2)cc1]

or R′ₐ and R′_{b} form with the nitrogen atom carrying them a cycle B₃,
or R′ₐ, R′_{b} and R′_{c} form with the nitrogen atom carrying them a bridged C₃-C₈heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈heterocycloalkyl,
m=0, 1 or 2,
p=1, 2, 3 or 4,
B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl.

In some embodiments, for Formula (IA) or (IIA), G is selected from the group consisting of: —C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN, in which:
R_{G1} and R_{G2} at each occurrence are each independently selected from the group consisting of hydrogen, and C₁-C₆alkyl optionally substituted by 1 to 3 halogen atoms;
R_{G3} is C₁-C₆alkyl optionally substituted by 1 to 3 halogen atoms; or
R_{G1} and R_{G2}, together with the atom to which each is attached are combined to form a C₃-C₈heterocycloalkyl.

In some embodiments, for Formula (I), (II), (IA) or (IIA), R₇ represents a group selected from: linear or branched C₁-C₆alkyl group; (C₃-C₆) cycloalkylene-R₈; or:

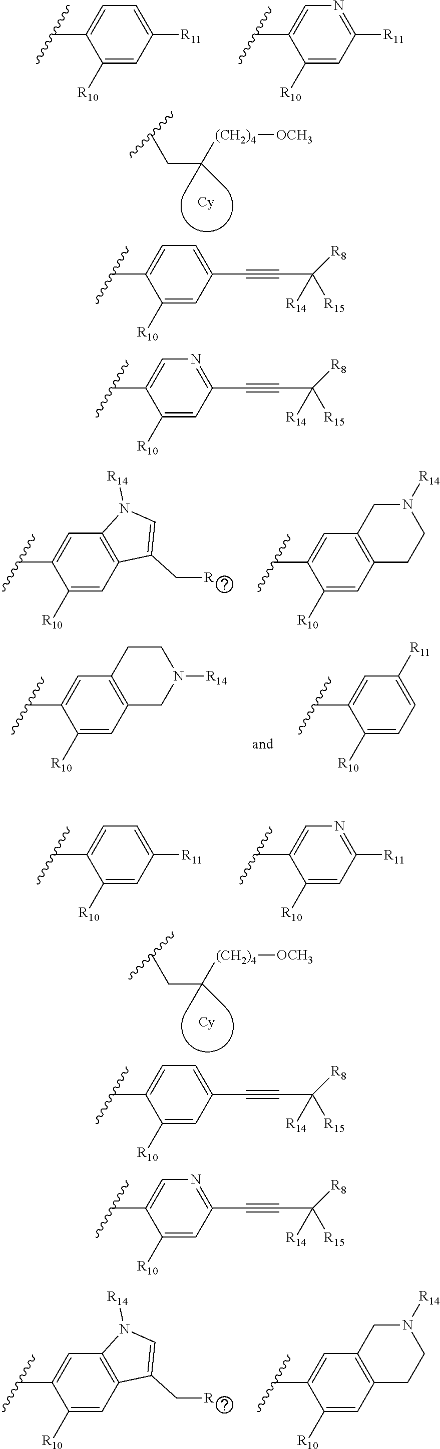

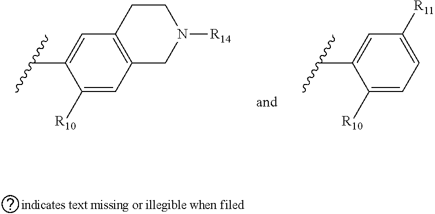

wherein Cy represents a C₃-C₈cycloalkyl.

In some embodiments, for Formula (I), (II), (IA) or (IIA), R₇ represents a group selected from:

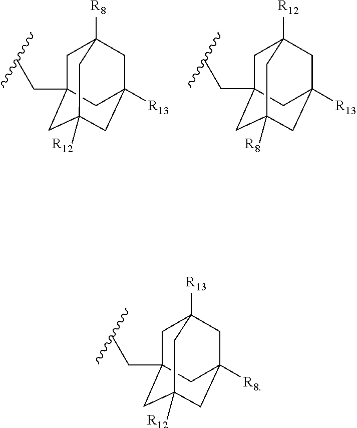

In some embodiments, the Bcl-xL inhibitor (D) comprises a compound of Formula (IB), (IC), (IIB) or (IIC):

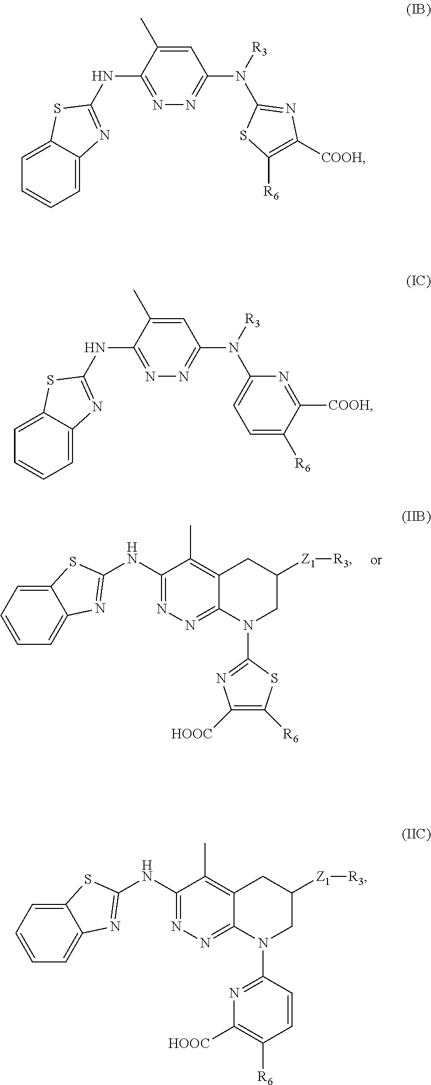

or an enantiomer, a diastereoisomer, and/or a pharmaceutically acceptable salt of any one of the foregoing, wherein:
for formula (IB) or (IC), R₃ represents a group selected from: hydrogen; linear or branched C₁-C₆alkyl; —X₁—NRₐR_{b}; —X₁—N⁺RₐR_{b}R_{c}; and —X₁—O—R_{c};
for formula (IIB) or (IIC), Z₁ represents a bond, and R₃ represents hydrogen; or Z₁ represents-O—, and R₃ represents —X₁—NRₐR_{b},
Rₐ and R_{b} independently of one another represent a group selected from: hydrogen; linear or branched C₁-C₆alkyl optionally substituted by one or two hydroxyl groups; and C₁-C₆alkylene-SO₂O,
R_{c} represents a hydrogen or a linear or branched C₁-C₆alkyl group
R₆ represents —X₂—O—R₇ or an heteroarylene-R₇ group optionally substituted by a linear or branched C₁-C₆alkyl group,
R₇ represents a group selected from:

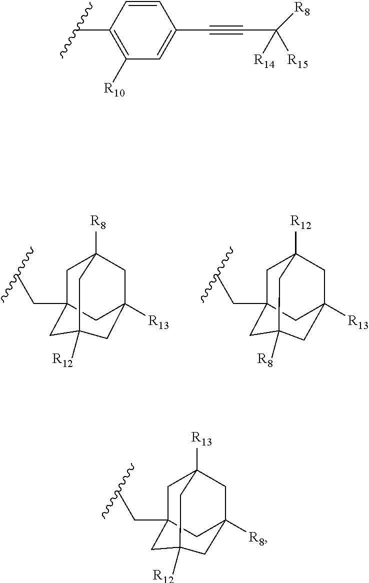

R₈ represents a group selected from: —NR′ₐR′_{b}; —O—X′₂—NR′ₐR′_{b}; and —X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; linear or branched C₁-C₆alkyl optionally substituted by one or two hydroxyl or C₁-C₆alkoxy groups; C₁-C₆alkylene-NR′ₐR′ₑ;
or R′ₐ and R′_{b} form with the nitrogen atom carrying them a cycle B₃,
R′_{d}, R′ₑ independently of one another, represents a hydrogen or a linear or branched C₁-C₆alkyl group,
B₃ represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from: fluorine, bromine, chlorine, linear or branched C₁-C₆alkyl, hydroxyl, and oxo.

In some embodiments, R₇ represents the following group:

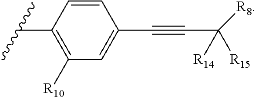

In some embodiments, R₇ represents a group selected from:

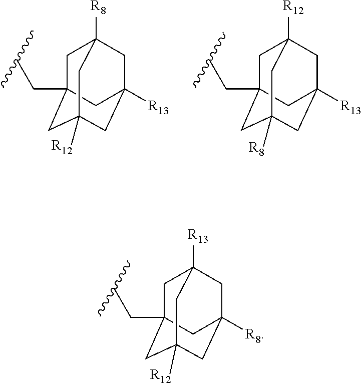

In some embodiments, for Formula (I), (IA), (IB), (IC), (II), (IIA), (IIB) or (IIC), R₈ represents a group selected from:

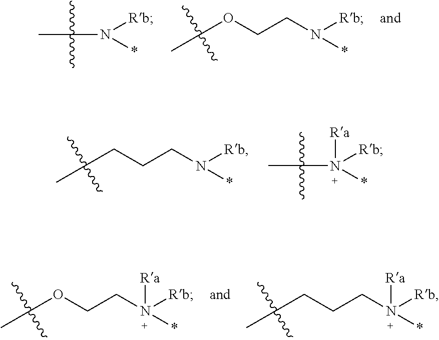

wherein [symbol] represents a bond to the linker.

In some embodiments, B3 represents a C3-C8heterocycloalkyl group selected from a pyrrolidinyl group, a piperidinyl group, a piperazinyl group, a morpholinyl group, an azepanyl group, and a 2,8-diazaspiro[4,5]decanyl group.

In some embodiments, D represents a Bcl-xL inhibitor attached to the linker L by a covalent bond, wherein the Bcl-xL inhibitor is selected from a compound in Table A1:

TABLE A1

| | |
|---|---|
| 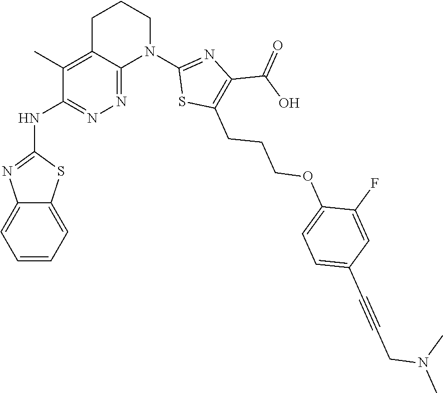 [SMILES: Cc1c(Nc2nc3ccccc3s2)nnc2c1CCCN2c1nc(C(=O)O)c(CCCOc2ccc(C#CCN(C)C)cc2F)s1] | P1 |
| 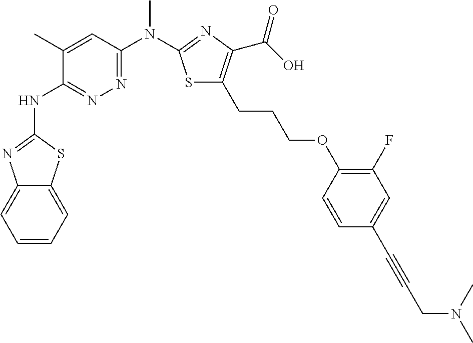 [SMILES: Cc1cc(N(C)c2nc(C(=O)O)c(CCCOc3ccc(C#CCN(C)C)cc3F)s2)nnc1Nc1nc2ccccc2s1] | P2 |
| 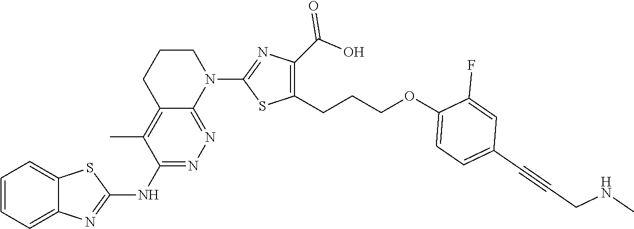 [SMILES: CNCC#Cc1ccc(OCCCc2sc(N3CCCc4c3nnc(Nc3nc5ccccc5s3)c4C)nc2C(=O)O)c(F)c1] | P3 |
| 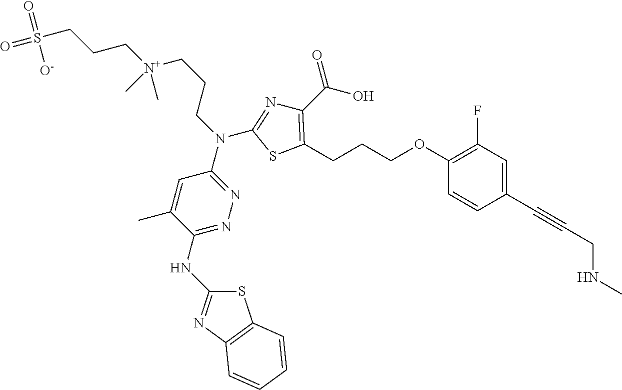 [SMILES: CNCC#Cc1ccc(OCCCc2sc(N(CCC[N+](C)(C)CCCS(=O)(=O)[O-])c3cc(C)c(Nc4nc5ccccc5s4)nn3)nc2C(=O)O)c(F)c1] | P4 |
| 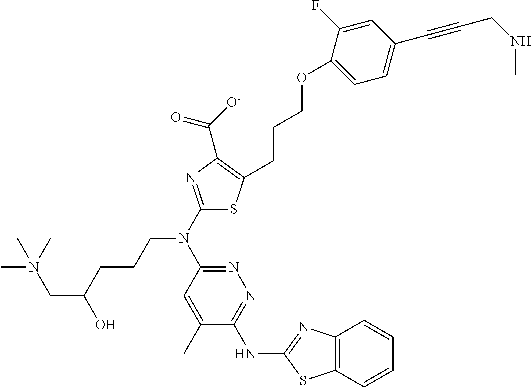 | P5 |
| 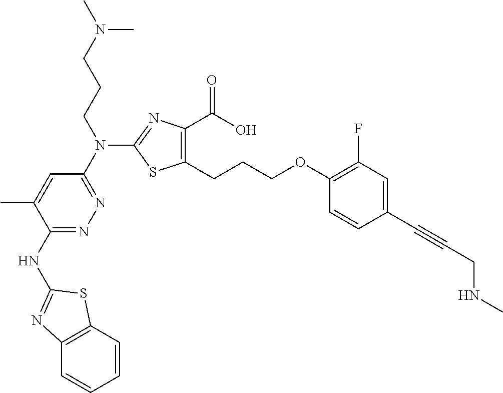 | P6 |
| 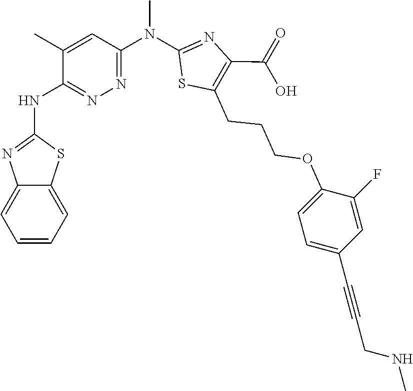 [SMILES: CNCC#Cc1ccc(OCCCc2sc(N(C)c3cc(C)c(Nc4nc5ccccc5s4)nn3)nc2C(=O)O)c(F)c1] | P7 |
| 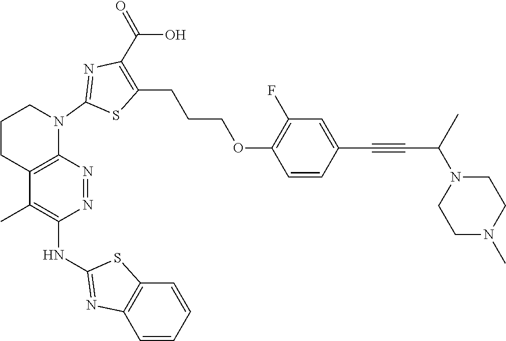 | P8 |
| 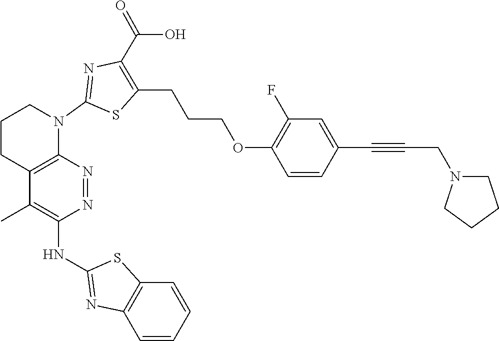 [SMILES: Cc1c(Nc2nc3ccccc3s2)nnc2c1CCCN2c1nc(C(=O)O)c(CCCOc2ccc(C#CCN3CCCC3)cc2F)s1] | P9 |
| 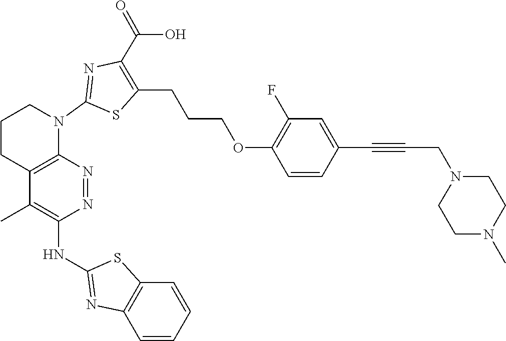 | P10 |
| 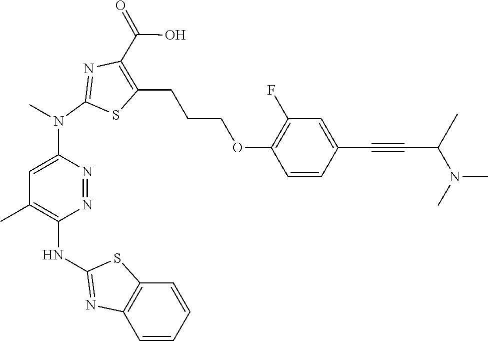 | P11 |
| 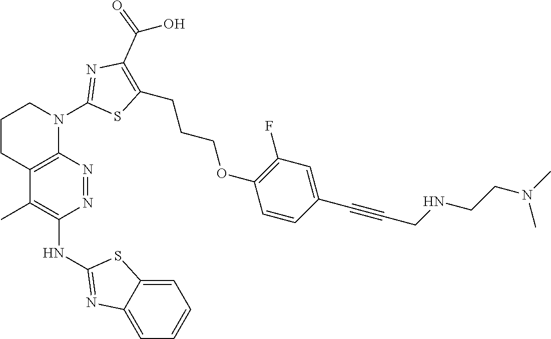 [SMILES: Cc1c(Nc2nc3ccccc3s2)nnc2c1CCCN2c1nc(C(=O)O)c(CCCOc2ccc(C#CCNCCN(C)C)cc2F)s1] | P12 |
| 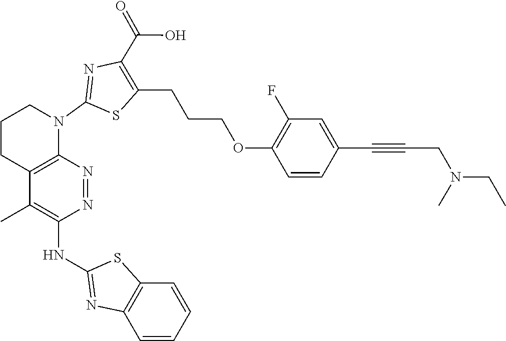 [SMILES: CCN(C)CC#Cc1ccc(OCCCc2sc(N3CCCc4c3nnc(Nc3nc5ccccc5s3)c4C)nc2C(=O)O)c(F)c1] | P13 |
| 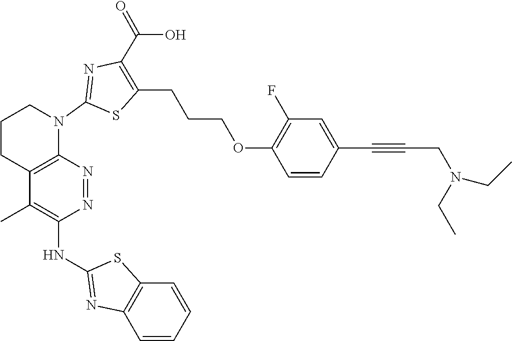 [SMILES: CCN(CC)CC#Cc1ccc(OCCCc2sc(N3CCCc4c3nnc(Nc3nc5ccccc5s3)c4C)nc2C(=O)O)c(F)c1] | P14 |
| 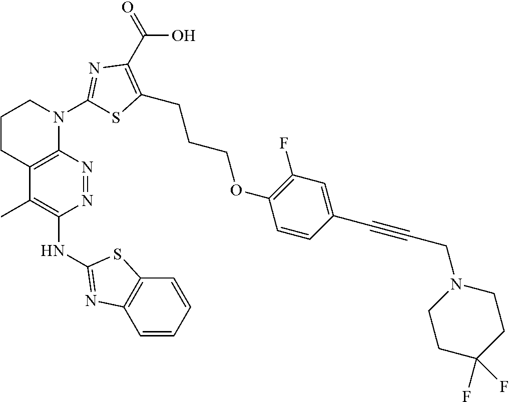 [SMILES: Cc1c(Nc2nc3ccccc3s2)nnc2c1CCCN2c1nc(C(=O)O)c(CCCOc2ccc(C#CCN3CCC(F)(F)CC3)cc2F)s1] | P15 |
| 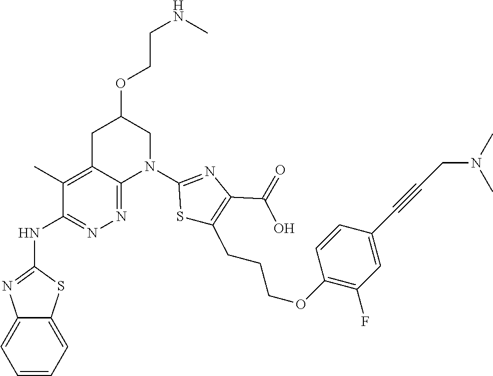 [SMILES: CNCCOC1Cc2c(nnc(Nc3nc4ccccc4s3)c2C)N(c2nc(C(=O)O)c(CCCOc3ccc(C#CCN(C)C)cc3F)s2)C1] | P16 |
| 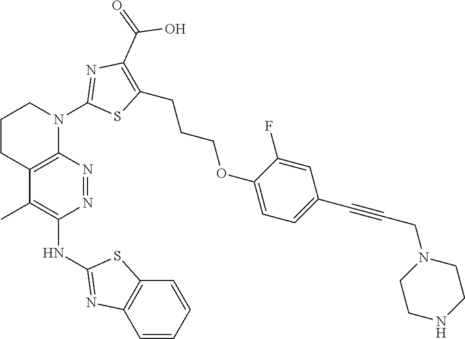 [SMILES: Cc1c(Nc2nc3ccccc3s2)nnc2c1CCCN2c1nc(C(=O)O)c(CCCOc2ccc(C#CCN3CCNCC3)cc2F)s1] | P17 |
| 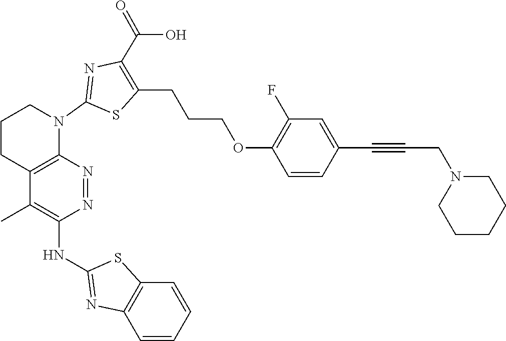 [SMILES: Cc1c(Nc2nc3ccccc3s2)nnc2c1CCCN2c1nc(C(=O)O)c(CCCOc2ccc(C#CCN3CCCCC3)cc2F)s1] | P18 |
| 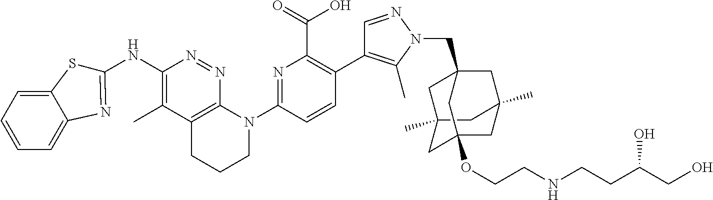 [SMILES: Cc1c(Nc2nc3ccccc3s2)nnc2c1CCCN2c1ccc(-c2cnn(C[C@]34C[C@]5(C)C[C@](C)(C3)C[C@@](OCCNCC[C@H](O)CO)(C5)C4)c2C)c(C(=O)O)n1] | P19 |
| 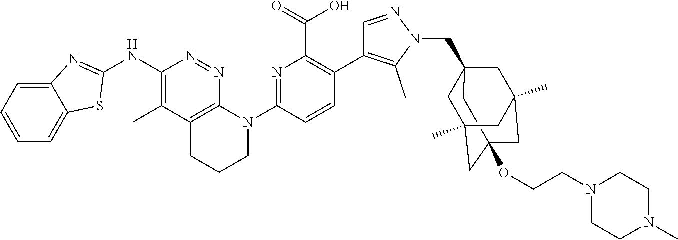 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCN(C)CC3)(C5)C4)c2C)c(C(=O)O)n1] | P20 |
| 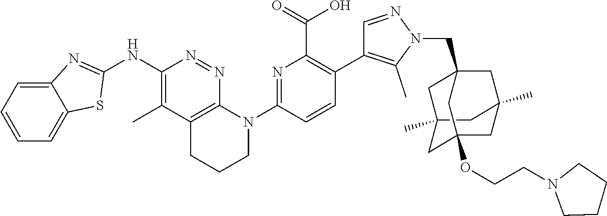 | P21 |
| 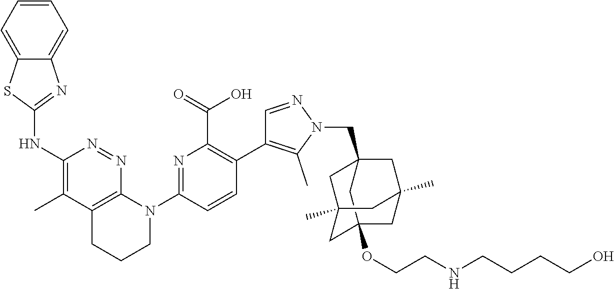 [SMILES: Cc1c(Nc2nc3ccccc3s2)nnc2c1CCCN2c1ccc(-c2cnn(C[C@]34C[C@]5(C)C[C@](C)(C3)C[C@@](OCCNCCCCO)(C5)C4)c2C)c(C(=O)O)n1] | P22 |
| 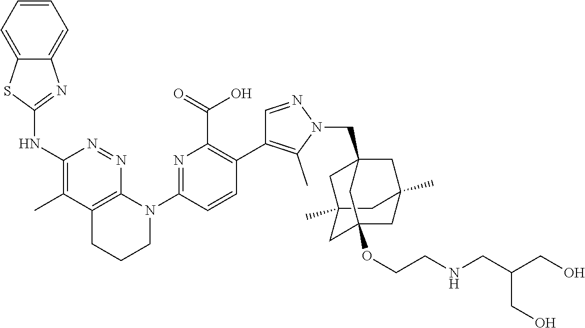 [SMILES: Cc1c(Nc2nc3ccccc3s2)nnc2c1CCCN2c1ccc(-c2cnn(C[C@]34C[C@]5(C)C[C@](C)(C3)C[C@@](OCCNCC(CO)CO)(C5)C4)c2C)c(C(=O)O)n1] | P23 |
| 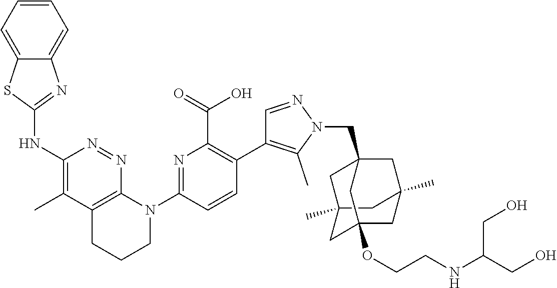 [SMILES: Cc1c(Nc2nc3ccccc3s2)nnc2c1CCCN2c1ccc(-c2cnn(C[C@]34C[C@]5(C)C[C@](C)(C3)C[C@@](OCCNC(CO)CO)(C5)C4)c2C)c(C(=O)O)n1] | P24 |
| 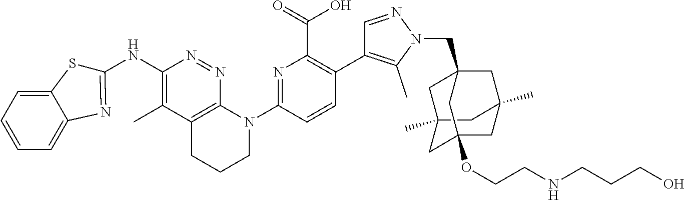 [SMILES: Cc1c(Nc2nc3ccccc3s2)nnc2c1CCCN2c1ccc(-c2cnn(C[C@]34C[C@]5(C)C[C@](C)(C3)C[C@@](OCCNCCCO)(C5)C4)c2C)c(C(=O)O)n1] | P25 |
| 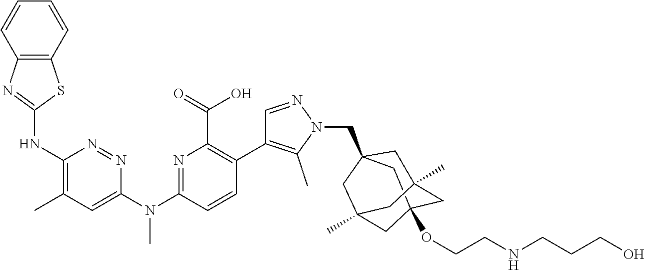 [SMILES: Cc1cc(N(C)c2ccc(-c3cnn(C[C@]45C[C@]6(C)C[C@](C)(C4)C[C@@](OCCNCCCO)(C6)C5)c3C)c(C(=O)O)n2)nnc1Nc1nc2ccccc2s1] | P26 |
| 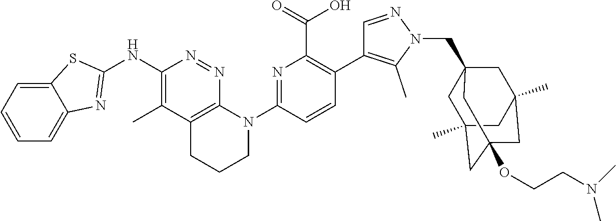 [SMILES: Cc1c(Nc2nc3ccccc3s2)nnc2c1CCCN2c1ccc(-c2cnn(C[C@]34C[C@]5(C)C[C@](C)(C3)C[C@@](OCCN(C)C)(C5)C4)c2C)c(C(=O)O)n1] | P27 |
| 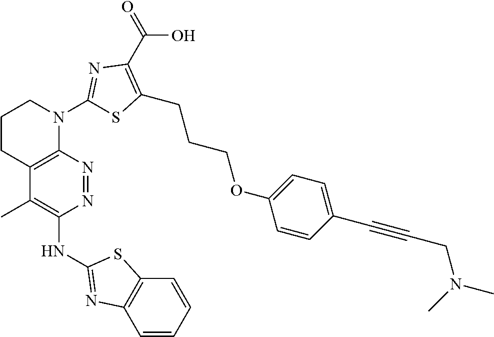 [SMILES: Cc1c(Nc2nc3ccccc3s2)nnc2c1CCCN2c1nc(C(=O)O)c(CCCOc2ccc(C#CCN(C)C)cc2)s1] | P28 |
| 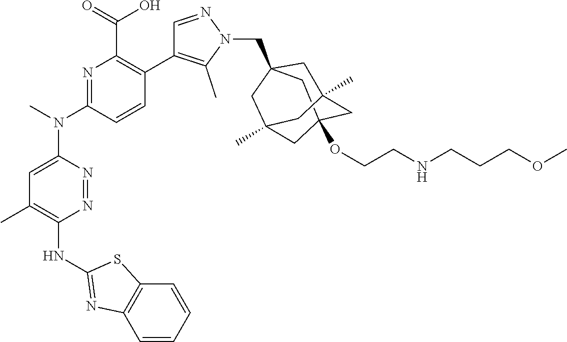 | P29 |
| 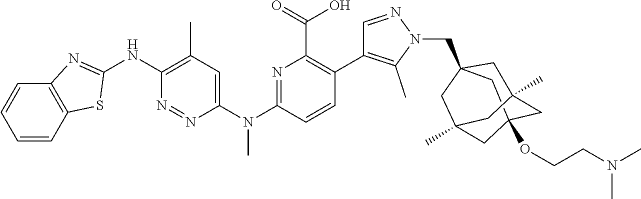 [SMILES: Cc1cc(N(C)c2ccc(-c3cnn(C[C@]45C[C@]6(C)C[C@](C)(C4)C[C@@](OCCN(C)C)(C6)C5)c3C)c(C(=O)O)n2)nnc1Nc1nc2ccccc2s1] | P30 |
| 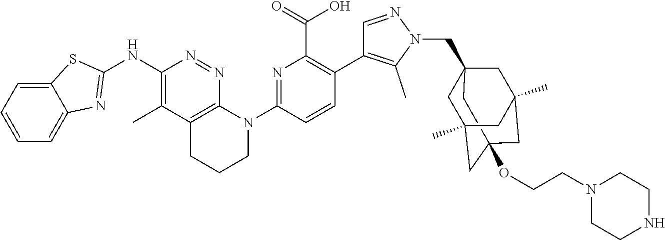 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCNCC3)(C5)C4)c2C)c(C(=O)O)n1] | P31 |
| 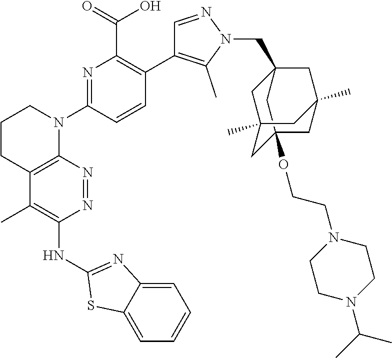 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCN(C(C)C)CC3)(C5)C4)c2C)c(C(=O)O)n1] | P32 |
| 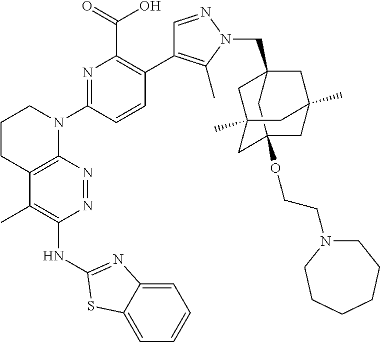 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCCCCC3)(C5)C4)c2C)c(C(=O)O)n1] | P33 |
| 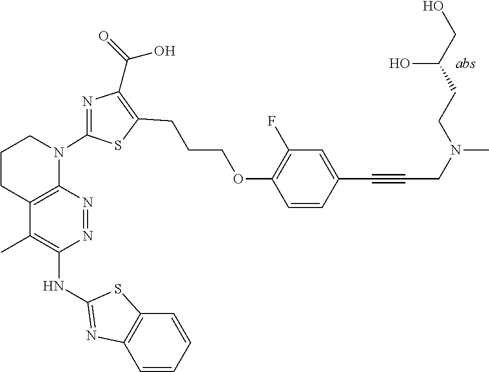 | P34 |
| 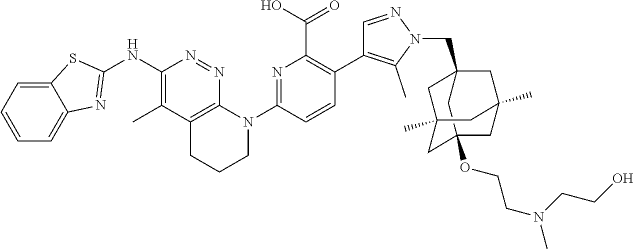 [SMILES: Cc1c(Nc2nc3ccccc3s2)nnc2c1CCCN2c1ccc(-c2cnn(C[C@]34C[C@]5(C)C[C@](C)(C3)C[C@@](OCCN(C)CCO)(C5)C4)c2C)c(C(=O)O)n1] | P35 |
| 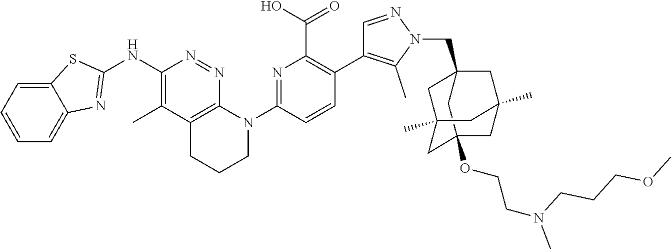 [SMILES: COCCCN(C)CCO[C@]12C[C@@]3(C)C[C@](C)(C[C@](Cn4ncc(-c5ccc(N6CCCc7c6nnc(Nc6nc8ccccc8s6)c7C)nc5C(=O)O)c4C)(C3)C1)C2] | P36 |
| 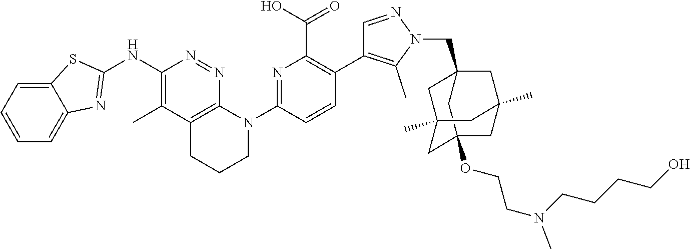 [SMILES: Cc1c(Nc2nc3ccccc3s2)nnc2c1CCCN2c1ccc(-c2cnn(C[C@]34C[C@]5(C)C[C@](C)(C3)C[C@@](OCCN(C)CCCCO)(C5)C4)c2C)c(C(=O)O)n1] | P37 |
| 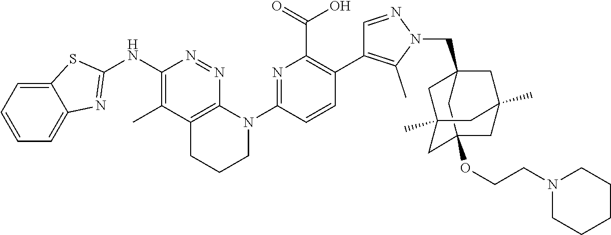 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCCCC3)(C5)C4)c2C)c(C(=O)O)n1] | P38 |
| 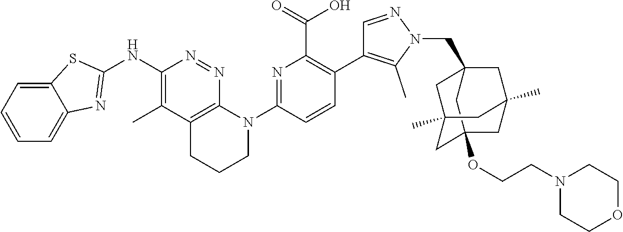 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCOCC3)(C5)C4)c2C)c(C(=O)O)n1] | P39 |
| 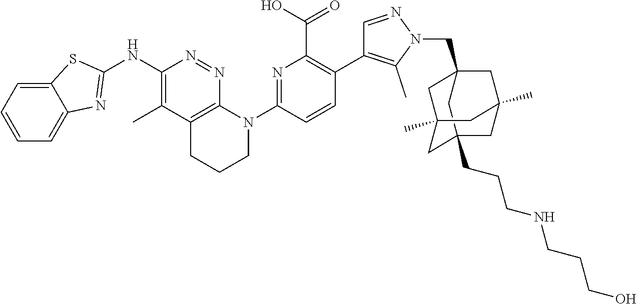 [SMILES: Cc1c(Nc2nc3ccccc3s2)nnc2c1CCCN2c1ccc(-c2cnn(C[C@]34C[C@@]5(C)C[C@](C)(C[C@](CCCNCCCO)(C5)C3)C4)c2C)c(C(=O)O)n1] | P40 |
| 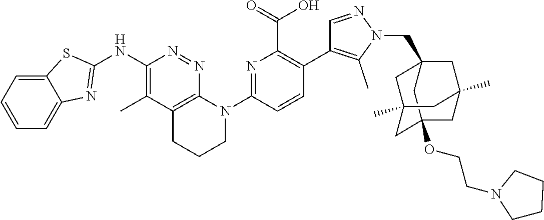 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCCC3)(C5)C4)c2C)c(C(=O)O)n1] | P41 |
| 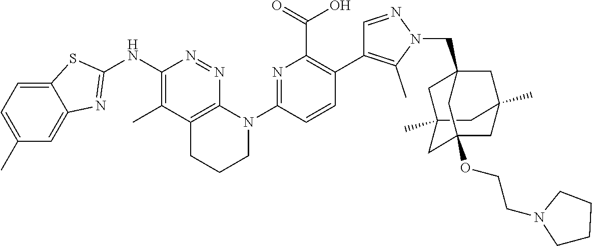 [SMILES: Cc1ccc2sc(Nc3nnc4c(c3C)CCCN4c3ccc(-c4cnn(C[C@]56C[C@]7(C)C[C@](C)(C5)C[C@@](OCCN5CCCC5)(C7)C6)c4C)c(C(=O)O)n3)nc2c1] | P42 |
| 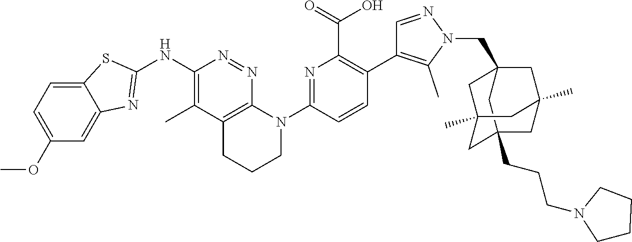 [SMILES: COc1ccc2sc(Nc3nnc4c(c3C)CCCN4c3ccc(-c4cnn(C[C@]56C[C@@]7(C)C[C@](C)(C[C@](CCCN8CCCC8)(C7)C5)C6)c4C)c(C(=O)O)n3)nc2c1] | P43 |
| 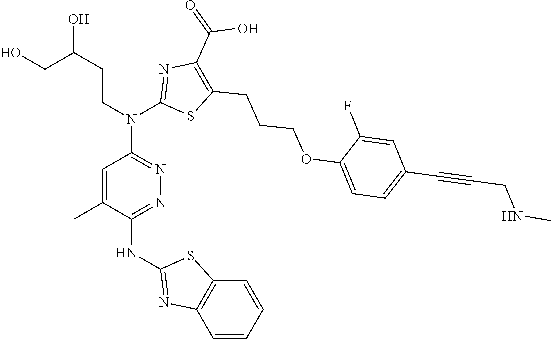 [SMILES: CNCC#Cc1ccc(OCCCc2sc(N(CCC(O)CO)c3cc(C)c(Nc4nc5ccccc5s4)nn3)nc2C(=O)O)c(F)c1] | P44 |
| 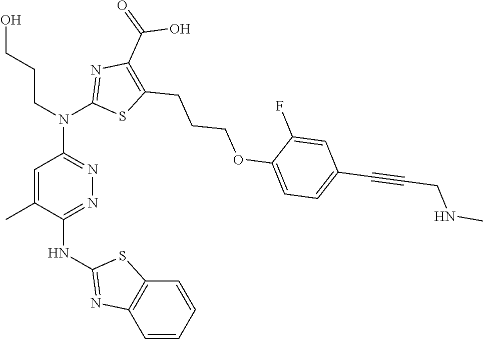 | P45 |
| 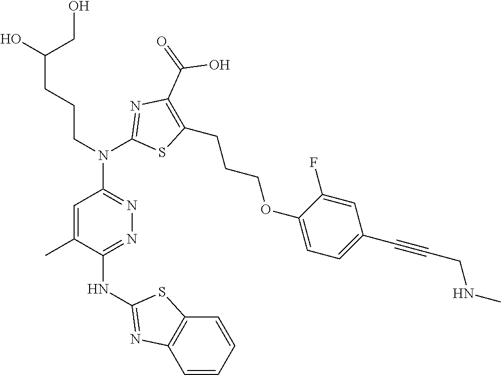 [SMILES: CNCC#Cc1ccc(OCCCc2sc(N(CCCC(O)CO)c3cc(C)c(Nc4nc5ccccc5s4)nn3)nc2C(=O)O)c(F)c1] | P46 |
| 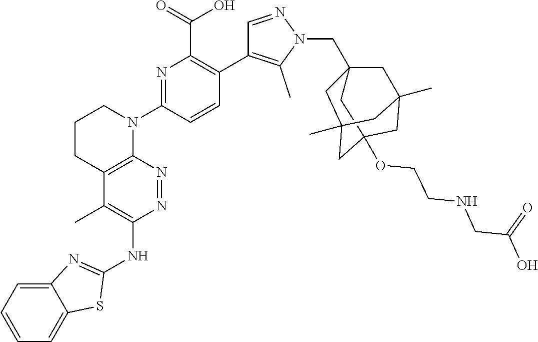 [SMILES: Cc1c(Nc2nc3ccccc3s2)nnc2c1CCCN2c1ccc(-c2cnn(CC34CC5(C)CC(C)(C3)CC(OCCNCC(=O)O)(C5)C4)c2C)c(C(=O)O)n1] | P47 |
| 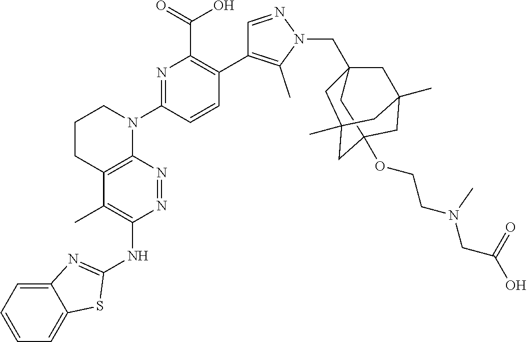 [SMILES: Cc1c(Nc2nc3ccccc3s2)nnc2c1CCCN2c1ccc(-c2cnn(CC34CC5(C)CC(C)(C3)CC(OCCN(C)CC(=O)O)(C5)C4)c2C)c(C(=O)O)n1] | P48 |
| 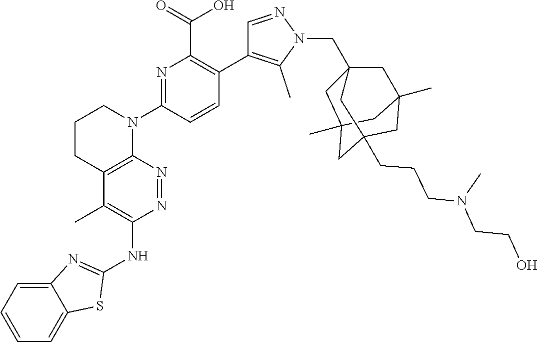 [SMILES: Cc1c(Nc2nc3ccccc3s2)nnc2c1CCCN2c1ccc(-c2cnn(CC34CC5(C)CC(C)(CC(CCCN(C)CCO)(C5)C3)C4)c2C)c(C(=O)O)n1] | P49 |
| 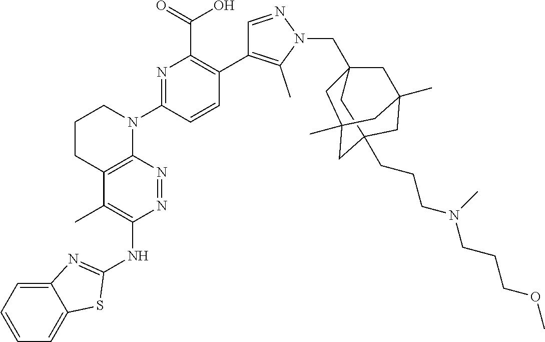 [SMILES: COCCCN(C)CCCC12CC3(C)CC(C)(C1)CC(Cn1ncc(-c4ccc(N5CCCc6c5nnc(Nc5nc7ccccc7s5)c6C)nc4C(=O)O)c1C)(C3)C2] | P50 |
| 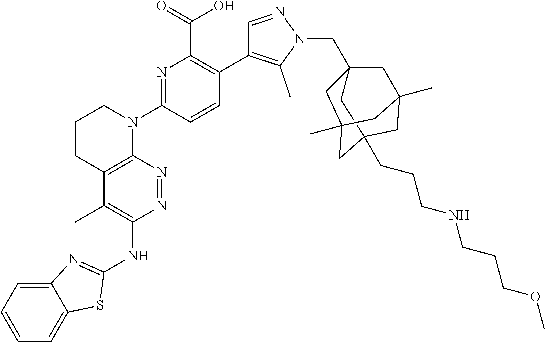 [SMILES: COCCCNCCCC12CC3(C)CC(C)(C1)CC(Cn1ncc(-c4ccc(N5CCCc6c5nnc(Nc5nc7ccccc7s5)c6C)nc4C(=O)O)c1C)(C3)C2] | P51 |
| 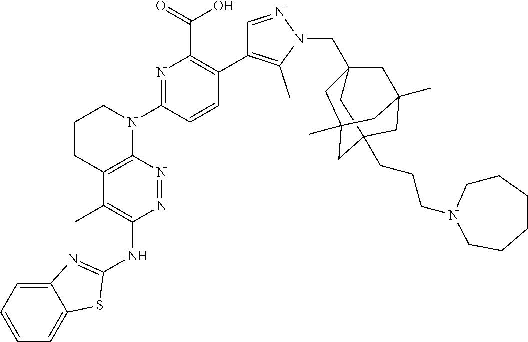 [SMILES: Cc1c(Nc2nc3ccccc3s2)nnc2c1CCCN2c1ccc(-c2cnn(CC34CC5(C)CC(C)(CC(CCCN6CCCCCC6)(C5)C3)C4)c2C)c(C(=O)O)n1] | P52 |
| 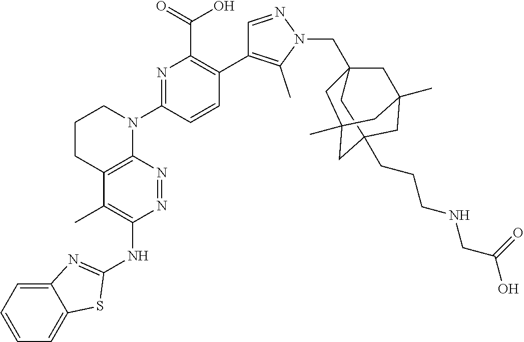 [SMILES: Cc1c(Nc2nc3ccccc3s2)nnc2c1CCCN2c1ccc(-c2cnn(CC34CC5(C)CC(C)(CC(CCCNCC(=O)O)(C5)C3)C4)c2C)c(C(=O)O)n1] | P53 |
| 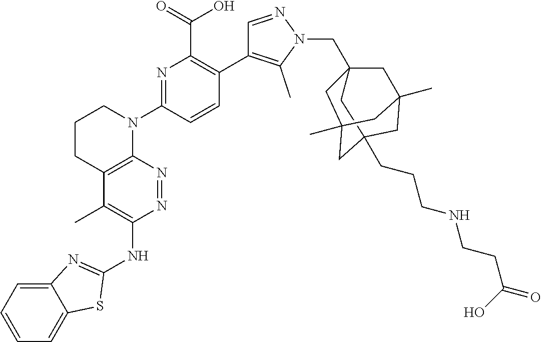 [SMILES: Cc1c(Nc2nc3ccccc3s2)nnc2c1CCCN2c1ccc(-c2cnn(CC34CC5(C)CC(C)(CC(CCCNCCC(=O)O)(C5)C3)C4)c2C)c(C(=O)O)n1] | P54 |
| 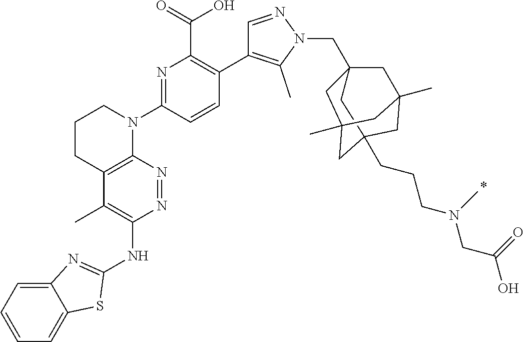 [SMILES: Cc1c(Nc2nc3ccccc3s2)nnc2c1CCCN2c1ccc(-c2cnn(CC34CC5(C)CC(C)(CC(CCCN(C)CC(=O)O)(C5)C3)C4)c2C)c(C(=O)O)n1] | P55 |
| 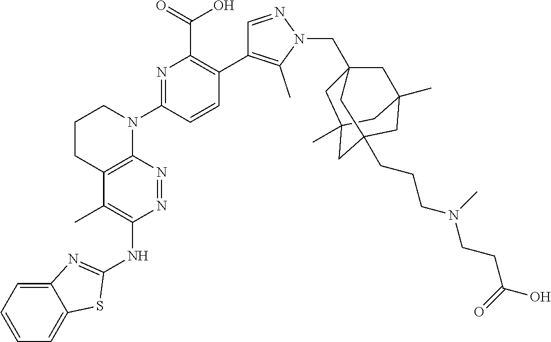 [SMILES: Cc1c(Nc2nc3ccccc3s2)nnc2c1CCCN2c1ccc(-c2cnn(CC34CC5(C)CC(C)(CC(CCCN(C)CCC(=O)O)(C5)C3)C4)c2C)c(C(=O)O)n1] | P56 |
| 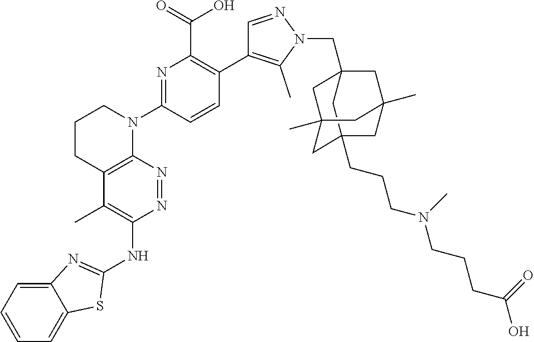 [SMILES: Cc1c(Nc2nc3ccccc3s2)nnc2c1CCCN2c1ccc(-c2cnn(CC34CC5(C)CC(C)(CC(CCCN(C)CCCC(=O)O)(C5)C3)C4)c2C)c(C(=O)O)n1] | P57 |
| 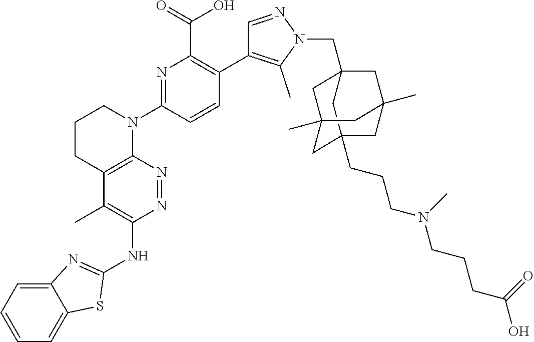 [SMILES: Cc1c(Nc2nc3ccccc3s2)nnc2c1CCCN2c1ccc(-c2cnn(CC34CC5(C)CC(C)(CC(CCCN(C)CCCC(=O)O)(C5)C3)C4)c2C)c(C(=O)O)n1] | P58 |
| 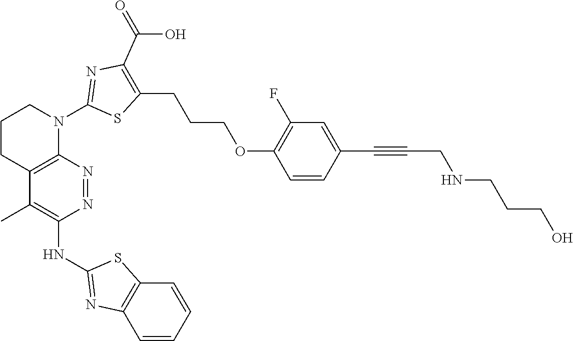 [SMILES: Cc1c(Nc2nc3ccccc3s2)nnc2c1CCCN2c1nc(C(=O)O)c(CCCOc2ccc(C#CCNCCCO)cc2F)s1] | P59 |
| 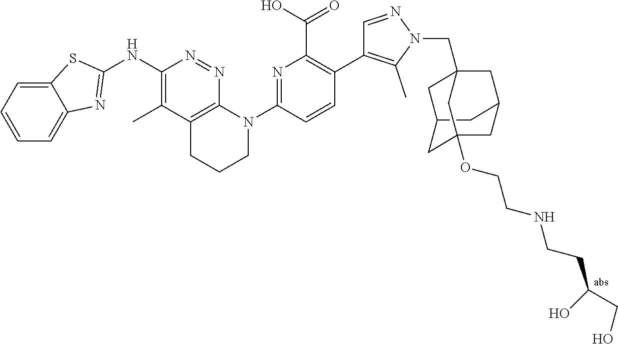 | P60 |
| 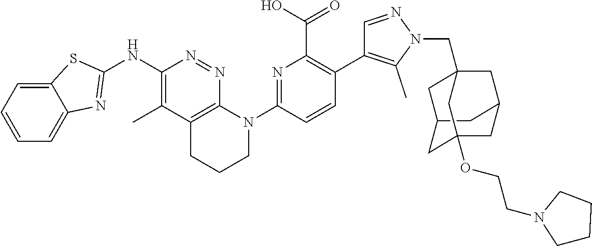 [SMILES: Cc1c(Nc2nc3ccccc3s2)nnc2c1CCCN2c1ccc(-c2cnn(CC34CC5CC(C3)CC(OCCN3CCCC3)(C5)C4)c2C)c(C(=O)O)n1] | P61 |
| 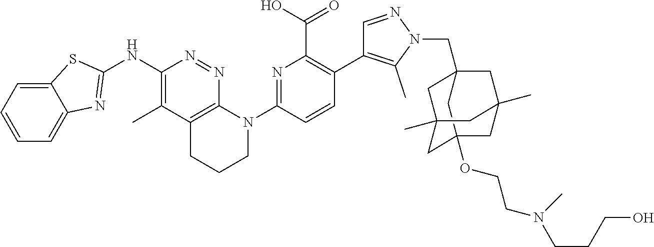 [SMILES: Cc1c(Nc2nc3ccccc3s2)nnc2c1CCCN2c1ccc(-c2cnn(CC34CC5(C)CC(C)(C3)CC(OCCN(C)CCCO)(C5)C4)c2C)c(C(=O)O)n1] | P62 |
| 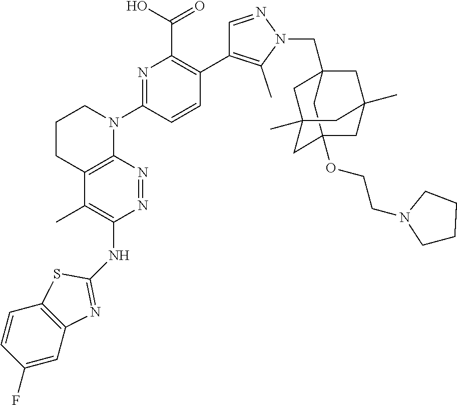 [SMILES: Cc1c(Nc2nc3cc(F)ccc3s2)nnc2c1CCCN2c1ccc(-c2cnn(CC34CC5(C)CC(C)(C3)CC(OCCN3CCCC3)(C5)C4)c2C)c(C(=O)O)n1] | P63 |
| 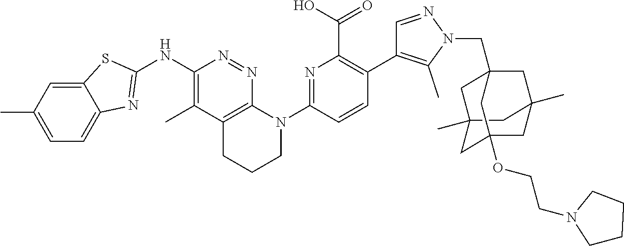 [SMILES: Cc1ccc2nc(Nc3nnc4c(c3C)CCCN4c3ccc(-c4cnn(CC56CC7(C)CC(C)(C5)CC(OCCN5CCCC5)(C7)C6)c4C)c(C(=O)O)n3)sc2c1] | P64 |
| 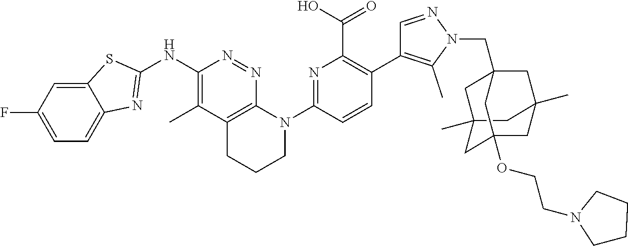 [SMILES: Cc1c(Nc2nc3ccc(F)cc3s2)nnc2c1CCCN2c1ccc(-c2cnn(CC34CC5(C)CC(C)(C3)CC(OCCN3CCCC3)(C5)C4)c2C)c(C(=O)O)n1] | P65 |
| 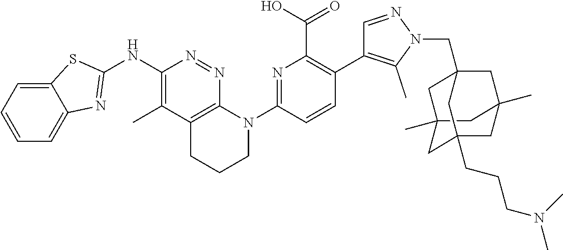 [SMILES: Cc1c(Nc2nc3ccccc3s2)nnc2c1CCCN2c1ccc(-c2cnn(CC34CC5(C)CC(C)(CC(CCCN(C)C)(C5)C3)C4)c2C)c(C(=O)O)n1] | P66 |
| 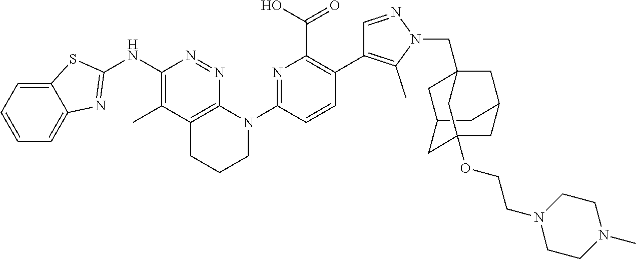 [SMILES: Cc1c(Nc2nc3ccccc3s2)nnc2c1CCCN2c1ccc(-c2cnn(CC34CC5CC(C3)CC(OCCN3CCN(C)CC3)(C5)C4)c2C)c(C(=O)O)n1] | P67 |
| 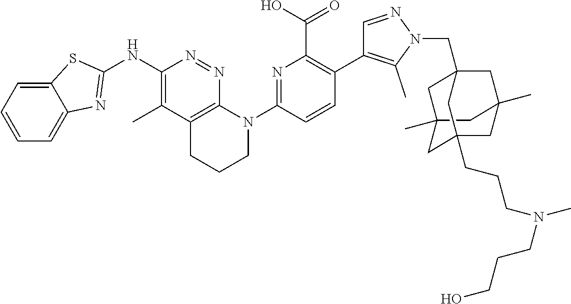 [SMILES: Cc1c(Nc2nc3ccccc3s2)nnc2c1CCCN2c1ccc(-c2cnn(CC34CC5(C)CC(C)(CC(CCCN(C)CCCO)(C5)C3)C4)c2C)c(C(=O)O)n1] | P68 |
| 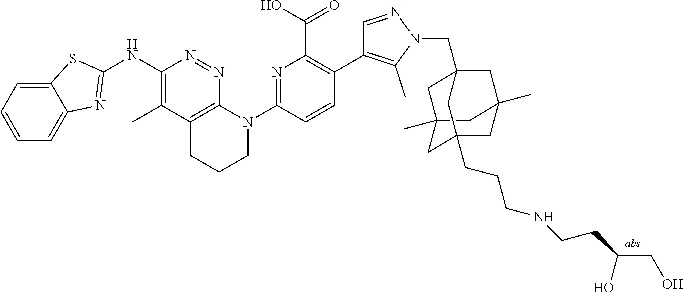 [SMILES: Cc1c(Nc2nc3ccccc3s2)nnc2c1CCCN2c1ccc(-c2cnn(CC34CC5(C)CC(C)(CC(CCCNCC[C@H](O)CO)(C5)C3)C4)c2C)c(C(=O)O)n1] | P69 |
| 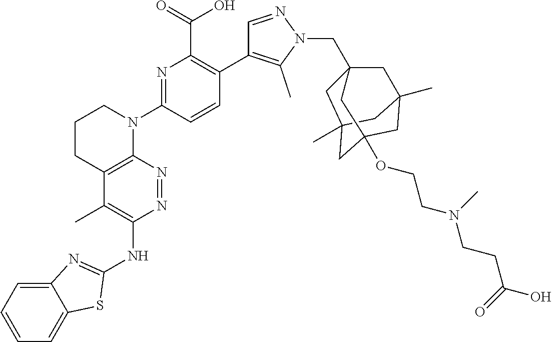 [SMILES: Cc1c(Nc2nc3ccccc3s2)nnc2c1CCCN2c1ccc(-c2cnn(CC34CC5(C)CC(C)(C3)CC(OCCN(C)CCC(=O)O)(C5)C4)c2C)c(C(=O)O)n1] | P70 |
| 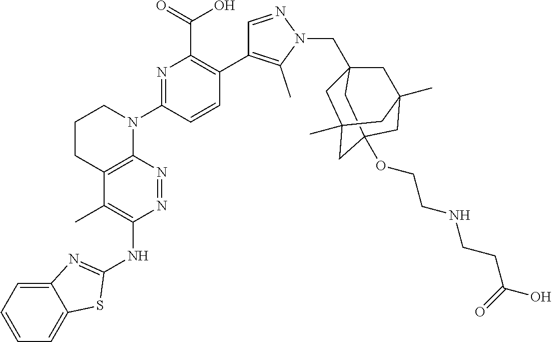 [SMILES: Cc1c(Nc2nc3ccccc3s2)nnc2c1CCCN2c1ccc(-c2cnn(CC34CC5(C)CC(C)(C3)CC(OCCNCCC(=O)O)(C5)C4)c2C)c(C(=O)O)n1] | P71 |
| 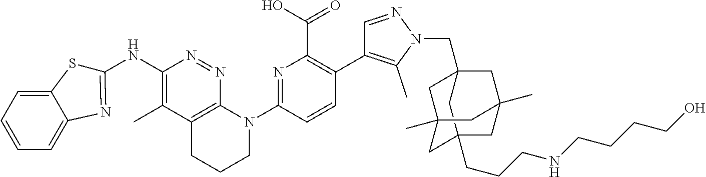 [SMILES: Cc1c(Nc2nc3ccccc3s2)nnc2c1CCCN2c1ccc(-c2cnn(CC34CC5(C)CC(C)(CC(CCCNCCCCO)(C5)C3)C4)c2C)c(C(=O)O)n1] | P72 |
| 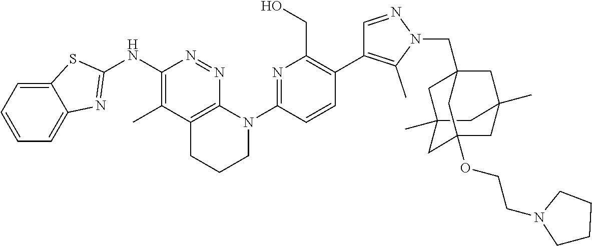 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(CO)n1] | P73 |
| 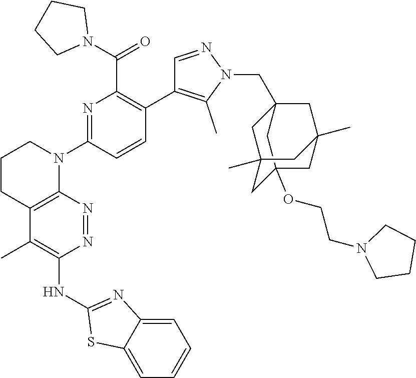 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)N2CCCC2)n1] | P74 |
| 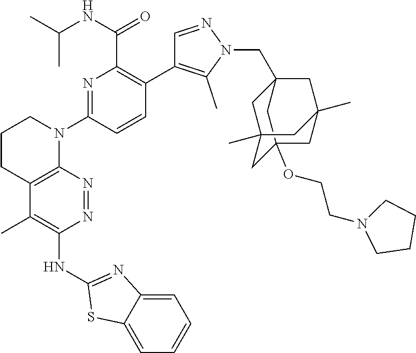 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)NC(C)C)n1] | P75 |
| 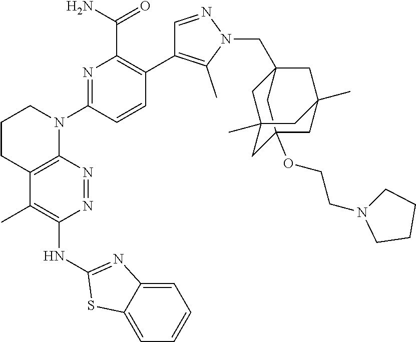 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(N)=O)n1] | P76 |

or an enantiomer, a diastereoisomer, and/or a pharmaceutically acceptable salt of any one of the foregoing.

In some embodiments, D comprises a formula selected from any one of the formulae in Table A2, or an enantiomer, a diastereoisomer, and/or a pharmaceutically acceptable salt of any one of the foregoing.

TABLE A2

| | |
|---|---|
| 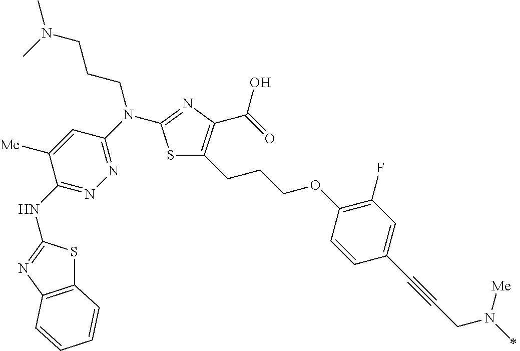 | P1a |
| 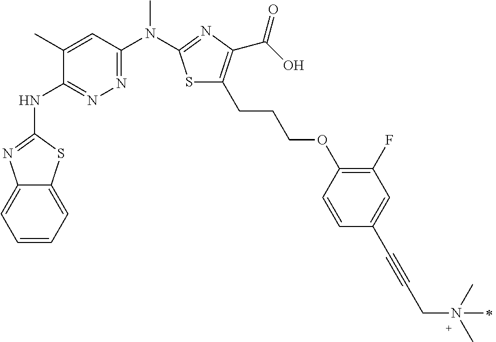 | P2a |
| 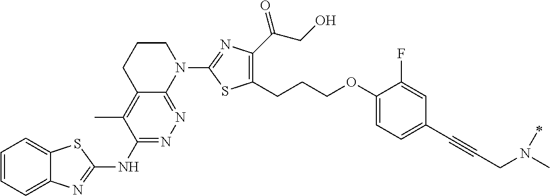 | P3a |
| 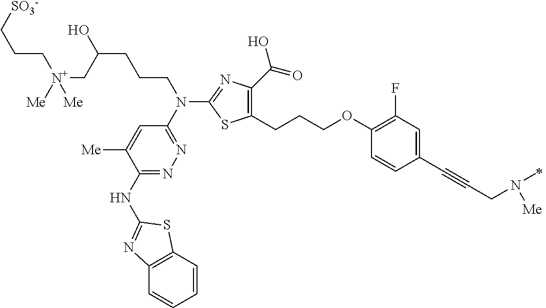 | P4a |
| 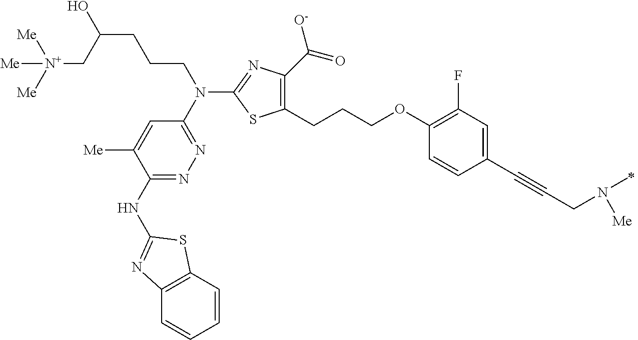 | P5a |
| 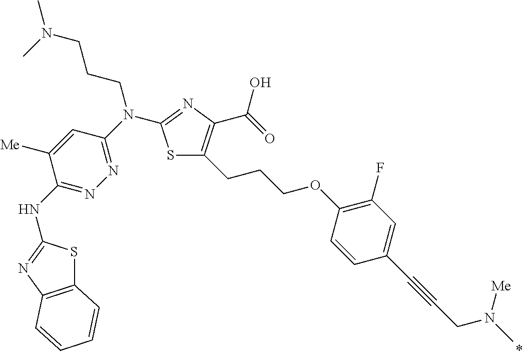 [SMILES: Cc1cc(N(CCCN(C)C)c2nc(C(=O)O)c(CCCOc3ccc(C#CCN(C)C)cc3F)s2)nnc1Nc1nc2ccccc2s1] | P6a |
| 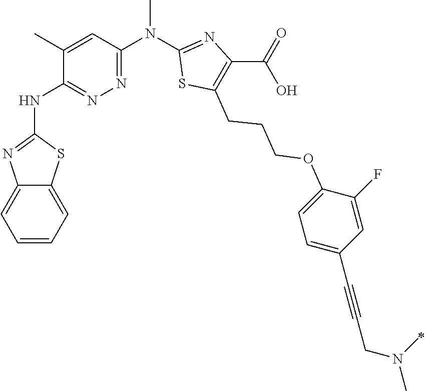 | P7a |
| 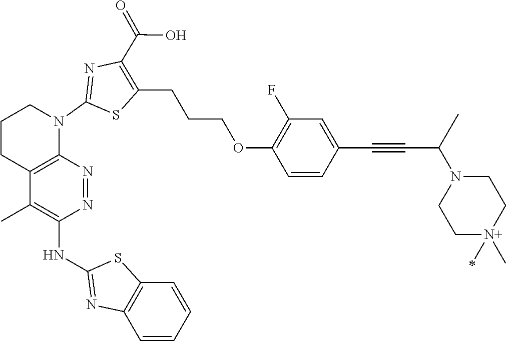 | P8a |
| 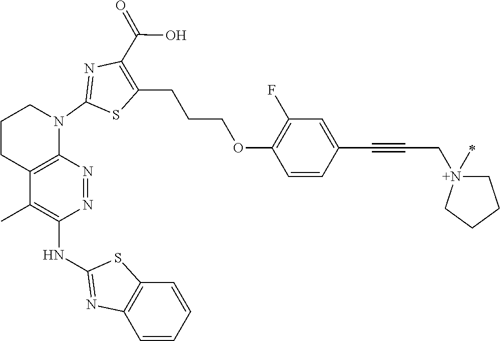 | P9a |
| 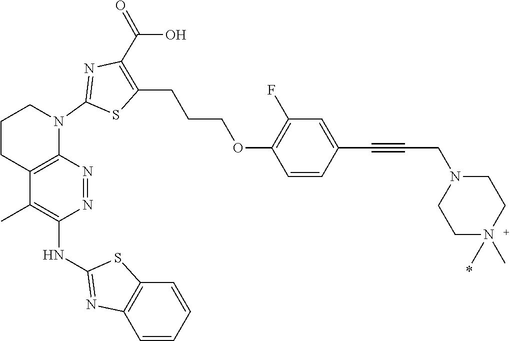 | P10a |
| 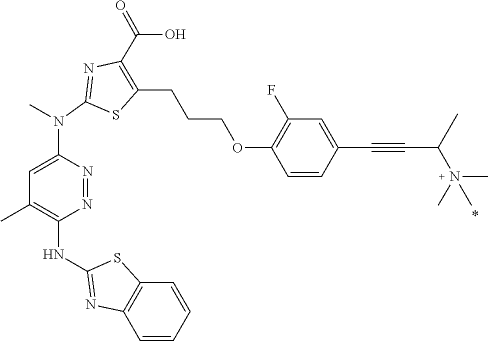 | P11a |
| 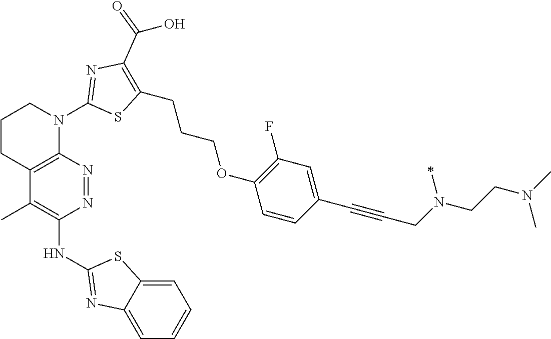 | P12a |
| 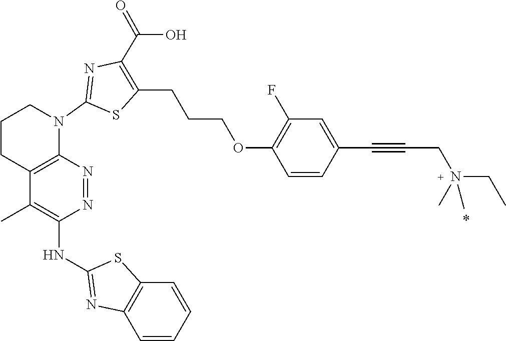 | P13a |
| 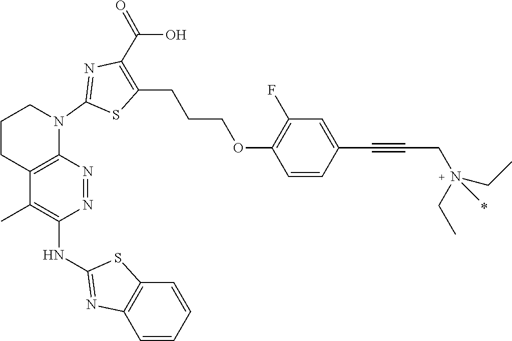 | P14a |
| 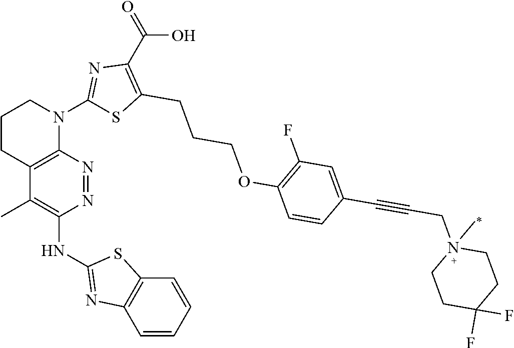 | P15a |
| 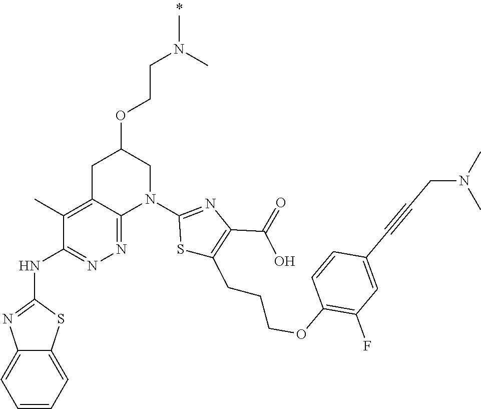 [SMILES: Cc1c(Nc2nc3ccccc3s2)nnc2c1CC(OCCN(C)C)CN2c1nc(C(=O)O)c(CCCOc2ccc(C#CCN(C)C)cc2F)s1] | P16a |
| 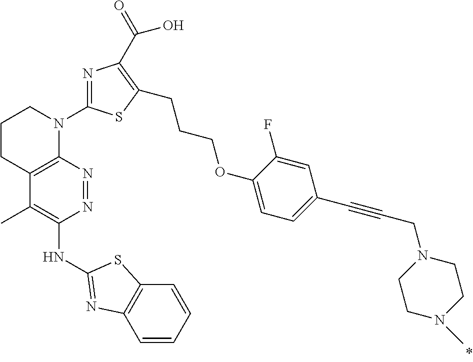 [SMILES: Cc1c(Nc2nc3ccccc3s2)nnc2c1CCCN2c1nc(C(=O)O)c(CCCOc2ccc(C#CCN3CCN(C)CC3)cc2F)s1] | P17a |
| 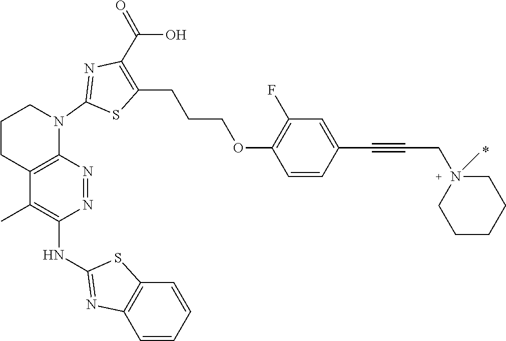 | P18a |
| 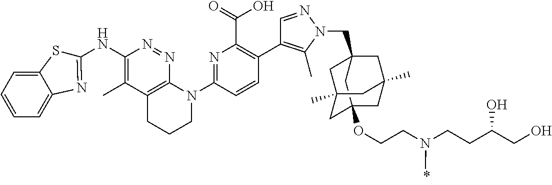 [SMILES: Cc1c(Nc2nc3ccccc3s2)nnc2c1CCCN2c1ccc(-c2cnn(C[C@]34C[C@]5(C)C[C@](C)(C3)C[C@@](OCCN(C)CC[C@H](O)CO)(C5)C4)c2C)c(C(=O)O)n1] | P19a |
| 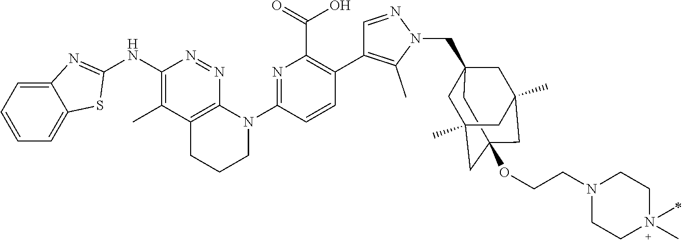 | P20a |
| 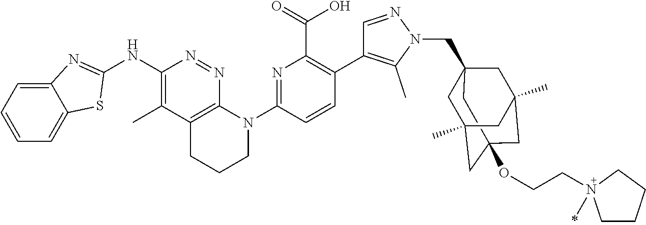 | P21a |
| 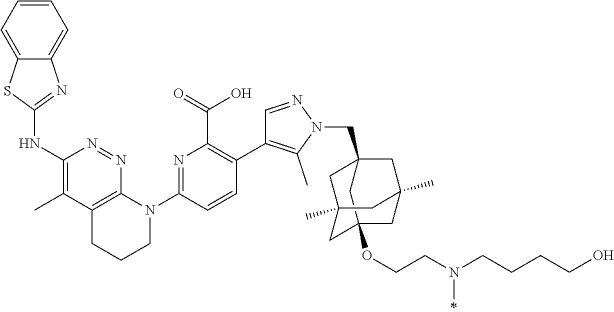 | P22a |
| 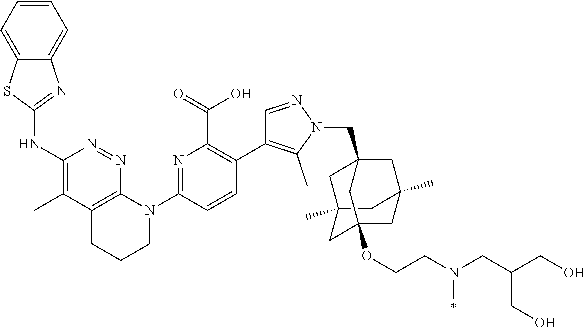 | P23a |
| 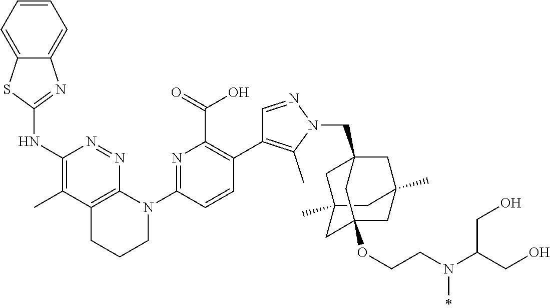 | P24a |
| 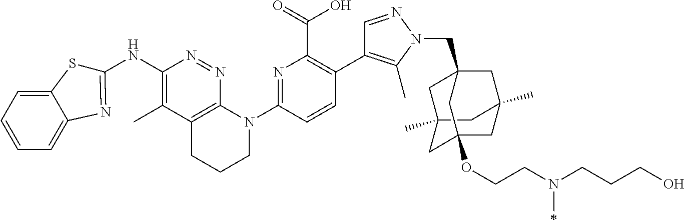 | P25a |
| 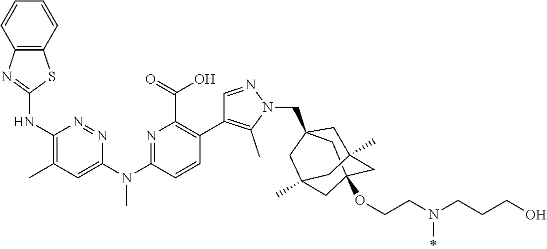 | P26a |
| 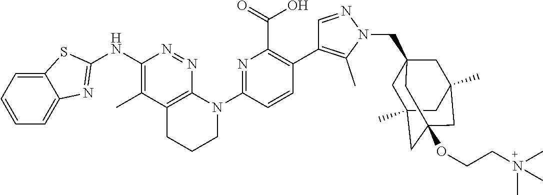 | P27a |
| 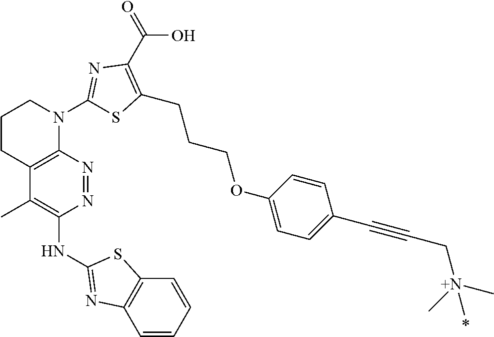 | P28a |
| 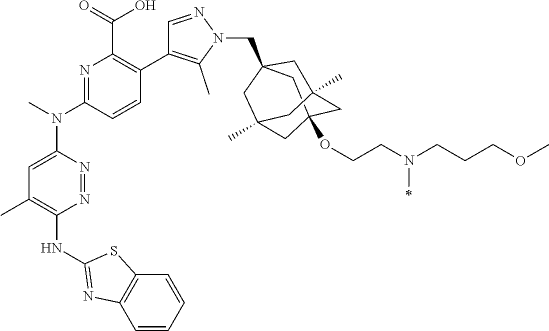 | P29a |
| 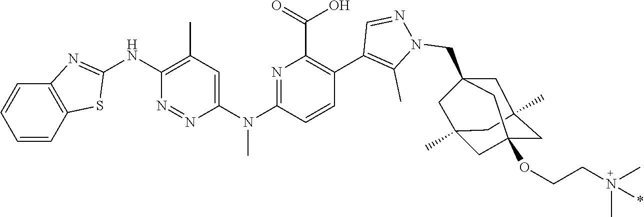 | P30a |
| 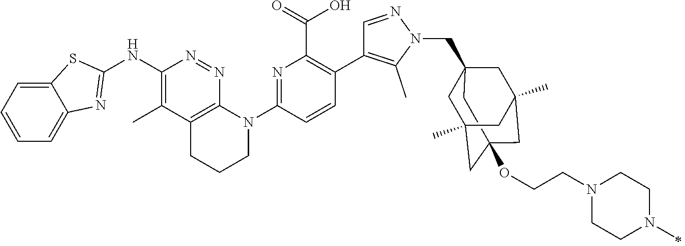 | P31a |
| 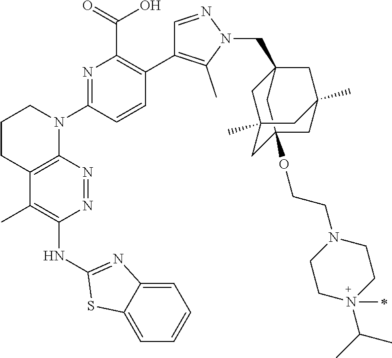 | P32a |
| 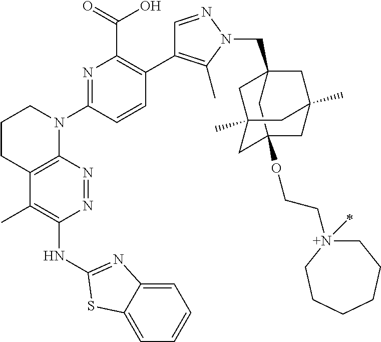 | P33a |
| 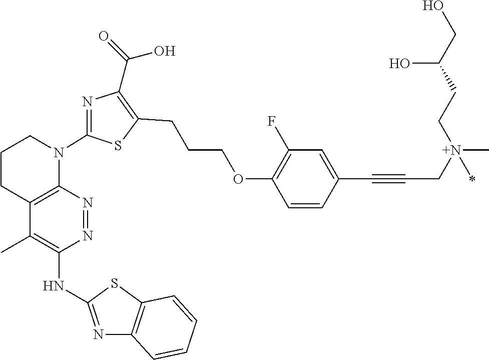 | P34a |
| 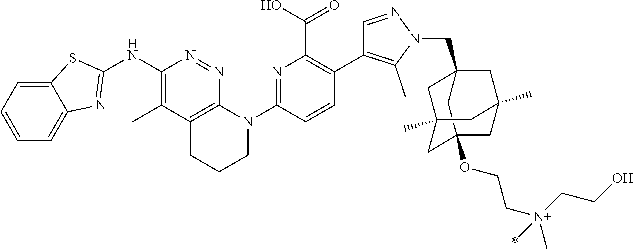 | P35a |
| 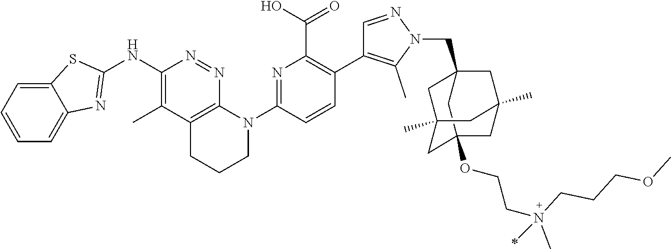 | P36a |
| 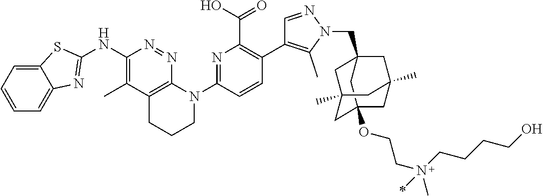 | P37a |
| 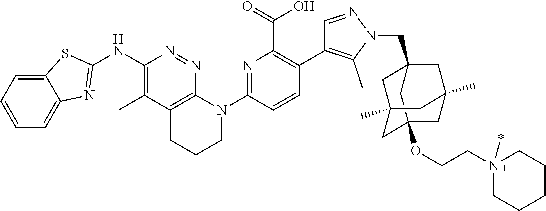 | P38a |
| 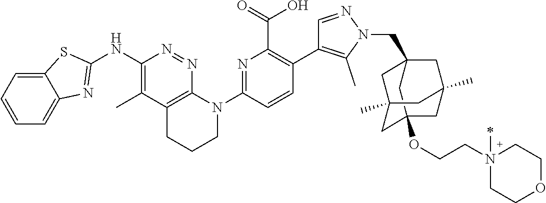 | P39a |
| 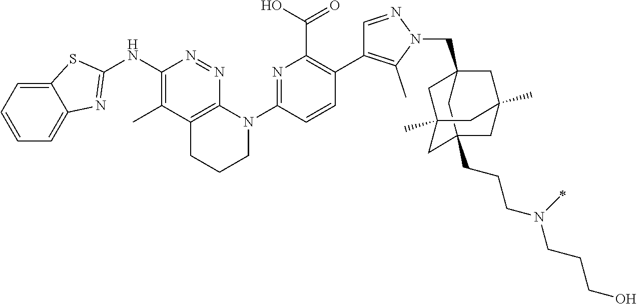 | P40a |
| 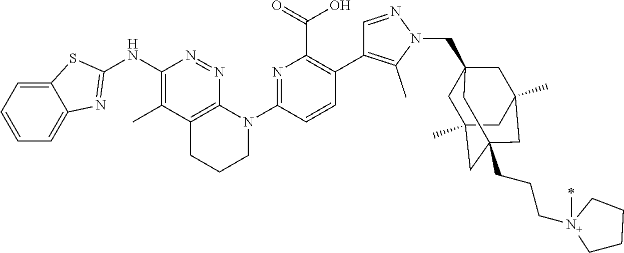 | P41a |
| 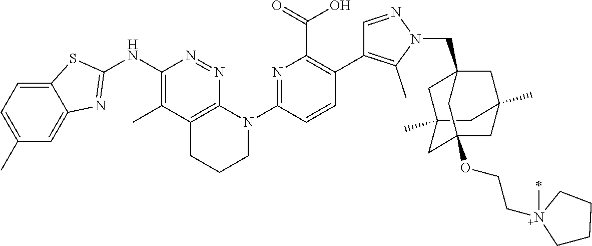 | P42a |
| 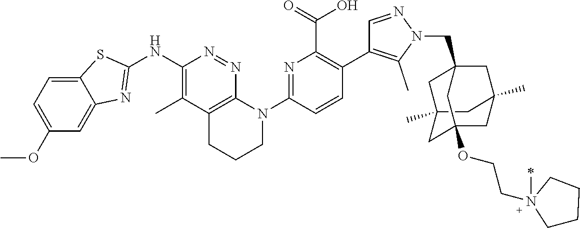 | P43a |
| 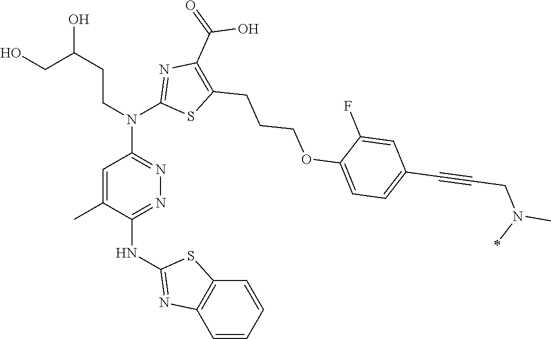 | P44a |
| 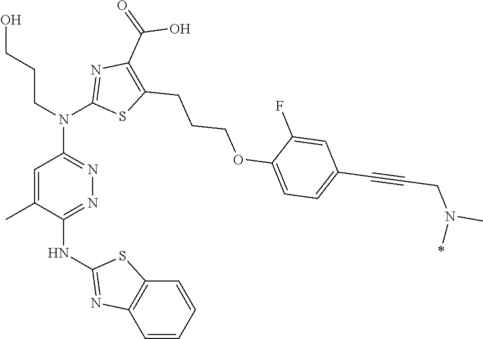 | P45a |
| 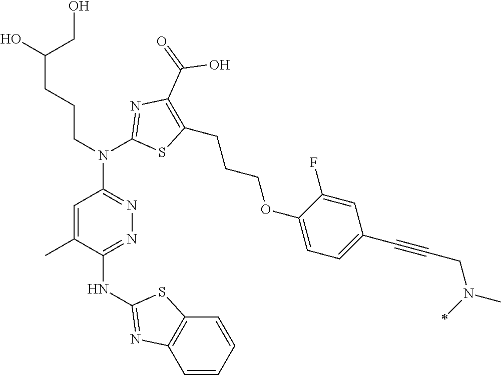 | P46a |
| 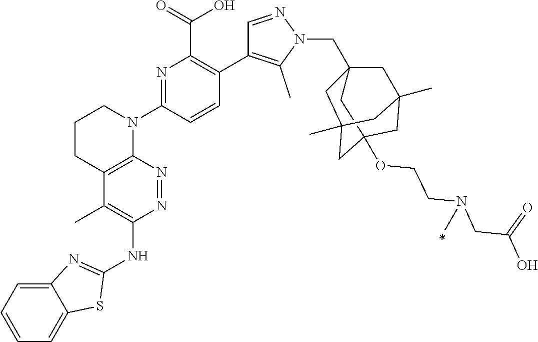 [SMILES: Cc1c(Nc2nc3ccccc3s2)nnc2c1CCCN2c1ccc(-c2cnn(CC34CC5(C)CC(C)(C3)CC(OCCN(C)CC(=O)O)(C5)C4)c2C)c(C(=O)O)n1] | P47a |
| 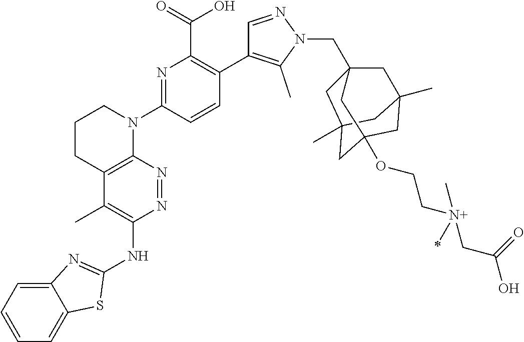 | P48a |
| 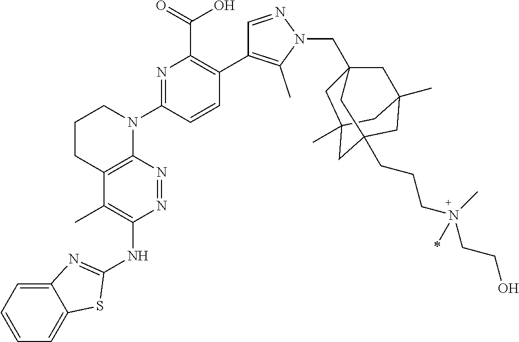 | P49a |
| 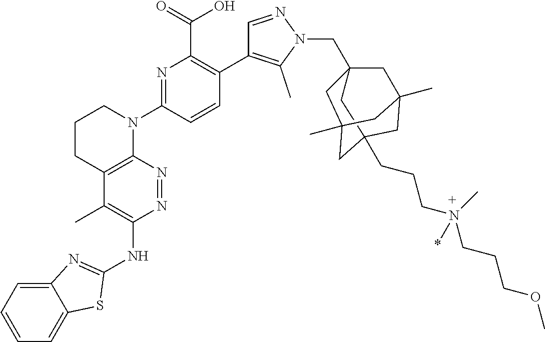 | P50a |
| 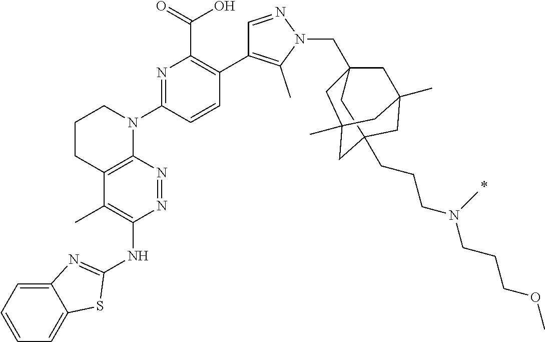 [SMILES: COCCCN(C)CCCC12CC3(C)CC(C)(C1)CC(Cn1ncc(-c4ccc(N5CCCc6c5nnc(Nc5nc7ccccc7s5)c6C)nc4C(=O)O)c1C)(C3)C2] | P51a |
| 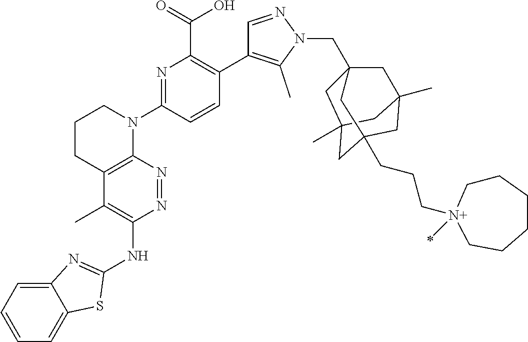 | P52a |
| 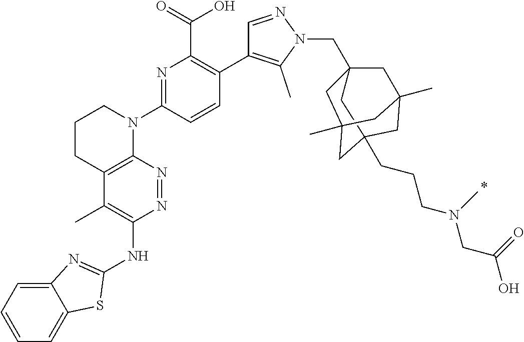 [SMILES: Cc1c(Nc2nc3ccccc3s2)nnc2c1CCCN2c1ccc(-c2cnn(CC34CC5(C)CC(C)(CC(CCCN(C)CC(=O)O)(C5)C3)C4)c2C)c(C(=O)O)n1] | P53a |
| 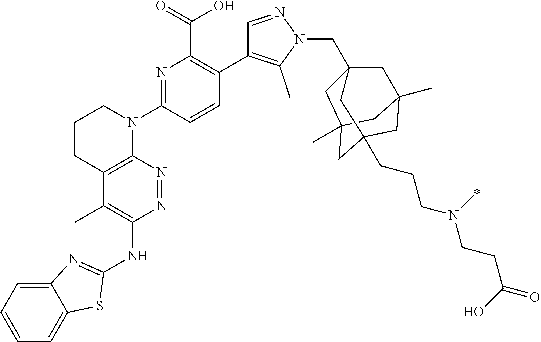 | P54a |
| 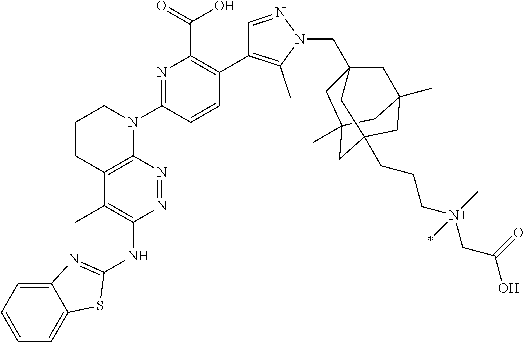 | P55a |
| 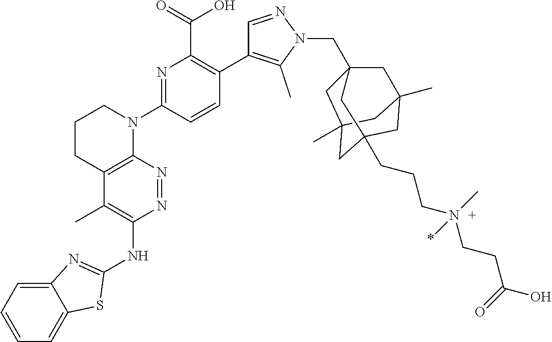 | P56a |
| 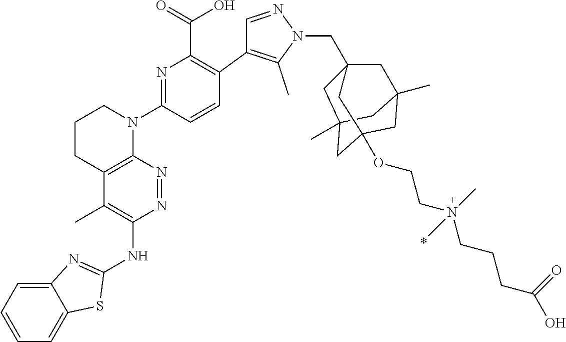 | P57a |
| 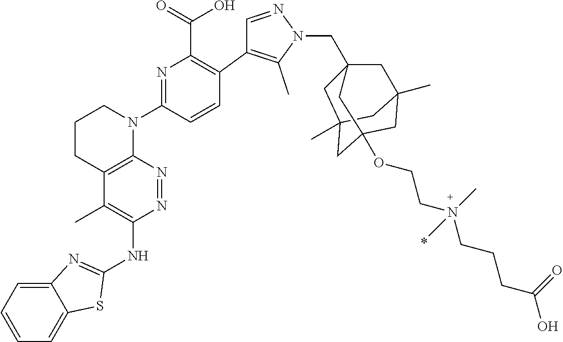 | P58a |
| 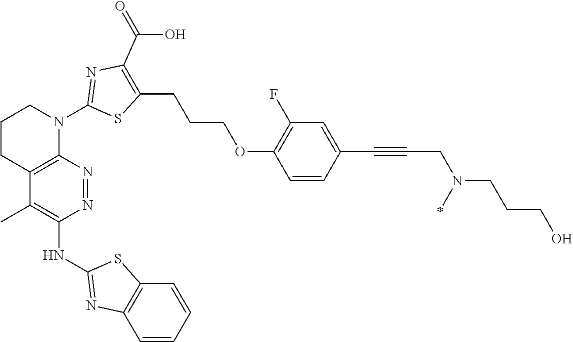 | P59a |
| 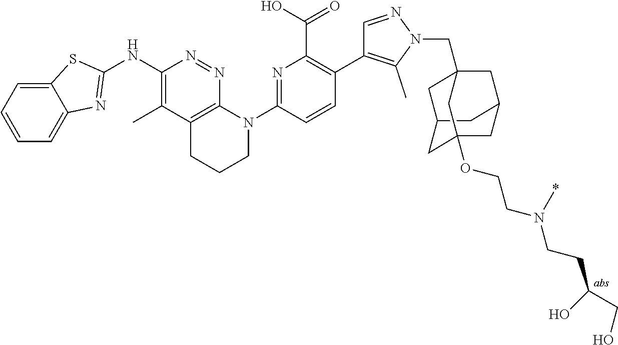 | P60a |
| 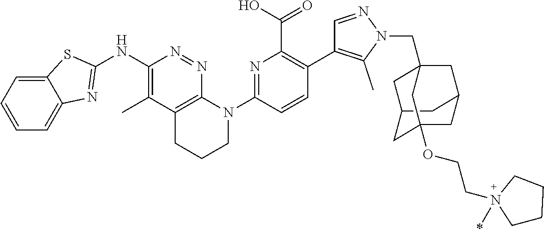 | P61a |
| 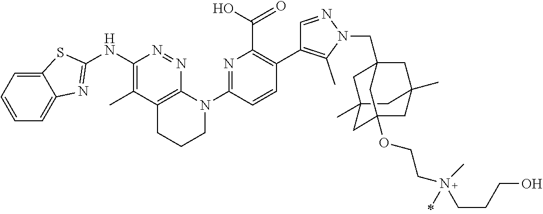 | P62a |
| 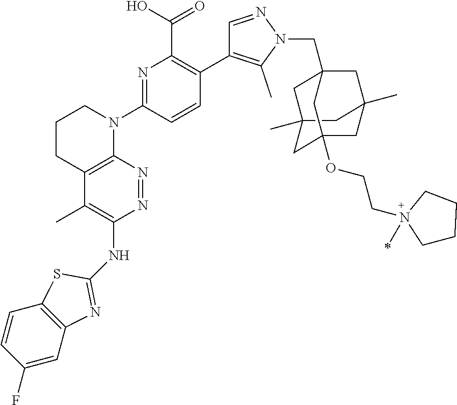 | P63a |
| 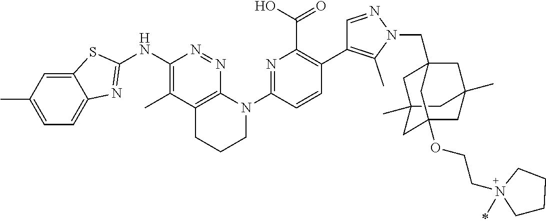 | P64a |
| 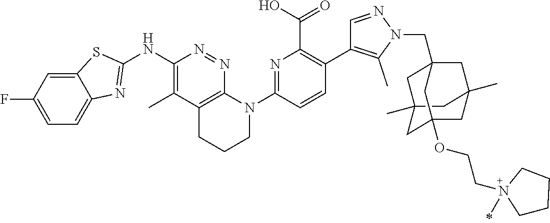 | P65a |
| 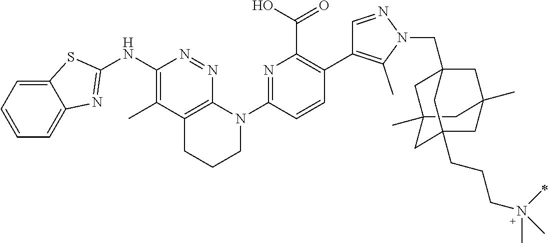 | P66a |
| 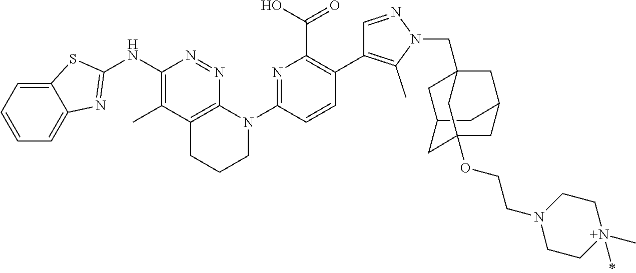 | P67a |
| 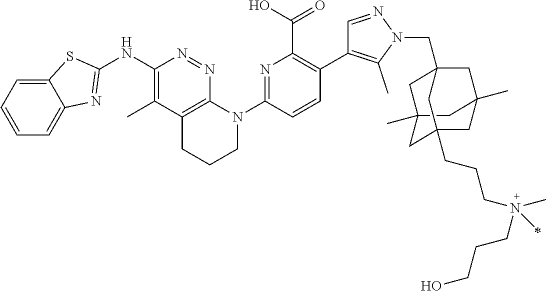 | P68a |
| 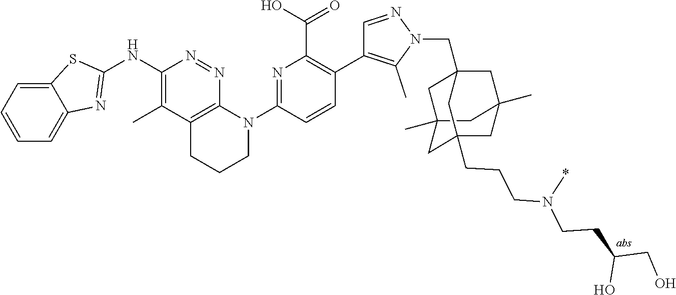 [SMILES: *N(CCCC12CC3(C)CC(C)(C1)CC(Cn1ncc(-c4ccc(N5CCCc6c5nnc(Nc5nc7ccccc7s5)c6C)nc4C(=O)O)c1C)(C3)C2)CC[C@H](O)CO] | P69a |
| 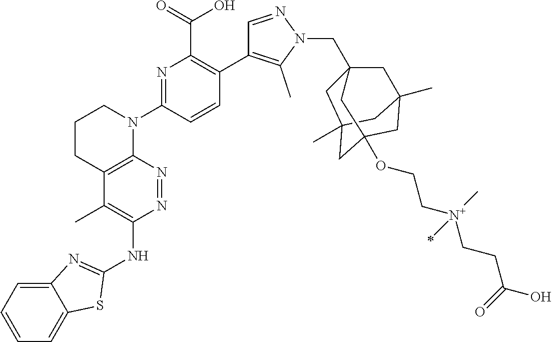 | P70a |
| 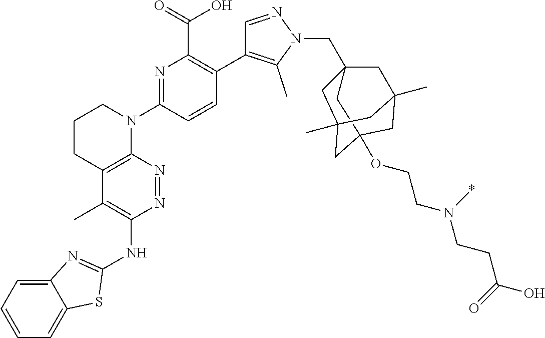 [SMILES: *N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CCC(=O)O] | P71a |
| 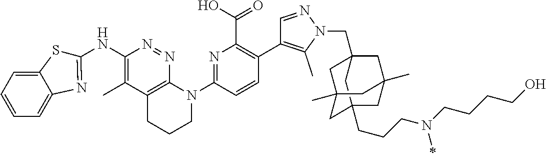 | P72a |
| 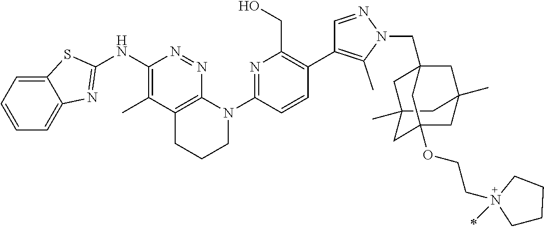 | P73a |
| 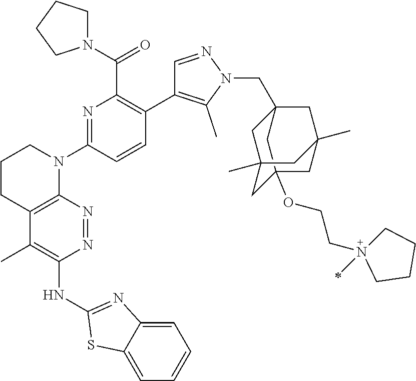 | P74a |
| 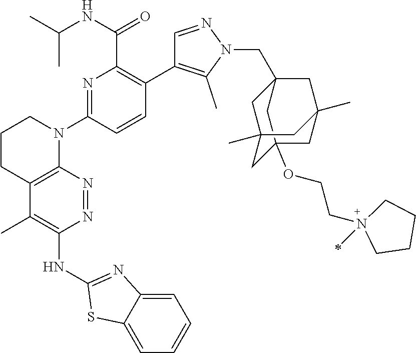 | P75a |
| 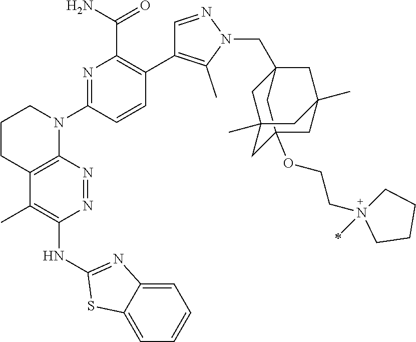 | P75a |

wherein [symbol] represents a bond to the linker.

In some embodiments, -(L-D) is formed from a compound selected from Table B or an enantiomer, a diastereoisomer, and/or a pharmaceutically acceptable salt thereof. In some embodiments, the maleimide group

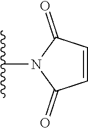 [SMILES: *N1C(=O)C=CC1=O]

in the compound of Table B form a covalent bond with the antibody or antigen-binding fragment thereof (Ab) to form the ADC compound of formula (1) comprising a

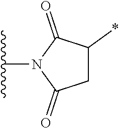 [SMILES: *C1CC(=O)N(*)C1=O]

moiety, wherein * indicates the connection point to Ab. For compounds in Table A1, Table A2, Table B and Table 1, depending on their electronic charge, these compounds can contain one pharmaceutically acceptable monovalent anionic counterion M₁⁻. In some embodiments, the monovalent anionic counterion M₁⁻ can be selected from bromide, chloride, iodide, acetate, trifluoroacetate, benzoate, mesylate, tosylate, triflate, formate, or the like. In some embodiments, the monovalent anionic counterion M₁⁻ is trifluoroacetate or formate.

TABLE B
Exemplary Linker Drug Groups

| Name | Linker Payload Structure |
|---|---|
| L1A-P1 | 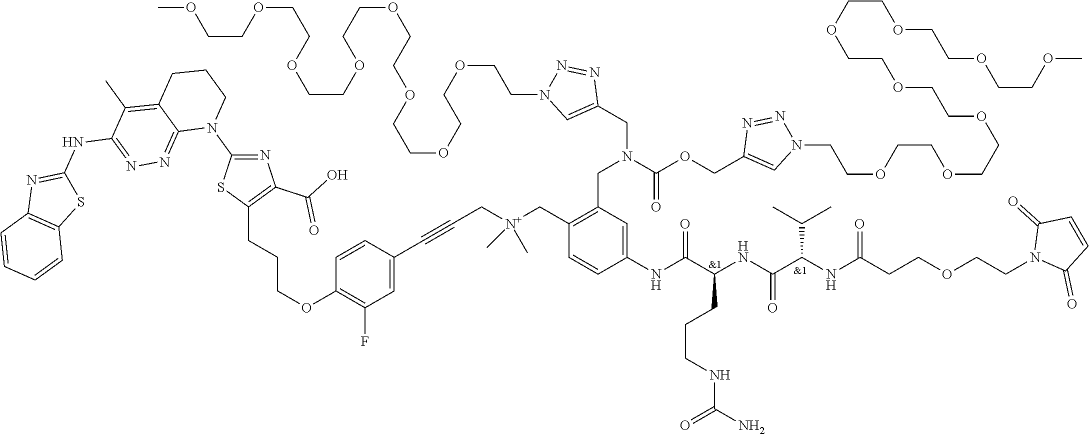 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](NC(=O)CCOCCN3C(=O)C=CC3=O)C(C)C)ccc2C[N+](C)(C)CC#Cc2ccc(OCCCc3sc(N4CCCc5c4nnc(Nc4nc6ccccc6s4)c5C)nc3C(=O)O)c(F)c2)nn1] |
| L1A-P2 | 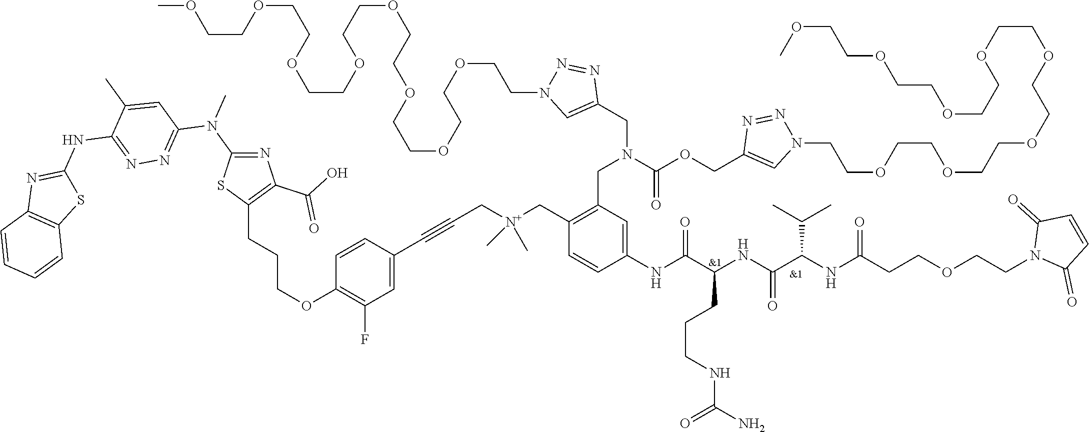 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](NC(=O)CCOCCN3C(=O)C=CC3=O)C(C)C)ccc2C[N+](C)(C)CC#Cc2ccc(OCCCc3sc(N(C)c4cc(C)c(Nc5nc6ccccc6s5)nn4)nc3C(=O)O)c(F)c2)nn1] |
| L1C-P3 | 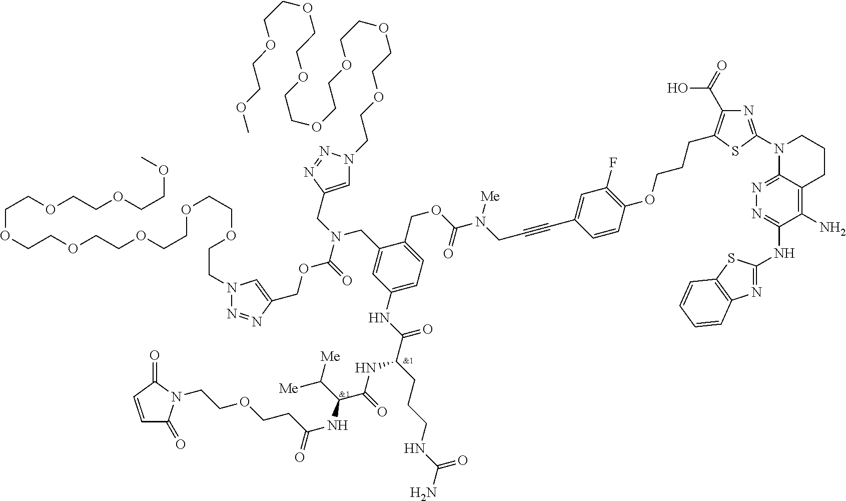 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](NC(=O)CCOCCN3C(=O)C=CC3=O)C(C)C)ccc2COC(=O)N(C)CC#Cc2ccc(OCCCc3sc(N4CCCc5c4nnc(Nc4nc6ccccc6s4)c5N)nc3C(=O)O)c(F)c2)nn1] |
| L3A-P1 | 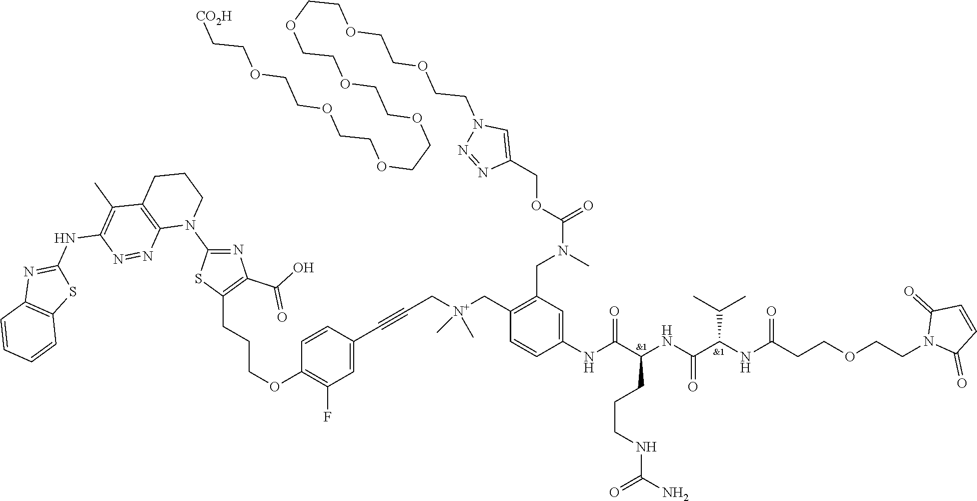 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)OCc3cn(CCOCCOCCOCCOCCOCCOCCOCCOCCC(=O)O)nn3)cc2F)s1] |
| L3C-P4 | 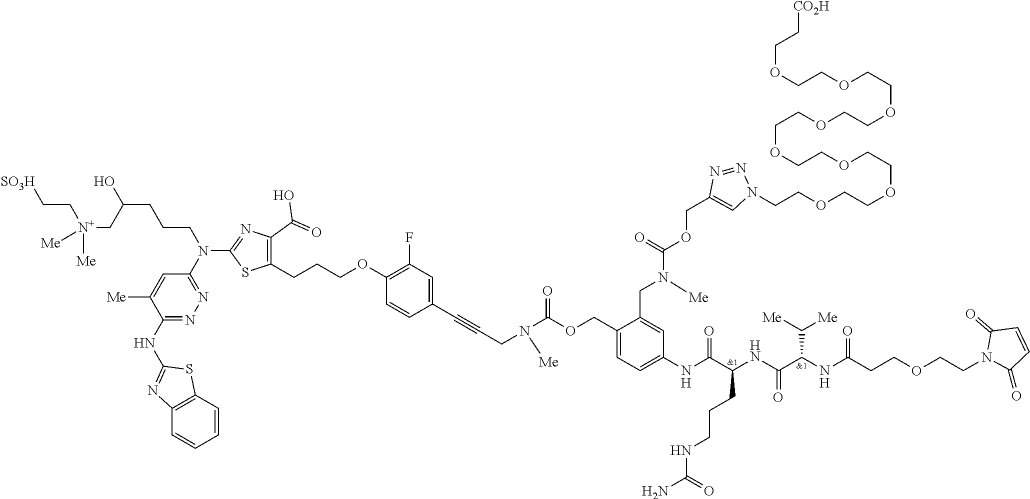 [SMILES: Cc1cc(N(CCCC(O)C[N+](C)(C)CCS(=O)(=O)O)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4CN(C)C(=O)OCc4cn(CCOCCOCCOCCOCCOCCOCCOCCOCCC(=O)O)nn4)cc3F)s2)nnc1Nc1nc2ccccc2s1] |
| L3C-P5 | 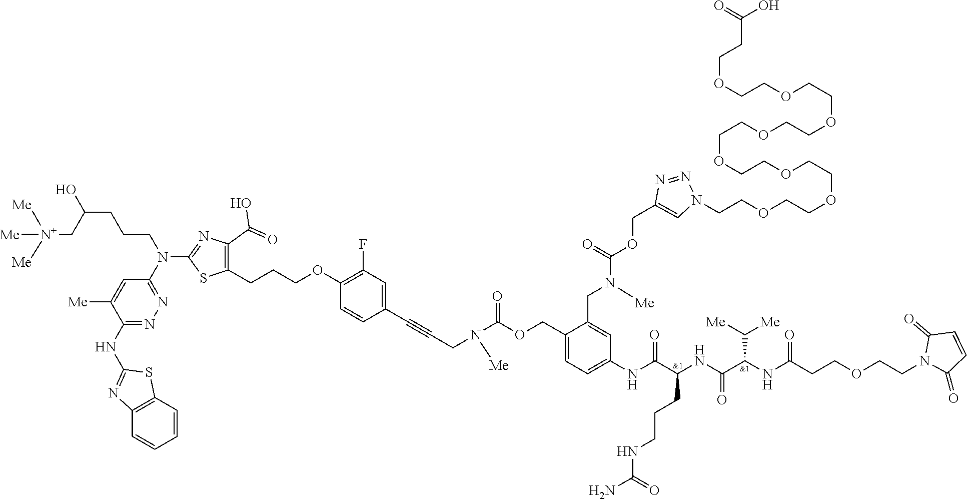 [SMILES: Cc1cc(N(CCCC(O)C[N+](C)(C)C)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4CN(C)C(=O)OCc4cn(CCOCCOCCOCCOCCOCCOCCOCCOCCC(=O)O)nn4)cc3F)s2)nnc1Nc1nc2ccccc2s1] |
| L3C-P3 | 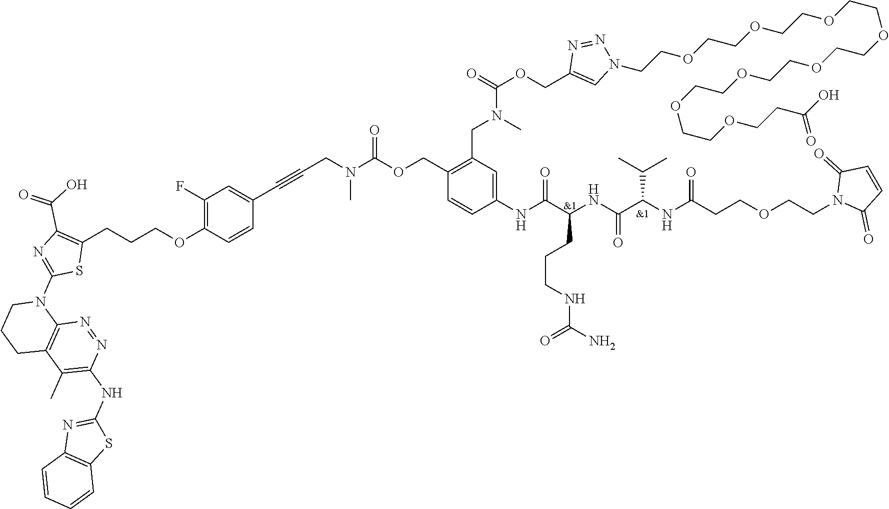 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3CN(C)C(=O)OCc3cn(CCOCCOCCOCCOCCOCCOCCOCCOCCC(=O)O)nn3)cc2F)s1] |
| L4A-P1 | 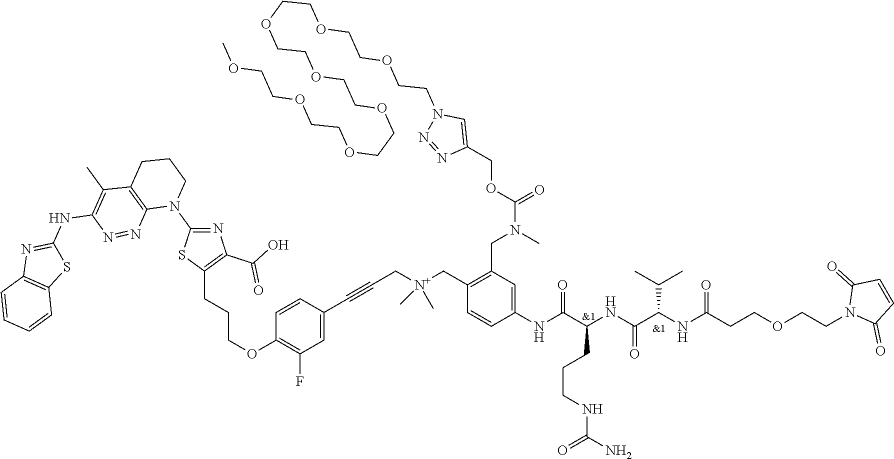 [SMILES: COCCOCCOCCOCCOCCOCCOCCOCCn1cc(COC(=O)N(C)Cc2cc(NC(=O)[C@H](CCCNC(N)=O)NC(=O)[C@@H](NC(=O)CCOCCN3C(=O)C=CC3=O)C(C)C)ccc2C[N+](C)(C)CC#Cc2ccc(OCCCc3sc(N4CCCc5c4nnc(Nc4nc6ccccc6s4)c5C)nc3C(=O)O)c(F)c2)nn1] |
| L7A-P1 | 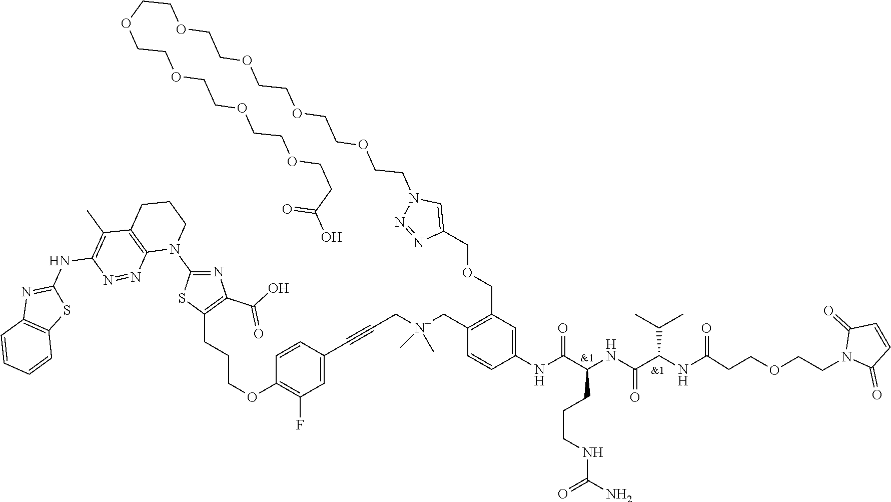 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3COCc3cn(CCOCCOCCOCCOCCOCCOCCOCCOCCC(=O)O)nn3)cc2F)s1] |
| L7A-P2 | 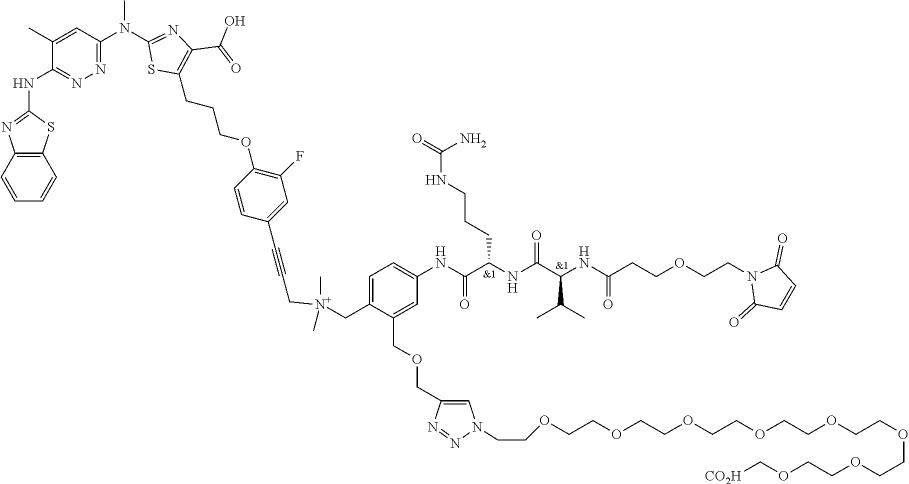 [SMILES: Cc1cc(N(C)c2nc(C(=O)O)c(CCCOc3ccc(C#CC[N+](C)(C)Cc4ccc(NC(=O)[C@H](CCCNC(N)=O)NC(=O)[C@@H](NC(=O)CCOCCN5C(=O)C=CC5=O)C(C)C)cc4COCc4cn(CCOCCOCCOCCOCCOCCOCCOCCOCC(=O)O)nn4)cc3F)s2)nnc1Nc1nc2ccccc2s1] |
| L7C-P3 | 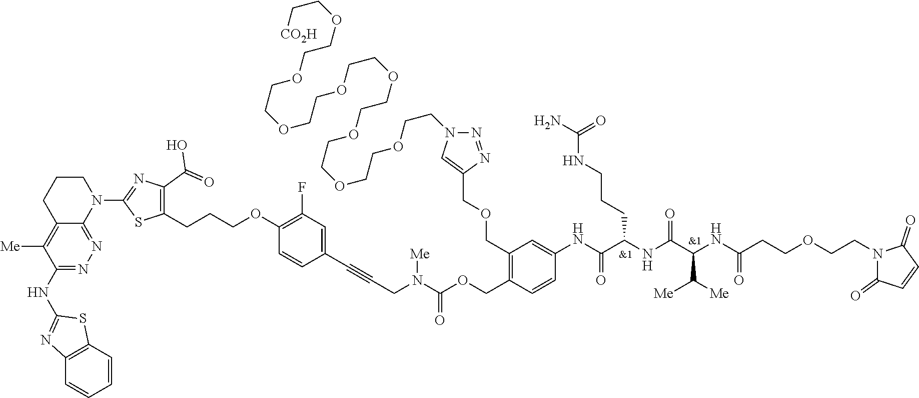 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3COCc3cn(CCOCCOCCOCCOCCOCCOCCOCCOCCC(=O)O)nn3)cc2F)s1] |
| L7C-P6 | 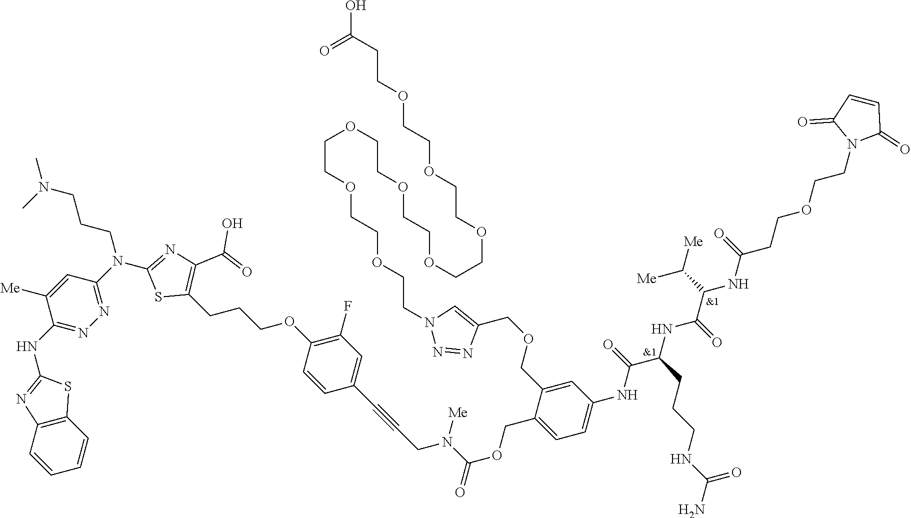 |
| L7C-P7 | 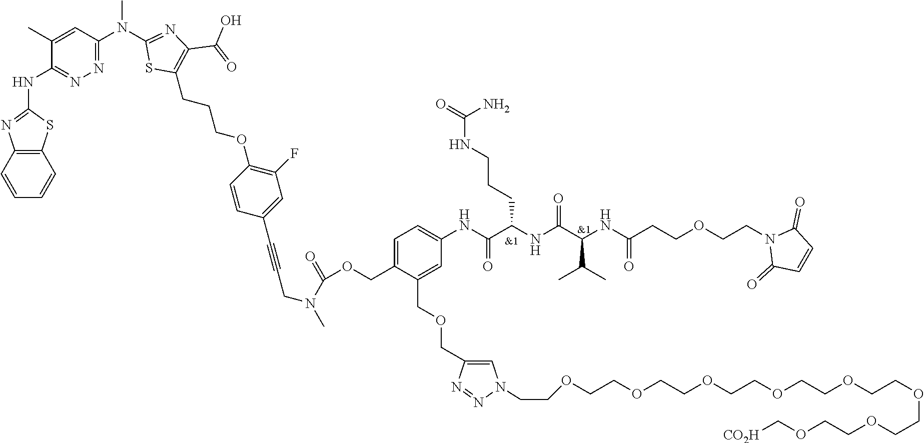 [SMILES: Cc1cc(N(C)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4COCc4cn(CCOCCOCCOCCOCCOCCOCCOCCOCC(=O)O)nn4)cc3F)s2)nnc1Nc1nc2ccccc2s1] |
| L8A-P1 | 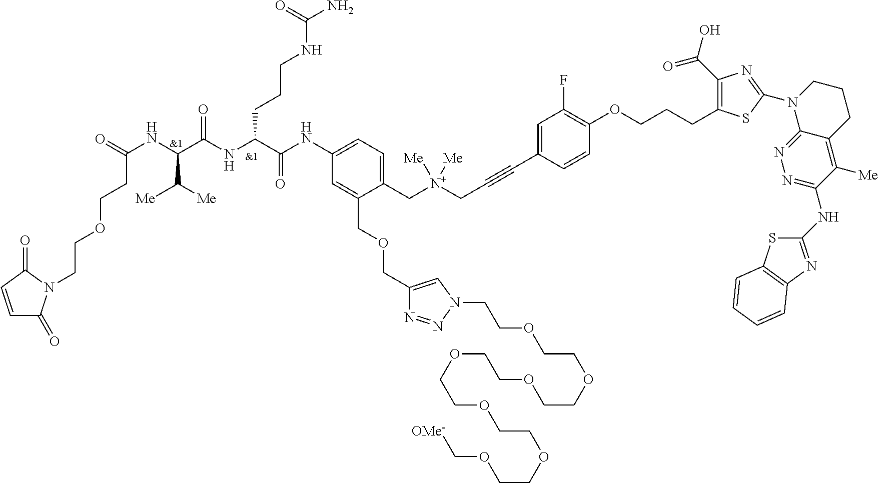 |
| L8C-P7 | 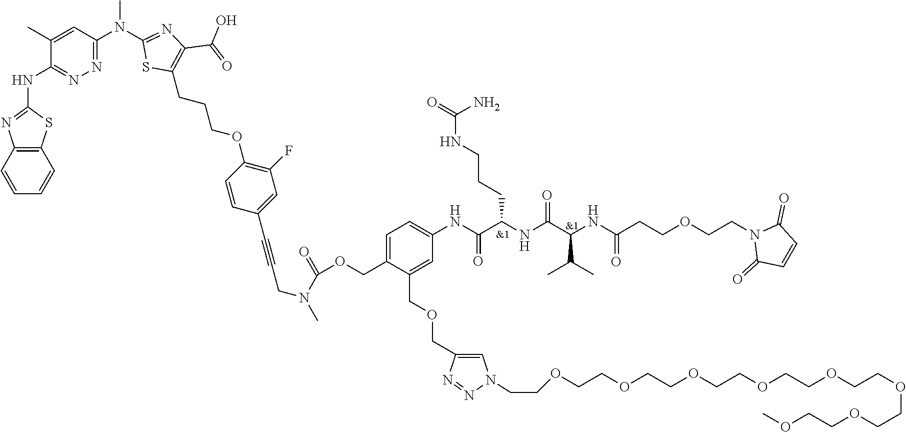 [SMILES: COCCOCCOCCOCCOCCOCCOCCOCCn1cc(COCc2cc(NC(=O)[C@H](CCCNC(N)=O)NC(=O)[C@@H](NC(=O)CCOCCN3C(=O)C=CC3=O)C(C)C)ccc2COC(=O)N(C)CC#Cc2ccc(OCCCc3sc(N(C)c4cc(C)c(Nc5nc6ccccc6s5)nn4)nc3C(=O)O)c(F)c2)nn1] |
| L9A-P8 | 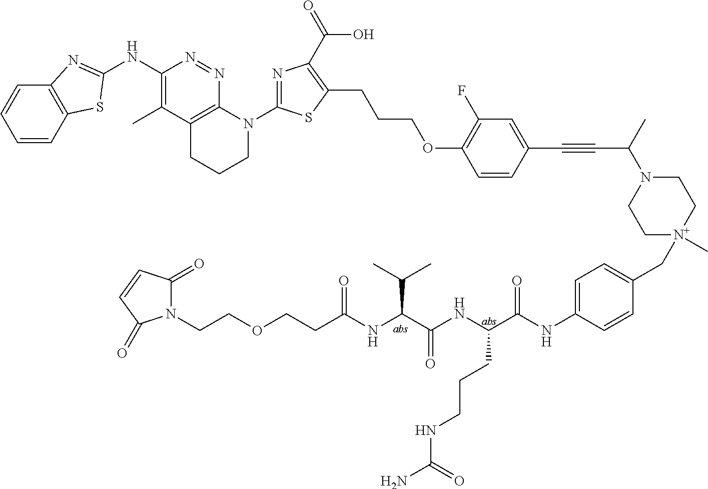 [SMILES: Cc1c(Nc2nc3ccccc3s2)nnc2c1CCCN2c1nc(C(=O)O)c(CCCOc2ccc(C#CC(C)N3CC[N+](C)(Cc4ccc(NC(=O)[C@H](CCCNC(N)=O)NC(=O)[C@@H](NC(=O)CCOCCN5C(=O)C=CC5=O)C(C)C)cc4)CC3)cc2F)s1] |
| L9A-P9 | 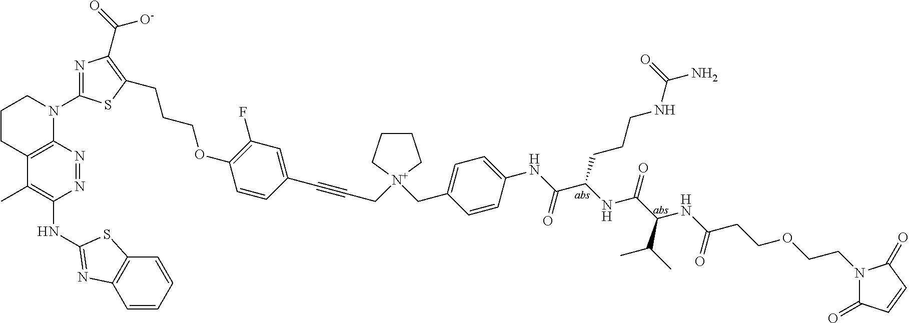 [SMILES: Cc1c(Nc2nc3ccccc3s2)nnc2c1CCCN2c1nc(C(=O)[O-])c(CCCOc2ccc(C#CC[N+]3(Cc4ccc(NC(=O)[C@H](CCCNC(N)=O)NC(=O)[C@@H](NC(=O)CCOCCN5C(=O)C=CC5=O)C(C)C)cc4)CCCC3)cc2F)s1] |
| L9A-P10 | 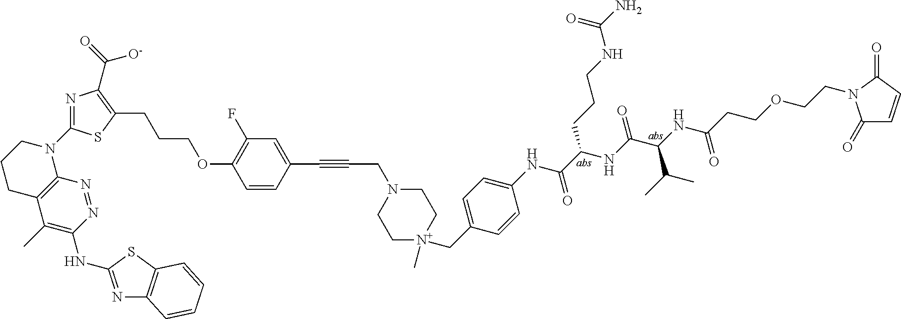 [SMILES: Cc1c(Nc2nc3ccccc3s2)nnc2c1CCCN2c1nc(C(=O)[O-])c(CCCOc2ccc(C#CCN3CC[N+](C)(Cc4ccc(NC(=O)[C@H](CCCNC(N)=O)NC(=O)[C@@H](NC(=O)CCOCCN5C(=O)C=CC5=O)C(C)C)cc4)CC3)cc2F)s1] |
| L9A-P11 | 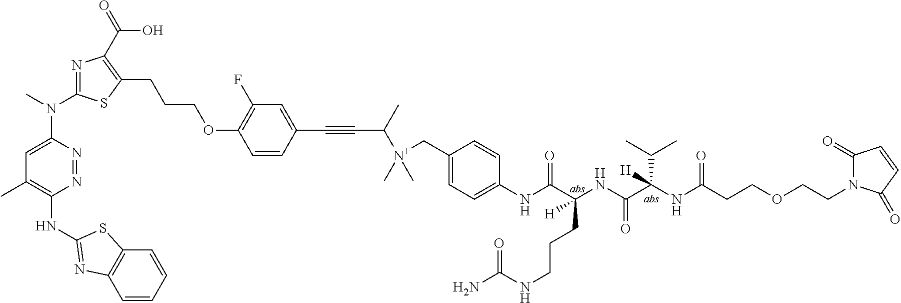 [SMILES: Cc1cc(N(C)c2nc(C(=O)O)c(CCCOc3ccc(C#CC(C)[N+](C)(C)Cc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P12 | 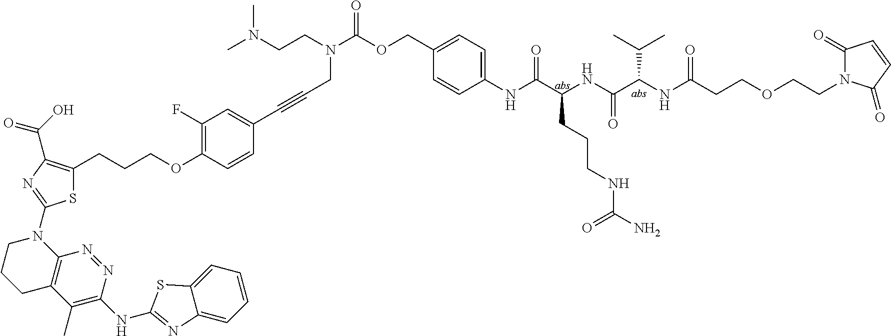 [SMILES: Cc1c(Nc2nc3ccccc3s2)nnc2c1CCCN2c1nc(C(=O)O)c(CCCOc2ccc(C#CCN(CCN(C)C)C(=O)OCc3ccc(NC(=O)[C@H](CCCNC(N)=O)NC(=O)[C@@H](NC(=O)CCOCCN4C(=O)C=CC4=O)C(C)C)cc3)cc2F)s1] |
| L9A-P13 | 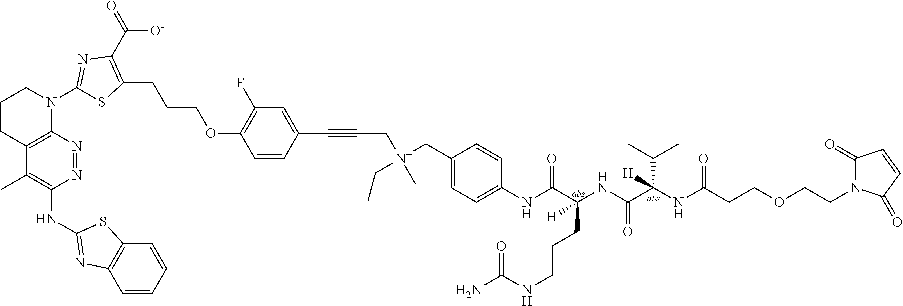 [SMILES: CC[N+](C)(CC#Cc1ccc(OCCCc2sc(N3CCCc4c3nnc(Nc3nc5ccccc5s3)c4C)nc2C(=O)[O-])c(F)c1)Cc1ccc(NC(=O)[C@H](CCCNC(N)=O)NC(=O)[C@@H](NC(=O)CCOCCN2C(=O)C=CC2=O)C(C)C)cc1] |
| L9A-P14 | 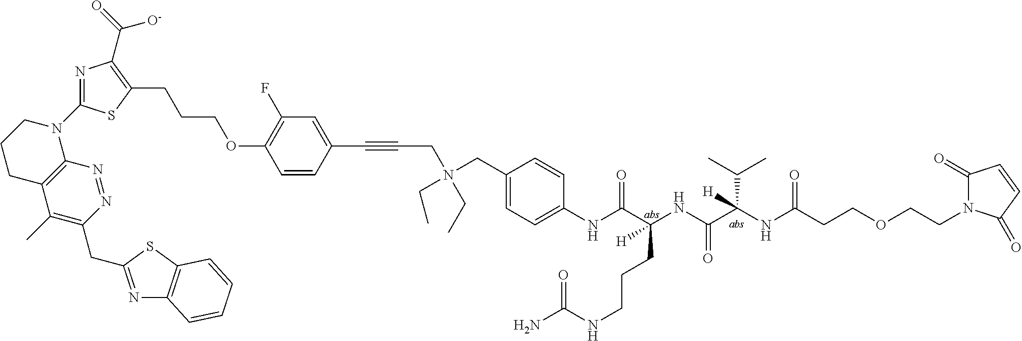 |
| L9A-P15 | 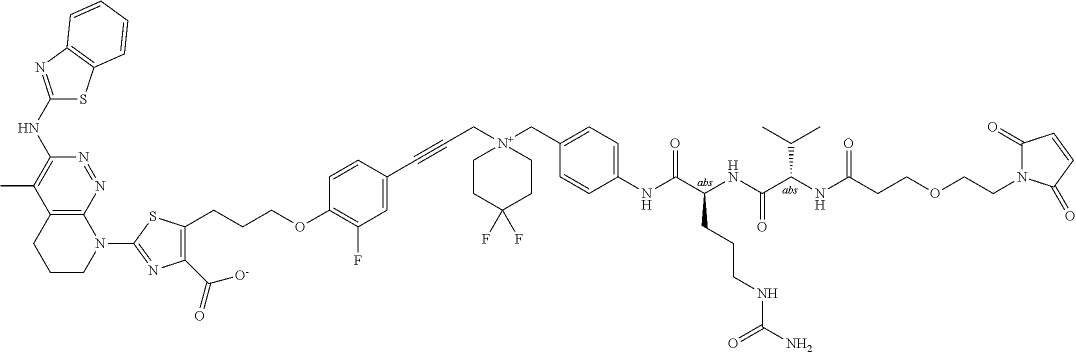 [SMILES: Cc1c(Nc2nc3ccccc3s2)nnc2c1CCCN2c1nc(C(=O)[O-])c(CCCOc2ccc(C#CC[N+]3(Cc4ccc(NC(=O)[C@H](CCCNC(N)=O)NC(=O)[C@@H](NC(=O)CCOCCN5C(=O)C=CC5=O)C(C)C)cc4)CCC(F)(F)CC3)cc2F)s1] |
| L9C-P16 | 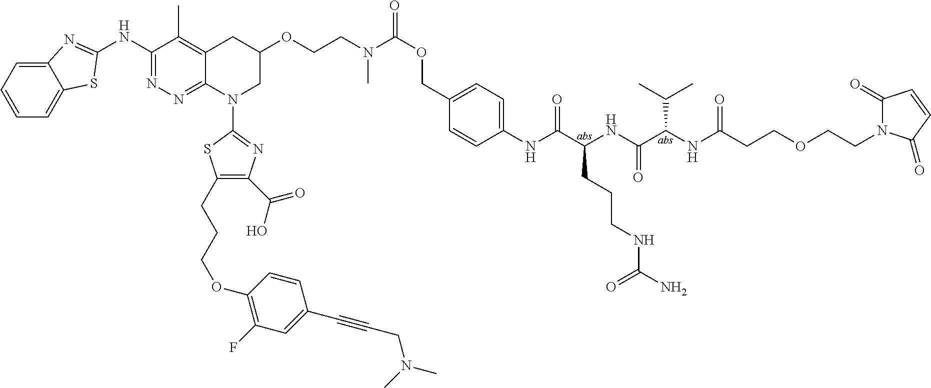 [SMILES: Cc1c(Nc2nc3ccccc3s2)nnc2c1CC(OCCN(C)C(=O)OCc1ccc(NC(=O)[C@H](CCCNC(N)=O)NC(=O)[C@@H](NC(=O)CCOCCN3C(=O)C=CC3=O)C(C)C)cc1)CN2c1nc(C(=O)O)c(CCCOc2ccc(C#CCN(C)C)cc2F)s1] |
| L9A-P1 | 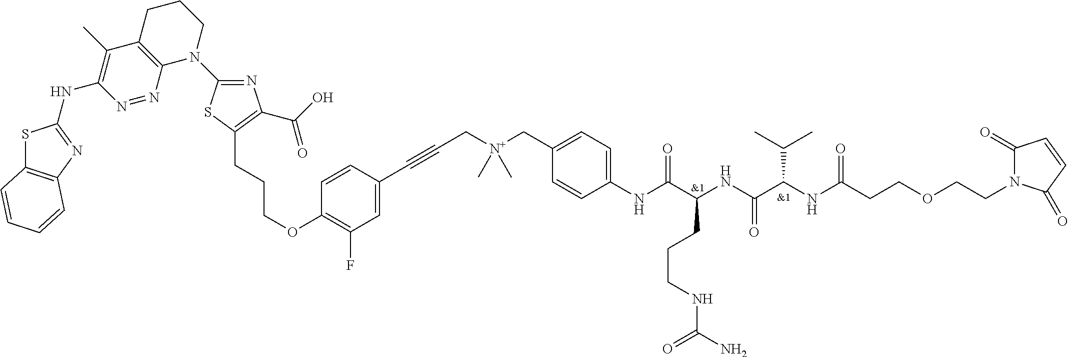 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3)cc2F)s1] |
| L9C-P17 | 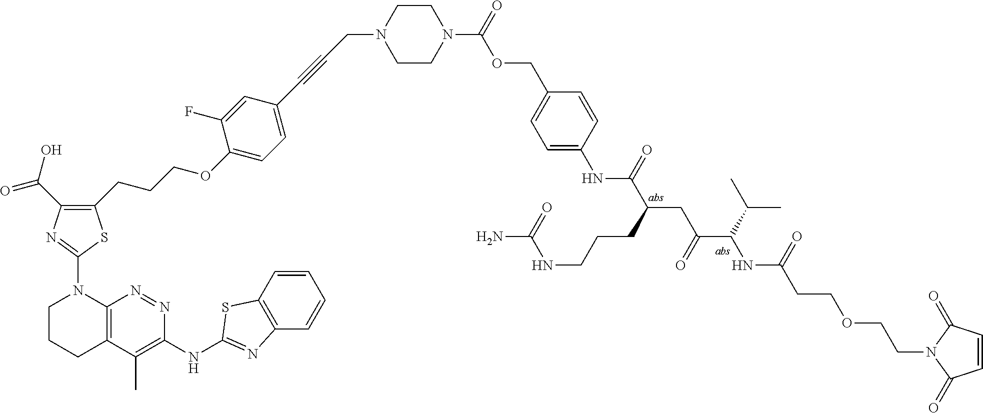 |
| L9A-P18 | 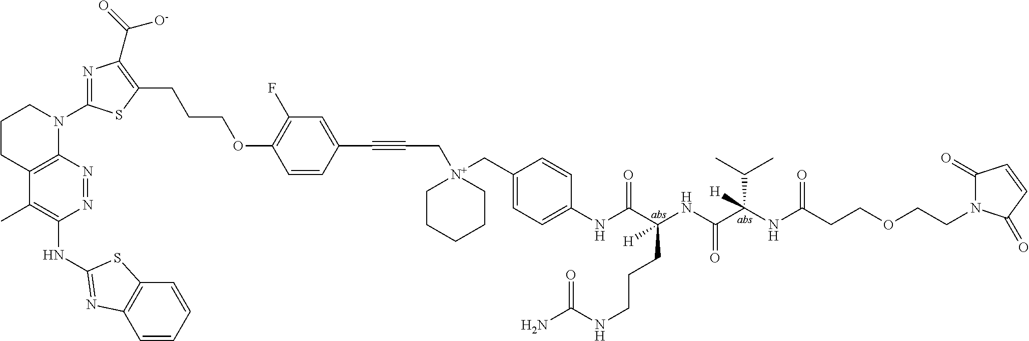 [SMILES: Cc1c(Nc2nc3ccccc3s2)nnc2c1CCCN2c1nc(C(=O)[O-])c(CCCOc2ccc(C#CC[N+]3(Cc4ccc(NC(=O)[C@H](CCCNC(N)=O)NC(=O)[C@@H](NC(=O)CCOCCN5C(=O)C=CC5=O)C(C)C)cc4)CCCCC3)cc2F)s1] |
| L9C-P19 | 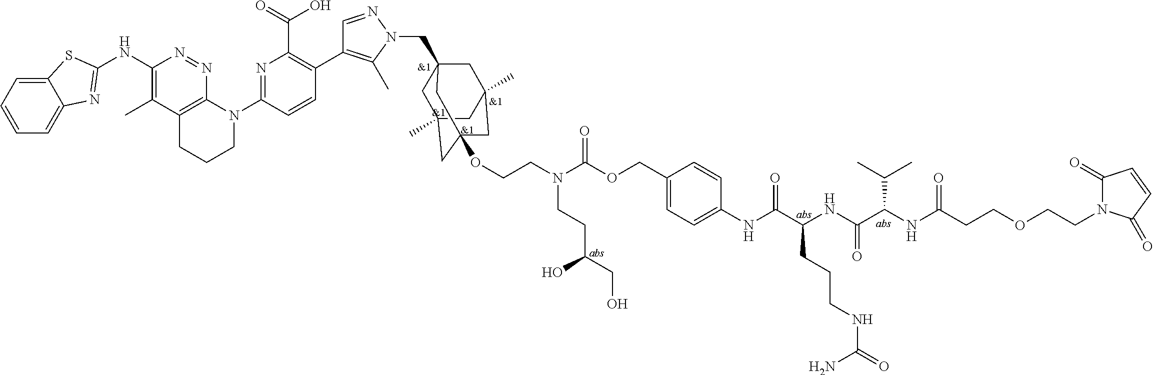 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P20 | 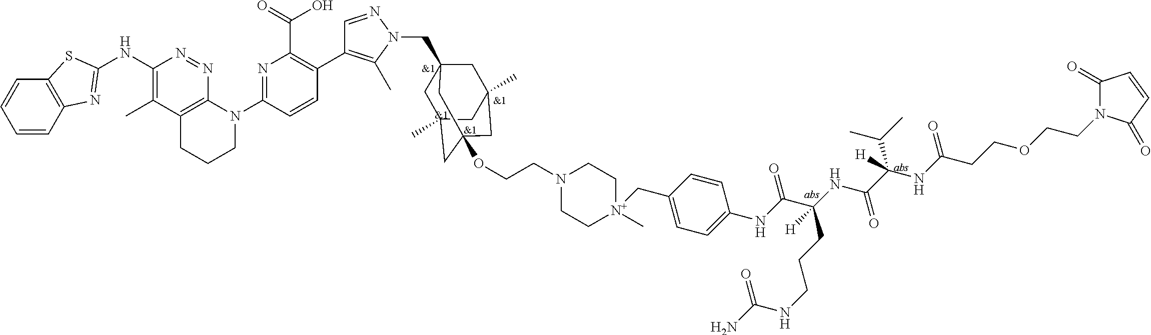 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CC[N+](C)(Cc6ccc(NC(=O)[C@H](CCCNC(N)=O)NC(=O)[C@@H](NC(=O)CCOCCN7C(=O)C=CC7=O)C(C)C)cc6)CC3)(C5)C4)c2C)c(C(=O)O)n1] |
| L9A-P21 | 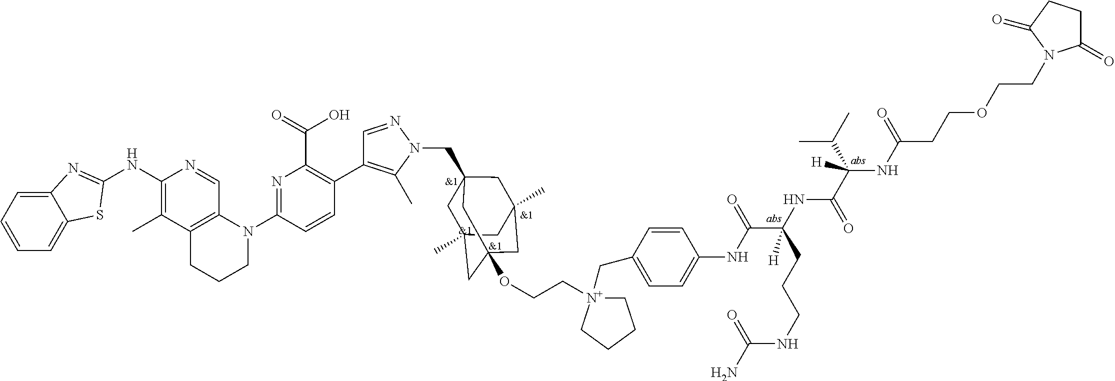 [SMILES: Cc1c(Nc2nc3ccccc3s2)ncc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)CCC7=O)C(C)C)cc6)CCCC3)(C5)C4)c2C)c(C(=O)O)n1] |
| L9C-P22 | 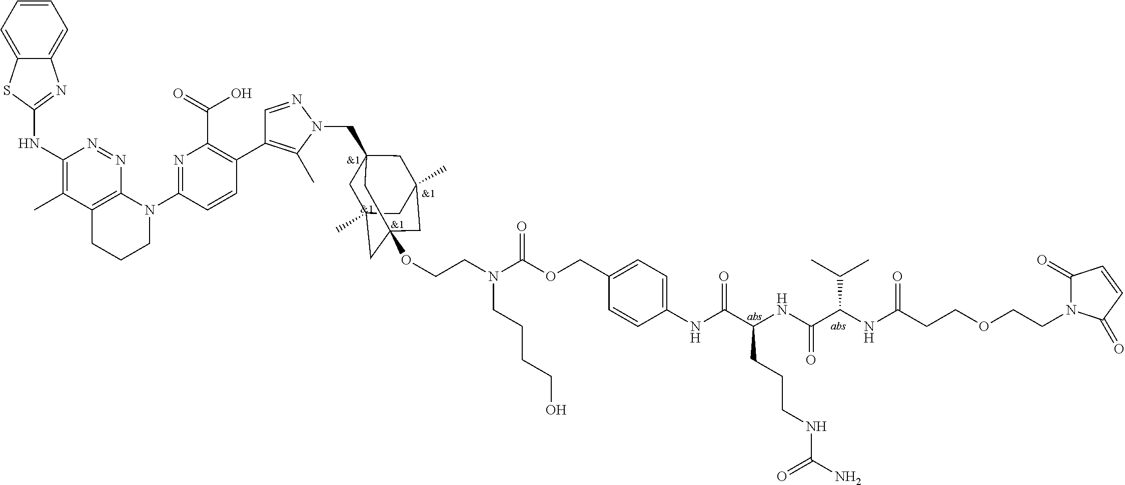 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CCCCO)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P23 | 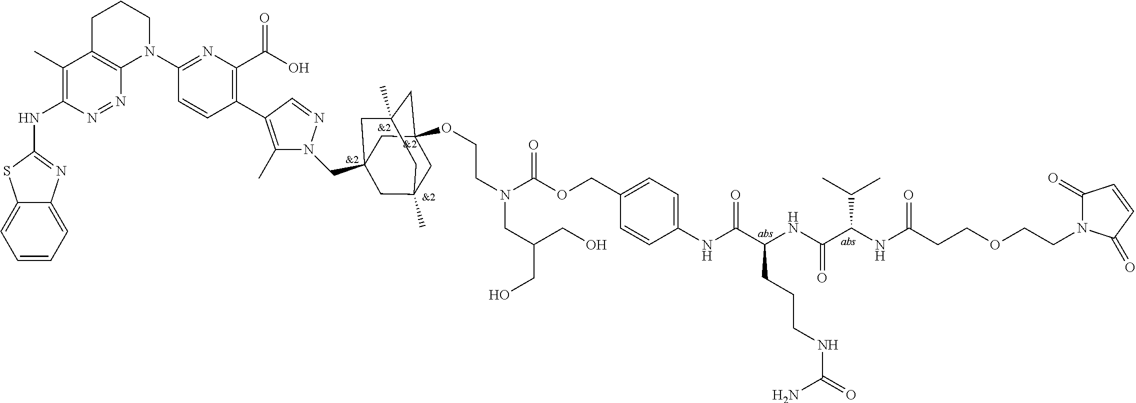 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CC(CO)CO)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P24 | 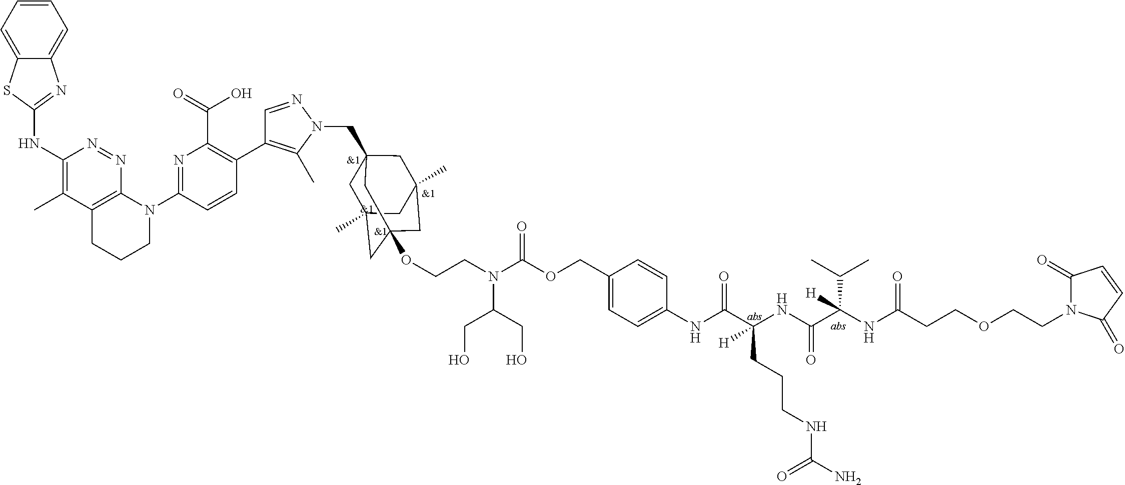 [SMILES: Cc1c(Nc2nc3ccccc3s2)nnc2c1CCCN2c1ccc(-c2cnn(C[C@]34C[C@]5(C)C[C@](C)(C3)C[C@@](OCCN(C(=O)OCc3ccc(NC(=O)[C@H](CCCNC(N)=O)NC(=O)[C@@H](NC(=O)CCOCCN6C(=O)C=CC6=O)C(C)C)cc3)C(CO)CO)(C5)C4)c2C)c(C(=O)O)n1] |
| L9A-P2 | 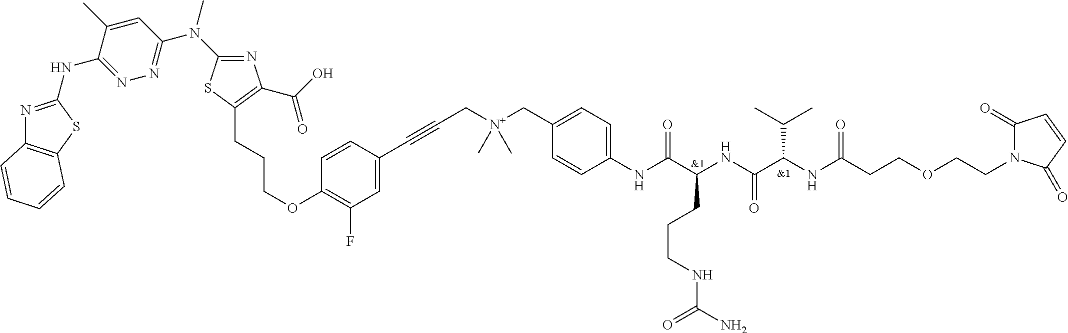 [SMILES: Cc1cc(N(C)c2nc(C(=O)O)c(CCCOc3ccc(C#CC[N+](C)(C)Cc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P25 | 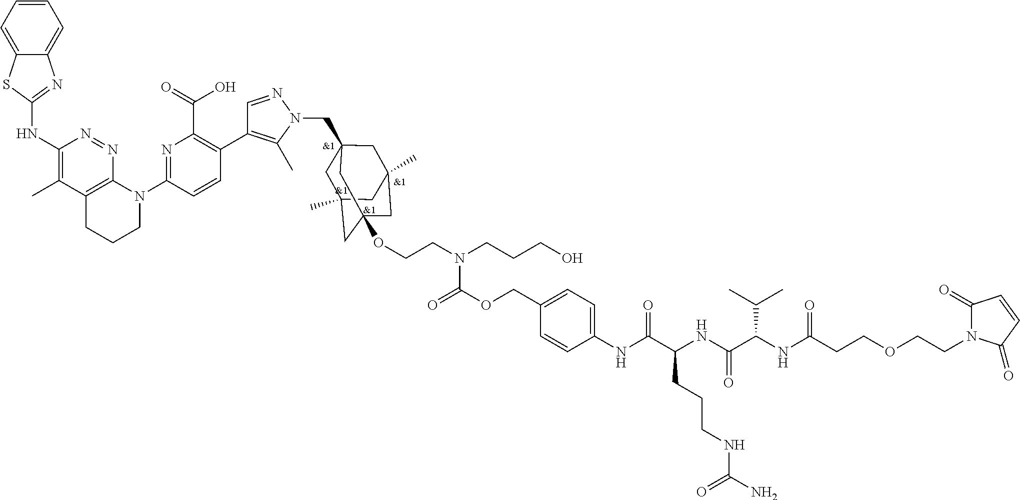 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P26 | 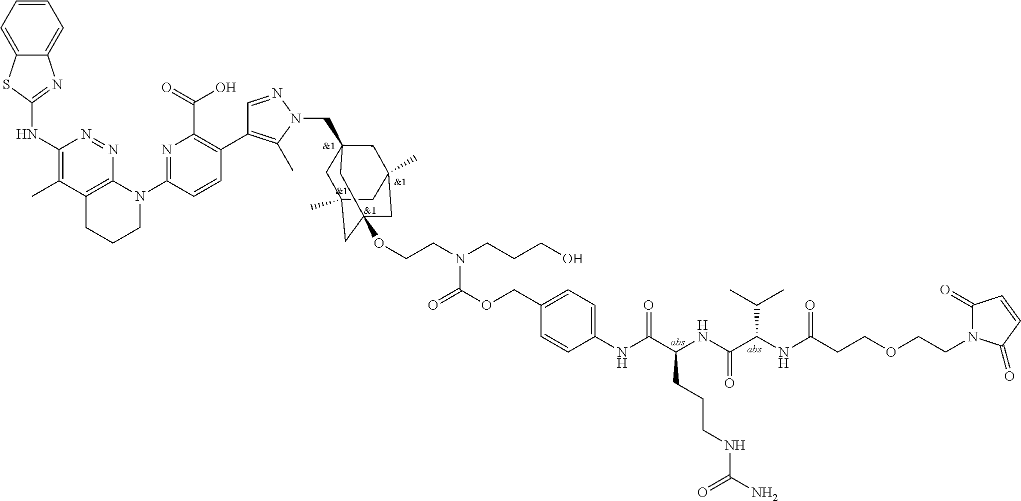 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P27 | 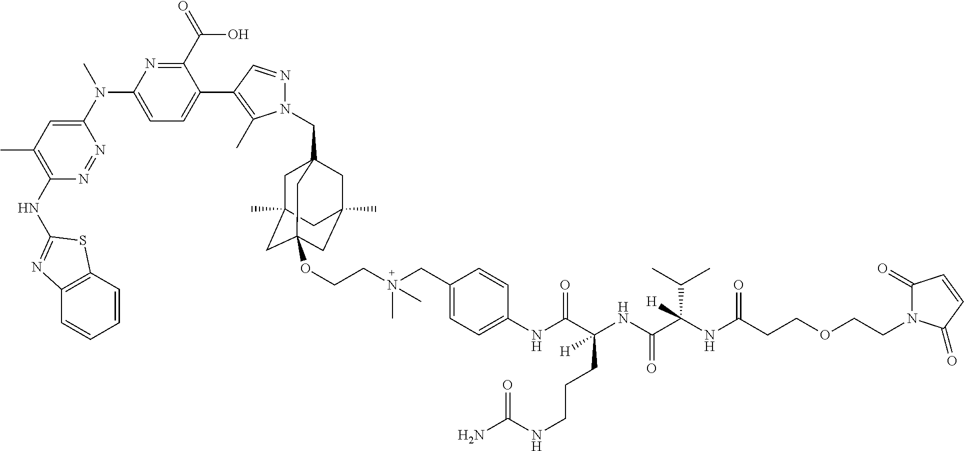 |
| L9A-P28 | 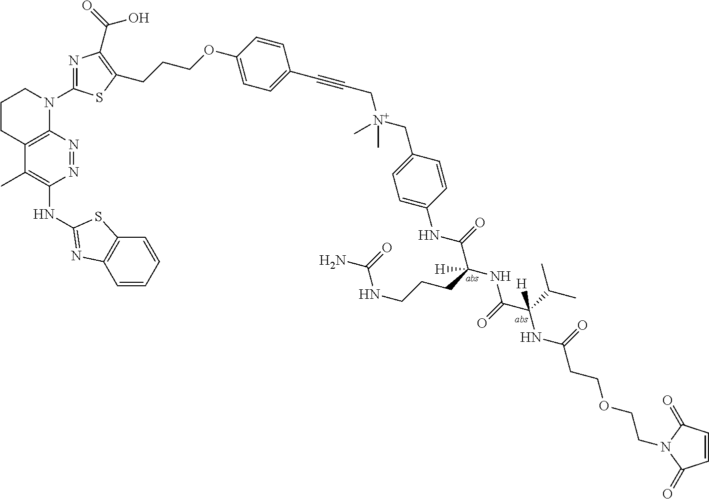 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3)cc2)s1] |
| L9C-P29 | 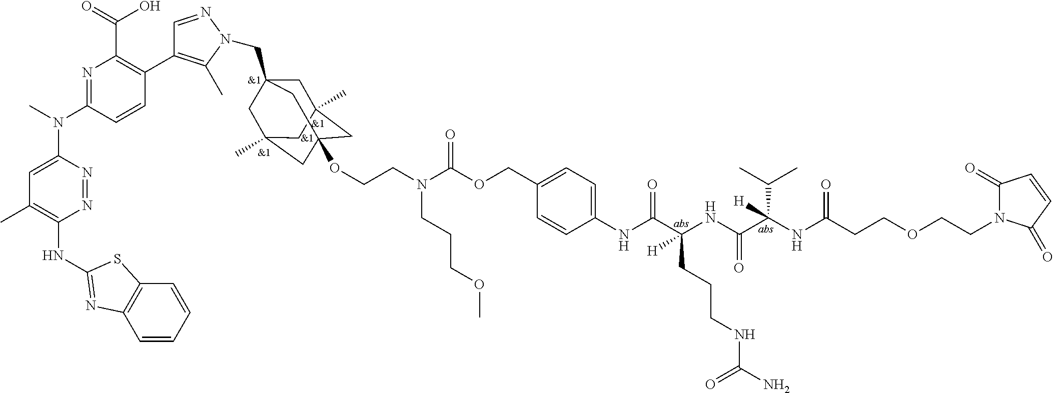 [SMILES: COCCCN(CCO[C@]12C[C@@]3(C)C[C@](C)(C[C@](Cn4ncc(-c5ccc(N(C)c6cc(C)c(Nc7nc8ccccc8s7)nn6)nc5C(=O)O)c4C)(C3)C1)C2)C(=O)OCc1ccc(NC(=O)[C@H](CCCNC(N)=O)NC(=O)[C@@H](NC(=O)CCOCCN2C(=O)C=CC2=O)C(C)C)cc1] |
| L9A-P30 | 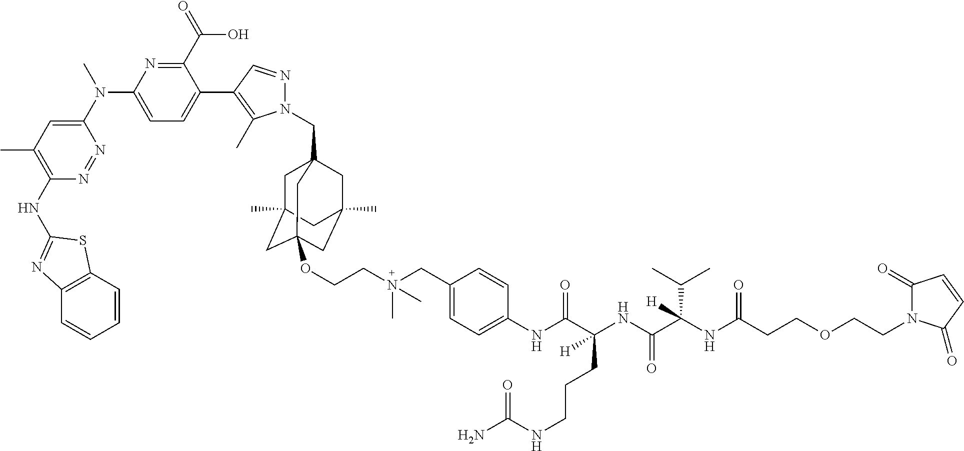 |
| L9C-P31 | 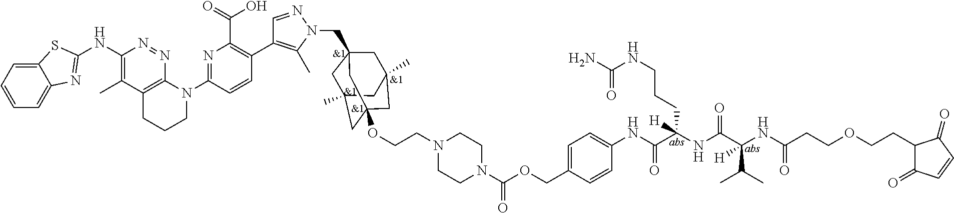 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CCN(C(=O)OCc6ccc(NC(=O)[C@H](CCCNC(N)=O)NC(=O)[C@@H](NC(=O)CCOCCC7C(=O)C=CC7=O)C(C)C)cc6)CC3)(C5)C4)c2C)c(C(=O)O)n1] |
| L9A-P32 | 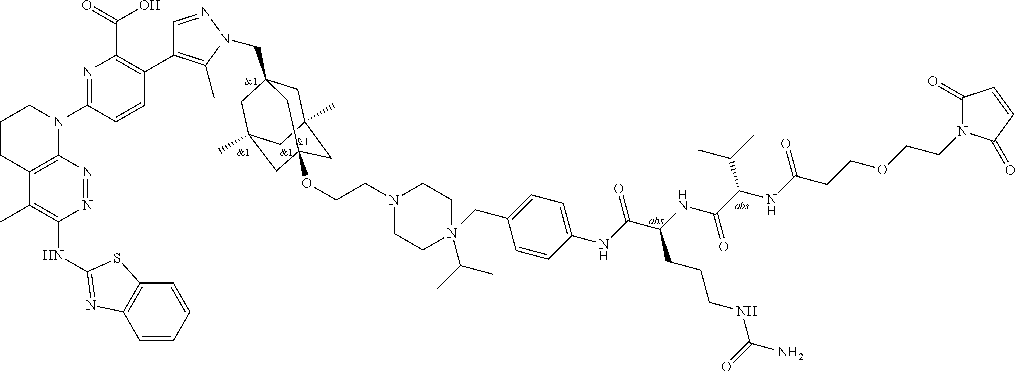 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CC[N+](Cc6ccc(NC(=O)[C@H](CCCNC(N)=O)NC(=O)[C@@H](NC(=O)CCOCCN7C(=O)C=CC7=O)C(C)C)cc6)(C(C)C)CC3)(C5)C4)c2C)c(C(=O)O)n1] |
| L9A-P33 | 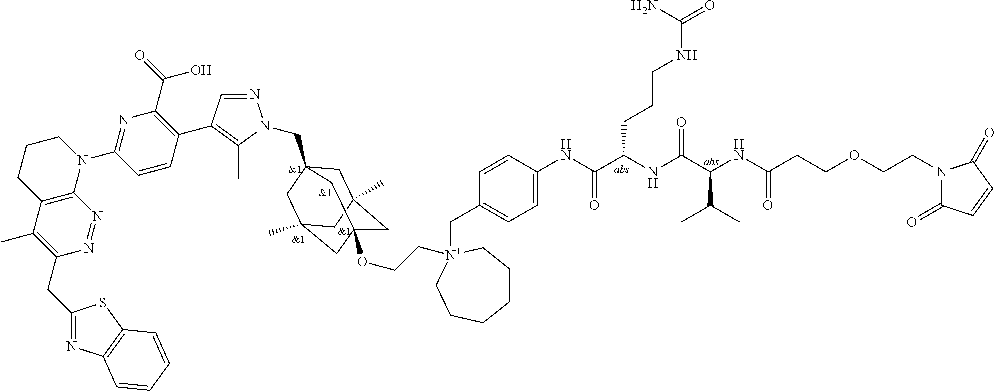 [SMILES: Cc1c(Cc2nc3ccccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCCCC3)(C5)C4)c2C)c(C(=O)O)n1] |
| L9A-P34 | 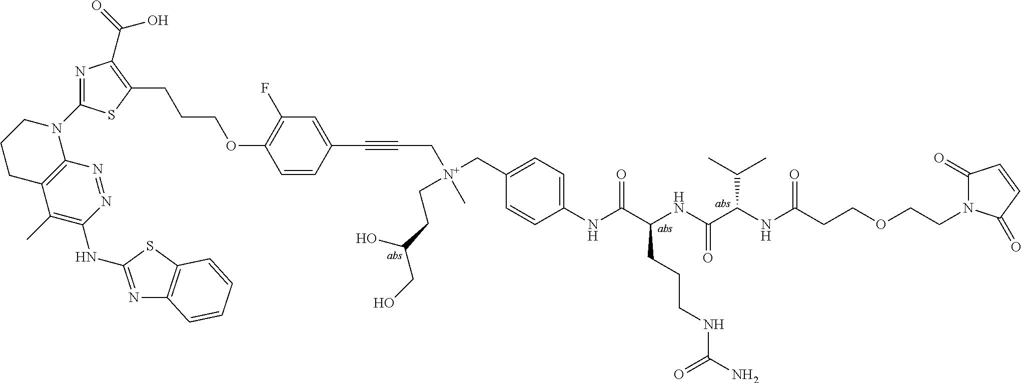 |
| L9A-P35 | 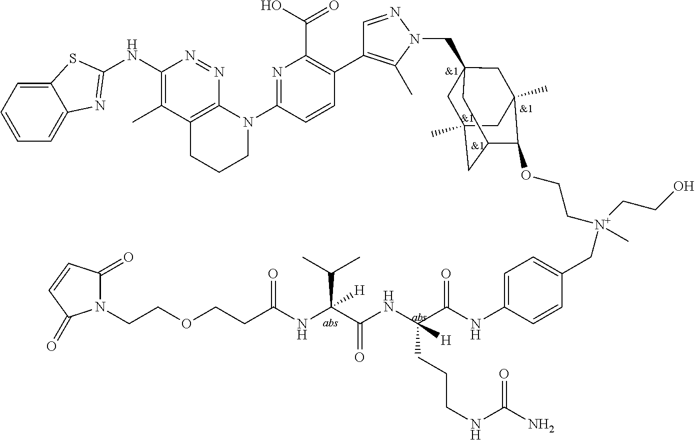 [SMILES: Cc1c(Nc2nc3ccccc3s2)nnc2c1CCCN2c1ccc(-c2cnn(C[C@@]34CC5C[C@@](C)(C3)C[C@@](C)(C4)[C@H]5OCC[N+](C)(CCO)Cc3ccc(NC(=O)[C@H](CCCNC(N)=O)NC(=O)[C@@H](NC(=O)CCOCCN4C(=O)C=CC4=O)C(C)C)cc3)c2C)c(C(=O)O)n1] |
| L9A-P36 | 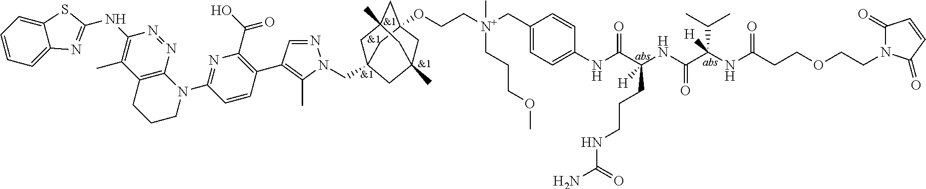 [SMILES: COCCC[N+](C)(CCO[C@]12C[C@@]3(C)C[C@](C)(C[C@](Cn4ncc(-c5ccc(N6CCCc7c6nnc(Nc6nc8ccccc8s6)c7C)nc5C(=O)O)c4C)(C3)C1)C2)Cc1ccc(NC(=O)[C@H](CCCNC(N)=O)NC(=O)[C@@H](NC(=O)CCOCCN2C(=O)C=CC2=O)C(C)C)cc1] |
| L9A-P37 | 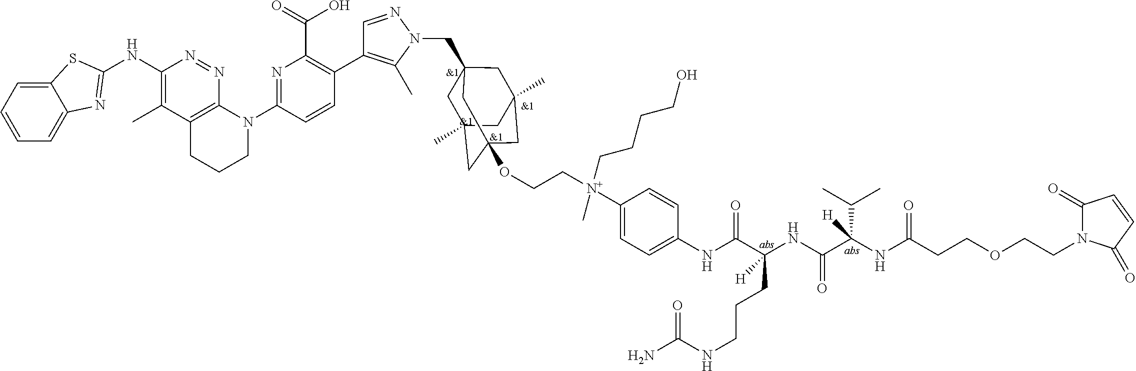 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CCCCO)c3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P38 | 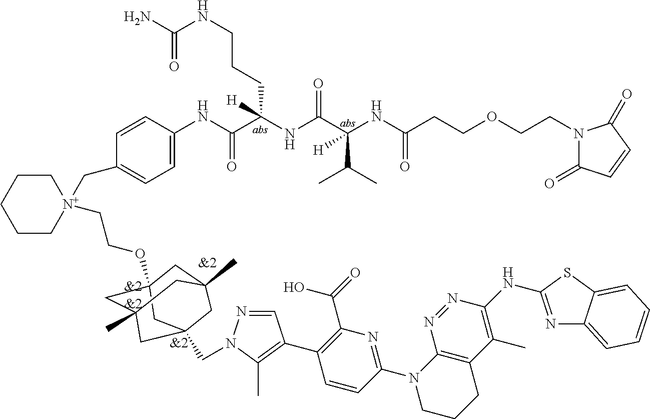 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCCC3)(C5)C4)c2C)c(C(=O)O)n1] |
| L9A-P39 | 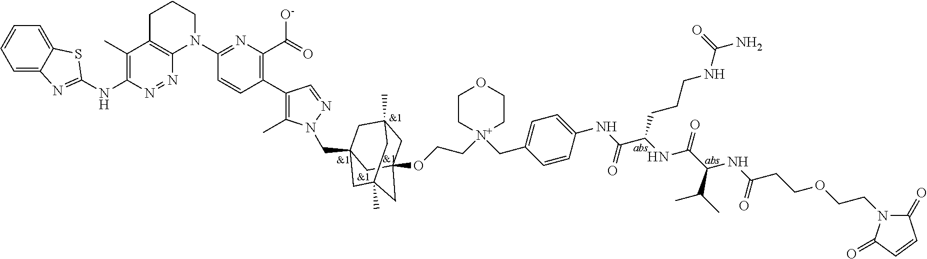 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCOCC3)(C5)C4)c2C)c(C(=O)[O-])n1] |
| L9C-P40 | 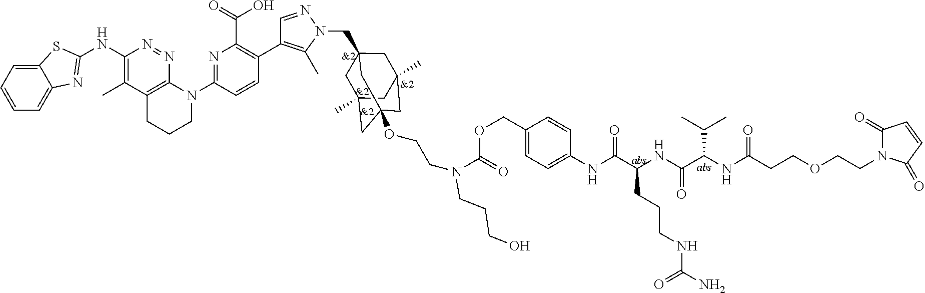 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P41 | 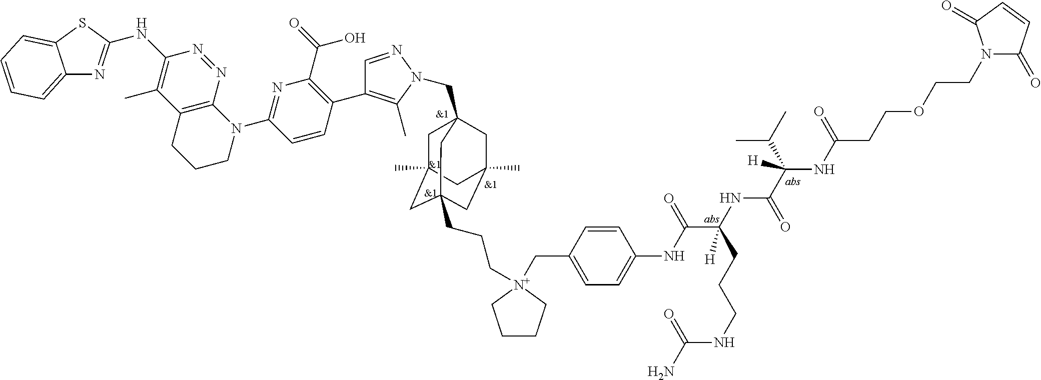 [SMILES: Cc1c(Nc2nc3ccccc3s2)nnc2c1CCCN2c1ccc(-c2cnn(C[C@]34C[C@@]5(C)C[C@](C)(C[C@](CCC[N+]6(Cc7ccc(NC(=O)[C@H](CCCNC(N)=O)NC(=O)[C@@H](NC(=O)CCOCCN8C(=O)C=CC8=O)C(C)C)cc7)CCCC6)(C5)C3)C4)c2C)c(C(=O)O)n1] |
| L9A-P42 | 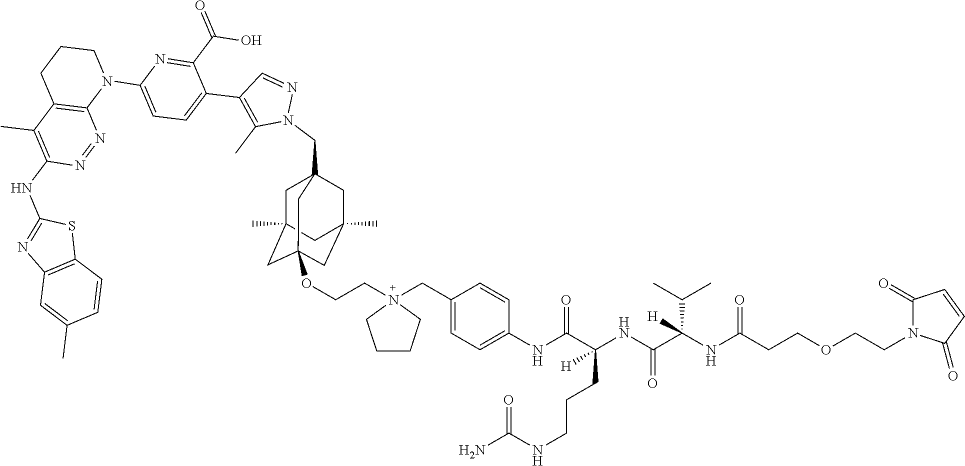 |
| L9A-P43 | 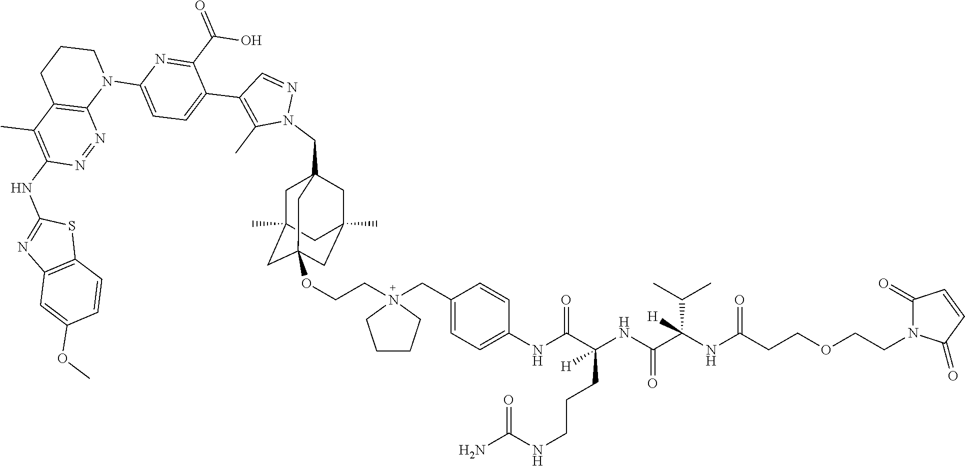 |
| L9C-P44 | 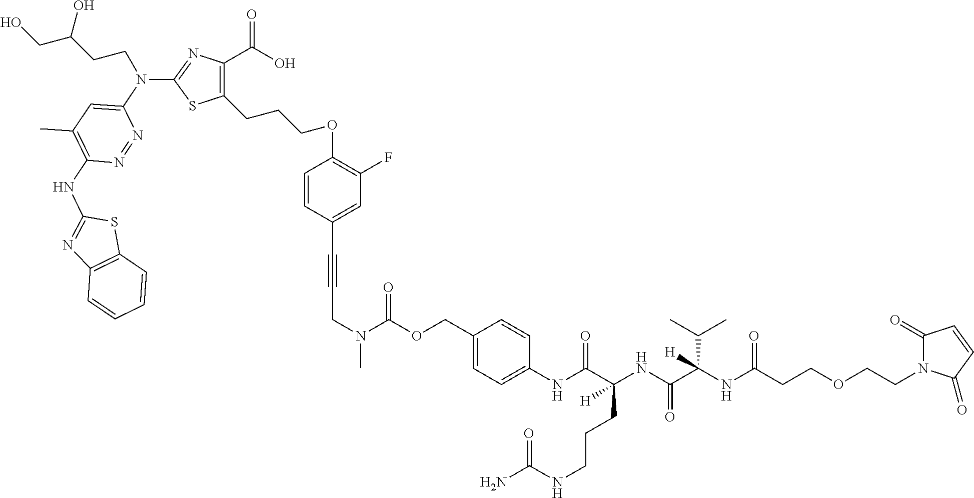 [SMILES: Cc1cc(N(CCC(O)CO)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P45 | 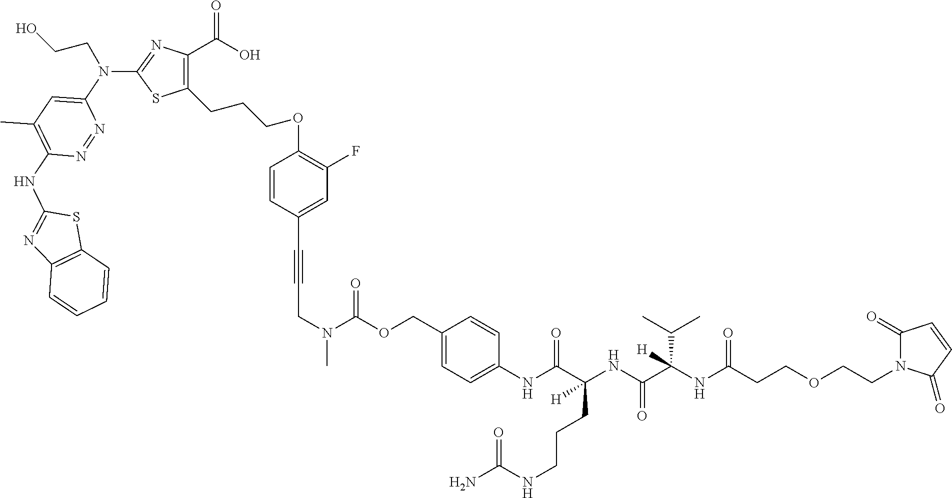 [SMILES: Cc1cc(N(CCO)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P46 | 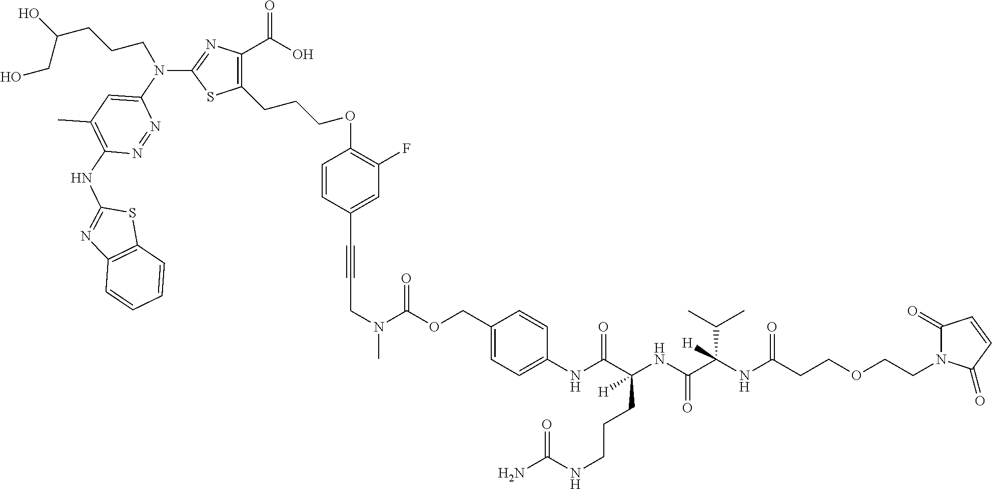 [SMILES: Cc1cc(N(CCCC(O)CO)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P4 | 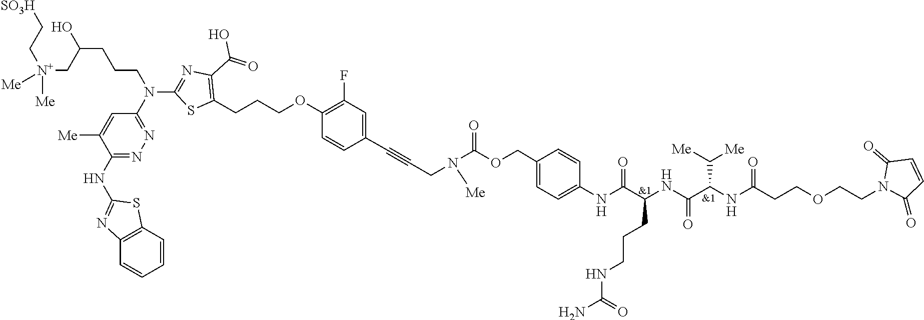 [SMILES: Cc1cc(N(CCCC(O)C[N+](C)(C)CCS(=O)(=O)O)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P5 | 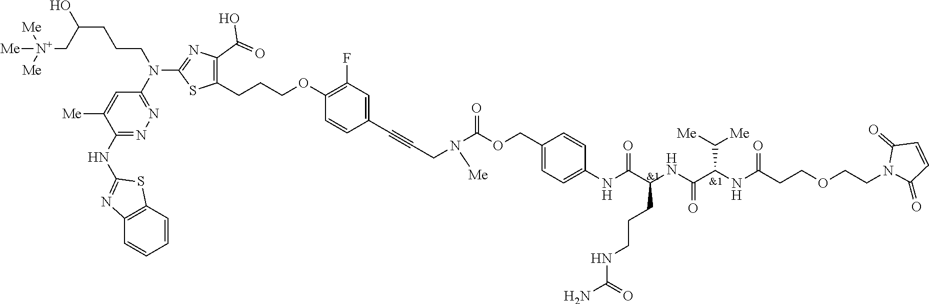 [SMILES: Cc1cc(N(CCCC(O)C[N+](C)(C)C)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P59 | 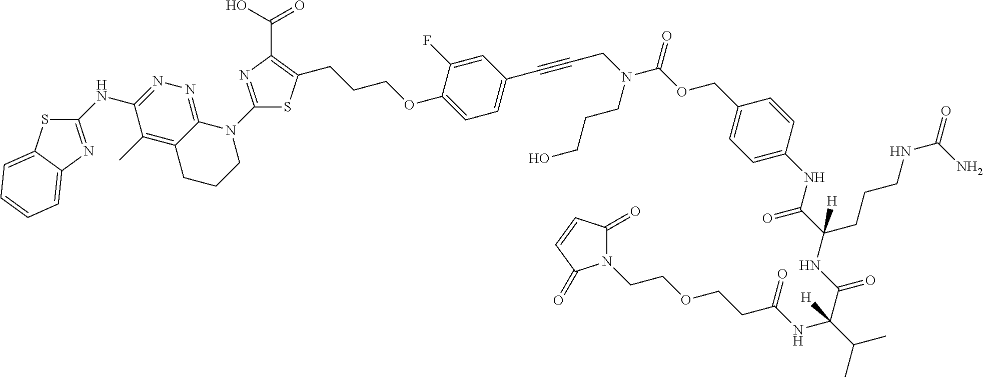 [SMILES: Cc1c(Nc2nc3ccccc3s2)nnc2c1CCCN2c1nc(C(=O)O)c(CCCOc2ccc(C#CCN(CCCO)C(=O)OCc3ccc(NC(=O)[C@H](CCCNC(N)=O)NC(=O)[C@@H](NC(=O)CCOCCN4C(=O)C=CC4=O)C(C)C)cc3)cc2F)s1] |
| L9C-P3 | 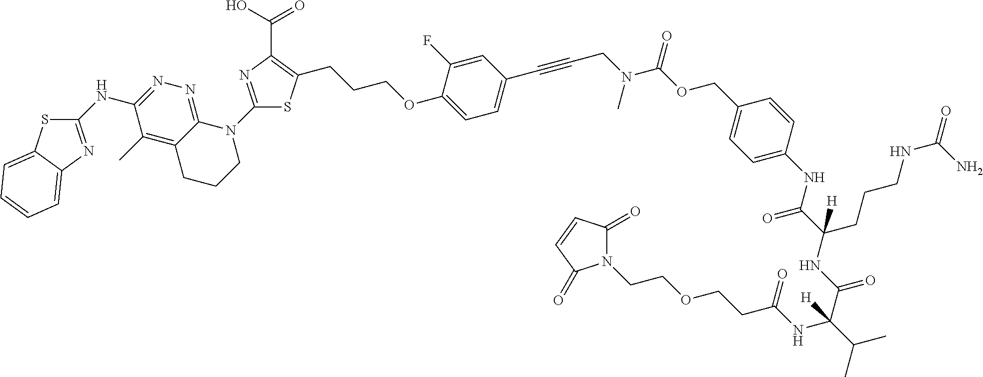 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3)cc2F)s1] |
| L9C-P60 | 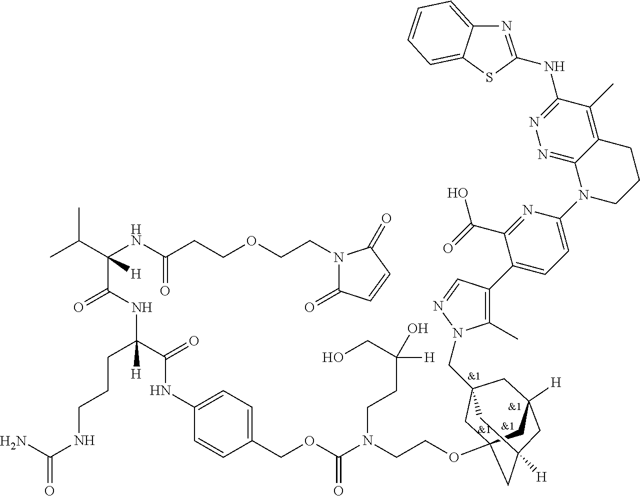 [SMILES: Cc1c(Nc2nc3ccccc3s2)nnc2c1CCCN2c1ccc(-c2cnn(C[C@@]34C[C@H]5C[C@@H](C3)C[C@](OCCN(CCC(O)CO)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P61 | 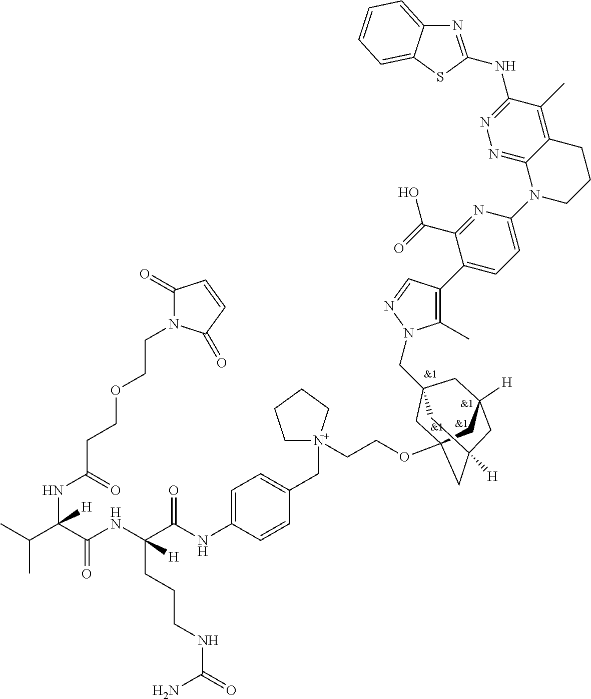 [SMILES: Cc1c(Nc2nc3ccccc3s2)nnc2c1CCCN2c1ccc(-c2cnn(C[C@@]34C[C@H]5C[C@@H](C3)C[C@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(C(=O)O)n1] |
| L9A-P62 | 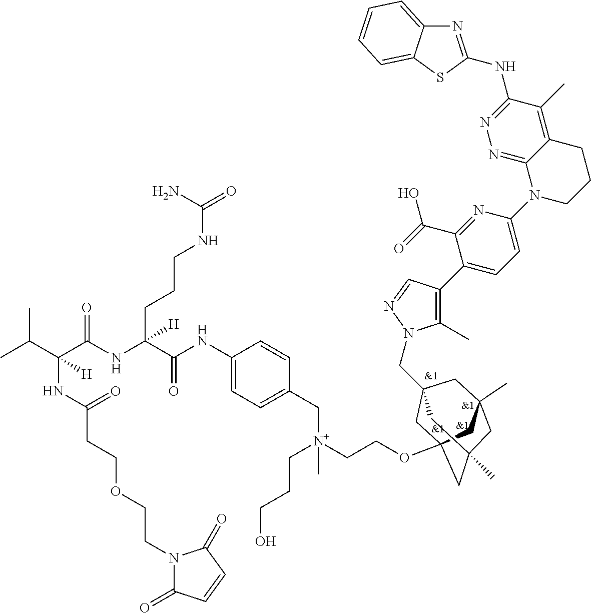 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CCCO)Cc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P63 | 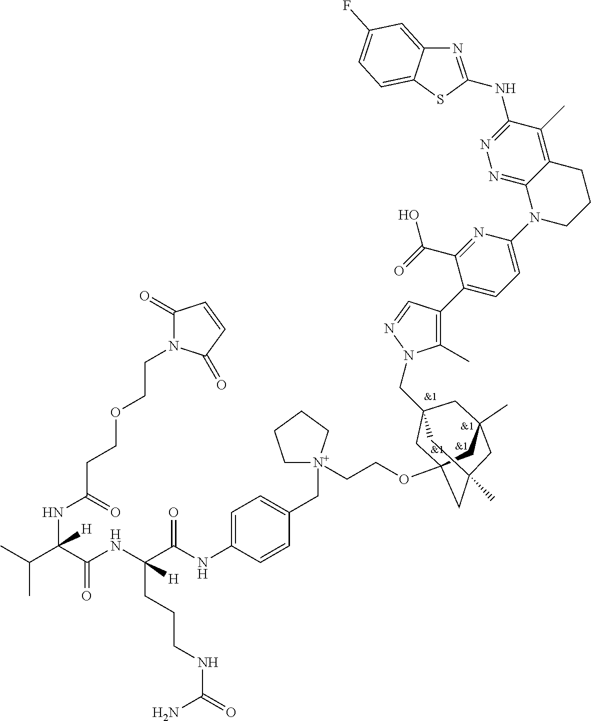 [SMILES: Cc1c(Nc2nc3cc(F)ccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(C(=O)O)n1] |
| L9A-P64 | 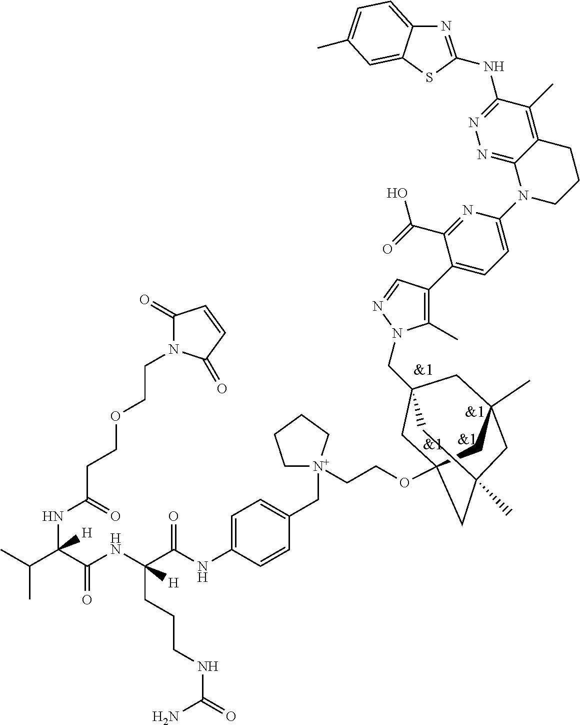 [SMILES: Cc1ccc2nc(Nc3nnc4c(c3C)CCCN4c3ccc(-c4cnn(C[C@]56C[C@]7(C)C[C@](C)(C5)C[C@@](OCC[N+]5(Cc8ccc(NC(=O)[C@H](CCCNC(N)=O)NC(=O)[C@@H](NC(=O)CCOCCN9C(=O)C=CC9=O)C(C)C)cc8)CCCC5)(C7)C6)c4C)c(C(=O)O)n3)sc2c1] |
| L9A-P65 | 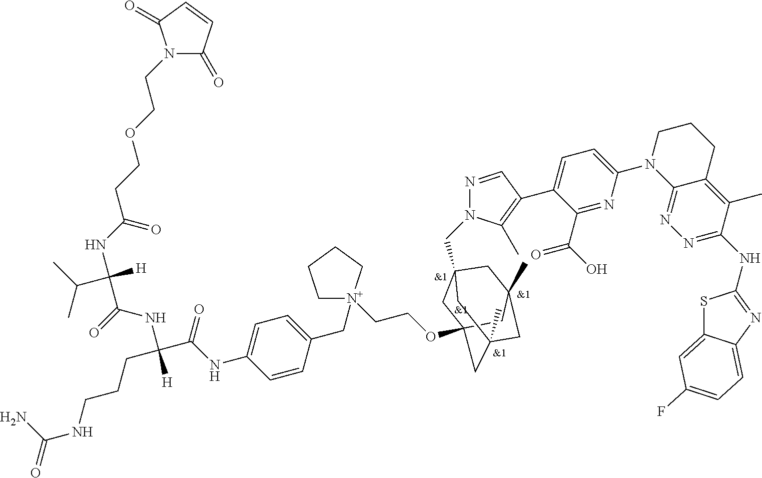 [SMILES: Cc1c(Nc2nc3ccc(F)cc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(C(=O)O)n1] |
| L9A-P66 | 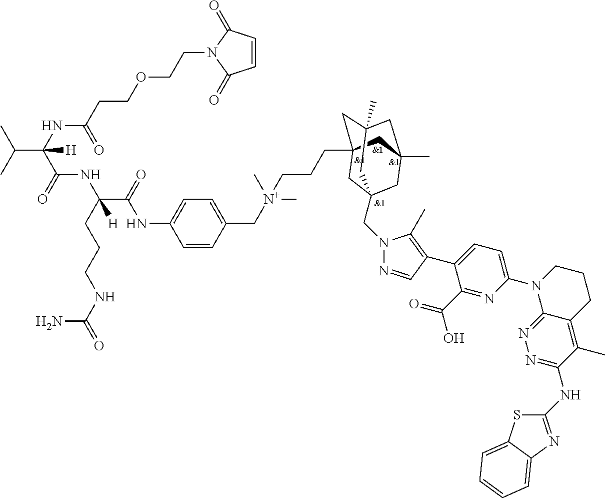 [SMILES: Cc1c(Nc2nc3ccccc3s2)nnc2c1CCCN2c1ccc(-c2cnn(C[C@]34C[C@@]5(C)C[C@](C)(C[C@](CCC[N+](C)(C)Cc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9A-P67 | 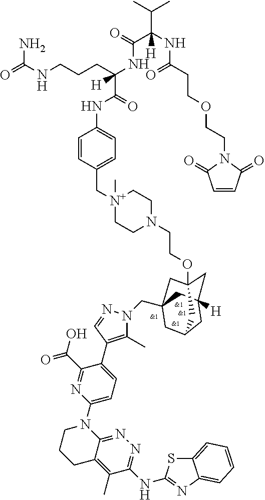 [SMILES: Cc1c(Nc2nc3ccccc3s2)nnc2c1CCCN2c1ccc(-c2cnn(C[C@@]34C[C@H]5C[C@@H](C3)C[C@](OCCN3CC[N+](C)(Cc6ccc(NC(=O)[C@H](CCCNC(N)=O)NC(=O)[C@@H](NC(=O)CCOCCN7C(=O)C=CC7=O)C(C)C)cc6)CC3)(C5)C4)c2C)c(C(=O)O)n1] |
| L9A-P68 | 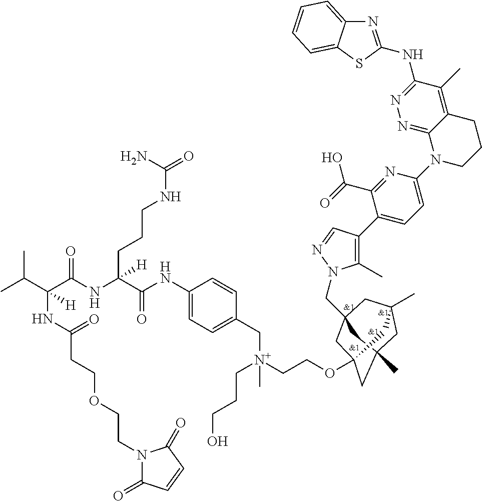 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CCCO)Cc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P69 | 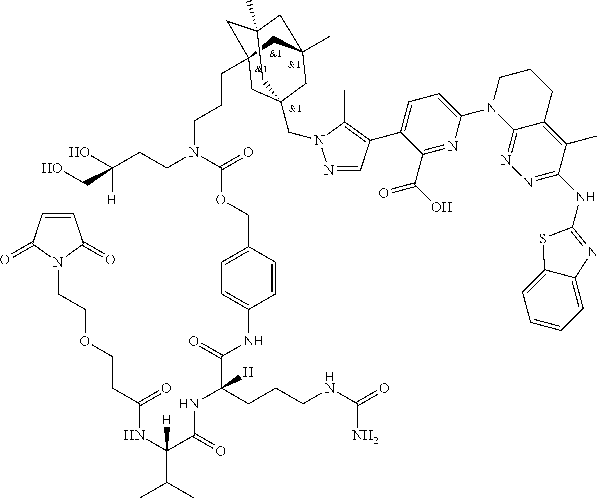 [SMILES: Cc1c(Nc2nc3ccccc3s2)nnc2c1CCCN2c1ccc(-c2cnn(C[C@]34C[C@@]5(C)C[C@](C)(C[C@](CCCN(CC[C@H](O)CO)C(=O)OCc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9A-P48 | 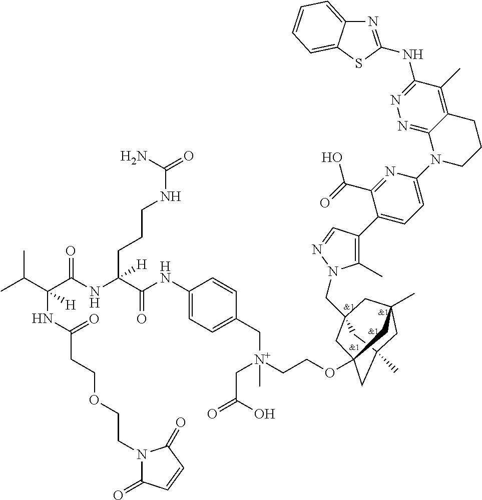 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CC(=O)O)Cc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P70 | 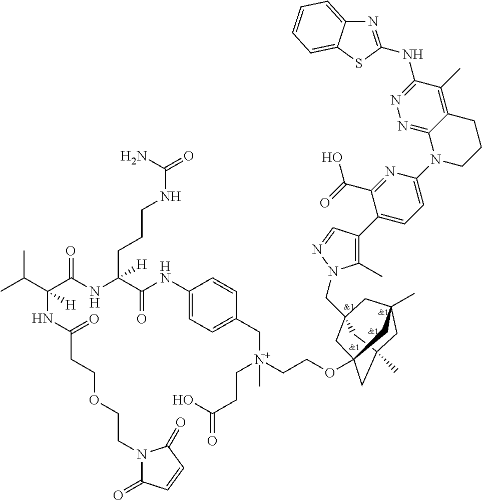 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CCC(=O)O)Cc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P71 | 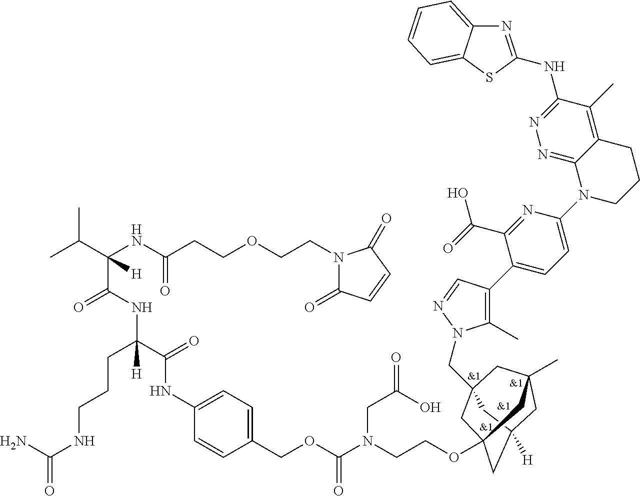 [SMILES: Cc1c(Nc2nc3ccccc3s2)nnc2c1CCCN2c1ccc(-c2cnn(C[C@@]34C[C@H]5C[C@@](C)(C3)C[C@](OCCN(CC(=O)O)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P72 | 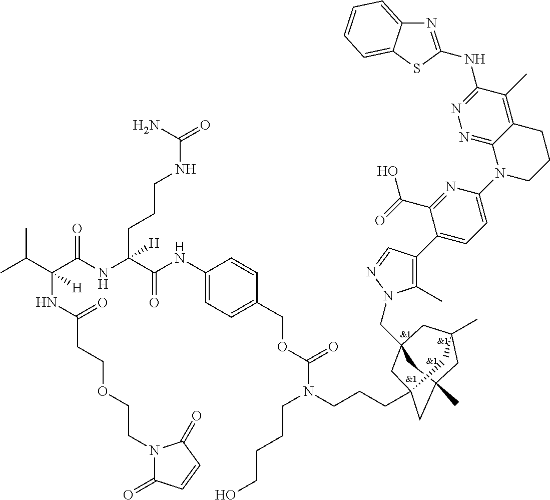 [SMILES: Cc1c(Nc2nc3ccccc3s2)nnc2c1CCCN2c1ccc(-c2cnn(C[C@]34C[C@@]5(C)C[C@](C)(C[C@](CCCN(CCCCO)C(=O)OCc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9A-P49 | 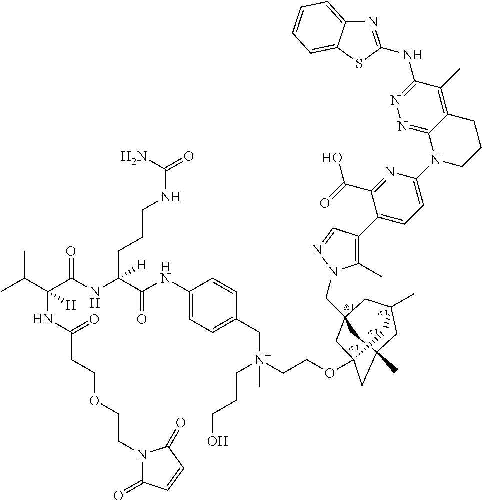 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CCCO)Cc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P51 | 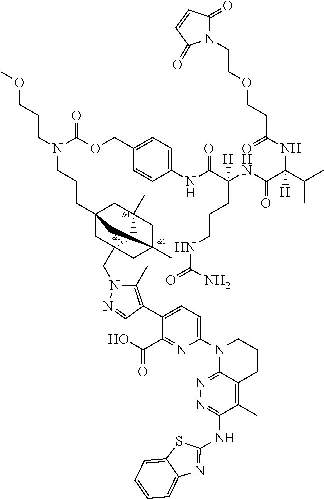 [SMILES: COCCCN(CCC[C@]12C[C@]3(C)C[C@](C)(C1)C[C@@](Cn1ncc(-c4ccc(N5CCCc6c5nnc(Nc5nc7ccccc7s5)c6C)nc4C(=O)O)c1C)(C3)C2)C(=O)OCc1ccc(NC(=O)[C@H](CCCNC(N)=O)NC(=O)[C@@H](NC(=O)CCOCCN2C(=O)C=CC2=O)C(C)C)cc1] |
| L9A-P50 | 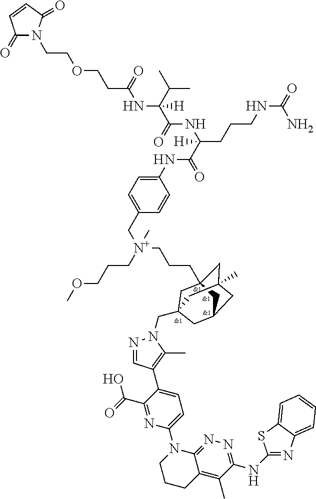 [SMILES: COCCC[N+](C)(CCC[C@]12C[C@@H]3C[C@](C)(C1)C[C@@](Cn1ncc(-c4ccc(N5CCCc6c5nnc(Nc5nc7ccccc7s5)c6C)nc4C(=O)O)c1C)(C3)C2)Cc1ccc(NC(=O)[C@H](CCCNC(N)=O)NC(=O)[C@@H](NC(=O)CCOCCN2C(=O)C=CC2=O)C(C)C)cc1] |
| L9A-P52 | 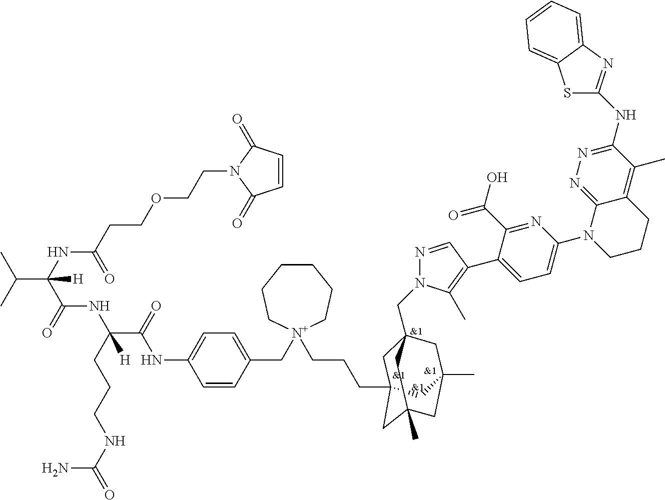 [SMILES: Cc1c(Nc2nc3ccccc3s2)nnc2c1CCCN2c1ccc(-c2cnn(C[C@]34C[C@@]5(C)C[C@](C)(C[C@](CCC[N+]6(Cc7ccc(NC(=O)[C@H](CCCNC(N)=O)NC(=O)[C@@H](NC(=O)CCOCCN8C(=O)C=CC8=O)C(C)C)cc7)CCCCCC6)(C5)C3)C4)c2C)c(C(=O)O)n1] |
| L9C-P53 | 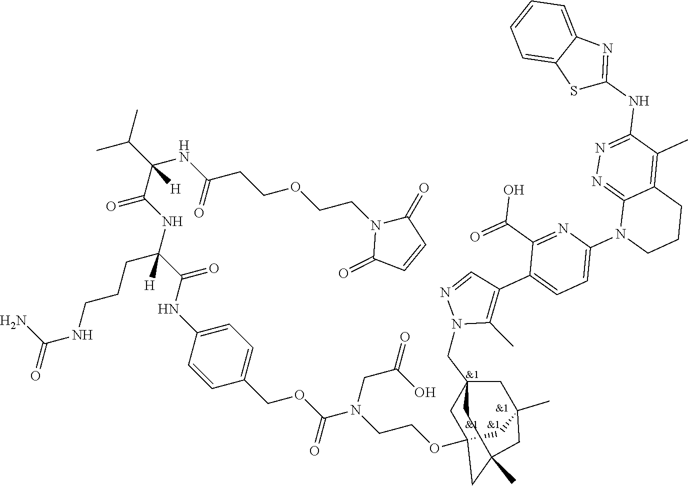 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CC(=O)O)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P55 | 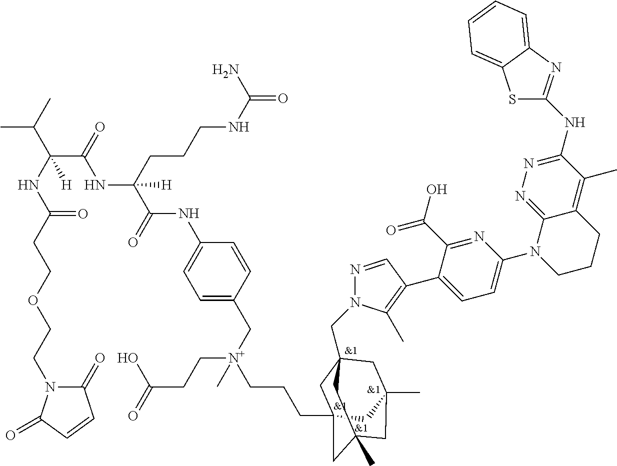 [SMILES: Cc1c(Nc2nc3ccccc3s2)nnc2c1CCCN2c1ccc(-c2cnn(C[C@]34C[C@@]5(C)C[C@](C)(C[C@](CCC[N+](C)(CCC(=O)O)Cc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9C-P54 | 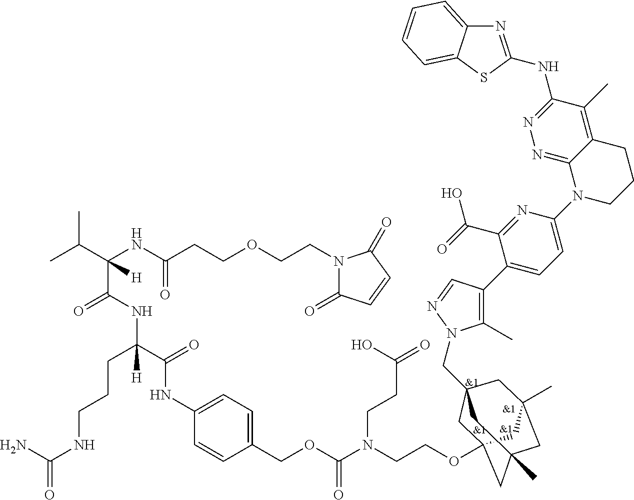 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CCC(=O)O)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P47 | 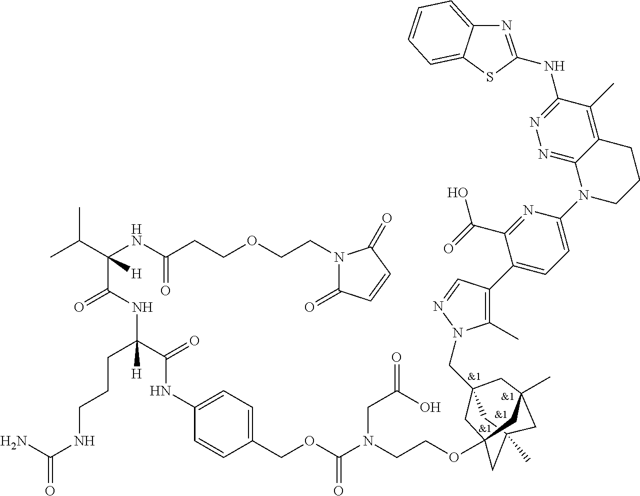 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CC(=O)O)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P56 | 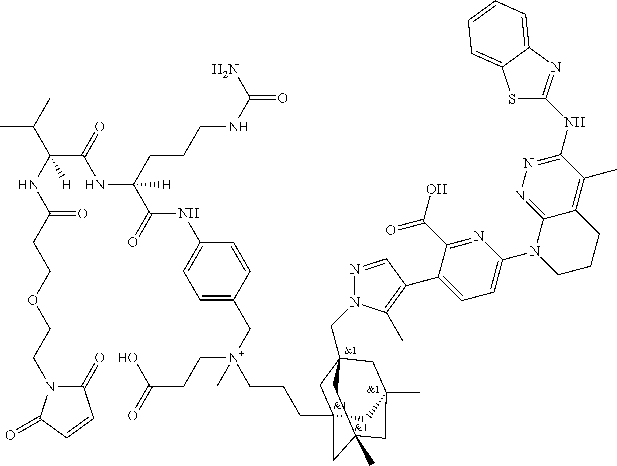 [SMILES: Cc1c(Nc2nc3ccccc3s2)nnc2c1CCCN2c1ccc(-c2cnn(C[C@]34C[C@@]5(C)C[C@](C)(C[C@](CCC[N+](C)(CCC(=O)O)Cc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9A-P58 | 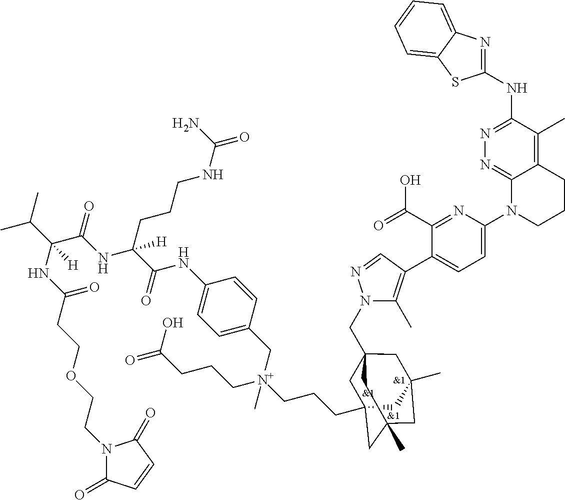 [SMILES: Cc1c(Nc2nc3ccccc3s2)nnc2c1CCCN2c1ccc(-c2cnn(C[C@]34C[C@@]5(C)C[C@](C)(C[C@](CCC[N+](C)(CCCC(=O)O)Cc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9A-P57 | 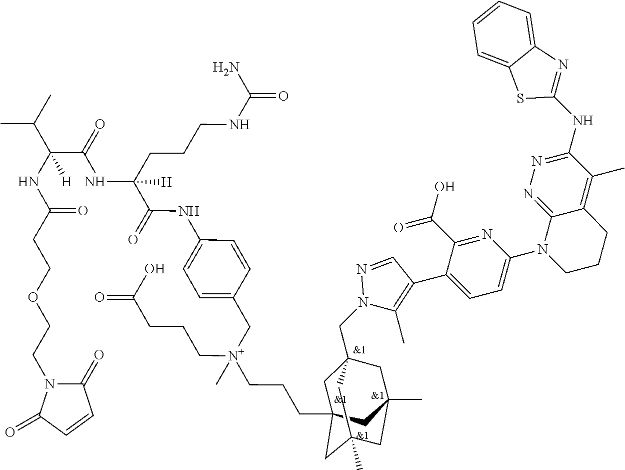 [SMILES: Cc1c(Nc2nc3ccccc3s2)nnc2c1CCCN2c1ccc(-c2cnn(C[C@]34C[C@@]5(C)C[C@](C)(C[C@](CCC[N+](C)(CCCC(=O)O)Cc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9A-P73 | 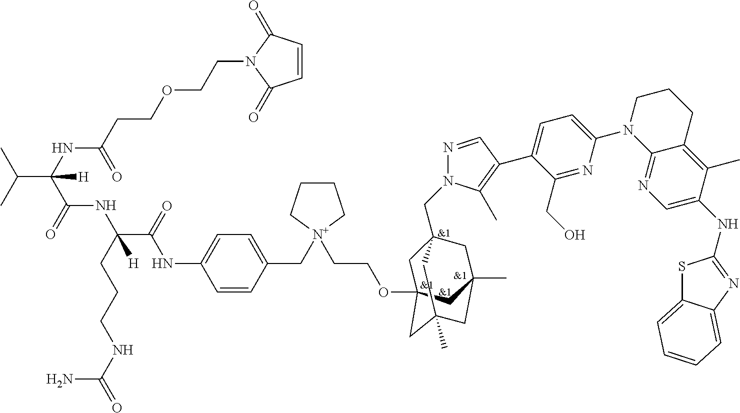 [SMILES: Cc1c(Nc2nc3ccccc3s2)cnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(CO)n1] |
| L9A-P74 | 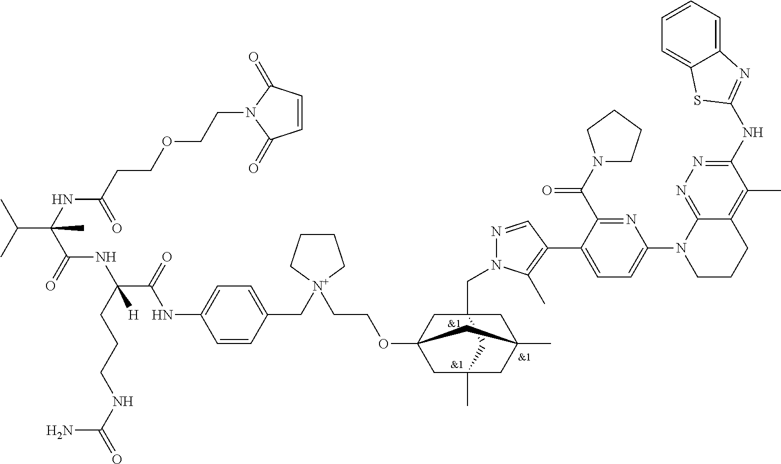 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@](C)(NC(=O)CCOCCN7C(=O)C=CC7=O)C(C)C)cc6)CCCC3)(C5)C4)c2C)c(C(=O)N2CCCC2)n1] |
| L9A-P75 | 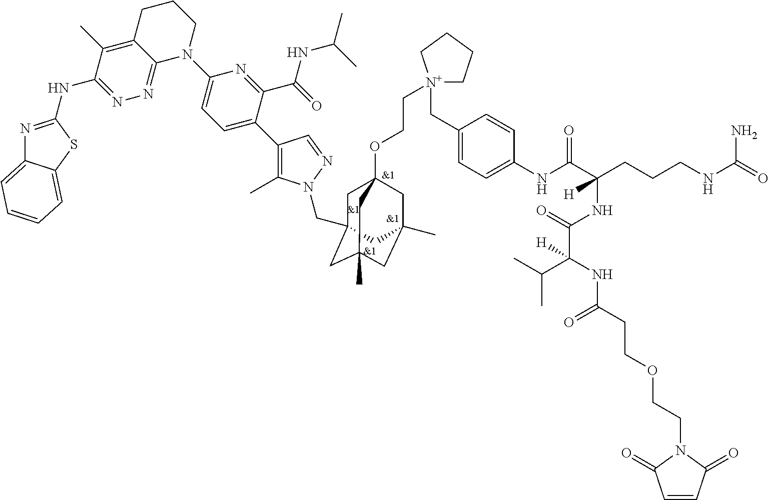 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(C(=O)NC(C)C)n1] |
| L9A-P76 | 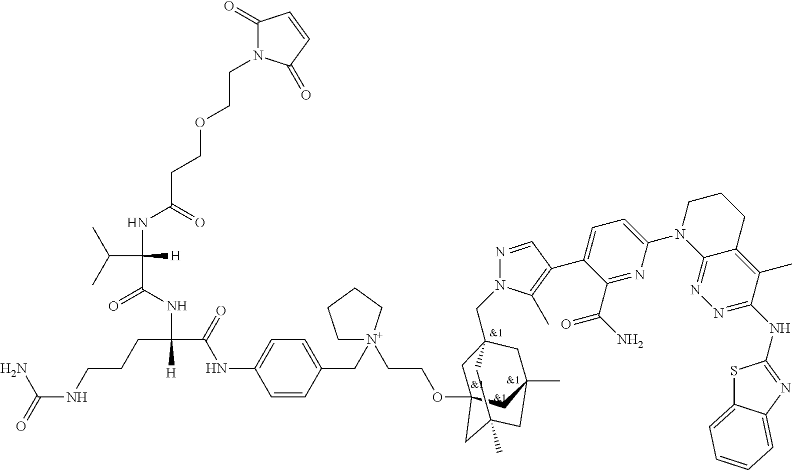 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(C(N)=O)n1] |
| L10A-P1 | 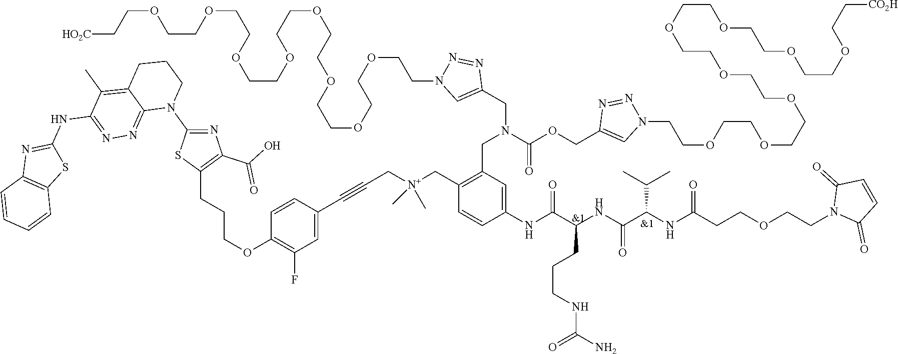 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(Cc3cn(CCOCCOCCOCCOCCOCCOCCOCCOCCC(=O)O)nn3)C(=O)OCc3cn(CCOCCOCCOCCOCCOCCOCCOCCOCCC(=O)O)nn3)cc2F)s1] |
| L10A-P2 | 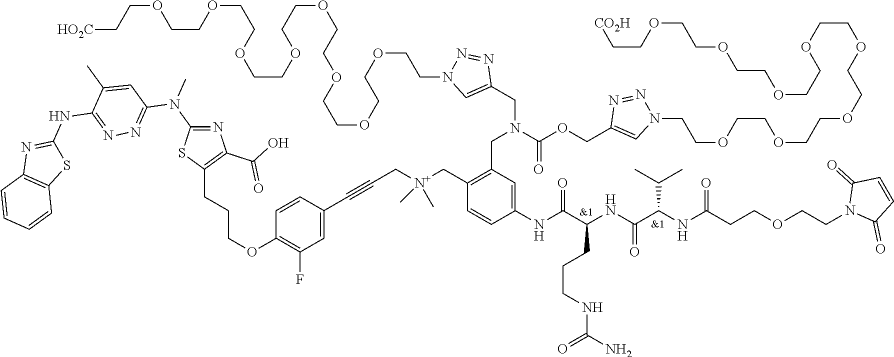 [SMILES: Cc1cc(N(C)c2nc(C(=O)O)c(CCCOc3ccc(C#CC[N+](C)(C)Cc4ccc(NC(=O)[C@H](CCCNC(N)=O)NC(=O)[C@@H](NC(=O)CCOCCN5C(=O)C=CC5=O)C(C)C)cc4CN(Cc4cn(CCOCCOCCOCCOCCOCCOCCOCCOCCC(=O)O)nn4)C(=O)OCc4cn(CCOCCOCCOCCOCCOCCOCCOCCOCCC(=O)O)nn4)cc3F)s2)nnc1Nc1nc2ccccc2s1] |
| L10C-P3 | 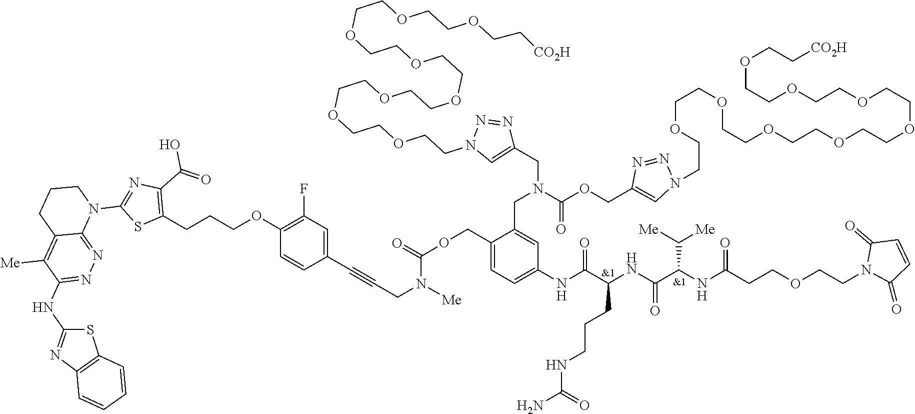 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3CN(Cc3cn(CCOCCOCCOCCOCCOCCOCCOCCOCCC(=O)O)nn3)C(=O)OCc3cn(CCOCCOCCOCCOCCOCCOCCOCCOCCC(=O)O)nn3)cc2F)s1] |
| L11A-P1 | 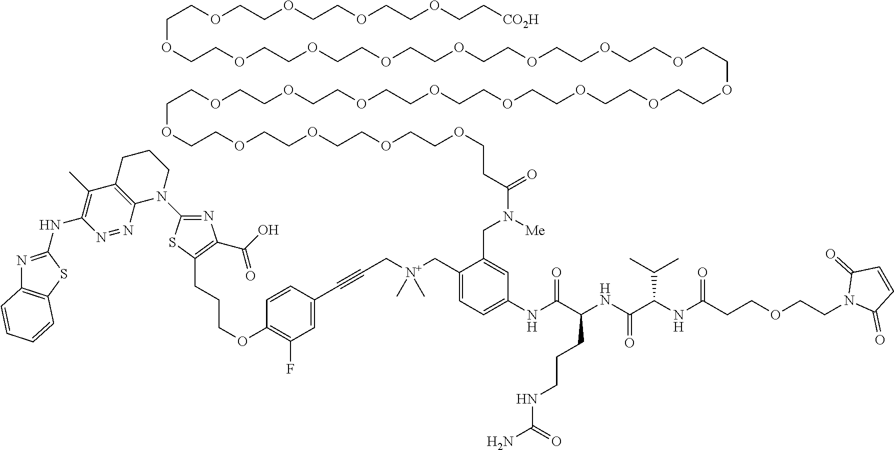 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)cc2F)s1] |
| L11A-P21 | 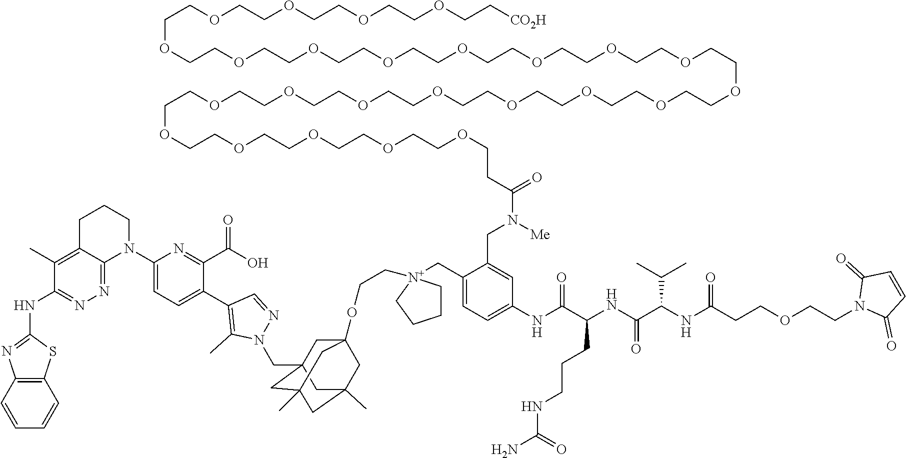 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)CCCC3)(C5)C4)c2C)c(C(=O)O)n1] |
| L11C-P25 | 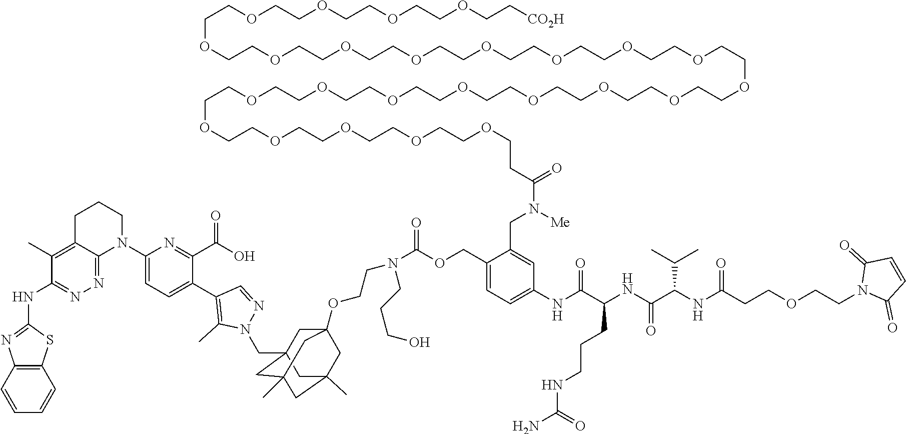 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1] |
| L11A-P27 | 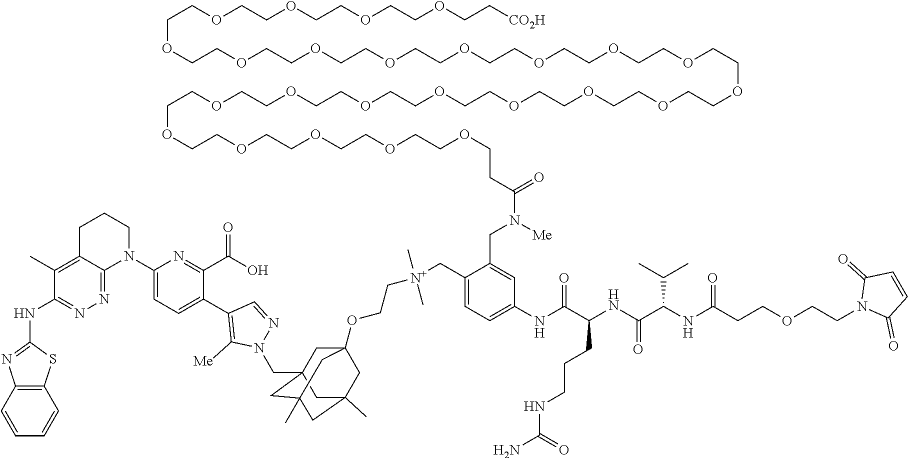 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1] |
| L11C-P19 | 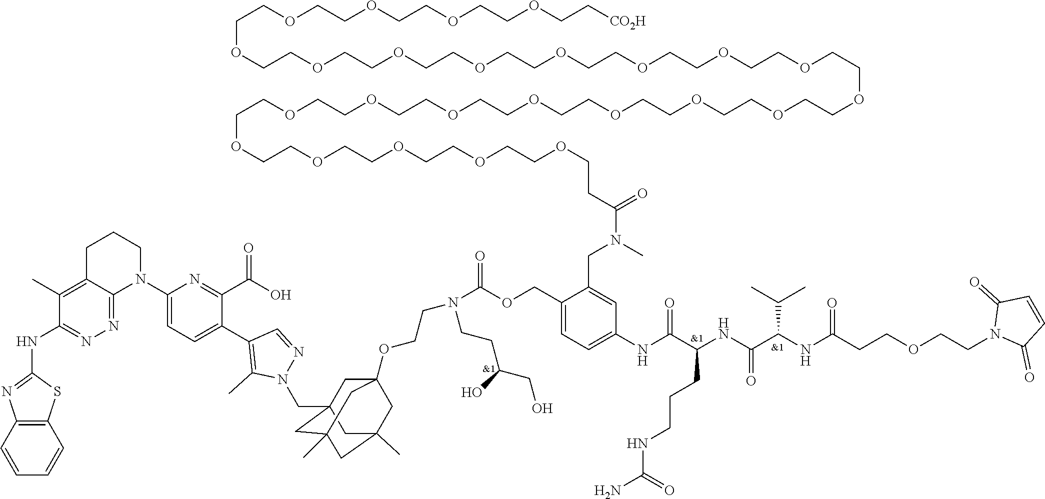 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1] |
| L13A-P2 | 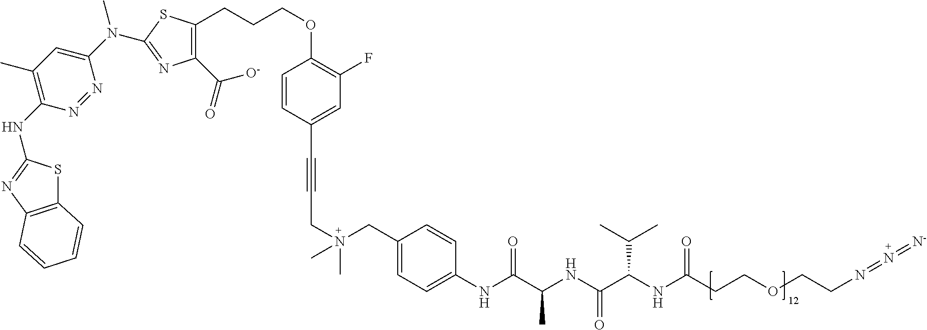 |
| L19C-P7 | 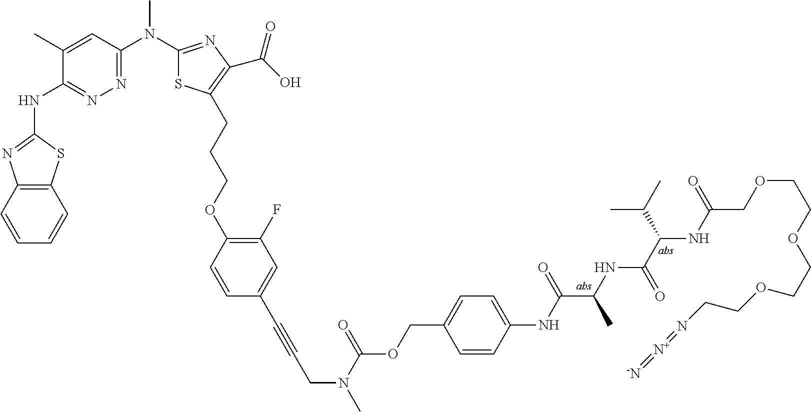 |
| L21A-P2 | 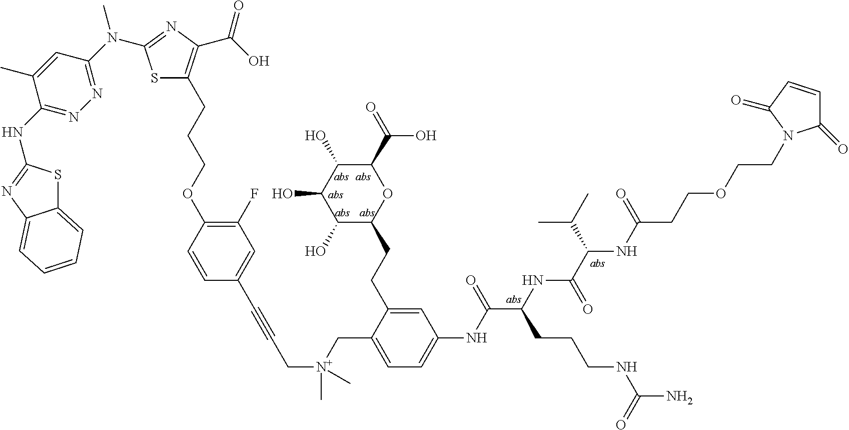 [SMILES: Cc1cc(N(C)c2nc(C(=O)O)c(CCCOc3ccc(C#CC[N+](C)(C)Cc4ccc(NC(=O)[C@H](CCCNC(N)=O)NC(=O)[C@@H](NC(=O)CCOCCN5C(=O)C=CC5=O)C(C)C)cc4CC[C@@H]4O[C@H](C(=O)O)[C@@H](O)[C@H](O)[C@H]4O)cc3F)s2)nnc1Nc1nc2ccccc2s1] |
| L23C-P7 | 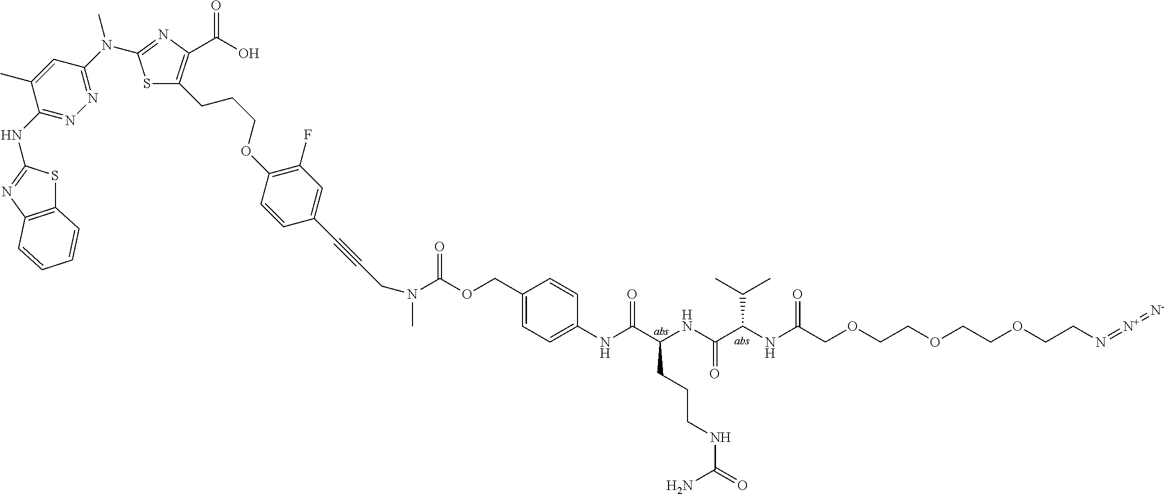 [SMILES: Cc1cc(N(C)c2nc(C(=O)O)c(CCCOc3ccc(C#CCN(C)C(=O)OCc4ccc(NC(=O)[C@H](CCCNC(N)=O)NC(=O)[C@@H](NC(=O)COCCOCCOCCN=[N+]=[N-])C(C)C)cc4)cc3F)s2)nnc1Nc1nc2ccccc2s1] |
| L27C-P3 | 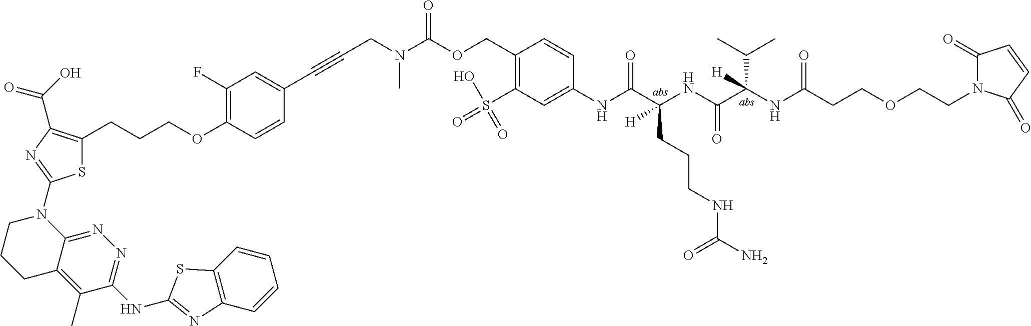 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3S(=O)(=O)O)cc2F)s1] |
| L27A-P1 | 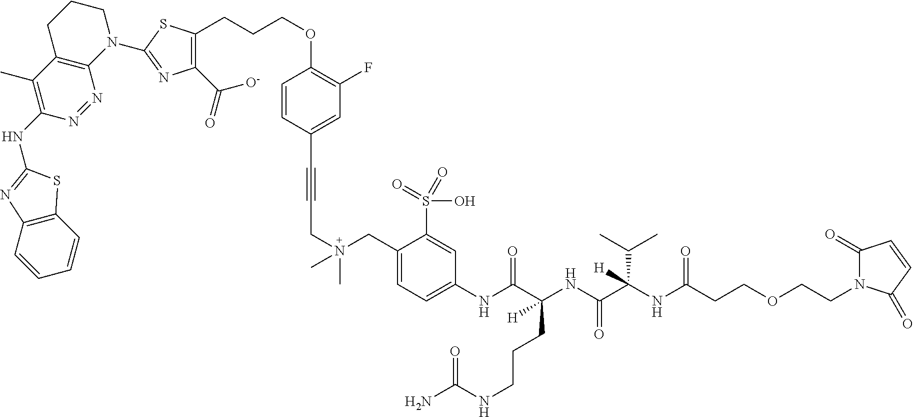 |
| L30A-P1 | 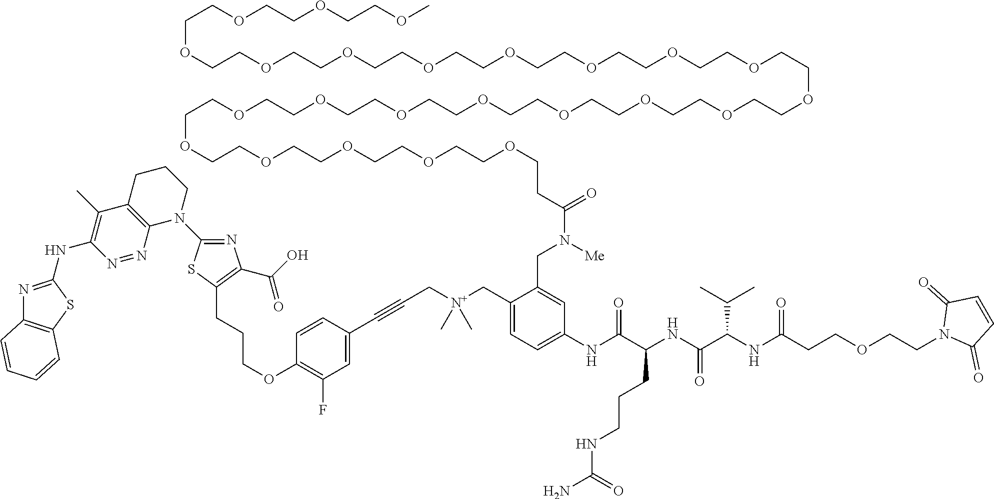 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1] |
| L30C-P19 | 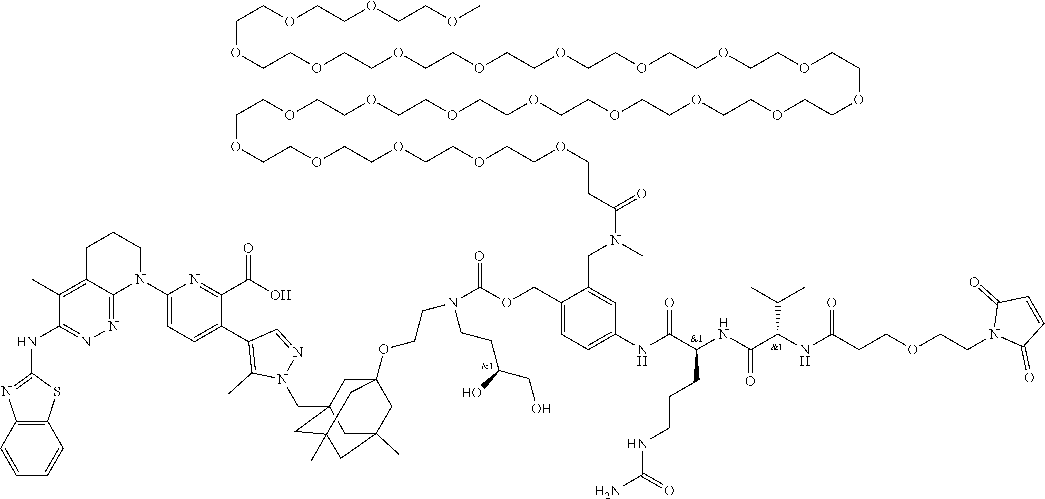 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO] |
| L30A-P21 | 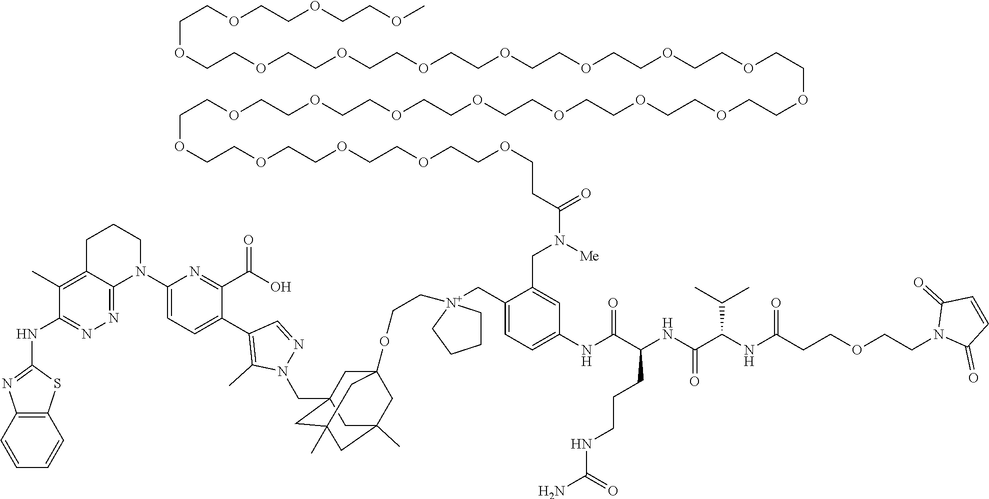 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1] |
| L30C-P25 | 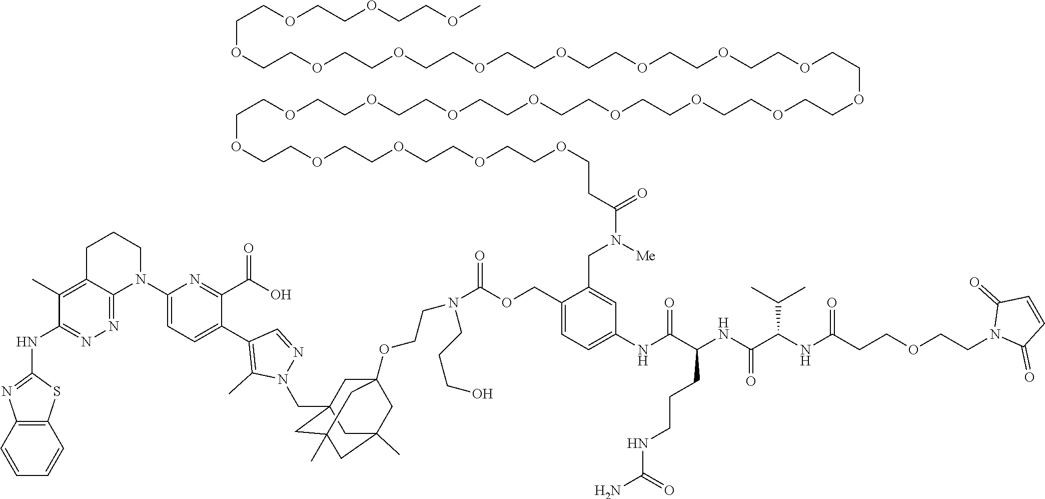 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCCO)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L30A-P27 | 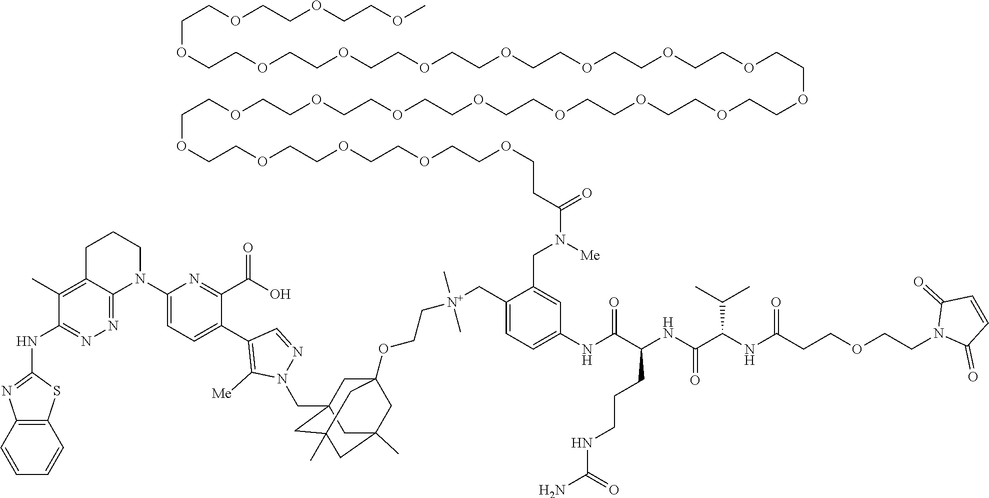 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L35A-P1 | 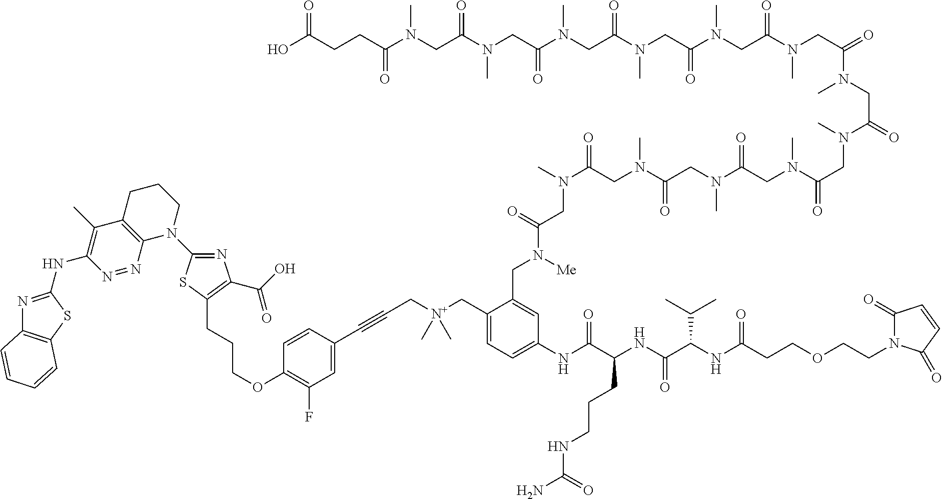 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)cc2F)s1] |
| L35C-P19 | 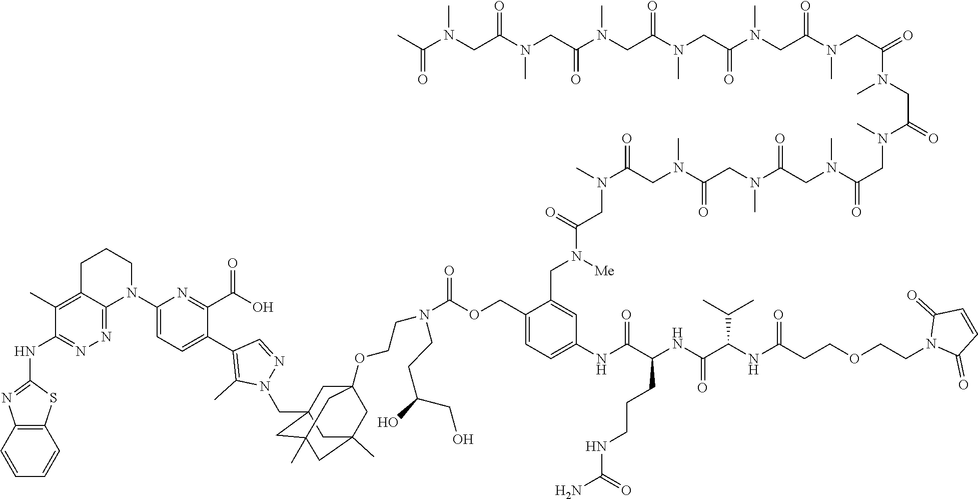 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO] |
| L35A-P21 | 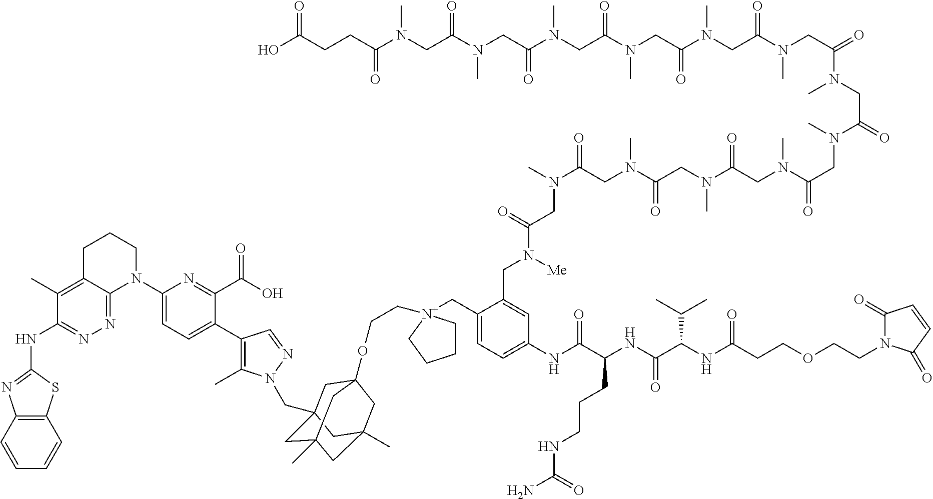 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)CCCC3)(C5)C4)c2C)c(C(=O)O)n1] |
| L35C-P25 | 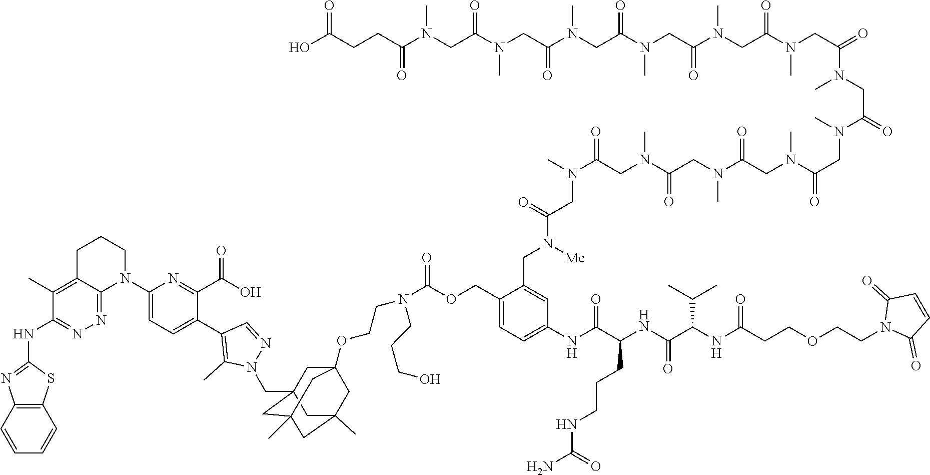 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L35A-P27 | 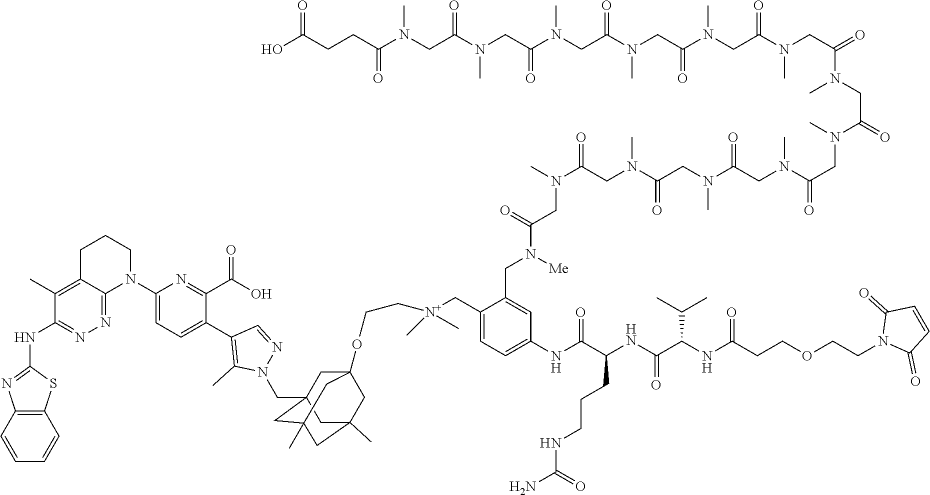 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L36A-P1 | 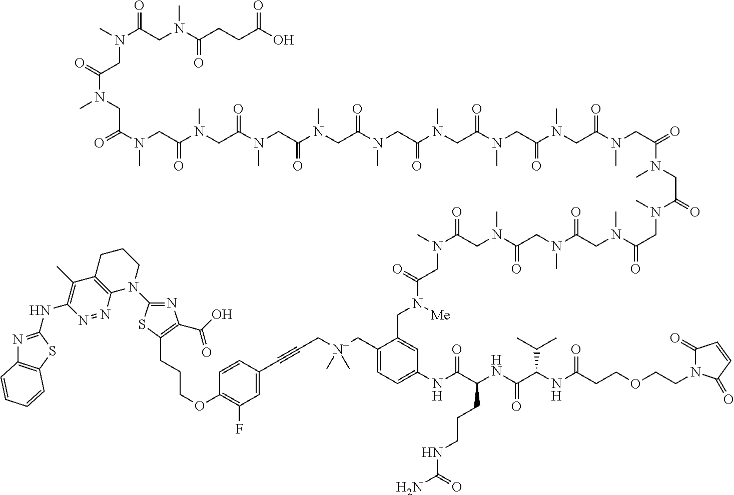 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)cc2F)s1] |
| L36C-P19 | 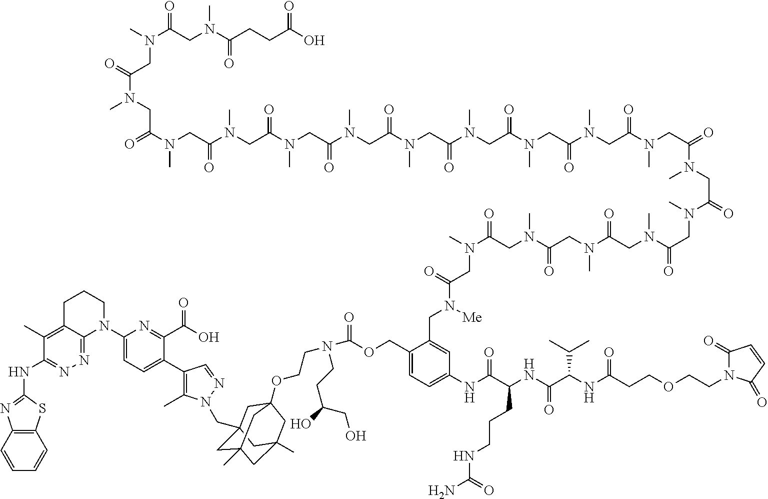 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L36A-P21 | 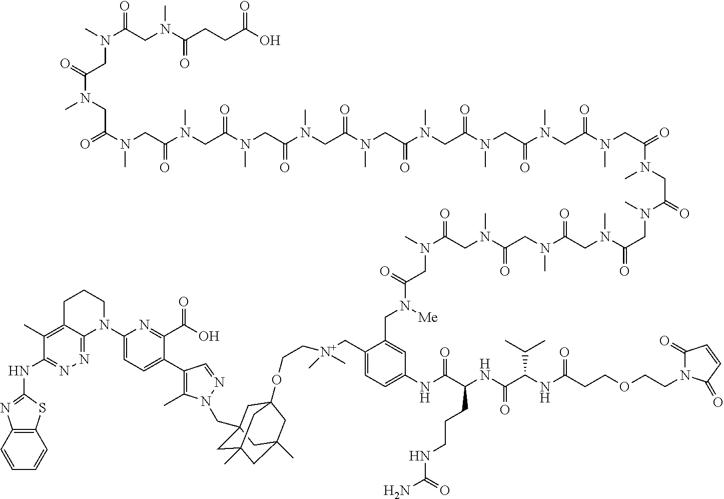 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L36C-P25 | 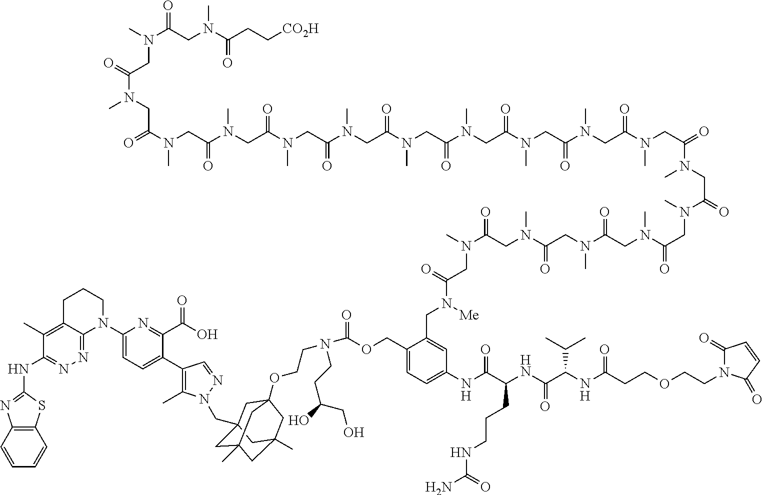 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L36A-P27 | 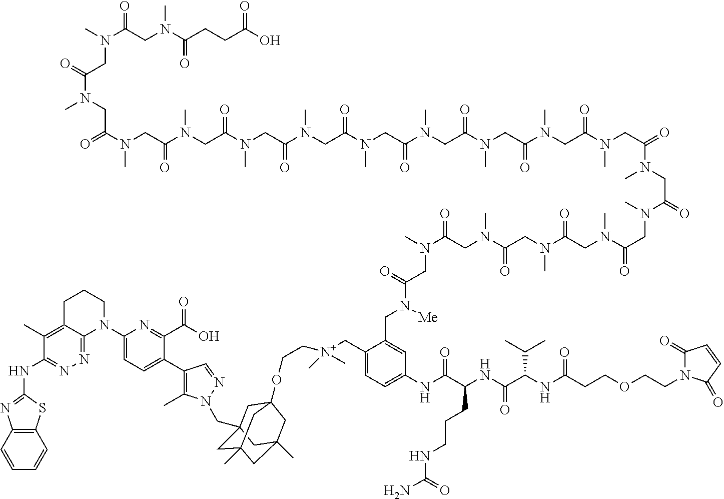 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L37A-P1 | 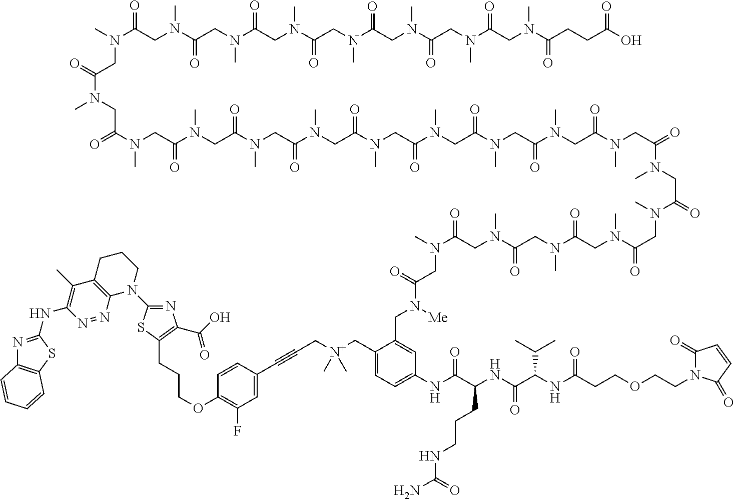 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)cc2F)s1] |
| L37C-P19 | 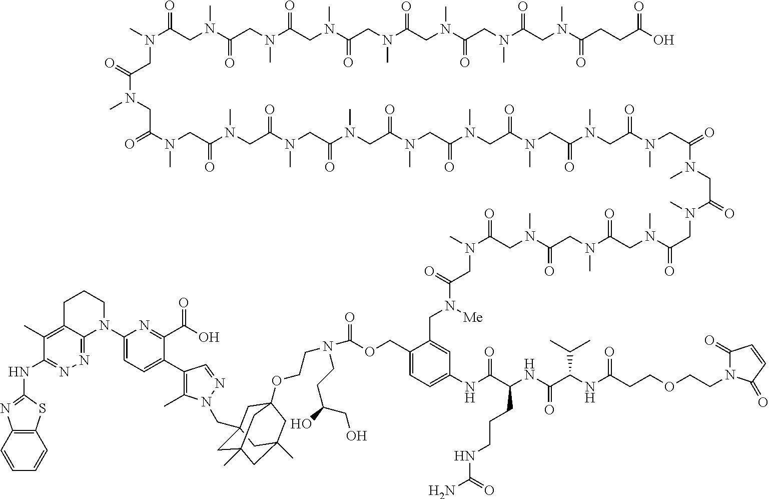 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L37A-P21 | 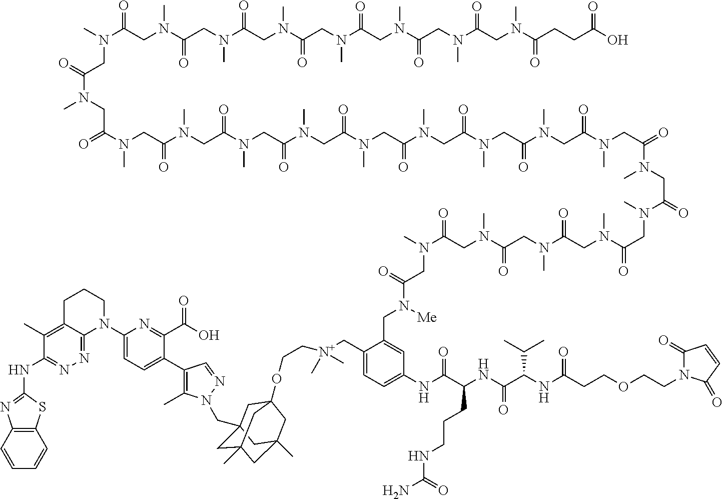 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L37C-P25 | 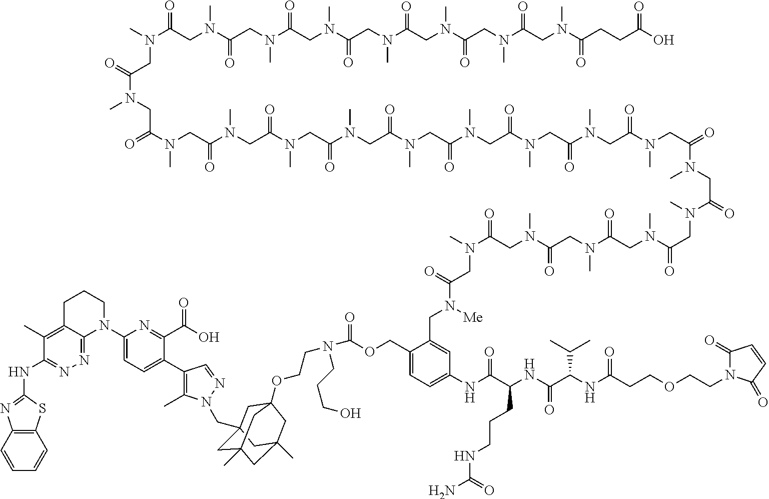 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L37A-P27 | 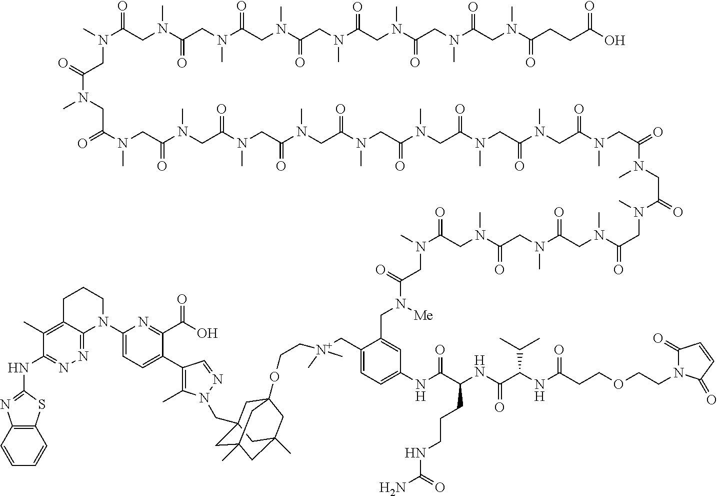 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L38A-P1 | 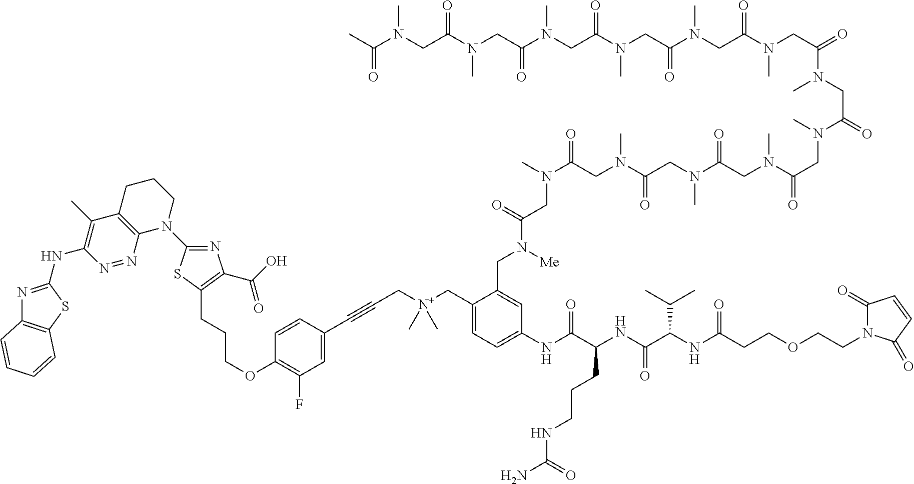 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1] |
| L38C-P19 | 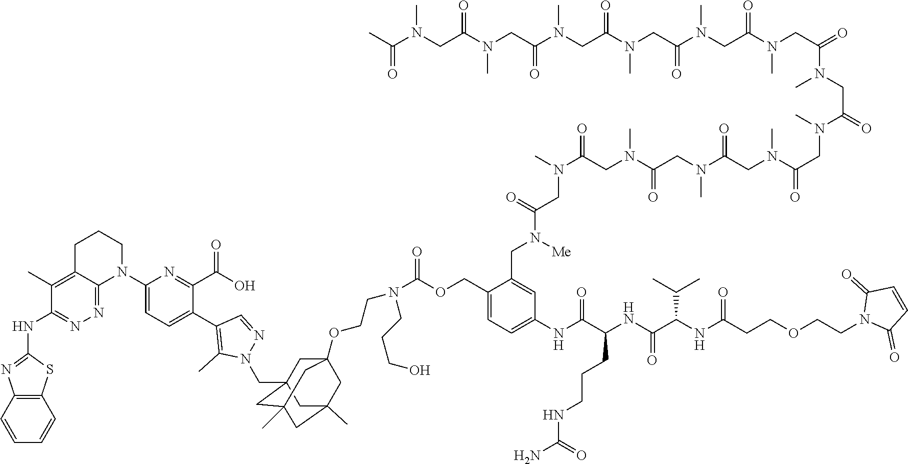 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCCO)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L38A-P21 | 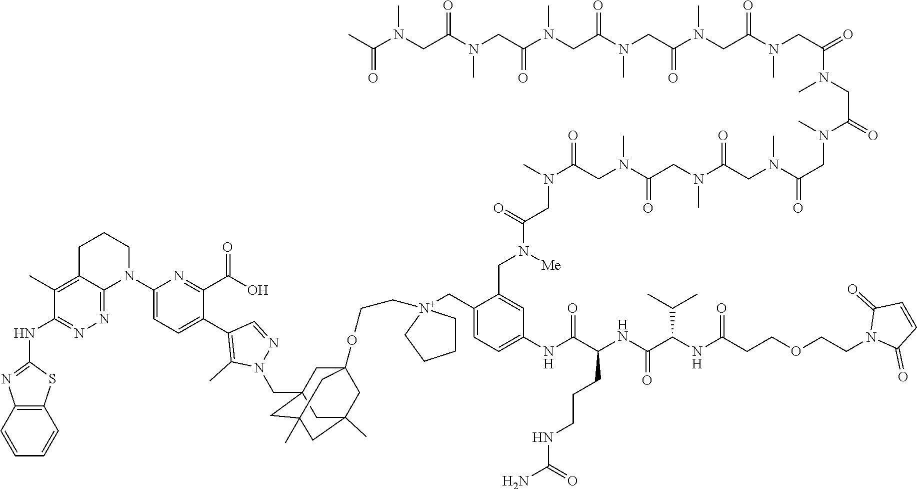 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1] |
| L38C-P25 | 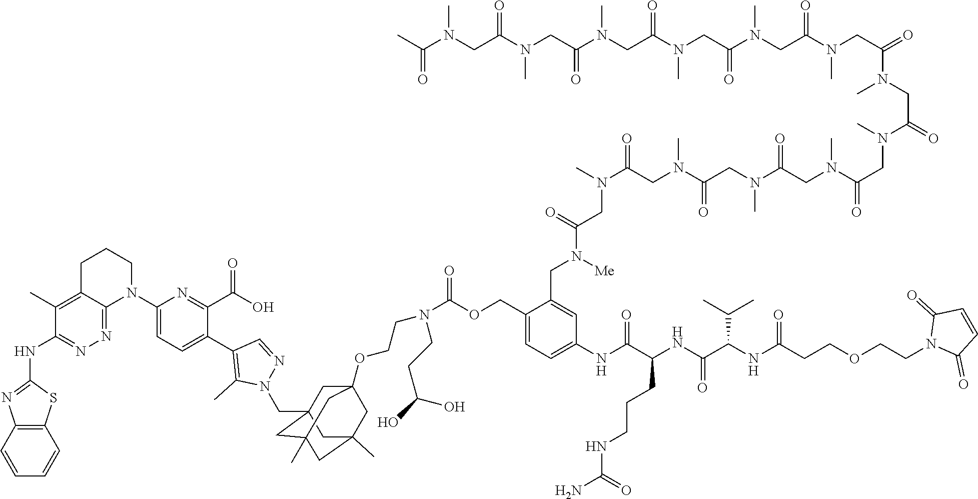 |
| L38A-P27 | 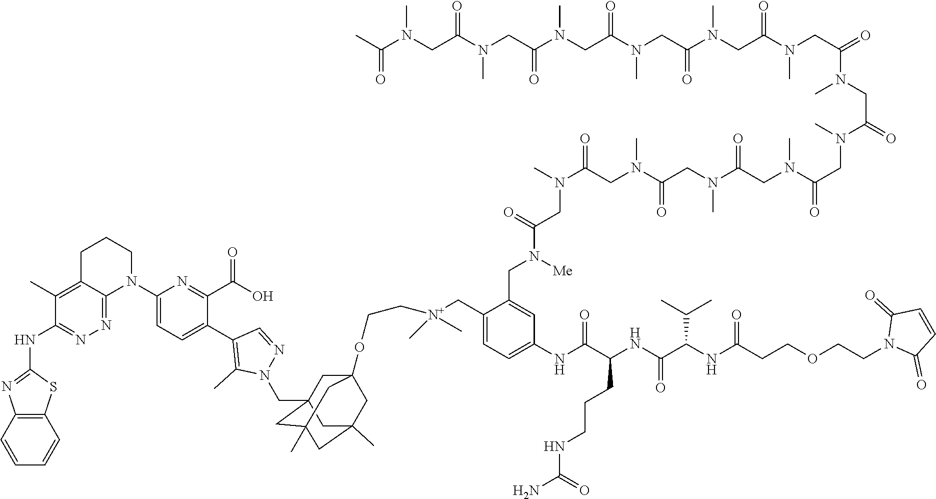 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L39A-P1 | 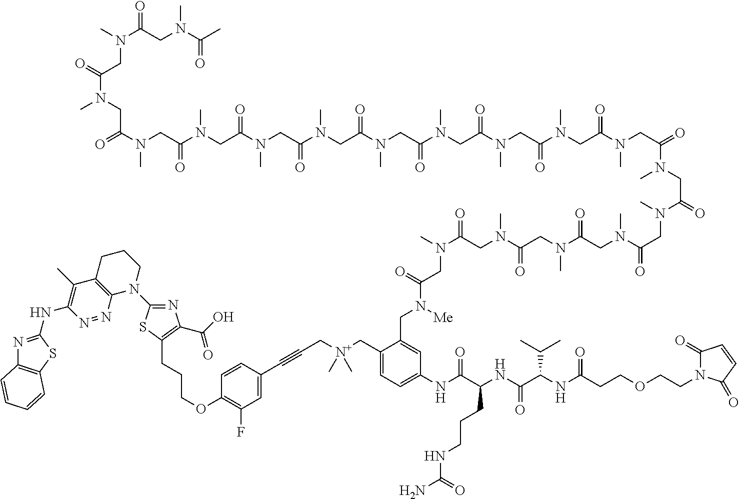 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1] |
| L39C-P19 | 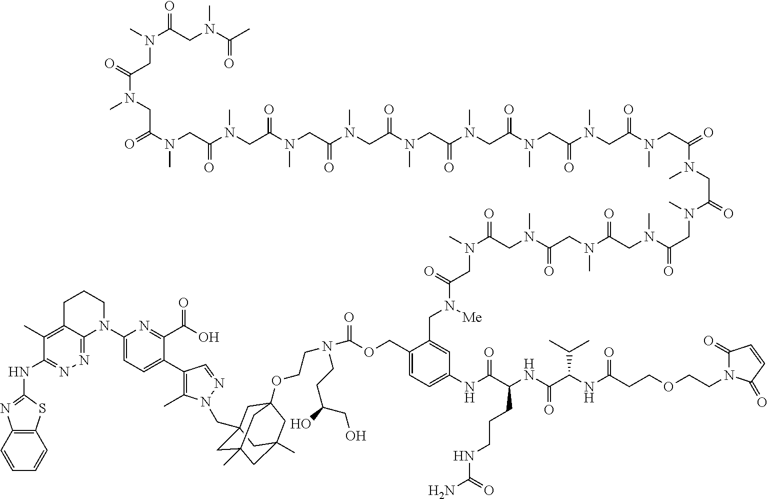 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO] |
| L39A-P21 | 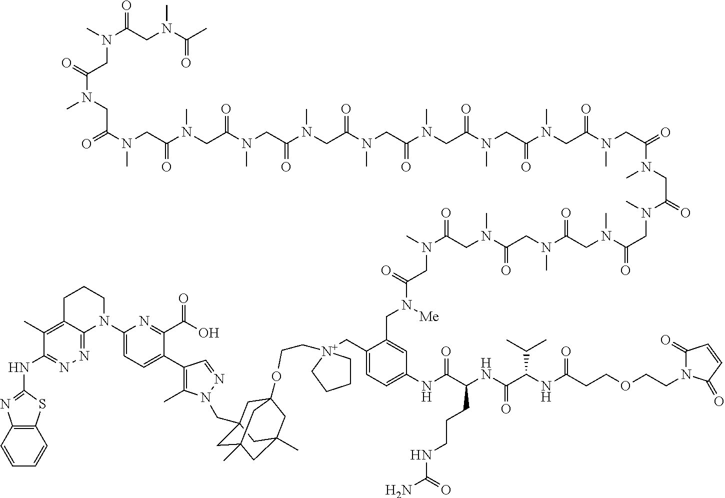 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1] |
| L39C-P25 | 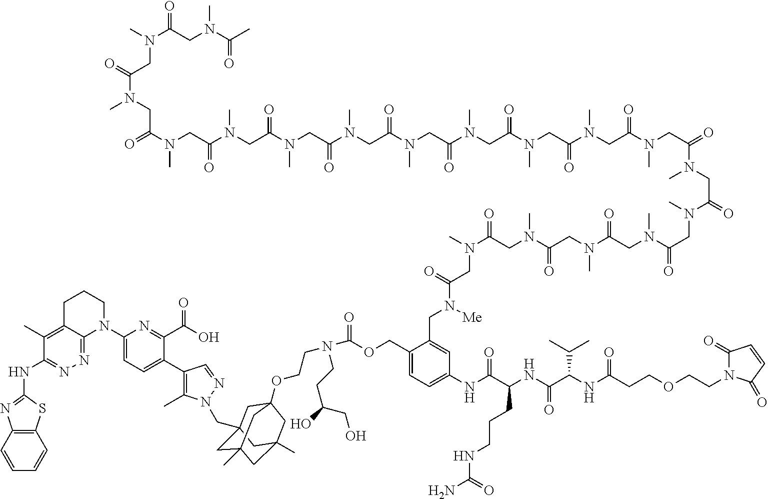 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO] |
| L39A-P27 | 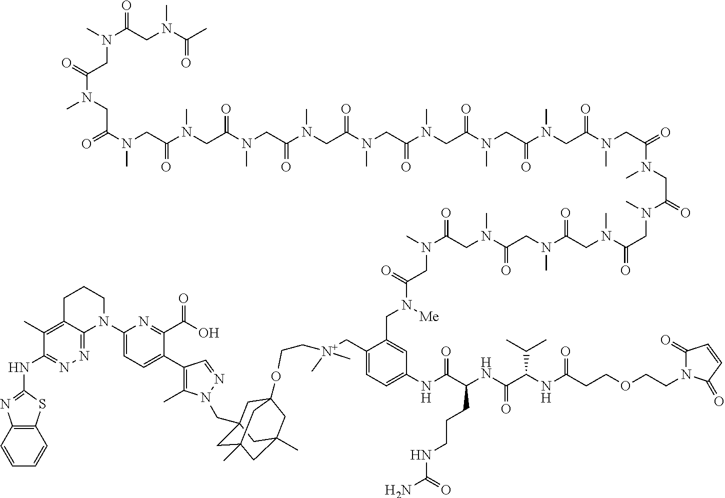 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L40A-P1 | 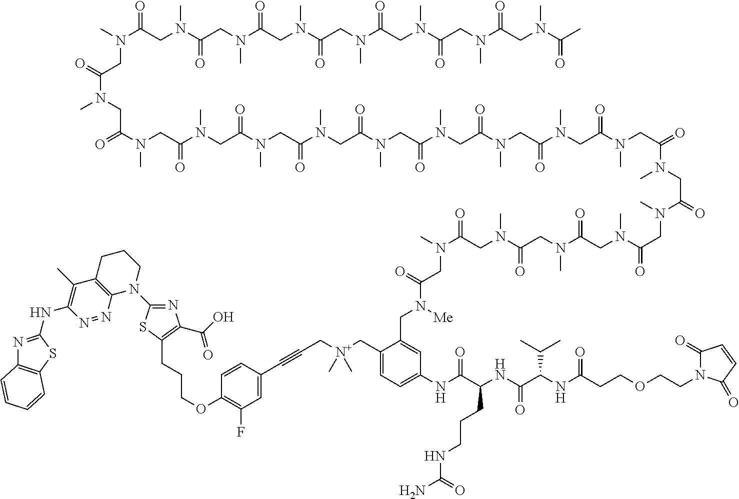 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1] |
| L40C-P19 | 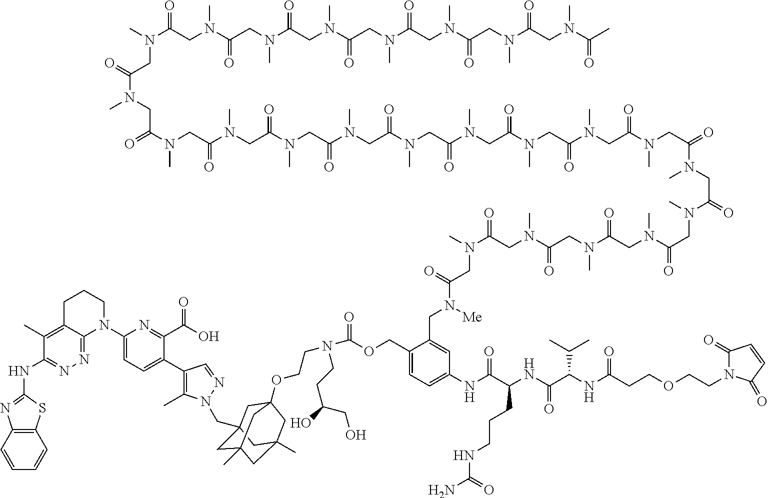 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO] |
| L40A-P21 | 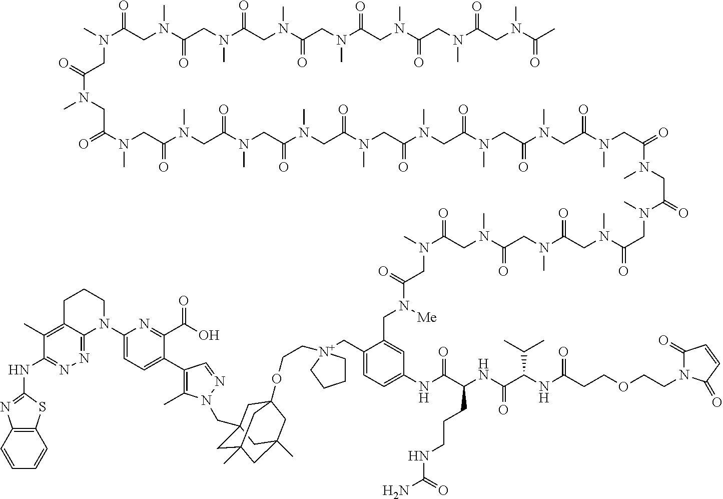 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1] |
| L40C-P25 | 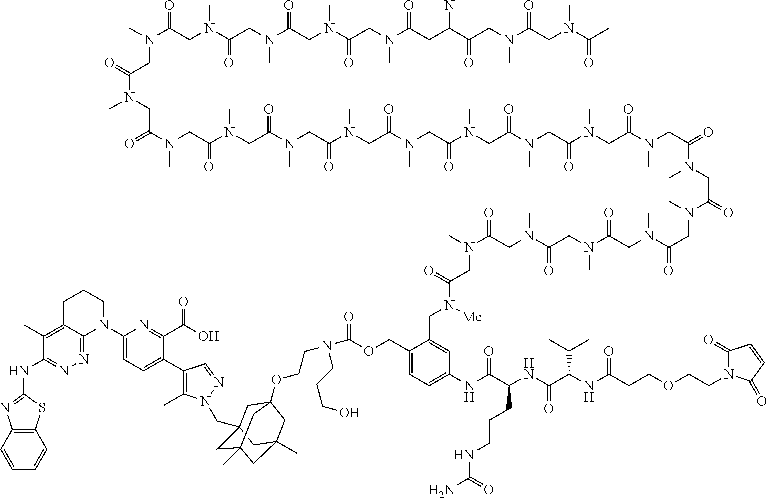 [SMILES: CC(=O)N(C)CC(=O)N(C)CC(=O)C(N)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCCO)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L40A-P27 | 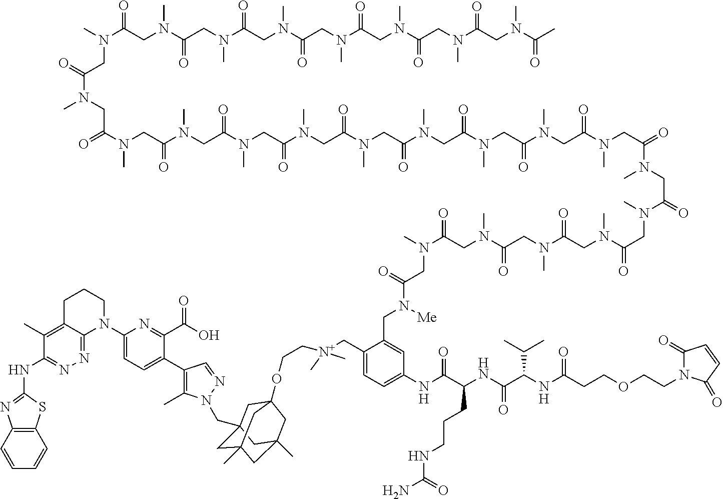 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L42A-P1 | 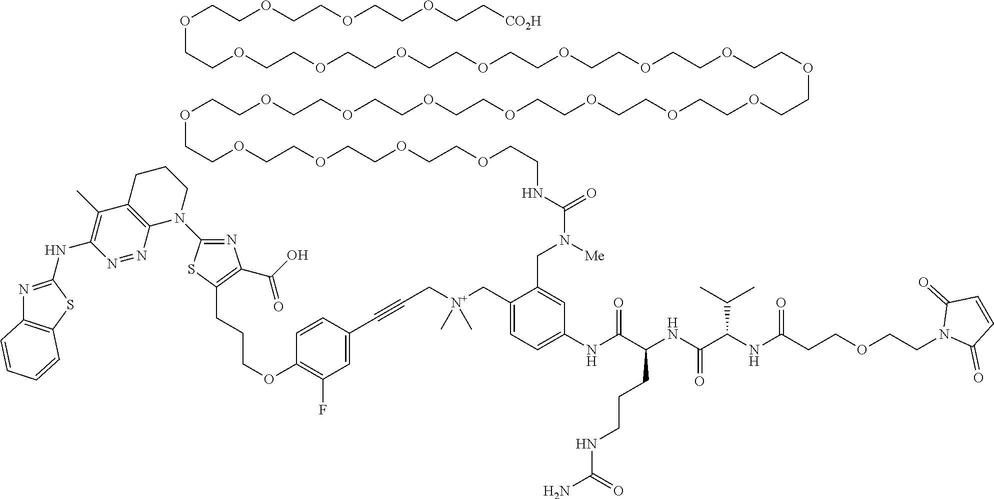 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)cc2F)s1] |
| L42C-P19 | 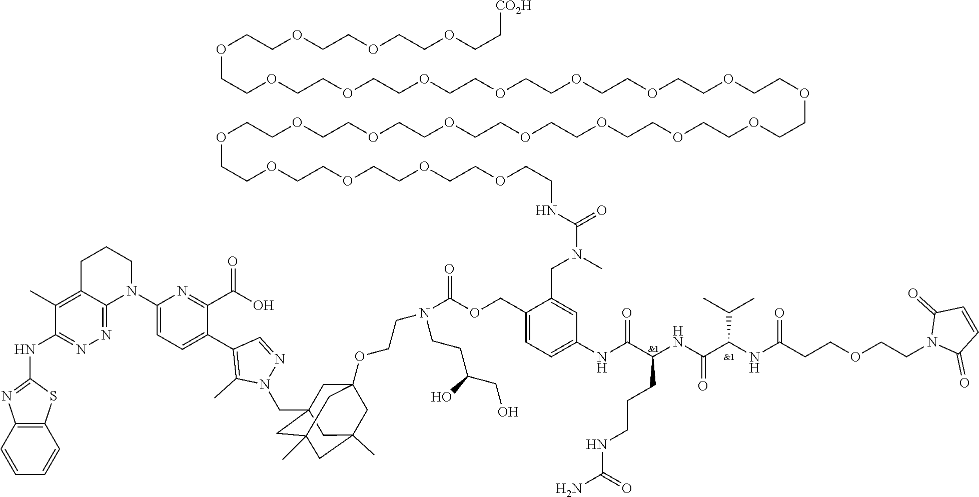 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1] |
| L42A-P21 | 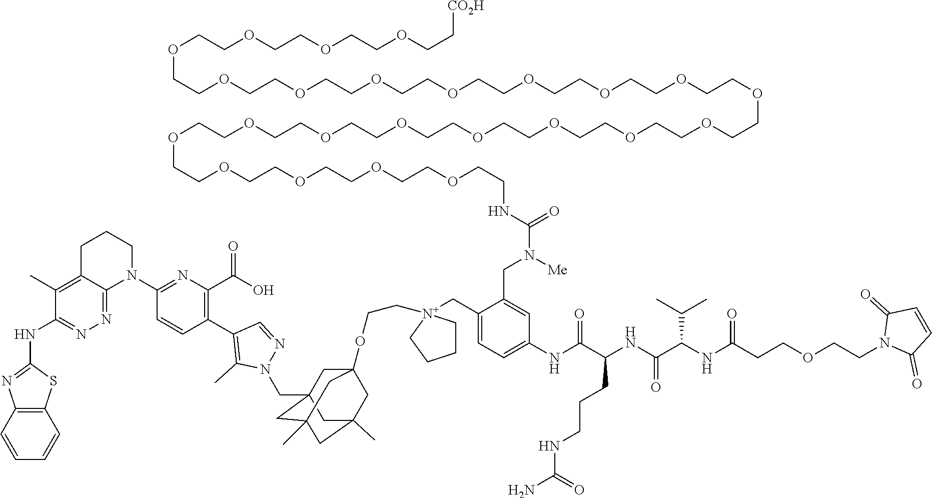 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CCCC3)(C5)C4)c2C)c(C(=O)O)n1] |
| L42C-P25 | 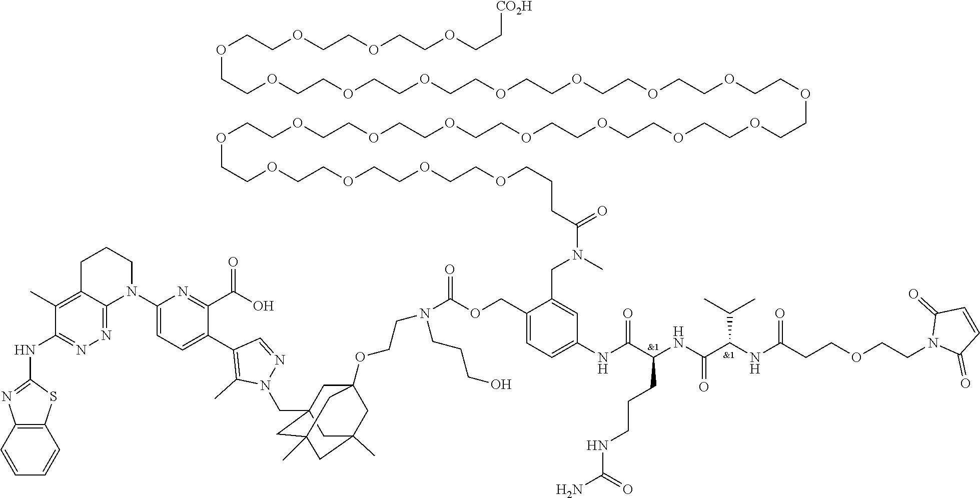 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1] |
| L42A-P27 | 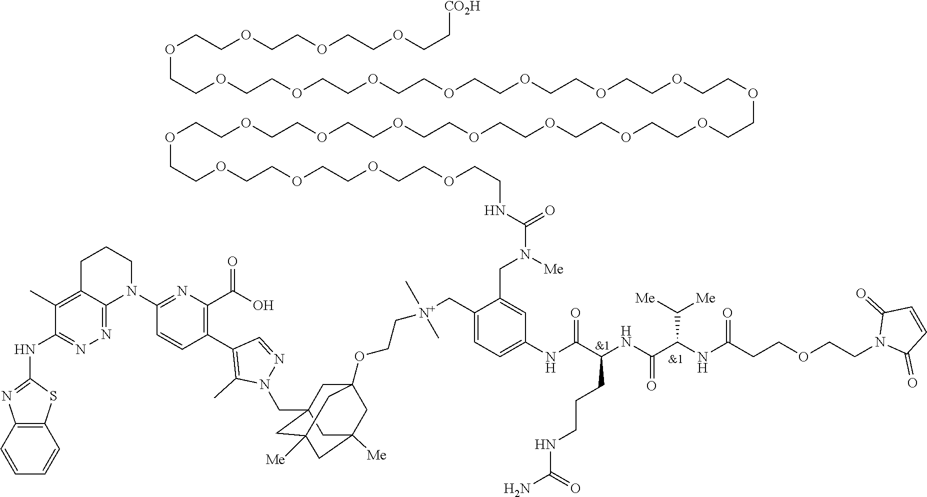 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1] |
| L67A-P1 | 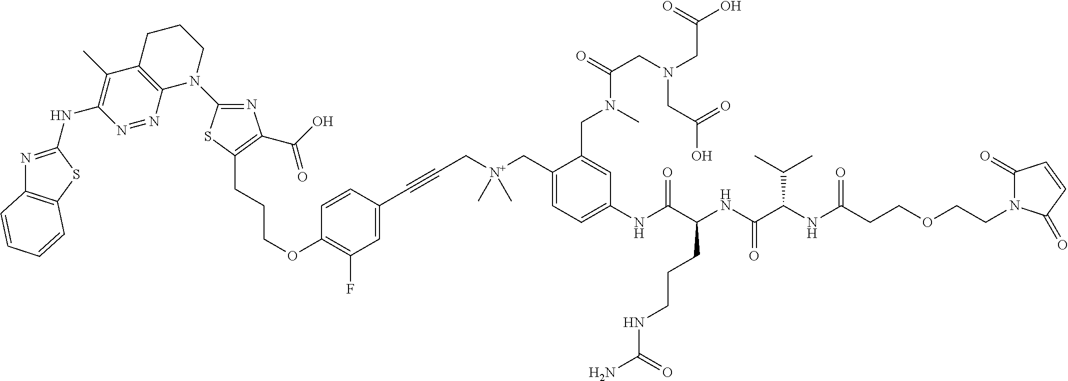 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)CN(CC(=O)O)CC(=O)O)cc2F)s1] |
| L67C-P19 | 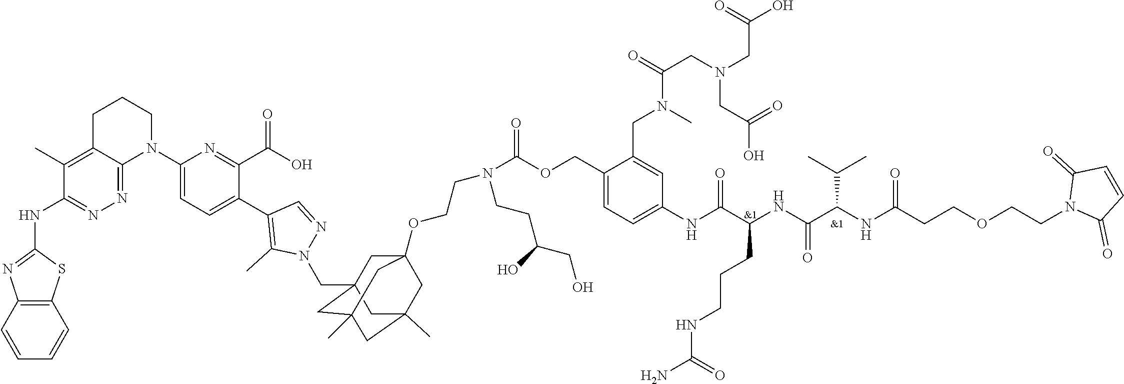 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CN(CC(=O)O)CC(=O)O)(C5)C4)c2C)c(C(=O)O)n1] |
| L67A-P21 | 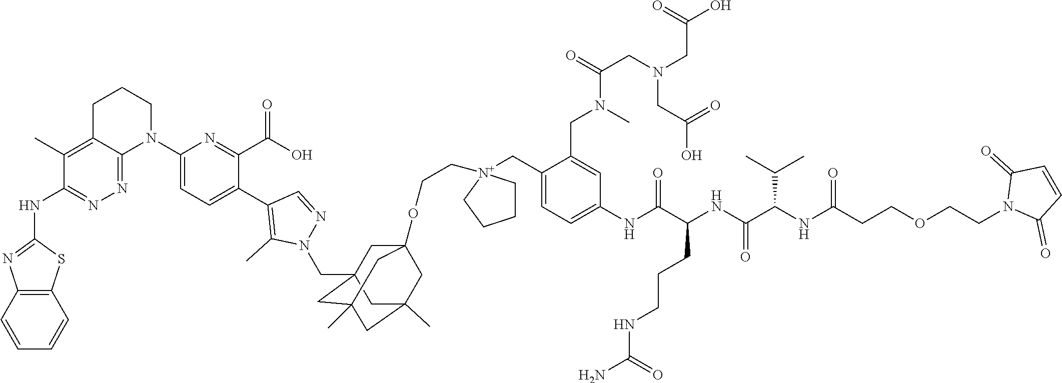 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)CN(CC(=O)O)CC(=O)O)CCCC3)(C5)C4)c2C)c(C(=O)O)n1] |
| L67C-P25 | 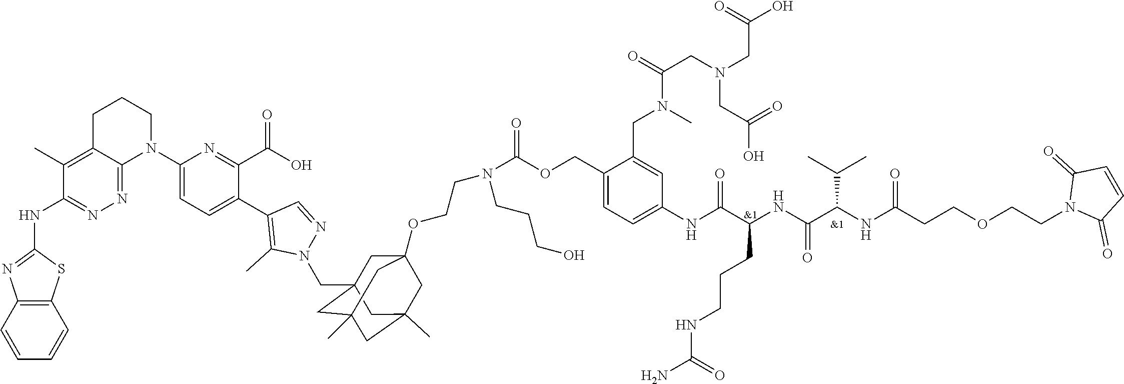 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CN(CC(=O)O)CC(=O)O)(C5)C4)c2C)c(C(=O)O)n1] |
| L67A-P27 | 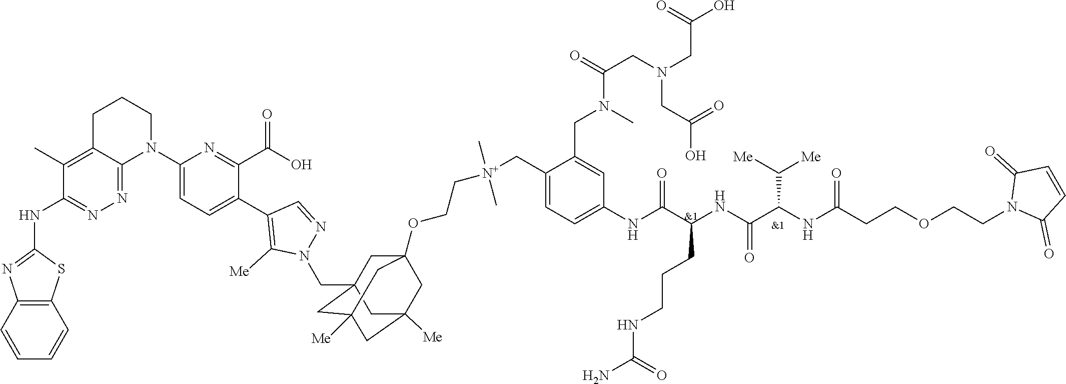 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CN(CC(=O)O)CC(=O)O)(C5)C4)c2C)c(C(=O)O)n1] |
| L100A-P1 | 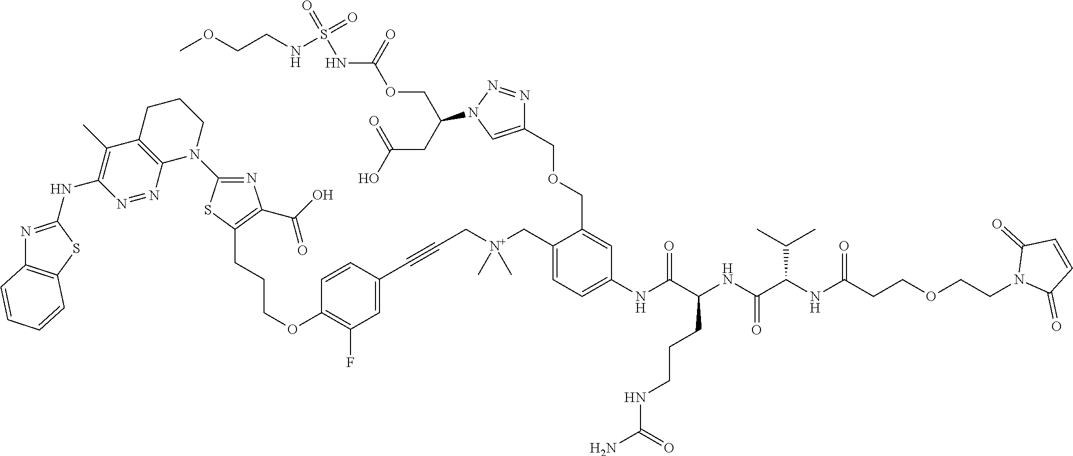 [SMILES: COCCNS(=O)(=O)NC(=O)OC[C@H](CC(=O)O)n1cc(COCc2cc(NC(=O)[C@H](CCCNC(N)=O)NC(=O)[C@@H](NC(=O)CCOCCN3C(=O)C=CC3=O)C(C)C)ccc2C[N+](C)(C)CC#Cc2ccc(OCCCc3sc(N4CCCc5c4nnc(Nc4nc6ccccc6s4)c5C)nc3C(=O)O)c(F)c2)nn1] |
| L100C-P19 | 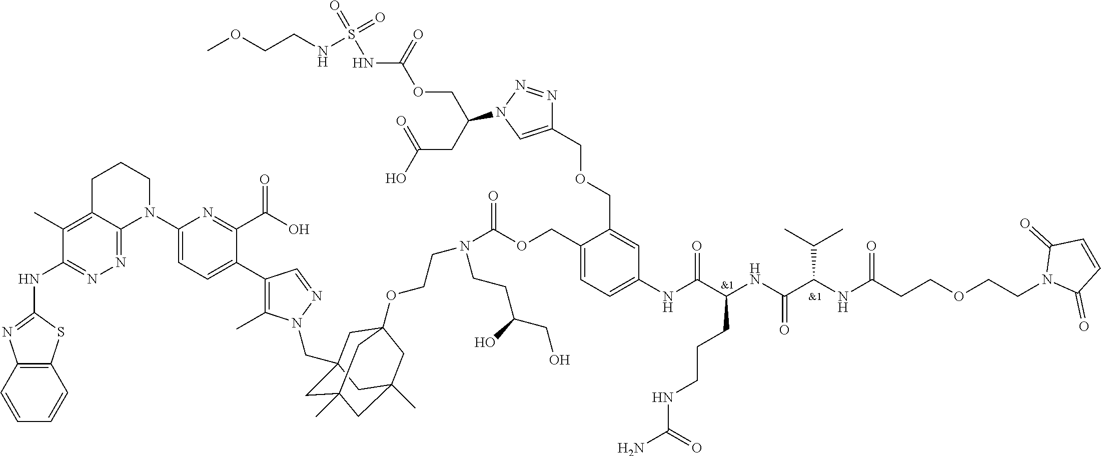 |
| L100A-P21 | 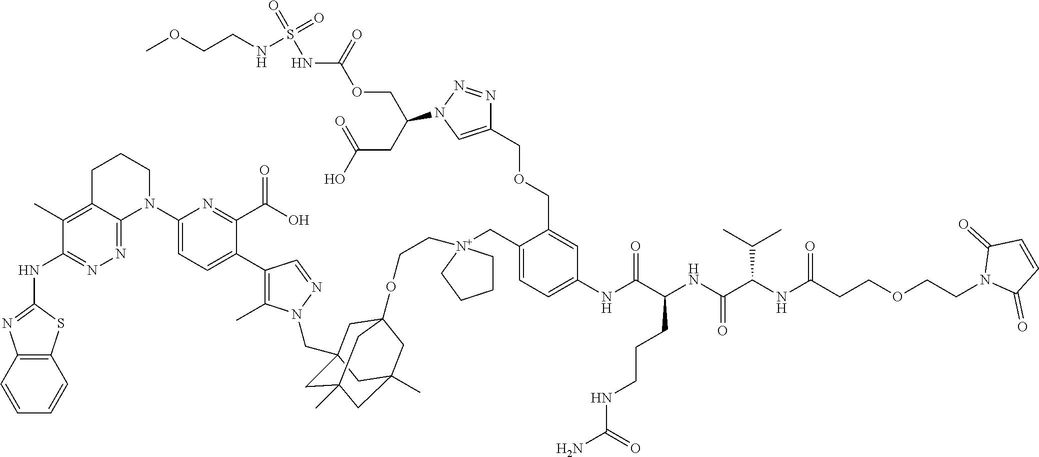 [SMILES: COCCNS(=O)(=O)NC(=O)OC[C@H](CC(=O)O)n1cc(COCc2cc(NC(=O)[C@H](CCCNC(N)=O)NC(=O)[C@@H](NC(=O)CCOCCN3C(=O)C=CC3=O)C(C)C)ccc2C[N+]2(CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CCCC2)nn1] |
| L100C-P25 | 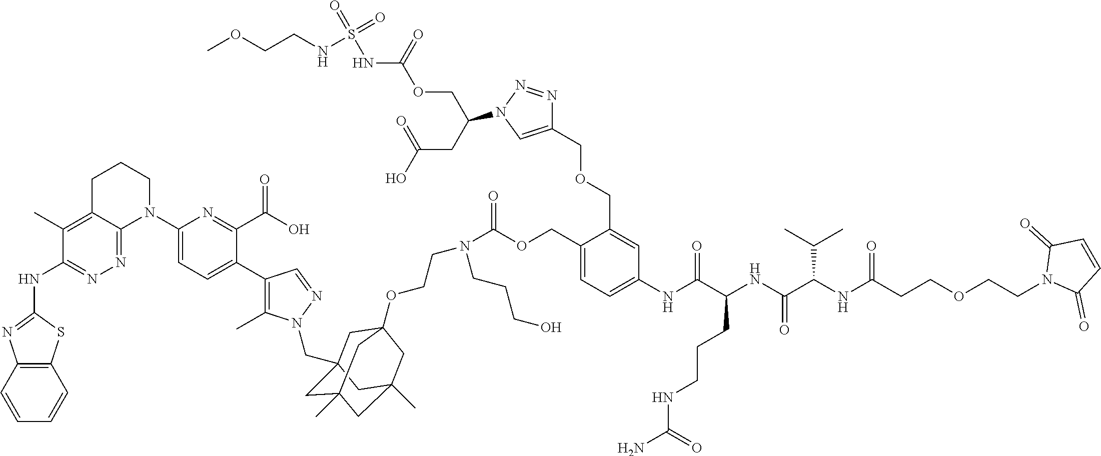 [SMILES: COCCNS(=O)(=O)NC(=O)OC[C@H](CC(=O)O)n1cc(COCc2cc(NC(=O)[C@H](CCCNC(N)=O)NC(=O)[C@@H](NC(=O)CCOCCN3C(=O)C=CC3=O)C(C)C)ccc2COC(=O)N(CCCO)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)nn1] |
| L100A-P27 | 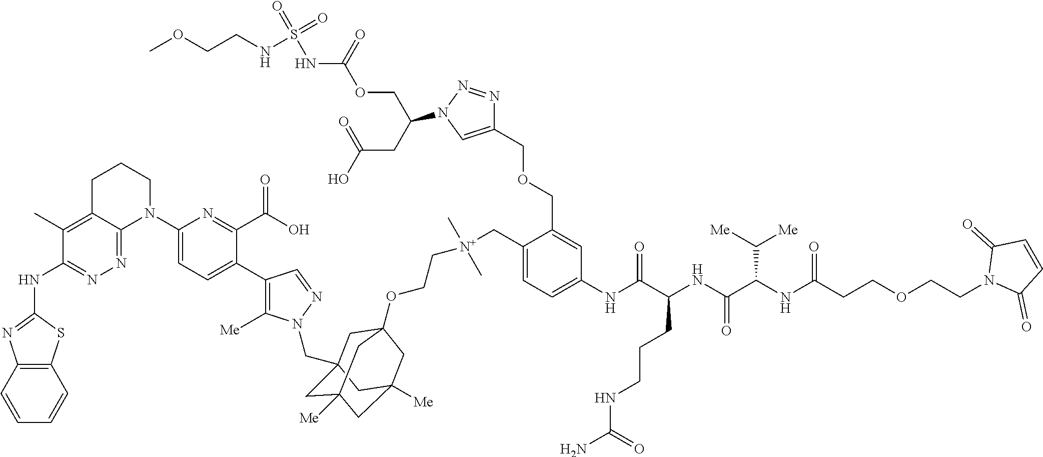 [SMILES: COCCNS(=O)(=O)NC(=O)OC[C@H](CC(=O)O)n1cc(COCc2cc(NC(=O)[C@H](CCCNC(N)=O)NC(=O)[C@@H](NC(=O)CCOCCN3C(=O)C=CC3=O)C(C)C)ccc2C[N+](C)(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)nn1] |
| L103A-P1 | 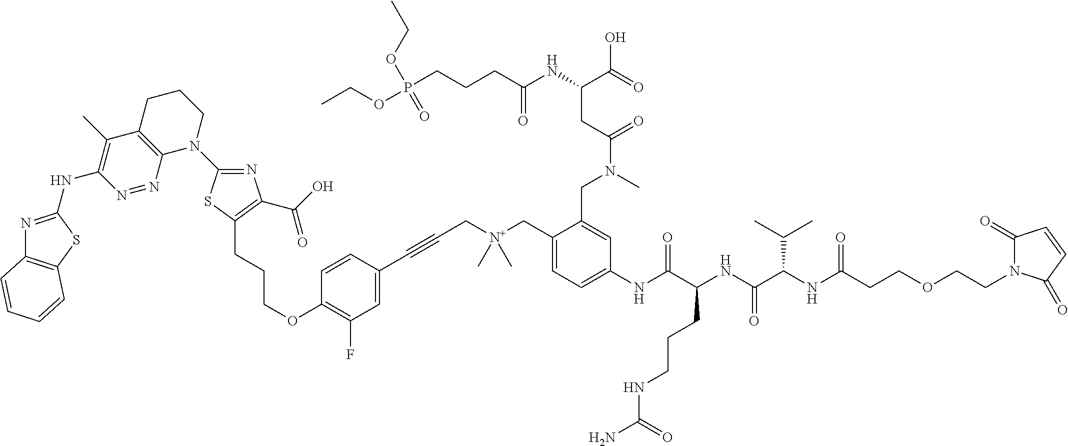 [SMILES: CCOP(=O)(CCCC(=O)N[C@@H](CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1)C(=O)O)OCC] |
| L103C-P19 | 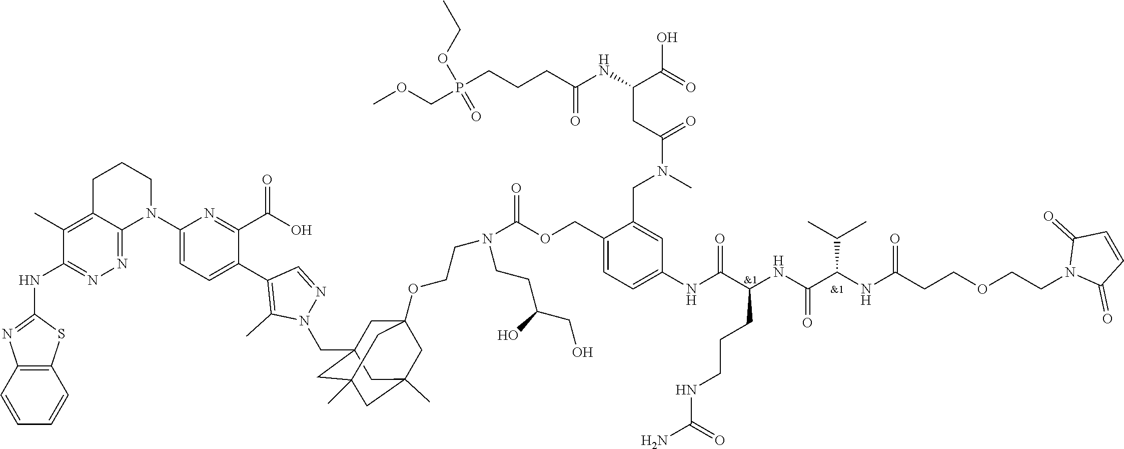 [SMILES: CCOP(=O)(CCCC(=O)N[C@@H](CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO)C(=O)O)COC] |
| L103A-P21 | 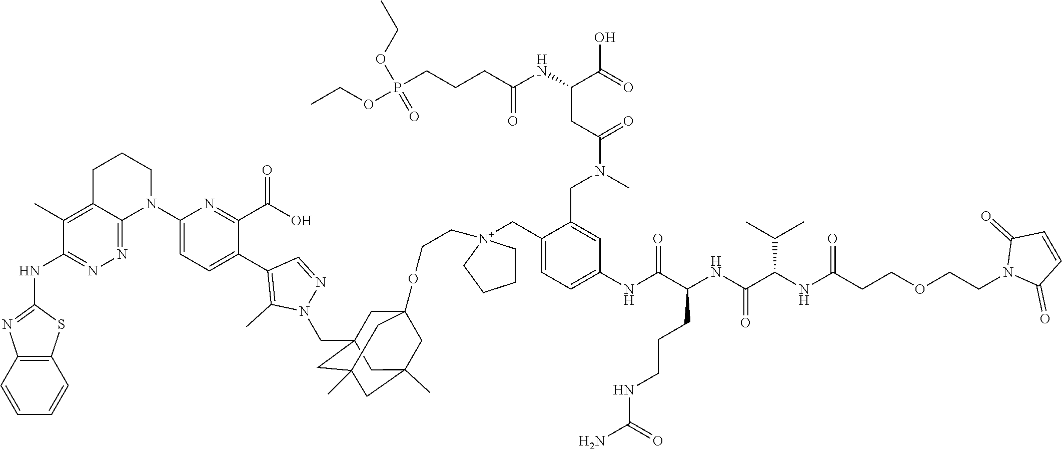 [SMILES: CCOP(=O)(CCCC(=O)N[C@@H](CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1)C(=O)O)OCC] |
| L103C-P25 | 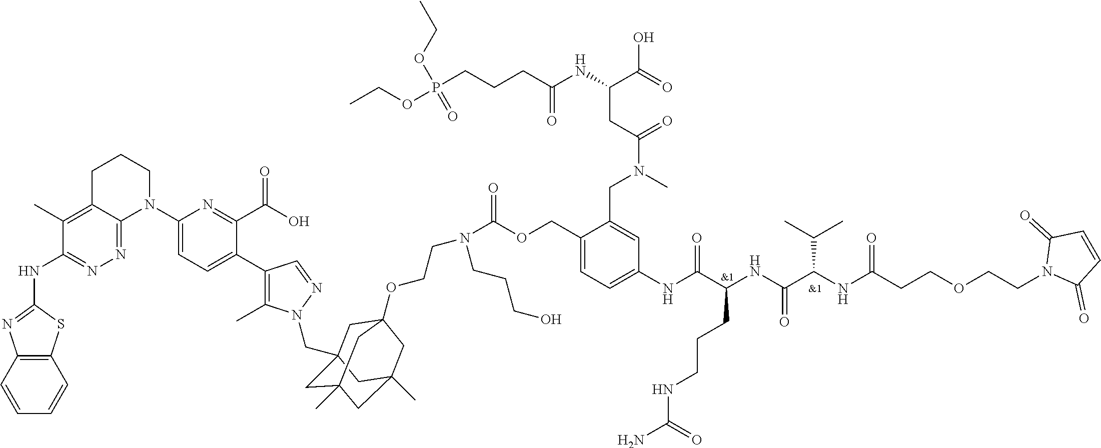 [SMILES: CCOP(=O)(CCCC(=O)N[C@@H](CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCCO)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)C(=O)O)OCC] |
| L103A-P27 | 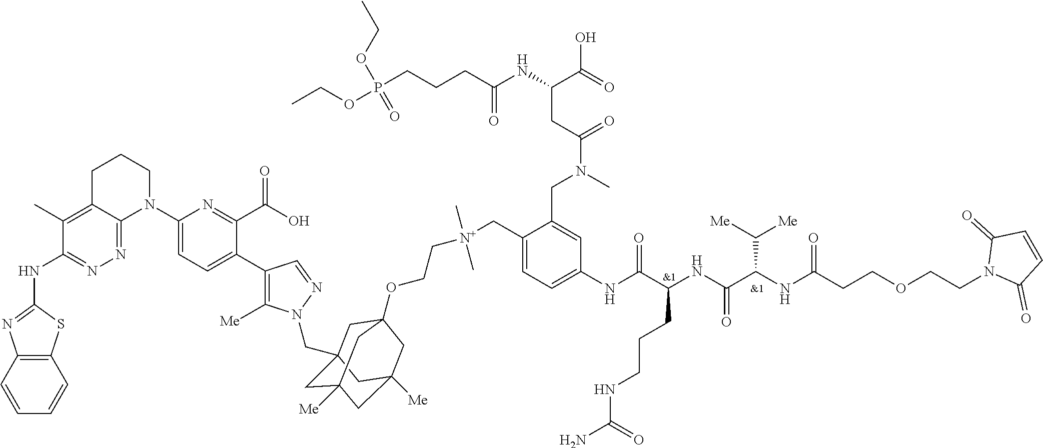 [SMILES: CCOP(=O)(CCCC(=O)N[C@@H](CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)C(=O)O)OCC] |
| L106A-P2 | 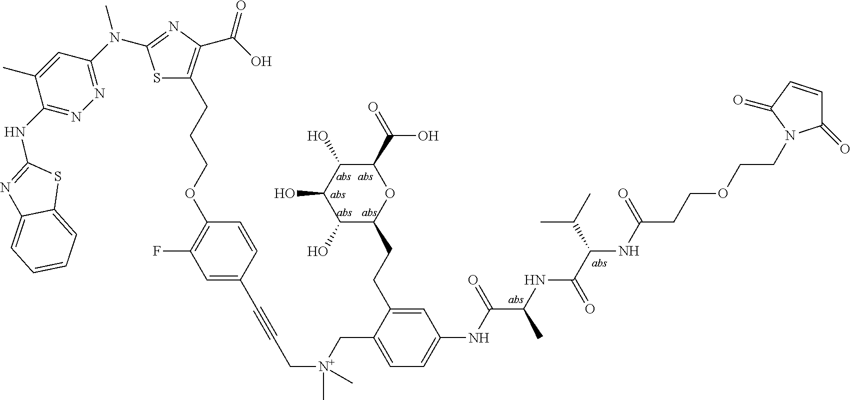 [SMILES: Cc1cc(N(C)c2nc(C(=O)O)c(CCCOc3ccc(C#CC[N+](C)(C)Cc4ccc(NC(=O)[C@H](C)NC(=O)[C@@H](NC(=O)CCOCCN5C(=O)C=CC5=O)C(C)C)cc4CC[C@@H]4O[C@H](C(=O)O)[C@@H](O)[C@H](O)[C@H]4O)cc3F)s2)nnc1Nc1nc2ccccc2s1] |
| L106C-P7 | 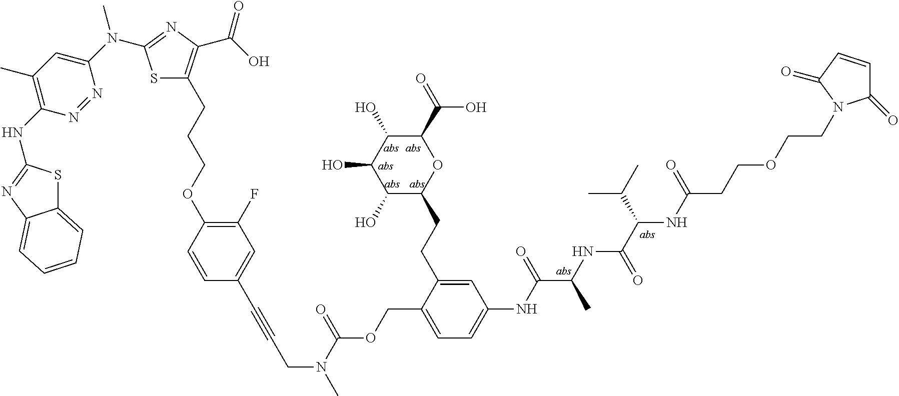 [SMILES: Cc1cc(N(C)c2nc(C(=O)O)c(CCCOc3ccc(C#CCN(C)C(=O)OCc4ccc(NC(=O)[C@H](C)NC(=O)[C@@H](NC(=O)CCOCCN5C(=O)C=CC5=O)C(C)C)cc4CC[C@@H]4O[C@H](C(=O)O)[C@@H](O)[C@H](O)[C@H]4O)cc3F)s2)nnc1Nc1nc2ccccc2s1] |
| L107C-P7 | 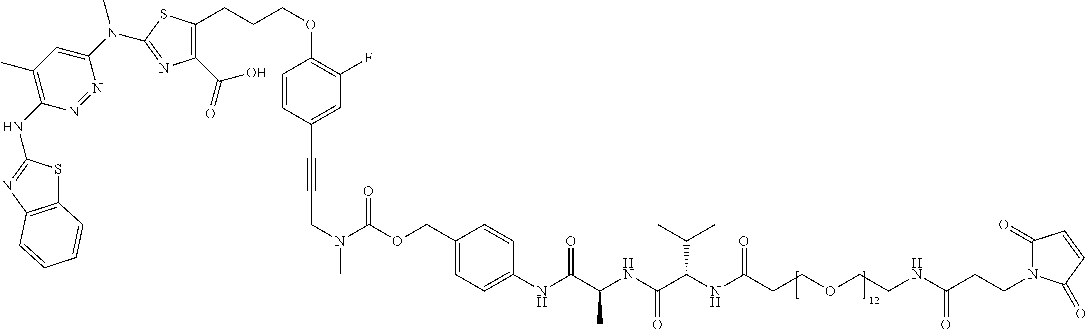 |
| L107A-P2 | 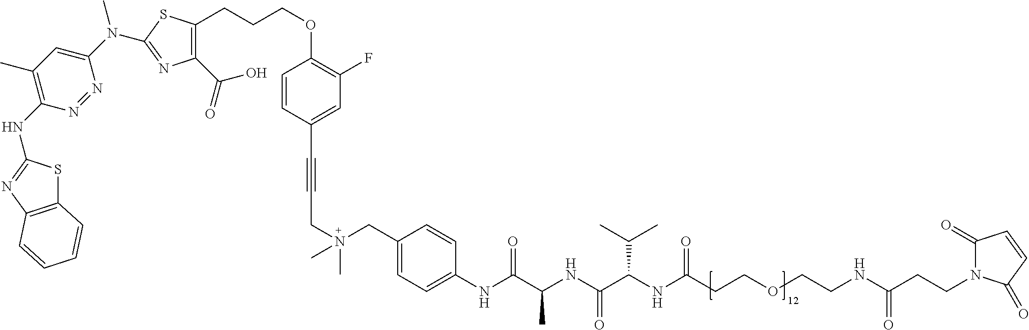 |
| L108A-P2 | 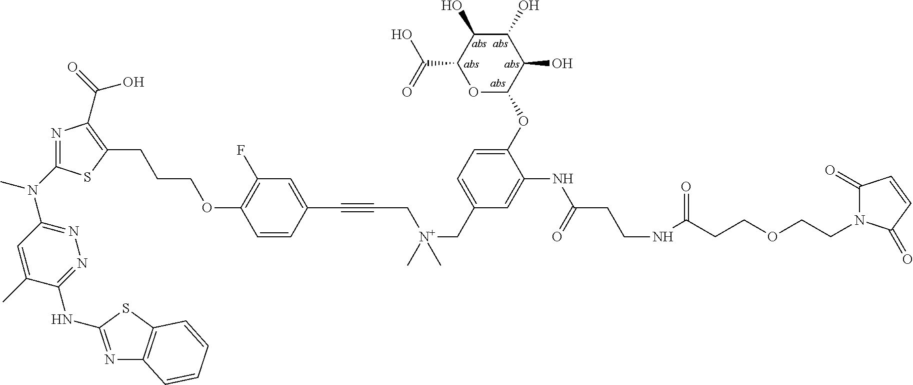 [SMILES: Cc1cc(N(C)c2nc(C(=O)O)c(CCCOc3ccc(C#CC[N+](C)(C)Cc4ccc(O[C@@H]5O[C@H](C(=O)O)[C@@H](O)[C@H](O)[C@H]5O)c(NC(=O)CCNC(=O)CCOCCN5C(=O)C=CC5=O)c4)cc3F)s2)nnc1Nc1nc2ccccc2s1] |
| L109A-P1 | 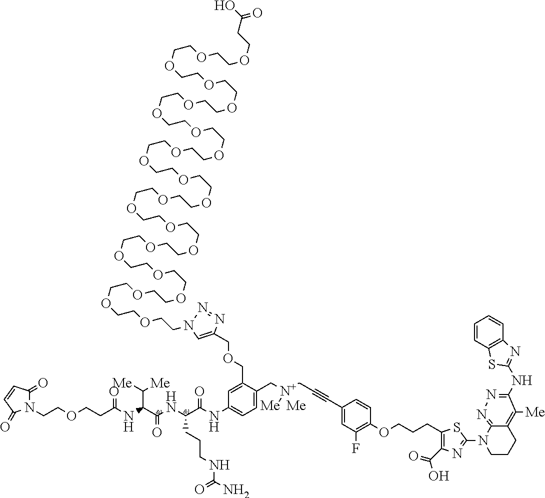 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3COCc3cn(CCOCCOCCOCCOCCOCCOCCOCCOCCOCCOCCOCCOCCOCCOCCOCCOCCOCCOCCOCCOCCOCCOCCOCCOCCC(=O)O)nn3)cc2F)s1] |
| L110C-P7 | 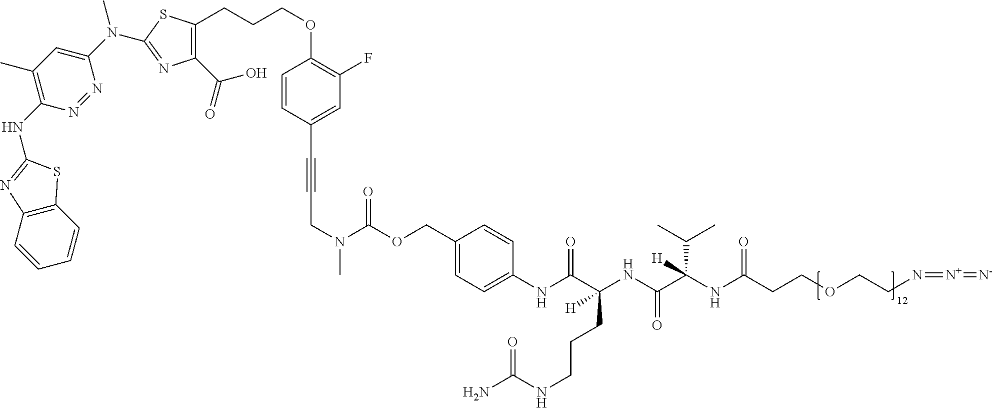 |
| L111A-P1 | 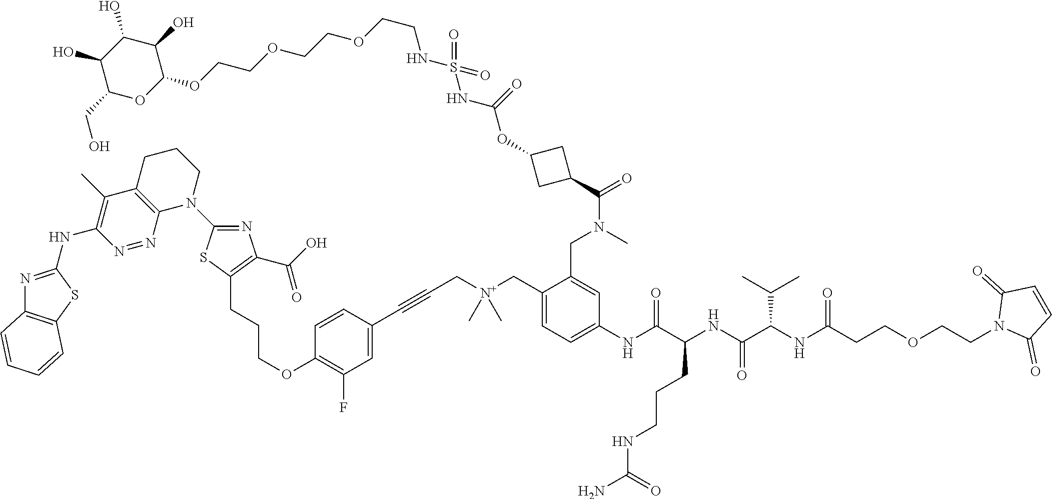 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)[C@H]3C[C@H](OC(=O)NS(=O)(=O)NCCOCCOCCO[C@@H]4O[C@H](CO)[C@@H](O)[C@H](O)[C@H]4O)C3)cc2F)s1] |
| L111C-P19 | 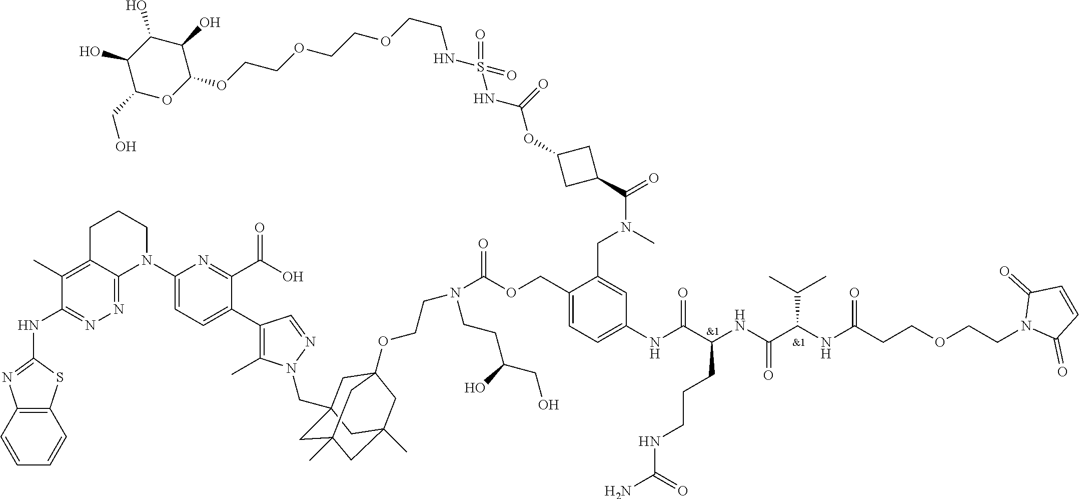 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)[C@H]3C[C@H](OC(=O)NS(=O)(=O)NCCOCCOCCO[C@@H]6O[C@H](CO)[C@@H](O)[C@H](O)[C@H]6O)C3)(C5)C4)c2C)c(C(=O)O)n1] |
| L111A-P21 | 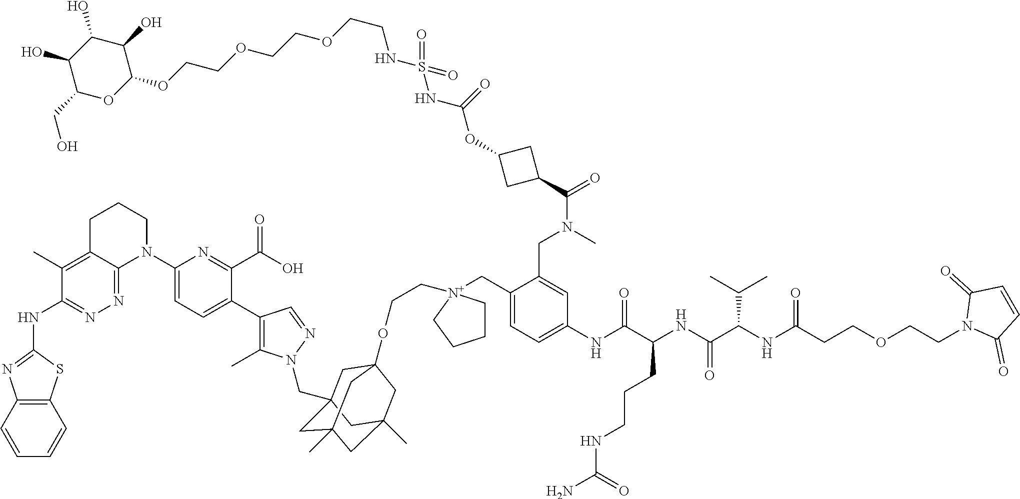 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)[C@H]6C[C@H](OC(=O)NS(=O)(=O)NCCOCCOCCO[C@@H]7O[C@H](CO)[C@@H](O)[C@H](O)[C@H]7O)C6)CCCC3)(C5)C4)c2C)c(C(=O)O)n1] |
| L111C-P25 | 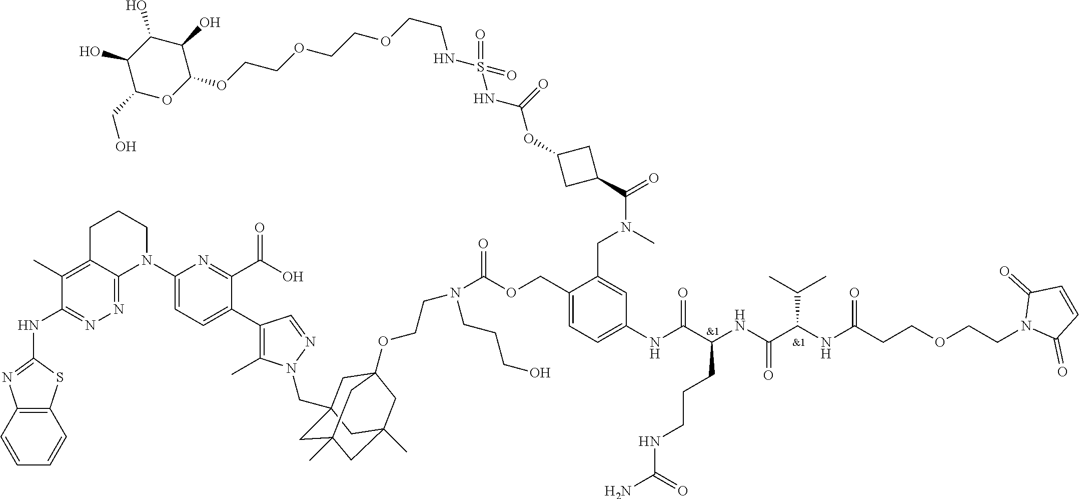 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)[C@H]3C[C@H](OC(=O)NS(=O)(=O)NCCOCCOCCO[C@@H]6O[C@H](CO)[C@@H](O)[C@H](O)[C@H]6O)C3)(C5)C4)c2C)c(C(=O)O)n1] |
| L111A-P27 | 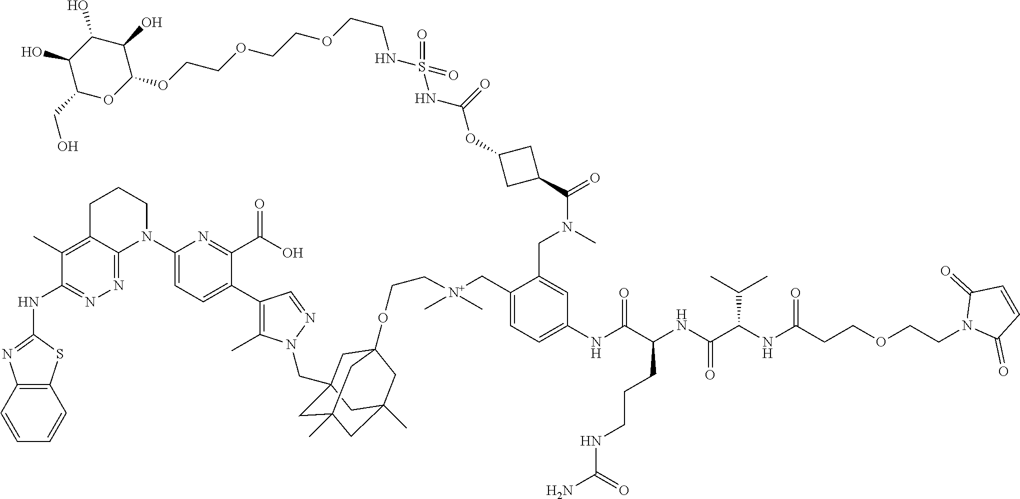 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)[C@H]3C[C@H](OC(=O)NS(=O)(=O)NCCOCCOCCO[C@@H]6O[C@H](CO)[C@@H](O)[C@H](O)[C@H]6O)C3)(C5)C4)c2C)c(C(=O)O)n1] |
| L112A-P1 | 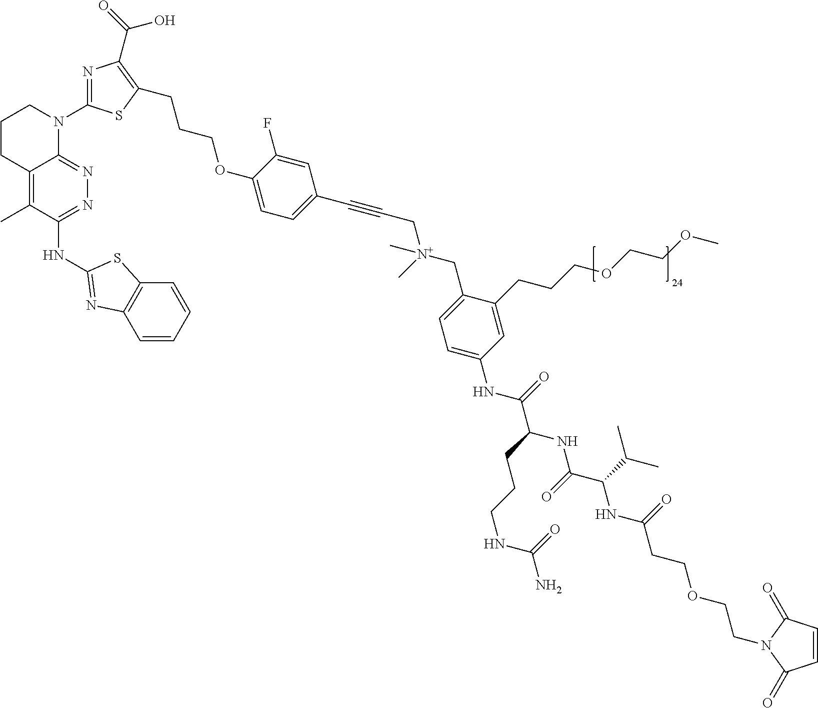 |

In some embodiments, the antibody-drug conjugate has a formula according to any one of the structures shown in Table 1.

TABLE 1
Exemplary ADC Structures

| ADC | |
|---|---|
| Name | ADC Structure |
| Ab- L11C- P25 | 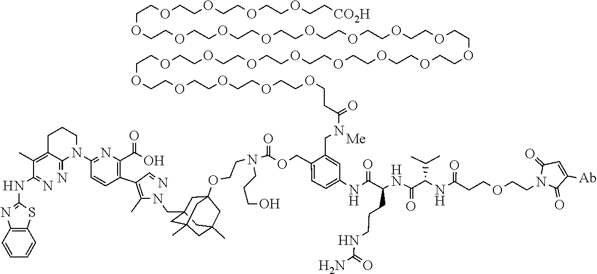 |
| Ab- L11A- P21 | 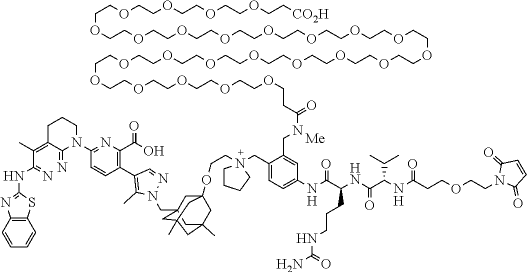 |
| Ab- L42C- P25 | 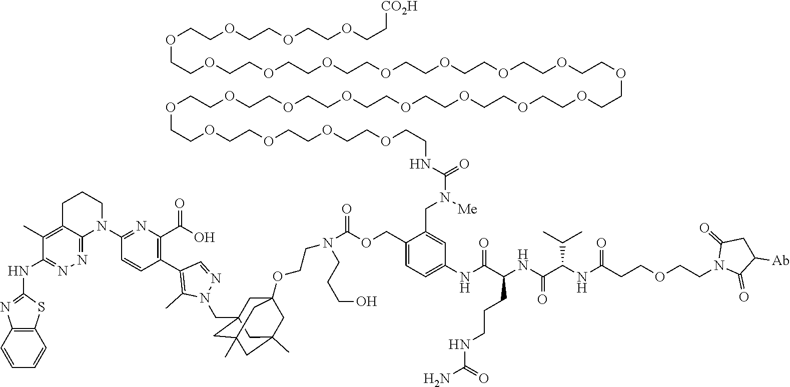 |

*Ab: Any antibody described herein (e.g., EphA2-DANAPA antibody)

The ADCs depicted above can also be represented by the following formula:

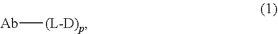

wherein Ab represents an anti-EphA2 antibody or an antigen fragment thereof covalently linked to the linker-payload (L/P) depicted above; p is an integer from 1 to 16. In some embodiments, p is an integer from 1 to 8. In some embodiments, p is an integer from 1 to 5. In some embodiments, p is an integer from 2 to 4. In some embodiments, p is 2. In some embodiments, p is 4. In some embodiments, p is determined by liquid chromatography-mass spectrometry (LC-MS).

As used herein, “L/P” refers to the linker-payloads, linker-drugs, or linker-compounds disclosed herein and the terms “L #-P #” and “L #-C #” are used interchangeably to refer to a specific linker-drug disclosed herein, while the codes “P #” and “C #” are used interchangeably to refer to a specific compound unless otherwise specified. For example, both “L1-C1” and “L1-P1” refer to the same linker-payload structure disclosed herein, while both “C1” and “P1” indicate the same compound disclosed herein, including an enantiomer, diastereoisomer, atropisomer, deuterated derivative, and/or pharmaceutically acceptable salt of any of the foregoing.

Also provided herein, in some embodiments, are compositions comprising multiple copies of an antibody-drug conjugate (e.g., any of the exemplary antibody-drug conjugates described herein). In some embodiments, the average p of the antibody-drug conjugates in the composition is from about 2 to about 4.

Also provided herein, in some embodiments, are pharmaceutical compositions comprising an antibody-drug conjugate (e.g., any of the exemplary antibody-drug conjugates described herein) or a composition (e.g., any of the exemplary compositions described herein), and a pharmaceutically acceptable carrier.

Further provided herein, in some embodiments, are therapeutic uses for the described ADC compounds and compositions, e.g., in treating a cancer. In some embodiments, the present disclosure provides methods of treating a cancer (e.g., a cancer that expresses the EphA2 antigen targeted by the antibody or antigen-binding fragment of the ADC). In some embodiments, the present disclosure provides methods of reducing or slowing the expansion of a cancer cell population in a subject. In some embodiments, the present disclosure provides methods of determining whether a subject having or suspected of having a cancer will be responsive to treatment with an ADC compound or composition disclosed herein.

An exemplary embodiment is a method of treating a subject having or suspected of having a cancer, comprising administering to the subject a therapeutically effective amount of an antibody-drug conjugate, composition, or pharmaceutical composition (e.g., any of the exemplary antibody-drug conjugates, compositions, or pharmaceutical compositions disclosed herein). In some embodiments, the cancer expresses the target antigen EphA2. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer. In some embodiments, the cancer is breast cancer, non-small cell lung cancer, pancreatic cancer, esophageal cancer, head and neck cancer, stomach cancer, bladder cancer, or colon cancer. In some embodiments, the cancer is breast cancer or non-small cell lung cancer.

Another exemplary embodiment is a method of reducing or inhibiting the growth of a tumor in a subject, comprising administering to the subject a therapeutically effective amount of an antibody-drug conjugate, composition, or pharmaceutical composition (e.g., any of the exemplary antibody-drug conjugates, compositions, or pharmaceutical compositions disclosed herein). In some embodiments, the tumor expresses the target antigen EphA2. In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, pancreatic cancer, stomach cancer, colon cancer, or spleen cancer. In some embodiments, the tumor is a gastric cancer. In some embodiments, the tumor is breast cancer, non-small cell lung cancer, pancreatic cancer, esophageal cancer, head and neck cancer, stomach cancer, bladder cancer, or colon cancer. In some embodiments, the tumor is breast cancer or non-small cell lung cancer. In some embodiments, administration of the antibody-drug conjugate, composition, or pharmaceutical composition reduces or inhibits the growth of the tumor by at least about 10%, at least about 20%, at least about 30%, at least about 40%, at least about 50%, at least about 60%, at least about 70%, at least about 80%, at least about 90%, at least about 95%, or at least about 99%.

Another exemplary embodiment is a method of reducing or slowing the expansion of a cancer cell population in a subject, comprising administering to the subject a therapeutically effective amount of an antibody-drug conjugate, composition, or pharmaceutical composition (e.g., any of the exemplary antibody-drug conjugates, compositions, or pharmaceutical compositions disclosed herein). In some embodiments, the cancer cell population expresses the target antigen EphA2.

In some embodiments, the cancer cell population is from a tumor or a hematological cancer. In some embodiments, the cancer cell population is from a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer cell population is from a lymphoma or gastric cancer. In some embodiments, the cancer cell population is from breast cancer, non-small cell lung cancer, pancreatic cancer, esophageal cancer, head and neck cancer, stomach cancer, bladder cancer, or colon cancer. In some embodiments, the cancer cell population is from breast cancer or non-small cell lung cancer. In some embodiments, administration of the antibody-drug conjugate, composition, or pharmaceutical composition reduces the cancer cell population by at least about 10%, at least about 20%, at least about 30%, at least about 40%, at least about 50%, at least about 60%, at least about 70%, at least about 80%, at least about 90%, at least about 95%, or at least about 99%. In some embodiments, administration of the antibody-drug conjugate, composition, or pharmaceutical composition slows the expansion of the cancer cell population by at least about 10%, at least about 20%, at least about 30%, at least about 40%, at least about 50%, at least about 60%, at least about 70%, at least about 80%, at least about 90%, at least about 95%, or at least about 99%.

Another exemplary embodiment is an antibody-drug conjugate, composition, or pharmaceutical composition (e.g., any of the exemplary antibody-drug conjugates, compositions, or pharmaceutical compositions disclosed herein) for use in treating a subject having or suspected of having a cancer. In some embodiments, the cancer expresses the target antigen EphA2. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer. In some embodiments, the cancer cell population is from breast cancer, non-small cell lung cancer, pancreatic cancer, esophageal cancer, head and neck cancer, stomach cancer, bladder cancer, or colon cancer. In some embodiments, the cancer cell population is from breast cancer or non-small cell lung cancer.

Another exemplary embodiment is a use of an antibody-drug conjugate, composition, or pharmaceutical composition (e.g., any of the exemplary antibody-drug conjugates, compositions, or pharmaceutical compositions disclosed herein) in treating a subject having or suspected of having a cancer. In some embodiments, the cancer expresses the target antigen EphA2. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer. In some embodiments, the cancer is breast cancer, non-small cell lung cancer, pancreatic cancer, esophageal cancer, head and neck cancer, stomach cancer, bladder cancer, or colon cancer. In some embodiments, the cancer is breast cancer or non-small cell lung cancer.

Another exemplary embodiment is a use of an antibody-drug conjugate, composition, or pharmaceutical composition (e.g., any of the exemplary antibody-drug conjugates, compositions, or pharmaceutical compositions disclosed herein) in a method of manufacturing a medicament for treating a subject having or suspected of having a cancer. In some embodiments, the cancer expresses the target antigen EphA2. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer. In some embodiments, the cancer is breast cancer, non-small cell lung cancer, pancreatic cancer, esophageal cancer, head and neck cancer, stomach cancer, bladder cancer, or colon cancer. In some embodiments, the cancer is breast cancer or non-small cell lung cancer.

Another exemplary embodiment is a method of determining whether a subject having or suspected of having a cancer will be responsive to treatment with an antibody-drug conjugate, composition, or pharmaceutical composition (e.g., any of the exemplary antibody-drug conjugates, compositions, or pharmaceutical compositions disclosed herein) by providing a biological sample from the subject; contacting the sample with the antibody-drug conjugate; and detecting binding of the antibody-drug conjugate to cancer cells in the sample. In some embodiments, the cancer cells in the sample express a target antigen. In some embodiments, the cancer expresses the target antigen EphA2. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer. In some embodiments, the cancer is breast cancer, non-small cell lung cancer, pancreatic cancer, esophageal cancer, head and neck cancer, stomach cancer, bladder cancer, or colon cancer. In some embodiments, the cancer is breast cancer or non-small cell lung cancer. In some embodiments, the sample is a tissue biopsy sample, a blood sample, or a bone marrow sample.

Methods of producing the described ADC compounds and compositions are also disclosed. An exemplary embodiment is a method of producing an antibody-drug conjugate by reacting an antibody or antigen-binding fragment with a cleavable linker joined or covalently attached to a Bcl-xL inhibitor under conditions that allow conjugation.

### BRIEF DESCRIPTION OF THE DRAWINGS

FIG. 1 shows EBC-1 Growth kinetics of EphA2-DANAPA-L11C-P25 ADC (30 mg/kg, SD, IV), 3207-DANAPA-L11C-P25 isotype control ADC (30 mg/kg, SD, IV), and EphA2-DANAPA CysMab control antibody (3.75 mg/kg, SD, IV) alone or in combination with paclitaxel (12.5 mg/kg, SD, IV).

FIG. 2 shows EBC-1 Growth kinetics of different dosages of EphA2-DANAPA-L11C-P25 ADC alone or in combination with paclitaxel.

FIG. 3 shows Panc03.27 Growth kinetics of EphA2-DANAPA-L11C-P25 ADC, 3207-DANAPA-L11C-P25 isotype control ADC, and EphA2-DANAPA CysMab control antibody alone or in combination with gemcitabine.

FIG. 4 shows Panc03.27 Growth kinetics of EphA2-DANAPA-L11C-P25 ADC, 3207-DANAPA-L11C-P25 isotype control ADC, and EphA2-DANAPA CysMab control antibody alone or in combination with MAPK inhibitors LXH254 and CFF272.

FIG. 5 shows binding affinity of EphA2 antibodies to HKB-11 cell line that has been transduced to overexpress EphA2 (Round 1)

FIG. 6 shows binding affinity of EphA2 antibodies to HKB-11 cell line that has been transduced to overexpress EphA2 (Round 2)

FIG. 7 shows binding affinity of EphA2 antibodies to HKB-11 cell line that has been transduced to overexpress EphA2 (Round 3)

FIG. 8 shows binding kinetics of human EphA2 expressed on HKB11 cells for the anti-EphA2 IgG antibody 1C₁ and its light chain point mutation IgGs.

FIGS. 9A and 9B show binding kinetics of human, mouse and cyno EphA2 ectodomain to the anti-EphA2 IgG antibody 1C₁ and its light chain point mutation IgGs.

### DETAILED DESCRIPTION OF ILLUSTRATIVE EMBODIMENTS

The disclosed compositions and methods may be understood more readily by reference to the following detailed description taken in connection with the accompanying figures, which form a part of this disclosure.

Throughout this text, the descriptions refer to compositions and methods of using the compositions. Where the disclosure describes or claims a feature or embodiment associated with a composition, such a feature or embodiment is equally applicable to the methods of using the composition. Likewise, where the disclosure describes or claims a feature or embodiment associated with a method of using a composition, such a feature or embodiment is equally applicable to the composition.

When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise. All references cited herein are incorporated by reference for any purpose. Where a reference and the specification conflict, the specification will control.

Unless the context of a description indicates otherwise, e.g., in the absence of symbols indicating specific point(s) of connectivity, when a structure or fragment of a structure is drawn, it may be used on its own or attached to other components of an ADC, and it may do so with any orientation, e.g., with the antibody attached at any suitable attachment point to a chemical moiety such as a linker-drug. Where indicated, however, components of an ADC are attached in the orientation shown in a given formula. For example, if Formula (1) is described as Ab-(L-D)ₚ and the group “-(L-D)” is described as

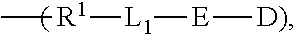

then the elaborated structure of Formula (1) is

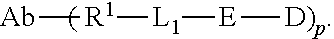

### It is not

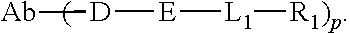

It is to be appreciated that certain features of the disclosed compositions and methods, which are, for clarity, described herein in the context of separate embodiments, may also be provided in combination in a single embodiment. Conversely, various features of the disclosed compositions and methods that are, for brevity, described in the context of a single embodiment, may also be provided separately or in any sub-combination.

As used throughout this application, antibody drug conjugates can be identified using a naming convention in the general format of “target antigen/antibody-linker-payload”. For example only, if an antibody drug conjugate is referred to as “Target X-L0-P0”, such a conjugate would comprise an antibody that binds Target X, a linker designated as L0, and a payload designated as P0. Alternatively, if an antibody drug conjugate is referred to as “anti-Target X-L0-P0”, such a conjugate would comprise an antibody that binds Target X, a linker designated as L0, and a payload designated as P0. In another alternative, if an antibody drug conjugate is referred to as “AbX-L0-P0”, such a conjugate would comprise the antibody designated as AbX, a linker designated as L0, and a payload designated as P0. A control antibody drug conjugate comprising a non-specific, isotype control antibody may be referenced as “isotype control IgG1-L0-P0” or “IgG1-L0-P0”.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen, carbon, nitrogen, oxygen, fluorine, and chlorine, such as ³H, ¹¹C, ¹³C, ¹⁴C, ¹⁵N, ¹⁸F, and ³⁶Cl. Accordingly, it should be understood that the present disclosure includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds can generally be prepared by conventional techniques known to those skilled in the art, e.g., using an appropriate isotopically-labeled reagents in place of the non-labeled reagent previously employed.

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include plural forms unless the context clearly dictates otherwise. The terms “comprising”, “having”, “being of” as in “being of a chemical formula”, “including”, and “containing” are to be construed as open terms (i.e., meaning “including but not limited to”) unless otherwise noted. Additionally whenever “comprising” or another open-ended term is used in an embodiment, it is to be understood that the same embodiment can be more narrowly claimed using the intermediate term “consisting essentially of” or the closed term “consisting of”.

The term “about” or “approximately,” when used in the context of numerical values and ranges, refers to values or ranges that approximate or are close to the recited values or ranges such that the embodiment may perform as intended, as is apparent to the skilled person from the teachings contained herein. In some embodiments, about means plus or minus 20%, 15%, 10%, 5%, 1%, 0.5%, or 0.1% of a numerical amount. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

The terms “antibody-drug conjugate,” “antibody conjugate,” “conjugate,” “immunoconjugate,” and “ADC” are used interchangeably, and refer to one or more therapeutic compounds (e.g., a Bcl-xL inhibitor) that is linked to one or more antibodies or antigen-binding fragments. In some embodiments, the ADC is defined by the generic formula: Ab-(L-D)ₚ (Formula 1), wherein Ab=an antibody or antigen-binding fragment (e.g., an anti-EphA2 antibody or antigen-binding fragment thereof), L=a linker moiety, D=a drug moiety (e.g., a Bcl-xL inhibitor drug moiety), and p=the number of drug moieties per antibody or antigen-binding fragment. In ADCs comprising a Bcl-xL inhibitor drug moiety, “p” refers to the number of Bcl-xL inhibitor compounds linked to the antibody or antigen-binding fragment.

The term “antibody” is used in the broadest sense to refer to an immunoglobulin molecule that recognizes and specifically binds to a target, such as a protein, polypeptide, carbohydrate, polynucleotide, lipid, or combinations of the foregoing through at least one antigen recognition site within the variable region of the immunoglobulin molecule. An antibody can be polyclonal or monoclonal, multiple or single chain, or an intact immunoglobulin, and may be derived from natural sources or from recombinant sources. An “intact” antibody is a glycoprotein that typically comprises at least two heavy (H) chains and two light (L) chains inter-connected by disulfide bonds. Each heavy chain is comprised of a heavy chain variable region (abbreviated herein as VH) and a heavy chain constant region. The heavy chain constant region comprises three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g., IgG1, IgG2, IgG3, IgG4, IgA1 and IgA2), or subclass. An antibody can be an intact antibody or an antigen-binding fragment thereof.

In some embodiments, the antibody or antibody fragment disclosed herein include modified or engineered amino acid residues, e.g., one or more cysteine residues, as sites for conjugation to a drug moiety (Junutula J R, et al., Nat Biotechnol 2008, 26:925-932). In one embodiment, the disclosure provides a modified antibody or antibody fragment comprising a substitution of one or more amino acids with cysteine at the positions described herein. Sites for cysteine substitution are in the constant regions of the antibody or antibody fragment and are thus applicable to a variety of antibody or antibody fragment, and the sites are selected to provide stable and homogeneous conjugates. A modified antibody or fragment can have one, two or more cysteine substitutions, and these substitutions can be used in combination with other modification and conjugation methods as described herein. Methods for inserting cysteine at specific locations of an antibody are known in the art, see, e.g., Lyons et al., (1990) Protein Eng., 3:703-708, WO 2011/005481, WO2014/124316, WO 2015/138615. In certain embodiments, a modified antibody comprises a substitution of one or more amino acids with cysteine on its constant region selected from positions 117, 119, 121, 124, 139, 152, 153, 155, 157, 164, 169, 171, 174, 189, 191, 195, 197, 205, 207, 246, 258, 269, 274, 286, 288, 290, 292, 293, 320, 322, 326, 333, 334, 335, 337, 344, 355, 360, 375, 382, 390, 392, 398, 400 and 422 of a heavy chain of the antibody, and wherein the positions are numbered according to the EU system. In some embodiments a modified antibody or antibody fragment comprises a substitution of one or more amino acids with cysteine on its constant region selected from positions 107, 108, 109, 114, 129, 142, 143, 145, 152, 154, 156, 159, 161, 165, 168, 169, 170, 182, 183, 197, 199, and 203 of a light chain of the antibody or antibody fragment, wherein the positions are numbered according to the EU system, and wherein the light chain is a human kappa light chain. In certain embodiments a modified antibody or antibody fragment thereof comprises a combination of substitution of two or more amino acids with cysteine on its constant regions wherein the combinations comprise substitutions at positions 375 of an antibody heavy chain, position 152 of an antibody heavy chain, position 360 of an antibody heavy chain, or position 107 of an antibody light chain and wherein the positions are numbered according to the EU system. In certain embodiments a modified antibody or antibody fragment thereof comprises a substitution of one amino acid with cysteine on its constant regions wherein the substitution is position 375 of an antibody heavy chain, position 152 of an antibody heavy chain, position 360 of an antibody heavy chain, position 107 of an antibody light chain, position 165 of an antibody light chain or position 159 of an antibody light chain and wherein the positions are numbered according to the EU system, and wherein the light chain is a kappa chain. In particular embodiments a modified antibody or antibody fragment thereof comprises a combination of substitution of two amino acids with cysteine on its constant regions wherein the combinations comprise substitutions at positions 375 of an antibody heavy chain and position 152 of an antibody heavy chain, wherein the positions are numbered according to the EU system. In particular embodiments a modified antibody or antibody fragment thereof comprises a substitution of one amino acid with cysteine at position 360 of an antibody heavy chain, wherein the positions are numbered according to the EU system. In other particular embodiments a modified antibody or antibody fragment thereof comprises a substitution of one amino acid with cysteine at position 107 of an antibody light chain and wherein the positions are numbered according to the EU system, and wherein the light chain is a kappa chain.

The term “antibody fragment” or “antigen-binding fragment” or “functional antibody fragment,” as used herein, refers to at least one portion of an antibody that retains the ability to specifically interact with (e.g., by binding, steric hinderance, stabilizing/destabilizing, spatial distribution) an epitope of an antigen (e.g., EphA2). Antigen-binding fragments may also retain the ability to internalize into an antigen-expressing cell. In some embodiments, antigen-binding fragments also retain immune effector activity. The terms antibody, antibody fragment, antigen-binding fragment, and the like, are intended to embrace the use of binding domains from antibodies in the context of larger macromolecules such as ADCs. It has been shown that fragments of a full-length antibody can perform the antigen binding function of a full-length antibody. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen-binding fragment can also be incorporated into single domain antibodies, maxibodies, minibodies, nanobodies, intrabodies, diabodies, triabodies, tetrabodies, bispecific or multi-specific antibody constructs, ADCs, v-NAR and bis-scFv (see, e.g., Holliger and Hudson (2005) Nat Biotechnol. 23 (9):1126-36). Antigen-binding fragments can also be grafted into scaffolds based on polypeptides such as a fibronectin type III (Fn3)(see U.S. Pat. No. 6,703,199, which describes fibronectin polypeptide minibodies). The term “scFv” refers to a fusion protein comprising at least one antigen-binding fragment comprising a variable region of a light chain and at least one antigen-binding fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, an scFv may have the VL and VH variable regions in either order, e.g., with respect to the N-terminal and C-terminal ends of the polypeptide, the scFv may comprise VL-linker-VH or may comprise VH-linker-VL. Antigen-binding fragments are obtained using conventional techniques known to those of skill in the art, and the binding fragments are screened for utility (e.g., binding affinity, internalization) in the same manner as are intact antibodies. Antigen-binding fragments, for example, may be prepared by cleavage of the intact protein, e.g., by protease or chemical cleavage.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991) “Sequences of Proteins of Immunological Interest,” 5th Ed. Public Health Service, National Institutes of Health, Bethesda, MD (“Kabat” numbering scheme); Al-Lazikani et al. (1997) J Mol Biol. 273 (4):927-48 (“Chothia” numbering scheme); ImMunoGenTics (IMGT) numbering (Lefranc (2001) Nucleic Acids Res. 29 (1):207-9; Lefranc et al. (2003) Dev Comp Immunol. 27 (1):55-77)(“IMGT” numbering scheme); or a combination thereof. In a combined Kabat and Chothia numbering scheme for a given CDR region (for example, HC CDR1, HC CDR2, HC CDR3, LC CDR1, LC CDR2, or LC CDR3), in some embodiments, the CDRs correspond to the amino acid residues that are defined as part of the Kabat CDR, together with the amino acid residues that are defined as part of the Chothia CDR. As used herein, the CDRs defined according to the “Chothia” number scheme are also sometimes referred to as “hypervariable loops.”

In some embodiments, under Kabat, the CDR amino acid residues in the heavy chain variable domain (VH) are numbered 31-35 (HCDR1)(e.g., insertion(s) after position 35), 50-65 (HCDR2), and 95-102 (HCDR3); and the CDR amino acid residues in the light chain variable domain (VL) are numbered 24-34 (LCDR1)(e.g., insertion(s) after position 27), 50-56 (LCDR2), and 89-97 (LCDR3). In some embodiments, under Chothia, the CDR amino acids in the VH are numbered 26-32 (HCDR1)(e.g., insertion(s) after position 31), 52-56 (HCDR2), and 95-102 (HCDR3); and the amino acid residues in VL are numbered 26-32 (LCDR1)(e.g., insertion(s) after position 30), 50-52 (LCDR2), and 91-96 (LCDR3). By combining the CDR definitions of both Kabat and Chothia, in some embodiments, the CDRs comprise or consist of, e.g., amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. In some embodiments, under IMGT, the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3). In some embodiments, under IMGT, the CDR regions of an antibody may be determined using the program IMGT/DomainGap Align.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256:495, or may be made by recombinant DNA methods (see, e.g., U.S. Pat. No. 4,816,567). Monoclonal antibodies may also be isolated from phage antibody libraries using the techniques described in Clackson et al. (1991) Nature 352:624-8, and Marks et al. (1991) J Mol Biol. 222:581-97, for example. The term also includes preparations of antibody molecules of single molecular composition. A monoclonal antibody composition displays a single binding specificity and affinity for a particular epitope.

The monoclonal antibodies described herein can be non-human, human, or humanized. The term specifically includes “chimeric” antibodies, in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity.

The term “human antibody,” as used herein, refers an antibody produced by a human or an antibody having an amino acid sequence of an antibody produced by a human. The term includes antibodies having variable regions in which both the framework and CDR regions are derived from sequences of human origin. Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik et al. ((2000) J Mol Biol. 296 (1):57-86). The structures and locations of immunoglobulin variable domains, e.g., CDRs, may be defined using well known numbering schemes, e.g., the Kabat numbering scheme, the Chothia numbering scheme, or a combination of Kabat and Chothia, and/or ImMunoGenTics (IMGT) numbering. The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody,” as used herein, is not intended to include antibodies in which CDR sequences derived from the germline of another mammalian species, such as a mouse, have been grafted onto human framework sequences.

The term “recombinant human antibody,” as used herein, refers to a human antibody that is prepared, expressed, created, or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In some embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody,” as used herein, refers to antibodies wherein the amino acid sequence of the immunoglobulin molecule is derived from two or more species. In some instances, the variable regions of both heavy and light chains correspond to the variable regions of antibodies derived from one species with the desired specificity, affinity, and activity while the constant regions are homologous to antibodies derived from another species (e.g., human) to minimize an immune response in the latter species.

As used herein, the term “humanized antibody” refers to forms of antibodies that contain sequences from non-human (e.g., murine) antibodies as well as human antibodies. Such antibodies are a type of chimeric antibody which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin. The humanized antibody can be further modified by the substitution of residues, either in the Fv framework region and/or within the replaced non-human residues to refine and optimize antibody specificity, affinity, and/or activity.

The term “Fc region,” as used herein, refers to a polypeptide comprising the CH3, CH2 and at least a portion of the hinge region of a constant domain of an antibody. Optionally, an Fc region may include a CH4 domain, present in some antibody classes. An Fc region may comprise the entire hinge region of a constant domain of an antibody. In some embodiments, an antibody or antigen-binding fragment comprises an Fc region and a CH1 region of an antibody. In some embodiments, an antibody or antigen-binding fragment comprises an Fc region CH3 region of an antibody. In some embodiments, an antibody or antigen-binding fragment comprises an Fc region, a CH1 region, and a kappa/lambda region from the constant domain of an antibody. In some embodiments, an antibody or antigen-binding fragment comprises a constant region, e.g., a heavy chain constant region and/or a light chain constant region. In some embodiments, such a constant region is modified compared to a wild-type constant region. That is, the polypeptide may comprise alterations or modifications to one or more of the three heavy chain constant domains (CH1, CH2, or CH3) and/or to the light chain constant region domain (CL). Example modifications include additions, deletions, or substitutions of one or more amino acids in one or more domains. Such changes may be included to optimize effector function, half-life, etc.

“Internalizing” as used herein in reference to an antibody or antigen-binding fragment refers to an antibody or antigen-binding fragment that is capable of being taken through the cell's lipid bilayer membrane to an internal compartment (i.e., “internalized”) upon binding to the cell, preferably into a degradative compartment in the cell. For example, an internalizing anti-EphA2 antibody is one that is capable of being taken into the cell after binding to EphA2 on the cell membrane. In some embodiments, the antibody or antigen-binding fragment used in the ADCs disclosed herein targets a cell surface antigen (e.g., EphA2) and is an internalizing antibody or internalizing antigen-binding fragment (i.e., the ADC transfers through the cellular membrane after antigen binding). In some embodiments, the internalizing antibody or antigen-binding fragment binds a receptor on the cell surface. An internalizing antibody or internalizing antigen-binding fragment that targets a receptor on the cell membrane may induce receptor-mediated endocytosis. In some embodiments, the internalizing antibody or internalizing antigen-binding fragment is taken into the cell via receptor-mediated endocytosis.

“Non-internalizing” as used herein in reference to an antibody or antigen-binding fragment refers to an antibody or antigen-binding fragment that remains at the cell surface upon binding to the cell. In some embodiments, the antibody or antigen-binding fragment used in the ADCs disclosed herein targets a cell surface antigen and is a non-internalizing antibody or non-internalizing antigen-binding fragment (i.e., the ADC remains at the cell surface and does not transfer through the cellular membrane after antigen binding). In some embodiments, the non-internalizing antibody or antigen-binding fragment binds a non-internalizing receptor or other cell surface antigen.

The term “EPH receptor A2,” “ephrin type-A receptor 2,” or “EphA2” as used herein, refers to any native form of human EphA2. The term encompasses full-length human EphA2 (e.g., NCBI Reference Sequence: NP_004422.2; SEQ ID NO: 61), as well as any form of human EphA2 that may result from cellular processing. The term also encompasses functional variants or fragments of human EphA2, including but not limited to splice variants, allelic variants, and isoforms that retain one or more biologic functions of human EphA2 (i.e., variants and fragments are encompassed unless the context indicates that the term is used to refer to the wild-type protein only). EphA2 can be isolated from human, or may be produced recombinantly or by synthetic methods.

The term “anti-EphA2 antibody” or “antibody that binds to EphA2,” as used herein, refers to any form of antibody or antigen-binding fragment thereof that binds, e.g., specifically binds, to EphA2. The term encompasses monoclonal antibodies (including full-length monoclonal antibodies), polyclonal antibodies, and biologically functional antigen-binding fragments so long as they bind, e.g., specifically bind, to EphA2. WO2007/030642 provides and is incorporated herein by reference for exemplary EphA2-binding sequences, including exemplary anti-EphA2 antibody sequences. In some embodiments, the anti-EphA2 antibody used in the ADCs disclosed herein is an internalizing antibody or internalizing antigen-binding fragment. 1C₁ (WO2007/030642) is an exemplary anti-EphA2 antibody.

The term “binding specificity,” as used herein, refers to the ability of an individual antibody or antigen binding fragment to preferentially react with one antigenic determinant over a different antigenic determinant. The degree of specificity indicates the extent to which an antibody or fragment preferentially binds to one antigenic determinant over a different antigenic determinant. Also, as used herein, the term “specific,” “specifically binds,” and “binds specifically” refers to a binding reaction between an antibody or antigen-binding fragment (e.g., an anti-EphA2 antibody) and a target antigen (e.g., EphA2) in a heterogeneous population of proteins and other biologics. Antibodies can be tested for specificity of binding by comparing binding to an appropriate antigen to binding to an irrelevant antigen or antigen mixture under a given set of conditions. If the antibody binds to the appropriate antigen with at least 2, 5, 7, 10 or more times more affinity than to the irrelevant antigen or antigen mixture, then it is considered to be specific. A “specific antibody” or a “target-specific antibody” is one that only binds the target antigen (e.g., EphA2), but does not bind (or exhibits minimal binding) to other antigens. In some embodiments, an antibody or antigen-binding fragment that specifically binds a target antigen (e.g., EphA2) has a K_{D} of less than 1×10⁻⁶ M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In some embodiments, the K_{D} is 1 pM to 500 pM. In some embodiments, the Ko is between 500 pM to 1 μM, 1 μM to 100 nM, or 100 mM to 10 nM.

The term “affinity,” as used herein, refers to the strength of interaction between antibody and antigen at single antigenic sites. Without being bound by theory, within each antigen binding site, the variable region of the antibody “arm” interacts through weak non-covalent forces with the antigen at numerous sites; the more interactions, typically the stronger the affinity. The binding affinity of an antibody is the sum of the attractive and repulsive forces operating between the antigenic determinant and the binding site of the antibody.

The term “Kₒₙ” or “Kₐ” refers to the on-rate constant for association of an antibody to the antigen to form the antibody/antigen complex. The rate can be determined using standard assays, such as a surface plasmon resonance, biolayer inferometry, or ELISA assay.

The term “K_{off}” or “K_{d}” refers to the off-rate constant for dissociation of an antibody from the antibody/antigen complex. The rate can be determined using standard assays, such as a surface plasmon resonance, biolayer inferometry, or ELISA assay.

The term “K_{D}” refers to the equilibrium dissociation constant of a particular antibody-antigen interaction. K_{D} is calculated by Kₐ/k_{d}. The rate can be determined using standard assays, such as a surface plasmon resonance, biolayer inferometry, or ELISA assay.

The term “epitope” refers to the portion of an antigen capable of being recognized and specifically bound by an antibody (or antigen-binding fragment). Epitope determinants generally consist of chemically active surface groupings of molecules such as amino acids or carbohydrate or sugar side chains and can have specific three-dimensional structural characteristics, as well as specific charge characteristics. When the antigen is a polypeptide, epitopes can be formed from contiguous amino acids or noncontiguous amino acids juxtaposed by tertiary folding of the polypeptide. An epitope may be “linear” or “conformational.” Conformational and linear epitopes are distinguished in that the binding to the former but not the latter is lost in the presence of denaturing solvents. The epitope bound by an antibody (or antigen-binding fragment) may be identified using any epitope mapping technique known in the art, including X-ray crystallography for epitope identification by direct visualization of the antigen-antibody complex, as well as monitoring the binding of the antibody to fragments or mutated variations of the antigen, or monitoring solvent accessibility of different parts of the antibody and the antigen. Exemplary strategies used to map antibody epitopes include, but are not limited to, array-based oligo-peptide scanning, limited proteolysis, site-directed mutagenesis, high-throughput mutagenesis mapping, hydrogen-deuterium exchange, and mass spectrometry (see, e.g., Gershoni et al. (2007) BioDrugs 21:145-56; and Hager-Braun and Tomer (2005) Expert Rev Proteomics 2:745-56).

Competitive binding and epitope binning can also be used to determine antibodies sharing identical or overlapping epitopes. Competitive binding can be evaluated using a cross-blocking assay, such as the assay described in “Antibodies, A Laboratory Manual,” Cold Spring Harbor Laboratory, Harlow and Lane (1^{st} edition 1988, 2^{nd} edition 2014). In some embodiments, competitive binding is identified when a test antibody or binding protein reduces binding of a reference antibody or binding protein to a target antigen such as EphA2 (e.g., a binding protein comprising CDRs and/or variable domains selected from those identified in Tables C-D), by at least about 50% in the cross-blocking assay (e.g., 50%, 60%, 70%, 80%, 90%, 95%, 99%, 99.5%, or more, or any percentage in between), and/or vice versa. In some embodiments, competitive binding can be due to shared or similar (e.g., partially overlapping) epitopes, or due to steric hindrance where antibodies or binding proteins bind at nearby epitopes (see, e.g., Tzartos, Methods in Molecular Biology (Morris, ed. (1998) vol. 66, pp. 55-66)). In some embodiments, competitive binding can be used to sort groups of binding proteins that share similar epitopes. For example, binding proteins that compete for binding can be “binned” as a group of binding proteins that have overlapping or nearby epitopes, while those that do not compete are placed in a separate group of binding proteins that do not have overlapping or nearby epitopes.

As used herein, the terms “peptide,” “polypeptide,” and “protein” are used interchangeably to refer to a polymer of amino acid residues. The terms encompass amino acid polymers comprising two or more amino acids joined to each other by peptide bonds, amino acid polymers in which one or more amino acid residues is an artificial chemical mimetic of a corresponding naturally-occurring amino acid, as well as naturally-occurring amino acid polymers and non-naturally-occurring amino acid polymers. The terms include, for example, biologically active fragments, substantially homologous polypeptides, oligopeptides, homodimers, heterodimers, variants of polypeptides, modified polypeptides, derivatives, analogs, fusion proteins, among others. The terms also include natural peptides, recombinant peptides, synthetic peptides, or a combination thereof. Unless otherwise indicated, a particular polypeptide sequence also implicitly encompasses conservatively modified variants thereof.

A “recombinant” protein refers to a protein (e.g., an antibody) made using recombinant techniques, e.g., through the expression of a recombinant nucleic acid.

An “isolated” protein refers to a protein unaccompanied by at least some of the material with which it is normally associated in its natural state. For example, a naturally-occurring polynucleotide or polypeptide present in a living organism is not isolated, but the same polynucleotide or polypeptide separated from some or all of the coexisting materials in the living organism, is isolated. The definition includes the production of an antibody in a wide variety of organisms and/or host cells that are known in the art.

An “isolated antibody,” as used herein, is an antibody that has been identified and separated from one or more (e.g., the majority) of the components (by weight) of its source environment, e.g., from the components of a hybridoma cell culture or a different cell culture that was used for its production. In some embodiments, the separation is performed such that it sufficiently removes components that may otherwise interfere with the suitability of the antibody for the desired applications (e.g., for therapeutic use). Methods for preparing isolated antibodies are known in the art and include, without limitation, protein A chromatography, anion exchange chromatography, cation exchange chromatography, virus retentive filtration, and ultrafiltration.

As used herein, the term “variant” refers to a nucleic acid sequence or an amino acid sequence that differs from a reference nucleic acid sequence or amino acid sequence respectively, but retains one or more biological properties of the reference sequence. A variant may contain one or more amino acid substitutions, deletions, and/or insertions (or corresponding substitution, deletion, and/or insertion of codons) with respect to a reference sequence. Changes in a nucleic acid variant may not alter the amino acid sequence of a peptide encoded by the reference nucleic acid sequence, or may result in amino acid substitutions, additions, deletions, fusions, and/or truncations. In some embodiments, a nucleic acid variant disclosed herein encodes an identical amino acid sequence to that encoded by the unmodified nucleic acid or encodes a modified amino acid sequence that retains one or more functional properties of the unmodified amino acid sequence. Changes in the sequence of peptide variants are typically limited or conservative, so that the sequences of the unmodified peptide and the variant are closely similar overall and, in many regions, identical. In some embodiments, a peptide variant retains one or more functional properties of the unmodified peptide sequence. A variant and unmodified peptide can differ in amino acid sequence by one or more substitutions, additions, deletions in any combination.

A variant of a nucleic acid or peptide can be a naturally-occurring variant or a variant that is not known to occur naturally. Variants of nucleic acids and peptides may be made by mutagenesis techniques, by direct synthesis, or by other techniques known in the art. A variant does not necessarily require physical manipulation of the reference sequence. As long as a sequence contains a different nucleic acid or amino acid as compared to a reference sequence, it is considered a “variant” regardless of how it was synthesized. In some embodiments, a variant has high sequence identity (i.e., 60% nucleic acid or amino acid sequence identity or higher) as compared to a reference sequence. In some embodiments, a peptide variant encompasses polypeptides having amino acid substitutions, deletions, and/or insertions as long as the polypeptide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% amino acid sequence identity with a reference sequence, or with a corresponding segment (e.g., a functional fragment) of a reference sequence, e.g., those variants that also retain one or more functions of the reference sequence. In some embodiments, a nucleic acid variant encompasses polynucleotides having amino acid substitutions, deletions, and/or insertions as long as the polynucleotide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% nucleic acid sequence identity with a reference sequence, or with a corresponding segment (e.g., a functional fragment) of a reference sequence.

The term “conservatively modified variant” applies to both amino acid and nucleic acid sequences. For nucleic acid sequences, conservatively modified variants refer to those nucleic acids which encode identical or essentially identical amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence. For polypeptide sequences, conservatively modified variants include individual substitutions, deletions, or additions to a polypeptide sequence which result in the substitution of an amino acid with a chemically similar amino acid. Conservative substitutions providing functionally similar amino acids are well known in the art.

The term “conservative sequence modifications,” as used herein, refers to amino acid modifications that do not significantly affect or alter the binding characteristics of, e.g., an antibody or antigen-binding fragment containing the amino acid sequence. Such conservative modifications include amino acid substitutions, additions, and deletions. Modifications can be introduced into an antibody or antigen-binding fragment by standard techniques known in the art, such as, e.g., site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, in some embodiments, one or more amino acid residues within an antibody can be replaced with other amino acid residues from the same side chain family and the altered antibody can be tested using the functional assays described herein.

The term “homologous” or “identity,” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions. For example, if half (e.g., five positions in a polymer ten subunits in length) of the positions in two sequences are matched or homologous, the two sequences are 50% homologous; if 90% of the positions (e.g., 9 of 10), are matched or homologous, the two sequences are 90% homologous.

Percentage of “sequence identity” can be determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence. The percent identity between the two sequences is a function of the number of identical positions shared by the sequences, taking into account the number of gaps, and the length of each gap, which need to be introduced for optimal alignment of the two sequences. Generally, the amino acid identity or homology between proteins disclosed herein and variants thereof, including variants of target antigens (such as EphA2) and variants of antibody variable domains (including individual variant CDRs), is at least 80% to the sequences depicted herein, e.g., identities or homologies of at least 85%, 90%, 91%, 92%, 93%, 94%, 95%, 96%, 97%, 98%, 99%, almost 100%, or 100%.

The comparison of sequences and determination of percent identity between two sequences can be accomplished using a mathematical algorithm. In some embodiments, the percent identity between two amino acid sequences is determined using the Needleman and Wunsch ((1970) J Mol Biol. 48:444-53) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In some embodiments, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. An exemplary set of parameters is a Blossum 62 scoring matrix with a gap penalty of 12, a gap extend penalty of 4, and a frameshift gap penalty of 5. The percent identity between two amino acid or nucleotide sequences can also be determined using the algorithm of Meyers and Miller ((1989) CABIOS 4:11-17) which has been incorporated into the ALIGN program (version 2.0), using a PAM120 weight residue table, a gap length penalty of 12 and a gap penalty of 4.

The term “agent” is used herein to refer to a chemical compound, a mixture of chemical compounds, a biological macromolecule, an extract made from biological materials, or a combination of two or more thereof. The term “therapeutic agent” or “drug” refers to an agent that is capable of modulating a biological process and/or has biological activity. The Bcl-xL inhibitors and the ADCs comprising them, as described herein, are exemplary therapeutic agents.

The term “chemotherapeutic agent” or “anti-cancer agent” is used herein to refer to all agents that are effective in treating cancer (regardless of mechanism of action). Inhibition of metastasis or angiogenesis is frequently a property of a chemotherapeutic agent.

Chemotherapeutic agents include antibodies, biological molecules, and small molecules, and encompass the Bcl-xL inhibitors and ADCs comprising them, as described herein. A chemotherapeutic agent may be a cytotoxic or cytostatic agent. The term “cytostatic agent” refers to an agent that inhibits or suppresses cell growth and/or multiplication of cells. The term “cytotoxic agent” refers to a substance that causes cell death primarily by interfering with a cell's expression activity and/or functioning.

The term “B-cell lymphoma-extra large” or “Bcl-xL,” as used herein, refers to any native form of human Bcl-xL, an anti-apoptotic member of the Bcl-2 protein family. The term encompasses full-length human Bcl-xL (e.g., UniProt Reference Sequence: Q07817-1), as well as any form of human Bcl-xL that may result from cellular processing. The term also encompasses functional variants or fragments of human Bcl-xL, including but not limited to splice variants, allelic variants, and isoforms that retain one or more biologic functions of human Bcl-xL (i.e., variants and fragments are encompassed unless the context indicates that the term is used to refer to the wild-type protein only). Bcl-xL can be isolated from human, or may be produced recombinantly or by synthetic methods.

The term “inhibit” or “inhibition” or “inhibiting,” as used herein, means to reduce a biological activity or process by a measurable amount, and can include but does not require complete prevention or inhibition. In some embodiments, “inhibition” means to reduce the expression and/or activity of Bcl-xL and/or one or more upstream modulators or downstream targets thereof.

The term “Bcl-xL inhibitor,” as used herein, refers to an agent capable of reducing the expression and/or activity of Bcl-xL and/or one or more upstream modulators or downstream targets thereof. Exemplary Bcl-xL modulators (including exemplary inhibitors of Bcl-xL) are described in WO2021/018858, WO2021/018857, WO2010/080503, WO2010/080478, WO2013/055897, WO2013/055895, WO2016/094509, WO2016/094517, WO2016/094505, Tao et al., ACS Medicinal Chemistry Letters (2014), 5 (10), 1088-109, and Wang et al., ACS Medicinal Chemistry Letters (2020), 11 (10), 1829-1836, each of which are incorporated herein by reference as exemplary Bcl-xL modulators, including exemplary Bcl-xL inhibitors, that can be included as drug moieties in the disclosed ADCs.

As used herein, a “Bcl-xL inhibitor drug moiety”, “Bcl-xL inhibitor”, and the like refer to the component of an ADC or composition that provides the structure of a Bcl-xL inhibitor compound or a compound modified for attachment to an ADC that retains essentially the same, similar, or enhanced biological function or activity as compared to the original compound. In some embodiments, Bcl-xL inhibitor drug moiety is component (D) in an ADC of Formula (1).

The term “cancer,” as used herein, refers to the presence of cells possessing characteristics typical of cancer-causing cells, such as uncontrolled proliferation, immortality, metastatic potential, rapid growth and proliferation rate, and/or certain morphological features. Often, cancer cells can be in the form of a tumor or mass, but such cells may exist alone within a subject, or may circulate in the blood stream as independent cells, such as leukemic or lymphoma cells. The term “cancer” includes all types of cancers and cancer metastases, including hematological cancers, solid tumors, sarcomas, carcinomas and other solid and non-solid tumor cancers. Hematological cancers may include B-cell malignancies, cancers of the blood (leukemias), cancers of plasma cells (myelomas, e.g., multiple myeloma), or cancers of the lymph nodes (lymphomas). Exemplary B-cell malignancies include chronic lymphocytic leukemia (CLL), follicular lymphoma, mantle cell lymphoma, and diffuse large B-cell lymphoma. Leukemias may include acute lymphoblastic leukemia (ALL), acute myeloid leukemia (AML), chronic lymphocytic leukemia (CLL), chronic myelogenous leukemia (CML), chronic myelomonocytic leukemia (CMML), acute monocytic leukemia (AMOL), etc. The terms “acute lymphoblastic leukemia” and “acute lymphocytic leukemia” can be used interchangeably to describe ALL. Lymphomas may include Hodgkin's lymphoma, non-Hodgkin's lymphoma, etc. Other hematologic cancers may include myelodysplasia syndrome (MDS). Solid tumors may include carcinomas such as adenocarcinoma, e.g., breast cancer, pancreatic cancer, prostate cancer, colon or colorectal cancer, lung cancer, gastric cancer, cervical cancer, endometrial cancer, ovarian cancer, cholangiocarcinoma, glioma, melanoma, etc. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, or head and neck cancer. In some embodiments, the cancer is a lymphoma or gastric cancer.

As used herein, the term “tumor” refers to any mass of tissue that results from excessive cell growth or proliferation, either benign or malignant, including precancerous lesions. In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, or spleen cancer. In some embodiments, the tumor is a gastric cancer.

The terms “tumor cell” and “cancer cell” may be used interchangeably herein and refer to individual cells or the total population of cells derived from a tumor or cancer, including both non-tumorigenic cells and cancer stem cells. The terms “tumor cell” and “cancer cell” will be modified by the term “non-tumorigenic” when referring solely to those cells lacking the capacity to renew and differentiate to distinguish those cells from cancer stem cells.

The term “target-negative,” “target antigen-negative,” or “antigen-negative,” as used herein, refers to the absence of target antigen expression by a cell or tissue. The term “target-positive,” “target antigen-positive,” or “antigen-positive” refers to the presence of target antigen expression. For example, a cell or a cell line that does not express a target antigen may be described as target-negative, whereas a cell or cell line that expresses a target antigen may be described as target-positive.

The terms “subject” and “patient” are used interchangeably herein to refer to any human or non-human animal in need of treatment. Non-human animals include all vertebrates (e.g., mammals and non-mammals) such as any mammal. Non-limiting examples of mammals include humans, chimpanzees, apes, monkeys, cattle, horses, sheep, goats, swine, rabbits, dogs, cats, rats, mice, and guinea pigs. Non-limiting examples of non-mammals include birds and fish. In some embodiments, the subject is a human.

The term “a subject in need of treatment,” as used herein, refers to a subject that would benefit biologically, medically, or in quality of life from a treatment (e.g., a treatment with any one or more of the exemplary ADC compounds described herein).

As used herein, the term “treat,” “treating,” or “treatment” refers to any improvement of any consequence of disease, disorder, or condition, such as prolonged survival, less morbidity, and/or a lessening of side effects which result from an alternative therapeutic modality. In some embodiments, treatment comprises delaying or ameliorating a disease, disorder, or condition (i.e., slowing or arresting or reducing the development of a disease or at least one of the clinical symptoms thereof). In some embodiments, treatment comprises delaying, alleviating, or ameliorating at least one physical parameter of a disease, disorder, or condition, including those which may not be discernible by the patient. In some embodiments, treatment comprises modulating a disease, disorder, or condition, either physically (e.g., stabilization of a discernible symptom), physiologically (e.g., stabilization of a physical parameter), or both. In some embodiments, treatment comprises administration of a described ADC compound or composition to a subject, e.g., a patient, to obtain a treatment benefit enumerated herein. The treatment can be to cure, heal, alleviate, delay, prevent, relieve, alter, remedy, ameliorate, palliate, improve, or affect a disease, disorder, or condition (e.g., a cancer), the symptoms of a disease, disorder, or condition (e.g., a cancer), or a predisposition toward a disease, disorder, or condition (e.g., a cancer). In some embodiments, in addition to treating a subject having a disease, disorder, or condition, a composition disclosed herein can also be provided prophylactically to prevent or reduce the likelihood of developing that disease, disorder, or condition.

As used herein, the term “prevent”, “preventing,” or “prevention” of a disease, disorder, or condition refers to the prophylactic treatment of the disease, disorder, or condition; or delaying the onset or progression of the disease, disorder, or condition.

As used herein, a “pharmaceutical composition” refers to a preparation of a composition, e.g., an ADC compound or composition, in addition to at least one other (and optionally more than one other) component suitable for administration to a subject, such as a pharmaceutically acceptable carrier, stabilizer, diluent, dispersing agent, suspending agent, thickening agent, and/or excipient. The pharmaceutical compositions provided herein are in such form as to permit administration and subsequently provide the intended biological activity of the active ingredient(s) and/or to achieve a therapeutic effect. The pharmaceutical compositions provided herein preferably contain no additional components which are unacceptably toxic to a subject to which the formulation would be administered.

As used herein, the terms “pharmaceutically acceptable carrier” and “physiologically acceptable carrier,” which may be used interchangeably, refer to a carrier or a diluent that does not cause significant irritation to a subject and does not abrogate the biological activity and properties of the administered ADC compound or composition and/or any additional therapeutic agent in the composition. Pharmaceutically acceptable carriers may enhance or stabilize the composition or can be used to facilitate preparation of the composition. Pharmaceutically acceptable carriers can include solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. The carrier may be selected to minimize adverse side effects in the subject, and/or to minimize degradation of the active ingredient(s). An adjuvant may also be included in any of these formulations.

As used herein, the term “excipient” refers to an inert substance added to a pharmaceutical composition to further facilitate administration of an active ingredient. Formulations for parenteral administration can, for example, contain excipients such as sterile water or saline, polyalkylene glycols such as polyethylene glycol, vegetable oils, or hydrogenated napthalenes. Other exemplary excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20.

The term “pharmaceutically acceptable salt,” as used herein, refers to a salt which does not abrogate the biological activity and properties of the compounds of the invention, and does not cause significant irritation to a subject to which it is administered. Examples of such salts include, but are not limited to: (a) acid addition salts formed with inorganic acids, for example, hydrochloric acid, hydrobromic acid, sulfuric acid, phosphoric acid, nitric acid and the like; and salts formed with organic acids, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine. See, e.g., Haynes et al., “Commentary: Occurrence of Pharmaceutically Acceptable Anions and Cations in the Cambridge Structural Database,” J. Pharmaceutical Sciences, vol. 94, no. 10 (2005), and Berge et al., “Pharmaceutical Salts,” J. Pharmaceutical Sciences, vol. 66, no. 1 (1977), which are incorporated by reference herein.

In some embodiments, depending on their electronic charge, the antibody-drug conjugates (ADCs), linkers, payloads and linker-payloads described herein can contain a monovalent anionic counterion M₁⁻. Any suitable anionic counterion can be used. In certain embodiments, the monovalent anionic counterion is a pharmaceutically acceptable monovalent anionic counterion. In certain embodiments, the monovalent anionic counterion M₁⁻ can be selected from bromide, chloride, iodide, acetate, trifluoroacetate, benzoate, mesylate, tosylate, triflate, formate, or the like. In some embodiments, the monovalent anionic counterion M₁⁻ is trifluoroacetate or formate.

As used herein, the term “therapeutically effective amount” or “therapeutically effective dose,” refers to an amount of a compound described herein, e.g., an ADC compound or composition described herein, to effect the desired therapeutic result (i.e., reduction or inhibition of an enzyme or a protein activity, amelioration of symptoms, alleviation of symptoms or conditions, delay of disease progression, a reduction in tumor size, inhibition of tumor growth, prevention of metastasis). In some embodiments, a therapeutically effective amount does not induce or cause undesirable side effects. In some embodiments, a therapeutically effective amount induces or causes side effects but only those that are acceptable by a treating clinician in view of a patient's condition. In some embodiments, a therapeutically effective amount is effective for detectable killing, reduction, and/or inhibition of the growth or spread of cancer cells, the size or number of tumors, and/or other measure of the level, stage, progression and/or severity of a cancer. The term also applies to a dose that will induce a particular response in target cells, e.g., a reduction, slowing, or inhibition of cell growth. A therapeutically effective amount can be determined by first administering a low dose, and then incrementally increasing that dose until the desired effect is achieved. A therapeutically effective amount can also vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific amount may vary depending on, for example, the particular pharmaceutical composition, the subject and their age and existing health conditions or risk for health conditions, the dosing regimen to be followed, the severity of the disease, whether it is administered in combination with other agents, timing of administration, the tissue to which it is administered, and the physical delivery system in which it is carried. In the case of cancer, a therapeutically effective amount of an ADC may reduce the number of cancer cells, reduce tumor size, inhibit (e.g., slow or stop) tumor metastasis, inhibit (e.g., slow or stop) tumor growth, and/or relieve one or more symptoms.

As used herein, the term “prophylactically effective amount” or “prophylactically effective dose,” refers to an amount of a compound disclosed herein, e.g., an ADC compound or composition described herein, that is effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. In some embodiments, a prophylactically effective amount can prevent the onset of disease symptoms, including symptoms associated with a cancer.

The term “p” or “drug loading” or “drug: antibody ratio” or “drug-to-antibody ratio” or “DAR” refers to the number of drug moieties per antibody or antigen-binding fragment, i.e., drug loading, or the number of -L-D moieties per antibody or antigen-binding fragment (Ab) in ADCs of Formula (1). In ADCs comprising a Bcl-xL inhibitor drug moiety, “p” refers to the number of Bcl-xL inhibitor compounds linked to the antibody or antigen-binding fragment. For example, if two Bcl-xL inhibitor compounds are linked to an antibody or antigen-binding fragment, p=2. In compositions comprising multiple copies of ADCs of Formula (1), “average p” refers to the average number of -L-D moieties per antibody or antigen-binding fragment, also referred to as “average drug loading.”

### Antibody-Drug Conjugates

The antibody-drug conjugate (ADC) compounds of the present disclosure include those with anti-cancer activity. In particular, the ADC compounds include an antibody or antigen-binding fragment conjugated (i.e., covalently attached by a linker) to a drug moiety (e.g., a Bcl-xL inhibitor), wherein the drug moiety when not conjugated to an antibody or antigen-binding fragment has a cytotoxic or cytostatic effect. In some embodiments, the drug moiety when not conjugated to an antibody or antigen-binding fragment is capable of reducing the expression and/or activity of Bcl-xL and/or one or more upstream modulators or downstream targets thereof. Without being bound by theory, by targeting Bcl-xL expression and/or activity, in some embodiments, the ADCs disclosed herein may provide potent anti-cancer agents. Also, without being bound by theory, by conjugating the drug moiety to an antibody that binds an antigen associated with expression in a tumor cell or cancer, the ADC may provide improved activity, better cytotoxic specificity, and/or reduced off-target killing as compared to the drug moiety when administered alone.

In some embodiments, therefore, the components of the ADC are selected to (i) retain one or more therapeutic properties exhibited by the antibody and drug moieties in isolation, (ii) maintain the specific binding properties of the antibody or antigen-binding fragment; (iii) optimize drug loading and drug-to-antibody ratios; (iv) allow delivery, e.g., intracellular delivery, of the drug moiety via stable attachment to the antibody or antigen-binding fragment; (v) retain ADC stability as an intact conjugate until transport or delivery to a target site; (vi) minimize aggregation of the ADC prior to or after administration; (vii) allow for the therapeutic effect, e.g., cytotoxic effect, of the drug moiety after cleavage or other release mechanism in the cellular environment; (viii) exhibit in vivo anti-cancer treatment efficacy comparable to or superior to that of the antibody and drug moieties in isolation; (ix) minimize off-target killing by the drug moiety; and/or (x) exhibit desirable pharmacokinetic and pharmacodynamics properties, formulatability, and toxicologic/immunologic profiles. Each of these properties may provide for an improved ADC for therapeutic use (Ab et al. (2015) Mol Cancer Ther. 14:1605-13).

The ADC compounds of the present disclosure may selectively deliver an effective dose of a cytotoxic or cytostatic agent to cancer cells or to tumor tissue. In some embodiments, the cytotoxic and/or cytostatic activity of the ADC is dependent on target antigen expression in a cell. In some embodiments, the disclosed ADCs are particularly effective at killing cancer cells expressing a target antigen while minimizing off-target killing. In some embodiments, the disclosed ADCs do not exhibit a cytotoxic and/or cytostatic effect on cancer cells that do not express a target antigen.

Provided herein, in certain aspects, are ADC compounds comprising an anti-EphA2 antibody or antigen-binding fragment thereof (Ab), a Bcl-XL inhibitor drug moiety (D), and a linker moiety (L) that covalently attaches Ab to D. In some embodiments, provided herein, are ADC compounds comprising an antibody or antigen-binding fragment thereof (Ab) which targets a cancer cell, a Bcl-xL inhibitor drug moiety (D), and a linker moiety (L) that covalently attaches Ab to D. In some embodiments, the antibody or antigen-binding fragment is able to bind to a tumor-associated antigen (e.g., EphA2), e.g., with high specificity and high affinity. In some embodiments, the antibody or antigen-binding fragment is internalized into a target cell upon binding, e.g., into a degradative compartment in the cell. In some embodiments, the ADCs internalize upon binding to a target cell, undergo degradation, and release the Bcl-xL inhibitor drug moiety to kill cancer cells. The Bcl-xL inhibitor drug moiety may be released from the antibody and/or the linker moiety of the ADC by enzymatic action, hydrolysis, oxidation, or any other mechanism.

An exemplary ADC has Formula (1):

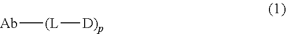

wherein Ab=an anti-EphA2 antibody or antigen-binding fragment, L=a linker moiety, D=a Bcl-xL inhibitor drug moiety, and p=the number of Bcl-xL inhibitor drug moieties per antibody or antigen-binding fragment.

### A. Antibodies

In some embodiment, the anti-EphA2 antibody or antigen-binding fragment (Ab) of Formula (1) specifically binds to a target antigen on a cell. In some embodiment, the anti-EphA2 antibody or antigen-binding fragment (Ab) of Formula (1) specifically binds to a target antigen on a cancer cell. In some embodiment, said cell or said cancer cell expresses EphA2. In some embodiments, the target antigen EphA2 has the following amino acid sequence:

<NCBI Reference Sequence: NP_004422.2>
(SEQ ID NO: 61)
MELQAARACFALLWGCALAAAAAAQGKEVVLLDFAAAGGELGWLTH
PYGKGWDLMQNIMNDMPIYMYSVCNVMSGDQDNWLRTNWVYRGEA
ERIFIELKFTVRDCNSFPGGASSCKETFNLYYAESDLDYGTNFQK
RLFTKIDTIAPDEITVSSDFEARHVKLNVEERSVGPLTRKGFYLA
FQDIGACVALLSVRVYYKKCPELLQGLAHFPETIAGSDAPSLATV
AGTCVDHAVVPPGGEEPRMHCAVDGEWLVPIGQCLCQAGYEKVED
ACQACSPGFFKFEASESPCLECPEHTLPSPEGATSCECEEGFFRA
PQDPASMPCTRPPSAPHYLTAVGMGAKVELRWTPPQDSGGREDIV
YSVTCEQCWPESGECGPCEASVRYSEPPHGLTRTSVTVSDLEPHM
NYTFTVEARNGVSGLVTSRSFRTASVSINQTEPPKVRLEGRSTTS
LSVSWSIPPPQQSRVWKYEVTYRKKGDSNSYNVRRTEGFSVTLDD
LAPDTTYLVQVQALTQEGQGAGSKVHEFQTLSPEGSGNLAVIGGV
AVGVVLLLVLAGVGFFIHRRRKNQRARQSPEDVYFSKSEQLKPLK
TYVDPHTYEDPNQAVLKFTTEIHPSCVTRQKVIGAGEFGEVYKGM
LKTSSGKKEVPVAIKTLKAGYTEKQRVDFLGEAGIMGQFSHHNII
RLEGVISKYKPMMIITEYMENGALDKFLREKDGEFSVLQLVGMLR
GIAAGMKYLANMNYVHRDLAARNILVNSNLVCKVSDFGLSRVLED
DPEATYTTSGGKIPIRWTAPEAISYRKFTSASDVWSFGIVMWEVM
TYGERPYWELSNHEVMKAINDGFRLPTPMDCPSAIYQLMMQCWQQ
ERARRPKFADIVSILDKLIRAPDSLKTLADFDPRVSIRLPSTSGS
EGVPFRTVSEWLESIKMQQYTEHFMAAGYTAIEKVVQMINDDIKR
IGVRLPGHQKRIAYSLLGLKDQVNTVGIPI

The anti-EphA2 antibody or antigen-binding fragment may bind to a target antigen with a dissociation constant (K_{D}) of ≤1 mM, ≤100 nM or ≤10 nM, or any amount in between, as measured by, e.g., BIAcore® analysis. In some embodiments, the K_{D} is 1 pM to 500 pM. In some embodiments, the Ko is between 500 pM to 1 μM, 1 μM to 100 nM, or 100 mM to 10 nM.

In some embodiments, the anti-EphA2 antibody or antigen-binding fragment is a four-chain anti-EphA2 antibody (also referred to as an immunoglobulin or a full-length or intact antibody), comprising two heavy chains and two light chains. In some embodiments, the anti-EphA2 antibody or antigen-binding fragment is an anti-EphA2 antigen-binding fragment of an immunoglobulin. In some embodiments, the anti-EphA2 antibody or antigen-binding fragment is an anti-EphA2 antigen-binding fragment of an immunoglobulin that retains the ability to bind a target cancer antigen and/or provide at least one function of the immunoglobulin.

In some embodiments, the anti-EphA2 antibody or antigen-binding fragment is an internalizing anti-EphA2 antibody or internalizing anti-EphA2 antigen-binding fragment thereof. In some embodiments, the internalizing anti-EphA2 antibody or internalizing anti-EphA2 antigen-binding fragment thereof binds to a target cancer antigen expressed on the surface of a cell and enters the cell upon binding. In some embodiments, the Bcl-xL inhibitor drug moiety of the ADC is released from the anti-EphA2 antibody or antigen-binding fragment of the ADC after the ADC enters and is present in a cell expressing the target cancer antigen (i.e., after the ADC has been internalized), e.g., by cleavage, by degradation of the antibody or antigen-binding fragment, or by any other suitable release mechanism. In some embodiment, said cancer expresses EphA2.

In some embodiments, the anti-EphA2 antibodies comprise mutations that mediate reduced or no antibody-dependent cellular cytotoxicity (ADCC) or complement-dependent cytotoxicity (CDC). In some embodiments, these mutations are known as Fc Silencing, Fc Silent, or Fc Silenced mutations. In some embodiments, amino acid residues L234 and L235 of the IgG1 constant region are substituted to A234 and A235 (also known as “LALA”). In some embodiments, amino acid residue N297 of the IgG1 constant region is substituted to A297 (also known as “N297A”). In some embodiments, amino acid residues D265 and P329 of the IgG1 constant region are substituted to A265 and A329 (also known as “DAPA”). Other antibody Fc silencing mutations may also be used. In some embodiments, the Fc silencing mutations are used in combination, for example D265A, N297A and P329A (also known as “DANAPA”).

Amino acid sequences of exemplary anti-EphA2 antibodies of the present disclosure, are set forth in Tables C and D.

As set forth herein, if modifications are made to the anti-EphA2 antibodies, they are further designated with that modification. For example if select amino acids in the anti-EphA2 antibody have been changed to cysteines (e.g. E152C, S375C according to EU numbering of the antibody heavy chain to facilitate conjugation to linker-drug moieties) they are designated as “CysMab”; or if the anti-EphA2 antibody has been modified with Fc silencing mutations D265A, N297A and P329A of the IgG1 constant region according to EU numbering, “DANAPA” is added to the antibody name. If the anti-EphA2 antibody is used in an antibody drug conjugate, they are named using the following format: Antibody designation-linker-payload.

TABLE C
Amino acid sequences of mAb CDRs and variable regions

| | SEQ ID | | |
|---|---|---|---|
| Ab | NO | IgG chain | Amino acid sequence |
| EphA2 | 1 | VH | EVQLLESGGGLVQPGGSLRLSCAASGFTFSHYMMAWV |
| (E152C | | | RQAPGKGLEWVSRIGPSGGPTHYADSVKGRFTISRDN |
| S375C) 1C1 | | | SKNTLYLQMNSLRAEDTAVYYCAGYDSGYDYVAVAGP |
| | | | AEYFQHWGQGTLVTVSS |
| HCDR1 | 2 | Chothia | GFTFSHY |
| HCDR2 | 3 | Chothia | GPSGGP |
| HCDR3 | 4 | Chothia | YDSGYDYVAVAGPAEYFQH |
| HCDR1 | 5 | Kabat | HYMMA |
| HCDR2 | 6 | Kabat | RIGPSGGPTHYADSVKG |
| HCDR3 | 4 | Kabat | YDSGYDYVAVAGPAEYFQH |
| HCDR1 | 7 | IMGT | GFTFSHYM |
| HCDR2 | 8 | IMGT | IGPSGGPT |
| HCDR3 | 9 | IMGT | AGYDSGYDYVAVAGPAEYFQH |
| HCDR1 | 10 | Combined | GFTFSHYMMA |
| HCDR2 | 6 | Combined | RIGPSGGPTHYADSVKG |
| HCDR3 | 4 | Combined | YDSGYDYVAVAGPAEYFQH |
| EphA2 | 1 | VH | EVQLLESGGGLVQPGGSLRLSCAASGFTFSHYMMAWV |
| (E152C | | | RQAPGKGLEWVSRIGPSGGPTHYADSVKGRFTISRDN |
| S375C) | | | |
| DANAPA 1C1 | | | SKNTLYLQMNSLRAEDTAVYYCAGYDSGYDYVAVAGP |
| | | | AEYFQHWGQGTLVTVSS |
| HCDR1 | 2 | Chothia | GFTFSHY |
| HCDR2 | 3 | Chothia | GPSGGP |
| HCDR3 | 4 | Chothia | YDSGYDYVAVAGPAEYFQH |
| HCDR1 | 5 | Kabat | HYMMA |
| HCDR2 | 6 | Kabat | RIGPSGGPTHYADSVKG |
| HCDR3 | 4 | Kabat | YDSGYDYVAVAGPAEYFQH |
| HCDR1 | 7 | IMGT | GFTFSHYM |
| HCDR2 | 8 | IMGT | IGPSGGPT |
| HCDR3 | 9 | IMGT | AGYDSGYDYVAVAGPAEYFQH |
| HCDR1 | 10 | Combined | GFTFSHYMMA |
| HCDR2 | 6 | Combined | RIGPSGGPTHYADSVKG |
| HCDR3 | 4 | Combined | YDSGYDYVAVAGPAEYFQH |
| WT Light | 11 | VL | DIQMTQSPSSLSASVGDRVTITCRASQSISTWLAWYQ |
| Chain | | | QKPGKAPKLLIYKASNLHTGVPSRFSGSGSGTEFSLT |
| | | | ISGLQPDDFATYYCQQYNSYSRTFGQGTKVEIK |
| LCDR1 | 12 | Chothia | SQSISTW |
| LCDR2 | 13 | Chothia | KAS |
| LCDR3 | 14 | Chothia | YNSYSR |
| LCDR1 | 15 | Kabat | RASQSISTWLA |
| LCDR2 | 16 | Kabat | KASNLHT |
| LCDR3 | 17 | Kabat | QQYNSYSRT |
| LCDR1 | 18 | IMGT | QSISTW |
| LCDR2 | 13 | IMGT | KAS |
| LCDR3 | 17 | IMGT | QQYNSYSRT |
| LCDR1 | 15 | Combined | RASQSISTWLA |
| LCDR2 | 16 | Combined | KASNLHT |
| LCDR3 | 17 | Combined | QQYNSYSRT |
| Light | 19 | VL (S10T) | DIQMTQSPSTLSASVGDRVTITCRASQSISTWLAWYQ |
| Chain S10T | | | QKPGKAPKLLIYKASNLHTGVPSRFSGSGSGTEFSLT |
| | | | ISGLQPDDFATYYCQQYNSYSRTFGQGTKVEIK |
| LCDR1 | 12 | Chothia | SQSISTW |
| LCDR2 | 13 | Chothia | KAS |
| LCDR3 | 14 | Chothia | YNSYSR |
| LCDR1 | 15 | Kabat | RASQSISTWLA |
| LCDR2 | 16 | Kabat | KASNLHT |
| LCDR3 | 17 | Kabat | QQYNSYSRT |
| LCDR1 | 18 | IMGT | QSISTW |
| LCDR2 | 13 | IMGT | KAS |
| LCDR3 | 17 | IMGT | QQYNSYSRT |
| LCDR1 | 15 | Combined | RASQSISTWLA |
| LCDR2 | 16 | Combined | KASNLHT |
| LCDR3 | 17 | Combined | QQYNSYSRT |
| Light | 20 | VL (S72T) | DIQMTQSPSSLSASVGDRVTITCRASQSISTWLAWYQ |
| Chain S72T | | | QKPGKAPKLLIYKASNLHTGVPSRFSGSGSGTEFTLT |
| | | | ISGLQPDDFATYYCQQYNSYSRTFGQGTKVEIK |
| LCDR1 | 12 | Chothia | SQSISTW |
| LCDR2 | 13 | Chothia | KAS |
| LCDR3 | 14 | Chothia | YNSYSR |
| LCDR1 | 15 | Kabat | RASQSISTWLA |
| LCDR2 | 16 | Kabat | KASNLHT |
| LCDR3 | 17 | Kabat | QQYNSYSRT |
| LCDR1 | 18 | IMGT | QSISTW |
| LCDR2 | 13 | IMGT | KAS |
| LCDR3 | 17 | IMGT | QQYNSYSRT |
| LCDR1 | 15 | Combined | RASQSISTWLA |
| LCDR2 | 16 | Combined | KASNLHT |
| LCDR3 | 17 | Combined | QQYNSYSRT |
| Light | 21 | VL (G77S) | DIQMTQSPSSLSASVGDRVTITCRASQSISTWLAWYQ |
| Chain G77S | | | QKPGKAPKLLIYKASNLHTGVPSRFSGSGSGTEFSLT |
| | | | ISSLQPDDFATYYCQQYNSYSRTFGQGTKVEIK |
| LCDR1 | 12 | Chothia | SQSISTW |
| LCDR2 | 13 | Chothia | KAS |
| LCDR3 | 14 | Chothia | YNSYSR |
| LCDR1 | 15 | Kabat | RASQSISTWLA |
| LCDR2 | 16 | Kabat | KASNLHT |
| LCDR3 | 17 | Kabat | QQYNSYSRT |
| LCDR1 | 18 | IMGT | QSISTW |
| LCDR2 | 13 | IMGT | KAS |
| LCDR3 | 17 | IMGT | QQYNSYSRT |
| LCDR1 | 15 | Combined | RASQSISTWLA |
| LCDR2 | 16 | Combined | KASNLHT |
| LCDR3 | 17 | Combined | QQYNSYSRT |
| Light | 22 | VL (LC | DIQMTQSPSTLSASVGDRVTITCRASQSISTWLAWYQ |
| Chain | | S10T_S72 | QKPGKAPKLLIYKASNLHTGVPSRFSGSGSGTEFTLT |
| S10T_S72T | | T_G77S) | ISSLQPDDFATYYCQQYNSYSRTFGQGTKVEIK |
| G77S | | | |
| LCDR1 | 12 | Chothia | SQSISTW |
| LCDR2 | 13 | Chothia | KAS |
| LCDR3 | 14 | Chothia | YNSYSR |
| LCDR1 | 15 | Kabat | RASQSISTWLA |
| LCDR2 | 16 | Kabat | KASNLHT |
| LCDR3 | 17 | Kabat | QQYNSYSRT |
| LCDR1 | 18 | IMGT | QSISTW |
| LCDR2 | 13 | IMGT | KAS |
| LCDR3 | 17 | IMGT | QQYNSYSRT |
| LCDR1 | 15 | Combined | RASQSISTWLA |
| LCDR2 | 16 | Combined | KASNLHT |
| LCDR3 | 17 | Combined | QQYNSYSRT |
| Light | 23 | VL (S93Q) | DIQMTQSPSSLSASVGDRVTITCRASQSISTWLAWYQ |
| Chain S93Q | | | QKPGKAPKLLIYKASNLHTGVPSRFSGSGSGTEFSLT |
| | | | ISGLQPDDFATYYCQQYNQYSRTFGQGTKVEIK |
| LCDR1 | 12 | Chothia | SQSISTW |
| LCDR2 | 13 | Chothia | KAS |
| LCDR3 | 70 | Chothia | YNQYSR |
| LCDR1 | 15 | Kabat | RASQSISTWLA |
| LCDR2 | 16 | Kabat | KASNLHT |
| LCDR3 | 24 | Kabat | QQYNQYSRT |
| LCDR1 | 18 | IMGT | QSISTW |
| LCDR2 | 13 | IMGT | KAS |
| LCDR3 | 24 | IMGT | QQYNQYSRT |
| LCDR1 | 15 | Combined | RASQSISTWLA |
| LCDR2 | 16 | Combined | KASNLHT |
| LCDR3 | 24 | Combined | QQYNQYSRT |
| Light | 25 | VL (S93V) | DIQMTQSPSSLSASVGDRVTITCRASQSISTWLAWYQ |
| Chain S93V | | | QKPGKAPKLLIYKASNLHTGVPSRFSGSGSGTEFSLT |
| | | | ISGLQPDDFATYYCQQYNVYSRTFGQGTKVEIK |
| LCDR1 | 12 | Chothia | SQSISTW |
| LCDR2 | 13 | Chothia | KAS |
| LCDR3 | 26 | Chothia | YNVYSR |
| LCDR1 | 15 | Kabat | RASQSISTWLA |
| LCDR2 | 16 | Kabat | KASNLHT |
| LCDR3 | 27 | Kabat | QQYNVYSRT |
| LCDR1 | 18 | IMGT | QSISTW |
| LCDR2 | 13 | IMGT | KAS |
| LCDR3 | 27 | IMGT | QQYNVYSRT |
| LCDR1 | 15 | Combined | RASQSISTWLA |
| LCDR2 | 16 | Combined | KASNLHT |
| LCDR3 | 27 | Combined | QQYNVYSRT |
| Light | 28 | VL (S93A) | DIQMTQSPSSLSASVGDRVTITCRASQSISTWLAWYQ |
| Chain S93A | | | QKPGKAPKLLIYKASNLHTGVPSRFSGSGSGTEFSLT |
| | | | ISGLQPDDFATYYCQQYNAYSRTFGQGTKVEIK |
| LCDR1 | 12 | Chothia | SQSISTW |
| LCDR2 | 13 | Chothia | KAS |
| LCDR3 | 29 | Chothia | YNAYSR |
| LCDR1 | 15 | Kabat | RASQSISTWLA |
| LCDR2 | 16 | Kabat | KASNLHT |
| LCDR3 | 30 | Kabat | QQYNAYSRT |
| LCDR1 | 18 | IMGT | QSISTW |
| LCDR2 | 13 | IMGT | KAS |
| LCDR3 | 30 | IMGT | QQYNAYSRT |
| LCDR1 | 15 | Combined | RASQSISTWLA |
| LCDR2 | 16 | Combined | KASNLHT |
| LCDR3 | 30 | Combined | QQYNAYSRT |
| Light | 31 | VL (N92A) | DIQMTQSPSSLSASVGDRVTITCRASQSISTWLAWYQ |
| Chain N92A | | | QKPGKAPKLLIYKASNLHTGVPSRFSGSGSGTEFSLT |
| | | | ISGLQPDDFATYYCQQYASYSRTFGQGTKVEIK |
| LCDR1 | 12 | Chothia | SQSISTW |
| LCDR2 | 13 | Chothia | KAS |
| LCDR3 | 32 | Chothia | YASYSR |
| LCDR1 | 15 | Kabat | RASQSISTWLA |
| LCDR2 | 16 | Kabat | KASNLHT |
| LCDR3 | 33 | Kabat | QQYASYSRT |
| LCDR1 | 18 | IMGT | QSISTW |
| LCDR2 | 13 | IMGT | KAS |
| LCDR3 | 33 | IMGT | QQYASYSRT |
| LCDR1 | 15 | Combined | RASQSISTWLA |
| LCDR2 | 16 | Combined | KASNLHT |
| LCDR3 | 33 | Combined | QQYASYSRT |
| Light | 34 | VL (N92Q) | DIQMTQSPSSLSASVGDRVTITCRASQSISTWLAWYQ |
| Chain N92Q | | | QKPGKAPKLLIYKASNLHTGVPSRFSGSGSGTEFSLT |
| | | | ISGLQPDDFATYYCQQYQSYSRTFGQGTKVEIK |
| LCDR1 | 12 | Chothia | SQSISTW |
| LCDR2 | 13 | Chothia | KAS |
| LCDR3 | 35 | Chothia | YQSYSR |
| LCDR1 | 15 | Kabat | RASQSISTWLA |
| LCDR2 | 16 | Kabat | KASNLHT |
| LCDR3 | 36 | Kabat | QQYQSYSRT |
| LCDR1 | 18 | IMGT | QSISTW |
| LCDR2 | 13 | IMGT | KAS |
| LCDR3 | 36 | IMGT | QQYQSYSRT |
| LCDR1 | 15 | Combined | RASQSISTWLA |
| LCDR2 | 16 | Combined | KASNLHT |
| LCDR3 | 36 | Combined | QQYQSYSRT |
| IgG (3207) | 62 | VH | QVQLQQSGPGLVKPSQTLSLTCAISGDSVSSNSAAWS |
| | | | WIRQSPGRGLEWLGRIYYRSKWYNDYAVSVKSRITIN |
| | | | PDTSKNQFSLQLNSVTPEDTAVYYCARLDHRYHEDTV |
| | | | YPGMDVWGQGTLVTVSS |
| HCDR1 | 63 | Combined | GDSVSSNSAAWS |
| HCDR2 | 64 | Combined | RIYYRSKWYNDYAVSVKS |
| HCDR3 | 65 | Combined | LDHRYHEDTVYPGMDV |
| IgG (3207) | 66 | VL | DIELTQPPSVSVAPGQTARISCSGDNLPAYTVTWYQQ |
| | | | KPGQAPVLVIYDDSDRPSGIPERFSGSNSGNTATLTI |
| | | | SGTQAEDEADYYCASWDPSSGVVFGGGTKLTVL |
| LCDR1 | 67 | Combined | SGDNLPAYTVT |
| LCDR2 | 68 | Combined | DDSDRPS |
| LCDR3 | 69 | Combined | ASWDPSSGVV |
| anti-EphA2 | 1 | VH | EVQLLESGGGLVQPGGSLRLSCAASGFTFSHYMMAWV |
| 1C1 hIgG1 | | | RQAPGKGLEWVSRIGPSGGPTHYADSVKGRFTISRDN |
| | | | SKNTLYLQMNSLRAEDTAVYYCAGYDSGYDYVAVAGP |
| | | | AEYFQHWGQGTLVTVSS |
| HCDR1 | 10 | Combined | GFTFSHYMMA |
| HCDR2 | 6 | Combined | RIGPSGGPTHYADSVKG |
| HCDR3 | 4 | Combined | YDSGYDYVAVAGPAEYFQH |
| HCDR1 | 2 | Chothia | GFTFSHY |
| HCDR2 | 3 | Chothia | GPSGGP |
| HCDR3 | 4 | Chothia | YDSGYDYVAVAGPAEYFQH |
| HCDR1 | 2 | Kabat | GFTFSHY |
| HCDR2 | 3 | Kabat | GPSGGP |
| HCDR3 | 4 | Kabat | YDSGYDYVAVAGPAEYFQH |
| HCDR1 | 7 | IMGT | GFTFSHYM |
| HCDR2 | 8 | IMGT | IGPSGGPT |
| HCDR3 | 9 | IMGT | AGYDSGYDYVAVAGPAEYFQH |
| anti-EphA2 | 11 | VL | DIQMTQSPSSLSASVGDRVTITCRASQSISTWLAWYQ |
| 1C1 hIgG1 | | | QKPGKAPKLLIYKASNLHTGVPSRFSGSGSGTEFSLT |
| | | | ISGLQPDDFATYYCQQYNSYSRTFGQGTKVEIK |
| LCDR1 | 15 | Combined | RASQSISTWLA |
| LCDR2 | 16 | Combined | KASNLHT |
| LCDR3 | 17 | Combined | QQYNSYSRT |
| LCDR1 | 12 | Chothia | SQSISTW |
| LCDR2 | 13 | Chothia | KAS |
| LCDR3 | 14 | Chothia | YNSYSR |
| LCDR1 | 12 | Kabat | SQSISTW |
| LCDR2 | 13 | Kabat | KAS |
| LCDR3 | 14 | Kabat | YNSYSR |
| LCDR1 | 18 | IMGT | QSISTW |
| LCDR2 | 13 | IMGT | KAS |
| LCDR3 | 17 | IMGT | QQYNSYSRT |
| Anti- | 1 | VH | EVQLLESGGGLVQPGGSLRLSCAASGFTFSHYMMAWV |
| EphA2_1C1 | | | RQAPGKGLEWVSRIGPSGGPTHYADSVKGRFTISRDN |
| DAR4_LC_N9 | | | SKNTLYLQMNSLRAEDTAVYYCAGYDSGYDYVAVAGP |
| 2Q_S72T | | | AEYFQHWGQGTLVTVSS |
| HCDR1 | 10 | Combined | GFTFSHYMMA |
| HCDR2 | 6 | Combined | RIGPSGGPTHYADSVKG |
| HCDR3 | 4 | Combined | YDSGYDYVAVAGPAEYFQH |
| HCDR1 | 2 | Chothia | GFTFSHY |
| HCDR2 | 3 | Chothia | GPSGGP |
| HCDR3 | 4 | Chothia | YDSGYDYVAVAGPAEYFQH |
| HCDR1 | 2 | Kabat | GFTFSHY |
| HCDR2 | 3 | Kabat | GPSGGP |
| HCDR3 | 4 | Kabat | YDSGYDYVAVAGPAEYFQH |
| HCDR1 | 7 | IMGT | GFTFSHYM |
| HCDR2 | 8 | IMGT | IGPSGGPT |
| HCDR3 | 9 | IMGT | AGYDSGYDYVAVAGPAEYFQH |
| Anti- | 71 | VL | DIQMTQSPSSLSASVGDRVTITCRASQSISTWLAWYQ |
| EphA2_1C1 | | | QKPGKAPKLLIYKASNLHTGVPSRFSGSGSGTEFTLT |
| DAR4_LC_N9 | | | ISGLQPDDFATYYCQQYQSYSRTFGQGTKVEIK |
| 2Q_S72T | | | |
| LCDR1 | 15 | Combined | RASQSISTWLA |
| LCDR2 | 16 | Combined | KASNLHT |
| LCDR3 | 36 | Combined | QQYQSYSRT |
| LCDR1 | 12 | Chothia | SQSISTW |
| LCDR2 | 13 | Chothia | KAS |
| LCDR3 | 35 | Chothia | YQSYSR |
| LCDR1 | 12 | Kabat | SQSISTW |
| LCDR2 | 13 | Kabat | KAS |
| LCDR3 | 35 | Kabat | YQSYSR |
| LCDR1 | 18 | IMGT | QSISTW |
| LCDR2 | 13 | IMGT | KAS |
| LCDR3 | 36 | IMGT | QQYQSYSRT |
| Anti- | 1 | VH | EVQLLESGGGLVQPGGSLRLSCAASGFTFSHYMMAWV |
| EphA2_1C1 | | | RQAPGKGLEWVSRIGPSGGPTHYADSVKGRFTISRDN |
| DAR4_LC_N9 | | | SKNTLYLQMNSLRAEDTAVYYCAGYDSGYDYVAVAGP |
| 2Q_G77S | | | AEYFQHWGQGTLVTVSS |
| HCDR1 | 10 | Combined | GFTFSHYMMA |
| HCDR2 | 6 | Combined | RIGPSGGPTHYADSVKG |
| HCDR3 | 4 | Combined | YDSGYDYVAVAGPAEYFQH |
| HCDR1 | 2 | Chothia | GFTFSHY |
| HCDR2 | 3 | Chothia | GPSGGP |
| HCDR3 | 4 | Chothia | YDSGYDYVAVAGPAEYFQH |
| HCDR1 | 2 | Kabat | GFTFSHY |
| HCDR2 | 3 | Kabat | GPSGGP |
| HCDR3 | 4 | Kabat | YDSGYDYVAVAGPAEYFQH |
| HCDR1 | 7 | IMGT | GFTFSHYM |
| HCDR2 | 8 | IMGT | IGPSGGPT |
| HCDR3 | 9 | IMGT | AGYDSGYDYVAVAGPAEYFQH |
| Anti- | 72 | VL | DIQMTQSPSSLSASVGDRVTITCRASQSISTWLAWYQ |
| EphA2_1C1 | | | QKPGKAPKLLIYKASNLHTGVPSRFSGSGSGTEFSLT |
| DAR4_LC_N9 | | | ISSLQPDDFATYYCQQYQSYSRTFGQGTKVEIK |
| 2Q_G77S | | | |
| LCDR1 | 15 | Combined | RASQSISTWLA |
| LCDR2 | 16 | Combined | KASNLHT |
| LCDR3 | 36 | Combined | QQYQSYSRT |
| LCDR1 | 12 | Chothia | SQSISTW |
| LCDR2 | 13 | Chothia | KAS |
| LCDR3 | 35 | Chothia | YQSYSR |
| LCDR1 | 12 | Kabat | SQSISTW |
| LCDR2 | 13 | Kabat | KAS |
| LCDR3 | 35 | Kabat | YQSYSR |
| LCDR1 | 18 | IMGT | QSISTW |
| LCDR2 | 13 | IMGT | KAS |
| LCDR3 | 36 | IMGT | QQYQSYSRT |
| Anti- | 1 | VH | EVQLLESGGGLVQPGGSLRLSCAASGFTFSHYMMAWV |
| EphA2_1C1 | | | RQAPGKGLEWVSRIGPSGGPTHYADSVKGRFTISRDN |
| DAR4_LC_N9 | | | SKNTLYLQMNSLRAEDTAVYYCAGYDSGYDYVAVAGP |
| 2Q_S72T_G7 | | | AEYFQHWGQGTLVTVSS |
| 7S | | | |
| HCDR1 | 10 | Combined | GFTFSHYMMA |
| HCDR2 | 6 | Combined | RIGPSGGPTHYADSVKG |
| HCDR3 | 4 | Combined | YDSGYDYVAVAGPAEYFQH |
| HCDR1 | 2 | Chothia | GFTFSHY |
| HCDR2 | 3 | Chothia | GPSGGP |
| HCDR3 | 4 | Chothia | YDSGYDYVAVAGPAEYFQH |
| HCDR1 | 2 | Kabat | GFTFSHY |
| HCDR2 | 3 | Kabat | GPSGGP |
| HCDR3 | 4 | Kabat | YDSGYDYVAVAGPAEYFQH |
| HCDR1 | 7 | IMGT | GFTFSHYM |
| HCDR2 | 8 | IMGT | IGPSGGPT |
| HCDR3 | 9 | IMGT | AGYDSGYDYVAVAGPAEYFQH |
| Anti- | 73 | VL | DIQMTQSPSSLSASVGDRVTITCRASQSISTWLAWYQ |
| EphA2_1C1 | | | QKPGKAPKLLIYKASNLHTGVPSRFSGSGSGTEFTLT |
| DAR4_LC_N9 | | | ISSLQPDDFATYYCQQYQSYSRTFGQGTKVEIK |
| 2Q_S72T_G7 | | | |
| 7S | | | |
| LCDR1 | 15 | Combined | RASQSISTWLA |
| LCDR2 | 16 | Combined | KASNLHT |
| LCDR3 | 36 | Combined | QQYQSYSRT |
| LCDR1 | 12 | Chothia | SQSISTW |
| LCDR2 | 13 | Chothia | KAS |
| LCDR3 | 35 | Chothia | YQSYSR |
| LCDR1 | 12 | Kabat | SQSISTW |
| LCDR2 | 13 | Kabat | KAS |
| LCDR3 | 35 | Kabat | YQSYSR |
| LCDR1 | 18 | IMGT | QSISTW |
| LCDR2 | 13 | IMGT | KAS |
| LCDR3 | 36 | IMGT | QQYQSYSRT |

TABLE D
amino acid and nucleic acid sequences of full length mAb IgG chains

| Ab | SEQ ID NO | IgG chain | Amino acid sequence |
|---|---|---|---|
| EphA2 | 37 | Heavy | EVQLLESGGGLVQPGGSLRLSCAASGFTFSHYMMAW |
| (E152C | | Chain | VRQAPGKGLEWVSRIGPSGGPTHYADSVKGRFTISR |
| S375C) 1C1 | | (Wild | DNSKNTLYLQMNSLRAEDTAVYYCAGYDSGYDYVAV |
| | | Type Fc) | AGPAEYFQHWGQGTLVTVSSASTKGPSVFPLAPSSK |
| | | | STSGGTAALGCLVKDYFPCPVTVSWNSGALTSGVHT |
| | | | FPAVLQSSGLYSLSSVVTVPSSSLGTQTYICNVNHK |
| | | | PSNTKVDKRVEPKSCDKTHTCPPCPAPELLGGPSVF |
| | | | LFPPKPKDTLMISRTPEVTCVVVDVSHEDPEVKENW |
| | | | YVDGVEVHNAKTKPREEQYNSTYRVVSVLTVLHQDW |
| | | | LNGKEYKCKVSNKALPAPIEKTISKAKGQPREPQVY |
| | | | TLPPSREEMTKNQVSLTCLVKGFYPCDIAVEWESNG |
| | | | QPENNYKTTPPVLDSDGSFFLYSKLTVDKSRWQQGN |
| | | | VFSCSVMHEALHNHYTQKSLSLSPGK |
| | 38 | DNA | GAAGTTCAGCTGCTTGAATCTGGCGGCGGACTGGTT |
| | | Heavy | CAACCTGGCGGATCTCTGAGACTGAGCTGTGCCGCC |
| | | Chain | AGCGGCTTCACCTTCAGCCACTATATGATGGCCTGG |
| | | | GTCCGACAGGCCCCTGGCAAAGGACTTGAATGGGTG |
| | | | TCCAGAATCGGCCCCTCTGGCGGCCCTACACACTAC |
| | | | GCTGATTCTGTGAAGGGCAGATTCACCATCAGCCGG |
| | | | GACAACAGCAAGAACACCCTGTACCTGCAGATGAAC |
| | | | AGCCTGAGAGCCGAGGACACCGCCGTGTATTACTGT |
| | | | GCCGGCTACGACAGCGGCTACGATTATGTGGCTGTG |
| | | | GCCGGACCTGCCGAGTACTTTCAGCATTGGGGACAG |
| | | | GGCACCCTGGTCACCGTTAGTTCTGCTAGCACCAAG |
| | | | GGCCCAAGTGTGTTTCCCCTGGCCCCCAGCAGCAAG |
| | | | TCTACTTCCGGCGGAACTGCTGCCCTGGGTTGCCTG |
| | | | GTGAAGGACTACTTCCCCTGTCCCGTGACAGTGTCC |
| | | | TGGAACTCTGGGGCTCTGACTTCCGGCGTGCACACC |
| | | | TTCCCCGCCGTGCTGCAGAGCAGCGGCCTGTACAGC |
| | | | CTGAGCAGCGTGGTGACAGTGCCCTCCAGCTCTCTG |
| | | | GGAACCCAGACCTATATCTGCAACGTGAACCACAAG |
| | | | CCCAGCAACACCAAGGTGGACAAGAGAGTGGAGCCC |
| | | | AAGAGCTGCGACAAGACCCACACCTGCCCCCCCTGC |
| | | | CCAGCTCCAGAACTGCTGGGAGGGCCTTCCGTGTTC |
| | | | CTGTTCCCCCCCAAGCCCAAGGACACCCTGATGATC |
| | | | AGCAGGACCCCCGAGGTGACCTGCGTGGTGGTGGAC |
| | | | GTGTCCCACGAGGACCCAGAGGTGAAGTTCAACTGG |
| | | | TACGTGGACGGCGTGGAGGTGCACAACGCCAAGACC |
| | | | AAGCCCAGAGAGGAGCAGTACAACAGCACCTACAGG |
| | | | GTGGTGTCCGTGCTGACCGTGCTGCACCAGGACTGG |
| | | | CTGAACGGCAAAGAATACAAGTGCAAAGTCTCCAAC |
| | | | AAGGCCCTGCCAGCCCCAATCGAAAAGACAATCAGC |
| | | | AAGGCCAAGGGCCAGCCACGGGAGCCCCAGGTGTAC |
| | | | ACCCTGCCCCCCAGCCGGGAGGAGATGACCAAGAAC |
| | | | CAGGTGTCCCTGACCTGTCTGGTGAAGGGCTTCTAC |
| | | | CCCTGTGATATCGCCGTGGAGTGGGAGAGCAACGGC |
| | | | CAGCCCGAGAACAACTACAAGACCACCCCCCCAGTG |
| | | | CTGGACAGCGACGGCAGCTTCTTCCTGTACAGCAAG |
| | | | CTGACCGTGGACAAGTCCAGGTGGCAGCAGGGCAAC |
| | | | GTGTTCAGCTGCAGCGTGATGCACGAGGCCCTGCAC |
| | | | AACCACTACACCCAGAAGTCCCTGAGCCTGAGCCCC |
| | | | GGCAAG |
| EphA2 | 39 | Heavy | EVQLLESGGGLVQPGGSLRLSCAASGFTFSHYMMAW |
| (E152C | | Chain | VRQAPGKGLEWVSRIGPSGGPTHYADSVKGRFTISR |
| S375C) | | | DNSKNTLYLQMNSLRAEDTAVYYCAGYDSGYDYVAV |
| DANAPA 1C1 | | | AGPAEYFQHWGQGTLVTVSSASTKGPSVFPLAPSSK |
| | | | STSGGTAALGCLVKDYFPCPVTVSWNSGALTSGVHT |
| | | | FPAVLQSSGLYSLSSVVTVPSSSLGTQTYICNVNHK |
| | | | PSNTKVDKRVEPKSCDKTHTCPPCPAPELLGGPSVF |
| | | | LFPPKPKDTLMISRTPEVTCVVVAVSHEDPEVKENW |
| | | | YVDGVEVHNAKTKPREEQYASTYRVVSVLTVLHQDW |
| | | | LNGKEYKCKVSNKALAAPIEKTISKAKGQPREPQVY |
| | | | TLPPSREEMTKNQVSLTCLVKGFYPCDIAVEWESNG |
| | | | QPENNYKTTPPVLDSDGSFFLYSKLTVDKSRWQQGN |
| | | | VFSCSVMHEALHNHYTQKSLSLSPGK |
| | 40 | DNA | GAAGTTCAGCTGCTTGAATCTGGCGGCGGACTGGTT |
| | | Heavy | CAACCTGGCGGATCTCTGAGACTGAGCTGTGCCGCC |
| | | Chain | AGCGGCTTCACCTTCAGCCACTATATGATGGCCTGG |
| | | | GTCCGACAGGCCCCTGGCAAAGGACTTGAATGGGTG |
| | | | TCCAGAATCGGCCCCTCTGGCGGCCCTACACACTAC |
| | | | GCTGATTCTGTGAAGGGCAGATTCACCATCAGCCGG |
| | | | GACAACAGCAAGAACACCCTGTACCTGCAGATGAAC |
| | | | AGCCTGAGAGCCGAGGACACCGCCGTGTATTACTGT |
| | | | GCCGGCTACGACAGCGGCTACGATTATGTGGCTGTG |
| | | | GCCGGACCTGCCGAGTACTTTCAGCATTGGGGACAG |
| | | | GGCACCCTGGTCACCGTTAGTTCTGCTAGCACCAAG |
| | | | GGCCCAAGTGTGTTTCCCCTGGCCCCCAGCAGCAAG |
| | | | TCTACTTCCGGCGGAACTGCTGCCCTGGGTTGCCTG |
| | | | GTGAAGGACTACTTCCCCTGTCCCGTGACAGTGTCC |
| | | | TGGAACTCTGGGGCTCTGACTTCCGGCGTGCACACC |
| | | | TTCCCCGCCGTGCTGCAGAGCAGCGGCCTGTACAGC |
| | | | CTGAGCAGCGTGGTGACAGTGCCCTCCAGCTCTCTG |
| | | | GGAACCCAGACCTATATCTGCAACGTGAACCACAAG |
| | | | CCCAGCAACACCAAGGTGGACAAGAGAGTGGAGCCC |
| | | | AAGAGCTGCGACAAGACCCACACCTGCCCCCCCTGC |
| | | | CCAGCTCCAGAACTGCTGGGAGGGCCTTCCGTGTTC |
| | | | CTGTTCCCCCCCAAGCCCAAGGACACCCTGATGATC |
| | | | AGCAGGACCCCCGAGGTGACCTGCGTGGTGGTGGCC |
| | | | GTGTCCCACGAGGACCCAGAGGTGAAGTTCAACTGG |
| | | | TACGTGGACGGCGTGGAGGTGCACAACGCCAAGACC |
| | | | AAGCCCAGAGAGGAGCAGTACGCCAGCACCTACAGG |
| | | | GTGGTGTCCGTGCTGACCGTGCTGCACCAGGACTGG |
| | | | CTGAACGGCAAAGAATACAAGTGCAAAGTCTCCAAC |
| | | | AAGGCCCTGGCTGCCCCAATCGAAAAGACAATCAGC |
| | | | AAGGCCAAGGGCCAGCCACGGGAGCCCCAGGTGTAC |
| | | | ACCCTGCCCCCCAGCCGGGAGGAGATGACCAAGAAC |
| | | | CAGGTGTCCCTGACCTGTCTGGTGAAGGGCTICTAC |
| | | | CCCTGTGATATCGCCGTGGAGTGGGAGAGCAACGGC |
| | | | CAGCCCGAGAACAACTACAAGACCACCCCCCCAGTG |
| | | | CTGGACAGCGACGGCAGCTTCTTCCTGTACAGCAAG |
| | | | CTGACCGTGGACAAGTCCAGGTGGCAGCAGGGCAAC |
| | | | GTGTTCAGCTGCAGCGTGATGCACGAGGCCCTGCAC |
| | | | AACCACTACACCCAGAAGTCCCTGAGCCTGAGCCCC |
| | | | GGCAAG |
| WT Light | 41 | Light | DIQMTQSPSSLSASVGDRVTITCRASQSISTWLAWY |
| Chain | | Chain | QQKPGKAPKLLIYKASNLHTGVPSRFSGSGSGTEFS |
| | | | LTISGLQPDDFATYYCQQYNSYSRTFGQGTKVEIKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSFNRGEC |
| | 42 | DNA | GACATTCAGATGACACAGAGCCCTAGCAGCCTGAGC |
| | | Light | GCCTCTGTGGGAGACAGAGTGACCATCACCTGTAGA |
| | | Chain | GCCAGCCAGAGCATCAGCACATGGCTGGCCTGGTAT |
| | | | CAGCAGAAGCCTGGCAAGGCCCCTAAGCTGCTGATC |
| | | | TACAAGGCCAGCAATCTGCACACCGGCGTGCCCAGC |
| | | | AGATTTTCTGGCTCTGGAAGCGGCACCGAGTTCAGC |
| | | | CTGACAATCTCTGGCCTGCAGCCTGACGACTTCGCC |
| | | | ACCTACTACTGCCAGCAGTACAACAGCTACAGCCGG |
| | | | ACCTTTGGCCAGGGAACAAAGGTGGAAATCAAGCGT |
| | | | ACGGTGGCCGCTCCCAGCGTGTTCATCTTCCCCCCC |
| | | | AGCGACGAGCAGCTGAAGAGTGGCACCGCCAGCGTG |
| | | | GTGTGCCTGCTGAACAACTTCTACCCCCGGGAGGCC |
| | | | AAGGTGCAGTGGAAGGTGGACAACGCCCTGCAGAGC |
| | | | GGCAACAGCCAGGAGAGCGTCACCGAGCAGGACAGC |
| | | | AAGGACTCCACCTACAGCCTGAGCAGCACCCTGACC |
| | | | CTGAGCAAGGCCGACTACGAGAAGCATAAGGTGTAC |
| | | | GCCTGCGAGGTGACCCACCAGGGCCTGTCCAGCCCC |
| | | | GTGACCAAGAGCTTCAACAGGGGCGAGTGC |
| Light | 43 | Light | DIQMTQSPSTLSASVGDRVTITCRASQSISTWLAWY |
| Chain S10T | | Chain | QQKPGKAPKLLIYKASNLHTGVPSRFSGSGSGTEFS |
| | | (S10T) | LTISGLQPDDFATYYCQQYNSYSRTFGQGTKVEIKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSENRGEC |
| | 44 | DNA | ACATTCAGATGACACAGAGCCCTAGCACACTGAGCG |
| | | Light | CCAGCGTGGGAGACAGAGTGACCATCACATGTAGAG |
| | | Chain | CCAGCCAGAGCATCAGCACCTGGCTGGCATGGTATC |
| | | | AGCAGAAGCCTGGCAAGGCCCCTAAGCTGCTGATCT |
| | | | ACAAGGCCAGCAATCTGCACACCGGCGTGCCCAGCA |
| | | | GATTTTCTGGCTCTGGAAGCGGCACCGAGTTCAGCC |
| | | | TGACAATCTCTGGCCTGCAGCCTGACGACTTCGCCA |
| | | | CCTACTACTGCCAGCAGTACAACAGCTACAGCCGGA |
| | | | CCTTTGGCCAGGGAACAAAGGTGGAAATCAAGCGTA |
| | | | CGTGGCCGCTCCCAGCGTGTTCATCTTCCCCCCCAG |
| | | | CGACGAGCAGCTGAAGAGTGGCACCGCCAGCGTGGT |
| | | | GTGCCTGCTGAACAACTTCTACCCCCGGGAGGCCAA |
| | | | GGTGCAGTGGAAGGTGGACAACGCCCTGCAGAGCGG |
| | | | CAACAGCCAGGAGAGCGTCACCGAGCAGGACAGCAA |
| | | | GGACTCCACCTACAGCCTGAGCAGCACCCTGACCCT |
| | | | GAGCAAGGCCGACTACGAGAAGCATAAGGTGTACGC |
| | | | CTGCGAGGTGACCCACCAGGGCCTGTCCAGCCCCGT |
| | | | GACCAAGAGCTTCAACAGGGGCGAGTGC |
| Light | 45 | Light | DIQMTQSPSSLSASVGDRVTITCRASQSISTWLAWY |
| Chain S72T | | Chain | QQKPGKAPKLLIYKASNLHTGVPSRFSGSGSGTEFT |
| | | (S72T) | LTISGLQPDDFATYYCQQYNSYSRTFGQGTKVEIKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSENRGEC |
| | 46 | DNA | GACATTCAGATGACACAGAGCCCTAGCAGCCTGAGC |
| | | Light | GCCTCTGTGGGAGACAGAGTGACCATCACCTGTAGA |
| | | Chain | GCCAGCCAGAGCATCAGCACATGGCTGGCCTGGTAT |
| | | | CAGCAGAAGCCTGGCAAGGCCCCTAAGCTGCTGATC |
| | | | TACAAGGCCAGCAATCTGCACACCGGCGTGCCCAGC |
| | | | AGATTTTCTGGCTCTGGAAGCGGCACCGAGTTCACC |
| | | | CTGACAATCTCTGGCCTGCAGCCTGACGACTTCGCC |
| | | | ACCTACTACTGCCAGCAGTACAACAGCTACAGCCGG |
| | | | ACCTTTGGCCAGGGAACAAAGGTGGAAATCAAGCGT |
| | | | ACGTGGCCGCTCCCAGCGTGTTCATCTTCCCCCCCA |
| | | | GCGACGAGCAGCTGAAGAGTGGCACCGCCAGCGTGG |
| | | | TGTGCCTGCTGAACAACTTCTACCCCCGGGAGGCCA |
| | | | AGGTGCAGTGGAAGGTGGACAACGCCCTGCAGAGCG |
| | | | GCAACAGCCAGGAGAGCGTCACCGAGCAGGACAGCA |
| | | | AGGACTCCACCTACAGCCTGAGCAGCACCCTGACCC |
| | | | TGAGCAAGGCCGACTACGAGAAGCATAAGGTGTACG |
| | | | CCTGCGAGGTGACCCACCAGGGCCTGTCCAGCCCCG |
| | | | TGACCAAGAGCTTCAACAGGGGCGAGTGC |
| Light | 47 | Light | DIQMTQSPSSLSASVGDRVTITCRASQSISTWLAWY |
| Chain G77S | | Chain | QQKPGKAPKLLIYKASNLHTGVPSRFSGSGSGTEFS |
| | | (G77S) | LTISSLQPDDFATYYCQQYNSYSRTFGQGTKVEIKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSENRGEC |
| | 48 | DNA | GACATTCAGATGACACAGAGCCCTAGCAGCCTGAGC |
| | | Light | GCCTCTGTGGGAGACAGAGTGACCATCACCTGTAGA |
| | | Chain | GCCAGCCAGAGCATCAGCACATGGCTGGCCTGGTAT |
| | | | CAGCAGAAGCCTGGCAAGGCCCCTAAGCTGCTGATC |
| | | | TACAAGGCCAGCAATCTGCACACCGGCGTGCCCAGC |
| | | | AGATTTTCTGGCTCTGGAAGCGGCACCGAGTTCAGC |
| | | | CTGACAATCAGCAGCCTGCAGCCTGACGACTTCGCC |
| | | | ACCTACTACTGCCAGCAGTACAACAGCTACAGCCGG |
| | | | ACCTTTGGCCAGGGAACAAAGGTGGAAATCAAGCGT |
| | | | ACGTGGCCGCTCCCAGCGTGTTCATCTTCCCCCCCA |
| | | | GCGACGAGCAGCTGAAGAGTGGCACCGCCAGCGTGG |
| | | | TGTGCCTGCTGAACAACTTCTACCCCCGGGAGGCCA |
| | | | AGGTGCAGTGGAAGGTGGACAACGCCCTGCAGAGCG |
| | | | GCAACAGCCAGGAGAGCGTCACCGAGCAGGACAGCA |
| | | | AGGACTCCACCTACAGCCTGAGCAGCACCCTGACCC |
| | | | TGAGCAAGGCCGACTACGAGAAGCATAAGGTGTACG |
| | | | CCTGCGAGGTGACCCACCAGGGCCTGTCCAGCCCCG |
| | | | TGACCAAGAGCTTCAACAGGGGCGAGTGC |
| Light | 49 | Light | DIQMTQSPSTLSASVGDRVTITCRASQSISTWLAWY |
| Chain | | Chain | QQKPGKAPKLLIYKASNLHTGVPSRFSGSGSGTEFT |
| S10T_S72T | | (LC | LTISSLQPDDFATYYCQQYNSYSRTFGQGTKVEIKR |
| G77S | | S10T_S72 | TVAAPSVFIFPPSDEQLKSGTASVVCLLNNFYPREA |
| | | T_G77S) | KVQWKVDNALQSGNSQESVTEQDSKDSTYSLSSTLT |
| | | | LSKADYEKHKVYACEVTHQGLSSPVTKSENRGEC |
| | 50 | DNA | GACATTCAGATGACACAGAGCCCTAGCACACTGAGC |
| | | Light | GCCAGCGTGGGAGACAGAGTGACCATCACATGTAGA |
| | | Chain | GCCAGCCAGAGCATCAGCACCTGGCTGGCATGGTAT |
| | | | CAGCAGAAGCCTGGCAAGGCCCCTAAGCTGCTGATC |
| | | | TACAAGGCCAGCAATCTGCACACCGGCGTGCCCAGC |
| | | | AGATTTTCTGGCTCTGGAAGCGGCACCGAGTTCACC |
| | | | CTGACCATCAGTAGCCTGCAGCCTGACGACTTCGCC |
| | | | ACCTACTACTGCCAGCAGTACAACAGCTACAGCCGG |
| | | | ACCTTTGGCCAGGGAACAAAGGTGGAAATCAAGCGT |
| | | | ACGTGGCCGCTCCCAGCGTGTTCATCTTCCCCCCCA |
| | | | GCGACGAGCAGCTGAAGAGTGGCACCGCCAGCGTGG |
| | | | TGTGCCTGCTGAACAACTTCTACCCCCGGGAGGCCA |
| | | | AGGTGCAGTGGAAGGTGGACAACGCCCTGCAGAGCG |
| | | | GCAACAGCCAGGAGAGCGTCACCGAGCAGGACAGCA |
| | | | AGGACTCCACCTACAGCCTGAGCAGCACCCTGACCC |
| | | | TGAGCAAGGCCGACTACGAGAAGCATAAGGTGTACG |
| | | | CCTGCGAGGTGACCCACCAGGGCCTGTCCAGCCCCG |
| | | | TGACCAAGAGCTTCAACAGGGGCGAGTGC |
| Light | 51 | Light | DIQMTQSPSSLSASVGDRVTITCRASQSISTWLAWY |
| Chain S93Q | | Chain | QQKPGKAPKLLIYKASNLHTGVPSRFSGSGSGTEFS |
| | | (S93Q) | LTISGLQPDDFATYYCQQYNQYSRTFGQGTKVEIKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSENRGEC |
| | 52 | DNA | GACATTCAGATGACACAGAGCCCTAGCAGCCTGAGC |
| | | Light | GCCTCTGTGGGAGACAGAGTGACCATCACCTGTAGA |
| | | Chain | GCCAGCCAGAGCATCAGCACATGGCTGGCCTGGTAT |
| | | | CAGCAGAAGCCTGGCAAGGCCCCTAAGCTGCTGATC |
| | | | TACAAGGCCAGCAATCTGCACACCGGCGTGCCCAGC |
| | | | AGATTTTCTGGCTCTGGAAGCGGCACCGAGTTCAGC |
| | | | CTGACAATCTCTGGCCTGCAGCCTGACGACTTCGCC |
| | | | ACCTACTACTGCCAGCAGTACAACCAGTACAGCCGG |
| | | | ACCTTTGGCCAGGGAACAAAGGTGGAAATCAAGCGT |
| | | | ACGTGGCCGCTCCCAGCGTGTTCATCTTCCCCCCCA |
| | | | GCGACGAGCAGCTGAAGAGTGGCACCGCCAGCGTGG |
| | | | TGTGCCTGCTGAACAACTTCTACCCCCGGGAGGCCA |
| | | | AGGTGCAGTGGAAGGTGGACAACGCCCTGCAGAGCG |
| | | | GCAACAGCCAGGAGAGCGTCACCGAGCAGGACAGCA |
| | | | AGGACTCCACCTACAGCCTGAGCAGCACCCTGACCC |
| | | | TGAGCAAGGCCGACTACGAGAAGCATAAGGTGTACG |
| | | | CCTGCGAGGTGACCCACCAGGGCCTGTCCAGCCCCG |
| | | | TGACCAAGAGCTTCAACAGGGGCGAGTGC |
| Light | 53 | Light | DIQMTQSPSSLSASVGDRVTITCRASQSISTWLAWY |
| Chain S93V | | Chain | QQKPGKAPKLLIYKASNLHTGVPSRFSGSGSGTEFS |
| | | (S93V) | LTISGLQPDDFATYYCQQYNVYSRTFGQGTKVEIKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSENRGEC |
| | 54 | DNA | GACATTCAGATGACACAGAGCCCTAGCAGCCTGAGC |
| | | Light | GCCTCTGTGGGAGACAGAGTGACCATCACCTGTAGA |
| | | Chain | GCCAGCCAGAGCATCAGCACATGGCTGGCCTGGTAT |
| | | | CAGCAGAAGCCTGGCAAGGCCCCTAAGCTGCTGATC |
| | | | TACAAGGCCAGCAATCTGCACACCGGCGTGCCCAGC |
| | | | AGATTTTCTGGCTCTGGAAGCGGCACCGAGTTCAGC |
| | | | CTGACAATCTCTGGCCTGCAGCCTGACGACTTCGCC |
| | | | ACCTACTACTGCCAGCAGTACAACGTGTACAGCCGG |
| | | | ACCTTTGGCCAGGGAACAAAGGTGGAAATCAAGCGT |
| | | | ACGTGGCCGCTCCCAGCGTGTTCATCTTCCCCCCCA |
| | | | GCGACGAGCAGCTGAAGAGTGGCACCGCCAGCGTGG |
| | | | TGTGCCTGCTGAACAACTTCTACCCCCGGGAGGCCA |
| | | | AGGTGCAGTGGAAGGTGGACAACGCCCTGCAGAGCG |
| | | | GCAACAGCCAGGAGAGCGTCACCGAGCAGGACAGCA |
| | | | AGGACTCCACCTACAGCCTGAGCAGCACCCTGACCC |
| | | | TGAGCAAGGCCGACTACGAGAAGCATAAGGTGTACG |
| | | | CCTGCGAGGTGACCCACCAGGGCCTGTCCAGCCCCG |
| | | | TGACCAAGAGCTTCAACAGGGGCGAGTGC |
| Light | 55 | Light | DIQMTQSPSSLSASVGDRVTITCRASQSISTWLAWY |
| Chain S93A | | Chain | QQKPGKAPKLLIYKASNLHTGVPSRFSGSGSGTEFS |
| | | (S93A) | LTISGLQPDDFATYYCQQYNAYSRTFGQGTKVEIKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSENRGEC |
| | 56 | DNA | GACATTCAGATGACACAGAGCCCTAGCAGCCTGAGC |
| | | Light | GCCTCTGTGGGAGACAGAGTGACCATCACCTGTAGA |
| | | Chain | GCCAGCCAGAGCATCAGCACATGGCTGGCCTGGTAT |
| | | | CAGCAGAAGCCTGGCAAGGCCCCTAAGCTGCTGATC |
| | | | TACAAGGCCAGCAATCTGCACACCGGCGTGCCCAGC |
| | | | AGATTTTCTGGCTCTGGAAGCGGCACCGAGTTCAGC |
| | | | CTGACAATCTCTGGCCTGCAGCCTGACGACTTCGCC |
| | | | ACCTACTACTGCCAGCAGTACAACGCCTACAGCCGG |
| | | | ACATTTGGCCAGGGAACAAAGGTGGAAATCAAGCGT |
| | | | ACGTGGCCGCTCCCAGCGTGTTCATCTTCCCCCCCA |
| | | | GCGACGAGCAGCTGAAGAGTGGCACCGCCAGCGTGG |
| | | | TGTGCCTGCTGAACAACTTCTACCCCCGGGAGGCCA |
| | | | AGGTGCAGTGGAAGGTGGACAACGCCCTGCAGAGCG |
| | | | GCAACAGCCAGGAGAGCGTCACCGAGCAGGACAGCA |
| | | | AGGACTCCACCTACAGCCTGAGCAGCACCCTGACCC |
| | | | TGAGCAAGGCCGACTACGAGAAGCATAAGGTGTACG |
| | | | CCTGCGAGGTGACCCACCAGGGCCTGTCCAGCCCCG |
| | | | TGACCAAGAGCTTCAACAGGGGCGAGTGC |
| Light | 57 | Light | DIQMTQSPSSLSASVGDRVTITCRASQSISTWLAWY |
| Chain N92A | | Chain | QQKPGKAPKLLIYKASNLHTGVPSRFSGSGSGTEFS |
| | | (N92A) | LTISGLQPDDFATYYCQQYASYSRTFGQGTKVEIKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSFNRGECRT |
| | | | VAAPSVFIFPPSDEQLKSGTASVVCLLNNFYPREAK |
| | | | VQWKVDNALQSGNSQESVTEQDSKDSTYSLSSTLTL |
| | | | SKADYEKHKVYACEVTHQGLSSPVTKSFNRGEC |
| | 58 | DNA | GACATTCAGATGACACAGAGCCCTAGCAGCCTGAGC |
| | | Light | GCCTCTGTGGGAGACAGAGTGACCATCACCTGTAGA |
| | | Chain | GCCAGCCAGAGCATCAGCACATGGCTGGCCTGGTAT |
| | | | CAGCAGAAGCCTGGCAAGGCCCCTAAGCTGCTGATC |
| | | | TACAAGGCCAGCAATCTGCACACCGGCGTGCCCAGC |
| | | | AGATTTTCTGGCTCTGGAAGCGGCACCGAGTTCAGC |
| | | | CTGACAATCTCTGGCCTGCAGCCTGACGACTTCGCC |
| | | | ACCTACTACTGTCAGCAGTACGCCAGCTACAGCCGG |
| | | | ACATTTGGCCAGGGAACAAAGGTGGAAATCAAGCGT |
| | | | ACGTGGCCGCTCCCAGCGTGTTCATCTTCCCCCCCA |
| | | | GCGACGAGCAGCTGAAGAGTGGCACCGCCAGCGTGG |
| | | | TGTGCCTGCTGAACAACTTCTACCCCCGGGAGGCCA |
| | | | AGGTGCAGTGGAAGGTGGACAACGCCCTGCAGAGCG |
| | | | GCAACAGCCAGGAGAGCGTCACCGAGCAGGACAGCA |
| | | | AGGACTCCACCTACAGCCTGAGCAGCACCCTGACCC |
| | | | TGAGCAAGGCCGACTACGAGAAGCATAAGGTGTACG |
| | | | CCTGCGAGGTGACCCACCAGGGCCTGTCCAGCCCCG |
| | | | TGACCAAGAGCTTCAACAGGGGCGAGTGC |
| Light | 59 | Light | DIQMTQSPSSLSASVGDRVTITCRASQSISTWLAWY |
| Chain N92Q | | Chain | QQKPGKAPKLLIYKASNLHTGVPSRFSGSGSGTEFS |
| | | (N92Q) | LTISGLQPDDFATYYCQQYQSYSRTFGQGTKVEIKR |
| | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSENRGEC |
| | 60 | DNA | GACATTCAGATGACACAGAGCCCTAGCAGCCTGAGC |
| | | Light | GCCTCTGTGGGAGACAGAGTGACCATCACCTGTAGA |
| | | Chain | GCCAGCCAGAGCATCAGCACATGGCTGGCCTGGTAT |
| | | | CAGCAGAAGCCTGGCAAGGCCCCTAAGCTGCTGATC |
| | | | TACAAGGCCAGCAATCTGCACACCGGCGTGCCCAGC |
| | | | AGATTTTCTGGCTCTGGAAGCGGCACCGAGTTCAGC |
| | | | CTGACAATCTCTGGCCTGCAGCCTGACGACTTCGCC |
| | | | ACCTACTACTGCCAGCAGTACCAGAGCTACAGCCGG |
| | | | ACATTTGGCCAGGGAACAAAGGTGGAAATCAAGCGT |
| | | | ACGTGGCCGCTCCCAGCGTGTTCATCTTCCCCCCCA |
| | | | GCGACGAGCAGCTGAAGAGTGGCACCGCCAGCGTGG |
| | | | TGTGCCTGCTGAACAACTTCTACCCCCGGGAGGCCA |
| | | | AGGTGCAGTGGAAGGTGGACAACGCCCTGCAGAGCG |
| | | | GCAACAGCCAGGAGAGCGTCACCGAGCAGGACAGCA |
| | | | AGGACTCCACCTACAGCCTGAGCAGCACCCTGACCC |
| | | | TGAGCAAGGCCGACTACGAGAAGCATAAGGTGTACG |
| | | | CCTGCGAGGTGACCCACCAGGGCCTGTCCAGCCCCG |
| | | | TGACCAAGAGCTTCAACAGGGGCGAGTGC |
| anti-EphA2 | 74 | Heavy | EVQLLESGGGLVQPGGSLRLSCAASGFTFSHYMMAW |
| 1C1 hIgG1 | | Chain | VRQAPGKGLEWVSRIGPSGGPTHYADSVKGRFTISR |
| | | | DNSKNTLYLQMNSLRAEDTAVYYCAGYDSGYDYVAV |
| | | | AGPAEYFQHWGQGTLVTVSSASTKGPSVFPLAPSSK |
| | | | STSGGTAALGCLVKDYFPEPVTVSWNSGALTSGVHT |
| | | | FPAVLQSSGLYSLSSVVTVPSSSLGTQTYICNVNHK |
| | | | PSNTKVDKRVEPKSCDKTHTCPPCPAPELLGGPSVF |
| | | | LFPPKPKDTLMISRTPEVTCVVVDVSHEDPEVKENW |
| | | | YVDGVEVHNAKTKPREEQYNSTYRVVSVLTVLHQDW |
| | | | LNGKEYKCKVSNKALPAPIEKTISKAKGQPREPQVY |
| | | | TLPPSREEMTKNQVSLTCLVKGFYPSDIAVEWESNG |
| | | | QPENNYKTTPPVLDSDGSFFLYSKLTVDKSRWQQGN |
| | | | VFSCSVMHEALHNHYTQKSLSLSPGK |
| Anti- | 75 | Light | DIQMTQSPSSLSASVGDRVTITCRASQSISTWLAWY |
| EphA2_1C1 | | Chain | QQKPGKAPKLLIYKASNLHTGVPSRFSGSGSGTEFT |
| DAR4_LC_N9 | | | LTISGLQPDDFATYYCQQYQSYSRTFGQGTKVEIKR |
| 2Q_S72T | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSENRGEC |
| Anti- | 76 | Light | DIQMTQSPSSLSASVGDRVTITCRASQSISTWLAWY |
| EphA2_1C1_ | | Chain | QQKPGKAPKLLIYKASNLHTGVPSRFSGSGSGTEFS |
| DAR4_LC_N9 | | | LTISSLQPDDFATYYCQQYQSYSRTFGQGTKVEIKR |
| 2Q_G77S | | | TVAAPSVFIFPPSDEQLKSGTASVVCLLNNFYPREA |
| | | | KVQWKVDNALQSGNSQESVTEQDSKDSTYSLSSTLT |
| | | | LSKADYEKHKVYACEVTHQGLSSPVTKSENRGEC |
| Anti- | 77 | Light | DIQMTQSPSSLSASVGDRVTITCRASQSISTWLAWY |
| EphA2_1C1 | | Chain | QQKPGKAPKLLIYKASNLHTGVPSRFSGSGSGTEFT |
| DAR4_LC_N9 | | | LTISSLQPDDFATYYCQQYQSYSRTFGQGTKVEIKR |
| 2Q_S72T_G7 | | | TVAAPSVFIFPPSDEQLKSGTASVVCLLNNFYPREA |
| 7S | | | KVQWKVDNALQSGNSQESVTEQDSKDSTYSLSSTLT |
| | | | LSKADYEKHKVYACEVTHQGLSSPVTKSENRGEC |

In some embodiments, the anti-EphA2 antibody or antigen-binding fragment of an ADC disclosed herein may comprise any set of heavy and light chain variable domains listed in the tables above or a set of six CDRs from any set of heavy and light chain variable domains listed in the tables above. In some embodiments, the anti-EphA2 antibody or antigen-binding fragment of an ADC disclosed herein may comprise amino acid sequences that are conservatively modified and/or homologous to the sequences listed in the tables above, so long as the ADC retains the ability to bind to its target cancer antigen (e.g., with a K_{D} of less than 1×10⁻⁸ M) and retains one or more functional properties of the ADCs disclosed herein (e.g., ability to internalize, bind to an antigen target, e.g., an antigen expressed on a tumor or other cancer cell, etc.).

In some embodiments, the anti-EphA2 antibody or antigen-binding fragment of an ADC disclosed herein further comprises human heavy and light chain constant domains or fragments thereof. For instance, the anti-EphA2 antibody or antigen-binding fragment of the described ADCs may comprise a human IgG heavy chain constant domain (such as an IgG1) and a human kappa or lambda light chain constant domain. In some embodiments, the anti-EphA2 antibody or antigen-binding fragment of the described ADCs comprises a human immunoglobulin G subtype 1 (IgG1) heavy chain constant domain with a human Ig kappa light chain constant domain.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 2, heavy chain CDR2 (HCDR2) consisting of SEQ ID NO: 3, heavy chain CDR3 (HCDR3) consisting of SEQ ID NO: 4; light chain CDR1 (LCDR1) consisting of SEQ ID NO: 12, light chain CDR2 (LCDR2) consisting of SEQ ID NO: 13, and light chain CDR3 (LCDR3) consisting of SEQ ID NO: 14, SEQ ID NO: 26, SEQ ID NO: 29, SEQ ID NO: 32, SEQ ID NO: 35, or SEQ ID NO: 70.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 2, heavy chain CDR2 (HCDR2) consisting of SEQ ID NO: 3, heavy chain CDR3 (HCDR3) consisting of SEQ ID NO: 4; light chain CDR1 (LCDR1) consisting of SEQ ID NO: 12, light chain CDR2 (LCDR2) consisting of SEQ ID NO: 13, and light chain CDR3 (LCDR3) consisting of SEQ ID NO: 14.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 2, heavy chain CDR2 (HCDR2) consisting of SEQ ID NO: 3, heavy chain CDR3 (HCDR3) consisting of SEQ ID NO: 4; light chain CDR1 (LCDR1) consisting of SEQ ID NO: 12, light chain CDR2 (LCDR2) consisting of SEQ ID NO: 13, and light chain CDR3 (LCDR3) consisting of SEQ ID NO: 26.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 2, heavy chain CDR2 (HCDR2) consisting of SEQ ID NO: 3, heavy chain CDR3 (HCDR3) consisting of SEQ ID NO: 4; light chain CDR1 (LCDR1) consisting of SEQ ID NO: 12, light chain CDR2 (LCDR2) consisting of SEQ ID NO: 13, and light chain CDR3 (LCDR3) consisting of SEQ ID NO: 29.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 2, heavy chain CDR2 (HCDR2) consisting of SEQ ID NO: 3, heavy chain CDR3 (HCDR3) consisting of SEQ ID NO: 4; light chain CDR1 (LCDR1) consisting of SEQ ID NO: 12, light chain CDR2 (LCDR2) consisting of SEQ ID NO: 13, and light chain CDR3 (LCDR3) consisting of SEQ ID NO: 32.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 2, heavy chain CDR2 (HCDR2) consisting of SEQ ID NO: 3, heavy chain CDR3 (HCDR3) consisting of SEQ ID NO: 4; light chain CDR1 (LCDR1) consisting of SEQ ID NO: 12, light chain CDR2 (LCDR2) consisting of SEQ ID NO: 13, and light chain CDR3 (LCDR3) consisting of SEQ ID NO: 35.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 2, heavy chain CDR2 (HCDR2) consisting of SEQ ID NO: 3, heavy chain CDR3 (HCDR3) consisting of SEQ ID NO: 4; light chain CDR1 (LCDR1) consisting of SEQ ID NO: 12, light chain CDR2 (LCDR2) consisting of SEQ ID NO: 13, and light chain CDR3 (LCDR3) consisting of SEQ ID NO: 70.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 5, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 17, SEQ ID NO: 24, SEQ ID NO: 27, SEQ ID NO: 30, SEQ ID NO: 33, or SEQ ID NO: 36.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 5, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 17.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 5, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 24.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 5, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 27.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 5, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 30.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 5, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 33.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 5, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 36.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 7, heavy chain CDR2 (HCDR2) consisting of SEQ ID NO: 8, heavy chain CDR3 (HCDR3) consisting of SEQ ID NO: 9; light chain CDR1 (LCDR1) consisting of SEQ ID NO: 18, light chain CDR2 (LCDR2) consisting of SEQ ID NO: 13, and light chain CDR3 (LCDR3) consisting of SEQ ID NO: 17, SEQ ID NO: 24, SEQ ID NO: 27, SEQ ID NO: 30, SEQ ID NO: 33, or SEQ ID NO: 36.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 7, heavy chain CDR2 (HCDR2) consisting of SEQ ID NO: 8, heavy chain CDR3 (HCDR3) consisting of SEQ ID NO: 9; light chain CDR1 (LCDR1) consisting of SEQ ID NO: 18, light chain CDR2 (LCDR2) consisting of SEQ ID NO: 13, and light chain CDR3 (LCDR3) consisting of SEQ ID NO: 17.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 7, heavy chain CDR2 (HCDR2) consisting of SEQ ID NO: 8, heavy chain CDR3 (HCDR3) consisting of SEQ ID NO: 9; light chain CDR1 (LCDR1) consisting of SEQ ID NO: 18, light chain CDR2 (LCDR2) consisting of SEQ ID NO: 13, and light chain CDR3 (LCDR3) consisting of SEQ ID NO: 24.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 7, heavy chain CDR2 (HCDR2) consisting of SEQ ID NO: 8, heavy chain CDR3 (HCDR3) consisting of SEQ ID NO: 9; light chain CDR1 (LCDR1) consisting of SEQ ID NO: 18, light chain CDR2 (LCDR2) consisting of SEQ ID NO: 13, and light chain CDR3 (LCDR3) consisting of SEQ ID NO: 27.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 7, heavy chain CDR2 (HCDR2) consisting of SEQ ID NO: 8, heavy chain CDR3 (HCDR3) consisting of SEQ ID NO: 9; light chain CDR1 (LCDR1) consisting of SEQ ID NO: 18, light chain CDR2 (LCDR2) consisting of SEQ ID NO: 13, and light chain CDR3 (LCDR3) consisting of SEQ ID NO: 30.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 7, heavy chain CDR2 (HCDR2) consisting of SEQ ID NO: 8, heavy chain CDR3 (HCDR3) consisting of SEQ ID NO: 9; light chain CDR1 (LCDR1) consisting of SEQ ID NO: 18, light chain CDR2 (LCDR2) consisting of SEQ ID NO: 13, and light chain CDR3 (LCDR3) consisting of SEQ ID NO: 33.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 7, heavy chain CDR2 (HCDR2) consisting of SEQ ID NO: 8, heavy chain CDR3 (HCDR3) consisting of SEQ ID NO: 9; light chain CDR1 (LCDR1) consisting of SEQ ID NO: 18, light chain CDR2 (LCDR2) consisting of SEQ ID NO: 13, and light chain CDR3 (LCDR3) consisting of SEQ ID NO: 36.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 10, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 17, SEQ ID NO: 24, SEQ ID NO: 27, SEQ ID NO: 30, SEQ ID NO: 33, or SEQ ID NO: 36.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 10, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 17.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 10, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 24.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 10, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 27.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 10, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 30.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 10, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 33.

In some embodiments, the anti-EphA2 antibody or antigen-binding fragment thereof comprises three heavy chain CDRs and three light chain CDRs as follows: heavy chain CDR1 (HCDR1) consisting of SEQ ID NO: 10, heavy chain CDR2 (HCDR2) consisting of SEQ ID NO: 6, heavy chain CDR3 (HCDR3) consisting of SEQ ID NO: 4; light chain CDR1 (LCDR1) consisting of SEQ ID NO: 15, light chain CDR2 (LCDR2) consisting of SEQ ID NO: 16, and light chain CDR3 (LCDR3) consisting of SEQ ID NO: 36.

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 11. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 11, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 11

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 19. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 19, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 19.

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 20. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 20, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 20.

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 21. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 21, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 21.

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 22. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 22, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 22.

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 23. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 23, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 23.

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 25. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 25, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 25.

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 28. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 28, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 28.

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 31. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 31, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 31.

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 34. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 34, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 34.

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 71. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 71, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 71.

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 72. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 72, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 72.

In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 73. In some embodiments, the anti-EphA2 antibody or antigen-binding fragment thereof comprises the heavy chain variable region amino acid sequence of SEQ ID NO: 1 and the light chain variable region amino acid sequence of SEQ ID NO: 73, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 1 and/or a light chain variable region amino acid sequence that is at least 96%, at least 97%, at least 98%, or at least 99% identical to SEQ ID NO: 73.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77 or a sequence that is at least 95% identical to SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77. In some embodiments, the anti-EphA2 antibody comprises the heavy chain amino acid sequence of SEQ ID NO: 37 and the light chain amino acid sequence of SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77 or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 41 or a sequence that is at least 95% identical to SEQ ID NO: 41. In some embodiments, the anti-EphA2 antibody comprises the heavy chain amino acid sequence of SEQ ID NO: 37 and the light chain amino acid sequence of SEQ ID NO: 41, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 41.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 43 or a sequence that is at least 95% identical to SEQ ID NO: 43. In some embodiments, the anti-EphA2 antibody comprises the heavy chain amino acid sequence of SEQ ID NO: 37 and the light chain amino acid sequence of SEQ ID NO: 43, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 43.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 45 or a sequence that is at least 95% identical to SEQ ID NO: 45. In some embodiments, the anti-EphA2 antibody comprises the heavy chain amino acid sequence of SEQ ID NO: 37 and the light chain amino acid sequence of SEQ ID NO: 45, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 45.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 47 or a sequence that is at least 95% identical to SEQ ID NO: 47. In some embodiments, the anti-EphA2 antibody comprises the heavy chain amino acid sequence of SEQ ID NO: 37 and the light chain amino acid sequence of SEQ ID NO: 47, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 47.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 49 or a sequence that is at least 95% identical to SEQ ID NO: 49. In some embodiments, the anti-EphA2 antibody comprises the heavy chain amino acid sequence of SEQ ID NO: 37 and the light chain amino acid sequence of SEQ ID NO: 49, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 49.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 51 or a sequence that is at least 95% identical to SEQ ID NO: 51. In some embodiments, the anti-EphA2 antibody comprises the heavy chain amino acid sequence of SEQ ID NO: 37 and the light chain amino acid sequence of SEQ ID NO: 51, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 51.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 53 or a sequence that is at least 95% identical to SEQ ID NO: 53. In some embodiments, the anti-EphA2 antibody comprises the heavy chain amino acid sequence of SEQ ID NO: 37 and the light chain amino acid sequence of SEQ ID NO: 53, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 53.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 55 or a sequence that is at least 95% identical to SEQ ID NO: 55. In some embodiments, the anti-EphA2 antibody comprises the heavy chain amino acid sequence of SEQ ID NO: 37 and the light chain amino acid sequence of SEQ ID NO: 55, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 55.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 57 or a sequence that is at least 95% identical to SEQ ID NO: 57. In some embodiments, the anti-EphA2 antibody comprises the heavy chain amino acid sequence of SEQ ID NO: 37 and the light chain amino acid sequence of SEQ ID NO: 57, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 57.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 59 or a sequence that is at least 95% identical to SEQ ID NO: 59. In some embodiments, the anti-EphA2 antibody comprises the heavy chain amino acid sequence of SEQ ID NO: 37 and the light chain amino acid sequence of SEQ ID NO: 59, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 59.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 75 or a sequence that is at least 95% identical to SEQ ID NO: 75. In some embodiments, the anti-EphA2 antibody comprises the heavy chain amino acid sequence of SEQ ID NO: 37 and the light chain amino acid sequence of SEQ ID NO: 75, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 75.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 76 or a sequence that is at least 95% identical to SEQ ID NO: 76. In some embodiments, the anti-EphA2 antibody comprises the heavy chain amino acid sequence of SEQ ID NO: 37 and the light chain amino acid sequence of SEQ ID NO: 76, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 76.

In some embodiments, the anti-EphA2 antibody comprises the heavy chain amino acid sequence of SEQ ID NO: 37 or a sequence that is at least 95% identical to SEQ ID NO: 37, and the light chain amino acid sequence of SEQ ID NO: 77 or a sequence that is at least 95% identical to SEQ ID NO: 77. In some embodiments, the anti-EphA2 antibody comprises the heavy chain amino acid sequence of SEQ ID NO: 37 and the light chain amino acid sequence of SEQ ID NO: 77, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 37 and a light chain amino acid sequence that is at least 96%, at least 97%, at least 98%, or at least 99% identical to SEQ ID NO: 77.

In some embodiments, the anti-EphA2 antibody comprises the heavy chain amino acid sequence of SEQ ID NO: 39 or a sequence that is at least 95% identical to SEQ ID NO: 39, and the light chain amino acid sequence of SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77 or a sequence that is at least 95% identical to SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77. In some embodiments, the anti-EphA2 antibody comprises the heavy chain amino acid sequence of SEQ ID NO: 39 and the light chain amino acid sequence of SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77 or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 39 and a light chain amino acid sequence that is at least 96%, at least 97%, at least 98%, or at least 99% identical to SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77.

In some embodiments, the anti-EphA2 antibody comprises the heavy chain amino acid sequence of SEQ ID NO: 39 or a sequence that is at least 95% identical to SEQ ID NO: 39, and the light chain amino acid sequence of SEQ ID NO: 41 or a sequence that is at least 95% identical to SEQ ID NO: 41. In some embodiments, the anti-EphA2 antibody comprises the heavy chain amino acid sequence of SEQ ID NO: 39 and the light chain amino acid sequence of SEQ ID NO: 41, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 39 and a light chain amino acid sequence that is at least 96%, at least 97%, at least 98%, or at least 99% identical to SEQ ID NO: 41.

In some embodiments, the anti-EphA2 antibody comprises the heavy chain amino acid sequence of SEQ ID NO: 74 or a sequence that is at least 95% identical to SEQ ID NO: 74, and the light chain amino acid sequence of SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77 or a sequence that is at least 95% identical to SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77. In some embodiments, the anti-EphA2 antibody comprises the heavy chain amino acid sequence of SEQ ID NO: 74 and the light chain amino acid sequence of SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77 or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 74 and a light chain amino acid sequence that is at least 96%, at least 97%, at least 98%, or at least 99% identical to SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77.

In some embodiments, the anti-EphA2 antibody comprises the heavy chain amino acid sequence of SEQ ID NO: 74 or a sequence that is at least 95% identical to SEQ ID NO: 74, and the light chain amino acid sequence of SEQ ID NO: 41 or a sequence that is at least 95% identical to SEQ ID NO: 41. In some embodiments, the anti-EphA2 antibody comprises the heavy chain amino acid sequence of SEQ ID NO: 74 and the light chain amino acid sequence of SEQ ID NO: 41, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO: 74 and a light chain amino acid sequence that is at least 96%, at least 97%, at least 98%, or at least 99% identical to SEQ ID NO: 41.

Residues in two or more polypeptides are said to “correspond” if the residues occupy an analogous position in the polypeptide structures. Analogous positions in two or more polypeptides can be determined by aligning the polypeptide sequences based on amino acid sequence or structural similarities. Those skilled in the art understand that it may be necessary to introduce gaps in either sequence to produce a satisfactory alignment.

In some embodiments, amino acid substitutions are of single residues. Insertions usually will be on the order of from about 1 to about 20 amino acid residues, although considerably larger insertions may be tolerated as long as biological function is retained (e.g., binding to a target antigen). Deletions usually range from about 1 to about 20 amino acid residues, although in some cases deletions may be much larger. Substitutions, deletions, insertions, or any combination thereof may be used to arrive at a final derivative or variant. Generally, these changes are done on a few amino acids to minimize the alteration of the molecule, particularly the immunogenicity and specificity of the antigen binding protein. However, larger changes may be tolerated in certain circumstances. Conservative substitutions can be made in accordance with the following chart depicted as Table 2.

TABLE 2

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

In some embodiments where variant antibody sequences are used in an ADC, the variants typically exhibit the same qualitative biological activity and will elicit the same immune response, although variants may also be selected to modify the characteristics of the antigen binding proteins as needed. Alternatively, the variant may be designed such that the biological activity of the antigen binding protein is altered. For example, glycosylation sites may be altered or removed.

Various antibodies may be used with the ADCs used herein to target cancer cells. As shown below, the linker-payloads in the ADCs disclosed herein are surprisingly effective with different tumor antigen-targeting antibodies. Suitable antigens expressed on cancer cells but not healthy cells, or expressed on cancer cells at a higher level than on healthy cells, are known in the art, as are antibodies directed against them. Further antibodies against those antigen targets may be prepared by those of skill in the art. These antibodies may be used with the linkers and Bcl-xL inhibitor payloads disclosed herein. In some embodiments, the antibody or antigen-binding fragment targets EphA2 provided particularly improved drug: antibody ratio, aggregation level, stability (i.e., in vitro and in vivo stability), tumor targeting (i.e., cytotoxicity, potency), minimized off-target killing, and/or treatment efficacy. Improved treatment efficacy can be measured in vitro or in vivo, and may include reduced tumor growth rate and/or reduced tumor volume.

In some embodiments, alternate antibodies to the same targets or antibodies to different antigen targets are used and provide at least some of the favorable functional properties described above (e.g., improved stability, improved tumor targeting, improved treatment efficacy, etc.).

### Linkers

In some embodiments, the linker in an ADC is stable extracellularly in a sufficient manner to be therapeutically effective. In some embodiments, the linker is stable outside a cell, such that the ADC remains intact when present in extracellular conditions (e.g., prior to transport or delivery into a cell). The term “intact,” used in the context of an ADC, means that the antibody or antigen-binding fragment remains attached to the drug moiety (e.g., the Bcl-xL inhibitor).

As used herein, “stable,” in the context of a linker or ADC comprising a linker, means that no more than 20%, no more than about 15%, no more than about 10%, no more than about 5%, no more than about 3%, or no more than about 1% of the linkers (or any percentage in between) in a sample of ADC are cleaved (or in the case of an overall ADC are otherwise not intact) when the ADC is present in extracellular conditions. In some embodiments, the linkers and/or ADCs disclosed herein are stable compared to alternate linkers and/or ADCs with alternate linkers and/or Bcl-xL inhibitor payloads. In some embodiments, the ADCs disclosed herein can remain intact for more than about 48 hours, more than 60 hours, more than about 72 hours, more than about 84 hours, or more than about 96 hours.

Whether a linker is stable extracellularly can be determined, for example, by including an ADC in plasma for a predetermined time period (e.g., 2, 4, 6, 8, 16, 24, 48, or 72 hours) and then quantifying the amount of free drug moiety present in the plasma. Stability may allow the ADC time to localize to target cancer cells and prevent the premature release of the drug moiety, which could lower the therapeutic index of the ADC by indiscriminately damaging both normal and cancer tissues. In some embodiments, the linker is stable outside of a target cell and releases the drug moiety from the ADC once inside of the cell, such that the drug can bind to its target. Thus, an effective linker will: (i) maintain the specific binding properties of the antibody or antigen-binding fragment; (ii) allow delivery, e.g., intracellular delivery, of the drug moiety via stable attachment to the antibody or antigen-binding fragment; (iii) remain stable and intact until the ADC has been transported or delivered to its target site; and (iv) allow for the therapeutic effect, e.g., cytotoxic effect, of the drug moiety after cleavage or alternate release mechanism.

Linkers may impact the physico-chemical properties of an ADC. As many cytotoxic agents are hydrophobic in nature, linking them to the antibody with an additional hydrophobic moiety may lead to aggregation. ADC aggregates are insoluble and often limit achievable drug loading onto the antibody, which can negatively affect the potency of the ADC. Protein aggregates of biologics, in general, have also been linked to increased immunogenicity. As shown below, linkers disclosed herein result in ADCs with low aggregation levels and desirable levels of drug loading.

A linker may be “cleavable” or “non-cleavable” (Ducry and Stump (2010) Bioconjugate Chem. 21:5-13). Cleavable linkers are designed to release the drug moiety (e.g., a Bcl-xL inhibitor) when subjected to certain environment factors, e.g., when internalized into the target cell, whereas non-cleavable linkers generally rely on the degradation of the antibody or antigen-binding fragment itself.

The term “alkyl”, as used herein, refers to a straight or branched hydrocarbon chain radical consisting solely of carbon and hydrogen atoms, containing no unsaturation. The term “C₁-C₆alkyl”, as used herein, refers to a straight or branched hydrocarbon chain radical consisting solely of carbon and hydrogen atoms, containing no unsaturation, having from one to six carbon atoms, and which is attached to the rest of the molecule by a single bond. Non-limiting examples of “C₁-C₆alkyl” groups include methyl(a C₁alkyl), ethyl (a C₂alkyl), 1-methylethyl (a C₃alkyl), n-propyl (a C₃alkyl), isopropyl (a C₃alkyl), n-butyl(a C₄alkyl), isobutyl(a C₄alkyl), sec-butyl(a C₄alkyl), tert-butyl(a C₄alkyl), n-pentyl (a C₅alkyl), isopentyl (a C₅alkyl), neopentyl (a C₅alkyl) and hexyl (a C₆alkyl).

The term “alkenyl”, as used herein, refers to a straight or branched hydrocarbon chain radical group consisting solely of carbon and hydrogen atoms, containing at least one double bond. The term “C₂-C₆alkenyl”, as used herein, refers to a straight or branched hydrocarbon chain radical group consisting solely of carbon and hydrogen atoms, containing at least one double bond, having from two to six carbon atoms, which is attached to the rest of the molecule by a single bond. Non-limiting examples of “C₂-C₆alkenyl” groups include ethenyl (a C₂alkenyl), prop-1-enyl (a C₃alkenyl), but-1-enyl (a C₄alkenyl), pent-1-enyl (a C₅alkenyl), pent-4-enyl (a C₅alkenyl), penta-1,4-dienyl (a C₅alkenyl), hexa-1-enyl (a C₆alkenyl), hexa-2-enyl (a C₆alkenyl), hexa-3-enyl (a C₆alkenyl), hexa-1-,4-dienyl (a C₆alkenyl), hexa-1-,5-dienyl (a C₆alkenyl) and hexa-2-,4-dienyl (a C₆alkenyl). The term “C₂-C₆alkenyl”, as used herein, refers to a straight or branched hydrocarbon chain radical group consisting solely of carbon and hydrogen atoms, containing at least one double bond, having from two to three carbon atoms, which is attached to the rest of the molecule by a single bond. Non-limiting examples of “C₂-C₆alkenyl” groups include ethenyl (a C₂alkenyl) and prop-1-enyl (a C₃alkenyl).

The term “alkylene”, as used herein, refers to a bivalent straight or branched hydrocarbon chain radical consisting solely of carbon and hydrogen atoms and containing no unsaturation. The term “C₁-C₆alkylene”, as used herein, refers to a bivalent straight or branched hydrocarbon chain radical consisting solely of carbon and hydrogen atoms, containing no unsaturation, having from one to six carbon atoms. Non-limiting examples of “C₁-C₆alkylene” groups include methylene (a C₁alkylene), ethylene (a C₂alkylene), 1-methylethylene (a C₃alkylene), n-propylene (a C₃alkylene), isopropylene (a C₃alkylene), n-butylene (a C₄alkylene), isobutylene (a C₄alkylene), sec-butylene (a C₄alkylene), tert-butylene (a C₄alkylene), n-pentylene (a C₅alkylene), isopentylene (a C₅alkylene), neopentylene (a C₅alkylene), and hexylene (a C₆alkylene).

The term “alkenylene”, as used herein, refers to a bivalent straight or branched hydrocarbon chain radical consisting solely of carbon and hydrogen atoms and containing at least one double bond. The term “C₂-C₆alkenylene”, as used herein, refers to a bivalent straight or branched hydrocarbon chain radical group consisting solely of carbon and hydrogen atoms, containing at least one double bond, and having from two to six carbon atoms. Non-limiting examples of “C₂-C₆alkenylene” groups include ethenylene (a C₂alkenylene), prop-1-enylene (a C₃alkenylene), but-1-enylene (a C₄alkenylene), pent-1-enylene (a C₅alkenylene), pent-4-enylene (a C₅alkenylene), penta-1,4-dienylene (a C₅alkenylene), hexa-1-enylene (a C₆alkenylene), hexa-2-enylene (a C₆alkenylene), hexa-3-enylene (a C₆alkenylene), hexa-1-,4-dienylene (a C₆alkenylene), hexa-1-,5-dienylene (a C₆alkenylene) and hexa-2-,4-dienylene (a C₆alkenylene). The term “C₂-C₆alkenylene”, as used herein, refers to a bivalent straight or branched hydrocarbon chain radical group consisting solely of carbon and hydrogen atoms, containing at least one double bond, and having from two to three carbon atoms. Non-limiting examples of “C₂-C₆alkenylene” groups include ethenylene (a C₂alkenylene) and prop-1-enylene (a C₃alkenylene).

The term “cycloalkyl,” or “C₃-C₈cycloalkyl,” as used herein, refers to a saturated, monocyclic, fused bicyclic, fused tricyclic or bridged polycyclic ring system. Non-limiting examples of fused bicyclic or bridged polycyclic ring systems include bicyclo[1.1.1]pentane, bicyclo[2.1.1]hexane, bicyclo[2.2.1]heptane, bicyclo[3.1.1]heptane, bicyclo[3.2.1]octane, bicyclo[2.2.2]octane and adamantanyl. Non-limiting examples monocyclic C₃-C₈cycloalkyl groups include cyclopropyl, cyclobutyl, cyclopentyl, cyclohexyl, cycloheptyl and cyclooctyl groups.

The term “aryl” as used herein, refers to a phenyl, naphthyl, biphenyl or indenyl group.

The term “heteroaryl” as used herein, refers any mono- or bi-cyclic group composed of from 5 to 10 ring members, having at least one aromatic moiety and containing from 1 to 4 hetero atoms selected from oxygen, sulphur and nitrogen (including quaternary nitrogens).

The term “cycloalkyl” as used herein, refers to any mono- or bi-cyclic non-aromatic carbocyclic group containing from 3 to 10 ring members, which may include fused, bridged or spiro ring systems. Non-limiting examples of fused bicyclic or bridged ring systems include bicyclo[1.1.1]pentane, bicyclo[2.1.1]hexane, bicyclo[2.2.1]heptane, bicyclo[3.1.1]heptane, bicyclo[3.2.1]octane, and bicyclo[2.2.2]octane. Non-limiting examples monocyclic C₃-C₈cycloalkyl groups include cyclopropyl, cyclobutyl, cyclopentyl, cyclohexyl, cycloheptyl and cyclooctyl groups.

The term “heterocycloalkyl” means any mono- or bi-cyclic non-aromatic carbocyclic group, composed of from 3 to 10 ring members, and containing from one to 3 hetero atoms selected from oxygen, sulphur, SO, SO₂ and nitrogen, it being understood that bicyclic group may be fused or spiro type. C₃-C₈heterocycloalkyl refers to heterocycloalkyl having 3 to 8 ring carbon atoms. The heterocycloalkyl can have 4 to 10 ring members.

The term heteroarylene, cycloalkylene, heterocycloalkylene mean a divalent heteroaryl, cycloalkyl and heterocycloalkyl.

The term “haloalkyl,” as used herein, refers to a linear or branched alkyl chain substituted with one or more halogen groups in place of hydrogens along the hydrocarbon chain. Examples of halogen groups suitable for substitution in the haloalkyl group include Fluorine, Bromine, Chlorine, and Iodine. Haloalkyl groups may include substitution with multiple halogen groups in place of hydrogens in an alkyl chain, wherein said halogen groups can be attached to the same carbon or to another carbon in the alkyl chain.

As used herein, the alkyl, alkenyl, alkynyl, alkoxy, amino, aryl, heteroaryl, cycloalkyl, and heterocycloalkyl groups may be optionally substituted by 1 to 4 groups selected from optionally substituted linear or branched (C₁-C₆)alkyl, optionally substituted linear or branched (C₂-C₆)alkenyl group, optionally substituted linear or branched (C₂-C₆)alkynyl group, optionally substituted linear or branched (C₁-C₆)alkoxy, optionally substituted (C₁-C₆)alkyl-S—, hydroxy, oxo (or N-oxide where appropriate), nitro, cyano, —C(O)—OR₀′, —O—C(O)—R₀′, —C(O)—NR₀′R₀″, —NR₀′R₀″, (C═NR₀′)—OR₀″, linear or branched (C₁-C₆) haloalkyl, trifluoromethoxy, or halogen, wherein R₀′ and R₀″ are each independently a hydrogen atom or an optionally substituted linear or branched (C₁-C₆)alkyl group, and wherein one or more of the carbon atoms of linear or branched (C₁-C₆)alkyl group is optionally deuterated.

The term “polyoxyethylene”, “polyethylene glycol” or “PEG”, as used herein, refers to a linear chain, a branched chain or a star shaped configuration comprised of (OCH₂CH₂) groups. In certain embodiments a polyethylene or PEG group is —(OCH₂CH₂)ₜ*—, where t is 1-40 or 4-40, and where the “—” indicates the end directed toward the self-immolative spacer and the “*—” indicates the point of attachment to a terminal end group R′ where R′ is OH, OCH₃ or OCH₂CH₂C(═O)OH. In other embodiments a polyethylene or PEG group is —(CH₂CH₂O)ₜ*—, where t is 1-40 or 4-40, and where the “—” indicates the end directed toward the self-immolative spacer and the “*—” indicates the point of attachment to a terminal end group R″ where R″ is H, CH₃ or CH₂CH₂C(═O)OH. For example, the term “PEG12” as used herein means that t is 12.

The term “polyalkylene glycol”, as used herein, refers to a linear chain, a branched chain or a star shaped configuration comprised of (O(CH₂)ₘ)ₙ groups. In certain embodiments a polyethylene or PEG group is —(O(CH₂)ₘ)ₜ*—, where m is 1-10, t is 1-40 or 4-40, and where the “—” indicates the end directed toward the self-immolative spacer and the “*—” indicates the point of attachment to a terminal end group R′ where R′ is OH, OCH₃ or OCH₂CH₂C(═O)OH. In other embodiments a polyethylene or PEG group is —((CH₂)ₘO)ₜ*—, where m is 1-10, t is 1-40 or 4-40, and where the “—” indicates the end directed toward the self-immolative spacer and the “*—” indicates the point of attachment to a terminal end group R″ where R″ is H, CH₃ or CH₂CH₂C(═O)OH.

The term “reactive group”, as used herein, is a functional group capable of forming a covalent bond with a functional group of an antibody, an antibody fragment, or another reactive group attached to an antibody or antibody fragment. Non limiting examples of such functional groups include reactive groups of Table 3 provided herein.

The term “attachment group” or “coupling group”, as used herein, refers to a bivalent moiety which links the bridging spacer to the antibody or fragment thereof. The attachment or coupling group is a bivalent moiety formed by the reaction between a reaction group and a functional group on the antibody or fragment thereof. Non limiting examples of such bivalent moieties include the bivalent chemical moieties given in Table 3 and Table 4 provided herein.

The term “bridging spacer”, as used herein, refers to one or more linker components which are covalently attached together to form a bivalent moiety which links the bivalent peptide spacer to the reactive group, links the bivalent peptide space to the coupling group, or links the attachment group to the at least one cleavable group. In certain embodiments the “bridging spacer” comprises a carboxyl group attached to the N-terminus of the bivalent peptide spacer via an amide bond.

The term “spacer moiety”, as used herein, refers to one or more linker components which are covalently attached together to form a moiety which links the self-immolative spacer to the hydrophilic moiety.

The term “bivalent peptide spacer”, as used herein, refers to bivalent linker comprising one or more amino acid residues covalently attached together to form a moiety which links the bridging spacer to the self immolative spacer. The one or more amino acid residues can be an residue of amino acids selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine.

In certain embodiments a “bivalent peptide spacer” is a combination of 2 to four amino acid residues where each residue is independently selected from a residue of an amino acid selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu),methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine, for example -ValCit*; -CitVal*; -AlaAla*; -AlaCit*; -CitAla*; -AsnCit*; -CitAsn*; -CitCit*; -ValGlu*; -GluVal*; -SerCit*; -CitSer*; -LysCit*; -CitLys*; -AspCit*; -CitAsp*; -AlaVal*; -ValAla*; -PheAla*; -AlaPhe*; -PheLys*; -LysPhe*; -ValLys*; -LysVal*; -AlaLys*; -LysAla*; -PheCit*; -CitPhe*; -LeuCit*; -CitLeu*; -IleCit*; -CitIle*; -PheArg*; -ArgPhe*; -CitTrp*; -TrpCit*; -PhePheLys*; -LysPhePhe*; -DPhePheLys*; -DLysPhePhe*; -GlyPheLys*; -LysPheGly*; -GlyPheLeuGly- [SEQ ID NO: 62]; -GlyLeuPheGly- [SEQ ID NO: 63]; -AlaLeuAlaLeu-[SEQ ID NO: 64], -GlyGlyGly*; -GlyGlyGlyGly- [SEQ ID NO: 65]; -GlyPheValGly-[SEQ ID NO: 66]; and -GlyValPheGly- [SEQ ID NO: 67], where the “—” indicates the point of attachment to the bridging spacer and the “*” indicates the point of attachment to the self-immolative spacer.

The term “linker component”, as used herein, refers to a chemical moiety that is a part of the linker. Examples of linker components include: an alkylene group: —(CH₂)ₙ— which can either be linear or branched (where in this instance n is 1-18); an alkenylene group; an alkynylene group; an alkenyl group; an alkynyl group; an ethylene glycol unit: —OCH₂CH₂— or —CH₂CH₂O—; an polyethylene glycol unit: (—CH₂CH₂O—)ₓ (where x in this instance is 2-20); —O—; —S—; a carbonyl: —C(═O); an ester: C(═O)—O or O—C(═O); a carbonate: —OC(═O)O—; an amine: —NH—; an tertiary amine; an amide: —C(═O)—NH—, —NH—C(═O)— or —C(═O)N(C₁₋₆alkyl); a carbamate: —OC(═O)NH— or —NHC(═O)O; a urea: —NHC(═O)NH; a sulfonamide: —S(O)₂NH— or —NHS(O)₂; an ether: —CH₂O— or —OCH₂—; an alkylene substituted with one or more groups independently selected from carboxy, sulfonate, hydroxyl, amine, amino acid, saccharide, phosphate and phosphonate); an alkenylene substituted with one or more groups independently selected from carboxy, sulfonate, hydroxyl, amine, amino acid, saccharide, phosphate and phosphonate); an alkynylene substituted with one or more groups independently selected from carboxy, sulfonate, hydroxyl, amine, amino acid, saccharide, phosphate and phosphonate); a C₁-C₁₀alkylene in which one or more methylene groups is replace by one or more —S—, —NH— or —O— moieties; a ring systems having two available points of attachment such as a divalent ring selected from phenyl (including 1,2- 1,3- and 1,4-di-substituted phenyls), a C₅-C₆ heteroaryl, a C₃-C₈ cycloalkyl (including 1,1-disubstituted cyclopropyl, cyclobutyl, cyclopentyl or cyclohexyl, and 1,4-disubstituted cyclohexyl), and a C₄-C₈ heterocycloalkyl; a residue of an amino acid selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu),methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine; a combination of 2 or more amino acid residues where each residue is independently selected from a residue of an amino acid selected from alanine (Ala), cysteine (Cys), aspartic acid (Asp), glutamic acid (Glu), phenylalanine (Phe), glycine (Gly), histidine (His), isoleucine (Ile), lysine (Lys), leucine (Leu),methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine, for example Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit; and a self-immolative spacer, wherein the self-immolative spacer comprises one or more protecting (triggering) groups which are susceptible to acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage.

Non-limiting examples of such self-immolative spacers include:

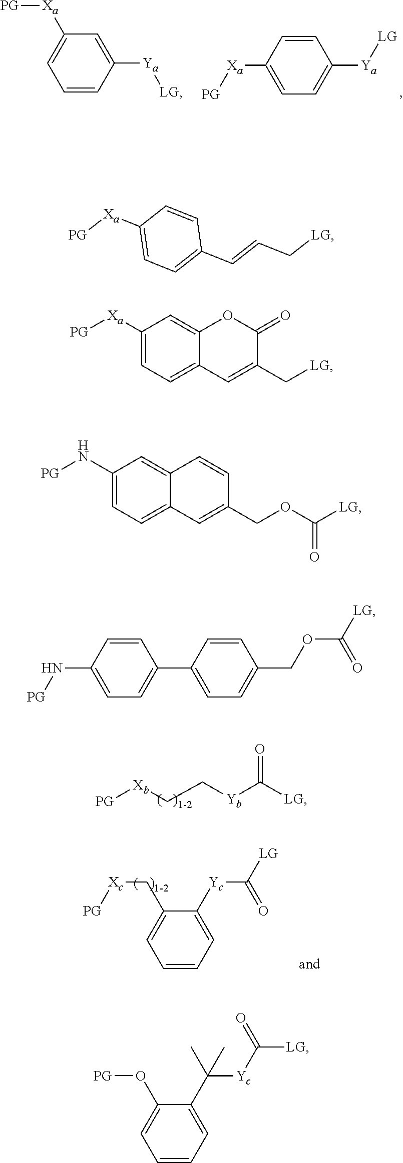

where:
PG is a protecting (triggering) group;
Xₐ is O, NH or S;
X_{b} is O, NH, NCH₃ or S;
X_{c} is O or NH;
Yₐ is CH₂, CH₂O or CH₂NH;
Y_{b} is CH₂, O or NH;
Y_{c} is a bond, CH₂, O or NH, and
LG is a leaving group such as a Drug moiety (D) of the Linker-Drug group of the invention.

Additional non-limiting examples of such self-immolative spacers are described in Angew. Chem. Int. Ed. 2015, 54, 7492-7509.

In addition, a linker component can be a chemical moiety which is readily formed by reaction between two reactive groups. Non-limiting examples of such chemical moieties are given in Table 3.

TABLE 3

| Reactive Group | Reactive Group | |
|---|---|---|
| 1 | 2 | Chemical |
| (RG1) | (RG2) | Moiety |
| a thiol | a thiol | —S—S— |
| a thiol | a maleimide | 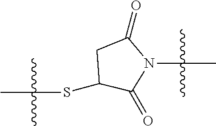 [SMILES: *SC1CC(=O)N(*)C1=O] |
| a thiol | a haloacetamide | 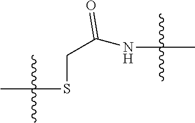 [SMILES: *NC(=O)CS*] |
| an azide | an alkyne | 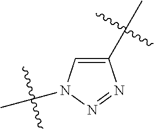 [SMILES: *c1cn(*)nn1] |
| | | or |
| | | 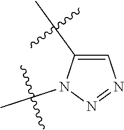 [SMILES: *c1cnnn1*] |
| an azide | a triaryl phosphine | 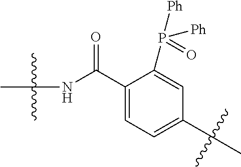 [SMILES: *NC(=O)c1ccc(*)cc1P(=O)(c1ccccc1)c1ccccc1] |
| an azide | a cyclooctyne | 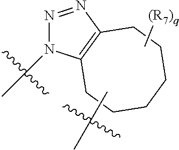 |
| | | or |
| | | 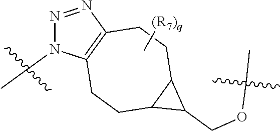 |
| | | or |
| | | 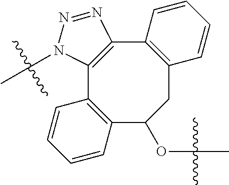 [SMILES: *OC1Cc2ccccc2-c2nnn(*)c2-c2ccccc21] |
| an azide | an oxanobornadiene | 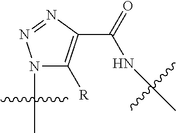 |
| a triaryl phosphine | an azide | 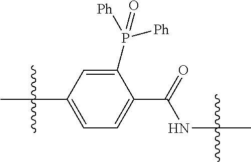 [SMILES: *NC(=O)c1ccc(*)cc1P(=O)(c1ccccc1)c1ccccc1] |
| an oxanobornadiene | an azide | 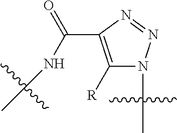 |
| an alkyne | an azide | 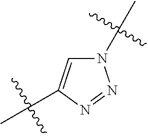 [SMILES: *c1cn(*)nn1] |
| | | or |
| | | 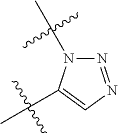 [SMILES: *c1cnnn1*] |
| a cyclooctyne | azide | 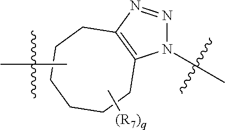 |
| | | or |
| | | 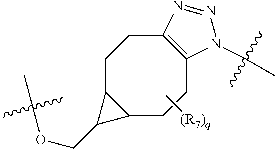 |
| | | or |
| | | 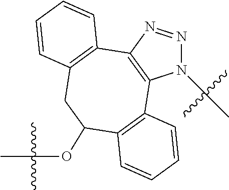 [SMILES: *OC1Cc2ccccc2-c2nnn(*)c2-c2ccccc21] |
| a cyclooctene | a diaryl tetrazine | 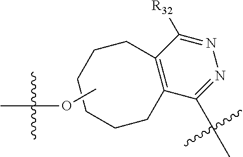 |
| | | or |
| | | 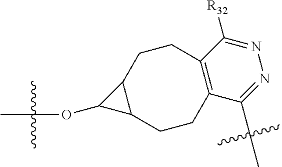 |
| a diaryl tetrazine | a cyclooctene | 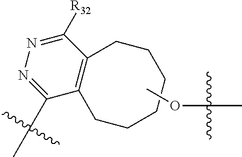 |
| | | or |
| | | 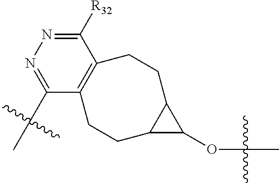 |
| a monoaryl tetrazine | a norbornene | 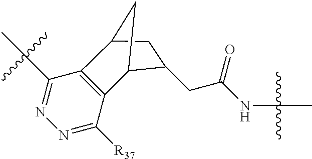 |
| a norbornene | a monoaryl tetrazine | 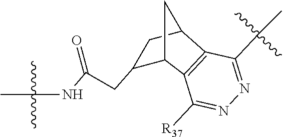 |
| an aldehyde | a hydroxylamine | 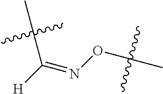 [SMILES: */C=N\O*] |
| an aldehyde | a hydrazine | 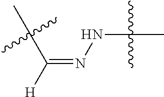 [SMILES: */C=N\N*] |
| an aldehyde | NH₂—NH—C(═O)— | 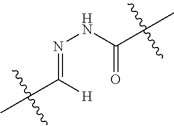 [SMILES: */C=N/NC(*)=O] |
| a ketone | a hydroxylamine | 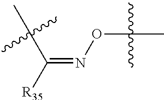 |
| a ketone | a hydrazine | 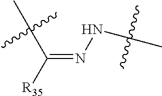 |
| a ketone | NH₂—NH—C(═O)— | 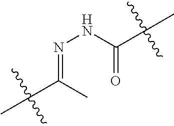 [SMILES: *C(=O)N/N=C(/*)C] |
| a hydroxylamine | an aldehyde | 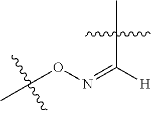 [SMILES: */C=N\O*] |
| a hydroxylamine | a ketone | 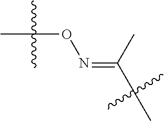 [SMILES: *O/N=C(/*)C] |
| a hydrazine | an aldehyde | 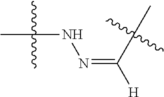 [SMILES: */C=N\N*] |
| a hydrazine | a ketone | 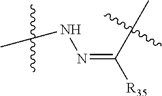 |
| NH₂—NH—C(═O)— | an aldehyde | 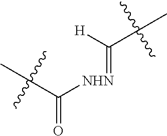 |
| NH_{2—}NH—C(═O)— | a ketone | 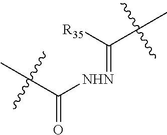 |
| a haloacetamide | a thiol | 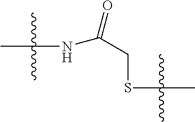 [SMILES: *NC(=O)CS*] |
| a maleimide | a thiol | 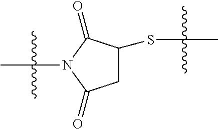 [SMILES: *SC1CC(=O)N(*)C1=O] |
| a vinyl sulfone | a thiol | 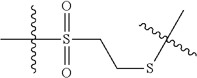 [SMILES: *SCCS(*)(=O)=O] |
| a thiol | a vinyl sulfone | 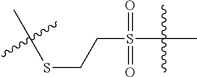 [SMILES: *SCCS(*)(=O)=O] |
| an aziridine | a thiol | 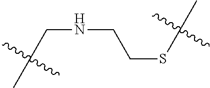 [SMILES: *CNCCS*] |
| | | or |
| | | 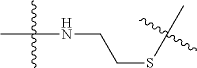 [SMILES: *NCCS*] |
| a thiol | an aziridine | 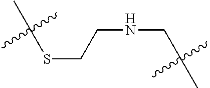 [SMILES: *CNCCS*] |
| | | or |
| | | 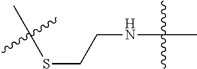 [SMILES: *NCCS*] |
| 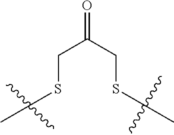 [SMILES: *SCC(=O)CS*] | hydroxylamine | 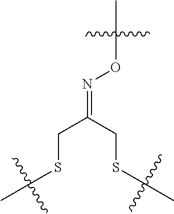 [SMILES: *ON=C(CS*)CS*] |
| 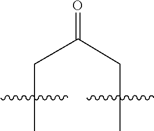 [SMILES: *CC(=O)C*] | hydroxylamine | 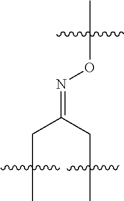 [SMILES: *CC(C*)=NO*] |
| 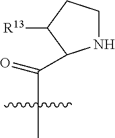 | 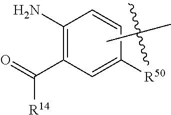 | 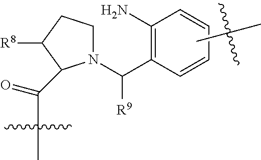 |
| 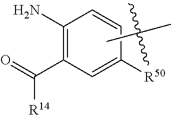 | 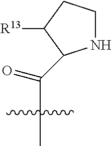 | 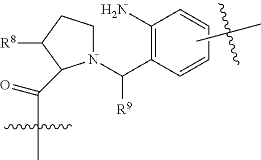 |
| 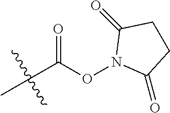 [SMILES: *C(=O)ON1C(=O)CCC1=O] | —NH₂, | amide |
| 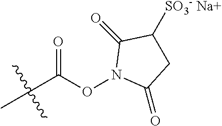 | | |
| 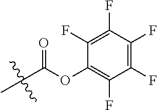 [SMILES: *C(=O)Oc1c(F)c(F)c(F)c(F)c1F] | | |
| 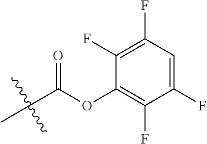 [SMILES: *C(=O)Oc1c(F)c(F)cc(F)c1F] | | |
| 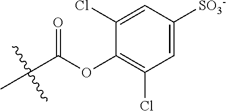 | | |
| —NH₂, | 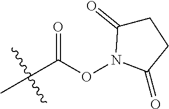 [SMILES: *C(=O)ON1C(=O)CCC1=O] | amide |
| | 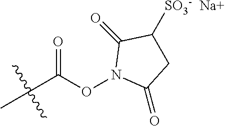 | |
| | 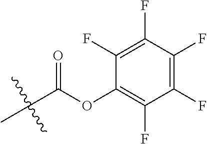 [SMILES: *C(=O)Oc1c(F)c(F)c(F)c(F)c1F] | |
| | 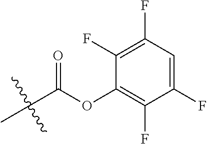 [SMILES: *C(=O)Oc1c(F)c(F)cc(F)c1F] | |
| | 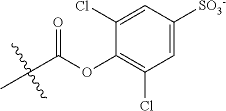 | |
| CoA or CoA analogue | Serine residue | 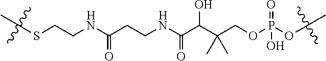 [SMILES: *OP(=O)(O)OCC(C)(C)C(O)C(=O)NCCC(=O)NCCS*] |
| | | 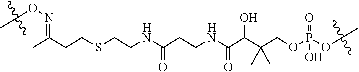 [SMILES: *O/N=C(\C)CCSCCNC(=O)CCNC(=O)C(O)C(C)(C)COP(=O)(O)O*] |
| | | 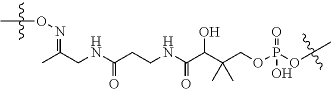 [SMILES: *O/N=C(\C)CNC(=O)CCNC(=O)C(O)C(C)(C)COP(=O)(O)O*] |
| | | 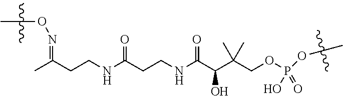 [SMILES: *O/N=C(\C)CCNC(=O)CCNC(=O)[C@H](O)C(C)(C)COP(=O)(O)O*] |
| | | 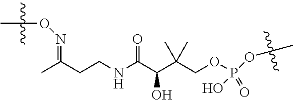 [SMILES: *O/N=C(\C)CCNC(=O)[C@H](O)C(C)(C)COP(=O)(O)O*] |
| | | 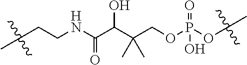 [SMILES: *CCNC(=O)C(O)C(C)(C)COP(=O)(O)O*] |
| | | 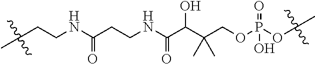 [SMILES: *CCNC(=O)CCNC(=O)C(O)C(C)(C)COP(=O)(O)O*] |
| | | 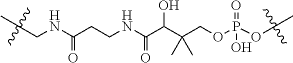 [SMILES: *CNC(=O)CCNC(=O)C(O)C(C)(C)COP(=O)(O)O*] |
| pyridyldithiol | thiol | Disulfide |

where: R³² in Table 3 is H, C₁₋₄ alkyl, phenyl, pyrimidine or pyridine; R³⁵ in Table 3 is H, C₁₋₆alkyl, phenyl or C₁₋₄alkyl substituted with 1 to 3-OH groups; each R⁷ in Table 3 is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH; R³⁷ in Table 3 is independently selected from H, phenyl and pyridine; q in Table 3 is 0, 1, 2 or 3; R⁸ and R¹³ in Table 3 is H or methyl; and R⁹ and R¹⁴ in Table 3 is H, —CH₃ or phenyl; R in Table 3 is H or any suitable substituent; and R⁵⁰ in Table 3 is H.

In addition, a linker component can be a group listed in Table 4 below.

TABLE 4

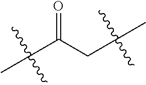 [SMILES: *CC(*)=O]

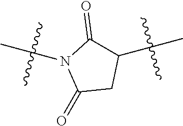 [SMILES: *C1CC(=O)N(*)C1=O]

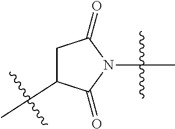 [SMILES: *C1CC(=O)N(*)C1=O]

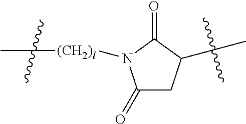

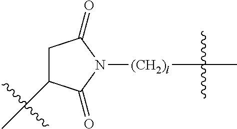

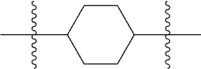 [SMILES: *C1CCC(*)CC1]

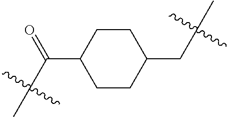 [SMILES: *CC1CCC(C(*)=O)CC1]

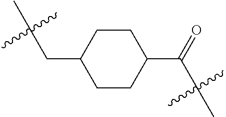 [SMILES: *CC1CCC(C(*)=O)CC1]

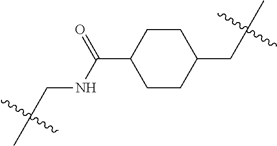 [SMILES: *CNC(=O)C1CCC(C*)CC1]

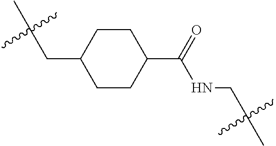 [SMILES: *CNC(=O)C1CCC(C*)CC1]

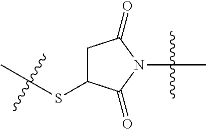 [SMILES: *SC1CC(=O)N(*)C1=O]

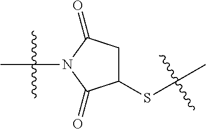 [SMILES: *SC1CC(=O)N(*)C1=O]

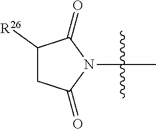

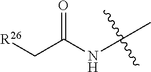

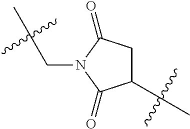 [SMILES: *CN1C(=O)CC(*)C1=O]

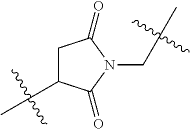 [SMILES: *CN1C(=O)CC(*)C1=O]

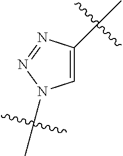 [SMILES: *c1cn(*)nn1]

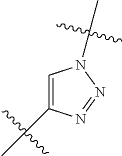 [SMILES: *c1cn(*)nn1]

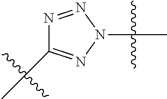 [SMILES: *c1nnn(*)n1]

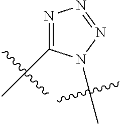 [SMILES: *c1nnnn1*]

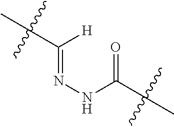 [SMILES: */C=N/NC(*)=O]

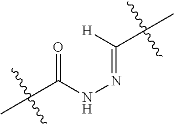 [SMILES: */C=N/NC(*)=O]

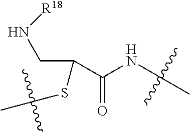

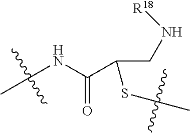

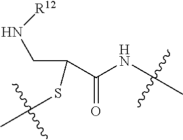

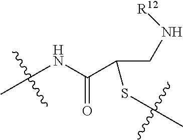

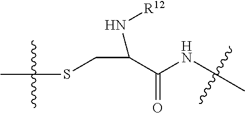

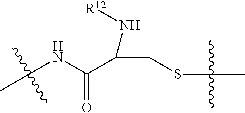

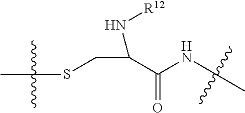

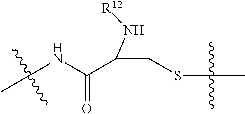

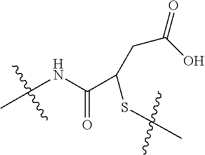 [SMILES: *NC(=O)C(CC(=O)O)S*]

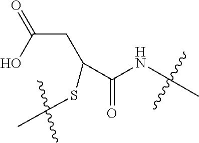 [SMILES: *NC(=O)C(CC(=O)O)S*]

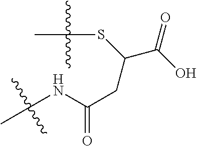 [SMILES: *NC(=O)CC(S*)C(=O)O]

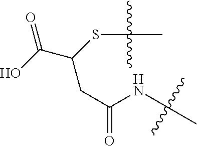 [SMILES: *NC(=O)CC(S*)C(=O)O]

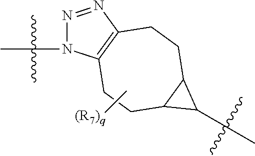

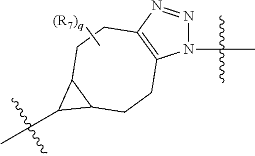

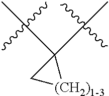 [SMILES: *C1(*)CC1]

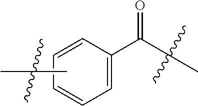

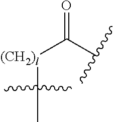

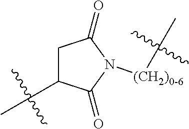 [SMILES: *CN1C(=O)CC(*)C1=O]

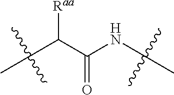

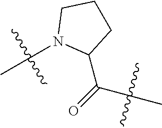 [SMILES: *C(=O)C1CCCN1*]

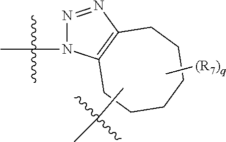

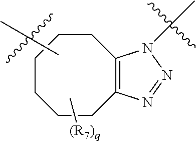

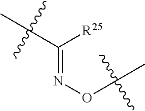

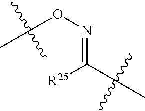

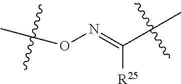

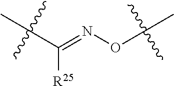

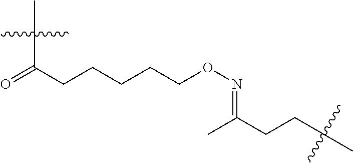 [SMILES: *CC/C(C)=N/OCCCCCC(*)=O]

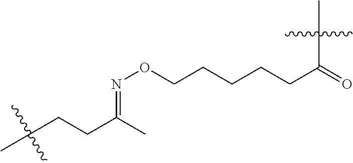 [SMILES: *CC/C(C)=N/OCCCCCC(*)=O]

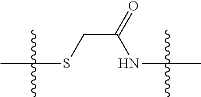 [SMILES: *NC(=O)CS*]

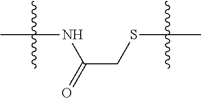 [SMILES: *NC(=O)CS*]

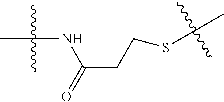 [SMILES: *NC(=O)CCS*]

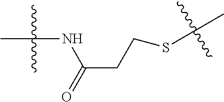 [SMILES: *NC(=O)CCS*]

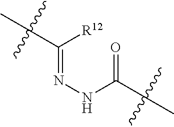

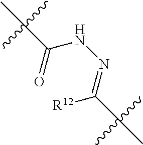

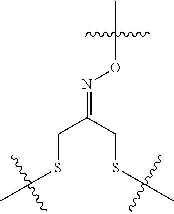 [SMILES: *ON=C(CS*)CS*]

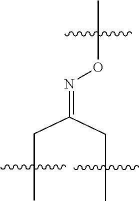 [SMILES: *CC(C*)=NO*]

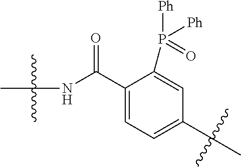 [SMILES: *NC(=O)c1ccc(*)cc1P(=O)(c1ccccc1)c1ccccc1]

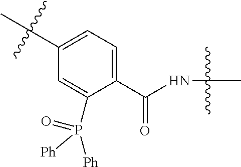 [SMILES: *NC(=O)c1ccc(*)cc1P(=O)(c1ccccc1)c1ccccc1]

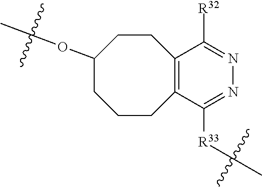

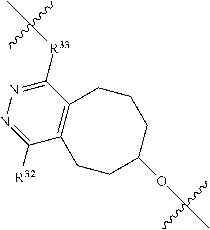

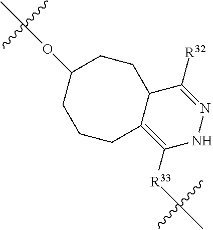

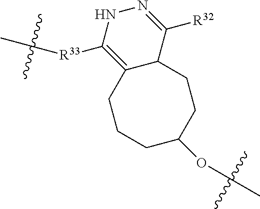

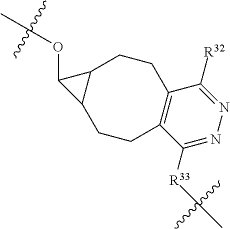

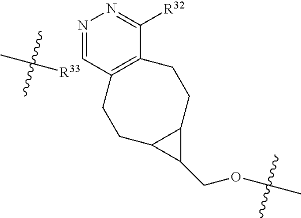

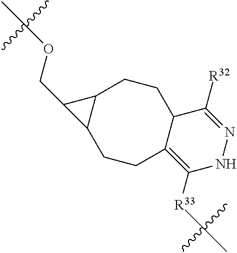

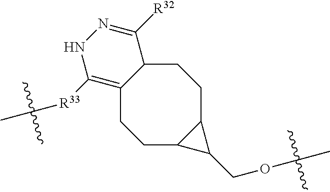

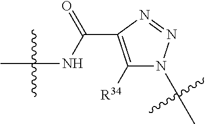

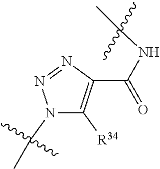

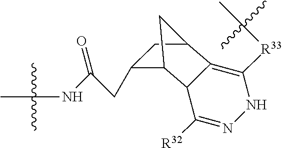

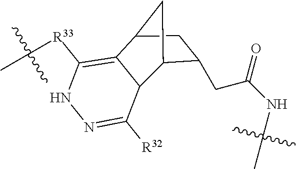

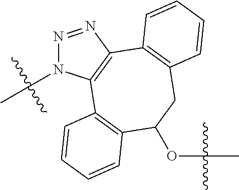 [SMILES: *OC1Cc2ccccc2-c2nnn(*)c2-c2ccccc21]

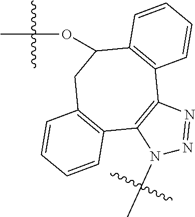 [SMILES: *OC1Cc2ccccc2-c2c(nnn2*)-c2ccccc21]

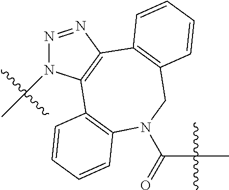 [SMILES: *C(=O)N1Cc2ccccc2-c2nnn(*)c2-c2ccccc21]

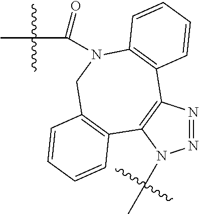 [SMILES: *C(=O)N1Cc2ccccc2-c2c(nnn2*)-c2ccccc21]

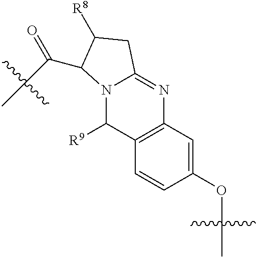

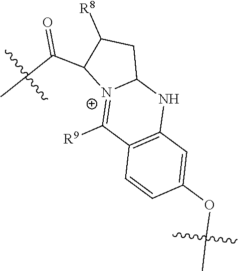

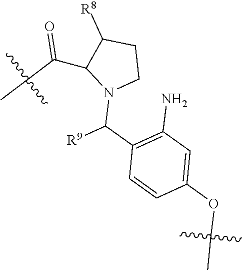

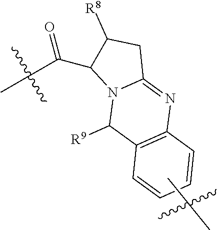

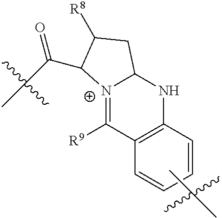

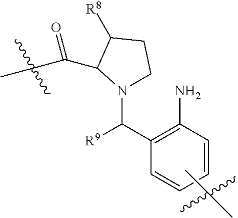

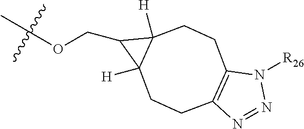

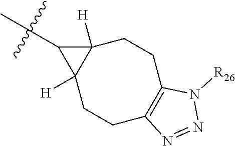

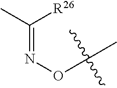

each R⁷ independently selected from H, C₁₋₆alkyl, fluoro, benzyloxy substituted with —C(═O)OH, benzyl substituted with —C(═O)OH, C₁₋₄ alkyl substituted with —C(═O)OH and C₁₋₄ alkyl substituted with —C(═O)OH;
each R¹² is independently selected from H and C₁—C₆alkyl
R⁸ is H or methyl;
R⁹ is H, —CH₃ or phenyl;
each R²⁵ is independently selected from H or C₁₋₄ alkyl;
each R¹⁸ is independently selected from a C₁—C₆alkyl, a C₁—C₆alkyl which is substituted with azido and a C₁—C₆alkyl which is substituted with 1 to 5 hydroxyl;
q is 0, 1, 2 or 3;
I is 1, 2, 3, 4, 5 or 6;
R²⁶ is 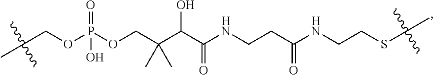 [SMILES: *COP(=O)(O)OCC(C)(C)C(O)C(=O)NCCC(=O)NCCS*] 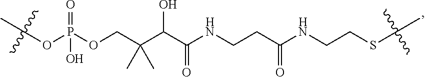 [SMILES: *OP(=O)(O)OCC(C)(C)C(O)C(=O)NCCC(=O)NCCS*] 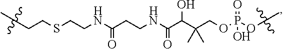 [SMILES: *CCSCCNC(=O)CCNC(=O)C(O)C(C)(C)COP(=O)(O)O*] 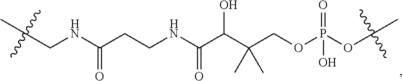 [SMILES: *CNC(=O)CCNC(=O)C(O)C(C)(C)COP(=O)(O)O*] 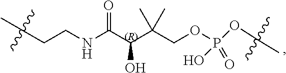 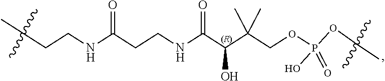 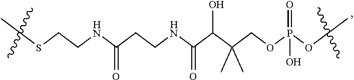 [SMILES: *OP(=O)(O)OCC(C)(C)C(O)C(=O)NCCC(=O)NCCS*] 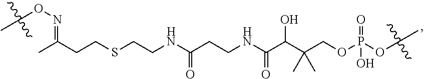 [SMILES: *O/N=C(\C)CCSCCNC(=O)CCNC(=O)C(O)C(C)(C)COP(=O)(O)O*] 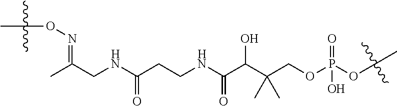 [SMILES: *O/N=C(\C)CNC(=O)CCNC(=O)C(O)C(C)(C)COP(=O)(O)O*] 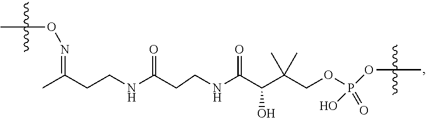 [SMILES: *O/N=C(\C)CCNC(=O)CCNC(=O)[C@@H](O)C(C)(C)COP(=O)(O)O*] 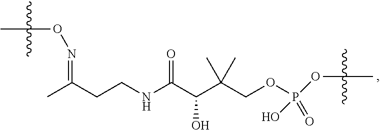 [SMILES: *O/N=C(\C)CCNC(=O)[C@@H](O)C(C)(C)COP(=O)(O)O*] 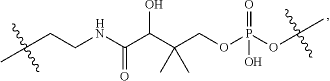 [SMILES: *CCNC(=O)C(O)C(C)(C)COP(=O)(O)O*] 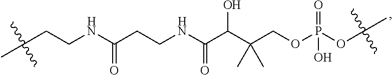 [SMILES: *CCNC(=O)CCNC(=O)C(O)C(C)(C)COP(=O)(O)O*]
or 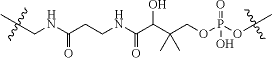 [SMILES: *CNC(=O)CCNC(=O)C(O)C(C)(C)COP(=O)(O)O*]
R³² is independently selected from H, C₁₋₄ alkyl, phenyl, pyrimidine and pyridine;
R³³ is independently selected from 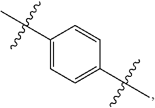 [SMILES: *c1ccc(*)cc1] 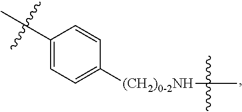 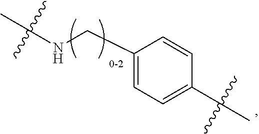 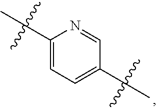 [SMILES: *c1ccc(*)nc1] 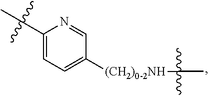 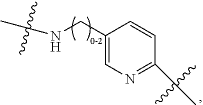 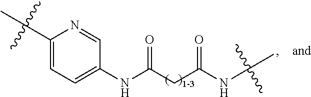 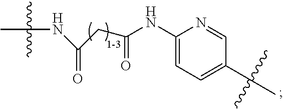
R³⁴ is independently selected from H, C₁₋₄ alkyl, and C₁₋₆ haloalkyl, and
R^{aa} is an amino acid side chain.

As used herein, when a partial structure of a compound is illustrated, a wavy line ( [symbol] ) indicates the point of attachment of the partial structure to the rest of the molecule.

The terms “self-immolative spacer” and “self-immolative group”, as used herein, refer a moiety comprising one or more triggering groups (TG) which are activated by acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage, and after activation the protecting group is removed, which generates a cascade of disassembling reactions leading to the temporally sequential release of a leaving group. Such cascade of reactions can be, but not limited to, 1,4-, 1,6- or 1,8-elimination reactions.

Non-limiting examples of self-immolative spacer or group include:

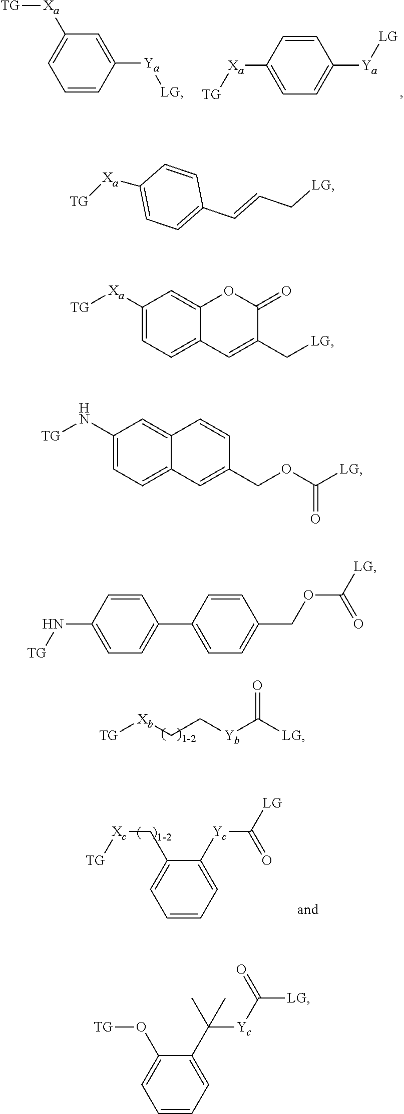

wherein such groups can be optionally substituted, and
wherein:
   TG is a triggering group;
   Xₐ is O, NH or S;
   X_{b} is O, NH, NCH₃ or S;
   X_{c} is O or NH;
   Yₐ is CH₂, CH₂O or CH₂NH;
   Y_{b} is CH₂, O or NH;
   Y_{c} is a bond, CH₂, O or NH, and
   LG is a leaving group such as a Drug moiety (D) of the Linker-Drug group of the invention.

Additional non-limiting examples of self-immolative spacers are described in Angew. Chem. Int. Ed. 2015, 54, 7492-7509.

In certain embodiment the self-immolative spacer is moiety having the structure

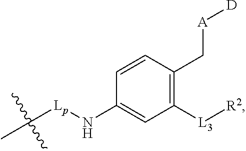

where Lp is an enzymatically cleavable bivalent peptide spacer and A, D, L₃ and R² are as defined herein.

In preferred embodiments, the self-immolative spacer is moiety having the structure

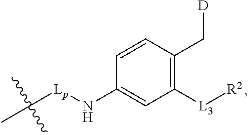

where Lp is an enzymatically cleavable bivalent peptide spacer and D, L₃ and R² are as defined herein. In some embodiments, D is a quaternized tertiary amine-containing Bcl-xL inhibitor.

In other preferred embodiments, the self-immolative spacer is moiety having the structure

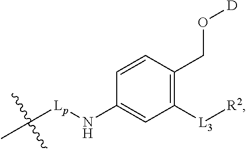

where Lp is an enzymatically cleavable bivalent peptide spacer and D, L₃ and R² are as defined herein.

The term “hydrophilic moiety”, as used herein, refers to moiety that is has hydrophilic properties which increases the aqueous solubility of the Drug moiety (D) when the Drug moiety (D) is attached to the linker group of the invention. Examples of such hydrophilic groups include, but are not limited to, polyethylene glycols, polyalkylene glycols, sugars, oligosaccharides, polypeptides a C₂-C₆alkyl substituted with 1 to 3

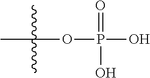 [SMILES: *OP(=O)(O)O]

groups.

### Drug Moieties

In some embodiments, an intermediate, which is the precursor of the linker moiety, is reacted with the drug moiety (e.g., the Bcl-xL inhibitor) under appropriate conditions. In some embodiments, reactive groups are used on the drug and/or the intermediate or linker. The product of the reaction between the drug and the intermediate, or the derivatized drug (drug plus linker), is subsequently reacted with the antibody or antigen-binding fragment under conditions that facilitate conjugation of the drug and intermediate or derivatized drug and antibody or antigen-binding fragment. Alternatively, the intermediate or linker may first be reacted with the antibody or antigen-binding fragment, or a derivatized antibody or antigen-binding fragment, and then reacted with the drug or derivatized drug.

A number of different reactions are available for covalent attachment of the drug moiety and/or linker moiety to the antibody or antigen-binding fragment. This is often accomplished by reaction of one or more amino acid residues of the antibody or antigen-binding fragment, including the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a drug moiety to an amino (or carboxy) group on an antibody or antigen-binding fragment. Additionally, bifunctional agents such as dialdehydes or imidoesters may also be used to link the amino group on a drug moiety to an amino group on an antibody or antigen-binding fragment. Also available for attachment of drugs (e.g., a Bcl-xL inhibitor) to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure. Examples of drug moieties that can be generated and linked to an antibody or antigen-binding fragment using various chemistries known to in the art include Bcl-xL inhibitors, e.g., the Bcl-xL inhibitors described and exemplified herein.

Suitable drug moieties may comprise a compound of the formulas (I), (IA), (IB), (IC), (II), (IIA), (IIB) or (IIC) or an enantiomer, diastereoisomer, and/or addition salt thereof with a pharmaceutically acceptable acid or base. Additionally, the drug moiety may comprise any compounds of the Bcl-xL inhibitor (D) described herein.

In some embodiments, the drug moiety (D) comprises a formula selected from Table A2.

In some embodiments, the drug moiety (D) comprises a Bcl-XL inhibitor known in the art, for example, ABT-737 and ABT-263.

In some embodiments, the drug moiety (D) comprises a Bcl-xL inhibitor selected from:

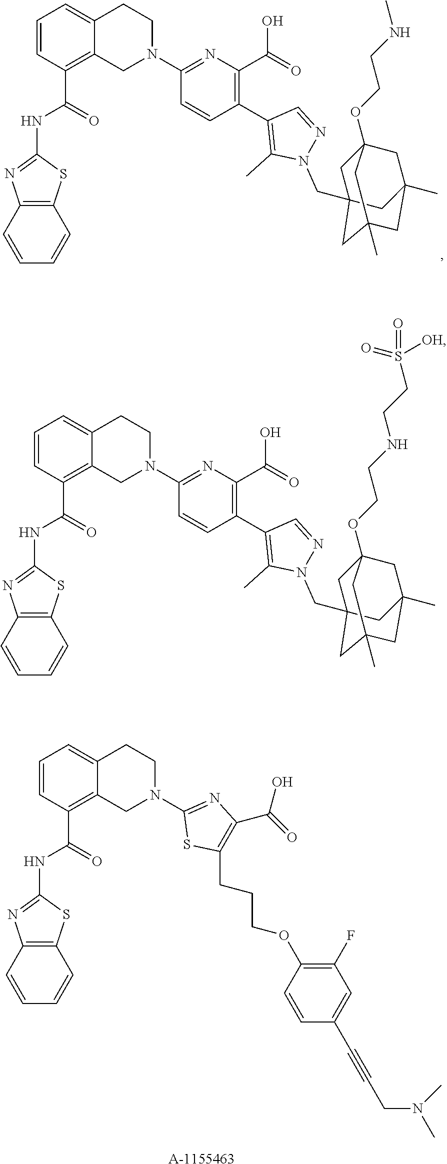 [SMILES: CNCCOC12CC3(C)CC(C)(CC(Cn4ncc(-c5ccc(N6CCc7cccc(C(=O)Nc8nc9ccccc9s8)c7C6)nc5C(=O)O)c4C)(C3)C1)C2 ; Cc1c(-c2ccc(N3CCc4cccc(C(=O)Nc5nc6ccccc6s5)c4C3)nc2C(=O)O)cnn1CC12CC3(C)CC(C)(C1)CC(OCCNCCS(=O)(=O)O)(C3)C2 ; CN(C)CC#Cc1ccc(OCCCc2sc(N3CCc4cccc(C(=O)Nc5nc6ccccc6s5)c4C3)nc2C(=O)O)c(F)c1 ; "A-1155463"]

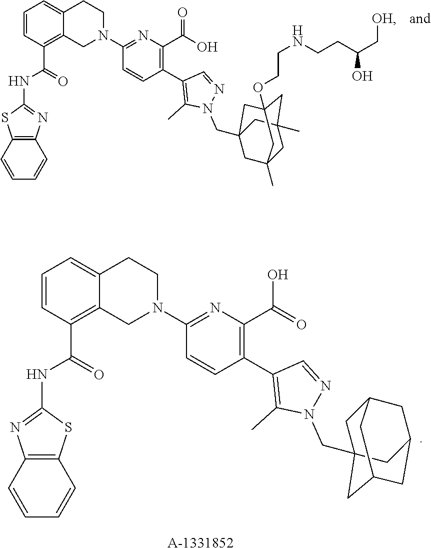 [SMILES: Cc1c(-c2ccc(N3CCc4cccc(C(=O)Nc5nc6ccccc6s5)c4C3)nc2C(=O)O)cnn1CC12CC3(C)CC(C)(C1)CC(OCCNCC[C@H](O)CO)(C3)C2 ; Cc1c(-c2ccc(N3CCc4cccc(C(=O)Nc5nc6ccccc6s5)c4C3)nc2C(=O)O)cnn1CC12CC3CC(CC(C3)C1)C2 ; "A-1331852"]

In some embodiments, the linker-drug (or “linker-payload”) moiety-(L-D) may comprise a compounds in Table B or an enantiomer, diastereoisomer, deuterated derivative, and/or a pharmaceutically acceptable salt of any of the foregoing.

### Drug Loading

Drug loading is represented by p, and is also referred to herein as the drug-to-antibody ratio (DAR). Drug loading may range from 1 to 16 drug moieties per antibody or antigen-binding fragment. In some embodiments, p is an integer from 1 to 16. In some embodiments, p is an integer from 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p is an integer from 2 to 10, 2 to 9, 2 to 8, 2 to 7, 2 to 6, 2 to 5, 2 to 4, or 2 to 3. In some embodiments, p is an integer from 1 to 16. In some embodiments, p is an integer from 1 to 8. In some embodiments, p is an integer from 1 to 5. In some embodiments, p is an integer from 2 to 4. In some embodiments, p is 1, 2, 3, 4, 5, 6, 7, or 8. In some embodiments, p is 2. In some embodiments, p is 4.

Drug loading may be limited by the number of attachment sites on the antibody or antigen-binding fragment. In some embodiments, the linker moiety (L) of the ADC attaches to the antibody or antigen-binding fragment through a chemically active group on one or more amino acid residues on the antibody or antigen-binding fragment. For example, the linker may be attached to the antibody or antigen-binding fragment via a free amino, imino, hydroxyl, thiol, or carboxyl group (e.g., to the N- or C-terminus, to the epsilon amino group of one or more lysine residues, to the free carboxylic acid group of one or more glutamic acid or aspartic acid residues, or to the sulfhydryl group of one or more cysteine residues). The site to which the linker is attached can be a natural residue in the amino acid sequence of the antibody or antigen-binding fragment, or it can be introduced into the antibody or antigen-binding fragment, e.g., by DNA recombinant technology (e.g., by introducing a cysteine residue into the amino acid sequence) or by protein biochemistry (e.g., by reduction, pH adjustment, or hydrolysis).

In some embodiments, the number of drug moieties that can be conjugated to an antibody or antigen-binding fragment is limited by the number of free cysteine residues. For example, where the attachment is a cysteine thiol group, an antibody may have only one or a few cysteine thiol groups, or may have only one or a few sufficiently reactive thiol groups through which a linker may be attached. Generally, antibodies do not contain many free and reactive cysteine thiol groups that may be linked to a drug moiety. Indeed, most cysteine thiol residues in antibodies are involved in either interchain or intrachain disulfide bonds. Conjugation to cysteines can therefore, in some embodiments, require at least partial reduction of the antibody. Over-attachment of linker-toxin to an antibody may destabilize the antibody by reducing the cysteine residues available to form disulfide bonds. Therefore, an optimal drug: antibody ratio should increase potency of the ADC (by increasing the number of attached drug moieties per antibody) without destabilizing the antibody or antigen-binding fragment. In some embodiments, an optimal ratio may be 2, 4, 6, or 8. In some embodiments, an optimal ratio may be 2 or 4.

In some embodiments, an antibody or antigen-binding fragment is exposed to reducing conditions prior to conjugation in order to generate one or more free cysteine residues. An antibody, in some embodiments, may be reduced with a reducing agent such as dithiothreitol (DTT) or tris(2-carboxyethyl)phosphine (TCEP), under partial or total reducing conditions, to generate reactive cysteine thiol groups. Unpaired cysteines may be generated through partial reduction with limited molar equivalents of TCEP, which can reduce the interchain disulfide bonds which link the light chain and heavy chain (one pair per H-L pairing) and the two heavy chains in the hinge region (two pairs per H—H pairing in the case of human IgG1) while leaving the intrachain disulfide bonds intact (Stefano et al. (2013) Methods Mol Biol. 1045:145-71). In embodiments, disulfide bonds within the antibodies are reduced electrochemically, e.g., by employing a working electrode that applies an alternating reducing and oxidizing voltage. This approach can allow for on-line coupling of disulfide bond reduction to an analytical device (e.g., an electrochemical detection device, an NMR spectrometer, or a mass spectrometer) or a chemical separation device (e.g., a liquid chromatograph (e.g., an HPLC) or an electrophoresis device (see, e.g., US 2014/0069822)). In some embodiments, an antibody is subjected to denaturing conditions to reveal reactive nucleophilic groups on amino acid residues, such as cysteine.

The drug loading of an ADC may be controlled in different ways, e.g., by: (i) limiting the molar excess of drug-linker intermediate or linker reagent relative to antibody; (ii) limiting the conjugation reaction time or temperature; (iii) partial or limiting reductive conditions for cysteine thiol modification; and/or (iv) engineering by recombinant techniques the amino acid sequence of the antibody such that the number and position of cysteine residues is modified for control of the number and/or position of linker-drug attachments.

In some embodiments, free cysteine residues are introduced into the amino acid sequence of the antibody or antigen-binding fragment. For example, cysteine engineered antibodies can be prepared wherein one or more amino acids of a parent antibody are replaced with a cysteine amino acid. Any form of antibody may be so engineered, i.e. mutated. For example, a parent Fab antibody fragment may be engineered to form a cysteine engineered Fab referred to as a “ThioFab.” Similarly, a parent monoclonal antibody may be engineered to form a “ThioMab.” A single site mutation yields a single engineered cysteine residue in a ThioFab, whereas a single site mutation yields two engineered cysteine residues in a ThioMab, due to the dimeric nature of the IgG antibody. DNA encoding an amino acid sequence variant of the parent polypeptide can be prepared by a variety of methods known in the art (see, e.g., the methods described in WO 2006/034488). These methods include, but are not limited to, preparation by site-directed (or oligonucleotide-mediated) mutagenesis, PCR mutagenesis, and cassette mutagenesis of an earlier prepared DNA encoding the polypeptide. Variants of recombinant antibodies may also be constructed by restriction fragment manipulation or by overlap extension PCR with synthetic oligonucleotides. ADCs of Formula (1) include, but are not limited to, antibodies that have 1, 2, 3, or 4 engineered cysteine amino acids (Lyon et al. (2012) Methods Enzymol. 502:123-38). In some embodiments, one or more free cysteine residues are already present in an antibody or antigen-binding fragment, without the use of engineering, in which case the existing free cysteine residues may be used to conjugate the antibody or antigen-binding fragment to a drug moiety.

Where more than one nucleophilic group reacts with a drug-linker intermediate or a linker moiety reagent followed by drug moiety reagent, in a reaction mixture comprising multiple copies of the antibody or antigen-binding fragment and linker moiety, then the resulting product can be a mixture of ADC compounds with a distribution of one or more drug moieties attached to each copy of the antibody or antigen-binding fragment in the mixture. In some embodiments, the drug loading in a mixture of ADCs resulting from a conjugation reaction ranges from 1 to 16 drug moieties attached per antibody or antigen-binding fragment. The average number of drug moieties per antibody or antigen-binding fragment (i.e., the average drug loading, or average p) may be calculated by any conventional method known in the art, e.g., by mass spectrometry (e.g., liquid chromatography-mass spectrometry (LC-MS)) and/or high-performance liquid chromatography (e.g., HIC-HPLC). In some embodiments, the average number of drug moieties per antibody or antigen-binding fragment is determined by liquid chromatography-mass spectrometry (LC-MS). In some embodiments, the average number of drug moieties per antibody or antigen-binding fragment is from about 1.5 to about 3.5, about 2.5 to about 4.5, about 3.5 to about 5.5, about 4.5 to about 6.5, about 5.5 to about 7.5, about 6.5 to about 8.5, or about 7.5 to about 9.5. In some embodiments, the average number of drug moieties per antibody or antigen-binding fragment is from about 2 to about 4, about 3 to about 5, about 4 to about 6, about 5 to about 7, about 6 to about 8, about 7 to about 9, about 2 to about 8, or about 4 to about 8.

In some embodiments, the average number of drug moieties per antibody or antigen-binding fragment is about 2. In some embodiments, the average number of drug moieties per antibody or antigen-binding fragment is about 1.5, about 1.6, about 1.7, about 1.8, about 1.9, about 2, about 2.1, about 2.2, about 2.3, about 2.4, or about 2.5. In some embodiments, the average number of drug moieties per antibody or antigen-binding fragment is 2.

In some embodiments, the average number of drug moieties per antibody or antigen-binding fragment is about 4. In some embodiments, the average number of drug moieties per antibody or antigen-binding fragment is about 3.5, about 3.6, about 3.7, about 3.8, about 3.9, about 4, about 4.1, about 4.2, about 4.3, about 4.4, or about 4.5. In some embodiments, the average number of drug moieties per antibody or antigen-binding fragment is 4.

In some embodiments, the term “about,” as used with respect to the average number of drug moieties per antibody or antigen-binding fragment, means plus or minus 20%, 15%, 10%, 5%, or 1%. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

Individual ADC compounds, or “species,” may be identified in the mixture by mass spectroscopy and separated by, e.g., UPLC or HPLC, e.g. hydrophobic interaction chromatography (HIC-HPLC). In some embodiments, a homogeneous or nearly homogenous ADC product with a single loading value may be isolated from the conjugation mixture, e.g., by electrophoresis or chromatography.

In some embodiments, higher drug loading (e.g., p>16) may cause aggregation, insolubility, toxicity, or loss of cellular permeability of certain antibody-drug conjugates. Higher drug loading may also negatively affect the pharmacokinetics (e.g., clearance) of certain ADCs. In some embodiments, lower drug loading (e.g., p<2) may reduce the potency of certain ADCs against target-expressing cells. In some embodiments, the drug loading for an ADC of the present disclosure ranges from about 2 to about 16, about 2 to about 10, about 2 to about 8; from about 2 to about 6; from about 2 to about 5; from about 3 to about 5; from about 2 to about 4; or from about 4 to about 8.

In some embodiments, a drug loading and/or an average drug loading of about 2 is achieved, e.g., using partial reduction of intrachain disulfides on the antibody or antigen-binding fragment, and provides beneficial properties. In some embodiments, a drug loading and/or an average drug loading of about 4 or about 6 or about 8 is achieved, e.g., using partial reduction of intrachain disulfides on the antibody or antigen-binding fragment, and provides beneficial properties. In some embodiments, a drug loading and/or an average drug loading of less than about 2 may result in an unacceptably high level of unconjugated antibody species, which can compete with the ADC for binding to a target antigen and/or provide for reduced treatment efficacy. In some embodiments, a drug loading and/or average drug loading of more than about 16 may result in an unacceptably high level of product heterogeneity and/or ADC aggregation. A drug loading and/or an average drug loading of more than about 16 may also affect stability of the ADC, due to loss of one or more chemical bonds required to stabilize the antibody or antigen-binding fragment.

The present disclosure includes methods of producing the described ADCs. Briefly, the ADCs comprise an antibody or antigen-binding fragment as the antibody or antigen-binding fragment, a drug moiety (e.g., a Bcl-XL inhibitor), and a linker that joins the drug moiety and the antibody or antigen-binding fragment. In some embodiments, the ADCs can be prepared using a linker having reactive functionalities for covalently attaching to the drug moiety and to the antibody or antigen-binding fragment. In some embodiments, the antibody or antigen-binding fragment is functionalized to prepare a functional group that is reactive with a linker or a drug-linker intermediate. For example, in some embodiments, a cysteine thiol of an antibody or antigen-binding fragment can form a bond with a reactive functional group of a linker or a drug-linker intermediate to make an ADC. In some embodiments, an antibody or antigen-binding fragment is prepared with bacterial transglutaminase (BTG)—reactive glutamines specifically functionalized with an amine containing cyclooctyne BCN(N-[(1R,8S,9s)-Bicyclo[6.1.0]non-4-yn-9-ylmethyloxycarbonyl]-1,8-diamino-3,6-dioxaoctane) moiety. In some embodiments, site-specific conjugation of a linker or a drug-linker intermediate to a BCN moiety of an antibody or antigen-binding fragment is performed, e.g., as described and exemplified herein. The generation of the ADCs can be accomplished by techniques known to the skilled artisan.

In some embodiments, an ADC is produced by contacting an antibody or antigen-binding fragment with a linker and a drug moiety (e.g., a Bcl-XL inhibitor) in a sequential manner, such that the antibody or antigen-binding fragment is covalently linked to the linker first, and then the pre-formed antibody-linker intermediate reacts with the drug moiety. The antibody-linker intermediate may or may not be subjected to a purification step prior to contacting the drug moiety. In other embodiments, an ADC is produced by contacting an antibody or antigen-binding fragment with a linker-drug compound pre-formed by reacting a linker with a drug moiety. The pre-formed linker-drug compound may or may not be subjected to a purification step prior to contacting the antibody or antigen-binding fragment. In other embodiments, the antibody or antigen-binding fragment contacts the linker and the drug moiety in one reaction mixture, allowing simultaneous formation of the covalent bonds between the antibody or antigen-binding fragment and the linker, and between the linker and the drug moiety. This method of producing ADCs may include a reaction, wherein the antibody or antigen-binding fragment contacts the antibody or antigen-binding fragment prior to the addition of the linker to the reaction mixture, and vice versa. In some embodiments, an ADC is produced by reacting an antibody or antigen-binding fragment with a linker joined to a drug moiety, such as a Bcl-XL inhibitor, under conditions that allow conjugation.

The ADCs prepared according to the methods described above may be subjected to a purification step. The purification step may involve any biochemical methods known in the art for purifying proteins, or any combination of methods thereof. These include, but are not limited to, tangential flow filtration (TFF), affinity chromatography, ion exchange chromatography, any charge or isoelectric point-based chromatography, mixed mode chromatography, e.g., CHT (ceramic hydroxyapatite), hydrophobic interaction chromatography, size exclusion chromatography, dialysis, filtration, selective precipitation, or any combination thereof.

### Therapeutic Uses and Compositions

Disclosed herein are methods of using the compositions described herein, e.g., the disclosed ADC compounds and compositions, in treating a subject for a disorder, e.g., a cancer. Compositions, e.g., ADCs, may be administered alone or in combination with at least one additional inactive and/or active agent, e.g., at least one additional therapeutic agent, and may be administered in any pharmaceutically acceptable formulation, dosage, and dosing regimen. Treatment efficacy may be evaluated for toxicity as well as indicators of efficacy and adjusted accordingly. Efficacy measures include, but are not limited to, a cytostatic and/or cytotoxic effect observed in vitro or in vivo, reduced tumor volume, tumor growth inhibition, and/or prolonged survival.

Methods of determining whether an ADC exerts a cytostatic and/or cytotoxic effect on a cell are known. For example, the cytotoxic or cytostatic activity of an ADC can be measured by, e.g., exposing mammalian cells expressing a target antigen of the ADC in a cell culture medium; culturing the cells for a period from about 6 hours to about 6 days; and measuring cell viability (e.g., using a CellTiter-Glo® (CTG) or MTT cell viability assay). Cell-based in vitro assays may also be used to measure viability (proliferation), cytotoxicity, and induction of apoptosis (caspase activation) of the ADC.

For determining cytotoxicity, necrosis or apoptosis (programmed cell death) may be measured. Necrosis is typically accompanied by increased permeability of the plasma membrane, swelling of the cell, and rupture of the plasma membrane. Apoptosis can be quantitated, for example, by measuring DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica (1999)2:34-7 (Roche Molecular Biochemicals).

Apoptosis may also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring apoptotic cell number has been described by Duke and Cohen, Current Protocols in Immunology (Coligan et al., eds. (1992) pp. 3.17.1-3.17.16). Cells also can be labeled with a DNA dye (e.g., acridine orange, ethidium bromide, or propidium iodide) and the cells observed for chromatin condensation and margination along the inner nuclear membrane. Apoptosis may also be determined, in some embodiments, by screening for caspase activity. In some embodiments, a Caspase-Glo® Assay can be used to measure activity of caspase-3 and caspase-7. In some embodiments, the assay provides a luminogenic caspase-3/7 substrate in a reagent optimized for caspase activity, luciferase activity, and cell lysis. In some embodiments, adding Caspase-Glo® 3/7 Reagent in an “add-mix-measure” format may result in cell lysis, followed by caspase cleavage of the substrate and generation of a “glow-type” luminescent signal, produced by luciferase. In some embodiments, luminescence may be proportional to the amount of caspase activity present, and can serve as an indicator of apoptosis. Other morphological changes that can be measured to determine apoptosis include, e.g., cytoplasmic condensation, increased membrane blebbing, and cellular shrinkage. Determination of any of these effects on cancer cells indicates that an ADC is useful in the treatment of cancers.

Cell viability may be measured, e.g., by determining in a cell the uptake of a dye such as neutral red, trypan blue, Crystal Violet, or ALAMAR™ blue (see, e.g., Page et al. (1993) Intl J Oncology 3:473-6). In such an assay, the cells are incubated in media containing the dye, the cells are washed, and the remaining dye, reflecting cellular uptake of the dye, is measured spectrophotometrically.

Cell viability may also be measured, e.g., by quantifying ATP, an indicator of metabolically active cells. In some embodiments, in vitro potency and/or cell viability of prepared ADCs or Bcl-xL inhibitor compounds may be assessed using a CellTiter-Glo® (CTG) cell viability assay, as described in the examples provided herein. In this assay, in some embodiments, the single reagent (CellTiter-Glo® Reagent) is added directly to cells cultured in serum-supplemented medium. The addition of reagent results in cell lysis and generation of a luminescent signal proportional to the amount of ATP present. The amount of ATP is directly proportional to the number of cells present in culture

Cell viability may also be measured, e.g., by measuring the reduction of tetrazolium salts. In some embodiments, in vitro potency and/or cell viability of prepared ADCs or Bcl-xL inhibitor compounds may be assessed using an MTT cell viability assay, as described in the examples provided herein. In this assay, in some embodiments, the yellow tetrazolium MTT (3-(4,5-dimethylthiazolyl-2)-2,5-diphenyltetrazolium bromide) is reduced by metabolically active cells, in part by the action of dehydrogenase enzymes, to generate reducing equivalents such as NADH and NADPH. The resulting intracellular purple formazan can then be solubilized and quantified by spectrophotometric means.

In certain aspects, the present disclosure features a method of killing, inhibiting or modulating the growth of a cancer cell or tissue by disrupting the expression and/or activity of Bcl-xL and/or one or more upstream modulators or downstream targets thereof. The method may be used with any subject where disruption of Bcl-xL expression and/or activity provides a therapeutic benefit. Subjects that may benefit from disrupting Bcl-xL expression and/or activity include, but are not limited to, those having or at risk of having a cancer such as a tumor or a hematological cancer. In some embodiments, the cancer is a breast cancer, multiple myeloma, plasma cell myeloma, leukemia, lymphoma, sarcoma, gastric cancer, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, or head and neck cancer. In some embodiments, the cancer is a lymphoma or gastric cancer.

In some embodiments, the disclosed ADCs may be administered in any cell or tissue that expresses EphA2, such as a EphA2-expressing cancer cell or tissue. An exemplary embodiment includes a method of killing a EphA2-expressing cancer cell or tissue. The method may be used with any cell or tissue that expresses EphA2, such as a cancerous cell or a metastatic lesion. Non-limiting examples of EphA2-expressing cancers include breast cancer, non-small cell lung cancer, pancreatic cancer, esophageal cancer, head and neck cancer, stomach cancer, bladder cancer, and colon cancer. Non-limiting examples of EphA2-expressing cells include EBC-1 cells and cells comprising a recombinant nucleic acid encoding EphA2 or a portion thereof.

Exemplary methods include the steps of contacting a cell with an ADC, as described herein, in an effective amount, i.e., an amount sufficient to kill the cell. The method can be used on cells in culture, e.g., in vitro, in vivo, ex vivo, or in situ. For example, cells that express EphA2 (e.g., cells collected by biopsy of a tumor or metastatic lesion; cells from an established cancer cell line; or recombinant cells), can be cultured in vitro in culture medium and the contacting step can be affected by adding the ADC to the culture medium. The method will result in killing of cells expressing EphA2, including in particular cancer cells expressing EphA2. Alternatively, the ADC can be administered to a subject by any suitable administration route (e.g., intravenous, subcutaneous, or direct contact with a tumor tissue) to have an effect in vivo.

The in vivo effect of a disclosed ADC therapeutic composition can be evaluated in a suitable animal model. For example, xenogeneic cancer models can be used, wherein cancer explants or passaged xenograft tissues are introduced into immune compromised animals, such as nude or SCID mice (Klein et al. (1997) Nature Med. 3:402-8). Efficacy may be predicted using assays that measure inhibition of tumor formation, tumor regression or metastasis, and the like.

In vivo assays that evaluate the promotion of tumor death by mechanisms such as apoptosis may also be used. In some embodiments, xenografts from tumor bearing mice treated with the therapeutic composition can be examined for the presence of apoptotic foci and compared to untreated control xenograft-bearing mice. The extent to which apoptotic foci are found in the tumors of the treated mice provides an indication of the therapeutic efficacy of the composition.

Further provided herein are methods of treating a disorder, e.g., a cancer. The compositions described herein, e.g., the ADCs disclosed herein, can be administered to a non-human mammal or human subject for therapeutic purposes. The therapeutic methods include administering to a subject having or suspected of having a cancer a therapeutically effective amount of a composition comprising an Bcl-xL inhibitor, e.g., an ADC where the inhibitor is linked to a targeting antibody that binds to an antigen (1) expressed on a cancer cell, (2) is accessible to binding, and/or (3) is localized or predominantly expressed on a cancer cell surface as compared to a non-cancer cell.

An exemplary embodiment is a method of treating a subject having or suspected of having a cancer, comprising administering to the subject a therapeutically effective amount of a composition disclosed herein, e.g., an ADC, composition, or pharmaceutical composition (e.g., any of the exemplary ADCs, compositions, or pharmaceutical compositions disclosed herein). In some embodiments, the cancer expresses a target antigen. In some embodiments, the cancer is a tumor or a hematological cancer. In some embodiments, the cancer is a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, pancreatic cancer, stomach cancer, colon cancer, or head and neck cancer. In some embodiments, the cancer is a lymphoma or gastric cancer. In some embodiments, the cancer is breast cancer, non-small cell lung cancer, pancreatic cancer, esophageal cancer, head and neck cancer, stomach cancer, bladder cancer, or colon cancer. In some embodiments, the cancer is breast cancer or non-small cell lung cancer.

Another exemplary embodiment is a method of delivering a Bcl-XL inhibitor to a cell expressing EphA2, comprising conjugating the Bcl-XL inhibitor to an antibody that immunospecifically binds to a EphA2 epitope and exposing the cell to the ADC. Exemplary cancer cells that express EphA2 for which the ADCs of the present disclosure are indicated include breast cancer cells, non-small cell lung cancer cells, pancreatic cancer cells, esophageal cancer cells, head and neck cancer cells, stomach cancer cells, bladder cancer cells, or colon cancer cells.

In certain aspects, the present disclosure further provides methods of reducing or inhibiting growth of a tumor (e.g., a EphA2-expressing tumor), comprising administering a therapeutically effective amount of an ADC or composition comprising an ADC. In some embodiments, the treatment is sufficient to reduce or inhibit the growth of the patient's tumor, reduce the number or size of metastatic lesions, reduce tumor load, reduce primary tumor load, reduce invasiveness, prolong survival time, and/or maintain or improve the quality of life. In some embodiments, the tumor is resistant or refractory to treatment with the antibody or antigen-binding fragment of the ADC (e.g., an anti-EphA2 antibody) when administered alone, and/or the tumor is resistant or refractory to treatment with the Bcl-XL inhibitor drug moiety when administered alone.

An exemplary embodiment is a method of reducing or inhibiting the growth of a tumor in a subject, comprising administering to the subject a therapeutically effective amount of an ADC, composition, or pharmaceutical composition (e.g., any of the exemplary ADCs, compositions, or pharmaceutical compositions disclosed herein). In some embodiments, the tumor expresses a target antigen. In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, pancreatic cancer, stomach cancer, colon cancer, or spleen cancer. In some embodiments, the tumor is a gastric cancer. In some embodiments, the tumor is a breast cancer, non-small cell lung cancer, pancreatic cancer, esophageal cancer, head and neck cancer, stomach cancer, bladder cancer, or colon cancer. In some embodiments, the tumor is a breast cancer or non-small cell lung cancer. In some embodiments, administration of the ADC, composition, or pharmaceutical composition reduces or inhibits the growth of the tumor by at least about 10%, at least about 20%, at least about 30%, at least about 40%, at least about 50%, at least about 60%, at least about 70%, at least about 80%, at least about 90%, at least about 95%, or at least about 99%, as compared to growth in the absence of treatment.

Another exemplary embodiment is a method of delaying or slowing the growth of a tumor in a subject, comprising administering to the subject a therapeutically effective amount of an ADC, composition, or pharmaceutical composition (e.g., any of the exemplary ADCs, compositions, or pharmaceutical compositions disclosed herein). In some embodiments, the tumor expresses a target antigen. In some embodiments, the tumor is a breast cancer, gastric cancer, bladder cancer, brain cancer, cervical cancer, colorectal cancer, esophageal cancer, hepatocellular cancer, melanoma, oral cancer, ovarian cancer, non-small cell lung cancer, prostate cancer, small cell lung cancer, or spleen cancer. In some embodiments, the tumor is a gastric cancer. In some embodiments, administration of the ADC, composition, or pharmaceutical composition delays or slows the growth of the tumor by at least about 10%, at least about 20%, at least about 30%, at least about 40%, at least about 50%, at least about 60%, at least about 70%, at least about 80%, at least about 90%, at least about 95%, or at least about 99%, as compared to growth in the absence of treatment.

In certain aspects, the present disclosure further provides methods of reducing or slowing the expansion of a cancer cell population (e.g., a EphA2-expressing cancer cell population), comprising administering a therapeutically effective amount of an ADC or composition comprising an ADC.

An exemplary embodiment is a method of reducing or slowing the expansion of a cancer cell population in a subject, comprising administering to the subject a therapeutically effective amount of an ADC, composition, or pharmaceutical composition (e.g., any of the exemplary ADCs, compositions, or pharmaceutical compositions disclosed herein). In some embodiments, the cancer cell population expresses a target antigen. In some embodiments, the cancer cell population is from a tumor or a hematological cancer. In some embodiments, the cancer cell population is from a breast cancer, multiple myeloma, plasma cell myeloma, leukemia, lymphoma, sarcoma, gastric cancer, acute myeloid leukemia, bladder cancer, brain cancer, bone marrow cancer, cervical cancer, chronic lymphocytic leukemia, colorectal cancer, esophageal cancer, hepatocellular cancer, lymphoblastic leukemia including acute lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, or head and neck cancer. In some embodiments, the cancer cell population is from a lymphoma or gastric cancer. In some embodiments, administration of the ADC, composition, or pharmaceutical composition reduces the cancer cell population by at least about 10%, at least about 20%, at least about 30%, at least about 40%, at least about 50%, at least about 60%, at least about 70%, at least about 80%, at least about 90%, at least about 95%, or at least about 99%, as compared to the population in the absence of treatment. In some embodiments, administration of the ADC, composition, or pharmaceutical composition slows the expansion of the cancer cell population by at least about 10%, at least about 20%, at least about 30%, at least about 40%, at least about 50%, at least about 60%, at least about 70%, at least about 80%, at least about 90%, at least about 95%, or at least about 99%, as compared to expansion in the absence of treatment.

Also provided herein are methods of determining whether a subject having or suspected of having a cancer will be responsive to treatment with the disclosed ADCs and compositions. An exemplary embodiment is a method of determining whether a subject having or suspected of having a cancer will be responsive to treatment with an ADC, composition, or pharmaceutical composition (e.g., any of the exemplary ADCs, compositions, or pharmaceutical compositions disclosed herein) by providing a biological sample from the subject; contacting the sample with the ADC; and detecting binding of the ADC to cancer cells in the sample. In some embodiments, the sample is a tissue biopsy sample, a blood sample, or a bone marrow sample. In some embodiments, the method comprises providing a biological sample from the subject; contacting the sample with the ADC; and detecting one or more markers of cancer cell death in the sample (e.g., increased expression of one or more apoptotic markers, reduced expansion of a cancer cell population in culture, etc.).

Further provided herein are therapeutic uses of the disclosed ADCs and compositions. An exemplary embodiment is an ADC, composition, or pharmaceutical composition (e.g., any of the exemplary ADCs, compositions, or pharmaceutical compositions disclosed herein) for use in treating a subject having or suspected of having a cancer (e.g., a EphA2-expressing cancer). Another exemplary embodiment is a use of an ADC, composition, or pharmaceutical composition (e.g., any of the exemplary ADCs, compositions, or pharmaceutical compositions disclosed herein) in treating a subject having or suspected of having a cancer (e.g., a EphA2-expressing cancer). Another exemplary embodiment is a use of an ADC, composition, or pharmaceutical composition (e.g., any of the exemplary ADCs, compositions, or pharmaceutical compositions disclosed herein) in a method of manufacturing a medicament for treating a subject having or suspected of having a cancer (e.g., a EphA2-expressing cancer). Methods for identifying subjects having cancers that express a target antigen (e.g., EphA2) are known in the art and may be used to identify suitable patients for treatment with a disclosed ADC compound or composition.

Moreover, ADCs of the present disclosure may be administered to a non-human mammal expressing an antigen with which the ADC is capable of binding for veterinary purposes or as an animal model of human disease. Regarding the latter, such animal models may be useful for evaluating the therapeutic efficacy of the disclosed ADCs (e.g., testing of dosages and time courses of administration).

The therapeutic compositions used in the practice of the foregoing methods may be formulated into pharmaceutical compositions comprising a pharmaceutically acceptable carrier suitable for the desired delivery method. An exemplary embodiment is a pharmaceutical composition comprising an ADC of the present disclosure and a pharmaceutically acceptable carrier, e.g., one suitable for a chosen means of administration, e.g., intravenous administration. The pharmaceutical composition may also comprise one or more additional inactive and/or therapeutic agents that are suitable for treating or preventing, for example, a cancer (e.g., a standard-of-care agent, etc.). The pharmaceutical composition may also comprise one or more carrier, excipient, and/or stabilizer components, and the like. Methods of formulating such pharmaceutical compositions and suitable formulations are known in the art (see, e.g., “Remington's Pharmaceutical Sciences,” Mack Publishing Co., Easton, PA).

Suitable carriers include any material that, when combined with the therapeutic composition, retains the anti-tumor function of the therapeutic composition and is generally non-reactive with the patient's immune system. Pharmaceutically acceptable carriers include any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, mesylate salt, and the like, as well as combinations thereof. In many cases, isotonic agents are included, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the ADC.

A pharmaceutical composition of the present disclosure can be administered by a variety of methods known in the art. The route and/or mode of administration may vary depending upon the desired results. In some embodiments, the therapeutic formulation is solubilized and administered via any route capable of delivering the therapeutic composition to the cancer site. Potentially effective routes of administration include, but are not limited to, parenteral (e.g., intravenous, subcutaneous), intraperitoneal, intramuscular, intratumor, intradermal, intraorgan, orthotopic, and the like. In some embodiments, the administration is intravenous, subcutaneous, intraperitoneal, or intramuscular. The pharmaceutically acceptable carrier should be suitable for the route of administration, e.g., intravenous or subcutaneous administration (e.g., by injection or infusion). Depending on the route of administration, the active compound(s), i.e., the ADC and/or any additional therapeutic agent, may be coated in a material to protect the compound(s) from the action of acids and other natural conditions that may inactivate the compound(s). Administration can be either systemic or local.

The therapeutic compositions disclosed herein may be sterile and stable under the conditions of manufacture and storage, and may be in a variety of forms. These include, for example, liquid, semi-solid, and solid dosage forms, such as liquid solutions (e.g., injectable and infusible solutions), dispersions or suspensions, tablets, pills, powders, liposomes, and suppositories. The form depends on the intended mode of administration and therapeutic application. In some embodiments, the disclosed ADCs can be incorporated into a pharmaceutical composition suitable for parenteral administration. The injectable solution may be composed of either a liquid or lyophilized dosage form in a flint or amber vial, ampule, or pre-filled syringe, or other known delivery or storage device. In some embodiments, one or more of the ADCs or pharmaceutical compositions is supplied as a dry sterilized lyophilized powder or water free concentrate in a hermetically sealed container and can be reconstituted (e.g., with water or saline) to the appropriate concentration for administration to a subject.

Typically, a therapeutically effective amount or efficacious amount of a disclosed composition, e.g., a disclosed ADC, is employed in the pharmaceutical compositions of the present disclosure. The composition, e.g., one comprising an ADC, may be formulated into a pharmaceutically acceptable dosage form by conventional methods known in the art. Dosages and administration protocols for the treatment of cancers using the foregoing methods will vary with the method and the target cancer, and will generally depend on a number of other factors appreciated in the art.

Dosage regimens for compositions disclosed herein, e.g., those comprising ADCs alone or in combination with at least one additional inactive and/or active therapeutic agent, may be adjusted to provide the optimum desired response (e.g., a therapeutic response). For example, a single bolus of one or both agents may be administered at one time, several divided doses may be administered over a predetermined period of time, or the dose of one or both agents may be proportionally increased or decreased as indicated by the exigencies of the therapeutic situation. In some embodiments, treatment involves single bolus or repeated administration of the ADC preparation via an acceptable route of administration. In some embodiments, the ADC is administered to the patient daily, weekly, monthly, or any time period in between. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Dosage values for compositions comprising an ADC and/or any additional therapeutic agent(s), may be selected based on the unique characteristics of the active compound(s), and the particular therapeutic effect to be achieved. A physician or veterinarian can start doses of the ADC employed in the pharmaceutical composition at levels lower than that required to achieve the desired therapeutic effect and gradually increase the dosage until the desired effect is achieved. In general, effective doses of the compositions of the present disclosure, for the treatment of a cancer may vary depending upon many different factors, including means of administration, target site, physiological state of the patient, whether the patient is human or an animal, other medications administered, and whether treatment is prophylactic or therapeutic. The selected dosage level may also depend upon a variety of pharmacokinetic factors including the activity of the particular compositions of the present disclosure employed, or the ester, salt, or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. Treatment dosages may be titrated to optimize safety and efficacy.

Toxicity and therapeutic efficacy of compounds provided herein can be determined by standard pharmaceutical procedures in cell culture or in animal models. For example, LD50, ED50, EC50, and IC50 may be determined, and the dose ratio between toxic and therapeutic effects (LD50/ED50) may be calculated as the therapeutic index. The data obtained from in vitro and in vivo assays can be used in estimating or formulating a range of dosage for use in humans. For example, the compositions and methods disclosed herein may initially be evaluated in xenogeneic cancer models.

In some embodiments, an ADC or composition comprising an ADC is administered on a single occasion. In other embodiments, an ADC or composition comprising an ADC is administered on multiple occasions. Intervals between single dosages can be, e.g., daily, weekly, monthly, or yearly. Intervals can also be irregular, based on measuring blood levels of the administered agent (e.g., the ADC) in the patient in order to maintain a relatively consistent plasma concentration of the agent. The dosage and frequency of administration of an ADC or composition comprising an ADC may also vary depending on whether the treatment is prophylactic or therapeutic. In prophylactic applications, a relatively low dosage may be administered at relatively infrequent intervals over a long period of time. Some patients continue to receive treatment for the rest of their lives. In therapeutic applications, a relatively higher dosage at relatively shorter intervals is sometimes required until progression of the disease is reduced or terminated, and preferably until the patient shows partial or complete amelioration of one or more symptoms of disease. Thereafter, the patient may be administered a lower, e.g., prophylactic regime.

The above therapeutic approaches can be combined with any one of a wide variety of additional surgical, chemotherapy, or radiation therapy regimens. In some embodiments, the ADCs or compositions disclosed herein are co-formulated and/or co-administered with one or more additional therapeutic agents, e.g., one or more chemotherapeutic agents, one or more standard-of-care agents for the particular condition being treated.

Kits for use in the therapeutic and/or diagnostic applications described herein are also provided. Such kits may comprise a carrier, package, or container that is compartmentalized to receive one or more containers such as vials, tubes, and the like, each of the container(s) comprising one of the separate elements to be used in a method disclosed herein. A label may be present on or with the container(s) to indicate that an ADC or composition within the kit is used for a specific therapy or non-therapeutic application, such as a prognostic, prophylactic, diagnostic, or laboratory application. A label may also indicate directions for either in vivo or in vitro use, such as those described herein. Directions and or other information may also be included on an insert(s) or label(s), which is included with or on the kit. The label may be on or associated with the container. A label may be on a container when letters, numbers, or other characters forming the label are molded or etched into the container itself. A label may be associated with a container when it is present within a receptacle or carrier that also holds the container, e.g., as a package insert. The label may indicate that an ADC or composition within the kit is used for diagnosing or treating a condition, such as a cancer a described herein.

In some embodiments, a kit comprises an ADC or composition comprising an ADC. In some embodiments, the kit further comprises one or more additional components, including but not limited to: instructions for use; other reagents, e.g., a therapeutic agent (e.g., a standard-of-care agent); devices, containers, or other materials for preparing the ADC for administration; pharmaceutically acceptable carriers; and devices, containers, or other materials for administering the ADC to a subject. Instructions for use can include guidance for therapeutic applications including suggested dosages and/or modes of administration, e.g., in a patient having or suspected of having a cancer. In some embodiments, the kit comprises an ADC and instructions for use of the ADC in treating, preventing, and/or diagnosing a cancer.

It is known that elevated Bcl-xL expression correlates with resistance to radiation therapy and chemotherapy. Antibody-drug conjugates (ADCs) that may not be sufficiently effective as monotherapy to treat cancer can be administered in combination with other therapeutic agents (including non-targeted and targeted therapeutic agents) or radiation therapy (including radioligand therapy) to provide therapeutic benefit. Without wishing to be bound by theory, it is believed that the ADCs described herein sensitize tumor cells to the treatment with other therapeutic agents (including standard of care chemotherapeutic agents to which the tumor cells may have developed resistance) and/or radiation therapy. In some embodiments, antibody drug conjugates described herein, are administered to a subject having cancer in an amount effective to sensitize the tumor cells. As used herein, the term “sensitize” means that the treatment with ADC increases the potency or efficacy of the treatment with other therapeutic agents and/or radiation therapy against tumor cells.

### Combination Therapies

In some embodiments, the present disclosure provides methods of treatment wherein the antibody-drug conjugates disclosed herein are administered in combination with one or more (e.g., 1 or 2) additional therapeutic agents. Exemplary combination partners are disclosed herein.

In certain embodiments, a combination described herein comprises a PD-1 inhibitor. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is PDR001. PDR001 is also known as Spartalizumab.

In certain embodiments, a combination described herein comprises a LAG-3 inhibitor. In some embodiments, the LAG-3 inhibitor is chosen from LAG525 (Novartis), BMS-986016 (Bristol-Myers Squibb), or TSR-033 (Tesaro).

In certain embodiments, a combination described herein comprises a TIM-3 inhibitor. In some embodiments, the TIM-3 inhibitor is MBG453 (Novartis), TSR-022 (Tesaro), LY-3321367 (Eli Lily), Sym23 (Symphogen), BGB-A425 (Beigene), INCAGN-2390 (Agenus), BMS-986258 (BMS), RO-7121661 (Roche), or LY-3415244 (Eli Lilly).

In certain embodiments, a combination described herein comprises a PDL1 inhibitor. In one embodiment, the PDL1 inhibitor is chosen from FAZ053 (Novartis), atezolizumab (Genentech), durvalumab (Astra Zeneca), or avelumab (Pfizer).

In certain embodiments, a combination described herein comprises a GITR agonist. In some embodiments, the GITR agonist is chosen from GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

In some embodiments, a combination described herein comprises an IAP inhibitor. In some embodiments, the IAP inhibitor comprises LCL161 or a compound disclosed in International Application Publication No. WO 2008/016893.

In an embodiment, the combination comprises an mTOR inhibitor, e.g., RAD001 (also known as everolimus).

In an embodiment, the combination comprises a HDAC inhibitor, e.g., LBH589. LBH589 is also known as panobinostat.

In an embodiment, the combination comprises an IL-17 inhibitor, e.g., CJM112.

In certain embodiments, a combination described herein comprises an estrogen receptor (ER) antagonist. In some embodiments, the estrogen receptor antagonist is used in combination with a PD-1 inhibitor, a CDK4/6 inhibitor, or both. In some embodiments, the combination is used to treat an ER positive (ER+) cancer or a breast cancer (e.g., an ER+ breast cancer).

In some embodiments, the estrogen receptor antagonist is a selective estrogen receptor degrader (SERD). SERDs are estrogen receptor antagonists which bind to the receptor and result in e.g., degradation or down-regulation of the receptor (Boer K. et al., (2017) Therapeutic Advances in Medical Oncology 9 (7):465-479). ER is a hormone-activated transcription factor important for e.g., the growth, development and physiology of the human reproductive system. ER is activated by, e.g., the hormone estrogen (17beta estradiol). ER expression and signaling is implicated in cancers (e.g., breast cancer), e.g., ER positive (ER+) breast cancer. In some embodiments, the SERD is chosen from LSZ102, fulvestrant, brilanestrant, or elacestrant.

In some embodiments, the SERD comprises a compound disclosed in International Application Publication No. WO 2014/130310, which is hereby incorporated by reference in its entirety.

In some embodiments, the SERD comprises LSZ102. LSZ102 has the chemical name: (E)-3-(4-((2-(2-(1,1-difluoroethyl)-4-fluorophenyl)-6-hydroxybenzo[b]thiophen-3-yl)oxy)phenyl)acrylic acid. In some embodiments, the SERD comprises fulvestrant (CAS Registry Number: 129453-61-8), or a compound disclosed in International Application Publication No. WO 2001/051056, which is hereby incorporated by reference in its entirety. In some embodiments, the SERD comprises elacestrant (CAS Registry Number: 722533-56-4), or a compound disclosed in U.S. Pat. No. 7,612,114, which is incorporated by reference in its entirety. Elacestrant is also known as RAD1901, ER-306323 or (6R)-6-{2-[Ethyl ({4-[2-(ethylamino)ethyl]phenyl}methyl)amino]-4-methoxyphenyl}-5,6,7,8-tetrahydronaphthalen-2-ol. Elacestrant is an orally bioavailable, non-steroidal combined selective estrogens receptor modulator (SERM) and a SERD. Elacestrant is also disclosed, e.g., in Garner F et al., (2015) Anticancer Drugs 26 (9):948-56. In some embodiments, the SERD is brilanestrant (CAS Registry Number: 1365888-06-7), or a compound disclosed in International Application Publication No. WO 2015/136017, which is incorporated by reference in its entirety.

In some embodiments, the SERD is chosen from RU 58668, GW7604, AZD9496, bazedoxifene, pipendoxifene, arzoxifene, OP-1074, or acolbifene, e.g., as disclosed in McDonell et al. (2015) Journal of Medicinal Chemistry 58 (12)4883-4887.

Other exemplary estrogen receptor antagonists are disclosed, e.g., in WO 2011/156518, WO 2011/159769, WO 2012/037410, WO 2012/037411, and US 2012/0071535, all of which are hereby incorporated by reference in their entirety

In certain embodiments, a combination described herein comprises an inhibitor of Cyclin-Dependent Kinases 4 or 6 (CDK4/6). In some embodiments, the CDK4/6 inhibitor is used in combination with a PD-1 inhibitor, an estrogen receptor (ER) antagonist, or both. In some embodiments, the combination is used to treat an ER positive (ER+) cancer or a breast cancer (e.g., an ER+ breast cancer). In some embodiments, the CDK4/6 inhibitor is chosen from ribociclib, abemaciclib (Eli Lilly), or palbociclib.

In some embodiments, the CDK4/6 inhibitor comprises ribociclib (CAS Registry Number: 1211441-98-3), or a compound disclosed in U.S. Pat. Nos. 8,415,355 and 8,685,980, which are incorporated by reference in their entirety.

In some embodiments, the CDK4/6 inhibitor comprises a compound disclosed in International Application Publication No. WO 2010/020675 and U.S. Pat. Nos. 8,415,355 and 8,685,980, which are incorporated by reference in their entirety.

In some embodiments, the CDK4/6 inhibitor comprises ribociclib (CAS Registry Number: 1211441-98-3). Ribociclib is also known as LEE011, KISQALI®, or 7-cyclopentyl-N,N-dimethyl-2-((5-(piperazin-1-yl)pyridin-2-yl)amino)-7H-pyrrolo[2,3-d]pyrimidine-6-carboxamide.

In some embodiments, the CDK4/6 inhibitor comprises abemaciclib (CAS Registry Number: 1231929-97-7). Abemaciclib is also known as LY835219 or N-[5-[(4-Ethyl-1-piperazinyl)methyl]-2-pyridinyl]-5-fluoro-4-[4-fluoro-2-methyl-1-(1-methylethyl)-1H-benzimidazol-6-yl]-2-pyrimidinamine. Abemaciclib is a CDK inhibitor selective for CDK4 and CDK6 and is disclosed, e.g., in Torres-Guzman R et al. (2017) Oncotarget 10.18632/oncotarget.17778.

In some embodiments, the CDK4/6 inhibitor comprises palbociclib (CAS Registry Number: 571190-30-2). Palbociclib is also known as PD-0332991, IBRANCE® or 6-Acetyl-8-cyclopentyl-5-methyl-2-{[5-(1-piperazinyl)-2-pyridinyl]amino}pyrido[2,3-d]pyrimidin-7(8H)-one. Palbociclib inhibits CDK4 with an IC50 of 11 nM, and inhibits CDK6 with an IC50 of 16 nM, and is disclosed, e.g., in Finn et al. (2009) Breast Cancer Research 11(5):R77.

In certain embodiments, a combination described herein comprises an inhibitor of chemokine (C—X—C motif) receptor 2 (CXCR2). In some embodiments, the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide, danirixin, reparixin, or navarixin.

In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK)(e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110. In other embodiments, the CSF-1/1R binding agent is pexidartinib.

In certain embodiments, a combination described herein comprises a c-MET inhibitor. c-MET, a receptor tyrosine kinase overexpressed or mutated in many tumor cell types, plays key roles in tumor cell proliferation, survival, invasion, metastasis, and tumor angiogenesis. Inhibition of c-MET may induce cell death in tumor cells overexpressing c-MET protein or expressing constitutively activated c-MET protein. In some embodiments, the c-MET inhibitor is chosen from capmatinib (INC280), JNJ-3887605, AMG 337, LY2801653, MSC2156119J, crizotinib, tivantinib, or golvatinib.

In certain embodiments, a combination described herein comprises a transforming growth factor beta (also known as TGF-β TGFβ, TGFb, or TGF-beta, used interchangeably herein) inhibitor. In some embodiments, the TGF-β inhibitor is chosen from fresolimumab or XOMA 089.

In certain embodiments, a combination described herein comprises an adenosine A2a receptor (A2aR) antagonist (e.g., an inhibitor of A2aR pathway, e.g., an adenosine inhibitor, e.g., an inhibitor of A2aR or CD-73). In some embodiments, the A2aR antagonist is used in combination with a PD-1 inhibitor, and one or more (e.g., two, three, four, five, or all) of a CXCR2 inhibitor, a CSF-1/1R binding agent, LAG-3 inhibitor, a GITR agonist, a c-MET inhibitor, or an IDO inhibitor. In some embodiments, the combination is used to treat a pancreatic cancer, a colorectal cancer, a gastric cancer, or a melanoma (e.g., a refractory melanoma). In some embodiments, the A2aR antagonist is chosen from PBF509 (NIR178)(Palobiofarma/Novartis), CPI444/V81444 (Corvus/Genentech), AZD4635/HTL-1071 (AstraZeneca/Heptares), Vipadenant (Redox/Juno), GBV-2034 (Globavir), AB928 (Arcus Biosciences), Theophylline, Istradefylline (Kyowa Hakko Kogyo), Tozadenant/SYN-115 (Acorda), KW-6356 (Kyowa Hakko Kogyo), ST-4206 (Leadiant Biosciences), or Preladenant/SCH 420814 (Merck/Schering). Without wishing to be bound by theory, it is believed that in some embodiments, inhibition of A2aR leads to upregulation of IL-1b.

In certain embodiments, a combination described herein comprises an inhibitor of indoleamine 2,3-dioxygenase (IDO) and/or tryptophan 2,3-dioxygenase (TDO). In some embodiments, the IDO inhibitor is used in combination with a PD-1 inhibitor, and one or more (e.g., two, three, four, or all) of a TGF-β inhibitor, an A2aR antagonist, a CSF-1/1R binding agent, a c-MET inhibitor, or a GITR agonist. In some embodiments, the combination is used to treat a pancreatic cancer, a colorectal cancer, a gastric cancer, or a melanoma (e.g., a refractory melanoma). In some embodiments, the IDO inhibitor is chosen from (4E)-4-[(3-chloro-4-fluoroanilino)-nitrosomethylidene]-1,2,5-oxadiazol-3-amine (also known as epacadostat or INCB24360), indoximod (NLG8189), (1-methyl-D-tryptophan), α-cyclohexyl-5H-Imidazo[5,1-a]isoindole-5-ethanol (also known as NLG919), indoximod, BMS-986205 (formerly F001287).

In certain embodiments, a combination described herein comprises a Galectin, e.g., Galectin-1 or Galectin-3, inhibitor. In some embodiments, the combination comprises a Galectin-1 inhibitor and a Galectin-3 inhibitor. In some embodiments, the combination comprises a bispecific inhibitor (e.g., a bispecific antibody molecule) targeting both Galectin-1 and Galectin-3. In some embodiments, the Galectin inhibitor is used in combination with one or more therapeutic agents described herein. In some embodiments, the Galectin inhibitor is chosen from an anti-Galectin antibody molecule, GR-MD-02 (Galectin Therapeutics), Galectin-3C (Mandal Med), Anginex, or OTX-008 (OncoEthix, Merck).

In some embodiments, a combination described herein comprises an inhibitor of the MAP kinase pathway including ERK inhibitors, MEK inhibitors and RAF inhibitors.

In some embodiments, a combination described herein comprises a MEK inhibitor. In some embodiments, the MEK inhibitor is chosen from Trametinib, selumetinib, AS703026, BIX 02189, BIX 02188, CI-1040, PD0325901, PD98059, U0126, XL-518, G-38963, or G02443714.

In some embodiments, the MEK inhibitor is trametinib. Trametinib is also known as JTP-74057, TMT212, CFF272, N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1 (2H)-yl}phenyl)acetamide, or Mekinist (CAS Number 871700-17-3).

In some embodiments, the MEK inhibitor comprises selumetinib which has the chemical name: (5-[(4-bromo-2-chlorophenyl)amino]-4-fluoro-N-(2-hydroxyethoxy)-1-methyl-1H-benzimidazole-6-carboxamide. Selumetinib is also known as AZD6244 or ARRY 142886, e.g., as described in PCT Publication No. WO2003077914.

In some embodiments, the MEK inhibitor comprises AS703026, BIX 02189 or BIX 02188.

In some embodiments, the MEK inhibitor comprises 2-[(2-Chloro-4-iodophenyl)amino]-N-(cyclopropylmethoxy)-3,4-difluoro-benzamide (also known as CI-1040 or PD184352), e.g., as described in PCT Publication No. WO2000035436).

In some embodiments, the MEK inhibitor comprises N-[(2R)-2,3-Dihydroxypropoxy]-3,4-difluoro-2-[(2-fluoro-4-iodophenyl)amino]-benzamide (also known as PD0325901), e.g., as described in PCT Publication No. WO2002006213).

In some embodiments, the MEK inhibitor comprises 2′-amino-3′-methoxyflavone (also known as PD98059) which is available from Biaffin GmbH & Co., KG, Germany.

In some embodiments, the MEK inhibitor comprises 2,3-bis[amino[(2-aminophenyl)thio]methylene]-butanedinitrile (also known as U0126), e.g., as described in U.S. Pat. No. 2,779,780).

In some embodiments, the MEK inhibitor comprises XL-518 (also known as GDC-0973) which has a CAS No. 1029872-29-4 and is available from ACC Corp.

In some embodiments, the MEK inhibitor comprises G-38963.

In some embodiments, the MEK inhibitor comprises G02443714 (also known as AS703206)

Additional examples of MEK inhibitors are disclosed in WO 2013/019906, WO 03/077914, WO 2005/121142, WO 2007/04415, WO 2008/024725 and WO 2009/085983, the contents of which are incorporated herein by reference. Further examples of MEK inhibitors include, but are not limited to, 2,3-Bis[amino[(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione](also known as E6201, described in PCT Publication No. WO2003076424); vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

In some embodiments, a combination described herein comprises a RAF inhibitor.

RAF inhibitors include, but are not limited to, Vemurafenib (or Zelboraf®, PLX-4032, CAS 918504-65-1), GDC-0879, PLX-4720 (available from Symansis), Dabrafenib (or GSK2118436), LGX 818, CEP-32496, UI-152, RAF 265, Regorafenib (BAY 73-4506), CCT239065, or Sorafenib (or Sorafenib Tosylate, or Nexavar®).

In some embodiments, the RAF inhibitor is Dabrafenib.

In some embodiments, the RAF inhibitor is LXH254.

In some embodiments, a combination described herein comprises an ERK inhibitor.

ERK inhibitors include, but are not limited to, LTT462, ulixertinib (BVD-523), LY3214996, GDC-0994, KO-947 and MK-8353.

In some embodiments, the ERK inhibitor is LTT462. LTT462 is 4-(3-amino-6-((1S,3S,4S)-3-fluoro-4-hydroxy-cyclohexyl) pyrazin-2-yl)-N—((S)-1-(3-bromo-5-fluorophenyl)-2-(methylamino)-ethyl)-2-fluorobenzamide and is the compound of the following structure:

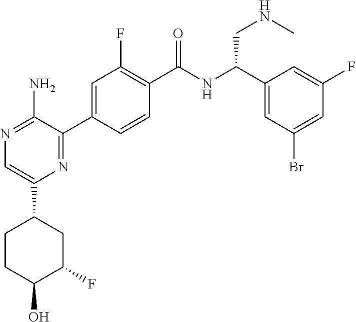 [SMILES: CNC[C@@H](NC(=O)c1ccc(-c2nc([C@H]3CC[C@H](O)[C@@H](F)C3)cnc2N)cc1F)c1cc(F)cc(Br)c1]

The preparation of LTT462 is described in PCT patent application publication WO2015/066188. LTT462 is an inhibitor of extracellular signal-regulated kinases 1 and 2 (ERK 1/2).

In some embodiments, a combination described herein comprises a taxane, a vinca alkaloid, a MEK inhibitor, an ERK inhibitor, or a RAF inhibitor.

In some embodiments, a combination described herein comprises at least two inhibitors selected, independently, from a MEK inhibitor, an ERK inhibitor, and a RAF inhibitor.

In some embodiments, a combination described herein comprises an anti-mitotic drug.

In some embodiments, a combination described herein comprises a taxane.

Taxanes include, but are not limited to, docetaxel, paclitaxel, or cabazitaxel. In some embodiments, the taxane is docetaxel.

In some embodiments, a combination described herein comprises a vinca alkaloid.

Vinca alkaloids include, but are not limited to, vincristine, vinblastine, and leurosine.

In some embodiments, a combination described herein comprises a topoisomerase inhibitor.

Topoisomerase inhibitors include, but are not limited to, topotecan, irinotecan, camptothecin, diflomotecan, lamellarin D, ellipticines, etoposide (VP-16), teniposide, doxorubicin, daunorubicin, mitoxantrone, amsacrine, aurintricarboxylic acid, and HU-331.

In one embodiment, a combination described herein includes an interleukin-1 beta (IL-1β) inhibitor. In some embodiments, the IL-1β inhibitor is chosen from canakinumab, gevokizumab, Anakinra, or Rilonacept.

In certain embodiments, a combination described herein comprises an IL-15/IL-15Ra complex. In some embodiments, the IL-15/IL-15Ra complex is chosen from NIZ985 (Novartis), ATL-803 (Altor) or CYP0150 (Cytune).

In certain embodiments, a combination described herein comprises a mouse double minute 2 homolog (MDM2) inhibitor. The human homolog of MDM2 is also known as HDM2. In some embodiments, an MDM2 inhibitor described herein is also known as a HDM2 inhibitor. In some embodiments, the MDM2 inhibitor is chosen from HDM201 or CGM097.

In an embodiment the MDM2 inhibitor comprises (S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one (also known as CGM097) or a compound disclosed in PCT Publication No. WO 2011/076786 to treat a disorder, e.g., a disorder described herein). In one embodiment, a therapeutic agent disclosed herein is used in combination with CGM097.

In some embodiments, a combination described herein comprises a hypomethylating agent (HMA). In some embodiments, the HMA is chosen from decitabine or azacitidine.

In some embodiments, a combination described herein comprises a glucocorticoid. In some embodiments, the glucocorticoid is dexamethasone.

In some embodiments, a combination described herein comprises a nucleoside analog. In some embodiments, the nucleoside analog is gemcitabine.

In some embodiments, a combination described herein comprises asparaginase.

In certain embodiments, a combination described herein comprises an inhibitor acting on any pro-survival proteins of the Bcl2 family. In certain embodiments, a combination described herein comprises a Bcl-2 inhibitor. In some embodiments, the Bcl-2 inhibitor is venetoclax (also known as ABT-199):

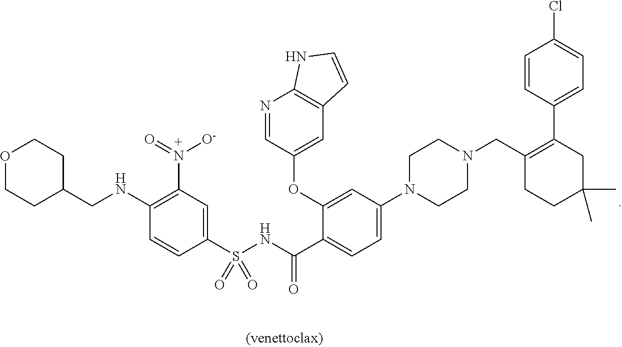

In one embodiment, the Bcl-2 inhibitor is selected from the compounds described in WO 2013/110890 and WO 2015/011400. In some embodiments, the Bcl-2 inhibitor comprises navitoclax (ABT-263), ABT-737, BP1002, SPC2996, APG-1252, obatoclax mesylate (GX15-070 MS), PNT2258, Zn-d5, BGB-11417, or oblimersen (G3139). In some embodiments, the Bcl-2 inhibitor is N-(4-hydroxyphenyl)-3-[6-[(3S)-3-(morpholinomethyl)-3,4-dihydro-1H-isoquinoline-2-carbonyl]-1,3-benzodioxol-5-yl]-N-phenyl-5,6,7,8-tetrahydroindolizine-1-carboxamide, compound A1:

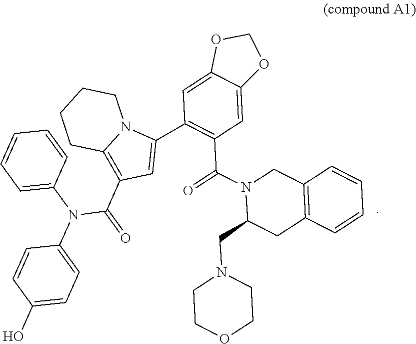 [SMILES: O=C(c1cc(-c2cc3c(cc2C(=O)N2Cc4ccccc4C[C@H]2CN2CCOCC2)OCO3)n2c1CCCC2)N(c1ccccc1)c1ccc(O)cc1]

In some embodiments, the Bcl-2 inhibitor is(S)-5-(5-chloro-2-(3-(morpholinomethyl)-1,2,3,4-tetrahydroisoquinoline-2-carbonyl)phenyl)-N-(5-cyano-1,2-dimethyl-1H-pyrrol-3-yl)-N-(4-hydroxyphenyl)-1,2-dimethyl-1H-pyrrole-3-carboxamide), compound A2:

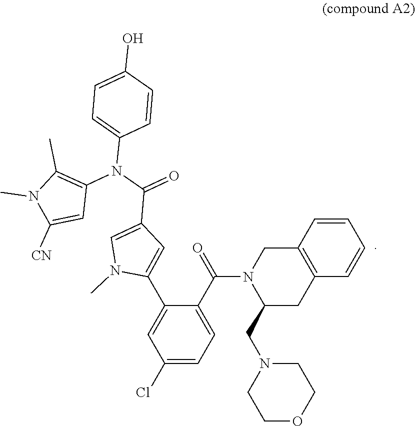 [SMILES: Cc1c(N(C(=O)c2cc(-c3cc(Cl)ccc3C(=O)N3Cc4ccccc4C[C@H]3CN3CCOCC3)n(C)c2)c2ccc(O)cc2)cc(C#N)n1C]

In one embodiment, the antibody-drug conjugates or combinations disclosed herein are suitable for the treatment of cancer in vivo. For example, the combination can be used to inhibit the growth of cancerous tumors. The combination can also be used in combination with one or more of: a standard of care treatment (e.g., for cancers or infectious disorders), a vaccine (e.g., a therapeutic cancer vaccine), a cell therapy, a hormone therapy (e.g., with anti-estrogens or anti-androgens), a radiation therapy, surgery, or any other therapeutic agent or modality, to treat a disorder herein. For example, to achieve antigen-specific enhancement of immunity, the combination can be administered together with an antigen of interest. A combination disclosed herein can be administered in either order or simultaneously.

### ADDITIONAL EMBODIMENTS

The disclosure provides the following additional embodiments for linker-drug groups, antibody-drug conjugates, linker groups, and methods of conjugation.

### Linker-Drug Group

In some embodiments, the Linker-Drug group of the invention may be a compound having the structure of Formula (A′), or a pharmaceutically acceptable salt thereof:

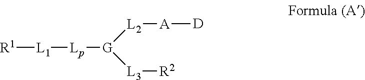

wherein:
   R¹ is a reactive group;
   L₁ is a bridging spacer;
   Lp is a bivalent peptide spacer;
   G-L₂-A is a self-immolative spacer;
   R² is a hydrophilic moiety;
   L₂ is a bond, a methylene, a neopentylene or a C₂-C₃alkenylene;
   A is a bond, —OC(═O)—*,

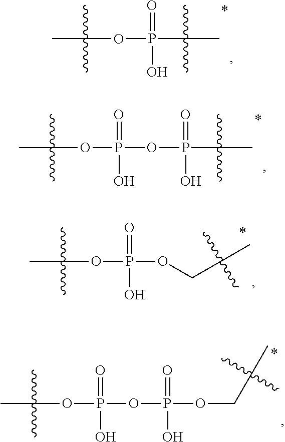 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety; and
D is a Drug moiety that is capable of inhibiting the activity of the Bcl-xL protein when, e.g., released from the Antibody Drug Conjugates or immunoconjugates disclosed herein.

Certain aspects and examples of the Linker-Drug group of the invention are provided in the following listing of enumerated embodiments. It will be recognized that features specified in each embodiment may be combined with other specified features to provide further embodiments of the present invention.

Embodiment 1. The compound of Formula (A′), or pharmaceutically acceptable salt thereof, wherein:
R¹ is a reactive group;
L₁ is a bridging spacer;
Lp is a bivalent peptide spacer comprising two to four amino acid residues;
G-L₂-A is a self-immolative spacer;
R² is a hydrophilic moiety;
L₂ is a bond, a methylene, a neopentylene or a C₂-C₃alkenylene;
A is a bond, —OC(═O)—*,

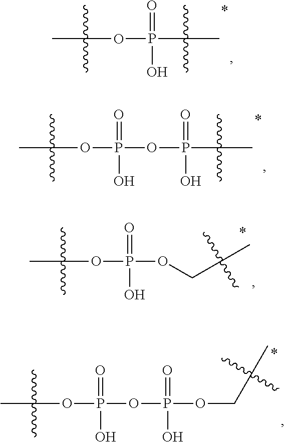 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety; and
D is a Drug moiety as defined herein, e.g., a Bcl-xL inhibitor.
   Embodiment 2. The compound of Formula (A′), or pharmaceutically acceptable salt thereof, wherein:
R¹ is a reactive group;
L₁ is a bridging spacer;
Lp is a bivalent peptide spacer comprising two to four amino acid residues;
the

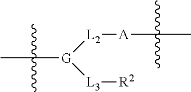

group is selected from:

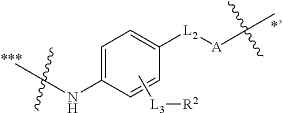

wherein the * of

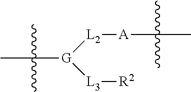

indicates the point of attachment to D (e.g., to an N or a O of the Drug moiety), the *** of

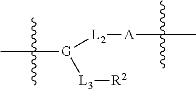

indicates the point of attachment to Lp;
R² is a hydrophilic moiety;
L₂ is a bond, a methylene, a neopentylene or a C₂-C₃alkenylene;
A is a bond, —OC(═O)—*,

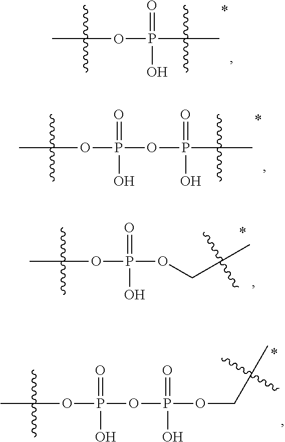 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety; and
D is a Drug moiety as defined herein, e.g., a Bcl-xL inhibitor.
   Embodiment 3. The compound of Formula (A′), or pharmaceutically acceptable salt thereof, having the structure of Formula (B′):

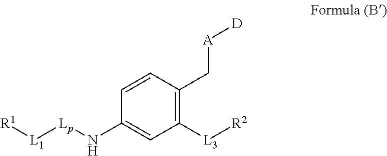

wherein:
   R¹ is a reactive group;
   L₁ is a bridging spacer;
   Lp is a bivalent peptide spacer comprising two to four amino acid residues;
   R² is a hydrophilic moiety;
   A is a bond, —OC(═O)—*,

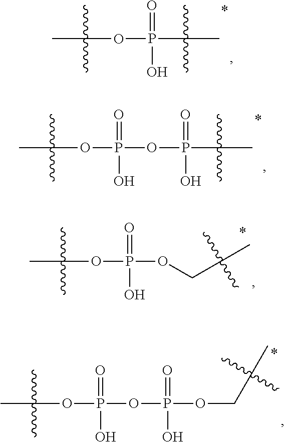 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety; and
D is a Drug moiety as defined herein and comprising an N, wherein D is connected to A via a direct bond from A to the N of the Drug moiety.
   Embodiment 4. The compound of Formula (A′) or of any one of Embodiments 1 to 3, or pharmaceutically acceptable salt thereof, wherein:
R¹ is

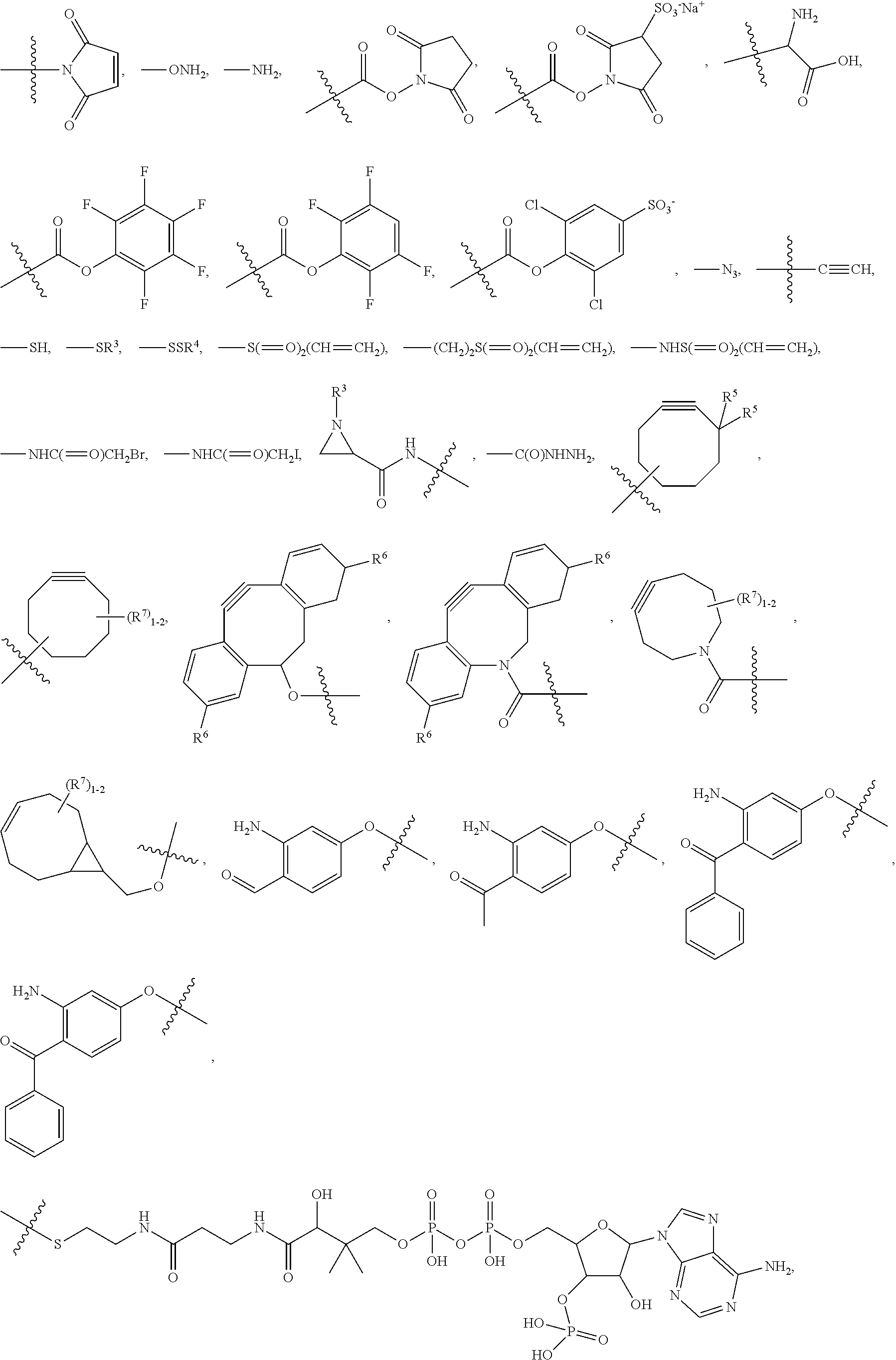

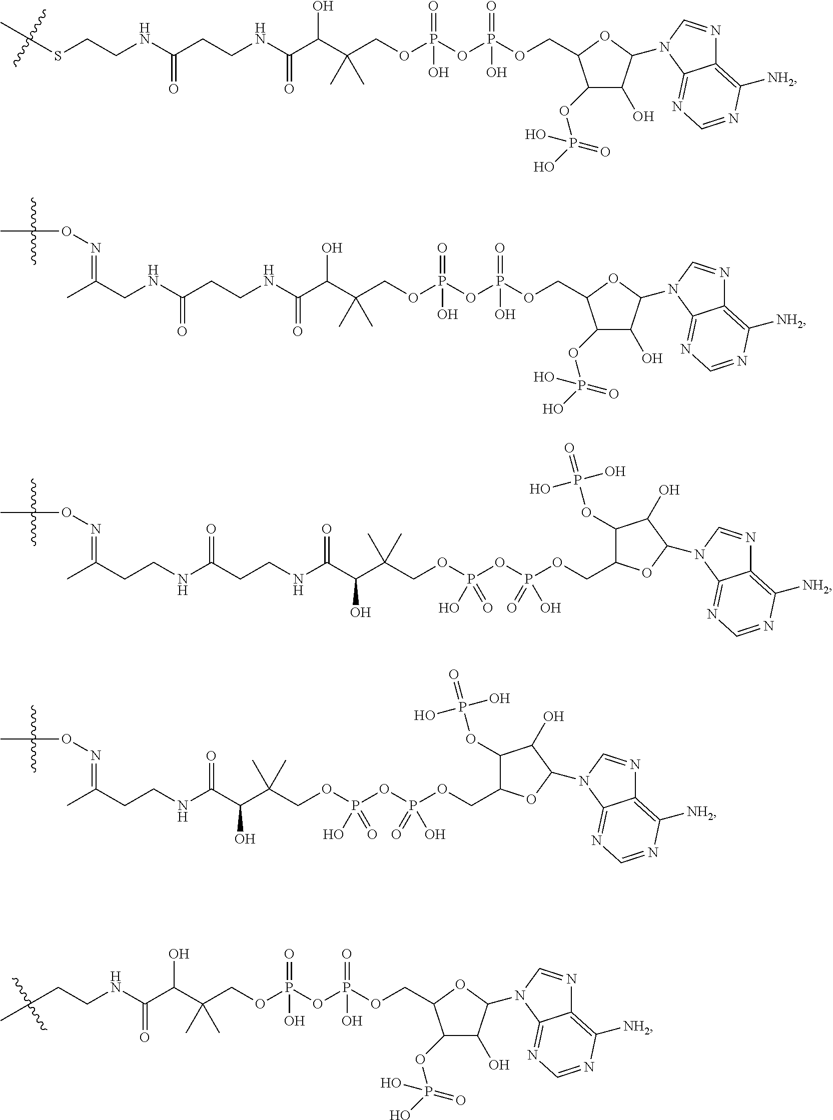

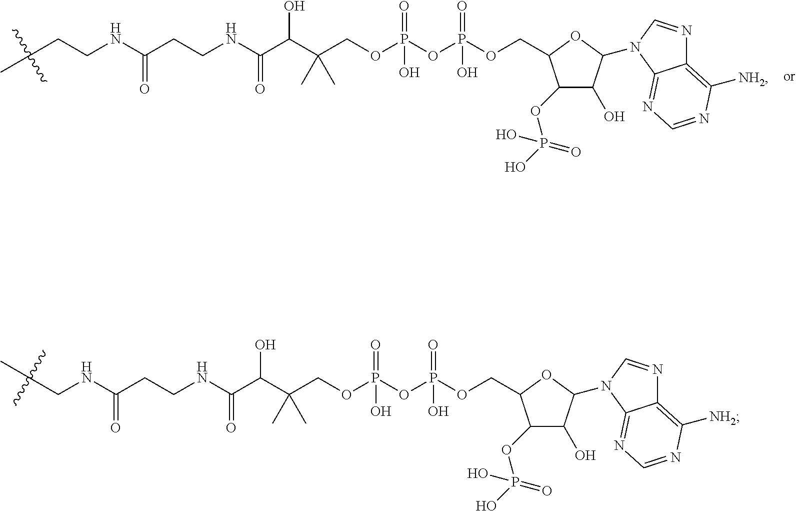 [SMILES: *CCNC(=O)CCNC(=O)C(O)C(C)(C)COP(=O)(O)OP(=O)(O)OCC1OC(n2cnc3c(N)ncnc32)C(O)C1OP(=O)(O)O ; *CNC(=O)CCNC(=O)C(O)C(C)(C)COP(=O)(O)OP(=O)(O)OCC1OC(n2cnc3c(N)ncnc32)C(O)C1OP(=O)(O)O]

L₁ is *—C(═O)(CH₂)ₘO(CH₂)ₘ—**; —C(═O)((CH₂)ₘO)ₜ(CH₂)ₙ—**; —C(═O)(CH₂)ₘ—**; —C(═O)NH(CH₂)ₘO)ₜ(CH₂)ₙ—*; —C(═O)O(CH₂)ₘSSC(R₃)₂(CH₂)ₘC(═O)NR₃(CH₂)ₘNR₃C(═O)(CH₂)ₘ—**; —C(═O)O(CH₂)ₘC(═O)NH(CH₂)ₘ—**; —C(═O)(CH₂)ₘNH(CH₂)ₘ—**; *—C(═O)(CH₂)ₘNH(CH₂)ₙC(═O)—**; —C(═O)(CH₂)ₘX₁(CH₂)ₘ—*; *—C(═O)(CH₂)ₘO)ₜ(CH₂)ₙX₁(CH₂)ₙ—*; —C(═O)(CH₂)ₘNHC(═O)(CH₂)ₙ—**; —C(═O)((CH₂)ₘO)ₜ(CH₂)ₙNHC(═O)(CH₂)ₙ—**; —C(═O)(CH₂)ₘNHC(═O)(CH₂)ₙX₁(CH₂)ₙ—**; —C(═O)((CH₂)ₘO)ₜ(CH₂)ₙNHC(═O)(CH₂)ₙX₁(CH₂)ₙ—**, —C(═O)((CH₂)ₘO)ₜ(CH₂)ₙC(═O)NH(CH₂)ₘ—**; —C(═O)(CH₂)ₘC(R₃)₂—**; or —C(═O)(CH₂)ₘC(═O)NH(CH₂)ₘ—**,
where the * of L₁ indicates the point of attachment to Lp, and the ** of L₁ indicates the point of attachment to R¹;
R² is a hydrophilic moiety selected from polyethylene glycol, polyalkylene glycol, a sugar, an oligosaccharide, a polypeptide or C₂-C₆alkyl substituted with 1 to 3

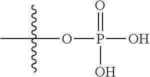 [SMILES: *OP(=O)(O)O]

groups;
each R³ is independently selected from H and C₁-C₆alkyl;
R₄ is 2-pyridyl or 4-pyridyl;
each R⁵ is independently selected from H, C₁-C₆alkyl, F, Cl, and —OH;
each R₆ is independently selected from H, C₁-C₆alkyl, F, Cl, —NH₂, —OCH₃, —OCH₂CH₃, —N(CH₃)₂, —CN, —NO₂ and —OH;
each R₇ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
X₁ is

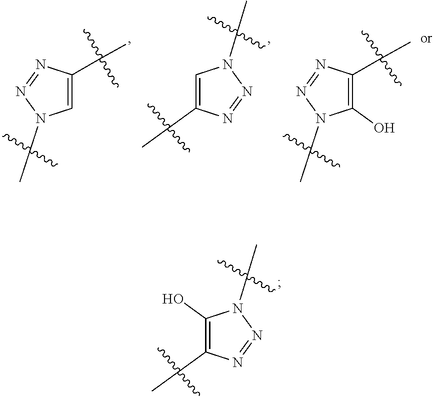

each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15,
16, 17, 18, 19, 20, 21, 22, 23, 24, 25, 26, 27, 28, 29 and 30; Lp is a bivalent peptide spacer comprising an amino acid residue selected from glycine, valine, citrulline, lysine, isoleucine, phenylalanine, methionine, asparagine, proline, alanine, leucine, tryptophan, and tyrosine;
A is a bond, —OC(═O)—*,

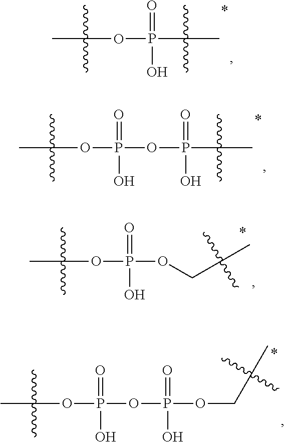 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety having the structure

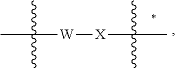

where
   W is —CH₂O—**, —CH₂N(R^{b})C(═O)O—**, —NHC(═O)C(R^{b})₂NHC(═O)O—**, —NHC(═O)C(R^{b})₂NH—**, —NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²; and
   the * of L₃ indicates the point of attachment to R²; and
D is a Drug moiety as defined herein and comprising an N or an O, wherein D is connected to A via a direct bond from A to the N or the O of the Drug moiety.
   Embodiment 5. The compound of Formula (A′) or of any one of Embodiments 1 to 4, or pharmaceutically acceptable salt thereof, wherein:
R¹ is

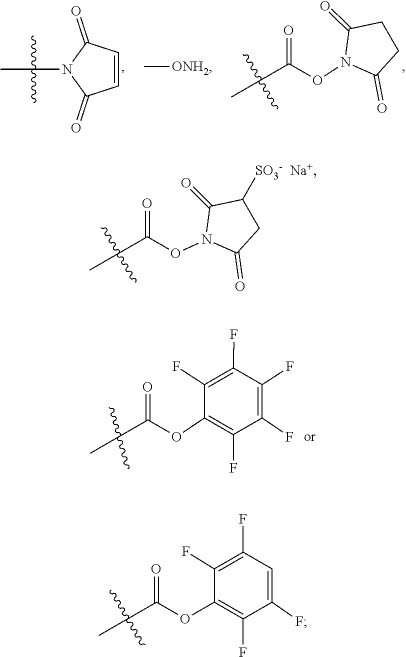

L₁ is *—C(═O)(CH₂)ₘO(CH₂)ₘ—**; *—C(═O)(CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

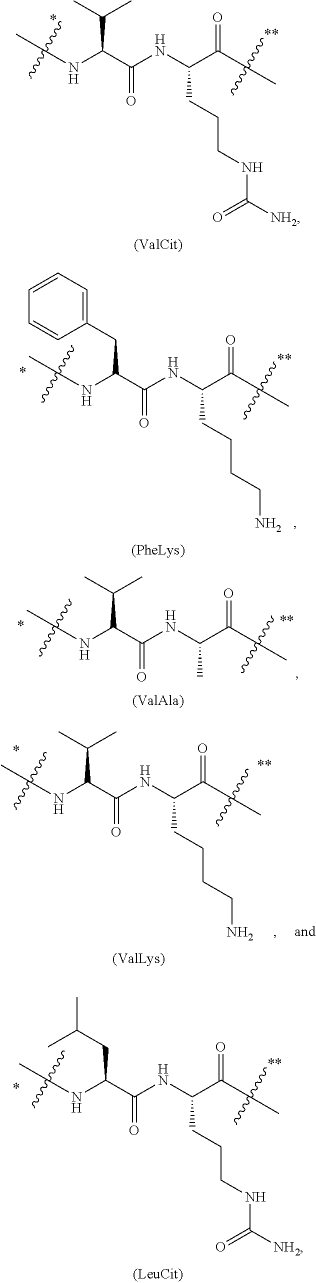

where the * of Lp indicates the attachment point to L₁ and the ** of Lp indicates the attachment point to the —NH— group of G;
L₃ is a spacer moiety having the structure

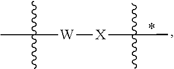

where
   W is —CH₂O—**, —CH₂N(R^{b})C(═O)O—**, —NHC(═O)CH₂NHC(═O)O—**, —NHC(═O)CH₂NH—**, —NHC(═O)CH₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

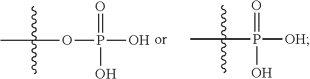 [SMILES: *OP(=O)(O)O ; *P(=O)(O)O]

A is a bond, —OC(═O)—*,

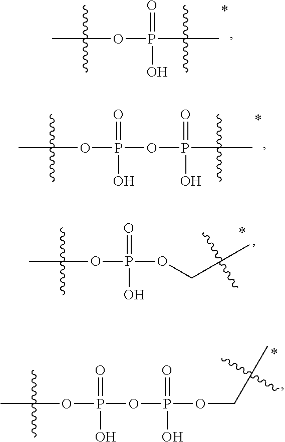 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Drug moiety as defined herein and comprising an N or an O, wherein D is connected to A via a direct bond from A to the N or the O of the Drug moiety.
   Embodiment 6. The compound of Formula (A′) or of any one of Embodiments 1 to 5, or pharmaceutically acceptable salt thereof, wherein:
R¹ is

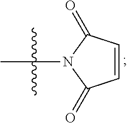 [SMILES: *N1C(=O)C=CC1=O]

L₁ is *—C(═O)(CH₂)ₘO(CH₂)ₘ—*; *—C(═O)(CH₂)ₘO)ₜ(CH₂)ₙ—**; *—C(═O)(CH₂)ₘ—*; or *—C(═O)NH((CH₂)ₘO)ₜ(CH₂)ₙ—, where the * of L₁ indicates the point of attachment to Lp, and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

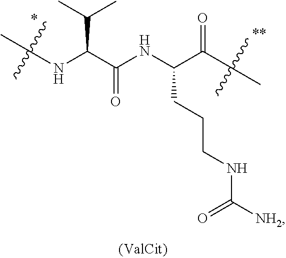 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

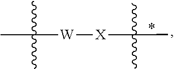

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

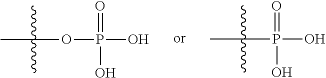 [SMILES: *OP(=O)(O)O ; *P(=O)(O)O]

groups;
A is a bond, —OC(═O)—*,

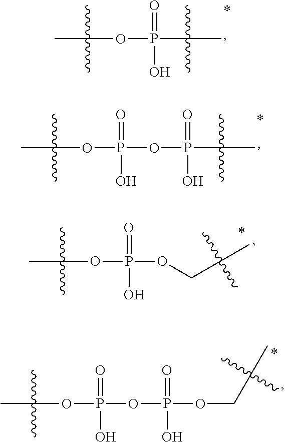 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
and
D is a Drug moiety as defined herein and comprising an N or an O, wherein D is connected to A via a direct bond from A to the N or the O of the Drug moiety.
   Embodiment 7. The compound of Formula (A′) or of any one of Embodiments 1 to 6, or pharmaceutically acceptable salt thereof, wherein:
R¹ is

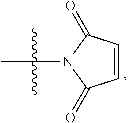 [SMILES: *N1C(=O)C=CC1=O]

L₁ is *—C(═O)(CH₂)ₘO(CH₂)ₘ—*; *—C(═O)(CH₂)ₘO)ₜ(CH₂)ₙ—*; *—C(═O)(CH₂)ₘ—*; or *—C(═O)NH((CH₂)ₘO)ₜ(CH₂)ₙ—, where the * of L₁ indicates the point of attachment to Lp and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

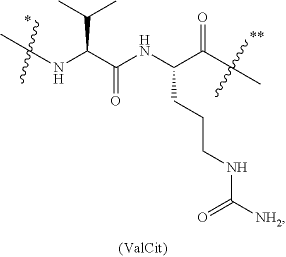 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

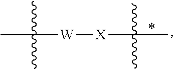

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R_{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

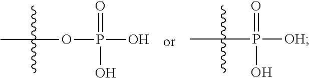 [SMILES: *OP(=O)(O)O ; *P(=O)(O)O]

A is a bond or —OC(═O) *, in which * indicates the attachment point to D;
and
D is a Drug moiety as defined herein and comprising an N or an O, wherein D is connected to A via a direct bond from A to the N or the O of the Drug moiety.
   Embodiment 8. The compound of Formula (A′) or of any one of Embodiments 1 to 7, or pharmaceutically acceptable salt thereof, wherein:
R¹ is

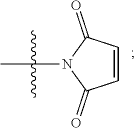 [SMILES: *N1C(=O)C=CC1=O]

L₁ is *—C(═O)(CH₂)ₘO(CH₂)ₘ—**; *—C(═O)(CH₂)ₘO)ₜ(CH₂)ₙ**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

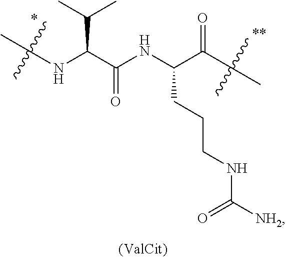 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

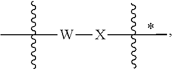

where
   W is —CH₂O—**, —CH₂N(R^{b})C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

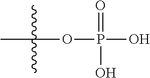 [SMILES: *OP(=O)(O)O]

groups;
A is a bond or —OC(═O)* in which * indicates the attachment point to D;
and
D is a Drug moiety as defined herein and comprising an N or an O, wherein D is connected to A via a direct bond from A to the N or the O of the Drug moiety.
   Embodiment 9. The compound of Formula (A′) or of any one of Embodiments 1 to 8, or pharmaceutically acceptable salt thereof, wherein R¹ is a reactive group selected from Table 3.
   Embodiment 10. The compound of Formula (A′) or of any one of Embodiments 1 to 9, or pharmaceutically acceptable salt thereof, wherein:
R¹ is

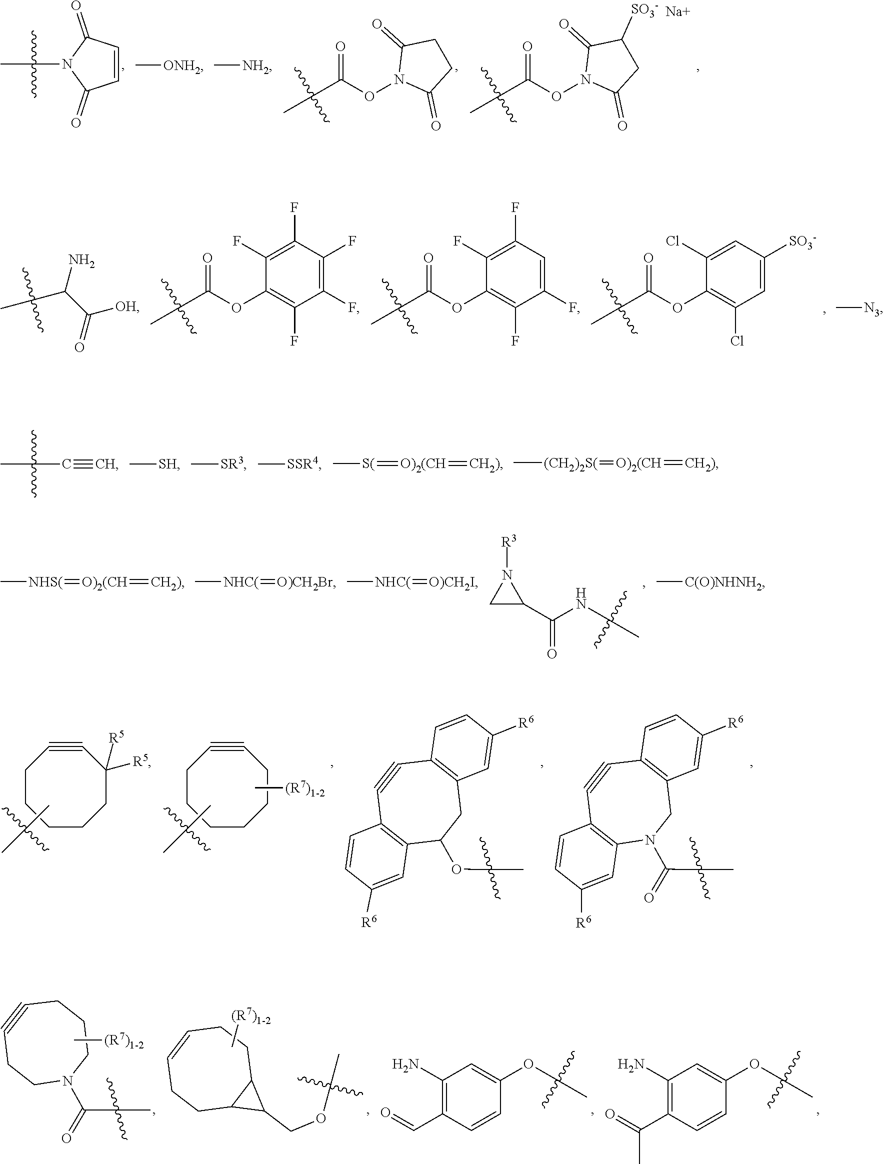

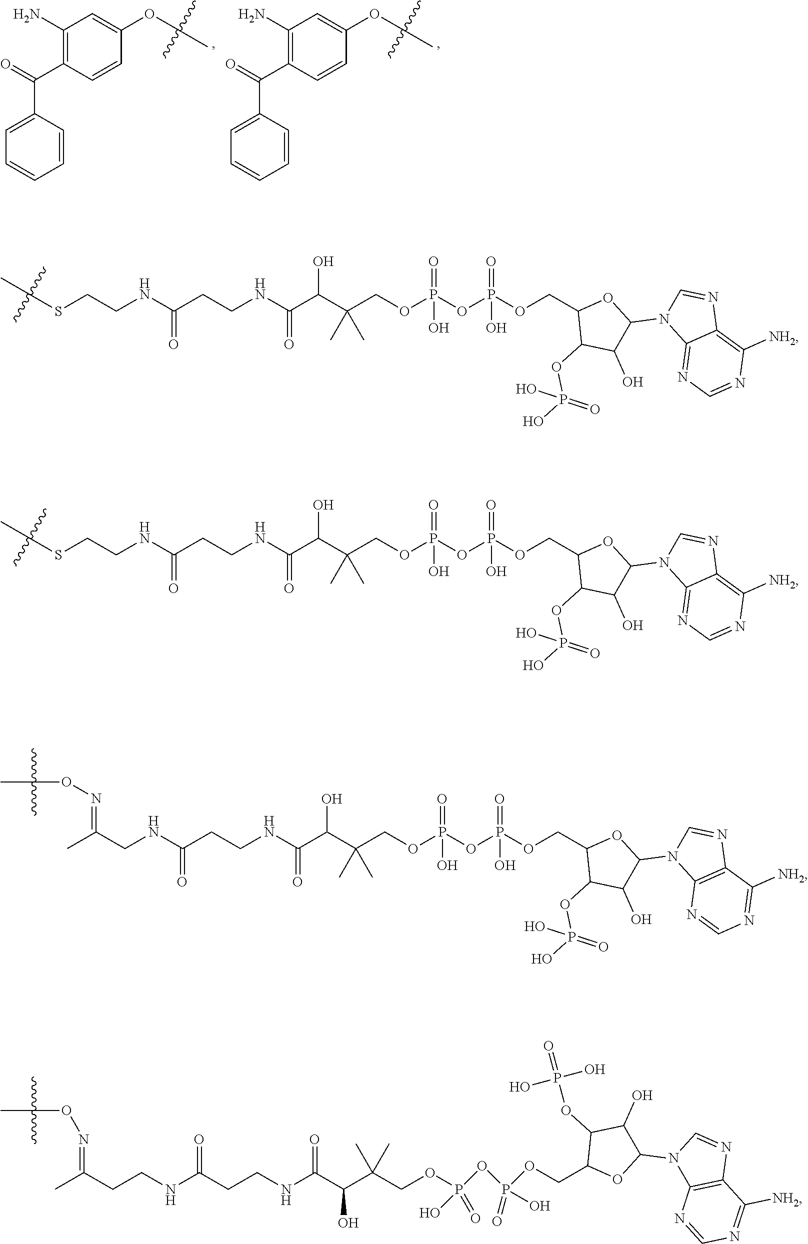 [SMILES: *Oc1ccc(C(=O)c2ccccc2)c(N)c1 ; *Oc1ccc(C(=O)c2ccccc2)c(N)c1 ; *SCCNC(=O)CCNC(=O)C(O)C(C)(C)COP(=O)(O)OP(=O)(O)OCC1OC(n2cnc3c(N)ncnc32)C(O)C1OP(=O)(O)O ; *SCCNC(=O)CCNC(=O)C(O)C(C)(C)COP(=O)(O)OP(=O)(O)OCC1OC(n2cnc3c(N)ncnc32)C(O)C1OP(=O)(O)O ; *O/N=C(\C)CNC(=O)CCNC(=O)C(O)C(C)(C)COP(=O)(O)OP(=O)(O)OCC1OC(n2cnc3c(N)ncnc32)C(O)C1OP(=O)(O)O ; *O/N=C(\C)CCNC(=O)CCNC(=O)[C@H](O)C(C)(C)COP(=O)(O)OP(=O)(O)OCC1OC(n2cnc3c(N)ncnc32)C(O)C1OP(=O)(O)O]

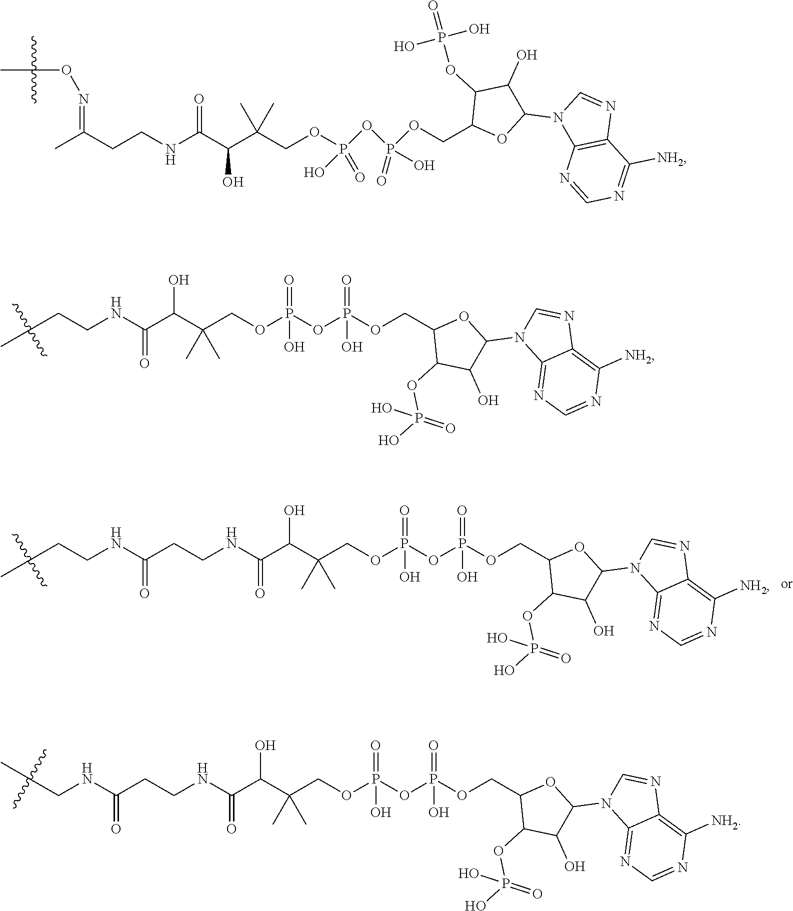 [SMILES: *O/N=C(\C)CCNC(=O)[C@H](O)C(C)(C)COP(=O)(O)OP(=O)(O)OCC1OC(n2cnc3c(N)ncnc32)C(O)C1OP(=O)(O)O ; *CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; "or" ; *CNC(=O)CCNC(=O)C(O)C(C)(C)COP(=O)(O)OP(=O)(O)OCC1OC(n2cnc3c(N)ncnc32)C(O)C1OP(=O)(O)O]

Embodiment 11. The compound of Formula (A′) or of any one of Embodiments 1 to 9, or pharmaceutically acceptable salt thereof, wherein:
R¹ is

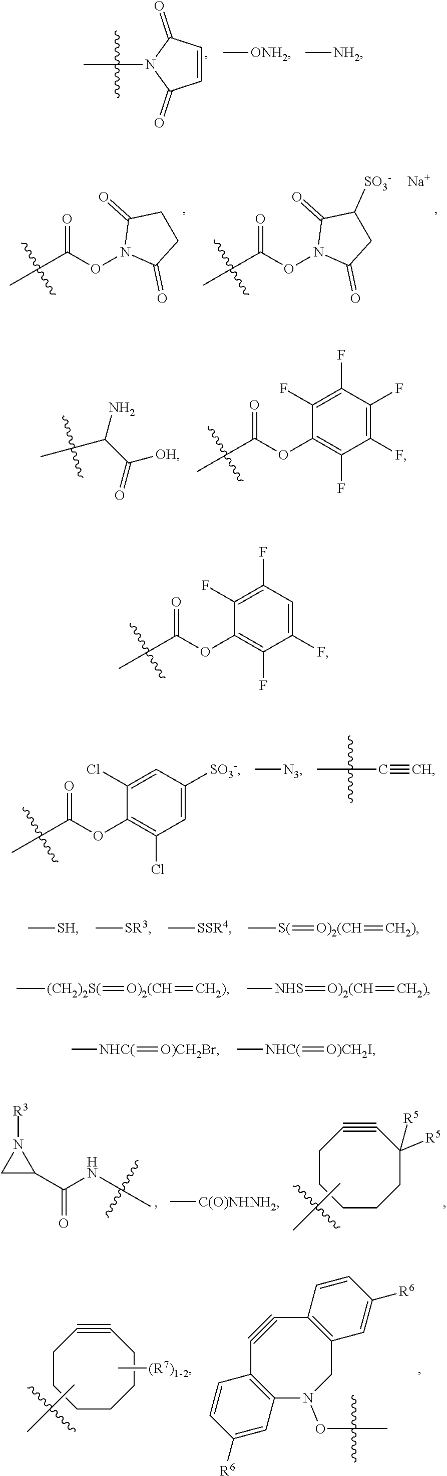

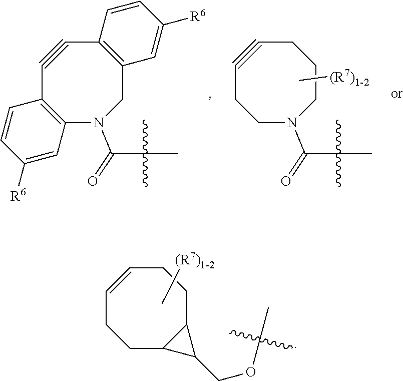

Embodiment 12. The compound of Formula (A′) or of any one of Embodiments 1 to 9, or pharmaceutically acceptable salt thereof, wherein:
R¹ is

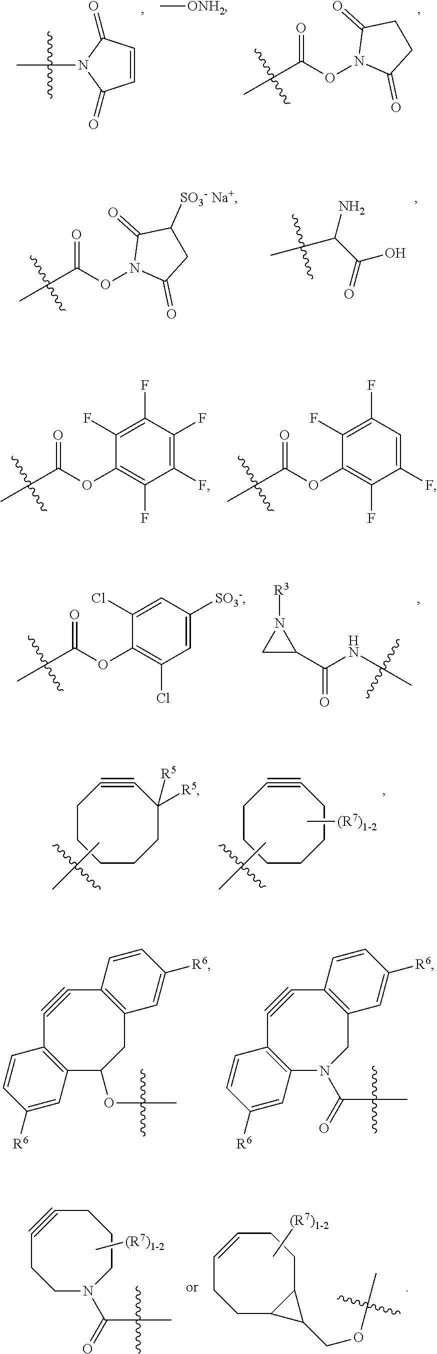

Embodiment 13. The compound of Formula (A′) or of any one of Embodiments 1 to 9, or pharmaceutically acceptable salt thereof, wherein:
R¹ is

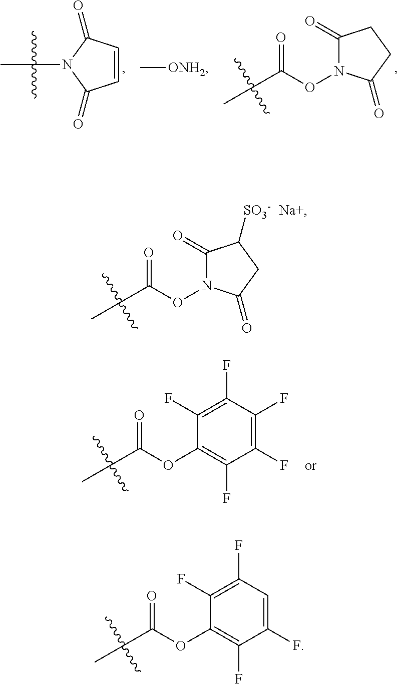

Embodiment 14. The compound of Formula (A′) or of any one of Embodiments 1 to 9, or pharmaceutically acceptable salt thereof, wherein R¹ is

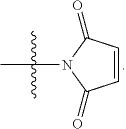 [SMILES: *N1C(=O)C=CC1=O]

Embodiment 15. The compound of Formula (A′) or of any one of Embodiments 1 to 9, or pharmaceutically acceptable salt thereof, wherein R¹ is —ONH₂.
Embodiment 16. The compound of Formula (A′) or of any one of Embodiments 1 to 9, or pharmaceutically acceptable salt thereof, wherein: R¹ is

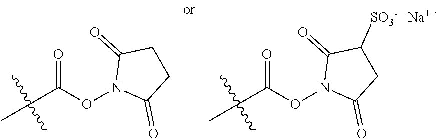

Embodiment 17. The compound of Formula (A′) or of any one of Embodiments 1 to 9, or pharmaceutically acceptable salt thereof, wherein:
R¹ is

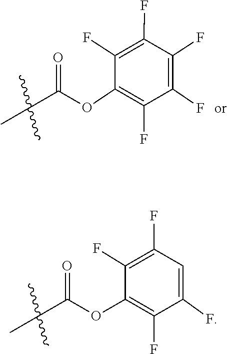 [SMILES: *C(=O)Oc1c(F)c(F)c(F)c(F)c1F ; *C(=O)Oc1c(F)c(F)cc(F)c1F]

Embodiment 18. The compound of Formula (A′) or of any one of Embodiments 1 to 9, or pharmaceutically acceptable salt thereof, having the structure:

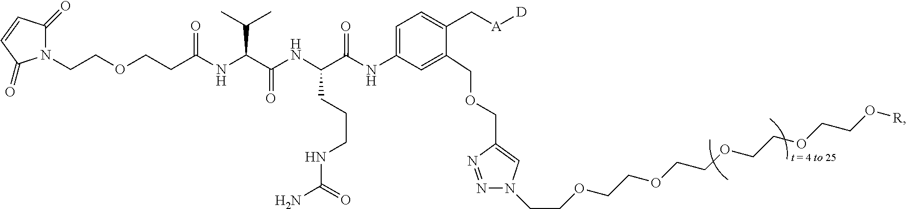

where R is H, —CH₃ or —CH₂CH₂C(═O)OH.
Embodiment 19. The compound of Formula (A′) or of any one of Embodiments 1 to 9, or pharmaceutically acceptable salt thereof, having the structure:

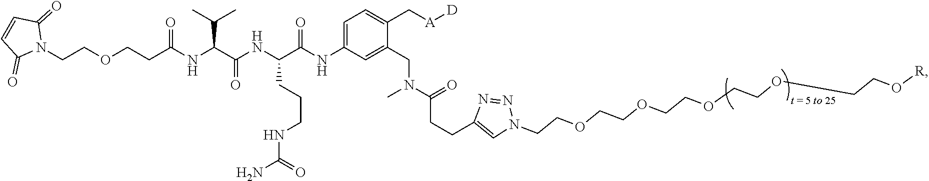

where R is H, —CH₃ or —CH₂CH₂C(═O)OH.
Embodiment 20. The compound of Formula (A′) or of any one of Embodiments 1 to 9, or pharmaceutically acceptable salt thereof, having the structure:

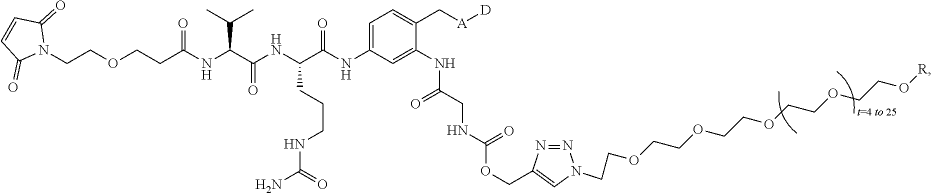

where
R is H, —CH₃ or —CH₂CH₂C(═O)OH.
   Embodiment 21. The compound of Formula (A′) or of any one of Embodiments 1 to 9, or pharmaceutically acceptable salt thereof, having the structure:

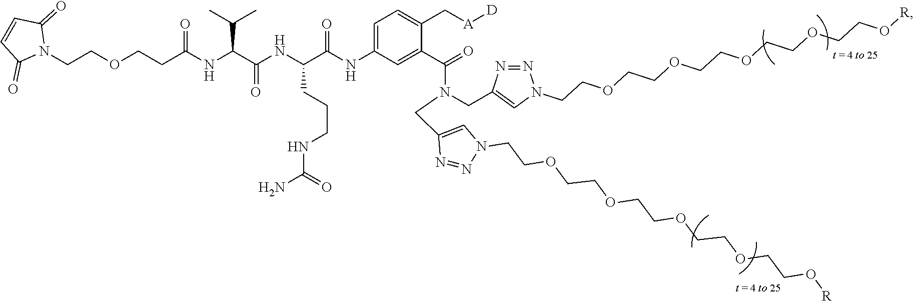

where
each R is independently selected from H, —CH₃ or —CH₂CH₂C(═O)OH.
   Embodiment 22. The compound of Formula (A′) or of any one of Embodiments 1 to 9, or pharmaceutically acceptable salt thereof, having the structure:

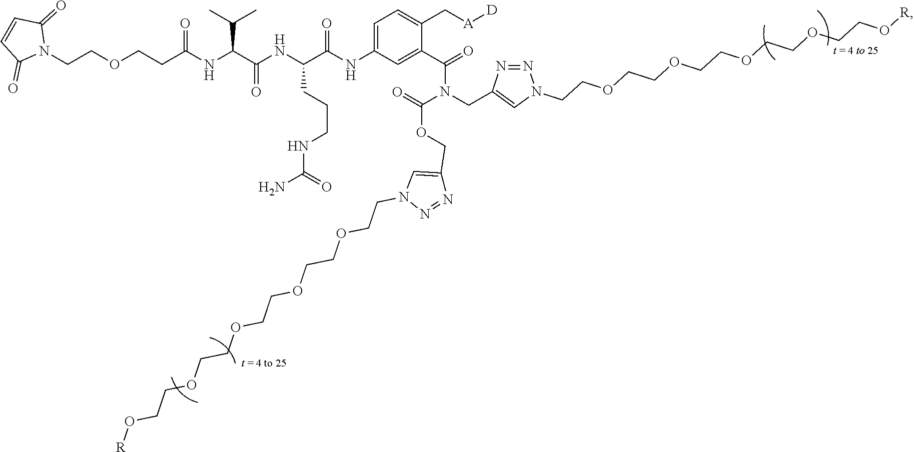

where
each R is independently selected from H, —CH₃ or —CH₂CH₂C(═O)OH.
   Embodiment 23. The compound of Formula (A′) or of any one of Embodiments 1 to 9 or pharmaceutically acceptable salt thereof, having the structure:

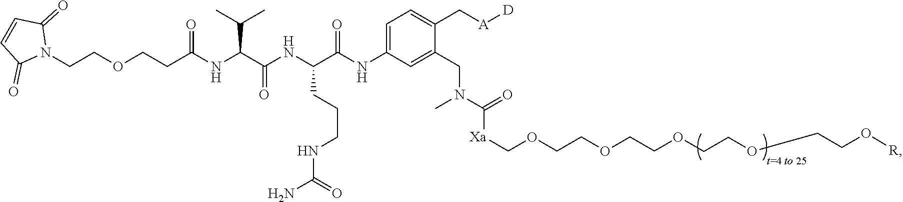

where
Xa is —CH₂—, —OCH₂—, —NHCH₂— or —NRCH₂— and each R independently is H, —CH₃ or —CH₂CH₂C(═O)OH.
   Embodiment 24. The compound of Formula (A′) or of any one of Embodiments 1 to 9, or pharmaceutically acceptable salt thereof, having the structure:

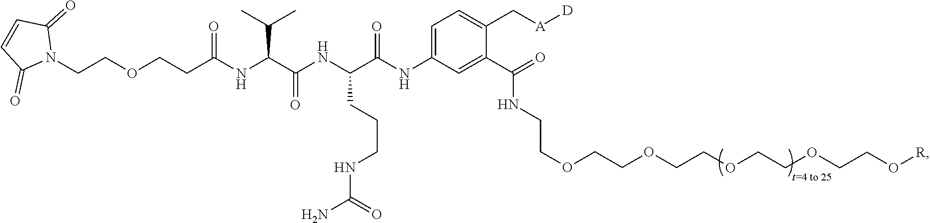

where R is H, —CH₃ or —CH₂CH₂C(═O)OH.
Embodiment 25. The compound of Formula (A′) or of any one of Embodiments 1 to 9, or pharmaceutically acceptable salt thereof, having the structure:

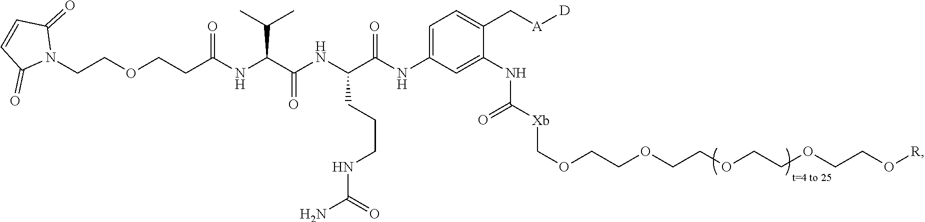

where
Xb is —CH₂—, —OCH₂—, —NHCH₂— or —NRCH₂— and each R independently is H, —CH₃ or —CH₂CH₂C(═O)OH.
   Embodiment 26. The compound of Formula (A′) or of any one of Embodiments 1 to 9, or pharmaceutically acceptable salt thereof, having the structure:

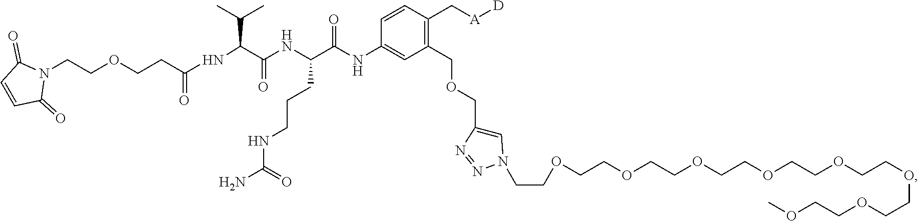

Embodiment 27. The compound of Formula (A′) or of any one of Embodiments 1 to 9, or pharmaceutically acceptable salt thereof, having the structure:

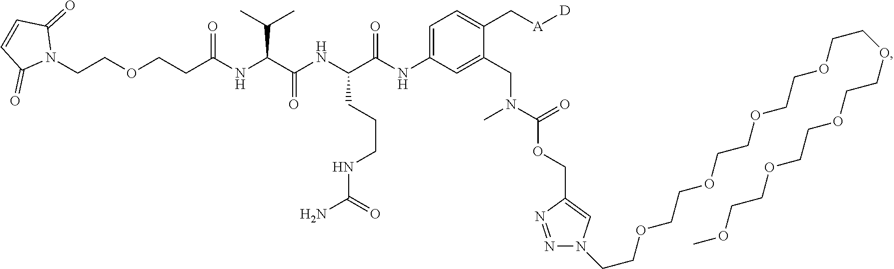

Embodiment 28. The compound of Formula (A′) or of any one of Embodiments 1 to 9, or pharmaceutically acceptable salt thereof, having the structure:

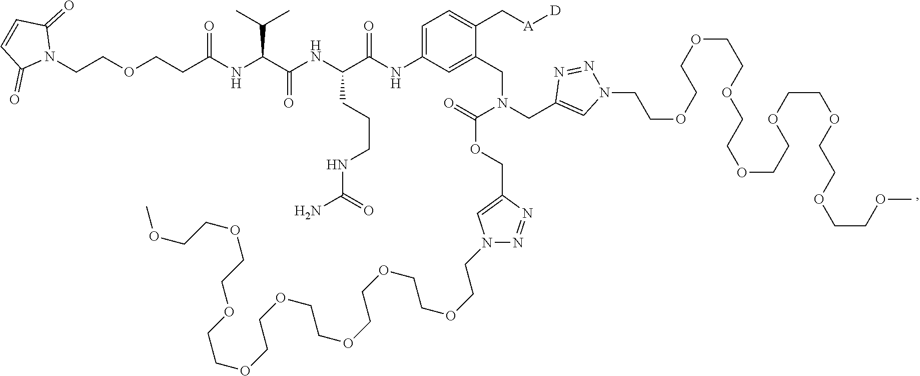

Embodiment 29. The compound of Formula (A′) or of any one of Embodiments 1 to 9, or pharmaceutically acceptable salt thereof, having the structure:

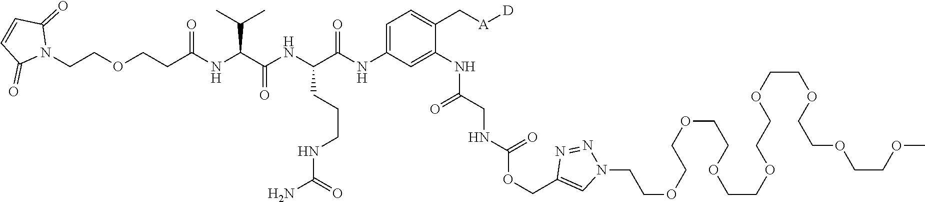

Embodiment 30. The compound of Formula (A′) or of any one of Embodiments 1 to 9, or pharmaceutically acceptable salt thereof, having the structure:

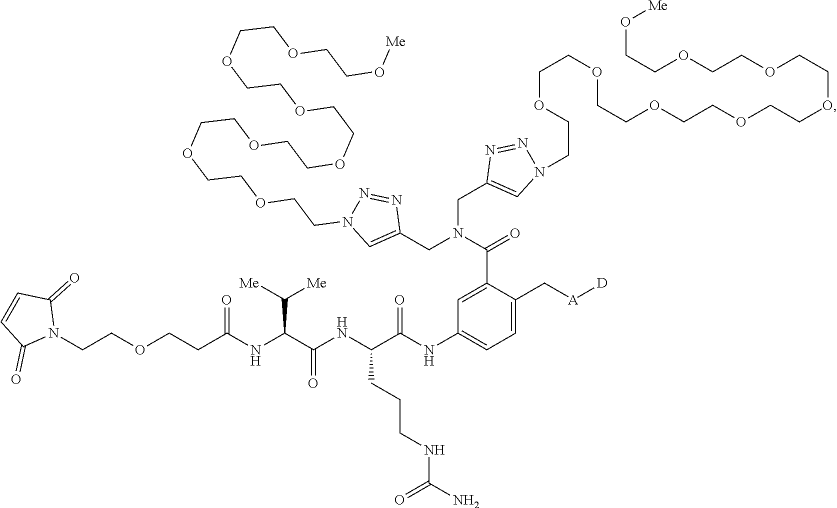

Embodiment 31. The compound of Formula (A′) or of any one of Embodiments 1 to 9, or pharmaceutically acceptable salt thereof, having the structure:

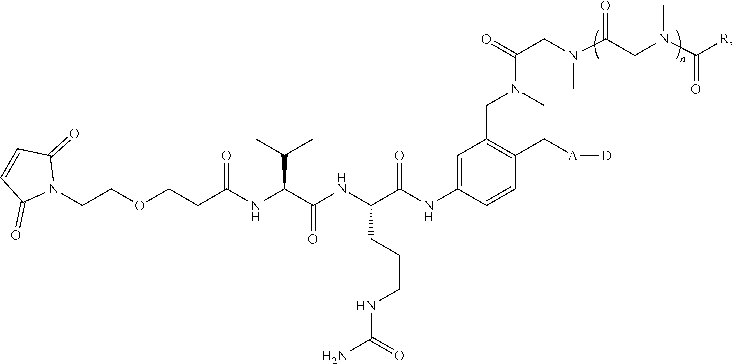

where n is an integer between 2 and 24.
Embodiment 32. The compound of Formula (A′) or of any one of Embodiments 1 to 9, or pharmaceutically acceptable salt thereof, having the structure of a compound in Table B.
Embodiment 33. A linker of the Linker-Drug group of Formula (A′) having the structure of Formula (C′),

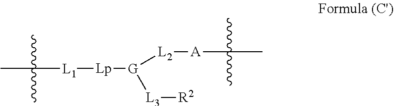

wherein
L₁ is a bridging spacer;
Lp is a bivalent peptide spacer;
G-L₂-A is a self-immolative spacer;
R² is a hydrophilic moiety;
L₂ is a bond, a methylene, a neopentylene or a C₂-C₃alkenylene;
A is a bond, —OC(═O)—*,

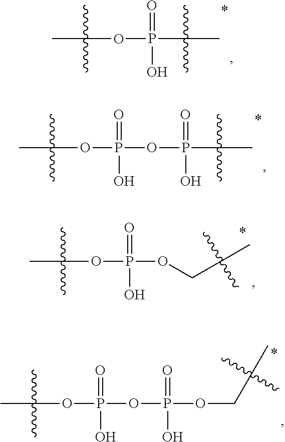 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D,
and
L₃ is a spacer moiety.
   Embodiment 34. The linker of Embodiment 33, wherein:
L₁ is a bridging spacer;
Lp is a bivalent peptide spacer comprising two to four amino acid residues;
G-L₂-A is a self-immolative spacer;
R² is a hydrophilic moiety;
L₂ is a bond, a methylene, a neopentylene or a C₂-C₃alkenylene;
A is a bond, —OC(═O)—*,

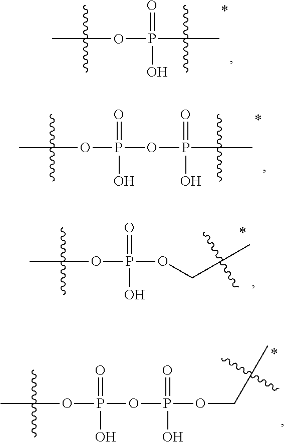 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D,
and
L₃ is a spacer moiety.
   Embodiment 35. The linker of Embodiment 33 or 34, wherein:
L₁ is a bridging spacer;
Lp is a bivalent peptide spacer comprising two to four amino acid residues;
the

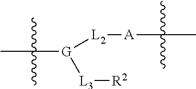

group is selected from:

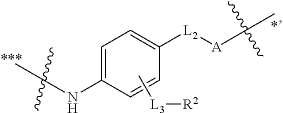

wherein the * of

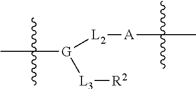

indicates the point of attachment to D (e.g., to an N or a O of the Drug moiety), the *** of

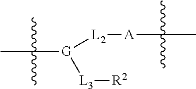

indicates the point of attachment to Lp;
R² is a hydrophilic moiety;
L₂ is a bond, a methylene, a neopentylene or a C₂-C₃alkenylene;
A is a bond, —OC(═O)—*,

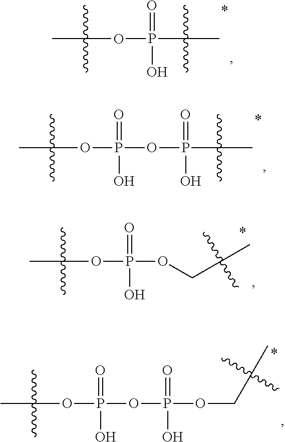 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D,
and
L₃ is a spacer moiety.
   Embodiment 36. The linker of any one of Embodiments 33 to 35, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R₃)₂(CH₂)ₘC(═O)NR₃(CH₂)ₘNR₃C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)(CH₂)ₘO)ₜ(CH₂)ₙX₁(CH₂)ₙ—*; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)(CH₂)ₘO)ₜ(CH₂)ₙC(═O)NH(CH₂)ₘ—**; *—C(═O)(CH₂)ₘC(R₃)₂—** or *—C(═O)(CH₂)ₘC(═O)NH(CH₂)ₘ—**, where the * of L₁ indicates the point of attachment to Lp;
R² is a hydrophilic moiety selected from polyethylene glycol, polyalkylene glycol, a sugar, an oligosaccharide, a polypeptide or C₂-C₆alkyl substituted with 1 to 3

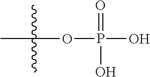 [SMILES: *OP(=O)(O)O]

groups;
each R³ is independently selected from H and C₁-C₆alkyl;
X₁ is

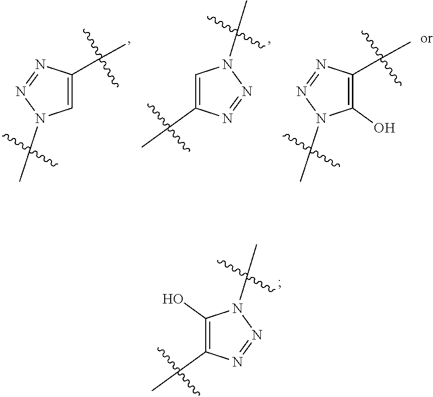

each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising an amino acid residue selected from glycine, valine, citrulline, lysine, isoleucine, phenylalanine, methionine, asparagine, proline, alanine, leucine, tryptophan, and tyrosine;
A is a bond, —OC(═O)—*,

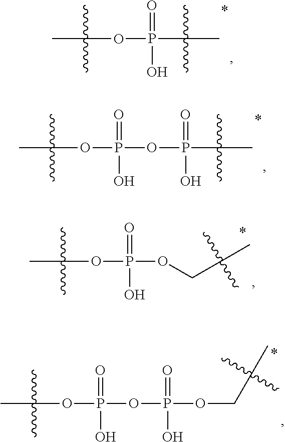 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety having the structure

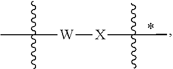

where
   W is —CH₂O—**, —CH₂N(R^{b})C(═O)O—**, —NHC(═O)C(R^{b})₂NHC(═O)O—** —NHC(═O)C(R^{b})₂NH—**, —NHC(═O)C(R^{b})₂NHC(═O)—**, —CH₂N(X—R₂)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—** —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
and
   the * of L₃ indicates the point of attachment to R².
      Embodiment 37. The linker of any one of Embodiments 33 to 36, wherein:
L₁ is *—C(═O)(CH₂)ₘO(CH₂)ₘ—**; *—C(═O)(CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

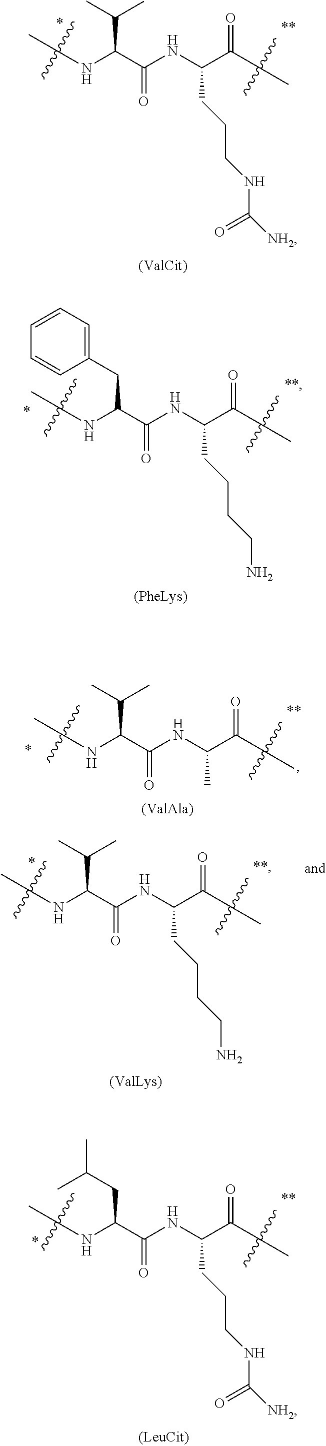

where the * of Lp indicates the attachment point to L₁;
L₃ is a spacer moiety having the structure

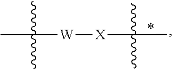

where
   W is —CH₂O—**, —CH₂N(R^{b})C(═O)O—**, —NHC(═O)CH₂NHC(═O)O—**, —NHC(═O)CH₂NH—**, —NHC(═O)CH₂NHC(═O)—**, —CH₂N(X—R₂)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

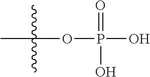 [SMILES: *OP(=O)(O)O]

groups;
and
A is a bond, —OC(═O)—*,

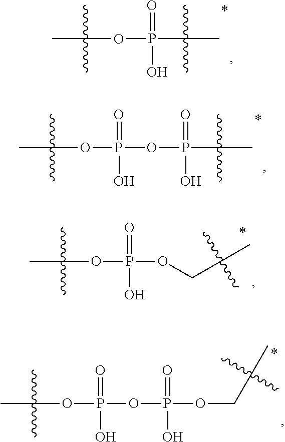 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D.
Embodiment 38. The linker of any one of Embodiments 33 to 37, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

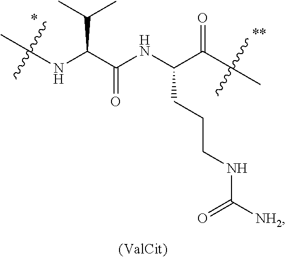 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

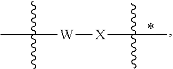

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

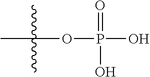 [SMILES: *OP(=O)(O)O]

groups;
and
A is a bond, —OC(═O)—*,

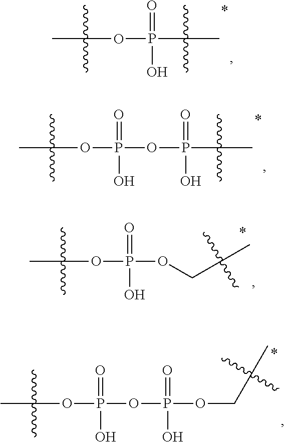 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D.
Embodiment 39. The linker of any one of Embodiments 33 to 38, wherein:
L₁ is *—C(═O)(CH₂)ₘO(CH₂)ₘ—*; *—C(═O)(CH₂)ₘO)ₜ(CH₂)ₙ—*; *—C(═O)(CH₂)ₘ—*; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

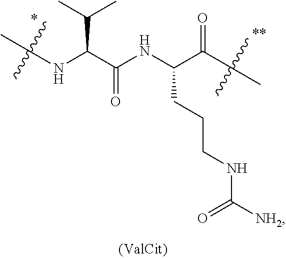 [SMILES: *N[C@H](C(=O)N[C@@H](CCCNC(N)=O)C(*)=O)C(C)C]

where the * of Lp indicates the attachment point to L₁;
L₃ is a spacer moiety having the structure

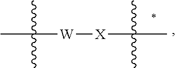

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —C(═O)NR^{b}—**, —C(═O)NH— **, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R_{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

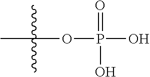 [SMILES: *OP(=O)(O)O]

groups;
and
A is a bond or —OC(═O) * in which * indicates the attachment point to D.
   Embodiment 40. The linker of any one of Embodiments 33 to 39, wherein:
L₁ is *—C(═O)(CH₂)ₘO(CH₂)ₘ—*; *—C(═O)(CH₂)ₘO)ₜ(CH₂)ₙ—**; *—C(═O)(CH₂)ₘ—*; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

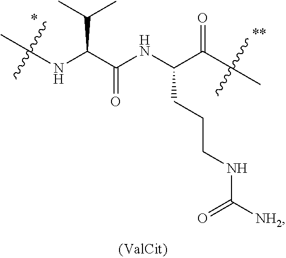 [SMILES: *N[C@H](C(=O)N[C@@H](CCCNC(N)=O)C(*)=O)C(C)C]

where the * of Lp indicates the attachment point to L₁;
L₃ is a spacer moiety having the structure

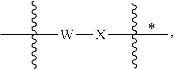

where
   W is —CH₂O—**, —CH₂N(R^{b})C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

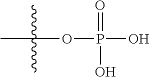 [SMILES: *OP(=O)(O)O]

groups;
and
A is a bond or —OC(═O) * in which * indicates the attachment point to D.
   Embodiment 41. The linker of Formula (C′) having the structure having the structure of Formula (D′),

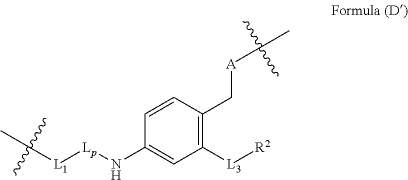

wherein

L₁ is a bridging spacer;

Lp is a bivalent peptide spacer;
R² is a hydrophilic moiety;
A is a bond, —OC(═O)—*,

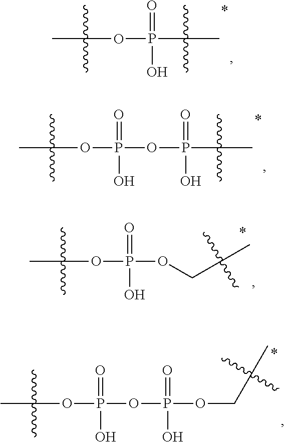 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D,
and
L₃ is a spacer moiety.
   Embodiment 42. The linker of Embodiments 41, wherein:
L₁ is a bridging spacer;
Lp is a bivalent peptide spacer comprising two to four amino acid residues;
R² is a hydrophilic moiety;
A is a bond, —OC(═O)—*,

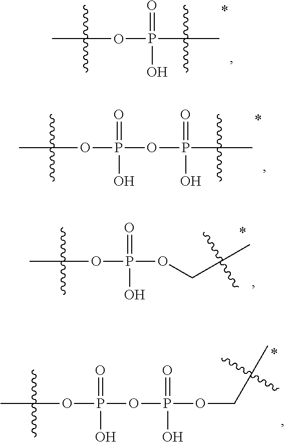 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D,
and
L₃ is a spacer moiety.
   Embodiment 43. The linker of Embodiment 41 or 42, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R₃)₂(CH₂)ₘC(═O)NR₃(CH₂)ₘNR₃C(═O)(CH₂)ₘ—**; *—C(═O)O(CH₂)ₘC(═O)NH(CH₂)ₘ—*; *—C(═O)(CH₂)ₘNH(CH₂)ₘ—*; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—*; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp;
R² is a hydrophilic moiety selected from polyethylene glycol, polyalkylene glycol, a sugar, an oligosaccharide, a polypeptide or C₂-C₆alkyl substituted with 1 to 3

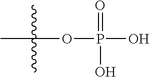 [SMILES: *OP(=O)(O)O]

groups;
each R³ is independently selected from H and C₁-C₆alkyl;
X₁ is

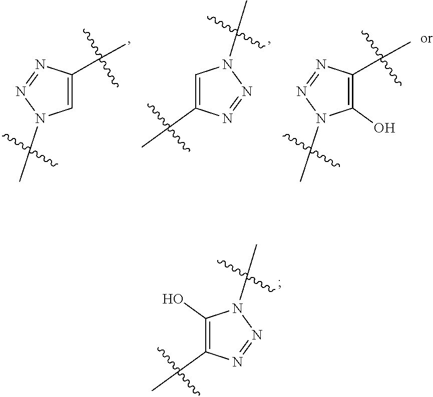

each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising an amino acid residue selected from glycine, valine, citrulline, lysine, isoleucine, phenylalanine, methionine, asparagine, proline, alanine, leucine, tryptophan, and tyrosine;
A is a bond, —OC(═O)—*,

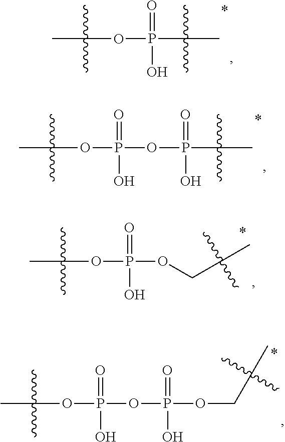 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety having the structure

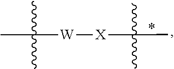

where
   W is —CH₂O—**, —CH₂N(R^{b})C(═O)O—**, —NHC(═O)CH₂NHC(═O)O—**, —CH₂N(X—R²)C(═O)O—*, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R².
      Embodiment 44. The linker of any one of Embodiments 41 to 43, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

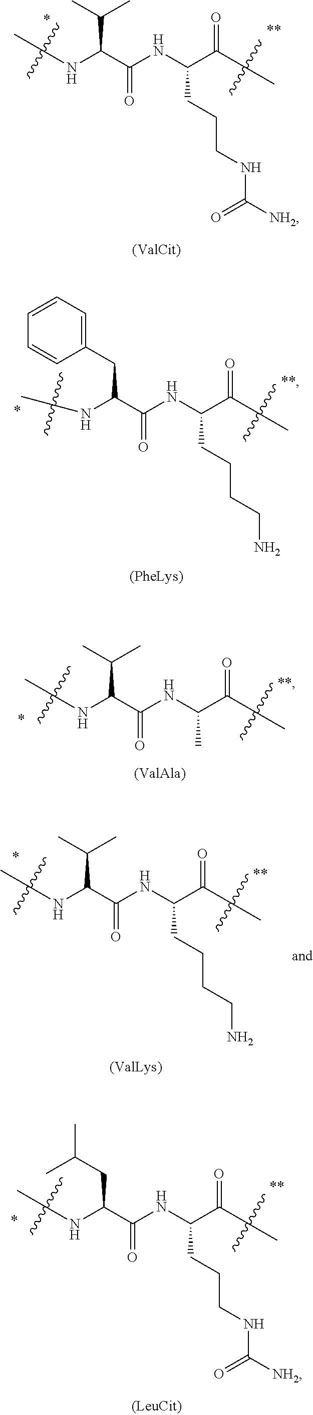

where the * of Lp indicates the attachment point to L₁ and the ** of Lp indicates the attachment point to the —NH— group of G;
L₃ is a spacer moiety having the structure

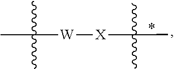

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

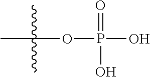 [SMILES: *OP(=O)(O)O]

groups;
and
A is a bond, —OC(═O)—*,

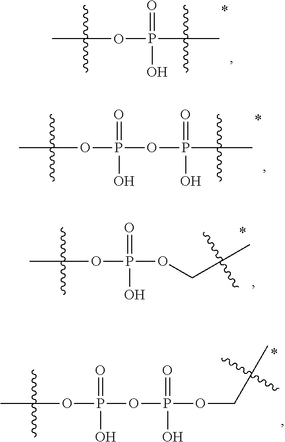 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D.
Embodiment 45. The linker of any one of Embodiments 41 to 44, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

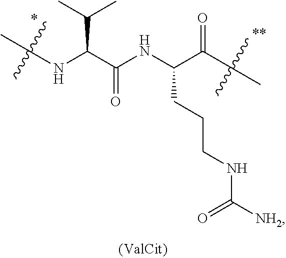 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

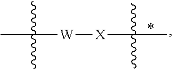

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

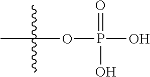 [SMILES: *OP(=O)(O)O]

groups;
and
A is a bond, —OC(═O)—*,

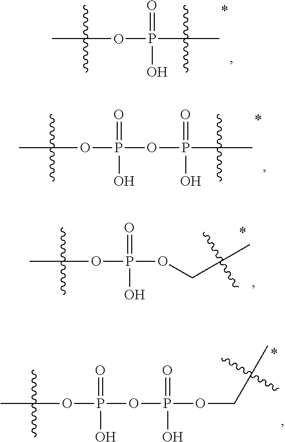 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D.
Embodiment 46. The linker of any one of Embodiments 41 to 45, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

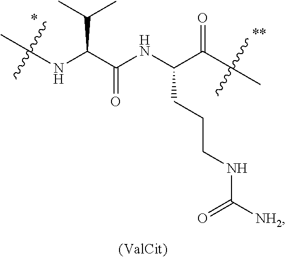 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

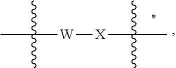

where
   W is —CH₂O—**, —CH₂N(R^{b})C(═O)O—**, —NHC(═O)CH₂NHC(═O)O—**, —CH₂N(X—R²)C(═O)O—*, —C(═O)N(X—R²)—*, —C(═O)NR^{b}—*, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R_{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

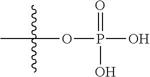 [SMILES: *OP(=O)(O)O]

groups;
and
A is a bond or —OC(═O)* in which * indicates the attachment point to D.
   Embodiment 47. The linker of any one of Embodiments 41 to 46, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

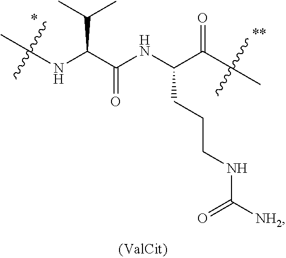 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

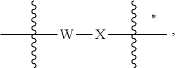

where
   W is —CH₂O—**, —CH₂N(R^{b})C(═O)O—**, —NHC(═O)CH₂NHC(═O)O—** —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²;
   and
   the * of L₃ indicates the point of attachment to R²;

R² is a hydrophilic moiety selected from polyethylene glycol, polyalkylene glycol, a sugar, an oligosaccharide, a polypeptide or C₂-C₆alkyl substituted with 1 to 3

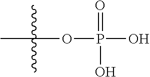 [SMILES: *OP(=O)(O)O]

groups;
and
A is a bond or —OC(═O)* in which * indicates the attachment point to D.
   Embodiment 48. The linker of any one of Embodiments 33 to 47, having the structure:

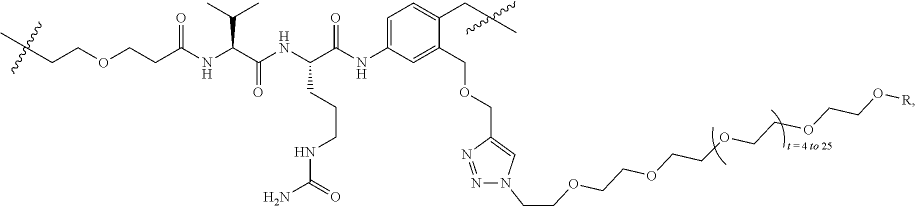

where R is H, —CH₃ or —CH₂CH₂C(═O)OH.
Embodiment 49. The linker of any one of Embodiments 33 to 47, having the structure:

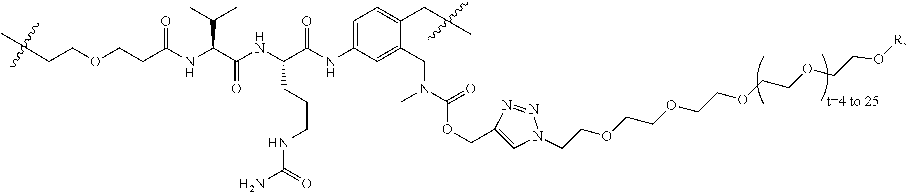

where R is H, —CH₃ or —CH₂CH₂C(═O)OH.
Embodiment 50. The linker of any one of Embodiments 33 to 47, having the structure:

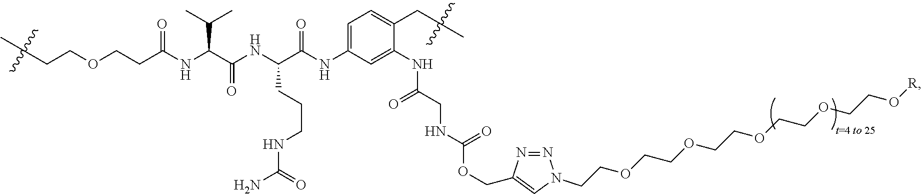

where R is H, —CH₃ or —CH₂CH₂C(═O)OH.
Embodiment 51. The linker of any one of Embodiments 33 to 47, having the structure:

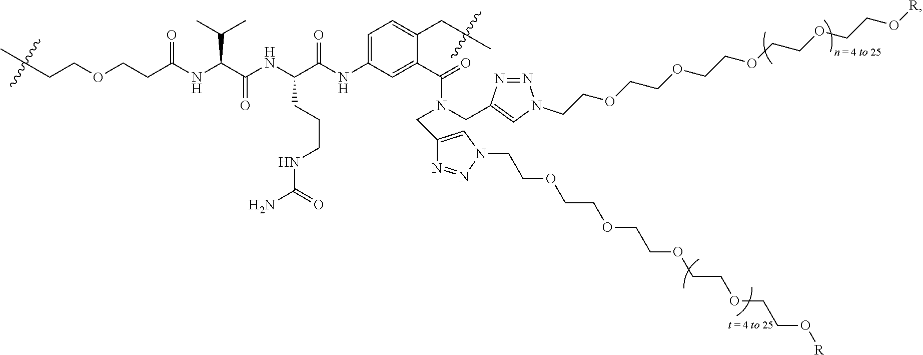

where
each R is independently selected from H, —CH₃ or —CH₂CH₂C(═O)OH.
   Embodiment 52. The linker of any one of Embodiments 37 to 47, having the structure:

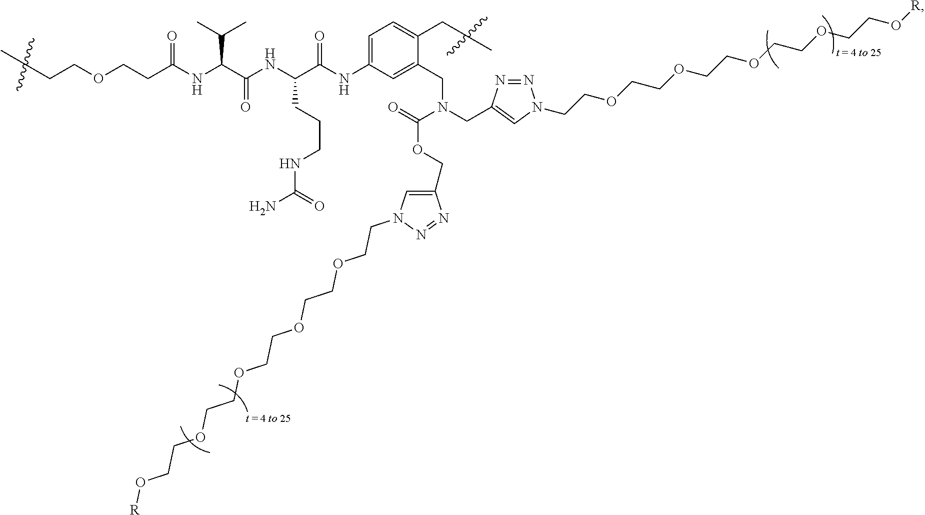

where
each R is independently selected from H, —CH₃ or —CH₂CH₂C(═O)OH.
   Embodiment 53. The linker of any one of Embodiments 33 to 47, having the structure:

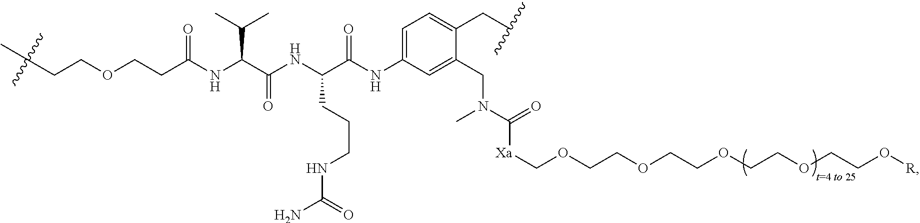

where
Xa is —CH₂—, —OCH₂—, —NHCH₂— or —NRCH₂— and each R independently is H, —CH₃ or —CH₂CH₂C(═O)OH.
   Embodiment 54. The linker of any one of Embodiments 33 to 47, having the structure:

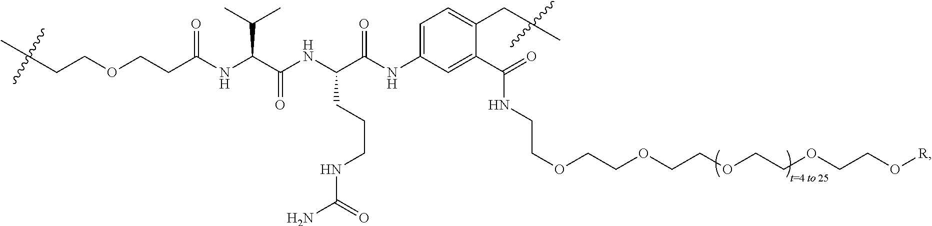

where
R is H, —CH₃ or —CH₂CH₂C(═O)OH.
   Embodiment 55. The linker of any one of Embodiments 33 to 47, having the structure:

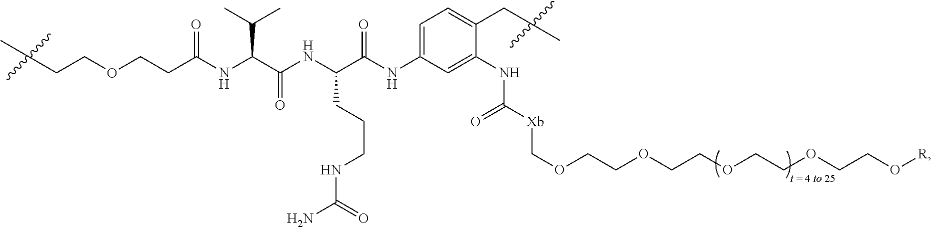

where
Xb is —CH₂—, —OCH₂—, —NHCH₂— or —NRCH₂— and each R independently is H, —CH₃ or —CH₂CH₂C(═O)OH.
   Embodiment 56. The linker of any one of Embodiments 33 to 47, having the structure:

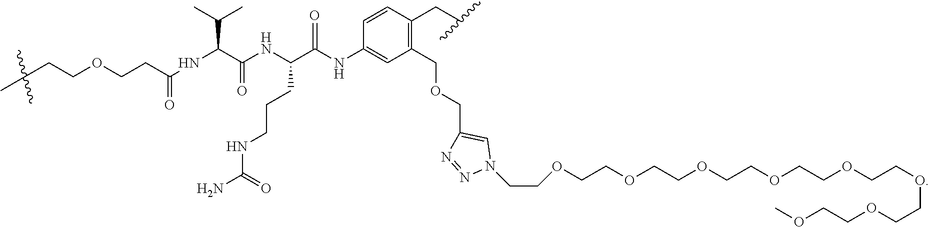 [SMILES: *CCOCCC(=O)N[C@H](C(=O)N[C@@H](CCCNC(N)=O)C(=O)Nc1ccc(C*)c(COCc2cn(CCOCCOCCOCCOCCOCCOCCOCCOC)nn2)c1)C(C)C]

Embodiment 57. The linker of any one of Embodiments 33 to 47, having the structure:

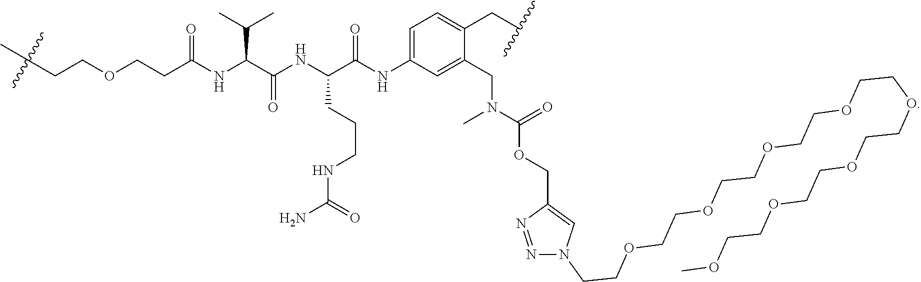 [SMILES: *CCOCCC(=O)N[C@H](C(=O)N[C@@H](CCCNC(N)=O)C(=O)Nc1ccc(C*)c(CN(C)C(=O)OCc2cn(CCOCCOCCOCCOCCOCCOCCOCCOC)nn2)c1)C(C)C]

Embodiment 58. The linker of any one of Embodiments 37 to 47, having the structure:

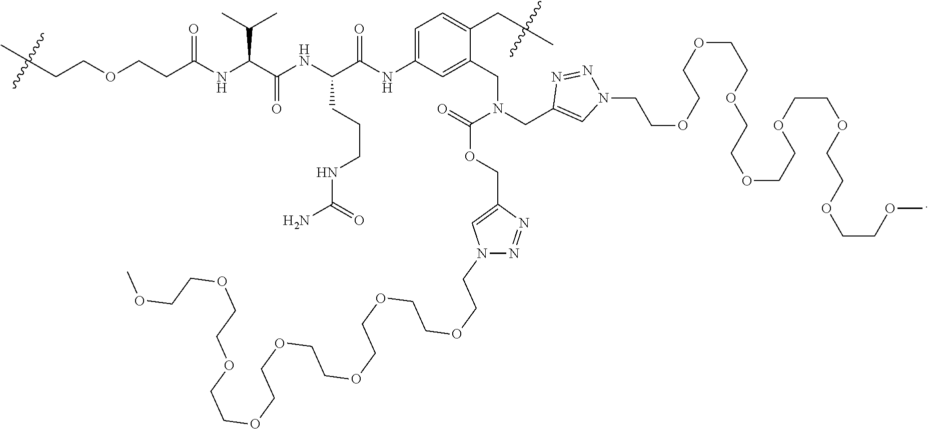 [SMILES: *CCOCCC(=O)N[C@H](C(=O)N[C@@H](CCCNC(N)=O)C(=O)Nc1ccc(C*)c(CN(Cc2cn(CCOCCOCCOCCOCCOCCOCCOCCOC)nn2)C(=O)OCc2cn(CCOCCOCCOCCOCCOCCOCCOCCOC)nn2)c1)C(C)C]

Embodiment 59. The linker of any one of Embodiments 33 to 47, having the structure:

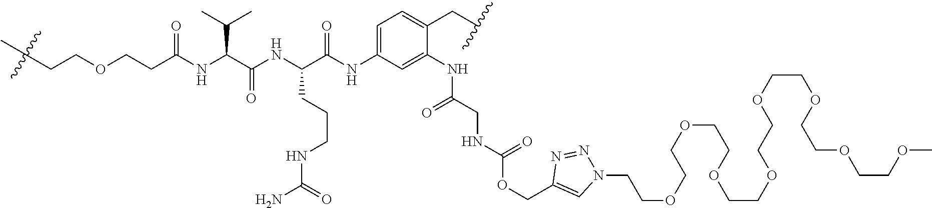 [SMILES: *CCOCCC(=O)N[C@H](C(=O)N[C@@H](CCCNC(N)=O)C(=O)Nc1ccc(C*)c(NC(=O)CNC(=O)OCc2cn(CCOCCOCCOCCOCCOCCOCCOCCOC)nn2)c1)C(C)C]

Embodiment 60. The linker of any one of Embodiments 33 to 47, having the structure:

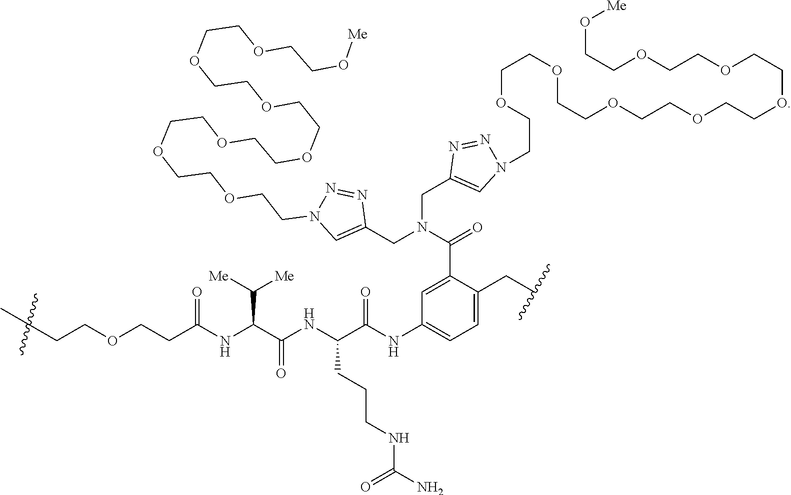 [SMILES: *CCOCCC(=O)N[C@H](C(=O)N[C@@H](CCCNC(N)=O)C(=O)Nc1ccc(C*)c(C(=O)N(Cc2cn(CCOCCOCCOCCOCCOCCOCCOCCOC)nn2)Cc2cn(CCOCCOCCOCCOCCOCCOCCOCCOC)nn2)c1)C(C)C]

Embodiment 61. The linker of any one of Embodiments 33 to 47, having the structure:

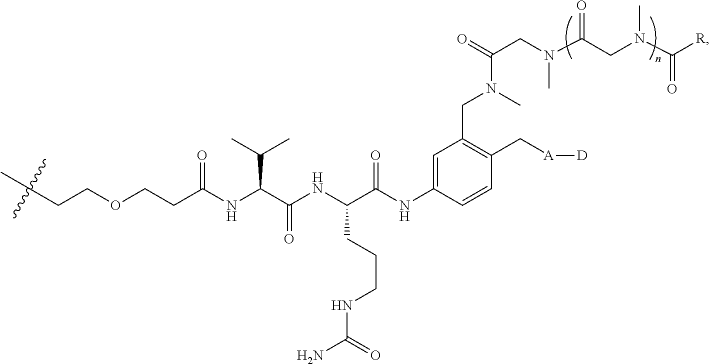

where n is an integer between 2 and 24

For illustrative purposes, the general reaction schemes depicted herein provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

By way of example, a general synthesis for compounds of Formula (B′) is shown below in Scheme 1.

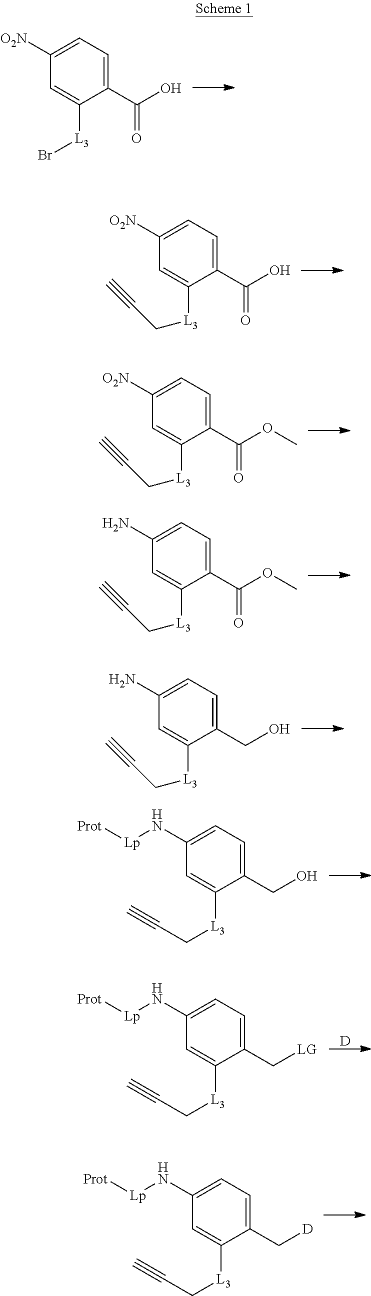

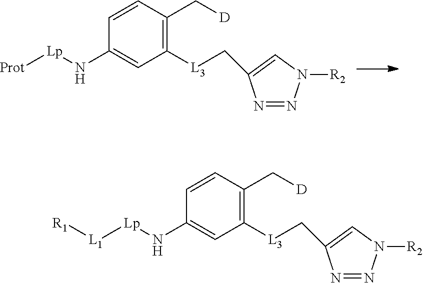

### Antibody Drug Conjugates of the Invention

The present invention provides Antibody Drug Conjugates, also referred to herein as immunoconjugates, which comprise linkers which comprise one or more hydrophilic moieties.

The Antibody Drug Conjugates of the invention have the structure of Formula (E′):

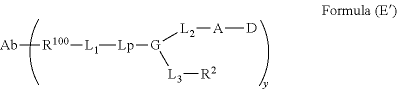

wherein:
Ab is an anti-EphA2 antibody or fragment thereof;
R¹⁰⁰ is a coupling group;
L₁ is a bridging spacer;
Lp is a bivalent peptide spacer;
G-L₂-A is a self-immolative spacer;
R² is a hydrophilic moiety;
L₂ is a bond, a methylene, a neopentylene or a C₂-C₃ alkenylene;
A is a bond, —OC(═O)—*,

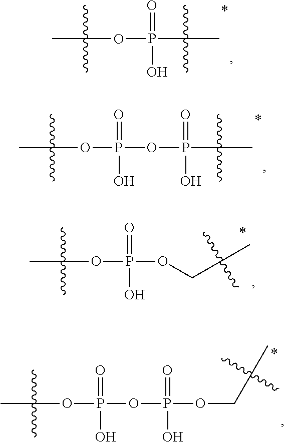 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety;
D is a Drug moiety as defined herein, e.g., a Bcl-XL inhibitor, and may comprise an N, wherein D can be connected to A via a direct bond from A to the N of the Drug moiety,
and
y is 1, 2, 3, 4, 5, 6, 7, 8, 9, 10, 11, 12, 13, 14, 15 or 16.

Certain aspects and examples of the Antibody Drug Conjugates of the invention are provided in the following listing of enumerated embodiments. It will be recognized that features specified in each embodiment may be combined with other specified features to provide further embodiments of the present invention.

Embodiment 62. The immunoconjugate of Formula (E′) wherein:
Ab is an anti-EphA2 antibody or fragment thereof;
R¹⁰⁰ is a coupling group;
L₁ is a bridging spacer;
Lp is a bivalent peptide spacer comprising two to four amino acid residues;
G-L₂-A is a self-immolative spacer;
R² is a hydrophilic moiety;
L₂ is a bond, a methylene, a neopentylene or a C₂-C₃alkenylene;
A is a bond, —OC(═O)—*,

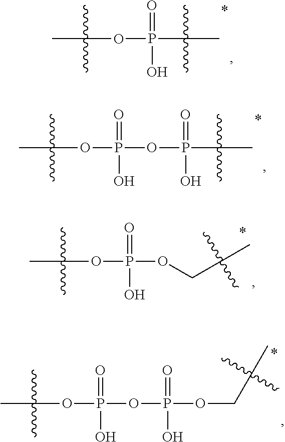 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety;
D is a Drug moiety as defined herein wherein D is connected to A via a direct bond from A to D (e.g., an N of the Drug moiety),
and
y is 1, 2, 3, 4, 5, 6, 7, 8, 9, 10, 11, 12, 13, 14, 15 or 16.
   Embodiment 63. The immunoconjugate of Formula (E′) or Embodiment 62, wherein:
Ab is an anti-EphA2 antibody or fragment thereof;
R¹⁰⁰ is a coupling group;
L₁ is a bridging spacer;
Lp is a bivalent peptide spacer comprising two to four amino acid residues;
the

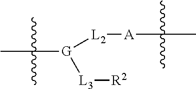

group is selected from:

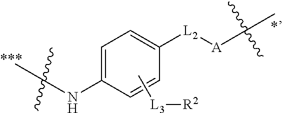

wherein the * of

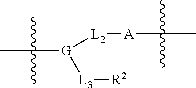

indicates the point of attachment to D (e.g., to an N or a O of the Drug moiety), the *** of

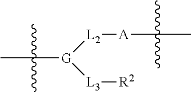

indicates the point of attachment to Lp;
R² is a hydrophilic moiety;
L₂ is a bond, a methylene, a neopentylene or a C₂-C₃alkenylene;

A is a bond, —OC(═O)—*,

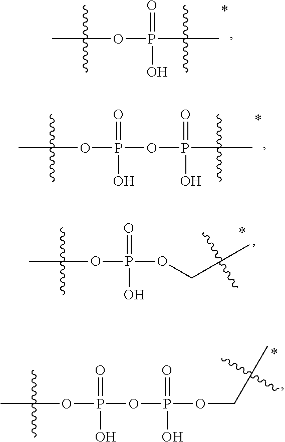 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety;
D is a Drug moiety as defined herein and comprising an N, wherein D is connected to A via a direct bond from A to the N of the Drug moiety,
and
y is 1, 2, 3, 4, 5, 6, 7, 8, 9, 10, 11, 12, 13, 14, 15 or 16.
   Embodiment 64. The immunoconjugate of Formula (E′) or any one of Embodiments 62 to 63 having the structure of Formula (F′),

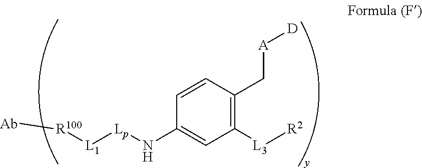

wherein:
Ab is an anti-EphA2 antibody or fragment thereof;
R¹⁰⁰ is a coupling group;
L₁ is a bridging spacer;
Lp is a bivalent peptide spacer comprising two to four amino acid residues;
R² is a hydrophilic moiety;
A is a bond, —OC(═O)—*,

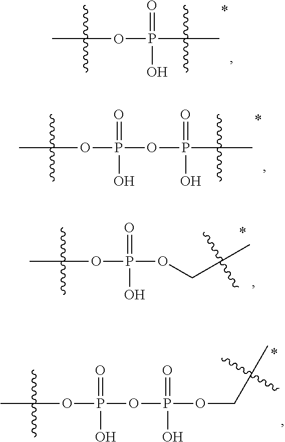 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety;
D is a Drug moiety as defined herein and comprising an N, wherein D is connected to A via a direct bond from A to the N of the Drug moiety,
and
y is 1, 2, 3, 4, 5, 6, 7, 8, 9, 10, 11, 12, 13, 14, 15 or 16.
   Embodiment 65. The immunoconjugate of Formula (D′) or any one of Embodiments 62 to 64, wherein:
Ab is an anti-EphA2 antibody or fragment thereof;
R¹⁰⁰ is

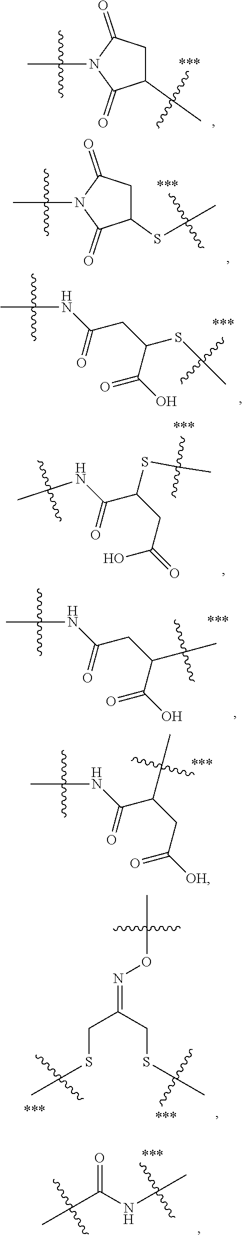

—S—, —C(═O)—, —ON═***, —NHC(═O)CH₂—***, —S(═O)₂CH₂CH₂—***, —(CH₂)₂S(═O)₂CH₂CH₂—***, —NHS(═O)₂CH₂CH₂—***, —NHC(═O)CH₂CH₂—***, —CH₂NHCH₂CH₂—***, —NHCH₂CH₂—***,

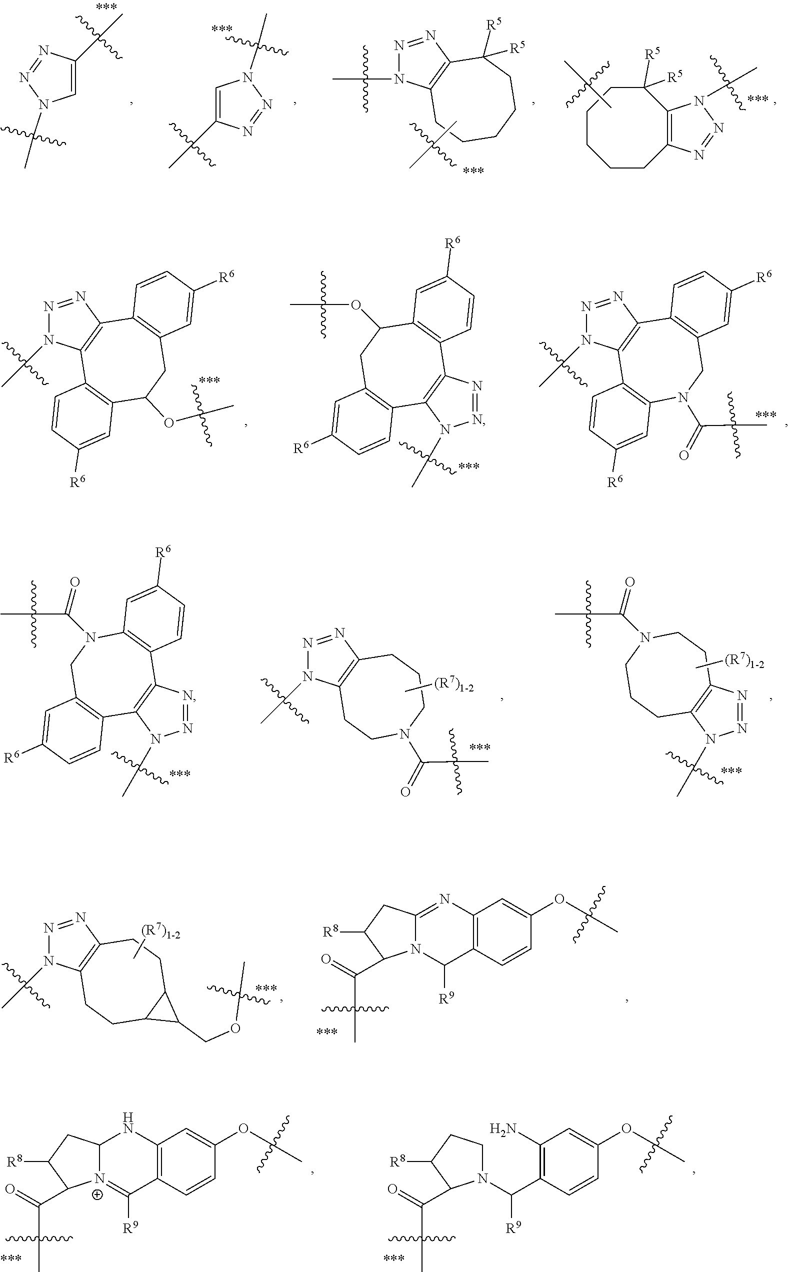

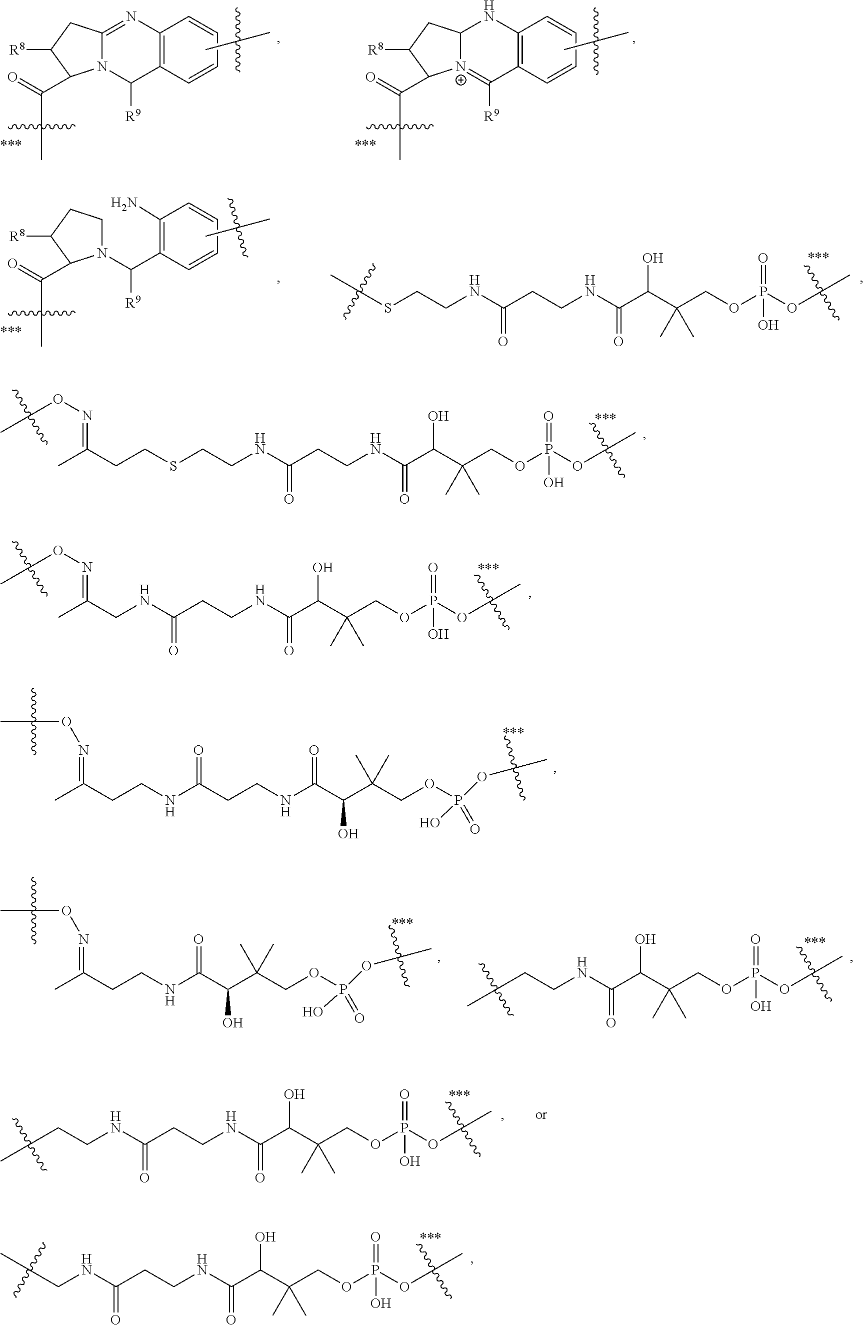

where the *** of R¹⁰⁰ indicates the point of attachment to Ab;
L₁ is *—C(═O)(CH₂)ₘO(CH₂)ₘ—**; *—C(═O)(CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R₃)₂(CH₂)ₘC(═O)NR₃(CH₂)ₘNR₃C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)(CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R₃)₂—** or *—C(═O)(CH₂)ₘC(═O)NH(CH₂)ₘ—**, where the * of L₁ indicates the point of attachment to Lp, and the ** of L₁ indicates the point of attachment to R¹⁰⁰;
R² is a hydrophilic moiety selected from polyethylene glycol, polyalkylene glycol, a sugar, an oligosaccharide, a polypeptide or C₂-C₆alkyl substituted with 1 to 3

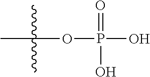 [SMILES: *OP(=O)(O)O]

groups;
each R³ is independently selected from H and C₁-C₆alkyl;
R⁴ is 2-pyridyl or 4-pyridyl;
each R⁵ is independently selected from H, C₁-C₆alkyl, F, Cl, and —OH;
each R⁶ is independently selected from H, C₁-C₆alkyl, F, Cl, —NH₂, —OCH₃, —OCH₂CH₃, —N(CH₃)₂, —CN, —NO₂ and —OH;
each R⁷ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
X₁ is

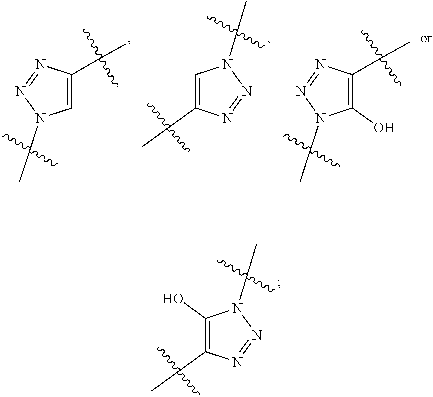

each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising an amino acid residue selected from valine, citrulline, lysine, isoleucine, phenylalanine, methionine, asparagine, proline, alanine, leucine, tryptophan, and tyrosine;
A is a bond, —OC(═O)—*,

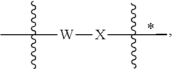

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety having the structure

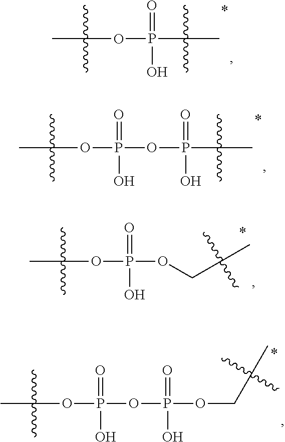

where
   W is —CH₂O—**, —CH₂N(R^{b})C(═O)O—**, —NHC(═O)C(R^{b})₂NHC(═O)O—** —NHC(═O)C(R^{b})₂NH—**, —NHC(═O)C(R^{b})₂NHC(═O)—**, —CH₂N(X—R₂)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
D is a Drug moiety as defined herein and comprising an N, wherein D is connected to A via a direct bond from A to the N of the Drug moiety,
and
y is 1, 2, 3, 4, 5, 6, 7, 8, 9, 10, 11, 12, 13, 14, 15 or 16.
   Embodiment 66. The immunoconjugate of Formula (D′) or any one of Embodiments 62 to 65, wherein:
Ab is an anti-EphA2 antibody or fragment thereof;
R¹⁰⁰ is

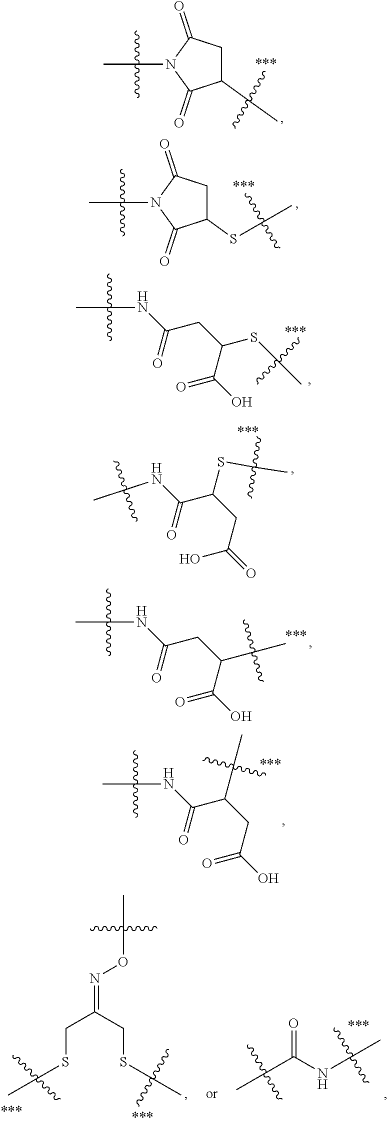

where the *** of R¹⁰⁰ indicates the point of attachment to Ab;
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹⁰⁰;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

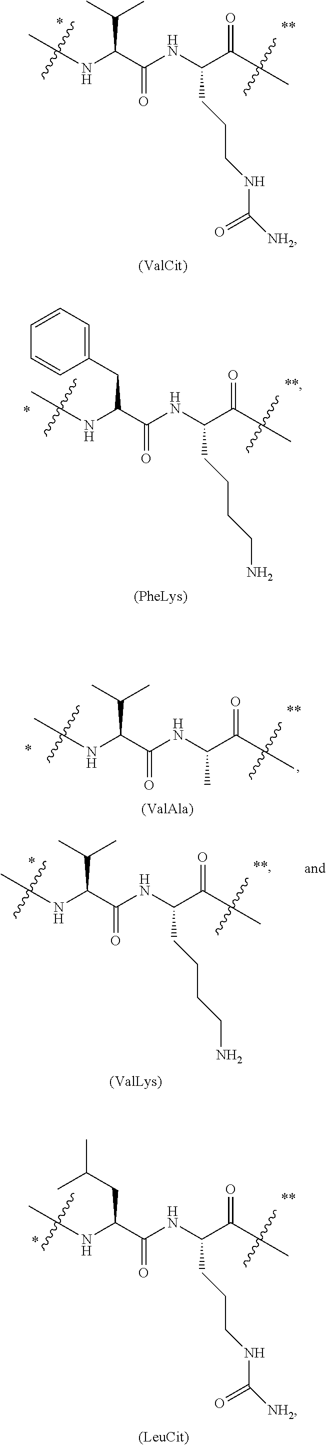

where the * of Lp indicates the attachment point to L₁ and the ** of Lp indicates the attachment point to the —NH— group of G;
L₃ is a spacer moiety having the structure

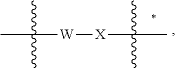

where
   W is —CH₂O—**, —CH₂N(R^{b})C(═O)O—**, —NHC(═O)CH₂NHC(═O)O—** —NHC(═O)CH₂NH—**, —NHC(═O)CH₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

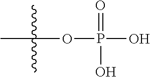 [SMILES: *OP(=O)(O)O]

groups;
A is a bond, —OC(═O)—*,

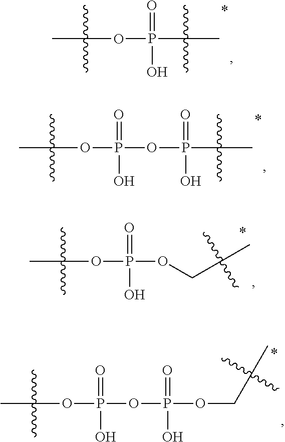 [SMILES: *OP(*)(=O)O ; *OP(=O)(O)OP(*)(=O)O ; *COP(=O)(O)O* ; *COP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
D is a Drug moiety as defined herein and comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.
   Embodiment 67. The immunoconjugate of Formula (E′) or any one of Embodiments 62 to 66, wherein:
Ab is an anti-EphA2 antibody or fragment thereof;
R¹⁰⁰ is

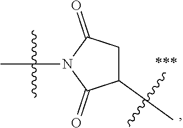 [SMILES: *C1CC(=O)N(*)C1=O]

where the *** of R¹⁰⁰ indicates the point of attachment to Ab;
L₁ is *—C(═O)(CH₂)ₘO(CH₂)ₘ—**; *—C(═O)(CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹⁰⁰;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

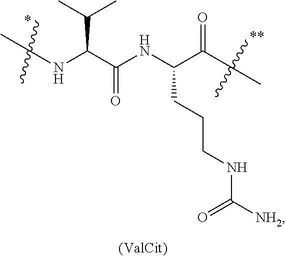 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

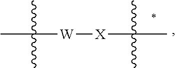

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3 A is a bond, —OC(═O)—*,

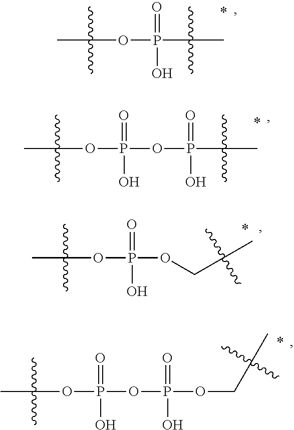

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
D is a Drug moiety as defined herein and comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.
   Embodiment 68. The immunoconjugate of Formula (E′) or any one of Embodiments 62 to 67, wherein:
Ab is an anti-EphA2 antibody or fragment thereof;
R¹⁰⁰ is

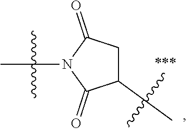 [SMILES: *C1CC(=O)N(*)C1=O]

where the *** of R¹⁰⁰ indicates the point of attachment to Ab;
L₁ is *—C(═O)(CH₂)ₘO(CH₂)ₘ—**; *—C(═O)(CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹⁰⁰;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

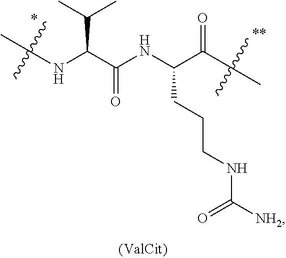 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

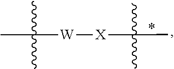

where
   W is —CH₂O—**, —CH₂N(R^{b})C(═O)O—**, —NHC(═O)CH₂NHC(═O)O—** —CH₂N(X—R²)C(═O)O—**, —C(═O)N(X—R²)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

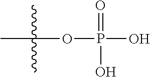 [SMILES: *OP(=O)(O)O]

groups;
A is a bond or —OC(═O) * in which * indicates the attachment point to D;
D is a Drug moiety as defined herein and comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.
   Embodiment 69. The immunoconjugate of Formula (E′) or any one of Embodiments 62 to 68, wherein:
Ab is an anti-EphA2 antibody or fragment thereof;
R¹⁰⁰ is

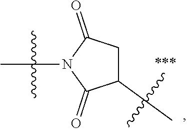 [SMILES: *C1CC(=O)N(*)C1=O]

where the *** of R¹⁰⁰ indicates the point of attachment to Ab;
L₁ is *—C(═O)(CH₂)ₘO(CH₂)ₘ—*; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹⁰⁰;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

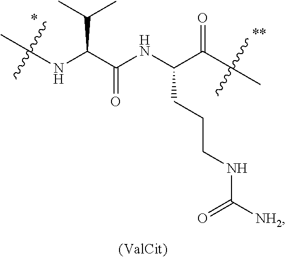 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

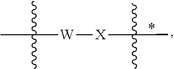

where
   W is —CH₂O—**, —CH₂N(R^{b})C(═O)O—**, —NHC(═O)CH₂NHC(═O)O—**, —CH₂N(X—R²)C(═O)O—**, or —C(═O)N(X—R²)—**, wherein each R_{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

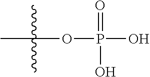 [SMILES: *OP(=O)(O)O]

groups;
A is a bond or —OC(═O)* in which * indicates the attachment point to D;
D is a Drug moiety as defined herein and comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.
   Embodiment 70. The immunoconjugate of Formula (E′) or any one of Embodiments 62 to 65, wherein
R¹⁰⁰ is

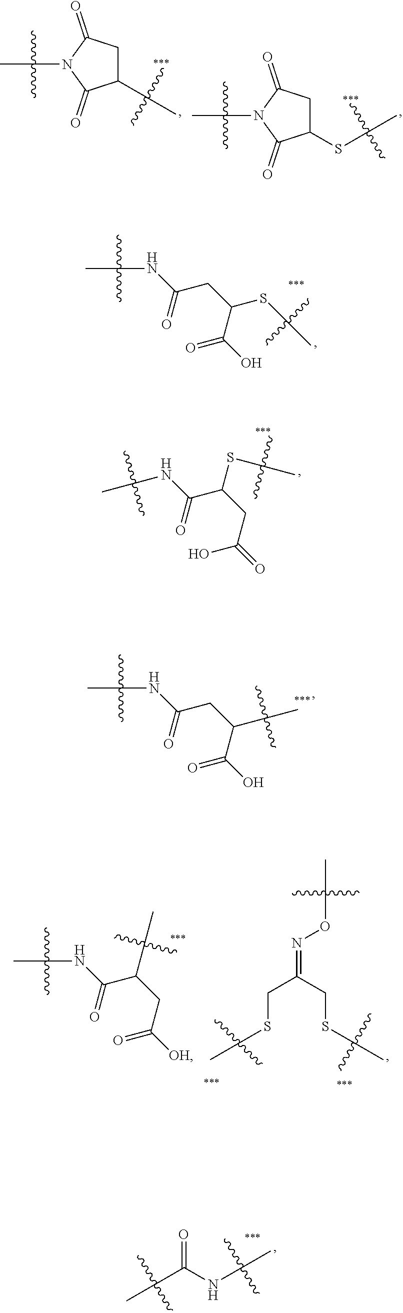

—S—, —C(═O)—, —ON═***, —NHC(═O)CH₂—***, —S(═O)₂CH₂CH₂—***, —(CH₂)₂S(═O)₂CH₂CH₂—***, —NHS(═O)₂CH₂CH₂—***, —NHC(═O)CH₂CH₂—***, —CH₂NHCH₂CH₂—***, —NHCH₂CH₂—***,

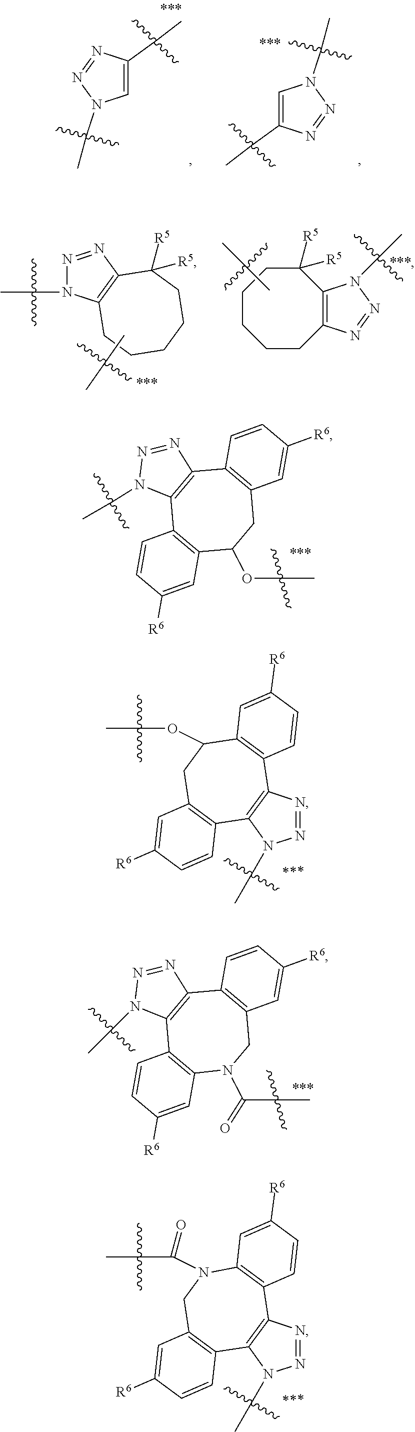

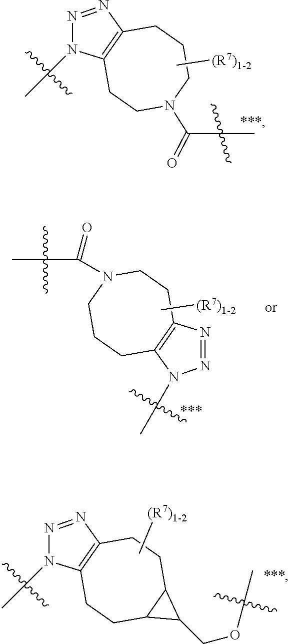

where the *** of R¹⁰⁰ indicates the point of attachment to Ab.
Embodiment 71. The immunoconjugate of Formula (E′) or any one of Embodiments 60 to 63, wherein
R¹⁰⁰ is

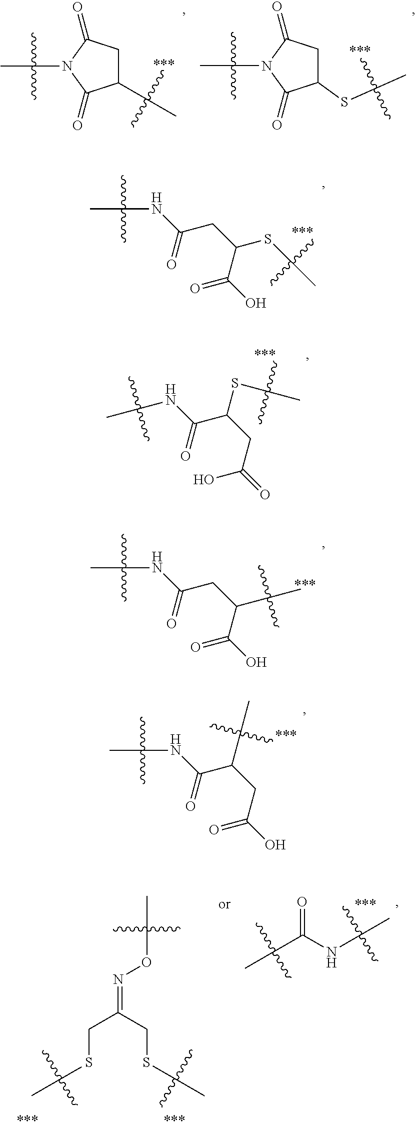

where the *** of R¹⁰⁰ indicates the point of attachment to Ab.
Embodiment 72. The immunoconjugate of Formula (E′) or any one of Embodiments 62 to 65, wherein
R¹⁰⁰ is

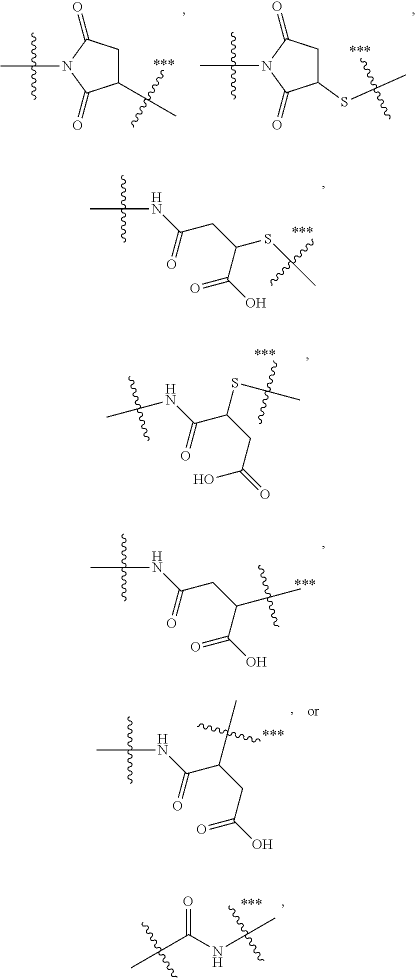

where the *** of R¹⁰⁰ indicates the point of attachment to Ab.
Embodiment 73. The immunoconjugate of Formula (E′) or any one of Embodiments 62 to 72 having the structure:

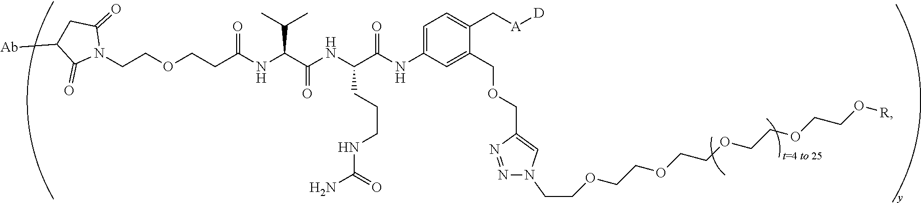

where R is H, —CH₃ or —CH₂CH₂C(═O)OH and y is 1, 2, 3, 4, 5, 6, 7, 8, 9, 10, 11, 12, 13, 14, 15 or 16.
Embodiment 74. The immunoconjugate of Formula (E′) or any one of Embodiments 62 to 72 having the structure:

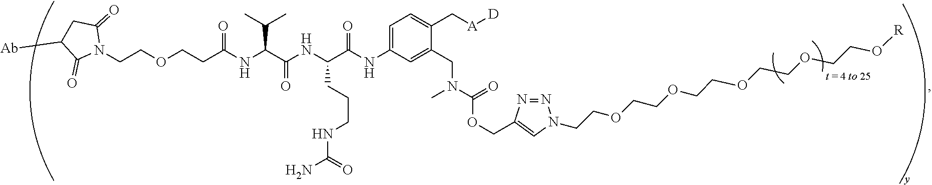

where
R is H, —CH₃ or —CH₂CH₂C(═O)OH and y is 1, 2, 3, 4, 5, 6, 7, 8, 9, 10, 11, 12, 13, 14, 15 or 16.
   Embodiment 75. The immunoconjugate of Formula (E′) or any one of Embodiments 62 to 72 having the structure:

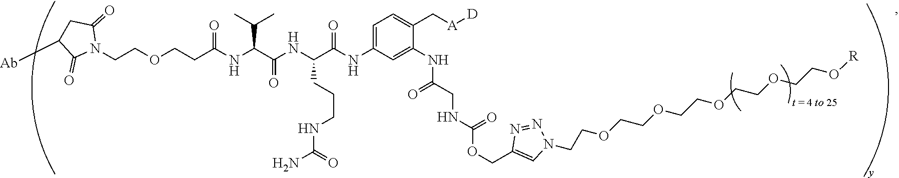

where R is H, —CH₃ or —CH₂CH₂C(═O)OH and y is 1, 2, 3, 4, 5, 6, 7, 8, 9, 10, 11, 12, 13, 14, 15 or 16.
Embodiment 76. The immunoconjugate of Formula (E′) or any one of Embodiments 62 to 72 having the structure:

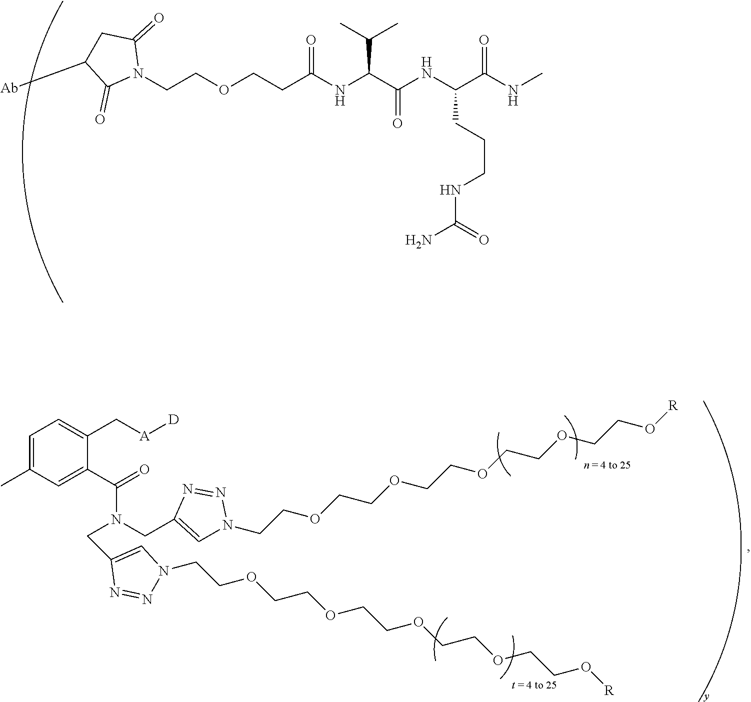

where
each R is independently selected from H, —CH₃ or —CH₂CH₂C(═O)OH and y is 1, 2, 3, 4, 5, 6, 7, 8, 9, 10, 11, 12, 13, 14, 15 or 16.
   Embodiment 77. The immunoconjugate of Formula (E′) or any one of Embodiments 62 to 72 having the structure:

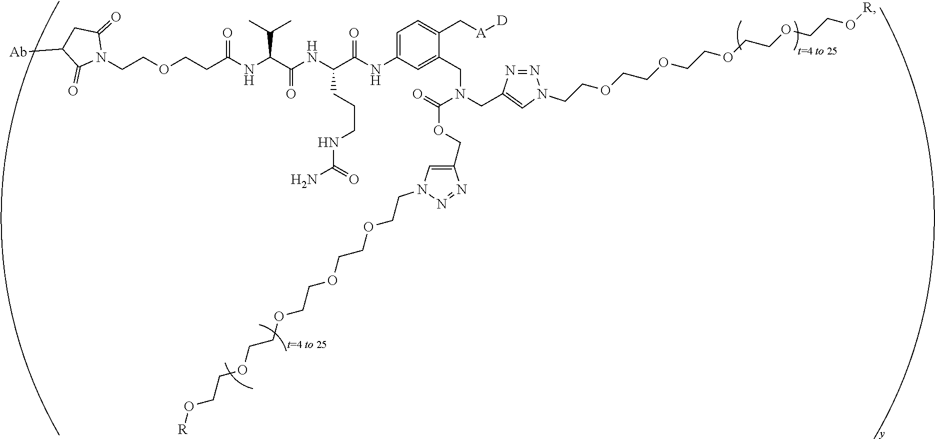

where
each R is independently selected from H, —CH₃ or —CH₂CH₂C(═O)OH and y is 1, 2, 3, 4, 5, 6, 7, 8, 9, 10, 11, 12, 13, 14, 15 or 16.
   Embodiment 78. The immunoconjugate of Formula (E′) or any one of Embodiments 62 to 72 having the structure:

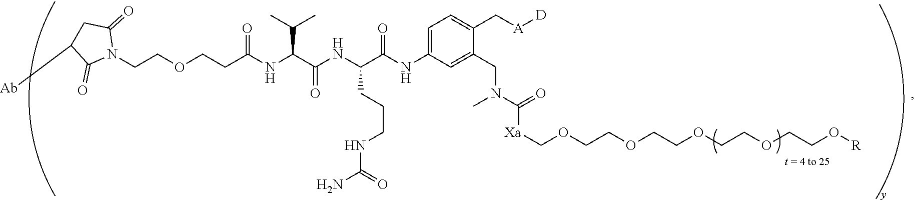

where
Xa is —CH₂—, —OCH₂—, —NHCH₂— or —NRCH₂— and each R is independently H, —CH₃ or —CH₂CH₂C(═O)OH and y is 1, 2, 3, 4, 5, 6, 7, 8, 9, 10, 11, 12, 13, 14, 15 or 16.
   Embodiment 79. The immunoconjugate of Formula (E′) or any one of Embodiments 62 to 72 having the structure:

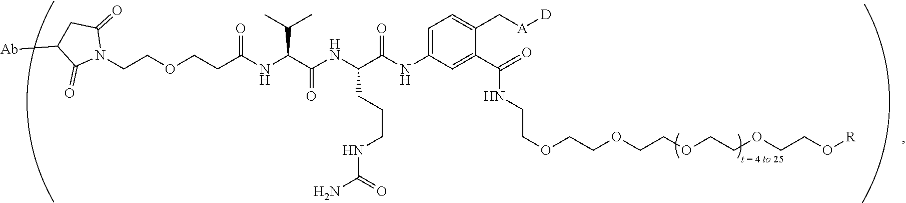

where R is H, —CH₃ or —CH₂CH₂C(═O)OH and y is 1, 2, 3, 4, 5, 6, 7, 8, 9, 10, 11, 12, 13, 14, 15 or 16.
Embodiment 80. The immunoconjugate of Formula (E′) or any one of Embodiments 62 to 72 having the structure:

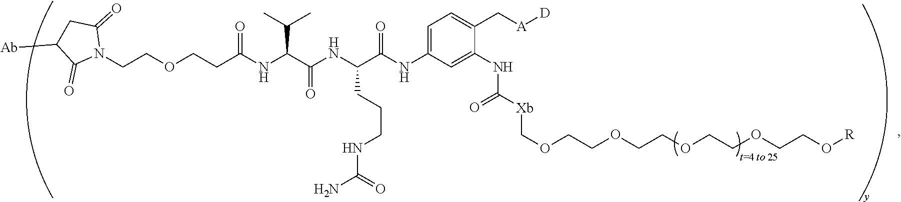

where Xb is —CH₂—, —OCH₂—, —NHCH₂— or —NRCH₂— and each R independently is H, —CH₃ or —CH₂CH₂C(═O)OH and y is 1, 2, 3, 4, 5, 6, 7, 8, 9, 10, 11, 12, 13, 14, 15 or 16.
Embodiment 81. The immunoconjugate of Formula (E′) or any one of Embodiments 62 to 72 having the structure:

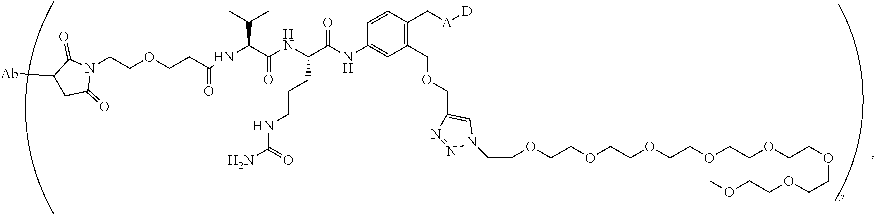

where y is 1, 2, 3, 4, 5, 6, 7, 8, 9, 10, 11, 12, 13, 14, 15 or 16.
Embodiment 82. The immunoconjugate of Formula (E′) or any one of Embodiments 62 to 72 having the structure:

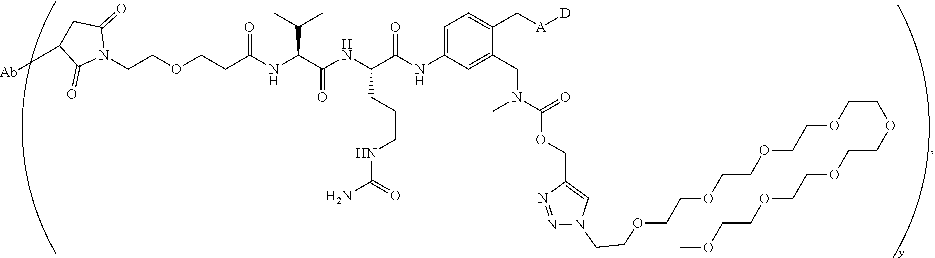

where y is 1, 2, 3, 4, 5, 6, 7, 8, 9, 10, 11, 12, 13, 14, 15 or 16.
Embodiment 83. The immunoconjugate of Formula (E′) or any one of Embodiments 62 to 72 having the structure:

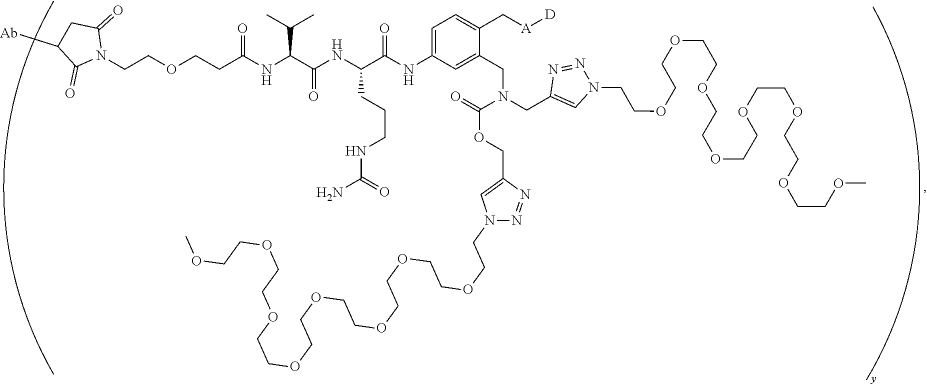

where y is 1, 2, 3, 4, 5, 6, 7, 8, 9, 10, 11, 12, 13, 14, 15 or 16.
Embodiment 84. The immunoconjugate of Formula (E′) or any one of Embodiments 62 to 72 having the structure:

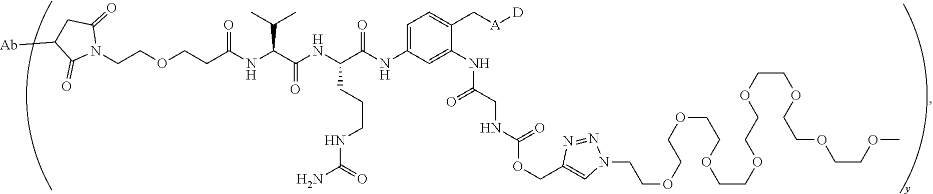

where y is 1, 2, 3, 4, 5, 6, 7, 8, 9, 10, 11, 12, 13, 14, 15 or 16.
Embodiment 85. The immunoconjugate of Formula (E′) or any one of Embodiments 62 to 72 having the structure:

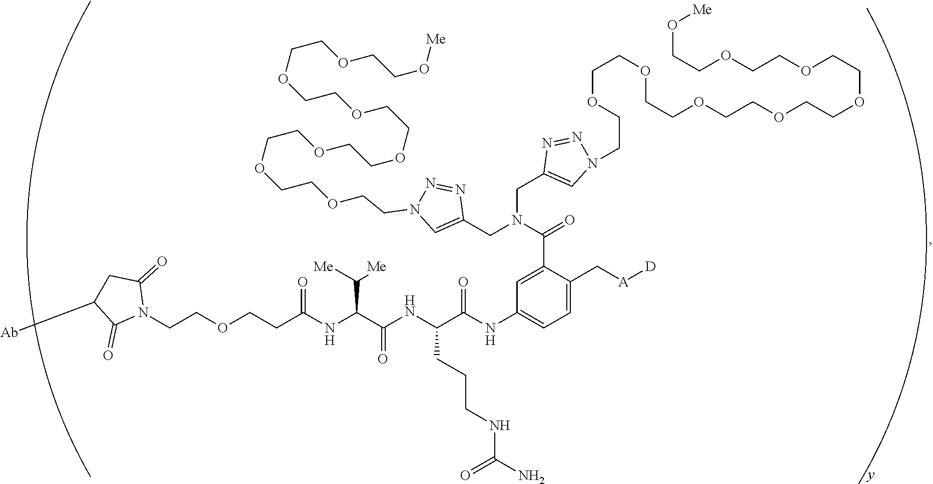

where y is 1, 2, 3, 4, 5, 6, 7, 8, 9, 10, 11, 12, 13, 14, 15 or 16
Embodiment 86. The immunoconjugate of Formula (E′) or any one of Embodiments 62 to 72 having the structure:

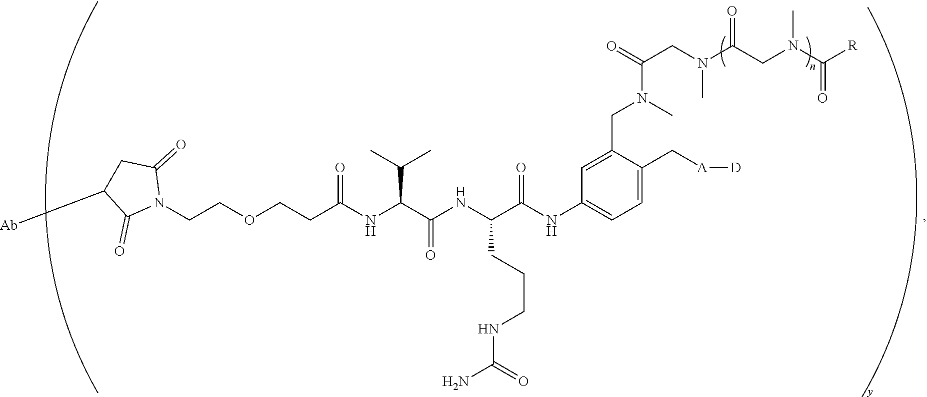

where y is 1, 2, 3, 4, 5, 6, 7, 8, 9, 10, 11, 12, 13, 14, 15 or 16.

Certain aspects and examples of the Linker-Drug groups, the Linkers and the Antibody Drug Conjugates of the invention are provided in the following listing of additional enumerated embodiments. It will be recognized that features specified in each embodiment may be combined with other specified features to provide further embodiments of the present invention.

Embodiment 87. The compound of Formula (A′) or any one of Embodiments 1 to 2, or pharmaceutically acceptable salt thereof, the linker of Formula (C′) or any one of Embodiments 33 to 40, and the immunoconjugate of Formula (E′) or any one of Embodiments 62 to 63, wherein:
G is

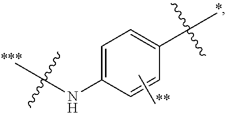

where the * of G indicates the point of attachment to L₂, and the ** of G indicates the point of attachment to L₃ and the *** of G indicates the point of attachment to Lp.
Embodiment 88. The compound of Formula (A′) or any one of Embodiments 1 to 2, or pharmaceutically acceptable salt thereof, the linker of Formula (C′) or any one of Embodiments 33 to 40, and the immunoconjugate of Formula (E′) or any one of Embodiments 62 to 63, wherein:
G is

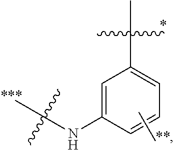

where the * of G indicates the point of attachment to L₂, and the ** of G indicates the point of attachment to L₃ and the *** of G indicates the point of attachment to Lp.
Embodiment 89. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R₃)₂(CH₂)ₘC(═O)NR₃(CH₂)ₘNR₃C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)(CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)(CH₂)ₘO)ₜ(CH₂)ₙNHC(═O)(CH₂)ₙX₁(CH₂)ₙ—*; *—C(═O)(CH₂)ₘO)ₜ(CH₂)ₙC(═O)NH(CH₂)ₘ—**; *—C(═O)(CH₂)ₘC(R₃)₂—** or *—C(═O)(CH₂)ₘC(═O)NH(CH₂)ₘ—**, where the * of L₁ indicates the point of attachment to Lp, and the ** of L₁ indicates the point of attachment to R¹ if present or the ** of L₁ indicates the point of attachment to R¹⁰⁰ if present.
   Embodiment 90. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, wherein:
L₁ is *—C(═O)(CH₂)ₘO(CH₂)ₘ—*; *—C(═O)((CH₂)ₘO)ₜ(CH₂)ₙ—**; *—C(═O)(CH₂)ₘ—*; *—C(═O)NH((CH₂)ₘO)ₜ(CH₂)ₙ—**; *—C(═O)(CH₂)ₘNH(CH₂)ₘ—**; *—C(═O)(CH₂)ₘNH(CH₂)ₙC(═O)—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)((CH₂)ₘO)ₜ(CH₂)ₙC(═O)NH(CH₂)ₘ—**; *—C(═O)(CH₂)ₘC(R₃)₂—** or *—C(═O)(CH₂)ₘC(═O)NH(CH₂)ₘ—**, where the * of L₁ indicates the point of attachment to Lp, and the ** of L₁ indicates the point of attachment to R¹ if present or the ** of L₁ indicates the point of attachment to R¹⁰⁰ if present.
   Embodiment 91. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)(CH₂)ₘNH(CH₂)ₘ—**; *—C(═O)(CH₂)ₘNH(CH₂)C(═O)—**; or *—C(═O)(CH₂)ₘNHC(═O)(CH₂)ₙ—*, where the * of L₁ indicates the point of attachment to Lp, and the ** of L₁ indicates the point of attachment to R¹ if present or the ** of L₁ indicates the point of attachment to R¹⁰⁰ if present.
   Embodiment 92. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—** or *—C(═O)NH((CH₂)ₘO)ₜ(CH₂)ₙ—**, where the * of L₁ indicates the point of attachment to Lp, and the ** of L₁ indicates the point of attachment to R¹ if present or the ** of L₁ indicates the point of attachment to R¹⁰⁰ if present.
   Embodiment 93. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, wherein L₁ is *—C(═O)(CH₂)ₘO(CH₂)ₘ—**, where the * of L₁ indicates the point of attachment to Lp, and the ** of L₁ indicates the point of attachment to R¹ if present or the ** of L₁ indicates the point of attachment to R¹⁰⁰ if present.
   Embodiment 94. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, wherein L₁ is *—C(═O)((CH₂)ₘO)ₜ(CH₂)ₙ—**, where the * of L₁ indicates the point of attachment to Lp, and the ** of L₁ indicates the point of attachment to R¹ if present or the ** of L₁ indicates the point of attachment to R¹⁰⁰ if present.
   Embodiment 95. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, wherein L₁ is *—C(═O)(CH₂)ₘ—**, where the * of L₁ indicates the point of attachment to Lp, and the ** of L₁ indicates the point of attachment to R¹ if present or the ** of L₁ indicates the point of attachment to R¹⁰⁰ if present.
   Embodiment 96. The compound of Formula (A′) or any one of Embodiments 1 to 17, or pharmaceutically acceptable salt thereof, the linker of Formula (C′) or any one of Embodiments 32 to 46, and the immunoconjugate of Formula (E′) or any one of Embodiments 60 to 70, wherein L₁ is *—C(═O)NH((CH₂)ₘO)ₜ(CH₂)ₙ—**, where the * of L₁ indicates the point of attachment to Lp, and the ** of L₁ indicates the point of attachment to R¹ if present or the ** of L₁ indicates the point of attachment to R¹⁰⁰ if present.
   Embodiment 97. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 84 to 93, wherein Lp is an enzymatically cleavable bivalent peptide spacer.
   Embodiment 98. The compound of Formula (A′) or any one of Embodiments 1 to 17, or pharmaceutically acceptable salt thereof, the linker of Formula (C′) or any one of Embodiments 32 to 46, and the immunoconjugate of Formula (E′) or any one of Embodiments 60 to 70, or any one of Embodiments 87 to 97, wherein Lp is a bivalent peptide spacer comprising an amino acid residue selected from glycine, valine, citrulline, lysine, isoleucine, phenylalanine, methionine, asparagine, proline, alanine, leucine, tryptophan, and tyrosine.
   Embodiment 99. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 98, wherein Lp is a bivalent peptide spacer comprising two to four amino acid residues.
   Embodiment 100. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 99, wherein Lp is a bivalent peptide spacer comprising two to four amino acid residues each independently selected from glycine, valine, citrulline, lysine, isoleucine, phenylalanine, methionine, asparagine, proline, alanine, leucine, tryptophan, and tyrosine.
   Embodiment 101. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 100, wherein:
Lp is a bivalent peptide spacer selected from

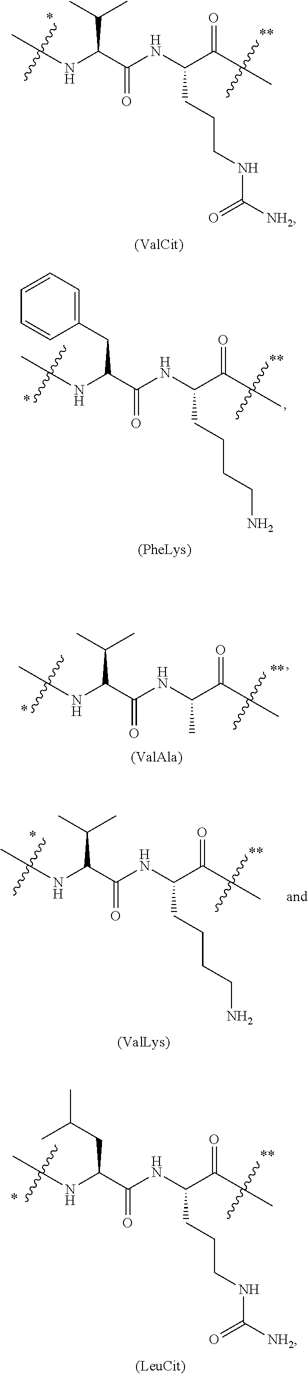

where the * of Lp indicates the attachment point to L₁ and the ** of Lp indicates the attachment point to the —NH— group of Formula (B′) or the ** of Lp indicates the attachment point to the G of Formula (A′).
Embodiment 102. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 101, wherein:
Lp is

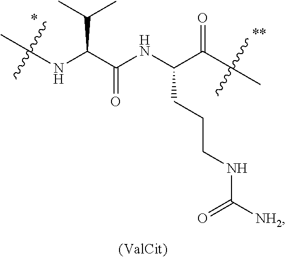 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of Formula (B′) or the ** of Lp indicates the attachment point to the G of Formula (A′).
Embodiment 103. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 101, wherein:
Lp is

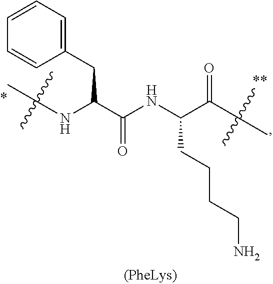

where the * of Lp indicates the attachment point to L₁ and the ** of Lp indicates the attachment point to the —NH— group of Formula (B′) or the ** of Lp indicates the attachment point to the G of Formula (A′).
Embodiment 104. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 101, wherein:
Lp is

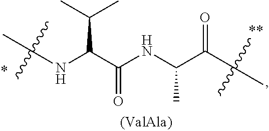

where the * of Lp indicates the attachment point to L₁ and the ** of Lp indicates the attachment point to the —NH— group of Formula (B′) or the ** of Lp indicates the attachment point to the G of Formula (A′).
Embodiment 105. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 101, wherein:
Lp is

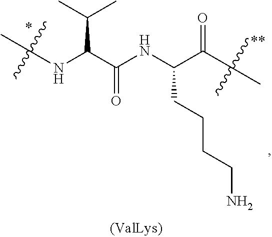

where the * of Lp indicates the attachment point to L₁ and the ** of Lp indicates the attachment point to the —NH— group of Formula (B′) or the ** of Lp indicates the attachment point to the G of Formula (A′).
Embodiment 106. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 101, wherein:
Lp is

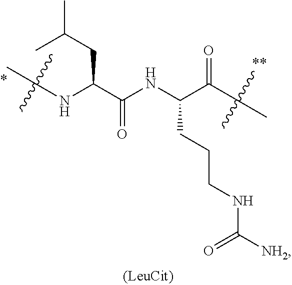 [SMILES: *N[C@@H](CC(C)C)C(=O)N[C@@H](CCCNC(N)=O)C(*)=O]

where the * of Lp indicates the attachment point to L₁ and the ** of Lp indicates the attachment point to —NH— group of Formula (B′) or the ** of Lp indicates the attachment point to the G of Formula (A′).
Embodiment 107. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 106, wherein L₂ is a bond, a methylene, or a C₂-C₃alkenylene.
Embodiment 108. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 107, wherein L₂ is a bond or a methylene.
Embodiment 109. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 108, wherein L₂ is a bond.
Embodiment 110. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 108, wherein L₂ is a methylene.
Embodiment 111. The compound of Formula (A′) or any one of Embodiments 1 to 30, or pharmaceutically acceptable salt thereof, the linker of Formula (C′) or any one of Embodiments 33 to 47, and the immunoconjugate of Formula (E′) or any one of Embodiments 62 to 85, or any one of Embodiments 87 to 110, wherein:
A is a bond, —OC(═O)—, —OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl.
   Embodiment 112. The compound of Formula (A′) or any one of Embodiments 1 to 32, or pharmaceutically acceptable salt thereof, the linker of Formula (C′) or any one of Embodiments 33 to 61, and the immunoconjugate of Formula (E′) or any one of Embodiments 62 to 86, or any one of Embodiments 87 to 111, wherein A is a bond or —OC(═O).
   Embodiment 113. The compound of Formula (A′) or any one of Embodiments 1 to 32, or pharmaceutically acceptable salt thereof, the linker of Formula (C′) or any one of Embodiments 33 to 61, and the immunoconjugate of Formula (E′) or any one of Embodiments 62 to 86, or any one of Embodiments 87 to 112, wherein A is a bond.
   Embodiment 114. The compound of Formula (A′) or any one of Embodiments 1 to 32, or pharmaceutically acceptable salt thereof, the linker of Formula (C′) or any one of Embodiments 33 to 61, and the immunoconjugate of Formula (E′) or any one of Embodiments 62 to 86, or any one of Embodiments 87 to 112, wherein A is —OC(═O).
   Embodiment 115. The compound of Formula (A′) or any one of Embodiments 1 to 32, or pharmaceutically acceptable salt thereof, the linker of Formula (C′) or any one of Embodiments 33 to 61, and the immunoconjugate of Formula (E′) or any one of Embodiments 62 to 86, or any one of Embodiments 87 to 110, wherein:
A is

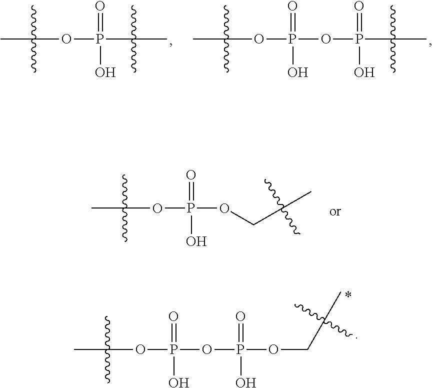 [SMILES: *OP(*)(=O)O ; *OP(=O)(O)OP(*)(=O)O ; *COP(=O)(O)O* ; *COP(=O)(O)OP(=O)(O)O*]

Embodiment 116. The compound of Formula (A′) or any one of Embodiments 1 to 32, or pharmaceutically acceptable salt thereof, the linker of Formula (C′) or any one of Embodiments 33 to 61, and the immunoconjugate of Formula (E′) or any one of Embodiments 62 to 85, or any one of Embodiments 86 to 110, wherein:
A is —OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈cycloalkyl.
   Embodiment 117. The compound of Formula (A′) or any one of Embodiments 1 to 17, or pharmaceutically acceptable salt thereof, the linker of Formula (C′) or any one of Embodiments 33 to 49, and the immunoconjugate of Formula (E′) or any one of Embodiments 62 to 72, or any one of Embodiments 87 to 116, wherein:
L₃ is a spacer moiety having the structure

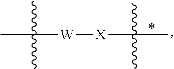

where
   W is —CH₂O—**, —CH₂N(R^{b})C(═O)O—**, —NHC(═O)C(R^{b})₂NHC(═O)O—**, —NHC(═O)C(R^{b})₂NH—**, —NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
the * of L₃ indicates the point of attachment to R².
   Embodiment 118. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 117, wherein:
L₃ is a spacer moiety having the structure

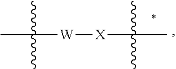

where
   W is —CH₂O—**, —CH₂N(R^{b})C(═O)O—**, —NHC(═O)CH₂NHC(═O)O—**, —NHC(═O)CH₂NH—**, —NHC(═O)CH₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond;
   and
the * of L₃ indicates the point of attachment to R².
   Embodiment 119. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 118, wherein:
L₃ is a spacer moiety having the structure

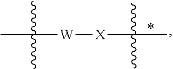

where
   W is —CH₂O—**, —CH₂N(R^{b})C(═O)O—**, —NHC(═O)CH₂NHC(═O)O—**, —NHC(═O)CH₂NH—**, —NHC(═O)CH₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a triazolyl, wherein the *** of X indicates the point of attachment to W and the * of X indicates the point of attachment to R²;
   and
the * of L₃ indicates the point of attachment to R².
   Embodiment 120. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 118, wherein:
L₃ is a spacer moiety having the structure

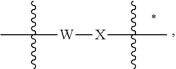

where
   W is —CH₂O—**, —CH₂N(R^{b})C(═O)O—**, —NHC(═O)CH₂NHC(═O)O—**, —NHC(═O)CH₂NH—**, —NHC(═O)CH₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NRDC(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
X is ***—CH₂-triazolyl-*, wherein the *** of X indicates the point of attachment to W and the * of X indicates the point of attachment to R²;
and the * of L₃ indicates the point of attachment to R².
   Embodiment 121. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 118, wherein:
L₃ is a spacer moiety having the structure

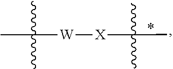

where
   W is —CH₂O—**, —CH₂N(R^{b})C(═O)O—**, —NHC(═O)CH₂NHC(═O)O—**, —CH₂N(X—R₂)C(═O)O—**, —C(═O)N(X—R²)—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
the * of L₃ indicates the point of attachment to R².
   Embodiment 122. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 118, wherein:
L₃ is a spacer moiety having the structure

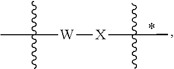

where
   W is —CH₂O—**, —CH₂N(R^{b})C(═O)O—**, —NHC(═O)CH₂NHC(═O)O—**, —CH₂N(X—R₂)C(═O)O—**, —C(═O)N(X—R²)—**, wherein each R^{b} is independently selected from H, C₁-C₆alkyl or C₃-C₈cycloalkyl and wherein the ** of W indicates the point of attachment to X;
   X is a bond;
   and
the * of L₃ indicates the point of attachment to R².
   Embodiment 123. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 83 to 118, wherein:
L₃ is a spacer moiety having the structure

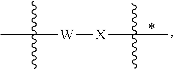

where
   W is —CH₂O—**, —CH₂N(R^{b})C(═O)O—**, —NHC(═O)CH₂NHC(═O)O—**, —CH₂N(X—R₂)C(═O)O—**, —C(═O)N(X—R²)—**, wherein each R^{b} is independently selected from H, C₁-C₆alkyl or C₃-C₈cycloalkyl and wherein the ** of W indicates the point of attachment to X;
   X is a triazolyl, wherein the *** of X indicates the point of attachment to W and the * of X indicates the point of attachment to R²;
   and
the * of L₃ indicates the point of attachment to R².
   Embodiment 124. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 118, wherein:
L₃ is a spacer moiety having the structure

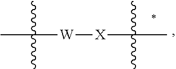

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²;
   and
the * of L₃ indicates the point of attachment to R².
   Embodiment 125. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 86 to 124, wherein R² is a hydrophilic moiety selected from polyethylene glycol, polyalkylene glycol, a sugar, an oligosaccharide, a polypeptide or C₂-C₆alkyl substituted with 1 to 3

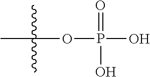 [SMILES: *OP(=O)(O)O]

groups.
Embodiment 126. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein R² is a sugar.
Embodiment 127. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein R² is an oligosaccharide.
Embodiment 128. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein R² is a polypeptide.
Embodiment 129. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein R² is a polyalkylene glycol.
Embodiment 130. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein R² is a polyalkylene glycol having the structure —(O(CH₂)ₘ)ₜR′, where R′ is OH, OCH₃ or OCH₂CH₂C(═O)OH, m is 1-10 and t is 4-40.
Embodiment 131. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein R² is a polyalkylene glycol having the structure —(CH₂)ₘO)ₜR″—, where R″ is H, CH₃ or CH₂CH₂C(═O)OH, m is 1-10 and t is 4-40.
Embodiment 132. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein R² is a polyethylene glycol.
Embodiment 133. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein R² is a polyethylene glycol having the structure —(OCH₂CH₂)ₜR′, where R′ is OH, OCH₃ or OCH₂CH₂C(═O)OH and t is 4-40,
Embodiment 134. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein R² is a polyethylene glycol having the structure —(CH₂CH₂O)ₜR″—, where R″ is H, CH₃ or CH₂CH₂C(═O)OH and t is 4-40.
Embodiment 135. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein:
R² is

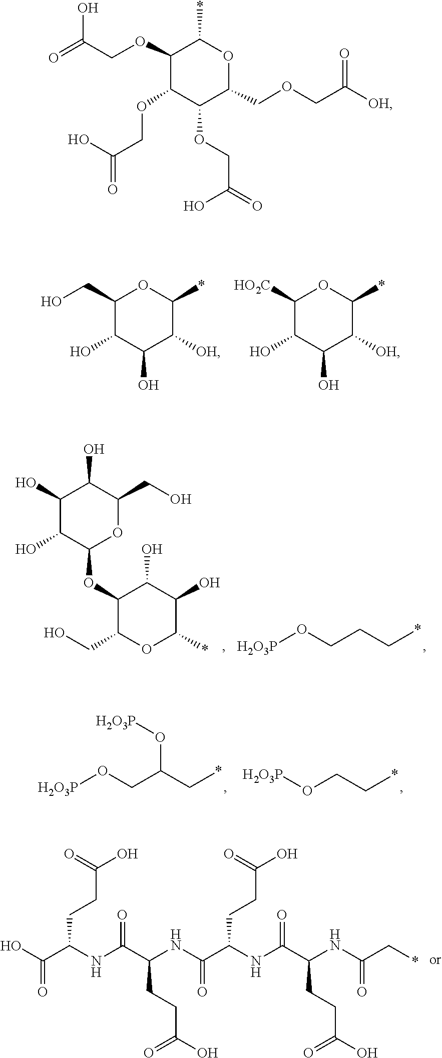

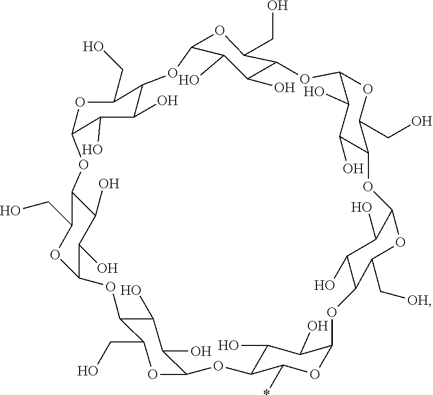 [SMILES: *C1OC2OC3C(CO)OC(OC4C(CO)OC(OC5C(CO)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O]

where the * of R² indicates the point of attachment to X or L₃.
Embodiment 136. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein:
R² is

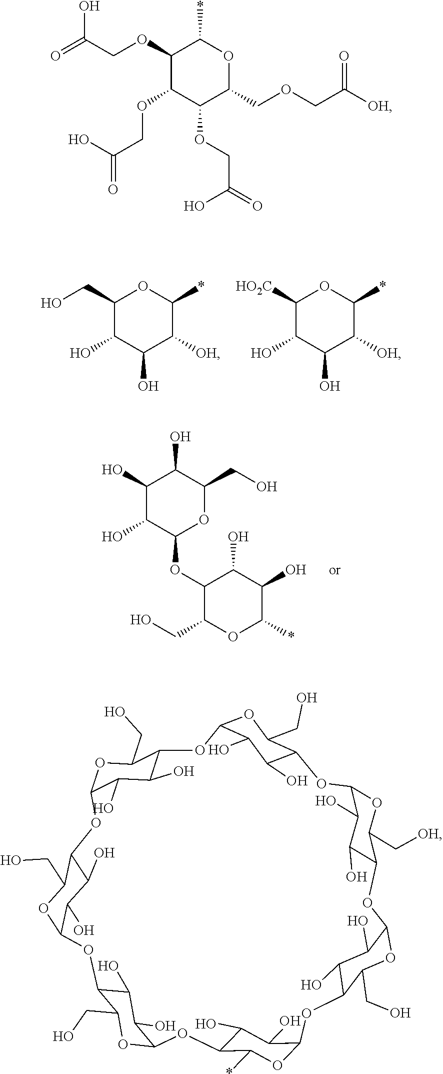 [SMILES: *[C@@H]1O[C@H](COCC(=O)O)[C@H](OCC(=O)O)[C@H](OCC(=O)O)[C@H]1OCC(=O)O ; *[C@@H]1O[C@H](CO)[C@@H](O)[C@H](O)[C@H]1O ; *[C@@H]1O[C@H](C(=O)O)[C@@H](O)[C@H](O)[C@H]1O ; *[C@@H]1O[C@H](CO)C(O[C@@H]2O[C@H](CO)[C@H](O)[C@H](O)[C@H]2O)[C@H](O)[C@H]1O ; *C1OC2OC3C(CO)OC(OC4C(CO)OC(OC5C(CO)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O]

where the * of R² indicates the point of attachment to X or L₃.
Embodiment 137. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein:
R² is

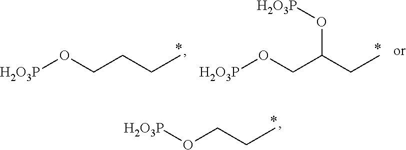

where the * of R² indicates the point of attachment to X or L₃.
Embodiment 138. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 125, wherein:
R² is

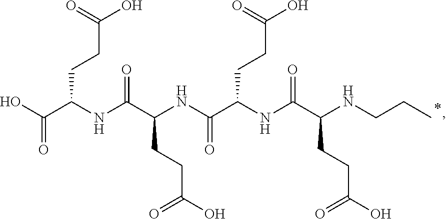

where the * of R² indicates the point of attachment to X or L₃.
Embodiment 139. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 138, wherein:
X₁ is

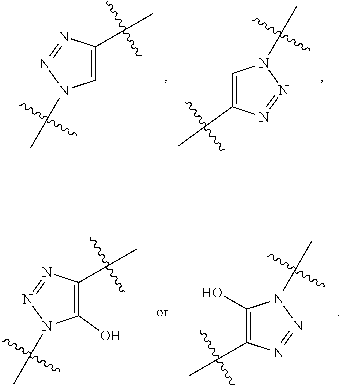 [SMILES: *c1cn(*)nn1 ; *c1cn(*)nn1 ; *c1nnn(*)c1O ; *c1nnn(*)c1O]

Embodiment 140. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 138, wherein:
X₁ is

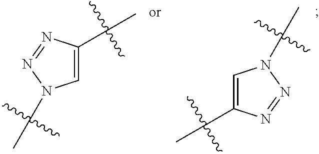 [SMILES: *c1cn(*)nn1 ; *c1cn(*)nn1]

Embodiment 141. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 140, wherein:
each m is independently selected from 1, 2, 3, 4, and 5.
   Embodiment 142. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 140, wherein:
each m is independently selected from 1, 2 and 3.
   Embodiment 143. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 142, wherein:
each n is independently selected from 1, 2, 3, 4 and 5.
   Embodiment 144. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 142, wherein:
each n is independently selected from 1, 2 and 3.
   Embodiment 145. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 144, wherein:
each t is independently selected from 2, 3, 4, 5, 6, 7, 8, 9, 10, 11, 12, 13, 14, 15, 16, 17, 18, 19, 20, 21, 22, 23, 24, 25, 26, 27, 28, 29 and 30.
   Embodiment 146. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 144, wherein:
each t is independently selected from 4, 5, 6, 7, 8, 9, 10, 11, 12, 13, 14, 15, 16, 17, 18, 19, 20, 21, 22, 23, 24 or 25.
   Embodiment 147. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 144, wherein:
each t is independently selected from 1, 2, 3, 4, 5, 6, 7, 8, 9, 10, 11, 12, 13, 14, 15, 16, 17 and 18.
   Embodiment 148. The immunoconjugate of Formula (E′) or any one of Embodiments 62 to 72, or any one of Embodiments 87 to 147, wherein y is 1, 2, 3, 4, 5, 6, 7, 8, 9, 10, 11, 12, 13, or 14.
   Embodiment 149. The immunoconjugate of Formula (E′) or any one of Embodiments 62 to 72, or any one of Embodiments 87 to 147, wherein y is 1, 2, 3, 4, 5, 6, 7, 8, 9, 10, 11 or 12.
   Embodiment 150. The immunoconjugate of Formula (E′) or any one of Embodiments 62 to 72, or any one of Embodiments 87 to 147, wherein y is 1, 2, 3, 4, 5, 6, 7, 8, 9 or 10.
   Embodiment 151. The immunoconjugate of Formula (E′) or any one of Embodiments 62 to 72, or any one of Embodiments 87 to 147, wherein y is 1, 2, 3, 4, 5, 6, 7 or 8.
   Embodiment 152. The immunoconjugate of Formula (E′) or any one of Embodiments 62 to 72, or any one of Embodiments 87 to 147, wherein y is 1, 2, 3, 4, 5 or 6.
   Embodiment 153. The immunoconjugate of Formula (E′) or any one of Embodiments 62 to 72, or any one of Embodiments 87 to 147, wherein y is 1, 2, 3 or 4.
   Embodiment 154. The immunoconjugate of Formula (E′) or any one of Embodiments 62 to 72, or any one of Embodiments 87 to 147, wherein y is 1 or 2.
   Embodiment 155. The immunoconjugate of Formula (E′) or any one of Embodiments 62 to 72, or any one of Embodiments 87 to 147, wherein y is 2.
   Embodiment 156. The immunoconjugate of Formula (E′) or any one of Embodiments 62 to 72, or any one of Embodiments 87 to 147, wherein y is 4.
   Embodiment 157. The immunoconjugate of Formula (E′) or any one of Embodiments 62 to 72, or any one of Embodiments 87 to 147, wherein y is 6.
   Embodiment 158. The immunoconjugate of Formula (E′) or any one of Embodiments 62 to 72, or any one of Embodiments 87 to 147, wherein y is 8.
   Embodiment 159. The compound of Formula (A′) or any one of Embodiments 1 to 31, or pharmaceutically acceptable salt thereof, the immunoconjugate of Formula (E′) or any one of Embodiments 62 to 72, or any one of Embodiments 87 to 158, wherein D is a Bcl-xL inhibitor when released from the immunoconjugates.

### Other Linker Groups

Other examples of linker groups that are suitable for making ADCs or immunoconjugates of a Bcl-xL inhibitor disclosed herein includes those disclosed in international application publications such as WO2018200812, WO2017214456, WO2017214458, WO2017214462, WO2017214233, WO2017214282, WO2017214301, WO2017214322, WO2017214335, WO2017214339, WO2016094509, WO2016094517, and WO2016094505, the contents of each of which are incorporated by reference in their entireties.

For example, the immunoconjugates of Bcl-xL inhibitors disclosed herein can have a linker-payload (“-L-D”) structure selected from:

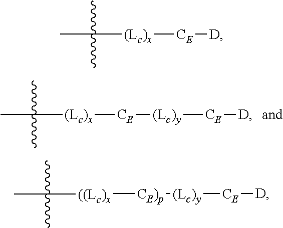

wherein:
Lc is a linker component and each Lc is independently selected from a linker component as disclosed herein;
x is an integer selected from 1, 2, 3, 4, 5, 6, 7, 8, 9, 10, 11, 12, 13, 14, 15, 16, 17, 18, 19 and 20;
y is an integer selected from 1, 2, 3, 4, 5, 6, 7, 8, 9, 10, 11, 12, 13, 14, 15, 16, 17, 18, 19 and 20;
p is an integer selected from 1, 2, 3, 4, 5, 6, 7, 8, 9 and 10;
D is a Bcl-xL inhibitor disclosed herein;
and each cleavage element (C_{E}) is independently selected from a self-immolative spacer and a group that is susceptible to cleavage selected from acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage.

In some embodiments, L has a structure selected from the following, or L comprises a structural component selected from the following:

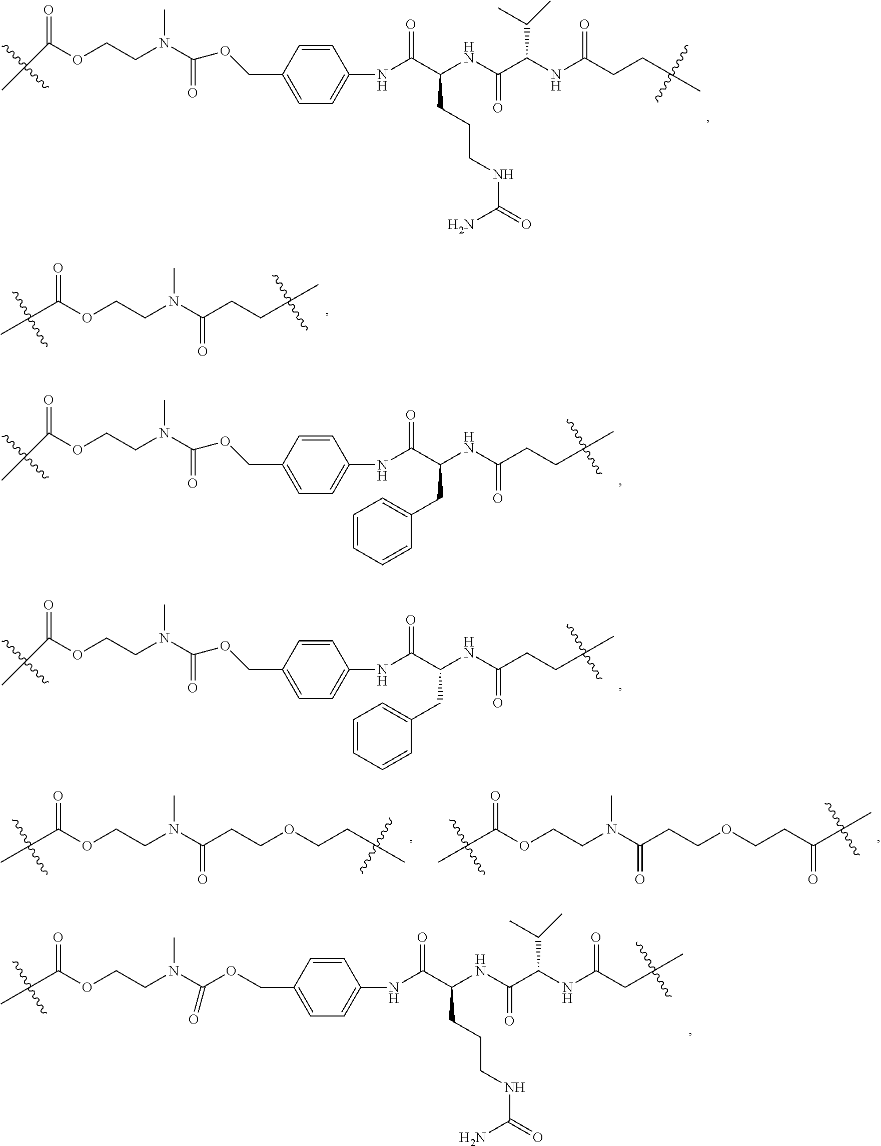

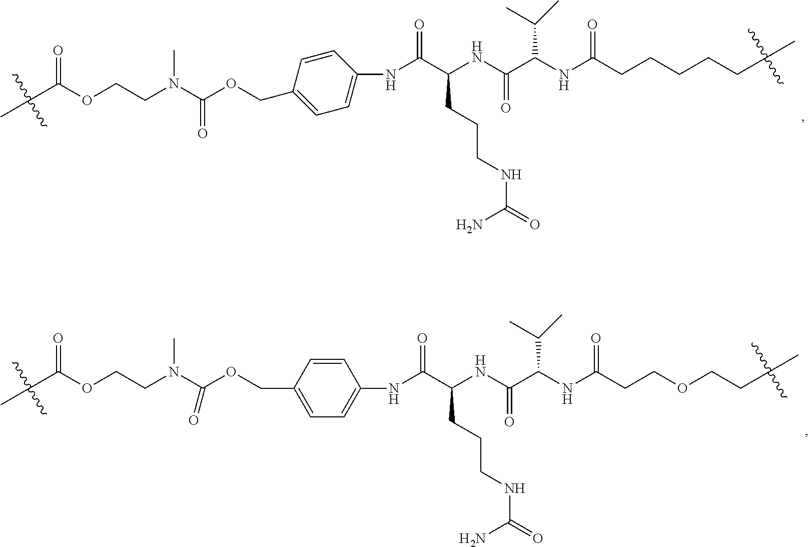

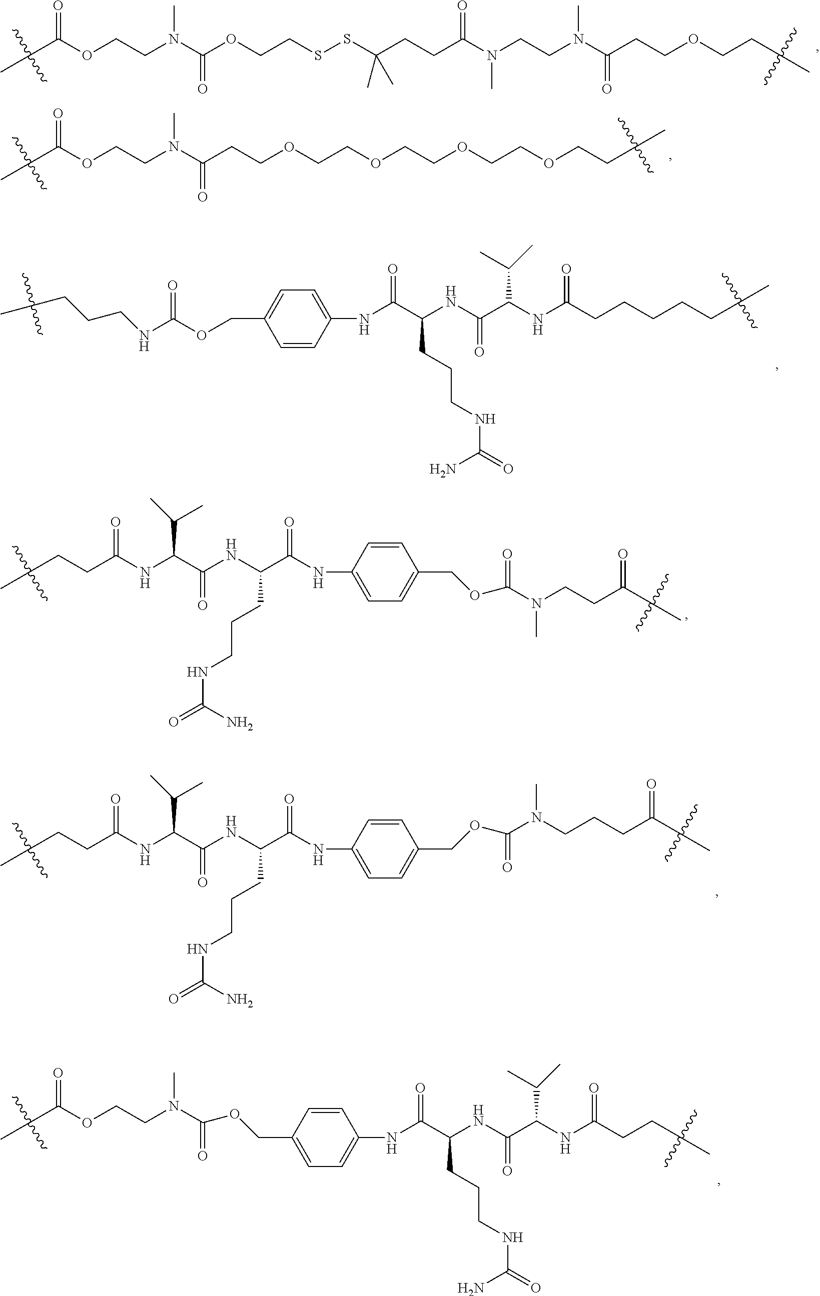

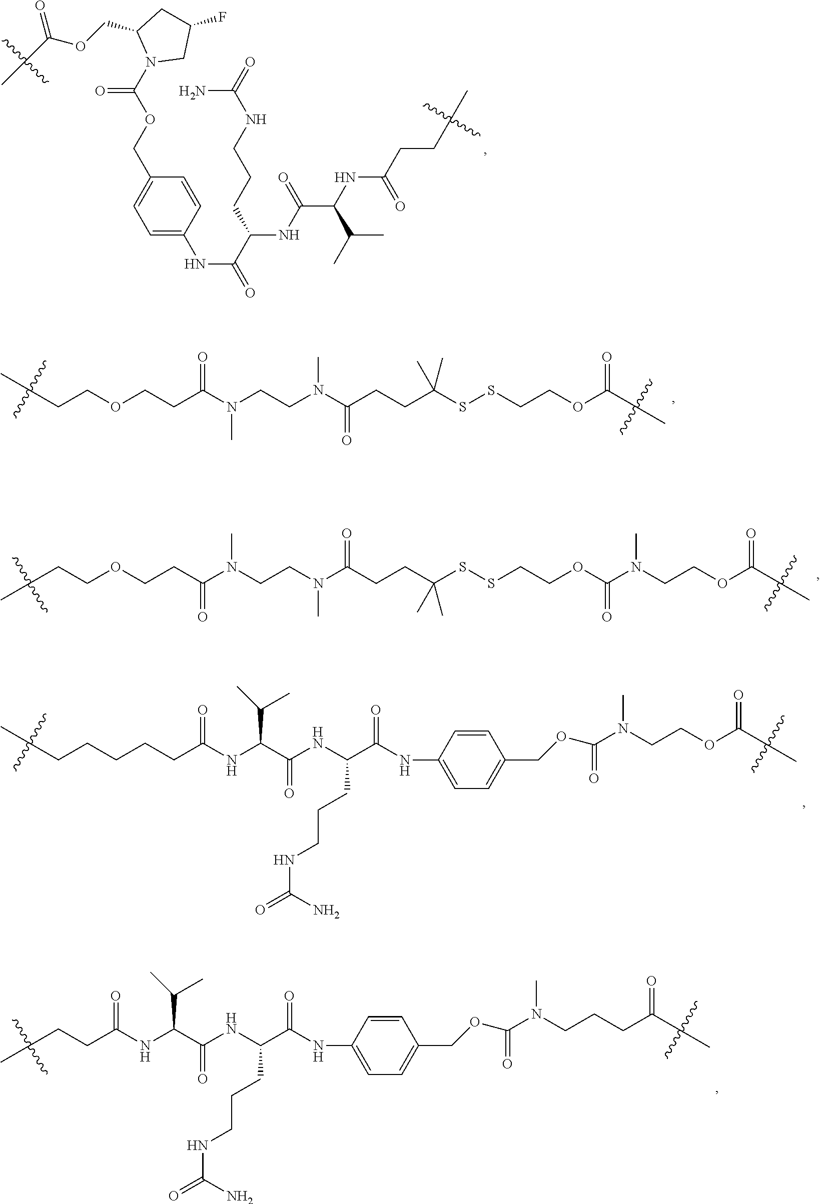 [SMILES: *CCC(=O)N[C@H](C(=O)N[C@@H](CCCNC(N)=O)C(=O)Nc1ccc(COC(=O)N2C[C@@H](F)C[C@H]2COC(*)=O)cc1)C(C)C ; *CCOCCC(=O)N(C)CCN(C)C(=O)CCC(C)(C)SSCCOC(*)=O ; *CCOCCC(=O)N(C)CCN(C)C(=O)CCC(C)(C)SSCCOC(=O)N(C)CCOC(*)=O ; *CCCCCC(=O)N[C@H](C(=O)N[C@@H](CCCNC(N)=O)C(=O)Nc1ccc(COC(=O)N(C)CCOC(*)=O)cc1)C(C)C ; *CCC(=O)N[C@H](C(=O)N[C@@H](CCCNC(N)=O)C(=O)Nc1ccc(COC(=O)N(C)CCCC(*)=O)cc1)C(C)C]

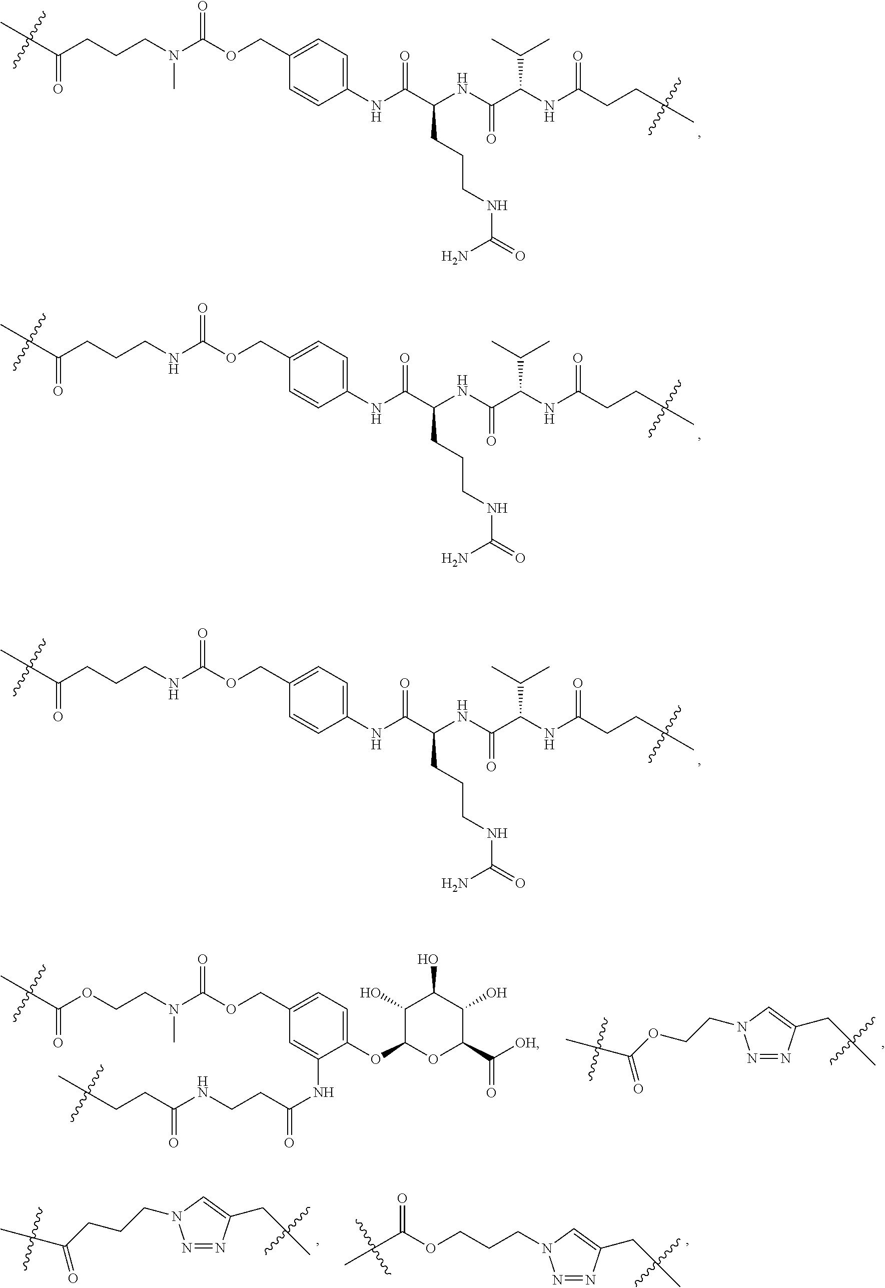

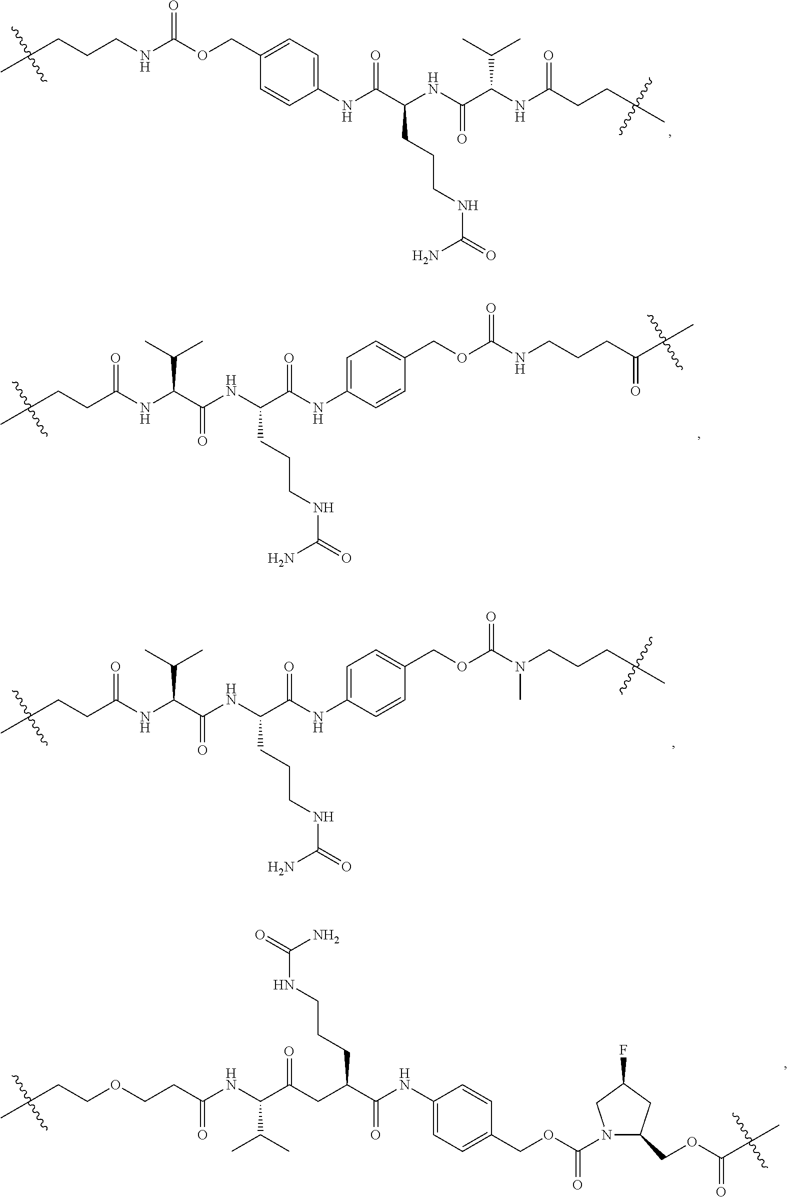 [SMILES: *CCCNC(=O)OCc1ccc(NC(=O)[C@H](CCCNC(N)=O)NC(=O)[C@@H](NC(=O)CC*)C(C)C)cc1 ; *CCC(=O)N[C@H](C(=O)N[C@@H](CCCNC(N)=O)C(=O)Nc1ccc(COC(=O)NCCCC(*)=O)cc1)C(C)C ; *CCCN(C)C(=O)OCc1ccc(NC(=O)[C@H](CCCNC(N)=O)NC(=O)[C@@H](NC(=O)CC*)C(C)C)cc1 ; *CCOCCC(=O)N[C@H](C(=O)C[C@@H](CCCNC(N)=O)C(=O)Nc1ccc(COC(=O)N2C[C@@H](F)C[C@H]2COC(*)=O)cc1)C(C)C]

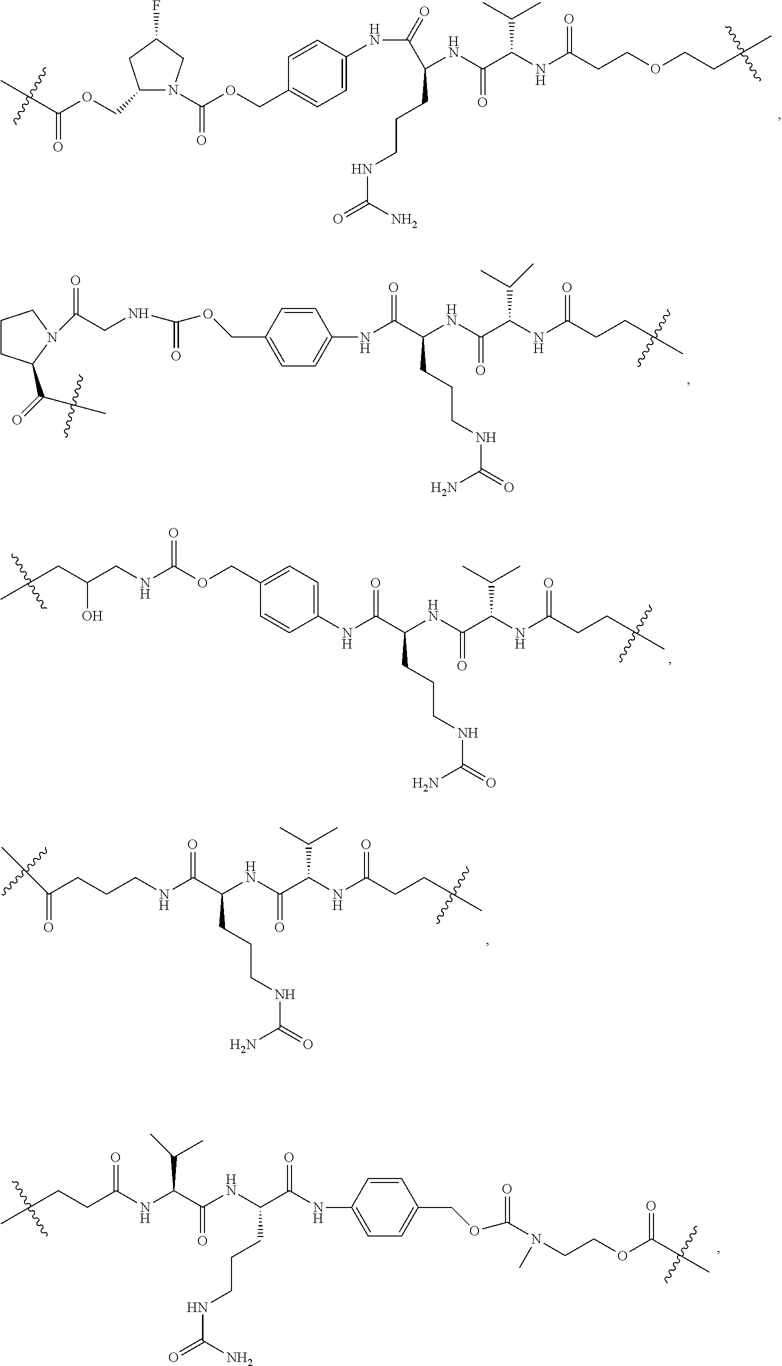 [SMILES: *CCOCCC(=O)N[C@H](C(=O)N[C@@H](CCCNC(N)=O)C(=O)Nc1ccc(COC(=O)N2C[C@@H](F)C[C@H]2COC(*)=O)cc1)C(C)C ; *CCC(=O)N[C@H](C(=O)N[C@@H](CCCNC(N)=O)C(=O)Nc1ccc(COC(=O)NCC(=O)N2CCC[C@@H]2C(*)=O)cc1)C(C)C ; *CCC(=O)N[C@H](C(=O)N[C@@H](CCCNC(N)=O)C(=O)Nc1ccc(COC(=O)NCC(O)C*)cc1)C(C)C ; *CCC(=O)N[C@H](C(=O)N[C@@H](CCCNC(N)=O)C(=O)NCCCC(*)=O)C(C)C ; *CCC(=O)N[C@H](C(=O)N[C@@H](CCCNC(N)=O)C(=O)Nc1ccc(COC(=O)N(C)CCOC(*)=O)cc1)C(C)C]

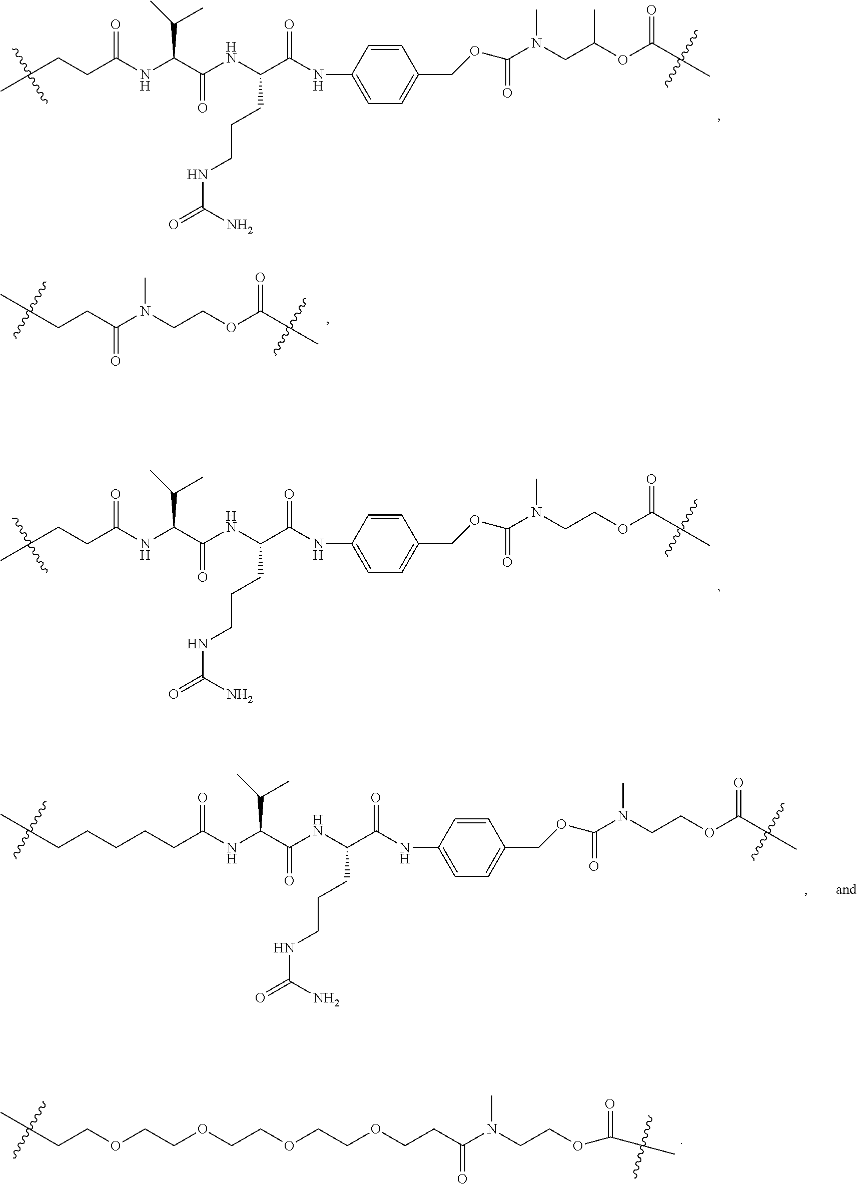 [SMILES: *CCC(=O)N[C@H](C(=O)N[C@@H](CCCNC(N)=O)C(=O)Nc1ccc(COC(=O)N(C)CC(C)OC(*)=O)cc1)C(C)C ; *CCC(=O)N(C)CCOC(*)=O ; *CCC(=O)N[C@H](C(=O)N[C@@H](CCCNC(N)=O)C(=O)Nc1ccc(COC(=O)N(C)CCOC(*)=O)cc1)C(C)C ; *CCCCCC(=O)N[C@H](C(=O)N[C@@H](CCCNC(N)=O)C(=O)Nc1ccc(COC(=O)N(C)CCOC(*)=O)cc1)C(C)C ; *CCOCCOCCOCCOCCC(=O)N(C)CCOC(*)=O]

In some embodiments, Lc is a linker component and each Lc is independently selected from

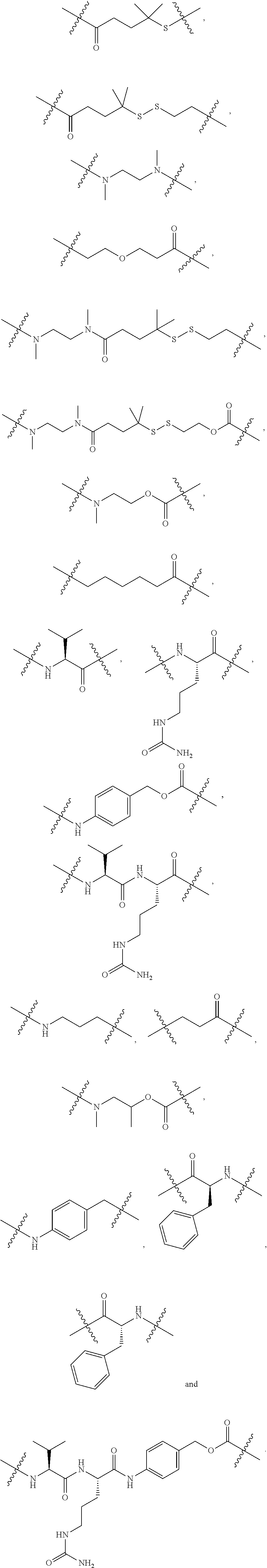

In some embodiments, the linker L comprises a linker component that is selected from: —**C(═O)O(CH₂)ₘNR¹¹C(═O)(CH₂)ₘ—; —**C(═O)O(CH₂)NR¹¹C(═O)(CH₂)ₘO(CH₂)ₘ—; —**C(═O)O(CH₂)ₘNR¹¹C(═O)X₁ₐX₂ₐC(═O)(CH₂)ₘ—; —**C(═O)OC(R¹²)₂(CH₂)ₘNR¹¹C(═O)X₁ₐX₂ₐC(═O)(CH₂)ₘ—; —**C(═O)O(CH₂)ₘNR¹¹C(═O)X₁ₐX₂ₐC(═O)(CH₂)ₘO(CH₂)ₘ—; —**C(═O)O(CH₂)ₘNR¹¹C(═O)X₁ₐX₂ₐC(═O)(CH₂)ₘO(CH₂)ₘC(═O)—; —**C(═O)O(CH₂)ₘNR¹¹C(═O)X₄C(═O)NR¹¹ (CH₂)ₘNR¹¹C(═O)(CH₂)ₘO(CH₂)ₘ—; —**C(═O)O(CH₂)ₘNR¹¹C(═O)X₅C(═O)(CH₂)ₘNR¹¹C(═O)(CH₂)ₘ—; —**C(═O)X₄C(═O)NR¹¹(CH₂)ₘNR¹¹C(═O)(CH₂)ₘO(CH₂)ₘ—; —**C(═O)(CH₂)ₘNR¹¹C(═O)X₁ₐX₂ₐC(═O)(CH₂)ₘ—; —**C(═O)O(CH₂)ₘX₆C(═O)X₁ₐX₂ₐC(═O)(CH₂)ₘ—; —**C(═O)(CH₂)ₘNR¹¹C(═O)((CH₂)ₘO)ₙ(CH₂)ₘ—**; —**C(═O)O(CH₂)ₘX₆C(═O)(CH₂)ₘ—; —**C(═O)O(CH₂)ₘX₆C(═O)(CH₂)ₘO(CH₂)ₘ—; —**C(═O)O(CH₂)ₘX₆C(═O)X₁ₐX₂ₐC(═O)(CH₂)ₘ—; —**C(═O)O(CH₂)ₘX₆C(═O)X₁ₐX₂ₐC(═O)(CH₂)ₘO(CH₂)ₘ—; —**C(═O)O(CH₂)ₘX₆C(═O)X₁ₐX₂ₐC(═O)(CH₂)ₘO(CH₂)ₘC(═O)—; —**C(═O)O(CH₂)ₘX₆C(═O)X₄C(═O)NR¹¹ (CH₂)ₘNR¹¹C(═O)(CH₂)ₘO(CH₂)ₘ—; —**C(═O)X₄C(═O)X₆ (CH₂)ₘNR¹¹C(═O)(CH₂)ₘO(CH₂)ₘ—; —**C(═O)(CH₂)ₘX₆C(═O)X₁ₐX₂ₐC(═O)(CH₂)ₘ—; —**C(═O)O((CH₂)ₘO)ₙ(CH₂)ₘNR¹¹C(═O)X₅C(═O)(CH₂)ₘ—; —**C(═O)O((CH₂)ₘO)ₙ(CH₂)ₘNR¹¹C(═O)X₅C(═O)(CH₂)ₘNR¹¹C(═O)(CH₂)ₘ—; —**C(═O)O((CH₂)ₘO)ₙ(CH₂)ₘNR¹¹C(═O)X₅C(═O)(CH₂)ₘX₃(CH₂)ₘ—; —**C(═O)O(CH₂)ₘO)ₙ(CH₂)ₘNR¹¹C(═O)X₅C(═O)(CH₂)ₘO)ₙ(CH₂)ₘ—; —**C(═O)O(CH₂)ₘO)ₙ(CH₂)ₘNR¹¹C(═O)X₅C(═O)(CH₂)ₘO)ₙ(CH₂)ₘNR¹¹C(═O)(CH₂)ₘ—; —**C(═O)O((CH₂)ₘO)ₙ(CH₂)ₘNR¹¹C(═O)X₅C(═O)(CH₂)ₘO)ₙ(CH₂)ₘNR¹¹C(═O)(CH₂)ₘX₃(CH₂)ₘ—; —**C(═O)O((CH₂)ₘO)ₙ(CH₂)ₘNR¹¹C(═O))X₅C(═O)((CH₂)ₘO)ₙ(CH₂)ₘX₃(CH₂)ₘ—; —**C(═O)O((CH₂)ₘO)ₙ(CH₂)ₘNR¹¹C(═O)X₅C(═O)(CH₂)ₘNR¹¹C(═O)((CH₂)ₘO)ₙ(CH₂)ₘ—; —**C(═O)O((CH₂)ₘO)ₙ(CH₂)ₘNR¹¹C(═O)X₅C(═O)(CH₂)ₘNR¹¹C(═O)((CH₂)ₘO)ₙ(CH₂)ₘX₃(CH₂)ₘ—; —**C(═O)O((CH₂)ₘO)ₙ(CH₂)ₘNR¹¹C(═O)X₅(CH₂)ₘX₃(CH₂)ₘ—; —**C(═O)O((CH₂)ₘO)ₙ(CH₂)ₘNR¹¹C(═O)X₅((CH₂)ₘO)ₙ(CH₂)ₘ—; —**C(═O)O((CH₂)ₘO)ₙ(CH₂)ₘNR¹¹C(═O)X₅((CH₂)ₘO)ₙ(CH₂)ₘNR¹¹C(═O)(CH₂)ₘ—; —**C(═O)O((CH₂)ₘO)ₙ(CH₂)ₘNR¹¹C(═O)X₅((CH₂)ₘO)ₙ(CH₂)ₘNR¹¹C(═O)(CH₂)ₘX₃(CH₂)ₘ—; —**C(═O)O((CH₂)ₘO)ₙ(CH₂)ₘNR¹¹C(═O)X₅((CH₂)ₘO)ₙ(CH₂)ₘX₃(CH₂)ₘ—; —**C(═O)O((CH₂)ₘO)ₙ(CH₂)ₘNR¹¹C(═O)X₅(CH₂)ₘNR¹¹ ((CH₂)ₘO)ₙ(CH₂)ₘ—; —**C(═O)O((CH₂)ₘO)ₙ(CH₂)ₘNR¹¹C(═O)X₅C(═O)(CH₂)ₘNR¹¹ (CH₂)ₘO)ₙ(CH₂)ₘX₃(CH₂)ₘ—; —**C(═O)O((CH₂)ₘO)ₙ(CH₂)ₘNR¹¹C(═O)X₅(CH₂)ₘ—; —**C(═O)O((CH₂)ₘO)ₙ(CH₂)ₘNR¹¹C(═O)X₅C(═O)((CH₂)ₘO)ₙ(CH₂)ₘ—; —**C(═O)O((CH₂)ₘO)ₙ(CH₂)ₘNR¹¹C(═O)X₅(CH₂)ₘX₃(CH₂)ₘ—; —**C(═O)O(CH₂)ₘ—; —**C(═O)O((CH₂)ₘO)ₙ(CH₂)ₘ—; —**C(═O)O(CH₂)ₘNR¹¹(CH₂)ₘ—; —**C(═O)O(CH₂)ₘNR¹¹(CH₂)ₘC(═O)X₂ₐX₁ₐC(═O)—; —**C(═O)O(CH₂)ₘX₃(CH₂)ₘ—; —**C(═O)O((CH₂)ₘO)ₙ(CH₂)ₘX₃(CH₂)ₘ—; —**C(═O)O((CH₂)ₘO)ₙ(CH₂)ₘNR¹¹C(═O)(CH₂)ₘ—; —**C(═O)O(CH₂)ₘNR¹¹C(═O(CH₂)ₘX₃(CH₂)ₘ—; —**C(═O)O((CH₂)ₘO)—(CH₂)ₘNR¹¹C(═O)(CH₂)ₘX₃(CH₂)ₘ—; —**C(═O)O(CH₂)ₘO)ₙX₃(CH₂)ₘ—; —**C(═O)O(CH₂)ₘO)—(CH₂)ₘX₃(CH₂)ₘ—; —**C(═O)O((CH₂)ₘO)—(CH₂)ₘC(═O)NR¹¹(CH₂)ₘ—; —**C(═O)O(CH₂)ₘC(R¹²)₂—; —**C(═O)OCH₂)ₘC(R¹²)₂SS(CH₂)ₘNR¹¹C(═O)(CH₂)ₘ—, and
—**C(═O)O(CH₂)ₘC(═O)NR¹¹(CH₂)ₘ—, where: ** indicates point of attachment to the drug moiety (D) and the other end can be connected to R¹⁰⁰, i.e., the coupling group as described herein;
wherein:
X₁ₐ is

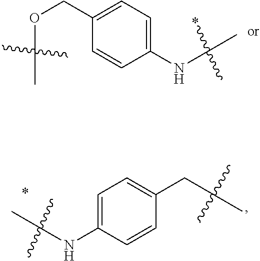

where the * indicates the point of attachment to X₂ₐ;
X₂ₐ is selected from

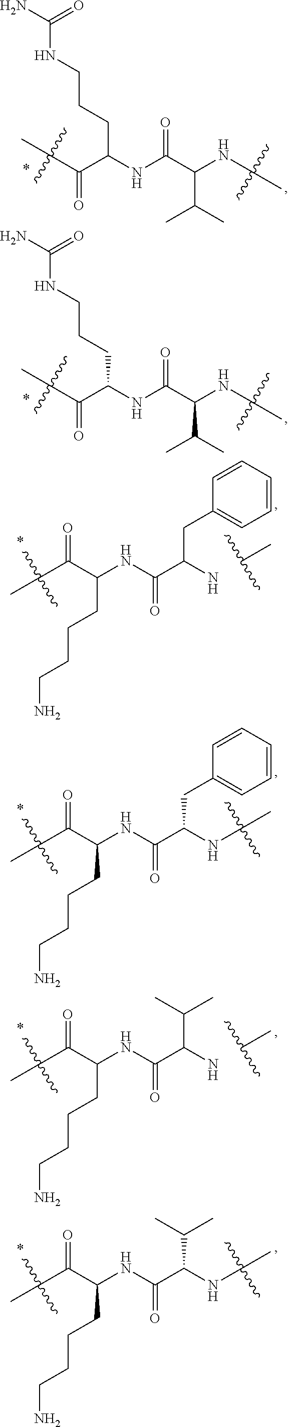

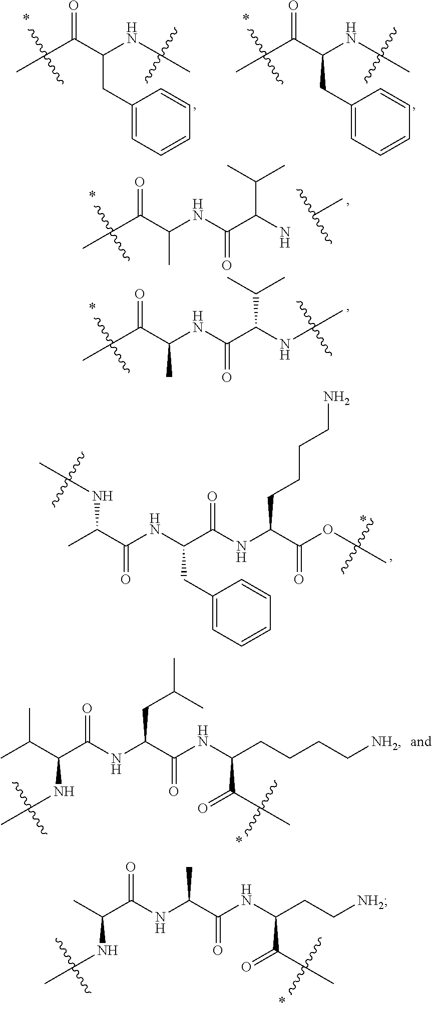

where the * indicates the point of attachment to X₁ₐ;
X₃ is

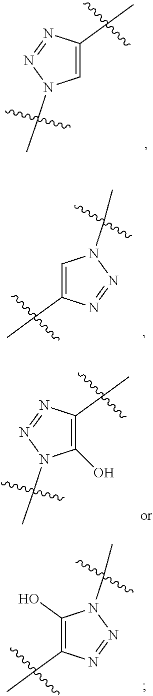 [SMILES: *c1cn(*)nn1 ; *c1cn(*)nn1 ; *c1nnn(*)c1O ; *c1nnn(*)c1O]

X₄ is —O(CH₂)ₙSSC(R¹²)₂(CH₂)ₙ— or —(CH₂)ₙC(R¹²)₂SS(CH₂)ₙO—;
X₅ is

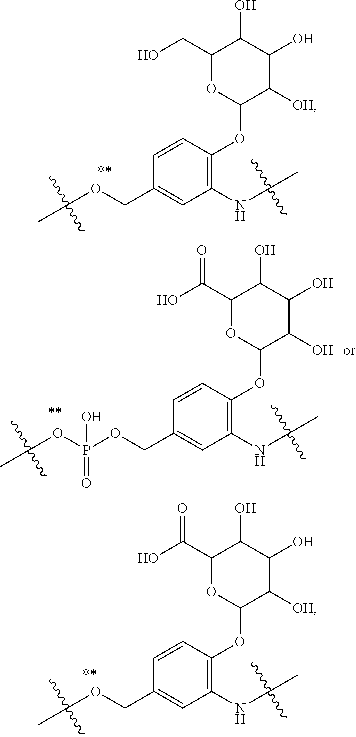 [SMILES: *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the ** indicates orientation toward the Drug moiety;
X₆ is

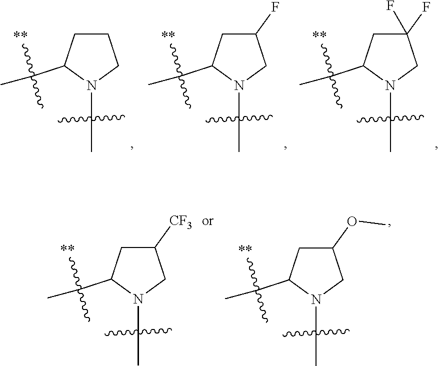

where the ** indicates orientation toward the Drug moiety;
each R¹¹ is independently selected from H and C₁-C₆alkyl;
each R¹² is independently selected from H and C₁-C₆alkyl;
each m is independently selected from 1, 2, 3, 4, 5, 6, 7, 8, 9 and 10, and
each n is independently selected from 1, 2, 3, 4, 5, 6, 7, 8, 9, 10, 11, 12, 13, 14, 15, 16,17 and 18.

### Methods of Conjugation

The present invention provides various methods of conjugating Linker-Drug groups of the invention to antibodies or antibody fragments to produce Antibody Drug Conjugates which comprise a linker having one or more hydrophilic moieties.

A general reaction scheme for the formation of Antibody Drug Conjugates of Formula (E′) is shown in Scheme 2 below:

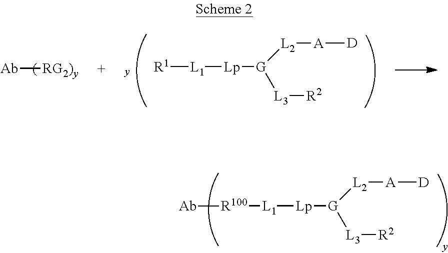

where: RG₂ is a reactive group which reacts with a compatible R¹ group to form a corresponding R¹⁰⁰ group (such groups are illustrated in Table 3 and Table 4). D, R¹, L₁, Lp, L₂, L₃, R², A, G, Ab, y and R¹⁰⁰ are as defined herein.

Scheme 3 further illustrates this general approach for the formation of Antibody Drug Conjugates of Formula (E′), wherein the antibody comprises reactive groups (RG₂) which react with an R¹ group (as defined herein) to covalently attach the Linker-Drug group to the antibody via an R¹⁰⁰ group (as defined herein). For illustrative purposes only Scheme 3 shows the antibody having four RG₂ groups.

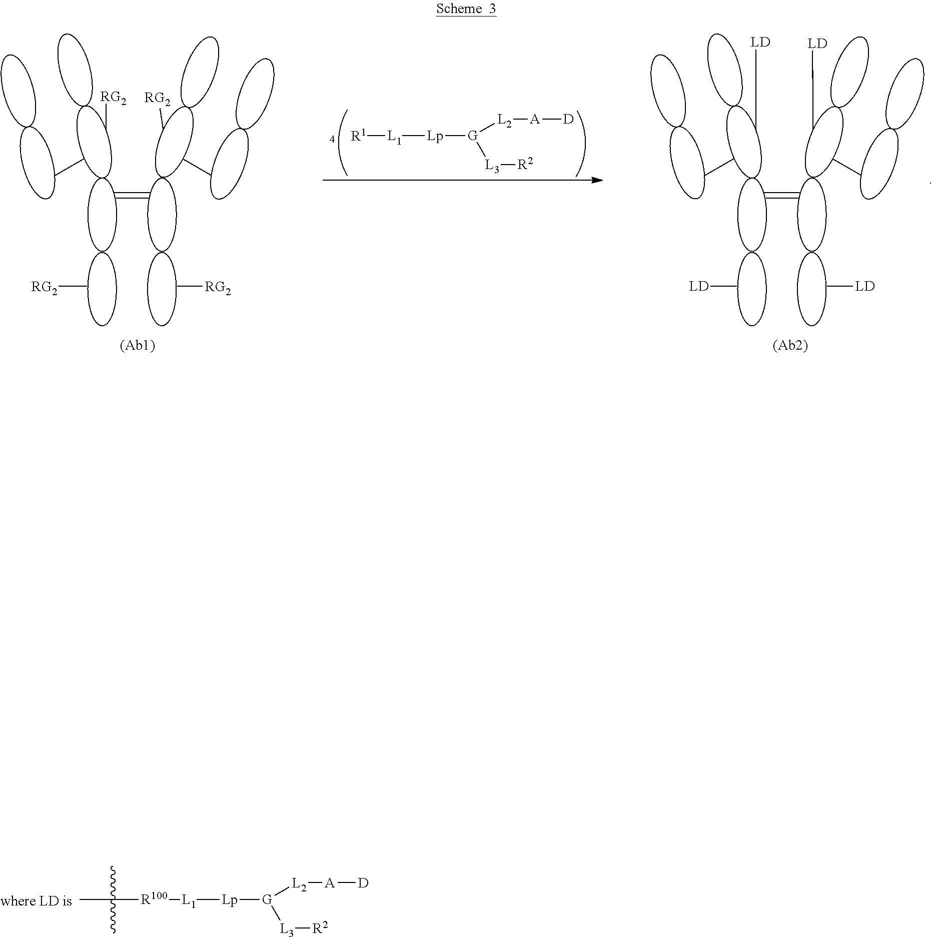

In one aspect, Linker-Drug groups are conjugated to antibodies via modified cysteine residues in the antibodies (see for example WO2014/124316). Scheme 4 illustrates this approach for the formation of Antibody Drug Conjugates of Formula (E′) wherein a free thiol group generated from the engineered cysteine residues in the antibody react with an R¹ group (where R¹ is a maleimide) to covalently attach the Linker-Drug group to the antibody via an R¹⁰⁰ group (where R¹⁰⁰ is a succinimide ring). For illustrative purposes only Scheme 4 shows the antibody having four free thiol groups.

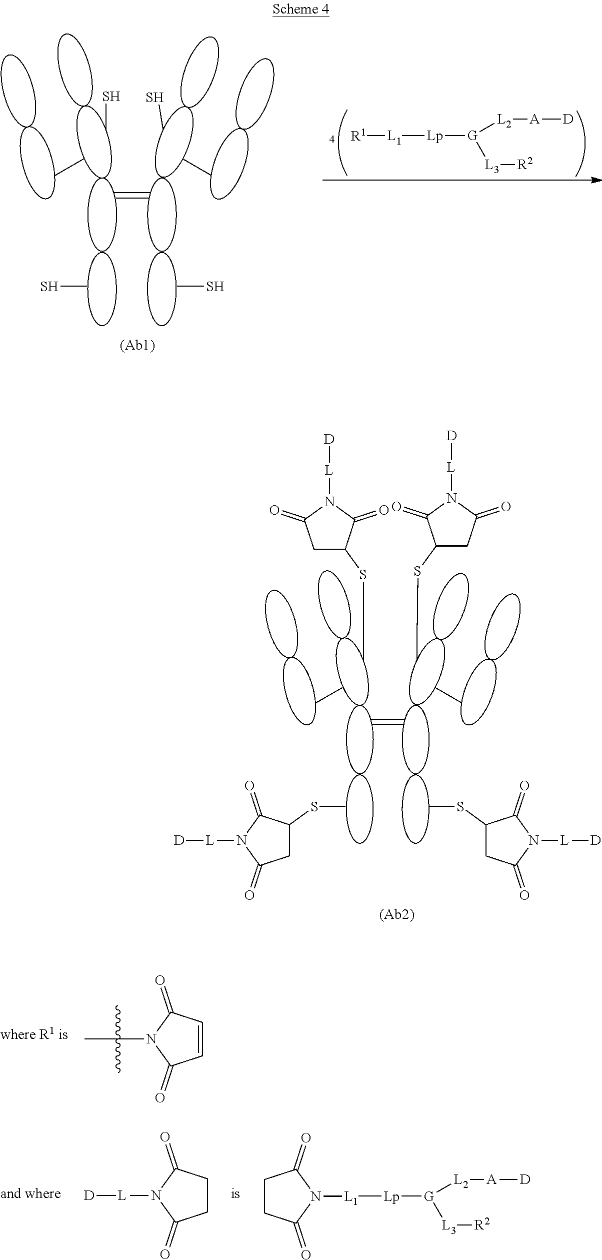

In another aspect, Linker-Drug groups are conjugated to antibodies via lysine residues in the antibodies. Scheme 5 illustrates this approach for the formation of Antibody Drug Conjugates of Formula (E′) wherein a free amine group from the lysine residues in the antibody react with an R¹ group (where R¹ is an NHS ester, a pentafluorophenyl or a tetrafluorophenyl) to covalently attach the Linker-Drug group to the antibody via an R¹⁰⁰ group (where R¹⁰⁰ is an amide). For illustrative purposes only Scheme 5 shows the antibody having four amine groups.

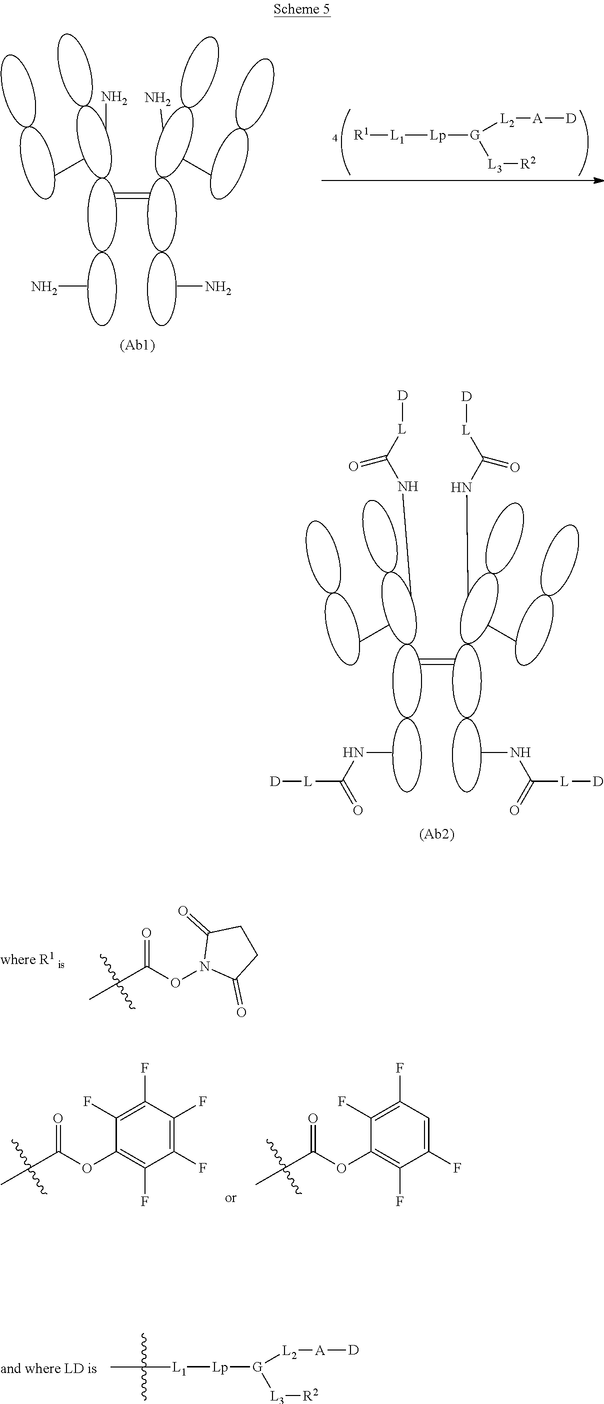

In another aspect, Linker-Drug groups are conjugated to antibodies via formation of an oxime bridge at the naturally occurring disulfide bridges of an antibody. The oxime bridge is formed by initially creating a ketone bridge by reduction of an interchain disulfide bridge of the antibody and re-bridging using a 1,3-dihaloacetone (e.g. 1,3-dichloroacetone). Subsequent reaction with a Linker-Drug group comprising a hydroxyl amine thereby form an oxime linkage (oxime bridge) which attaches the Linker-Drug group to the antibody (see for example WO2014/083505). Scheme 6 illustrates this approach for the formation of Antibody Drug Conjugates of Formula (E′).

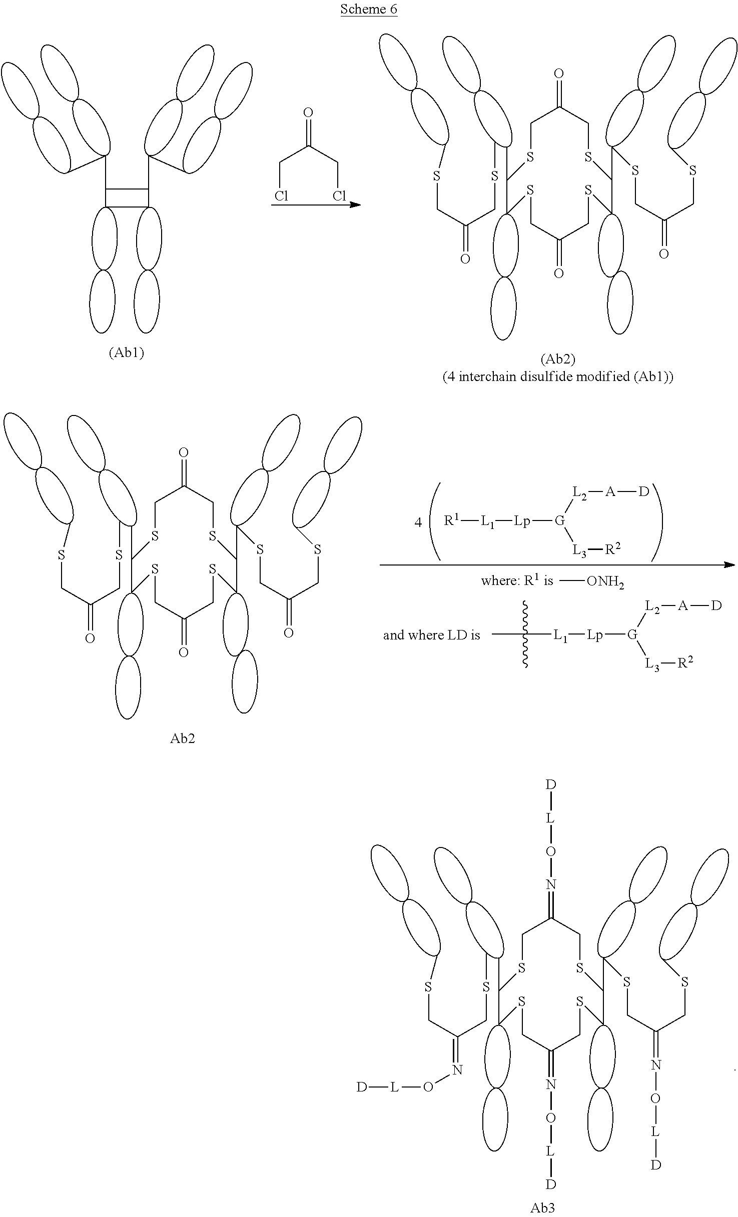

A general reaction scheme for the formation of Antibody Drug Conjugates of Formula (F′) is shown in Scheme 7 below:

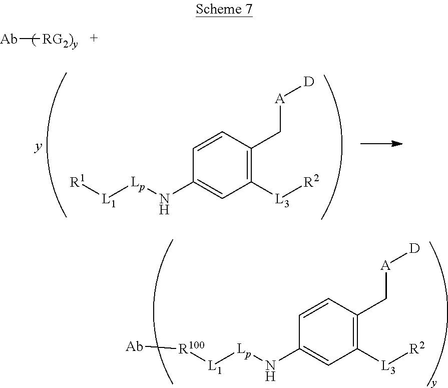

where: RG₂ is a reactive group which reacts with a compatible R¹ group to form a corresponding R¹⁰⁰ group (such groups are illustrated in Table 3 and Table 4). D, R¹, L₁, Lp, Ab, y and R¹⁰⁰ are as defined herein.

Scheme 8 further illustrates this general approach for the formation of Antibody Drug Conjugates of Formula (F′), wherein the antibody comprises reactive groups (RG₂) which react with an R¹ group (as defined herein) to covalently attach the Linker-Drug group to the antibody via an R¹⁰⁰ group (as defined herein). For illustrative purposes only Scheme 8 shows the antibody having four RG₂ groups.

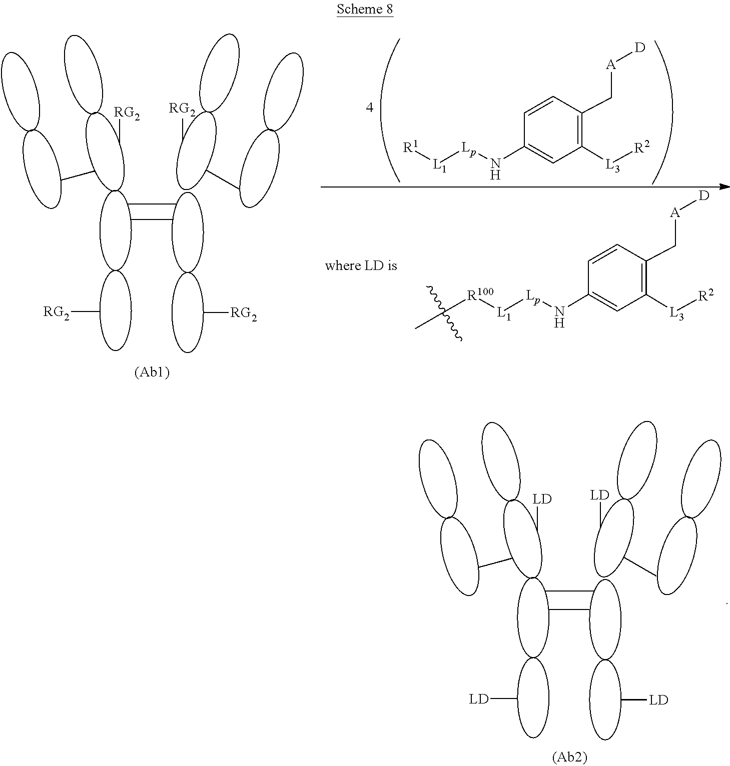

In one aspect, Linker-Drug groups are conjugated to antibodies via modified cysteine residues in the antibodies (see for example WO2014/124316). Scheme 9 illustrates this approach for the formation of Antibody Drug Conjugates of Formula (F′) wherein a free thiol group generated from the engineered cysteine residues in the antibody react with an R¹ group (where R¹ is a maleimide) to covalently attach the Linker-Drug group to the antibody via an R¹⁰⁰ group (where R¹⁰⁰ is a succinimide ring). For illustrative purposes only Scheme 9 shows the antibody having four free thiol groups.

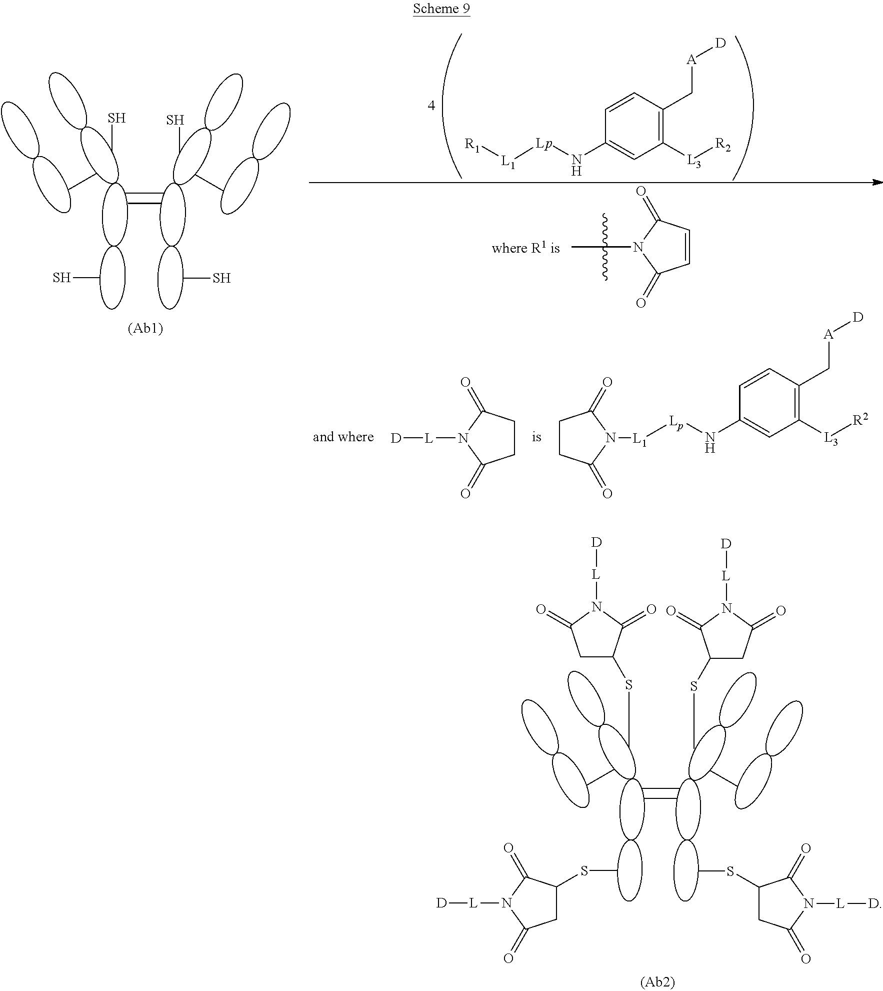

In another aspect, Linker-Drug groups are conjugated to antibodies via lysine residues in the antibodies. Scheme 10 illustrates this approach for the formation of Antibody Drug Conjugates of Formula (F′) wherein a free amine group from the lysine residues in the antibody react with an R¹ group (where R¹ is an NHS ester, a pentafluorophenyl or a tetrafluorophenyl) to covalently attach the Linker-Drug group to the antibody via an R¹⁰⁰ group (where R¹⁰⁰ is an amide). For illustrative purposes only Scheme 10 shows the antibody having four amine groups.

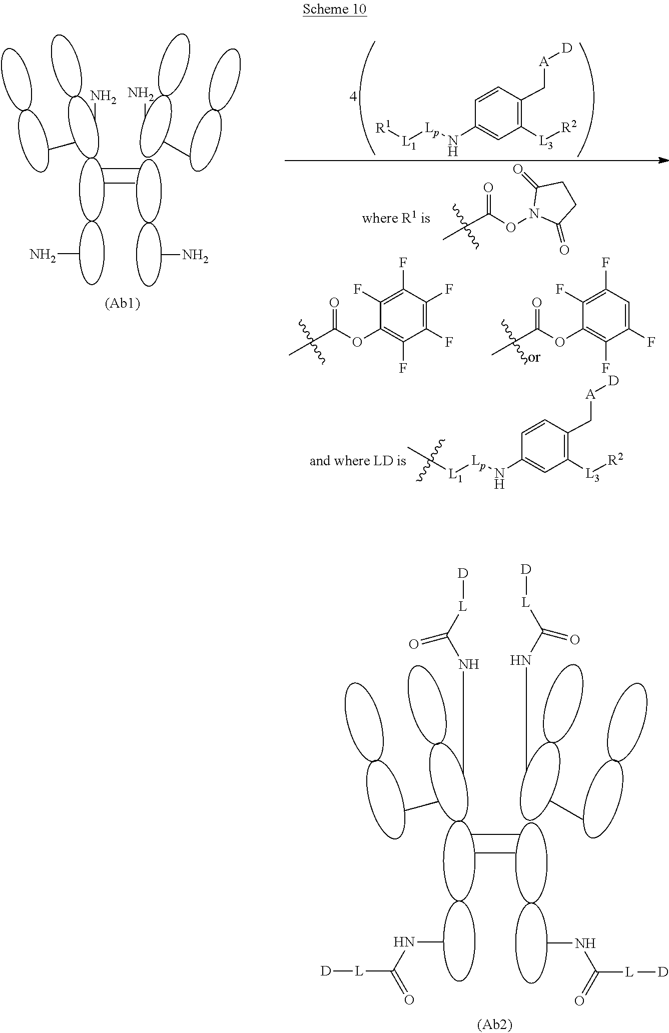

In another aspect, Linker-Drug groups are conjugated to antibodies via formation of an oxime bridge at the naturally occurring disulfide bridges of an antibody. The oxime bridge is formed by initially creating a ketone bridge by reduction of an interchain disulfide bridge of the antibody and re-bridging using a 1,3-dihaloacetone (e.g. 1,3-dichloroacetone). Subsequent reaction with a Linker-Drug group comprising a hydroxyl amine thereby form an oxime linkage (oxime bridge) which attaches the Linker-Drug group to the antibody (see for example WO2014/083505). Scheme 11 illustrates this approach for the formation of Antibody Drug Conjugates of Formula (F′).

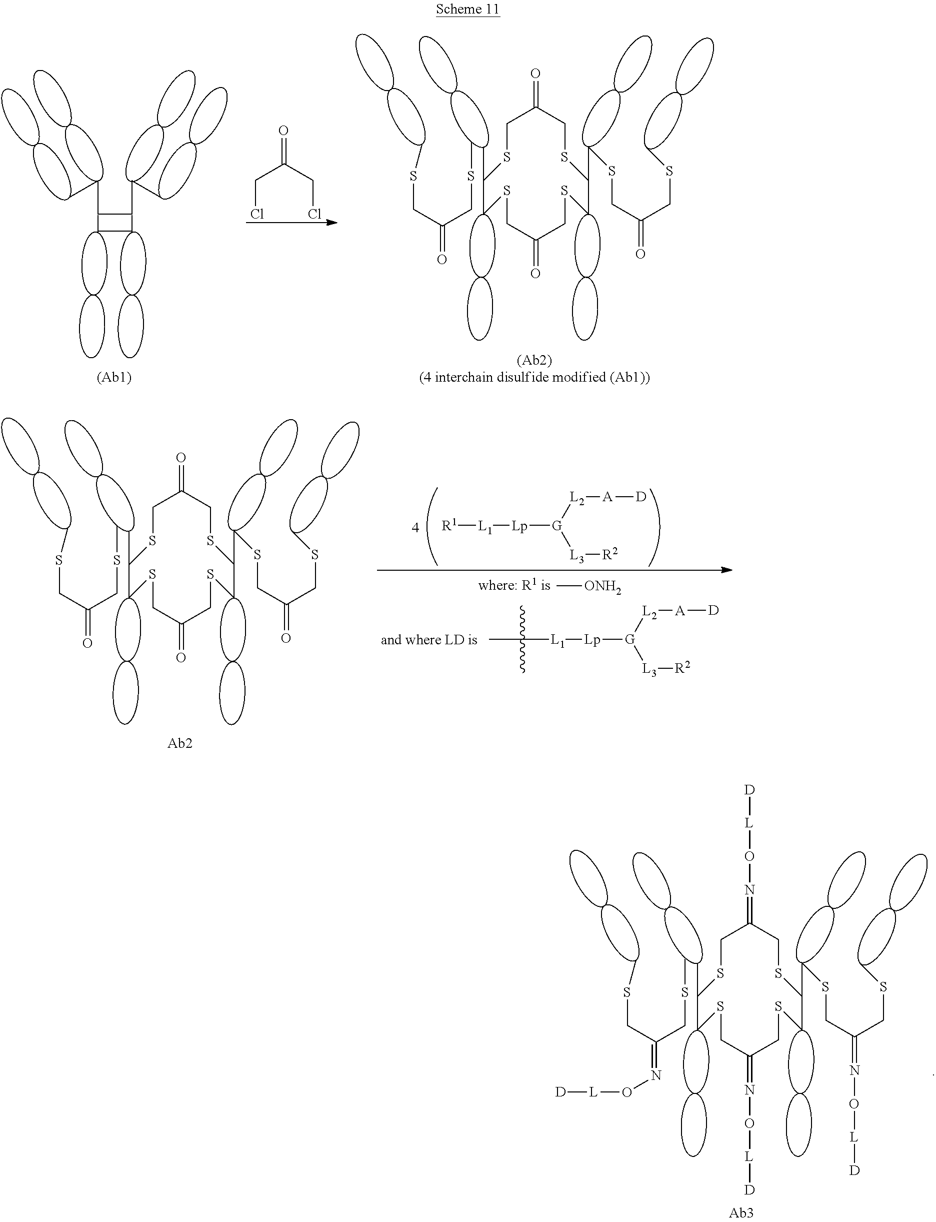

Provided are also protocols for some aspects of analytical methodology for evaluating antibody conjugates of the invention. Such analytical methodology and results can demonstrate that the conjugates have favorable properties, for example properties that would make them easier to manufacture, easier to administer to patients, more efficacious, and/or potentially safer for patients. One example is the determination of molecular size by size exclusion chromatography (SEC) wherein the amount of desired antibody species in a sample is determined relative to the amount of high molecular weight contaminants (e.g., dimer, multimer, or aggregated antibody) or low molecular weight contaminants (e.g., antibody fragments, degradation products, or individual antibody chains) present in the sample. In general, it is desirable to have higher amounts of monomer and lower amounts of, for example, aggregated antibody due to the impact of, for example, aggregates on other properties of the antibody sample such as but not limited to clearance rate, immunogenicity, and toxicity. A further example is the determination of the hydrophobicity by hydrophobic interaction chromatography (HIC) wherein the hydrophobicity of a sample is assessed relative to a set of standard antibodies of known properties. In general, it is desirable to have low hydrophobicity due to the impact of hydrophobicity on other properties of the antibody sample such as but not limited to aggregation, aggregation over time, adherence to surfaces, hepatotoxicity, clearance rates, and pharmacokinetic exposure. See Damle, N. K., Nat Biotechnol. 2008; 26 (8):884-885; Singh, S. K., Pharm Res. 2015; 32 (11):3541-71. When measured by hydrophobic interaction chromatography, higher hydrophobicity index scores (i.e. elution from HIC column faster) reflect lower hydrophobicity of the conjugates. As shown in Examples below, a majority of the tested antibody conjugates showed a hydrophobicity index of greater than 0.8. In some embodiments, provided are antibody conjugates having a hydrophobicity index of 0.8 or greater, as determined by hydrophobic interaction chromatography.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example 1. Synthesis and Characterization of Bcl-xL Payloads

Exemplary payloads were synthesized using exemplary methods described in this example. All reagents obtained from commercial sources were used without further purification. Anhydrous solvents were obtained from commercial sources and used without further drying.

Column Chromatography: Automated flash column chromatography was performed on ISCO CombiFlash® Rf 200 or CombiFlash® Rf+ Lumen™ using RediSep® Rf Normal-phase Silica Flash Columns (35-70 μm, 60 Å), RediSep Rf Gold® Normal-phase Silica High Performance Columns (20-40 μm, 60 Å), RediSep® Rf Reversed-phase C18 Columns (40-63 μm, 60 Å), or RediSep Rf Gold® Reversed-phase C18 High Performance Columns (20-40 μm, 100 Å).

TLC: Thin layer chromatography was conducted with 5×10 cm plates coated with Merck Type 60 F₂₅₄ silica-gel.

Microwave Reactions: Microwave heating was performed with a CEM Discover® SP, or with an Anton Paar Monowave Microwave Reactor.

NMR: ¹H-NMR measurements were performed on a Bruker Avance III 500 MHz spectrometer, a Bruker Avance III 400 MHz spectrometer, or a Bruker DPX-400 spectrometer using DMSO-d₆ or CDCl₃ as solvent. ¹H NMR data is in the form of delta values, given in part per million (ppm), using the residual peak of the solvent (2.50 ppm for DMSO-de and 7.26 ppm for CDCl₃) as internal standard. Splitting patterns are designated as: s (singlet), d (doublet), t (triplet), q (quartet), quint (quintet), sept (septet), m (multiplet), br s (broad singlet), dd (doublet of doublets), td (triplet of doublets), dt (doublet of triplets), ddd (doublet of doublet of doublets).

Analytical LC-MS: Certain compounds of the present invention were characterized by high performance liquid chromatography-mass spectroscopy (HPLC-MS) on Agilent HP1200 with Agilent 6140 quadrupole LC/MS, operating in positive or negative ion electrospray ionisation mode. Molecular weight scan range is 100 to 1350. Parallel UV detection was done at 210 nm and 254 nm. Samples were supplied as a 1 mM solution in ACN, or in THF/H₂O (1:1) with 5 μL loop injection. LCMS analyses were performed on two instruments, one of which was operated with basic, and the other with acidic eluents.

Basic LCMS: Gemini-NX, 3 μm, C18, 50 mm×3.00 mm i.d. column at 23° C., at a flow rate of 1 mL min-1 using 5 mM ammonium bicarbonate (Solvent A) and acetonitrile (Solvent B) with a gradient starting from 100% Solvent A and finishing at 100% Solvent B over various/certain duration of time.

Acidic LCMS: KINATEX XB-C18-100A, 2.6 μm, 50 mm*2.1 mm column at 40° C., at a flow rate of 1 mL min−1 using 0.02% v/v aqueous formic acid (Solvent A) and 0.02% v/v formic acid in acetonitrile (Solvent B) with a gradient starting from 100% Solvent A and finishing at 100% Solvent B over various/certain duration of time.

Certain other compounds of the present invention were characterized HPLC-MS under specific named methods as follows. For all of these methods UV detection was by diode array detector at 230, 254, and 270 nm. Sample injection volume was 1 μL. Gradient elutions were run by defining flow rates and percentage mixtures of the following mobile phases, using HPLC-grade solvents:
Solvent A: 10 mM aqueous ammonium formate+0.04% (v/v) formic acid
Solvent B: Acetonitrile+5.3% (v/v) Solvent A+0.04% (v/v) formic acid.

Retention times (RT) for these named methods are reported in minutes. Ionisation is recorded in positive mode, negative mode, or positive-negative switching mode. Specific details for individual methods follow.

LCMS-V-B methods: Using an Agilent 1200 SL series instrument linked to an Agilent MSD 6140 single quadrupole with an ESI-APCI multimode source (Methods LCMS-V-B1 and LCMS-V-B2) or using an Agilent 1290 Infinity II series instrument connected to an Agilent TOF 6230 with an ESI-jet stream source (Method LCMS-V-B1); column: Thermo Accucore 2.6 μm, C18, 50 mm×2.1 mm at 55° C. Gradient details for methods LCMS-V-B1 and LCMS-V-B2 are shown in the Table 5 below:

TABLE 5
Gradient Details for Methods LCMS-V-B1 and LCMS-V-B2

| | |
|---|---|
| LCMS-V-B1 | LCMS-V-B2 |

| Time | Solvent | Solvent | Solvent | Solvent | Flow |
|---|---|---|---|---|---|
| (min) | A (%) | B (%) | A (%) | B (%) | (mL/min) |
| 0 | 95 | 5 | 60 | 40 | 1.1 |
| 0.12 | 95 | 5 | 60 | 40 | 1.3 |
| 1.30 | 5 | 95 | 2 | 98 | 1.3 |
| 1.35 | 5 | 95 | 2 | 98 | 1.6 |
| 1.85 | 5 | 95 | 2 | 98 | 1.6 |
| 1.90 | 5 | 95 | 2 | 98 | 1.3 |
| 1.95 | 95 | 5 | 95 | 5 | 1.3 |

LCMS-V-C method: Using an Agilent 1200 SL series instrument linked to an Agilent MSD 6140 single quadrupole with an ESI-APCI multimode source; column: Agilent Zorbax Eclipse plus 3.5 μm, C18(2), 30 mm×2.1 mm at 35° C. Gradient details for method LCMS-V-C are shown in Table 6 below:

TABLE 6
Gradient Details for Method LCMS-V-C

| Time | Solvent | Solvent | Flow |
|---|---|---|---|
| (min) | A (%) | B (%) | (mL/min) |
| 0 | 95 | 5 | 1 |
| 0.25 | 95 | 5 | 1 |
| 2.50 | 95 | 5 | 1 |
| 2.55 | 5 | 95 | 1.7 |
| 3.60 | 5 | 95 | 1.7 |
| 3.65 | 5 | 95 | 1 |
| 3.70 | 95 | 5 | 1 |
| 3.75 | 95 | 5 | 1 |

Preparative HPLC: Certain compounds of the present invention were purified by high performance liquid chromatography (HPLC) on an Armen Spot Liquid Chromatography or Teledyne EZ system with a Gemini-NX® 10 μM C18, 250 mm×50 mm i.d. column running at a flow rate of 118 mL min-1 with UV diode array detection (210-400 nm) using 25 mM aqueous NH₄HCO₃ solution and MeCN or 0.1% TFA in water and MeCN as eluents.

Certain other compounds of the present invention were purified by HPLC under specific named methods as follows:

HPLC-V-A methods: These were performed on a Waters FractionLynx MS autopurification system, with a Gemini® 5 μm C18(2), 100 mm×20 mm i.d. column from Phenomenex, running at a flow rate of 20 cm³ min⁻¹ with UV diode array detection (210-400 nm) and mass-directed collection. The mass spectrometer was a Waters Micromass ZQ2000 spectrometer, operating in positive or negative ion electrospray ionisation modes, with a molecular weight scan range of 150 to 1000.

Method HPLC-V-A1 (pH 4): Solvent A: 10 mM aqueous ammonium acetate+0.08% (v/v) formic acid; Solvent B: acetonitrile+5% (v/v) Solvent A+0.08% (v/v) formic acid

Method HPLC-V-A2 (pH 9): Solvent A: 10 mM aqueous ammonium acetate+0.08% (v/v) conc. ammonia; Solvent B: acetonitrile+5% (v/v) Solvent A+0.08% (v/v) conc. ammonia

HPLC-V-B methods: Performed on an AccQPrep HP125 (Teledyne ISCO) system, with a Gemini® NX 5 μm C18(2), 150 mm×21.2 mm i.d. column from Phenomenex, running at a flow rate of 20 cm³ min⁻¹ with UV (214 and 254 nm) and ELS detection.

Method HPLC-V-B1 (pH 4): Solvent A: water+0.08% (v/v) formic acid; solvent B: acetonitrile+0.08% (v/v) formic acid.

Method HPLC-V-B2 (pH 9): Solvent A: water+0.08% (v/v) conc. ammonia; solvent B: acetonitrile+0.08% (v/v) conc. ammonia.

Method HPLC-V-B3 (neutral): Solvent A: water; Solvent B: acetonitrile.

Analytical GC-MS: Combination gas chromatography and low resolution mass spectrometry (GC-MS) was performed on Agilent 6850 gas chromatograph and Agilent 5975C mass spectrometer using 15 m×0.25 mm column with 0.25 μm HP-5 MS coating and helium as carrier gas. Ion source: El+, 70 eV, 230° C., quadrupole: 150° C., interface: 300° C.

High-resolution MS: High-resolution mass spectra were acquired on an Agilent 6230 time-of-flight mass spectrometer equipped with a Jet Stream electrospray ion source in positive ion mode. Injections of 0.5 μl were directed to the mass spectrometer at a flow rate 1.5 ml/min (5 mM ammonium-formate in water and acetonitrile gradient program), using an Agilent 1290 Infinity HPLC system. Jet Stream parameters: drying gas (N2) flow and temperature: 8.0 l/min and 325° C., respectively; nebulizer gas (N2) pressure: 30 psi; capillary voltage: 3000 V; sheath gas flow and temperature: 325° C. and 10.0 l/min; TOFMS parameters: fragmentor voltage: 100 V; skimmer potential: 60 V; OCT 1 RF Vpp:750 V. Full-scan mass spectra were acquired over the m/z range 105-1700 at an acquisition rate of 995.6 ms/spectrum and processed by Agilent MassHunter B.04.00 software.

Chemical naming: IUPAC-preferred names were generated using ChemAxon's ‘Structure to Name’ (s2n) functionality within MarvinSketch or JChem for Excel (JChem versions 16.6.13-18.22.3), or with the chemical naming functionality provided by Biovia® Draw 4.2.

Abbreviations

| | |
|---|---|
| Ahx | 6-hexanoic acid monomer |
| Boc | tert-butyloxycarbonyl |
| Boc₂O | di-tert-butyl dicarbonate |
| AgOTf | silver trifluoromethanesulfonate |
| ^{t}BuOH | tert-butanol |
| cc. or conc. | concentrated |
| CyOH | cyclohexanol |
| dba | (1E,4E)-1,5-diphenylpenta-1,4-dien-3-one,dibenzylideneacetone |
| DCM | dichloromethane |
| DEA | diethanolamine |
| DIAD | diisopropylazodicarboxylate |
| DIBAL-H | diisobutylaluminium hydride |
| DIPA | N-isopropylpropan-2-amine,diisopropylamine |
| DIPEA | N-ethyl-N-isopropyl-propan-2-amine,diisopropylethylamine |
| DMAP | 4-dimethylaminopyridine |
| ee. | enatiomeric excess |
| eq. | equivalent |
| EtO₂ | diethyl ether |
| EtOAc | ethyl acetate |
| HF × Pyr | Hydrogen fluoride pyridine |
| hs | homo sapiens |
| LDA | lithium diisopropylamide |
| MeCN | acetonitrile |
| MeOH | methanol |
| MTBE | methyl tert-butyl ether |
| NMP | N-methyl-2-pyrrolidone |
| Pd(AtaPhos)₂Cl₂ | bis(di-tert-butyl(4-dimethylaminophenyl)phos- |
| | phine)dichloropalladium(II) |
| PPh₃ | triphenylphosphine |
| rt | room temperature |
| RT | retention time (in minutes) |
| on | overnight |
| Pd\C | palladium on carbon |
| TBAF | tetrabutylammonium fluoride |
| TBAOH | tetrabutylammonium hydroxide |
| TBDPS-Cl | tert-butyl-chloro-diphenyl-silane |
| TBSCl | tert-butyl-chloro-dimethyl-silane |
| TEA | N,N-diethylethanamine |
| TFA | 2,2,2-trifluoroacetic acid |
| pTSA | 4-methylbenzenesulfonic acid |
| THF | tetrahydrofuran |
| TIPSCl | chloro(triisopropyl)silane |
| TMP-MgCl | 2,2,6,6-tetramethylpiperidinylmagnesium |
| | chloride lithium chloride complex solution |
| DIAD | diisopropylazodicarboxylate |
| Xantphos | 4,5-Bis(diphenylphosphino)-9,9-dimethylxanthene |
| BrettPhos | 2-(Dicyclohexylphosphino)-3,6-dimethoxy-2′,4′,6′- |
| | triisopropyl-1,1′-biphenyl |
| JosiPhos | (2R)-1-[(1 R)-1-(Dicyclohexylphosphino)ethyl]-2- |
| | (diphenylphosphino)ferrocene |
| JosiPhos Pd G₃ | {(R)-1-[(Sp)-2-(Dicyclohexylphosphino)ferrocenyl]ethyldi- |
| | tert-butylphosphine}[2-(2′-amino-1,1′- |
| | biphenyl)]palladium(II) methanesulfonate |
| Xantphos Pd G₃ | [(4,5-Bis(diphenylphosphino)-9,9-dimethylxanthene)-2-(2′- |
| | amino-1,1′-biphenyl)]palladium(II) methanesulfonate |
| BINAP | 2,2′-Bis(diphenylphosphino)-1,1′-binaphthyl |
| rac-BINAP Pd G₃ | [(2,2′-Bis(diphenylphosphino)-1,1′-binaphthyl)-2-(2′-amino-1,1′- |
| | biphenyl)]palladium(II) methanesulfonate |
| Pd(dppf)Cl₂•CH₂Cl₂ | [1,1′-Bis(diphenylphosphino)ferrocene]dichloropalladium(II) |
| Pd₂(dba)₃ | Tris(dibenzylideneacetone)dipalladium(0) |
| Pd(PPh₃)₂Cl₂ | Bis(triphenylphosphine)palladium chloride |
| Pd(AtaPhos)₂Cl₂ | bis(di-tert-butyl(4-dimethylaminophenyl)phosphine)dichloropalladium(II) |

### Named General Procedures

The following are representative experimental procedures that are referred to by name in subsequent Preparations.

### Sonogashira General Procedure

The mixture of 1 eq. of aryl halogenide, 2 eq. of acetylene, 0.05 eq. of Pd(PPh₃)₂Cl₂, 0.05 eq. of CuI, and DIPA (1 mL/mmol) in THF (5 mL/mmol) was kept at 60° C. After reaching an appropriate conversion the volatiles were removed under reduced pressure, the crude intermediate was purified via flash chromatography using heptane/EtOAc as eluents.

Deprotection with HFIP General Procedure

Substrate in HFIP (10 mL/mmol) was kept at 100-120° C. in a pressure bottle. After reaching an appropriate conversion the volatiles were removed under reduced pressure, the crude intermediate was purified via flash chromatography using heptane/EtOAc as eluents.

### Deprotection and Hydrolysis General Procedure

The mixture of 1 eq. of substrate and 100 eq. of HF×Pyr in MeCN (15 mL/mmol) was stirred at 60° C. After reaching an appropriate conversion, the volatiles were removed under reduced pressure, the residue was suspended in a 1:1 mixture of THF—water (30 mL/mmol), 150 eq. of LiOH×H₂O was added, and the mixture was stirred at rt. After reaching an appropriate conversion, the volatiles were removed under reduced pressure; the crude product was purified via flash chromatography using DCM and MeOH (containing 1.2% NH₃) as eluents. In some alternative procedures, the 1:1 mixture of THF—water was replaced with a 1:1 mixture of 1,4-dioxane-water.

### Alkylation General Procedure

The mixture of 1 eq. of phenol/carbamate, 1-2 eq. of alkyl iodide/bromide, and 2-3 eq. of Cs₂CO₃ in acetone (5 mL/mmol) was stirred at rt for phenols and at 55° C. for carbamates. After reaching an appropriate conversion the volatiles were removed under reduced pressure, the crude intermediate was purified via flash chromatography (using heptane/EtOAc as eluents for instance) or reverse phase flash column chromatography.

Alkylation with Tosylate General Procedure

An oven-dried vial was equipped with a PTFE-coated magnetic stirring bar, and was charged with 1 eq. tosylate and 5 eq. of the appropriate amine suspended in MeCN (5 mL/mmol). The reaction mixture was then warmed up to 50° C. and stirred at that temperature until no further conversion was observed. The reaction mixture was diluted with DCM then it was injected onto a DCM preconditioned silica gel column. Then it was purified via flash chromatography using DCM and MeOH (1.2% NH₃) as eluents.

### Buchwald General Procedure I

The mixture of 1 eq. of chloro-substrate, 2 eq. of 1,3-benzothiazol-2-amine, 0.1 eq. of Pd₂(dba)₃, 0.2 eq. of XantPhos, and 3 eq. of DIPEA in CyOH (5 mL/mmol) was kept at 140° C. After reaching an appropriate conversion, the reaction mixture was diluted with DCM (10 mL/mmol), injected onto a preconditioned silica gel column and was purified via flash chromatography (using heptane/EtOAc as eluents for instance).

### Buchwald General Procedure II

The mixture of chloro compound, 2 eq. of 1,3-benzothiazol-2-amine, 10 mol % of JosiPhos Pd(G3) and 3 eq. of DIPEA suspended in 1,4-dioxane (5 mL/mmol) were stirred at reflux until no further conversion was observed. Celite was added to the reaction mixture and the volatiles were removed under reduced pressure. Then it was purified via flash chromatography on 120 g silica gel column using heptane-EtOAc or DCM-MeOH (1.2% NH₃) as eluents.

### Buchwald General Procedure III

The mixture of 1 eq. of thiazol amine, 1.2-1.5 eq. of (Z)—N-(6-chloro-4-methyl-pyridazin-3-yl)-3-(2-trimethylsilylethoxymethyl)-1,3-benzothiazol-2-imine, 3 eq. of Cs₂CO₃, 0.1 eq. of Pd₂(dba)₃, 0.2 eq. of XantPhos and 3 eq. of DIPEA in 1,4-dioxane (5 mL/mmol) was kept at reflux. After reaching an appropriate conversion the volatiles were removed under reduced pressure, the crude intermediate was purified via flash column chromatography.

### Mitsunobu General Procedure I

To the mixture of 1 eq. of aliphatic alcohol, 1 eq. of carbamate/phenol, and 1 eq. triphenylphosphine in toluene (5 mL/mmol) was added 1 eq. of di-tert-butyl azodicarboxylate. The mixture was stirred at 50° C. for the carbamate and at rt for the phenol. After reaching an appropriate conversion the volatiles were removed under reduced pressure, the crude intermediate was purified via flash chromatography using heptane/EtOAc as eluents.

### Mitsunobu General Procedure II

To the mixture of 1.0-1.5 eq. of aliphatic alcohol, 1 eq. of carbamate/phenol, and 1-2 eq. triphenylphosphine in THF or toluene (5 mL/mmol) was added 1-3 eq. of ditertbutyl azodicarboxylate/diisopropyl azodicarboxylate in one portion. The mixture was stirred at rt or 50° C., if necessary, for the carbamate and at rt for the phenol. After reaching an appropriate conversion the volatiles were removed under reduced pressure, the crude intermediate was purified via flash column chromatography.

### Quaternary Salt Deprotection General Procedure

To a THF (5 mL/mmol) solution of the appropriate quaternary salt 3 eq. TBAF was added, and then it was stirred at rt until no further conversion was observed. The reaction mixture was the evaporated to dry under reduced pressure. To a suspension of 1 eq. desilylated quaternary salt in dry MeCN (15 mL/mmol), 100 eq. of HF×Pyr added, and then was stirred at 60° C. After reaching an appropriate conversion, the volatiles were removed under reduced pressure, the residue was suspended in a 1:1 mixture of THF—water (30 mL/mmol), 150 eq. of LiOH×H₂O was added, and the mixture was stirred at rt. After reaching an appropriate conversion, the volatiles were removed under reduced pressure. The crude product was purified via flash chromatography using DCM and MeOH (containing 1.2% NH₃) as eluents.

### Propargylic Amine Preparation General Procedure

An oven-dried vial was equipped with a PTFE-coated magnetic stirring bar, it was charged with 2 eq. PPh₃ and 2 eq. imidazole then DCM (5 mL/mmol) was added. To the resulting mixture 2 eq. iodine was added portionwise then stirred for 15 min at rat. To the resulting mixture 1 eq. of the appropriate alcohol was added dissolved in DCM and stirred at rt until no further conversion was observed. To the generated iodo compound 20 eq. of the appropriate amine was added and then stirred for 30 min at rt, while full conversion was observed. Celite was added to the reaction mixture and the volatiles were removed under reduced pressure. Then it was purified via flash chromatography using DCM and MeOH (1.2% NH₃) eluents.

### Silver Catalyzed Propargylic Amine Preparation General Procedure

A 24 ml vial was equipped with a stirring bar, and charged with 1 eq. of 2-[3-(1,3-benzothiazol-2-ylamino)-4-methyl-6,7-dihydro-5H-pyrido[2,3-c]pyridazin-8-yl]-5-[3-(4-ethynyl-2-fluoro-phenoxy)propyl]thiazole-4-carboxylic acid, 20 eq. paraformaldehyde/acetone and 20 eq. of the appropriate amine were stirred in dry ethanol (5 ml/mmol) in presence of 20 mol % silver tosylate at 80° C. until no further conversion was observed. Celite was added to the reaction mixture and the volatiles were removed under reduced pressure. Then it was purified via flash chromatography using DCM and MeOH (1.2% NH₃) as eluents.

### Hydrolysis General Procedure

The appropriate methyl ester was suspended in a 1:1 mixture of THF—water (5 mL/mmol) and 10 eq. of LiOH×H₂O was added, and the mixture was stirred at 50° C. After reaching an appropriate conversion, the volatiles were removed under reduced pressure; the crude product was purified via flash chromatography using DCM and MeOH (containing 1.2% NH₃) as eluents.

### Amine Substitution and Hydrolysis General Procedure I

To the product from any of the Preparations 12 and 13 in a 1:1 mixture of acetonitrile and N-methyl-2-pyrrolidone (10 ml/mmol), was added the appropriate amine (3-10 eq), and the reaction mixture was stirred at 50° C. for 2-24 h. After the purification of the substitution product by column chromatography (silica gel, using DCM and MeOH as eluents), the product was dissolved in THF (10 ml/mmol), and water (2 ml/mmol) and LiOH×H₂O (3-5 eq) was added. Then, the reaction mixture was stirred at 20-40° C. for 1-4 h. The hydrolysed product was purified by preparative HPLC (using acetonitrile and 5 mM aqueous NH₄HCO₃ solution as eluents) to give the desired product.

### Amine Substitution and Hydrolysis General Procedure II

To the product from Preparation 14_01 in a 1:1 mixture of acetonitrile and N-methyl-2-pyrrolidone (10 ml/mmol), was added the appropriate amine (3-10 eq), and the mixture was stirred at 50° C. for 2-24 h. After the addition of 70% HF in pyridine (50-100 eq) at rt, the mixture was stirred for 4-18 h. After the purification of the substitution product by column chromatography (silica gel, using DCM and MeOH as eluents), the product was dissolved in THF (8 ml/mmol), and water (2 ml/mmol) and LiOH×H₂O (5 eq) was added, and stirred at 20-40° C. for 1-4 h. The hydrolysed product was purified by preparative HPLC (using acetonitrile and 5 mM aqueous NH₄HCO₃ solution as eluents) to give the desired product.

### Amine Substitution and Hydrolysis General Procedure III

To the product from the Preparation 13 or Preparation 16 in acetonitrile (13 ml/mmol) was added the appropriate amine (3 eq) and Na₂CO₃ (12 eq), and the reaction mixture was stirred at 120° C. for 1.5-3 h in a microwave reactor. After the addition of KOH (3 eq), the reaction mixture was stirred at 120° C. for 0.75-1 h. The hydrolysed product was purified by preparative HPLC or HILIC chromatography (using acetonitrile and 5 mM aqueous NH₄HCO₃ solution as eluents) to give the desired product.

### Alkylation, Deprotection and Hydrolysis General Procedure

A mixture of tertiary amine (1 eq.) and alkylating agent (10 eq.) in acetonitrile (3 mL/mmol) was stirred at rt. After reaching appropriate conversion, the volatiles were removed under reduced pressure and purified via reverse phase flash column chromatography, if it was necessary, otherwise the residue was directly dissolved in acetonitrile (3 mL/mmol), HF×Pyr (100 eq.) was added and the mixture was stirred at 60° C. After reaching appropriate conversion, the volatiles were removed under reduced pressure, the residue was suspended in a 1:1 mixture of 1,4-dioxane—water (10 mL/mmol), LiOH×H₂O (150 eq.) was added and the mixture was stirred at 60° C. After reaching appropriate conversion to the desired product, the volatiles were removed under reduced pressure and the crude product was purified via reverse phase flash column chromatography.

### Preparation 1a: Methyl 2-(tert-butoxycarbonylamino)-5-[3-(2-fluoro-4-iodo-phenoxy)propyl]thiazole-4-carboxylate

### Step A: methyl 2-(tert-butoxycarbonylamino)-5-iodo-thiazole-4-carboxylate

50.00 g methyl 2-(tert-butoxycarbonylamino)thiazole-4-carboxylate (193.55 mmol, 1 equiv) was suspended in 600 mL dry MeCN. 52.25 g N-iodo succinimide (232.30 mmol,) was added and the resulting mixture was stirred overnight at room temperature.

The reaction mixture was diluted with saturated brine, then it was extracted with EtOAc. The combined organic layers were extracted with 1 M Na₂S₂O₃, then with brine again. Then dried over Na₂SO₄, filtered and the filtrate was concentrated under reduced pressure. The crude product was purified via flash chromatography using heptane as eluent to obtain 60 g of the desired product (156 mmol, 80% Yield). ¹H NMR (400 MHz, DMSO-d₆): δ ppm 12.03/11.06 (br s), 3.78 (s, 3H), 1.47 (s, 9H); ¹³C NMR (100 MHz, DMSO-d₆) δ ppm 153.8, 82.5, 77.7, 52.3, 28.3; HRMS-ESI (m/z): [M+H]⁺ calculated for C₁₀H₁₄IN₂O₄S: 384.9713; found 384.9708.

### Step B: methyl 2-(tert-butoxycarbonylamino)-5-(3-hydroxyprop-1-ynyl)thiazole-4-carboxylate

A 500 mL oven-dried, one-necked, round-bottom flask was equipped with a PTFE-coated magnetic stirring bar and fitted with a reflux condenser. It was charged with 9.6 g of the product from Step A (25 mmol, 1 equiv), 2.80 g prop-2-yn-1-ol (2.91 mL, 50 mmol, 2 equiv) and 36.10 g DIPA (50 mL, 356.8 mmol, 14.27 equiv) then 125 mL dry THF was added and the system was flushed with argon. After 5 minutes stirring under inert atmosphere 549 mg Pd(PPh₃)₂Cl₂ (1.25 mmol, 0.05 equiv) and 238 mg CuI (1.25 mmol, 0.05 equiv) was added. The resulting mixture was then warmed up to 60° C. and stirred at that temperature until no further conversion was observed. Celite was added to the reaction mixture and the volatiles were removed under reduced pressure. Then it was purified via flash chromatography using heptane and EtOAc as eluents to give 7.30 g of the desired product (23 mmol, 93% Yield) as a yellow solid. ¹H NMR (400 MHz, DMSO-d₆): δ ppm 12.1 (br s, 1H), 5.45 (t, 1H), 4.36 (d, 2H), 3.79 (s, 3H), 1.48 (s, 9H); ¹³C NMR (100 MHz, DMSO-d₆) δ ppm 12.1 (br s, 1H), 5.45 (t, 1H), 4.36 (d, 2H), 3.79 (s, 3H), 1.48 (s, 9H); HRMS-ESI (m/z): [M+H]⁺ calculated for C₁₃H₁₇N₂O₅S: 313.0852, found 313.0866.

### Step C: methyl 2-(tert-butoxycarbonylamino)-5-(3-hydroxypropyl)thiazole-4-carboxylate

An 1 L oven-dried pressure bottle equipped with a PTFE-coated magnetic stir bar was charged with 44.75 g of the product from Step B (143.3 mmol, 1 equiv), 7.62 Pd/C (7.17 mmol, 0.05 equiv) in 340 mL ethanol, and then placed under a nitrogen atmosphere using hydrogenation system. After that, it was filled with 4 bar H₂ gas and stirred at rt overnight. Full conversion was observed, but only the olefin product was formed. After filtration of the catalysts through a pad of Celite, the whole procedure was repeated with 5 mol % new catalysts. The resulting mixtures were stirred overnight to get full conversion. Celite was added to the reaction mixtures and the volatiles were removed under reduced pressure. Then it was purified via flash chromatography column using heptane and EtOAc as eluents to give 31.9 g of the desired product (101 mmol, 70.4% Yield) as light-yellow crystals. ¹H NMR (500 MHz, DMSO-d₆): δ ppm 11.61 (br s, 1H), 4.54 (t, 1H), 3.76 (s, 3H), 3.43 (m, 2H), 3.09 (t, 2H), 1.74 (m, 2H), 1.46 (s, 9H); ¹³C NMR (125 MHz, DMSO-d₆) δ ppm 162.8, 143.1, 135.4, 60.3, 51.9, 34.5, 28.3, 23.4; HRMS-ESI (m/z): [M+H]⁺ calculated for C₁₃H₂₁N₂O₅S: 317.1165, found 317.1164 (M+H).

### Step D: methyl 2-(tert-butoxycarbonylamino)-5-[3-(2-fluoro-4-iodo-phenoxy)propyl]thiazole-4-carboxylate

A 250 mL oven-dried, one-necked, round-bottomed flask equipped with a PTFE-coated magnetic stir bar, was charged with 3.40 g 2-fluoro-4-iodo-phenol (14 mmol, 1 equiv), 5.00 g of the product from Step C (16 mmol, 1.1 equiv) and 4.10 g PPh₃ (16 mmol, 1.1 equiv) dissolved in 71 mL dry toluene. After 5 min stirring under nitrogen atmosphere, 3.10 mL DIAD (3.20 g, 16 mmol, 1.1 equiv) was added in one portion while the reaction mixture warmed up. Then the reaction mixture was heated up to 50° C. and stirred at that temperature for 30 min, when the reaction reached complete conversion. The reaction mixture was directly injected onto a preconditioned silica gel column, and then it was purified via flash chromatography using heptane and EtOAc as eluents. The crude product was crystalized from MeOH to give 4.64 g of the desired product (9.24 mmol, 66% Yield). ¹H NMR (500 MHz, DMSO-d₆) δ ppm 11.64 (br s, 1H), 7.59 (dd, 1H), 7.45 (dd, 1H), 6.98 (t, 1H), 4.06 (t, 2H), 3.73 (s, 3H), 3.22 (t, 2H), 2.06 (m, 2H), 1.46 (s, 9H); ¹³C NMR (125 MHz, DMSO-d₆) δ ppm 134, 124.9, 117.6, 68.2, 51.9, 30.5, 28.3, 23.2; HRMS-ESI (m/z): [M+H]⁺ calculated for C₁₉H₂₃N₂O₅FSI: 537.0350; found 537.0348.

### Preparation 1c: Methyl 2-(tert-butoxycarbonylamino)-5-[3-[4-[3-(dimethylamino)prop-1-ynyl]-2-fluoro-phenoxy]propyl]thiazole-4-carboxylate

A 250 mL oven-dried, one-necked, round-bottom flask was equipped with a PTFE-coated magnetic stirring bar and fitted with a reflux condenser. It was charged with 5.36 g Preparation 1a (10 mmol, 1 equiv), 1.66 g N,N-dimethylprop-2-yn-1-amine (20 mmol, 2 equiv) and 20 mL DIPA (142.7 mmol, 14.27 equiv) then 50 mL dry THF was added and the system was flushed with argon. After 5 minutes stirring under inert atmosphere 220 mg Pd(PPh₃)₂Cl₂ (0.5 mmol, 0.05 equiv) and 95 CuI (0.5 mmol, 0.05 equiv) were added. The resulting mixture was then warmed up to 60° C. and stirred at that temperature until no further conversion was observed. Celite was added to the reaction mixture and the volatiles were removed under reduced pressure. Then it was purified via flash chromatography using DCM and MeOH (1.2% NH₃) as eluents to give 4.5 g of the desired product (7.8 mmol, 78% Yield). ¹H NMR (500 MHz, DMSO-d₆) δ ppm 11.66 (s, 1H), 7.29 (dd, 1H), 7.19 (m, 1H), 7.12 (t, 1H), 4.09 (t, 2H), 3.73 (s, 3H), 3.44 (s, 2H), 3.23 (t, 2H), 2.24 (s, 6H), 2.07 (m, 2H), 1.45 (s, 9H); ¹³C NMR (125 MHz, DMSO-d₆) δ ppm 162.8, 147.3, 129, 119.2, 115.4, 84.3, 68, 51.9, 48.1, 44.2, 30.6, 28.3, 23.2; HRMS-ESI (m/z): [M+H]⁺ calculated for C₂₄H₃₁FN₃O₅S: 492.1962; found 492.1956 (M+H).

### Preparation 2a: 3-(3,6-Dichloro-5-methyl-pyridazin-4-yl)propan-1-ol

### Step A: [(pent-4-yn-1-yloxy)methyl]benzene

To an oven-dried flask was added 4-pentyn-1-ol (11.1 mL, 119 mmol, 1 eq) in THF (100 mL) and the solution was cooled to 0° C. Sodium hydride (60% dispersion; 7.13 g, 178 mmol, 1.5 eq) was added portionwise and the mixture was allowed to stir for 30 min at 0° C. before the dropwise addition of benzyl bromide (15.6 mL, 131 mmol, 1.1 eq). The mixture was allowed to warm to ambient temperature and was stirred for 16 h, then cooled to 0° C., quenched with saturated aqueous ammonium chloride (30 mL) and diluted with water (30 mL). The mixture was extracted with ethyl acetate (2×150 mL), and the combined organic extracts were washed successively with dilute aqueous ammonium hydroxide ammonium hydroxide (150 mL) and brine (100 mL), dried (magnesium sulfate) and concentrated in vacuo. Purification by automated flash column chromatography (CombiFlash Rf, 330 g RediSep™ silica cartridge) eluting with a gradient of 0-10% ethyl acetate in iso-heptane afforded the desired product as a yellow liquid (19.5 g, 112 mmol, 94%). LC/MS (C₁₂H₁₄O) 175 [M+H]⁺; RT 1.28 (LCMS-V-B1). ¹H NMR (400 MHz, Chloroform-d) δ 7.37-7.32 (m, 4H), 7.31-7.27 (m, 1H), 4.52 (s, 2H), 3.58 (t, J=6.1 Hz, 2H), 2.32 (td, J=7.1, 2.6 Hz, 2H), 1.95 (t, J=2.7 Hz, 1H), 1.83 (tt, J=7.1, 6.2 Hz, 2H).

### Step B: [(hex-4-yn-1-yloxy)methyl]benzene

To an oven-dried flask was added the product from Step A (19.5 g, 112 mmol, 1 eq) and tetrahydrofuran (200 mL) and the solution was cooled to −78° C. n-Butyllithium (66.9 mL, 135 mmol, 1.2 eq) was added dropwise over 30 min and the reaction was stirred for 1 h then iodomethane (10.5 mL, 168 mmol, 1.5 eq) was added dropwise and the mixture was allowed to warm to 0° C. over 1 h. The reaction was quenched by the addition of saturated aqueous ammonium chloride (40 mL), diluted with water (40 mL), extracted with ethyl acetate (3×100 mL), and the combined organic extracts were successively washed with 2M aqueous sodium thiosulfate (200 mL) and brine (200 mL), dried (magnesium sulfate) and concentrated in vacuo. Purification by automated flash column chromatography (CombiFlash Rf, 330 g RediSep™ silica cartridge) eluting with a gradient of 0-10% ethyl acetate in iso-heptane afforded the desired product as a yellow liquid (19.2 g, 0.1 mol, 91%). LC/MS (C₁₃H₁₆O) 189 [M+H]⁺; RT 1.34 (LCMS-V-B1). ¹H NMR (400 MHz, DMSO-d₆) δ 7.41-7.23 (m, 5H), 4.46 (s, 2H), 3.48 (t, J=6.3 Hz, 2H), 2.23-2.14 (m, 2H), 1.72 (s, 3H), 1.70-1.65 (m, 2H).

### Step C: 4-[3-(benzyloxy)propyl]-3,6-dichloro-5-methylpyridazine

A solution of 3,6-dichloro-1,2,4,5-tetrazine (5 g, 33.1 mmol, 1 eq) and the product from Step B (7.48 g, 39.8 mmol, 1.2 eq) in tetrahydrofuran (30 mL) was heated at 160° C. for 19 h in a sealed flask. The reaction was allowed to cool to ambient temperature then concentrated in vacuo. Purification by automated flash column chromatography (CombiFlash Rf, 220 g RediSep™ silica cartridge) eluting with a gradient of 0-30% ethyl acetate in iso-heptane afforded the desired product as an orange oil (7.32 g, 23.5 mmol, 71%). LC/MS (C₁₅H₁₆Cl₂N₂O) 311 [M+H]⁺; RT 1.35 (LCMS-V-B1). ¹H NMR (400 MHz, DMSO-d₆) δ 7.45-7.18 (m, 5H), 4.48 (s, 2H), 3.53 (t, J=5.9 Hz, 2H), 2.96-2.83 (m, 2H), 2.42 (s, 3H), 1.88-1.69 (m, 2H).

### Step D: 3-(3,6-dichloro-5-methylpyridazin-4-yl)propan-1-ol

To a cooled solution of the product from Step C (7.32 g, 23.5 mmol, 1 eq) in dichloromethane (100 mL) was added boron trichloride solution (1 M in dichloromethane; 58.8 mL, 58.8 mmol, 2.5 eq) dropwise and the mixture was allowed to stir at ambient temperature for 1 h. The reaction was quenched by the addition of methanol and concentrated in vacuo. The residue was partitioned between dichloromethane (100 mL) and saturated aqueous sodium bicarbonate (150 mL), and the organic phase was washed with brine (150 mL), dried (magnesium sulfate) and concentrated in vacuo. Purification by automated flash column chromatography (CombiFlash Rf, 80 g RediSep™ silica cartridge) eluting with a gradient of 0-80% ethyl acetate in iso-heptane afforded the desired product as a yellow oil (4.19 g, 19 mmol, 81%). LC/MS (C₈H₁₀Cl₂N₂O) 221 [M+H]⁺; RT 0.84 (LCMS-V-B1). ¹H NMR (400 MHz, DMSO-d₆) δ 4.67 (t, J=5.1 Hz, 1H), 3.49 (td, J=6.0, 5.1 Hz, 2H), 2.91-2.80 (m, 2H), 2.43 (s, 3H), 1.72-1.59 (m, 2H).

### Preparation 2ab: 3,6-dichloro-4-(3-iodopropyl)-5-methyl-pyridazine

After stirring PPh₃ (59.3 g, 2 eq), imidazole (15.4 g, 2 eq), and iodine (57.4 g, 2 eq) in 560 mL of DCM for 15 min, 25.0 g of Preparation 2a (113 mmol) was added and stirred for 2 h. The product was purified via flash chromatography using heptane and EtOAc as eluents to give 34.7 g of the desired product (92%). ¹H NMR (500 MHz, DMSO-d₆) δ ppm 3.41 (t, 2H), 2.89 (m, 2H), 2.43 (s, 3H), 1.97 (m, 2H); ¹³C NMR (125 MHz, DMSO-d₆) δ ppm 157.7, 156.8, 141.5, 140.2, 31.4, 31.1, 16.7, 7.8; HRMS (ESI) [M]⁺ calcd for C₈H₉Cl₂IN₂: 330.9266, found 330.9255.

### Preparation 3a: Methyl 2-(3-chloro-4-methyl-6,7-dihydro-5H-pyrido[2,3-c]pyridazin-8-yl)-5-[3-(2-fluoro-4-iodo-phenoxy)propyl]thiazole-4-carboxylate

### Step A: methyl 2-{[(tert-butoxy)carbonyl][3-(3,6-dichloro-5-methylpyridazin-4-yl)propyl]amino}-5-[3-(2-fluoro-4-iodophenoxy)propyl]-1,3-thiazole-4-carboxylate

Using Mitsunobu General Procedure I starting from 4.85 g Preparation 1a (9.04 mmol, 1 equiv) as the appropriate carbamate and 2 g Preparation 2a (9.04 mmol, 1 equiv) as the appropriate alcohol, 4.6 g of the desired product (69% Yield) was obtained. ¹H NMR (500 MHz, DMSO-d₆) δ ppm 7.56 (dd, 1H), 7.44 (dm, 1H), 7.08 (m, 2H), 6.96 (t, 1H), 4.05 (t, 2H), 3.75 (s, 3H), 3.21 (t, 2H), 2.82 (m, 2H), 2.4 (s, 3H), 2.06 (m, 2H), 1.88 (m, 2H), 1.48 (s, 9H); ¹³C NMR (125 MHz, DMSO-d₆) δ ppm 162.7, 157.6, 156.7, 156.5/153.2, 152.2, 147, 142.1, 139.8, 134, 124.9, 117.6, 84, 82.4, 68.1, 52.1, 46.1, 30.4, 28.1, 27.5, 25.8, 23.1, 16.4; HRMS-ESI (m/z): [M+H]⁺ calculated for C₂₇H₃₁Cl₂FIN₄O₅S: 739.0415, found 739.0395.

### Step B: methyl 2-[3-(3,6-dichloro-5-methyl-pyridazin-4-yl)propylamino]-5-[3-(2-fluoro-4-iodo-phenoxy)propyl]thiazole-4-carboxylate

Using Deprotection with HFIPA General Procedure starting from the product from Step A as the appropriate carbamate, 3.70 g the desired product (97% Yield) was obtained. ¹H NMR (500 MHz, DMSO-d₆) δ ppm 7.71 (t, 1H), 7.59 (dd, 1H), 7.44 (dm, 1H), 6.96 (t, 1H), 4.03 (t, 2H), 3.7 (s, 3H), 3.29 (m, 2H), 3.11 (t, 2H), 2.84 (m, 2H), 2.39 (s, 3H), 2 (m, 2H), 1.76 (m, 2H); ¹³C NMR (125 MHz, DMSO-d₆) δ ppm 164.6, 163, 152.3, 147.1, 134.1, 124.8, 117.6, 82.4, 68.1, 51.9, 44, 30.7, 28, 26.9, 23.3, 16.4; HRMS-ESI (m/z): [M+H]⁺ calculated for C₂₂H₂₃Cl₂FIN₄O₃S: 638.9891, found 638.9888.

### Step C: methyl 2-(3-chloro-4-methyl-6,7-dihydro-5H-pyrido[2,3-c]pyridazin-8-yl)-5-[3-(2-fluoro-4-iodo-phenoxy)propyl]thiazole-4-carboxylate

A suspension of 3 g of the product from Step B (4.69 mmol, 1 eq) and 1.81 g cesium carbonate (9.3853 mmol, 2 eq.) were stirred at 80° C. for 3 h in 25 mL dry 1,4-dioxane to reach complete conversion. Reaction mixture directly was evaporated to Celite, and then purified by flash chromatography on using DCM-MeOH as eluents to obtain 2.67 g of the title compound (94% Yield). ¹H NMR (500 MHz, DMSO-d₆) δ ppm 7.57 (dd, 1H), 7.43 (dm, 1H), 6.97 (t, 1H), 4.23 (t, 2H), 4.08 (t, 2H), 3.77 (s, 3H), 3.22 (t, 2H), 2.86 (t, 2H), 2.29 (s, 3H), 2.08 (m, 2H), 2.03 (m, 2H); ¹³C NMR (125 MHz, DMSO-d₆) δ ppm 163.1, 155.4, 152.2, 151.6, 151.2, 147, 142.5, 136, 134.8, 134, 128.9, 124.9, 117.6, 82.3, 68.4, 51.9, 46.3, 30.7, 24.2, 23, 19.7, 15.7; HRMS-ESI (m/z): [M+H]⁺ calculated for C₂₂H₂₂ClFIN₄O₃S: 603.0124, found 603.0108.

### Preparation 3c: 2-[3-(1,3-Benzothiazol-2-ylamino)-4-methyl-6,7-dihydro-5H-pyrido[2,3-c]pyridazin-8-yl]-5-[3-(4-ethynyl-2-fluoro-phenoxy)propyl]thiazole-4-carboxylic acid

### Step A: methyl 2-(3-chloro-4-methyl-6,7-dihydro-5H-pyrido[2,3-c]pyridazin-8-yl)-5-[3-[2-fluoro-4-(2-trimethylsilylethynyl)phenoxy]propyl]thiazole-4-carboxylate

A 250 mL oven-dried, one-necked, round-bottom flask was equipped with a PTFE-coated magnetic stirring bar and fitted with a reflux condenser. It was charged with 5 g Preparation 3a (8.29 mmol, 1 eq.), 2.34 mL ethynyl (trimethyl) silane (16.58 mmol, 2 eq.) and 10 mL DIPEA, then 40 mL dry THF was added and the system was flushed with argon. After 5 minutes stirring under inert atmosphere 182 mg Pd(PPh₃)₂Cl₂ (0.41 mmol, 0.05 eq.) and 79 mg (0.41 mmol, 0.05 eq.) were added. The resulting mixture was then warmed up to 60° C. and stirred at that temperature for 2 hours to reach complete conversion. Celite was added to the reaction mixture and the volatiles were removed under reduced pressure. Then it was purified via flash chromatography using Heptane-EtOAc as eluents to give 4.26 g of the desired product (89% Yield). ¹H NMR (500 MHz, DMSO-d₆) δ ppm 7.31 (dd, 1H), 7.23 (dn, 1H), 7.13 (t, 1H), 4.25 (t, 2H), 4.12 (t, 2H), 3.77 (s, 3H), 3.24 (t, 2H), 2.87 (t, 2H), 2.31 (s, 3H), 2.1 (m, 2H), 2.03 (m, 2H), 0.21 (s, 9H); ¹³C NMR (125 MHz, DMSO-d₆) δ ppm 163.0, 155.3, 151.7, 151.3, 136.1, 129.4, 129.0, 119.4, 115.3, 104.6, 93.7, 68.2, 51.9, 46.3, 30.7, 24.1, 23.0, 19.7, 15.7, 0.4; HRMS-ESI (m/z): [M+H]⁺ calculated for C₂₇H₃₀ClFN₄O₃SSi: 573.1553, found 573.1549.

### Step B: methyl 2-[3-(1,3-benzothiazol-2-ylamino)-4-methyl-6,7-dihydro-5H-pyrido[2,3-c]pyridazin-8-yl]-5-[3-[2-fluoro-4-(2-trimethylsilylethynyl)phenoxy]propyl]thiazole-4-carboxylate

A 100 mL oven-dried, one-necked, round-bottom flask with a PTFE-coated magnetic stirring bar was charged with 4.25 g of the product from Step A (7.4 mmol, 1.0 eq.), 2.23 g 1,3-benzothiazol-2-amine (14.8 mmol, 2.0 eq.) and 3.87 mL DIPEA (2.87 mg, 22.2 mmol, 3.0 eq.) then 40 mL cyclohexanol was added and the system was flushed with argon. After 5 minutes stirring under inert atmosphere 679 mg Pd₂(dba)₃ (0.74 mmol, 0.10 eq.) and 858 mg XantPhos (1.48 mmol, 0.20 eq.) were added. The resulting mixture was then warmed up to 140° C. and stirred at that temperature for 30 min to reach complete conversion. The reaction mixture was diluted with DCM and directly injected onto a preconditioned silica gel column, and then it was purified via flash chromatography using heptane and EtOAc as eluents. The pure fractions were combined and concentrated under reduced pressure to give 3.90 g of the desired product (77% Yield). ¹H NMR (500 MHz, DMSO-d₆) δ ppm 12.27/10.91 (brs, 1H), 8.1-7.1 (brm, 4H), 7.34 (dd, 1H), 7.24 (dm, 1H), 7.16 (t, 1H), 4.25 (t, 2H), 4.15 (t, 2H), 3.78 (s, 3H), 3.28 (t, 2H), 2.87 (t, 2H), 2.34 (s, 3H), 2.13 (m, 2H), 2.04 (m, 2H), 0.19 (s, 9H); HRMS-ESI (m/z): [M+H]⁺ calculated for C₃₄H₃₆FN₆O₃S₂Si: 687.2038, found 687.2020.

### Step C: 2-[3-(1,3-benzothiazol-2-ylamino)-4-methyl-6,7-dihydro-5H-pyrido[2,3-c]pyridazin-8-yl]-5-[3-(4-ethynyl-2-fluoro-phenoxy)propyl]thiazole-4-carboxylic acid

A 10 mL oven-dried, one-necked, round-bottom flask was equipped with a PTFE-coated magnetic stirring bar and fitted with a reflux condenser. It was charged with 343 mg of the product from Step B (0.5 mmol, 1.0 eq.) dissolved in 2.5 mL THF/H₂O (4:1). Then 105 mg LiOH×H₂O (2.50 mmol, 5.0 eq.) was added and the resulting mixture was heated to 60° C. and stirred for 4 h at this temp. The reaction reached complete conversion. Celite gel was added to the reaction mixture and the volatiles were removed under reduced pressure. Then it was purified via flash chromatography using DCM and MeOH (1.2% NH₃) as eluents to give 200 mg title compound (66% Yield). ¹H NMR (500 MHz, DMSO-d₆) δ ppm 7.88 (d, 1H), 7.49 (br., 1H), 7.37 (t, 1H), 7.36 (dd, 1H), 7.25 (dm, 1H), 7.19 (t, 1H), 7.16 (t, 1H), 4.27 (t, 2H), 4.15 (t, 2H), 4.11 (s, 1H), 3.27 (t, 2H), 2.87 (t, 2H), 2.33 (s, 3H), 2.14 (m, 2H), 2.04 (m, 2H); ¹³C NMR (125 MHz, DMSO-d₆) δ ppm 164.2, 151.5, 147.9, 129.4, 126.5, 122.5, 122.3, 119.5, 115.5, 114.5, 82.9, 80.5, 68.5, 46.2, 31.0, 23.9, 23.1, 20.3, 12.9; HRMS-ESI (m/z): [M+H]⁺ calculated for C₃₀H₂₆FN₆O₃S₂: 601.1486, found 601.1498.

### Preparation 3d: Methyl 2-[3-(1,3-benzothiazol-2-ylamino)-4-methyl-6,7-dihydro-5H pyrido[2,3-c]pyridazin-8-yl]-5-[3-[2-fluoro-4-(3-hydroxyprop-1-ynyl)phenoxy]propyl]thiazole-4-carboxylate

### Step A: methyl 5-[3-[4-[3-[tert-butyl(dimethyl)silyl]oxyprop-1-ynyl]-2-fluoro-phenoxy]propyl]-2-(3-chloro-4-methyl-6,7-dihydro-5H-pyrido[2,3-c]pyridazin-8-yl)thiazole-4-carboxylate

Using Sonogashira General Procedure starting from 4.00 g of Preparation 3a (6.63 mmol, 1.0 eq.) and 2.26 g tert-butyl-dimethyl-prop-2-ynoxy-silane (13.27 mmol, 2 eq.) as the appropriate acetylene, 2.80 g of the desired product (65% Yield) was obtained. ¹H NMR (500 MHz, DMSO-d₆) δ ppm 7.27 (dd, 1H), 7.19 (dd, 1H), 7.14 (t, 1H), 4.51 (s, 1H), 4.25 (m, 2H), 4.12 (t, 2H), 3.77 (s, 3H), 3.24 (t, 2H), 2.87 (t, 2H), 2.3 (s, 3H), 2.1 (quint., 2H), 2.03 (m, 2H), 0.88 (s, 9H), 0.12 (s, 6H); ¹³C NMR (125 MHz, DMSO-d₆) δ ppm 163.0, 128.9, 119.1, 115.5, 68.3, 52.1, 51.9, 46.3, 30.7, 26.2, 24.2, 23.0, 19.7, 15.7, −4.6; HRMS-ESI (m/z): [M+H]⁺ calculated for C₃₁H₃₉ClFN₄O₄SSi: 645.2128, found 645.2120.

### Step B: methyl 2-[3-(1,3-benzothiazol-2-ylamino)-4-methyl-6,7-dihydro-5H-pyrido[2,3-c]pyridazin-8-yl]-5-[3-[4-[3-[tert-butyl(dimethyl)silyl]oxyprop-1-ynyl]-2-fluoro-phenoxy]propyl]thiazole-4-carboxylate

Using Buchwald General Procedure II starting from 2.8 g of the product from Step A (4.34 mmol, 1.0 eq.) and 1.30 g 1,3-benzothiazol-2-amine (8.67 mmol, 2.0 eq.), 2.1 g of the desired product (64% Yield) was obtained. ¹H NMR (500 MHz, DMSO-d₆) δ ppm 12.25/10.91 (brs 1H), 7.88 (br, 1H), 7.51 (br, 1H), 7.37 (t, 1H), 7.29 (dd, 1H), 7.2 (t, 1H), 7.2 (dd, 1H), 7.17 (t, 1H), 4.49 (s, 2H), 4.25 (t, 2H), 4.14 (t, 2H), 3.77 (s, 3H), 3.27 (t, 2H), 2.86 (t, 2H), 2.32 (s, 3H), 2.13 (qn, 2H), 2.04 (qn, 2H), 0.87 (s, 9H), 0.1 (s, 6H); ¹³C NMR (125 MHz, DMSO-d₆) δ ppm 163.2, 155.7, 151.6, 148.5, 147.6, 141.5, 128.9, 127.6, 126.5, 122.5, 122.3, 119.1, 116.9, 115.5, 114.8, 88.2, 84, 68.4, 52.1, 51.9, 46.4, 31, 26.2, 24, 23.1, 20.4, 12.9, −4.6; HRMS-ESI (m/z): [M+H]⁺ calculated for C₃₈H₄₄FN₆O₄S₂Si: 759.2613, found 759.2609.

### Step C: methyl 2-[3-(1,3-benzothiazol-2-ylamino)-4-methyl-6,7-dihydro-5H-pyrido[2,3-c]pyridazin-8-yl]-5-[3-[2-fluoro-4-(3-hydroxyprop-1-ynyl)phenoxy]propyl]thiazole-4-carboxylate

A 100 mL oven-dried, one-necked, round-bottom flask was equipped with a PTFE-coated magnetic stirring bar and fitted with a reflux condenser. It was charged with 2.10 g of the product from Step B (2.76 mmol, 1.0 eq.) dissolved in 15 mL THF. Then 3.32 mL TBAF (3.32 mmol, 1.2 eq., 1 M in THF) was added dropwise via syringe over a period of 2 minutes, and stirred at that temperature for 30 min. The reaction mixture was quenched with saturated NH₄Cl, then directly evaporated to Celite and it was purified via flash chromatography using heptane-EtOAc as eluents to give 1.6 g of the desired product (90% Yield). ¹H NMR (500 MHz, DMSO-d₆) δ ppm 11.14 (brs, 1H), 7.83 (brd, 1H), 7.49 (brs, 1H), 7.36 (m, 1H), 7.24 (dd, 1H), 7.19 (m, 1H), 7.18 (dm, 1H), 7.15 (t, 1H), 5.08 (t, 1H), 4.28 (m, 2H), 4.27 (d, 2H), 4.17 (t, 2H), 3.8 (s, 3H), 3.29 (m, 2H), 2.89 (m, 2H), 2.35 (s, 3H), 2.15 (m, 2H), 2.07 (m, 2H); HRMS-ESI (m/z): [M+H]⁺ calculated for C₃₂H₃₀FN₆O₄S₂: 645.1748, found 645.1738.

### Preparation 3f: Ethyl 2-{3-[(1,3-benzothiazol-2-yl)amino]-4-methyl-5H,6H,7H,8H-pyrido[2,3-c]pyridazin-8-yl}-1,3-thiazole-4-carboxylate

### Step A: ethyl 2-[(hex-4-yn-1-yl)amino]-1,3-thiazole-4-carboxylate

To a solution of ethyl 2-bromo-1,3-thiazole-4-carboxylate (1.17 g, 4.97 mmol, 1 eq) in acetonitrile (16 mL) was added hex-4-yn-1-amine (725 mg, 7.46 mmol, 1.5 eq) and triethylamine (1.04 mL, 7.46 mmol, 1.5 eq) and the mixture was heated at 150° C. for 4 h under microwave irradiation. The reaction was partitioned between ethyl acetate and brine, and the organic phase was dried (magnesium sulfate) and concentrated in vacuo. Purification by automated flash column chromatography (CombiFlash Rf, 40 g RediSep™ silica cartridge) eluting with a gradient of 0-60% ethyl acetate in iso-heptane afforded the desired product as a beige solid (741 mg, 2.94 mmol, 59%). LC/MS (C₁₂H₁₆N₂O₂S) 253 [M+H]⁺; RT 2.32 (LCMS-V-C).

### Step B: ethyl 2-{3-chloro-4-methyl-5H,6H,7H,8H-pyrido[2,3-c]pyridazin-8-yl}-1,3-thiazole-4-carboxylate

To a solution of 3,6-dichloro-1,2,4,5-tetrazine (443 mg, 2.94 mmol, 1 eq) in tetrahydrofuran (15 mL) was added the product from Step A (741 mg, 2.94 mmol, 1 eq) and the mixture was heated in a sealed tube at 110° C. overnight. The reaction was concentrated in vacuo and the residue was triturated with methanol, filtered and dried under vacuum to afford the desired product as a beige solid (607 mg, 1.79 mmol, 61%). LC/MS (C₁₄H₁₅ClN₄O₂S) 339 [M+H]⁺; RT 2.41 (LCMS-V-C). ¹H NMR (400 MHz, DMSO-d₆) δ 8.06 (s, 1H), 4.38-4.25 (m, 4H), 2.92 (t, J=6.3 Hz, 2H), 2.34 (s, 3H), 2.14-2.01 (m, 2H), 1.31 (t, J=7.1 Hz, 3H).

### Step C: ethyl 2-{3-[(1,3-benzothiazol-2-yl)amino]-4-methyl-5H,6H, 7H,8H-pyrido[2,3-c]pyridazin-8-yl}-1,3-thiazole-4-carboxylate

To an oven-dried microwave vial was added the product from Step B (607 mg, 1.79 mmol, 1 eq), 2-aminobenzothiazole (404 mg, 2.69 mmol, 1.5 eq)), XantPhos (207 mg, 0.36 mmol, 0.2 eq), cesium carbonate (1.17 g, 3.58 mmol, 2 eq) and 1,4-dioxane (36 mL) and the vessel was evacuated and flushed with nitrogen then tris(dibenzylideneacetone)dipalladium(0) (164 mg, 0.18 mmol, 0.1 eq) was added and the mixture was sparged with nitrogen (10 mins) then heated at 150° C. for 4 hours under microwave irradiation. The reaction was diluted with ethyl acetate and filtered through celite, then washed with brine, dried (magnesium sulfate) and concentrated in vacuo. Purification by automated flash column chromatography (CombiFlash R_{f}, 24 g RediSep™ silica cartridge) eluting with a gradient of 0-100% ethyl acetate in iso-heptane afforded a solid that was triturated with diethyl ether, filtered and dried under vacuum to afford the desired product as a yellow solid (329 mg, 0.73 mmol, 41%). LC/MS (C₂₁H₂ON₆O₂S₂) 453 [M+H]⁺; RT 2.73 (LCMS-V-C). ¹H NMR (400 MHz, DMSO-d₆) δ 7.99 (br s+s, 2H), 7.65 (br s, 1H), 7.43-7.31 (m, 1H), 7.28-7.15 (m, 1H), 4.35-4.25 (m, 4H), 2.96-2.85 (m, 2H), 2.36 (s, 3H), 2.15-2.00 (m, 2H), 1.32 (t, J=7.1 Hz, 3H).

### Preparation 3g: Ethyl 5-(3-hydroxypropyl)-2-(4-methyl-3-{[(2Z)-3-{[2-(trimethylsilyl)ethoxy]methyl}-2,3-dihydro-1,3-benzothiazol-2-ylidene]amino}-5H,6H,7H,8H-pyrido[2,3-c]pyridazin-8-yl)-1,3-thiazole-4-carboxylate

### Step A: ethyl 2-(4-methyl-3-{[(2Z)-3-{[2-(trimethylsilyl)ethoxy]methyl}-2,3-dihydro-1,3-benzothiazol-2-ylidene]amino}-5H,6H,7H,8H-pyrido[2,3-c]pyridazin-8-yl)-1,3-thiazole-4-carboxylate

To a solution of the product from Preparation 3f (11.7 g, 25.8 mmol, 1 eq) in dimethylformamide (700 mL) was added N,N-diisopropylethylamine (13.5 mL, 77.4 mmol, 3 eq). After 5 min the mixture was cooled to 0° C. and 4-(dimethylamino)pyridine (630 mg, 5.16 mmol, 0.2 eq) and 2-(trimethylsilyl)ethoxymethyl chloride (13.6 mL, 77.4 mmol, 3 eq) were added and the mixture was stirred at ambient temperature overnight. The reaction was concentrated in vacuo, then partitioned between dichloromethane and brine, and the organic phase was dried (magnesium sulfate) and concentrated in vacuo. Purification by automated flash column chromatography (CombiFlash Rf, 330 g RediSep™ silica cartridge) eluting with a gradient of 0-40% ethyl acetate in iso-heptane afforded the desired product as a yellow solid (9.61 g, 16.5 mmol, 64%). LC/MS (C₂₇H₃₄N₆O₃SiS₂) 583 [M+H]⁺; RT 2.90 (LCMS-V-C). ¹H NMR (400 MHz, DMSO-d₆) δ 7.99 (s, 1H), 7.82 (dd, J=7.7, 1.1 Hz, 1H), 7.49-7.38 (m, 2H), 7.28-7.19 (m, 1H), 5.86 (s, 2H), 4.38-4.23 (m, 4H), 3.77-3.67 (m, 2H), 2.89 (t, J=6.2 Hz, 2H), 2.38 (s, 3H), 2.13-2.01 (m, 2H), 1.31 (t, J=7.1 Hz, 3H), 0.91 (dd, J=8.5, 7.4 Hz, 2H), −0.11 (s, 9H).

### Step B: ethyl 5-bromo-2-(4-methyl-3-{[(2Z)—3-{[2-(trimethylsilyl)ethoxy]methyl}-2,3-dihydro-1,3-benzothiazol-2-ylidene]amino}-5H,6H,7H,8H-pyrido[2,3-c]pyridazin-8-yl)-1,3-thiazole-4-carboxylate

To a solution of the product of Step A (9.61 g, 16.5 mmol, 1 eq) in dichloromethane (400 mL) was added N-bromosuccinimide (3.52 g, 19.8 mmol, 1.2 eq) and the mixture was stirred at ambient temperature overnight. The reaction was partitioned between dichloromethane and water, and the organic phase was washed with brine, dried (PTFE phase separator) and concentrated in vacuo. Purification by automated flash column chromatography (CombiFlash Rf, 220 g RediSep™ silica cartridge) eluting with a gradient of 0-40% ethyl acetate in iso-heptane afforded the desired product as a yellow solid (9.66 g, 14.6 mmol, 89%). LC/MS (C₂₇H₃₃BrN₆O₃SiS₂) 663 [M+H]⁺; RT 3.13 (LCMS-V-C). ¹H NMR (400 MHz, DMSO-d₆) δ 7.84 (dd, J=7.5, 1.1 Hz, 1H), 7.59-7.38 (m, 2H), 7.24 (ddd, J=8.3, 6.7, 1.7 Hz, 1H), 5.85 (s, 2H), 4.37-4.23 (m, 4H), 3.72 (dd, J=8.5, 7.4 Hz, 2H), 2.87 (t, J=6.2 Hz, 2H), 2.38 (s, 3H), 2.13-2.00 (m, 2H), 1.32 (t, 3H), 0.95-0.81 (m, 2H), −0.12 (s, 9H).

### Step C: ethyl 5-[(1E)-3-[(tert-butyldimethylsilyl)oxy]prop-1-en-1-yl]-2-(4-methyl-3-{[(2Z)-3-{[2-(trimethylsilyl)ethoxy]methyl}-2,3-dihydro-1,3-benzothiazol-2-ylidene]amino}-5H,6H,7H,8H-pyrido[2,3-c]pyridazin-8-yl)-1,3-thiazole-4-carboxylate

To an oven-dried sealed flask was added the product from Step B (9.66 g, 14.6 mmol, 1 eq), (E)-3-(tert-butyldimethylsilyloxy)propene-1-yl-boronic acid pinacol ester (5.74 mL, 17.5 mmol, 1.2 eq), potassium carbonate (6.05 g, 43.8 mmol, 3 eq), [1,1′-bis(diphenylphosphino)ferrocene]dichloropalladium(II) (1.19 g, 1.46 mmol, 0.1 eq), tetrahydrofuran (360 mL) and water (120 mL), and the mixture was sparged with nitrogen (10 min) then heated at 120° C. for 2 h. The reaction was partitioned between ethyl acetate and water, and the organic layer was washed with brine, dried (magnesium sulfate) and concentrated in vacuo. Purification by automated flash column chromatography (CombiFlash Rf, 220 g RediSep™ silica cartridge) eluting with a gradient of 0-30% ethyl acetate in iso-heptane afforded the desired product as a yellow solid (6.46 g, 8.58 mmol, 59%). LC/MS (C₃₆H₅₂N₆O₄Si₂S₂) 753 [M+H]⁺; RT 1.62 (LCMS-V-B2). ¹H NMR (400 MHz, DMSO-d₆) δ 7.80 (dd, J=7.6, 1.0 Hz, 1H), 7.51-7.38 (m, 3H), 7.24 (ddd, J=8.3, 6.8, 1.8 Hz, 1H), 6.28 (dt, J=16.0, 4.3 Hz, 1H), 5.85 (s, 2H), 4.37 (dd, J=4.4, 2.1 Hz, 2H), 4.35-4.25 (m, 4H), 3.72 (dd, J=8.5, 7.4 Hz, 2H), 2.88 (t, J=6.3 Hz, 2H), 2.37 (s, 3H), 2.09-1.99 (m, 2H), 1.31 (t, J=7.1 Hz, 3H), 0.93 (s, 9H), 0.92-0.83 (m, 2H), 0.11 ((s, 6H), −0.11 (s, 9H).

### Step D: ethyl 5-{3-[(tert-butyldimethylsilyl)oxy]propyl}-2-(4-methyl-3-{[(2Z)-3-{[2-(trimethylsilyl)ethoxy]methyl}-2,3-dihydro-1,3-benzothiazol-2-ylidene]amino}-5H,6H,7H,8H-pyrido[2,3-c]pyridazin-8-yl)-1,3-thiazole-4-carboxylate

To a solution of the product from Step C (6.46 g, 8.58 mmol, 1 eq) in ethyl acetate (300 mL) was added platinum (IV) oxide (390 mg, 1.72 mmol, 0.2 eq) under a nitrogen atmosphere. The vessel was evacuated and backfilled with nitrogen (×3), then evacuated, placed under an atmosphere of hydrogen, and shaken for 3 days at ambient temperature. The reaction was filtered through celite, eluted with ethyl acetate and concentrated in vacuo to afford the desired product as a brown gum (6.72 g, 8.9 mmol, >100%). LC/MS (C₃₆H₅₄N₆O₄Si₂S₂) 755 [M+H]⁺; RT 1.67 (LCMS-V-B2). ¹H NMR (400 MHz, DMSO-d₆) δ 7.76 (d, 1H), 7.48-7.35 (m, 2H), 7.24 (ddd, J=8.2, 6.5, 1.9 Hz, 1H), 5.84 (s, 2H), 4.33-4.22 (m, 4H), 3.76-3.62 (m, 4H), 3.15 (t, J=7.5 Hz, 2H), 2.87 (t, J=6.4 Hz, 2H), 2.37 (s, 3H), 2.10-1.98 (m, 3H), 1.91-1.79 (m, 2H), 1.31 (t, J=7.1 Hz, 3H), 0.95-0.85 (m, 11H), 0.06 (s, 6H), −0.12 (s, 9H).

### Step E: ethyl 5-(3-hydroxypropyl)-2-(4-methyl-3-{[(2Z)—3-{[2-(trimethylsilyl)ethoxy]methyl}-2,3-dihydro-1,3-benzothiazol-2-ylidene]amino}-5H,6H,7H,8H-pyrido[2,3-c]pyridazin-8-yl)-1,3-thiazole-4-carboxylate

To a solution of the product from Step D (6.72 g, 8.9 mmol, 1 eq) in 1,4-dioxane (400 mL) was added hydrochloric acid (4M in dioxane; 67 mL, 267 mmol, 30 eq) and the mixture was stirred at ambient temperature for 1 h. The reaction cooled to 0° C. and neutralised with 1N aqueous sodium hydroxide (300 mL), then partitioned between ethyl acetate and water, and the organic phase was dried (magnesium sulfate) and concentrated in vacuo. Purification by automated flash column chromatography (CombiFlash Rf, 120 g RediSep™ silica cartridge) eluting with a gradient of 0-80% ethyl acetate in iso-heptane gave a solid that was triturated with diethyl ether, filtered and dried under vacuum to afford the desired product as a white solid (3.87 g, 6.04 mmol, 68%). LC/MS (C₃₀H₄₀N₆O₄SiS₂) 641 [M+H]⁺; RT 2.80 (LCMS-V-C). ¹H NMR (400 MHz, DMSO-d₆) δ 7.83 (dd, J=7.6, 1.1 Hz, 1H), 7.48-7.37 (m, 2H), 7.23 (ddd, J=8.3, 6.7, 1.8 Hz, 1H), 5.85 (s, 2H), 4.56 (t, J=5.1 Hz, 1H), 4.33-4.22 (m, 4H), 3.72 (dd, J=8.6, 7.3 Hz, 2H), 3.48 (td, J=6.3, 5.1 Hz, 2H), 3.17-3.08 (m, 2H), 2.88 (t, J=6.4 Hz, 2H), 2.38 (s, 3H), 2.11-1.99 (m, 2H), 1.87-1.75 (m, 2H), 1.31 (t, J=7.1 Hz, 3H), 0.96-0.86 (m, 2H), −0.11 (s, 9H).

### Preparation 4c: tert-Butyl N-[3-(3-fluoro-4-hydroxy-phenyl)prop-2-ynyl]-N-methyl-carbamate

Using Sonogashira General Procedure starting from 10.00 g of 2-fluoro-4-iodo-phenol (42.0 mmol, 1 eq.) as the appropriate phenol and 10.67 g of tert-butyl N-methyl-N-prop-2-ynyl-carbamate (63.1 mmol, 1.5 eq.) as the alkyne, 10.8 g (92%) of the desired product was obtained. ¹H NMR (500 MHz, DMSO-d₆) δ ppm 10.32 (s, 1H), 7.22 (brd, 1H), 7.08 (dm, 1H), 6.92 (dd, 1H), 4.21 (s, 2H), 2.85 (s, 3H), 1.41 (s, 9H); ¹³C NMR (125 MHz, DMSO-d₆) δ ppm 150.8, 146.4, 129.0, 119.6, 118.4, 113.2, 84.4, 82.7, 38.5, 33.8, 28.5; HRMS-ESI (m/z): [M−C₄H₈+H]⁺ calculated for C₁₁H₁₁FNO₃: 224.0717, found 224.0720.

### Preparation 7: tert-butyl-diphenyl-[2-[[3,5-dimethyl-7-[[5-methyl-4-(4,4,5,5-tetramethyl-1,3,2-dioxaborolan-2-yl)pyrazol-1-yl]methyl]-1-adamantyl]oxy]ethoxy]silane

### Step A: 3-bromo-5,7-dimethyladamantane-1-carboxylic acid

After stirring iron (6.7 g, 120 mmol) in bromine (30.7 mL, 600 mmol, 5 eq) at 0° C. for 1 h, 3,5-dimethyladamantane-1-carboxylic acid (25 g, 1 eq) was added and the reaction mixture was stirred at rt for 2 days. After the addition of EtOAc, the reaction mixture was treated carefully with a saturated solution of sodium-thiosulfate at 0° C. and stirred for 15 min. After filtration through a pad of Celite and rinsing with EtOAc, the organic phase was separated, washed with a saturated solution of sodium-thiosulfate and brine, dried, concentrated to give the desired product (34.28 g, 74.6%), which was used without further purification. ¹H NMR (400 MHz, DMSO-d₆): δ ppm 12.33 (br., 1H), 2.21 (s, 2H), 1.96/1.91 (d+d, 4H), 1.50/1.43 (d+d, 4H), 1.21/1.14 (dm+dm, 2H), 0.86 (s, 6H); ¹³C NMR (100 MHz, DMSO-d₆) δ ppm 176.8, 66.8, 54.0, 48.7, 48.5, 45.7, 43.3, 35.5, 29.4; HRMS-ESI (m/z): [M−H]− calculated for C₁₃H₁₈BrO₂: 285.0496; found 285.0498.

### Step B: 3-bromo-5,7-dimethyl-1-adamantyl-methanol

To the product from Step A (34.3 g, 119 mmol) in THF (77.6 mL) was added slowly a 1 M solution of BH3-THF in THF (358 mL, 3 eq) and the reaction mixture was stirred for 18 h. After the addition of methanol and stirring for 30 min, purification by column chromatography (silica gel, heptane and MTBE as eluents) afforded the desired product (16.19 g, 49.6%). ¹H NMR (400 MHz, DMSO-d₆): δ ppm 4.51 (t, 1H), 3.05 (d, 2H), 1.91 (s, 2H), 1.91 (s, 4H), 1.19/1.09 (d+d, 2H), 1.19/1.05 (d+d, 4H), 0.85 (s, 6H) ¹³C NMR (100 MHz, DMSO-d₆) δ ppm 70.4, 68.9, 54.9, 49.8, 49.3, 43.8, 41.4, 35.7, 29.7; HRMS-ESI (m/z): [M−Br]− calculated for C₁₃H₂₁O: 193.1598 found: 193.1589.

### Step C: 1-[3-bromo-5,7-dimethyl-1-adamantyl]methyl]pyrazole

To the product from Step B (16.19 g, 59.26 mmol) and 1H-pyrazole (4.841 g, 1.2 eq) in toluene (178 mL) was added cyanomethylenetributylphosphorane (18.64 mL, 1.2 eq) in one portion and the reaction mixture was stirred at 90° C. for 2 h. Purification by column chromatography (silica gel, heptane and MTBE as eluents) afforded the desired product (17.88 g, 93%). ¹H NMR (400 MHz, DMSO-d₆): δ ppm 7.63 (d, 1H), 7.43 (d, 1H), 6.23 (t, 1H), 3.90 (s, 2H), 1.92-1.02 (m, 12H), 0.83 (s, 6H); ¹³C NMR (100 MHz, DMSO-d₆) δ ppm 139.0, 131.8, 105.2, 67.7, 61.4, 54.4/48.8/44.6, 50.4, 35.7, 29.6; HRMS-ESI (m/z): [M]⁺ calculated for C₁₆H₂₃BrN₂: 322.1045 found: 322.1014.

### Step D: 5-methyl-1-[[-3-bromo-5,7-dimethyl-1-adamantyl]methyl]pyrazole

To the solution of the product from Step C (17.88 g, 55.3 mmol) in THF (277 mL) was added butyllithium (2.5 M in THF, 66 mL, 3 eq) at −78° C., then after 1 h, iodomethane (17.2 mL, 5 eq) was added. After 10 min, the reaction mixture was quenched with a saturated solution of NH₄Cl, extracted with EtOAc and the combined organic layers were dried and concentrated to give the desired product (18.7 g, 100%), which was used in the next step without further purification. ¹H NMR (400 MHz, DMSO-d₆): δ ppm 7.31 (d, 1H), 6.00 (d, 1H), 3.79 (s, 2H), 2.23 (s, 3H), 2.01 (s, 2H), 1.89/1.85 (d+d, 4H), 1.23/1.15 (d+d, 4H), 1.16/1.05 (d+d, 2H), 0.83 (s, 6H); ¹³C NMR (100 MHz, DMSO-d₆) δ ppm 139.2, 138.0, 105.2, 67.8, 57.8, 54.4, 50.6, 48.8, 44.8, 41.5, 35.7, 29.6, 11.8; HRMS-ESI (m/z): [M+H]⁺ calculated for C₁₇H₂₆BrN₂: 337.1279 found: 337.1289.

### Step E: 2-[[-3,5-dimethyl-7-[(5-methylpyrazol-1-yl)methyl]-1-adamantyl]oxy]ethanol

The mixture of the product from Step D (18.7 g, 55.3 mmol), ethylene glycol (123 mL, 40 eq), and DIPEA (48.2 mL, 5 eq) was stirred at 120° C. for 6 h. After the reaction mixture was diluted with water and extracted with EtOAc, the combined organic layers were dried and concentrated to give the desired product (18.5 g, 105%), which was used in the next step without further purification. ¹H NMR (400 MHz, DMSO-d₆): δ ppm 7.29 (d, 1H), 5.99 (d, 1H), 4.45 (t, 1H), 3.78 (s, 2H), 3.39 (q, 2H), 3.32 (t, 2H), 2.23 (s, 3H), 1.34 (s, 2H), 1.27/1.21 (d+d, 4H), 1.13/1.07 (d+d, 4H), 1.04/0.97 (d+d, 2H), 0.84 (s, 6H); ¹³C NMR (100 MHz, DMSO-d₆) δ ppm 139.0, 137.8, 105.1, 74.0, 62.1, 61.5, 58.5, 50.1, 47.0, 46.1, 43.3, 39.7, 33.5, 30.2, 11.9; HRMS-ESI (m/z): [M+H]⁺ calculated for C₁₉H₃₁N₂O₂: 319.2386 found: 319.2387.

### Step F: tert-butyl-diphenyl-[2-[[-3,5-dimethyl-7-[(5-methylpyrazol-1-yl)methyl]-1-adamantyl]oxy]ethoxy]silane

To the mixture of the product from Step E (17.6 g, 55.3 mmol) and imidazole (5.65 g, 1.5 eq) in DCM (150 ml) was added tert-butyl-chloro-diphenyl-silane (18.6 g, 1.2 eq) and the reaction mixture was stirred for 1 h. Purification by column chromatography (silica gel, heptane and MTBE as eluents) afforded the desired product (27.0 g, 87.8%). ¹H NMR (400 MHz, DMSO-d₆): δ ppm 7.72-7.34 (m, 10H), 7.29 (d, 1H), 5.99 (br., 1H), 3.78 (s, 2H), 3.67 (t, 2H), 3.44 (t, 2H), 2.21 (s, 3H), 1.33 (s, 2H), 1.26/1.18 (d+d, 4H), 1.12/1.06 (d+d, 4H), 1.03/0.96 (d+d, 2H), 0.98 (s, 9H), 0.82 (s, 6H); ¹³C NMR (100 MHz, DMSO-d₆) δ ppm 139.0, 137.8, 105.1, 74.2, 64.4, 61.7, 58.5, 50.0, 46.9, 46.0, 43.4, 39.6, 33.5, 30.1, 27.1, 19.3, 11.9; HRMS-ESI (m/z): [M+H]⁺ calculated for C₃₅H₄₉N₂O₂Si: 557.3563 found: 557.3564.

### Step G: tert-butyl-diphenyl-[2-[[3-[(4-iodo-5-methyl-pyrazol-1-yl)methyl]-5,7-dimethyl-1-adamantyl]oxy]ethoxy]silane

To the solution of the product from Step F (27.0 g, 48.56 mmol) in DMF (243 mL) was added N-iodosuccinimide (13.6 g, 1.25 eq) and the reaction mixture was stirred for 2 h. After the dilution with water, the mixture was extracted with DCM. The combined organic layers were washed with saturated solution of sodium-thiosulphate and brine, dried, and concentrated to afford the desired product (30.1 g, 90%). ¹H NMR (400 MHz, DMSO-d₆): δ ppm 7.68-7.37 (m, 10H), 7.45 (s, 1H), 3.89 (s, 2H), 3.67 (t, 2H), 3.44 (t, 2H), 2.23 (s, 3H), 1.30 (s, 2H), 1.26/1.17 (d+d, 4H), 1.12/1.05 (d+d, 4H), 1.00/0.96 (d+d, 2H), 0.98 (s, 9H), 0.82 (s, 6H); ¹³C NMR (100 MHz, DMSO-d₆) δ ppm 142.5, 140.8, 133.7, 64.4, 61.7, 60.3, 59.9, 49.9, 46.8, 45.9, 43.2, 39.7, 33.5, 30.1, 27.1, 19.3, 12.2; HRMS-ESI (m/z): [M+H]⁺ calculated for C₃₅H₄₈IN₂O₂Si: 683.2530 found: 683.2533.

### Step H: tert-butyl-diphenyl-[2-[[3,5-dimethyl-7-[[5-methyl-4-(4,4,5,5-tetramethyl-1,3,2-dioxaborolan-2-yl)pyrazol-1-yl]methyl]-1-adamantyl]oxy]ethoxy]silane

To the product from Step G (17.5 g, 25.6 mmol) in THF (128 mL) was added chloro(isopropyl)magnesium-LiCl (1.3 M in THF, 24 mL, 1.2 eq) at 0° C., stirred for 40 min, treated with 2-isopropoxy-4,4,5,5-tetramethyl-1,3,2-dioxaborolane (15.7 mL, 3 eq), and the reaction mixture was stirred for 10 min. After dilution with a saturated solution NH₄Cl and extraction with EtOAc, the combined organic phases were concentrated and was purified by column chromatography (silica gel, heptane and MTBE as eluents) to give the desired product (15.2 g, 86.9%). ¹H NMR (400 MHz, DMSO-d₆): δ ppm 7.65 (dm, 4H), 7.47 (s, 1H), 7.45 (tm, 2H), 7.40 (tm, 4H), 3.80 (s, 2H), 3.66 (t, 2H), 3.44 (t, 2H), 2.35 (s, 3H), 1.35-0.94 (m, 12H), 1.24 (s, 12H), 0.97 (s, 9H), 0.83 (s, 6H); ¹³C NMR (100 MHz, DMSO-d₆) δ ppm 146.9, 144.3, 135.6, 130.2, 128.2, 104.7, 83.0, 74.2, 64.4, 61.7, 58.4, 30.1, 27.1, 25.2, 19.3, 12.0; HRMS-ESI (m/z): [M+H]⁺ calculated for C₄₁H₆₀BN₂O₄Si: 683.4415 found: 683.4423.

### Preparation 8: tert-butyl-[3-[3,5-dimethyl-7-[[5-methyl-4-(4,4,5,5-tetramethyl-1,3,2-dioxaborolan-2-yl)pyrazol-1-yl]methyl]-1-adamantyl]propoxy]-diphenyl-silane

### Step A: 1-[[3-allyl-5,7-dimethyl-1-adamantyl]methyl]-5-methyl-pyrazole

To the product of Step D of Preparation 7 (15.66 g, 46.43 mmol) and AgOTf (597 mg, 0.05 eq) in THF (232 mL) was added a 2 M solution of allyl-Mg—Cl in THF (46.4 mL, 2 eq) and the reaction mixture was stirred for 0.5 h. After quenching with a saturated solution of NH₄Cl and extracting with EtOAc, the combined organic phases were concentrated and purified by column chromatography (silica gel, heptane and MTBE as eluents) to give the desired product (11.32 g, 81.7%). ¹H NMR (400 MHz, DMSO-d₆): δ ppm 7.27 (d, 1H), 5.98 (m, 1H), 5.76 (m, 1H), 5.01/4.96 (dm+dm, 2H), 3.73 (s, 2H), 2.22 (s, 3H), 1.83 (d, 2H), 1.15-0.93 (m, 12H), 0.78 (s, 6H); ¹³C NMR (100 MHz, DMSO-d₆) δ ppm 139.0, 137.7, 135.0, 117.7, 105.0, 59.0, 47.8, 44.2, 35.0, 31.8, 30.6, 11.9; HRMS-ESI (m/z): [M+H]⁺ calculated for C₂₀H₃₁N₂: 299.2487 found: 299.2485.

### Step B: 3-[3,5-dimethyl-7-[(5-methylpyrazol-1-yl)methyl]-1-adamantyl]propan-1-ol

To the product of Step A (10.2 g, 34.17 mmol), in THF (85 mL) was added a 1 M solution of BH3-THF in THF (85.4 mL, 2 eq) and the reaction mixture was stirred for 1 h. After treatment with a 10 M solution of NaOH (24 mL, 7 eq) and a 33% solution of hydrogen peroxide (73 mL, 25 eq) at 0° C., the reaction was stirred at rt for 1 h. Then, it was quenched with aqueous HCl solution, extracted with EtOAc, and purified by column chromatography (silica gel, heptane and MTBE as eluents) to give the desired product (9.75 g, 90%). ¹H NMR (400 MHz, DMSO-d₆): δ ppm 7.28 (d, 1H), 5.98 (m, 1H), 4.33 (t, 1H), 3.73 (s, 2H), 3.32 (m, 2H), 2.22 (brs, 3H), 1.32 (m, 2H), 1.12-0.92 (m, 12H), 1.06 (m, 2H), 0.78 (s, 6H); ¹³C NMR (100 MHz, DMSO-d₆) δ ppm 137.7, 105.0, 62.1, 59.1, 39.7, 30.7, 26.5, 11.9, HRMS-ESI (m/z): [M+H]⁺ calculated for C₂₀H₃₃N₂O: 317.2593 found: 317.2590

### Step C: tert-butyl-[3-[3,5-dimethyl-7-[(5-methylpyrazol-1-yl)methyl]-1-adamantyl]propoxy]-diphenyl-silane

To the product of Step B (9.75 g, 30.8 mmol) and imidazole (3.1 g, 1.5 eq) in DCM (92 ml) was added tert-butyl-chloro-diphenyl-silane (9.45 mL, 1.2 eq) and the reaction mixture was stirred for 1 h. Purification by column chromatography (silica gel, heptane and MTBE as eluents) afforded the desired product (12.5 g, 73%). ¹H NMR (400 MHz, DMSO-d₆): δ ppm 7.63-7.39 (m, 10H), 7.27 (d, 1H), 5.98 (d, 1H), 3.72 (s, 2H), 3.59 (t, 2H), 2.21 (s, 3H), 1.42 (m, 2H), 1.1-0.92 (br., 12H), 1.09 (m, 2H), 0.98 (s, 9H), 0.77 (s, 6H); ¹³C NMR (100 MHz, DMSO-d₆) δ ppm 137.7, 105.0, 64.8, 59.1, 39.3, 38.0, 34.2, 31.8, 30.6, 27.2, 26.1, 19.2, 11.9; HRMS-ESI (m/z): [M+H]⁺ calculated for C₃₆H₅₁N₂OSi: 555.3771 found: 555.3770.

### Step D: tert-butyl-[3-[3-[(4-iodo-5-methyl-pyrazol-1-yl)methyl]-5,7-dimethyl-1-adamantyl]propoxy]-diphenyl-silane

To the product of Step C (12.5 g, 22.54 mmol) in DMF (112 mL) was added N-iodosuccinimide (6.34 g, 1.25 eq) and the reaction mixture was stirred for 2 h. After quenching with a saturated solution of sodium thiosulfate and extraction with DCM, the combined organic phases were washed with saturated sodium thiosulphate and brine, dried, and evaporated to afford the desired product (16.3 g, 105%). LC/MS (C₃₆H₅₀IN₂OSi) 681 [M+H]⁺.

### Step E: tert-butyl-[3-[3,5-dimethyl-7-[[5-methyl-4-(4,4,5,5-tetramethyl-1,3,2-dioxaborolan-2-yl)pyrazol-1-yl]methyl]-1-adamantyl]propoxy]-diphenyl-silane

To the product of Step D (16.25 g, 23.9 mmol) in THF (119 mL) was added chloro(isopropyl)magnesium-LiCl (1.3 M in THF, 22 mL, 1.2 eq.) at 0° C., the mixture was stirred for 40 min, treated with 2-isopropoxy-4,4,5,5-tetramethyl-1,3,2-dioxaborolane (14.6 mL, 3 eq), and stirred for 10 min. After dilution with a saturated solution NH₄Cl and extraction with EtOAc, the combined organic phases were concentrated and was purified by column chromatography (silica gel, heptane and MTBE as eluents) to give the desired product (11.4 g, 70%). ¹H NMR (400 MHz, DMSO-d₆): δ ppm 7.59 (d, 4H), 7.46 (s, 1H), 7.45 (t, 2H), 7.43 (t, 4H), 3.74 (s, 2H), 3.59 (t, 2H), 2.35 (s, 3H), 1.41 (qn, 2H), 1.24 (s, 12H), 1.09 (m, 2H), 1.08 (s, 4H), 1.05 (s, 2H), 0.98 (s, 9H), 0.98 (s, 2H), 0.94 (s, 4H), 0.78 (s, 6H); ¹³C NMR (100 MHz, DMSO-d₆) δ ppm 146.9, 144.2, 135.5, 133.8, 130.3, 128.3, 104.6, 83.0, 64.7, 64.7, 59.0, 50.6, 48.2, 46.5, 44.1, 39.2, 37.9, 31.8, 30.7, 27.2, 26.1, 25.2, 19.2, 12.0; HRMS-ESI (m/z): [M+H]⁺ calculated for C₄₂H₆₂BN₂O₃Si: 681.4623 found: 681.4631.

### Preparation 10: methyl 3-bromo-6-[3-(3,6-dichloro-5-methyl-pyridazin-4-I)propylamino]pyridine-2-carboxylate

### Step A: methyl 6-[bis(tert-butoxycarbonyl)amino]-3-bromo-pyridine-2-carboxylate

To methyl 6-amino-3-bromo-pyridine-2-carboxylate (25.0 g, 108.2 mmol) and DMAP (1.3 g, 0.1 eq) in DCM (541 mL) was added Boc2O (59.0 g, 2.5 eq) at 0° C. and the reaction mixture was stirred for 2.5 h. After the addition of a saturated solution of NaHCO₃ and extraction with DCM, the combined organic phases were dried and concentrated to afford the desired product (45.0 g, 72.3%). LC/MS (C₁₇H₂₃BrN₂O₆Na) 453 [M+Na]⁺.

### Step B: methyl 3-bromo-6-(tert-butoxycarbonylamino)pyridine-2-carboxylate

To the product from Step A (42.7 g, 74.34 mmol) in DCM (370 mL) was added TFA (17.1 mL, 3 eq) at 0° C. and the reaction mixture was stirred for 18 h. After washing with a saturated solution of NaHCO₃ and brine, the combined organic phases were dried, concentrated, and purified by column chromatography (silica gel, heptane and EtOAc as eluents) to give the desired product (28.3 g, 115.2%). ¹H NMR (400 MHz, DMSO-d₆): δ ppm 10.29 (s, 1H), 8.11 (d, 1H), 7.88 (d, 1H), 3.87 (s, 3H), 1.46 (s, 9H) ¹³C NMR (100 MHz, DMSO-d₆) δ ppm 165.6, 153.1, 151.8/148.3, 143.5, 116.3, 109.2, 53.2, 28.4. LC/MS (C₁₂H₁₅BrN₂O₄Na) 353 [M+Na]⁺.

### Step C: methyl 3-bromo-6-[tert-butoxycarbonyl-[3-(3,6-dichloro-5-methyl-pyridazin-4-yl)propyl]amino]pyridine-2-carboxylate

To the product from Step B (10.0 g, 30.1967 mmol) in acetone (150 mL), were added Cs₂CO₃ (29.5 g, 3 eq) and 3,6-dichloro-4-(3-iodopropyl)-5-methyl-pyridazine (Preparation 2ab, 9.9 g, 1 eq) and the reaction mixture was stirred for 18 h. After dilution with water and extraction with EtOAc, the combined organic phases were washed with brine, dried and concentrated to give the desired product (17.5 g, 108%). ¹H NMR (400 MHz, DMSO-d₆): δ ppm 8.13 (d, 1H), 7.78 (d, 1H), 3.91 (t, 2H), 3.89 (s, 3H), 2.79 (m, 2H), 2.38 (s, 3H), 1.82 (m, 2H), 1.46 (s, 9H); ¹³C NMR (100 MHz, DMSO-d₆) δ ppm 165.3, 157.6, 156.6, 153.2, 152.9, 147.2, 143.1, 142.2, 139.7, 122.6, 111.8, 82.2, 53.3, 46.4, 28.1, 27.7, 26.5, 16.3; HRMS-ESI (m/z): [M+Na]⁺ calculated for C₂₀H₂₃BrCl₂N₄NaO₄: 555.0177 found: 555.0172.

### Step D: methyl 3-bromo-6-[3-(3,6-dichloro-5-methyl-pyridazin-4-I)propylamino]pyridine-2-carboxylate

The product from Step C (17.5 g, 32.7 mmol) in 1,1,1,3,3,3-hexafluoroisopropanol (330 mL) was stirred at 110° C. for 18 h. Purification by column chromatography (silica gel, heptane and EtOAc as eluents) afforded the desired product (9.9 g, 70%). ¹H NMR (400 MHz, DMSO-d₆): δ ppm 7.63 (d, 1H), 7.22 (t, 1H), 6.57 (d, 1H), 3.83 (s, 3H), 3.30 (m, 2H), 2.83 (m, 2H), 2.37 (s, 3H), 1.74 (m, 2H) ¹³C NMR (100 MHz, DMSO-d₆) δ ppm 166.5, 141.5, 112.6, 52.9, 40.9, 28.0, 27.0, 16.4.

### Preparation 11: (4-methoxyphenyl)methyl 3-bromo-6-[3-(3,6-dichloro-5-methyl-pyridazin-4-yl)propylamino]pyridine-2-carboxylate

### Step A: 3-bromo-6-[3-(3,6-dichloro-5-methyl-pyridazin-4-yl)propylamino]pyridine-2-carboxylic acid

The mixture of the product from Preparation 10 (35.39 g, 81.52 mmol) and LiOH×H₂O (13.68 g, 4 eq) in 1,4-dioxane (408 mL) and water (82 mL) was stirred at 60° C. for 1 h. After quenching with a 1 M solution of HCl and extraction with EtOAc, the combined organic phases were dried, concentrated, and purified by flash chromatography (silica gel, using DCM and MeOH as eluents) to give the desired product (27.74 g, 81%). LC/MS (C₁₄H₁₄BrCl₂N₄O₂) 421 [M+H]⁺.

### Step B: (4-methoxyphenyl)methyl 3-bromo-6-[3-(3,6-dichloro-5-methyl-pyridazin-4-yl)propylamino]pyridine-2-carboxylate

To the product of Step A (27.7 g, 65.9 mmol), (4-methoxyphenyl)methanol (16.4 mL, 2 eq), and PPh₃ (34.6 g, 2 eq) in toluene (660 mL) and THF (20 ml) was added dropwise diisopropyl azodicarboxylate (26 mL, 2 eq) and the reaction mixture was stirred at 50° C. for 1 h. Purificationby flash chromatography (silica gel, using heptane and EtOAc as eluents) afforded the desired product (23.65 g, 66.4%). ¹H NMR (500 MHz, dmso-d₆) δ ppm 7.62 (d, 1H), 7.37 (dn, 2H), 7.21 (t, 1H), 6.91 (dm, 2H), 6.56 (d, 1H), 5.25 (s, 2H), 3.74 (s, 3H), 3.30 (q, 2H), 2.81 (m, 2H), 2.33 (s, 3H), 1.73 (m, 2H); ¹³C NMR (500 MHz, dmso-d₆) δ ppm 165.9, 159.7, 157.6, 157.5, 156.8, 148.0, 142.7, 141.5, 139.7, 130.6, 127.8, 114.3, 112.6, 101.6, 67.0, 55.6, 40.9, 28.0, 27.1, 16.4; HRMS-ESI (m/z): [M+H]⁺ calculated for C₂₂H₂₂BrCl₂N₄O₃: 539.0252, found: 539.0246.

### Preparation 12: methyl 6-[3-(1,3-benzothiazol-2-ylamino)-4-methyl-6,7-dihydro-5H pyrido[2,3-c]pyridazin-8-yl]-3-[1-[[3,5-dimethyl-7-[2-(p-tolylsulfonyloxy)ethoxy]-1-adamantyl]methyl]-5-methyl-pyrazol-4-yl]pyridine-2-carboxylate

### Step A: methyl 6-[3-(3,6-dichloro-5-methyl-pyridazin-4-yl)propylamino]-3-[5-methyl-1-[[3-[2-[tert-butyl(diphenyl)silyl]oxyethoxy]-5,7-dimethyl-1-adamantyl]methyl]pyrazol-4-yl]pyridine-2-carboxylate

The mixture of the product from Preparation 10 (15.0 g, 34.55 mmol), the product from Preparation 7 (30.7 g, 1.3 eq), Cs₂CO₃ (33.8 g, 3.0 eq), and Pd(AtaPhos)₂Cl₂ (1.53 g, 0.1 eq) in 1,4-dioxane (207 mL) and H₂O (34.5 mL) was stirred at 80° C. for 1.5 h. Purification by column chromatography (silica gel, heptane and EtOAc as eluents) afforded the desired product (18.5 g, 58%). ¹H NMR (400 MHz, DMSO-d₆): δ ppm 7.69-7.37 (m, 10H), 7.32 (d, 1H), 7.23 (s, 1H), 6.98 (t, 1H), 6.63 (d, 1H), 3.82 (s, 2H), 3.67 (t, 2H), 3.58 (s, 3H), 3.46 (t, 2H), 3.35 (m, 2H), 2.86 (m, 2H), 2.40 (s, 3H), 2.06 (s, 3H), 1.78 (m, 2H), 1.35 (s, 2H), 1.27/1.2 (m+m, 4H), 1.15/1.09 (m+m, 4H), 1.05/0.97 (m+m, 2H), 0.97 (s, 9H), 0.84 (s, 6H); HRMS-ESI (m/z): [M+H]⁺ calculated for C₅₀H₆₅Cl₂N₆O₄Si: 909.4057 found: 909.4053.

### Step B: methyl 6-(3-chloro-4-methyl-6,7-dihydro-5H-pyrido[2,3-c]pyridazin-8-yl)-3-[5-methyl-1-[[3-[2-[tert-butyl(diphenyl)silyl]oxyethoxy]-5,7-dimethyl-1-adamantyl]methyl]pyrazol-4-yl]pyridine-2-carboxylate

The mixture of the product from Step A (18.5 g, 20.3 mmol), Cs₂CO₃ (13.2 g, 2 eq), DIPEA (7.1 mL, 2 eq), and Pd(Ataphos)₂Cl₂ (900 mg, 0.1 eq) in 1,4-dioxane (102 mL) was stirred at 110° C. for 18 h. After filtration and concentration, the residue was taken up with DCM, washed with water, and purified by column chromatography (silica gel, DCM and EtOAc as eluents) to give the desired product (12.6 g, 71%). ¹H NMR (400 MHz, DMSO-d₆): δ ppm 7.85 (d, 1H), 7.69 (d, 1H), 7.66 (dm, 4H), 7.47-7.36 (m, 6H), 7.38 (s, 1H), 3.97 (t, 2H), 3.87 (s, 2H), 3.68 (t, 2H), 3.66 (s, 3H), 3.47 (t, 2H), 2.87 (t, 2H), 2.30 (s, 3H), 2.14 (s, 3H), 1.99 (br., 2H), 1.38 (s, 2H), 1.32-0.96 (br., 10H), 0.98 (s, 9H), 0.85 (s, 6H); ¹³C NMR (100 MHz, DMSO-d₆) δ ppm 139.9, 137.6, 120.5, 64.4, 61.7, 58.9, 52.3, 46.0, 43.4, 30.2, 27.1, 24.6, 21.0, 15.5, 10.9; HRMS-ESI (m/z): [M+H]⁺ calculated for C₅₀H₆₂ClN₆O₄Si: 873.4290 found: 873.4291.

### Step C: methyl 6-(3-chloro-4-methyl-6,7-dihydro-5H-pyrido[2,3-c]pyridazin-8-yl)-3-[1-[[3-(2-hydroxyethoxy)-5,7-dimethyl-1-adamantyl]methyl]-5-methyl-pyrazol-4-yl]pyridine-2-carboxylate

To the product from Step B (8.46 g, 9.68 mmol) in THF (95 mL) was added a 1 M solution of TBAF in THF (10.6 mL, 1.1 eq) at 0° C. and the reaction mixture was stirred for 2 h. After quenching with a saturated solution of NH₄Cl and extraction with EtOAc, the combined organic phases were washed with brine, dried, and purified by column chromatography (silica gel, DCM and MeOH as eluents) to give the desired product (5.38 g, 88%). ¹H NMR (400 MHz, DMSO-d₆): δ ppm 7.86 (d, 1H), 7.71 (d, 1H), 7.38 (s, 1H), 4.46 (t, 1H), 3.97 (t, 2H), 3.87 (s, 2H), 3.70 (s, 3H), 3.40 (m, 2H), 3.35 (t, 2H), 2.87 (t, 2H), 2.30 (s, 3H), 2.15 (s, 3H), 1.99 (m, 2H), 1.42-0.95 (m, 12H), 0.87 (s, 6H); HRMS-ESI (m/z): [M+H]⁺ calculated for C₃₄H₄₄ClN₆O: 635.3113 found: 635.3112.

### Step D: methyl 6-[3-(1,3-benzothiazol-2-ylamino)-4-methyl-6,7-dihydro-5H-pyrido[2,3-c]pyridazin-8-yl]-3-[1-[[3-(2-hydroxyethoxy)-5,7-dimethyl-1-adamantyl]methyl]-5-methyl-pyrazol-4-yl]pyridine-2-carboxylate

Using Buchwald General Procedure I at 130° C. for 1 h, starting from 3.7 g of the product from Step C (5.78 mmol) and 1.74 g of 1,3-benzothiazol-2-amine (2 eq), 3.1 g of the desired product (72% Yield) were obtained. ¹H NMR (400 MHz, DMSO-d₆): δ ppm 7.96 (d, 1H), 7.82 (br., 1H), 7.70 (d, 1H), 7.50 (br., 1H), 7.38 (s, 1H), 7.35 (t, 1H), 7.17 (t, 1H), 4.46 (br., 1H), 4.00 (t, 2H), 3.88 (s, 2H), 3.70 (s, 3H), 3.40 (brt., 2H), 3.35 (t, 2H), 2.86 (t, 2H), 2.32 (s, 3H), 2.16 (s, 3H), 2.03-1.94 (m, 2H), 1.42-0.96 (m, 12H), 0.87 (s, 6H); ¹³C NMR (100 MHz, DMSO-d₆) δ ppm 139.8, 137.5, 126.4, 122.4, 122.1, 119.0, 62.1, 61.5, 59.0, 52.6, 45.4, 30.2, 24.3, 21.7, 12.6, 10.9; HRMS-ESI (m/z): [M+H]⁺ calculated for C₄₁H₄₉N₈O₄S: 749.3597 found: 749.3595.

### Step E: methyl 6-[3-(1,3-benzothiazol-2-ylamino)-4-methyl-6,7-dihydro-5H-pyrido[2,3-c]pyridazin-8-yl]-3-[1-[[3,5-dimethyl-7-[2-(p-tolylsulfonyloxy)ethoxy]-1-adamantyl]methyl]-5-methyl-pyrazol-4-yl]pyridine-2-carboxylate

To the product from Step D (3.85 g, 5.14 mmol) and triethylamine (2.15 mL, 3 eq) in DCM (50 mL) was added p-tolylsulfonyl 4-methylbenzenesulfonate (2.51 g, 1.5 eq) and the reaction mixture was stirred for 1 h. Purification by column chromatography (silica gel, heptane and EtOAc as eluents) afforded the desired product (3.2 g, 69%). ¹H NMR (400 MHz, DMSO-d₆): δ ppm 7.96 (d, 1H), 7.81 (br., 1H), 7.77 (d, 2H), 7.70 (d, 1H), 7.50 (br., 1H), 7.46 (d, 2H), 7.39 (s, 1H), 7.35 (t, 1H), 7.17 (t, 1H), 4.06 (t, 2H), 4.00 (t, 2H), 3.85 (s, 2H), 3.69 (s, 3H), 3.49 (t, 2H), 2.86 (t, 2H), 2.40 (s, 3H), 2.32 (s, 3H), 2.15 (s, 3H), 1.99 (m, 2H), 1.32-0.93 (m, 12H), 0.84 (s, 6H); ¹³C NMR (100 MHz, DMSO-d₆) δ ppm 139.8, 137.6, 130.6, 128.1, 126.4, 122.4, 122.1, 119, 71.5, 58.8, 58.4, 52.6, 45.4, 30.1, 24.3, 21.7, 21.6, 12.6, 10.9; HRMS-ESI (m/z): [M+H]⁺ calculated for C₄₈H₅₅N₈O₆S₂: 903.3686 found: 903.3685.

### Preparation 13: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-[3-(p-tolylsulfonyloxy)propyl]-1-adamantyl]methyl]-5-methyl-pyrazol-4-yl]pyridine-2-carboxylate

### Step A: (4-methoxyphenyl)methyl 3-[1-[[3-[3-[tert-butyl(diphenyl)silyl]oxypropyl]-5,7-dimethyl-1-adamantyl]methyl]-5-methyl-pyrazol-4-yl]-6-[3-(3,6-dichloro-5-methyl-pyridazin-4-yl)propylamino]pyridine-2-carboxylate

The mixture of the product from Preparation 11 (3.67 g, 6.79 mmol), the product from Preparation 8 (5.09 g, 1.1 eq), Pd(AtaPhos)₂Cl₂ (301 mg, 0.1 eq), and Cs₂CO₃ (6.64 g, 3 eq) in 1,4-dioxane (41 mL) and H₂O (6.8 mL) was stirred at 80° C. for 18 h. Purification by column chromatography (silica gel, heptane and EtOAc as eluents) afforded the desired product (4.43 g, 64%). ¹H NMR (400 MHz, DMSO-d₆): δ ppm 7.62-7.38 (m, 10H), 7.32 (d, 1H), 7.26 (s, 1H), 7.10 (m, 2H), 6.98 (t, 1H), 6.83 (m, 2H), 6.63 (d, 1H), 4.98 (s, 2H), 3.74 (s, 2H), 3.70 (s, 3H), 3.58 (t, 2H), 3.35 (m, 2H), 2.84 (m, 2H), 2.34 (s, 3H), 2.02 (s, 3H), 1.77 (m, 2H), 1.43 (m, 2H), 1.18-0.85 (m, 12H), 1.09 (t, 2H), 0.97 (s, 9H), 0.77 (s, 6H); HRMS-ESI (m/z): [M+H]⁺ calculated for C₅₈H₂₁Cl₂N₆O₄Si: 1013.4683 found: 1013.4683;

### Step B: (4-methoxyphenyl)methyl 3-[1-[[3-[3-[tert-butyl(diphenyl)silyl]oxypropyl]-5,7-dimethyl-1-adamantyl]methyl]-5-methyl-pyrazol-4-yl]-6-(3-chloro-4-methyl-6,7-dihydro-5H-pyrido[2,3-c]pyridazin-8-yl)pyridine-2-carboxylate

The mixture of the product from Step A (4.43 g, 4.37 mmol), Cs₂CO₃ (2.84 g, 2 eq), DIPEA (1.5 mL, 2 eq) and Pd(Ataphos)₂Cl₂ (193 mg, 0.1 eq) in 1,4-dioxane (22 mL) was stirred at 110° C. for 18 h. After quenching with water and extracting with EtOAc, the combined organic phases were dried, concentrated, and purified by column chromatography (silica gel, DCM and EtOAc as eluents) to give the desired product (2.83 g, 66%). ¹H NMR (400 MHz, DMSO-d₆): δ ppm 7.84 (d, 1H), 7.68 (d, 1H), 7.59 (d, 4H), 7.44 (t, 2H), 7.42 (t, 4H), 7.38 (s, 1H), 7.14 (d, 2H), 6.87 (d, 2H), 5.07 (s, 2H), 3.96 (t, 2H), 3.78 (s, 2H), 3.71 (s, 3H), 3.59 (t, 2H), 2.86 (t, 2H), 2.29 (s, 3H), 2.08 (s, 3H), 1.97 (qn, 2H), 1.43 (qn, 2H), 1.12 (s, 4H), 1.10 (s, 2H), 1.09 (t, 2H), 0.97 (s, 9H), 0.95 (s, 2H), 0.94/0.91 (d+d, 4H), 0.78 (s, 6H); ¹³C NMR (100 MHz, DMSO-d₆) δ ppm 166.9, 159.6, 156.3, 153.6, 150.8, 147.7, 140.1, 137.5, 137.3, 136.0, 135.5, 133.8, 130.3, 130.1, 129.1, 128.3, 127.6, 123.1, 120.5, 115.5, 114.3, 66.8, 64.8, 64.8, 59.6, 55.6, 50.5, 48.1, 46.4, 46.0, 44.2, 39.3, 38.1, 31.7, 30.6, 27.2, 26.1, 24.6, 21.0, 19.3, 15.5, 10.9; HRMS-ESI (m/z): [M+H]⁺ calculated for C₅₈H₇₀ClN₆O₄Si: 977.4916 found: 977.4915.

### Step C: (4-methoxyphenyl)methyl 6-(3-chloro-4-methyl-6,7-dihydro-5H-pyrido[2,3-c]pyridazin-8-yl)-3-[1-[[3-(3-hydroxypropyl)-5,7-dimethyl-1-adamantyl]methyl]-5-methyl-pyrazol-4-yl]pyridine-2-carboxylate

To the product from Step B (2.83 g, 2.89 mmol) in THF (95 mL) was added a 1 M solution of TBAF in THF (3.2 mL, 1.1 eq) at 0° C. and the reaction mixture was stirred for 2 h. After quenching with a saturated solution of NH₄Cl and extracted with EtOAc, the combined organic phases were washed with brine, dried, concentrated, and purified by column chromatography (silica gel, DCM and MeOH as eluents) to give the desired product (2.21 g, 103%). ¹H NMR (400 MHz, DMSO-d₆): δ ppm 7.85 (d, 1H), 7.70 (d, 1H), 7.39 (s, 1H), 7.17 (d, 2H), 6.90 (d, 2H), 5.09 (s, 2H), 4.34 (t, 1H), 3.96 (t, 2H), 3.79 (s, 2H), 3.74 (s, 3H), 3.32 (q, 2H), 2.86 (t, 2H), 2.29 (s, 3H), 2.09 (s, 3H), 1.98 (qn, 2H), 1.34 (qn, 2H), 1.13 (s, 2H), 1.13 (s, 4H), 1.06 (t, 2H), 0.99/0.95 (d+d, 4H), 0.97 (s, 2H), 0.78 (s, 6H); ¹³C NMR (100 MHz, DMSO-d₆) δ ppm 166.9, 159.7, 156.4, 153.6, 150.8, 147.7, 140.2, 137.5, 137.3, 136.0, 130.2, 129.1, 127.6, 123.1, 120.4, 115.5, 114.3, 66.8, 66.8, 62.1, 59.7, 55.6, 50.6, 48.2, 46.5, 46.0, 44.3, 39.7, 38.1, 31.8, 30.6, 26.5, 24.6, 21.0, 15.5, 10.9; HRMS-ESI (m/z): [M+H]⁺ calculated for C₄₂H₅₂ClN₆O₄: 739.3739 found: 739.3739.

### Step D: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-(3-hydroxypropyl)-5,7-dimethyl-1-adamantyl]methyl]-5-methyl-pyrazol-4-yl]pyridine-2-carboxylate

The mixture of the product from Step C (1.71 g, 2.31 mmol), 1,3-benzothiazol-2-amine (695 mg, 2 eq), Pd₂dba₃ (212 mg, 0.1 eq), XantPhos (268 mg, 0.2 eq), and DIPEA (1.2 mL, 3 eq) in cyclohexanol (14 mL) was stirred at 130° C. for 1 h. Purification by column chromatography (silica gel, heptane, DCM and MeCN as eluents) afforded the desired product (1.25 g, 63%). ¹H NMR (400 MHz, DMSO-d₆): δ ppm 12.08/10.87 (brs/brs, 1H), 7.95 (d, 1H), 7.81 (br, 1H), 7.68 (d, 1H), 7.50 (br, 1H), 7.39 (s, 1H), 7.35 (t, 1H), 7.18 (d, 2H), 7.17 (t, 1H), 6.90 (d, 2H), 5.10 (s, 2H), 4.34 (t, 1H), 3.99 (t, 2H), 3.79 (s, 2H), 3.74 (s, 3H), 3.33 (q, 2H), 2.85 (t, 2H), 2.32 (s, 3H), 2.11 (s, 3H), 1.98 (qn, 2H), 1.34 (qn, 2H), 1.14 (s, 4H), 1.14 (s, 2H), 1.07 (t, 2H), 1.00/0.95 (d+d, 2H), 0.99/0.95 (d+d, 4H), 0.79 (s, 6H); ¹³C NMR (100 MHz, DMSO-d₆) δ ppm 140.0, 137.6, 130.2, 126.4, 122.4, 122.0, 119.0, 114.3, 66.7, 62.1, 59.6, 55.6, 50.6, 48.2, 46.5, 45.4, 44.3, 39.7, 30.6, 26.5, 24.3, 21.7, 12.6, 11.0; HRMS-ESI (m/z): [M+H]⁺ calculated for C₄₉H₅₇N₈O₄S: 853.4223 found: 853.4229.

### Step E: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-[3-(p-tolylsulfonyloxy)propyl]-1-adamantyl]methyl]-5-methyl-pyrazol-4-yl]pyridine-2-carboxylate

To the product from Step D (1.25 g, 1.47 mmol) and triethylamine (0.61 mL, 3 eq) in DCM (15 mL) was added p-tolylsulfonyl 4-methylbenzenesulfonate (717 mg, 1.5 eq) and the reaction mixture was stirred for 1 h. Purification by column chromatography (silica gel, heptane and EtOAc as eluents) afforded 800 mg (54%) of the desired product. ¹H NMR (400 MHz, DMSO-d₆): δ ppm 7.95 (d, 1H), 7.88 (brs, 1H), 7.77 (m, 2H), 7.68 (d, 1H), 7.62 (brs, 1H), 7.47 (m, 2H), 7.39 (s, 1H), 7.35 (brs, 1H), 7.17 (brs, 1H), 7.10 (m, 2H), 6.90 (m, 2H), 5.09 (s, 2H), 4.00 (m, 2H), 3.98 (t, 2H), 3.77 (s, 2H), 3.74 (s, 3H), 2.85 (t, 2H), 2.40 (s, 3H), 2.32 (s, 3H), 2.09 (s, 3H), 1.98 (m, 2H), 1.45 (m, 2H), 1.17-0.8 (m, 12H), 0.98 (m, 2H), 0.77 (s, 6H); HRMS-ESI (m/z): [M+H]⁺ calculated for C₅₆H₆₃N₈O₆S₂: 1007.4312 found: 1007.4318.

### Preparation 15: ethyl 2-(3-chloro-4-methyl-6,7-dihydro-5H-pyrido[2,3-c]pyridazin-8-yl)-5-[3-(2-fluoro-4-iodo-phenoxy)propyl]thiazole-4-carboxylate

### Step A: 2-(3-chloro-4-methyl-6,7-dihydro-5H-pyrido[2,3-c]pyridazin-8-yl)-5-[3-(2-fluoro-4-iodo-phenoxy)propyl]thiazole-4-carboxylic acid

The mixture of the product from Preparation 3a (35.39 g, 81.52 mmol) and LiOH×H₂O (4 eq) in 1,4-dioxane (408 mL) and water (82 mL) was stirred at 60° C. for 1 h. After quenching with a 1 M solution of HCl and extraction with EtOAc, the combined organic phases were dried, concentrated, and purified by flash chromatography (silica gel, using DCM and MeOH as eluents) to give the desired product (27.7 g, 81%). ¹H NMR (500 MHz, dmso-d₆) δ ppm 7.56 (dd, 1H), 7.43 (brd., 1H), 6.96 (t, 1H), 4.18 (t, 2H), 4.05 (t, 2H), 3.28 (t, 2H), 2.84 (t, 2H), 2.29 (s, 3H), 2.07 (m, 2H), 1.97 (m, 2H); ¹³C NMR (500 MHz, dmso-d₆) δ ppm 166.4, 154.8, 152.1, 151.8, 151.1, 147.1, 143.9, 135.7, 134.0, 133.8, 129.0, 124.9, 117.6, 82.3, 68.8, 46.3, 31.0, 24.0, 22.5, 19.8, 15.7; HRMS-ESI (m/z): [M+H]⁺ calculated for C₂₁H₂₀ClFIN₄O₃S: 588.9973 found: 588.9969.

### Step B: ethyl 2-(3-chloro-4-methyl-6,7-dihydro-5H-pyrido[2,3-c]pyridazin-8-yl)-5-[3-(2-fluoro-4-iodo-phenoxy)propyl]thiazole-4-carboxylate

To the mixture of the product of Step A (27.7 g, 65.9 mmol), ethanol (2 eq) and PPh₃ (2 eq) in toluene (660 mL) and THF (20 ml) was added dropwise diisopropyl azodicarboxylate (2 eq) and the reaction was stirred at 50° C. 1 h. Purification by flash chromatography (silica gel, using heptane and EtOAc as eluents) afforded the desired product (23.65 g, 66.4%). ¹H NMR (500 MHz, dmso-d₆) δ ppm 7.59 (dd, 1H), 7.44 (dm, 1H), 6.98 (t, 1H), 4.29 (m, 2H), 4.25 (q, 2H), 4.08 (t, 2H), 3.24 (t, 2H), 2.89 (t, 2H), 2.32 (s, 3H), 2.09 (m, 2H), 2.04 (m, 2H), 1.28 (t, 3H); ¹³C NMR (500 MHz, dmso-d₆) δ ppm 162.6, 155.4, 152.2, 151.7, 151.3, 147.0, 134.0, 124.9, 117.6, 82.4, 68.3, 60.7, 46.3, 30.8, 24.1, 23.1, 19.7, 15.7, 14.6; HRMS-ESI (m/z): [M+H]⁺ calculated for C₂₃H₂₄ClFIN₄O₃S: 617.0286, found: 617.0282.

### Preparation 16: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-[2-(p-tolylsulfonyloxy)ethoxy]-1-adamantyl]methyl]-5-methyl-pyrazol-4-yl]pyridine-2-carboxylate

### Step A: 3-[1-[[3-[2-[tert-butyl(diphenyl)silyl]oxyethoxy]-5,7-dimethyl-1-adamantyl]methyl]-5-methyl-pyrazol-4-yl]-6-(3-chloro-4-methyl-6,7-dihydro-5H-pyrido[2,3-c]pyridazin-8-yl)pyridine-2-carboxylic acid

The mixture of 1.5 g (1.72 mmol) of the product of Preparation 12, Step B, 290 mg (4 eq) of LiOH in 17 mL of a 4:1 mixture of THF and water was stirred at 60° C. to reach complete conversion. After the reaction was quenched by the addition of 1M aqueous HCl solution, the mixture was extracted with EtOAc and the organic phases were dried, concentrated, and purified by column chromatography (silica gel, using DCM and MeOH as eluents) to give 1.23 g (83%) of the desired product. ¹H NMR (500 MHz, dmso-d₆) δ ppm 13.11 (s, 1H), 7.80 (d, 1H), 7.66 (d, 4H), 7.65 (d, 1H), 7.44 (t, 2H), 7.41 (s, 1H), 7.40 (t, 4H), 3.99 (t, 2H), 3.86 (s, 2H), 3.68 (t, 2H), 3.47 (t, 2H), 2.87 (t, 2H), 2.29 (s, 3H), 2.17 (s, 3H), 1.99 (qn, 2H), 1.39 (s, 2H), 1.27/1.22 (d+d, 4H), 1.17/1.12 (d+d, 4H), 1.05/0.99 (d+d, 2H), 0.98 (s, 9H), 0.85 (s, 6H); ¹³C NMR (500 MHz, dmso-d₆) δ ppm 168.5, 156.5, 153.2, 150.7, 148.9, 139.8, 137.7, 137.3, 136.0, 135.6, 133.8, 130.2, 129.0, 128.3, 122.1, 119.9, 115.7, 74.3, 64.4, 61.7, 59.0, 50.1, 46.9, 46.0, 46.0, 43.4, 39.7, 33.6, 30.2, 27.1, 24.6, 21.0, 19.2, 15.5, 11.1; HRMS-ESI (m/z): [M+H]⁺ calculated for C₄₉H₆₀ClN₆O₄Si: 859.4134 found: 859.4130.

### Step B: (4-methoxyphenyl)methyl 3-[1-[[3-[2-[tert-butyl(diphenyl)silyl]oxyethoxy]-5,7-dimethyl-1-adamantyl]methyl]-5-methyl-pyrazol-4-yl]-6-(3-chloro-4-methyl-6,7-dihydro-5H-pyrido[2,3-c]pyridazin-8-yl)pyridine-2-carboxylate

To 1.23 g (1.43 mmol) of the product from Step A, 0.35 mL (2 eq) of (4-methoxyphenyl)methanol, 748 mg (2 eq) of PPh₃ in 7 mL of toluene was added 0.56 mL (2 eq) of DIAD dropwise, and the mixture was stirred at 50° C. until complete conversion. The product was purified by column chromatography (silica gel, using DCM and EtOAc as eluents) to give 1.11 g (79%) of the desired product. ¹H NMR (500 MHz, dmso-d₆) δ ppm 7.84 (d, 1H), 7.67 (d, 1H), 7.65 (d, 4H), 7.44 (t, 2H), 7.41 (s, 1H), 7.40 (t, 4H), 7.15 (d, 2H), 6.87 (d, 2H), 5.07 (s, 2H), 3.96 (t, 2H), 3.83 (s, 2H), 3.71 (s, 3H), 3.66 (t, 2H), 3.45 (t, 2H), 2.86 (t, 2H), 2.29 (s, 3H), 2.08 (s, 3H), 1.97 (qn, 2H), 1.38 (s, 2H), 1.25/1.18 (d+d, 4H), 1.18/1.12 (d+d, 4H), 1.01/0.93 (d+d, 2H), 0.97 (s, 9H), 0.82 (s, 6H); ¹³C NMR (500 MHz, dmso-d₆) δ ppm 166.8, 159.7, 156.3, 153.6, 150.8, 147.7, 140.1, 137.6, 137.3, 136.0, 135.6, 133.8, 130.2, 130.2, 129.1, 128.2, 127.7, 123.0, 120.4, 115.6, 114.3, 74.2, 66.8, 64.4, 61.7, 59.3, 55.6, 49.9, 46.8, 46.0, 46.0, 43.3, 39.7, 33.6, 30.1, 27.1, 24.6, 21.0, 19.3, 15.5, 10.8; HRMS-ESI (m/z): [M+H]⁺ calculated for C₅₇H₆₈ClN₆O₅Si: 979.4709 found: 979.4710.

### Step C: (4-methoxyphenyl)methyl 6-(3-chloro-4-methyl-6,7-dihydro-5H-pyrido[2,3-c]pyridazin-8-yl)-3-[1-[[3-(2-hydroxyethoxy)—5,7-dimethyl-1-adamantyl]methyl]-5-methyl-pyrazol-4-yl]pyridine-2-carboxylate

To 45.4 g (46.3 mmol) of the product from Step Bin 470 mL of THF was added 51 mL (1.1 eq) of a 1 M solution of TBAF in THF and mixture was stirred for 2 h. After quenching with a saturated NH₄Cl solution, the mixture was extracted with EtOAc and the organic phase was dried and purified by column chromatography (silica gel, using DCM and MeOH as eluents) to give 21.6 g (63%) of the desired product. ¹H NMR (500 MHz, dmso-d₆) δ ppm 7.85 (d, 1H), 7.70 (d, 1H), 7.39 (s, 1H), 7.18 (d, 2H), 6.90 (d, 2H), 5.10 (s, 2H), 4.45 (t, 1H), 3.96 (t, 2H), 3.84 (s, 2H), 3.74 (s, 3H), 3.40 (q, 2H), 3.33 (t, 2H), 2.86 (t, 2H), 2.29 (s, 3H), 2.09 (s, 3H), 1.98 (qn, 2H), 1.39 (s, 2H), 1.27/1.21 (d+d, 4H), 1.18/1.12 (d+d, 4H), 1.03/0.94 (d+d, 2H), 0.84 (s, 6H); ¹³C NMR (500 MHz, dmso-d₆) δ ppm 166.8, 159.7, 156.3, 153.6, 150.8, 147.8, 140.2, 137.6, 137.3, 136, 130.2, 129.1, 127.7, 123.0, 120.4, 115.6, 114.3, 74.0, 66.8, 62.2, 61.5, 59.0, 55.6, 50.0, 46.9, 46.0, 46.0, 43.3, 39.7, 33.5, 30.1, 24.6, 21.0, 15.5, 10.9; HRMS-ESI (m/z): [M+H]⁺ calculated for C₄₁H₅₀ClN₆O₅: 741.3531 found: 741.3530.

### Step D: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-(2-hydroxyethoxy)—5,7-dimethyl-1-adamantyl]methyl]-5-methyl-pyrazol-4-yl]pyridine-2-carboxylate

The mixture of 7.1 g (9.6 mmol) of the product from Step C, 2.8 g (19 mmol) of 1,3-benzothiazol-2-amine, 4.8 mL (28 mmol) of N-ethyl-N-isopropyl-propan-2-amine, 861 mg (0.94 mmol) of Pd₂(dba)₃ and 1.1 g (1.9 mmol) of XantPhos in 66 mL of cyclohexanol was stirred at 130° C. for 2 h. The product was purified by column chromatography (silica gel, using DCM and MeOH as eluents) to give 5.71 g (63%) of desired product. ¹H NMR (500 MHz, dmso-d₆) δ ppm 7.95 (d, 1H), 7.81 (brd, 1H), 7.69 (d, 1H), 7.49 (brs, 1H), 7.39 (s, 1H), 7.35 (m, 1H), 7.19 (m, 2H), 7.16 (m, 1H), 6.91 (m, 2H), 5.10 (s, 2H), 4.46 (t, 1H), 3.99 (m, 2H), 3.85 (s, 2H), 3.75 (s, 3H), 3.40 (m, 2H), 3.34 (t, 2H), 2.85 (t, 2H), 2.32 (s, 3H), 2.11 (s, 3H), 1.99 (m, 2H), 1.45-0.9 (m, 12H), 0.84 (s, 6H); HRMS-ESI (m/z): [M+H]⁺ calculated for C₄₈H₅₅N₈O₅S: 855.4016 found: 855.4011.

### Step E: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-[2-(p-tolylsulfonyloxy)ethoxy]-1-adamantyl]methyl]-5-methyl-pyrazol-4-yl]pyridine-2-carboxylate

To 5.0 g (5.8 mmol) of the product from Step Din 50 mL of dichloromethane were added 2.5 mL (3.1 eq.) of N,N-diethylethanamine and 2.9 g (1.5 eq) of p-tolylsulfonyl 4-methylbenzenesulfonate, then the mixture was stirred for 18 h. The product was purified by column chromatography (silica gel, using DCM and EtOAc as eluents) to give 2.95 g (50%) of the desired product. ¹H NMR (500 MHz, dmso-d₆) δ ppm 7.95 (d, 1H), 7.81 (brs, 1H), 7.76 (m, 2H), 7.45 (brs, 1H), 7.45 (m, 2H), 7.40 (s, 1H), 7.35 (m, 1H), 7.18 (m, 2H), 7.17 (m, 1H), 6.97 (d, 1H), 6.90 (m, 2H), 5.10 (s, 2H), 4.05 (m, 2H), 4.00 (m, 2H), 3.82 (s, 2H), 3.74 (s, 3H), 3.47 (m, 2H), 2.85 (m, 2H), 2.40 (s, 3H), 2.32 (s, 3H), 2.10 (s, 3H), 1.98 (m, 2H), 1.87-1.34 (m, 12H), 0.81 (s, 6H); HRMS-ESI (m/z): [M+Na]⁺ calculated for C₅₅H₆₁N₈O₇S₂: 1009.4105 found: 1009.4102.

### Preparation 17: tert-butyl-[2-[[3-[[5-methyl-4-(4,4,5,5-tetramethyl-1,3,2-dioxaborolan-2-yl)pyrazol-1-yl]methyl]-1-adamantyl]oxy]ethoxy]-diphenyl-silane

### Step A: (3-bromo-1-adamantyl)methanol

To 3-bromoadamantane-1-carboxylic acid (10.0 g, 38.6 mmol) in THF (25 mL) was added slowly a 1 M solution of BH3-THF in THF (115 mL, 3 eq), and the mixture was stirred for 48 h. After the addition of methanol and stirring for 30 min, purification by column chromatography (silica gel, heptane and MTBE as eluents) afforded the desired product (8.37 g, 88%). ¹H NMR (400 MHz, DMSO-d₆): δ ppm 4.50 (t, 1H), 3.02 (d, 2H), 2.28/2.21 (dm+dm, 4H), 2.11 (m, 2H), 2.07 (s, 2H), 1.66/1.56 (dm+dm, 2H), 1.48/1.39 (dm+dm, 4H); ¹³C NMR (100 MHz, DMSO-d₆) δ ppm 70.9, 69.3, 51.3, 49.0, 40.6, 37.3, 35.1, 32.3.

### Step B: 1-[(3-bromo-1-adamantyl)methyl]pyrazole

To the product from Step A (8.37 g, 34.1 mmol), 1H-pyrazole (2.79 g, 1.2 eq) in toluene (100 mL) was added (cyanomethylene)tributylphosphorane (10.7 mL, 1.2 eq) and the reaction mixture was stirred at 90° C. for 2 h. Purification by column chromatography (silica gel, heptane and MTBE as eluents) afforded the desired product (8.50 g, 84%). ¹H NMR (400 MHz, DMSO-d₆): δ ppm 7.63 (dd, 1H), 7.43 (dd, 1H), 6.23 (t, 1H), 3.87 (s, 2H), 2.24/2.13 (m+m, 4H), 2.10 (m, 2H), 2.07 (s, 2H), 1.63/1.50 (m+m, 2H), 1.47/1.43 (m+m, 4H); ¹³C NMR (100 MHz, DMSO-d₆) δ ppm 138.9, 131.7, 105.1, 68.0, 61.8, 51.8, 48.5, 39.8, 38.3, 34.6, 32.1; HRMS-ESI (m/z): [M+H]⁺ calculated for C₁₄H₂₀BrN₂: 295.0810 found: 295.0804.

### Step C: 1-[(3-bromo-1-adamantyl)methyl]-5-methyl-pyrazole

To the product from Step B (1.70 g, 5.76 mmol) in THF (30 mL) was added butyllithium (2.5 M in THF, 12 mL, 5 eq) at −78° C. After 1 h, iodomethane (7.2 mL, 5 eq) was added to the mixture. After 10 min, the reaction mixture was quenched with a saturated solution of NH₄Cl, extracted with EtOAc and the combined organic layers were dried and concentrated to give the desired product (2.0 g, 112%), which was used in the next step without further purification. ¹H NMR (400 MHz, DMSO-d₆): δ ppm 7.31 (d, 1H), 6.01 (d, 1H), 3.76 (s, 2H), 2.25/2.15 (d+d, 4H), 2.24 (s, 3H), 2.16 (s, 2H), 2.10 (m, 2H), 1.63/1.52 (d+d, 2H), 1.52/1.49 (d+d, 4H); ¹³C NMR (100 MHz, DMSO-d₆) δ ppm 139.2, 138.0, 105.2, 68.2, 58.3, 52.1, 48.5, 40.5, 38.4, 34.5, 32.2, 11.8; HRMS-ESI (m/z): [M+H]⁺ calculated for C₁₅H₂₂BrN₂: 309.0966 found: 309.0962.

### Step D: 2-[[3-[(5-methylpyrazol-1-yl)methyl]-1-adamantyl]oxy]ethanol

The mixture of the product from Step C (2.00 g, 6.47 mmol), ethylene glycol (14.4 mL, 40 eq), and DIPEA (5.6 mL, 5 eq) was stirred at 120° C. for 6 h. After diluting with water and extracting with EtOAc, the combined organic phases were purified by column chromatography (silica gel, heptane and MTBE as eluents) to give the desired product (1.62 g, 86.6%). ¹H NMR (400 MHz, DMSO-d₆): δ ppm 7.28 (d, 1H), 5.99 (m, 1H), 4.46 (t, 1H), 3.75 (s, 2H), 3.40 (m, 2H), 3.32 (m, 2H), 2.23 (brs, 3H), 2.13 (m, 2H), 1.61/1.52 (m+m, 4H), 1.47/1.43 (m+m, 2H), 1.45 (s, 2H), 1.44-1.35 (m, 4H); ¹³C NMR (100 MHz, DMSO-d₆) δ ppm 137.8, 105.1, 61.8, 61.5, 59.0, 44.6, 40.8, 39.6, 35.7, 30.0, 11.9; HRMS-ESI (m/z): [M+H]⁺ calculated for C₁₇H₂₇N₂O₂: 291.2073 found: 291.2069.

### Step E: tert-butyl-[2-[[3-[(5-methylpyrazol-1-yl)methyl]-1-adamantyl]oxy]ethoxy]-diphenyl-silane

To the product from Step D (6.52 g, 22.5 mmol) and imidazole (2.29 g, 1.5 eq) in DCM (67 ml) was added tert-butyl-chloro-diphenyl-silane (6.9 mL, 1.2 eq) and the reaction mixture was stirred for 1 h. Purification by column chromatography (silica gel, heptane and MTBE as eluents) afforded the desired product (11.0 g, 92.7%). LC/MS (C₃₃H₄₅N₂O₂Si) 529 [M+H]⁺.

### Step F: tert-butyl-[2-[[3-[(4-iodo-5-methyl-pyrazol-1-yl)methyl]-1-adamantyl]oxy]ethoxy]-diphenyl-silane

To the product from Step E (11.0 g, 20.8 mmol) in DMF (105 mL) was added N-iodosuccinimide (5.85 g, 1.25 eq.) and the reaction mixture was stirred for 3 h. After the reaction mixture was diluted with water and extracted with DCM, the combined organic phases were washed with saturated sodium thiosulphate and brine, dried, and evaporated to get the desired product (11.0 g, 81%). ¹H NMR (400 MHz, DMSO-d₆): δ ppm 7.70-7.36 (m, 10H), 7.44 (s, 1H), 3.86 (s, 2H), 3.67 (t, 2H), 3.45 (t, 2H), 2.24 (s, 3H), 2.12 (m, 2H), 1.66-1.32 (m, 12H), 0.98 (s, 9H) ¹³C NMR (100 MHz, DMSO-d₆) δ ppm 142.4, 140.9, 64.4, 61.4, 60.4, 60.3, 30.0, 27.1, 12.2; HRMS-ESI (m/z): [M+H]⁺ calculated for C₃₃H₄₄IN₂O₂Si: 655.2217 found: 655.2217.

### Step G: tert-butyl-[2-[[3-[[5-methyl-4-(4,4,5,5-tetramethyl-1,3,2-dioxaborolan-2-yl)pyrazol-1-yl]methyl]-1-adamantyl]oxy]ethoxy]-diphenyl-silane

To the product from Step F (11.0 g, 16.8 mmol) in THF (84 mL) was added chloro(isopropyl)magnesium-LiCl (1.3 M in THF, 17 mL, 1.2 eq) at 0° C., and the reaction mixture was stirred for 40 min, treated with 2-isopropoxy-4,4,5,5-tetramethyl-1,3,2-dioxaborolane (10.3 mL, 3 eq), and stirred for 10 min. After dilution with a saturated solution NH₄Cl and extraction with EtOAc, the combined organic phases were concentrated and purified by column chromatography (silica gel, heptane and MTBE as eluents) to give the desired product (9.0 g, 82%). ¹H NMR (400 MHz, DMSO-d₆): δ ppm 7.66 (d, 4H), 7.47 (s, 1H), 7.45 (t, 2H), 7.40 (t, 4H), 3.77 (s, 2H), 3.67 (t, 2H), 3.44 (t, 2H), 2.36 (s, 3H), 2.11 (br, 2H), 1.60/1.48 (d+d, 4H), 1.44 (d, 2H), 1.44 (s, 2H), 1.40 (d, 4H), 1.23 (s, 12H), 0.97 (s, 9H); ¹³C NMR (100 MHz, DMSO-d₆) δ ppm 146.9, 144.2, 133.8, 130.2, 128.3, 125.7, 104.6, 83.0, 72.5, 64.4, 61.4, 58.9, 44.6, 40.7, 39.6, 38.7, 35.6, 30.0, 27.1, 25.2, 19.3, 12.1; HRMS-ESI (m/z): [M+H]⁺ calculated for C₃₉H₅₆BN₂O₂Si: 655.4102 found: 655.4108.

### Preparation 18: (4-methoxyphenyl)methyl 6-[3-(1,3-benzothiazol-2-ylamino)-4-methyl-6,7-dihydro-5H-pyrido[2,3-c]pyridazin-8-yl]-3-[5-methyl-1-[[3-[2-(p-tolylsulfonyloxy)ethoxy]-1-adamantyl]methyl]pyrazol-4-yl]pyridine-2-carboxylate

### Step A: (4-methoxyphenyl)methyl 3-[1-[[3-[2-[tert-butyl(diphenyl)silyl]oxyethoxy]-1-adamantyl]methyl]-5-methyl-pyrazol-4-yl]-6-[3-(3,6-dichloro-5-methyl-pyridazin-4-yl)propylamino]pyridine-2-carboxylate

The mixture of the product from Preparation 11 (3.67 g, 6.79 mmol), the product from Preparation 17 (4.89 g, 1.1 eq), Pd(AtaPhos)₂Cl₂ (301 mg, 0.1 eq), and Cs₂CO₃ (6.64 g, 3 eq) in 1,4-dioxane (41 mL) and H₂O (6.8 mL) was stirred at 80° C. for 12 h. Purification by column chromatography (silica gel, heptane and EtOAc as eluents) afforded the desired product (3.0 g, 45%). ¹H NMR (400 MHz, DMSO-d₆): δ ppm 7.69-7.37 (m, 10H), 7.31 (d, 1H), 7.24 (s, 1H), 7.12 (m, 2H), 6.98 (t, 1H), 6.83 (m, 2H), 6.62 (d, 1H), 4.99 (s, 2H), 3.76 (s, 2H), 3.70 (s, 3H), 3.66 (t, 2H), 3.45 (t, 2H), 3.35 (m, 2H), 2.85 (m, 2H), 2.34 (s, 3H), 2.12 (m, 2H), 2.02 (s, 3H), 1.77 (m, 2H), 1.65-1.33 (m, 12H), 0.97 (s, 9H); HRMS-ESI (m/z): [M+H]⁺ calculated for C₅₅H₆₅Cl₂N₆O₅Si: 987.4163 found: 987.4158.

### Step B: (4-methoxyphenyl)methyl 3-[1-[[3-[2-[tert-butyl(diphenyl)silyl]oxyethoxy]-1-adamantyl]methyl]-5-methyl-pyrazol-4-yl]-6-(3-chloro-4-methyl-6,7-dihydro-5H-pyrido[2,3-c]pyridazin-8-yl)pyridine-2-carboxylate

The mixture of the product from Step A (3.00 g, 3.00 mmol), Cs₂CO₃ (1.95 g, 2 eq), DIPEA (1.0 mL, 2 eq), and Pd(Ataphos)₂Cl₂ (212 mg, 0.1 eq) in 1,4-dioxane (15 mL) was stirred at 110° C. for 18 h. Purification by column chromatography (silica gel, DCM and MeOH as eluents) afforded the desired product (1.74 g, 60%). ¹H NMR (400 MHz, DMSO-d₆): δ ppm 7.84 (d, 1H), 7.68 (d, 1H), 7.68-7.37 (m, 10H), 7.36 (s, 1H), 7.16 (m, 2H), 6.87 (m, 2H), 5.08 (s, 2H), 3.96 (m, 2H), 3.81 (s, 2H), 3.72 (s, 3H), 3.67 (t, 2H), 3.46 (t, 2H), 2.87 (t, 2H), 2.29 (s, 3H), 2.13 (m, 2H), 2.09 (s, 3H), 1.98 (m, 2H), 1.65-1.37 (m, 12H), 0.97 (s, 9H); HRMS-ESI (m/z): [M+H]+ calculated for C₅₅H₆₄ClN₆O₅Si: 951.4396 found: 951.4397.

### Step C: (4-methoxyphenyl)methyl 6-(3-chloro-4-methyl-6,7-dihydro-5H-pyrido[2,3-c]pyridazin-8-yl)-3-[1-[[3-(2-hydroxyethoxy)-1-adamantyl]methyl]-5-methyl-pyrazol-4-yl]pyridine-2-carboxylate

To the product from Step B (1.73 g, 1.82 mmol) in THF (20 mL) was added a 1 M solution of TBAF in THF (2.0 mL, 1.1 eq) at 0° C. and the reaction mixture was stirred for 2 h. Purification by column chromatography (silica gel, DCM and MeOH as eluents) afforded the desired product (1.06 g, 82%). ¹H NMR (400 MHz, DMSO-d₆): δ ppm 7.85 (d, 1H), 7.71 (d, 1H), 7.36 (s, 1H), 7.19 (m, 2H), 6.90 (m, 2H), 5.10 (s, 2H), 4.47 (t, 1H), 3.96 (m, 2H), 3.81 (s, 2H), 3.75 (s, 3H), 3.40 (m, 2H), 3.34 (t, 2H), 2.87 (t, 2H), 2.29 (s, 3H), 2.14 (m, 2H), 2.10 (s, 3H), 1.98 (m, 2H), 1.67-1.36 (m, 12H); HRMS-ESI (m/z): [M+H]⁺ calculated for C₃₉H₄₆ClN₆O₅: 713.3218 found: 713.3217.

### Step D: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-(2-hydroxyethoxy)-1-adamantyl]methyl]-5-methyl-pyrazol-4-yl]pyridine-2-carboxylate

The mixture of the product from Step C (1.00 g, 1.40 mmol), 1,3-benzothiazol-2-amine (421 mg, 2 eq), Pd₂(dba)₃ (128 mg, 0.1 eq), XantPhos (162 mg, 0.2 eq), and DIPEA (0.72 mL, 3 eq) in cyclohexanol (10 mL) was stirred at 130° C. for 1 h. Purification by column chromatography (silica gel, heptane, then DCM and MeOH as eluents) afforded the desired product (600 mg, 53%). ¹H NMR (400 MHz, DMSO-d₆): δ ppm 12.18/10.84 (brs/brs, 1H), 7.94 (d, 1H), 7.83 (br, 1H), 7.69 (d, 1H), 7.57 (br, 1H), 7.36 (s, 1H), 7.35 (brt, 1H), 7.20 (d, 2H), 7.17 (brt, 1H), 6.91 (d, 2H), 5.11 (s, 2H), 4.47 (brt, 1H), 4.00 (t, 2H), 3.81 (s, 2H), 3.75 (s, 3H), 3.41 (brq, 2H), 3.35 (t, 2H), 2.85 (t, 2H), 2.32 (s, 3H), 2.14 (m, 2H), 2.12 (s, 3H), 1.99 (qn, 2H), 1.62/1.53 (d+d, 4H), 1.53 (s, 2H), 1.49/1.44 (d+d, 2H), 1.44 (s, 4H); ¹³C NMR (100 MHz, DMSO-d₆) δ ppm 139.9, 137.6, 130.1, 126.4, 122.4, 122.0, 118.9, 114.2, 66.7, 61.9, 61.5, 59.5, 55.6, 45.4, 44.7, 40.8, 39.5, 35.6, 30.1, 24.3, 21.7, 12.6, 10.8; HRMS-ESI (m/z): [M+H]⁺ calculated for C₄₆H₅₁N₈O₅S: 827.3703 found: 827.3709.

### Step E: (4-methoxyphenyl)methyl 6-[3-(1,3-benzothiazol-2-ylamino)-4-methyl-6,7-dihydro-5H-pyrido[2,3-c]pyridazin-8-yl]-3-[5-methyl-1-[[3-[2-(p-tolylsulfonyloxy)ethoxy]-1-adamantyl]methyl]pyrazol-4-yl]pyridine-2-carboxylate

To the product from Step D (600 mg, 0.726 mmol) and N,N-diethylethanamine (0.31 mL, 3 eq) in dichloromethane (7 mL) was added p-tolylsulfonyl 4-methylbenzenesulfonate (357 mg, 1.5 eq) and the reaction mixture was stirred for 18 h. Purification by flash chromatography (silica gel, using DCM and MeOH as eluents) afforded 354 mg (50%) of the desired product. ¹H NMR (500 MHz, dmso-d₆) δ ppm 12.22/10.85 (brs/brs, 1H), 7.94 (d, 1H), 7.81 (br, 1H), 7.77 (d, 2H), 7.70 (d, 1H), 7.52 (br, 1H), 7.45 (d, 2H), 7.37 (s, 1H), 7.35 (t, 1H), 7.19 (d, 2H), 7.17 (t, 1H), 6.89 (d, 2H), 5.10 (s, 2H), 4.05 (t, 2H), 4.00 (t, 2H), 3.79 (s, 2H), 3.74 (s, 3H), 3.49 (t, 2H), 2.86 (t, 2H), 2.40 (s, 3H), 2.32 (s, 3H), 2.11 (m, 2H), 2.11 (s, 3H), 1.99 (qn, 2H), 1.55-1.36 (m, 12H); ¹³C NMR (500 MHz, dmso-d₆) δ ppm 139.9, 137.6, 130.5, 130.3, 128.1, 126.4, 122.4, 122.0, 118.9, 114.2, 71.4, 66.8, 59.4, 58.2, 55.6, 45.4, 30.0, 24.2, 21.6, 21.6, 12.6, 10.9; HRMS-ESI (m/z): [M+H]⁺ calculated for C₅₃H₅₇N₈O₇S₂: 981.3792 found: 981.3795.

### Preparation 1b_01: Methyl 2-(tert-butoxycarbonylamino)-5-[3-[4-[3-[tert-butoxycarbonyl(methyl)amino]prop-1-ynyl]-2-fluoro-phenoxy]propyl]thiazole-4-carboxylate

A 500 mL oven-dried, one-necked, round-bottom flask was equipped with a PTFE-coated magnetic stirring bar and fitted with a reflux condenser. It was charged with 13.41 g of Preparation 1a (25 mmol, 1 eq.), 8.46 g of tert-butyl N-methyl-N-prop-2-ynyl-carbamate (50 mmol, 2 eq.) and 50 mL of DIPA (36.10 g, 50 mL, 356.8 mmol, 14.27 eq.) then 125 ml of dry THF was added and the system was flushed with argon. After 5 minutes stirring under inert atmosphere 549 mg of Pd(PPh₃)₂Cl₂ (1.25 mmol, 0.05 eq.) and 238 mg of CuI (1.25 mmol, 0.05 eq.) were added. The resulting mixture was then warmed up to 60° C. and stirred at that temperature until no further conversion was observed. Celite was added to the reaction mixture and the volatiles were removed under reduced pressure. Then it was purified via flash column chromatography using heptane and EtOAc as eluents to give 10.5 g (18.2 mmol, 73%) of the desired product. ¹H NMR (500 MHz, DMSO-d₆) δ ppm 11.65 (br s, 1H), 7.31 (br d, 1H), 7.21 (br d, 1H), 7.14 (t, 1H), 4.23 (s, 2H), 4.10 (t, 2H), 3.73 (s, 3H), 3.23 (t, 2H), 2.86 (s, 3H), 2.07 (m, 2H), 1.46/1.41 (s, 18H); ¹³C NMR (125 MHz, DMSO-d₆) δ ppm 129.1, 119.2, 115.4, 68.1, 51.9, 38.6, 33.8, 30.5, 23.2; HRMS-ESI (m/z): [M+H]⁺ calculated for C₂₈H₃₇FN₃O₇S: 578.2331, found 578.2331.

### Preparation 2a_01: 5-[tert-Butyl(dimethyl)silyl]oxy-4-[tert-butyl(diphenyl)silyl]oxy-pentan-1-ol

### Step A: pent-4-enyl benzoate

30.00 g of pent-4-en-1-ol (0.35 mol, 1 eq.) and 58.5 mL of N,N-diethylethanamine (0.42 mol, 1.2 eq.) were mixed in 200 mL of DCM then cooled to 0° C. 48.5 mL of benzoyl chloride (0.42 mol, 1.2 eq.) was added to the mixture at 0° C. via dropping funnel under inert atmosphere. After the addition the mixture was further stirred at 0° C. for 30 min then at rt for on. The mixture was diluted with 100 mL of DCM then the organic phase was washed with water, 1 M NaOH, 1 M HCl, brine, respectively. The organic phase was dried over MgSO₄, filtered, concentrated and purified via flash column chromatography using heptane and EtOAc as eluents to give 63.19 g (95%) of the desired product as colorless liquid. ¹H NMR (500 MHz, DMSO-d₆) δ ppm 7.97 (dd, 2H), 7.66 (t, 1H), 7.53 (t, 2H), 5.91-5.81 (m, 1H), 5.09-4.97 (m, 2H), 4.27 (t, 2H), 2.17 (q, 2H), 1.81 (qv, 2H); ¹³C NMR (125 MHz, DMSO-d₆) δ ppm 166.2, 138.2, 133.8, 130.3, 129.6, 129.2, 115.8, 64.5, 30.1, 27.8; GC-MS-EI (m/z): [M]⁺ calculated for C₁₂H₁₄O₂: 190.1, found 190.

### Step B: 4,5-dihydroxypentyl benzoate

42.22 g of the product from Step A (0.26 mol, 1.0 eq.), 50.40 g of 4-methyl-4-oxido-morpholin-4-ium; hydrate (0.37 mol, 1.7 eq) were mixed in 360 mL of 2-methylpropan-2-ol and 40 ml of water then 6.57 g of tetraoxoosmium (2.5 w % in 2-methylpropan-2-ol, 0.64 mmol, 0.002 eq.) was added and the mixture was stirred at 60° C. for 24 h. Full conversion was observed. The mixture was cooled down to rt and 1 M Na₂S₂O₃ was added then stirred for further 10 min at rt. DCM was added and the organic phase was separated, washed with water, brine, respectively. The solution was dried over MgSO₄, filtered, concentrated and purified via flash column chromatography using heptane and EtOAc as eluents to give 36.9 g (63%) of the desired product as white solid. ¹H NMR (500 MHz, DMSO-d₆) δ ppm 7.99-7.50 (m, 5H), 4.50 (m, 2H), 4.28 (m, 2H), 3.45 (m, 1H), 3.30-3.24 (m+m, 2H), 1.85-1.72 (m+m, 2H), 1.59-1.33 (m+m, 2H); ¹³C NMR (125 MHz, DMSO-d₆) δ ppm 166.2, 133.8-129.1, 71.2, 66.3, 65.5, 30.3, 25.2; HRMS-ESI (m/z): [M+Na]⁺ calculated for C₁₂H₁₆NaO₄: 247.0941, found 247.0941.

### Step C: 5-[tert-butyl(dimethyl)silyl]oxy-4-hydroxy-pentyl] benzoate

24.86 g of the product from Step B (0.11 mol, 1 eq) and 15.09 g of imidazole (0.22 mol, 2 eq.) were mixed in 120 mL of N,N-dimethylformamide then cooled to −20° C. under inert atmosphere. 16.71 g of tert-butyl-chloro-dimethyl-silane (0.11 mol, 1 eq.) in 40 mL of N,N-dimethylformamide was added in slow rate over a period of 30 min, supported with 10 ml of DCM then left to warm up to rt and further stirred for on. Full conversion was observed. Quenched with cc. NH₄Cl then evaporated most of the volatiles. EtOAc and water were added to the residue, the organic phase was separated then washed with water and brine, dried over MgSO₄, filtered, concentrated and purified via flash column chromatography using heptane and EtOAc as eluents to give 33.71 g (90%) of the desired product as colorless oil. ¹H NMR (500 MHz, DMSO-d₆) δ ppm 7.95 (m, 2H), 7.66 (m, 1H), 7.52 (m, 2H), 4.58 (d, 1H), 4.29 (m, 2H), 3.51-3.35 (dd+dd, 2H), 3.48 (m, 1H), 1.86-1.74 (m+m, 2H), 1.67-1.34 (m+m, 2H), 0.83 (s, 9H), 0.01 (s, 6H); ¹³C NMR (125 MHz, DMSO-d₆) δ ppm 166.2, 133.7, 130.4, 129.5, 129.2, 70.6, 67.7, 65.3, 30.2, 26.3, 24.9, −4.9.

### Step D: [5-[tert-butyl(dimethyl)silyl]oxy-4-[tert-butyl(diphenyl)silyl]oxy-pentyl] benzoate

33.51 g of the product from Step C (0.10 mol, 1 eq), 16.85 g of imidazole (0.25 mol, 2.5 eq.) and 1.21 g of N,N-dimethylpyridin-4-amine (0.01, 0.1 eq.) were mixed in 230 mL of N,N-dimethylformamide then 38 mL of tert-butyl-chloro-diphenyl-silane (0.15 mol, 1.5 eq.) was added in slow rate, supported with 20 mL of N,N-dimethylformamide then stirred at 50° C. for overnight. Full conversion was observed. The mixture was cooled to rt, quenched with cc. NH₄Cl then evaporated most of the volatiles. EtOAc and water were added to the residue, the organic phase was separated then washed with water and brine, dried over MgSO₄, filtered, concentrated and purified via flash column chromatography using heptane and EtOAc as eluents to give 56.43 g (99%) of the desired product as colorless thick oil. ¹H NMR (500 MHz, DMSO-d₆) δ ppm 7.91-7.37 (m, 15H), 4.17 (m, 2H), 3.76 (m, 1H), 3.45 (m, 2H), 1.72 (m, 2H), 1.66-1.57 (m+m, 2H), 0.99 (s, 9H), 0.74 (s, 9H), −0.12/−0.16 (s+s, 6H); ¹³C NMR (125 MHz, DMSO-d₆) δ ppm 166.1, 136.0-128.0, 73.3, 66.0, 65.1, 30.3, 27.3, 26.1, 24.0, −5.1; HRMS-ESI (m/z): [M+Na]⁺ calculated for C₃₄H₄₈NaO₄Si₂: 599.2983, found 599.2981.

### Step E: 5-[tert-butyl(dimethyl)silyl]oxy-4-[tert-butyl(diphenyl)silyl]oxy-pentan-1-ol

46.10 g of the product from Step D (0.08 mol, 1 eq) was dissolved in 227 mL of MeOH and 117 mL of THF then 12.79 g of NaOH (0.32 mol, 4.0 eq.) in 85 ml of water was added slowly while the mixture was cooled with ice. After the addition the mixture left to stir at rt until full conversion was observed (ca. 4 h). EtOAc and water were added then separated and the organic phase was washed with brine, dried over MgSO₄, filtered, concentrated and purified via flash column chromatography using heptane and EtOAc as eluents to give 29.32 g (78%) of the desired product as colorless oil. ¹H NMR (500 MHz, DMSO-d₆) δ ppm 7.65-7.37 (m, 10H), 4.34 (t, 1H), 3.71 (m, 1H), 3.42 (m, 2H), 3.26 (m, 2H), 1.52 (m, 2H), 1.42 (m, 2H), 0.99 (s, 9H), 0.77 (s, 9H), −0.13 (s, 6H); ¹³C NMR (125 MHz, DMSO-d₆) δ ppm 135.8, 135.8, 134.3, 134.0, 130.3, 130.2, 128.2, 128.0, 74.0, 66.4, 61.4, 30.4, 28.3, 27.3, 26.2, −5.1; HRMS-ESI (m/z): [M+Na]⁺ calculated for C₂₇H₄₄NaO₃Si₂: 495.2721, found 495.2706.

### Preparation 3a_01: Methyl 5-[3-[4-[3-[tert-butoxycarbonyl(methyl)amino]prop-1-ynyl]-2-fluoro-phenoxy]propyl]-2-[[5-[tert-butyl(dimethyl)silyl]oxy-4-[tert-butyl(diphenyl)silyl]oxy-pentyl]amino]thiazole-4-carboxylate

### Step A: methyl 2-[tert-butoxycarbonyl-[5-[tert-butyl(dimethyl)silyl]oxy-4-[tert-butyl(diphenyl)silyl]oxy-pentyl]amino]-5-[3-[4-[3-[tert-butoxycarbonyl(methyl)amino]prop-1-ynyl]-2-fluoro-phenoxy]propyl]thiazole-4-carboxylate

Using Mitsunobu General Procedure II starting from Preparation 1b_01 as the appropriate carbamate and Preparation 2a_01 as the appropriate alcohol, 2.5 g (61%) of the desired product was obtained. ¹H NMR (500 MHz, DMSO-d₆) δ ppm 7.60-7.33 (m, 10H), 7.28 (dd, 1H), 7.17 (m, 1H), 7.1 (t, 1H), 4.22 (s, 2H), 4.09 (t, 2H), 3.94 (m, 2H), 3.71 (s, 3H), 3.67 (m, 1H), 3.38 (m, 2H), 3.22 (t, 2H), 2.85 (s, 3H), 2.07 (m, 2H), 1.65 (m, 2H), 1.48 (m, 2H), 1.45/1.40 (s+s, 18H), 0.93 (s, 9H), 0.71 (s, 9H), −0.17/−0.22 (s+s, 6H); ¹³C NMR (125 MHz, DMSO-d₆) δ ppm 147.4, 129, 119.3, 115.4, 85.1, 82.3, 73.3, 68.1, 65.6, 51.9, 46.5, 38.4, 33.8, 30.5, 30.5, 28.5/28, 27.2, 26.0, 23.1, 23.0, −5.3; HRMS-ESI (m/z): [M+H]⁺ calculated for C₅₅H₇₉FN₃O₉SSi₂: 1032.5054, found 1032.5060.

### Step B: methyl 5-[3-[4-[3-[tert-butoxycarbonyl(methyl)amino]prop-1-ynyl]-2-fluoro-phenoxy]propyl]-2-[[5-[tert-butyl(dimethyl)silyl]oxy-4-[tert-butyl(diphenyl)silyl]oxy-pentyl]amino]thiazole-4-carboxylate

Using Deprotection with HFIP General Procedure starting from the product from Step A as the appropriate carbamate, 1.2 g (53%) of the desired product was obtained. ¹H NMR (500 MHz, DMSO-d₆) δ ppm 7.68-7.35 (m, 10H), 7.56 (t, 1H), 7.30 (d, 1H), 7.20 (d, 1H), 7.11 (t, 1H), 4.22 (br., 2H), 4.07 (t, 2H), 3.70 (m, 1H), 3.68 (s, 3H), 3.42/3.38 (dd+dd, 2H), 3.11 (t, 2H), 3.04 (brq., 2H), 2.86 (br., 3H), 1.99 (quint., 2H), 1.54 (m, 2H), 1.53/1.45 (m+m, 2H), 1.41 (s, 9H), 0.97 (s, 9H), 0.74 (s, 9H), −0.14/−0.18 (s+s, 6H); ¹³C NMR (125 MHz, DMSO-d₆) δ ppm 164.6, 163.0, 154.9, 151.4, 147.5, 136.9, 136.0, 129.1, 119.3, 115.4, 114.8, 85.2, 82.3, 79.8, 73.6, 68.0, 66.2, 51.7, 44.7, 38.5, 33.8, 31.1, 30.6, 28.5, 27.2, 26.2, 24.3, 23.3, 19.4, 18.3, −5.2; HRMS-ESI (m/z): [M+H]⁺ calculated for C₅₀H₇₁FN₃O₇SSi₂: 932.4530, found 932.4526.

### Preparation 3e_01: Ethyl 5-(3-chloropropyl)-2-(methylamino)thiazole-4-carboxylate

A suspension of 2.25 g of methylthiourea (25.0 mmol, 1 eq.) in 100 mL of ethanol was cooled to 0° C., and then 7.46 g of ethyl 3-bromo-6-chloro-2-oxo-hexanoate (27.5 mmol, 1.1 eq.) was added dropwise at this temperature. After 15 min stirring at 0° C., 7 mL of TEA (5.06 g, 50 mmol, 2 eq.) was added. The resulting mixture was stirred overnight at rt. Full conversion was observed. The volatiles were removed in vacuo, then the resultant residue was portioned between EtOAc and water. The layers were separated then the organic layer was washed with water then followed with brine. The combined organic layers were dried over Na₂SO₄, filtered and the filtrate was concentrated under reduced pressure. Then it was purified via flash column chromatography using heptane and EtOAc as eluents to give 5 g (76%) of the desired product. ¹H NMR (400 MHz, DMSO-d₆) δ ppm 7.55 (q, 1H), 4.21 (q, 2H), 3.65 (t, 2H), 3.09 (m, 2H), 2.78 (d, 3H), 1.98 (m, 2H), 1.26 (t, 3H); ¹³C NMR (125 MHz, DMSO-d₆) δ ppm 165.6, 162.5, 137.4, 135.5, 60.5, 45.0, 34.1, 31.2, 24.4, 14.7; HRMS-ESI (m/z): [M+H]⁺ calculated for C₁₀H₁₆ClN₂O₂S: 263.0616, found 263.0615.

### Preparation 3h_01: Methyl 5-[3-[4-[3-[tert-butoxycarbonyl(methyl)amino]prop-1-ynyl]-2-fluoro-phenoxy]propyl]-2-[2-(2,2-dimethyl-1,3-dioxolan-4-yl)ethylamino]thiazole-4-carboxylate

### Step A: methyl 2-[tert-butoxycarbonyl-[2-(2,2-dimethyl-1,3-dioxolan-4-yl)ethyl]amino]-5-[3-(2-fluoro-4-iodo-phenoxy)propyl]thiazole-4-carboxylate

Using Mitsunobu General Procedure II starting from 2.68 g of Preparation 1a (5 mmol, 1 eq.) and 1.46 g of 2-(2,2-dimethyl-1,3-dioxolan-4-yl) ethanol (1.42 mL, 10 mmol, 2 eq.) as the appropriate alcohol, 2.8 g (84%) of the desired product was obtained. ¹H NMR (500 MHz, DMSO-d₆) δ ppm 7.57 (dd, 1H), 7.44 (dm, 1H), 6.96 (t, 1H), 4.12/4.02 (m+m, 2H), 4.07 (m, 1H), 4.05 (t, 2H), 4.02/3.54 (dd+dd, 2H), 3.75 (s, 3H), 3.21 (t, 2H), 2.06 (m, 2H), 1.86/1.82 (m+m, 2H), 1.51 (s, 9H), 1.29 (s, 3H), 1.22 (s, 3H); ¹³C NMR (125 MHz, DMSO-d₆) δ ppm 134.0, 124.9, 117.6, 73.8, 68.9, 68.1, 52.0, 44.0, 32.2, 30.5, 28.1, 27.3, 25.9, 23.1; HRMS-ESI (m/z): [M+H]⁺ calculated for C₂₆H₃₅FIN₂O₇S: 665.1188, found 665.1175.

### Step B: methyl 2-[2-(2,2-dimethyl-1,3-dioxolan-4-yl)ethylamino]-5-[3-(2-fluoro-4-iodo-phenoxy)propyl]thiazole-4-carboxylate

Using Deprotection with HFIP General Procedure starting from 2.5 g of the product from Step A (3.80 mmol) as the appropriate carbamate, 1.6 g (75%) of the desired product was obtained. ¹H NMR (500 MHz, DMSO-d₆) δ ppm 7.6 (t, 1H), 7.59 (dd, 1H), 7.45 (dm, 1H), 6.97 (dd, 1H), 4.10 (m, 1H), 4.03 (t, 2H), 4.01/3.48 (dd+dd, 2H), 3.69 (s, 3H), 3.27/3.19 (m+m, 2H), 3.11 (t, 2H), 1.99 (m, 2H), 1.76/1.72 (m+m, 2H), 1.31 (s, 3H), 1.25 (s, 3H); HRMS-ESI (m/z): [M+H]⁺ calculated for C₂₁H₂₇FIN₂O₅S: 565.0663, found 565.0642.

### Step C: methyl 5-[3-[4-[3-[tert-butoxycarbonyl(methyl)amino]prop-1-ynyl]-2-fluoro-phenoxy]propyl]-2-[2-(2,2-dimethyl-1,3-dioxolan-4-yl)ethylamino]thiazole-4-carboxylate

Using Sonogashira General Procedure starting from 400 mg of the product from Step B (0.71 mmol, 1 eq.) and 240 mg of tert-butyl N-methyl-N-prop-2-ynyl-carbamate (1.42 mmol, 2 eq.) as the appropriate acetylene, 300 mg (70%) of the desired product was obtained. ¹H NMR (500 MHz, DMSO-d₆) δ ppm 7.60 (t, 1H), 7.31 (brd, 1H), 7.21 (dd, 1H), 7.13 (t, 1H), 4.23 (brs, 2H), 4.09 (m, 1H), 4.07 (t, 2H), 4.00/3.48 (dd+dd, 2H), 3.69 (s, 3H), 3.27/3.19 (m+m, 2H), 3.12 (t, 2H), 2.86 (brs, 3H), 2.00 (m, 2H), 1.74 (m, 2H), 1.41 (s, 9H), 1.31 (s, 3H), 1.25 (s, 3H); ¹³C NMR (125 MHz, DMSO-d₆) δ ppm 164.5, 136.9, 136.4, 129.1, 119.3, 115.4, 85.2, 82.3, 73.8, 69.0, 68.0, 51.7, 41.4, 38.4, 33.8, 33.2, 30.6, 28.5, 27.3, 26.1, 23.3; HRMS-ESI (m/z): [M+H]⁺ calculated for C₃₀H₄₁FN₃O₇S: 606.2644, found 606.2650.

### Preparation 3n_01: Methyl 5-[3-[4-[3-[tert-butoxycarbonyl(methyl)amino]prop-1-ynyl]-2-fluoro-phenoxy]propyl]-2-[3-[tert-butyl(dimethyl)silyl]oxypropylamino]thiazole-4-carboxylate

### Step A: methyl 2-[tert-butoxycarbonyl-[3-[tert-butyl(dimethyl)silyl]oxypropyl]amino]-5-[3-[4-[3-[tert-butoxycarbonyl(methyl)amino]prop-1-ynyl]-2-fluoro-phenoxy]propyl]thiazole-4-carboxylate

Using Mitsunobu General Procedure II starting from 577 mg of Preparation 1b_01 (1 mmol, 1 eq.) as the appropriate carbamate and 380 mg of 3-[tert-butyl(dimethyl)silyl]oxypropan-1-ol (2 mmol, 2 eq.) as the appropriate alcohol, 600 mg (80%) of the desired product was obtained.

### Step B: methyl 5-[3-[4-[3-[tert-butoxycarbonyl(methyl)amino]prop-1-ynyl]-2-fluoro-phenoxy]propyl]-2-[3-[tert-butyl(dimethyl)silyl]oxypropylamino]thiazole-4-carboxylate

Using Deprotection with HFIP General Procedure starting from the product from Step A as the appropriate carbamate, 310 mg (47%) of the desired product was obtained. ¹H NMR (400 MHz, DMSO-d₆) δ ppm 7.50 (t, 1H), 7.30 (d, 1H), 7.20 (d, 1H), 7.11 (t, 1H), 4.21 (bs, 2H), 4.05 (t, 2H), 3.62 (t, 2H), 3.67 (s, 3H), 3.19 (q, 2H), 3.10 (t, 2H), 2.84 (brs, 3H), 2.04-1.94 (m, 2H), 1.74-1.63 (m, 2H), 1.40 (s, 9H), 0.84 (s, 9H), 0.00 (s, 6H).

### Preparation 4a_01: N-(6-Chloro-4-methyl-pyridazin-3-yl)-3-(2-trimethylsilylethoxymethyl)-1,3-benzothiazol-2-imine

### Step A: N-(6-chloro-4-methyl-pyridazin-3-yl)-1,3-benzothiazol-2-amine

A 2 L oven-dried, one-necked, round-bottom flask was equipped with a PTFE-coated magnetic stirring bar and fitted with a reflux condenser. It was charged with 34.0 g of 6-chloro-4-methyl-pyridazin-3-amine (237 mmol, 1 eq.), 34 mL of 2-chloro-1,3-benzothiazole (44.2 g, 260 mmol, 1.1 eq.), 124 mL of DIPEA (91.8 g, 710 mmol, 3 eq.) and 137 g of Cs₂CO₃ (710 mmol, 3 eq.), then 1 L of DMF were added and the system was flushed with argon. After 5 minutes stirring under inert atmosphere 2.01 g of Pd₂(dba)₃ (5.9 mmol, 0.025 eq.) and 6.85 g of XantPhos (11.8 mmol, 0.05 eq.) were added. The resulting mixture was then warmed up to 75° C. and stirred at that temperature for 4 hours to reach complete conversion. Reaction mixture was left to cool down to rt, then poured into 3 L of water while it was intensively stirred. After 30 min the precipitated product was removed by filtration, and then it was washed with water for 2 times (2×2 L). The product was dried overnight on high vacuum. The dried crude product was stirred in 1 L of heptane: Et₂O (3:2) for 30 min then filtered off to give 64.5 g (98%) of the desired product as green powder. ¹H NMR (500 MHz, DMSO-d₆) δ ppm 11.96 (brs, 1H), 7.86 (d, 1H), 7.65 (s, 1H), 7.51 (d, 1H), 7.38 (t, 1H), 7.21 (t, 1H), 2.37 (s, 3H); ¹³C NMR (125 MHz, DMSO-d₆) δ ppm 130.3, 129.5, 126.6, 122.8, 122.3, 17.2; HRMS-ESI (m/z): [M+H]⁺ calculated for C₁₂H₁₀ClN₄S: 277.0309, found 277.0305.

### Step B: N-(6-chloro-4-methyl-pyridazin-3-yl)-3-(2-trimethylsilylethoxymethyl)-1,3-benzothiazol-2-imine

A 2 L oven-dried, one-necked, round-bottomed flask equipped with a PTFE-coated magnetic stirring bar was charged with 64.5 g of the product from Step A (236 mmol, 1 eq.), 123 mL of DIPEA (9.16 g, 708 mmol, 3 eq.), 14.43 g of N,N-dimethylpyridin-4-amine (11.81 mmol, 0.05 eq.) in 1 L of dry DCM were cooled down to 0° C. under N₂. And during intensive mechanical stirring 46.00 mL of 2-(chloromethoxy)ethyl-trimethyl-silane (43.32 g, 259 mmol, 1.1 eq.) was added to the mixture dropwise over 5 min period of time. It was stirred at 0° C. for 30 min when the reaction reached complete conversion. 24.5 mL of water was added to the reaction mixture then Celite was added to the reaction mixture and the volatiles were removed under reduced pressure. It was purified via flash column chromatography using heptane and EtOAc as eluents to obtain 46.62 g (48%) of the desired product. ¹H NMR (500 MHz, DMSO-d₆) δ ppm 7.85 (dm, 1H), 7.72 (q, 1H), 7.53 (dm, 1H), 7.47 (m, 1H), 7.29 (m, 1H), 5.89 (s, 2H), 3.70 (m, 2H), 2.39 (d, 3H), 0.90 (m, 2H), −0.12 (s, 9H); ¹³C NMR (125 MHz, DMSO-d6) δ ppm 159.5, 158.5, 150.0, 138.1, 137.4, 129.5, 127.4, 125.5, 123.8, 123.2, 112.4, 73.0, 66.8, 17.7, 17.1, −1.0; HRMS-ESI (m/z): [M+H]⁺ calculated for C₁₈H₂₄ClN₄OSSi: 407.1123, found 407.1120.

### Preparation 5a_01: Methyl 5-[3-[4-[3-[tert-butoxycarbonyl(methyl)amino]prop-1-ynyl]-2-fluoro-phenoxy]propyl]-2-[[4-[tert-butyl(diphenyl)silyl]oxy-5-(p-tolylsulfonyloxy)pentyl]-[5-methyl-6-[(Z)-[3-(2-trimethylsilylethoxymethyl)-1,3-benzothiazol-2-ylidene]amino]pyridazin-3-yl]amino]thiazole-4-carboxylate

### Step A: methyl 5-[3-[4-[3-[tert-butoxycarbonyl(methyl)amino]prop-1-ynyl]-2-fluoro-phenoxy]propyl]-2-[[5-[tert-butyl(dimethyl)silyl]oxy-4-[tert-butyl(diphenylsilyl]oxy-pentyl]-[5-methyl-6-[(Z)-[3-(2-trimethylsilylethoxymethyl)-1,3-benzothiazol-2-ylidene]amino]pyridazin-3-yl]amino]thiazole-4-carboxylate

Using Buchwald General Procedure III starting from 12 g of Preparation 3a_01 (13 mmol) and 6.30 g of Preparation 4a_01 (15.6 mmol) as the appropriate halide, 14 g (83%) of the desired product was obtained. ¹H NMR (500 MHz, DMSO-d₆) δ ppm 7.85-7.23 (m, 14H), 7.58 (s, 1H), 7.31 (t, 1H), 7.19 (m, 1H), 7.14 (t, 1H), 5.86 (s, 2H), 4.37 (t, 2H), 4.20 (s, 2H), 4.15 (t, 2H), 3.73 (s, 3H), 3.71 (t, 2H), 3.67 (m, 1H), 3.39 (m, 2H), 3.27 (t, 2H), 2.83 (s, 3H), 2.41 (s, 3H), 2.12 (m, 2H), 1.72 (m, 2H), 1.52 (m, 2H), 1.40 (s, 9H), 0.90 (t, 2H), 0.89 (s, 9H), 0.69 (s, 9H), −0.14 (s, 9H), −0.19/−0.23 (s+s, 6H); ¹³C NMR (125 MHz, DMSO-d₆) δ ppm 147.5, 129.1, 119.3, 117.5, 115.4, 73.4, 72.3, 68.4, 66.8, 65.8, 51.8, 46.6, 38.5, 33.8, 31.0, 30.5, 28.5, 27.1, 26.1, 23.0, 22.6, 17.9, 17.8, −1.0, −5.3; HRMS-ESI (m/z): [M+H]⁺ calculated for C₆₈H₉₃FN₇O₈S₂Si₃: 1302.5813, found 1302.5819.

### Step B: methyl 5-[3-[4-[3-[tert-butoxycarbonyl(methyl)amino]prop-1-ynyl]-2-fluoro-phenoxy]propyl]-2-[[4-[tert-butyl(diphenyl)silyl]oxy-5-hydroxy-pentyl]-[5-methyl-6-[(Z)-[3-(2-trimethylsilylethoxymethyl)-1,3-benzothiazol-2-ylidene]amino]pyridazin-3-yl]amino]thiazole-4-carboxylate

A 100 mL oven-dried, one-necked, round-bottom flask was equipped with a PTFE-coated magnetic stirring bar and fitted with a reflux condenser. It was charged with 1.40 g of the product from Step A (1.1 mmol, 1 eq.) and 12 mg of camphor sulfonic acid (0.054 mmol, 0.05 eq.), 5 mL of DCM and 1 mL of MeOH. The resulting mixture was stirred overnight at rt to reach complete conversion. Reaction mixture was concentrated directly to Celite then purified by flash column chromatography using heptane and EtOAc as eluents to give 700 mg (55%) of the desired product as yellow solid. ¹H NMR (500 MHz, DMSO-d₆) δ ppm 7.85-7.14 (m, 14H), 7.56 (s, 1H), 7.32 (dd, 1H), 7.20 (m, 1H), 7.15 (t, 1H), 5.86 (s, 2H), 4.56 (t, 1H), 4.33 (m, 2H), 4.20 (s, 2H), 4.15 (t, 2H), 3.74 (s, 3H), 3.72 (t, 2H), 3.65 (m, 1H), 3.27 (t, 2H), 3.27 (t, 2H), 2.83 (s, 3H), 2.41 (s, 3H), 2.13 (m, 2H), 1.73/1.64 (m+m, 2H), 1.52 (m, 2H), 1.40 (s, 9H), 0.90 (t, 2H), 0.86 (s, 9H), −0.13 (s, 9H); ¹³C NMR (125 MHz, DMSO-d₆) δ ppm 154.9, 147.6, 129.1, 119.4, 117.5, 115.4, 82.4, 73.7, 72.9, 68.4, 66.8, 64.5, 51.9, 46.8, 38.5, 33.8, 31.0, 30.6, 28.5, 27.2, 23.1, 22.5, 17.9, 17.8, −1.0; HRMS-ESI (m/z): [M+H]⁺ calculated for C₆₂H₇₉FN₇O₈S₂Si₂: 1188.4949, found 1188.4938.

### Step C: methyl 5-[3-[4-[3-[tert-butoxycarbonyl(methyl)amino]prop-1-ynyl]-2-fluoro-phenoxy]propyl]-2-[[4-[tert-butyl(diphenyl)silyl]oxy-5-(p-tolylsulfonyloxy)pentyl]-[5-methyl-6-[(Z)-[3-(2-trimethylsilylethoxymethyl)-1,3-benzothiazol-2-ylidene]amino]pyridazin-3-yl]amino]thiazole-4-carboxylate

A 100 mL oven-dried, one-necked, round-bottom flask was equipped with a PTFE-coated magnetic stirring bar was charged with 700 mg of the product from Step B (0.58 mmol, 1 eq.) and 907 mg of N,N-dimethyl-1-(p-tolylsulfonyl)pyridin-1-ium-4-amine chloride (2.9 mmol, 5 eq.; see, e.g., Tetrahedron Lett. 2016, 57, 4620) were dissolved in 35 mL of DCM and stirred overnight at rt. Reaction reached complete conversion. Reaction mixture directly was concentrated onto Celite, and then purified by flash column chromatography using heptane and EtOAc as eluents to give 450 mg (56%) of the desired product. ¹H NMR (500 MHz, DMSO-d₆) δ ppm 7.88-7.23 (m, 14H), 7.58 (m, 2H), 7.53 (s, 1H), 7.31 (m, 2H), 7.31 (dd, 1H), 7.19 (m, 1H), 7.15 (t, 1H), 5.86 (s, 2H), 4.20 (s, 2H), 4.16 (t, 2H), 4.15 (t, 2H), 3.92 (m, 2H), 3.84 (m, 1H), 3.72 (t, 2H), 3.70 (s, 3H), 3.27 (t, 2H), 2.83 (s, 3H), 2.41 (s, 3H), 2.33 (s, 3H), 2.13 (m, 2H), 1.47 (m, 2H), 1.47 (m, 2H), 1.40 (s, 9H), 0.91 (t, 2H), 0.86 (s, 9H), −0.13 (s, 9H); ¹³C NMR (125 MHz, DMSO-d₆) δ ppm 147.5, 145.3, 130.4, 129.1, 128.0, 119.3, 117.4, 115.5, 72.9, 72.6, 70.4, 68.4, 66.8, 51.8, 46.2, 38.6, 33.8, 31.0, 30.1, 28.5, 27.0, 23.1, 22.4, 21.5, 17.8, 17.8, −1.0; HRMS-ESI (m/z): [M+H]⁺ calculated for C₆₉H₈₅FN₇O₁₀S₃Si₂: 1342.5037, found 1342.5039.

### Preparation 5g_01: Ethyl 5-(3-iodopropyl)-2-[methyl-[5-methyl-6-[(Z)-[3-(2-trimethylsilylethoxymethyl)-1,3-benzothiazol-2-ylidene]amino]pyridazin-3-yl]amino]thiazole-4-carboxylate

### Step A: ethyl 5-(3-chloropropyl)-2-[methyl-[5-methyl-6-[(Z)-[3-(2-trimethylsilylethoxymethyl)-1,3-benzothiazol-2-ylidene]amino]pyridazin-3-yl]amino]thiazole-4-carboxylate

Using Buchwald General Procedure III starting from 3.15 g of Preparation 3e_01 (12 mmol, 1.2 eq.) and 4.07 g of Preparation 4a_01 (10 mmol, 1 eq.) as the appropriate halide, 2.6 g (41%) of the desired product was obtained. ¹H NMR (500 MHz, DMSO-d₆) δ ppm 7.84 (d, 1H), 7.65 (s, 1H), 7.45 (d, 1H), 7.43 (tm, 1H), 7.25 (tm, 1H), 5.85 (s, 2H), 4.30 (q, 2H), 3.77 (s, 3H), 3.71 (t, 2H), 3.71 (t, 2H), 3.22 (t, 2H), 2.48 (s, 3H), 2.10 (quin, 2H), 1.31 (t, 3H), 0.92 (t, 2H),−0.11 (s, 9H); ¹³C NMR (125 MHz, DMSO-d₆) δ ppm 162.6, 157.4, 156.8, 155.1, 151.7, 140.5, 137.6, 137.1, 135.3, 125.6, 123.5, 123.2, 123.1, 117.6, 111.9, 72.9, 66.7, 60.7, 45.3, 35.4, 34.4, 24.3, 18.0, 17.8, 14.7, −1.0; HRMS-ESI (m/z): [M+H]⁺ calculated for C₂₈H₃₈ClN₆O₃S₂Si: 633.1899, found 633.1891.

### Step B: ethyl 5-(3-iodopropyl)-2-[methyl-[5-methyl-6-[(Z)-[3-(2-trimethylsilylethoxymethyl)-1,3-benzothiazol-2-ylidene]amino]pyridazin-3-yl]amino]thiazole-4-carboxylate

A 100 mL one-necked, round-bottomed flask was equipped with a PTFE-coated magnetic stirring bar and fitted with a reflux condenser. It was charged with 2.6 g of the product from Step A (4.10 mmol, 1 eq.), 1.23 g of NaI (8.2 mmol, 2 eq.) and 20 mL of dry acetone. The reaction mixture was warmed up to 60° C. and stirred at that temperature for 3 days, when the reaction reached complete conversion. The reaction mixture was diluted with the addition of water then the precipitated product was collected by filtration, washed with water, and then dried on high vacuum to obtain 2.5 g (84%) of the desired product. ¹H NMR (500 MHz, DMSO-d₆) δ 7.82 (d, 1H), 7.61 (s, 1H), 7.47-7.39 (m, 1H), 7.47-7.39 (m, 1H), 7.23 (t, 1H), 5.83 (s, 2H), 4.29 (q, 2H), 3.75 (s, 3H), 3.71 (t, 2H), 3.33 (t, 2H), 3.16 (t, 2H), 2.42 (s, 3H), 2.13 (quint., 2H), 1.33 (t, 3H), 0.91 (t, 2H), −0.12 (s, 9H); ¹³C NMR (125 MHz, DMSO-d₆) δ ppm 162.6, 157.3, 156.7, 155.1, 151.6, 140.2, 137.6, 137.1, 135.2, 127.1, 125.4, 123.4, 123.2, 117.5, 111.9, 72.8, 66.7, 60.7, 35.2, 35.2, 27.6, 17.8, 17.8, 14.8, 7.8, −1.0; HRMS-ESI (m/z): [M+H]⁺ calculated for C₂₈H₃₈I N₆O₃S₂Si: 725.1255, found 725.1248.

### Preparation 5j_01: Ethyl 5-(3-{2-fluoro-4-[3-(methylamino)prop-1-yn-1-yl]phenoxy}propyl)-2-[methyl(5-methyl-6-{[(2Z)-3-{[2-(trimethylsilyl)ethoxy]methyl}-2,3-dihydro-1,3-benzothiazol-2-ylidene]amino}pyridazin-3-yl)amino]-1,3-thiazole-4-carboxylate

### Step A: ethyl 5-{3-[4-(3-{[(tert-butoxy)carbonyl](methyl)amino}prop-1-yn-1-yl)-2-fluorophenoxy]propyl}-2-[methyl(5-methyl-6-{[(2Z)-3-{[2-(trimethylsilyl)ethoxy]methyl}-2,3-dihydro-1,3-benzothiazol-2-ylidene]amino}pyridazin-3-yl)amino]-1,3-thiazole-4-carboxylate

To the product from Preparation 5g_01 (1.75 g, 2.41 mmol, 1 eq) in dimethylformamide (50 mL) was added the product from Preparation 6a_01 (877 mg, 3.14 mmol, 1.3 eq) in dimethylformamide (10 mL) and cesium carbonate (2.36 g, 7.24 mmol, 3 eq) and the mixture was heated at 80° C. for 16 h. The reaction was concentrated in vacuo then partitioned between ethyl acetate and brine, and the organic phase was dried (magnesium sulfate) and concentrated in vacuo. Purification by automated flash column chromatography (CombiFlash Rf, 40 g RediSep™ silica cartridge) eluting with a gradient of 0-50% ethyl acetate in iso-heptane afforded the desired product as a yellow oil (1.75 g, 2 mmol, 83%). LC/MS (C₄₃H₅₄FN₇O₆SiS₂) 876 [M+H]⁺; RT 1.46 (LCMS-V-B2). ¹H NMR (400 MHz, DMSO-d₆) δ 7.83 (dd, 1H), 7.65 (d, J=1.1 Hz, 1H), 7.49-7.39 (m, 2H), 7.35-7.28 (m, 1H), 7.27-7.12 (m, 3H), 5.86 (s, 2H), 4.25 (q, J=7.1 Hz, 2H), 4.19 (s, 2H), 4.14 (t, J=6.1 Hz, 2H), 3.77 (s, 3H), 3.76-3.68 (m, 2H), 3.26 (t, J=7.7 Hz, 2H), 2.84 (s, 3H), 2.45 (s, 3H), 2.19-2.05 (m, 1H), 1.41 (s, 9H), 1.30 (t, 3H), 0.97-0.88 (m, 2H), −0.12 (s, 9H).

### Step B: ethyl 5-(3-{2-fluoro-4-[3-(methylamino)prop-1-yn-1-yl]phenoxy}propyl)-2-[methyl(5-methyl-6-{[(2Z)—3-{[2-(trimethylsilyl)ethoxy]methyl}-2,3-dihydro-1,3-benzothiazol-2-ylidene]amino}pyridazin-3-yl)amino]-1,3-thiazole-4-carboxylate

Trifluoroacetic acid (20 mL) was added to a stirred solution of the product from Step A (1.5 g, 1.71 mmol, 1 eq) in dichloromethane (60 mL) and the mixture was stirred at ambient temperature for 5 h. The reaction was diluted with dichloromethane, cooled to 0° C. and basified by the addition of 2N aqueous sodium hydroxide. The organic phase was dried (magnesium sulfate) and concentrated in vacuo. Purification by automated flash column chromatography (CombiFlash Rf, 40 g RediSep™ silica cartridge) eluting with a gradient of 0-10% methanol in dichloromethane afforded the desired product as a yellow gum (329 mg, 0.42 mmol, 25%). LC/MS (C₃₈H₄₆FN₇O₄SiS₂) 776 [M+H]⁺; RT 2.58 (LCMS-V-C). ¹H NMR (400 MHz, DMSO-d₆) δ 7.84 (dd, 1H), 7.67 (d, J=1.0 Hz, 1H), 7.49-7.40 (m, 2H), 7.31-7.22 (m, 2H), 7.21-7.11 (m, 2H), 5.86 (s, 2H), 4.26 (q, J=7.1 Hz, 2H), 4.15 (t, J=6.1 Hz, 2H), 3.76 (s, 3H), 3.76-3.67 (m, 2H), 3.45 (s, 2H), 3.33-3.22 (m, 2H), 2.46 (d, J=1.0 Hz, 3H), 2.30 (s, 3H), 2.18-2.06 (m, 2H), 1.29 (t, J=7.1 Hz, 3H), 0.97-0.88 (m, 2H), −0.11 (s, 9H).

### Preparation 6a_01: tert-Butyl N-[3-(3-fluoro-4-hydroxy-phenyl)prop-2-ynyl]-N-methyl-carbamate

Using Sonogashira General Procedure starting from 10.00 g of 2-fluoro-4-iodo-phenol (42.0 mmol, 1 eq.) as the appropriate phenol and 10.67 g of tert-butyl N-methyl-N-prop-2-ynyl-carbamate (63.1 mmol, 1.5 eq.) as alkyne reactant, 10.8 g (92%) of the desired product was obtained. ¹H NMR (500 MHz, DMSO-d₆) δ ppm 10.32 (s, 1H), 7.22 (brd, 1H), 7.08 (dm, 1H), 6.92 (dd, 1H), 4.21 (s, 2H), 2.85 (s, 3H), 1.41 (s, 9H); ¹³C NMR (125 MHz, DMSO-d₆) δ ppm 150.8, 146.4, 129.0, 119.6, 118.4, 113.2, 84.4, 82.7, 38.5, 33.8, 28.5; HRMS-ESI (m/z): [M−C₄H₈+H]⁺ calculated for C₁₁H₁₁FNO₃: 224.0717, found 224.0720.

### Preparation 6b_01: 4-[3-(Dimethylamino)prop-1-ynyl]-2-fluoro-phenol

Using Sonogashira General Procedure starting from 10.00 g of 2-fluoro-4-iodo-phenol (42.0 mmol, 1 eq.) as the appropriate phenol and 5.24 g of N,N-dimethylprop-2-yn-1-amine (63 mmol, 1.5 eq.) as alkyne reactant, 7.30 g (90%) of the desired product was obtained. ¹H NMR (500 MHz, DMSO-d₆) δ ppm 7.20 (dd, 1H), 7.07 (dm, 1H), 6.91 (m, 1H), 3.39 (m, 2H), 2.21 (m, 3H); ¹³C NMR (125 MHz, DMSO-d₆) δ ppm 150.9, 146.2, 128.9, 119.5, 118.4, 113.6, 84.5, 84.2, 48.2, 44.3; HRMS-ESI (m/z): [M+H]⁺ calculated for C₁₁H₁₃FNO: 194.0976, found 194.0981.

### Preparation 6f_01: 4-[3-(Dimethylamino)but-1-ynyl]-2-fluoro-phenol

### Step A: 4-(3-fluoro-4-triisopropylsilyloxy-phenyl)but-3-yn-2-ol

A 500 mL oven-dried, one-necked, round-bottomed flask equipped with a PTFE-coated magnetic stirring bar. It was charged with 4.76 g of 2-fluoro-4-iodo-phenol (20 mmol, 1 eq.) and 3.96 g of K₂CO₃ (40 mmol, 2 eq.) then 100 mL of dry MeCN was added. To the resulting mixture 5.13 mL of TIPSCl (4.62 g, 24 mmol, 1.2 eq.) was added dropwise near intensive stirring at rt. The resulting mixture was stirred at room temperature for 30 min, while the reaction reached complete conversion. The reaction mixture was filtered through a pad of Celite to remove the solid particles then to the filtrate 3.10 mL of but-3-yn-2-ol (2.81 g, 40 mmol, 2 eq.) and 20 ml of DIPA were added and placed under a nitrogen atmosphere through a gas inlet. After addition of 702 mg of Pd(PPh₃)₂Cl₂ (1 mmol, 0.05 eq.) and 190 mg of CuI (1 mmol, 0.05 eq.) the resulting mixture was stirred at room temperature for 30 min, while the reaction reached complete conversion. Celite was added to the reaction mixture and the volatiles were removed under reduced pressure. Then it was purified via flash column chromatography using heptane and EtOAc as eluents to give 6.2 g (92%) of the desired product as yellow oil. ¹H NMR (400 MHz, DMSO-d₆) δ ppm 7.26 (dd, 1H), 7.12 (dm, 1H), 6.98 (t, 1H), 5.44 (d, 1H), 4.55 (m, 1H), 1.36 (d, 3H), 1.24 (sp, 1H), 1.05 (d, 18H); ¹³C NMR (100 MHz, DMSO-d₆) δ ppm 153.2, 144.1, 128.8, 122.3, 119.6, 116.5, 93.4, 81.4, 57.1, 25.0, 18.0, 12.5; HRMS-ESI (m/z): [M+H]⁺ calculated for C₁₉H₃₀FO₂Si: 337.1994, found 337.1994.

### Step B: 4-(3-fluoro-4-triisopropylsilyloxy-phenyl)-N,N-dimethyl-but-3-yn-2-amine

Using Alkylation with in situ generated iodine General Procedure starting from 644 mg of the product from Step A (2 mmol, 1 eq.) as the appropriate alcohol and 5 mL of N-methylmethanamine (10 mmol, 5 eq., 2 M solution in MeOH), 360 mg (50%) of the desired product was obtained. ¹H NMR (500 MHz, DMSO-d₆) δ ppm 7.28 (dd, 1H), 7.14 (dm, 1H), 6.97 (t, 1H), 3.67 (q, 1H), 2.19 (s, 6H), 1.27 (d, 3H), 1.25 (m, 3H), 1.05 (d, 18H); ¹³C NMR (500 MHz, dmso-d₆) δ ppm 153.1, 144.0, 129.0, 122.3, 119.8, 116.6, 88.2, 84.1, 52.3, 41.3, 20.1, 18.0, 12.5; HRMS-ESI (m/z): [M+H]⁺ calculated for C₂₁H₃₅FNOSi: 364.2466, found 364.2470.

### Step C: 4-[3-(dimethylamino)but-1-ynyl]-2-fluoro-phenol

A 4 mL oven-dried vial equipped with a PTFE-coated magnetic stirring bar was charged with 200 mg of the product from Step B (0.55 mmol, 1 eq.) dissolved in 3.0 mL of dry THF, and then 660 μL of TBAF (1 M in THF, 0.66 mmol, 1.1 eq.) was added dropwise at rt. The resulting mixture was stirred at rt for 15 min, when the reaction reached complete conversion. The reaction mixture was quenched with the addition of 200 μL of cc. NH₄Cl, then Celite was added to the reaction mixture and the volatiles were removed under reduced pressure. Then it was purified via flash column chromatography using DCM and MeOH (1.2% NH₃) as eluents to give 80 mg (70%) of the desired product.

### Preparation 13_01: methyl 3-bromo-6-(methylamino)pyridine-2-carboxylate

### Step A: methyl 6-[bis(tert-butoxycarbonyl)amino]-3-bromo-pyridine-2-carboxylate

To methyl 6-amino-3-bromo-pyridine-2-carboxylate (25.0 g, 108.2 mmol) and DMAP (1.3 g, 0.1 eq) in DCM (541 mL) was added Boc₂O (59.0 g, 2.5 eq) at 0° C. and the reaction mixture was stirred for 2.5 h. After the addition of a saturated solution of NaHCO₃ and the extraction with DCM, the combined organic phases were dried and concentrated to get the desired product (45.0 g, 72.3%). LC/MS (C₁₇H₂₃BrN₂O₆Na) 453 [M+H]⁺.

### Step B: methyl 3-bromo-6-(tert-butoxycarbonylamino)pyridine-2-carboxylate

To the product from Step A (42.7 g, 74.34 mmol) in DCM (370 mL) was added TFA (17.1 mL, 3 eq) at 0° C. and the reaction mixture was stirred for 18 h. After washing with a saturated solution of NaHCO₃ and brine, the combined organic phases were dried, concentrated, and purified by column chromatography (silica gel, heptane and EtOAc as eluents) to give the desired product (28.3 g, 115.2%). ¹H NMR (400 MHz, DMSO-d₆): δ ppm 10.29 (s, 1H), 8.11 (d, 1H), 7.88 (d, 1H), 3.87 (s, 3H), 1.46 (s, 9H) ¹³C NMR (100 MHz, DMSO-d₆) δ ppm 165.6, 153.1, 151.8/148.3, 143.5, 116.3, 109.2, 53.2, 28.4. LC/MS (C₁₂H₁₅BrN₂O₄Na) 353 [M+H]⁺.

### Step C: methyl 3-bromo-6-[tert-butoxycarbonyl(methyl)amino]pyridine-2-carboxylate

To the product from Step B (2.96 g, 8.93 mmol) in acetone (45 mL) was added Cs₂CO₃ (8.7 g, 3 eq) and iodomethane (0.67 mL, 1.2 eq) and the reaction mixture was stirred for 3 h. After dilution with water and extraction with EtOAc, the combined organic phases were washed with brine, dried and concentrated to give the desired product (3.5 g, 112%). ¹H NMR (400 MHz, DMSO-d₆): δ ppm 8.13 (d, 1H), 7.78 (d, 1H), 3.90 (s, 3H), 3.27 (s, 3H), 1.47 (s, 9H); ¹³C NMR (100 MHz, DMSO-d₆) δ ppm 165.5, 153.6, 153.6, 147.5, 142.8, 122.5, 111.3, 82.0, 53.3, 34.3, 28.2; HRMS-ESI (m/z): [M+H]⁺ calculated for C₁₉H₁₈BrN₂O₄: 345.0450 found: 345.0429.

### Step D: methyl 3-bromo-6-(methylamino)pyridine-2-carboxylate

The product from Step C (3.0 g, 8.9 mmol) in 1,1,1,3,3,3-hexafluoroisopropanol (90 mL) was stirred at 100° C. for 18 h. Purification by column chromatography (silica gel, heptane and EtOAc as eluents) afforded the desired product (2.1 g, 96%). ¹H NMR (400 MHz, DMSO-d₆): δ ppm 7.63 (d, 1H), 7.04 (q, 1H), 6.53 (d, 1H), 3.83 (s, 3H), 2.73 (d, 3H); ¹³C NMR (100 MHz, DMSO-d₆) δ ppm 166.6, 158.2, 148.2, 141.3, 112.1, 101.3, 52.9, 28.3; HRMS-ESI (m/z): [M]⁺ calculated for C₈H₉BrN₂O₂: 243.9847 found: 243.9843.

### Preparation 14_01: methyl 3-[1-[[3,5-dimethyl-7-[2-(p-tolylsulfonyloxy)ethoxy]-1-adamantyl]methyl]-5-methyl-pyrazol-4-yl]-6-[methyl-[5-methyl-6-[(Z)-[3-(2-trimethylsilylethoxymethyl)-1,3-benzothiazol-2-ylidene]amino]pyridazin-3-yl]amino]pyridine-2-carboxylate

### Step A: methyl 3-[1-[[3-[2-[tert-butyl(diphenyl)silyl]oxyethoxy]-5,7-dimethyl-1-adamantyl]methyl]-5-methyl-pyrazol-4-yl]-6-(methylamino)pyridine-2-carboxylate

The mixture of the product from Preparation 13_01 (2.07 g, 8.45 mmol), the product from Preparation 7 (6.9 g, 1.2 eq), Cs₂CO₃ (8.26 g, 3 eq), and Pd(AtaPhos)₂Cl₂ (374 mg, 0.1 eq) in 1,4-dioxane (51 mL) and water (8.5 mL) was stirred at 80° C. for 1 h. Purification by column chromatography (silica gel, heptane and EtOAc as eluents) afforded the desired product (4.5 g, 74%). ¹H NMR (400 MHz, DMSO-d₆): δ ppm 7.66 (dm, 4H), 7.47-7.38 (m, 6H), 7.31 (d, 1H), 7.23 (s, 1H), 6.78 (q, 1H), 6.59 (d, 1H), 3.82 (s, 2H), 3.67 (t, 2H), 3.58 (s, 3H), 3.46 (t, 2H), 2.77 (d, 3H), 2.06 (s, 3H), 1.35 (s, 2H), 1.27/1.20 (d+d, 4H), 1.14/1.09 (d+d, 4H), 1.05/0.97 (d+d, 2H), 0.98 (s, 9H), 0.84 (s, 6H); ¹³C NMR (100 MHz, DMSO-d₆) δ ppm 140.1, 137.4, 135.6, 130.2/128.3, 109.8, 74.2, 64.4, 61.7, 58.9, 52.2, 50.0, 46.9, 46.0, 43.4, 39.8, 33.5, 30.1, 28.4, 27.1, 10.8; HRMS-ESI (m/z): [M+H]⁺ calculated for C₄₃H₅₇N₄O₄Si: 721.4149 found: 721.4148.

### Step B: methyl 3-[1-[[3-[2-[tert-butyl(diphenyl)silyl]oxyethoxy]-5,7-dimethyl-1-adamantyl]methyl]-5-methyl-pyrazol-4-yl]-6-[methyl-[5-methyl-6-[(Z)-[3-(2-trimethylsilylethoxymethyl)-1,3-benzothiazol-2-ylidene]amino]pyridazin-3-yl]amino]pyridine-2-carboxylate

Using Buchwald General Procedure III starting from the product from Step A at reflux for 18 h, 4.7 g (86%) of the desired product was obtained. ¹H NMR (400 MHz, DMSO-d₆): δ ppm 7.78 (dm, 1H), 7.69-7.36 (m, 10H), 7.63 (q, 1H), 7.63 (d, 1H), 7.47 (dm, 1H), 7.44 (m, 1H), 7.35 (s, 1H), 7.31 (d, 1H), 7.24 (m, 1H), 5.86 (s, 2H), 3.86 (s, 2H), 3.72 (m, 2H), 3.67 (t, 2H), 3.64 (s, 3H), 3.61 (s, 3H), 3.46 (t, 2H), 2.36 (d, 3H), 2.13 (s, 3H), 1.40-0.94 (m, 12H), 0.97 (s, 9H), 0.92 (m, 2H), 0.85 (s, 6H), −0.11 (s, 9H); HRMS-ESI (m/z): [M+H]⁺ calculated for C₆₁H₇₉N₈O₅SSi₂: 1091.5433 found: 1091.5426.

### Step C: methyl 3-[1-[[3-(2-hydroxyethoxy)-5,7-dimethyl-1-adamantyl]methyl]-5-methyl-pyrazol-4-yl]-6-[methyl-[5-methyl-6-[(Z)-[3-(2-trimethylsilylethoxymethyl)-1,3-benzothiazol-2-ylidene]amino]pyridazin-3-yl]amino]pyridine-2-carboxylate

To the product from Step B (1.0 g, 0.916 mmol) in THF (9 mL) was added a 1 M solution of TBAF in THF (1.0 mL, 1.1 eq) at 0° C. and the reaction mixture was stirred for 1 h. After quenching with a saturated solution of NH₄Cl and extraction with EtOAc, the combined organic phases were dried, concentrated, and purified by column chromatography (silica gel, DCM and MeOH as eluents) to give the desired product (752 mg, 96%). ¹H NMR (500 MHz, dmso-d₆) δ ppm 7.79 (dm, 1H), 7.66 (d, 1H), 7.64 (s, 1H), 7.47 (dm, 1H), 7.43 (m, 1H), 7.36 (s, 1H), 7.33 (d, 1H), 7.25 (m, 1H), 5.87 (s, 2H), 4.46 (t, 1H), 3.86 (s, 2H), 3.73 (m, 2H), 3.68 (s, 3H), 3.62 (s, 3H), 3.40 (m, 2H), 3.35 (t, 2H), 2.37 (s, 3H), 2.14 (s, 3H), 1.42-0.96 (m, 12H), 0.92 (m, 2H), 0.86 (s, 6H), −0.10 (s, 9H); HRMS-ESI (m/z): [M+H]⁺ calculated for C₄₅H₆₁N₈O₅SSi: 853.4255 found: 853.4256.

### Step D: methyl 3-[1-[[3,5-dimethyl-7-[2-(p-tolylsulfonyloxy)ethoxy]-1-adamantyl]methyl]-5-methyl-pyrazol-4-yl]-6-[methyl-[5-methyl-6-[(Z)-[3-(2-trimethylsilylethoxymethyl)-1,3-benzothiazol-2-ylidene]amino]pyridazin-3-yl]amino]pyridine-2-carboxylate

To the product from Step C (752 mg, 0.88 mmol) and triethylamine (0.5 mL, 4 eq) in DCM (4.4 mL) was added p-tolylsulfonyl-4-methylbenzenesulfonate (575.4 mg, 1.76 mmol, 2 eq) and the reaction mixture was stirred for 1 h. Purification by column chromatography (silica gel, heptane and EtOAc as eluents) afforded the desired product (722 mg, 81%). ¹H NMR (400 MHz, DMSO-d₆): δ ppm 7.79 (dm, 1H), 7.76 (dm, 2H), 7.68 (d, 1H), 7.64 (s, 1H), 7.47 (m, 1H), 7.46 (dm, 2H), 7.43 (td, 1H), 7.36 (s, 1H), 7.33 (d, 1H), 7.25 (td, 1H), 5.87 (s, 2H), 4.06 (m, 2H), 3.84 (s, 2H), 3.73 (t, 2H), 3.66 (s, 3H), 3.62 (s, 3H), 3.48 (m, 2H), 2.40 (s, 3H), 2.37 (s, 3H), 2.13 (s, 3H), 1.31-0.94 (m, 12H), 0.92 (t, 2H), 0.83 (s, 6H), −0.10 (s, 9H); ¹³C NMR (100 MHz, DMSO-d₆) δ ppm 141.2, 137.5, 130.6, 128.1, 127.2, 123.4, 123.4, 123.1, 114.7, 112.0, 72.9, 71.5, 66.7, 58.8, 58.4, 52.6, 36.6, 30.1, 21.6, 17.8, 17.4, 10.8, −0.9; HRMS-ESI (m/z): [M+H]⁺ calculated for C₅₂H₆₇N₈O₇S₂Si: 1007.4343 found: 1007.4344.

### Preparation of P1: 2-[3-(1,3-Benzothiazol-2-ylamino)-4-methyl-6,7-dihydro-5H-pyrido[2,3-c]pyridazin-8-yl]-5-[3-[4-[3-(dimethylamino)prop-1-ynyl]-2-fluoro-phenoxy]propyl]thiazole-4-carboxylic acid

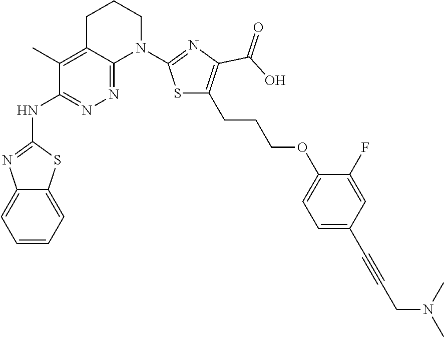 [SMILES: Cc1c(Nc2nc3ccccc3s2)nnc2c1CCCN2c1nc(C(=O)O)c(CCCOc2ccc(C#CCN(C)C)cc2F)s1]

Using Propargylic amine preparation General Procedure starting from Preparation 3d and dimethylamine as the appropriate amine. Then Hydrolysis General Procedure starting from the appropriate methyl ester, the desired product was obtained. HRMS-ESI (m/z): [M+H]⁺ calculated for C₃₄H₃₅FN₇O₃S₂: 672.2221, found 672.2205.

### Preparation of P2: 2-[[6-(1,3-Benzothiazol-2-ylamino)-5-methyl-pyridazin-3-yl]-methyl-amino]-5-[3-[4-[3-(dimethylamino)prop-1-ynyl]-2-fluoro-phenoxy]propyl]thiazole-4-carboxylic acid

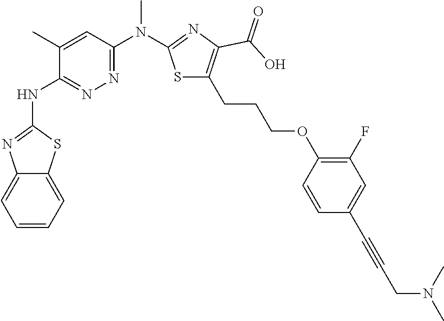 [SMILES: Cc1cc(N(C)c2nc(C(=O)O)c(CCCOc3ccc(C#CCN(C)C)cc3F)s2)nnc1Nc1nc2ccccc2s1]

### Step A: ethyl 5-[3-[4-[3-(dimethylamino)prop-1-ynyl]-2-fluoro-phenoxy]propyl]-2-[methyl-[5-methyl-6-[(Z)-[3-(2-trimethylsilylethoxymethyl)-1,3-benzothiazol-2-ylidene]amino]pyridazin-3-yl]amino]thiazole-4-carboxylate

Using Alkylation General Procedure starting from Preparation 5g_01 and Preparation 6b_01 as the appropriate phenol, the desired product was obtained. ¹H NMR (500 MHz, DMSO-d₆) δ ppm 7.84 (d, 1H), 7.67 (s, 1H), 7.47 (d, 1H), 7.44 (t, 1H), 7.33 (dd, 1H), 7.25 (t, 1H), 7.22 (dd, 1H), 7.16 (t, 1H), 5.86 (s, 2H), 4.26 (q, 2H), 4.15 (t, 2H), 3.77 (s, 3H), 3.72 (t, 2H), 3.49 (brs, 2H), 3.27 (t, 2H), 2.46 (s, 3H), 2.27 (s, 6H), 2.13 (qn, 2H), 1.29 (t, 3H), 0.92 (t, 2H), −0.11 (s, 9H); ¹³C NMR (125 MHz, DMSO-d₆) δ ppm 129.0, 127.2, 123.5, 123.2, 119.2, 117.7, 115.5, 111.9, 72.8, 68.5, 66.7, 60.7, 48.2, 44.0, 35.3, 31.1, 23.2, 17.9, 17.8, 14.6, −0.9; HRMS-ESI (m/z): [M+H]⁺ calculated for C₃₉H₄₉FN₇O₄S₂Si: 790.3035, found 790.3023.

### Step B: 2-[[6-(1,3-benzothiazol-2-ylamino)-5-methyl-pyridazin-3-yl]-methyl-amino]-5-[3-[4-[3-(dimethylamino)prop-1-ynyl]-2-fluoro-phenoxy]propyl]thiazole-4-carboxylic acid

Using Deprotection and Hydrolysis General Procedure starting from the product from Step A as the appropriate ethyl ester, the desired product was obtained. HRMS-ESI (m/z): [M+H]⁺ calculated for C₃₁H₃₁FN₇O₃S₂: 632.1908, found 632.1913.

### Preparation of P3: 2-{3-[(1,3-Benzothiazol-2-yl)amino]-4-methyl-5H,6H,7H,8H-pyrido[2,3-c]pyridazin-8-yl}-5-(3-{2-fluoro-4-[3-(methylamino)prop-1-yn-1-yl]phenoxy}propyl)-1,3-thiazole-4-carboxylic acid

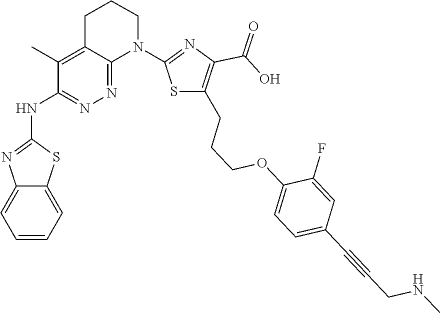 [SMILES: CNCC#Cc1ccc(OCCCc2sc(N3CCCc4c3nnc(Nc3nc5ccccc5s3)c4C)nc2C(=O)O)c(F)c1]

### Step A: ethyl 5-{3-[4-(3-{[(tert-butoxy)carbonyl](methyl)amino}prop-1-yn-1-yl)-2-fluorophenoxy]propyl}-2-(4-methyl-3-{[(2Z)-3-{[2-(trimethylsilyl)ethoxy]methyl}-2,3-dihydro-1,3-benzothiazol-2-ylidene]amino}-5H,6H,7H,8H-pyrido[2,3-c]pyridazin-8-yl)-1,3-thiazole-4-carboxylate

To a solution of the product from Preparation 3g (500 mg, 0.78 mmol, 1 eq) in toluene (15 mL) was added the product from Preparation 4c (327 mg, 1.17 mmol, 1.5 eq), followed by triphenylphosphine (307 mg, 1.17 mmol, 1.5 eq) and diisopropyl azodicarboxylate (230 μL, 1.17 mmol, 1.5 eq) and he mixture was heated at reflux overnight. The reaction was partitioned between dichloromethane and water, and the organic phase was dried (PTFE phase separator) and concentrated in vacuo. Purification by automated flash column chromatography (CombiFlash Rf, 24 g RediSep™ silica cartridge) eluting with a gradient of 0-50% ethyl acetate in iso-heptane afforded the desired product as an off-white foam (715 mg, 0.79 mmol, >100%). LC/MS (C₄₅H₅₆FN₇O₆SiS₂) 902 [M+H]⁺; RT 1.46 (LCMS-V-B2). ¹H NMR (400 MHz, DMSO-d₆) δ 7.82 (dt, J=7.6, 0.9 Hz, 1H), 7.48-7.37 (m, 2H), 7.33 (d, J=11.6 Hz, 1H), 7.28-7.13 (m, 3H), 5.84 (s, 2H), 4.32-4.17 (m, 6H), 4.15 (t, J=6.1 Hz, 2H), 3.72 (dd, J=8.5, 7.4 Hz, 2H), 3.27 (d, J=15.4 Hz, 2H), 2.93-2.75 (m, 5H), 2.36 (s, 3H), 2.19-2.10 (m, 2H), 2.10-1.98 (m, 2H), 1.40 (s, 9H), 1.28 (t, 3H), 0.96-0.89 (m, 2H), −0.11 (s, 9H).

### Step B: ethyl 2-{3-[(1,3-benzothiazol-2-yl)amino]-4-methyl-5H,6H,7H,8H-pyrido[2,3-c]pyridazin-8-yl}-5-(3-{2-fluoro-4-[3-(methylamino)prop-1-yn-1-yl]phenoxy}propyl)-1,3-thiazole-4-carboxylate

To a solution of the product from Step A (1.67 g, 1.85 mmol, 1 eq) in acetonitrile (17 mL) was added hydrogen fluoride-pyridine (3.22 mL, 37 mmol, 20 eq) and the mixture was heated at 60° C. for 2 h. The reaction was partitioned between 3:1 dichloromethane/isopropanol and 2N aqueous sodium hydroxide, and the organic phase was washed with brine, dried (PTFE phase separator) and concentrated in vacuo. Purification by automated flash column chromatography (CombiFlash Rf, 80 g RediSep™ silica cartridge) eluting with a gradient of 0-7% methanol in dichloromethane afforded the desired product as a yellow solid (1.02 g, 1.52 mmol, 82%). LC/MS (C₃₄H₃₄FN₇O₃S₂) 672 [M+H]⁺; RT 2.06 (LCMS-V-C). ¹H NMR (400 MHz, DMSO-d₆) δ 7.89 (dd, J=7.8, 1.2 Hz, 1H), 7.50 (d, J=8.1 Hz, 1H), 7.38 (ddd, J=8.2, 7.3, 1.2 Hz, 1H), 7.32-7.25 (m, 1H), 7.23-7.12 (m, 3H), 4.32-4.21 (m, 4H), 4.15 (t, J=6.1 Hz, 2H), 3.45 (s, 2H), 3.32-3.23 (m, 2H), 2.89 (t, J=6.4 Hz, 2H), 2.35 (s, 3H), 2.31 (s, 3H), 2.20-2.10 (m, 2H), 2.09-1.97 (m, 2H), 1.30 (t, J=7.1 Hz, 3H).

### Step C: 2-{3-[(1,3-benzothiazol-2-yl)amino]-4-methyl-5H,6H,7H,8H-pyrido[2,3-c]pyridazin-8-yl}-5-(3-{2-fluoro-4-[3-(methylamino)prop-1-yn-1-yl]phenoxy}propyl)-1,3-thiazole-4-carboxylic acid

To a solution of the product from Step B (1.02 g, 1.52 mmol, 1 eq) in 1,4-dioxane (50 mL) was lithium hydroxide monohydrate (637 mg, 15.2 mmol, 10 eq) and the mixture was heated at 110° C. overnight. Purification by automated flash column chromatography (CombiFlash Rf, 80 g RediSep™ silica cartridge) eluting with a gradient of 0-70% 0.7N methanolic ammonia in dichloromethane gave a solid that was triturated with acetonitrile, filtered and dried under vacuum to afford the desired product as a yellow solid (657 mg, 1.02 mmol, 67%). HRMS-ESI (m/z) [M+H]⁺ calculated for C₃₂H₃₁FN₇O₃S₂: 644.1914, found 644.1930.

### Preparation of P4: 3-[[5-[[6-(1,3-Benzothiazol-2-ylamino)-5-methyl-pyridazin-3-yl]-[4-carboxy-5-[3-[2-fluoro-4-[3-(methylamino)prop-1-ynyl]phenoxy]propyl]thiazol-2-yl]amino]-2-hydroxy-pentyl]-dimethyl-ammonio]propane-1-sulfonate

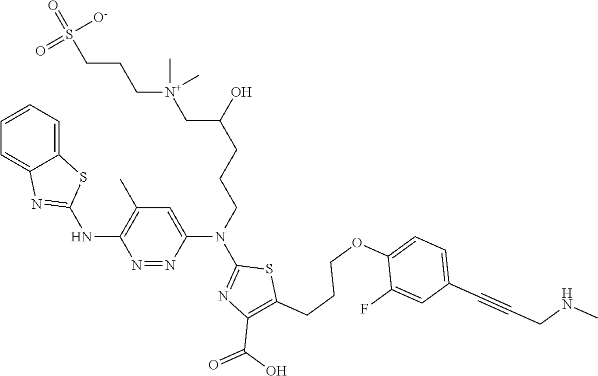 [SMILES: CNCC#Cc1ccc(OCCCc2sc(N(CCCC(O)C[N+](C)(C)CCCS(=O)(=O)[O-])c3cc(C)c(Nc4nc5ccccc5s4)nn3)nc2C(=O)O)c(F)c1]

### Step A: methyl 5-[3-[4-[3-[tert-butoxycarbonyl(methyl)amino]prop-1-ynyl]-2-fluoro-phenoxy]propyl]-2-[[4-[tert-butyl(diphenyl)silyl]oxy-5-(dimethylamino)pentyl]-[5-methyl-6-[(Z)-[3-(2-trimethylsilylethoxymethyl)-1,3-benzothiazol-2-ylidene]amino]pyridazin-3-yl]amino]thiazole-4-carboxylate

Using Alkylation with tosylate General Procedure starting from Preparation 5a_01 and N-methylmethanamine as the appropriate amine, the desired product was obtained. HRMS-ESI (m/z): [M+H]⁺ calculated for C₆₄H₈₄FN₈O₇S₂Si₂: 1215.5421, found 1215.5389.

### Step B: 3-[[5-[[6-(1,3-Benzothiazol-2-ylamino)-5-methyl-pyridazin-3-yl]-[4-carboxy-5-[3-[2-fluoro-4-[3-(methylamino)prop-1-ynyl]phenoxy]propyl]thiazol-2-yl]amino]-2-hydroxy-pentyl]-dimethyl-ammonio]propane-1-sulfonate

The product from Step A was suspended in MeCN (5 mL/mmol) then oxathiolane 2,2-dioxide (10 eq.) was added and stirred at 60° C. for on (full conversion was observed). The reaction mixture was concentrated. The crude mixture which contained 3-[[5-[[5-[3-[4-[3-[tert-butoxycarbonyl(methyl)amino]prop-1-ynyl]-2-fluoro-phenoxy]propyl]-4-methoxycarbonyl-thiazol-2-yl]-[5-methyl-6-[(Z)-[3-(2-trimethylsilylethoxymethyl)-1,3-benzothiazol-2-ylidene]amino]pyridazin-3-yl]amino]-2-[tert-butyl(diphenyl)silyl]oxy-pentyl]-dimethyl-ammonio]propane-1-sulfonate (LC-MS-ESI (m/z): [M+H]⁺ calculated for C₆₇H₉₀FN₈O₁₀S₃Si₂: 1337.5, found 1337.6) was transferred directly to the next reaction using Quaternary salt deprotection General Procedure, to afford the desired product. HRMS-ESI (m/z): [M+H]⁺ calculated for C₃₉H₄₈FN₈O₇S₃: 855.2787, found 855.2786.

### Preparation of P5: 2-[[6-(1,3-Benzothiazol-2-ylamino)-5-methyl-pyridazin-3-yl]-[4-hydroxy-5-(trimethylammonio)pentyl]amino]-5-[3-[2-fluoro-4-[3-(methylamino)prop-1-ynyl]phenoxy]propyl]thiazole-4-carboxylate

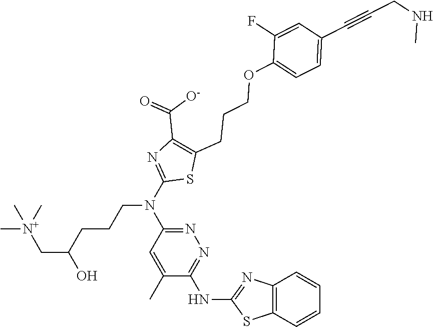 [SMILES: CNCC#Cc1ccc(OCCCc2sc(N(CCCC(O)C[N+](C)(C)C)c3cc(C)c(Nc4nc5ccccc5s4)nn3)nc2C(=O)[O-])c(F)c1]

### Step A: methyl 5-[3-[4-[3-[tert-butoxycarbonyl(methyl)amino]prop-1-ynyl]-2-fluoro-phenoxy]propyl]-2-[[4-[tert-butyl(diphenyl)silyl]oxy-5-(dimethylamino)pentyl]-[5-methyl-6-[(Z)-[3-(2-trimethylsilylethoxymethyl)-1,3-benzothiazol-2-ylidene]amino]pyridazin-3-yl]amino]thiazole-4-carboxylate

Using Alkylation with tosylate General Procedure starting from Preparation 5a_01 and N-methylmethanamine as the appropriate amine, the desired product was obtained. HRMS-ESI (m/z): [M+H]⁺ calculated for C₆₄H₈₄FN₈O₇S₂Si₂: 1215.5421, found 1215.5389.

### Step B: 2-[[6-(1,3-benzothiazol-2-ylamino)-5-methyl-pyridazin-3-yl]-[4-hydroxy-5-(trimethylammonio)pentyl]amino]-5-[3-[2-fluoro-4-[3-(methylamino)prop-1-ynyl]phenoxy]propyl]thiazole-4-carboxylate

The product from Step A was dissolved in the mixture of acetonitrile (4 mL/mmol) and N,N-dimethylformamide (1 mL/mmol) then iodomethane (5 eq.) was added and stirred at rt until full conversion was observed (ca. 1 h). The reaction mixture was concentrated. The crude mixture which contained [5-[[5-[3-[4-[3-[tert-butoxycarbonyl(methyl)amino]prop-1-ynyl]-2-fluoro-phenoxy]propyl]-4-methoxycarbonyl-thiazol-2-yl]-[5-methyl-6-[(Z)-[3-(2-trimethylsilylethoxymethyl)-1,3-benzothiazol-2-ylidene]amino]pyridazin-3-yl]amino]-2-[tert-butyl(diphenyl)silyl]oxy-pentyl]-trimethyl-ammonium (LC-MS-ESI (m/z): [M]⁺ calculated for C₆₅H₈₆FN₈O₇S₂Si₂: 1229.6, found 1229.4) was transferred to the next reaction using Quaternary salt deprotection General Procedure, to afford the desired product. HRMS-ESI (m/z): [M+H]⁺ calculated for C₃₇H₄₄FN₈O₄S₂: 747.2905, found 747.2900.

### Preparation of P6: 2-[[6-(1,3-Benzothiazol-2-ylamino)-5-methyl-pyridazin-3-yl]-[3-(dimethylamino)propyl]amino]-5-[3-[2-fluoro-4-[3-(methylamino)prop-1-ynyl]phenoxy]propyl]thiazole-4-carboxylic acid

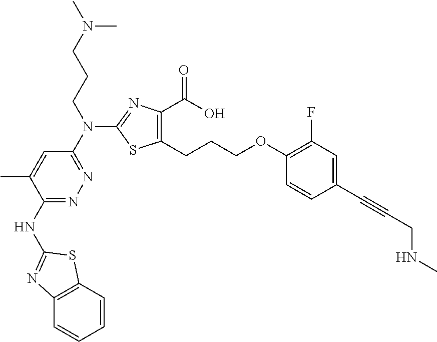

### Step A: methyl 2-[tert-butoxycarbonyl-[3-(dimethylamino)propyl]amino]-5-[3-[4-[3-[tert-butoxycarbonyl(methyl)amino]prop-1-ynyl]-2-fluoro-phenoxy]propyl]thiazole-4-carboxylate

Using Mitsunobu General Procedure II starting from Preparation 1b_01 and 3-(dimethylamino)propan-1-ol, 1.40 g (quant., the sample contained approx. 35 n/n % DIAD-2H) of the desired product was produced. ¹H NMR (400 MHz, DMSO-d₆) δ ppm 7.30 (dd, 1H), 7.21 (dm, 1H), 7.13 (t, 1H), 4.23 (s, 2H), 4.10 (t, 2H), 4.01 (t, 2H), 3.74 (s, 3H), 3.22 (t, 2H), 2.86 (s, 3H), 2.24 (t, 2H), 2.12 (s, 6H), 2.08 (m, 2H), 1.74 (m, 2H), 1.51/1.41 (s, 18H); HRMS-ESI (m/z): [M+H]⁺ calculated for C₃₃H₄₈FN₄O₇S: 663.3228, found 663.3218.

### Step B: methyl 5-[3-[4-[3-[tert-butoxycarbonyl(methyl)amino]prop-1-ynyl]-2-fluoro-phenoxy]propyl]-2-[3-(dimethylamino)propylamino]thiazole-4-carboxylate

Using Deprotection with HFIP General Procedure starting from the product from Step A, 0.95 g (80%) of the desired product was produced. ¹H NMR (500 MHz, DMSO-d₆) δ ppm 7.57 (t, 1H), 7.31 (d, 1H), 7.21 (d, 1H), 7.13 (t, 1H), 4.23 (br., 2H), 4.07 (t, 2H), 3.69 (s, 3H), 3.17 (q, 2H), 3.12 (t, 2H), 2.86 (br., 3H), 2.24 (t, 2H), 2.11 (s, 6H), 2.00 (quint., 2H), 1.63 (m, 2H), 1.41 (s, 9H); ¹³C NMR (125 MHz, DMSO-d₆) δ ppm 129.1, 119.3, 115.4, 68, 57.0, 51.7, 45.6, 42.8, 38.6, 33.8, 30.6, 28.5, 27.0, 23.3; HRMS-ESI (m/z): [M+H]⁺ calculated for C₂₈H₄₀FN₄O₅S: 563.2703, found 563.2694.

### Step C: methyl 5-[3-[4-[3-[tert-butoxycarbonyl(methyl)amino]prop-1-ynyl]-2-fluoro-phenoxy]propyl]-2-[3-(dimethylamino)propyl-[5-methyl-6-[(Z)-[3-(2-trimethylsilylethoxymethyl)-1,3-benzothiazol-2-ylidene]amino]pyridazin-3-yl]amino]thiazole-4-carboxylate

Using Buchwald General Procedure III starting from the product from Step B and Preparation 4a_01, 0.79 g (51%) of the desired product was produced. ¹H NMR (500 MHz, DMSO-d₆) δ ppm 7.84 (d, 1H), 7.73 (s, 1H), 7.46 (dd, 1H), 7.43 (td, 1H), 7.31 (brd., 1H), 7.25 (td, 1H), 7.21 (d, 1H), 7.16 (t, 1H), 5.86 (s, 2H), 4.35 (t, 2H), 4.20 (br., 2H), 4.15 (t, 2H), 3.76 (s, 3H), 3.72 (t, 2H), 3.27 (t, 2H), 2.84 (br., 3H), 2.45 (s, 3H), 2.32 (t, 2H), 2.18 (s, 6H), 2.13 (m, 2H), 1.86 (m, 2H), 1.40 (s, 9H), 0.92 (t, 2H), −0.11 (s, 9H); ¹³C NMR (125 MHz, DMSO-d₆) δ ppm 129.1, 127.2, 123.4, 123.2, 119.3, 117.6, 115.4, 111.9, 72.8, 68.4, 66.7, 56.4, 51.9, 45.7, 45.5, 38.5, 33.8, 31.0, 28.5, 25.0, 23.1, 17.9, 17.8, −1.0; HRMS-ESI (m/z): [M+H]⁺ calculated for C₄₆H₆₂FN₈O₆S₂Si: 933.3987, found 933.3990.

### Step D: 2-[[6-(1,3-benzothiazol-2-ylamino)-5-methyl-pyridazin-3-yl]-[3-(dimethylamino)propyl]amino]-5-[3-[2-fluoro-4-[3-(methylamino)prop-1-ynyl]phenoxy]propyl]thiazole-4-carboxylic acid

Using Deprotection and Hydrolysis General Procedure followed by repurification via reverse phase preparative chromatography (C18, 0.1% TFA in water:MeCN) starting from the product from Step C, the TFA-salt of the desired product was obtained. HRMS-ESI (m/z): [M+2H]²⁺ calculated for C₃₄H₃₉FN₈O₃S₂: 345.1280, found 345.1265.

### Preparation of P7: 2-({6-[(1,3-Benzothiazol-2-yl)amino]-5-methylpyridazin-3-yl}(methyl)amino)-5-(3-{2-fluoro-4-[3-(methylamino)prop-1-yn-1-yl]phenoxy}propyl)-1,3-thiazole-4-carboxylic acid

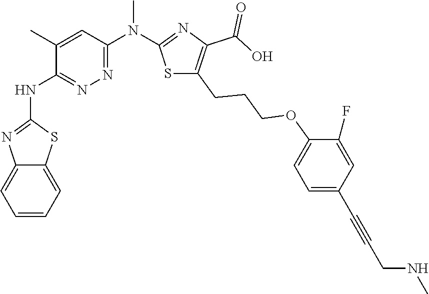 [SMILES: CNCC#Cc1ccc(OCCCc2sc(N(C)c3cc(C)c(Nc4nc5ccccc5s4)nn3)nc2C(=O)O)c(F)c1]

### Step A: ethyl 2-({6-[(1,3-benzothiazol-2-yl)amino]-5-methylpyridazin-3-yl}(methyl)amino)-5-(3-{2-fluoro-4-[3-(methylamino)prop-1-yn-1-yl]phenoxy}propyl)-1,3-thiazole-4-carboxylate

Trifluoroacetic acid (20 mL) was added to a stirred solution of the product from Preparation 5j_01, Step A (1.5 g, 1.71 mmol, 1 eq) in dichloromethane (60 mL) and the mixture was stirred at ambient temperature overnight. The reaction was diluted with dichloromethane, cooled to 0° C. then basified by the addition of 2N aqueous sodium hydroxide, and the organic phase was dried (magnesium sulfate) and concentrated in vacuo. Purification by automated flash column chromatography (CombiFlash Rf, 40 g RediSep™ silica cartridge) eluting with a gradient of 0-10% methanol in dichloromethane afforded the desired product as a yellow solid (361 mg, 0.56 mmol, 33%). LC/MS (C₃₂H₃₂FN₇O₃S₂) 646 [M+H]⁺; RT 1.98 (LCMS-V-C). ¹H NMR (400 MHz, DMSO-d₆) δ 7.91 (d, 1H), 7.68 (d, J=1.2 Hz, 1H), 7.53 (d, J=7.9 Hz, 1H), 7.39 (ddd, J=8.2, 7.2, 1.3 Hz, 1H), 7.32-7.11 (m, 4H), 4.25 (q, J=7.1 Hz, 2H), 4.15 (t, J=6.2 Hz, 2H), 3.77 (s, 3H), 3.46 (s, 2H), 3.27 (t, J=7.7 Hz, 2H), 2.47 (d, J=1.0 Hz, 3H), 2.31 (s, 3H), 2.19-2.07 (m, 2H), 2.23 (s, 1H), 1.30 (t, J=7.1 Hz, 3H).

### Step B: 2-({6-[(1,3-benzothiazol-2-yl)amino]-5-methylpyridazin-3-yl}(methyl)amino)-5-(3-{2-fluoro-4-[3-(methylamino)prop-1-yn-1-yl]phenoxy}propyl)-1,3-thiazole-4-carboxylic acid

To a solution of the product from Step B (361 mg, 0.56 mmol, 1 eq) in 1,4-dioxane (15 mL) was added lithium hydroxide monohydrate (352 mg, 8.39 mmol, 15 eq) and the mixture was heated at 100° C. overnight. The reaction was allowed to cool to ambient temperature and concentrated in vacuo. The residue was triturated with water, filtered, washed with water then diethyl ether, and dried under vacuum to afford the desired product as a yellow solid (286 mg, 0.46 mmol, 83%) [as a lithium salt]. HRMS-ESI (m/z) [M+H]⁺ calculated for C₃₀H₂₉FN₇O₃S₂: 618.1752, found 618.1767.

### Preparation of P8: 2-[3-(1,3-Benzothiazol-2-ylamino)-4-methyl-6,7-dihydro-5H-pyrido[2,3-c]pyridazin-8-yl]-5-[3-[2-fluoro-4-[3-(4-methylpiperazin-1-yl) but-1-ynyl]phenoxy]propyl]thiazole-4-carboxylic acid

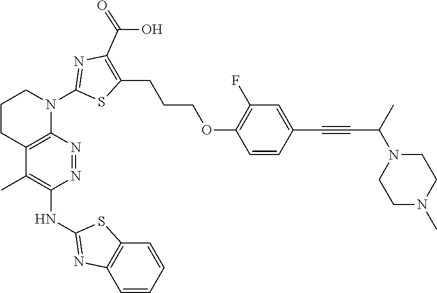 [SMILES: Cc1c(Nc2nc3ccccc3s2)nnc2c1CCCN2c1nc(C(=O)O)c(CCCOc2ccc(C#CC(C)N3CCN(C)CC3)cc2F)s1]

### Step A: methyl 2-(3-chloro-4-methyl-6,7-dihydro-5H-pyrido[2,3-c]pyridazin-8-yl)-5-[3-[2-fluoro-4-[3-(4-methylpiperazin-1-yl) but-1-ynyl]phenoxy]propyl]thiazole-4-carboxylate

A 24 mL oven-dried vial was equipped with a PTFE-coated magnetic stirring bar, and was charged with 250 mg 1-methylpiperazine (2.5 mmol, 5.0 eq.) dissolved in 2.5 mL dry THF Then 133 mg 3-bromobut-1-yne (1.0 mmol, 2.0 equiv) was added dropwise via syringe over a period of 5 minutes, and stirred at that temperature for 30 min. To the resulting mixture 301 mg of Preparation 3a (0.50 mmol, 1.0 eq.), 18.15 mg Pd(PPh₃)₂Cl₂ (0.025 mmol, 0.05 eq.) and 4.76 CuI (0.025 mmol, 0.05 eq.) were added, then it was heated to 60° C. and stirred for 2 h at that temperature. The reaction reached complete conversion. Celite was added to the reaction mixture and the volatiles were removed under reduced pressure. Then it was purified via flash chromatography using DCM and MeOH (1.2% NH₃) as eluents to give 300 mg (95% Yield) of the desired product.

### Step B: methyl 2-[3-(1,3-benzothiazol-2-ylamino)-4-methyl-6,7-dihydro-5H-pyrido[2,3-c]pyridazin-8-yl]-5-[3-[2-fluoro-4-[3-(4-methylpiperazin-1-yl) but-1-ynyl]phenoxy]propyl]thiazole-4-carboxylate

Using Buchwald General Procedure II starting from 300 mg of the product from Step A (0.47 mmol, 1.0 eq.) and 140 mg 1,3-benzothiazol-2-amine (0.94 mmol, 2.0 eq.), 150 mg (42%) mg of the desired product was obtained.

### Step C: 2-[3-(1,3-benzothiazol-2-ylamino)-4-methyl-6,7-dihydro-5H-pyrido[2,3-c]pyridazin-8-yl]-5-[3-[2-fluoro-4-[3-(4-methylpiperazin-1-yl) but-1-ynyl]phenoxy]propyl]thiazole-4-carboxylic acid

Using Hydrolysis General Procedure starting from the product from Step B as the appropriate methyl ester, the desired product was obtained. ¹H NMR (500 MHz, DMSO-d₆) δ ppm 7.87 (d, 1H), 7.49 (d, 1H), 7.36 (t, 1H), 7.26 (dd, 1H), 7.2 (t, 1H), 7.16 (dd, 1H), 7.13 (t, 1H), 4.27 (t, 2H), 4.12 (t, 2H), 3.65 (q, 1H), 3.27 (t, 2H), 2.87 (t, 2H), 2.62-2.21 (brm, 8H), 2.14 (s, 3H), 2.13 (qn, 2H), 2.04 (qn, 2H), 1.33 (s, 3H), 1.25 (d, 3H); ¹³C NMR (125 MHz, DMSO-d₆) δ ppm 164.3, 155.4, 151.5, 151.4, 148.6, 147.2, 145.1, 140.2, 136.3, 130.2, 129.0, 129.0, 127.6, 126.5, 122.5, 122.3, 119.2, 116.4, 115.5, 115.4, 88.4, 84.1, 68.5, 51.7, 46.3, 46.1, 31, 23.9, 23.0, 20.3, 19.6, 12.9; HRMS-ESI (m/z) [M+H]⁺ calculated for C₃₇H₄₀FN₈O₃S₂: 727.2649, found 727.2630

### Preparation of P9: 2-[3-(1,3-Benzothiazol-2-ylamino)-4-methyl-6,7-dihydro-5H-pyrido[2,3-c]pyridazin-8-yl]-5-[3-[2-fluoro-4-(3-pyrrolidin-1-ylprop-1-ynyl)phenoxy]propyl]thiazole-4-carboxylic acid

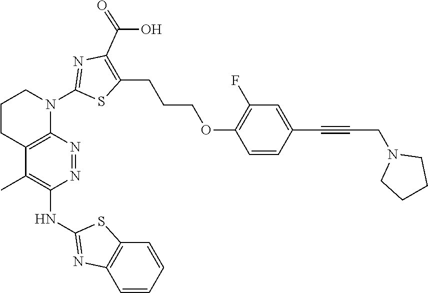 [SMILES: Cc1c(Nc2nc3ccccc3s2)nnc2c1CCCN2c1nc(C(=O)O)c(CCCOc2ccc(C#CCN3CCCC3)cc2F)s1]

### Step A: methyl 2-[3-(1,3-benzothiazol-2-ylamino)-4-methyl-6,7-dihydro-5H-pyrido[2,3-c]pyridazin-8-yl]-5-[3-[2-fluoro-4-(3-pyrrolidin-1-ylprop-1-ynyl)phenoxy]propyl]thiazole-4-carboxylate

Using Propargylic amine preparation General Procedure starting from 258 mg of Preparation 3d (0.40 mmol, 1 eq.) as the appropriate propargylic alcohol and pyrrolidine (20 eq, 670 mg), 120 mg of the desired product (43%) was obtained.

### Step B: 2-[3-(1,3-benzothiazol-2-ylamino)-4-methyl-6,7-dihydro-5H-pyrido[2,3-c]pyridazin-8-yl]-5-[3-[2-fluoro-4-(3-pyrrolidin-1-ylprop-1-ynyl)phenoxy]propyl]thiazole-4-carboxylic acid

Using Hydrolysis General Procedure starting from the product from Step A as the appropriate methyl ester, the desired product was obtained. ¹H NMR (500 MHz, DMSO-d₆) δ ppm 7.88 (d, 1H), 7.49 (d, 1H), 7.37 (t, 1H), 7.29 (dd, 1H), 7.2 (dd, 1H), 7.19 (t, 1H), 7.14 (t, 1H), 4.27 (t, 2H), 4.14 (t, 2H), 3.52 (s, 2H), 3.27 (t, 2H), 2.88 (t, 2H), 2.52 (t, 4H), 2.34 (s, 3H), 2.13 (qn, 2H), 2.04 (qn, 2H), 1.69 (t, 4H); ¹³C NMR (125 MHz, DMSO-d₆) δ ppm 151.5, 151.4, 148.6, 147.3, 145.1, 140.1, 136.7, 130.2, 129.0, 129.0, 127.5, 126.5, 122.5, 122.3, 119.2, 116.5, 115.5, 115.4, 85.9, 83.3, 68.6, 52.3, 46.3, 43.3, 31.1, 23.8, 23.8, 23.0, 20.4, 12.9; HRMS-ESI (m/z): [M+H]⁺ calculated for C₃₅H₃₅FN₇O₃S₂: 684.2221, found 684.2209.

### Preparation of P10: 2-[3-(1,3-Benzothiazol-2-ylamino)-4-methyl-6,7-dihydro-5H-pyrido[2,3-c]pyridazin-8-yl]-5-[3-[2-fluoro-4-[3-(4-methylpiperazin-1-yl)prop-1-ynyl]phenoxy]propyl]thiazole-4-carboxylic acid

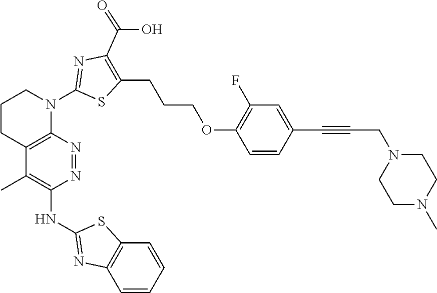 [SMILES: Cc1c(Nc2nc3ccccc3s2)nnc2c1CCCN2c1nc(C(=O)O)c(CCCOc2ccc(C#CCN3CCN(C)CC3)cc2F)s1]

### Step A: methyl 2-[3-(1,3-benzothiazol-2-ylamino)-4-methyl-6,7-dihydro-5H-pyrido[2,3-c]pyridazin-8-yl]-5-[3-[2-fluoro-4-[3-(4-methylpiperazin-1-yl)prop-1-ynyl]phenoxy]propyl]thiazole-4-carboxylate

Using Propargylic amine preparation General Procedure starting from 100 mg of Preparation 3d (0.155 mmol, 1 eq.) as the appropriate propargylic alcohol and 1-methylpiperazine (310.7 mg, 20 eq.), 150 mg of the desired product (79%) was obtained.

### Step B: 2-[3-(1,3-benzothiazol-2-ylamino)-4-methyl-6,7-dihydro-5H-pyrido[2,3-c]pyridazin-8-yl]-5-[3-[2-fluoro-4-[3-(4-methylpiperazin-1-yl)prop-1-ynyl]phenoxy]propyl]thiazole-4-carboxylic acid

Using Hydrolysis General Procedure starting from the product from Step A as the appropriate methyl ester, the desired product was obtained. HRMS-ESI (m/z): [M+H]⁺ calculated for C₃₆H₃₈FN₈O₃S₂: 713.2486, found 713.2474.

### Preparation of P11: 2-[[6-(1,3-Benzothiazol-2-ylamino)-5-methyl-pyridazin-3-yl]-methyl-amino]-5-[3-[4-[3-(dimethylamino) but-1-ynyl]-2-fluoro-phenoxy]propyl]thiazole-4-carboxylic acid

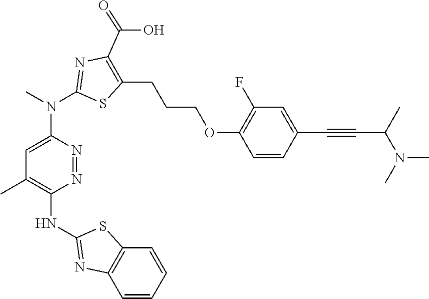

### Step A: ethyl 5-[3-[4-[3-(dimethylamino) but-1-ynyl]-2-fluoro-phenoxy]propyl]-2-[methyl-[5-methyl-6-[(Z)-[3-(2-trimethylsilylethoxymethyl)-1,3-benzothiazol-2-ylidene]amino]pyridazin-3-yl]amino]thiazole-4-carboxylate

Using Alkylation General Procedure starting from Preparation 5g_01 and Preparation 6f_01 as the appropriate phenol, the desired product was obtained.

### Step B: 2-[[6-(1,3-benzothiazol-2-ylamino)-5-methyl-pyridazin-3-yl]-methyl-amino]-5-[3-[4-[3-(dimethylamino) but-1-ynyl]-2-fluoro-phenoxy]propyl]thiazole-4-carboxylic acid

Using Deprotection and Hydrolysis General Procedure starting from the product from Step A as the appropriate ethyl ester, the desired product was obtained. HRMS-ESI (m/z): [M+H]⁺ calculated for C₃₂H₃₃FN₇O₃S₂: 646.2065, found 646.2057.

### Preparation of P12: 2-[3-(1,3-Benzothiazol-2-ylamino)-4-methyl-6,7-dihydro-5H-pyrido[2,3-c]pyridazin-8-yl]-5-[3-[4-[3-[2-(dimethylamino)ethylamino]prop-1-ynyl]-2-fluoro-phenoxy]propyl]thiazole-4-carboxylic acid

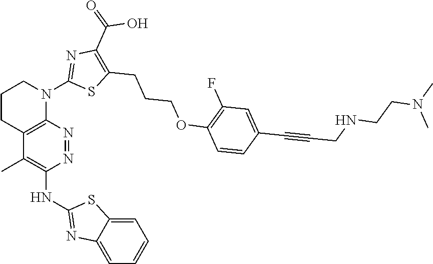 [SMILES: Cc1c(Nc2nc3ccccc3s2)nnc2c1CCCN2c1nc(C(=O)O)c(CCCOc2ccc(C#CCNCCN(C)C)cc2F)s1]

### Step A: methyl 5-[3-[4-[3-[tert-butoxycarbonyl-[2-(dimethylamino)ethyl]amino]prop-1-ynyl]-2-fluoro-phenoxy]propyl]-2-(3-chloro-4-methyl-6,7-dihydro-5H-pyrido[2,3-c]pyridazin-8-yl)thiazole-4-carboxylate

Using Sonogashira General Procedure starting from 1.00 g of Preparation 3a (1.66 mmol, 1 eq.) and 413 mg of tert-butyl N-[2-(dimethylamino)ethyl]-N-prop-2-ynyl-carbamate (1.83 mmol, 1.1 eq.) as the appropriate alkyne, the desired product was isolated as yellow solid. ¹H NMR (500 MHz, DMSO-d₆) δ ppm 7.30 (d, 1H), 7.21 (d, 1H), 7.15 (t, 1H), 4.27 (brt, 2H), 4.26 (t, 2H), 4.12 (t, 2H), 3.77 (s, 3H), 3.47 (brt, 2H), 3.26 (t, 2H), 2.89 (t, 2H), 2.82 (brs, 2H), 2.45 (brs, 6H), 2.32 (s, 3H), 2.11 (qn, 2H), 2.04 (qn, 2H), 1.43 (s, 9H); ¹³C NMR (125 MHz, DMSO-d₆) δ ppm 163.1, 155.4, 151.8, 151.4, 151.4, 147.5, 142.4, 136.2, 135, 129.1, 129.1, 119.2, 115.5, 114.8, 82.3, 80.3, 68.3, 56.3, 52.0, 46.4, 46.4, 44.6, 43.1, 30.7, 28.5, 24.2, 23, 19.7, 15.7; HRMS-ESI (m/z): [M+H]⁺ calculated for C₃₄H₄₃ClFN₆O₅S: 701.2683, found 701.2678.

### Step B: methyl 2-[3-(1,3-benzothiazol-2-ylamino)-4-methyl-6,7-dihydro-5H-pyrido[2,3-c]pyridazin-8-yl]-5-[3-[4-[3-[tert-butoxycarbonyl-[2-(dimethylamino)ethyl]amino]prop-1-ynyl]-2-fluoro-phenoxy]propyl]thiazole-4-carboxylate

Using Buchwald General Procedure II starting from the product from Step A and 1,3-benzothiazol-2-amine, the desired product was obtained. LC-MS-ESI (m/z): [M+H]⁺ calculated for C₄₁H₄₈FN₈O₅S₂: 815.3, found 815.4.

### Step C: 2-[3-(1,3-benzothiazol-2-ylamino)-4-methyl-6,7-dihydro-5H-pyrido[2,3-c]pyridazin-8-yl]-5-[3-[4-[3-[2-(dimethylamino)ethylamino]prop-1-ynyl]-2-fluoro-phenoxy]propyl]thiazole-4-carboxylic acid

Using Deprotection and Hydrolysis General Procedure followed by repurification via reverse phase preparative chromatography (C18, 25 mM NH₄HCO₃ in water:MeCN) starting from the product from Step B, the desired product was obtained. HRMS-ESI (m/z): [M+H]⁺ calculated for C₃₅H₃₈FN₈O₃S₂: 701.2487, found 701.2483.

### Preparation of P13: 2-[3-(1,3-Benzothiazol-2-ylamino)-4-methyl-6,7-dihydro-5H pyrido[2,3-c]pyridazin-8-yl]-5-[3-[4-[3-[ethyl(methyl)amino]prop-1-ynyl]-2-fluoro-phenoxy]propyl]thiazole-4-carboxylic acid

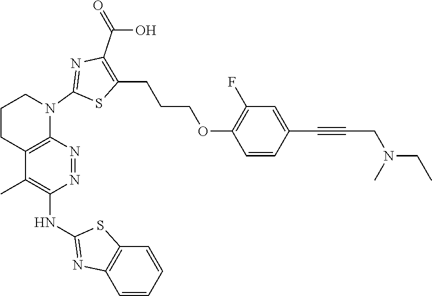 [SMILES: CCN(C)CC#Cc1ccc(OCCCc2sc(N3CCCc4c3nnc(Nc3nc5ccccc5s3)c4C)nc2C(=O)O)c(F)c1]

Using Silver catalyzed propargylic amine preparation General Procedure starting from Preparation 3c, paraformaldehyde as the aldehyde and N-methylethanamine as the appropriate secondary amine, the desired product was obtained. HRMS-ESI (m/z): [M+H]⁺ calculated for C₃₄H₃₅FN₇O₃S₂: 672.2221, found 672.2206.

### Preparation of P14: 2-[3-(1,3-Benzothiazol-2-ylamino)-4-methyl-6,7-dihydro-5H-pyrido[2,3-c]pyridazin-8-yl]-5-[3-[4-[3-(diethylamino)prop-1-ynyl]-2-fluoro-phenoxy]propyl]thiazole-4-carboxylic acid

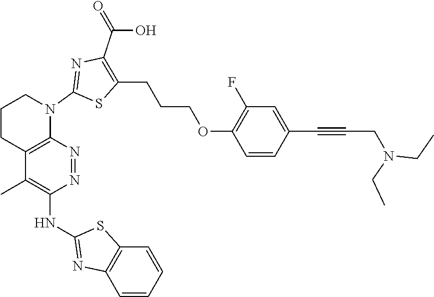 [SMILES: CCN(CC)CC#Cc1ccc(OCCCc2sc(N3CCCc4c3nnc(Nc3nc5ccccc5s3)c4C)nc2C(=O)O)c(F)c1]

Using Silver catalyzed propargylic amine preparation General Procedure starting from Preparation 3c, paraformaldehyde as the aldehyde and diethyl amine as the appropriate secondary amine, the desired product was obtained. HRMS-ESI (m/z): [M+H]⁺ calculated for C₃₅H₃₇FN₇O₃S₂: 686.2377, found 686.2386.

### Preparation of P15: 2-{3-[(1,3-Benzothiazol-2-yl)amino]-4-methyl-5H,6H,7H,8H-pyrido[2,3-c]pyridazin-8-yl}-5-(3-{4-[3-(4,4-difluoropiperidin-1-yl)prop-1-yn-1-yl]-2-fluorophenoxy}propyl)-1,3-thiazole-4-carboxylic acid

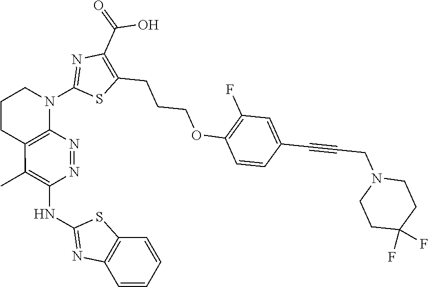 [SMILES: Cc1c(Nc2nc3ccccc3s2)nnc2c1CCCN2c1nc(C(=O)O)c(CCCOc2ccc(C#CCN3CCC(F)(F)CC3)cc2F)s1]

### Step A: methyl 2-[3-(1,3-benzothiazol-2-ylamino)-4-methyl-6,7-dihydro-5H-pyrido[2,3-c]pyridazin-8-yl]-5-[3-[4-[3-(4,4-difluoro-1-piperidyl)prop-1-ynyl]-2-fluoro-phenoxy]propyl]thiazole-4-carboxylate

Using Propargylic amine preparation General Procedure starting from 100 mg of Preparation 3d (0.155 mmol, 1 eq.) as the appropriate propargylic alcohol and 4,4-difluoropiperidine (20 eq.), 120 mg of the desired product (72%) was obtained.

### Step B: 2-{3-[(1,3-benzothiazol-2-yl)amino]-4-methyl-5H,6H,7H,8H-pyrido[2,3-c]pyridazin-8-yl}-5-(3-{4-[3-(4,4-difluoropiperidin-1-yl)prop-1-yn-1-yl]-2-fluorophenoxy}propyl)-1,3-thiazole-4-carboxylic acid

Using Hydrolysis General Procedure starting from the product from Step A as the appropriate methyl ester, the desired product was obtained. HRMS-ESI (m/z): [M+H]⁺ calculated C₃₆H₃₅F3N₇O₃S₂: 734.2189, found 734.2185.

### Preparation of P16: 2-{3-[(1,3-Benzothiazol-2-yl)amino]-4-methyl-6-[2-(methylamino)ethoxy]-5H,6H,7H,8H-pyrido[2,3-c]pyridazin-8-yl}-5-(3-{4-[3-(dimethylamino)prop-1-yn-1-yl]-2-fluorophenoxy}propyl)-1,3-thiazole-4-carboxylic acid

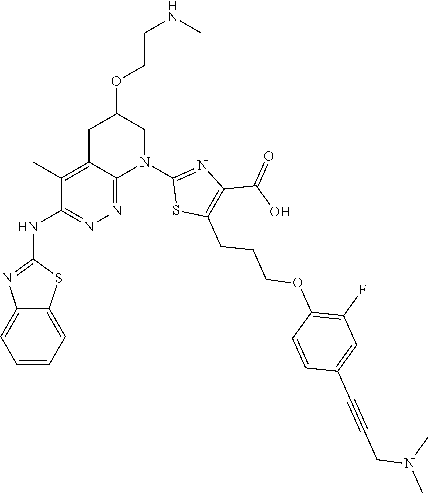 [SMILES: CNCCOC1Cc2c(nnc(Nc3nc4ccccc4s3)c2C)N(c2nc(C(=O)O)c(CCCOc3ccc(C#CCN(C)C)cc3F)s2)C1]

### Step A: 4-methylmorpholin-3-one

A solution of 2-(methylamino) ethanol (5.32 mL, 66.6 mmol, 1 eq) in ethanol (100 mL) and 35% aqueous sodium hydroxide (6.25 mL) was cooled to 15-20° C. and chloroacetyl chloride (13.3 mL, 166 mmol, 2.5 eq) and 35% aqueous sodium hydroxide (22 mL) were added simultaneously with vigorous stirring over 1 h. The mixture was stirred for 20 min, then neutralised with aqueous hydrochloric acid and extracted with dichloromethane (3×100 mL). The combined organic extracts were washed with water, dried (PTFE phase separator) and concentrated in vacuo. Purification by automated flash column chromatography (CombiFlash Rf, 80 g RediSep™ silica cartridge) eluting with a gradient of 0-100% ethyl acetate in iso-heptane afforded the desired product as a colourless oil (4.4 g, 38.2 mmol, 58%). ¹H NMR (400 MHz, DMSO-d₆) δ 4.00 (s, 2H), 3.84-3.78 (m, 2H), 3.36-3.29 (m, 2H), 2.86 (s, 3H).

### Step B: 2-(but-2-yn-1-yl)-4-methylmorpholin-3-one

To a solution of diisopropylamine (6.45 mL, 45.9 mmol, 1.2 eq) in tetrahydrofuran (130 mL), cooled to −78° C., was added n-butyllithium (2.06M in hexanes; 20.4 mL, 42 mmol, 1.1 eq) dropwise. After 1 minute a solution of the product from Step A (4.4 g, 38.2 mmol, 1 eq) in tetrahydrofuran (30 mL) was added dropwise. After 15 minutes a solution of 1-bromo-2-butyne (4.02 mL, 45.9 mmol, 1.2 eq) in tetrahydrofuran (15 mL) was added dropwise and the mixture was stirred at −78° C. for 1 h then allowed to warm to ambient temperature. Saturated aqueous ammonium chloride was added and the mixture was extracted with ethyl acetate (×3), and the combined organic extracts were dried (magnesium sulfate) and concentrated in vacuo. Purification by automated flash column chromatography (CombiFlash Rf, 80 g RediSep™ silica cartridge) eluting with a gradient of 0-100% ethyl acetate in iso-heptane afforded the desired product as a yellow oil (5.15 g, 30.8 mmol, 81%). ¹H NMR (400 MHz, DMSO-d₆) δ 4.09 (dd, J=7.6, 3.5 Hz, 1H), 4.01-3.94 (m, 1H), 3.76 (ddd, J=11.9, 10.0, 3.6 Hz, 1H), 3.52-3.41 (m, 1H), 3.26-3.18 (m, 1H), 2.86 (s, 3H), 2.67-2.58 (m, 1H), 2.57-2.44 (m, 1H), 1.73 (t, J=2.6 Hz, 3H).

### Step C: 2-[2-(methylamino)ethoxy]hex-4-ynoic acid

To a solution of the product from Step B (3.25 g, 19.4 mmol, 1 eq) in methanol (110 mL) was added 1M aqueous lithium hydroxide (60.3 mL, 60.3 mmol, 3.1 eq) and the mixture was heated at reflux overnight. The reaction was concentrated in vacuo to afford the desired product as an orange gum (5.15 g, 27.8 mmol, 100%) that was used directly in the subsequent step without further characterisation.

### Step D: 2-[2-({[(9H-fluoren-9-yl)methoxy]carbonyl}(methyl)amino)ethoxy]hex-4-ynoic acid

To a solution of the product from Step C (5.15 g, 27.8 mmol, 1 eq) in 1,4-dioxane (45 mL) and water (160 mL) was added potassium carbonate (15.4 g, 111 mmol, 4 eq) at 0° C. followed by 9H-fluoren-9-yl-methyl chloroformate (7.19 g, 27.8 mmol, 1 eq) and the mixture was allowed to warm to ambient temperature and stir for 2 h. The reaction was partitioned between water and ethyl acetate, and the aqueous phase was acidified with aqueous hydrochloric acid to pH 2-3 and extracted with ethyl acetate (3×300 mL). The combined organic extracts were washed with brine, dried (magnesium sulfate) and concentrated in vacuo. Purification by automated flash column chromatography (CombiFlash Rf, 120 g RediSep™ silica cartridge) eluting with a gradient of 0-20% methanol in dichloromethane afforded the desired product as a dark yellow gum (7.06 g, 17.3 mmol, 62%). LC/MS (C₂₄H₂₅NO₅) 408 [M+H]⁺; RT 0.74 (LCMS-V-B2). ¹H NMR (400 MHz, DMSO-d₆) δ 7.90 (t, J=6.8 Hz, 2H), 7.65 (dd, J=7.5, 1.1 Hz, 2H), 7.42 (td, J=7.4, 3.0 Hz, 2H), 7.34 (td, J=7.4, 1.3 Hz, 2H), 4.43-4.22 (m, 3H), 3.50-3.42 (m, 1H), 3.39-3.28 (m, 1H), 3.26-3.15 (m, 3H), 2.90-2.82 (m, 3H), 2.51-2.44 (m, 2H), 1.71 (dt, J=13.8, 2.5 Hz, 3H).

### Step E: (9H-fluoren-9-yl)methyl N-{2-[(1-hydroxyhex-4-yn-2-yl)oxy]ethyl}-N-methylcarbamate

A solution of the product from Step D (7.06 g, 17.33 mmol, 1 eq) in tetrahydrofuran (120 mL) was cooled to −10° C., then triethylamine (2.65 mL, 19.1 mmol, 1.1 eq) and isobutyl chloroformate (2.7 mL, 20.8 mmol, 1.2 eq) in THF (40 mL) were added dropwise. The precipitate was removed by filtration and the solution was cooled to −10° C. Sodium borohydride (2.62 g, 69.3 mmol, 4 eq) in water (40 mL) was added dropwise and the mixture was stirred for 1 hr at −10° C. The pH of the solution was adjusted to pH 5 using 1N aqueous hydrochloric acid, and then adjusted to pH 10 using saturated aqueous sodium bicarbonate. The layers were separated and the organic phase was successively washed water (100 mL) and brine (50 mL), dried (magnesium sulfate) and concentrated in vacuo. Purification by automated flash column chromatography (CombiFlash Rf, 80 g RediSep™ silica cartridge) eluting with a gradient of 0-100% ethyl acetate in iso-heptane afforded the desired product as a colourless gum (4.64 g, 11.8 mmol, 68%). LC/MS (C₂₄H₂₇NO₄) 394 [M+H]⁺; RT 0.77 (LCMS-V-B2). ¹H NMR (400 MHz, DMSO-d₆) δ 7.90 (d, J=7.5 Hz, 2H), 7.65 (dt, J=7.4, 0.9 Hz, 2H), 7.43 (t, J=7.4 Hz, 2H), 7.35 (td, J=7.4, 1.2 Hz, 2H), 4.68-4.60 (m, 1H), 4.39 (d, J=6.0 Hz, 1H), 4.34 (d, J=6.7 Hz, 1H), 4.28 (t, J=6.4 Hz, 1H), 3.60-3.51 (m, 1H), 3.46-3.36 (m, 2H), 3.34-3.28 (m, 2H), 3.19 (dd, J=16.6, 5.5 Hz, 2H), 2.84 (d, J=10.8 Hz, 3H), 2.38-2.15 (m, 2H), 1.71 (t, J=2.5 Hz, 3H).

### Step F: (9H-fluoren-9-yl)methyl N-[2-({1-[(tert-butyldiphenylsilyl)oxy]hex-4-yn-2-yl}oxy)ethyl]-N-methylcarbamate

To a cooled solution of the product from Step E (4.64 g, 11.8 mmol, 1 eq) and imidazole (1.56 mL, 23.6 mmol, 2 eq) in dichloromethane (200 mL) was added tert-butyl(chloro)diphenylsilane (6.13 mL, 23.6 mmol, 2 eq) dropwise and the mixture was allowed to warm to ambient temperature and stir overnight. The reaction was quenched with 2M aqueous ammonium chloride and the mixture was extracted with dichloromethane (3×200 mL). The combined organic extracts were washed with brine, dried (PTFE phase separator) and concentrated in vacuo. Purification by automated flash column chromatography (CombiFlash Rf, 120 g RediSep™ silica cartridge) eluting with a gradient of 0-25% ethyl acetate in iso-heptane afforded the desired product as a colourless gum (5.86 g, 9.27 mmol, 79%). LC/MS (C₄₀H₄₅NO₄Si) 632 [M+H]⁺; RT 1.38 (LCMS-V-B2). ¹H NMR (400 MHz, DMSO-d₆) δ 7.87 (dd, J=20.0, 7.5 Hz, 2H), 7.67-7.56 (m, 6H), 7.53-7.39 (m, 7H), 7.39-7.22 (m, 3H), 4.38 (t, J=4.8 Hz, 1H), 4.31 (s, 1H), 4.24 (t, J=5.7 Hz, 1H), 3.73-3.61 (m, 1H), 3.60-3.44 (m, 2H), 3.34 -3.29 (m, 2H), 3.29-3.18 (m, 1H), 3.16-3.06 (m, 1H), 2.81 (d, J=14.1 Hz, 3H), 2.43-2.26 (m, 2H), 1.69 (t, J=2.4 Hz, 3H), 0.98 (s, 9H).

### Step G: (9H-fluoren-9-yl)methyl N-[2-({1-[(tert-butyldiphenylsilyl)oxy]-3-(3,6-dichloro-5-methylpyridazin-4-yl)propan-2-yl}oxy)ethyl]-N-methylcarbamate

A solution of the product from Step F (5.86 g, 9.27 mmol, 1 eq) and 3,6-dichloro-1,2,4,5-tetrazine (5.6 g, 37.1 mmol, 4 eq) in toluene (130 mL) was heated at 150° C. overnight in a sealed flask. The reaction was concentrated in vacuo and purification by automated flash column chromatography (CombiFlash Rf, 120 g RediSep™ silica cartridge) eluting with a gradient of 0-30% ethyl acetate in iso-heptane afforded the desired product as a pink foam (2.99 g, 3.97 mmol, 43%). LC/MS (C₄₂H₄₅Cl₂N₃O₄Si) 754 [M+H]⁺; RT 1.37 (LCMS-V-B2). ¹H NMR (400 MHz, DMSO-d₆) δ 7.90 (d, J=7.7 Hz, 1H), 7.78 (d, J=7.4 Hz, 1H), 7.68-7.59 (m, 5H), 7.57-7.50 (m, 1H), 7.47-7.41 (m, 6H), 7.45-7.37 (m, 1H), 7.36-7.28 (m, 2H), 7.23 (t, J=7.5 Hz, 1H), 4.30 (d, J=5.7 Hz, 1H), 4.27-4.11 (m, 2H), 3.81-3.60 (m, 3H), 3.55-3.45 (m 1H), 3.20-2.98 (m, 4H), 2.89-2.77 (m, 1H), 2.58 (d, J=23.0 Hz, 3H), 2.39 (d, J=13.1 Hz, 3H), 1.01 (s, 9H).

### Step H: 4-{3-[(tert-butyldiphenylsilyl)oxy]-2-[2-(methylamino)ethoxy]propyl}-3,6-dichloro-5-methylpyridazine

A solution of the product from Step G (2.79 g, 3.7 mmol, 1 eq) and diethylamine (0.77 mL, 7.39 mmol, 2 eq) in acetonitrile (60 mL) was stirred at ambient temperature overnight. Water was added and the mixture was extracted with ethyl acetate (3×70 mL). The combined organic extracts were washed with brine (100 mL), dried (magnesium sulfate) and concentrated in vacuo. Purification by automated flash column chromatography (CombiFlash Rf, 40 g RediSep™ silica cartridge) eluting with a gradient of 0-16% methanol in dichloromethane afforded the desired product as an orange/pink gum (1.9 g, 3.57 mmol, 96%). LC/MS (C₂₇H₃₅Cl₂N₃O₂Si) 532 [M+H]⁺; RT 0.84 (LCMS-V-B2). ¹H NMR (400 MHz, DMSO-d₆) δ 7.69-7.62 (m, 4H), 7.54-7.41 (m, 6H), 3.83-3.60 (m, 3H), 3.42-3.36 (m, 1H), 3.16-2.97 (m, 3H), 2.45 (s, 3H), 2.39-2.23 (m, 2H), 2.06 (s, 3H), 1.02 (s, 9H).

### Step I: tert-butyl N-[2-({1-[(tert-butyldiphenylsilyl)oxy]-3-(3,6-dichloro-5-methylpyridazin-4-yl)propan-2-yl}oxy)ethyl]-N-methylcarbamate

To a solution of the product from Step H (1.9 g, 3.57 mmol, 1 eq) in dichloromethane (100 mL) was added di-tert-butyl dicarbonate (1.53 mL, 7.14 mmol, 2 eq) followed by triethylamine (1.99 mL, 14.3 mmol, 4 eq) and the mixture was stirred at ambient temperature for 4 h. The reaction was partitioned between dichloromethane and water, and the aqueous phase was acidified to pH 4 and extracted with dichloromethane (3×80 mL). The combined organic extracts were washed with brine, dried (PTFE phase separator) and concentrated in vacuo. Purification by automated flash column chromatography (CombiFlash Rf, 40 g RediSep™ silica cartridge) eluting with a gradient of 0-25% ethyl acetate in iso-heptane afforded the desired product as a colourless gum (1.83 g, 2.9 mmol, 81%). LC/MS (C₃₂H₄₃Cl₂N₃O₄Si) 532 [M−Boc+H]⁺; RT 1.33 (LCMS-V-B2). ¹H NMR (400 MHz, DMSO-d₆) δ 7.69-7.62 (m, 4H), 7.54-7.41 (m, 6H), 3.76 (qd, J=10.7, 4.7 Hz, 2H), 3.66 (d, J=5.5 Hz, 1H), 3.44 (q, J=7.9, 6.3 Hz, 1H), 3.20-3.10 (m, 3H), 3.04 (dd, J=14.0, 4.1 Hz, 2H), 2.58 (s, 3H), 2.44 (s, 3H), 1.31 (d, J=22.6 Hz, 9H), 1.02 (s, 9H).

### Step J: tert-butyl N-(2-{[1-(3,6-dichloro-5-methylpyridazin-4-yl)-3-hydroxypropan-2-yl]oxy}ethyl)-N-methylcarbamate

A solution of the product from Step I (1.83 g, 2.9 mmol, 1 eq) in tetrahydrofuran (75 mL) was cooled to 0° C. before the addition of tetrabutylammonium fluoride (1M in tetrahydrofuran; 2.9 mL, 2.9 mmol, 1 eq) and stirring at 0° C. for 30 min, then at ambient temperature for 1 h. The reaction was partitioned between dichloromethane and water, and the aqueous phase was extracted with dichloromethane (×2). The combined organic extracts were washed with brine, dried (PTFE phase separator) and concentrated in vacuo. Purification by automated flash column chromatography (CombiFlash Rf, 24 g RediSep™ silica cartridge) eluting with a gradient of 0-100% ethyl acetate in iso-heptane afforded the desired product as a pale orange gum (0.73 g, 1.86 mmol, 64%). ¹H NMR (400 MHz, DMSO-d₆) δ 4.93 (t, J=5.5 Hz, 1H), 3.62-3.44 (m, 4H), 3.23 (dt, J=9.6, 6.0 Hz, 1H), 3.11 (d, J=23.9 Hz, 2H), 3.02 (dd, J=6.5, 2.0 Hz, 2H), 2.60 (d, J=8.1 Hz, 3H), 2.45 (s, 3H), 1.35 (d, J=13.0 Hz, 9H).

### Step K: methyl 2-{[(tert-butoxy)carbonyl][2-(2-{[(tert-butoxy)carbonyl](methyl)amino}ethoxy)-3-(3,6-dichloro-5-methylpyridazin-4-yl)propyl]amino}-5-(3-{4-[3-(dimethylamino)prop-1-yn-1-yl]-2-fluorophenoxy}propyl)-1,3-thiazole-4-carboxylate

To a solution of the product from Step J (125 mg, 0.32 mmol, 1 eq) in toluene (20 mL) was added the product from Preparation 1c (171 mg, 0.35 mmol, 1.1 eq), di-tert-butyl azodicarboxylate (146 mg, 0.63 mmol, 2 eq) and triphenylphosphine (166 mg, 0.63 mmol, 2 eq) and the mixture was stirred at 50° C. for 1 h. The reaction was partitioned between dichloromethane and water, and the aqueous phase was extracted with dichloromethane (×2), and the combined organic extracts were washed with brine, dried (magnesium sulfate) and concentrated in vacuo. Purification by automated flash column chromatography (CombiFlash Rf, 12 g RediSep™ silica cartridge) eluting with a gradient of 0-100% ethyl acetate in iso-heptane afforded the desired product as a pale yellow gum (282 mg, 0.32 mmol, 102%). LC/MS (C₄₀H₅₃Cl₂FN₆O₈S) 867 [M+H]⁺; RT 0.97 (LCMS-V-B2). ¹H NMR (400 MHz, DMSO-d₆) δ 7.30 (dd, 1H), 7.23-7.17 (m, 1H), 7.12 (t, 1H), 4.29 (dd, J=13.9, 5.7 Hz, 1H), 4.10 (t, J=6.0 Hz, 2H), 3.96-3.87 (m, 1H), 3.74 (s, 3H), 3.61-3.48 (m, 1H), 3.42 (s, 3H), 3.32 (s, 2H), 3.25 (dt, J=7.1, 3.9 Hz, 3H), 3.16-2.99 (m, 2H), 2.97-2.89 (m, 1H), 2.58 (d, J=11.6 Hz, 2H), 2.45 (s, 3H), 2.23 (s, 6H), 2.10 (t, J=6.9 Hz, 2H), 1.52 (s, 9H), 1.31 (d, J=39.6 Hz, 9H).

### Step L: methyl 2-{[2-(2-{[(tert-butoxy)carbonyl](methyl)amino}ethoxy)-3-(3,6-dichloro-5-methylpyridazin-4-yl)propyl]amino}-5-(3-{4-[3-(dimethylamino)prop-1-yn-1-yl]-2-fluorophenoxy}propyl)-1,3-thiazole-4-carboxylate

A solution of the product from Step K (275 mg, 0.32 mmol, 1 eq) in 1,1,1,3,3,3-hexafluoro-2-propanol (2.5 mL, 23.7 mmol, 74.7 eq) was heated at 100° C. for 60 min under microwave irradiation. The reaction was concentrated in vacuo and purification by automated flash column chromatography (CombiFlash Rf, 12 g RediSep™ silica cartridge) eluting with a gradient of 0-7% methanol in dichloromethane afforded the desired product as a white solid (154 mg, 0.2 mmol, 63%). LC/MS (C₃₅H₄₅Cl₂FN₆O₆S) 767 [M+H]⁺; RT 0.70 (LCMS-V-B2). ¹H NMR (400 MHz, DMSO-d₆) δ 7.83 (br s, 1H), 7.30 (dd, J=11.9, 2.0 Hz, 1H), 7.24-7.17 (m, 1H), 7.12 (t, J=8.7 Hz, 1H), 4.08 (t, J=6.1 Hz, 2H), 3.82 (dt, J=9.0, 4.5 Hz, 1H), 3.70 (s, 3H), 3.60-3.49 (m, 1H), 3.46-3.39 (m, 4H), 3.33 (s, 2H), 3.29-3.18 (m, 1H), 3.14 (t, 2H), 3.10-3.02 (m, 2H), 2.98 (dd, J=13.9, 3.8 Hz, 1H), 2.64-2.53 (m, 2H), 2.44 (s, 3H), 2.23 (s, 6H), 2.07-1.95 (m, 2H), 1.32 (d, J=30.8 Hz, 9H).

### Step M: methyl 2-[6-(2-{[(tert-butoxy)carbonyl](methyl)amino}ethoxy)-3-chloro-4-methyl-5H,6H,7H,8H-pyrido[2,3-c]pyridazin-8-yl]-5-(3-{4-[3-(dimethylamino)prop-1-yn-1-yl]-2-fluorophenoxy}propyl)-1,3-thiazole-4-carboxylate

To a solution of the product from Step L (154 mg, 0.2 mmol, 1 eq) in 1,4-dioxane (14 mL) was added cesium carbonate (131 mg, 0.4 mmol, 2 eq), N,N-diisopropylethylamine (0.07 mL, 0.4 mmol, 2 eq) and bis(di-tert-butyl(4-dimethylaminophenyl)phosphine) dichloropalladium(II)(14.2 mg, 0.02 mmol, 0.1 eq) and the mixture was heated at 80° C. for 45 min. The reaction was partitioned between dichloromethane and water, and the aqueous phase was extracted with dichloromethane (×2). The combined organic extracts were washed with brine, dried (magnesium sulfate) and concentrated in vacuo. Purification by automated flash column chromatography (CombiFlash Rf, 12 g RediSep™ silica cartridge) eluting with a gradient of 0-8% methanol in dichloromethane afforded the desired product as a cream solid (136 mg, 0.19 mmol, 93%). LC/MS (C₃₅H₄₄ClFN₆O₆S) 731 [M+H]⁺; RT 0.75 (LCMS-V-B2). ¹H NMR (400 MHz, DMSO-d₆) δ 7.31 (dt, J=12.0, 1.9 Hz, 1H), 7.25-7.19 (m, 1H), 7.14 (t, 1H), 4.86 (dd, 1H), 4.25 (s, 1H), 4.13 (t, J=6.2 Hz, 2H), 3.93 (d, J=13.5 Hz, 1H), 3.78 (s, 3H), 3.56 (t, J=5.6 Hz, 2H), 3.42 (s, 3H), 3.32 (s, 2H), 3.30-3.23 (m, 2H), 3.21-3.09 (m, 2H), 3.08-3.00 (m, 1H), 2.58-2.52 (m, 1H), 2.34 (s, 3H), 2.23 (s, 6H), 2.12 (p, J=6.7 Hz, 2H), 1.27 (d, J=28.5 Hz, 9H).

### Step N: methyl 2-{3-[(1,3-benzothiazol-2-yl)amino]-6-(2-{[(tert-butoxy)carbonyl](methyl)amino}ethoxy)-4-methyl-5H,6H,7H,8H-pyrido[2,3-c]pyridazin-8-yl}-5-(3-{4-[3-(dimethylamino)prop-1-yn-1-yl]-2-fluorophenoxy}propyl)-1,3-thiazole-4-carboxylate

To a solution of the product from Step M (136 mg, 0.19 mmol, 1 eq) in cyclohexanol (4.5 mL) was added 2-aminobenzothiazole (55.7 mg, 0.37 mmol, 2 eq) and N,N-diisopropylethylamine (0.1 mL, 0.56 mmol, 3 eq) and the mixture was sparged with nitrogen (10 min). Xantphos (21.5 mg, 0.04 mmol, 0.2 eq) and tris(dibenzylideneacetone)dipalladium(0)(17 mg, 0.02 mmol, 0.1 eq) were added and the mixture was heated at 140° C. for 1 h under microwave irradiation. The reaction was partitioned between dichloromethane and water, and the aqueous phase was extracted with dichloromethane (3×40 mL). The combined organic extracts were washed with brine, dried (PTFE phase separator) and concentrated in vacuo. Purification by reverse phase automated flash chromatography (CombiFlash Rf, C18 15.5g Gold RediSep column) eluting with a gradient of 5-95% acetonitrile in water afforded the desired product as a yellow solid (70.8 mg, 0.08 mmol, 45%). LC/MS (C₄₂H₄₉FN₈O₆S₂) 845 [M+H]⁺; RT 0.86 (LCMS-V-B2). ¹H NMR (400 MHz, DMSO-d₆) δ 11.52 (br s, 1H), 7.88 (d, J=7.8 Hz, 1H), 7.49 (d, J=8.1 Hz, 1H), 7.37 (ddd, J=8.2, 7.3, 1.3 Hz, 1H), 7.31 (dd, J=11.9, 1.9 Hz, 1H), 7.24-7.12 (m, 3H), 4.80 (dd, 1H), 4.22 (s, 1H), 4.15 (t, J=6.2 Hz, 2H), 3.94 (d, J=13.4 Hz, 1H), 3.78 (s, 3H), 3.56 (t, J=5.7 Hz, 2H), 3.44-3.37 (m, 1H), 3.31 (s, 2H), 3.28 (d, 1H), 3.24-3.14 (m, 2H), 3.12-2.97 (m, 2H), 2.58 (d, J=12.3 Hz, 3H), 2.33 (s, 3H), 2.19 (s, 6H), 2.14 (q, J=7.0 Hz, 2H), 1.27 (d, 9H).

### Step O: methyl 2-{3-[(1,3-benzothiazol-2-yl)amino]-4-methyl-6-[2-(methylamino)ethoxy]-5H,6H,7H,8H-pyrido[2,3-c]pyridazin-8-yl}-5-(3-{4-[3-(dimethylamino)prop-1-yn-1-yl]-2-fluorophenoxy}propyl)-1,3-thiazole-4-carboxylate

To a solution of the product from Step N (70.8 mg, 0.08 mmol, 1 eq) in dichloromethane (5 mL) was added trifluoroacetic acid (1 mL) slowly and the mixture was stirred at ambient temperature for 1 h. The reaction was partitioned between dichloromethane and saturated aqueous sodium bicarbonate and the aqueous phase was extracted with dichloromethane (3×30 mL). The combined organic extracts were washed with brine, dried (PTFE phase separator) and concentrated in vacuo to afford the desired product as a bright yellow solid (59.8 mg, 0.08 mmol, 96%). LC/MS (C₃₇H₄₁FN₈O₄S₂) 745 [M+H]⁺; RT 1.07 (LCMS-V-B1). ¹H NMR (400 MHz, DMSO-d₆) δ 7.88 (dd, J=7.8, 1.2 Hz, 1H), 7.49 (d, J=8.1 Hz, 1H), 7.37 (ddd, J=8.2, 7.2, 1.3 Hz, 1H), 7.32 (dd, J=11.9, 1.9 Hz, 1H), 7.24-7.12 (m, 3H), 4.79-4.69 (m, 1H), 4.26-4.19 (m, 1H), 4.15 (t, J=6.2 Hz, 2H), 4.03 (dd, J=13.5, 2.4 Hz, 1H), 3.78 (s, 3H), 3.60 (t, J=5.5 Hz, 2H), 3.39 (s, 2H), 3.32-3.27 (m, 2H), 3.15 (d, J=14.6 Hz, 1H), 3.08-2.99 (m, 1H), 2.70 (t, J=5.5 Hz, 2H), 2.38 (s, 3H), 2.29 (s, 3H), 2.22 (s, 6H), 2.17-2.08 (m, 2H).

### Step P: 2-{3-[(1,3-benzothiazol-2-yl)amino]-4-methyl-6-[2-(methylamino)ethoxy]-5H,6H,7H,8H-pyrido[2,3-c]pyridazin-8-yl}-5-(3-{4-[3-(dimethylamino)prop-1-yn-1-yl]-2-fluorophenoxy}propyl)-1,3-thiazole-4-carboxylic acid

To a solution of the product from Step O (59.8 mg, 0.08 mmol, 1 eq) in 1,4-dioxane (2 mL) was added 1M aqueous lithium hydroxide (0.24 mL, 0.24 mmol, 3 eq) and the mixture was heated at 50° C. for 2 h. The solid was collected by filtration and dried under vacuum to afford the desired product as a bright yellow solid (43 mg, 0.06 mmol, 73%), as a lithium salt. HRMS-ESI (m/z) [M+H]⁺ calculated for C₃₆H₄₀FN₈O₄S₂: 731.2598, found 731.2623.

### Preparation of P17: 2-[3-(1,3-Benzothiazol-2-ylamino)-4-methyl-6,7-dihydro-5H-pyrido[2,3-c]pyridazin-8-yl]-5-[3-[2-fluoro-4-(3-piperazin-1-ylprop-1-ynyl)phenoxy]propyl]thiazole-4-carboxylic acid

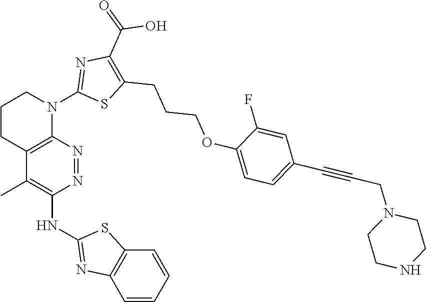 [SMILES: Cc1c(Nc2nc3ccccc3s2)nnc2c1CCCN2c1nc(C(=O)O)c(CCCOc2ccc(C#CCN3CCNCC3)cc2F)s1]

### Step A: 2-[3-(1,3-benzothiazol-2-ylamino)-4-methyl-6,7-dihydro-5H-pyrido[2,3-c]pyridazin-8-yl]-5-[3-[4-[3-(4-tert-butoxycarbonylpiperazin-1-yl)prop-1-ynyl]-2-fluoro-phenoxy]propyl]thiazole-4-carboxylic acid

Using Silver catalyzed propargylic amine preparation General Procedure starting from Preparation 3c, paraformaldehyde as the aldehyde and tert-butyl piperazine-1-carboxylate as the appropriate secondary amine, the desired product was obtained.

### Step B: 2-[3-(1,3-benzothiazol-2-ylamino)-4-methyl-6,7-dihydro-5H-pyrido[2,3-c]pyridazin-8-yl]-5-[3-[2-fluoro-4-(3-piperazin-1-ylprop-1-ynyl)phenoxy]propyl]thiazole-4-carboxylic acid

The mixture of the product from Step A (207 mg, 0.25 mmol) and HF×Pyr (2.5 mmol, 10 eq.) in acetonitrile (4.3 mL) was stirred at 60° C. for 2.5 h. The product was purified via flash chromatography on 24 g silica gel column using DCM and MeOH (NH₃) as eluents to give 143 mg (79%) of the desired product. HRMS-ESI (m/z): [M+H]⁺ calculated for C₃₅H₃₆FN₈O₃S₂: 699.2330, found 699.2322.

### Preparation of P18: 2-[3-(1,3-Benzothiazol-2-ylamino)-4-methyl-6,7-dihydro-5H-pyrido[2,3-c]pyridazin-8-yl]-5-[3-[2-fluoro-4-[3-(1-piperidyl)prop-1-ynyl]phenoxy]propyl]thiazole-4-carboxylic acid

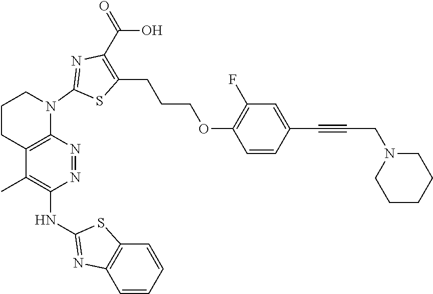 [SMILES: Cc1c(Nc2nc3ccccc3s2)nnc2c1CCCN2c1nc(C(=O)O)c(CCCOc2ccc(C#CCN3CCCCC3)cc2F)s1]

### Step A: methyl 2-[3-(1,3-benzothiazol-2-ylamino)-4-methyl-6,7-dihydro-5H-pyrido[2,3-c]pyridazin-8-yl]-5-[3-[2-fluoro-4-[3-(1-piperidyl)prop-1-ynyl]phenoxy]propyl]thiazole-4-carboxylate

Using Propargylic amine preparation General Procedure starting from 100 mg of Preparation 3d (0.155 mmol, 1 eq.) as the appropriate propargylic alcohol and piperidine (264.2 mg, 20 eq.), 55 mg of the desired product (50%) was obtained.

### Step B: 2-[3-(1,3-benzothiazol-2-ylamino)-4-methyl-6,7-dihydro-5H-pyrido[2,3-c]pyridazin-8-yl]-5-[3-[2-fluoro-4-[3-(1-piperidyl)prop-1-ynyl]phenoxy]propyl]thiazole-4-carboxylic acid

Using Hydrolysis General Procedure starting from the product of Step A as the appropriate methyl ester, the desired product was obtained. HRMS-ESI (m/z): [M+H]⁺ calculated for C₃₆H₃₇FN₇O₃S₂: 698.2377, found 698.2373.

### Preparation of P19: 6-{3-[(1,3-benzothiazol-2-yl)amino]-4-methyl-6,7-dihydropyrido[2,3-c]pyridazin-8 (5H)-yl}-3-(1-{[3-(2-{[(3S)-3,4-dihydroxybutyl]amino}ethoxy)-5,7-dimethyladamantan-1-yl]methyl}-5-methyl-1H-pyrazol-4-yl)pyridine-2-carboxylic acid

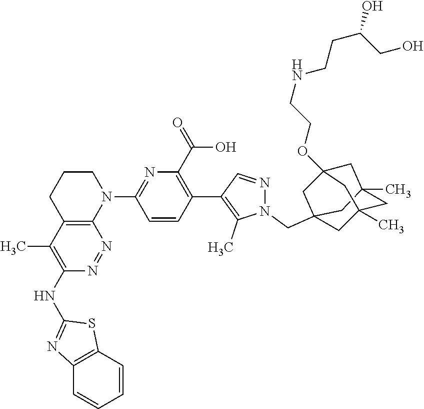 [SMILES: Cc1c(Nc2nc3ccccc3s2)nnc2c1CCCN2c1ccc(-c2cnn(CC34CC5(C)CC(C)(C3)CC(OCCNCC[C@H](O)CO)(C5)C4)c2C)c(C(=O)O)n1]

Using the Amine substitution and Hydrolysis General procedure I starting from Preparation 12 and 2-[(4S)-2,2-dimethyl-1,3-dioxolan-4-yl]ethanamine as the appropriate amine, a compound with a dihydroxy protected amine was obtained. Hydrolysis with a 10% HCl solution (rt, 1 h) and purification by preparative HPLC (using acetonitrile and 5 mM aqueous NH₄HCO₃ solution as eluents) afforded the desired product. HRMS-ESI (m/z): [M+H]⁺ calculated for C₄₄H₅₅N₉O₅: 822.4125, found: 822.4120.

### Preparation of P20: 6-[3-(1,3-benzothiazol-2-ylamino)-4-methyl-6,7-dihydro-5H-pyrido[2,3-c]pyridazin-8-yl]-3-[1-[[3,5-dimethyl-7-[2-(4-methylpiperazin-1-yl)ethoxy]-1-adamantyl]methyl]-5-methyl-pyrazol-4-yl]pyridine-2-carboxylic acid

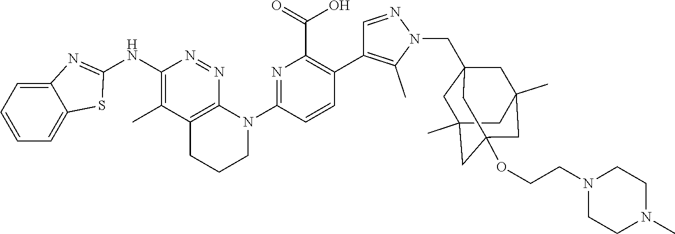 [SMILES: Cc1c(Nc2nc3ccccc3s2)nnc2c1CCCN2c1ccc(-c2cnn(CC34CC5(C)CC(C)(C3)CC(OCCN3CCN(C)CC3)(C5)C4)c2C)c(C(=O)O)n1]

Using the Amine substitution and Hydrolysis General procedure I starting from Preparation 12 and 1-methylpiperazine as the appropriate amine, the desired product was obtained. HRMS-ESI (m/z): [M+2H]2+ calculated for C₄₅H₅₈N₁₀O₃S: 409.2207, found: 409.2208.

### Preparation of P21: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]pyridine-2-carboxylic acid

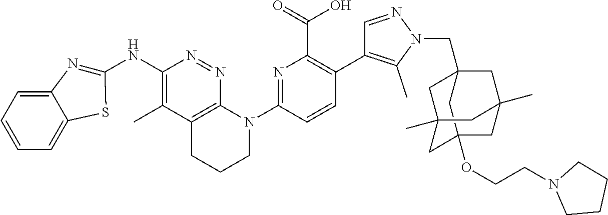 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)O)n1]

Using the Amine substitution and Hydrolysis General procedure I starting from Preparation 12 and pyrrolidine as the appropriate amine, the desired product was obtained. HRMS-ESI (m/z): [M+H]⁺ calculated for C₄₄H₅₄N₉O₃S: 788,4070, found: 788.4068.

### Preparation of P22: 6-[3-(1,3-benzothiazol-2-ylamino)-4-methyl-6,7-dihydro-5H-pyrido[2,3-c]pyridazin-8-yl]-3-[1-[[3-[2-(4-hydroxybutylamino)ethoxy]-5,7-dimethyl-1-adamantyl]methyl]-5-methyl-pyrazol-4-yl]pyridine-2-carboxylic acid

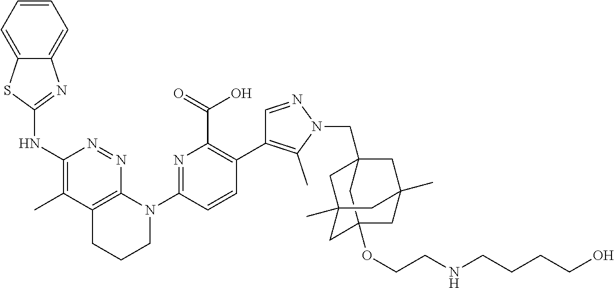 [SMILES: Cc1c(Nc2nc3ccccc3s2)nnc2c1CCCN2c1ccc(-c2cnn(CC34CC5(C)CC(C)(C3)CC(OCCNCCCCO)(C5)C4)c2C)c(C(=O)O)n1]

Using the Amine substitution and Hydrolysis General procedure I starting from Preparation 12 and 4-aminobutan-1-ol as the appropriate amine, the desired product was obtained. HRMS-ESI (m/z): [M+H]⁺ calculated for C₄₄H₅₆N₉O₄S: 806.4176, found: 806.4174.

### Preparation of P23: 6-[3-(1,3-benzothiazol-2-ylamino)-4-methyl-6,7-dihydro-5H-pyrido[2,3-c]pyridazin-8-yl]-3-[1-[[3-[2-[[3-hydroxy-2-(hydroxymethyl)propyl]amino]ethoxy]-5,7-dimethyl-1-adamantyl]methyl]-5-methyl-pyrazol-4-yl]pyridine-2-carboxylic acid

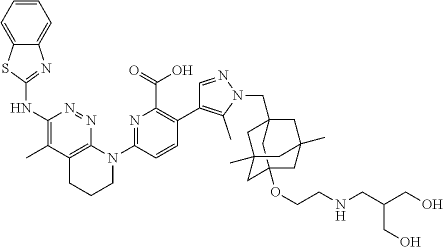 [SMILES: Cc1c(Nc2nc3ccccc3s2)nnc2c1CCCN2c1ccc(-c2cnn(CC34CC5(C)CC(C)(C3)CC(OCCNCC(CO)CO)(C5)C4)c2C)c(C(=O)O)n1]

Using the Amine substitution and Hydrolysis General procedure I starting from Preparation 12 and (2,2-dimethyl-1,3-dioxan-5-yl) methanamine as the appropriate amine a compound with a dihydroxy protected amine was obtained. Hydrolysis with a 10% HCl solution (rt, 1 h) and purification by preparative HPLC (using acetonitrile and 5 mM aqueous NH₄HCO₃ solution as eluents) afforded the desired product. HRMS-ESI (m/z): [M+H]⁺ calculated for C₄₄H₅₆N₉O₅S: 822,4125, found: 822.4099.

### Preparation of P24: 6-[3-(1,3-benzothiazol-2-ylamino)-4-methyl-6,7-dihydro-5H-pyrido[2,3-c]pyridazin-8-yl]-3-[1-[[3-[2-[[2-hydroxy-1-(hydroxymethyl)ethyl]amino]ethoxy]-5,7-dimethyl-1-adamantyl]methyl]-5-methyl-pyrazol-4-yl]pyridine-2-carboxylic acid

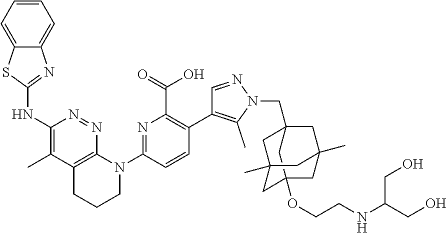 [SMILES: Cc1c(Nc2nc3ccccc3s2)nnc2c1CCCN2c1ccc(-c2cnn(CC34CC5(C)CC(C)(C3)CC(OCCNC(CO)CO)(C5)C4)c2C)c(C(=O)O)n1]

Using the Amine substitution and Hydrolysis General procedure I starting from Preparation 12 and 2-aminopropane-1,3-diol as the appropriate amine, the desired product was obtained. HRMS-ESI (m/z): [M+H]⁺ calculated for C₄₃H₅₄N₉O₅S: 808.3969, found: 808.3965.

### Preparation of P25: 6-[3-(1,3-benzothiazol-2-ylamino)-4-methyl-6,7-dihydro-5H-pyrido[2,3-c]pyridazin-8-yl]-3-[1-[[3-[2-(3-hydroxypropylamino)ethoxy]-5,7-dimethyl-1-adamantyl]methyl]-5-methyl-pyrazol-4-yl]pyridine-2-carboxylic acid

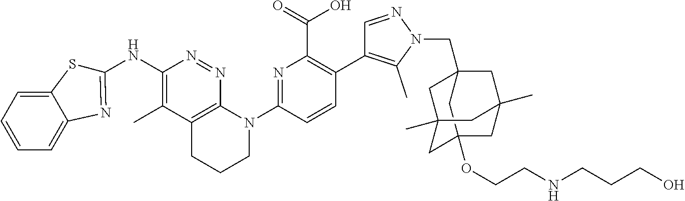 [SMILES: Cc1c(Nc2nc3ccccc3s2)nnc2c1CCCN2c1ccc(-c2cnn(CC34CC5(C)CC(C)(C3)CC(OCCNCCCO)(C5)C4)c2C)c(C(=O)O)n1]

Using the Amine substitution and Hydrolysis General procedure I starting from Preparation 12 and 3-aminopropan-1-ol as the appropriate amine, the desired product was obtained. HRMS-ESI (m/z): [M+H]⁺ calculated for C₄₃H₅₄N₉O₄S: 792.4019, found: 792.4012.

### Preparation of P26: 6-[[6-(1,3-benzothiazol-2-ylamino)-5-methyl-pyridazin-3-yl]-methyl-amino]-3-[1-[[3-[2-(3-hydroxypropylamino)ethoxy]-5,7-dimethyl-1-adamantyl]methyl]-5-methyl-pyrazol-4-yl]pyridine-2-carboxylic acid

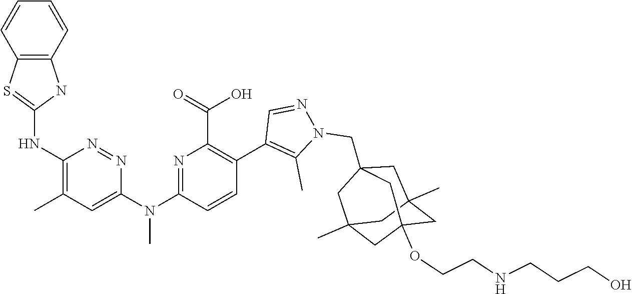

Using the Amine Substitution and Hydrolysis General procedure II starting from Preparation 14_01 and 3-aminopropane-1-ol as the appropriate amine, the desired product was obtained. HRMS-ESI (m/z): [M+H]⁺ calculated for C₄₁H₅₂N₉O₄S: 766.3863, found: 766.3860.

### Preparation of P27: 6-{3-[(1,3-benzothiazol-2-yl)amino]-4-methyl-6,7-dihydropyrido[2,3-c]pyridazin-8 (5H)-yl}-3-[1-({3-[2-(dimethylamino)ethoxy]-5,7-dimethyladamantan-1-yl}methyl)-5-methyl-1H-pyrazol-4-yl]pyridine-2-carboxylic acid

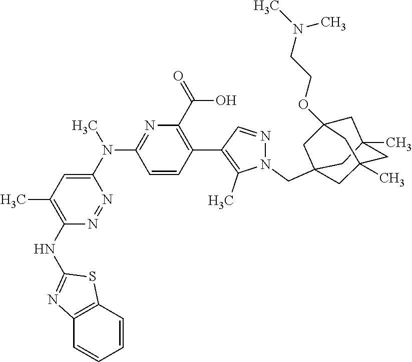

Using the Amine substitution and Hydrolysis General procedure I starting from Preparation 12 and dimethylamine as the appropriate amine, the desired product was obtained. HRMS-ESI (m/z): [M+H]⁺ calculated for C₄₂H₅₂N₉O₃S: 762.3914, found: 762.3912.

### Preparation of P28: 2-[3-(1,3-benzothiazol-2-ylamino)-4-methyl-6,7-dihydro-5H-pyrido[2,3-c]pyridazin-8-yl]-5-[3-[4-[3-(dimethylamino)prop-1-ynyl]phenoxy]propyl]thiazole-4-carboxylic acid

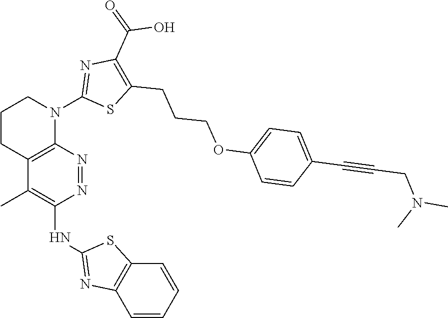 [SMILES: Cc1c(Nc2nc3ccccc3s2)nnc2c1CCCN2c1nc(C(=O)O)c(CCCOc2ccc(C#CCN(C)C)cc2)s1]

### Step A: 4-[3-(dimethylamino)prop-1-ynyl]phenol

Using Sonogashira General Procedure starting from 10.0 g of 4-iodophenol (45.45 mmol) and 4.91 g (1.3 eq) of N,N-dimethylprop-2-yn-1-amine, 3.29 g (41%) of the desired product was obtained. ¹H NMR (400 MHz, DMSO-d₆) δ ppm 9.83 (brs, 1H), 7.25 (d, 2H), 6.74 (d, 2H), 3.44 (s, 2H), 2.26 (s, 6H); LC/MS (C₁₁H₁₄NO) 176[M+H]⁺.

### Step B: methyl 2-(tert-butoxycarbonylamino)-5-[3-[tert-butyl(diphenyl)silyl]oxypropyl]thiazole-4-carboxylate

To the product of Preparation 1a, Step C (77.0 g, 243.7 mmol), imidazole (33.14 g, 2 eq) and DMAP (1.49 g, 0.05 eq) in DMF (973 mL) was added dropwise tert-butyl(chloro)diphenylsilane (93.5 mL, 1.5 eq) and the reaction mixture was stirred at rt for 16 h. After removal of the volatiles, purification by column chromatography (silica gel, using heptane and EtOAc as eluents) afforded 13.56 g (99%) of the desired product. ¹H NMR (500 MHz, DMSO-d₆) δ ppm 11.63 (s, 1H), 7.60 (d, 4H), 7.45 (t, 2H), 7.42 (t, 4H), 3.74 (s, 3H), 3.67 (t, 2H), 3.20 (t, 2H), 1.87 (qn, 2H), 1.47 (s, 9H), 0.99 (s, 9H); ¹³C NMR (125 MHz, DMSO-d₆) δ ppm 162.8, 156.0, 142.6, 135.6, 135.5, 133.5, 130.3, 128.3, 81.8, 62.9, 51.9, 34.0, 28.3, 27.1, 23.2, 19.2; HRMS-ESI (m/z): [M+H]⁺ calculated for C₂₉H₃₉N₂O₅SSi: 555.2349, found: 555.2336.

### Step C: methyl 2-[tert-butoxycarbonyl-[3-(3,6-dichloro-5-methyl-pyridazin-4-yl)propyl]amino]-5-[3-[tert-butyl(diphenyl)silyl]oxypropyl]thiazole-4-carboxylate

Using Alkylation General Procedure starting from 34.95 g (63 mmol) of the product from Step B and 25.0 g (1.2 eq) of 3,6-dichloro-4-(3-iodopropyl)-5-methyl-pyridazine as the appropriate iodine compound, 51.0 g (quantitative yield) of the desired product was obtained. ¹H NMR (500 MHz, DMSO-d₆) δ ppm 7.63-7.37 (m, 10H), 4.09 (t, 2H), 3.75 (s, 3H), 3.67 (t, 2H), 3.20 (t, 2H), 2.82 (m, 2H), 2.40 (s, 3H), 1.87 (m, 2H), 1.87 (m, 2H), 1.50 (s, 9H), 0.97 (s, 9H); ¹³C NMR (125 MHz, DMSO-d₆) δ ppm 62.9, 52.0, 46.1, 33.9, 28.1, 27.5, 27.1, 25.9, 23.8, 16.4; HRMS-ESI (m/z): [M+H]⁺ calculated for C₃₇H₄₇C₁₂N₄O₅SSi: 757.2413, found: 757.2395.

### Step D: methyl 5-[3-[tert-butyl(diphenyl)silyl]oxypropyl]-2-[3-(3,6-dichloro-5-methyl-pyridazin-4-yl)propylamino]thiazole-4-carboxylate

Using Deprotection with HFIP General Procedure starting from 51.70 g of the product from Step C (68 mmol), 36.32 g (81%) of the desired product was obtained. ¹H NMR (500 MHz, DMSO-d₆) δ ppm 7.71 (t, 1H), 7.63-7.37 (m, 10H), 3.69 (s, 3H), 3.67 (t, 2H), 3.30 (m, 2H), 3.10 (t, 2H), 2.85 (m, 2H), 2.83 (s, 3H), 1.79 (m, 2H), 1.78 (m, 2H), 0.98 (s, 9H); ¹³C NMR (125 MHz, DMSO-d₆) δ ppm 62.9, 51.7, 44.1, 34.2, 28.0, 27.1, 27.0, 23.4, 16.4; HRMS-ESI (m/z): [M+H]⁺ calculated for C₃₂H₃₉Cl₂N₄O₃SSi: 657.1889, found: 657.1875.

### Step E: methyl 5-[3-[tert-butyl(diphenyl)silyl]oxypropyl]-2-(3-chloro-4-methyl-6,7-dihydro-5H-pyrido[2,3-c]pyridazin-8-yl)thiazole-4-carboxylate

The mixture of 36.0 g (54.7 mmol) of the product from Step D and 35.7 g (2 eq) of Cs₂CO₃ in 1,4-dioxane (383 mL) was stirred at 90° C. for 18 h. After dilution with water, the precipitated solid was filtered off, washed with diethylether, and dried to give 34.0 g (99%) of the desired product. ¹H NMR (500 MHz, DMSO-d₆) δ ppm 7.61 (d, 4H), 7.43 (t, 2H), 7.42 (t, 4H), 4.26 (t, 2H), 3.77 (s, 3H), 3.70 (t, 2H), 3.23 (t, 2H), 2.90 (t, 2H), 2.33 (s, 3H), 2.04 (qn, 2H), 1.90 (qn, 2H), 1.00 (s, 9H); ¹³C NMR (125 MHz, DMSO-d₆) δ ppm 163.1, 155.3, 151.8, 151.4, 143.2, 136.2, 135.5, 134.7, 133.6, 130.3, 129.0, 128.3, 63.1, 51.9, 46.3, 34.1, 27.1, 24.2, 23.1, 19.8, 19.2, 15.7; HRMS-ESI (m/z): [M+H]⁺ calculated for C₃₂H₃₈ClN₄O₃SSi: 621.2122, found: 621.2097.

### Step F: methyl 2-(3-chloro-4-methyl-6,7-dihydro-5H-pyrido[2,3-c]pyridazin-8-yl)-5-(3-hydroxypropyl)thiazole-4-carboxylate

The mixture of 23.36 g (37.6 mmol) of the product from Step E and 45 mL (1.2 eq.) of 1 M TBAF solution in THF (5 mL/mmol) was stirred at rt for 2 h. After the removal of the volatiles, purificationby column chromatography (silica gel, using EtOAc and MeOH/NH₃ as eluents) afforded 12.88 g (89%) of the desired product. ¹H NMR (500 MHz, DMSO-d₆) δ ppm 4.54 (br., 1H), 4.25 (m, 2H), 3.80 (s, 3H), 3.45 (t, 2H), 3.11 (m, 2H), 2.88 (t, 2H), 2.31 (s, 3H), 2.04 (m, 2H), 1.77 (m, 2H); ¹³C NMR (125 MHz, DMSO-d₆) δ ppm 163.1, 155.2, 151.2, 143.8, 136.1, 134.5, 129.0, 60.5, 52.0, 46.3, 34.6, 24.2, 23.2, 19.7, 15.7; HRMS-ESI (m/z): [M+H]⁺ calculated for C₁₆H₂₀ClN₄O₃S: 383.0945, found: 383.0937.

### Step G: methyl 2-(3-chloro-4-methyl-6,7-dihydro-5H-pyrido[2,3-c]pyridazin-8-yl)-5-[3-[4-[3-(dimethylamino)prop-1-ynyl]phenoxy]propyl]thiazole-4-carboxylate

Using Mitsunobu General Procedure I starting from 0.65 g (1.2 eq) of the product from Step F and 250 mg (1.43 mmol) of 4-[3-(dimethylamino)prop-1-ynyl]phenol in THF (9 mL/mmol), 0.28 g (37%) of the desired product was obtained. ¹H NMR (500 MHz, DMSO-d₆) δ ppm 7.34 (d, 2H), 6.91 (d, 2H), 4.26 (t, 2H), 4.03 (t, 2H), 3.78 (s, 3H), 3.40 (s, 2H), 3.25 (t, 2H), 2.88 (t, 2H), 2.31 (s, 3H), 2.22 (s, 6H), 2.08 (qn, 2H), 2.03 (qn, 2H); ¹³C NMR (125 MHz, DMSO-d₆) δ ppm 163.1, 158.9, 155.3, 151.7, 151.3, 142.7, 136.2, 134.9, 133.3, 129.0, 115.2, 115.0, 85.2, 84.1, 67.1, 52.0, 48.3, 46.3, 44.3, 30.8, 24.1, 23.1, 19.7, 15.7; HRMS-ESI (m/z): [M+H]⁺ calculated for C₂₇H₃₁ClN₅O₃S: 540.1836, found: 540.1834.

### Step H: methyl 2-[3-(1,3-benzothiazol-2-ylamino)-4-methyl-6,7-dihydro-5H-pyrido[2,3-c]pyridazin-8-yl]-5-[3-[4-[3-(dimethylamino)prop-1-ynyl]phenoxy]propyl]thiazole-4-carboxylate

Using Buchwald General Procedure I starting from 0.27 g of the product from Step G (0.5 mmol), 0.29 g (89%) of the desired product was obtained. ¹H NMR (500 MHz, DMSO-d₆) ppm 7.83 (dm, 1H), 7.50 (dm, 1H), 7.36 (m, 1H), 7.35 (m, 2H), 7.18 (m, 1H), 6.94 (m, 2H), 4.28 (m, 2H), 4.09 (t, 2H), 3.80 (s, 3H), 3.39 (s, 2H), 3.29 (t, 2H), 2.88 (t, 2H), 2.35 (s, 3H), 2.23 (s, 6H), 2.13 (m, 2H), 2.07 (m, 2H); HRMS-ESI (m/z): [M+H]⁺ calculated for C₃₄H₃₆N₇O₃S₂: 654.2321, found: 654.2322.

### Step I: 2-[3-(1,3-benzothiazol-2-ylamino)-4-methyl-6,7-dihydro-5H-pyrido[2,3-c]pyridazin-8-yl]-5-[3-[4-[3-(dimethylamino)prop-1-ynyl]phenoxy]propyl]thiazole-4-carboxylic acid

To the product from Step H (280 mg, 0.43 mmol) in a 1:1 mixture of THF and water (10 mL/mmol) was added 90 mg (5 eq) of LiOH×H₂O, and the reaction mixture was stirred at 50° C. for 18 h. After the removal of the volatiles, purificationby reverse phase preparative chromatography (C18, 0.1% TFA in water and MeCN as eluents) afforded 132 mg (48%) of the desired compound. HRMS-ESI (m/z): [M+H]⁺ calculated for C₃₃H₃₄N₇O₃S₂: 640.2165, found: 640.2160.

### Preparation of P29: 6-[[6-(1,3-benzothiazol-2-ylamino)-5-methyl-pyridazin-3-yl]-methyl-amino]-3-[1-[[3-[2-(3-methoxypropylamino)ethoxy]-5,7-dimethyl-1-adamantyl]methyl]-5-methyl-pyrazol-4-yl]pyridine-2-carboxylic acid

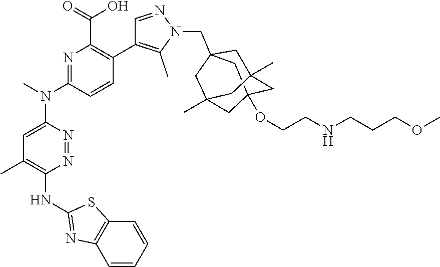

Using the Amine Substitution and Hydrolysis General procedure II starting from Preparation 14_01 and 3-methoxypropan-1-amine as the appropriate amine, the desired product was obtained. HRMS-ESI (m/z): [M+H]⁺ calculated for C₄₂H₅₄N₉O₄S: 780.4019, found: 780.4019.

### Preparation of P30: 6-[{6-[(1,3-benzothiazol-2-yl)amino]-5-methylpyridazin-3-yl}(methyl)amino]-3-[1-({3-[2-(dimethylamino)ethoxy]-5,7-dimethyladamantan-1-yl}methyl)-5-methyl-1H-pyrazol-4-yl]pyridine-2-carboxylic acid

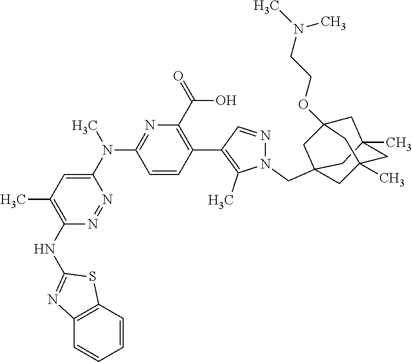

Using the Amine Substitution and Hydrolysis General procedure II starting from Preparation 14_01 and dimethylamine as the appropriate amine, the desired product was obtained. HRMS-ESI (m/z): [M+H]⁺ calculated for C₄₀H₅₀N₉O₃S: 736.3757, found: 736.3751.

### Preparation of P31: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-piperazin-1-ylethoxy)-1-adamantyl]methyl]-5-methyl-pyrazol-4-yl]pyridine-2-carboxylic acid

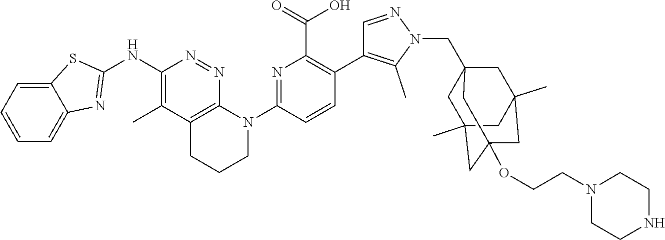 [SMILES: Cc1c(Nc2nc3ccccc3s2)nnc2c1CCCN2c1ccc(-c2cnn(CC34CC5(C)CC(C)(C3)CC(OCCN3CCNCC3)(C5)C4)c2C)c(C(=O)O)n1]

Using the Amine substitution and Hydrolysis General procedure I starting from Preparation 12 and piperazine as the appropriate amine, the desired product was obtained. HRMS-ESI (m/z): [M+H]⁺ calculated for C₄₄H₅₅N₁₀O₃S: 803.4179, found: 803.4177.

### Preparation of P32: 6-[3-(1,3-benzothiazol-2-ylamino)-4-methyl-6,7-dihydro-5H-pyrido[2,3-c]pyridazin-8-yl]-3-[1-[[3-[2-(4-isopropylpiperazin-1-yl)ethoxy]-5,7-dimethyl-1-adamantyl]methyl]-5-methyl-pyrazol-4-yl]pyridine-2-carboxylic acid

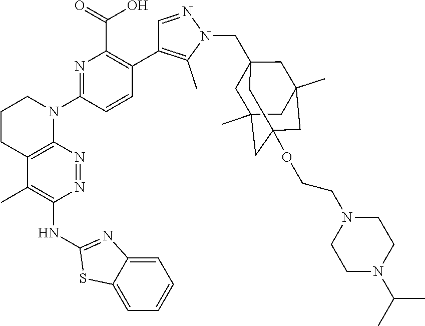 [SMILES: Cc1c(Nc2nc3ccccc3s2)nnc2c1CCCN2c1ccc(-c2cnn(CC34CC5(C)CC(C)(C3)CC(OCCN3CCN(C(C)C)CC3)(C5)C4)c2C)c(C(=O)O)n1]

Using the Amine substitution and Hydrolysis General procedure I starting from Preparation 12 and 1-isopropylpiperazine as the appropriate amine, the desired product was obtained. HRMS-ESI (m/z): [M+H]⁺ calculated for C₄₇H₆₁N₁₀O₃S: 845.4649, found: 845.4646.

### Preparation of P33: 3-[1-[[3-[2-(azepan-1-yl)ethoxy]-5,7-dimethyl-1-adamantyl]methyl]-5-methyl-pyrazol-4-yl]-6-[3-(1,3-benzothiazol-2-ylamino)-4-methyl-6,7-dihydro-5H-pyrido[2,3-c]pyridazin-8-yl]pyridine-2-carboxylic acid

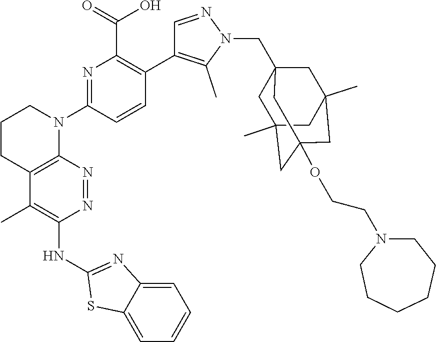 [SMILES: Cc1c(Nc2nc3ccccc3s2)nnc2c1CCCN2c1ccc(-c2cnn(CC34CC5(C)CC(C)(C3)CC(OCCN3CCCCCC3)(C5)C4)c2C)c(C(=O)O)n1]

Using the Amine substitution and Hydrolysis General procedure I starting from Preparation 12 and azepane as the appropriate amine, the desired product was obtained. HRMS-ESI (m/z): [M+H]⁺ calculated for C₄₆H₅₈N₉O₃S: 816.4383, found: 816.4379.

### Preparation of P34: 2-[3-(1,3-benzothiazol-2-ylamino)-4-methyl-6,7-dihydro-5H-pyrido[2,3-c]pyridazin-8-yl]-5-[3-[4-[3-[[(3S)-3,4-dihydroxybutyl]-methyl-amino]prop-1-ynyl]-2-fluoro-phenoxy]propyl]thiazole-4-carboxylic acid

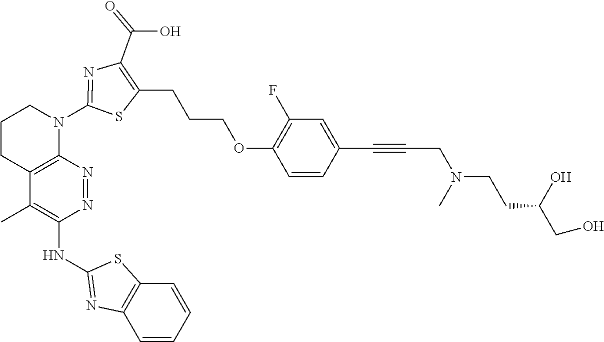 [SMILES: Cc1c(Nc2nc3ccccc3s2)nnc2c1CCCN2c1nc(C(=O)O)c(CCCOc2ccc(C#CCN(C)CC[C@H](O)CO)cc2F)s1]

### Step A: 2-[(4S)-2,2-dimethyl-1,3-dioxolan-4-yl]ethyl 4-methylbenzenesulfonate

To 1.0 g (6.8 mmol) of 2-[(4S)-2,2-dimethyl-1,3-dioxolan-4-yl]ethanol and 3.8 mL (4 eq) of triethylamine in 34 mL of DCM was added 4.5 g (2 eq) of p-tolylsulfonyl 4-methylbenzenesulfonate at 0° C. The reaction mixture was stirred until no further conversion was observed, concentrated and treated with diisopropyl ether. Then, the precipitated hydrochloric salt was filtered off and the mother liquour was concentrated and purified via flash chromatography (silica gel, using heptane and EtOAc as eluents) to give 1.6 g (81%) of desired product. ¹H NMR (500 MHz, dmso-d₆) δ ppm 7.79 (dm, 2H), 7.49 (dm, 2H), 4.08 (m, 2H), 4.00 (m, 1H), 3.91/3.44 (dd+dd, 2H), 2.42 (s, 3H), 1.83/1.77 (m+m, 2H), 1.24/1.20 (s+s, 6H); 13C NMR (500 MHz, dmso-d₆) δ ppm 132.7, 132.7, 130.7, 128.1, 108.6, 72.3, 68.7, 68.4, 32.9, 27.2/25.9, 21.6; HRMS-ESI (m/z): [M+H]⁺ calculated for C14H21O5S: 301.1110, found: 301.1107.

### Step B: N-[2-[(4S)-2,2-dimethyl-1,3-dioxolan-4-yl]ethyl]prop-2-yn-1-amine

The mixture of the product from Step A (7.6 g, 25.3 mmol), prop-2-yn-1-amine (16 mL, 10 eq) and DIPEA (13.22 mL, 3 eq) in 127 mL of MeCN was stirred at 50° C. for 16 h. After concentration, taken up in DCM and extraction with cc. NaHCO₃ solution and brine, the combined organic layers were dried and concentrated to give 5.0 g (107%) of the desired product, which was used without any further purification. ¹H NMR (500 MHz, dmso-d₆) δ ppm 4.07 (m, 1H), 3.98/3.43 (dd+t, 2H), 3.28 (m, 2H), 3.05 (t, 1H), 2.62/2.55 (m+m, 2H), 2.23 (brs, 1H), 1.63/1.59 (m+m, 2H), 1.30 (s, 3H), 1.25 (s, 3H); ¹³C NMR (500 MHz, dmso-d₆) δ ppm 108.2, 83.4, 74.6, 74.1, 69.2, 45.1, 37.8, 33.6, 27.3, 26.2; HRMS (EI)(m/z): [M]⁺ calculated for C₁₀H₁₇NO₂: 183.1259, found: 183.1260.

### Step C: N-[2-[(4S)-2,2-dimethyl-1,3-dioxolan-4-yl]ethyl]-N-methyl-prop-2-yn-1-amine

To the product from Step B (500 mg, 2.73 mmol) in N,N-dimethylformamide (14 mL) was added portionwise sodium hydride (120 mg, 1.1 eq) at 0° C. After stirring at 0° C. for 0.5 h, the mixture was treated with iodomethane (0.17 mL, 1 eq) and stirred at rt for 18 h. After quenching with a saturated solution of NH₄Cl and water, the mixture was extracted with Et₂O. The combined organic phases were dried and concentrated to give the desired product (362 mg, 67%). GC/MS (C₁₁H₁₉NO₂) 197 [M+].

### Step D: ethyl 2-(3-chloro-4-methyl-6,7-dihydro-5H-pyrido[2,3-c]pyridazin-8-yl)-5-[3-[4-[3-[2-[(4S)-2,2-dimethyl-1,3-dioxolan-4-yl]ethyl-methyl-amino]prop-1-ynyl]-2-fluoro-phenoxy]propyl]thiazole-4-carboxylate

Using Sonogashira General Procedure starting from 0.548 g (0.89 mmol) of the product of Preparation 15 and 350 mg (2 eq) of the product from Step C as the appropriate acetylene, 510 mg (82%) of the desired product was obtained. LC/MS (C₃₄H₄₂ClFN₅O₅S) 686 [M+H]⁺.

### Step E: ethyl 2-[3-(1,3-benzothiazol-2-ylamino)-4-methyl-6,7-dihydro-5H-pyrido[2,3-c]pyridazin-8-yl]-5-[3-[4-[3-[2-[(4S)-2,2-dimethyl-1,3-dioxolan-4-yl]ethyl-methyl-amino]prop-1-ynyl]-2-fluoro-phenoxy]propyl]thiazole-4-carboxylate

Using Buchwald General Procedure I starting from 510 mg (0.52 mmol) of the product from Step D and 234 mg (3 eq) of 1,3-benzothiazol-2-amine, 200 mg (48%) of the desired product was obtained. ¹H NMR (500 MHz, dmso-d₆) δ ppm 7.88 (dm, 1H), 7.49 (brd, 1H), 7.37 (m, 1H), 7.3 (dd, 1H), 7.20 (dm, 1H), 7.19 (m, 1H), 7.16 (t, 1H), 4.26 (m, 2H), 4.25 (q, 2H), 4.14 (t, 2H), 4.04 (m, 1H), 3.98/3.45 (dd+dd, 2H), 3.46 (s, 2H), 3.28 (m, 2H), 2.87 (t, 2H), 2.45/2.39 (m+m, 2H), 2.34 (s, 3H), 2.21 (s, 3H), 2.13 (m, 2H), 2.04 (m, 2H), 1.63 (m, 2H), 1.29 (t, 3H), 1.29 (s, 3H), 1.24 (s, 3H); HRMS (ESI)(m/z): [M+H]⁺ calculated for C₄₁H₄₇FN₇O₅S₂: 800.3064, found: 800.3064.

### Step F: 2-[3-(1,3-benzothiazol-2-ylamino)-4-methyl-6,7-dihydro-5H-pyrido[2,3-c]pyridazin-8-yl]-5-[3-[4-[3-[[(3S)-3,4-dihydroxybutyl]-methyl-amino]prop-1-ynyl]-2-fluoro-phenoxy]propyl]thiazole-4-carboxylic acid

The mixture of 200 mg (0.25 mmol) of product from Step E and 53 mg of LiOH×H₂O (5 eq) in 5 mL of THF/water (1:1) was stirred at 60° C. for 18 h. The reaction mixture was treated with 0.125 mL (6 eq) of concentrated hydrogen chloride at 0° C. (pH=2-3) and stirred at rt, then at 60° C. for 0.5 h. After the reaction mixture was concentrated to remove THF and lyophilization, the solid was dissolved in 6 N NH₃ solution in MeOH and purified by reverse phase chromatography (using 5 mM NH₄HCO₃ and MeCN as eluents) to give 47 mg (25%) of the desired product. HRMS (ESI)(m/z): [M+H]⁺ calculated for C₃₆H₃₉FN₇O₅S₂: 732.2438, found: 732.2441.

### Preparation of P35: 6-[3-(1,3-benzothiazol-2-ylamino)-4-methyl-6,7-dihydro-5H-pyrido[2,3-c]pyridazin-8-yl]-3-[1-[[3-[2-[2-hydroxyethyl(methyl)amino]ethoxy]-5,7-dimethyl-1-adamantyl]methyl]-5-methyl-pyrazol-4-yl]pyridine-2-carboxylic acid

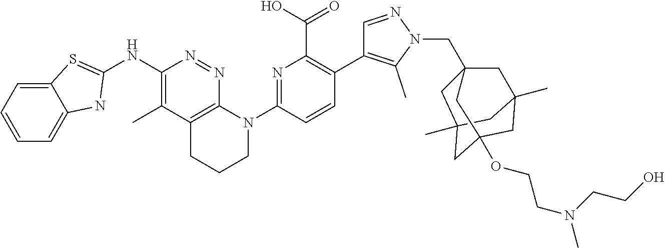

Using the Amine substitution and Hydrolysis General procedure I starting from Preparation 12 and 2-(methylamino) ethanol as the appropriate amine, the desired product was obtained. HRMS-ESI (m/z): [M+H]⁺ calculated for C₄₃H₅₄N₉O₄S: 792.4019, found: 792.4019.

### Preparation of P36: 6-[3-(1,3-benzothiazol-2-ylamino)-4-methyl-6,7-dihydro-5H-pyrido[2,3-c]pyridazin-8-yl]-3-[1-[[3-[2-[3-methoxypropyl(methyl)amino]ethoxy]-5,7-dimethyl-1-adamantyl]methyl]-5-methyl-pyrazol-4-yl]pyridine-2-carboxylic acid

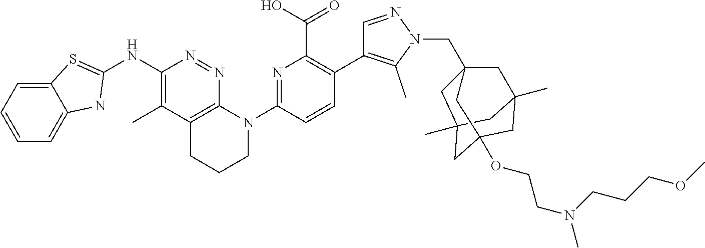

Using the Amine substitution and Hydrolysis General procedure I starting from Preparation 12 and 3-methoxy-N-methyl-propan-1-amine as the appropriate amine, the desired product was obtained. HRMS-ESI (m/z): [M+H]⁺ calculated for C₄₅H₅₈N₉O₄S: 820.4332, found: 820.4328.

### Preparation of P37: 6-[3-(1,3-benzothiazol-2-ylamino)-4-methyl-6,7-dihydro-5H-pyrido[2,3-c]pyridazin-8-yl]-3-[1-[[3-[2-[4-hydroxybutyl(methyl)amino]ethoxy]-5,7-dimethyl-1-adamantyl]methyl]-5-methyl-pyrazol-4-yl]pyridine-2-carboxylic acid

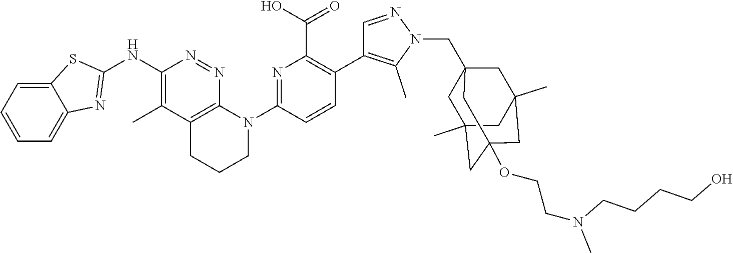 [SMILES: Cc1c(Nc2nc3ccccc3s2)nnc2c1CCCN2c1ccc(-c2cnn(CC34CC5(C)CC(C)(C3)CC(OCCN(C)CCCCO)(C5)C4)c2C)c(C(=O)O)n1]

Using the Amine substitution and Hydrolysis General procedure I starting from Preparation 12 and 4-(methylamino) butan-1-ol as the appropriate amine, the desired product was obtained. HRMS-ESI (m/z): [M+H]⁺ calculated for C₄₅H₅₈N₉O₄S: 820.4332, found: 820.4339.

### Preparation of P38: 6-[3-(1,3-benzothiazol-2-ylamino)-4-methyl-6,7-dihydro-5H-pyrido[2,3-c]pyridazin-8-yl]-3-[1-[[3,5-dimethyl-7-[2-(1-piperidyl)ethoxy]-1-adamantyl]methyl]-5-methyl-pyrazol-4-yl]pyridine-2-carboxylic acid

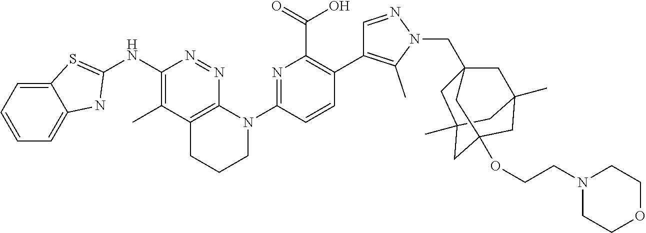

Using the Amine substitution and Hydrolysis General procedure I starting from Preparation 12 and piperidine as the appropriate amine, the desired product was obtained. HRMS-ESI (m/z): [M+H]⁺ calculated for C₄₅H₅₆N₉O₃S: 802.4227, found: 802.4223.

### Preparation of P39: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-morpholinoethoxy)-1-adamantyl]methyl]-5-methyl-pyrazol-4-yl]pyridine-2-carboxylic acid

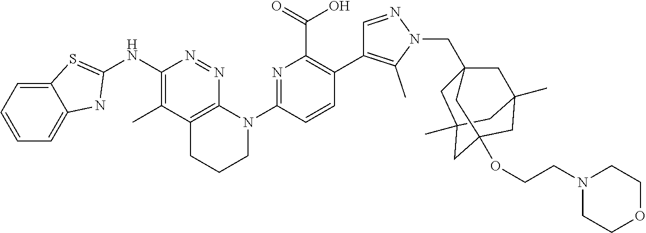

Using the Amine substitution and Hydrolysis General procedure I starting from Preparation 12 and morpholine as the appropriate amine, the desired product was obtained. HRMS-ESI (m/z): [M+H]⁺ calculated for C₄₄H₅₄N₉O₄S: 804.4019, found: 804.4012.

### Preparation of P40: 6-[3-(1,3-benzothiazol-2-ylamino)-4-methyl-6,7-dihydro-5H-pyrido[2,3-c]pyridazin-8-yl]-3-[1-[[3-[3-(3-hydroxypropylamino)propyl]-5,7-dimethyl-1-adamantyl]methyl]-5-methyl-pyrazol-4-yl]pyridine-2-carboxylic acid

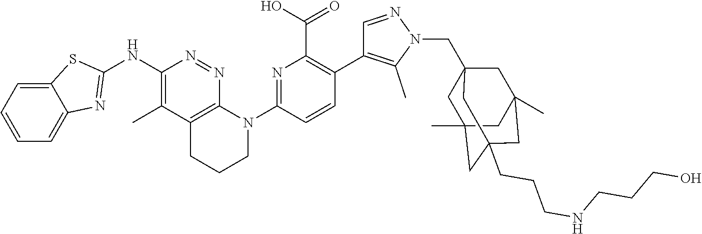 [SMILES: Cc1c(Nc2nc3ccccc3s2)nnc2c1CCCN2c1ccc(-c2cnn(CC34CC5(C)CC(C)(CC(CCCNCCCO)(C5)C3)C4)c2C)c(C(=O)O)n1]

Using the Amine substitution and Hydrolysis General procedure I starting from Preparation 13 and 3-aminopropan-1-ol as the appropriate amine, the desired product was obtained. HRMS-ESI (m/z): [M+H]⁺ calculated for C₄₄H₅₆N₉O₃S: 790.4227, found: 790.4220.

### Preparation of P41: 6-[3-(1,3-benzothiazol-2-ylamino)-4-methyl-6,7-dihydro-5H-pyrido[2,3-c]pyridazin-8-yl]-3-[1-[[3,5-dimethyl-7-(3-pyrrolidin-1-ylpropyl)-1-adamantyl]methyl]-5-methyl-pyrazol-4-yl]pyridine-2-carboxylic acid

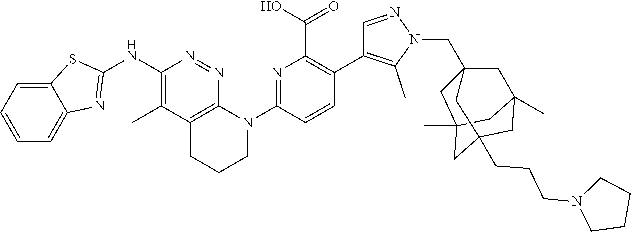 [SMILES: Cc1c(Nc2nc3ccccc3s2)nnc2c1CCCN2c1ccc(-c2cnn(CC34CC5(C)CC(C)(CC(CCCN6CCCC6)(C5)C3)C4)c2C)c(C(=O)O)n1]

Using the Amine substitution and Hydrolysis General procedure I starting from Preparation 13 and pyrrolidine as the appropriate amine, the desired product was obtained. HRMS-ESI (m/z): [M+H]⁺ calculated for C₄₅H₅₆N₉O₂S: 786.4278, found: 786.4273.

### Preparation of P42: 3-[1-[[3,5-dimethyl-7-(2-pyrrolidin-1-ylethoxy)-1-adamantyl]methyl]-5-methyl-pyrazol-4-yl]-6-[4-methyl-3-[(5-methyl-1,3-benzothiazol-2-yl)amino]-6,7-dihydro-5H-pyrido[2,3-c]pyridazin-8-yl]pyridine-2-carboxylic acid

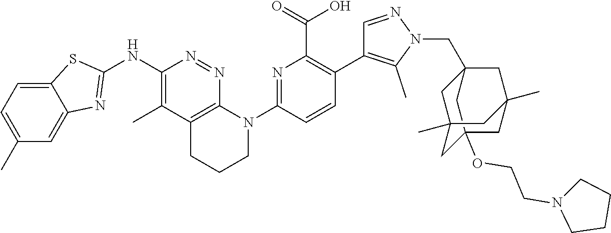 [SMILES: Cc1ccc2sc(Nc3nnc4c(c3C)CCCN4c3ccc(-c4cnn(CC56CC7(C)CC(C)(C5)CC(OCCN5CCCC5)(C7)C6)c4C)c(C(=O)O)n3)nc2c1]

### Step A: methyl 3-[1-[[3-(2-hydroxyethoxy)-5,7-dimethyl-1-adamantyl]methyl]-5-methyl-pyrazol-4-yl]-6-[4-methyl-3-[(5-methyl-1,3-benzothiazol-2-yl)amino]-6,7-dihydro-5H-pyrido[2,3-c]pyridazin-8-yl]pyridine-2-carboxylate

Using Buchwald General Procedure I at 130° C. for 1.5 h, starting from 140 mg (0.22 mmol) of the product from Preparation 12, Step C and 54.3 mg (1.5 eq) of the 5-methyl-1,3-benzothiazol-2-amine, 126 mg (75%) of the desired product was obtained. ¹H NMR (500 MHz, dmso-d₆) δ ppm 12.08/10.89 (brs/brs, 1H), 7.95 (d, 1H), 7.69 (d, 1H), 7.67 (br, 1H), 7.38 (s, 1H), 7.30 (br, 1H), 7.00 (d, 1H), 4.46 (brs, 1H), 4.00 (t, 2H), 3.88 (s, 2H), 3.70 (s, 3H), 3.41 (t, 2H), 3.35 (t, 2H), 2.85 (t, 2H), 2.39 (s, 3H), 2.32 (s, 3H), 2.16 (s, 3H), 1.98 (qn, 2H), 1.39 (s, 2H), 1.30/1.25 (d+d, 4H), 1.18/1.12 (d+d, 4H), 1.08/1.02 (d+d, 2H), 0.87 (s, 6H); ¹³C NMR (500 MHz, dmso-d₆) δ ppm 139.8, 137.5, 123.6, 121.6, 119.0, 62.1, 61.5, 59.0, 52.7, 50.1, 47.0, 46.0, 45.4, 43.3, 30.2, 24.3, 21.7, 21.6, 12.6, 10.9; HRMS-ESI (m/z): [M+H]⁺ calculated for C₄₂H₅₁N₈O₄S: 763.3760, found: 763.3754.

### Step B: methyl 3-[1-[[3,5-dimethyl-7-[2-(p-tolylsulfonyloxy)ethoxy]-1-adamantyl]methyl]-5-methyl-pyrazol-4-yl]-6-[4-methyl-3-[(5-methyl-1,3-benzothiazol-2-yl)amino]-6,7-dihydro-5H-pyrido[2,3-c]pyridazin-8-yl]pyridine-2-carboxylate

To the product from Step A (119 mg, 0.16 mmol) and triethylamine (0.066 mL, 3 eq) in DCM (2 mL) was added p-tolylsulfonyl 4-methylbenzenesulfonate (76 mg, 1.5 eq) and the reaction mixture was stirred for 1 h. Purification by column chromatography (silica gel, DCM and EtOAc as eluents) afforded the desired product (93 mg, 65%). ¹H NMR (500 MHz, dmso-d₆) δ ppm 12.17/10.83 (brs/brs, 1H), 7.95 (d, 1H), 7.77 (d, 2H), 7.7 (d, 1H), 7.69 (br, 1H), 7.46 (d, 2H), 7.42 (br, 1H), 7.39 (s, 1H), 7.00 (d, 1H), 4.07 (t, 2H), 4 (t, 2H), 3.96 (s, 3H), 3.85 (s, 2H), 3.49 (t, 2H), 2.85 (t, 2H), 2.40 (s, 3H), 2.39 (s, 3H), 2.32 (s, 3H), 2.15 (s, 3H), 1.99 (qn, 2H), 1.29 (s, 2H), 1.17/1.1 (d+d, 4H), 1.12/1.1 (d+d, 4H), 1.02/0.97 (d+d, 2H), 0.84 (s, 6H); ¹³C NMR (500 MHz, dmso-d₆) δ ppm 139.8, 137.6, 130.6, 128.1, 123.6, 119.0, 71.5, 58.8, 58.4, 52.7, 49.9, 46.6, 45.9, 45.4, 43.0, 30.1, 24.3, 21.6, 21.6, 21.6, 12.6, 10.9; HRMS-ESI (m/z): [M+H]⁺ calculated for C₄₉H₅₇N₈O₆S₂: 917.3842, found: 917.3840.

### Step C: 3-[1-[[3,5-dimethyl-7-(2-pyrrolidin-1-ylethoxy)-1-adamantyl]methyl]-5-methyl-pyrazol-4-yl]-6-[4-methyl-3-[(5-methyl-1,3-benzothiazol-2-yl)amino]-6,7-dihydro-5H-pyrido[2,3-c]pyridazin-8-yl]pyridine-2-carboxylic acid

Using the Amine substitution and Hydrolysis General procedure I starting from the product from Step B and pyrrolidine as the appropriate amine, the desired product was obtained. HRMS-ESI (m/z): [M+H]⁺ calculated for C₄₅H₅₆N₉O₃S: 802.4227, found: 802.4220.

### Preparation of P43: 3-[1-[[3,5-dimethyl-7-(2-pyrrolidin-1-ylethoxy)-1-adamantyl]methyl]-5-methyl-pyrazol-4-yl]-6-[3-[(5-methoxy-1,3-benzothiazol-2-yl)amino]-4-methyl-6,7-dihydro-5H-pyrido[2,3-c]pyridazin-8-yl]pyridine-2-carboxylic acid

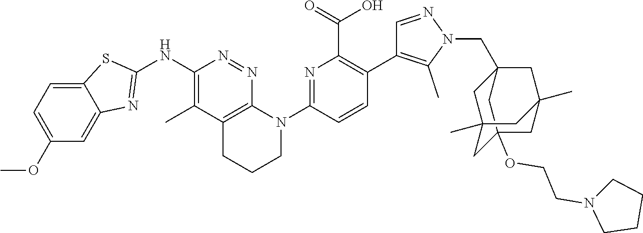 [SMILES: COc1ccc2sc(Nc3nnc4c(c3C)CCCN4c3ccc(-c4cnn(CC56CC7(C)CC(C)(C5)CC(OCCN5CCCC5)(C7)C6)c4C)c(C(=O)O)n3)nc2c1]

### Step A: methyl 3-[1-[[3-(2-hydroxyethoxy)-5,7-dimethyl-1-adamantyl]methyl]-5-methyl-pyrazol-4-yl]-6-[3-[(5-methoxy-1,3-benzothiazol-2-yl)amino]-4-methyl-6,7-dihydro-5H-pyrido[2,3-c]pyridazin-8-yl]pyridine-2-carboxylate

Using Buchwald General Procedure I at 130° C. for 2.5 h, starting from 140 mg (0.22 mmol) of the product from Preparation 12, Step C and 60 mg (1.5 eq) of the 5-methyl-1,3-benzothiazol-2-amine, 129 mg (75%) of the desired product was obtained. ¹H NMR (500 MHz, dmso-d₆) δ ppm 7.95 (d, 1H), 7.69 (d, 1H), 7.67 (br., 1H), 7.38 (s, 1H), 7.02 (br., 1H), 6.80 (dd, 1H), 4.46 (br., 1H), 4.00 (t, 2H), 3.88 (s, 2H), 3.80 (s, 3H), 3.70 (s, 3H), 3.41 (t, 2H), 3.35 (t, 2H), 2.85 (t, 2H), 2.32 (s, 3H), 2.16 (s, 3H), 1.98 (m, 2H), 1.39 (s, 2H), 1.30/1.25 (d+d, 4H), 1.18/1.12 (d+d, 4H), 1.08/1 (d+d, 2H), 0.87 (s, 6H); ¹³C NMR (500 MHz, dmso-d₆) δ ppm 139.8, 137.5, 122.6, 119.0, 110.5, 62.1, 61.5, 58.9, 55.8, 52.6, 50.1, 47.0, 46.0, 45.4, 43.3, 30.2, 24.3, 21.7, 12.6, 10.9; HRMS-ESI (m/z): [M+H]⁺ calculated for C₄₂H₅₁N₈O₅S: 779.3703, found: 779.3687.

### Step B: methyl 3-[1-[[3,5-dimethyl-7-[2-(p-tolylsulfonyloxy)ethoxy]-1-adamantyl]methyl]-5-methyl-pyrazol-4-yl]-6-[3-[(5-methoxy-1,3-benzothiazol-2-yl)amino]-4-methyl-6,7-dihydro-5H-pyrido[2,3-c]pyridazin-8-yl]pyridine-2-carboxylate

To the product from Step A (122 mg, 0.16 mmol) and triethylamine (0.066 mL, 3 eq) in DCM (2 mL) was added p-tolylsulfonyl 4-methylbenzenesulfonate (77 mg, 1.5 eq) and the reaction mixture was stirred for 1 h. Purification by column chromatography (silica gel, DCM and EtOAc as eluents) afforded the desired product (79 mg, 54%). ¹H NMR (500 MHz, dmso-d₆) δ ppm 12.17/10.83 (brs/brs, 1H), 7.95 (d, 1H), 7.77 (d, 2H), 7.72 (d, 1H), 7.67 (brd, 1H), 7.46 (d, 2H), 7.39 (s, 1H), 7.02 (br, 1H), 6.80 (d, 1H), 4.07 (t, 2H), 4.00 (t, 2H), 3.86 (s, 2H), 3.80 (s, 3H), 3.69 (s, 3H), 3.49 (t, 2H), 2.86 (t, 2H), 2.41 (s, 3H), 2.33 (s, 3H), 2.15 (s, 3H), 1.99 (qn, 2H), 1.29 (s, 2H), 1.17/1.1 (d+d, 4H), 1.12/1.10 (d+d, 4H), 1.02/0.97 (d+d, 2H), 0.84 (s, 6H); 13C NMR (500 MHz, dmso-d₆) δ ppm 139.9, 137.6, 130.6, 128.1, 119.0, 110.6, 71.5, 58.8, 58.4, 55.9, 52.6, 49.9, 46.6, 45.9, 45.8, 43.0, 30.1, 24.3, 21.6, 21.6, 12.7, 10.9; HRMS-ESI (m/z): [M+H]⁺ calculated for C₄₉H₅₇N₈O₇S₂: 933.3792, found: 933.3794.

### Step C: 3-[1-[[3,5-dimethyl-7-(2-pyrrolidin-1-ylethoxy)-1-adamantyl]methyl]-5-methyl-pyrazol-4-yl]-6-[3-[(5-methoxy-1,3-benzothiazol-2-yl)amino]-4-methyl-6,7-dihydro-5H-pyrido[2,3-c]pyridazin-8-yl]pyridine-2-carboxylic acid

Using the Amine substitution and Hydrolysis General procedure I starting from the product from Step B and pyrrolidine as the appropriate amine, the desired product was obtained. HRMS-ESI (m/z): [M+H]⁺ calculated for C₄₅H₅₇N₉O₄S: 818.4176, found: 818.4172.

### Preparation of P44: 2-[[6-(1,3-Benzothiazol-2-ylamino)-5-methyl-pyridazin-3-yl]-(3,4-dihydroxybutyl)amino]-5-[3-[2-fluoro-4-[3-(methylamino)prop-1-ynyl]phenoxy]propyl]thiazole-4-carboxylic acid

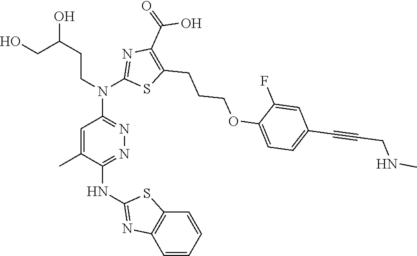 [SMILES: CNCC#Cc1ccc(OCCCc2sc(N(CCC(O)CO)c3cc(C)c(Nc4nc5ccccc5s4)nn3)nc2C(=O)O)c(F)c1]

### Step A: methyl 5-[3-[4-[3-[tert-butoxycarbonyl(methyl)amino]prop-1-ynyl]-2-fluoro-phenoxy]propyl]-2-[2-(2,2-dimethyl-1,3-dioxolan-4-yl)ethyl-[5-methyl-6-[(Z)-[3-(2-trimethylsilylethoxymethyl)-1,3-benzothiazol-2-ylidene]amino]pyridazin-3-yl]amino]thiazole-4-carboxylate

Using Buchwald General Procedure III starting from 350 mg of Preparation 3h_01 (0.57 mmol, 1 eq.) and 235 mg of Preparation 4a_01 (0.57 mmol, 1 eq.) as the appropriate halide, 490 mg (87%) of the desired product was obtained. ¹H NMR (500 MHz, DMSO-d₆) δ ppm 7.84 (d, 1H), 7.68 (s, 1H), 7.47 (d, 1H), 7.44 (td, 1H), 7.32 (brd., 1H), 7.25 (td, 1H), 7.22 (d, 1H), 7.16 (t, 1H), 5.86 (s, 2H), 4.49/4.33 (m+m, 2H), 4.20 (br., 2H), 4.17 (m, 1H), 4.15 (t, 2H), 4.04/3.63 (dd+dd, 2H), 3.77 (s, 3H), 3.72 (t, 2H), 3.27 (t, 2H), 2.84 (br., 3H), 2.45 (s, 3H), 2.13 (m, 2H), 1.75 (m, 2H), 1.40 (s, 9H), 1.37/1.24 (s+s, 6H), 0.92 (t, 2H), −0.11 (s, 9H); ¹³C NMR (125 MHz, DMSO-d₆) δ ppm 129.1, 127.2, 123.5, 123.2, 119.3, 117.5, 115.5, 112.0, 108.6, 73.7, 72.8, 68.9, 68.4, 66.7, 51.9, 44.4, 38.5, 33.8, 30.9, 28.5, 27.3/26.0, 23.3, 23.1, 17.9, 17.8, −1.0; HRMS-ESI (m/z): [M+H]⁺ calculated for C₄₈H₆₃FN₇O₈S₂Si: 976.3927, found 976.3916.

### Step B: 2-[[6-(1,3-benzothiazol-2-ylamino)-5-methyl-pyridazin-3-yl]-(3,4-dihydroxybutyl)amino]-5-[3-[2-fluoro-4-[3-(methylamino)prop-1-ynyl]phenoxy]propyl]thiazole-4-carboxylic acid

Using Deprotection and Hydrolysis General Procedure starting from the product from Step A as the appropriate methyl ester, the desired product was obtained. HRMS-ESI (m/z): [M+H]⁺ calculated for C₃₃H₃₅FN₇O₅S₂: 692.2120, found 692.2114.

### Preparation of P45: 2-[[6-(1,3-Benzothiazol-2-ylamino)-5-methyl-pyridazin-3-yl]-(3-hydroxypropyl)amino]-5-[3-[2-fluoro-4-[3-(methylamino)prop-1-ynyl]phenoxy]propyl]thiazole-4-carboxylic acid

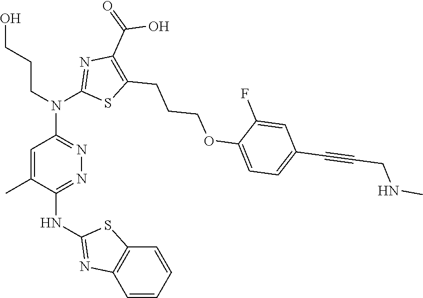

### Step A: methyl 5-[3-[4-[3-[tert-butoxycarbonyl(methyl)amino]prop-1-ynyl]-2-fluoro-phenoxy]propyl]-2-[3-[tert-butyl(dimethyl)silyl]oxypropyl-[5-methyl-6-[(Z)-[3-(2-trimethylsilylethoxymethyl)-1,3-benzothiazol-2-ylidene]amino]pyridazin-3-yl]amino]thiazole-4-carboxylate

Using Buchwald General Procedure III starting from 300 mg of Preparation 3n_01 (0.46 mmol, 1 eq.) and 187 mg of Preparation 4a_01 (0.46 mmol, 1 eq.) as the appropriate halide, 395 mg (83%) of the desired product was obtained. ¹H NMR (500 MHz, DMSO-d₆) δ ppm 7.82 (dd, 1H), 7.60 (s, 1H), 7.44 (m, 1H), 7.44 (dd, 1H), 7.31 (dd, 1H), 7.24 (m, 1H), 7.20 (m, 1H), 7.15 (t, 1H), 5.84 (s, 2H), 4.39 (t, 2H), 4.20 (s, 2H), 4.14 (t, 2H), 3.76 (s, 3H), 3.70 (t, 2H), 3.70 (t, 2H), 3.25 (t, 2H), 2.84 (s, 3H), 2.42 (s, 3H), 2.11 (m, 2H), 1.91 (m, 2H), 1.40 (s, 9H), 0.91 (t, 2H), 0.85 (s, 9H), 0.01 (s, 6H), −0.12 (s, 9H); ¹³C NMR (125 MHz, DMSO-d₆) δ ppm 162.2, 147.5, 137.6, 129.1, 127.2, 123.4, 123.2, 119.3, 117.5, 115.4, 112.0, 79.7, 72.8, 68.4, 66.7, 60.5, 51.9, 44.6, 38.1, 33.8, 30.9, 30.4, 28.6, 26.3, 23.1, 17.9, 17.8, −0.9, −5.0; HRMS-ESI (m/z): [M+H]⁺ calculated for C₅₀H₇₁FN₇O₇S₂Si₂: 1020.4373, found 1020.4365.

### Step B: 2-[[6-(1,3-benzothiazol-2-ylamino)-5-methyl-pyridazin-3-yl]-(3-hydroxypropyl)amino]-5-[3-[2-fluoro-4-[3-(methylamino)prop-1-ynyl]phenoxy]propyl]thiazole-4-carboxylic acid

Using Deprotection and Hydrolysis General Procedure starting from the product from Step A as the appropriate methyl ester, the desired product was obtained. HRMS-ESI (m/z): [M+H]⁺ calculated for C₃₂H₃₃FN₇O₄S₂: 662.2014, found 662.2016.

### Preparation of P46: 2-[[6-(1,3-Benzothiazol-2-ylamino)-5-methyl-pyridazin-3-yl]-(4,5-dihydroxypentyl)amino]-5-[3-[2-fluoro-4-[3-(methylamino)prop-1-ynyl]phenoxy]propyl]thiazole-4-carboxylic acid

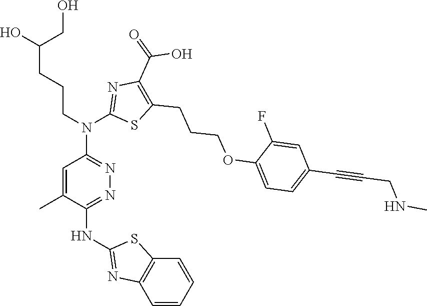 [SMILES: CNCC#Cc1ccc(OCCCc2sc(N(CCCC(O)CO)c3cc(C)c(Nc4nc5ccccc5s4)nn3)nc2C(=O)O)c(F)c1]

Using Deprotection and Hydrolysis General Procedure starting from the product from Preparation 5a_01, Step A as the appropriate methyl ester, the desired product was obtained. HRMS-ESI (m/z): [M+H]⁺ calculated for C₃₄H₃₇FN₇O₅S₂: 706.2276, found 706.2274.

### Preparation of P47: 6-[3-(1,3-benzothiazol-2-ylamino)-4-methyl-6,7-dihydro-5H-pyrido[2,3-c]pyridazin-8-yl]-3-[1-[[3-[2-(carboxymethylamino)ethoxy]-5,7-dimethyl-1-adamantyl]methyl]-5-methyl-pyrazol-4-yl]pyridine-2-carboxylic acid

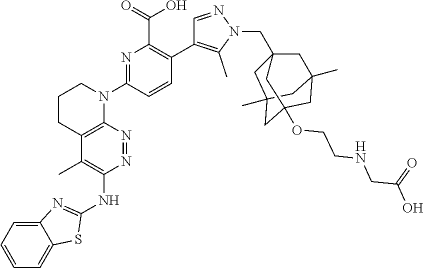 [SMILES: Cc1c(Nc2nc3ccccc3s2)nnc2c1CCCN2c1ccc(-c2cnn(CC34CC5(C)CC(C)(C3)CC(OCCNCC(=O)O)(C5)C4)c2C)c(C(=O)O)n1]

Using the Amine Substitution and Hydrolysis General procedure III starting from Preparation 16 and methyl 2-aminoacetate, hydrogen chloride (1:1) as the appropriate amine, the desired product was obtained. HRMS-ESI (m/z): [M+H]⁺ calculated for C₄₂H₅₀N₉O₅S: 792.3656, found: 792.3651.

### Preparation of P48: 6-[3-(1,3-benzothiazol-2-ylamino)-4-methyl-6,7-dihydro-5H-pyrido[2,3-c]pyridazin-8-yl]-3-[1-[[3-[2-[carboxymethyl(methyl)amino]ethoxy]-5,7-dimethyl-1-adamantyl]methyl]-5-methyl-pyrazol-4-yl]pyridine-2-carboxylic acid

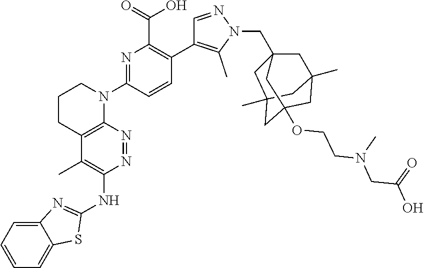 [SMILES: Cc1c(Nc2nc3ccccc3s2)nnc2c1CCCN2c1ccc(-c2cnn(CC34CC5(C)CC(C)(C3)CC(OCCN(C)CC(=O)O)(C5)C4)c2C)c(C(=O)O)n1]

Using the Amine Substitution and Hydrolysis General procedure III starting from Preparation 16 and methyl 2-(methylamino)acetate, hydrogen chloride (1:1) as the appropriate amine, the desired product was obtained. HRMS-ESI (m/z): [M+H]⁺ calculated for C₄₃H₅₂N₉O₅S: 806.3812, found: 806.3807.

### Preparation of P49: 6-[3-(1,3-benzothiazol-2-ylamino)-4-methyl-6,7-dihydro-5H-pyrido[2,3-c]pyridazin-8-yl]-3-[1-[[3-[3-[2-hydroxyethyl(methyl)amino]propyl]-5,7-dimethyl-1-adamantyl]methyl]-5-methyl-pyrazol-4-yl]pyridine-2-carboxylic acid

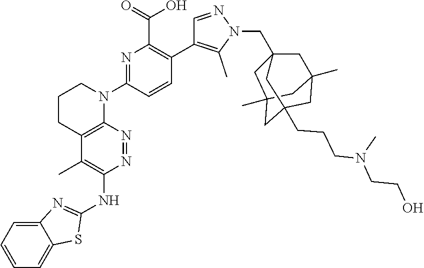 [SMILES: Cc1c(Nc2nc3ccccc3s2)nnc2c1CCCN2c1ccc(-c2cnn(CC34CC5(C)CC(C)(CC(CCCN(C)CCO)(C5)C3)C4)c2C)c(C(=O)O)n1]

Using the Amine substitution and Hydrolysis General procedure I starting from Preparation 13 and 2-(methylamino) ethanol as the appropriate amine, the desired product was obtained. HRMS-ESI (m/z): [M+H]⁺ calculated for C₄₄H₅₆N₉O₃S: 790.4227, found: 790.4227.

### Preparation of P50: 6-[3-(1,3-benzothiazol-2-ylamino)-4-methyl-6,7-dihydro-5H-pyrido[2,3-c]pyridazin-8-yl]-3-[1-[[3-[3-[3-methoxypropyl(methyl)amino]propyl]-5,7-dimethyl-1-adamantyl]methyl]-5-methyl-pyrazol-4-yl]pyridine-2-carboxylic acid

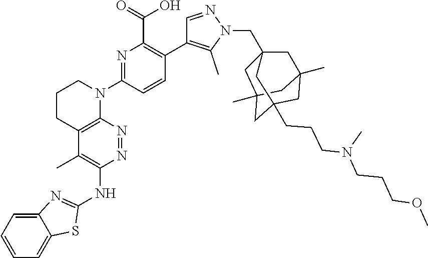 [SMILES: COCCCN(C)CCCC12CC3(C)CC(C)(C1)CC(Cn1ncc(-c4ccc(N5CCCc6c5nnc(Nc5nc7ccccc7s5)c6C)nc4C(=O)O)c1C)(C3)C2]

Using the Amine substitution and Hydrolysis General procedure I, starting from Preparation 13 and 3-methoxy-N-methyl-propan-1-amine as the appropriate amine, the desired product was obtained. HRMS-ESI (m/z): [M+H]⁺ calculated for C₄₆H₆₀N₉O₃S: 818.4540, found: 818.4537.

### Preparation of P51: 6-[3-(1,3-benzothiazol-2-ylamino)-4-methyl-6,7-dihydro-5H-pyrido[2,3-c]pyridazin-8-yl]-3-[1-[[3-[3-(3-methoxypropylamino)propyl]-5,7-dimethyl-1-adamantyl]methyl]-5-methyl-pyrazol-4-yl]pyridine-2-carboxylic acid

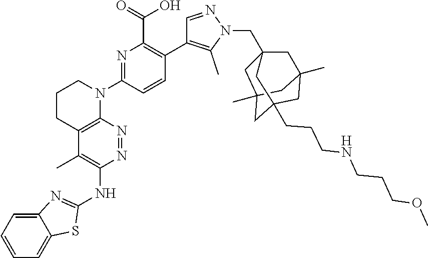 [SMILES: COCCCNCCCC12CC3(C)CC(C)(C1)CC(Cn1ncc(-c4ccc(N5CCCc6c5nnc(Nc5nc7ccccc7s5)c6C)nc4C(=O)O)c1C)(C3)C2]

Using the Amine substitution and Hydrolysis General procedure I starting from Preparation 13 and 3-methoxypropan-1-amine as the appropriate amine, the desired product was obtained. HRMS-ESI (m/z): [M+H]⁺ calculated for C₄₅H₅₈N₉O₃S: 804.4383, found: 804.4380.

### Preparation of P52: 3-[1-[[3-[3-(azepan-1-yl)propyl]-5,7-dimethyl-1-adamantyl]methyl]-5-methyl-pyrazol-4-yl]-6-[3-(1,3-benzothiazol-2-ylamino)-4-methyl-6,7-dihydro-5H-pyrido[2,3-c]pyridazin-8-yl]pyridine-2-carboxylic acid

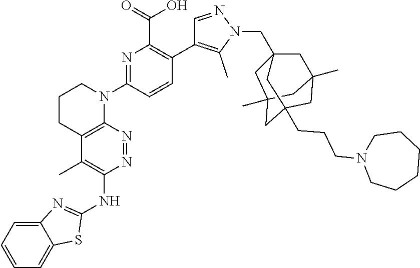 [SMILES: Cc1c(Nc2nc3ccccc3s2)nnc2c1CCCN2c1ccc(-c2cnn(CC34CC5(C)CC(C)(CC(CCCN6CCCCCC6)(C5)C3)C4)c2C)c(C(=O)O)n1]

Using the Amine substitution and Hydrolysis General procedure I starting from Preparation 13 and azepane as the appropriate amine, the desired product was obtained. HRMS-ESI (m/z): [M+H]⁺ calculated for C₄₇H₆₀N₉O₂S: 814.4591, found: 814.4588.

### Preparation of P53: 6-[3-(1,3-benzothiazol-2-ylamino)-4-methyl-6,7-dihydro-5H-pyrido[2,3-c]pyridazin-8-yl]-3-[1-[[3-[3-(carboxymethylamino)propyl]-5,7-dimethyl-1-adamantyl]methyl]-5-methyl-pyrazol-4-yl]pyridine-2-carboxylic acid

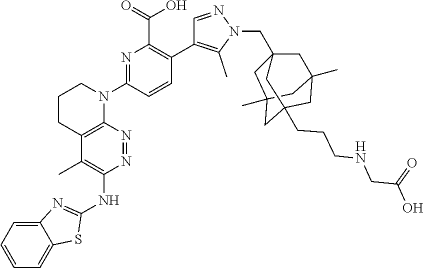 [SMILES: Cc1c(Nc2nc3ccccc3s2)nnc2c1CCCN2c1ccc(-c2cnn(CC34CC5(C)CC(C)(CC(CCCNCC(=O)O)(C5)C3)C4)c2C)c(C(=O)O)n1]

Using the Amine Substitution and Hydrolysis General procedure III starting from Preparation 13 and methyl 2-aminoacetate, hydrogen chloride (1:1) as the appropriate amine, the desired product was obtained. HRMS-ESI (m/z): [M+H]⁺ calculated for C₄₃H₅₂N₉O₄S: 790.3863, found: 790.3855.

### Preparation of P54: 6-[3-(1,3-benzothiazol-2-ylamino)-4-methyl-6,7-dihydro-5H-pyrido[2,3-c]pyridazin-8-yl]-3-[1-[[3-[3-(2-carboxyethylamino)propyl]-5,7-dimethyl-1-adamantyl]methyl]-5-methyl-pyrazol-4-yl]pyridine-2-carboxylic acid

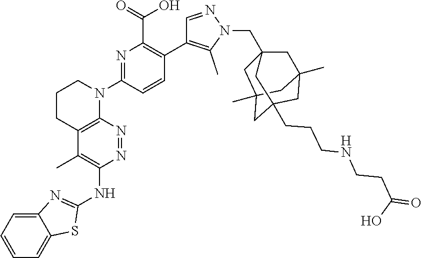 [SMILES: Cc1c(Nc2nc3ccccc3s2)nnc2c1CCCN2c1ccc(-c2cnn(CC34CC5(C)CC(C)(CC(CCCNCCC(=O)O)(C5)C3)C4)c2C)c(C(=O)O)n1]

Using the Amine Substitution and Hydrolysis General procedure III starting from Preparation 13 and methyl 3-aminopropanoate as the appropriate amine, the desired product was obtained. HRMS-ESI (m/z): [M+H]⁺ calculated for C₄₄H₅₄N₉O₄S: 804.4019, found: 804.4015.

### Preparation of P55: 6-[3-(1,3-benzothiazol-2-ylamino)-4-methyl-6,7-dihydro-5H-pyrido[2,3-c]pyridazin-8-yl]-3-[1-[[3-[3-[carboxymethyl(methyl)amino]propyl]-5,7-dimethyl-1-adamantyl]methyl]-5-methyl-pyrazol-4-yl]pyridine-2-carboxylic acid

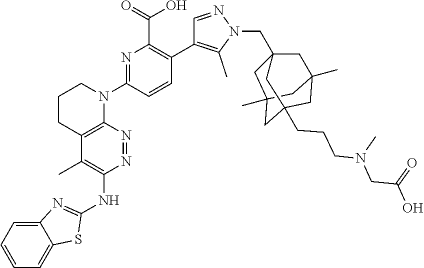 [SMILES: Cc1c(Nc2nc3ccccc3s2)nnc2c1CCCN2c1ccc(-c2cnn(CC34CC5(C)CC(C)(CC(CCCN(C)CC(=O)O)(C5)C3)C4)c2C)c(C(=O)O)n1]

Using the Amine Substitution and Hydrolysis General procedure III starting from Preparation 13 and methyl 2-(methylamino)acetate, hydrogen chloride (1:1) as the appropriate amine, the desired product was obtained. HRMS-ESI (m/z): [M+H]⁺ calculated for C₄₄H₅₄N₉O₄S: 804.4019, found: 804.4014.

### Preparation of P56: 6-[3-(1,3-benzothiazol-2-ylamino)-4-methyl-6,7-dihydro-5H-pyrido[2,3-c]pyridazin-8-yl]-3-[1-[[3-[3-[2-carboxyethyl(methyl)amino]propyl]-5,7-dimethyl-1-adamantyl]methyl]-5-methyl-pyrazol-4-yl]pyridine-2-carboxylic acid

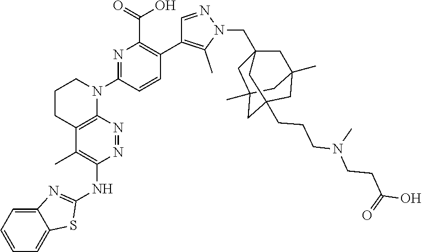 [SMILES: Cc1c(Nc2nc3ccccc3s2)nnc2c1CCCN2c1ccc(-c2cnn(CC34CC5(C)CC(C)(CC(CCCN(C)CCC(=O)O)(C5)C3)C4)c2C)c(C(=O)O)n1]

Using the Amine Substitution and Hydrolysis General procedure III starting from Preparation 13 and ethyl 3-(methylamino)propanoate, hydrogen chloride (1:1) as the appropriate amine, the desired product was obtained. HRMS-ESI (m/z): [M+H]⁺ calculated for C₄₅H₅₆N₉O₄S: 818.4176, found: 818.4167.

### Preparation of P57: 6-[3-(1,3-benzothiazol-2-ylamino)-4-methyl-6,7-dihydro-5H-pyrido[2,3-c]pyridazin-8-yl]-3-[1-[[3-[2-[3-carboxypropyl(methyl)amino]ethoxy]-5,7-dimethyl-1-adamantyl]methyl]-5-methyl-pyrazol-4-yl]pyridine-2-carboxylic acid

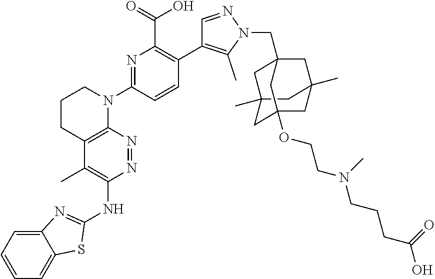 [SMILES: Cc1c(Nc2nc3ccccc3s2)nnc2c1CCCN2c1ccc(-c2cnn(CC34CC5(C)CC(C)(C3)CC(OCCN(C)CCCC(=O)O)(C5)C4)c2C)c(C(=O)O)n1]

Using the Amine Substitution and Hydrolysis General procedure III starting from Preparation 16 and methyl 4-(methylamino) butanoate, hydrogen chloride (1:1) as the appropriate amine, the desired product was obtained. HRMS-ESI (m/z): [M+H]⁺ calculated for C₄₅H₅₆N₉O₅S: 834.4125, found: 834.4115.

### Preparation of P58: 6-[3-(1,3-benzothiazol-2-ylamino)-4-methyl-6,7-dihydro-5H-pyrido[2,3-c]pyridazin-8-yl]-3-[1-[[3-[3-[3-carboxypropyl(methyl)amino]propyl]-5,7-dimethyl-1-adamantyl]methyl]-5-methyl-pyrazol-4-yl]pyridine-2-carboxylic acid

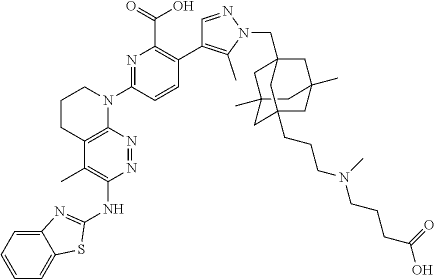 [SMILES: Cc1c(Nc2nc3ccccc3s2)nnc2c1CCCN2c1ccc(-c2cnn(CC34CC5(C)CC(C)(CC(CCCN(C)CCCC(=O)O)(C5)C3)C4)c2C)c(C(=O)O)n1]

Using the Amine Substitution and Hydrolysis General procedure III starting from Preparation 13 and methyl 4-(methylamino) butanoate, hydrogen chloride (1:1) as the appropriate amine, the desired product was obtained. HRMS-ESI (m/z): [M+H]⁺ calculated for C₄₆H₅₈N₉O₄S: 832.4332, found: 832.4324.

### Preparation of P59: 2-[3-(1,3-benzothiazol-2-ylamino)-4-methyl-6,7-dihydro-5H-pyrido[2,3-c]pyridazin-8-yl]-5-[3-[2-fluoro-4-[3-(3-hydroxypropylamino)prop-1-ynyl]phenoxy]propyl]thiazole-4-carboxylic acid

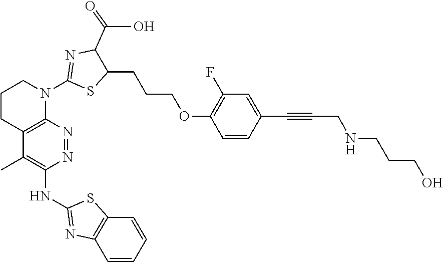 [SMILES: Cc1c(Nc2nc3ccccc3s2)nnc2c1CCCN2C1=NC(C(=O)O)C(CCCOc2ccc(C#CCNCCCO)cc2F)S1]

### Step A: 3-[tert-butyl(dimethyl)silyl]oxy-N-prop-2-ynyl-propan-1-amine

The mixture of 0.70 mL (3.0 mmol) of 3-bromopropoxy-tert-butyl-dimethyl-silane, 1.9 mL (10 eq) of propargylic amine and 1.6 mL (3 eq) of DIPEA in acetonitrile (15 mL) was stirred at 50° C. until no further conversion was observed. The reaction mixture was concentrated, diluted with DCM, and extracted with saturated NaHCO₃ and brine. The combined organic layers were dried and concentrated to give the desired product in quantitative yield. ¹H NMR (500 MHz, dmso-d₆) δ ppm 3.62 (t, 2H), 3.27 (d, 2H), 3.02 (t, 1H), 2.59 (t, 2H), 2.19 (brs, 1H), 1.57 (m, 2H), 0.86 (s, 9H), 0.02 (s, 6H); ¹³C NMR (500 MHz, dmso-d₆) δ ppm 73.9, 61.5, 45.2, 37.9, 32.7, 26.3, −4.8; HRMS (EI)(m/z): [M−CH₃]+ calculated for C₁₁H₂₂NOSi: 212.1471, found: 212.1467.

### Step B: ethyl 5-[3-[4-[3-[3-[tert-butyl(dimethyl)silyl]oxypropylamino]prop-1-ynyl]-2-fluoro-phenoxy]propyl]-2-(3-chloro-4-methyl-6,7-dihydro-5H-pyrido[2,3-c]pyridazin-8-yl)thiazole-4-carboxylate

Using Sonogashira General Procedure starting from 1.0 g (1.64 mmol) of the product of Preparation 15 and 737 mg (2 eq.) of the product from Step A as the appropriate acetylene, 1.16 g (96%) of the desired product was obtained. ¹H NMR (500 MHz, dmso-d₆) δ ppm 45.2 (t, 2H), 7.24 (dd, 1H), 7.17 (dd, 1H), 7.14 (t, 1H), 4.27 (br., 2H), 4.25 (q, 2H), 4.12 (t, 2H), 3.65 (t, 2H), 3.6 (s, 2H), 3.25 (t, 2H), 2.89 (t, 2H), 2.32 (s, 3H), 2.11 (m, 2H), 2.04 (m, 2H), 1.63 (m, 2H), 1.28 (t, 3H), 0.84 (s, 9H), 0.02 (s, 6H); ¹³C NMR (500 MHz, dmso-d₆) δ ppm 128.8, 119.1, 115.4, 68.3, 61.3, 60.7, 46.3, 45.2, 38.4, 32.4, 30.8, 26.3, 24.2, 23.1, 19.7, 15.7, 14.6, −4.8; HRMS-ESI (m/z): [M+H]⁺ calculated for C₃₅H₄₈ClFN₅O₄SSi: 716.2869, found: 716.2868.

### Step C: ethyl 2-[3-(1,3-benzothiazol-2-ylamino)-4-methyl-6,7-dihydro-5H-pyrido[2,3-c]pyridazin-8-yl]-5-[3-[4-[3-[3-[tert-butyl(dimethyl)silyl]oxypropylamino]prop-1-ynyl]-2-fluoro-phenoxy]propyl]thiazole-4-carboxylate

Using Buchwald General Procedure I starting from 1.16 g (1.57 mmol) of the product from Step B and 730 mg (2 eq) of 1,3-benzothiazol-2-amine, 598 mg (45%) of the desired product was obtained. ¹H NMR (500 MHz, dmso-d₆) δ ppm 7.87 (d, 1H), 7.49 (d, 1H), 7.37 (td, 1H), 7.25 (dd, 1H), 7.19 (t, 1H), 7.17 (t, 1H), 7.17 (m, 1H), 4.26 (br., 2H), 4.25 (q, 2H), 4.14 (t, 2H), 3.63 (t, 2H), 3.57 (s, 2H), 3.27 (t, 2H), 2.87 (t, 2H), 2.69 (t, 2H), 2.34 (s, 3H), 2.13 (m, 2H), 2.04 (m, 2H), 1.61 (m, 2H), 1.28 (t, 3H), 0.84 (s, 9H), 0.02 (s, 6H); ¹³C NMR (500 MHz, dmso-d₆) δ ppm 128.9, 126.5, 122.5, 122.3, 119.1, 116.3, 115.5, 68.4, 61.3, 60.6, 46.3, 45.2, 38.4, 32.4, 31.1, 26.3, 23.9, 23.2, 20.3, 14.6, 12.9, −4.9; HRMS-ESI (m/z): [M+H]⁺ calculated for C₄₂H₅₃FN₇O₄S₂Si: 830.3354, found: 830.3347.

### Step D: 2-[3-(1,3-benzothiazol-2-ylamino)-4-methyl-6,7-dihydro-5H-pyrido[2,3-c]pyridazin-8-yl]-5-[3-[2-fluoro-4-[3-(3-hydroxypropylamino)prop-1-ynyl]phenoxy]propyl]thiazole-4-carboxylic acid

The mixture of 590 mg (0.71 mmol) of the product from Step C and 298 mg of LiOH×H₂O (10 eq) in 7 mL of THF/water (1:1) was stirred at 60° C. until no further conversion was observed. The reaction mixture was treated with 0.71 mL (12 eq) of concentrated hydrogen chloride at 0° C. (pH=2-3) and stirred until no further conversion was observed. After the reaction mixture was concentrated to remove THF and lyophilization, the solid was dissolved in a 6N NH₃ solution in MeOH and purified by reverse phase chromatography (using 25 mM NH₄HCO₃ and MeCN as eluents) to give 100 mg (21%) of the desired product. HRMS-ESI (m/z): [M+H]⁺ calculated for C₃₄H₃₅FN₇O₄S₂: 688.2176, found: 688.2179.

### Preparation of P60: 6-[3-(1,3-benzothiazol-2-ylamino)-4-methyl-6,7-dihydro-5H-pyrido[2,3-c]pyridazin-8-yl]-3-[1-[[3-[2-[[(3S)-3,4-dihydroxybutyl]amino]ethoxy]-1-adamantyl]methyl]-5-methyl-pyrazol-4-yl]pyridine-2-carboxylic acid

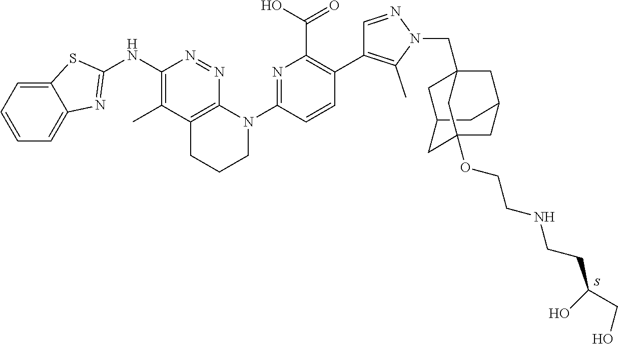

To the product from the Preparation 18 (0.066 mmol) in acetonitrile (30 ml/mmol) was added 2-[(4S)-2,2-dimethyl-1,3-dioxolan-4-yl]ethanamine, hydrogen chloride (1:1)(3 eq) and the reaction mixture was stirred at 60° C. for 48 h. After the addition of KOH solution (5 eq), the reaction mixture was stirred at 60° C. for 1 h. After the addition of HCl solution (10 eq), the reaction mixture was stirred at 60° C. for 1 h. The product was purified by preparative HPLC chromatography (using acetonitrile and 5 mM aqueous NH₄HCO₃ solution as eluents) to give the desired product. HRMS-ESI (m/z): [M+H]⁺ calculated for C₄₂H₅₂N₉O₅S: 794.3812, found: 794.3807.

### Preparation of P61: 6-[3-(1,3-benzothiazol-2-ylamino)-4-methyl-6,7-dihydro-5H-pyrido[2,3-c]pyridazin-8-yl]-3-[5-methyl-1-[[3-(2-pyrrolidin-1-ylethoxy)-1-adamantyl]methyl]pyrazol-4-yl]pyridine-2-carboxylic acid

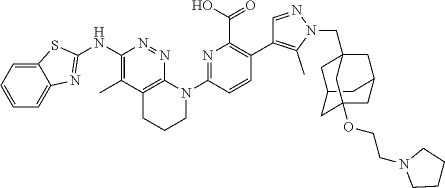 [SMILES: Cc1c(Nc2nc3ccccc3s2)nnc2c1CCCN2c1ccc(-c2cnn(CC34CC5CC(C3)CC(OCCN3CCCC3)(C5)C4)c2C)c(C(=O)O)n1]

Using the Amine substitution and Hydrolysis General procedure I, starting from Preparation 18 and pyrrolidine as the appropriate amine, the desired product was obtained. HRMS-ESI (m/z): [M+H]⁺ calculated for C₄₂H₅₀N₉O₃S: 760.3757, found: 760.3753.

### Preparation of P62: 6-[3-(1,3-benzothiazol-2-ylamino)-4-methyl-6,7-dihydro-5H-pyrido[2,3-c]pyridazin-8-yl]-3-[1-[[3-[2-[3-hydroxypropyl(methyl)amino]ethoxy]-5,7-dimethyl-1-adamantyl]methyl]-5-methyl-pyrazol-4-yl]pyridine-2-carboxylic acid

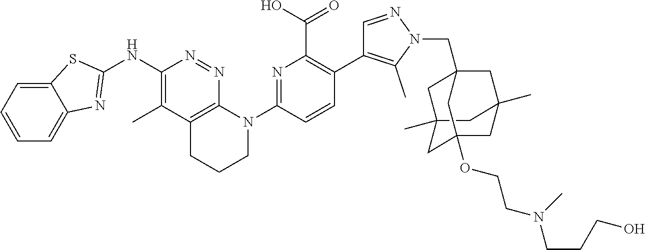 [SMILES: Cc1c(Nc2nc3ccccc3s2)nnc2c1CCCN2c1ccc(-c2cnn(CC34CC5(C)CC(C)(C3)CC(OCCN(C)CCCO)(C5)C4)c2C)c(C(=O)O)n1]

Using the Amine substitution and Hydrolysis General procedure I, starting from Preparation 16 and 3-(methylamino)propan-1-ol as the appropriate amine, the desired product was obtained. HRMS-ESI (m/z): [M+2H]2+ calculated for C₄₄H₅₆N₉O₄S: 403.7127, found: 403.7126.

### Preparation of P63: 3-[1-[[3,5-dimethyl-7-(2-pyrrolidin-1-ylethoxy)-1-adamantyl]methyl]-5-methyl-pyrazol-4-yl]-6-[3-[(5-fluoro-1,3-benzothiazol-2-yl)amino]-4-methyl-6,7-dihydro-5H-pyrido[2,3-c]pyridazin-8-yl]pyridine-2-carboxylic acid

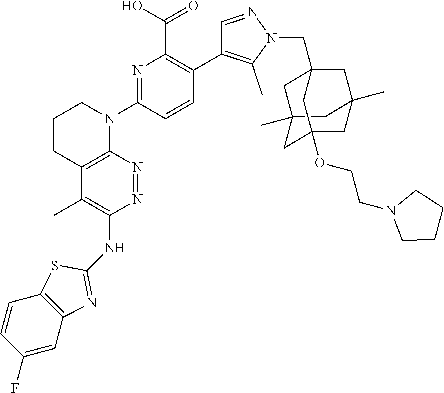 [SMILES: Cc1c(Nc2nc3cc(F)ccc3s2)nnc2c1CCCN2c1ccc(-c2cnn(CC34CC5(C)CC(C)(C3)CC(OCCN3CCCC3)(C5)C4)c2C)c(C(=O)O)n1]

### Step A: (4-methoxyphenyl)methyl 6-(3-chloro-4-methyl-6,7-dihydro-5H-pyrido[2,3-c]pyridazin-8-yl)-3-[1-[[3,5-dimethyl-7-[2-(p-tolylsulfonyloxy)ethoxy]-1-adamantyl]methyl]-5-methyl-pyrazol-4-yl]pyridine-2-carboxylate

To 260 mg (0.35 mmol) of Preparation 16, Step C in 2 mL of dichloromethane were added 0.5 mL (10 eq) of N,N-diethylethanamine and 457 mg (4 eq) of p-tolylsulfonyl 4-methylbenzenesulfonate, then the mixture was stirred for 0.5 h. The product was purified by column chromatography (silica gel, using DCM and EtOAc as eluents) to give 259 mg (85%) of the desired product. ¹H NMR (500 MHz, dmso-d₆) δ ppm 7.85 (d, 1H), 7.76 (d, 2H), 7.71 (d, 1H), 7.45 (d, 2H), 7.40 (s, 1H), 7.16 (d, 2H), 6.89 (d, 2H), 5.09 (s, 2H), 4.05 (t, 2H), 3.96 (t, 2H), 3.81 (s, 2H), 3.74 (s, 3H), 3.46 (t, 2H), 2.87 (t, 2H), 2.40 (s, 3H), 2.29 (s, 3H), 2.08 (s, 3H), 1.98 (qn, 2H), 1.29 (s, 2H), 1.13/1.11 (d+d, 4H), 1.11/1.06 (d+d, 4H), 0.98/0.90 (d+d, 2H), 0.81 (s, 6H); ¹³C NMR (500 MHz, dmso-d₆) δ ppm 140.1, 137.7, 130.6, 130.2, 128.2, 120.5, 114.3, 71.4, 66.8, 58.9, 58.4, 55.6, 49.8, 46.5, 46.0, 45.8, 42.9, 30.0, 24.6, 21.6, 21.0, 15.5, 10.8; HRMS-ESI (m/z): [M+H]⁺ calculated for C₄₈H₅₆ClN₆O₇S: 895.3620, found: 895.3619.

### Step B: (4-methoxyphenyl)methyl 6-(3-chloro-4-methyl-6,7-dihydro-5H-pyrido[2,3-c]pyridazin-8-yl)-3-[1-[[3,5-dimethyl-7-(2-pyrrolidin-1-ylethoxy)-1-adamantyl]methyl]-5-methyl-pyrazol-4-yl]pyridine-2-carboxylate

To 259 mg (0.29 mmol) of the product from Step A in 3 mL acetonitrile was added pyrrolidine (3 eq), and the reaction mixture was stirred at 55° C. for 18 h. The product was purified by column chromatography (silica gel, using DCM and MeOH as eluents) to give 221 mg (98%) of the desired product. ¹H NMR (500 MHz, dmso-d₆) δ ppm 7.85 (d, 1H), 7.70 (d, 1H), 7.40 (s, 1H), 7.18 (m, 2H), 6.91 (m, 2H), 5.10 (s, 2H), 3.96 (m, 2H), 3.86 (s, 2H), 3.75 (s, 3H), 3.60-2.90 (brs, 6H), 3.59 (brt, 2H), 2.87 (t, 2H), 2.29 (s, 3H), 2.11 (s, 3H), 2.10-1.70 (brs, 4H), 1.98 (m, 2H), 1.48-0.94 (m, 12H), 0.86 (s, 6H); ¹³C NMR (500 MHz, dmso-d₆) δ ppm 140.1, 137.7, 130.2, 120.5, 114.3, 66.8, 58.9, 56.9, 55.6, 46.0, 30.0, 24.6, 21.0, 15.5, 10.9; HRMS-ESI (m/z): [M+H]⁺ calculated for C₄₅H₅₇ClN₇O₄: 794.4161, found: 794.4160.

### Step C: 3-[1-[[3,5-dimethyl-7-(2-pyrrolidin-1-ylethoxy)-1-adamantyl]methyl]-5-methyl-pyrazol-4-yl]-6-[3-[(5-fluoro-1,3-benzothiazol-2-yl)amino]-4-methyl-6,7-dihydro-5H-pyrido[2,3-c]pyridazin-8-yl]pyridine-2-carboxylic acid

The mixture of 0.22 g (0.28 mmol) of the product from Step B, 93.5 mg (2 eq) of 5-fluoro-1,3-benzothiazol-2-amine, 25 mg (0.1 eq) of Pd₂(dba)₃, 32 mg (0.2 eq) of XantPhos, and 0.14 mL (3 eq) of DIPEA in 2 mL of butan-2-ol was kept at 100° C. in a microwave reactor for 1 h. The product was purified by column chromatography (using DCM/MeOH as eluents) to give the coupled product, which was treated with 3 eq of KOH in 2 mL of acetonitrile at 50° C. for 18 h. The hydrolysed product was purified by preparative HPLC chromatography (using acetonitrile and 5 mM aqueous NH₄HCO₃ solution as eluents) to give the desired product. HRMS-ESI (m/z): [M+H]⁺ calculated for C₄₄H₅₃FN₉O₃S: 806.3976, found: 806.3971.

### Preparation of P64: 3-[1-[[3,5-dimethyl-7-(2-pyrrolidin-1-ylethoxy)-1-adamantyl]methyl]-5-methyl-pyrazol-4-yl]-6-[4-methyl-3-[(6-methyl-1,3-benzothiazol-2-yl)amino]-6,7-dihydro-5H-pyrido[2,3-c]pyridazin-8-yl]pyridine-2-carboxylic acid

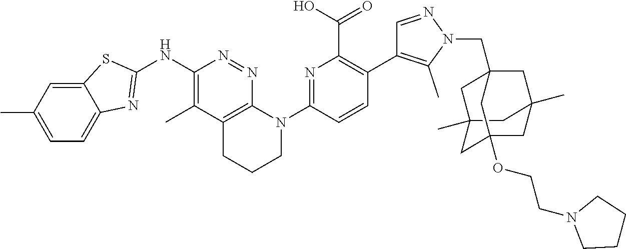 [SMILES: Cc1ccc2nc(Nc3nnc4c(c3C)CCCN4c3ccc(-c4cnn(CC56CC7(C)CC(C)(C5)CC(OCCN5CCCC5)(C7)C6)c4C)c(C(=O)O)n3)sc2c1]

### Step A: (4-methoxyphenyl)methyl 3-[1-[[3-(2-hydroxyethoxy)-5,7-dimethyl-1-adamantyl]methyl]-5-methyl-pyrazol-4-yl]-6-[4-methyl-3-[(6-methyl-1,3-benzothiazol-2-yl)amino]-6,7-dihydro-5H-pyrido[2,3-c]pyridazin-8-yl]pyridine-2-carboxylate

The mixture of 250 mg (0.34 mmol) of Preparation 16, Step C, 112 mg (2 eq) of 6-methyl-1,3-benzothiazol-2-amine, 31 mg (0.1 eq) of Pd₂(dba)₃, 39 mg (0.2 eq) of XantPhos, and 0.17 mL (3 eq) of DIPEA in 2.5 mL of cyclohexanol was kept at 130° C. for 2 h. The product was purified by column chromatography (using DCM/MeOH as eluents) to give 206 mg (71%) of the desired product. ¹H NMR (300 MHz, dmso-d₆) δ ppm 7.93 (d, 1H), 7.69 (d, 1H), 7.62 (brs, 1H), 7.45 (brs, 1H), 7.39 (s, 1H), 7.19 (m, 2H), 7.16 (brd, 1H), 6.91 (m, 2H), 5.10 (s, 2H), 4.45 (brs, 1H), 3.99 (m, 2H), 3.85 (s, 2H), 3.75 (s, 3H), 3.40 (t, 2H), 3.34 (t, 2H), 2.85 (t, 2H), 2.37 (s, 3H), 2.31 (s, 3H), 2.11 (s, 3H), 1.98 (m, 2H), 1.43-0.9 (m, 12H), 0.84 (s, 6H); ¹³C NMR (300 MHz, dmso-d₆) δ ppm 140.0, 137.6, 130.2, 127.5, 121.7, 118.9, 114.3, 66.7, 62.1, 61.5, 59.0, 55.6, 45.4, 30.1, 24.2, 21.7, 21.4, 12.6, 10.9; HRMS-ESI (m/z): [M+H]⁺ calculated for C₄₉H₅₇N₈O₅S: 869.4173, found: 869.4167.

### Step B: (4-methoxyphenyl)methyl 3-[1-[[3,5-dimethyl-7-[2-(p-tolylsulfonyloxy)ethoxy]-1-adamantyl]methyl]-5-methyl-pyrazol-4-yl]-6-[4-methyl-3-[(6-methyl-1,3-benzothiazol-2-yl)amino]-6,7-dihydro-5H-pyrido[2,3-c]pyridazin-8-yl]pyridine-2-carboxylate

To 203 mg (0.23 mmol) of the product from Step A in 2 mL of dichloromethane was added 0.16 mL (5 eq) of N,N-diethylethanamine and 150 mg (2 eq) of p-tolylsulfonyl 4-methylbenzenesulfonate, then the mixture was stirred for 18 h. The product was purified by column chromatography (silica gel, using DCM and EtOAc as eluents) to give 84 mg (38%) of the desired product. ¹H NMR (500 MHz, dmso-d₆) δ ppm 10.74 (br., 1H), 7.94 (d, 1H), 7.76 (dm, 2H), 7.69 (d, 1H), 7.61 (br., 1H), 7.45 (dm, 2H), 7.44 (br., 1H), 7.40 (s, 1H), 7.18 (dm, 2H), 7.17 (brd., 1H), 6.90 (dm, 2H), 5.09 (s, 2H), 4.05 (t, 2H), 3.99 (t, 2H), 3.82 (s, 2H), 3.74 (s, 3H), 3.47 (t, 2H), 2.84 (t, 2H), 2.40 (s, 3H), 2.37 (brs., 3H), 2.31 (s, 3H), 2.10 (s, 3H), 1.98 (m, 2H), 1.35-0.87 (m, 12H), 0.81 (s, 6H); ¹³C NMR (500 MHz, dmso-d₆) δ ppm 140.0, 137.7, 130.6, 130.1, 128.1, 127.5, 121.8, 118.9, 114.3, 71.5, 66.7, 58.9, 58.4, 55.6, 45.4, 30.0, 24.3, 21.6, 21.6, 21.4, 12.5, 10.9; HRMS-ESI (m/z): [M+H]⁺ calculated for C₅₆H₆₃N₈O₇S₂: 1023.4261, found: 1023.4265.

### Step C: 3-[1-[[3,5-dimethyl-7-(2-pyrrolidin-1-ylethoxy)-1-adamantyl]methyl]-5-methyl-pyrazol-4-yl]-6-[4-methyl-3-[(6-methyl-1,3-benzothiazol-2-yl)amino]-6,7-dihydro-5H-pyrido[2,3-c]pyridazin-8-yl]pyridine-2-carboxylic acid

To 84 mg (0.082 mmol) of the product from Step B in 1 mL acetonitrile was added pyrrolidine (3 eq) and the reaction mixture was stirred at 55° C. for 18 h. After treatment with 5 eq of KOH, the mixture was stirred at 55° C. for 1 h and the product was purified by preparative HPLC chromatography (using acetonitrile and 5 mM aqueous NH₄HCO₃ solution as eluents) to give the desired product. HRMS-ESI (m/z): [M+H]⁺ calculated for C₄₅H₅₆N₉O₃S: 802.4227, found: 802.4227.

### Preparation of P65: 3-[1-[[3,5-dimethyl-7-(2-pyrrolidin-1-ylethoxy)-1-adamantyl]methyl]-5-methyl-pyrazol-4-yl]-6-[3-[(6-fluoro-1,3-benzothiazol-2-yl)amino]-4-methyl-6,7-dihydro-5H-pyrido[2,3-c]pyridazin-8-yl]pyridine-2-carboxylic acid

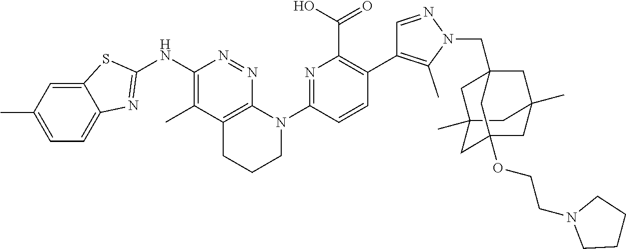 [SMILES: Cc1ccc2nc(Nc3nnc4c(c3C)CCCN4c3ccc(-c4cnn(CC56CC7(C)CC(C)(C5)CC(OCCN5CCCC5)(C7)C6)c4C)c(C(=O)O)n3)sc2c1]

### Step A: (4-methoxyphenyl)methyl 6-[3-[(6-fluoro-1,3-benzothiazol-2-yl)amino]-4-methyl-6,7-dihydro-5H-pyrido[2,3-c]pyridazin-8-yl]-3-[1-[[3-(2-hydroxyethoxy)-5,7-dimethyl-1-adamantyl]methyl]-5-methyl-pyrazol-4-yl]pyridine-2-carboxylate

The mixture of 250 mg (0.34 mmol) of Preparation 16, Step C, 114 mg (2 eq) of 6-fluoro-1,3-benzothiazol-2-amine, 31 mg (0.1 eq) of Pd₂(dba)₃, 39 mg (0.2 eq) of XantPhos, and 0.17 mL (3 eq) of DIPEA in 2.5 mL of cyclohexanol was kept at 130° C. for 2 h. The product was purified by column chromatography (using DCM/MeOH as eluents) to give 158 mg (55%) of the desired product. ¹H NMR (500 MHz, dmso-d₆) δ ppm 10.87 (brs, 1H), 7.94 (d, 1H), 7.77 (brd, 1H), 7.69 (d, 1H), 7.57 (brs, 1H), 7.39 (s, 1H), 7.20 (m, 1H), 7.19 (m, 2H), 6.91 (m, 2H), 5.10 (s, 2H), 4.45 (brs, 1H), 3.99 (m, 2H), 3.85 (s, 2H), 3.75 (s, 3H), 3.40 (t, 2H), 3.34 (t, 2H), 2.85 (t, 2H), 2.31 (s, 3H), 2.11 (s, 3H), 1.98 (m, 2H), 1.43-0.91 (m, 12H), 0.84 (s, 6H); ¹³C NMR (500 MHz, dmso-d₆) δ ppm 140.0, 137.7, 130.2, 118.9, 114.3, 114.0, 108.4, 66.7, 62.1, 61.5, 59.0, 55.6, 45.4, 30.1, 24.3, 21.6, 12.5, 10.9; HRMS-ESI (m/z): [M+H]⁺ calculated for C₄₈H₅₄FN₈O₅S: 873.3922, found: 873.3917.

### Step B: (4-methoxyphenyl)methyl 3-[1-[[3,5-dimethyl-7-[2-(p-tolylsulfonyloxy)ethoxy]-1-adamantyl]methyl]-5-methyl-pyrazol-4-yl]-6-[3-[(6-fluoro-1,3-benzothiazol-2-yl)amino]-4-methyl-6,7-dihydro-5H-pyrido[2,3-c]pyridazin-8-yl]pyridine-2-carboxylate

To 158 mg (0.23 mmol) of the product from Step A in 2 mL of dichloromethane was added 0.125 mL (5 eq) of N,N-diethylethanamine and 117 mg (2 eq) of p-tolylsulfonyl 4-methylbenzenesulfonate, then the mixture was stirred for 18 h. The product was purified by column chromatography (silica gel, using DCM and EtOAc as eluents) to give 71 mg (41%) of the desired product. ¹H NMR (500 MHz, dmso-d₆) δ ppm 10.88 (brs, 1H), 7.94 (d, 1H), 7.77 (br., 1H), 7.76 (dm, 2H), 7.69 (d, 1H), 7.59 (br., 1H), 7.45 (dm, 2H), 7.40 (s, 1H), 7.21 (t, 1H), 7.17 (dm, 2H), 6.90 (dm, 2H), 5.09 (s, 2H), 4.05 (t, 2H), 4.00 (m, 2H), 3.82 (s, 2H), 3.74 (s, 3H), 3.47 (t, 2H), 2.85 (t, 2H), 2.40 (s, 3H), 2.32 (s, 3H), 2.10 (s, 3H), 1.98 (m, 2H), 1.35-0.87 (m, 12H), 0.81 (s, 6H); ¹³C NMR (500 MHz, dmso-d₆) δ ppm 140.0, 137.7, 130.6, 130.1, 128.1, 118.9, 114.3, 114.0, 108.4, 71.5, 66.7, 58.9, 58.4, 55.6, 45.4, 30.0, 24.3, 21.6, 21.6, 12.5, 10.9; HRMS-ESI (m/z): [M+H]⁺ calculated for C₅₅H₆₀FN₈O₇S₂: 1027.4010, found: 1027.4003.

### Step C: 3-[1-[[3,5-dimethyl-7-(2-pyrrolidin-1-ylethoxy)-1-adamantyl]methyl]-5-methyl-pyrazol-4-yl]-6-[3-[(6-fluoro-1,3-benzothiazol-2-yl)amino]-4-methyl-6,7-dihydro-5H-pyrido[2,3-c]pyridazin-8-yl]pyridine-2-carboxylic acid

To 71 mg (0.069 mmol) of the product from Step B in 1 mL acetonitrile was added pyrrolidine (3 eq) and the reaction mixture was stirred at 55° C. for 18 h. After treatment with 5 eq of KOH, the mixture was stirred at 55° C. for 1 h and the product was purified by preparative HPLC chromatography (using acetonitrile and 5 mM aqueous NH₄HCO₃ solution as eluents) to give the desired product. HRMS-ESI (m/z): [M+H]⁺ calculated for C₄₄H₅₃FN₉O₃S: 806.3976, found: 806.3969.

### Preparation of P66: 6-[3-(1,3-benzothiazol-2-ylamino)-4-methyl-6,7-dihydro-5H-pyrido[2,3-c]pyridazin-8-yl]-3-[1-[[3-[3-(dimethylamino)propyl]-5,7-dimethyl-1-adamantyl]methyl]-5-methyl-pyrazol-4-yl]pyridine-2-carboxylic acid

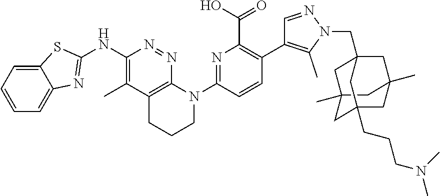 [SMILES: Cc1c(Nc2nc3ccccc3s2)nnc2c1CCCN2c1ccc(-c2cnn(CC34CC5(C)CC(C)(CC(CCCN(C)C)(C5)C3)C4)c2C)c(C(=O)O)n1]

Using the Amine substitution and Hydrolysis General procedure I, starting from Preparation 13 and N-methylmethanamine as the appropriate amine, the desired product was obtained. HRMS-ESI (m/z): [M+H]⁺ calculated for C₄₃H₅₄N₉O₂S: 760.4121, found: 760.4114.

### Preparation of P67: 6-[3-(1,3-benzothiazol-2-ylamino)-4-methyl-6,7-dihydro-5H-pyrido[2,3-c]pyridazin-8-yl]-3-[5-methyl-1-[[3-[2-(4-methylpiperazin-1-yl)ethoxy]-1-adamantyl]methyl]pyrazol-4-yl]pyridine-2-carboxylic acid

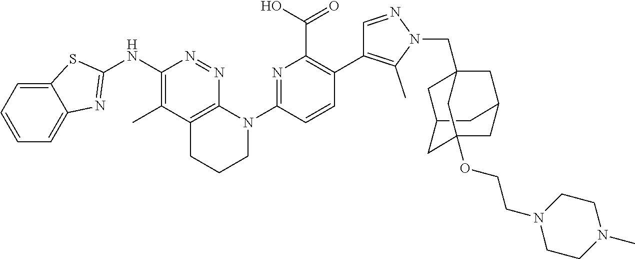 [SMILES: Cc1c(Nc2nc3ccccc3s2)nnc2c1CCCN2c1ccc(-c2cnn(CC34CC5CC(C3)CC(OCCN3CCN(C)CC3)(C5)C4)c2C)c(C(=O)O)n1]

Using the Amine substitution and Hydrolysis General procedure I, starting from Preparation 18 and 1-methylpiperazine as the appropriate amine, the desired product was obtained. HRMS-ESI (m/z): [M+H]⁺ calculated for C₄₃H₅₃N₁₀O₃S: 789.4022, found: 789.4014.

### Preparation of P68: 6-[3-(1,3-benzothiazol-2-ylamino)-4-methyl-6,7-dihydro-5H-pyrido[2,3-c]pyridazin-8-yl]-3-[1-[[3-[3-[3-hydroxypropyl(methyl)amino]propyl]-5,7-dimethyl-1-adamantyl]methyl]-5-methyl-pyrazol-4-yl]pyridine-2-carboxylic acid

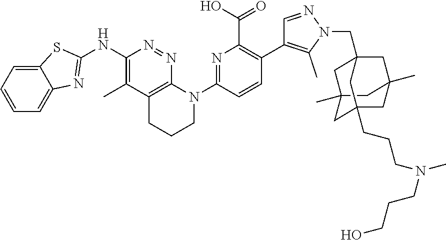 [SMILES: Cc1c(Nc2nc3ccccc3s2)nnc2c1CCCN2c1ccc(-c2cnn(CC34CC5(C)CC(C)(CC(CCCN(C)CCCO)(C5)C3)C4)c2C)c(C(=O)O)n1]

Using the Amine substitution and Hydrolysis General procedure I, starting from Preparation 13 and 3-(methylamino)propan-1-ol as the appropriate amine, the desired product was obtained. HRMS-ESI (m/z): [M+H]⁺ calculated for C₄₅H₅₈N₉O₃S: 804.4383, found: 804.4375.

### Preparation of P69: 6-[3-(1,3-benzothiazol-2-ylamino)-4-methyl-6,7-dihydro-5H-pyrido[2,3-c]pyridazin-8-yl]-3-[1-[[3-[3-[[(3S)-3,4-dihydroxybutyl]amino]propyl]-5,7-dimethyl-1-adamantyl]methyl]-5-methyl-pyrazol-4-yl]pyridine-2-carboxylic acid

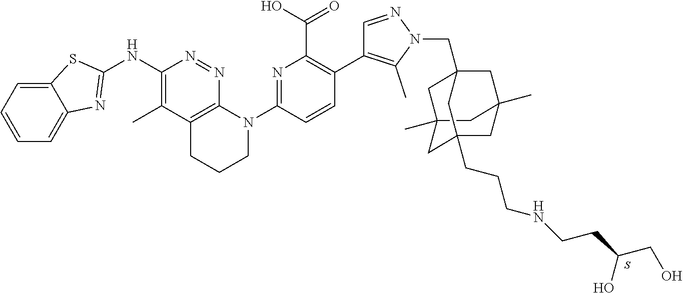

To the product from the Preparation 13 (0.074 mmol) in 2 mL of acetonitrile was added the 2-[(4S)-2,2-dimethyl-1,3-dioxolan-4-yl]ethanamine, hydrogen chloride (1:1)(4 eq) and the reaction mixture was stirred at 60° C. for 18 h. After the addition of KOH solution (5 eq), the reaction mixture was stirred at 60° C. for 1 h. After the addition of HCl solution (10 eq), the reaction mixture was stirred at 60° C. for 0.5 h. The product was purified by preparative HPLC chromatography (using acetonitrile and 5 mM aqueous NH₄HCO₃ solution as eluents) to give the desired product. HRMS-ESI (m/z): [M+H]⁺ calculated for C₄₅H₅₈N₉O₄S: 820.4332, found: 820.4323.

### Preparation of P70: 6-[3-(1,3-benzothiazol-2-ylamino)-4-methyl-6,7-dihydro-5H-pyrido[2,3-c]pyridazin-8-yl]-3-[1-[[3-[2-[2-carboxyethyl(methyl)amino]ethoxy]-5,7-dimethyl-1-adamantyl]methyl]-5-methyl-pyrazol-4-yl]pyridine-2-carboxylic acid

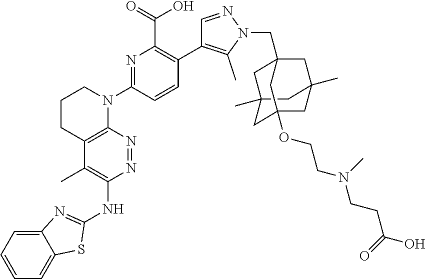 [SMILES: Cc1c(Nc2nc3ccccc3s2)nnc2c1CCCN2c1ccc(-c2cnn(CC34CC5(C)CC(C)(C3)CC(OCCN(C)CCC(=O)O)(C5)C4)c2C)c(C(=O)O)n1]

Using the Amine Substitution and Hydrolysis General procedure III, starting from Preparation 16 and ethyl 3-(methylamino)propanoate, hydrogen chloride (1:1) as the appropriate amine, the desired product was obtained. HRMS-ESI (m/z): [M+H]⁺ calculated for C₄₄H₅₄N₉O₅S: 820.3968, found: 820.3962.

### Preparation of P71: 6-[3-(1,3-benzothiazol-2-ylamino)-4-methyl-6,7-dihydro-5H-pyrido[2,3-c]pyridazin-8-yl]-3-[1-[[3-[2-(2-carboxyethylamino)ethoxy]-5,7-dimethyl-1-adamantyl]methyl]-5-methyl-pyrazol-4-yl]pyridine-2-carboxylic acid

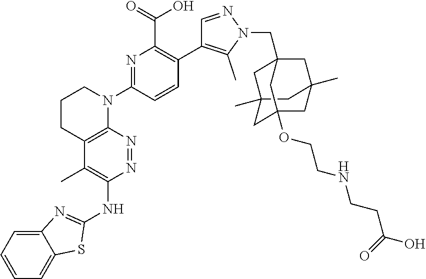 [SMILES: Cc1c(Nc2nc3ccccc3s2)nnc2c1CCCN2c1ccc(-c2cnn(CC34CC5(C)CC(C)(C3)CC(OCCNCCC(=O)O)(C5)C4)c2C)c(C(=O)O)n1]

Using the Amine Substitution and Hydrolysis General procedure III, starting from Preparation 16 and methyl 3-aminopropanoate as the appropriate amine, the desired product was obtained. HRMS-ESI (m/z): [M+H]⁺ calculated for C₄₃H₅₂N₉O₅S: 806.3812, found: 806.3793.

### Preparation of P72: 6-[3-(1,3-benzothiazol-2-ylamino)-4-methyl-6,7-dihydro-5H-pyrido[2,3-c]pyridazin-8-yl]-3-[1-[[3-[3-(4-hydroxybutylamino)propyl]-5,7-dimethyl-1-adamantyl]methyl]-5-methyl-pyrazol-4-yl]pyridine-2-carboxylic acid

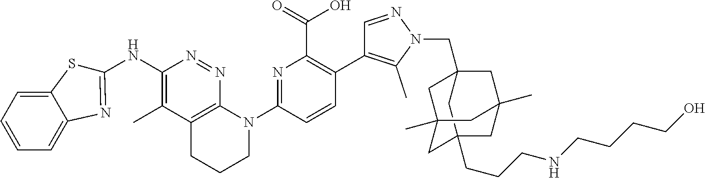 [SMILES: Cc1c(Nc2nc3ccccc3s2)nnc2c1CCCN2c1ccc(-c2cnn(CC34CC5(C)CC(C)(CC(CCCNCCCCO)(C5)C3)C4)c2C)c(C(=O)O)n1]

Using the Amine substitution and Hydrolysis General procedure I, starting from Preparation 13 and 4-aminobutan-1-ol as the appropriate amine, the desired product was obtained. HRMS-ESI (m/z): [M+H]⁺ calculated for C₄₅H₅₈N₉O₃S: 804.4383, found: 804.4383.

### Preparation of P73: [6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-2-pyridyl]methanol

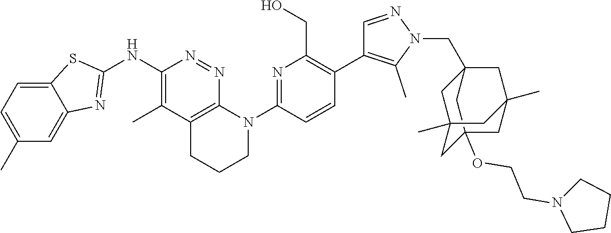 [SMILES: Cc1ccc2sc(Nc3nnc4c(c3C)CCCN4c3ccc(-c4cnn(CC56CC7(C)CC(C)(C5)CC(OCCN5CCCC5)(C7)C6)c4C)c(CO)n3)nc2c1]

### Step A: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]pyridine-2-carboxylate

Using the Amine substitution and Hydrolysis General procedure I without the hydrolysis step, starting from Preparation 16 and pyrrolidine as the appropriate amine, 190 mg of the desired product was obtained. ¹H NMR (500 MHz, dmso-d₆) δ ppm 7.95 (d, 1H), 7.81 (d, 1H), 7.68 (d, 1H), 7.50 (brd., 1H), 7.39 (s, 1H), 7.35 (t, 1H), 7.19 (dm, 2H), 7.16 (t, 1H), 6.91 (dm, 2H), 5.10 (s, 2H), 3.99 (t, 2H), 3.85 (s, 2H), 3.74 (s, 3H), 3.41 (t, 2H), 2.85 (t, 2H), 2.46 (t, 2H), 2.41 (br., 4H), 2.32 (s, 3H), 2.11 (s, 3H), 1.98 (m, 2H), 1.62 (m, 4H), 1.40 (s, 2H), 1.28/1.22 (d+d, 4H), 1.19/1.13 (d+d, 4H), 1.03/0.94 (d+d, 2H), 0.84 (s, 6H); ¹³C NMR (500 MHz, dmso-d₆) δ ppm 140.0, 137.7, 130.2, 126.4, 122.4, 122.1, 118.9, 114.3, 66.7, 59.5, 59.0, 56.6, 55.6, 54.5, 50.0, 46.9, 46.0, 45.4, 43.2, 30.1, 24.3, 23.6, 21.7, 12.6, 10.9; HRMS-ESI (m/z): [M+H]⁺ calculated for C₅₂H₆₂N₉O₄S: 908.4645, found: 908.4633.

### Step B: [6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-2-pyridyl]methanol

To 190 mg (0.21 mmol) of the product from Step A in 4.2 mL of tetrahydrofuran was added 24 mg (3 eq) of LiAlH₄, and the mixture was stirred for 40 min. After quenching with 0.1% TFA in MeOH and filtration, the product was purified via preparative HPLC (MeCN and 0.1% TFA solution as eluents) to give 110 mg (67%) of the desired product. HRMS-ESI (m/z): [M+H]⁺ calculated for C₄₄H₅₆N₉O₂S: 774.4277, found: 774.4269.

### Preparation of P74: [6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-2-pyridyl]-pyrrolidin-1-yl-methanone

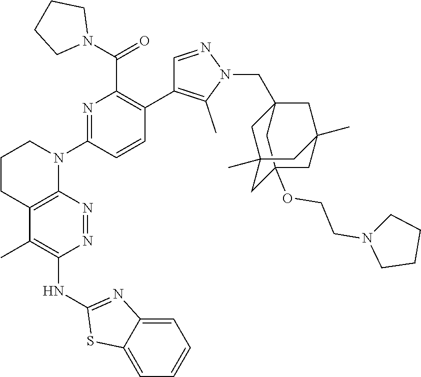 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)N2CCCC2)n1]

To 50 mg (0.063 mmol) of P21, 9.37 mg (2.1 eq) of pyrrolidine, and 0.032 mL (3 eq) of DIPEA in 0.5 mL of DMF were added 36 mg (1.5 eq) of HATU at 0° C., then the mixture was stirred for 18 h at room temperature. After pouring the reaction mixture into water, the precipitated solid was filtered out, washed with water, and dried. The product was purified by column chromatography (amino column, using DCM and MeOH as eluents) to give 29 mg (65%) of the desired product. HRMS-ESI (m/z): [M+H]⁺ calculated for C₄₈H₆₁N₁₀O₂S: 841.4699, found: 841.4698.

### Preparation of P75: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]-N-isopropyl-pyridine-2-carboxamide

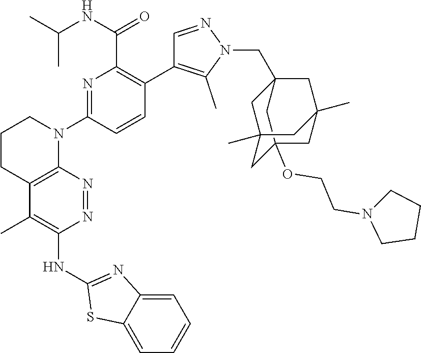 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)NC(C)C)n1]

To 50 mg (0.063 mmol) of P21, 9.37 mg (2 eq) of propan-2-amine, and 0.032 mL (3 eq) of DIPEA in 0.5 mL of DMF were added 36 mg (1.5 eq) of HATU at 0° C., then the mixture was stirred for 18 h at room temperature. After pouring the reaction mixture into water, the precipitated solid was filtered out, washed with water, and dried. The product was purified by column chromatography (amino column, using DCM and MeOH as eluents) to give 34 mg (76%) of the desired product. HRMS-ESI (m/z): [M+H]⁺ calculated for C₄₇H₆₁N₁₀O₂S: 829.4699, found: 829.4694.

### Preparation of P76: 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]pyridine-2-carboxamide

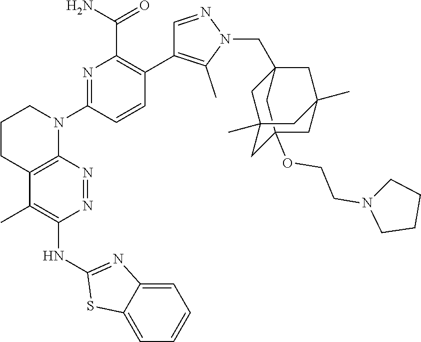 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(N)=O)n1]

To 50 mg (0.063 mmol) of P21 and 18 mg (1.3 eq) of tert-butoxycarbonyl tert-butyl carbonate in 0.5 mL of dioxane was added 0.006 mL of pyridine, then the mixture was stirred for 10 min. After treating the mixture with 6.5 mg (1.3 eq) of NH₄HCO₃, the reaction was stirred for 5 days. The product was purified by column chromatography (amino column, using DCM and MeOH as eluents) to give 17 mg (47%) of the desired product. HRMS-ESI (m/z): [M+H]⁺ calculated for C₄₄H₅₅N₁₀O₂S: 787.4230, found: 787.4226.

### Example 2. Synthesis and Characterization of Payload Precursors

“PMB-protected payload” is also referred to as a precursor of the considered payload for the purpose of the preparation of a Linker/Payload.

### Preparation A for Precursors: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-[2-(p-tolylsulfonyloxy)ethoxy]-1-adamantyl]methyl]-5-methyl-pyrazol-4-yl]pyridine-2-carboxylate

### Step A: (4-methoxyphenyl)methyl 3-[1-[[3-[2-[tert-butyl(diphenyl)silyl]oxyethoxy]-5,7-dimethyl-1-adamantyl]methyl]-5-methyl-pyrazol-4-yl]-6-[3-(3,6-dichloro-5-methyl-pyridazin-4-yl)propylamino]pyridine-2-carboxylate

The mixture of the product from Preparation 11 (9.78 g, 18.1 mmol), the product from Preparation 7 (13.6 g, 1.1 eq), Pd(AtaPhos)₂Cl₂ (801 mg, 0.1 eq), and Cs₂CO₃ (17.7 g, 3 eq) in 1,4-dioxane (109 mL) and H₂O (18 mL) was stirred at 80° C. for 8 h. After quenching the cooled reaction with brine, the mixture was extracted with EtOAc and the combined organic layers were dried and concentrated to give the desired product (21.9 g, 119%), which was used in the next step without further purification. ¹H NMR (400 MHz, DMSO-d₆): δ ppm 7.68-7.35 (m, 10H), 7.31 (d, 1H), 7.27 (s, 1H), 7.11 (dm, 2H), 6.98 (t, 1H), 6.83 (dm, 2H), 6.62 (d, 1H), 4.99 (s, 2H), 3.80 (s, 2H), 3.70 (s, 3H), 3.65 (t, 2H), 3.44 (t, 2H), 3.34 (q, 2H), 2.84 (m, 2H), 2.34 (s, 3H), 2.01 (s, 3H), 1.77 (m, 2H), 1.38-0.89 (m, 12H), 0.97 (s, 9H), 0.82 (s, 6H); ¹³C NMR (500 MHz, dmso-d₆) δ ppm 140.4, 137.6, 130.1, 114.2, 110.3, 66.3, 64.4, 61.7, 59.0, 55.5, 40.9, 30.1, 28.1, 27.3, 27.1, 16.4, 10.8; HRMS-ESI (m/z): [M+H]⁺ calculated for C₅₇H₆₉Cl₂N₆O₅Si: 1015.4475 found: 1015.4474.

### Step B: (4-methoxyphenyl)methyl 3-[1-[[3-[2-[tert-butyl(diphenyl)silyl]oxyethoxy]-5,7-dimethyl-1-adamantyl]methyl]-5-methyl-pyrazol-4-yl]-6-(3-chloro-4-methyl-6,7-dihydro-5H-pyrido[2,3-c]pyridazin-8-yl)pyridine-2-carboxylate

The mixture of the product from Step A (21.9 g, 21.6 mmol), Cs₂CO₃ (14 g, 2 eq), DIPEA (7.5 mL, 2 eq) and Pd(Ataphos)₂Cl₂ (954 mg, 0.1 eq) in 1,4-dioxane (108 mL) was stirred at 110° C. for 18 h. After quenching with water and extracting with EtOAc, the combined organic phases were dried, concentrated, and purified by column chromatography (silica gel, DCM and EtOAc as eluents) to give the desired product (8.4 g, 40%). ¹H NMR (400 MHz, DMSO-d₆): δ ppm 7.84 (d, 1H), 7.67 (d, 1H), 7.65 (d, 4H), 7.44 (t, 2H), 7.41 (s, 1H), 7.40 (t, 4H), 7.15 (d, 2H), 6.87 (d, 2H), 5.07 (s, 2H), 3.96 (t, 2H), 3.83 (s, 2H), 3.71 (s, 3H), 3.66 (t, 2H), 3.45 (t, 2H), 2.86 (t, 2H), 2.29 (s, 3H), 2.08 (s, 3H), 1.97 (qn, 2H), 1.38 (s, 2H), 1.25/1.18 (d+d, 4H), 1.18/1.12 (d+d, 4H), 1.01/0.93 (d+d, 2H), 0.97 (s, 9H), 0.82 (s, 6H); ¹³C NMR (100 MHz, DMSO-d₆) δ ppm 166.8, 159.7, 156.3, 153.6, 150.8, 147.7, 140.1, 137.6, 137.3, 136.0, 135.6, 133.8, 130.2, 130.2, 129.1, 128.2, 127.7, 123.0, 120.4, 115.6, 114.3, 74.2, 66.8, 64.4, 61.7, 59.3, 55.6, 49.9, 46.8, 46.0, 46.0, 43.3, 39.7, 33.6, 30.1, 27.1, 24.6, 21.0, 19.3, 15.5, 10.8; HRMS-ESI (m/z): [M+H]⁺ calculated for C₅₇H₆₈ClN₆O₅Si: 979.4709 found: 979.4710.

### Step C: (4-methoxyphenyl)methyl 6-(3-chloro-4-methyl-6,7-dihydro-5H-pyrido[2,3-c]pyridazin-8-yl)-3-[1-[[3-(2-hydroxyethoxy)-5,7-dimethyl-1-adamantyl]methyl]-5-methyl-pyrazol-4-yl]pyridine-2-carboxylate

To the product from Step B (8.4 g, 8.6 mmol) in THF (86 mL) was added a 1 M solution of TBAF in THF (9.4 mL, 1.1 eq) at 0° C. and the reaction mixture was stirred at room temperature for 1.5 h. After quenching with a saturated solution of NH₄Cl and extracted with EtOAc, the combined organic phases were washed with brine, dried, concentrated, and purified by column chromatography (silica gel, DCM and MeOH as eluents) to give the desired product (4.7 g, 74%). ¹H NMR (400 MHz, DMSO-d₆): δ ppm 7.85 (d, 1H), 7.70 (d, 1H), 7.39 (s, 1H), 7.18 (d, 2H), 6.90 (d, 2H), 5.10 (s, 2H), 4.45 (t, 1H), 3.96 (t, 2H), 3.84 (s, 2H), 3.74 (s, 3H), 3.40 (q, 2H), 3.33 (t, 2H), 2.86 (t, 2H), 2.29 (s, 3H), 2.09 (s, 3H), 1.98 (qn, 2H), 1.39 (s, 2H), 1.27/1.21 (d+d, 4H), 1.18/1.12 (d+d, 4H), 1.03/0.94 (d+d, 2H), 0.84 (s, 6H); ¹³C NMR (100 MHz, DMSO-d₆) δ ppm 166.8, 159.7, 156.3, 153.6, 150.8, 147.8, 140.2, 137.6, 137.3, 136.0, 130.2, 129.1, 127.7, 123.0, 120.4, 115.6, 114.3, 74.0, 66.8, 62.2, 61.5, 59.0, 55.6, 50.0, 46.9, 46.0, 46.0, 43.3, 39.7, 33.5, 30.1, 24.6, 21.0, 15.5, 10.9; HRMS-ESI (m/z): [M+H]⁺ calculated for C₄₁H₅₀ClN₆O₅: 741.3531 found: 741.3530.

### Step D: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-(2-hydroxyethoxy)-5,7-dimethyl-1-adamantyl]methyl]-5-methyl-pyrazol-4-yl]pyridine-2-carboxylate

The mixture of the product from Step C (4.7 g, 6.3 mmol), 1,3-benzothiazol-2-amine (1.9 g, 2 eq), Pd₂dba₃ (580 mg, 0.1 eq), XantPhos (730 mg, 0.2 eq), and DIPEA (3.3 mL, 3 eq) in cyclohexanol (38 mL) was stirred at 130° C. for 2 h. Purification by column chromatography (silica gel, heptane, EtOAc and MeCN as eluents) afforded the desired product (3.83 g, 71%). ¹H NMR (400 MHz, DMSO-d₆): δ ppm 7.95 (d, 1H), 7.81 (brd, 1H), 7.69 (d, 1H), 7.49 (brs, 1H), 7.39 (s, 1H), 7.35 (m, 1H), 7.19 (m, 2H), 7.16 (m, 1H), 6.91 (m, 2H), 5.10 (s, 2H), 4.46 (t, 1H), 3.99 (m, 2H), 3.85 (s, 2H), 3.75 (s, 3H), 3.40 (m, 2H), 3.34 (t, 2H), 2.85 (t, 2H), 2.32 (s, 3H), 2.11 (s, 3H), 1.99 (m, 2H), 1.45-0.9 (m, 12H), 0.84 (s, 6H); HRMS-ESI (m/z): [M+H]⁺ calculated for C₄₈H₅₅N₈O₅S: 855.4016 found: 855.4011.

### Step E: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-[2-(p-tolylsulfonyloxy)ethoxy]-1-adamantyl]methyl]-5-methyl-pyrazol-4-yl]pyridine-2-carboxylate

To the product from Step D (3.83 g, 4.48 mmol) and triethylamine (1.87 mL, 3 eq) in DCM (45 mL) was added p-tolylsulfonyl 4-methylbenzenesulfonate (2.19 g, 1.5 eq) and the reaction mixture was stirred for 2 h. Purification by column chromatography (silica gel, heptane and EtOAc as eluents) afforded 2.5 g (55%) of the desired product. ¹H NMR (400 MHz, DMSO-d₆): δ ppm 7.95 (d, 1H), 7.81 (brs, 1H), 7.76 (m, 2H), 7.45 (brs, 1H), 7.45 (m, 2H), 7.40 (s, 1H), 7.35 (m, 1H), 7.18 (m, 2H), 7.17 (m, 1H), 6.97 (d, 1H), 6.90 (m, 2H), 5.10 (s, 2H), 4.05 (m, 2H), 4.00 (m, 2H), 3.82 (s, 2H), 3.74 (s, 3H), 3.47 (m, 2H), 2.85 (m, 2H), 2.40 (s, 3H), 2.32 (s, 3H), 2.10 (s, 3H), 1.98 (m, 2H), 1.87-1.34 (m, 12H), 0.81 (s, 6H); HRMS-ESI (m/z): [M+H]⁺ calculated for C₅₅H₆₁N₈O₇S₂: 1009.4104 found: 1009.4102.

### Amine Substitution Procedure III

To the product from Preparation A for Precursors in a 1:1 mixture of acetonitrile and N-methyl-2-pyrrolidone (10 ml/mmol) was added the appropriate amine (3-10 eq) and the reaction mixture was stirred at 50° C. for 2-24 h. After the purification of the product by preparative reversed phase chromatography, the desired product was obtained.

### Precursor of P37: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-[2-[4-hydroxybutyl(methyl)amino]ethoxy]-5,7-dimethyl-1-adamantyl]methyl]-5-methyl-pyrazol-4-yl]pyridine-2-carboxylate

Using Amine substitution procedure III and 4-(methylamino) butan-1-ol as the appropriate amine, the desired product was obtained. HRMS-ESI (m/z): [M+H]⁺ calculated for C₅₃H₆₆N₉O₅S: 940.4907 found 940.4906.

### Precursor of P36: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-[2-[3-methoxypropyl(methyl)amino]ethoxy]-5,7-dimethyl-1-adamantyl]methyl]-5-methyl-pyrazol-4-yl]pyridine-2-carboxylate

Using Amine substitution procedure III and 3-methoxy-N-methyl-propan-1-amine as the appropriate amine, the desired product was obtained. HRMS-ESI (m/z): [M+H]⁺ calculated for C₅₃H₆₆N₉O₅S: 940.4907 found 940.4904.

### Precursor of P35: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-[2-[2-hydroxyethyl(methyl)amino]ethoxy]-5,7-dimethyl-1-adamantyl]methyl]-5-methyl-pyrazol-4-yl]pyridine-2-carboxylate

Using Amine substitution procedure III and 2-(methylamino) ethanol as the appropriate amine, the desired product was obtained. HRMS-ESI (m/z): [M+H]⁺ calculated for C₅₁H₆₂N₉O₅S: 912.4594 found 912.4592.

### Precursor of P27: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-[2-(dimethylamino)ethoxy]-5,7-dimethyl-1-adamantyl]methyl]-5-methyl-pyrazol-4-yl]pyridine-2-carboxylate

Using Amine substitution procedure III and dimethylamine as the appropriate amine, the desired product was obtained. HRMS-ESI (m/z): [M+H]⁺ calculated for C₅₀H₆₀N₉O₄S: 882.4489 found 882.4490.

### Precursor of P21: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3,5-dimethyl-7-(2-pyrrolidin-1-ylethoxy)-1-adamantyl]methyl]-5-methyl-pyrazol-4-yl]pyridine-2-carboxylate

Using Amine substitution procedure III and pyrrolidine as the appropriate amine, the desired product was obtained. HRMS-ESI (m/z): [M+2H]2+ calculated for C₅₂H₆₂N₉O₄S: 454.7362 found 454.7365.

### Precursor of P25: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-[2-(3-hydroxypropylamino)ethoxy]-5,7-dimethyl-1-adamantyl]methyl]-5-methyl-pyrazol-4-yl]pyridine-2-carboxylate

Using Amine substitution procedure III and 3-aminopropan-1-ol as the appropriate amine, the desired product was obtained. HRMS-ESI (m/z): [M+H]⁺ calculated for C₅₁H₆₂N₉O₅S: 912.4591, found 912.4581.

### Precursor of P19: (4-methoxyphenyl)methyl 6-[3-(1,3-benzothiazol-2-ylamino)-4-methyl-6,7-dihydro-5H-pyrido[2,3-c]pyridazin-8-yl]-3-[1-[[3-[2-[2-[(4S)-2,2-dimethyl-1,3-dioxolan-4-yl]ethylamino]ethoxy]-5,7-dimethyl-1-adamantyl]methyl]-5-methyl-pyrazol-4-yl]pyridine-2-carboxylate

Using Amine substitution procedure III and 2-[(4S)-2,2-dimethyl-1,3-dioxolan-4-yl]ethanamine as the appropriate amine, the desired product was obtained. HRMS-ESI (m/z): [M+H]⁺ calculated for C₅₅H₆₈N₉O₆S: 982.5013, found 982.5000.

### Example 3. Synthesis and Characterization of Linkers, Linker-Payloads, and Precursors Thereof

Exemplary linkers, linker-payloads, and precursors thereof were synthesized using exemplary methods described in this example.

Abbreviations:

| | |
|---|---|
| CuI | cupper (I) iodide |
| DCC | dicyclohexyl carbodiimide |
| DCM | dichloromethane |
| DEA | N-ethylethanamine |
| DIPEA: | N,N-Diisopropylethylamine |
| DMF: | dimethylformamide |
| DMSO: | dimethyl sulfoxide |
| EDC: | N-Ethyl,N′-dimethylamino-propylcarbodiimide |
| EEDQ | ethyl 2-ethoxy-2H-quinoline-1-carboxylate |
| Fmoc: | Fluorenylmethyloxycarbonyl |
| Fmoc- | (2S)-2-(9H-fluoren-9-ylmethoxycarbonylamino)-5- |
| Cit-OH | ureido-pentanoic acid |
| HBTU: | (2-(1 H-benzotriazol-1-yl)-1,1,3,3-tetrameth- |
| | yluronium hexafluorophosphate |
| HOAt: | 1-Hydroxy-7-azabenzotriazole |
| MgSO₄ | magnesium sulfate |
| MMAE: | (2S)-N-[(1S)-1-[(1S,2R)-4-[(2S)-2-[(1R,2R)- |
| | 3-[(1R,2S)-2-hydroxy-1-methyl-2-phenyl-ethyl]amino]- |
| | 1-methoxy-2-methyl-3-oxo-propyl]pyrrolidin-1- |
| | yl]-2-methoxy-1-[(1S)-1-methylpropyl]-4-oxo- |
| | butyl]-methyl-carbamoyl]-2-methyl-propyl]-3- |
| | methyl-2-(methylamino)butanamide (MMAE) |
| Na₂SO₄ | sodium sulfate |
| NH₄Cl | ammonium chloride |
| NMP | N-methylpyrrolidone |
| Pd(PPh₃)₂Cl₂ | dichloro-tri(triphenylphosphine)palladium |
| PBr₃ | tribromophosphane |
| Pt/C 10% | platinum over carbon 10% |
| RT | room temperature |
| SOCl₂ | thionyl chloride |
| THF | tetrahydrofuran |
| TBAF | tetrabutylammonium, fluoride |
| TBAI | tetrabutylammonium, iodide |
| TFA | trifluoroacetic acid |
| TSTU: | [dimethylamino-(2,5-dioxopyrrolidin-1-yl)oxy- |
| | methylene]-dimethyl-ammonium; tetrafluoroborate |

### Chemical Naming

IUPAC-preferred names were generated using the chemical naming functionality provided by Biovia® Draw 2018 (Version 18.1.NET).

### Materials, Methods & General Procedures

All reagents obtained from commercial sources were used without further purification. Anhydrous solvents were obtained from commercial sources and used without further drying. Flash chromatography was performed on CombiFlash R_{f} (Teledyne ISCO) with pre-packed silica-gel cartridges (Macherey-Nagel Chromabond Flash). Thin layer chromatography was conducted with 5×10 cm plates coated with Merck Type 60 F254 silica-gel. Microwave heating was performed in CEM Discover® instrument.

¹H-NMR measurements were performed on 400 MHz Bruker Avance or 500 MHz Avance Neo spectrometer, using DMSO-d₆ or CDCl₃ as solvent. ¹H NMR data is in the form of chemical shift values, given in part per million (ppm), using the residual peak of the solvent (2.50 ppm for DMSO-de and 7.26 ppm for CDCl₃) as internal standard. Splitting patterns are designated as: s (singlet), d (doublet), t (triplet), q (quartet), quint (quintet), m (multiplet), br s (broad singlet), br t (broad triplet) dd (doublet of doublets), td (triplet of doublets), dt (doublet of triplets), ddd (doublet of doublet of doublets). IR measurements were performed on a Bruker Tensor 27 equipped with ATR Golden Gate device (SPECAC). HRMS measurements were performed on a LTQ OrbiTrap Velos Pro mass spectrometer (ThermoFisher Scientific). Samples were dissolved in CH₃CN/H₂O (2/1: v/v) at a concentration range from 0.01 to 0.05 mg/ml approximately and introduced in the source by an injection of 2 μL in a flow of 0.1 mL/min. ESI ionization parameters were as follow: 3.5 kV and 350° C. transfer ion capillary. All the spectra were acquired in positive ion mode with a resolving power of 30,000 or 60,000 using a lock mass.

HRMS measurements were performed on an LTQ OrbiTrap Velos Pro mass spectrometer (ThermoFisher Scientific GmbH, Bremen, Germany). Samples were dissolved in CH₃CN/H₂O (2/1: v/v) at a concentration range from 0.01 to 0.05 mg/mL approximately and introduced in the source by an injection of 2 μL in a flow of 0.1 mL/min. ESI ionization parameters were as follows: 3.5 kV and 350° C. transfer ion capillary. All the spectra were acquired in positive ion mode with a resolving power of 30,000 or 60,000 using a lock mass. UPLC®-MS:

UPLC®-MS data were acquired using an instrument with the following parameters (Table 7):

TABLE 7
UPLC ®-MS Parameters

| | |
|---|---|
| Instrument(s) | Waters Aquity A-class with diode array UV |
| | detector “PDA” and “ZQ detector 2” mass |
| | device and MassLinks software. |
| ZQ detector 2 | MS scan from 0.15 to 6 min and from 100 to |
| | 2372 Da |
| PDA detector | from 190 to 400 nm |
| | Aquity UPLC ®BEH column C18, 1.7 μm, |
| | 130 Å, 2.1 × 50 mm |
| Columns | Column used at 40° C. with a flowrate of |
| | 0.6 mL/min |
| Solvent A | water + 0.02% TFA |
| Solvent B | acetonitrile + 0.02% TFA |
| Gradient | from 2% B to 100% B in 5 min, then 0.3 min |
| | washing with 100% B and 0.5 min equilibration |
| | at 2% B for the next injection (total gradient of |
| | 6 min). |

### Preparative-HPLC:

Preparative-HPLC (“Prep-HPLC”) data were acquired using an instrument with the following parameters (Table 8):

TABLE 8
Prep-HPLC Parameters

| | |
|---|---|
| Instrument(s) | Columns Waters X-Bridge 5 or 10 μm with |
| | sizes (flowrate) of: 19 × 50 mm (12 ml/min), |
| | 19 × 100 mm (12 ml/min), 30 × 100 mm (30-50 |
| | ml/min), 30 × 250 mm (30-50 ml/min), 50 × 250 |
| | mm (80-150 ml/min); |
| | Interchim Puriflash 4100 with a maximum of |
| | 100 bars and a maximum flowrate of 250 |
| | ml/min, or Interchim Puriflash 4250 with a |
| | maximum of 250 bars and a maximum |
| | flowrate of 250 ml/min; |
| UV | Quaternary solvent pump with the possibility to |
| | use 4 solvents at the same time in a gradient |
| | 2 wavelengths for the collection between 200 |
| | and 400 nm |
| Columns | Waters XBridge 10 μm |
| Collection | 8 ml or 32 ml tubes |

Three Prep-HPLC methods were used:
a. TFA method: solvent: A=water+0.05% TFA, B=acetonitrile+0.05% TFA, gradient from 5 to 100% B in 15 to 30 CV
b. NH₄HCO₃ method: solvent: A=water+0.02 M NH₄HCO₃, B=acetonitrile/water 80/20+0.02 M NH₄HCO₃, gradient from 5 to 100% B in 15 to 30 CV
c. Neutral method: solvent: A=water, B=acetonitrile, gradient from 5 to 100% B in 15 to 30 CV

All the fractions containing the pure compound were combined and directly freeze-dried to afford the compound as an amorphous powder.

### Method A

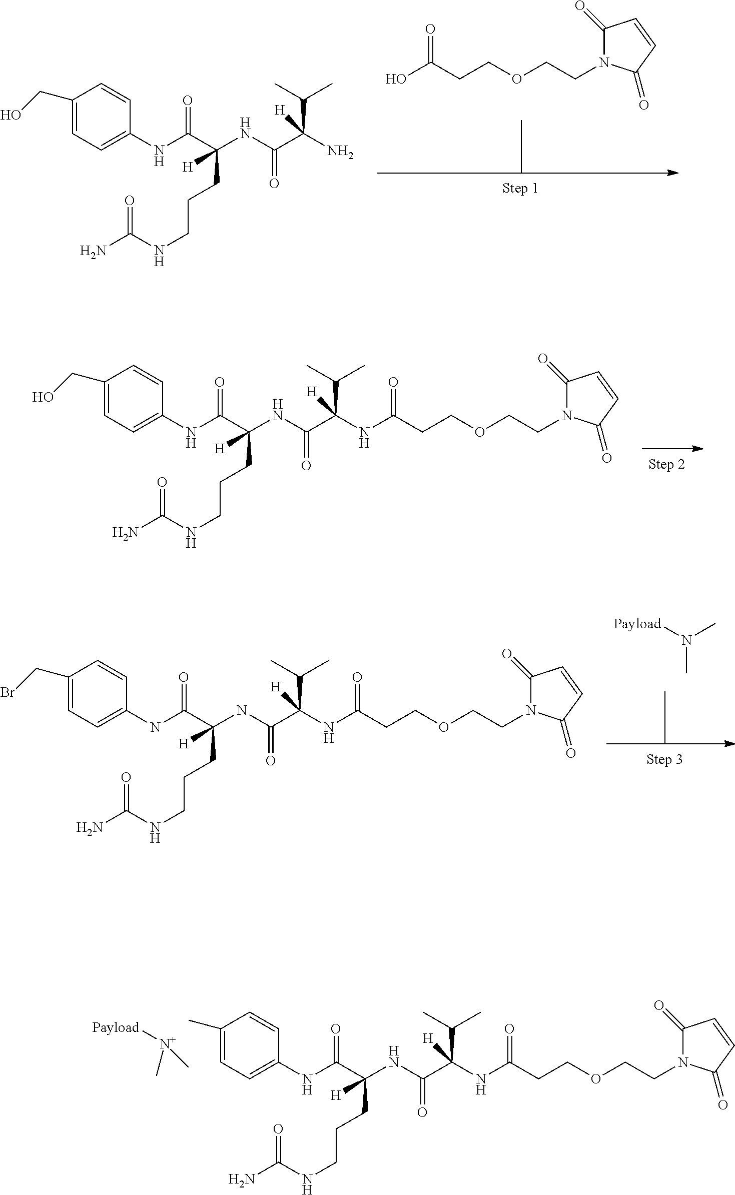

### Step 1: (2S)-2-[[(2S)-2-[3-[2-(2,5-dioxopyrrol-1-yl)ethoxy]propanoylamino]-3-methyl-butanoyl]amino]-N-[4-(hydroxymethyl)phenyl]-5-ureido-pentanamide

To a solution of 3-[2-(2,5-dioxopyrrol-1-yl)ethoxy]propanoic acid (855 mg, 4.01 mmol) in THF (42 mL) were added N,N′-dicyclohexylmethanediimine (1.05 g, 5.08 mmol) and 1-hydroxypyrrolidine-2,5-dione (510 mg, 4.43 mmol). The reaction mixture was stirred at room temperature for 20 h. The precipitate was removed by filtration and the filtrate was added to a solution of (2S)-2-[[(2S)-2-amino-3-methyl-butanoyl]amino]-N-[4-(hydroxymethyl)phenyl]-5-ureido-pentanamide (1.27 g, 3.35 mmol) in DMF (42 mL). The reaction mixture was stirred at room temperature for 20 h, diluted with diethyl ether (250 mL). The solid was recovered by filtration to afford (2S)-2-[[(2S)-2-[3-[2-(2,5-dioxopyrrol-1-yl)ethoxy]propanoylamino]-3-methyl-butanoyl]amino]-N-[4-(hydroxymethyl)phenyl]-5-ureido-pentanamide (1.81 g). ¹H NMR (400 MHz, dmso-d₆): δ 9.87 (s, 1H), 8.05 (d, 1H), 7.82 (d, 1H), 7.53 (d, 2H), 7.21 (d, 2H), 7.00 (s, 2H), 5.95 (t, 1H), 5.39 (s, 2H), 5.07 (t, 1H), 4.41 (d, 2H), 4.34-4.40 (m, 1H), 4.18-4.22 (m, 1H), 3.42-3.65 (m, 4H), 2.88-3.02 (m, 2H), 2.73 (s, 2H), 2.28-2.45 (m, 2H), 1.91-1.99 (m, 1H), 1.53-1.75 (m, 2H), 1.30-1.147 (m, 2H), 0.85 (d, 3H), 0.81 (d, 3H). ¹³C NMR (125 MHz, dmso-d₆): δ 171.05, 170.83, 170.32, 170.09, 158.82, 137.49, 137.37, 134.50, 126.88, 118.81, 66.66, 66.53, 62.57, 57.49, 53.06, 36.74, 35.76, 30.51, 29.31, 26.79, 25.20, 19.16, 18.07. MS (ESI) m/z [M+H]⁺=575.2.

### Step 2: (2S)—N-[4-(bromomethyl)phenyl]-2-[[(2S)-2-[3-[2-(2,5-dioxopyrrol-1-yl)ethoxy]propanoylamino]-3-methyl-butanoyl]amino]-5-ureido-pentanamide

To a solution of (2S)-2-[[(2S)-2-[3-[2-(2,5-dioxopyrrol-1-yl)ethoxy]propanoylamino]-3-methyl-butanoyl]amino]-N-[4-(hydroxymethyl)phenyl]-5-ureido-pentanamide (37.2 mg, 65 μmol) in THF (1 mL) was added dropwise phosphorus tribromide (45 μL, 97 mmol) at 0° C. under argon. The reaction was stirred at 0° C. for 1 h and at room temperature for 2 h. The progress of the reaction was followed by UPLC-MS: an aliquot was treated by a large excess of morpholine in acetonitrile, following the formation of the corresponding morpholine adduct. The reaction was diluted with THF (3 mL), quenched by the addition of 2 drops of a saturated solution of NaHCO₃, stirred for 5 min at room temperature, dried over magnesium sulfate and filtered. The residue, containing the crude (2S)—N-[4-(bromomethyl)phenyl]-2-[[(2S)-2-[3-[2-(2,5-dioxopyrrol-1-yl)ethoxy]propanoylamino]-3-methyl-butanoyl]amino]-5-ureido-pentanamide (45 mg) was used immediately in the next step. MS (ESI) m/z [M+H]⁺=662.62 (morpholine adduct).

### Step 3: General Procedure for Linker Introduction

To a suspension of the payload (19.6 μmol) in DMF (30 mL/mmol) was added a solution of the product of Step 2 (1.2 eq.) in THF (50 mL/mmol) and DIPEA (3 eq.). The reaction was stirred at room temperature for 2 h. The crude product was purified using C18 reverse phase prep-HPLC by direct deposit of the reaction mixture on the Xbridge® column and using the TFA method to give the desired compound.

### Preparation of L9A-P27: 2-[[(5R,7S)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]oxy]ethyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-dimethyl-ammonium;2,2,2-trifluoroacetate

Using Method A and P27 as the appropriate payload, the desired product was obtained. HRMS (ESI) [M−CF₃CO₂]⁺=1318.6557 (δ=0.2 ppm)

### Preparation of L9A-P30: 2-[[(5RS,7SR)-3-[[4-[6-[[6-(1,3-benzothiazol-2-ylamino)-5-methyl-pyridazin-3-yl]-methyl-amino]-2-carboxy-3-pyridyl]-5-methyl-pyrazol-1-yl]methyl]-5,7-dimethyl-1-adamantyl]oxy]ethyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-dimethyl-ammonium;2,2,2-trifluoroacetic acid

Using Method A and P30 as the appropriate payload, the desired product was obtained. HRMS (ESI) [M−CF₃CO₂]⁺=1292.6386 (δ=−0.9 ppm).

### Preparation of L9A-P33: 6-[3-(1,3-benzothiazol-2-ylamino)-4-methyl-6,7-dihydro-5H-pyrido[2,3-c]pyridazin-8-yl]-3-[1-[[(5RS,7SR)-3-[2-[1-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]azepan-1-ium-1-yl]ethoxy]-5,7-dimethyl-1-adamantyl]methyl]-5-methyl-pyrazol-4-yl]pyridine-2-carboxylic acid;2,2,2-trifluoroacetic acid

Using Method A and P33 as the appropriate payload, the desired product was obtained. HRMS (ESI) [M−CF₃CO₂]⁺ found=1372.7019 (δ=−0.3 ppm).

### Preparation of L9A-P32: 6-[3-(1,3-benzothiazol-2-ylamino)-4-methyl-6,7-dihydro-5H-pyrido[2,3-c]pyridazin-8-yl]-3-[1-[[(5RS,7SR)-3-[2-[4-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-4-isopropyl-piperazin-4-ium-1-yl]ethoxy]-5,7-dimethyl-1-adamantyl]methyl]-5-methyl-pyrazol-4-yl]pyridine-2-carboxylic acid;2,2,2-trifluoroacetic acid

Using Method A and P32 as the appropriate payload, the desired product was obtained. HRMS (ESI) [M−CF₃CO₂]⁺=1401.7287 (δ=−0.1 ppm).

### Preparation of L9A-P38: 6-[3-(1,3-benzothiazol-2-ylamino)-4-methyl-6,7-dihydro-5H-pyrido[2,3-c]pyridazin-8-yl]-3-[1-[[(5RS,7SR)-3-[2-[1-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]piperidin-1-ium-1-yl]ethoxy]-5,7-dimethyl-1-adamantyl]methyl]-5-methyl-pyrazol-4-yl]pyridine-2-carboxylic acid;2,2,2-trifluoroacetic acid

Using Method A and P38 as the appropriate payload, the desired product was obtained. HRMS (ESI) [M−CF₃CO₂]⁺=1358.6803 (δ=−4.7 ppm).

### Preparation of L9A-P39: 6-[3-(1,3-benzothiazol-2-ylamino)-4-methyl-6,7-dihydro-5H-pyrido[2,3-c]pyridazin-8-yl]-3-[1-[[(5SR,7RS)-3-[2-[4-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]morpholin-4-ium-4-yl]ethoxy]-5,7-dimethyl-1-adamantyl]methyl]-5-methyl-pyrazol-4-yl]pyridine-2-carboxylate;2,2,2-trifluoroacetic acid

Using Method A and P39 as the appropriate payload, the desired product was obtained. HRMS (ESI) [M+H]⁺ found=1360.6634 (δ=−1.9 ppm).

### Preparation of L9A-P41: 6-[3-(1,3-benzothiazol-2-ylamino)-4-methyl-6,7-dihydro-5H-pyrido[2,3-c]pyridazin-8-yl]-3-[1-[[(5SR,7RS)-3-[3-[1-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]pyrrolidin-1-ium-1-yl]propyl]-5,7-dimethyl-1-adamantyl]methyl]-5-methyl-pyrazol-4-yl]pyridine-2-carboxylic acid;2,2,2-trifluoroacetic acid

Using Method A and P41 as the appropriate payload, the desired product was obtained. HRMS (ESI) [M−CF₃CO₂]⁺ found=1342.6844 (δ=−5.5 ppm).

### Preparation of L9A-P42: 3-[1-[[(5RS,7SR)-3-[2-[1-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]pyrrolidin-1-ium-1-yl]ethoxy]-5,7-dimethyl-1-adamantyl]methyl]-5-methyl-pyrazol-4-yl]-6-[4-methyl-3-[(5-methyl-1,3-benzothiazol-2-yl)amino]-6,7-dihydro-5H-pyrido[2,3-c]pyridazin-8-yl]pyridine-2-carboxylic acid;2,2,2-trifluoroacetic acid

Using Method A and P42 as the appropriate payload, the desired product was obtained. HRMS (ESI) [M−CF₃CO₂]⁺ found=1358.6807 (δ=−4.4 ppm).

### Method B

### Step 1:

To a suspension of the para methoxy benzyl (PMB)-protected payload (11.3 μmol) in DMF (0.4 mL) was added a solution of (2S)—N-[4-(bromomethyl)phenyl]-2-[[(2S)-2-[3-[2-(2,5-dioxopyrrol-1-yl)ethoxy]propanoylamino]-3-methyl-butanoyl]amino]-5-ureido-pentanamide (12.4 mg, 13.6 μmol) in THF (0.2 mL) and DIPEA (9.8 μL, 56.7 μmol). The reaction was stirred at room temperature for 4 h. The crude product was purified using C18 reverse phase prep-HPLC by direct deposit of the reaction mixture on the Xbridge® column and using the TFA method to afford the expected compound which was directly used in Step 2.

### Step 2:

To a suspension of the product from Step 1 in DCM (3.2 mL) was added TFA (320 μL, 4.18 mmol). The reaction was stirred at room temperature for 1 h. The solvent was evaporated and the residue dissolved in DMF (500 μL) This crude solution was purified using C18 reverse phase prep-HPLC by direct deposit of the reaction mixture on the Xbridge® column and using the TFA method to afford the desired product.

### Preparation of L9A-P35: 2-[[(5RS,7SR)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]oxy]ethyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-(2-hydroxyethyl)-methyl-ammonium;2,2,2-trifluoroacetic acid

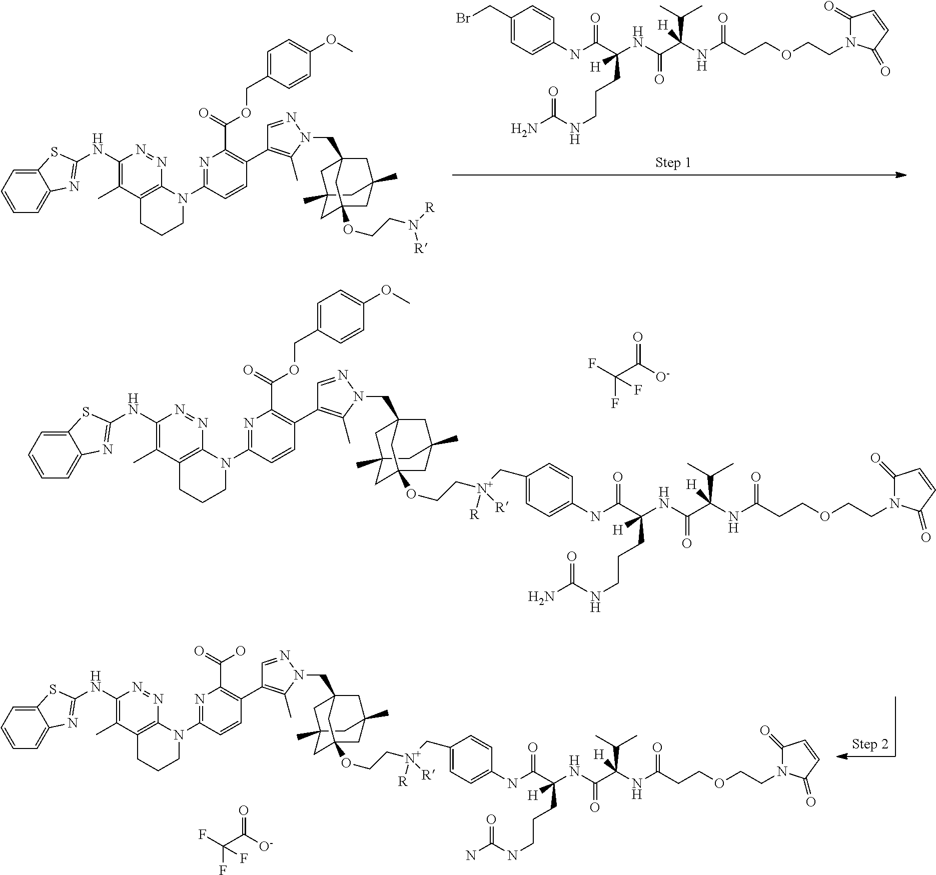

Using Method B and the precursor of P35 as the appropriate PMB-protected payload, the desired product was obtained. HRMS (ESI) [M−CF₃CO₂]⁺ found=1318.6531 (δ=−1.7 ppm).

### Preparation of L9A-P36: 2-[[(5RS,7SR)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]oxy]ethyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-(3-methoxypropyl)-methyl-ammonium;2,2,2-trifluoroacetic acid

Using Method B and the precursor of P36 as the appropriate PMB-protected payload, the desired product was obtained. HRMS (ESI) [M−CF₃CO₂]⁺ found=1376.6930 (δ=−3.1 ppm).

### Preparation of L9A-P37: 2-[[(5SR,7RS)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]oxy]ethyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-(4-hydroxybutyl)-methyl-ammonium;2,2,2-trifluoroacetic acid

Using Method B and the precursor of P37 as the appropriate PMB-protected payload, the desired product was obtained. HRMS (ESI) [M−CF₃CO₂]⁺ found=1376.6918 (δ=−3.9 ppm).

### Method C

### Preparation of L9C-P19: 6-[3-(1,3-benzothiazol-2-ylamino)-4-methyl-6,7-dihydro-5H-pyrido[2,3-c]pyridazin-8-yl]-3-[1-[[(5SR,7RS)-3-[2-[[(3S)-3,4-dihydroxybutyl]-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl]amino]ethoxy]-5,7-dimethyl-1-adamantyl]methyl]-5-methyl-pyrazol-4-yl]pyridine-2-carboxylic acid

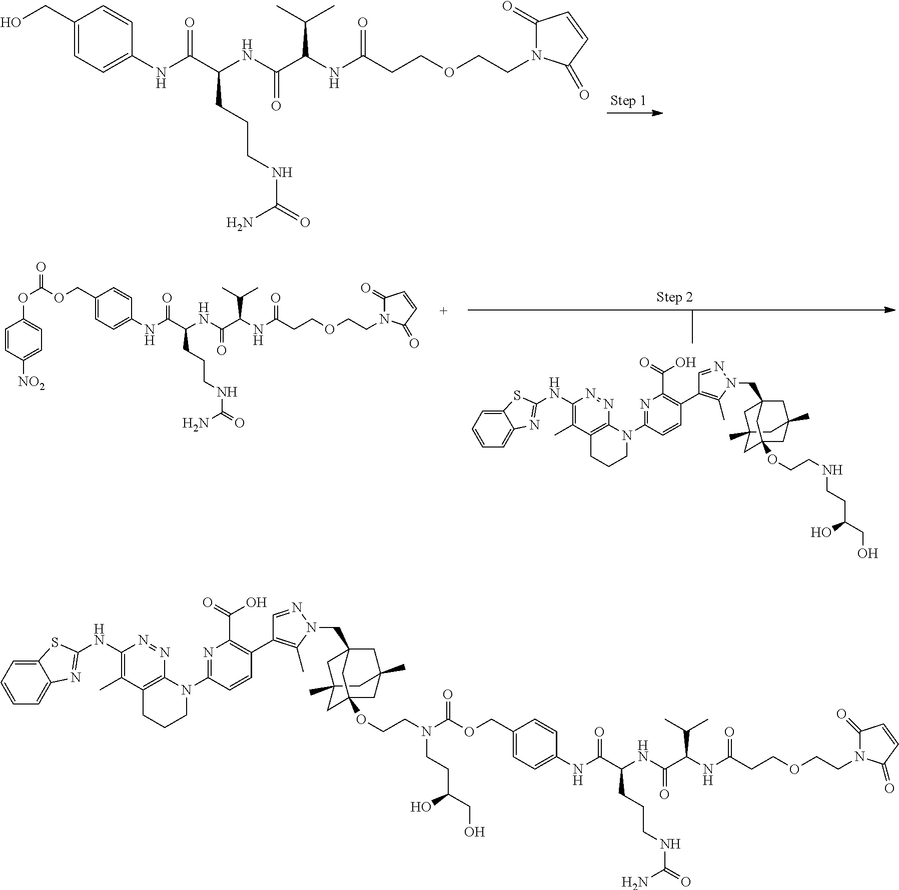

### Step 1: [4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl (4-nitrophenyl) carbonate

To a solution of (2S)-2-[[(2S)-2-[3-[2-(2,5-dioxopyrrol-1-yl)ethoxy]propanoylamino]-3-methyl-butanoyl]amino]-N-[4-(hydroxymethyl)phenyl]-5-ureido-pentanamide (from Method A, Step 1)(580 mg; 1.0 mmol) in dry DMF were added DIPEA (0.5 mL; 3.025 mmol; 3 eq.) and bis(4-nitrophenyl) carbonate (615 mg; 2.02 mmol; 2 eq.). The reaction mixture was stirred at room temperature for 68 h. The reaction mixture was diluted with diethyl ether (15 mL) and the solid was filtered to afford the title compound (589 mg; 79%). ¹H NMR (dmso-d₆): 0.82 (d, 3H, J=6.8 Hz), 0.85 (d, 3H, J=6.8 Hz), 1.47-1.33 (m, 2H), 1.74-1.54 (m, 2H), 1.92-2.00 (m, 1H), 2.32-2.45 (m, 2H), 2.90-3.06 (m, 2H), 3.49-3.46 (m, 2H), 3.60-3.52 (m, 4H), 4.21 (dd, 1H, J=8.7 and 6.8 Hz), 4.39 (m, 1H), 5.24 (s, 2H), 5.39 (s, 2H), 5.96 (t, 1H, J=5.6 Hz), 7.00 (s, 2H), 7.41 (d, 2H, J=8.8 Hz), 7.57 (dd, 2H, J=6.8 and 2.4 Hz), 7.65 (d, 2H, J=8.4 Hz), 7.83 (d, 1H, J=8.8 Hz), 8.10 (d, 1H, J=7.6 Hz), 8.31 (dd, 2H, J=6.8 and 2.4 Hz), 10.03 (s, 1H). LCMS Positive mode 740.14 detected (M+H⁺).

### Step 2: 6-[3-(1,3-benzothiazol-2-ylamino)-4-methyl-6,7-dihydro-5H-pyrido[2,3-c]pyridazin-8-yl]-3-[1-[[(5SR,7RS)-3-[2-[[(3S)-3,4-dihydroxybutyl]-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl]amino]ethoxy]-5,7-dimethyl-1-adamantyl]methyl]-5-methyl-pyrazol-4-yl]pyridine-2-carboxylic acid

To a suspension of P19 (15 mg, 0.016 mmol) in DMF (0.5 mL) were added DIPEA (14 μL, 0.0801 mmol) and the carbonate of Step 1 (14.2 mg, 0.0192 mmol) and the mixture was stirred at room temperature for 18 h. The crude product was purified using C18 reverse phase prep-HPLC by direct deposit of the reaction mixture on the Xbridge® column and using the TFA method to afford the title compound (6.9 mg, yield 30%). ¹H NMR (500 MHz, dmso-d₆) δ ppm (m, 2H), (m, 4H), (m, 10H), (m, 2H), 9.98 (s), 8.08 (d), 7.9 (d, 1H), 7.82 (d), 7.8 (large, 1H), 7.79 (largeNC, 1H), 7.6 (m, 2H), 7.49 (largeNC, 1H), 7.43 (br s, 1H), 7.37 (t, 1H), 7.28 (d, 2 H), 7.19 (t, 1H), 7 (s, 2H), 5.97 (br s), 5.42 (large), 4.99 (s, 2H), 4.38 (m, 1H), 4.22 (t, 1H), 4.03 (t, 2H), 3.86 (m, 2H), 3.57/3.46/3.28/3.21 (m, 6H), 3.53 (m, 2H), 3.42 (m, 2H), 3.38 (m, 1H), 3.01/2.94 (2m, 2H), 2.89 (t, 2H), 2.43/2.32 (2m, 2H), 2.37 (s, 3H), 2.2 (s, 3H), 2.03 (m, 2H), 1.95 (m, 1H), 1.7/1.38 (2m, 2H), 0.84 (m, 6H), 0.84 (m, 6H). ¹³C NMR (500 MHz, dmso-d₆) δ ppm 137.6, 135.5, 128.7, 126.8, 122.7, 122.1, 119.1, 118.4, 69.7, 66.9, 66.2, 58.9, 58.4, 58.3, 53.7, 50.5/47.1/43.5, 48.3/46, 46, 39, 36.9, 36.6, 32.8, 30.9, 30.5, 30, 27.7, 24.4, 21.3, 19.8, 13.5, 10.8. HRMS (ESI) [M+H]⁺ found=1422.6688 (δ=1.6 ppm)

### Preparation of L9C-P22: 6-[3-(1,3-benzothiazol-2-ylamino)-4-methyl-6,7-dihydro-5H-pyrido[2,3-c]pyridazin-8-yl]-3-[1-[[(5RS,7SR)-3-[2-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-(4-hydroxybutyl)amino]ethoxy]-5,7-dimethyl-1-adamantyl]methyl]-5-methyl-pyrazol-4-yl]pyridine-2-carboxylic acid;2,2,2-trifluoroacetic acid

Using Method C and P22 as the appropriate payload, the desired product was obtained. HRMS (ESI) [M+H]⁺ found=1406.6728 (δ=1.0 ppm).

### Preparation of L9C-P23: 6-[3-(1,3-benzothiazol-2-ylamino)-4-methyl-6,7-dihydro-5H-pyrido[2,3-c]pyridazin-8-yl]-3-[1-[[(5SR,7RS)-3-[2-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-[3-hydroxy-2-(hydroxymethyl)propyl]amino]ethoxy]-5,7-dimethyl-1-adamantyl]methyl]-5-methyl-pyrazol-4-yl]pyridine-2-carboxylic acid;2,2,2-trifluoroacetic acid

Using Method C and P23 as the appropriate payload, the desired product was obtained. HRMS (ESI) [M+H]⁺ found=1422.6670 (δ=0.5 ppm).

### Preparation of L9C-P24: 6-[3-(1,3-benzothiazol-2-ylamino)-4-methyl-6,7-dihydro-5H-pyrido[2,3-c]pyridazin-8-yl]-3-[1-[[(5RS,7SR)-3-[2-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-[2-hydroxy-1-(hydroxymethyl)ethyl]amino]ethoxy]-5,7-dimethyl-1-adamantyl]methyl]-5-methyl-pyrazol-4-yl]pyridine-2-carboxylic acid;2,2,2-trifluoroacetic acid

Using Method C and P24 as the appropriate payload, the desired product was obtained. HRMS (ESI) [M+H]⁺ found=1408.6518 (δ=0.8 ppm).

### Preparation of L9C-P25: 6-[3-(1,3-benzothiazol-2-ylamino)-4-methyl-6,7-dihydro-5H-pyrido[2,3-c]pyridazin-8-yl]-3-[1-[[(5SR,7RS)-3-[2-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-(3-hydroxypropyl)amino]ethoxy]-5,7-dimethyl-1-adamantyl]methyl]-5-methyl-pyrazol-4-yl]pyridine-2-carboxylic acid;2,2,2-trifluoroacetic acid

Using Method C and P25 as the appropriate payload, the desired product was obtained. HRMS (ESI) [M+H]⁺ found=1366.6396 (δ=−0.4 ppm).

### Preparation of L9C-P26: 6-[[6-(1,3-benzothiazol-2-ylamino)-5-methyl-pyridazin-3-yl]-methyl-amino]-3-[1-[[(5SR,7RS)-3-[2-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-(3-hydroxypropyl)amino]ethoxy]-5,7-dimethyl-1-adamantyl]methyl]-5-methyl-pyrazol-4-yl]pyridine-2-carboxylic acid;2,2,2-trifluoroacetic acid

Using Method C and P26 as the appropriate payload, the desired product was obtained. HRMS (ESI) [M+H]⁺ found=1366.6396 (δ=−0.4 ppm).

### Preparation of L9C-P29: 6-[[6-(1,3-benzothiazol-2-ylamino)-5-methyl-pyridazin-3-yl]-methyl-amino]-3-[1-[[(5RS,7SR)-3-[2-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-(3-methoxypropyl)amino]ethoxy]-5,7-dimethyl-1-adamantyl]methyl]-5-methyl-pyrazol-4-yl]pyridine-2-carboxylic acid;2,2,2-trifluoroacetic acid

Using Method C and P29 as the appropriate payload, the desired product was obtained. HRMS (ESI) [M+H]⁺ found=1380.6575 (δ=1.2 ppm).

### Preparation of L9C-P31: 6-[3-(1,3-benzothiazol-2-ylamino)-4-methyl-6,7-dihydro-5H-pyrido[2,3-c]pyridazin-8-yl]-3-[1-[[(5RS,7SR)-3-[2-[4-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl]piperazin-1-yl]ethoxy]-5,7-dimethyl-1-adamantyl]methyl]-5-methyl-pyrazol-4-yl]pyridine-2-carboxylic acid;2,2,2-trifluoroacetic acid

Using Method C and P31 as the appropriate payload, the desired product was obtained. HRMS (ESI) [M+H]⁺ found=1403.6694 (δ=1.7 ppm).

### Preparation of L9C-P40: 6-[3-(1,3-benzothiazol-2-ylamino)-4-methyl-6,7-dihydro-5H-pyrido[2,3-c]pyridazin-8-yl]-3-[1-[[(5RS,7SR)-3-[3-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-(3-hydroxypropyl)amino]propyl]-5,7-dimethyl-1-adamantyl]methyl]-5-methyl-pyrazol-4-yl]pyridine-2-carboxylic acid;2,2,2-trifluoroacetic acid

Using Method C and P40 as the appropriate payload, the desired product was obtained. HRMS (ESI) [M+H]⁺ found=1390.6775 (δ=0.7 ppm).

### Preparation of L9A-P43: 3-[1-[[(5RS,7SR)-3-[2-[1-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]pyrrolidin-1-ium-1-yl]ethoxy]-5,7-dimethyl-1-adamantyl]methyl]-5-methyl-pyrazol-4-yl]-6-[3-[(5-methoxy-1,3-benzothiazol-2-yl)amino]-4-methyl-6,7-dihydro-5H-pyrido[2,3-c]pyridazin-8-yl]pyridine-2-carboxylic acid;2,2,2-trifluoroacetic acid

Using Method A and P43 as the appropriate payload, the desired product was obtained. HRMS (ESI) [M−CF₃CO₂]⁺ found=1374.6754 (δ=−4.5 ppm).

### Method D

### Preparation of L9A-P20: 6-[3-(1,3-benzothiazol-2-ylamino)-4-methyl-6,7-dihydro-5H-pyrido[2,3-c]pyridazin-8-yl]-3-[1-[[(5RS,7SR)-3-[2-[4-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-4-methyl-piperazin-4-ium-1-yl]ethoxy]-5,7-dimethyl-1-adamantyl]methyl]-5-methyl-pyrazol-4-yl]pyridine-2-carboxylic acid;2,2,2-trifluoroacetic acid

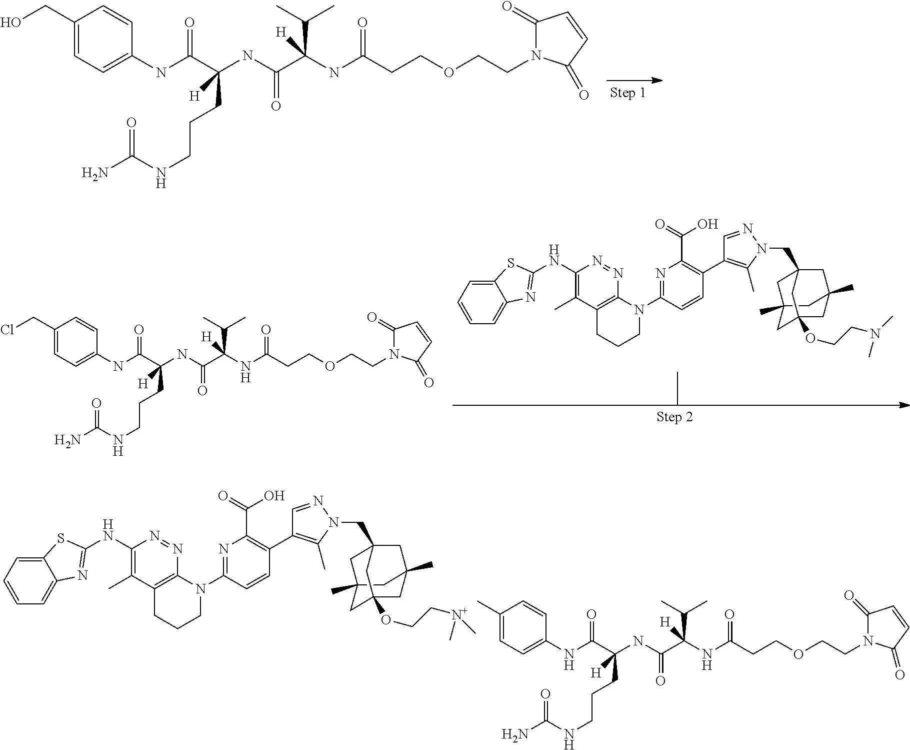 [SMILES: CC(C)[C@@H](NC(=O)CCOCCN1C(=O)C=CC1=O)C(=O)N[C@@H](CCCNC(N)=O)C(=O)Nc1ccc(CO)cc1 ; CC(C)[C@@H](NC(=O)CCOCCN1C(=O)C=CC1=O)C(=O)N[C@@H](CCCNC(N)=O)C(=O)Nc1ccc(CCl)cc1 ; Cc1c(Nc2nc3ccccc3s2)nnc2c1CCCN2c1ccc(-c2cnn(C[C@@]34C[C@]5(C)C[C@](C)(C3)C[C@](OCCN(C)C)(C5)C4)c2C)c(C(=O)O)n1 ; Cc1c(Nc2nc3ccccc3s2)nnc2c1CCCN2c1ccc(-c2cnn(C[C@@]34C[C@]5(C)C[C@](C)(C3)C[C@](OCC[NH+](C)C)(C5)C4)c2C)c(C(=O)O)n1 ; Cc1ccc(NC(=O)[C@H](CCCNC(N)=O)NC(=O)[C@H](NC(=O)CCOCCN2C(=O)C=CC2=O)C(C)C)cc1]

### Step 1: (2S)—N-[4-(chloromethyl)phenyl]-2-[[(2S)-2-[3-[2-(2,5-dioxopyrrol-1-yl)ethoxy]propanoylamino]-3-methyl-butanoyl]amino]-5-ureido-pentanamide

A solution of SOCl₂ (102 μL, 1.39 mmol) in THF (8 ml) was prepared as Solution A. A solution of (2S)-2-[[(2S)-2-[3-[2-(2,5-dioxopyrrol-1-yl)ethoxy]propanoylamino]-3-methyl-butanoyl]amino]-N-[4-(hydroxymethyl)phenyl]-5-ureido-pentanamide (from Method A, Step 1) (100 mg, 0.174 mmol) in THF (4 ml) was prepared as Solution B. Then 500 μl of Solution A was added every 10 min to Solution B. The reaction was followed by UPLC-MS after addition of morpholine in the sample. After completion of the reaction, the mixture was evaporated under reduced pressure at room temperature and directly used in the next step (105 mg, 0.177 mmol). ¹H NMR (400 MHz, dmso-d₆) δ ppm 10.00 (s, 1H), 8.10 (d, 1H), 7.85 (d, 1H), 7.60 (d, 2H), 7.35 (d, 2H), 7.00 (s, 2H), 6.05 (m, 1H), 5.25 (m, 2H), 4.70 (s, 2H), 4.40 (m, 1H), 4.20 (m, 1H), 3.65-3.40 (m, 6H), 3.00 (2m, 2H), 2.4/2.3 (2m, 2H), 2.00 (m, 1H), 1.7/1.6 (2m, 2H), 1.40 (2m, 2H), 0.80 (2d, 6H). IR: (v cm−1) 3288, 1703, 1643. HR-ESI+: [M+H]⁺=found 593.2499 (δ=2.4 ppm).

### Step 2:6-[3-(1,3-benzothiazol-2-ylamino)-4-methyl-6,7-dihydro-5H-pyrido[2,3-c]pyridazin-8-yl]-3-[1-[[(5RS,7SR)-3-[2-[4-[[4-[[(2S)-2-[[(2S)-2-[3-[2-(2,5- dioxopyrrol-1-yl)ethoxy]propanoylamino]-3-methyl-butanoyl]amino]-5-ureido-pentanoyl]amino]phenyl]methyl]-4-methyl-piperazin-4-ium-1-yl]ethoxy]-5,7-dimethyl-1-adamantyl]methyl]-5-methyl-pyrazol-4-yl]pyridine-2-carboxylic acid;2,2,2-trifluoroacetic acid

To a solution of P20 (15 mg, 14.4 μmol) in DMF (0.5 mL) was added a solution of the product from Step 1 (14.6 mg, 17.2 μmol) and DIPEA (8 μL, 43.1 μmol). The reaction was stirred at 80° C. for 18 h. The crude product was purified using C18 reverse phase prep-HPLC by direct deposit of the reaction mixture on the column and using the TFA method to afford the title compound (19.0 mg, yield 96%). HRMS (ESI) [M]⁺ found=1373.6974 (δ=−0.1 ppm).

### Preparation of L9A-P21: 6-[3-(1,3-benzothiazol-2-ylamino)-4-methyl-6,7-dihydro-5H-pyrido[2,3-c]pyridazin-8-yl]-3-[1-[[(5RS,7SR)-3-[2-[1-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]pyrrolidin-1-ium-1-yl]ethoxy]-5,7-dimethyl-1-adamantyl]methyl]-5-methyl-pyrazol-4-yl]pyridine-2-carboxylic acid;2,2,2-trifluoroacetic acid

Using Method D and P21 and as the appropriate payload, the desired product was obtained. HRMS (ESI) [M]⁺ found=1344.6688 (δ=−1.7 ppm).

### Preparation of L9A-P2: 2-[[6-(1,3-benzothiazol-2-ylamino)-5-methyl-pyridazin-3-yl]-methyl-amino]-5-[3-[4-[3-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl-dimethyl-ammonio]prop-1-ynyl]-2-fluoro-phenoxy]propyl]thiazole-4-carboxylate;2,2,2-trifluoroacetic acid

Using Method A and P2 as the appropriate payload, the desired product was obtained. HRMS (ESI) [M+H]⁺=1188.4561 (δ=0.6 ppm).

### Preparation of L9A-P1: 3-[4-[3-[2-[3-(1,3-benzothiazol-2-ylamino)-4-methyl-6,7-dihydro-5H-pyrido[2,3-c]pyridazin-8-yl]-4-carboxy-thiazol-5-yl]propoxy]-3-fluoro-phenyl]prop-2-ynyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-dimethyl-ammonium;2,2,2-trifluoroacetic acid

Using Method A and P1 as the appropriate payload, the desired product was obtained. HRMS (ESI) [M+H]⁺=1232.4802 (δ=−1.1 ppm).

### Preparation of L9A-P10: 2-[3-(1,3-benzothiazol-2-ylamino)-4-methyl-6,7-dihydro-5H-pyrido[2,3-c]pyridazin-8-yl]-5-[3-[4-[3-[4-[[4-[[(2S)-2-[[(2S)-2-[3-[2-(2,5- dioxopyrrol-1-yl)ethoxy]propanoylamino]-3-methyl-butanoyl]amino]-5-ureido-pentanoyl]amino]phenyl]methyl]-4-methyl-piperazin-4-ium-1-yl]prop-1-ynyl]-2-fluoro-phenoxy]propyl]thiazole-4-carboxylate;2,2,2-trifluoroacetic acid

Using Method A and P10 as the appropriate payload, the desired product was obtained. HRMS (ESI) [M+H]⁺ found=1269.5176 (δ=3.4 ppm).

### Preparation of L9A-P9: 2-[3-(1,3-benzothiazol-2-ylamino)-4-methyl-6,7-dihydro-5H-pyrido[2,3-c]pyridazin-8-yl]-5-[3-[4-[3-[1-[[4-[[(2S)-2-[[(2S)-2-[3-[2-(2,5- dioxopyrrol-1-yl)ethoxy]propanoylamino]-3-methyl-butanoyl]amino]-5-ureido-pentanoyl]amino]phenyl]methyl]pyrrolidin-1-ium-1-yl]prop-1-ynyl]-2-fluoro-phenoxy]propyl]thiazole-4-carboxylate;2,2,2-trifluoroacetic acid

Using Method A and P9 as the appropriate payload, the desired product was obtained. HRMS (ESI) [M+H]⁺ found=1240.4887 (δ=1.6 ppm).

### Preparation of L9A-P15: 2-[3-(1,3-benzothiazol-2-ylamino)-4-methyl-6,7-dihydro-5H-pyrido[2,3-c]pyridazin-8-yl]-5-[3-[4-[3-[1-[[4-[[(2S)-2-[[(2S)-2-[3-[2-(2,5- dioxopyrrol-1-yl)ethoxy]propanoylamino]-3-methyl-butanoyl]amino]-5-ureido-pentanoyl]amino]phenyl]methyl]-4,4-difluoro-piperidin-1-ium-1-yl]prop-1-ynyl]-2-fluoro-phenoxy]propyl]thiazole-4-carboxylate;2,2,2-trifluoroacetic acid

Using Method A and P15 as the appropriate payload, the desired product was obtained. HRMS (ESI) [M+H]⁺ found=1290.4831 (δ=−0.3 ppm).

### Preparation of L9A-P18: 2-[3-(1,3-benzothiazol-2-ylamino)-4-methyl-6,7-dihydro-5H-pyrido[2,3-c]pyridazin-8-yl]-5-[3-[4-[3-[1-[[4-[[(2S)-2-[[(2S)-2-[3-[2-(2,5- dioxopyrrol-1-yl)ethoxy]propanoylamino]-3-methyl-butanoyl]amino]-5-ureido-pentanoyl]amino]phenyl]methyl]piperidin-1-ium-1-yl]prop-1-ynyl]-2-fluoro-phenoxy]propyl]thiazole-4-carboxylate; 2,2,2-trifluoroacetic acid

Using Method A and P18 as the appropriate payload, the desired product was obtained. HRMS (ESI) [M+H]⁺ found=1254.4990 (δ=−1.8 ppm).

### Preparation of L9A-P28: 3-[4-[3-[2-[3-(1,3-benzothiazol-2-ylamino)-4-methyl-6,7-dihydro-5H-pyrido[2,3-c]pyridazin-8-yl]-4-carboxy-thiazol-5-yl]propoxy]phenyl]prop-2-ynyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-dimethyl-ammonium; 2,2,2-trifluoroacetic acid

Using Method A and P28 as the appropriate payload, the desired product was obtained. HRMS (ESI) [M−CF₃CO₂]⁺=1196.4827 (δ=1.9 ppm).

### Preparation of L9C-P16: 2-[3-(1,3-benzothiazol-2-ylamino)-6-[2-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-methyl-amino]ethoxy]-4-methyl-6,7-dihydro-5H-pyrido[2,3-c]pyridazin-8-yl]-5-[3-[4-[3-(dimethylamino)prop-1-ynyl]-2-fluoro-phenoxy]propyl]thiazole-4-carboxylic acid

Using Method C and P16 as the appropriate payload, the desired product was obtained. HRMS (ESI) [M+H]⁺ found=1331.5131 (δ=−0.4 ppm).

### Preparation of L9C-P12: 2-[3-(1,3-benzothiazol-2-ylamino)-4-methyl-6,7-dihydro-5H-pyrido[2,3-c]pyridazin-8-yl]-5-[3-[4-[3-[2-(dimethylamino)ethyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl]amino]prop-1-ynyl]-2-fluoro-phenoxy]propyl]thiazole-4-carboxylic acid

Using Method C and P12 as the appropriate payload, the desired product was obtained. HRMS (ESI) [M+H]⁺ found=1301.5034 (δ=−0.3 ppm).

### Preparation of L9C-P44: 2-[[6-(1,3-benzothiazol-2-ylamino)-5-methyl-pyridazin-3-yl]-(3,4-dihydroxybutyl)amino]-5-[3-[4-[3-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-methyl-amino]prop-1-ynyl]-2-fluoro-phenoxy]propyl]thiazole-4-carboxylic acid

Using Method C and P44 as the appropriate payload, the desired product was obtained. HRMS (ESI) [M+H]⁺ found=1262.4527 (δ=−0.1 ppm).

### Preparation of L9C-P45: 2-[[6-(1,3-benzothiazol-2-ylamino)-5-methyl-pyridazin-3-yl]-(3-hydroxypropyl)amino]-5-[3-[4-[3-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-methyl-amino]prop-1-ynyl]-2-fluoro-phenoxy]propyl]thiazole-4-carboxylic acid

Using Method C and P45 as the appropriate payload, the desired product was obtained after a purification step based on the NH₄HCO₃ method (Prep-HPLC, general procedures). HRMS (ESI) [M+H]⁺ found=1262.4527 (δ=0.4 ppm).

### Preparation of L9C-P46: 2-[[6-(1,3-benzothiazol-2-ylamino)-5-methyl-pyridazin-3-yl]-(4,5-dihydroxypentyl)amino]-5-[3-[4-[3-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-methyl-amino]prop-1-ynyl]-2-fluoro-phenoxy]propyl]thiazole-4-carboxylic acid

Using Method C and P46 as the appropriate payload, the desired product was obtained after a purification step based on the NH₄HCO₃ method (Prep-HPLC, general procedures). HRMS (ESI) [M+H]⁺=1324.4903 (δ=−1.7 ppm).

### Preparation of L9C-P17: 2-[3-(1,3-benzothiazol-2-ylamino)-4-methyl-6,7-dihydro-5H-pyrido[2,3-c]pyridazin-8-yl]-5-[3-[4-[3-[4-[[4-[[(2S)-2-[[(2S)-2-[3-[2-(2,5- dioxopyrrol-1-yl)ethoxy]propanoylamino]-3-methyl-butanoyl]amino]-5-ureido-pentanoyl]amino]phenyl]methoxycarbonyl]piperazin-1-yl]prop-1-ynyl]-2-fluoro-phenoxy]propyl]thiazole-4-carboxylic acid

Using Method C and P17 as the appropriate payload, the desired product was obtained. HRMS (ESI) [M+H]⁺ found=1299.4880 (δ=0.5 ppm).

### Preparation of L9A-P11: [3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]-1-methyl-prop-2-ynyl]-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-dimethyl-ammonium;2,2,2-trifluoroacetic acid

Using Method A and P11 as the appropriate payload, the desired product was obtained. HRMS (ESI) [M+H]⁺ found=1202.4722 (δ=0.5 ppm).

### Preparation of L9A-P8: 2-[3-(1,3-benzothiazol-2-ylamino)-4-methyl-6,7-dihydro-5H-pyrido[2,3-c]pyridazin-8-yl]-5-[3-[4-[3-[4-[[4-[[(2S)-2-[[(2S)-2-[3-[2-(2,5- dioxopyrrol-1-yl)ethoxy]propanoylamino]-3-methyl-butanoyl]amino]-5-ureido-pentanoyl]amino]phenyl]methyl]-4-methyl-piperazin-4-ium-1-yl]but-1-ynyl]-2-fluoro-phenoxy]propyl]thiazole-4-carboxylic acid;2,2,2-trifluoroacetic acid

Using Method D and P8 as the appropriate payload, the desired product was obtained. HRMS (ESI) [M+H]⁺ found=1 284.5343 (δ=−1.9 ppm).

### Preparation of L9A-P14: 2-[3-(1,3-benzothiazol-2-ylamino)-4-methyl-6,7-dihydro-5H-pyrido[2,3-c]pyridazin-8-yl]-5-[3-[4-[3-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl-diethyl-ammonio]prop-1-ynyl]-2-fluoro-phenoxy]propyl]thiazole-4-carboxylate

Using Method D and P14 as the appropriate payload, the desired product was obtained after a purification step based on the NH₄HCO₃ method (Prep-HPLC, general procedures). HRMS (ESI) [M+H]⁺ found=1242.5021 (δ=−0.2 ppm).

### Preparation of L9A-P13: 2-[3-(1,3-benzothiazol-2-ylamino)-4-methyl-6,7-dihydro-5H-pyrido[2,3-c]pyridazin-8-yl]-5-[3-[4-[3-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl-ethyl-methyl-ammonio]prop-1-ynyl]-2-fluoro-phenoxy]propyl]thiazole-4-carboxylate

Using Method D and P13 as the appropriate payload, the desired product was obtained after a purification step based on the NH₄HCO₃ method (Prep-HPLC, general procedures). HRMS (ESI) [M+H]⁺ found=1228.4855 (δ=−1.0 ppm).

### Preparation of L9A-P34: 3-[4-[3-[2-[3-(1,3-benzothiazol-2-ylamino)-4-methyl-6,7-dihydro-5H-pyrido[2,3-c]pyridazin-8-yl]-4-carboxy-thiazol-5-yl]propoxy]-3-fluoro-phenyl]prop-2-ynyl-[(3S)-3,4-dihydroxybutyl]-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-methyl-ammonium;2,2,2-trifluoroacetic acid

Using Method D and P34 as the appropriate payload, the desired product was obtained. HRMS (ESI) [M−CF₃CO₂]⁺ found=1288.5086 (δ=0.6 ppm).

### Method F

### Preparation of L13A-P2: [4-[[(2S)-2-[[(2S)-2-[3-[2-[2-[2-[2-[2-[2-[2-[2-[2-[2-[2-(2-azidoethoxy)ethoxy]ethoxy]ethoxy]ethoxy]ethoxy]ethoxy]ethoxy]ethoxy]ethoxy]ethoxy]e thoxy]propanoylamino]-3-methyl-butanoyl]amino]propanoyl]amino]phenyl]methyl-[3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl]-dimethyl-ammonium;2,2,2-trifluoroacetate

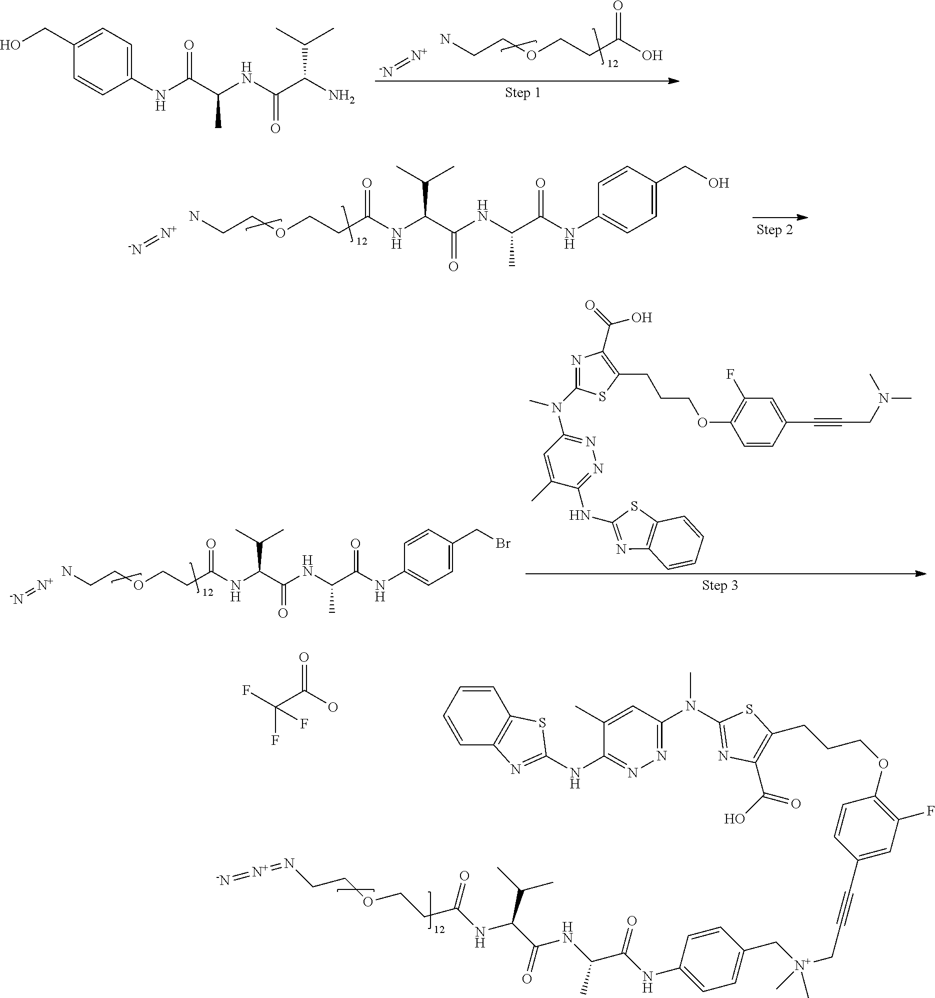

### Step 1: (2S)-2-[3-[2-[2-[2-[2-[2-[2-[2-[2-[2-[2-[2-(2-azidoethoxy)ethoxy]ethoxy]ethoxy]ethoxy]ethoxy]ethoxy]ethoxy]ethoxy]ethoxy]ethoxy]e thoxy]propanoylamino]-N-[(1S)-2-[4-(hydroxymethyl)anilino]-1-methyl-2-oxo-ethyl]-3-methyl-butanamide

To a suspension of (2S)-2-amino-N-[(1S)-2-[4-(hydroxymethyl)anilino]-1-methyl-2-oxo-ethyl]-3-methyl-butanamide (900 mg, 3.07 μmol) in DMF (10 mL) were successively added a solution of 3-[2-[2-[2-[2-[2-[2-[2-[2-[2-[2-[2-(2-azidoethoxy)ethoxy]ethoxy]ethoxy]ethoxy]ethoxy]ethoxy]ethoxy]ethoxy]ethoxy]ethoxy]ethoxy]pr opanoic acid (2.00 g, 3.07 mmol) in DMF (10 mL), EDC (650 mg, 3.38 mmol) as a powder and DIPEA (1.00 mL, 6.14 mmol). The reaction was stirred at room temperature for 16 h. The crude product was purified using C18 reverse phase prep-HPLC by direct deposit of the reaction mixture on the Xbridge® column and using the NH₄HCO₃ method to afford the desired product (1.64 g, 1.78 mmol). IR: (v cm−1) 3600-3200, 3287, 2106, 1668, 1630, 1100. ¹H NMR (400 MHz, dmso-d₆) δ ppm 9.82 (m, 1H), 8.14 (d, 1H), 7.87 (d, 1H), 7.54 (d, 2H), 7.23 (d, 2H), 5.08 (t, 1H), 4.43 (d, 2H), 4.39 (m, 1H), 4.20 (m, 1H), 3.65-3.44 (m, 48H), 3.39 (t, 2H), 2.5-2.3 (m, 2H), 1.97 (m, 1H), 1.31 (d, 3H), 0.87/0.84 (2d, 6H). HRMS (ESI) [M+H]⁺ found: 919.5234 (δ=3.4 ppm).

### Step 2: (2S)-2-[3-[2-[2-[2-[2-[2-[2-[2-[2-[2-[2-[2-(2-azidoethoxy)ethoxy]ethoxy]ethoxy]ethoxy]ethoxy]ethoxy]ethoxy]ethoxy]ethoxy]ethoxy]e thoxy]propanoylamino]-N-[(1S)-2-[4-(bromomethyl)anilino]-1-methyl-2-oxo-ethyl]-3-methyl-butanamide

To a solution of the product from Step 1 (72 mg, 7.83 μmol) in THF (5 mL) was added at 0° C. a 1M solution of PBr₃ in THF (157 μL, 157 μmol) and the reaction mixture was stirred for 1 h at 0° C. and for 1 h at room temperature. The reaction mixture was diluted with AcOEt (5 mL), treated with an aqueous saturated solution of NaHCO₃ (0.5 mL), dried over MgSO₄, and used without further treatment in the next step. IR: (v cm⁻¹) 3700-3100, 1658, 2106. HRMS (ESI) [M+H]⁺ found: 981.4390 (δ=1.3 ppm).

### Step 3: [4-[[(2S)-2-[[(2S)-2-[3-[2-[2-[2-[2-[2-[2-[2-[2-[2-[2-[2-(2-azidoethoxy)ethoxy]ethoxy]ethoxy]ethoxy]ethoxy]ethoxy]ethoxy]ethoxy]ethoxy]ethoxy]e thoxy]propanoylamino]-3-methyl-butanoyl]amino]propanoyl]amino]phenyl]methyl-[3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl]-dimethyl-ammonium;2,2,2-trifluoroacetate

To a solution of the product from Step 2 ((21 mg, 2.09 μmol) in DMF (2 mL) were successively added 2-[[6-(1,3-benzothiazol-2-ylamino)-5-methyl-pyridazin-3-yl]-methyl-amino]-5-[3-[4-[3-(dimethylamino)prop-1-ynyl]-2-fluoro-phenoxy]propyl]thiazole-4-carboxylic acid (P2) (11.0 mg, 1.74 μmol) as a powder and DIPEA (8.6 μL, 5.22 μmol). The reaction was stirred at room temperature for 8 h. The crude product was purified using C18 reverse phase prep-HPLC by direct deposit of the reaction mixture on the Xbridge® column and using the TFA method to afford the desired product (15 mg, 0.91 mmol). IR: (v cm⁻¹) 3400-3150, 2235, 2105, 1667. ¹H NMR (500 MHz, dmso-d₆) δ ppm 7.90 (dl, 1H), 7.76 (d, 2H), 7.68 (s, 1H), 7.58 (dd, 1H), 7.51 (m, 1H), 7.51 (d, 2H), 7.41 (m, 1H), 7.38 (t, 1H), 7.25 (m, 1H), 7.20 (t, 1H), 4.55 (s, 2H), 4.42 (s, 2H), 4.39 (m, 1H), 4.21 (m, 1H), 4.19 (t, 2H), 3.77 (s, 3H), 3.60 (m, 4H), 3.54/3.50 (m+m, 44H), 3.38 (t, 2H), 3.29 (m, 2H), 3.05 (s, 6H), 2.47 (s, 3H), 2.46/2.38 (m+m, 1+1H), 2.16 (quint, 2H), 1.96 (m, 1H), 1.32 (d, 3H), 0.88/0.84 (d+d, 3+3H). ¹³C NMR (500 MHz, dmso-d₆) δ ppm 133.9, 129.7, 126.4, 122.6, 122.1, 120.0, 119.3, 118.1, 115.3, 70.5/70.1, 70.1/67.5, 68.7, 66.2, 57.8, 53.7, 50.6, 49.7, 49.5, 36.4, 35.3, 31.0, 30.9, 23.3, 19.5/18.6, 18.4, 17.7. ¹⁹F NMR (500 MHz, dmso-d₆) δ ppm −133.8. HRMS (ESI) [M+H]⁺ found: 1532.6964 (δ=0.6 ppm).

### Method G

### Preparation of L19C-P7: 5-[3-[4-[3-[[4-[[(2S)-2-[[(2S)-2-[[2-[2-[2-(2-azidoethoxy)ethoxy]ethoxy]acetyl]amino]-3-methyl-butanoyl]amino]propanoyl]amino]phenyl]methoxycarbonyl-methyl-amino]prop-1-ynyl]-2-fluoro-phenoxy]propyl]-2-[[6-(1,3-benzothiazol-2-ylamino)-5-methyl-pyridazin-3-yl]-methyl-amino]thiazole-4-carboxylic acid

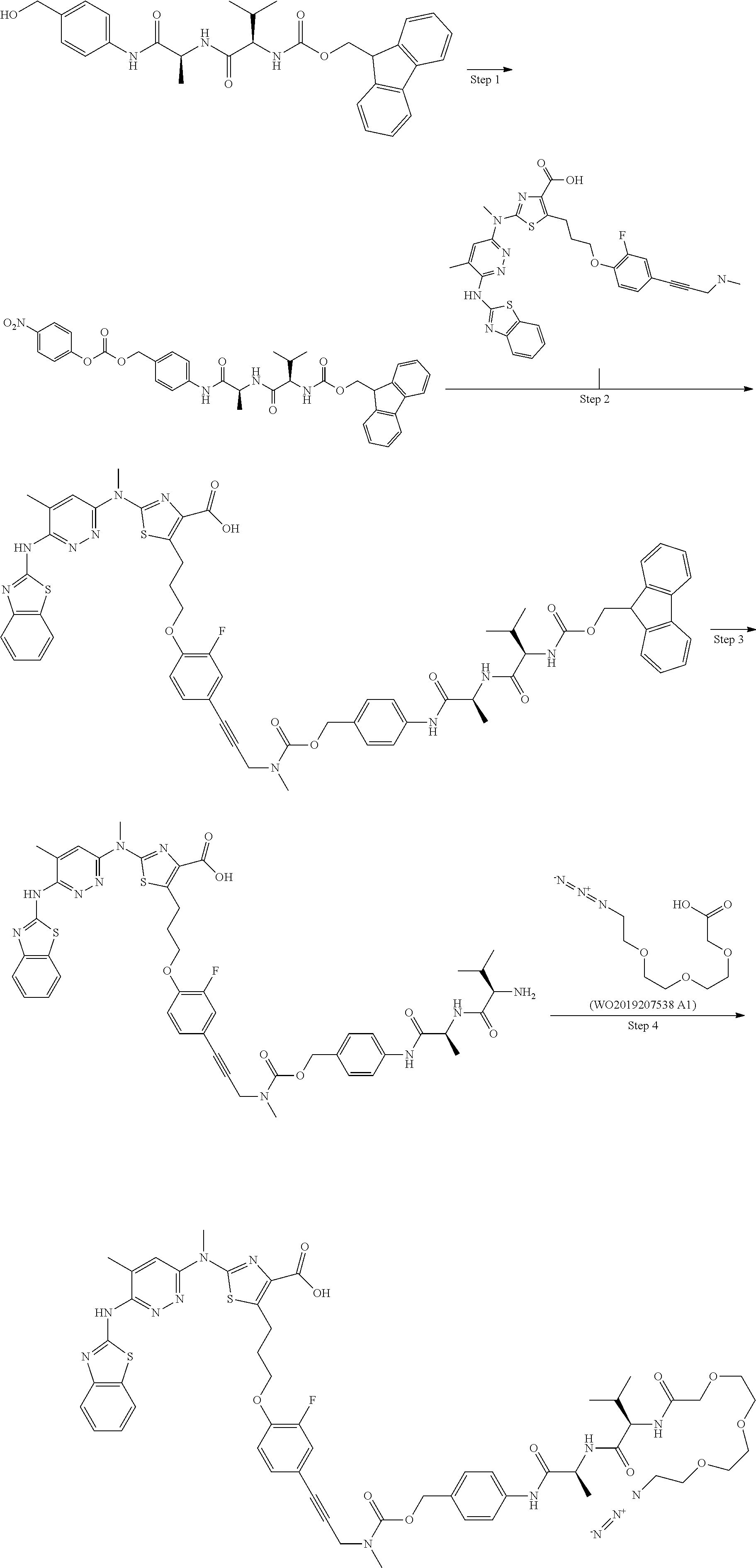

### Step 1: [4-[[(2S)-2-[[(2S)-2-(9H-fluoren-9-ylmethoxycarbonylamino)-3-methyl-butanoyl]amino]propanoyl]amino]phenyl]methyl (4-nitrophenyl) carbonate

To a solution of 9H-fluoren-9-ylmethyl N-[(1S)-1-[[(1S)-2-[4-(hydroxymethyl)anilino]-1-methyl-2-oxo-ethyl]carbamoyl]-2-methyl-propyl]carbamate (5.0 g, 9.7 mmol) in THF (20 mL) and DCM (10 mL) were successively added paranitrophenyl chlorocarbonate (4.1 g, 20.1 mmol) and pyridine (1.65 mL, 20.4 mmol). The reaction was stirred at room temperature for 15 h. A 10% aqueous solution of citric acid was added and the reaction mixture was extracted twice with AcOEt. The organic layer was washed with brine and dried over MgSO₄. After evaporation under vacuum the solid was dissolved in a minimum amount of AcOEt and ether was added to precipitate the desired compound (5.6 g, 8.22 mmol). IR: (v cm⁻¹) 3350-3200, 1760;1690;1670;1630, 1523;1290. ¹H NMR (400 MHz, dmso-d₆) δ ppm 10.07 (m, 1H), 8.31 (d, 2H), 8.19 (d, 1H), 7.89 (d, 2H), 7.74 (t, 2H), 7.64 (d, 2H), 7.57 (d, 2H), 7.41 (m, 2H), 7.41 (d, 2H), 7.4 (m, 1H), 7.32 (t, 2H), 5.24 (s, 2H), 4.43 (m, 1H), 4.36-4.19 (m, 3H), 3.92 (dd, 1H), 2 (m, 1H), 1.32 (d, 3H), 0.9/0.87 (2d, 6H).

### Step 2: 2-[[6-(1,3-benzothiazol-2-ylamino)-5-methyl-pyridazin-3-yl]-methyl-amino]-5-[3-[4-[3-[[4-[[(2S)-2-[[(2S)-2-(9H-fluoren-9-ylmethoxycarbonylamino)-3-methyl-butanoyl]amino]propanoyl]amino]phenyl]methoxycarbonyl-methyl-amino]prop-1-ynyl]-2-fluoro-phenoxy]propyl]thiazole-4-carboxylic acid

To a solution of 2-[[6-(1,3-benzothiazol-2-ylamino)-5-methyl-pyridazin-3-yl]-methyl-amino]-5-[3-[2-fluoro-4-[3-(methylamino)prop-1-ynyl]phenoxy]propyl]thiazole-4-carboxylic acid (P7)(366.0 mg, 559 mmol) in DMF (10 mL) were successively added the product from Step 1 (378 mg, 556 mmol) and DIPEA (368 μL, 2.22 mmol). The reaction mixture was stirred at room temperature for 16 h and then evaporated to dryness. The crude product was purified by silica gel chromatography (gradient of methanol in DCM) to afford the desired compound (15.6 mg, 9.64 μmol).

### Step 3: 5-[3-[4-[3-[[4-[[(2S)-2-[[(2S)-2-amino-3-methyl-butanoyl]amino]propanoyl]amino]phenyl]methoxycarbonyl-methyl-amino]prop-1-ynyl]-2-fluoro-phenoxy]propyl]-2-[[6-(1,3-benzothiazol-2-ylamino)-5-methyl-pyridazin-3-yl]-methyl-amino]thiazole-4-carboxylic acid

To a solution of the product from Step 2 (424 mg, 366 mmol) in DMF (4 mL) was added piperidine (90 μL, 914 mmol) and the reaction mixture was stirred at room temperature for 1 h. After evaporation to dryness, the crude product was purified by silica gel chromatography (gradient of methanol containing 2% NH₄OH in DCM) to afford the desired compound. IR: (v cm⁻¹) 3270, 3100-2400, 1680, 1520. ¹H NMR (400 MHz, dmso-d₆) δ ppm 10.58/10.2 (2*s, 1H), 8.55/8.28 (2*s, 1H), 7.9 (d, 1H), 7.65 (s, 1H), 7.62 (d, 2H), 7.52 (d, 1H), 7.39 (m, 1H), 7.35-7 (massif, 3H), 7.32 (d, 2H), 7.2 (m, 1H), 5.05 (s, 2H), 4.48 (m, 1H), 4.26 (s, 2H), 4.15 (t, 2H), 3.71 (s, 3H), 3.3 (t, 2H), 3.03 (d, 1H), 2.9 (s, 3H), 2.45 (s, 3H), 2.11 (quint, 2H), 1.91 (m, 1 H), 1.4-0.7 (br s, 2H), 1.32 (d, 3H), 0.88/0.78 (2*d, 6H). ¹⁹F NMR (400 MHz, dmso-d₆) δ ppm-134.

### Step 4:5-[3-[4-[3-[[4-[[(2S)-2-[[(2S)-2-[[2-[2-[2-(2-azidoethoxy)ethoxy]ethoxy]acetyl]amino]-3-methyl-butanoyl]amino]propanoyl]amino]phenyl]methoxycarbonyl-methyl-amino]prop-1-ynyl]-2-fluoro-phenoxy]propyl]-2-[[6-(1,3-benzothiazol-2-ylamino)-5-methyl-pyridazin-3-yl]-methyl-amino]thiazole-4-carboxylic acid

To a solution of 2-[2-[2-(2-azidoethoxy)ethoxy]ethoxy]acetic acid (58 mg, 249 μmol) in DMF (1 mL) were successively added TSTU (77 mg, 255 μmol) and DIPEA (190 μL, 1.12 mmol), and the reaction mixture was stirred at room temperature for 2 h. After the addition of the product from Step 3 (84 mg, 89.6 mmol) in DMF (1.5 mL), the reaction mixture was stirred at room temperature for 2 h. The crude product was purified using C18 reverse phase prep-HPLC by direct deposit of the reaction mixture on the Xbridge® column and using the NH₄HCO₃ to afford the desired compound (64 mg, 55.5 mmol). IR: (v cm⁻¹) 3700-2700, 2104, 1693/1656, 1227/1127. ¹H NMR (400 MHz, dmso-d₆) δ ppm 9.76 (s, 1H), 8.16 (dl, 1H), 7.83 (d, 1H), 7.62 (s, 1H), 7.56 (d, 2H), 7.51 (d, 1H), 7.36 (d, 1H), 7.35 (t, 1H), 7.29 (d, 2H), 7.24-7.08 (m, 3H), 7.18 (t, 1H), 5.04 (s, 2H), 4.44 (hept, 1H), 4.28 (dd, 1H), 4.26 (s, 2H), 4.16 (t, 2H), 3.94 (s, 2H), 3.75 (s, 3H), 3.58 (m, 10H), 3.35 (t, 2H), 3.27 (t, 2H), 2.91 (s, 3H), 2.45 (s, 3H), 2.13 (quint, 2H), 2.05 (m, 1H), 1.32 (d, 3H), 0.89/0.84 (2d, 6H). ¹⁹F NMR (400 MHz, dmso-d₆) δ ppm −133.9. HRMS ESI [M+H]⁺ found 1152.4207 (δ=1.5 ppm).

### Preparation of L23C-P7: 5-[3-[4-[3-[[4-[[(2S)-2-[[(2S)-2-[[2-[2-[2-(2-azidoethoxy)ethoxy]ethoxy]acetyl]amino]-3-methyl-butanoyl]amino]-5-ureido-pentanoyl]amino]phenyl]methoxycarbonyl-methyl-amino]prop-1-ynyl]-2-fluoro-phenoxy]propyl]-2-[[6-(1,3-benzothiazol-2-ylamino)-5-methyl-pyridazin-3-yl]-methyl-amino]thiazole-4-carboxylic acid

Product was obtained according to Method G by replacing 9H-fluoren-9-ylmethyl N-[(1S)-1-[[(1S)-2-[4-(hydroxymethyl)anilino]-1-methyl-2-oxo-ethyl]carbamoyl]-2-methyl-propyl]carbamate with 9H-fluoren-9-ylmethyl N-[(1S)-1-[[(1S)-1-[[4-(hydroxymethyl)phenyl]carbamoyl]-4-ureido-butyl]carbamoyl]-2-methyl-propyl]carbamate. IR: (v cm⁻¹) 3687-3060, 2104, very broad-1656, 1606, 1515, 754 and 725. ¹H NMR (400 MHz, dmso-d₆) δ ppm 7.90 (d, 1H), 7.70 (br s, 1H), 7.60 (d, 2H), 7.50 (m, 2H), 7.40 (t, 1H), 7.30 (d+m, 3 H), 7.20 (t, 1H), 7.15 (dd, 1H), 5.45 (m, 2H), 4.40 (m, 1H), 4.30 (m, 1H), 4.25 (s, 2H), 4.15 (t, 2H), 3.95 (s, 2H), 3.80 (s, 3H), 3.60/3.30 (2m, 12H), 3.30 (m, 2H), 3.00 (2m, 2H), 2.90 (s, 3 H), 2.45 (s, 3H), 2.15 (quint, 2H), 2.00 (m, 1H), 1.70/1.60 (2m, 2H), 1.45/1.4 (2m, 2H), 0.90/0.80 (2d, 6H). ¹⁹F NMR (400 MHz, dmso-d₆) δ ppm −134.2. HRMS ESI [M+H]⁺ found 1238.4675 (δ=0.4 ppm).

### Preparation of L110C-P7: 5-[3-[4-[3-[[4-[[(2S)-2-[[(2S)-2-[3-[2-[2-[2-[2- [2-[2-[2-[2-[2-[2-[2-(2-azidoethoxy)ethoxy]ethoxy]ethoxy]ethoxy]ethoxy]ethoxy]ethoxy]ethoxy]ethoxy]ethoxy]e thoxy]propanoylamino]-3-methyl-butanoyl]amino]-5-ureido-pentanoyl]amino]phenyl]methoxycarbonyl-methyl-amino]prop-1-ynyl]-2-fluoro-phenoxy]propyl]-2-[[6-(1,3-benzothiazol-2-ylamino)-5-methyl-pyridazin-3-yl]-methyl-amino]thiazole-4-carboxylic acid

Product was obtained according to Method G by replacing 9H-fluoren-9-ylmethyl N-[(1S)-1-[[(1S)-2-[4-(hydroxymethyl)anilino]-1-methyl-2-oxo-ethyl]carbamoyl]-2-methyl-propyl]carbamate with 9H-fluoren-9-ylmethyl N-[(1S)-1-[[(1S)-1-[[4-(hydroxymethyl)phenyl]carbamoyl]-4-ureido-butyl]carbamoyl]-2-methyl-propyl]carbamate and 2-[2-[2-(2-azidoethoxy)ethoxy]ethoxy]acetic acid with 3-[2-[2-[2-[2-[2-[2-[2-[2-[2-[2-[2-(2-azidoethoxy)ethoxy]ethoxy]ethoxy]ethoxy]ethoxy]ethoxy]ethoxy]ethoxy]ethoxy]ethoxy]ethoxy]propanoic acid. IR: (v cm⁻¹) 3560-3063, 2100, very broad-1651, 1608, 1514, 756 and 725. ¹H NMR (400 MHz, dmso-d₆) δ ppm 7.9 (d, 1H), 7.7 (br s, 1H), 7.6 (d, 2H), 7.5 (m, 1H), 7.4 (t, 1H), 7.4-7.1 (m, 3H), 7.3 (d, 2H), 7.2 (t, 1H), 5.4 (m, 2H), 4.4 (m, 1H), 4.3 (s, 2H), 4.25 (m, 1H), 4.15 (t, 2H), 3.8 (s, 3H), 3.65-3.4 (m, 50H), 3.3 (m, 2H), 3 (2m, 2H), 2.9 (s, 3 H), 2.45 (s, 3H), 2.4 (m, 2H), 2.1 (quint, 2H), 2 (m, 1H), 1.7/1.6 (2m, 2H), 1.4 (2m, 2H), 0.85 (2d, 6H). ¹⁹F NMR (400 MHz, dmso-d₆) d ppm −134.4. HRMS (ESI) [M+H]⁺ found 1648.7209 (δ=1.4 ppm).

### Method H

### Preparation of L27C-P3: 2-[3-(1,3-benzothiazol-2-ylamino)-4-methyl-6,7-dihydro-5H-pyrido[2,3-c]pyridazin-8-yl]-5-[3-[4-[3-[[4-[[(2S)-2-[[(2S)-2-[3-[2-(2,5-dioxopyrrol-1-yl)ethoxy]propanoylamino]-3-methyl-butanimidoyl]amino]-5-ureido-pentanoyl]amino]-2-sulfo-phenyl]methoxycarbonyl-methyl-amino]prop-1-ynyl]-2-fluoro-phenoxy]propyl]thiazole-4-carboxylic acid

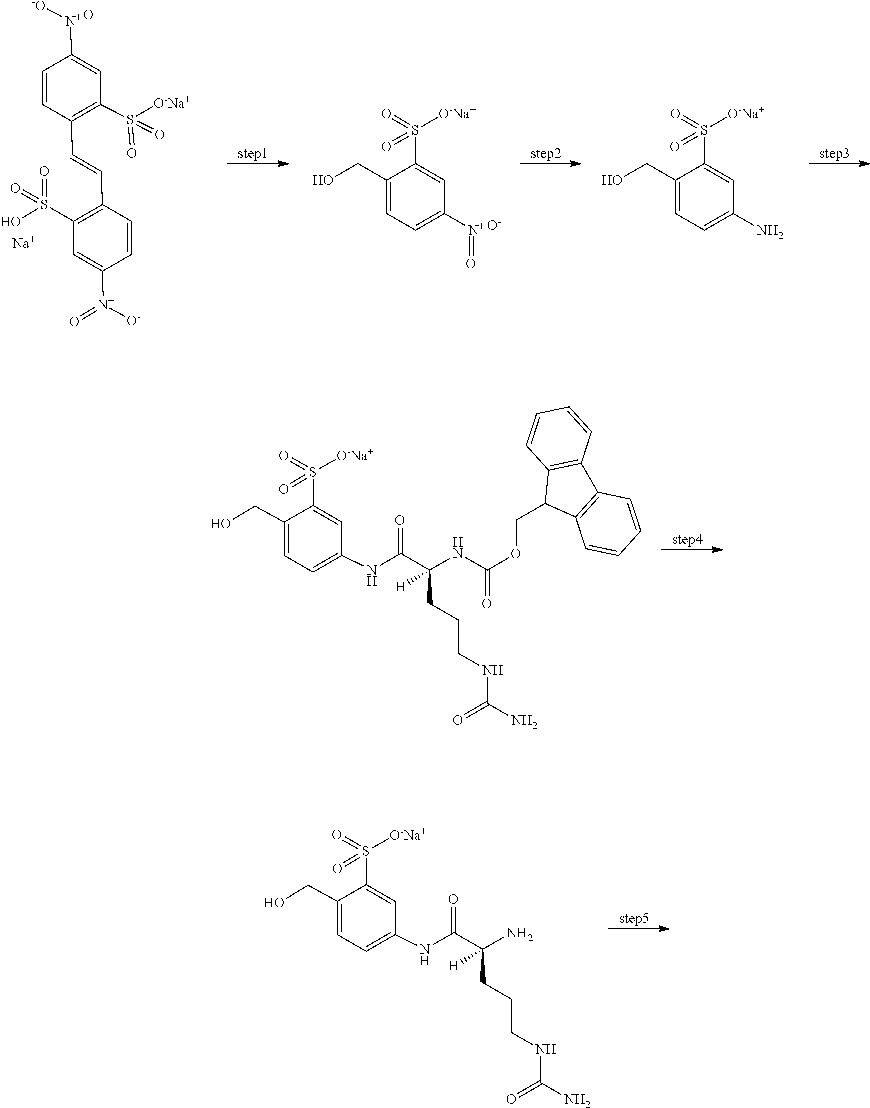

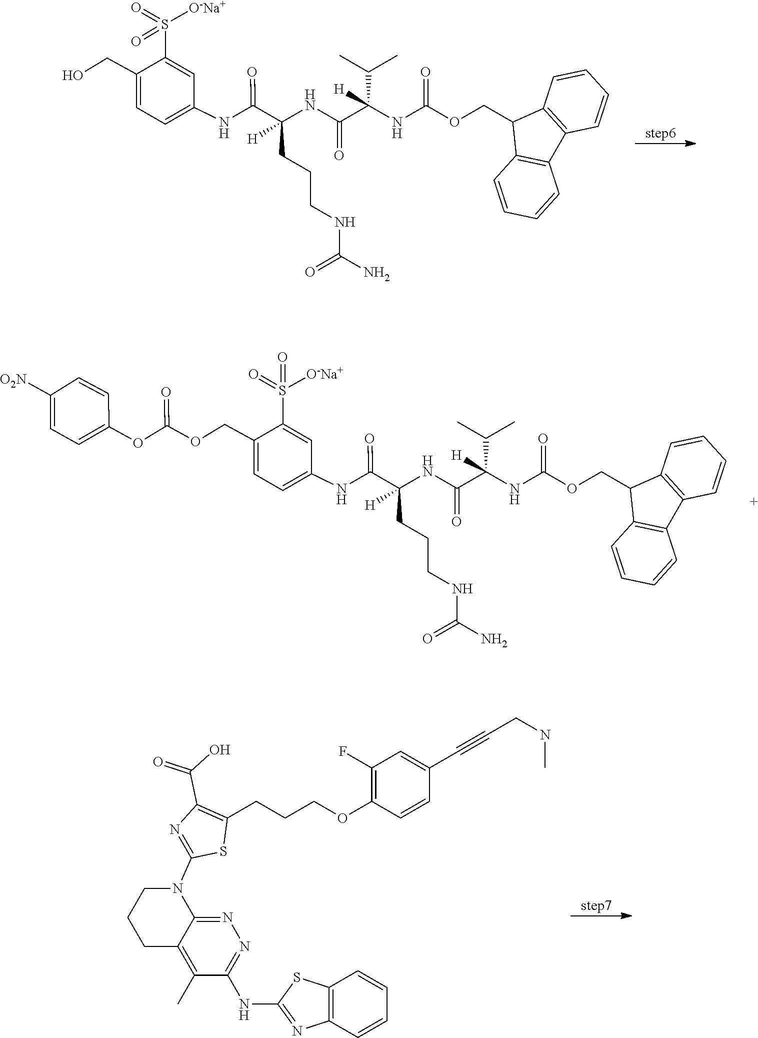

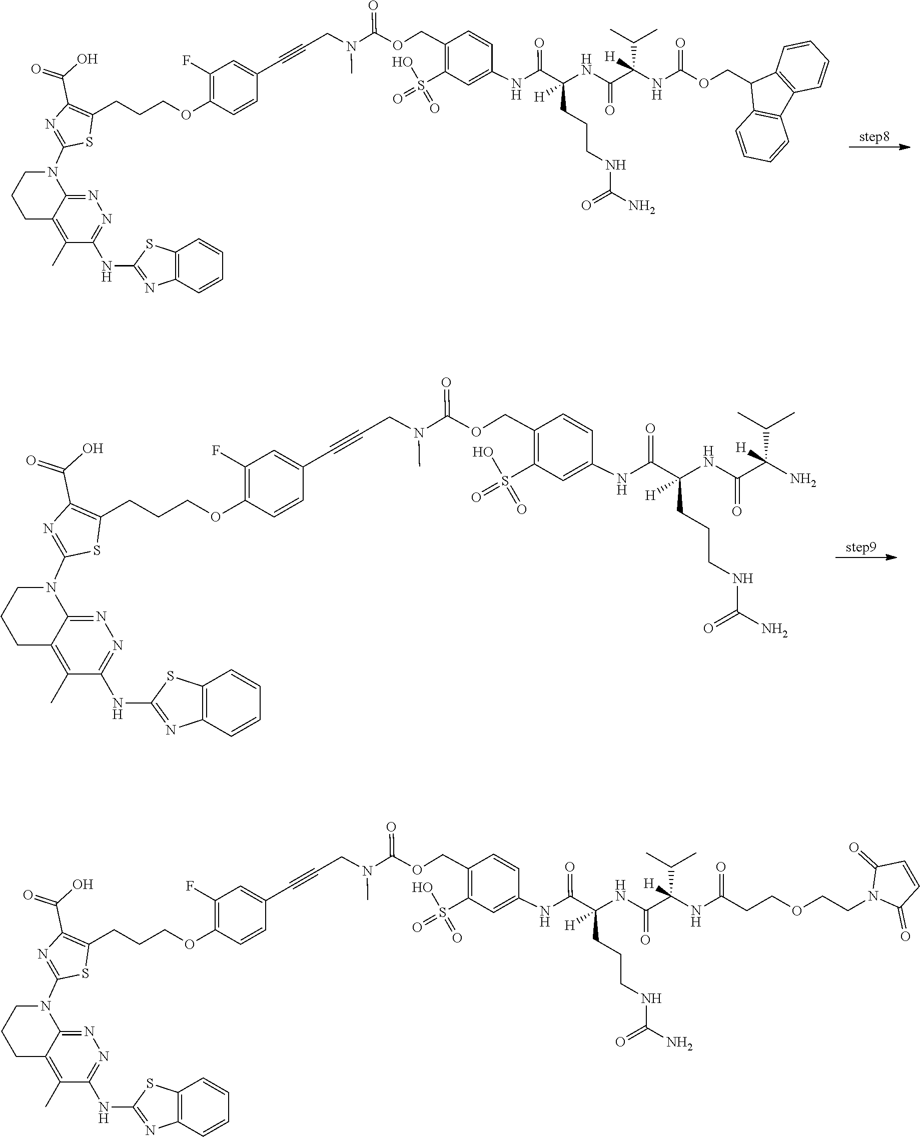 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)OCC4c5ccccc5-c5ccccc54)C(C)C)cc3S(=O)(=O)O)cc2F)s1 ; Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](N)C(C)C)cc3S(=O)(=O)O)cc2F)s1 ; Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3S(=O)(=O)O)cc2F)s1]

### Step 1: sodium;2-(hydroxymethyl)-5-nitro-benzenesulfonate

To a solution of sodium 5-nitro-2-[(E)-2-(4-nitro-2-sulfo-phenyl) vinyl]benzenesulfonate (25.0 g; 52.7 mmol) in water (336 mL) was introduced a stream of ozone for 1.5 h. After the completion of the reaction, the mixture was purged with argon for 30 minutes in order to remove the excess of ozone. Then, sodium carbonate (39.1 g; 7 eq.) and sodium borohydride (3.99 g; 2 eq.) were added and the orange solution was stirred at room temperature for 16 h. The reaction mixture was concentrated to give the desired compound (39.9 g; sup 100%) as a solid (containing residual traces of bore salts). ¹H NMR (dmso): δ 4.99 (d, 2H, J=3.6 Hz), 5.36 (t, 1H, J=5.6 Hz), 7.83 (d, 1H, J=8.4 Hz), 8.21 (d, 1H, J=8.4 Hz), 8.45 (s, 1H).

### Step 2: sodium;5-amino-2-(hydroxymethyl)benzenesulfonate

Sodium 2-(hydroxymethyl)-5-nitro-benzenesulfonate (26.9 g; 105 mmol) was solubilized in water (403 mL). Then, the reaction mixture was flushed with argon. Palladium 10% on carbon (2.65 g, 10% wt.) was added then the black suspension was flushed with argon and then with hydrogen. The reaction mixture was stirred at room temperature for 3.5 days under hydrogen atmosphere. After filtration over Celite® and washing with water and methanol, the filtrate was concentrated to dryness and co-evaporated 3 times with toluene. Purification by column chromatography on silica gel using ethyl acetate/methanol (90/10 to 70/30) as eluent afforded the desired compound (14.29 g; 60%). ¹H NMR (dmso): δ 4.52 (d, 2H, J=5.2 Hz), 4.95 (t, 1H, J=5.2 Hz), 5.04 (s, 2H), 6.42 (d, 1H, J=7.6 Hz), 6.93 (d, 1H, J=7.6 Hz), 7.03 (s, 1H).

### Step 3: sodium;5-[[(2S)-2-(9H-fluoren-9-ylmethoxycarbonylamino)-5-ureido-pentanoyl]amino]-2-(hydroxymethyl)benzenesulfonate

To a solution of Fmoc-L-Cit-OH (882 mg; 2.22 mmol) in dimethylformamide (32.5 mL) was added the product from Step 2 (500 mg; 2.22 mmol), HBTU (1.01 g; 2.66 mmol) and DIPEA (917 μL; 5.55 mmol). The reaction mixture was stirred at room temperature for 16 hours, then was concentrated to dryness and co-evaporated with water (2×100 mL). The crude was purified by column chromatography on C18 using acetonitrile/water 2/8 to 8/2 as eluent, to afford the desired compound (1.0 g; 63%). ¹H NMR (dmso): δ 1.25-1.28 (m, 15H, DIPEA), 1.36-1.72 (m, 4H), 2.92-3.03 (m, 2H), 3.11-3.18 (m, 2H, DIPEA), 3.5-3.65 (m, 2H, DIPEA), 4.30-4.12 (m, 4H), 4.74 (d, 2H, J=4.4 Hz), 5.05 (t, 1H, J=5.6 Hz), 5.37 (s, 2H), 5.97 (t, 1H, J=4.8 Hz), 7.34-7.42 (m, 4H), 7.62-7.90 (m, 7H), 8.15 (s, 1H), 10.05 (s, 1H).

### Step 4: sodium;5-[[(2S)-2-amino-5-ureido-pentanoyl]amino]-2-(hydroxymethyl)benzenesulfonate

To a solution of the product from Step 3 (11.2 g; 15.73 mmol) in DMF (224 mL) was added piperidine (3.1 mL; 2 eq.). The reaction mixture was stirred at room temperature for 3 hours then water (400 mL) was added. The aqueous layer was extracted with ethyl acetate (2×300 mL) and with dichloromethane (300 mL). Sodium carbonate (5.01 g;3 eq.) was added to the aqueous layer and the mixture was stirred at room temperature for 3 h. The mixture was lyophilized in order to give the desired compound (6.01 g; estimated to 100%) as a solid contaminated by sodium salts. ¹H NMR (dmso): δ 1.55-1.64 (m, 4H), 2.99-3.01 (m, 2H), 3.58 (m, 1H), 4.75 (s, 2H), 5.06 (s, 1H), 5.38 (s, 2H), 5.98 (t, 1H, J=5.6 Hz), 7.38 (d, 1H, J=8.4 Hz), 7.72 (dd, 1H, J=8.4 & 2.4 Hz), 7.86 (d, 1H, J=2.4 Hz,), 10.17 (s, 1H).

### Step 5: sodium; 5-[[(2S)-2-[[(2S)-2-(9H-fluoren-9-ylmethoxycarbonylamino)-3-methyl-butanoyl]amino]-5-ureido-pentanoyl]amino]-2-(hydroxymethyl)benzenesulfonate

To a solution of the product from Step 4 (6.01 g, 15.73 mmol) in dimethylformamide (150 mL) was added Fmoc-L-Val-OSu (6.85 g, 1 eq.). The solution was stirred at room temperature for 3 hours then the reaction mixture was diluted with saturated sodium hydrogenocarbonate (100 mL) and water (100 mL) and concentrated to dryness. The residue was purified on silica gel using ethyl acetate/methanol 90/10 to 50/50 as eluent to afford the desired compound (4.44 g, 48%). ¹H NMR (dmso): 0.85-0.90 (m, 6H), 1.31-1.76 (m, 4H), 1.95-2.06 (m, 1H), 2.91-3.05 (m, 2H), 3.95 (t, 1H, J=8.4 Hz), 4.24-4.35 (m, 3H), 4.37-4.45 (m, 1H), 4.76 (d, 2H, J=6 Hz), 5.07 (t, 1H, J=6.4 Hz,), 5.40 (s, 2H), 6.03 (t, 1H, J=5.6 Hz), 7.32-7.46 (m, 6H), 7.67 (d, 1H, J=8 Hz), 7.76 (t, 2H, J=7.2 Hz), 7.88-7.91 (m, 3H), 8.12 (d, 1H, J=7.6 Hz), 10.08 (s, 1H). 13C NMR (dmso): 18.25, 19.24, 26.70, 29.56, 30.45, 39.50, 46.67, 53.17, 60.01, 60.96, 65.66, 117.85, 119.15, 120.05, 125.36, 127.06, 127.62, 128.09, 134.39, 136.79, 140.67, 143.89, 145.34, 156.08, 158.82, 170.37, 171.16. LCMS (2-100 ACN/H₂O+0.1% AF): 93.85% retention time=8.4 min, Positive mode: 682.15 detected (MH⁺), Negative mode: 680.17 detected (MH).

### Step 6:5-[[(2S)-2-[[(2S)-2-(9H-fluoren-9-ylmethoxycarbonylamino)-3-methyl-butanoyl]amino]-5-ureido-pentanoyl]amino]-2-[(4-nitrophenoxy)carbonyloxymethyl]benzenesulfonate

To a solution of the product from Step 5 (450 mg, 0.64 mmol) in DMF (6 mL) was added DIPEA (1.34 mL, 7.67 mmol) and bis(4-nitrophenyl) carbonate (778 mg, 2.56 mmol). The solution was stirred at room temperature for 2 h and bis(4-nitrophenyl) carbonate (390 mg, 1.28 mmol) was added. After 1 h, the solution was concentrated under reduced pressure and the residue was purified by silica gel chromatography (gradient of methanol and acetic acid in dichloromethane) to give the desired compound (523 mg).

### Step 7:2-[3-(1,3-benzothiazol-2-ylamino)-4-methyl-6,7-dihydro-5H-pyrido[2,3-c]pyridazin-8-yl]-5-[3-[4-[3-[[4-[[(2S)-2-[[(2S)-2-(9H-fluoren-9-ylmethoxycarbonylamino)-3-methyl-butanimidoyl]amino]-5-ureido-pentanoyl]amino]-2-sulfo-phenyl]methoxycarbonyl-methyl-amino]prop-1-ynyl]-2-fluoro-phenoxy]propyl]thiazole-4-carboxylic acid

To a solution of 2-[3-(1,3-benzothiazol-2-ylamino)-4-methyl-6,7-dihydro-5H-pyrido[2,3-c]pyridazin-8-yl]-5-[3-[2-fluoro-4-[3-(methylamino)prop-1-ynyl]phenoxy]propyl]thiazole-4-carboxylic acid (P3)(70 mg, 109 μmol) in DMF (550 μL) were successively added DIPEA (0.19 mL, 1.39 mmol), the product of Step 6 (111 mg, 131 μmol) and DIEPA (95 μL, 544 μmol). The solution was stirred at room temperature for 15 h and concentrated to give the desired compound, which was used without any further treatment.

### Step 8:5-[3-[4-[3-[[4-[[(2S)-2-[[(2S)-2-amino-3-methyl-butanimidoyl]amino]-5-ureido-pentanoyl]amino]-2-sulfo-phenyl]methoxycarbonyl-methyl-amino]prop-1-ynyl]-2-fluoro-phenoxy]propyl]-2-[3-(1,3-benzothiazol-2-ylamino)-4-methyl-6,7-dihydro-5H-pyrido[2,3-c]pyridazin-8-yl]thiazole-4-carboxylic acid

To a solution of the product from Step 7 (147 mg, 109 μmol) in dioxane (1.1 mL) was added a solution of LiOH×H₂O (13.7 mg, 326 μmol) in water (1.1 mL). The solution was stirred at room temperature for 12 h. A 1 M aqueous solution of HCl was added until pH 7. The reaction mixture was evaporated to dryness and the residue triturated in DCM. The precipitate was washed with water and EtOH to give the desired compound (120 mg).

### Step 9:2-[3-(1,3-benzothiazol-2-ylamino)-4-methyl-6,7-dihydro-5H-pyrido[2,3-c]pyridazin-8-yl]-5-[3-[4-[3-[[4-[[(2S)-2-[[(2S)-2-[3-[2-(2,5-dioxopyrrol-1-yl)ethoxy]propanoylamino]-3-methyl-butanimidoyl]amino]-5-ureido-pentanoyl]amino]-2-sulfo-phenyl]methoxycarbonyl-methyl-amino]prop-1-ynyl]-2-fluoro-phenoxy]propyl]thiazole-4-carboxylic acid

To a solution of the product from Step 8 (120 mg, 109 μL) were successively added (2,5-dioxopyrrolidin-1-yl) 3-[2-(2,5-dioxopyrrol-1-yl)ethoxy]propanoate (37.8 mg, 122 μmol) and DIPEA (38.5 μL, 221 μmol). The solution was stirred at room temperature for 1.5 h. The crude product was purified using C18 reverse phase prep-HPLC by direct deposit of the reaction mixture on the Xbridge® column and using the TFA method to afford the desired compound (9 mg). HRMS (ESI) [M+H]⁺1322.3831 (δ=−3.3 ppm).

### Method I

### Preparation of L27A-P1: 3-[4-[3-[2-[3-(1,3-benzothiazol-2-ylamino)-4-methyl-6,7-dihydro-5H-pyrido[2,3-c]pyridazin-8-yl]-4-carboxy-thiazol-5-yl]propoxy]-3-fluoro-phenyl]prop-2-ynyl-[[4-[[(2S)-2-[[(2S)-2-[3-[2-(2,5-dioxopyrrol-1-yl)ethoxy]propanoylamino]-3-methyl-butanimidoyl]amino]-5-ureido-pentanoyl]amino]-2-sulfo-phenyl]methyl]-dimethyl-ammonium;2,2,2-trifluoroacetate

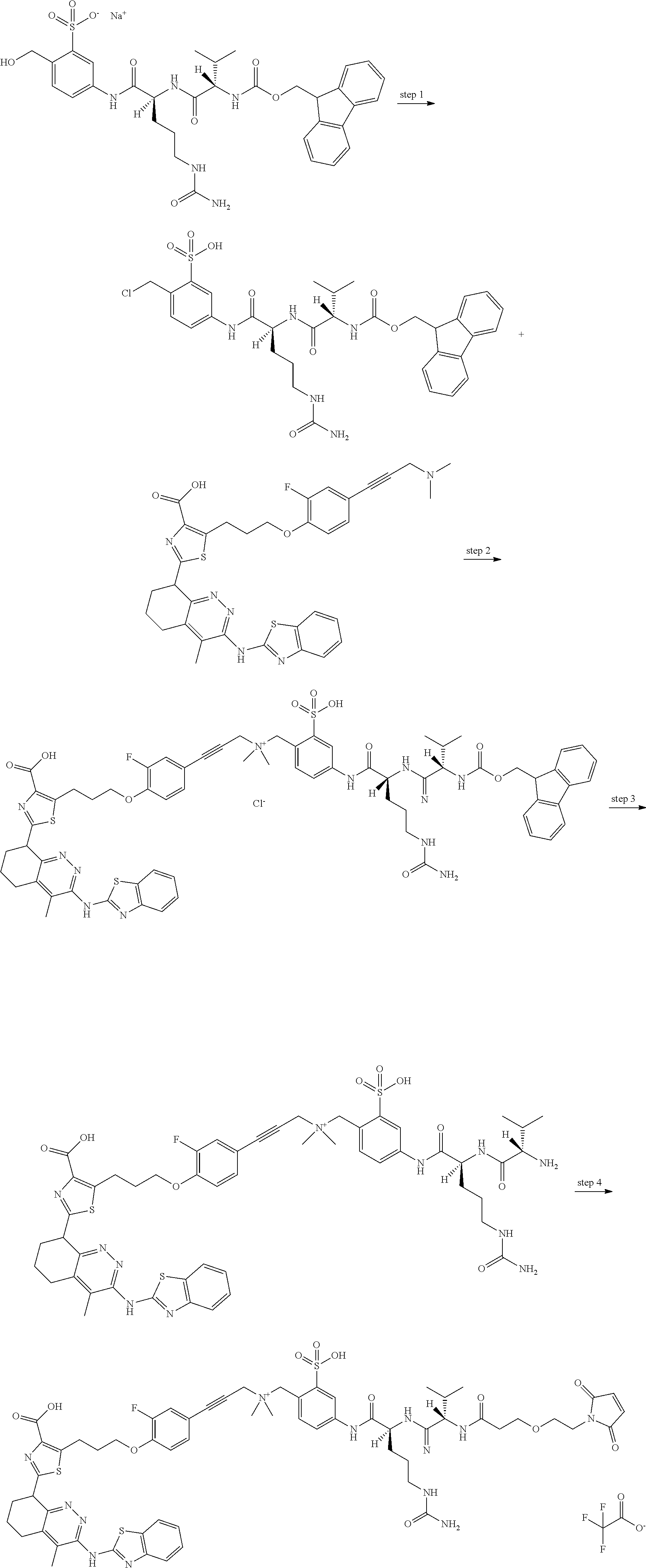

### Step 1:2-(chloromethyl)-5-[[(2S)-2-[[(2S)-2-(9H-fluoren-9-ylmethoxycarbonylamino)-3-methyl-butanoyl]amino]-5-ureido-pentanoyl]amino]benzenesulfonic acid

To a solution of 5-[[(2S)-2-[[(2S)-2-(9H-fluoren-9-ylmethoxycarbonylamino)-3-methyl-butanoyl]amino]-5-ureido-pentanoyl]amino]-2-(hydroxymethyl)benzenesulfonate (300 mg, 426 μmol) in NMP (6 mL) was added 7 times over 1 h a solution of SOCl₂ (31 μL, 426 μmol) in NMP (1 mL). The reaction mixture was stirred at room temperature for 1 h. The product was purified using C18 reverse phase prep-HPLC by direct deposit of the reaction mixture on the Oasis column and using the TFA method to give the desired product (225 mg). IR: (v cm-1) 3600-2200, 1657, 1250-1100. ¹H NMR (400 MHz, dmso-d₆) δ ppm 10.15/8.1/7.42/6 (s+2d+m, 4H), 7.9 (m, 3H), 7.75 (m, 3H), 7.42/7.31 (2m, 5H), 5.23 (s, 2H), 4.4 (m, 1H), 4.3-4.2 (m, 3H), 3.95 (dd, 1 H), 3 (m, 2H), 2 (m, 1H), 1.7/1.6 (2m, 2H), 1.48/1.37 (2m, 2H), 0.88 (2d, 6H). HRMS (ESI) [M+H]⁺700.2199 (δ=−0.5 ppm).

### Step 2:3-[4-[3-[2-[3-(1,3-benzothiazol-2-ylamino)-4-methyl-6,7-dihydro-5H-pyrido[2,3-c]pyridazin-8-yl]-4-carboxy-thiazol-5-yl]propoxy]-3-fluoro-phenyl]prop-2-ynyl-[[4-[[(2S)-2-[[(2S)-2-(9H-fluoren-9-ylmethoxycarbonylamino)-3-methyl-butanimidoyl]amino]-5-ureido-pentanoyl]amino]-2-sulfo-phenyl]methyl]-dimethyl-ammonium;chloride

To a solution of the product from Step 1 (55.7 mg, 68.4 μmol) in NMP (0.9 mL) were successively added 2-[3-(1,3-benzothiazol-2-ylamino)-4-methyl-6,7-dihydro-5H-pyrido[2,3-c]pyridazin-8-yl]-5-[3-[4-[3-(dimethylamino)prop-1-ynyl]-2-fluoro-phenoxy]propyl]thiazole-4-carboxylic acid (P1)(30 mg, 45.6 μmol), DIEPA (63.6 μL, 365 μmol), and TBAl (13 mg, 36.5 μmol). The reaction mixture was stirred at 60° C. for 6 h. The desired compound was directly used as a solution in Step 3.

### Step 3: [4-[[(2S)-2-[[(2S)-2-amino-3-methyl-butanoyl]amino]-5-ureido-pentanoyl]amino]-2-sulfo-phenyl]methyl-[3-[4-[3-[2-[3-(1,3-benzothiazol-2-ylamino)-4-methyl-6,7-dihydro-5H-pyrido[2,3-c]pyridazin-8-yl]-4-carboxy-thiazol-5-yl]propoxy]-3-fluoro-phenyl]prop-2-ynyl]-dimethyl-ammonium

To the NMP solution of the product from Step 2 (26.5 μmol) was added diethylamine (21.9 μL, 212 μmol). The reaction mixture was stirred at room temperature for 24 h. The crude product was purified using C18 reverse phase prep-HPLC by direct deposit of the reaction mixture on the Oasis column and using the NH₄HCO₃ method to give the desired product (18 mg).

### Step 4:3-[4-[3-[2-[3-(1,3-benzothiazol-2-ylamino)-4-methyl-6,7-dihydro-5H-pyrido[2,3-c]pyridazin-8-yl]-4-carboxy-thiazol-5-yl]propoxy]-3-fluoro-phenyl]prop-2-ynyl-[[4-[[(2S)-2-[[(2S)-2-[3-[2-(2,5-dioxopyrrol-1-yl)ethoxy]propanoylamino]-3-methyl-butanimidoyl]amino]-5-ureido-pentanoyl]amino]-2-sulfo-phenyl]methyl]-dimethyl-ammonium;2,2,2-trifluoroacetate

To a solution of the product from Step 3 (20 mg, 18.2 μmol) in DMF (900 μL) were successively added (2,5-dioxopyrrolidin-1-yl) 3-[2-(2,5-dioxopyrrol-1-yl)ethoxy]propanoate (8.5 mg, 27.3 μmol) and DIPEA (9.5 μL, 54.5 μmol). The solution was stirred at room temperature for 1.5 h. The crude product was purified using C18 reverse phase prep-HPLC by direct deposit of the reaction mixture on the Xbridge® column and using the NH₄HCO₃ method to give the title compound (15.7 mg). HRMS (ESI) [M+H]⁺1294.4278 0=1 ppm.

### Method J

### Preparation of L21A-P2: 3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl-[[2-[2-[(2S,3R,4R,5S,6S)-6-carboxy-3,4,5-trihydroxy-tetrahydropyran-2-yl]ethyl]-4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-dimethyl-ammonium;2,2,2-trifluoroacetate

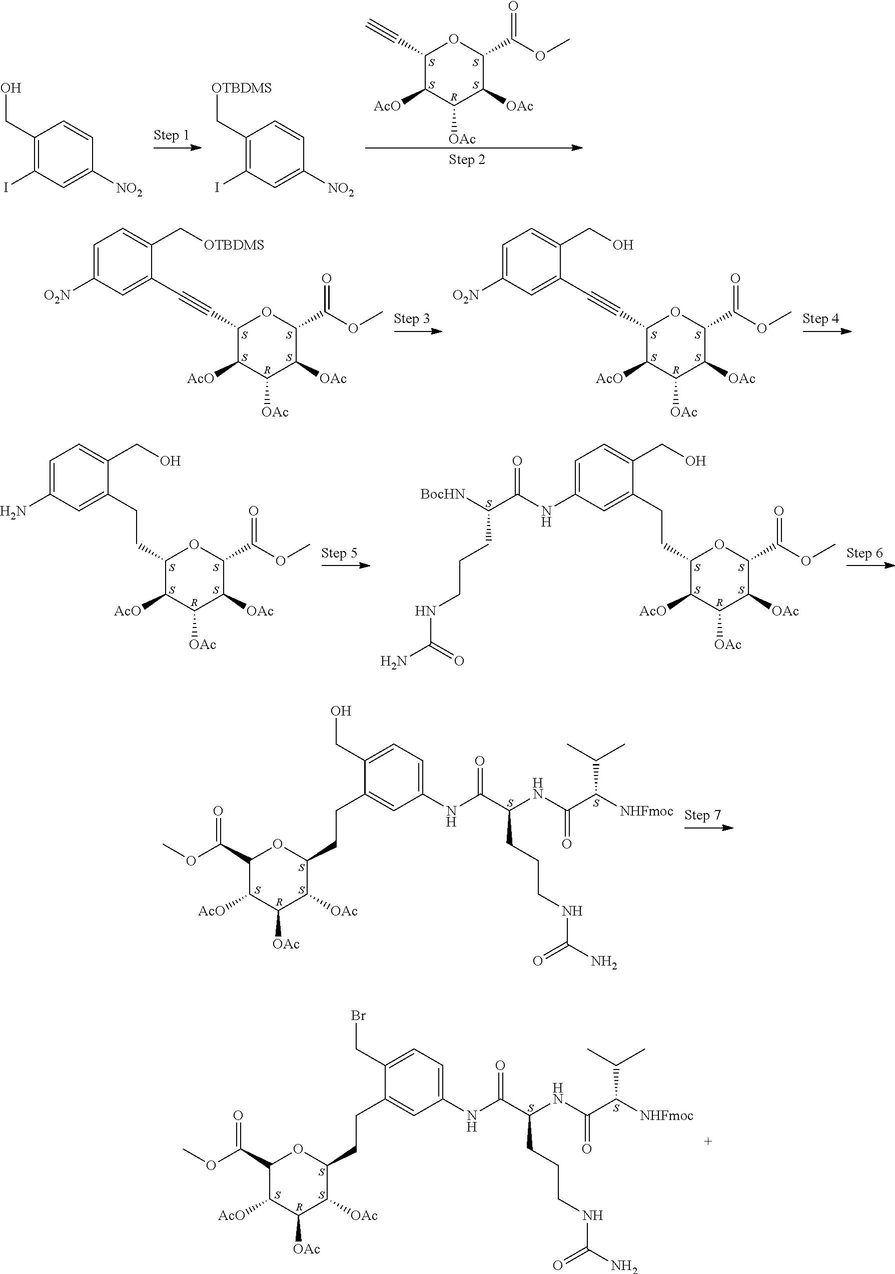

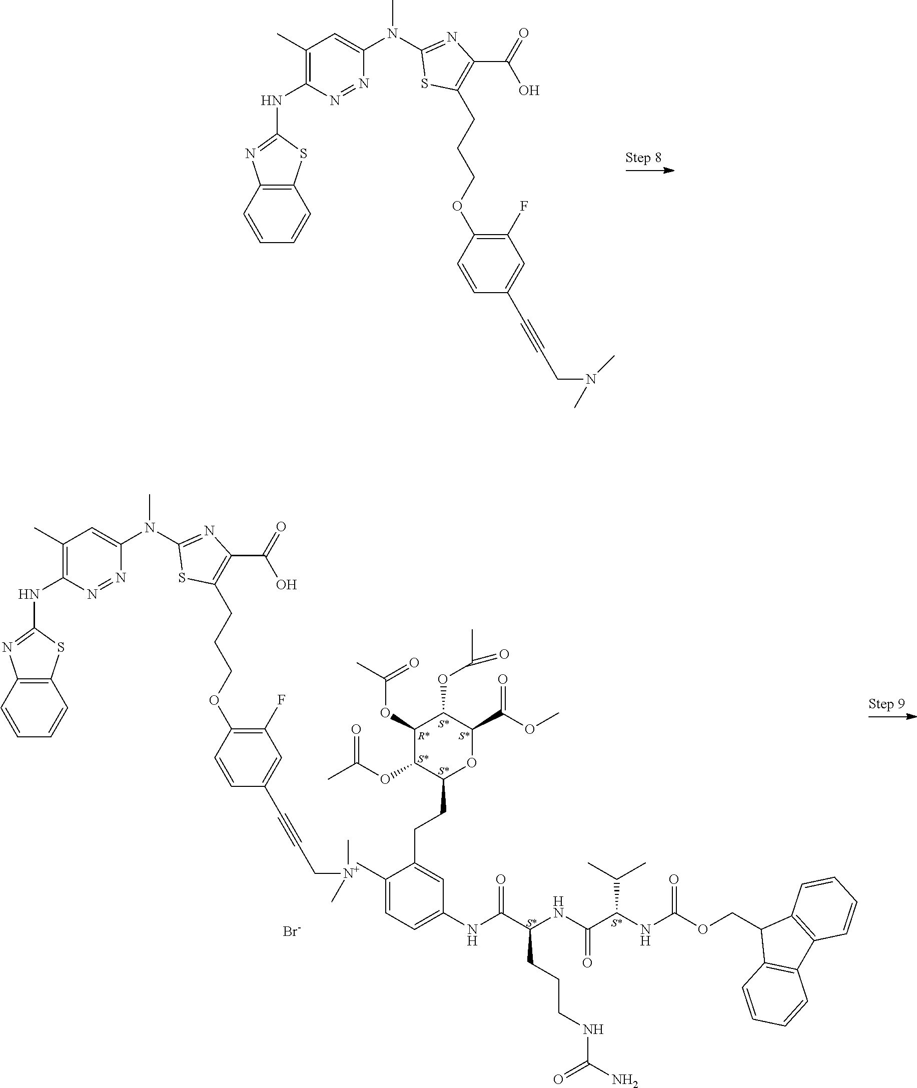

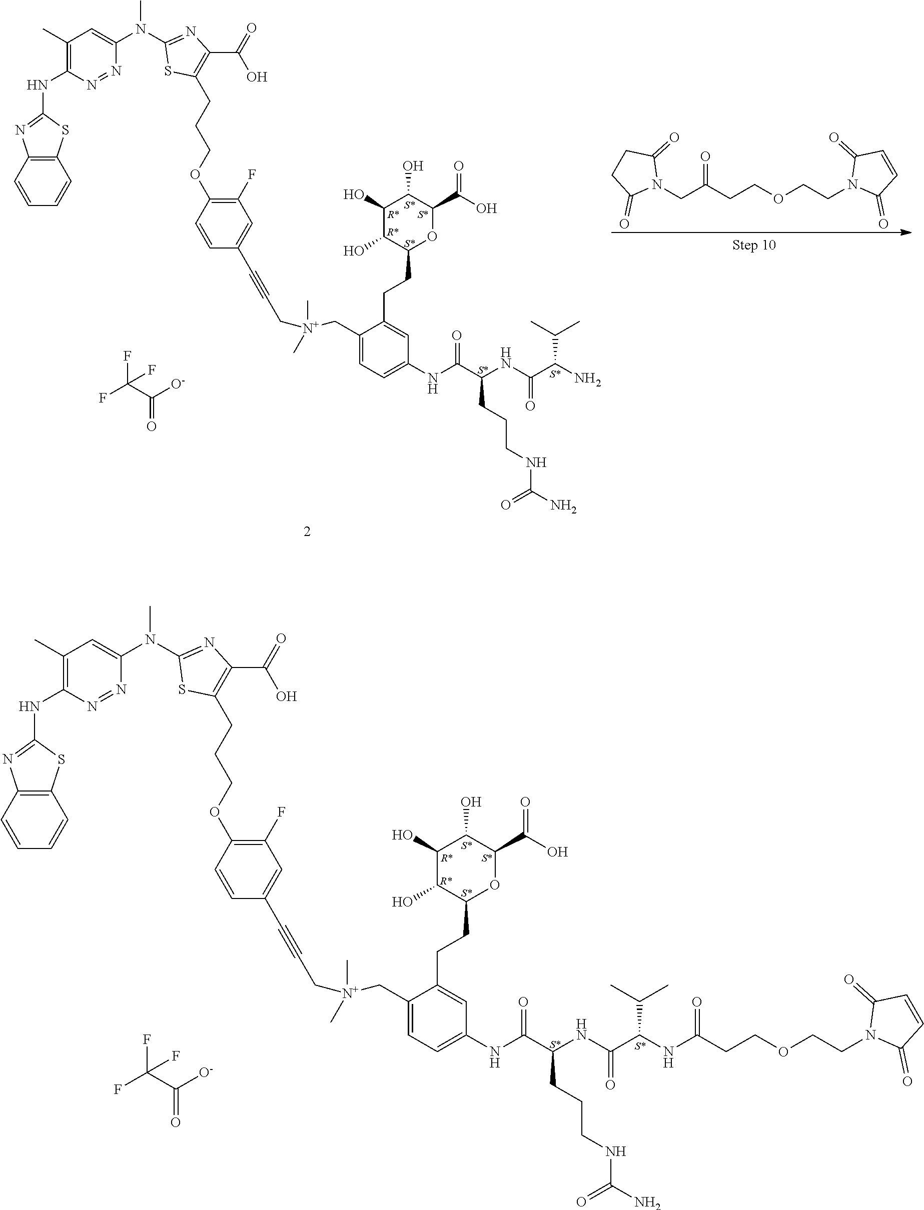

### Step 1: tert-butyl-[(2-iodo-4-nitro-phenyl)methoxy]-dimethyl-silane

To a solution of (2-iodo-4-nitro-phenyl)methanol (172 g, 61.64 mmol) in dichloromethane (300 mL) was added imidazole (5.04 g, 73.97 mmol). After the mixture was cooled to 0° C., a solution of tert-butyl-chloro-dimethyl-silane (TBDMSCl)(11.15 g, 73.97 mmol) in dichloromethane (300 mL) was added dropwise in 15 min. After stirring at room temperature for 16 h, the reaction mixture was quenched with methanol (20 mL) and concentrated to dryness. The crude product was purified by silica gel chromatography (gradient of ethyl acetate in cyclohexane) to give the desired product (19.65 g). ¹H NMR (400 MHz, dmso-d₆): δ 8.57 (s, 1H), 8.31 (d, 1H), 7.66 (d, 1H), 4.67 (s, 2H), 0.92 (s, 9H), 0.14 (s, 6H).

### Step 2: methyl (2S,3S,4R,5S,6S)-3,4,5-triacetoxy-6-[2-[2-[[tert-butyl(dimethyl)silyl]oxymethyl]-5-nitro-phenyl]ethynyl]tetrahydropyran-2-carboxylate

To a solution of the product from Step 1 (3.0 g, 7.63 mmol) in DMF (55 mL) were successively added methyl (2S,3S,4R,5S,6S)-3,4,5-triacetoxy-6-ethynyl-tetrahydropyran-2-carboxylate (3.39 g, 9.92 mmol), DIPEA (5.80 mL, 35.09 mmol), copper iodide (145 mg, 0.763 mmol) and dichloro-bis-(triphenylphosphine)palladium(II)(535 mg, 0.763 mmol). The solution was flushed with argon and stirred at room temperature for 16 h. After dilution with water (300 mL), the aqueous layer was extracted with ethyl acetate (2×300 mL). The combined organic layers were washed with water (2×300 mL), dried, filtered, and concentrated to dryness. The crude product was purified by silica gel chromatography (gradient of ethyl acetate in cyclohexane) to give the desired product (4.01 g). ¹H NMR (400 MHz, dmso-d₆): δ 8.32 (dd, 1H), 8.19 (d, 1H), 7.75 (d, 1H), 5.45 (t, 1H), 5.16 (t, 1H), 5.02-5.07 (m, 2H), 4.82 (s, 2H), 4.55 (d, 1H), 3.65 (s, 3H), 1.98-2.07 (m, 9H), 0.92 (m, 9H), 0.14 (s, 6H).

### Step 3: methyl (2S,3S,4R,5S,6S)-3,4,5-triacetoxy-6-[2-[2-(hydroxymethyl)-5-nitro-phenyl]ethynyl]tetrahydropyran-2-carboxylate

To a solution of the product from Step 2 (4.01 g, 6.60 mmol) in THF (48 mL) and water (48 mL) was added acetic acid (193 mL, 3.36 mol). The solution was stirred at room temperature for 2 days then diluted with water (300 mL). The aqueous layer was extracted with dichloromethane (2×300 mL). The combined organic layers were washed with water (2×300 mL) and with a saturated aqueous solution of sodium hydrogen carbonate (400 mL), dried, filtered, and concentrated to dryness. The crude product was purified by silica gel chromatography (gradient of ethyl acetate in cyclohexane) to give the desired product (2.67 g). ¹H NMR (400 MHz, dmso-d₆): δ 8.29 (dd, 1H), 8.15 (d, 1H), 7.79 (d, 1H), 5.68 (t, 1H), 5.45 (t, 1H), 5.16 (t, 1H), 5.02-5.07 (m, 2H), 4.62 (d, 2H), 4.55 (d, 1H), 3.65 (s, 3H), 1.98-2.07 (m, 9H).

### Step 4: methyl (2S,3S,4R,5S,6S)-3,4,5-triacetoxy-6-[2-[5-amino-2-(hydroxymethyl)phenyl]ethyl]tetrahydropyran-2-carboxylate

A solution of the product from Step 3 (2.67 g, 5.41 mmol) in THF (59 mL) was flushed with argon. After adding Platinum on carbon 5% dry (1.34 g, 50% w/w), the reaction mixture was successively flushed with argon and with H₂, then stirred under H₂ atmosphere (1 atm) at room temperature for 2 days. The reaction mixture was filtered through a Celite® pad, washed with a solution of ethyl acetate/methanol 9/1 (500 mL), and concentrated to dryness. All the sequence (including addition of platinum on carbon 5% dry (1.34 g, 50% w/w), stirring under H₂ (1 atm) at room temperature for 16 h and filtration through a Celite® pad) was repeated to allow the complete conversion. The crude product was purified by silica gel chromatography (gradient of ethyl acetate in cyclohexane) to give the desired product (1.12 g). ¹H NMR (400 MHz, dmso-d₆): δ 6.93 (d, 1H). 6.67-6.33 (m, 2H), 5.30 (t, 1H), 4.96 (t, 1H), 4.88 (s, 2H), 4.81 (t, 1H), 4.61 (t, 1H), 4.39 (d, 1H), 4.29-4.24 (m, 2H), 3.78-3.72 (m, 1H), 3.65 (s, 3H), 2.65-2.54 (m, 2H), 2.07-1.98 (m, 9H), 1.79-1.68 (m, 1H), 1.63-1.52 (m, 1H).

### Step 5: methyl (2S,3S,4R,5S,6S)-3,4,5-triacetoxy-6-[2-[5-[[(2S)-2-(tert-butoxycarbonyl amino)-5-ureido-pentanoyl]amino]-2-(hydroxymethyl)phenyl]ethyl]tetrahydropyran-2-carboxylate

To a solution of the product from Step 4 (1.00 g, 2.14 mmol) in DMF (21 mL) were successively added (2S)-2-(tert-butoxycarbonylamino)-5-ureido-pentanoic acid (Boc-Cit-OH) (589 mg, 2.14 mmol), DIPEA (707 μl, 4.28 mmol) and HBTU (1.22 g, 3.21 mmol). The reaction mixture was stirred at room temperature for 72 h. After dilution with water (100 mL) and concentration, the crude product was purified by silica gel chromatography (gradient of methanol in dichloromethane) to afford the desired product (1.05 g). ¹H NMR (400 MHz, dmso-d₆): δ 9.82 (s, 1H), 7.35-7.42 (m, 2H), 7.24 (d, 1H), 6.95 (d, 1H), 5.94 (t, 1H), 5.37 (s, 2H), 5.30 (t, 1H), 4.91-4.99 (m, 2H), 4.79 (t, 1H), 4.36-4.42 (m, 3H), 4.01-4.08 (m, 1H), 3.76 (t, 1H), 3.65 (s, 3H), 2.95-3.04 (m, 2H), 2.54-2.65 (m, 2H), 1.98-2.07 (m, 9H), 1.68-1.79 (m, 1H), 1.49-1.63 (m, 3H), 1.30-1.42 (m, 11H).

### Step 6: methyl (2S,3S,4R,5S,6S)-3,4,5-triacetoxy-6-[2-[5-[[(2S)-2-[[(2S)-2-(9H-fluoren-9-ylmethoxycarbonylamino)-3-methyl-butanoyl]amino]-5-ureido-pentanoyl]amino]-2-(hydroxymethyl)phenyl]ethyl]tetrahydropyran-2-carboxylate

To a solution of the product form Step 5 (950 mg, 1.31 mmol) in dichloromethane (7.5 mL) was added trifluoroacetic acid (1.9 mL, 25.6 mmol) at 0° C. The reaction mixture was stirred at room temperature for 3 h. The reaction mixture was concentrated to dryness and coevaporated with toluene (2×50 mL) to afford the crude compound. To this crude in solution in DMF (13 mL) were successively added (2S)-2-(9H-fluoren-9-ylmethoxycarbonylamino)-3-methyl-butanoic acid (Fmoc-Val-OH)(467 mg, 1.38 mmol), DIPEA (867 μl, 5.24 mmol) and HBTU (845 mg, 2.23 mmol). The reaction mixture was stirred at room temperature for 16 h. A saturated aqueous solution of hydrogenocarbonate (20 mL) was added and the mixture was stirred at room temperature for 1 h, diluted with water (100 mL) and concentrated to dryness. The crude product was purified by silica gel chromatography (gradient of methanol in dichloromethane) and then by reverse phase C18 chromatography using the neutral method to give the desired product (680 mg). LC-MS: MS (ESI) m/z [M+H]⁺=946.3. ¹H NMR (400 MHz, dmso-d₆): δ 9.90 (s, 1H). 8.07 (d, 2H), 7.89 (d, 2H), 7.74 (t, 2H), 7.44-7.38 (m, 3H), 7.36-7.28 (m, 3H), 7.24 (d, 1H), 5.94 (t, 1H), 5.37 (s, 2H), 5.30 (t, 1H), 4.99-4.92 (m, 2H), 4.79 (t, 1H), 4.42-4.36 (m, 4H), 4.32-4.19 (m, 3H), 3.94-3.90 (m, 1H), 3.76 (t, 1H), 3.65 (s, 3H), 2.99-2.94 (m, 2H), 2.65-2.54 (m, 2H), 2.07-1.98 (m, 10H), 1.70-1.55 (m, 4H), 1.46-1.36 (m, 2H), 0.89-0.84 (m, 6H). ¹³C NMR (100 MHz, dmso-d₆): δ 171.19, 170.33, 169.58, 169.45, 169.27, 167.77, 158.81, 156.12, 143.89, 143.76, 140.69, 139.48, 137.54, 134.88, 128.44, 127.62, 127.06, 125.35, 120.08, 119.42, 116.65, 75.78, 74.61, 72.65, 71.20, 69.49, 65.68, 60.49, 60.10, 53.14, 52.40, 46.68, 32.32, 30.43, 29.54, 27.19, 26.77, 20.39, 20.34, 20.24, 19.22, 18.25.

### Step 7: methyl (2S,3S,4R,5S,6S)-3,4,5-triacetoxy-6-[2-[2-(bromomethyl)-5-[[(2S)-2-[[(2S)-2-(9H-fluoren-9-ylmethoxycarbonylamino)-3-methyl-butanoyl]amino]-5-ureido-pentanoyl]amino]phenyl]ethyl]tetrahydropyran-2-carboxylate

To a solution of the product from Step 6 (154 mg, 0.163 mmol) in THF (8.2 mL) were successively added triphenylphosphine (85.4 mg, 0.326 mmol) and 1-bromopyrrolidine-2,5-dione (58.0 mg, 0.326 mmol). The reaction mixture was stirred at room temperature for 2 h. After 5 h, triphenylphosphine (85.4 mg, 0.326 mmol) and 1-bromopyrrolidine-2,5-dione (58.0 mg, 0.326 mmol) were added to the mixture and the reaction was stirred at room temperature for 15 h. The crude product thus obtained was used in the next step. UPLC-MS: MS (ESI) m/z [M+OMe−Br+H]⁺=960.7.

### Step 8:3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl-dimethyl-[[4-[[(2S)-2-[[(2S)-2-(9H-fluoren-9-ylmethoxycarbonylamino)-3-methyl-butanoyl]amino]-5-ureido-pentanoyl]amino]-2-[2-[(2S,3S,4R,5S,6S)-3,4,5-triacetoxy-6-methoxycarbonyl-tetrahydropyran-2-yl]ethyl]phenyl]methyl]ammonium; bromide

To the solution of the product from Step 7 (207.63 mg, 206 μmol) in DMF (5 mL) were successively added 2-[[6-(1,3-benzothiazol-2-ylamino)-5-methyl-pyridazin-3-yl]-methyl-amino]-5-[3-[4-[3-(dimethylamino)prop-1-ynyl]-2-fluoro-phenoxy]propyl]thiazole-4-carboxylic acid (P2) (100 mg, 158 μmol) and DIPEA (135 μL, 792 μmol). The reaction mixture was stirred at room temperature for 4 h. The crude product was concentrated and used in the next step without further treatment (246 mg).

### Step 9:3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl-dimethyl-[[2-[2-[(2S,3R,4R,5S,6S)-6-carboxy-3,4,5-trihydroxy-tetrahydropyran-2-yl]ethyl]-4-[[(2S)-2-[[(2S)-2-amino-3-methyl-butanoyl]amino]-5-ureido-pentanoyl]amino]phenyl]methyl]ammonium;2,2,2-trifluoroacetate;2,2,2-trifluoroacetic acid

To a solution of the product from Step 8 (246 mg, 158 μmol) in dioxane (2.0 mL) was added a solution of lithium hydroxide monohydrate (39.7 mg, 946 μmol) in water (2 ml). After the completion of the reaction, a 1 M aqueous solution of HCl was added until pH 6-7. The crude product was purified using C18 reverse phase prep-HPLC by direct deposit of the reaction mixture on the Xbridge® column and using the TFA method to afford the expected compound (68 mg).

### Step 10:3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl-dimethyl-[[2-[2-[(2S,3R,4R,5S,6S)-6-carboxy-3,4,5-trihydroxy-tetrahydropyran-2-yl]ethyl]-4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]ammonium;2,2,2-trifluoroacetate

To a solution of the product from Step 9 (30 mg, 21.0 μmol) in DMF (1.2 mL) were successively added the solution of (2,5-dioxopyrrolidin-1-yl) 3-[2-(2,5-dioxopyrrol-1-yl)ethoxy]propanoate (12.8 mg, 41.3 μmol) in DMF (500 μL) and DIPEA (18.3 μL, 105 μmol). The reaction mixture was stirred at room temperature for 3 h. The crude product was purified using C18 reverse phase prep-HPLC by direct deposit of the reaction mixture on the Xbridge® column and using the TFA method to afford the title compound (6.5 mg). HRMS (ESI) [M−CF₃COO]⁺ found=1392.5197 (δ=0.7 ppm).

### Preparation of L106A-P2: 3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl-[[2-[2-[(2S,3R,4R,5S,6S)-6-carboxy-3,4,5-trihydroxy-tetrahydropyran-2-yl]ethyl]-4-[[(2S)-2-[[(2S)-2-[3-[2-(2,5-dioxopyrrol-1-yl)ethoxy]propanoylamino]-3-methyl-butanoyl]amino]propanoyl]amino]phenyl]methyl]-dimethyl-ammonium;2,2,2-trifluoroacetate

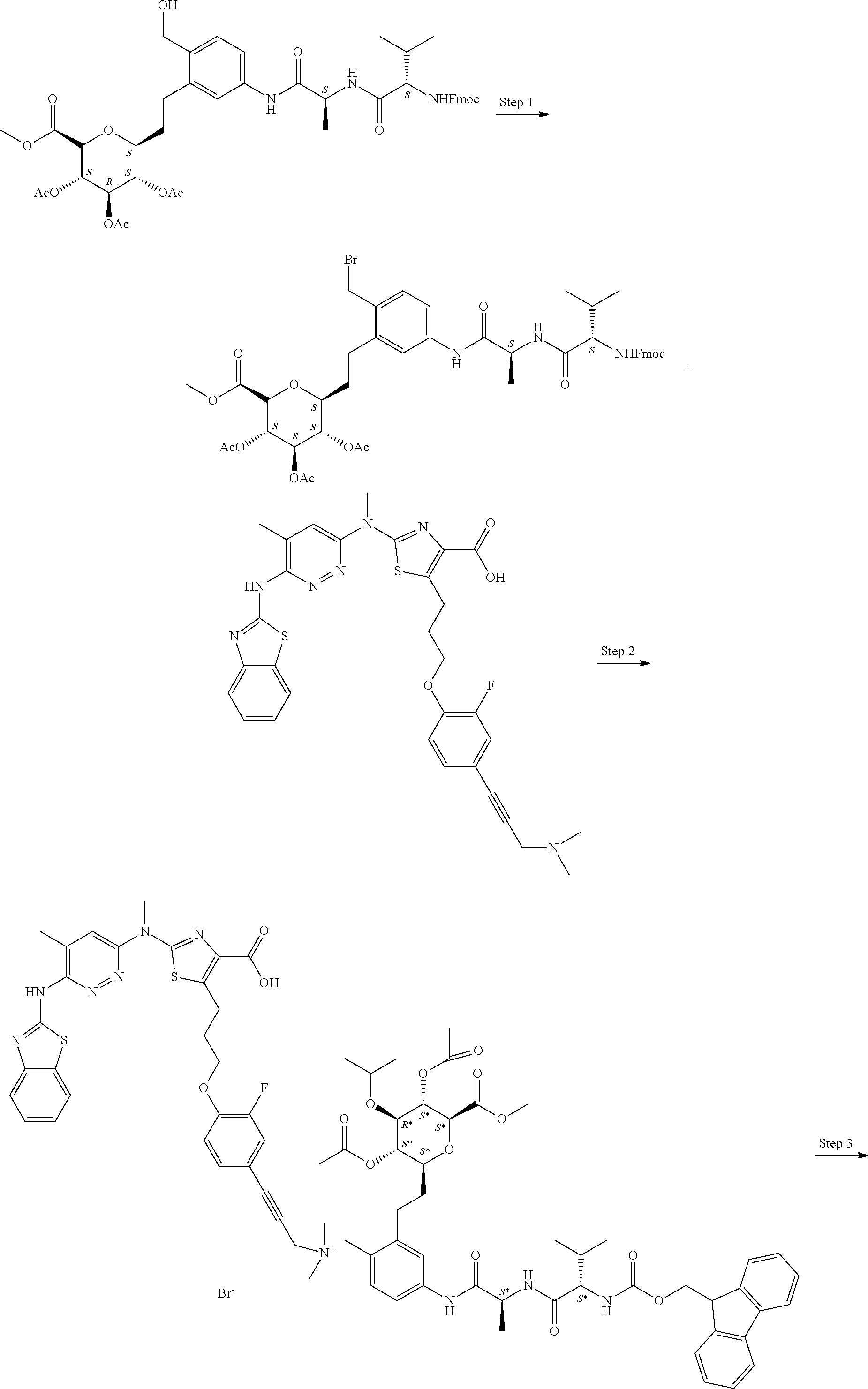

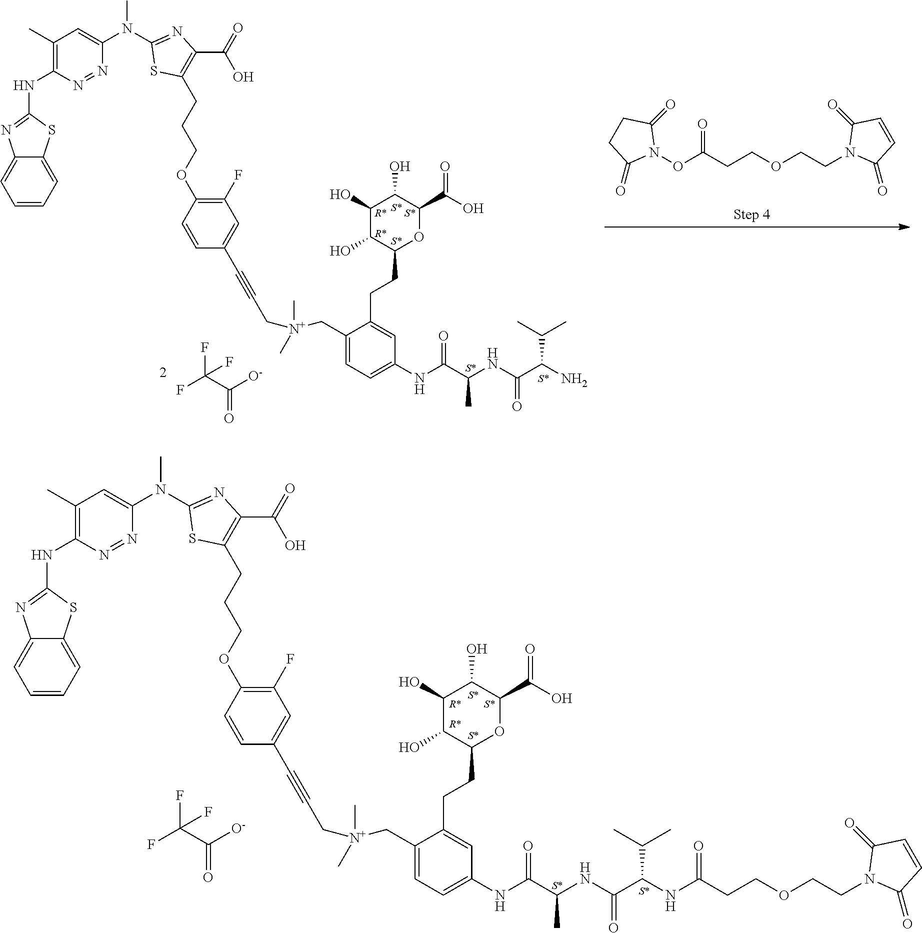 [SMILES: Cc1cc(N(C)c2nc(C(=O)O)c(CCCOc3ccc(C#CC[N+](C)(C)Cc4ccc(NC(=O)[C@H](C)NC(=O)[C@@H](N)C(C)C)cc4CC[C@@H]4O[C@H](C(=O)O)[C@@H](O)[C@H](O)[C@H]4O)cc3F)s2)nnc1Nc1nc2ccccc2s1 ; O=C([O-])C(F)(F)F ; O=C(CCOCCN1C(=O)C=CC1=O)ON1C(=O)CCC1=O ; Cc1cc(N(C)c2nc(C(=O)O)c(CCCOc3ccc(C#CC[N+](C)(C)Cc4ccc(NC(=O)[C@H](C)NC(=O)[C@@H](NC(=O)CCOCCN5C(=O)C=CC5=O)C(C)C)cc4CC[C@@H]4O[C@H](C(=O)O)[C@@H](O)[C@H](O)[C@H]4O)cc3F)s2)nnc1Nc1nc2ccccc2s1 ; O=C([O-])C(F)(F)F]

### Step 1: methyl (3S,4R,5S,6S)-3,4,5-triacetoxy-6-[2-[2-(bromomethyl)-5-[[(2S)-2-[[(2S)-2-(9H-fluoren-9-ylmethoxycarbonylamino)-3-methyl-butanoyl]amino]propanoyl]amino]phenyl]ethyl]tetrahydropyran-2-carboxylate

To a solution of methyl (3S,4R,5S,6S)-3,4,5-triacetoxy-6-[2-[5-[[(2S)-2-[[(2S)-2-(9H-fluoren-9-ylmethoxycarbonylamino)-3-methyl-butanoyl]amino]propanoyl]amino]-2-(hydroxymethyl)phenyl]ethyl]tetrahydropyran-2-carboxylate (Preparation of L106C-P7, Step 16)(255 mg, 297 μmol) in THF (14 mL) were successively added triphenylphosphine (234 mg, 890 μmol) and N-bromosuccinimide (158 mg, 890 μmol). The reaction mixture was stirred at room temperature for 15 h. The reaction mixture was used in the next step without any treatment.

### Step 2:3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl-dimethyl-[[4-[[(2S)-2-[[(2S)-2-(9H-fluoren-9-ylmethoxycarbonylamino)-3-methyl-butanoyl]amino]propanoyl]amino]-2-[2-[(2S,3S,4R,5S,6S)-3,4,5-triacetoxy-6-methoxycarbonyl-tetrahydropyran-2-yl]ethyl]phenyl]methyl]ammonium;bromide

To a suspension of the product from Step 1 (297 μmol) in THF were successively added a solution 2-[[6-(1,3-benzothiazol-2-ylamino)-5-methyl-pyridazin-3-yl]-methyl-amino]-5-[3-[4-[3-(dimethylamino)prop-1-ynyl]-2-fluoro-phenoxy]propyl]thiazole-4-carboxylic acid (P2)(140 mg, 222 μmol) in DMF (3 mL) and DIPEA (116 μL, 665 μmol). The reaction was stirred at room temperature for 60 h. The reaction mixture was evaporated to dryness and used without work-up in the next step.

### Step 3:3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl-dimethyl-[[2-[2-[(2S,3R,4R,5S,6S)-6-carboxy-3,4,5-trihydroxy-tetrahydropyran-2-yl]ethyl]-4-[[(2S)-2-[[(2S)-2-amino-3-methyl-butanoyl]amino]propanoyl]amino]phenyl]methyl]ammonium;2,2,2-trifluoroacetate;2,2,2-trifluoroacetic acid

To a solution of the product from Step 2 (222 μmol) in dioxane (2 mL) was added a solution of LiOH·H₂O (218 mg, 5.20 mmol) in water (2 mL). The solution was stirred at room temperature for 2 h. A 1 M aqueous solution of HCl was added until pH 6-7. The reaction mixture was evaporated to dryness and the crude product was purified using C18 reverse phase prep-HPLC by direct deposit of the reaction mixture on the Xbridge® column and using the TFA method to afford the expected compound (112 mg). IR: (v cm⁻¹) 3500-2500, 2237, 1667, 1197/1180/1130. ¹H NMR (400/500 MHz, dmso-d₆) δ ppm 12.55 (m), 10.35 (s), 8.65 (d), 8.1 (large), 7.89 (d, 1H), 7.67 (s, 1H), 7.66 (dd, 1H), 7.53 (df, 1H), 7.48 (m, 1H), 7.4 (m, 1H), 7.38 (m, 1H), 7.27 (m, 1H), 7.24 (t, 1H), 7.2 (dd, 1H), 7.19 (m, 1H), 5.3-4.7 (ml), 4.64/4.54 (2d, 2H), 4.51 (br s, 2H), 4.5 (m, 1H), 4.2 (t, 2H), 3.78 (s, 3H), 3.6 (m, 1H), 3.5 (d, 1H), 3.32 (t, 1H), 3.28 (t, 1H), 3.11 (t, 1H), 3.1-2.9 (m, 4H), 3.02 (br s, 6H), 2.98 (m, 1H), 2.48 (s, 3H), 2.2-1.5 (m, 5H), 1.38 (d, 3H), 0.98 (d, 6H).

### Step 4:3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl-dimethyl-[[2-[2-[(2S,3R,4R,5S,6S)-6-carboxy-3,4,5-trihydroxy-tetrahydropyran-2-yl]ethyl]-4-[[(2S)-2-[[(2S)-2-[3-[2-(2,5-dioxopyrrol-1-yl)ethoxy]propanoylamino]-3-methyl-butanoyl]amino]propanoyl]amino]phenyl]methyl]ammonium; 2,2,2-trifluoroacetate

To a solution of the product from Step 3 (60 mg, 44.8 μmol) in solution in DMF (2.25 mL) were successively added (2,5-dioxopyrrolidin-1-yl) 3-[2-(2,5-dioxopyrrol-1-yl)ethoxy]propanoate (20.9 mg, 67.2 μmol) and DIPEA (23.4 μL, 134 μmol). The solution was stirred at room temperature for 3 h. The crude product was purified using C18 reverse phase prep-HPLC by direct deposit of the reaction mixture on the Xbridge® column and using the TFA method to give the desired product (28.5 mg). IR: (v cm⁻¹) 3600-3100, 2800-2200, 2234, 1705+1687+1614, 1537. ¹H NMR (400 MHz, dmso-d₆) δ ppm 12.5 (m, 2H), 10.5/8.20/7.90 (s+2d, 3H), 7.80 (d, 1H), 7.68 (2s, 2H), 7.60-7.40 (m, 4H), 7.40 (m, 2H), 7.20 (2t, 2H), 7.00 (s, 2H), 5.20-5.00 (m, 3H), 4.62/4.53 (2d, 2H), 4.50 (s, 2H), 4.38 (t, 1H), 4.20 (t, 4H), 3.80 (s, 3H), 3.60-3.00 (m, 10H), 3.02 (2s, 6H), 2.81 (m, 2H), 2.45 (s, 3H), 2.42/2.30 (2t, 4H), 2.15 (m, 2H), 2.00 (m, 1H), 1.95 (m, 2H), 1.30 (d, 3H), 0.89/0.82 (2d, 6H). HRMS (ESI) [M−CF₃CO₂]⁺=1306.4715 (δ=0.6 ppm).

### Preparation of L106C-P7: 2-[[6-(1,3-benzothiazol-2-ylamino)-5-methyl-pyridazin-3-yl]-methyl-amino]-5-[3-[4-[3-[[2-[2-[(2S,3R,4R,5S,6S)-6-carboxy-3,4,5-trihydroxy-tetrahydropyran-2-yl]ethyl]-4-[[(2S)-2-[[(2S)-2-[3-[2-(2,5-dioxopyrrol-1-yl)ethoxy]propanoylamino]-3-methyl-butanoyl]amino]propanoyl]amino]phenyl]methoxycarbonyl-methyl-amino]prop-1-ynyl]-2-fluoro-phenoxy]propyl]thiazole-4-carboxylic acid

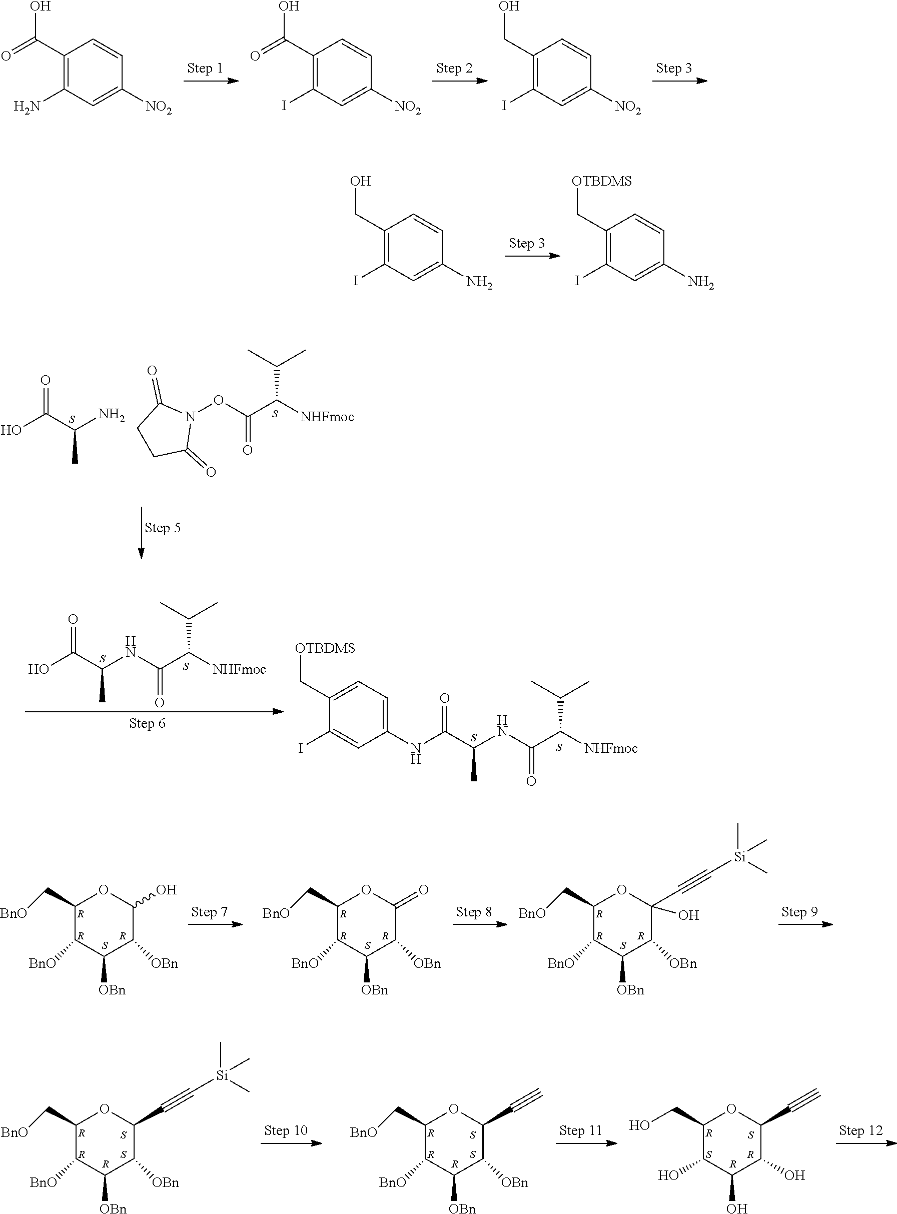

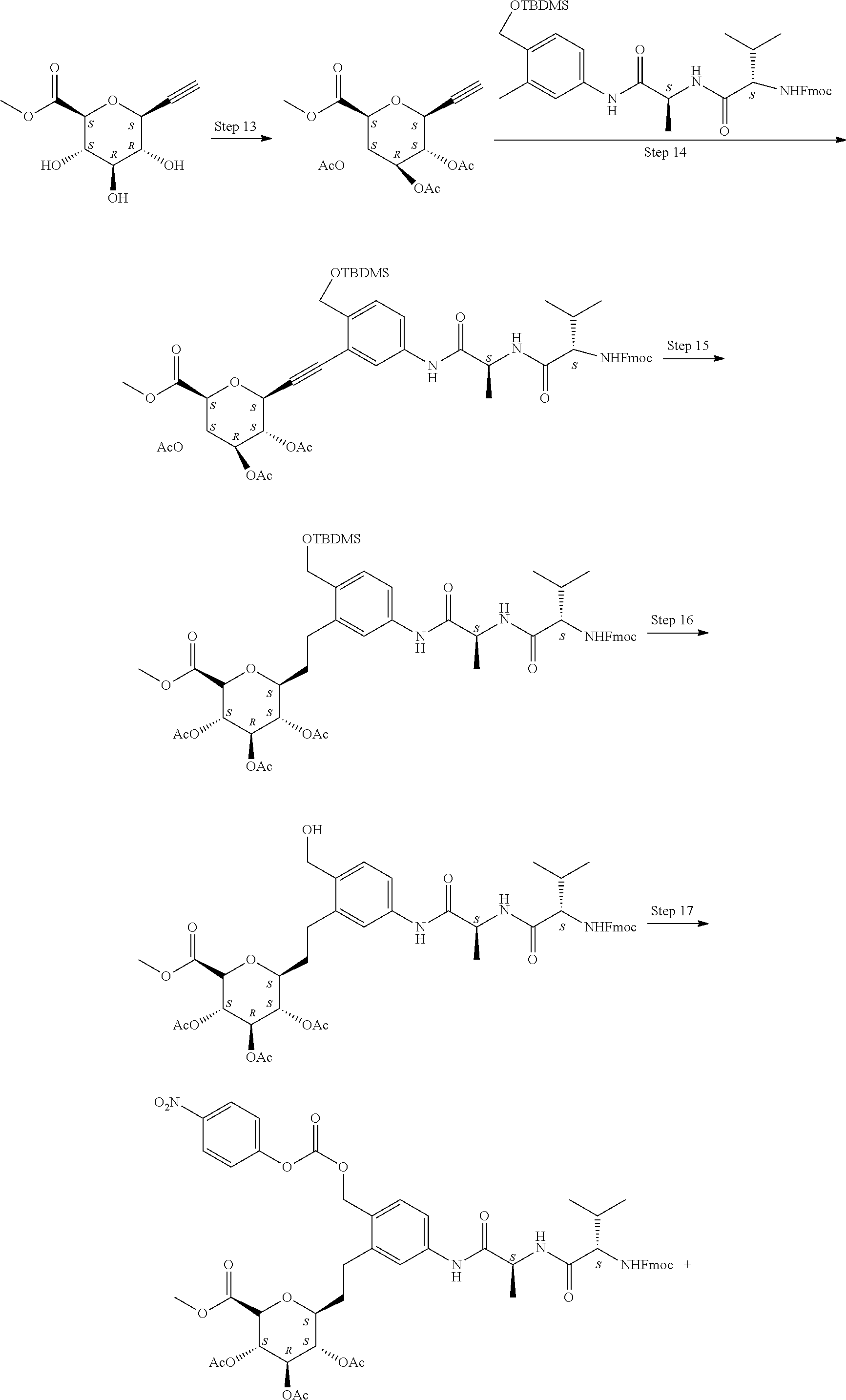

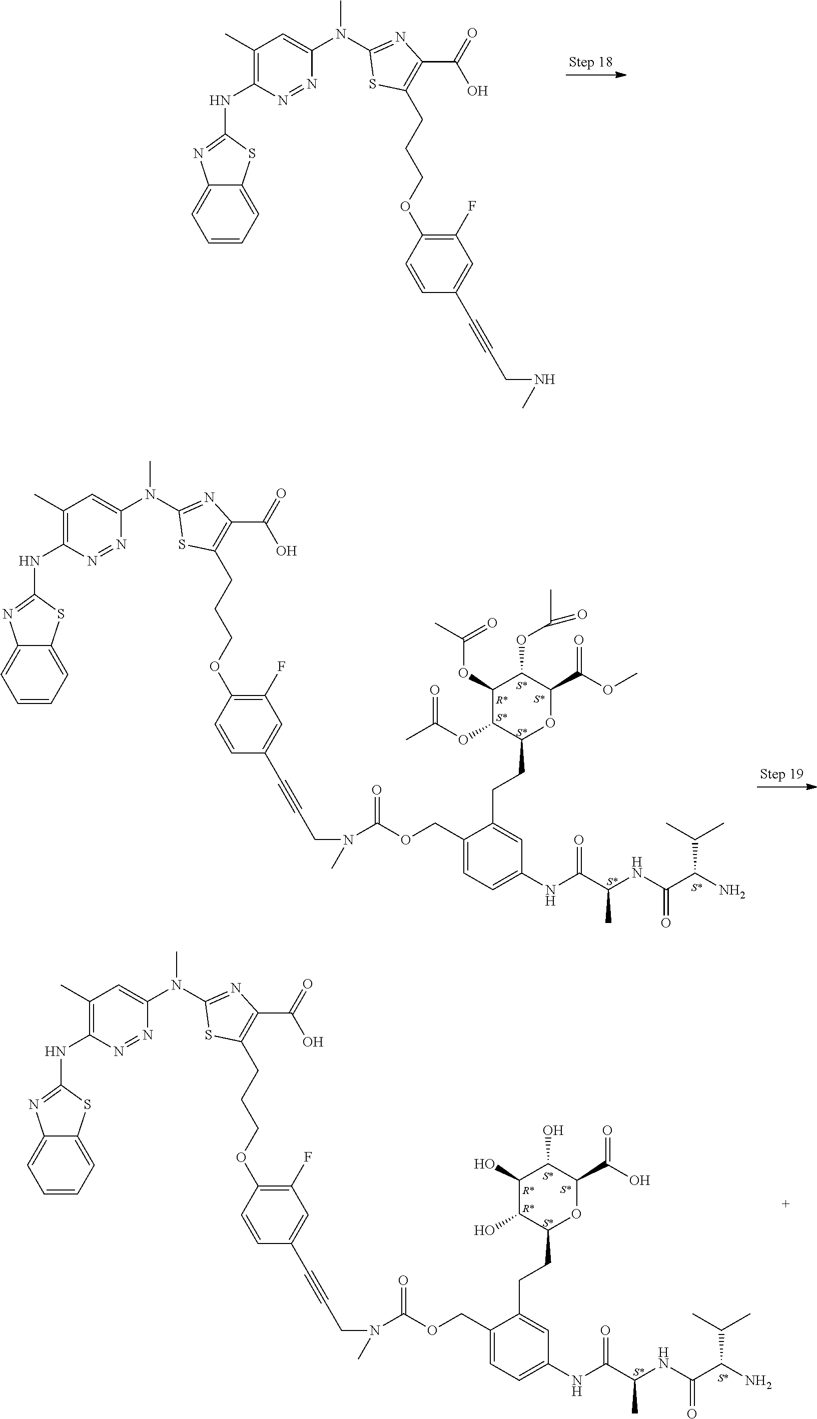 [SMILES: CNCC#Cc1ccc(OCCCc2sc(N(C)c3cc(C)c(Nc4nc5ccccc5s4)nn3)nc2C(=O)O)c(F)c1 ; COC(=O)[C@H]1O[C@@H](CCc2cc(NC(=O)[C@H](C)NC(=O)[C@@H](N)C(C)C)ccc2COC(=O)N(C)CC#Cc2ccc(OCCCc3sc(N(C)c4cc(C)c(Nc5nc6ccccc6s5)nn4)nc3C(=O)O)c(F)c2)[C@H](OC(C)=O)[C@@H](OC(C)=O)[C@@H]1OC(C)=O ; Cc1cc(N(C)c2nc(C(=O)O)c(CCCOc3ccc(C#CCN(C)C(=O)OCc4ccc(NC(=O)[C@H](C)NC(=O)[C@@H](N)C(C)C)cc4CC[C@@H]4O[C@H](C(=O)O)[C@@H](O)[C@H](O)[C@H]4O)cc3F)s2)nnc1Nc1nc2ccccc2s1 ; "+"]

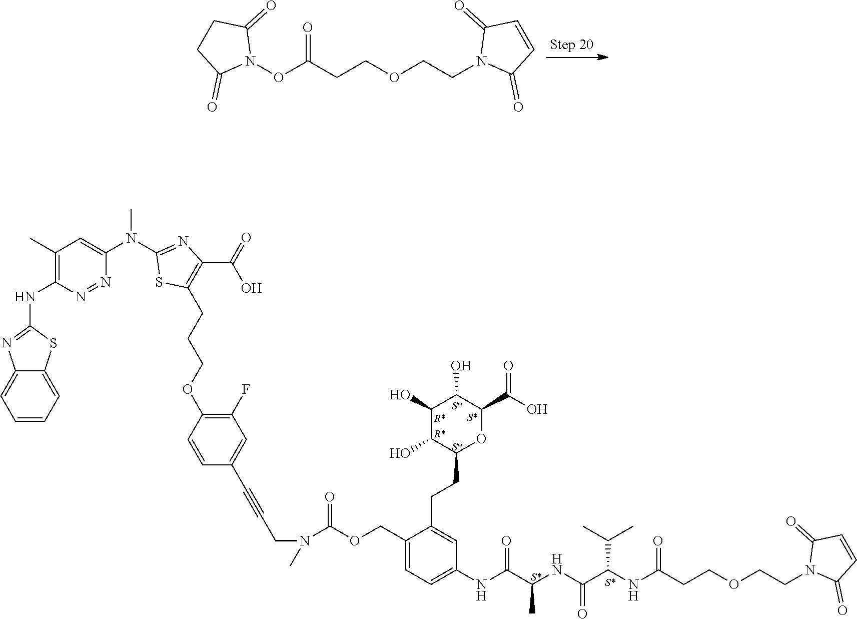 [SMILES: O=C(CCOCCN1C(=O)C=CC1=O)ON1C(=O)CCC1=O ; Cc1cc(N(C)c2nc(C(=O)O)c(CCCOc3ccc(C#CCN(C)C(=O)OCc4ccc(NC(=O)[C@H](C)NC(=O)[C@@H](NC(=O)CCOCCN5C(=O)C=CC5=O)C(C)C)cc4CC[C@@H]4O[C@H](C(=O)O)[C@@H](O)[C@H](O)[C@H]4O)cc3F)s2)nnc1Nc1nc2ccccc2s1]

### Step 1:2-iodo-4-nitro-benzoic acid

To a solution of 2-amino-4-nitro-benzoic acid (10.0 g, 54.90 mmol) in acetonitrile (280 mL) was added p-toluenesulfonic acid monohydrate (32.0 g, 168.2 mmol). The mixture was stirred at room temperature for 15 min, then a solution containing sodium nitrite (8.00 g, 115.9 mmol) and potassium iodide (24.0 g, 144.6 mmol) in water (140 mL) was added dropwise in 15 min. The reaction mixture was stirred for 19 h. After completion of the reaction, the mixture was quenched with sodium thiosulfate (13.02 g, 82.36 mmol) and acidified with an aqueous solution of hydrogen chloride 3 M (25 mL). The aqueous layer was extracted with ethyl acetate (2×250 mL) and the combined organic layers were washed with a 1 M aqueous solution of hydrogen chloride (100 mL), dried over sodium sulfate, filtered and concentrated to dryness. The resulting residue was taken up in dichloromethane (1 L) and washed with a 1 M aqueous solution of HCl (100 mL). The organic layer was dried over sodium sulfate, filtered, and concentrated to give the desired product (15.0 g). ¹H NMR (400 MHz, dmso-d₆): δ 13.8 (br s, 1H), 8.64 (s, 1H), 8.27 (d, 1H), 7.86 (d, 1H).

### Step 2: (2-iodo-4-nitro-phenyl)methanol

To a solution of the product from Step 1 (5.0 g, 17.06 mmol) in THF (70 mL) was added a 1 M solution of borane in THF (85 mL, 85 mmol). The reaction mixture was stirred at 65° C. for 4 h. The reaction mixture was cooled to room temperature and was quenched with the addition of methanol (200 mL). The mixture was stirred at room temperature for 30 min and concentrated to dryness. The crude product was purified by silica gel chromatography (gradient of ethyl acetate in cyclohexane) to give the desired product (3.38 g). ¹H NMR (400 MHz, dmso-d₆): δ 8.54 (d, 1H), 8.29 (dd, 1H), 7.70 (d, 1H), 5.82 (t, 1H), 4.47 (d, 2H).

### Step 3: (4-amino-2-iodo-phenyl)methanol

To a solution of the product from Step 2 (3.70 g, 13.26 mmol) in ethanol (100 mL) and water (25 mL) were successively added iron (3.70 g, 66.25 mmol) and ammonium chloride (800 mg, 14.96 mmol). The reaction mixture was stirred at 80° C. for 3 h. The reaction mixture was filtered over Celite®, washed with ethanol, and concentrated to dryness. The resulting residue was taken up in ethyl acetate (100 mL) and washed with a saturated solution of sodium hydrogen carbonate (100 mL), dried over sodium sulfate, filtered, and concentrated to dryness to give the desired product (2.48 g). ¹H NMR (400 MHz, dmso-d₆): δ 7.02-7.10 (m, 2H), 6.57 (d, 1H), 5.16 (s, 2H), 4.97 (t, 1H), 4.28 (d, 2H).

### Step 4: 4-[[tert-butyl(dimethyl)silyl]oxymethyl]-3-iodo-aniline

To a solution of the product from Step 3 (3.51 g, 13.37 mmol) in dichloromethane (150 mL) was added imidazole (0.95 g, 13.95 mmol). The mixture was cooled to 0° C. and a solution of tert-butyl-chloro-dimethyl-silane (2.40 mL, 13.85 mmol) in dichloromethane (150 mL) was added dropwise over 15 minutes. After stirring at room temperature for 16 h, the reaction mixture was quenched with methanol (20 mL) and concentrated. The crude product was purified by silica gel chromatography (gradient of ethyl acetate in cyclohexane) to give the desired product (3.64 g75%). ¹H NMR (400 MHz, dmso-d₆): δ 7.05 (s, 1H), 7.03 (d, 1H), 6.55 (d, 1H), 5.24 (s, 2H), 4.46 (s, 2H), 0.88 (s, 9H), 0.06 (s, 6H).

### Step 5: (2S)-2-[[(2S)-2-(9H-fluoren-9-ylmethoxycarbonylamino)-3-methyl-butanoyl]amino]propanoic acid

To a solution of (2S)-2-aminopropanoic acid (3.22 g, 36.09 mmol) in water (90 mL) were successively added sodium carbonate (7.29 g, 68.74 mmol) and a solution of (2,5-dioxopyrrolidin-1-yl)(2S)-2-(9H-fluoren-9-ylmethoxycarbonylamino)-3-methyl-butanoate (15.0 g, 34.37 mmol) in dimethoxyethane (90 mL). The reaction mixture was stirred at room temperature for 16 h. After acidification of the reaction until pH=1 with a 1 M aqueous solution of hydrogen chloride, the aqueous layer was extracted with ethyl acetate (3×500 mL). The combined organic layers were dried, concentrated, and triturated with diethyl ether (50 mL) to give the desired product (11.25 g). ¹H NMR (400 MHz, dmso-d₆) δ 12.48 (s, 1H), 8.21 (d, 1H), 7.89 (d, 2H), 7.72-7.79 (m, 2H), 7.28-7.46 (m, 5H), 4.15-4.32 (m, 4H), 3.90 (t, 1H), 1.90-2.02 (m, 1H), 1.28 (d, 3H), 0.86-0.90 (m, 6H).

### Step 6: 9H-fluoren-9-ylmethyl N-[(1S)-1-[[(1S)-2-[4-[[tert-butyl(dimethyl)silyl]oxymethyl]-3-iodo-anilino]-1-methyl-2-oxo-ethyl]carbamoyl]-2-methyl-propyl]carbamate

To a solution of the product from Step 5 (1.50 g, 3.65 mmol) in dichloromethane (18 mL) and methanol (18 mL) were successively added the product from Step 4 (1.33 g, 3.65 mmol) and ethyl 2-ethoxy-2H-quinoline-1-carboxylate (EEDQ)(1.36 g, 5.48 mmol). The suspension was stirred at room temperature for 16 h. After concentration, the crude product was purified by silica gel chromatography (gradient of ethyl acetate in cyclohexane) and then by C18 chromatography (gradient of methanol in water) to give the desired product (1.18 g). ¹H NMR (400 MHz, dmso-d₆): δ 10.05 (s, 1H). 8.16-8.24 (m, 2H), 7.88 (d, 2H), 7.71-7.77 (m, 2H), 7.55 (d, 1H), 7.37-7.48 (m, 3H), 7.27-7.37 (m, 3H), 4.56 (s, 2H), 4.38 (t, 1H), 4.18-4.33 (m, 3H), 3.91 (t, 1H), 2.08-2.20 (m, 1H), 1.30 (d, 3H), 0.83-0.95 (m, 15H), 0.06 (s, 6H).

### Step 7: (3R,4S,5R,6R)-3,4,5-tribenzyloxy-6-(benzyloxymethyl)tetrahydropyran-2-one

A suspension of (3R,4S,5R,6R)-3,4,5-tribenzyloxy-6-(benzyloxymethyl)tetrahydropyran-2-ol (30.0 g, 55.49 mmol) in DMSO (120 mL) was stirred at room temperature for 30 min and treated dropwise with acetic anhydride (90 mL) at room temperature over 15 min. The solution was stirred for 16 h, cooled to 0° C., and treated with a 1 M aqueous solution of hydrogen chloride (100 mL). The reaction mixture was stirred at room temperature for 20 min and the acetic acid was evaporated. The resulting residue was diluted with water (200 mL) and ethyl acetate (200 mL). The aqueous layer was extracted with ethyl acetate (2×200 mL) and the combined organic layers were washed with water (2×500 mL) and with a saturated solution of sodium hydrogen carbonate (2×500 mL), dried over sodium sulfate, filtered, concentrated, and purified by silica gel chromatography (gradient of ethyl acetate in cyclohexane) to give the desired product (25.05 g). ¹H NMR (400 MHz, dmso-d₆): δ 7.19-7.39 (m, 20H), 4.85 (d, 1H), 4.57-4.72 (m, 5H), 4.46-4.56 (m, 3H), 4.36 (d, 1H), 3.98-4.05 (m, 1H), 3.84-3.92 (m, 1H), 3.65-3.76 (m, 2H).

### Step 8: (3R,4S,5R,6R)-3,4,5-tribenzyloxy-6-(benzyloxymethyl)-2-(2-trimethylsilylethynyl)tetrahydropyran-2-ol

To a solution of trimethylsilylacetylene (24 mL, 168.6 mmol) in THF (325 mL) was added a 2.5 M solution of butyllithium in hexane (59.41 mL, 148.5 mmol) at −78° C. in 20 min. The solution was stirred at −78° C. for 45 min and at 0° C. for 45 min. The reaction mixture was cooled to −78° C. and a solution of the product from Step 7 (25.0 g, 46.41 mmol) in THF (325 mL) was added dropwise over 45 min. The reaction mixture was stirred at this temperature for 4 h and quenched with water (200 mL). The aqueous layer was extracted with ethyl acetate (2×200 mL). The combined organic layers were dried over sodium sulfate, filtered and concentrated to dryness to give the desired product (29.56 g) as a mixture of two diastereoisomers in a ratio 4/6. ¹H NMR (400 MHz, dmso-d₆): δ 7.13-7.43 (m, 20H), 4.87-4.99 (m, 1H), 4.65-4.83 (m, 4H), 3.43-3.57 (m, 3H), 3.70-3.85 (m, 2H), 3.55-3.68 (m, 3H), 3.43-3.53 (m, 2H), 0.11-0.22 (m, 9H).

### Step 9: trimethyl-[2-[(2S,3S,4R,5R,6R)-3,4,5-tribenzyloxy-6-(benzyloxymethyl)tetrahydropyran-2-yl]ethynyl]silane

To a solution of the product from Step 8 (29.56 g, 46.42 mmol) in acetonitrile (83 mL) and dichloromethane (193 mL) was added a solution of triethylsilane (44.98 mL, 278.5 mmol) in a mixture of acetonitrile/dichloromethane (37 mL/18 mL) in 20 min and a solution of boron trifluoride diethyl etherate (23.53 mL, 185.7 mmol) in acetonitrile (37 mL) in 30 min at −15° C. The solution was stirred for 5 h at the same temperature and diluted with water (500 mL). The aqueous layer was extracted with ethyl acetate (2×500 mL). The combined organic layers were dried over sodium sulfate, filtered and concentrated to dryness to give the desired product (28.82 g). ¹H NMR (400 MHz, dmso-d₆): δ 7.10-7.44 (m, 20H), 4.93 (d, 1H), 4.67-4.86 (m, 4H), 4.43-4.57 (m, 3H), 4.16-4.28 (m, 1H), 3.42-3.68 (m, 6H), 0.15 (s, 9H).

### Step 10: (2R,3R,4R,5S,6S)-3,4,5-tribenzyloxy-2-(benzyloxymethyl)-6-ethynyl-tetrahydropyran

To a solution of the product from Step 9 (28.80 g, 46.39 mmol) in methanol (1.12 L) and dichloromethane (240 mL) was added an 1 M aqueous solution of sodium hydroxide (80 mL). The solution was stirred at room temperature for 1 h, acidified until pH=1 with a 1 M aqueous solution of hydrogen chloride and diluted with water (500 mL). The methanol was evaporated and the aqueous layer was extracted with ethyl acetate (2×1 L). The combined organic layers were dried over sodium sulfate, filtered, concentrated and purified by silica gel chromatography (gradient of ethyl acetate in cyclohexane) to give the desired product (20.00 g). ¹H NMR (400 MHz, dmso-d₆): δ 3.42-3.67 (m, 7H), 4.17 (d, 1H), 4.44-4.56 (m, 3H), 4.67-4.86 (m, 4H), 4.90 (d, 1H), 7.15-7.40 (m, 20H).

### Step 11: (2S,3R,4R,5S,6R)-2-ethynyl-6-(hydroxymethyl)tetrahydropyran-3,4,5-triol

To a solution of the product from Step 10 (20.00 g, 36.45 mmol) in ethanethiol (400 mL) was added boron trifluoride diethyl etherate (147.8 mL, 1166 mmol) dropwise at room temperature over 5 min. The solution was stirred at room temperature for 16 h, cooled to 0° C., equipped with a gas trap containing an aqueous saturated solution of sodium hypochlorite, and treated dropwise with a saturated aqueous solution of sodium hydrogen carbonate (500 mL) at 0° C. in 1 h. After concentration to dryness, the crude product was purified by silica gel chromatography (gradient of methanol in dichloromethane) to give the desired product (4.05 g). ¹H NMR (400 MHz, dmso-d₆): δ 5.28 (d, 1H), 4.99 (d, 1H), 4.91 (d, 1H), 4.52 (t, 1H), 3.77 (d, 1H), 3.60-3.69 (m, 1H), 3.35-3.43 (m, 1H), 3.32 (s, 1H), 2.97-3.13 (m, 4H).

### Step 12: methyl (2S,3S,4R,5R,6S)-6-ethynyl-3,4,5-trihydroxy-tetrahydropyran-2-carboxylate

To a solution of the product from Step 11 (4.05 g, 21.52 mmol) in a saturated aqueous solution of sodium hydrogen carbonate (81 mL) and THF (81 mL) was added (2,2,6,6-tetramethylpipéridin-1-yl)oxyl (TEMPO)(168 mg, 1.08 mmol). The suspension was cooled to 0° C. and 1,3-dibromo-5,5-dimethyl-imidazolidine-2,4-dione (12.31 g, 43.04 mmol) was added portionwise in 30 min. The reaction mixture was stirred at 0° C. for 4 h and quenched with the addition of methanol (40 mL). After 30 min stirring at this temperature, a saturated aqueous solution of potassium carbonate (10 mL) and dichloromethane (100 mL) were added. After the organic layer was extracted with water (2×200 mL), the combined aqueous layers were acidified until pH=1 with a 3M aqueous solution of hydrogen chloride and concentrated to dryness. The residue was taken up in methanol (100 mL) and in a 3M aqueous solution of hydrogen chloride (20 mL). The mixture was concentrated and co-evaporated several times with methanol (4×100 mL). The crude product was purified by silica gel chromatography (gradient of methanol in dichloromethane Cerium developer) to give the desired product (3.00 g). ¹H NMR (400 MHz, dmso-d₆): δ 5.46 (d, 1H), 5.32 (d, 1H), 5.18 (d, 1H), 3.93-4.00 (m, 1H), 3.75 (dd, 1H), 3.65 (s, 3H), 3.40-3.44 (m, 1H), 3.31 (s, 1H), 3.09-3.19 (m, 2H).

### Step 13: methyl (2S,3S,4R,5S,6S)-3,4,5-triacetoxy-6-ethynyl-tetrahydropyran-2-carboxylate

To a solution of the product from Step 12 (3.00 g, 13.88 mmol) in DMF (37.5 mL) and pyridine (12.5 mL) was added N,N-dimethylpyridin-4-amine (DMAP)(84.8 mg, 0.693 mmol). The reaction mixture was cooled to 0° C. and treated with acetic anhydride (20.0 mL, 213 mmol) dropwise over 5 min. The solution was stirred at room temperature for 3 h and diluted with a 1 M aqueous solution of hydrogen chloride (200 mL). The aqueous layer was extracted with ethyl acetate (2×200 mL). The combined organic layers were washed with a 1M aqueous solution of hydrogen chloride (2×200 mL) and a saturated aqueous solution of potassium carbonate (200 mL), dried over sodium sulfate, filtered, concentrated and purified by silica gel chromatography (gradient of ethyl acetate in cyclohexane cerium developer) to give the desired product (4.60 g). ¹H NMR (400 MHz, dmso-d₆): δ 5.33 (t, 1H), 4.93-5.01 (m, 2H), 4.70 (d, 1H), 4.44 (d, 1H), 3.67 (s, 1H), 3.64 (s, 3H), 2.02 (s, 3H), 1.94-2.01 (m, 6H).

### Step 14: methyl (2S,3S,4R,5S,6S)-3,4,5-triacetoxy-6-[2-[2-[[tert-butyl(dimethyl)silyl]oxymethyl]-5-[[(2S)-2-[[(2S)-2-(9H-fluoren-9-ylmethoxycarbonylamino)-3-methyl-butanoyl]amino]propanoyl]amino]phenyl]ethynyl]tetrahydropyran-2-carboxylate

To a solution of the product from Step 13 (496 mg, 1.45 mmol) in DMF (7.3 mL) were successively added the product from Step 6 (730 mg, 0.966 mmol), DIPEA (738 μL, 4.47 mmol), copper iodide (18.4 mg, 96.6 mmol), and dichloro-bis-(triphenylphosphine)palladium(II) (67.8 mg, 96.6 mmol). The solution was flushed with argon and stirred at room temperature for 16 h. After dilution with water (100 mL), the aqueous layer was extracted with ethyl acetate (2×100 mL). The combined organic layers were washed with water (2×200 mL) and a saturated aqueous solution of ammonium chloride (2×200 mL), dried over sodium sulfate, filtered, concentrated, and purified by silica gel chromatography (gradient of ethyl acetate in cyclohexane) to give the desired product (782 mg). ¹H NMR (400 MHz, dmso-d₆): δ 10.09 (s, 1H). 8.20 (d, 1H), 7.89 (d, 2H), 7.70-7.78 (m, 3H), 7.55 (d, 1H), 7.32-7.46 (m, 4H), 7.27-7.32 (m, 2H), 5.41 (t, 1H), 4.96-5.14 (m, 3H), 4.67 (s, 2H), 4.51 (d, 1H), 4.36-4.44 (m, 1H), 4.16-4.32 (m, 3H), 3.88-3.95 (m, 1H), 3.64 (s, 3H), 1.94-2.07 (m, 10H), 1.30 (d, 3H), 0.84-0.93 (m, 15H), 0.08 (s, 6H).

### Step 15: methyl (3S,4R,5S,6S)-3,4,5-triacetoxy-6-[2-[2-[[tert-butyl(dimethyl)silyl]oxymethyl]-5-[[(2S)-2-[[(2S)-2-(9H-fluoren-9-ylmethoxycarbonylamino)-3-methyl-butanoyl]amino]propanoyl]amino]phenyl]ethyl]tetrahydropyran-2-carboxylate

A solution of the product from Step 14 (750 mg, 0.773 mmol) in THF (15 mL) was flushed with argon, treated with dry Platinum 5% on carbon (75 mg, 50% w/w), flushed successively with argon and with H₂, and stirred under H₂ atmosphere (1 atm) at room temperature for 16 h. The reaction mixture was filtered through a Celite® pad, washed with THF, and concentrated to dryness. The complete sequence (including addition of dry platinum 5% on carbon (75 mg, 50% w/w), stirring under H₂ atmosphere (1 atm) at room temperature for 16 h, and filtration through a Celite® pad) was performed 4 more times. The crude product was purified by silica gel chromatography (gradient of ethyl acetate in cyclohexane) to give the desired product (470 mg). ¹H NMR (400 MHz, dmso-d₆): δ 9.90 (s, 1H), 8.16 (d, 1H), 7.89 (d, 2H), 7.70-7.78 (m, 2H), 7.37-7.49 (m, 4H), 7.27-7.32 (m, 3H), 7.23 (d, 1H), 5.29 (t, 1H), 4.95 (t, 1H), 4.78 (t, 1H), 4.60 (s, 2H), 4.34-4.44 (m, 2H), 4.16-4.32 (m, 3H), 3.88-3.95 (m, 1H), 3.72-3.79 (m, 1H), 3.64 (s, 3H), 2.69-2.78 (m, 1H), 2.50-2.60 (m, 1H), 1.92-2.03 (m, 10H), 1.55-1.75 (m, 2H), 1.30 (d, 3H), 0.84-0.93 (m, 15H), 0.05 (s, 6H).

### Step 16: methyl (3S,4R,5S,6S)-3,4,5-triacetoxy-6-[2-[5-[[(2S)-2-[[(2S)-2-(9H-fluoren-9-ylmethoxycarbonylamino)-3-methyl-butanoyl]amino]propanoyl]amino]-2-(hydroxymethyl)phenyl]ethyl]tetrahydropyran-2-carboxylate

To a solution of the product from Step 15 (470 mg, 0.483 mmol) in THF (540 μL) and water (540 μL) was added acetic acid (1.6 mL, 28.28 mmol). The solution was stirred at room temperature for 16 h and diluted with water (100 mL). The aqueous layer was extracted with ethyl acetate (2×100 mL). The combined organic layers were washed with water (2×200 mL) and a saturated aqueous solution of sodium hydrogen carbonate (200 mL), dried over sodium sulfate, filtered, concentrated, and purified by silica gel chromatography (gradient of ethyl acetate in cyclohexane) to give the desired product (354 mg). ¹H NMR (400 MHz, dmso-d₆): δ 9.87 (s, 1H), 8.16 (d, 1H), 7.89 (d, 2H), 7.70-7.78 (m, 2H), 7.37-7.50 (m, 4H), 7.27-7.37 (m, 3H), 7.25 (d, 1H), 5.29 (t, 1H), 4.91-4.98 (m, 2H), 4.78 (t, 1H), 4.34-4.44 (m, 4H), 4.16-4.32 (m, 3H), 3.88-3.95 (m, 1H), 3.72-3.79 (m, 1H), 3.64 (s, 3H), 2.64-2.73 (m, 1H), 2.50-2.60 (m, 1H), 1.92-2.03 (m, 10H), 1.69-1.79 (m, 1H), 1.52-1.65 (m, 1H), 1.30 (d, 3H), 0.84-0.93 (m, 6H).

### Step 17: methyl (3S,4R,5S,6S)-3,4,5-triacetoxy-6-[2-[5-[[(2S)-2-[[(2S)-2-(9H-fluoren-9-ylmethoxycarbonylamino)-3-methyl-butanoyl]amino]propanoyl]amino]-2-[(4-nitrophenoxy)carbonyloxymethyl]phenyl]ethyl]tetrahydropyran-2-carboxylate

To a solution of the product from Step 16 (310 mg, 0.361 mmol) in THF (7.75 mL) were successively added pyridine (146 μL, 1.80 mmol) and 4-nitrophenyl chlorocarbonate (182 mg, 0.901 mmol). The suspension was stirred at room temperature for 16 h, concentrated, and purified by silica gel chromatography (gradient of ethyl acetate in dichloromethane) to give the desired product (257 mg). ¹H NMR (400 MHz, dmso-d₆): δ 10.04 (s, 1H), 8.31 (d, 2H), 8.20 (d, 1H), 7.89 (d, 2H), 7.66-7.78 (m, 2H), 7.56 (d, 2H), 7.28-7.52 (m, 8H), 5.31 (t, 1H), 5.25 (s, 2H), 4.96 (t, 1H), 4.79 (t, 1H), 4.40 (d, 2H), 4.16-4.32 (m, 3H), 3.88-3.95 (m, 1H), 3.74-3.83 (m, 1H), 3.61 (s, 3H), 2.74-2.84 (m, 1H), 2.60-2.71 (m, 1H), 1.90-2.03 (m, 10H), 1.72-1.83 (m, 1H), 1.58-1.71 (m, 1H), 1.30 (d, 3H), 0.82-0.94 (m, 6H). LC-MS: MS (ESI) m/z [M+Na]⁺=1047.6.

### Step 18: 2-[[6-(1,3-benzothiazol-2-ylamino)-5-methyl-pyridazin-3-yl]-methyl-amino]-5-[3-[2-fluoro-4-[3-[methyl-[[4-[[(2S)-2-[[(2S)-2-amino-3-methyl-butanoyl]amino]propanoyl]amino]-2-[2-[(2S,3S,4R,5S,6S)-3,4,5-triacetoxy-6-methoxycarbonyl-tetrahydropyran-2-yl]ethyl]phenyl]methoxycarbonyl]amino]prop-1-ynyl]phenoxy]propyl]thiazole-4-carboxylic acid

To a solution of the product from Step 17 (130 mg, 127 μmol) in DMF (1.5 mL) were successively added a solution of 2-[[6-(1,3-benzothiazol-2-ylamino)-5-methyl-pyridazin-3-yl]-methyl-amino]-5-[3-[2-fluoro-4-[3-(methylamino)prop-1-ynyl]phenoxy]propyl]thiazole-4-carboxylic acid (P7)(101 mg, 168 μmol) in DMF (1.5 mL) and DIPEA (83 μL, 502 μmol). The reaction mixture was stirred 4 h at room temperature. The crude product was purified using C18 reverse phase prep-HPLC by direct deposit of the reaction mixture on the Xbridge® column and using the NH₄HCO₃ method to give the desired product (80 mg).

### Step 19: 2-[[6-(1,3-benzothiazol-2-ylamino)-5-methyl-pyridazin-3-yl]-methyl-amino]-5-[3-[2-fluoro-4-[3-[methyl-[[2-[2-[(2S,3R,4R,5S,6S)-6-carboxy-3,4,5-trihydroxy-tetrahydropyran-2-yl]ethyl]-4-[[(2S)-2-[[(2S)-2-amino-3-methyl-butanoyl]amino]propanoyl]amino]phenyl]methoxycarbonyl]amino]prop-1-ynyl]phenoxy]propyl]thiazole-4-carboxylic acid

To the solution of the product from Step 18 (80 mg, 62.4 μmol) in DMF (2.0 mL) was added and lithium hydroxide monohydrate (31.5 mg, 750 μmol) in water (500 μL). The reaction mixture was stirred at room temperature for 2 h. The crude product was purified using C18 reverse phase prep-HPLC by direct deposit of the reaction mixture on the Xbridge® column and using the NH₄HCO₃ method to give the desired product (25 mg).

### Step 20: 2-[[6-(1,3-benzothiazol-2-ylamino)-5-methyl-pyridazin-3-yl]-methyl-amino]-5-[3-[2-fluoro-4-[3-[methyl-[[2-[2-[(2S,3R,4R,5S,6S)-6-carboxy-3,4,5-trihydroxy-tetrahydropyran-2-yl]ethyl]-4-[[(2S)-2-[[(2S)-2-[3-[2-(2,5-dioxopyrrol-1-yl)ethoxy]propanoylamino]-3-methyl-butanoyl]amino]propanoyl]amino]phenyl]methoxycarbonyl]amino]prop-1-ynyl]phenoxy]propyl]thiazole-4-carboxylic acid

To a solution of the product from Step 19 (25 mg, 21.9 μmol) in DMF (1 mL) were successively added (2,5-dioxopyrrolidin-1-yl) 3-[2-(2,5-dioxopyrrol-1-yl)ethoxy]propanoate (11.1 mg, 32.9 μmol) and DIPEA (5.4 μL, 32.9 μmol). The solution was stirred at room temperature for 1 h. The crude product was purified using C18 reverse phase prep-HPLC by direct deposit of the reaction mixture on the Xbridge® column and using the TFA method to give the desired product (5 mg). HRMS (ESI) [M+H]⁺ found=1336.4453 (δ=0.3 ppm).

### Preparation of L108A-P2: 3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl-[[4-[(2S,3R,4S,5S,6S)-6-carboxy-3,4,5-trihydroxy-tetrahydropyran-2-yl]oxy-3-[3-[3-[2-(2,5-dioxopyrrol-1-yl)ethoxy]propanoylamino]propanoylamino]phenyl]methyl]-dimethyl-ammonium;2,2,2-trifluoroacetate

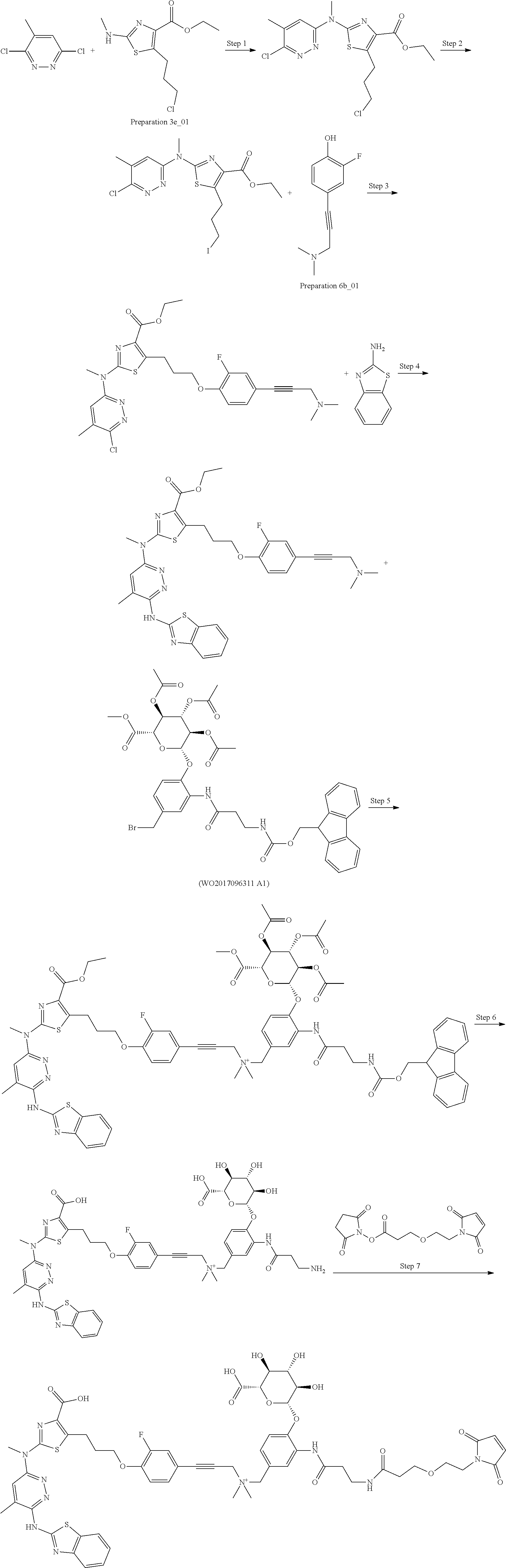

### Step 1: ethyl 2-[(6-chloro-5-methyl-pyridazin-3-yl)-methyl-amino]-5-(3-chloropropyl)thiazole-4-carboxylate

To a solution of ethyl 5-(3-chloropropyl)-2-(methylamino)thiazole-4-carboxylate (from Preparation 3e_01, 15.44 g, 58.5 mmol) in THF (600 mL) cooled to 0° C. was added at 0° C. NaH (60% in oil)(2.8 g, 70.6 mmol) in portion over a 0.5 h time period. The suspension was stirred at 0° C. for 0.5 h. To this suspension was then added dropwise at 0° C. a solution of 3,6-dichloro-4-methyl-pyridazine (23.0 g, 141 mmol) in solution in THF (200 mL). The reaction mixture was stirred at room temperature for 15 h, cooled to 0° C. and then water (25 mL) was slowly added.

### The aqueous layer was extracted 3 times with AcOEt and the organic layer dried over MgSO₄. The crude product was purified by silica gel chromatography (gradient of AcOEt in petroleum ether) to give the desired product (7.0 g, 18.0 μmol). IR: (v cm⁻¹) 3450, 1698, 1203. ¹H NMR (400 MHz, dmso-d₆) δ ppm 7.81 (s, 1H), 4.3 (quad, 2H), 3.78 (s, 3H), 3.31 (t, 2H), 3.2 (m, 2H), 2.4 (s, 3H), 2.12 (quint, 2H), 1.31 (t, 3H)

### Step 2: ethyl 2-[(6-chloro-5-methyl-pyridazin-3-yl)-methyl-amino]-5-(3-iodopropyl)thiazole-4-carboxylate

To a solution of the product from Step 1 (7.0 g, 18.0 mmol) in acetone (120 mL) was added sodium iodide (27 g, 178 mmol) and the suspension was heated at reflux (60° C.) for 15 h. After the reaction mixture was cooled to room temperature, the precipitate was filtered, washed with acetone and the filtrate was evaporated to dryness. The resulting yellow solid was triturated with ether, filtered and dried over phosphorous pentoxide (P₂O₅) at 35° C. for 48 h to give the desired product (7.6 g, 15.8 mmol) as a brown solid. IR: (v cm⁻¹) 1703, 1591. ¹H NMR (400 MHz, dmso-d₆) δ ppm 7.82 (df, 1H), 7.28 (dd, 1H), 7.2 (dd, 1H), 7.13 (t, 1H), 4.26 (q, 2H), 4.12 (t, 2H), 3.77 (s, 3H), 3.41 (s, 2H), 3.26 (t, 2H), 2.42 (s, 3H), 2.22 (s, 6H), 2.11 (m, 2H), 1.29 (t, 3H).

### Step 3: ethyl 2-[(6-chloro-5-methyl-pyridazin-3-yl)-methyl-amino]-5-[3-[4-[3-(dimethylamino)prop-1-ynyl]-2-fluoro-phenoxy]propyl]thiazole-4-carboxylate

To a solution of product from Step 2 (3.5 g, 7.28 mmol) in THF (400 mL) were successively added a solution of 4-[3-(dimethylamino)prop-1-ynyl]-2-fluoro-phenol (from Preparation 6b_01, 1.74 g, 8.74 mmol) in THF (100 mL) and cesium carbonate (Cs₂CO₃)(4.73 g, 8.74 mmol). The reaction mixture was heated at reflux (70° C.) for 15 h. The reaction mixture was cooled to room temperature, poured into water (100 mL) and extracted 3 times with AcOEt. The organic layer was washed with brine, dried over MgSO₄ and evaporate to dryness. The crude product was purified by silica gel chromatography (gradient of methanol in DCM) to afford the desired product (2.40 g, 4.39 mmol). IR: (v cm⁻¹) 1698, 1H NMR (400/500 MHz, dmso-d₆) δ ppm 7.8 (s, 1H), 4.3 (quad, 2H), 3.8 (s, 3H), 3.7 (t, 2H), 3.2 (m, 2H), 2.4 (s, 3H), 2.1 (quint, 2 H), 1.3 (t, 3H).

### Step 4: ethyl 2-[[6-(1,3-benzothiazol-2-ylamino)-5-methyl-pyridazin-3-yl]-methyl-amino]-5-[3-[4-[3-(dimethylamino)prop-1-ynyl]-2-fluoro-phenoxy]propyl]thiazole-4-carboxylate

To a solution saturated with argon of the product from Step 3 (961 mg, 1.76 mmol) and 1,3-benzothiazol-2-amine (317 mg, 2.11 mmol) in NMP (10 mL) were successively added 4,5-Bis(diphenylphosphino)-9,9-dimethylxanthene (Xantphos)(509 mg, 0.88 mmol) and tris(dibenzylideneacetone)dipalladium(0) (Pd₂(dba)₃)(12.9 mg, 0.044 mmol). The reaction mixture was again saturated with argon for 15 min, DIEPA (1 mL, 5.28 mmol) was added and the reaction mixture was stirred at 150° C. for 15 h. The reaction mixture was cooled to room temperature, water was added, and the aqueous phase was extracted several times with DCM. The organic phases were collected, washed with brine, dried over MgSO₄ and evaporated to dryness. The crude product was purified by silica gel chromatography (gradient of methanol in DCM) the desired compound (540 mg, 0.818 mmol). IR: (v cm⁻¹) 3700-2300, 1706. ¹H NMR (400 MHz, dmso-d₆) δ ppm 11.55 (m, 1H), 7.91 (d, 1H), 7.68 (s, 1H), 7.53 (d, 1H), 7.39 (m, 1 H), 7.3 (dd, 1H), 7.26-7.13 (m, 3H), 4.26 (q, 2H), 4.15 (t, 2H), 3.77 (s, 3H), 3.4 (s, 2H), 3.27 (m, 2H), 2.46 (s, 3H), 2.21 (s, 6H).

### Step 5:3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl-[[3-[3-(9H-fluoren-9-ylmethoxycarbonylamino)propanoylamino]-4-[(2S,3R,4S,5S,6S)-3,4,5-triacetoxy-6-methoxycarbonyl-tetrahydropyran-2-yl]oxy-phenyl]methyl]-dimethyl-ammonium

To a solution of the product from Step 4 (75 mg, 0.119 mmol) in DMF (2 mL) was added DIPEA (40 μL, 0.237 mmol) and methyl (2S,3S,4S,5R,6S)-3,4,5-triacetoxy-6-[4-(bromomethyl)-2-[3-(9H-fluoren-9-ylmethoxycarbonylamino)propanoylamino]phenoxy]tetrahydropyran-2-carboxylate (WO2017096311A1, 128 mg, 0.158 mmol) and the reaction was stirred at room temperature for 2 h. The crude product was purified using C18 reverse phase prep-HPLC by direct deposit of the reaction mixture on the Xbridge® column and using the TFA method to give the desired compound (88 mg, 51% yield). ¹H NMR (400 MHz, dmso-d₆) δ ppm 8.9/8.2/7.35 (2s+m, 3H), 7.9-7.2 (m, 11H), 7.88 (d, 2H), 7.68 (d, 2H), 7.4/7.3 (2t, 4H), 5.7 (d, 1H), 5.52 (t, 1H), 5.21 (t, 1H), 5.1 (t, 1H), 4.78 (d, 1H), 4.52/4.4 (2s, 4H), 4.3-4.15 (m, 7H), 3.78 (s, 3H), 3.62 (s, 3H), 3.3 (m, 4H), 3.08 (s, 6H), 2.55 (m, 2H), 2.48 (s, 3H), 2.15 (m, 2H), 2.01 (3s, 9 H), 1.3 (t, 3H). LCMS m/z=660.

### Step 6: [3-(3-aminopropanoylamino)-4-[(2S,3R,4S,5S,6S)-6-carboxy-3,4,5-trihydroxy-tetrahydropyran-2-yl]oxy-phenyl]methyl-[3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl]-dimethyl-ammonium

To a solution of the product of Step 5 (85 mg, 0.06 mmol) in MeOH (4 mL) was added LiOH dihydrate (64 mg, 1.53 mmol) and the reaction was stirred at room temperature for 5 h. The crude product was purified by Porapack® using NH₃/MeOH 7N as an eluent to give the desired compound (55 mg, 91% yield).

### Step 7:3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl-[[4-[(2S,3R,4S,5S,6S)-6-carboxy-3,4,5-trihydroxy-tetrahydropyran-2-yl]oxy-3-[3-[3-[2-(2,5-dioxopyrrol-1-yl)ethoxy]propanoylamino]propanoylamino]phenyl]methyl]-dimethyl-ammonium;2,2,2-trifluoroacetate

To a solution of product of Step 6 (50 mg, 0.05 mmol) in DMF (6 mL) were successively added DIPEA (30 μL, 0.179 mmol) and (2,5-dioxopyrrolidin-1-yl) 3-[2-(2,5-dioxopyrrol-1-yl)ethoxy]propanoate (28 mg, 0.09 mmol). The solution was stirred at room temperature for 1.5 h. The crude product was purified using C18 reverse phase prep-HPLC by direct deposit of the reaction mixture on the Xbridge® column and using the TFA method to give the desired product (15 mg, 20% yield). ¹H NMR (400 MHz, dmso-d₆) δ ppm 8.4 (br s, 1H), 7.9 (m, 1H), 7.7 (br s, 1 H), 7.6 (dd, 1H), 7.5 (dl, 1H), 7.45 (dl, 1H), 7.4 (td, 1H), 7.25 (m, 3H), 7.2 (t, 1H), 7 (s, 2H), 5 (d, 1H), 4.55/4.4 (2 br s, 4H), 4.2 (t, 2H), 4 (d, 1H), 3.8 (s, 3H), 3.55 (2t, 4H), 3.45 (m, 2H), 3.45/3.4 (2m, 3H), 3.35 (m, 2H), 3.3 (t, 2H), 3.1 (br s, 6H), 2.6 (t, 2H), 2.45 (s, 3H), 2.15 (t, 2 H), 2.15 (quint, 2H). ¹⁹F NMR (400 MHz, dmso-d₆) δ ppm −133.8. HRMS (ESI) [M−CF₃CO₂]⁺ found=1195.3690 (δ=2.5 ppm)

### Preparation of L107C-P7: 2-[[6-(1,3-benzothiazol-2-ylamino)-5-methyl-pyridazin-3-yl]-methyl-amino]-5-[3-[4-[3-[[4-[[(2S)-2-[[(2S)-2-[3-[2-[3-(2,5-dioxopyrrol-1-yl)propanoylamino]ethoxy]propanoylamino]-3-methyl-butanoyl]amino]propanoyl]amino]phenyl]methoxycarbonyl-methyl-amino]prop-1-ynyl]-2-fluoro-phenoxy]propyl]thiazole-4-carboxylic acid

Product was synthesized according to Method G by replacing 2-[2-[2-(2-azidoethoxy)ethoxy]ethoxy]acetic acid with 3-[2-[2-[2-[2-[2-[2-[2-[2-[2-[2-[2-(2-azidoethoxy)ethoxy]ethoxy]ethoxy]ethoxy]ethoxy]ethoxy]ethoxy]ethoxy]ethoxy]ethoxy]ethoxy]pr opanoic acid. ¹H NMR (400 MHz, dmso-d₆) δ ppm 12.55 (br s, 1H), 11.5-10.8 (diffus, 1H), 9.92 (s, 1H), 8.16 (d, 1H), 7.99 (t, 1H), 7.9 (diffus, 1H), 7.86 (d, 1H), 7.67 (br s, 1H), 7.64 (diffus, 1H), 7.58 (d, 2H), 7.38/7.2 (2m, 3H), 7.35 (m, 1H), 7.32 (d, 2H), 7.15 (t, 1H), 7 (s, 2H), 5.03 (s, 2H), 4.39 (quint, 1H), 4.28 (s, 2H), 4.2 (dd, 1H), 4.15 (t, 2H), 3.77 (s, 3H), 3.59 (t, 4H), 3.5 (m, 44H), 3.36 (t, 2H), 3.28 (t, 2H), 3.14 (quad, 2H), 2.9 (s, 3H), 2.49 (s, 3H), 2.45/2.33 (2t, 4H), 2.13 (quint, 2H), 1.96 (oct, 1 H), 1.3 (d, 3H), 0.87/0.83 (2d, 6H). HRMS (ESI) [M+H]⁺ found=1 687.7071 (δ=0).

### Preparation of L107A-P2: 3-[4-[3-[2-[[6-(1,3-benzothiazol-2-ylamino)-5-methyl-pyridazin-3-yl]-methyl-amino]-4-carboxy-thiazol-5-yl]propoxy]-3-fluoro-phenyl]prop-2-ynyl-[[4-[[(2S)-2-[[(2S)-2-[3-[2-[3-(2,5-dioxopyrrol-1-yl)propanoylamino]ethoxy]propanoylamino]-3-methyl-butanoyl]amino]propanoyl]amino]phenyl]methyl]-dimethyl-ammonium;2,2,2-trifluoroacetate

The desired product was obtained using Method A. (2S)-2-amino-N-[(1S)-2-[4-(hydroxymethyl)anilino]-1-methyl-2-oxo-ethyl]-3-methyl-butanamide and (2,5-dioxopyrrolidin-1-yl) 3-[2-[2-[2-[2-[2-[2-[2-[2-[2-[2-[2-[2-[3-(2,5-dioxopyrrol-1-yl)propanoylamino]ethoxy]ethoxy]ethoxy]ethoxy]ethoxy]ethoxy]ethoxy]ethoxy]ethoxy]ethoxy]ethoxy]ethoxy]propanoate was used in Step 1, and P2 was used as the appropriate payload in Step 3. ¹H NMR (400 MHz, dmso-d₆) δ ppm 10.2 (s), 8.23 (d), 7.99 (t), 7.89 (large, 1H), 7.85 (d), 7.76 (d, 2H), 7.67 (s, 1H), 7.56 (d, 1H), 7.5 (d, 2H), 7.4 (t, 1H), 7.38 (m, 2H), 7.24 (t, 1H), 7.2 (t, 1H), 6.99 (s, 2H), 4.55 (s, 2H), 4.41 (s, 2H), 4.39 (m, 1H), 4.2 (m, 1H), 4.19 (m, 2H), 3.77 (s, 3H), 3.65-3.33 (m, 24H), 3.59 (m, 2H), 3.29 (t, 2H), 3.14 (quad, 2H), 3.05 (s, 6H), 2.46 (s, 3H), 2.39 (m, 2H), 2.33 (t, 2H), 2.15 (m, 2H), 1.96 (m, 1H), 1.32 (d, 3H), 0.89/0.84 (2d, 6H). ¹³C NMR (400 MHz, dmso-d₆) δ ppm 134.7, 134.2, 126, 122.9, 122.2, 119.8, 119.7, 119.4, 118.3, 115.5, 70.4/69.2/67.2, 69, 66.8, 58.1, 53.9, 49.9, 49.9/40.4, 39, 36.4, 35.4, 34.6, 34.6, 31.1, 31.1, 23.6, 20.1, 18.2, 18.1. HRMS (ESI) [M+H]⁺ found=1 657.7339 (δ=0.4).

### Preparation of L9C-P59: 2-[3-(1,3-benzothiazol-2-ylamino)-4-methyl-6,7-dihydro-5H-pyrido[2,3-c]pyridazin-8-yl]-5-[3-[4-[3-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-(3-hydroxypropyl)amino]prop-1-ynyl]-2-fluoro-phenoxy]propyl]thiazole-4-carboxylic acid

Using Method C and P59 as the appropriate payload, the desired product was obtained. HRMS (ESI) [M+H]⁺ found=1288.4656 (δ=−4.5 ppm).

### Preparation of L9C-P3: 2-[3-(1,3-benzothiazol-2-ylamino)-4-methyl-6,7-dihydro-5H-pyrido[2,3-c]pyridazin-8-yl]-5-[3-[4-[3-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-methyl-amino]prop-1-ynyl]-2-fluoro-phenoxy]propyl]thiazole-4-carboxylic acid

Using Method C and P3 as the appropriate payload, the desired product was obtained. HRMS (ESI) [M+H]⁺ found=1244.4473 (δ=1.7 ppm).

### Preparation of L9C-P60: 6-[3-(1,3-benzothiazol-2-ylamino)-4-methyl-6,7-dihydro-5H-pyrido[2,3-c]pyridazin-8-yl]-3-[1-[[(5SR,7RS)-3-[2-[[(3S)-3,4-dihydroxybutyl]-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl]amino]ethoxy]-1-adamantyl]methyl]-5-methyl-pyrazol-4-yl]pyridine-2-carboxylic acid

Using Method C and P60 as the appropriate payload, the desired product was obtained. HRMS (ESI) [M+H]⁺ found=1394.6300 (δ=−3.6 ppm).

### Preparation of L9A-P61: 6-[3-(1,3-benzothiazol-2-ylamino)-4-methyl-6,7-dihydro-5H-pyrido[2,3-c]pyridazin-8-yl]-3-[1-[[(5RS,7SR)-3-[2-[1-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]pyrrolidin-1-ium-1-yl]ethoxy]-1-adamantyl]methyl]-5-methyl-pyrazol-4-yl]pyridine-2-carboxylic acid; 2,2,2-trifluoroacetate

Using Method A and P61 as the appropriate payload, the desired product was obtained. HRMS (ESI) [M]⁺ found=1316.6347 (δ=−3.8 ppm).

### Preparation of L9A-P62: 2-[[(5RS,7SR)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]oxy]ethyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-(3-hydroxypropyl)-methyl-ammonium; 2,2,2-trifluoroacetate

Using Method B and P62 as the appropriate payload, the desired product was obtained. HRMS (ESI) [M]⁺ found=1362.6748 (δ=−5.0 ppm).

### Preparation of L9A-P63: 3-[1-[[(5SR,7RS)-3-[2-[1-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]pyrrolidin-1-ium-1-yl]ethoxy]-5,7-dimethyl-1-adamantyl]methyl]-5-methyl-pyrazol-4-yl]-6-[3-[(5-fluoro-1,3-benzothiazol-2-yl)amino]-4-methyl-6,7-dihydro-5H-pyrido[2,3-c]pyridazin-8-yl]pyridine-2-carboxylic acid; 2,2,2-trifluoroacetate

Using Method A and P63 as the appropriate payload, the desired product was obtained. HRMS (ESI) [M]⁺ found=1362.6585 (δ=−2.3 ppm).

### Preparation of L9A-P64: 3-[1-[[(5RS,7SR)-3-[2-[1-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]pyrrolidin-1-ium-1-yl]ethoxy]-5,7-dimethyl-1-adamantyl]methyl]-5-methyl-pyrazol-4-yl]-6-[4-methyl-3-[(6-methyl-1,3-benzothiazol-2-yl)amino]-6,7-dihydro-5H-pyrido[2,3-c]pyridazin-8-yl]pyridine-2-carboxylic acid; 2,2,2-trifluoroacetate

Using Method A and P64 as the appropriate payload, the desired product was obtained. HRMS (ESI) [M]+ found=1358.6809 (δ=−4.3 ppm).

### Preparation of L9A-P65: 3-[1-[[(5SR,7RS)-3-[2-[1-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]pyrrolidin-1-ium-1-yl]ethoxy]-5,7-dimethyl-1-adamantyl]methyl]-5-methyl-pyrazol-4-yl]-6-[3-[(6-fluoro-1,3-benzothiazol-2-yl)amino]-4-methyl-6,7-dihydro-5H-pyrido[2,3-c]pyridazin-8-yl]pyridine-2-carboxylic acid; 2,2,2-trifluoroacetate

Using Method A and P65 as the appropriate payload, the desired product was obtained. HRMS (ESI) [M]+ found=1362.6557 (δ=−4.3 ppm).

### Preparation of L9A-P66: 3-[(5RS,7SR)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]propyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-dimethyl-ammonium; 2,2,2-trifluoroacetate

Using Method A and P66 as the appropriate payload, the desired product was obtained. HRMS (ESI) [M]+ found=1316.6703 (δ=−4.4 ppm).

### Preparation of L9A-P67: 6-[3-(1,3-benzothiazol-2-ylamino)-4-methyl-6,7-dihydro-5H-pyrido[2,3-c]pyridazin-8-yl]-3-[1-[[(5RS,7SR)-3-[2-[4-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-4-methyl-piperazin-4-ium-1-yl]ethoxy]-1-adamantyl]methyl]-5-methyl-pyrazol-4-yl]pyridine-2-carboxylic acid; 2,2,2-trifluoroacetate

Using Method A and P67 as the appropriate payload, the desired product was obtained. HRMS (ESI) [M]+ found=1345.6582 (δ=−6.0 ppm).

### Preparation of L9A-P68: 3-[(5RS,7SR)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]propyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-(3-hydroxypropyl)-methyl-ammonium; 2,2,2-trifluoroacetate

Using Method A and P68 as the appropriate payload, the desired product was obtained. HRMS (ESI) [M]+ found=1360.6941 (δ=−6.0 ppm).

### Preparation of L9C-P69: 6-[3-(1,3-benzothiazol-2-ylamino)-4-methyl-6,7-dihydro-5H-pyrido[2,3-c]pyridazin-8-yl]-3-[1-[[(5RS,7SR)-3-[3-[[(3S)-3,4-dihydroxybutyl]-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl]amino]propyl]-5,7-dimethyl-1-adamantyl]methyl]-5-methyl-pyrazol-4-yl]pyridine-2-carboxylic acid

Using Method C and P69 as the appropriate payload, the desired product was obtained. HRMS (ESI) [M+H]+ found=1420.6913 (δ=3.0 ppm).

### Preparation of L9A-P48: 2-[[(5SR,7RS)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]oxy]ethyl-(carboxymethyl)-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-methyl-ammonium; 2,2,2-trifluoroacetate

Using Method A and P48 as the appropriate payload, the desired product was obtained. HRMS (ESI) [M]+ found=1362.6399 (δ=−3.9 ppm).

### Preparation of L9A-P70: 2-[[(5RS,7SR)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]oxy]ethyl-(2-carboxyethyl)-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-methyl-ammonium; 2,2,2-trifluoroacetate

Using Method A and P70 as the appropriate payload, the desired product was obtained. HRMS (ESI) [M]+ found=1376.6548 (δ=−4.4 ppm).

### Preparation of L9C-P71: 6-[3-(1,3-benzothiazol-2-ylamino)-4-methyl-6,7-dihydro-5H-pyrido[2,3-c]pyridazin-8-yl]-3-[1-[[(5SR,7RS)-3-[2-[2-carboxyethyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl]amino]ethoxy]-5,7-dimethyl-1-adamantyl]methyl]-5-methyl-pyrazol-4-yl]pyridine-2-carboxylic acid

Using Method C and P71 as the appropriate payload, the desired product was obtained. HRMS (ESI) [M+H]⁺ found=1406.6280 (δ=−5.0 ppm).

### Preparation of L9C-P72: 6-[3-(1,3-benzothiazol-2-ylamino)-4-methyl-6,7-dihydro-5H-pyrido[2,3-c]pyridazin-8-yl]-3-[1-[[(5RS,7SR)-3-[3-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-(4-hydroxybutyl)amino]propyl]-5,7-dimethyl-1-adamantyl]methyl]-5-methyl-pyrazol-4-yl]pyridine-2-carboxylic acid

Using Method C and P72 as the appropriate payload, the desired product was obtained. HRMS (ESI) [M+H]⁺ calculated=1404.6927

### Preparation of L9A-P49: 3-[(5SR,7RS)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]propyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-(2-hydroxyethyl)-methyl-ammonium; 2,2,2-trifluoroacetate

Using Method A and P49 as the appropriate payload, the desired product was obtained. HRMS (ESI) [M]+ found=1346.6794 (δ=−5.4 ppm).

### Preparation of L9C-P51: 6-[3-(1,3-benzothiazol-2-ylamino)-4-methyl-6,7-dihydro-5H-pyrido[2,3-c]pyridazin-8-yl]-3-[1-[[(5SR,7RS)-3-[3-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl-(3-methoxypropyl)amino]propyl]-5,7-dimethyl-1-adamantyl]methyl]-5-methyl-pyrazol-4-yl]pyridine-2-carboxylic acid

Using Method C and P51 as the appropriate payload, the desired product was obtained. HRMS (ESI) [M+H]⁺ found=1404.6889 (δ=−2.3 ppm).

### Preparation of L9A-P50: 3-[(5SR,7RS)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]propyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-(3-methoxypropyl)-methyl-ammonium; 2,2,2-trifluoroacetate

Using Method A and P50 as the appropriate payload, the desired product was obtained. HRMS (ESI) [M]⁺ found=1374.7111 (δ=−5.0 ppm).

### Preparation of L9A-P52: 6-[3-(1,3-benzothiazol-2-ylamino)-4-methyl-6,7-dihydro-5H-pyrido[2,3-c]pyridazin-8-yl]-3-[1-[[(5RS,7SR)-3-[3-[1-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]azepan-1-ium-1-yl]propyl]-5,7-dimethyl-1-adamantyl]methyl]-5-methyl-pyrazol-4-yl]pyridine-2-carboxylic acid; 2,2,2-trifluoroacetate

Using Method A and P52 as the appropriate payload, the desired product was obtained. HRMS (ESI) [M]+ found=1370.7281 (δ=3.7 ppm).

### Preparation of L9C-P53: 6-[3-(1,3-benzothiazol-2-ylamino)-4-methyl-6,7-dihydro-5H-pyrido[2,3-c]pyridazin-8-yl]-3-[1-[[(5SR,7RS)-3-[3-[carboxymethyl-[[4-[[(2S)-2-[[(2S)-2-[3- [2-(2,5-dioxopyrrol-1-yl)ethoxy]propanoylamino]-3-methyl-butanoyl]amino]-5-ureido-pentanoyl]amino]phenyl]methoxycarbonyl]amino]propyl]-5,7-dimethyl-1-adamantyl]methyl]-5-methyl-pyrazol-4-yl]pyridine-2-carboxylic acid

Using Method C and P53 as the appropriate payload, the desired product was obtained. HRMS (ESI) [M+H]⁺ found=1390.6301 (δ=−7.2 ppm).

### Preparation of L9A-P55: 3-[(5RS,7SR)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]propyl-(carboxymethyl)-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-methyl-ammonium; 2,2,2-trifluoroacetate

Using Method A and P55 as the appropriate payload, the desired product was obtained. HRMS (ESI) [M]+ found=1360.6561 (δ=−7.2 ppm).

### Preparation of L9C-P54: 6-[3-(1,3-benzothiazol-2-ylamino)-4-methyl-6,7-dihydro-5H-pyrido[2,3-c]pyridazin-8-yl]-3-[1-[[(5SR,7RS)-3-[3-[2-carboxyethyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methoxycarbonyl]amino]propyl]-5,7-dimethyl-1-adamantyl]methyl]-5-methyl-pyrazol-4-yl]pyridine-2-carboxylic acid

Using Method C and P54 as the appropriate payload, the desired product was obtained. HRMS (ESI) [M+H]⁺ found=1404.6464 (δ=−6.7 ppm).

### Preparation of L9C-P47: 6-[3-(1,3-benzothiazol-2-ylamino)-4-methyl-6,7-dihydro-5H-pyrido[2,3-c]pyridazin-8-yl]-3-[1-[[(5SR,7RS)-3-[2-[carboxymethyl-[[4-[[(2S)-2-[[(2S)-2-[3- [2-(2,5-dioxopyrrol-1-yl)ethoxy]propanoylamino]-3-methyl-butanoyl]amino]-5-ureido-pentanoyl]amino]phenyl]methoxycarbonyl]amino]ethoxy]-5,7-dimethyl-1-adamantyl]methyl]-5-methyl-pyrazol-4-yl]pyridine-2-carboxylic acid

Using Method C and P47 as the appropriate payload, the desired product was obtained. HRMS (ESI) [M+H]⁺ found=1392.6186 (δ=−0.6 ppm).

### Preparation of L9A-P56: 3-[(5RS,7SR)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]propyl-(2-carboxyethyl)-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-methyl-ammonium; 2,2,2-trifluoroacetate

Using Method A and P56 as the appropriate payload, the desired product was obtained. HRMS (ESI) [M]+ found=1374.6740 (δ=−5.5 ppm).

### Preparation of L9A-P58: 3-[(5RS,7SR)-3-[[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]propyl-(3-carboxypropyl)-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-methyl-ammonium; 2,2,2-trifluoroacetate

Using Method A and P58 as the appropriate payload, the desired product was obtained. HRMS (ESI) [M]+ found=1388.6891 (δ=−5.9 ppm).

### Preparation of L9A-P57: 2-[[(5SR,7RS)-3-[4-[6-[3-(1,3-benzothiazol-2-ylamino)-4-methyl-6,7-dihydro-5H-pyrido[2,3-c]pyridazin-8-yl]-2-carboxy-3-pyridyl]-5-methyl-pyrazol-1-yl]methyl]-5,7-dimethyl-1-adamantyl]oxy]ethyl-(3-carboxypropyl)-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]phenyl]methyl]-methyl-ammonium; 2,2,2-trifluoroacetate

Using Method A and P57 as the appropriate payload, the desired product was obtained. HRMS (ESI) [M]+ found=1390.6692 (δ=−5.3 ppm).

### Preparation of L9A-P73: (2S)—N-[4-[[1-[2-[[3-[[4-[6-[3-(1,3-benzothiazol-2-ylamino)-4-methyl-6,7-dihydro-5H-pyrido[2,3-c]pyridazin-8-yl]-2-(hydroxymethyl)-3-pyridyl]-5-methyl-pyrazol-1-yl]methyl]-5,7-dimethyl-1-adamantyl]oxy]ethyl]pyrrolidin-1-ium-1-yl]methyl]phenyl]-2-[[(2S)-2-[3-[2-(2,5-dioxopyrrol-1-yl)ethoxy]propanoylamino]-3-methyl-butanoyl]amino]-5-ureido-pentanamide; 2,2,2-trifluoroacetate

Using Method B and P73 as the appropriate payload, the desired product was obtained. HRMS (ESI) [M]+ found=1330.6754 (δ=−12.3 ppm).

### Preparation of L9A-P74: (2S)—N-[4-[[1-[2-[[3-[[4-[6-[3-(1,3-benzothiazol-2-ylamino)-4-methyl-6,7-dihydro-5H-pyrido[2,3-c]pyridazin-8-yl]-2-(pyrrolidine-1-carbonyl)-3-pyridyl]-5-methyl-pyrazol-1-yl]methyl]-5,7-dimethyl-1-adamantyl]oxy]ethyl]pyrrolidin-1-ium-1-yl]methyl]phenyl]-2-[[(2S)-2-[3-[2-(2,5-dioxopyrrol-1-yl)ethoxy]propanoylamino]-3-methyl-butanoyl]amino]-5-ureido-pentanamide; 2,2,2-trifluoroacetate

Using Method B and P74 as the appropriate payload, the desired product was obtained. HRMS (ESI) [M]+ found=1397.7343 (δ=0.2 ppm).

### Preparation of L9A-P75: 6-[3-(1,3-benzothiazol-2-ylamino)-4-methyl-6,7-dihydro-5H-pyrido[2,3-c]pyridazin-8-yl]-3-[1-[[3-[2-[1-[[4-[[(2S)-2-[[(2S)-2-[3-[2-(2,5- dioxopyrrol-1-yl)ethoxy]propanoylamino]-3-methyl-butanoyl]amino]-5-ureido-pentanoyl]amino]phenyl]methyl]pyrrolidin-1-ium-1-yl]ethoxy]-5,7-dimethyl-1-adamantyl]methyl]-5-methyl-pyrazol-4-yl]-N-isopropyl-pyridine-2-carboxamide; 2,2,2-trifluoroacetate

Using Method B and P75 as the appropriate payload, the desired product was obtained. HRMS (ESI) [M]+ found=1385.7328 (δ=−0.8 ppm).

### Preparation of L9A-P76: 6-[3-(1,3-benzothiazol-2-ylamino)-4-methyl-6,7-dihydro-5H-pyrido[2,3-c]pyridazin-8-yl]-3-[1-[[3-[2-[1-[[4-[[(2S)-2-[[(2S)-2-[3-[2-(2,5- dioxopyrrol-1-yl)ethoxy]propanoylamino]-3-methyl-butanoyl]amino]-5-ureido-pentanoyl]amino]phenyl]methyl]pyrrolidin-1-ium-1-yl]ethoxy]-5,7-dimethyl-1-adamantyl]methyl]-5-methyl-pyrazol-4-yl]pyridine-2-carboxamide; 2,2,2-trifluoroacetate

Using Method B and P76 as the appropriate payload, the desired product was obtained. HRMS (ESI) [M]+ found=1343.6874 (δ=0.3 ppm).

### Preparation of L112A-P1: 3-[4-[3-[2-[3-(1,3-benzothiazol-2-ylamino)-4-methyl-6,7-dihydro-5H-pyrido[2,3-c]pyridazin-8-yl]-4-carboxy-thiazol-5-yl]propoxy]-3-fluoro-phenyl]prop-2-ynyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]-2-[3-[2-[2-[2-[2-[2-[2-[2-[2-[2-[2-[2-[2- [2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethox y]ethoxy]ethoxy]ethoxy]ethoxy]ethoxy]ethoxy]ethoxy]ethoxy]ethoxy]ethoxy]ethoxy]etho xy]ethoxy]propyl]phenyl]methyl]-dimethyl-ammonium; 2,2,2-trifluoroacetate

### Step A: 9H-fluoren-9-ylmethyl N-[(1S)-1-[[(1S)-1-[[4-(hydroxymethyl)-3-[3-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethox y]ethoxy]ethoxy]ethoxy]ethoxy]ethoxy]ethoxy]ethoxy]ethoxy]ethoxy]ethoxy]ethoxy]etho xy]ethoxy]propyl]phenyl]carbamoyl]-4-ureido-butyl]carbamoyl]-2-methyl-propyl]carbamate

The title compound was synthesized according to the experimental procedure described in WO2020/236817A₂, Preparation of L26-P1 using 2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]e thoxy]ethoxy]ethanol as the starting material. ¹H NMR (400 MHz, dmso-d₆): δ 9.88 (s, 1H), 8.07 (d, 1H), 7.89 (d, 2H), 7.72-7.76 (m, 2H), 7.37-7.45 (m, 5H), 7.30-7.34 (m, 2H), 7.25 (d, 1H), 5.95 (t, 1H), 5.38 (s, 2H), 4.95 (t, 1H), 4.45 (d, 2H), 4.38-4.42 (m, 1H), 4.20-4.32 (m, 3H), 3.90-3.94 (m, 1H), 3.45-3.55 (m, 94H), 3.38-3.43 (m, 4H), 3.23 (s, 3H), 2.89-3.03 (m, 2H), 2.56-2.62 (m, 2H), 1.94-2.04 (m, 1H), 1.54-1.76 (m, 4H), 1.29-1.49 (m, 2H), 0.84-0.89 (m, 6H). UPLC-MS: MS (ESI) m/z [M/2+Na]+ found=888.

### Step B: 9H-fluoren-9-ylmethyl N-[(1S)-1-[[(1S)-1-[[4-(chloromethyl)-3-[3-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]propyl]phenyl]carbamoyl]-4-ureido-butyl]carbamoyl]-2-methyl-propyl]carbamate

To the product from Step A (50 mg, 0.0288 mmol) in THF (0.7 ml) was added equivalent amount of thionyl chloride (0.35 M solution in THF) in every 10 min until no starting material was observed. The mixture was concentrated, and the crude product was used in the next step without further purification.

### Step C: 9H-fluoren-9-ylmethyl N-[(1S)-1-[[(1S)-1-[[4-(iodomethyl)-3-[3-[2-[2-[2-[2-[2- [2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]propyl]phenyl]carbamoyl]-4-ureido-butyl]carbamoyl]-2-methyl-propyl]carbamate

After stirring the mixture of the product from Step B (44 mg, 0.025 mmol) and sodium iodide (2 eq) in butan-2-one (30 mL/mmol) for 5 h, the reaction was concentrated and the crude product was used in the next step without further purification.

### Step D: 3-[4-[3-[2-[3-(1,3-benzothiazol-2-ylamino)-4-methyl-6,7-dihydro-5H-pyrido[2,3-c]pyridazin-8-yl]-4-carboxy-thiazol-5-yl]propoxy]-3-fluoro-phenyl]prop-2-ynyl-[[4-[[(2S)-2-[[(2S)-2-(9H-fluoren-9-ylmethoxycarbonylamino)-3-methyl-butanoyl]amino]-5-ureido-pentanoyl]amino]-2-[3-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethox y]ethoxy]ethoxy]ethoxy]ethoxy]ethoxy]ethoxy]ethoxy]ethoxy]ethoxy]ethoxy]ethoxy]etho xy]ethoxy]propyl]phenyl]methyl]-dimethyl-ammonium

After stirring the mixture of payload P1 (15 mg, 0.023 mmol), the product from Step C (46.18 mg, 0.025 mmol) and DIPEA (5 eq) in DMF (0.7 mL) for 44 h, the crude product was concentrated and used in the next step without further purification.

### Step E: [4-[[(2S)-2-[[(2S)-2-amino-3-methyl-butanoyl]amino]-5-ureido-pentanoyl]amino]-2-[3-[2-[2-[2-[2-[2-[2-[2-[2-[2-[2-[2-[2-[2-[2-[2-[2-[2- [2-[2-[2-[2-[2-[2-(2-methoxyethoxy)ethoxy]ethoxy]ethoxy]ethoxy]ethoxy]ethoxy]ethoxy]ethoxy]ethoxy]ethox y]ethoxy]ethoxy]ethoxy]ethoxy]ethoxy]ethoxy]ethoxy]ethoxy]ethoxy]ethoxy]ethoxy]etho xy]ethoxy]propyl]phenyl]methyl-[3-[4-[3-[2-[3-(1,3-benzothiazol-2-ylamino)-4-methyl-6,7-dihydro-5H-pyrido[2,3-c]pyridazin-8-yl]-4-carboxy-thiazol-5-yl]propoxy]-3-fluoro-phenyl]prop-2-ynyl]-dimethyl-ammonium

After stirring the mixture of the product from Step D (54 mg, 0.023 mmol) and N-ethylethanamine (10 eq) in DMF (0.7 mL) for 1 h, the crude product was purified by preparative HPLC to give the desired compound (22 mg). UPLC-MS: MS (ESI) m/z [(M+2)/2] found=1075.

### Step F: 3-[4-[3-[2-[3-(1,3-benzothiazol-2-ylamino)-4-methyl-6,7-dihydro-5H-pyrido[2,3-c]pyridazin-8-yl]-4-carboxy-thiazol-5-yl]propoxy]-3-fluoro-phenyl]prop-2-ynyl-[[4-[[(2S)-2-[[(2S)-2-[3-[2-(2,5-dioxopyrrol-1-yl)ethoxy]propanoylamino]-3-methyl-butanoyl]amino]-5-ureido-pentanoyl]amino]-2-[3-[2-[2-[2-[2-[2-[2-[2-[2-[2-[2-[2-[2-[2-[2-[2-[2-[2-[2-[2-[2-[2-[2-[2-(2-methoxyethoxy)ethoxy]ethoxy]ethoxy]ethoxy]ethoxy]ethoxy]ethoxy]ethoxy]ethoxy]ethox y]ethoxy]ethoxy]ethoxy]ethoxy]ethoxy]ethoxy]ethoxy]ethoxy]ethoxy]ethoxy]ethoxy]etho xy]ethoxy]propyl]phenyl]methyl]-dimethyl-ammonium;2,2,2-trifluoroacetate

After stirring the mixture of the product from Step E (22 mg, 0.0097 mmol), DIEA (2 eq) and (2,5-dioxopyrrolidin-1-yl) 3-[2-(2,5-dioxopyrrol-1-yl)ethoxy]propanoate (1.1 eq) in DMF (0.3 mL) for 15 h, the crude product was purified using preparative HPLC and using the TFA method to give L112A-P1 (5.5 mg). HR-ESI+: m/z [M−CF3COO]+ found=2344.

### Example 4. Synthesis and Characterization of Additional Linkers, Linker-Payloads, and Precursors Thereof.

Exemplary linkers, linker-payloads, and precursors thereof were synthesized using exemplary methods described in this example.

### Synthesis of 2-(bromomethyl)-4-nitrobenzoic acid

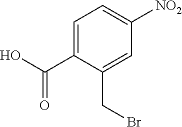 [SMILES: O=C(O)c1ccc([N+](=O)[O-])cc1CBr]

To a stirred solution of 2-methyl-4-nitrobenzoic acid (300 g, 1.5371 mol) in CCI4 (3000 mL) was added NBS (300.93 g, 1.6908 mol) and AlBN (37.86 g, 0.2305 mol) at RT. The reaction mixture was stirred at 80° C. for 16 h. Reaction mixture was monitored by TLC analysis. The reaction mixture was diluted with sat. NaHCO₃ solution (2 L) and extracted with ethyl acetate (2×2 L). The combined organic layer was dried over anhydrous sodium sulfate and concentrated under reduced pressure. The crude compound was purified by column chromatography on silica gel using 2-3% of ethyl acetate in petroleum-ether as an eluent and 2-(bromomethyl)-4-nitrobenzoic acid was obtained. ¹H NMR (400 MHz, CDCl₃): δ 8.35 (d, J=2.0 Hz, 1H), 8.20 (q, J=8.8, 2.4 Hz, 1H), 8.12 (d, J=8.8 Hz, 1H), 4.97 (s, 2H), 4.00 (s, 3H).

### Synthesis of 4-nitro-2-((prop-2-yn-1-yloxy)methyl)benzoic acid

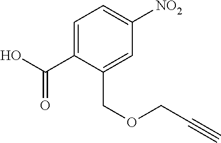 [SMILES: C#CCOCc1cc([N+](=O)[O-])ccc1C(=O)O]

To the mixture of 2-(bromomethyl)-4-nitrobenzoic acid (250 g, 0.9122 mol) in MeCN (5000 mL) was added prop-2-yn-1-ol (255.68 g, 265.50 mL, 4.5609 mol, d=0.963 g/mL) and Cs₂CO₃ (743.03 g, 2.2805 mol) at RT. The resulting mixture was heated to 80° C. for 16 h. The reaction mixture was filtered through celite pad washed with ethyl acetate (2 L). The filtrate was concentrated under reduced pressure. The obtained crude compound was added sat. NaHCO₃ solution (1 L) and the aqueous layer was acidified to pH 2 by using 2N HCl (2 L). After filtration vacuum drying 4-nitro-2-((prop-2-yn-1-yloxy)methyl)benzoic acid was obtained. ¹H NMR (400 MHz, DMSO): δ 13.61 (brs, 1H), 8.37 (d, J=2.4 Hz, 1H), 8.23 (dd, J=2.4, 8.4 Hz, 1H), 8.10 (d, J=8.8 Hz, 1H), 4.95 (s, 2H), 4.37 (d, J=2.4 Hz, 2H), 3.52 (t, J=2.4 Hz, 1H)

### Synthesis of methyl 4-nitro-2-((prop-2-yn-1-yloxy)methyl)benzoate

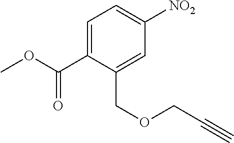 [SMILES: C#CCOCc1cc([N+](=O)[O-])ccc1C(=O)OC]

To a stirred solution of 4-nitro-2-((prop-2-yn-1-yloxy)methyl)benzoic acid (130 g, 0.5527 mol) in MeOH (1300 mL) was added SOCl₂ (526.08 g, 320.78 mL, 4.4219 mol, d=1.64 g/mL) slowly at 0° C. The reaction stirred at 70° C. for 4 h. The reaction solvent was evaporated under reduced pressure. The obtained residue was dissolved in ethyl acetate (1000 mL) and washed with sat. NaHCO₃ (600 mL), water (500 mL) and brine solution (500 mL). The separated organic layer was dried over sodium sulphate, filtered and evaporated under reduced pressure to yield methyl 4-nitro-2-((prop-2-yn-1-yloxy)methyl)benzoate. ¹H NMR (400 MHz, CDCl₃): δ 8.56 (t, J=0.8 Hz, 1H), 8.18-8.09 (m, 2H), 5.03 (s, 2H), 4.35 (d, J=2.4 Hz, 2H), 3.96 (s, 3H), 2.49 (t, J=2.4 Hz, 1H).

### Synthesis of methyl 4-amino-2-((prop-2-yn-1-yloxy)methyl)benzoate

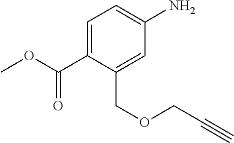 [SMILES: C#CCOCc1cc(N)ccc1C(=O)OC]

To a solution of methyl 4-nitro-2-((prop-2-yn-1-yloxy)methyl)benzoate (110 g, 0.4414 mol) in a mixture of EtOH (1100 mL) and H₂O (550 mL) was added Fe powder (197.21 g, 3.5310 mol) and NH₄Cl (188.88 g, 3.5310 mol) at RT. The resulting mixture was heated at 80° C. for 16 h. The reaction mixture was cooled to RT and filtered through Celite® and washed with ethyl acetate (2 L). The filtrate was concentrated under reduced pressure up to half of the volume. To the residue, ethyl acetate (1.5 L) was added and separated the two layers and the aqueous layer was extracted with ethyl acetate (2 L). The combined organic layer was dried over anhydrous sodium sulfate and concentrated under reduced pressure to obtain crude product. Purification by SiO₂ column chromatography (15-20% of ethyl acetate in petroleum-ether) yielded methyl 4-amino-2-((prop-2-yn-1-yloxy)methyl)benzoate. ¹H NMR (400 MHz, CDCl₃): δ 7.67 (d, J=8.8 Hz, 1H), 6.78 (t, J=1.6 Hz, 1H), 6.48 (q, J=8.4, 2.4 Hz, 1H), 4.79 (s, 2H), 4.25 (d, J=2.4 Hz, 2H), 3.70 (d, J=4.0 Hz, 3H), 3.42 (t, J=2.4 Hz, 1H).

### Synthesis of (4-amino-2-((prop-2-yn-1-yloxy)methyl)phenyl)methanol

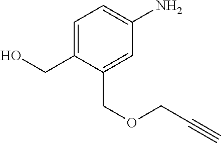

To a stirred solution of THF (1000 mL) was added LiAlH₄ (1 M in THF)(21.23 g, 798.2 mmol, 798.2 mL) slowly at 0° C. A solution of methyl 4-amino-2-((prop-2-yn-1-yloxy)methyl)benzoate (70 g, 319.3 mmol) in THF (800 mL) was added slowly at 0° C. The reaction was stirred at RT for 4 h. The reaction mixture was cooled to 0° C., then was added water (22 mL) very slowly and followed by the addition of 20% NaOH (22 mL) and water (66 mL). The reaction mixture was stirred at 0° C. for 30 min. Anhydrous sodium sulfate was added to absorb excess of water. The mixture was filtered through Celite®. The filter cake was washed with ethyl acetate (1000 mL) and 10% MeOH/DCM (500 mL). The filtrate was concentrated under reduced pressure. The resulting crude compound was purified by SiO₂ column chromatography (35-40% of ethyl acetate in petroleum-ether as an eluent) to give yield (4-amino-2-((prop-2-yn-1-yloxy)methyl)phenyl)methanol. ¹H NMR (400 MHz, CDCl₃): δ 6.98 (d, J=8.0 Hz, 1H), 6.56 (d, J=2.4 Hz, 1H), 6.43 (dd, J=2.4, 8.0 Hz, 1H), 4.98 (s, 2H), 4.64 (t, J=5.2 Hz, 1H), 4.47 (s, 2H), 4.34 (d, J=5.6 Hz, 2H), 4.15 (d, J=2.4 Hz, 2H), 3.46 (t, J=2.4 Hz, 1H).

### Synthesis of (9H-fluoren-9-yl)methyl (S)-(1-((4-(hydroxymethyl)-3-((prop-2-yn-1-yloxy)methyl)phenyl)amino)-1-oxo-5-ureidopentan-2-yl)carbamate

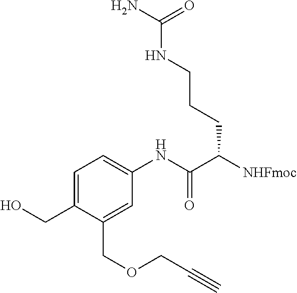

To a solution of (4-amino-2-((prop-2-yn-1-yloxy)methyl)phenyl)methanol (1.92 g, 10.04 mmol, 1.0 equiv.), (9H-fluoren-9-yl)methyl (S)-(1-amino-1-oxo-5-ureidopentan-2-yl)carbamate (3.99 g, 10.04 mmol, 1.0 equiv.), and (1-[bis(dimethylamino)methylene]-1H-1,2,3-triazolo[4,5-b]pyridinium 3-oxid hexafluorophosphate (4.20 g, 11.04 mmol, 1.1 equiv.) in DMF (10 mL) was added N,N-diisopropylethylamine (2.62 mL, 15.06 mmol, 1.5 equiv.). After stirring at ambient temperature for 1 h, the mixture was poured into water (200 mL). The resulting solids were filtered, rinsed with water, and dried under vacuum, and (9H-fluoren-9-yl)methyl (S)-(1-((4-(hydroxymethyl)-3-((prop-2-yn-1-yloxy)methyl)phenyl)amino)-1-oxo-5-ureidopentan-2-yl)carbamate was obtained. LCMS: MH+=571.5; Rt=0.93 min (2 min acidic method).

### Synthesis of (S)-2-amino-N-(4-(hydroxymethyl)-3-((prop-2-yn-1-yloxy)methyl)phenyl)-5-ureidopentanamide

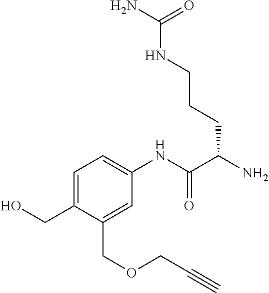 [SMILES: C#CCOCc1cc(NC(=O)[C@@H](N)CCCNC(N)=O)ccc1CO]

To (9H-fluoren-9-yl)methyl (S)-(1-((4-(hydroxymethyl)-3-((prop-2-yn-1-yloxy)methyl)phenyl)amino)-1-oxo-5-ureidopentan-2-yl)carbamate (6.08 g, 10.65 mmol, 1.0 equiv.) was added dimethylamine (2 M in THF, 21.31 mL, 42.62 mmol, 4 equiv.). After stirring at ambient temperature for 1.5 hours, the supernatant solution was decanted from the gum-like residue that had formed. The residue was triturated with ether (3×50 mL) and the resulting solids were filtered, washed with ether, and dried under vacuum. (S)-2-amino-N-(4-(hydroxymethyl)-3-((prop-2-yn-1-yloxy)methyl)phenyl)-5-ureidopentanamide was obtained. LCMS: MH+349.3; Rt=0.42 min (2 min acidic method).

### Synthesis of tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate

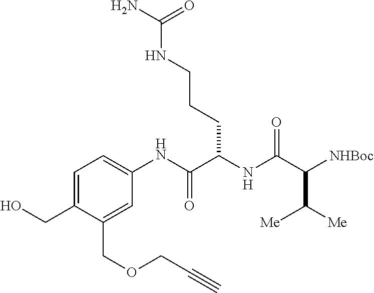 [SMILES: C#CCOCc1cc(NC(=O)[C@H](CCCNC(N)=O)NC(=O)[C@@H](NC(=O)OC(C)(C)C)C(C)C)ccc1CO]

To a solution of (S)-2-amino-N-(4-(hydroxymethyl)-3-((prop-2-yn-1-yloxy)methyl)phenyl)-5-ureidopentanamide (3.50 g, 10.04 mmol, 1.0 equiv.), (tert-butoxycarbonyl)-L-valine (2.62 g, 12.05 mmol, 1.2 equiv.), and (1-[bis(dimethylamino)methylene]-1H-1,2,3-triazolo[4,5-b]pyridinium 3-oxid hexafluorophosphate (4.58 g, 12.05 mmol, 1.2 equiv.) in DMF (10 mL) was added N,N-diisopropylethylamine (3.50 mL, 20.08 mmol, 2.0 equiv.). After stirring at ambient temperature for 2 h, the mixture was poured into water (200 mL) and the resulting suspension was extracted with EtOAc (3×100 mL). The combined organic layers were dried over sodium sulfate and concentrated under vacuum. After purification by ISCO SiO₂ chromatography (0-20% methanol/dichloromethane), tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate was obtained. ¹H NMR (400 MHz, DMSO-d₆) δ 10.00 (s, 1H), 7.96 (d, J=7.7 Hz, 1H), 7.55 (dq, J=4.9, 2.2 Hz, 2H, aryl), 7.32 (d, J=8.9 Hz, 1H, aryl), 6.76 (d, J=8.9 Hz, 1H), 5.95 (t, J=5.8 Hz, 1H), 5.38 (s, 2H), 5.01 (t, J=5.5 Hz, 1H), 4.54 (s, 2H), 4.45 (dd, J=25.2, 5.3 Hz, 3H), 4.20 (d, J=2.4 Hz, 2H), 3.83 (dd, J=8.9, 6.7 Hz, 1H), 3.49 (t, J=2.4 Hz, 1H), 2.97 (dh, J=26.0, 6.5 Hz, 2H), 1.96 (h, J=6.6 Hz, 1H), 1.74-1.50 (m, 2H), 1.39 (m, 11H), 0.84 (dd, J=16.2, 6.7 Hz, 6H). LCMS: M+Na 570.5; Rt=0.79 min (2 min acidic method).

### Synthesis of tert-butyl ((S)-1-(((S)-1-((4-(chloromethyl)-3-((prop-2-yn-1-yloxy)methyl)phenyl)amino)-1-oxo-5-ureidopentan-2-yl)amino)-3-methyl-1-oxobutan-2-yl)carbamate

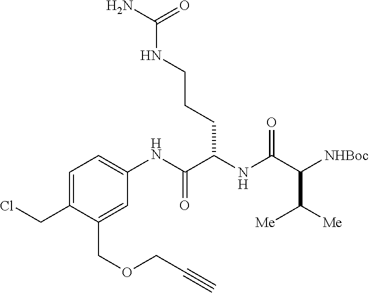 [SMILES: C#CCOCc1cc(NC(=O)[C@H](CCCNC(N)=O)NC(=O)[C@@H](NC(=O)OC(C)(C)C)C(C)C)ccc1CCl]

To a solution of tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate (2.00 grams, 3.65 mmol, 1.0 equiv.) in acetonitrile (13.3 mL) at 0° C. was added thionyl chloride (0.53 mL, 7.30 mmol, 2.0 equiv.). After stirring in the ice bath for one hour the solution was diluted with water (40 mL) and the resulting white precipitate was collected by filtration, air drying and drying under high vacuum to yield tert-butyl ((S)-1-(((S)-1-((4-(chloromethyl)-3-((prop-2-yn-1-yloxy)methyl)phenyl)amino)-1-oxo-5-ureidopentan-2-yl)amino)-3-methyl-1-oxobutan-2-yl)carbamate. LCMS: M+Na 588.5; Rt=2.17 min (5 min acidic method).

### Synthesis of 2-(((tert-butyldiphenylsilyl)oxy)methyl)-5-nitrobenzoic acid

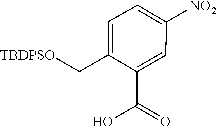

To a solution of 6-nitroisobenzofuran-1 (3H)-one (90 g, 502.43 mmol, 1.00 equiv.) in MeOH (1000 mL) and KOH (28.19 g, 502.43 mmol, 1.00 equiv.) in H₂O (150 mL) was added. The brown mixture was stirred at 25° C. for 1.5 h. The brown mixture was concentrated under reduced pressure to give a residue and dissolved in DCM (2000 mL). To the mixture was added tert-Butyldiphenylchlorosilane (296.91 g, 1.08 mol, 277.49 mL, 2.15 equiv.) and imidazole (171.03 g, 2.51 mol, 5.00 equiv.) and stirred at 25° C. for 12 h. The mixture was concentrated under reduced pressure to give a residue. The residue was purified by silica gel chromatography (Petroleum ether/Ethyl acetate=1/0, 1/1) and 2-(((tert-butyldiphenylsilyl)oxy)methyl)-5-nitrobenzoic acid was obtained as a white solid. ¹H NMR (400 MHz, METHANOL-d4) δ ppm 1.13 (s, 9H) 5.26 (s, 2H) 7.34-7.48 (m, 6H) 7.68 (br d, J=8 Hz, 4H) 8.24 (br d, J=8 Hz, 1H) 8.46 (br d, J=8 Hz, 1H) 8.74 (s, 1H).

### Synthesis of (2-(((tert-butyldiphenylsilyl)oxy)methyl)-5-nitrophenyl)methanol

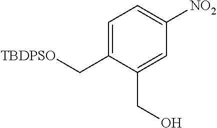

To a mixture of 2-(((tert-butyldiphenylsilyl)oxy)methyl)-5-nitrobenzoic acid (41 g, 94.14 mmol, 1 equiv.) in THF (205 mL) was added BH3. THF (1 M, 470.68 mL, 5 equiv.). The yellow mixture was stirred at 60° C. for 2 h. The mixture was added MeOH (400 mL), and concentrated under reduced pressure to give a residue. Then addition of H₂O (200 mL) and DCM (300 mL), extracted with DCM (3×200 mL), washed with brine (300 mL), dried over anhydrous MgSO₄, filtered, and concentrated under reduced pressure to give a residue. The residue was purified by silica gel chromatography (Petroleum ether/Ethyl acetate=1/0, 1/1). (2-(((tert-butyldiphenylsilyl)oxy)methyl)-5-nitrophenyl)methanol was obtained as a white solid. ¹H NMR (400 MHz, METHANOL-d4) δ ppm 1.10 (s, 9H) 4.58 (s, 2H) 4.89 (s, 2H) 7.32-7.51 (m, 6H) 7.68 (dd, J=8, 1.38 Hz, 4H) 7.76 (d, J=8 Hz, 1H) 8.15 (dd, J=8 2.26 Hz, 1H) 8.30 (d, J=2 Hz, 1 H).

### Synthesis of 2-(((tert-butyldiphenylsilyl)oxy)methyl)-5-nitrobenzaldehyde

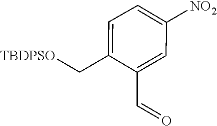

To a solution of (2-(((tert-butyldiphenylsilyl)oxy)methyl)-5-nitrophenyl)methanol (34 g, 80.65 mmol, 1 equiv.) in DCM (450 mL) was added MnO₂ (56.09 g, 645.22 mmol, 8 equiv.). The black mixture was stirred at 25° C. for 36 h. The mixture was added MeOH (400 mL), and concentrated under reduced pressure to give a residue. Then addition of H₂O (200 mL) and DCM (300 mL), extracted with DCM (3×200 mL), washed with brine (300 mL), dried over anhydrous MgSO₄, filtered, and concentrated under reduced pressure to give a residue. The residue was purified by silica gel chromatography (CH₂Cl₂=100%). 2-(((tert-butyldiphenylsilyl)oxy)methyl)-5-nitrobenzaldehyde was obtained as a white solid. ¹H NMR (400 MHz, CHLOROFORM-d) 0 ppm 1.14 (s, 9H) 5.26 (s, 2H) 7.34-7.53 (m, 6H) 7.60-7.73 (m, 4H) 8.13 (d, J=8 Hz, 1H) 8.48 (dd, J=8, 2.51 Hz, 1H) 8.67 (d, J=2 Hz, 1H) 10.16 (s, 1H).

### Synthesis of N-(2-(((tert-butyldiphenylsilyl)oxy)methyl)-5-nitrobenzyl)prop-2-yn-1-amine

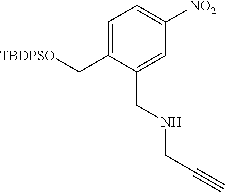

To a solution of 2-(((tert-butyldiphenylsilyl)oxy)methyl)-5-nitrobenzaldehyde (12.6 g, 30.03 mmol, 1 equiv.) in DCM (130 mL) was added prop-2-yn-1-amine (4.14 g, 75.08 mmol, 4.81 mL, 2.5 equiv.) and MgSO₄ (36.15 g, 300.33 mmol, 10 equiv.) then the suspension mixture was stirred at 25° C. for 24 hr. Taking a little reaction solution and treating with NaBH4, the TLC showed one new spot was formed. The reaction mixture was filtered and concentrated under reduced pressure to give a residue. (E)-N-[[2-[[tert-butyl(diphenyl)silyl]oxymethyl]-5-nitro-phenyl]methyl]prop-2-yn-1-imine was obtained as a yellow solid. ¹H NMR (400 MHz, CHLOROFORM-d) 0 ppm 1.11 (s, 9H) 2.48 (t, J=2.38 Hz, 1H) 4.52 (t, J=2.13 Hz, 2H) 5.09 (s, 2H) 7.35-7.49 (m, 6H) 7.63-7.72 (m, 4H) 7.79 (d, J=8.53 Hz, 1H) 8.25 (dd, J=8.53, 2.51 Hz, 1H) 8.68 (d, J=2.26 Hz, 1H) 8.84 (t, J=1.88 Hz, 1H).

(E)-N-[[2-[[tert-butyl(diphenyl)silyl]oxymethyl]-5-nitro-phenyl]methyl]prop-2-yn-1-imine (12 g, 26.28 mmol, 1 equiv.) was dissolved in MeOH (100 mL) and THF (50 mL), then NaBH4 (1.49 g, 39.42 mmol, 1.5 equiv.) was added and the yellow mixture was stirred at −20° C. for 2 hr. LCMS showed desired compound was detected. The reaction mixture was quenched by addition MeOH (200 mL) at −20° C., and then concentrated under reduced pressure to give a residue. The residue was dissolved with EtOAc (500 mL) washed with brine (150 mL), dried over anhydrous Na₂SO₄, filtered and concentrated under reduced pressure to give a residue. The residue was purified by flash silica gel chromatography (Eluent of 0-10% Ethyl acetate/Petroleum ether gradient). N-(2-(((tert-butyldiphenylsilyl)oxy)methyl)-5-nitrobenzyl)prop-2-yn-1-amine was obtained as a pale yellow oil. ¹H NMR (400 MHz, CHLOROFORM-d) 0 ppm 1.12 (s, 9H) 2.13 (t, J=2.38 Hz, 1H) 3.33 (d, J=2.51 Hz, 2H) 3.80 (s, 2H) 4.93 (s, 2H) 7.36-7.49 (m, 6H) 7.69 (dd, J=7.91, 1.38 Hz, 4H) 7.77 (d, J=8.53 Hz, 1H) 8.16 (dd, J=8.41, 2.38 Hz, 1H) 8.24 (d, J=2.26 Hz, 1H).

### Synthesis of (9H-fluoren-9-yl)methyl (2-(((tert-butyldiphenylsilyl)oxy)methyl)-5-nitrobenzyl)(prop-2-yn-1-yl)carbamate

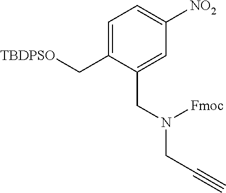

To a solution of N-(2-(((tert-butyldiphenylsilyl)oxy)methyl)-5-nitrobenzyl)prop-2-yn-1-amine (9 g, 19.62 mmol, 1 equiv.) and Fmoc-OSu (7.28 g, 21.59 mmol, 1.1 equiv.) in dioxane (90 mL) was added sat. NaHCO₃ (90 mL) and the white suspension was stirred at 20° C. for 12 h. The reaction mixture was diluted with H₂O (150 mL) and extracted with EtOAc (150 mL×2). The combined organic layers were washed with brine (200 mL), dried over anhydrous Na₂SO₄, filtered and concentrated under reduced pressure to give a residue. The residue was purified by flash silica gel chromatography (Eluent of 0-30% Ethyl acetate/Petroleum ether). (9H-fluoren-9-yl)methyl (2-(((tert-butyldiphenylsilyl)oxy)methyl)-5-nitrobenzyl)(prop-2-yn-1-yl)carbamate (7.7 g, 11.08 mmol, 56.48% yield, 98% purity) was obtained as a white solid. ¹H NMR (400 MHz, CHLOROFORM-d) δ ppm 1.12 (s, 9H) 2.17 (br d, J=14.31 Hz, 1H) 3.87-4.97 (m, 9H) 6.98-8.28 (m, 21H).

### Synthesis of (9H-fluoren-9-yl)methyl (5-amino-2-(((tert-butyldiphenylsilyl)oxy)methyl)benzyl)(prop-2-yn-1-yl)carbamate

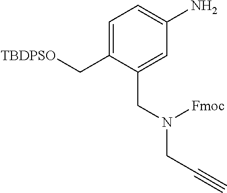

To an ice bath cooled solution of (9H-fluoren-9-yl)methyl (2-(((tert-butyldiphenylsilyl)oxy)methyl)-5-nitrobenzyl)(prop-2-yn-1-yl)carbamate (5.0 g, 7.34 mmol, 1.0 equiv.) in 10% AcOH/CH₂Cl₂ (100 mL) was added Zn (7.20 g, 110 mmol, 15 equiv.). The ice bath was removed, and the resulting mixture stirred for 2 hours at which time it was filtered through a pad of Celite®. The volatiles were removed in vacuo and the residue was dissolved in EtOAc, was washed with NaHCO₃(sat.), NaCl(sat.), dried over MgSO₄, filtered, concentrated and after ISCO SiO₂ chromatography (0-75% EtOAc/Heptane)(9H-fluoren-9-yl)methyl (5-amino-2-(((tert-butyldiphenylsilyl)oxy)methyl)benzyl)(prop-2-yn-1-yl)carbamate was obtained. LCMS: MH+=651.6; Rt=3.77 min (5 min acidic method).

### Synthesis of (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate

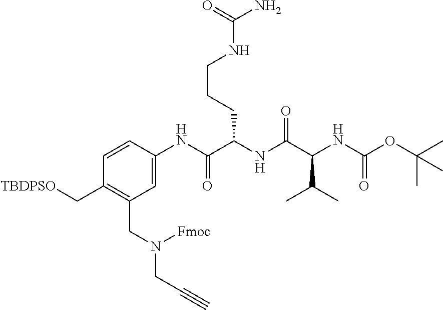

To (9H-fluoren-9-yl)methyl (5-amino-2-(((tert-butyldiphenylsilyl)oxy)methyl)benzyl)(prop-2-yn-1-yl)carbamate (2.99 g, 4.59 mmol, 1.0 equiv.) and(S)-2-((S)-2-((tert-butoxycarbonyl)amino)-3-methylbutanamido)-5-ureidopentanoic acid (1.72 g, 4.59 mmol, 1.0 equiv.) in CH₂Cl₂ (40 mL) was added ethyl 2-ethoxyquinoline-1 (2H)-carboxylate (2.27 g, 9.18 mmol, 2.0 equiv.). After stirring for 10 min, MeOH (1 mL) was added and the solution became homogeneous. The reaction was stirred for 16 h, the volatiles were removed in vacuo and after purification by ISCO SiO₂ chromatography (0-15% MeOH/CH₂Cl₂)(9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate was obtained. LCMS: MH+=1008.8; Rt=3.77 min (5 min acidic method).

### Synthesis of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate

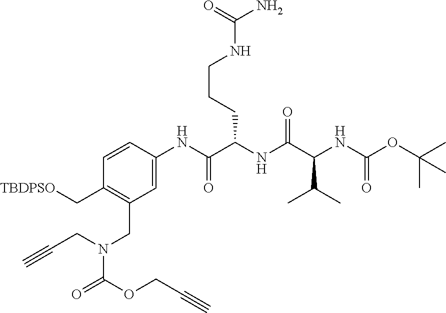

To (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate (1.60 g, 1.588 mmol, 1.0 equiv.) was added 2M dimethylamine in MeOH (30 mL, 60 mmol, 37 equiv.) and THF (10 mL). After standing for 3 h, the volatiles were removed in vacuo and the residue was triturated with Et₂O to remove FMOC deprotection byproducts. To the resulting solid was added CH₂Cl₂ (16 mL) and pyridine (4 mL) and to the heterogeneous solution was added propargyl chloroformate (155 μL, 1.588 mmol, 1.0 equiv.). After stirring for 30 minutes additional propargyl chloroformate (155 μL, 1.588 mmol, 1.0 equiv.) was added. After stirring for an additional 20 min, MeOH (1 mL) was added to quench remaining chloroformate and the volatiles were removed in vacuo. Upon purification by ISCO SiO₂ chromatography (0-15% MeOH/CH₂Cl₂)prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate was obtained. LCMS: MH+=867.8; Rt=3.40 min (5 min acidic method).

### Synthesis of prop-2-yn-1-yl (5-((S)-2-((S)-2-((tert-butoxycarbonyl)amino)-3-methylbutanamido)-5-ureidopentanamido)-2-(hydroxymethyl)benzyl)(prop-2-yn-1-yl)carbamate

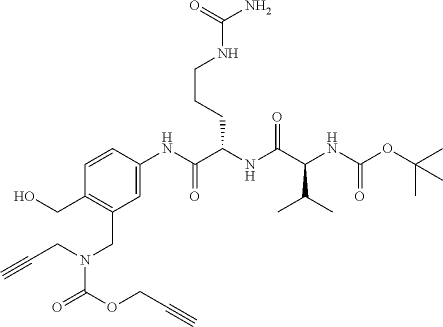 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1CO]

To a solution of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(prop-2-yn-1-yl)carbamate (984 mg, 1.135 mmol, 1.0 equiv.) in THF (7.5 mL) was added 1.0 M TBAF in THF (2.27 mL, 2.27 mmol, 2.0 equiv.). After standing for 6 h, the volatiles were removed in vacuo, the residue was purified by ISCO SiO₂ chromatography (0-40% MeOH/CH2Cl2) and prop-2-yn-1-yl (5-((S)-2-((S)-2-((tert-butoxycarbonyl)amino)-3-methylbutanamido)-5-ureidopentanamido)-2-(hydroxymethyl)benzyl)(prop-2-yn-1-yl)carbamate was obtained. LCMS: MH+=629.6; Rt=1.74 min (5 min acidic method).

### Synthesis of prop-2-yn-1-yl (5-((S)-2-((S)-2-((tert-butoxycarbonyl)amino)-3-methylbutanamido)-5-ureidopentanamido)-2-(chloromethyl)benzyl)(prop-2-yn-1-yl)carbamate

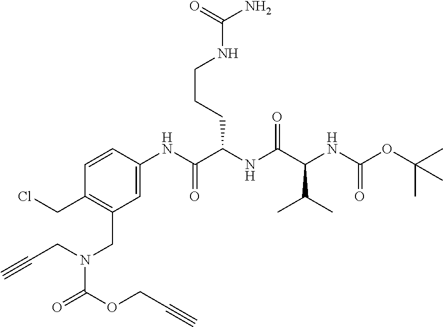 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1CCl]

To prop-2-yn-1-yl (5-((S)-2-((S)-2-((tert-butoxycarbonyl)amino)-3-methylbutanamido)-5-ureidopentanamido)-2-(hydroxymethyl)benzyl)(prop-2-yn-1-yl)carbamate (205 mg, 0.326 mmol, 1.0 equiv.) in CH₂Cl₂ (10 mL) was added pyridine (158 μL, 1.96 mmol, 5 equiv.). The heterogeneous mixture was cooled in a 0° C. ice bath and thionyl chloride (71 μL, 0.98 mmol, 3 equiv.). After stirring in the ice bath for 3 hours the reaction was directly purified by ISCO SiO₂ chromatography (0-30% MeOH/CH₂Cl₂) and prop-2-yn-1-yl (5-((S)-2-((S)-2-((tert-butoxycarbonyl)amino)-3-methylbutanamido)-5-ureidopentanamido)-2-(chloromethyl)benzyl)(prop-2-yn-1-yl)carbamate was obtained. LCMS: MH+=647.6; Rt=2.54 min (5 min acidic method).

### Synthesis of 2-(hydroxymethyl)-N-methyl-5-nitrobenzamide

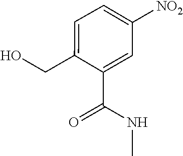

To a stirred suspension of 6-nitroisobenzofuran-1 (3H)-one (500 g, 2.79 mol) in MeOH (1500 mL) was added MeNH₂ (3.00 kg, 29.94 mol, 600 mL, 31.0% purity) at 25° C. and stirred for 1 h. The solid was filtered and washed with water twice (600 mL) and dried under high vacuum to get a residue. The product 2-(hydroxymethyl)-N-methyl-5-nitrobenzamide was obtained as white solid. LCMS: Rt=0.537 min, MS m/z=193.2. ¹H NMR: 400 MHz DMSO δ 8.57 (br d, J=4.4 Hz, 1H), 8.31 (dd, J=2.4, 8.6 Hz, 1H), 8.21 (d, J=2.4 Hz, 1H), 7.86 (d, J=8.8 Hz, 1H), 5.54 (t, J=5.6 Hz, 1H), 4.72 (d, J=5.5 Hz, 2H), 2.78 (d, J=4.4 Hz, 3H).

### Synthesis of (2-((methylamino)methyl)-4-nitrophenyl)methanol

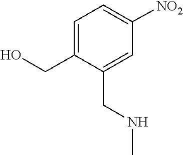

To a solution of 2-(hydroxymethyl)-N-methyl-5-nitrobenzamide (560 g, 2.66 mol) in THF (5000 mL) was cooled to 0° C., then added BH3-Me2S (506 g, 6.66 mol)(2.0 M in THF) dropwise for 60 min and heated to 70° C. for 5 h. LCMS showed the starting material was consumed. After completion, 4M HCl (1200 mL) in Methanol was added to reaction mixture at 0° C. and heated at 65° C. for 8 h. The reaction mixture was cooled to 0° C., the solid was filtered and concentrated under reduce pressure. The product (2-((methylamino)methyl)-4-nitrophenyl)methanol (520 g) was obtained as a white solid. LCMS: Rt=0.742 min, MS m/z=197.1 [M+H]⁺. ¹H NMR: 400 MHz DMSO δ 9.25 (br s, 2H), 8.37 (d, J=2.4 Hz, 1H), 8.14 (dd, J=2.4, 8.5 Hz, 1H), 7.63 (d, J=8.4 Hz, 1H), 5.72 (br s, 1H), 4.65 (s, 2H), 4.15 (br s, 2H), 2.55-2.45 (m, 3H)

### Synthesis of 1-(2-(((tert-butyldiphenylsilyl)oxy)methyl)-5-nitrophenyl)-N-methylmethanamine

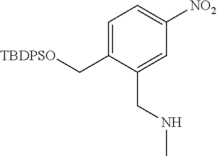

To a solution of (2-((methylamino)methyl)-4-nitrophenyl)methanol (520 g, 2.65 mol) and imidazole (721 g, 10.6 mol) in DCM (2600 mL) was cooled to 0° C. was added TBDPS-CL (1.09 kg, 3.98 mol, 1.02 L) drop wise and stirred for 2 h. The mixture was poured in ice cold water (1000 mL) and extracted with ethyl acetate. The combined organic layers were washed with brine, dried over Na₂SO₄, filtered and evaporated under vacuum to give crude product. The crude product was purified by chromatography on a silica gel eluted with Ethyl acetate: Petroleum ether (from 10/1 to 1) to give a residue. The product 1-(2-(((tert-butyldiphenylsilyl)oxy)methyl)-5-nitrophenyl)-N-methylmethanamine was obtained as yellow liquid. LCMS: product: Rt=0.910 min, MS m/z=435.2 [M+H]⁺. ¹H NMR: 400 MHz CDCl3 δ 8.23 (d, J=2.4 Hz, 1H), 8.15 (dd, J=2.4, 8.4 Hz, 1H), 7.76 (d, J=8.4 Hz, 1H), 7.71-7.66 (m, 4H), 7.50-7.37 (m, 6H), 4.88 (s, 2H), 3.65 (s, 2H), 2.39 (s, 3H), 1.12 (s, 9H)

### Synthesis of (9H-fluoren-9-yl)methyl (2-(((tert-butyldiphenylsilyl)oxy)methyl)-5-nitrobenzyl)(methyl)carbamate

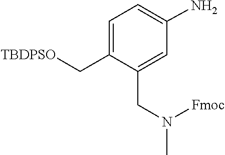

To a solution of 1-(2-(((tert-butyldiphenylsilyl)oxy)methyl)-5-nitrophenyl)-N-methylmethanamine (400 g, 920.3 mmol) in THF (4000 mL) was added Fmoc-OSu (341.5 g, 1.01 mol) and Et₃N (186.2 g, 1.84 mol, 256.2 mL), the mixture was stirred at 25° C. for 1 h. The mixture was poured into water (1600 mL) and extracted with ethyl acetate (1000 mL×2). The combined organic layers were washed with brine, dried over Na₂SO₄, filtered and evaporated under vacuum to give crude product. The crude product was purified by chromatography on a silica gel eluted with petroleum ether:ethyl acetate (from 1/0 to 1/1) to give (9H-fluoren-9-yl)methyl (2-(((tert-butyldiphenylsilyl)oxy)methyl)-5-nitrobenzyl)(methyl)carbamate as white solid. LCMS: Rt=0.931 min, MS m/z=657.2 [M+H]⁺. ¹H NMR: EW16000-26-P1A, 400 MHz CDCl3 δ 8.21-7.96 (m, 1H), 7.87-7.68 (m, 3H), 7.68-7.62 (m, 4H), 7.62-7.47 (m, 2H), 7.47-7.28 (m, 9H), 7.26-7.05 (m, 2H), 4.81 (br s, 1H), 4.62-4.37 (m, 4H), 4.31-4.19 (m, 1H), 4.08-3.95 (m, 1H), 2.87 (br d, J=5.2 Hz, 3H), 1.12 (s, 9H).

### Synthesis of (9H-fluoren-9-yl)methyl (5-amino-2-(((tert-butyldiphenylsilyl)oxy)methyl)benzyl)(methyl)carbamate

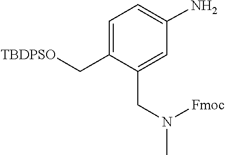

A solution of (9H-fluoren-9-yl)methyl (2-(((tert-butyldiphenylsilyl)oxy)methyl)-5-nitrobenzyl)(methyl)carbamate (3.0 g, 4.57 mmol, 1.0 equiv.) in MeOH (90 mL) and EtOAc (30 mL) was degassed and purged to a balloon of N₂ via three way stopcock. After repeating degas/N₂ purge 2×, 10% Pd/C deGussa type (0.486 g, 0.457 mmol, 0.1 equiv.) was added. The resulting mixture was degassed and purged to a balloon of 2 H₂ via three-way stopcock. After repeating degas/H₂ purge 2×, the reaction stirred under the balloon pressure of H₂ for 4 hours. The reaction was degassed and purged to N₂, filtered through a pad of celite eluting further with MeOH. After removal of the volatiles in vacuo and pumping on high vacuum (9H-fluoren-9-yl)methyl (5-amino-2-(((tert-butyldiphenylsilyl)oxy)methyl)benzyl)(methyl)carbamate was obtained. LCMS: MH+=627.7; Rt=1.59 min (2 min acidic method).

### Synthesis of (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate

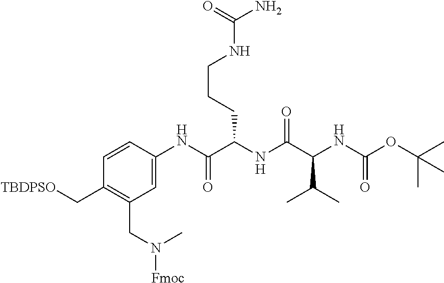

To (9H-fluoren-9-yl)methyl (5-amino-2-(((tert-butyldiphenylsilyl)oxy)methyl)benzyl)(methyl)carbamate (2.86 g, 4.56 mmol, 1.0 equiv.) and(S)-2-((S)-2-((tert-butoxycarbonyl)amino)-3-methylbutanamido)-5-ureidopentanoic acid (1.71 g, 4.56 mmol, 1.0 equiv.) in 2:1 CH₂Cl₂/MeOH (60 mL) was added ethyl 2-ethoxyquinoline-1 (2H)-carboxylate (2.256 g, 9.12 mmol, 2.0 equiv.). The homogeneous solution was stirred for 16 hours at which time additional(S)-2-((S)-2-((tert-butoxycarbonyl)amino)-3-methylbutanamido)-5-ureidopentanoic acid (0.340 g, 0.2 equiv.) and ethyl 2-ethoxyquinoline-1 (2H)-carboxylate (0.452 g, 0.4 equiv.) were add to drive the reaction to completion. After stirring for an additional 5 hours the volatiles were removed in vacuo and after purification by ISCO SiO₂ chromatography (0-5% MeOH/CH₂Cl₂) (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate was obtained. LCMS: MH+=984.1; Rt=1.54 min (2 min acidic method).

### Synthesis of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate

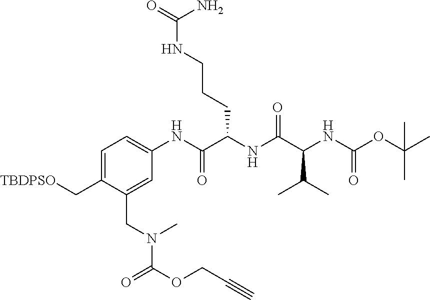

To (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate (2.05 g, 2.085 mmol, 1.0 equiv.) in THF (10 mL) was added 2.0 M dimethyl amine in MeOH (10.42 mL, 20.85 mmol, 10 equiv.). After stirring for 16 hours the volatiles were removed in vacuo. The residue was dissolved in CH₂Cl₂ (20 mL) and DIEA (0.533 mL, 4.17 mmol, 2 equiv.) and propargyl chloroformate (0.264 mL, 2.71 mmol, 1.3 equiv.) were added. After stirring at RT for 16 hours the reaction was diluted with CH₂Cl₂ (20 mL), was washed with NaHCO₃ (sat.), NaCl(sat.), dried over MgSO₄, filtered, concentrated and purified by ISCO SiO₂ chromatography (0-15% MeOH/CH₂Cl₂) to yield prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate. LCMS: MH+=843.8; Rt=1.35 min (2 min acidic method).

### Synthesis of prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate

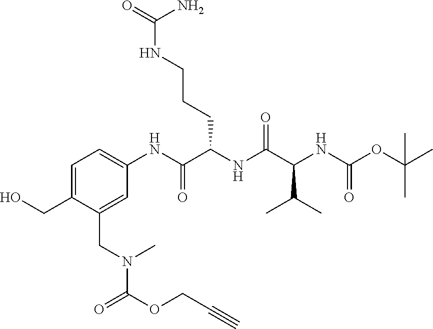 [SMILES: C#CCOC(=O)N(C)Cc1cc(NC(=O)[C@H](CCCNC(N)=O)NC(=O)[C@@H](NC(=O)OC(C)(C)C)C(C)C)ccc1CO]

To a 0° C. solution of prop-2-yn-1-yl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate (1.6 g, 1.90 mmol, 1.0 equiv.) in THF (10.0 mL) was added 1.0 M TBAF in THF (3.80 mL, 3.80 mmol, 2.0 equiv.). After warming to RT and stirring for 16 h the volatiles were removed in vacuo, the residue was dissolved in EtOAc, was washed with NaHCO₃ (sat.), with NaCl(sat.), dried over MgSO₄, filtered, concentrated and the residue was purified by ISCO SiO₂ chromatography (0-30% MeOH/CH2Cl2) to yield prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate. LCMS: MH+=605.7; Rt=0.81 min (2 min acidic method).

### Synthesis of prop-2-yn-1-yl (5-((S)-2-((S)-2-((tert-butoxycarbonyl)amino)-3-methylbutanamido)-5-ureidopentanamido)-2-(chloromethyl)benzyl)(methyl)carbamate

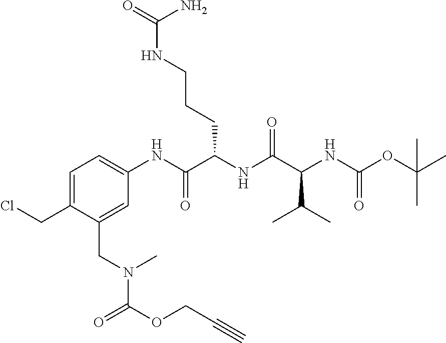 [SMILES: C#CCOC(=O)N(C)Cc1cc(NC(=O)[C@H](CCCNC(N)=O)NC(=O)[C@@H](NC(=O)OC(C)(C)C)C(C)C)ccc1CCl]

To prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate (350 mg, 0.579 mmol, 1.0 equiv.) in CH₂Cl₂ (10 mL) was added pyridine (0.278 mL, 3.47 mmol, 6 equiv.). The heterogeneous mixture was cooled in a 0° C. ice bath and thionyl chloride (0.126 mL, 1.73 mmol, 3 equiv.). After stirring in the ice bath for 3 h, the reaction was purified by ISCO SiO₂ chromatography (0-30% MeOH/CH₂Cl₂) and prop-2-yn-1-yl (5-((S)-2-((S)-2-((tert-butoxycarbonyl)amino)-3-methylbutanamido)-5-ureidopentanamido)-2-(chloromethyl)benzyl)(prop-2-yn-1-yl)carbamate was obtained. LCMS: MH+=623.7; Rt=2.19 min (5 min acidic method).

### Synthesis of (9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate

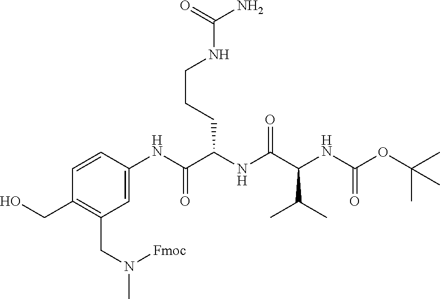

To (9H-fluoren-9-yl)methyl (5-((S)-2-((S)-2-((tert-butoxycarbonyl)amino)-3-methylbutanamido)-5-ureidopentanamido)-2-(((tert-butyldiphenylsilyl)oxy)methyl)benzyl)(methyl)carbamate (2.6 g, 2.64 mmol, 1.0 equiv.) dissolved in THF (20 mL) was added acetic acid (0.757 mL, 13.22 mmol, 5.0 equiv.) and 1.0 M TBAF in THF (2.91 mL, 2.91 mmol, 1.1 equiv.). The solution was stirred for 72 hours at which time the volatiles were removed in vacuo. After purification by ISCO SiO₂ chromatography (0-30% MeOH/CH₂Cl₂)(9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate was obtained. LCMS: MH+=745.5; Rt=1.07 min (2 min acidic method).

### Synthesis of (9H-fluoren-9-yl)methyl (5-((S)-2-((S)-2-((tert-butoxycarbonyl)amino)-3-methylbutanamido)-5-ureidopentanamido)-2-(chloromethyl)benzyl)(methyl)carbamate

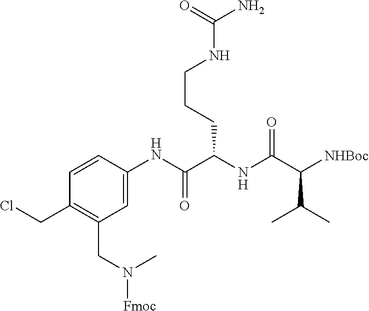

To (9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate (1.0 gram, 1.342 mmol) in THF (20 mL) was added NaHCO₃ (677 mg, 8.05 mmol)(6 eq), then cooled to 0° C. in ice-water bath, followed by adding thionyl chloride (0.245 mL, 3.36 mmol)(2.5 eq) slowly. The mixture was stirred at 0° C. for 15 min, then at RT for 1 h. The reaction was partitioned between EtOAc and NaHCO₃ (sat.), separated, washed with NaCl(sat.), dried over MgSO₄ and the volatiles were removed in vacuo. The residue was purified by ISCO SiO₂ chromatography (0-30% iPrOH/CH₂Cl₂) to yield (9H-fluoren-9-yl)methyl (5-((S)-2-((S)-2-((tert-butoxycarbonyl)amino)-3-methylbutanamido)-5-ureidopentanamido)-2-(chloromethyl)benzyl)(methyl)carbamate was obtained. LCMS: MH+=763.2; Rt=1.18 min (2 min acidic method).

### General Procedure 1

### Synthesis of prop-2-yn-1-yl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate

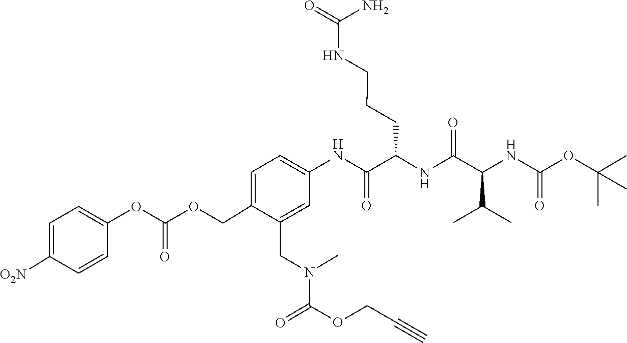 [SMILES: C#CCOC(=O)N(C)Cc1cc(NC(=O)[C@H](CCCNC(N)=O)NC(=O)[C@@H](NC(=O)OC(C)(C)C)C(C)C)ccc1COC(=O)Oc1ccc([N+](=O)[O-])cc1]

A solution of prop-2-yn-1-yl (5-((S)-2-((S)-2-((tert-butoxycarbonyl)amino)-3-methylbutanamido)-5-ureidopentanamido)-2-(hydroxymethyl)benzyl)(methyl)carbamate (249 mg, 0.412 mmol) and bis(4-nitrophenyl) carbonate (356 mg, 1.24 mmol, 3.0 equiv.) in DMF (2 mL) was swirled until homogeneous and sat for 16 hours. The solution was diluted with DMSO (6 mL) and was purified by RP-HPLC ISCO gold chromatography (10-100% MeCN/H₂O, no modifier). Upon lyophilization, prop-2-yn-1-yl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate was obtained. LC/MS MH+=770.7, Rt=2.45 min (5 min acidic method).

### Synthesis of tert-butyl ((S)-1-(((S)-1-((4-(hydroxymethyl)phenyl)amino)-1-oxo-5-ureidopentan-2-yl)amino)-3-methyl-1-oxobutan-2-yl)carbamate

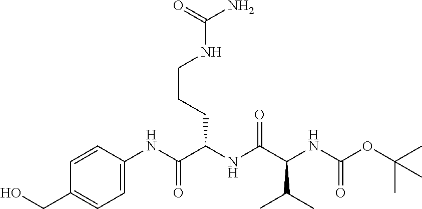 [SMILES: CC(C)[C@H](NC(=O)OC(C)(C)C)C(=O)N[C@@H](CCCNC(N)=O)C(=O)Nc1ccc(CO)cc1]

To a suspension of (4-aminophenyl)methanol (450.0 mg, 3.65 mmol) and(S)-2-((S)-2-((tert-butoxycarbonyl)amino)-3-methylbutanamido)-5-ureidopentanoic acid (1368.0 mg, 3.65 mmol, 1.0 equiv.) in DCM (4.0 mL) was added EEDQ (2259.0 mg, 9.13 mmol, 2.5 equiv.). The mixture was stirred for 16 hours at RT, after which the reaction was purified by ISCO SiO₂ chromatography (0-30% MeOH/CH₂Cl₂) and tert-butyl ((S)-1-(((S)-1-((4-(hydroxymethyl)phenyl)amino)-1-oxo-5-ureidopentan-2-yl)amino)-3-methyl-1-oxobutan-2-yl)carbamate was obtained. LC/MS MH+=480.6, Rt=0.75 min (2 min acidic method). ¹H NMR (400 MHz, DMSO-d₆) δ 9.97 (s, 1H), 7.96 (d, J=7.7 Hz, 1H), 7.60-7.48 (m, 2H), 7.29-7.19 (m, 2H), 6.76 (d, J=8.9 Hz, 1H), 5.96 (t, J=5.8 Hz, 1H), 5.40 (s, 2H), 5.09 (t, J=5.7 Hz, 1H), 4.43 (d, J=5.7 Hz, 3H), 3.83 (dd, J=8.9, 6.7 Hz, 1H), 2.98 (dp, J=30.3, 6.6 Hz, 2H), 1.95 (p, J=6.7 Hz, 1H), 1.80-1.54 (m, 2H), 1.38 (s, 11H), 0.84 (dd, J=15.9, 6.8 Hz, 6H).

### Synthesis of tert-butyl ((S)-1-(((S)-1-((4-(chloromethyl)phenyl)amino)-1-oxo-5-ureidopentan-2-yl)amino)-3-methyl-1-oxobutan-2-yl)carbamate

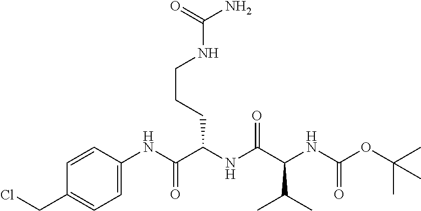 [SMILES: CC(C)[C@H](NC(=O)OC(C)(C)C)C(=O)N[C@@H](CCCNC(N)=O)C(=O)Nc1ccc(CCl)cc1]

To tert-butyl ((S)-1-(((S)-1-((4-(hydroxymethyl)phenyl)amino)-1-oxo-5-ureidopentan-2-yl)amino)-3-methyl-1-oxobutan-2-yl)carbamate (500.0 mg, 1.043 mmol) in DCM (20.0 mL) was added pyridine (0.506 mL, 6.26 mmol, 6.0 equiv.). The heterogeneous mixture was cooled in an 0° C. ice bath and thionyl chloride (0.228 mL, 3.13 mmol, 3 equiv.) was added. After stirring in the ice bath for 4 hours, the mixture was warmed up to RT for 15 min. The reaction was purified by ISCO SiO₂ chromatography (0-30% MeOH/CH2Cl2) and tert-butyl ((S)-1-(((S)-1-((4-(chloromethyl)phenyl)amino)-1-oxo-5-ureidopentan-2-yl)amino)-3-methyl-1-oxobutan-2-yl)carbamate was obtained. LC/MS MH+=498.1, Rt=2.02 min (5 min acidic method).

### Synthesis of (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate

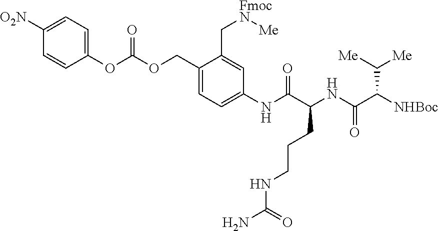

Following GENERAL PROCEDURE 1 with (9H-fluoren-9-yl)methyl (5-((S)-2-((S)-2-((tert-butoxycarbonyl)amino)-3-methylbutanamido)-5-ureidopentanamido)-2-(hydroxymethyl)benzyl)(methyl)carbamate (100.0 mg, 0.134 mmol), (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate was obtained. LC/MS MH+=910.5, Rt=1.24 min (2 min acidic method). ¹H NMR (400 MHz, DMSO-d₆) δ 10.19 (s, 1H), 8.26 (s, 2H), 8.00 (d, J=7.7 Hz, 1H), 7.93-7.58 (m, 4H), 7.42 (td, J=33.3, 32.9, 13.8 Hz, 9H), 7.14 (s, 1H), 6.72 (d, J=9.0 Hz, 1H), 6.01 (s, 1H), 5.27 (d, J=23.7 Hz, 2H), 4.58 (s, 2H), 4.48-4.13 (m, 4H), 3.89-3.78 (m, 1H), 2.92 (t, J=35.0 Hz, 5H), 2.00-1.86 (m, 1H), 1.54 (s, 3H), 1.37 (m, 11H, incl. Boc), 0.82 (dd, J=15.4, 6.7 Hz, 6H).

### Synthesis of (9H-fluoren-9-yl)methyl((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate

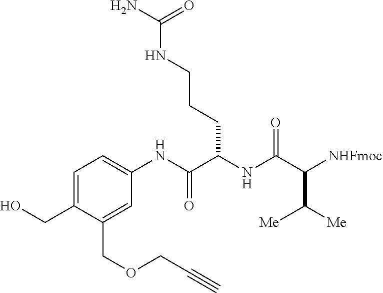

To a solution of (S)-2-amino-N-(4-(hydroxymethyl)-3-((prop-2-yn-1-yloxy)methyl)phenyl)-5-ureidopentanamide (3.64 g, 10.45 mmol), (S)-2-((((9H-fluoren-9-yl)methoxy)carbonyl)amino)-3-methylbutanoic acid (3.55 g, 10.54 mmol, 1.0 equiv.) and 1-((dimethylamino)(dimethyliminio)methyl)-1H-[1,2,3]triazolo[4,5-b]pyridine 3-oxide hexafluorophosphate (V)(3.97 g, 10.54 mmol, 1.0 equiv.) in DMF (10.0 mL) was added DIPEA (3.64 mL, 20.90 mmol, 2.0 equiv.). The mixture was stirred for 45 min. at RT. Diluted with 100 mL water, stirred for 5 min. and filtered the precipitate which was dried under reduced vacuo. Upon drying, (9H-fluoren-9-yl)methyl((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate was obtained. LC/MS MH+=670.3, Rt=0.96 min (2 min acidic method).

### Synthesis of (9H-fluoren-9-yl)methyl((S)-3-methyl-1-(((S)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate

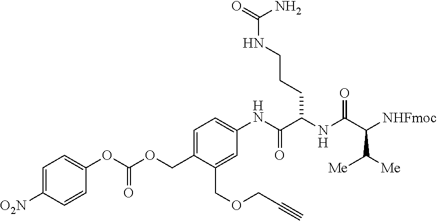

Following GENERAL PROCEDURE 1 with (9H-fluoren-9-yl)methyl((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate (200.0 mg, 0.299 mmol), (9H-fluoren-9-yl)methyl((S)-3-methyl-1-(((S)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate was obtained. LC/MS MH+=835.7, Rt=1.19 min (2 min acidic method).

### Synthesis of tert-butyl((R)-3-methyl-1-(((R)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate

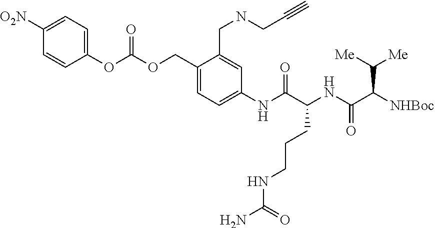 [SMILES: C#CCNCc1cc(NC(=O)[C@@H](CCCNC(N)=O)NC(=O)[C@H](NC(=O)OC(C)(C)C)C(C)C)ccc1COC(=O)Oc1ccc([N+](=O)[O-])cc1]

Following GENERAL PROCEDURE 1 with tert-butyl ((S)-1-(((S)-1-((4-(hydroxymethyl)-3-((prop-2-yn-1-yloxy)methyl)phenyl)amino)-1-oxo-5-ureidopentan-2-yl)amino)-3-methyl-1-oxobutan-2-yl)carbamate (200.0 mg, 0.365 mmol), tert-butyl((R)-3-methyl-1-(((R)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate was obtained. LC/MS MH+=713.6, Rt=1.08 min (2 min acidic method).

### Synthesis of prop-2-yn-1-yl 5-((S)-2-((S)-2-((((9H-fluoren-9-yl)methoxy)carbonyl)amino)-3-methylbutanamido)-5-ureidopentanamido)-2-(hydroxymethyl)benzyl(methyl)carbamate

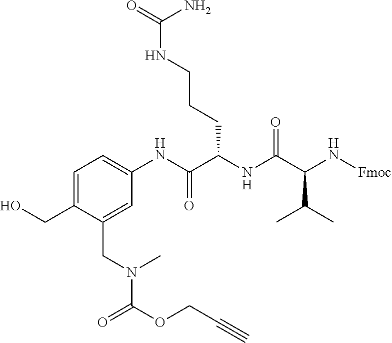

To a solution of prop-2-yn-1-yl 5-((S)-2-((S)-2-((tert-butoxycarbonyl)amino)-3-methylbutanamido)-5-ureidopentanamido)-2-(hydroxymethyl)benzyl(methyl)carbamate (48.0 mg, 0.079 mmol) in DCM (1.0 mL) at 0° C. was added TFA (0.2 mL). The mixture was stirred for 1 hour at this temperature. Afterwards the solvents were removed under vacuo. The residue was dissolved in DMF (1.0 mL), followed by adding DIPEA (0.138 mL, 0.794 mmol, 10 equiv.) and (9H-fluoren-9-yl)methyl (2,5-dioxopyrrolidin-1-yl) carbonate (40.2 mg, 0.119 mmol, 1.5 equiv.). The mixture was stirred for 18 hours at RT. Reaction was purified by RP-HPLC ISCO gold chromatography (0-100% MeCN/H2O, no modifier). Upon lyophilization, prop-2-yn-1-yl 5-((S)-2-((S)-2-((((9H-fluoren-9-yl)methoxy)carbonyl)amino)-3-methylbutanamido)-5-ureidopentanamido)-2-(hydroxymethyl)benzyl(methyl)carbamate was obtained. LC/MS MH+=727.3, Rt=2.28 min (5 min acidic method). ¹H NMR (400 MHz, DMSO-d₆) δ 10.01 (s, 1H), 8.09 (d, J=7.6 Hz, 1H), 7.89 (d, 2H), 7.74 (t, J=8.2 Hz, 2H), 7.62 (s, 1H), 7.45-7.36 (m, 3H), 7.35-7.15 (m, 4H), 5.95 (t, J=5.9 Hz, 1H), 5.36 (s, 2H), 5.03 (s, 1H), 4.70 (d, J=14.8 Hz, 2H), 4.54-4.36 (m, 5H), 4.35-4.19 (m, 3H), 3.96-3.87 (m, 1H), 3.50 (d, J=26.0 Hz, 1H), 2.97 (dp, J=20.1, 6.6 Hz, 2H), 2.82 (s, 3H), 1.98 (q, J=6.8 Hz, 1H), 1.73-1.50 (m, 2H), 1.51-1.30 (m, 2H), 0.86 (dd, J=10.2, 6.7 Hz, 6H).

### Synthesis of prop-2-yn-1-yl 5-((S)-2-((S)-2-((((9H-fluoren-9-yl)methoxy)carbonyl)amino)-3-methylbutanamido)-5-ureidopentanamido)-2-((((4-nitrophenoxy)carbonyl)oxy)methyl)benzyl(methyl)carbamate

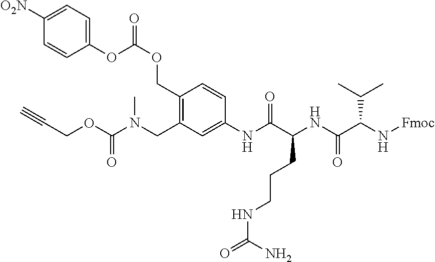

Following GENERAL PROCEDURE 1 with prop-2-yn-1-yl 5-((S)-2-((S)-2-((((9H-fluoren-9-yl)methoxy)carbonyl)amino)-3-methylbutanamido)-5-ureidopentanamido)-2-(hydroxymethyl)benzyl(methyl)carbamate (77.6 mg, 0.107 mmol), prop-2-yn-1-yl 5-((S)-2-((S)-2-((((9H-fluoren-9-yl)methoxy)carbonyl)amino)-3-methylbutanamido)-5-ureidopentanamido)-2-((((4-nitrophenoxy)carbonyl)oxy)methyl)benzyl(methyl)carbamate was obtained. LC/MS MH+=892.4, Rt=1.14 min (2 min acidic method).

### Synthesis of prop-2-yn-1-yl 5-((S)-2-((S)-2-((tert-butoxycarbonyl)amino)-3-methylbutanamido)-5-ureidopentanamido)-2-((((4-nitrophenoxy)carbonyl)oxy)methyl)benzyl(prop-2-yn-1-yl)carbamate

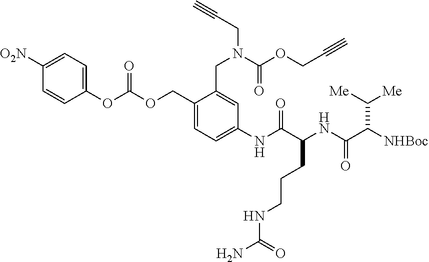 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1COC(=O)Oc1ccc([N+](=O)[O-])cc1]

Following GENERAL PROCEDURE 1 with prop-2-yn-1-yl 5-((S)-2-((S)-2-((tert-butoxycarbonyl)amino)-3-methylbutanamido)-5-ureidopentanamido)-2-(hydroxymethyl)benzyl(prop-2-yn-1-yl)carbamate (250.0 mg, 0.398 mmol), prop-2-yn-1-yl 5-((S)-2-((S)-2-((tert-butoxycarbonyl)amino)-3-methylbutanamido)-5-ureidopentanamido)-2-((((4-nitrophenoxy)carbonyl)oxy)methyl)benzyl(prop-2-yn-1-yl)carbamate was obtained. LC/MS MH+=794.9, Rt=1.07 min (2 min acidic method).

### Synthesis of prop-2-yn-1-yl 2-(((tert-butyldiphenylsilyl)oxy)methyl)-5-nitrobenzyl(prop-2-yn-1-yl)carbamate

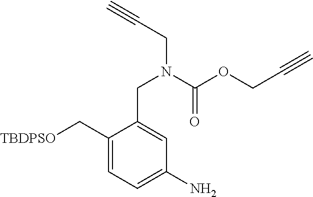

To a solution of N-(2-(((tert-butyldiphenylsilyl)oxy)methyl)-5-nitrobenzyl)prop-2-yn-1-amine (1.348 g, 2.94 mmol) in DCM (10.0 mL) was added pyridine (2.0 mL) followed by prop-2-yn-1-yl carbonochloridate (0.574 mL, 5.88 mmol, 2.0 equiv.) and the mixture was stirred for 30 min. at RT. Reaction was quenched with MeOH, diluted with CH₂Cl₂ (20 mL), then washed with water, NaCl(sat.), dried over Na₂SO₄, filtered, concentrated and purified by ISCO SiO₂ chromatography (0-50% EtOAc/heptane), prop-2-yn-1-yl 2-(((tert-butyldiphenylsilyl)oxy)methyl)-5-nitrobenzyl(prop-2-yn-1-yl)carbamate was obtained. LC/MS MH+=541.6, Rt=1.47 min (2 min acidic method). ¹H NMR (400 MHz, Chloroform-d) δ 8.18 (dd, J=8.4, 2.4 Hz, 1H, Ar), 8.10 (d, J=2.3 Hz, 1H, Ar), 7.72-7.63 (m, 4H, Ph), 7.54-7.35 (m, 7H, Ph+Ar), 4.86 (s, 2H), 4.80-4.53 (m, 4H), 4.02 (d, J=22.3 Hz, 2H), 2.76 (d, J=4.7 Hz, 1H), 2.17 (t, J=2.4 Hz, 1H), 1.13 (d, J=3.1 Hz, 9H).

### Synthesis of prop-2-yn-1-yl 5-amino-2-(((tert-butyldiphenylsilyl)oxy)methyl)benzyl(prop-2-yn-1-yl)carbamate

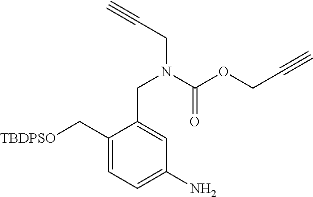

To a solution of prop-2-yn-1-yl 2-(((tert-butyldiphenylsilyl)oxy)methyl)-5-nitrobenzyl(prop-2-yn-1-yl)carbamate (1.66 g, 2.07 mmol) in DCM (9.0 mL) and AcOH (1.0 mL) at 0° C. was added zinc (3.01 g, 46.1 mmol, 15.0 equiv.) and the mixture was stirred for 40 min. at this temperature. Reaction was filtered through celite and rinsed with DCM. Filtrate was washed with NaHCO₃ (sat.), water and NaCl(sat.), dried over Na₂SO₄, filtered, concentrated and purified by ISCO SiO₂ chromatography (0-100% EtOAc/heptane), prop-2-yn-1-yl 5-amino-2-(((tert-butyldiphenylsilyl)oxy)methyl)benzyl(prop-2-yn-1-yl)carbamate was obtained. LC/MS M+Na=533.2, Rt=1.35 min (2 min acidic method).

### Synthesis of prop-2-yn-1-yl 5-((S)-2-((S)-2-((((9H-fluoren-9-yl)methoxy)carbonyl)amino)-3-methylbutanamido)-5-ureidopentanamido)-2-(((tert-butyldiphenylsilyl)oxy)methyl)benzyl(prop-2-yn-1-yl)carbamate

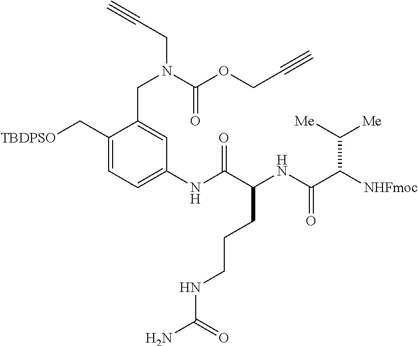

Suspended prop-2-yn-1-yl 5-amino-2-(((tert-butyldiphenylsilyl)oxy)methyl)benzyl(prop-2-yn-1-yl)carbamate (1.19 g, 2.33 mmol) and(S)-2-((S)-2-((((9H-fluoren-9-yl)methoxy)carbonyl)amino)-3-methylbutanamido)-5-ureidopentanoic acid (1.157 g, 2.33 mmol, 1.0 equiv.) in DCM (10.0 mL) and MeOH (5.0 mL), added EEDQ (0.691 g, 2.80 mmol, 1.2 equiv.) and stirred for 3 hours at RT. Solvents were removed in vacuo, residue dissolved in DMSO (3.0 mL) and purified by RP-HPLC ISCO gold chromatography (0-100% MeCN/H2O, 0.05% TFA modifier). Upon lyophilization, prop-2-yn-1-yl 5-((S)-2-((S)-2-((((9H-fluoren-9-yl)methoxy)carbonyl)amino)-3-methylbutanamido)-5-ureidopentanamido)-2-(((tert-butyldiphenylsilyl)oxy)methyl)benzyl(prop-2-yn-1-yl)carbamate was obtained. LC/MS M+H=990.0, Rt-1.47 min (2 min acidic method).

### Synthesis of prop-2-yn-1-yl 5-((S)-2-((S)-2-((((9H-fluoren-9-yl)methoxy)carbonyl)amino)-3-methylbutanamido)-5-ureidopentanamido)-2-(hydroxymethyl)benzyl(prop-2-yn-1-yl)carbamate

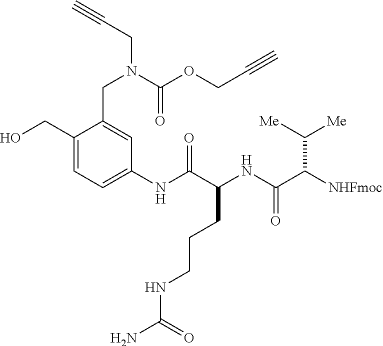

To a solution of prop-2-yn-1-yl 5-((S)-2-((S)-2-((((9H-fluoren-9-yl)methoxy)carbonyl)amino)-3-methylbutanamido)-5-ureidopentanamido)-2-(((tert-butyldiphenylsilyl)oxy)methyl)benzyl(prop-2-yn-1-yl)carbamate (732.0 mg, 0.740 mmol) in THF (5.0 mL) was added acetic acid (0.127 mL, 2.220 mmol, 3.0 equiv.) and 1.0 M TBAF in THF (1.48 mL, 1.480 mmol, 2.0 equiv.). The mixture was stirred at RT for 20 hours. LCMS indicated some start material left. Added 1.0 M TBAF in THF (0.75 mL, 0.750 mmol, 1.0 equiv.) and stirred at RT for 20 hours. Solvent was removed in vacuo, the material was purified by ISCO SiO₂ chromatography (0-50% MeOH/CH₂Cl₂) and prop-2-yn-1-yl 5-((S)-2-((S)-2-((((9H-fluoren-9-yl)methoxy)carbonyl)amino)-3-methylbutanamido)-5-ureidopentanamido)-2-(hydroxymethyl)benzyl(prop-2-yn-1-yl)carbamate was obtained. LC/MS M+H=751.6, Rt=0.99 min (2 min acidic method).

### Synthesis of prop-2-yn-1-yl 5-((S)-2-((S)-2-((((9H-fluoren-9-yl)methoxy)carbonyl)amino)-3-methylbutanamido)-5-ureidopentanamido)-2-((((4-nitrophenoxy)carbonyl)oxy)methyl)benzyl(prop-2-yn-1-yl)carbamate

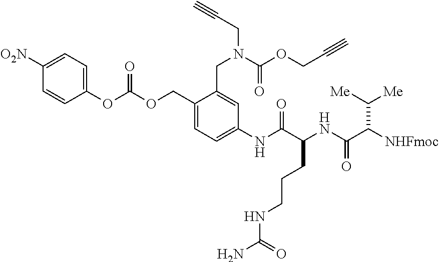

Following GENERAL PROCEDURE 1 with prop-2-yn-1-yl 5-((S)-2-((S)-2-((((9H-fluoren-9-yl)methoxy)carbonyl)amino)-3-methylbutanamido)-5-ureidopentanamido)-2-(hydroxymethyl)benzyl(prop-2-yn-1-yl)carbamate (556.0 mg, 0.740 mmol), prop-2-yn-1-yl 5-((S)-2-((S)-2-((((9H-fluoren-9-yl)methoxy)carbonyl)amino)-3-methylbutanamido)-5-ureidopentanamido)-2-((((4-nitrophenoxy)carbonyl)oxy)methyl)benzyl(prop-2-yn-1-yl)carbamate was obtained. LC/MS M+H=916.8, Rt=1.16 min (2 min acidic method).

### Synthesis of (S)-2-((S)-2-amino-3-methylbutanamido)-N-(4-(hydroxymethyl)phenyl)-5-ureidopentanamide

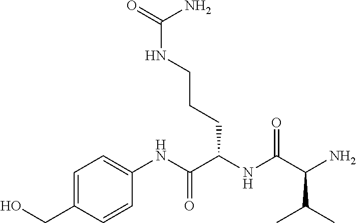 [SMILES: CC(C)[C@H](N)C(=O)N[C@@H](CCCNC(N)=O)C(=O)Nc1ccc(CO)cc1]

Following GENERAL PROCEDURE 4 described below with tert-butyl ((S)-1-(((S)-1-((4-(hydroxymethyl)phenyl)amino)-1-oxo-5-ureidopentan-2-yl)amino)-3-methyl-1-oxobutan-2-yl)carbamate (2.00 g, 4.17 mmol), (S)-2-((S)-2-amino-3-methylbutanamido)-N-(4-(hydroxymethyl)phenyl)-5-ureidopentanamide was obtained. LC/MS M+H=380.6, Rt=0.40 min (2 min acidic method).

### Synthesis of (S)-2-((S)-2-(3-(2-(2,5-dioxo-2,5-dihydro-1H-pyrrol-1-yl)ethoxy)propanamido)-3-methylbutanamido)-N-(4-(hydroxymethyl)phenyl)-5-ureidopentanamide

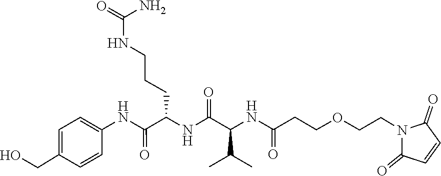 [SMILES: CC(C)[C@H](NC(=O)CCOCCN1C(=O)C=CC1=O)C(=O)N[C@@H](CCCNC(N)=O)C(=O)Nc1ccc(CO)cc1]

Following GENERAL PROCEDURE 5 described below with 2,5-dioxopyrrolidin-1-yl 3-(2-(2,5-dioxo-2,5-dihydro-1H-pyrrol-1-yl)ethoxy)propanoate (100.0 mg, 0.322 mmol) and(S)-2-((S)-2-amino-3-methylbutanamido)-N-(4-(hydroxymethyl)phenyl)-5-ureidopentanamide (175.0 mg, 0.355 mmol, 1.1 equiv.), (S)-2-((S)-2-(3-(2-(2,5-dioxo-2,5-dihydro-1H-pyrrol-1-yl)ethoxy)propanamido)-3-methylbutanamido)-N-(4-(hydroxymethyl)phenyl)-5-ureidopentanamide was obtained. LC/MS M+H=575.4, Rt=0.61 min (2 min basic method).

### Synthesis of 4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (4-nitrophenyl) carbonate

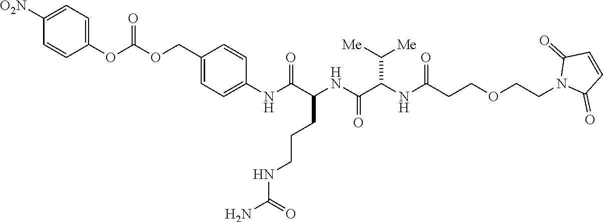 [SMILES: CC(C)[C@H](NC(=O)CCOCCN1C(=O)C=CC1=O)C(=O)N[C@@H](CCCNC(N)=O)C(=O)Nc1ccc(COC(=O)Oc2ccc([N+](=O)[O-])cc2)cc1]

Following GENERAL PROCEDURE 1 with(S)-2-((S)-2-(3-(2-(2,5-dioxo-2,5-dihydro-1H-pyrrol-1-yl)ethoxy)propanamido)-3-methylbutanamido)-N-(4-(hydroxymethyl)phenyl)-5-ureidopentanamide (126.0 mg, 0.219 mmol), 4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl (4-nitrophenyl) carbonate was obtained. LC/MS M+H=575.4, Rt=0.61 min (2 min basic method).

### Synthesis of tert-butyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate

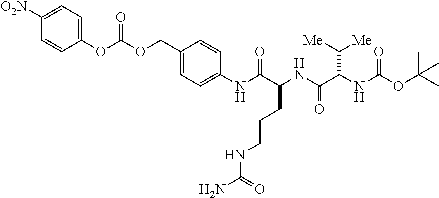 [SMILES: CC(C)[C@H](NC(=O)OC(C)(C)C)C(=O)N[C@@H](CCCNC(N)=O)C(=O)Nc1ccc(COC(=O)Oc2ccc([N+](=O)[O-])cc2)cc1]

Following GENERAL PROCEDURE 1 with tert-butyl ((S)-1-(((S)-1-((4-(hydroxymethyl)phenyl)amino)-1-oxo-5-ureidopentan-2-yl)amino)-3-methyl-1-oxobutan-2-yl)carbamate (200.0 mg, 0.417 mmol), tert-butyl ((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl)carbamate was obtained. LC/MS M+H=645.5, Rt=1.02 min (2 min acidic method).

### General Procedure 2

### Synthesis of 2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)-N-(4-((S)-2-((S)-2-((tert-butoxycarbonyl)amino)-3-methylbutanamido)-5-ureidopentanamido)-2-((methylamino)methyl)benzyl)-N,N-dimethylethan-1-aminium

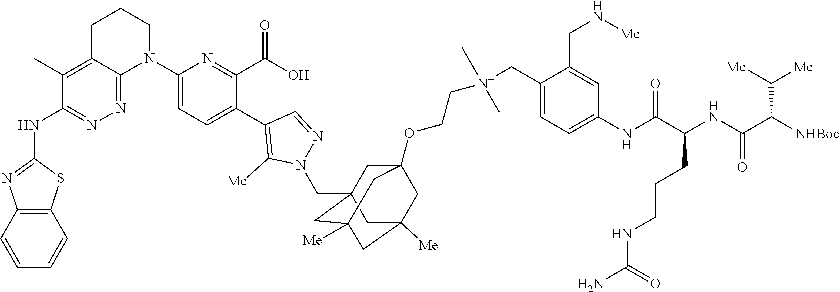 [SMILES: CNCc1cc(NC(=O)[C@H](CCCNC(N)=O)NC(=O)[C@@H](NC(=O)OC(C)(C)C)C(C)C)ccc1C[N+](C)(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2]

To a suspension of 6-(3-(benzo[d]thiazol-2-ylamino)-4-methyl-6,7-dihydropyrido[2,3-c]pyridazin-8 (5H)-yl)-3-(1-(((1r,3s,5R,7S)-3-(2-(dimethylamino)ethoxy)-5,7-dimethyladamantan-1-yl)methyl)-5-methyl-1H-pyrazol-4-yl)picolinic acid (25 mg, 0.033 mmol), (9H-fluoren-9-yl)methyl (5-((S)-2-((S)-2-((tert-butoxycarbonyl)amino)-3-methylbutanamido)-5-ureidopentanamido)-2-(chloromethyl)benzyl)(methyl)carbamate (25 mg, 0.033 mmol, 1.0 equiv.) and TBAl (12 mg, 0.033 mmol, 1.0 equiv.) in DMSO (1 mL) was added DIPEA (0.03 mL, 0.164 mmol, 5.0 equiv.) and stirred for 16 hours at RT. 2.0 M dimethylamine in THF (0.164 mL, 0.328 mmol, 10 equiv.) was added. After standing for 1.5 hours, the solution was purified by RP-HPLC ISCO gold chromatography (10-100% MeCN/H2O, 0.1% TFA modifier). Upon lyophilization, 2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)-N-(4-((S)-2-((S)-2-((tert-butoxycarbonyl)amino)-3-methylbutanamido)-5-ureidopentanamido)-2-((methylamino)methyl)benzyl)-N,N-dimethylethan-1-aminium was obtained. HRMS: M+=1266.3000; Rt=1.85 min (5 min acidic method).

### General Procedure 3

### Synthesis of 2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)-N-(4-((S)-2-((S)-2-((tert-butoxycarbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-N,N-dimethylethan-1-aminium

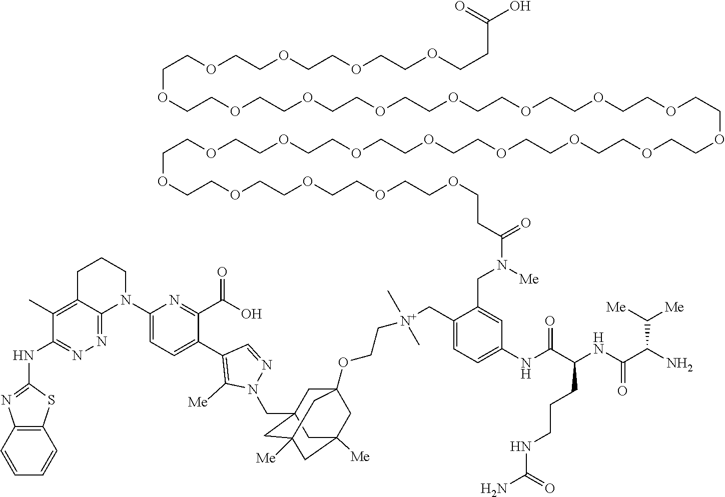 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](N)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

To a solution of 2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)-N-(4-((S)-2-((S)-2-((tert-butoxycarbonyl)amino)-3-methylbutanamido)-5-ureidopentanamido)-2-((methylamino)methyl)benzyl)-N,N-dimethylethan-1-aminium (42 mg, 0.027 mmol) and 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (42 mg, 0.032 mmol, 1.2 equiv.) in DMF (0.5 mL) was added DIPEA (0.023 mL, 0.133 mmol, 5.0 equiv.) and stirred for 5 hours at RT. DMSO (2 mL) was added and the solution was purified by RP-HPLC ISCO gold chromatography (10-100% MeCN/H2O, 0.1% TFA modifier). Upon lyophilization, 2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)-N-(4-((S)-2-((S)-2-((tert-butoxycarbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-N,N-dimethylethan-1-aminium was obtained. HRMS: M+=2465.7800; Rt=2.15 min (5 min acidic method).

### General Procedure 4

### Synthesis of N-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)-N,N-dimethylethan-1-aminium

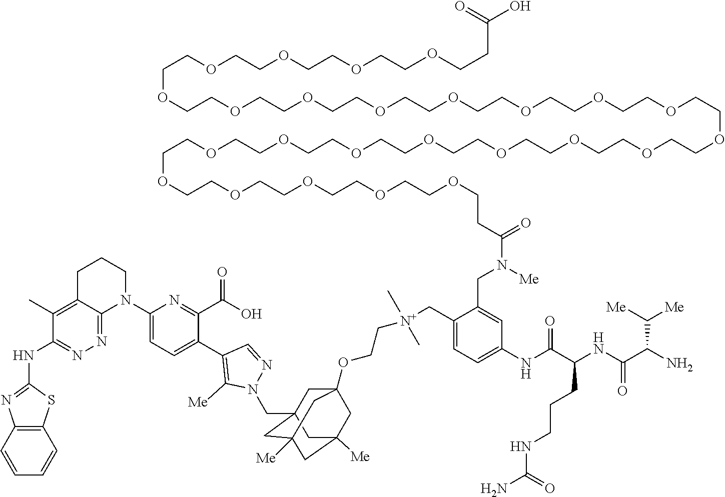 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](N)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

To a solution of 2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)-N-(4-((S)-2-((S)-2-((tert-butoxycarbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-N,N-dimethylethan-1-aminium (28 mg, 0.011 mmol) in CH₂Cl₂ (0.75 mL) at 0° C. in an ice bath was added trifluoroacetic acid (0.25 mL). The mixture was stirred for 1 hour in the ice bath, at which time the volatiles were removed in vacuo. DMSO (1.5 mL) was added and the solution was purified by RP-HPLC ISCO gold chromatography (10-100% MeCN/H2O, 0.1% TFA modifier). Upon lyophilization, N-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)-N,N-dimethylethan-1-aminium was obtained. HRMS: M+=2367.3101; Rt=1.86 min (5 min acidic method). For this general procedure, in some cases the amine was taken on as is without RP-HPLC purification.

### General Procedure 5

### Synthesis of 2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)-N-(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylethan-1-aminium (L11A-P27)

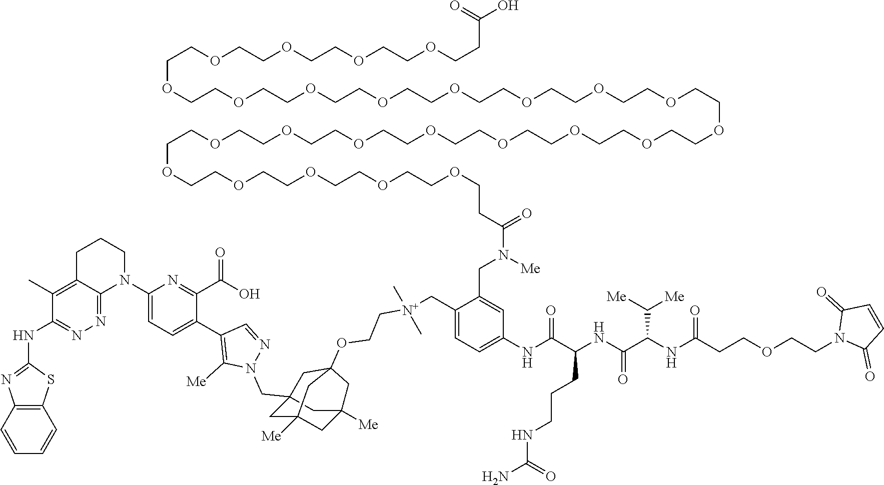 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

To a solution of N-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)-N,N-dimethylethan-1-aminium (10.0 mg, 0.004 mmol) and 2,5-dioxopyrrolidin-1-yl 3-(2-(2,5-dioxo-2,5-dihydro-1H-pyrrol-1-yl)ethoxy)propanoate (1.4 mg, 0.005 mmol, 1.2 equiv.) in DMF (0.5 mL) was added DIPEA (6.7 μL, 0.039 mmol, 10.0 equiv.). The mixture was stirred for 3.5 hours at RT. DMSO (1.5 mL) was added and the solution was purified by RP-HPLC ISCO gold chromatography (10-100% MeCN/H2O, 0.1% TFA modifier). Upon lyophilization, 2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)-N-(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylethan-1-aminium was obtained. HRMS: M+=2562.3401; Rt=2.04 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-((methylamino)methyl)benzyl)pyrrolidin-1-ium

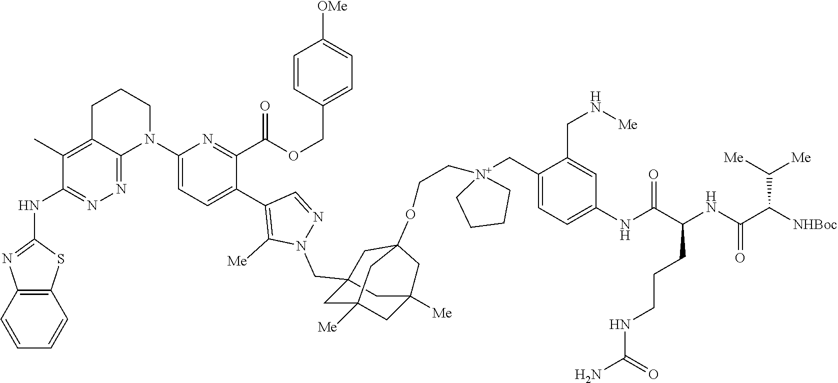 [SMILES: CNCc1cc(NC(=O)[C@H](CCCNC(N)=O)NC(=O)[C@@H](NC(=O)OC(C)(C)C)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)OCc6ccc(OC)cc6)c5C)(C4)C2)C3)CCCC1]

Following GENERAL PROCEDURE 2 with 4-methoxybenzyl 6-(3-(benzo[d]thiazol-2-ylamino)-4-methyl-6,7-dihydropyrido[2,3-c]pyridazin-8 (5H)-yl)-3-(1-(((1r,3R,5S,7s)-3,5-dimethyl-7-(2-(pyrrolidin-1-yl)ethoxy) adamantan-1-yl)methyl)-5-methyl-1H-pyrazol-4-yl) picolinate (30.0 mg, 0.033 mmol) and (9H-fluoren-9-yl)methyl (5-((S)-2-((S)-2-((tert-butoxycarbonyl)amino)-3-methylbutanamido)-5-ureidopentanamido)-2-(chloromethyl)benzyl)(methyl)carbamate (25.2 mg, 0.033 mmol, 1.0 equiv.), 1-(2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-((methylamino)methyl)benzyl)pyrrolidin-1-ium was obtained. HRMS: M+=1412.7600; Rt=2.22 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)pyrrolidin-1-ium

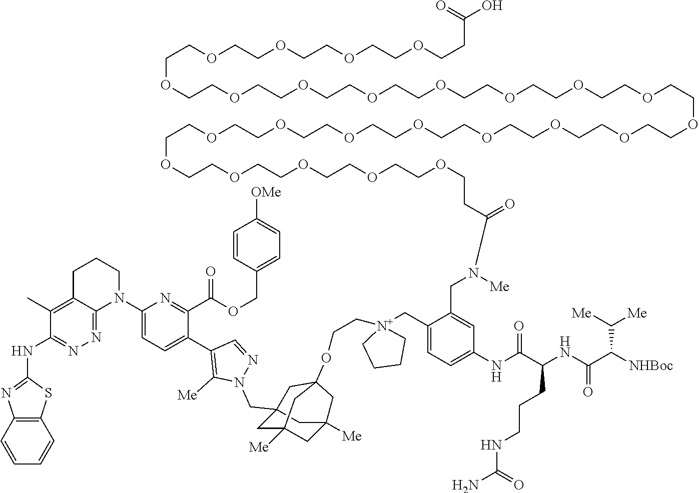 [SMILES: COc1ccc(COC(=O)c2nc(N3CCCc4c3nnc(Nc3nc5ccccc5s3)c4C)ccc2-c2cnn(CC34CC5(C)CC(C)(C3)CC(OCC[N+]3(Cc6ccc(NC(=O)[C@H](CCCNC(N)=O)NC(=O)[C@@H](NC(=O)OC(C)(C)C)C(C)C)cc6CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)CCCC3)(C5)C4)c2C)cc1]

Following GENERAL PROCEDURE 3 with 1-(2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-((methylamino)methyl)benzyl)pyrrolidin-1-ium (42.0 mg, 0.026 mmol) and 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (40.4 mg, 0.031 mmol, 1.2 equiv.), 1-(2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)pyrrolidin-1-ium was obtained. HRMS: M+=2613.4199; Rt=2.38 min (5 min acidic method).

### Synthesis of 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)ethyl)pyrrolidin-1-ium

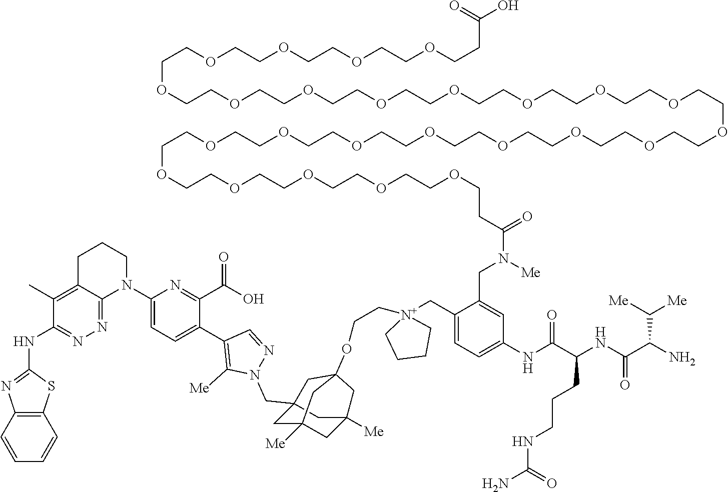 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](N)C(C)C)cc6CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)CCCC3)(C5)C4)c2C)c(C(=O)O)n1]

Following GENERAL PROCEDURE 4 with 1-(2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)pyrrolidin-1-ium (68.0 mg, 0.26 mmol), 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)ethyl)pyrrolidin-1-ium was obtained. HRMS: M+=2393.3301; Rt=1.85 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)ethyl)-1-(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)pyrrolidin-1-ium (L11A-P21)

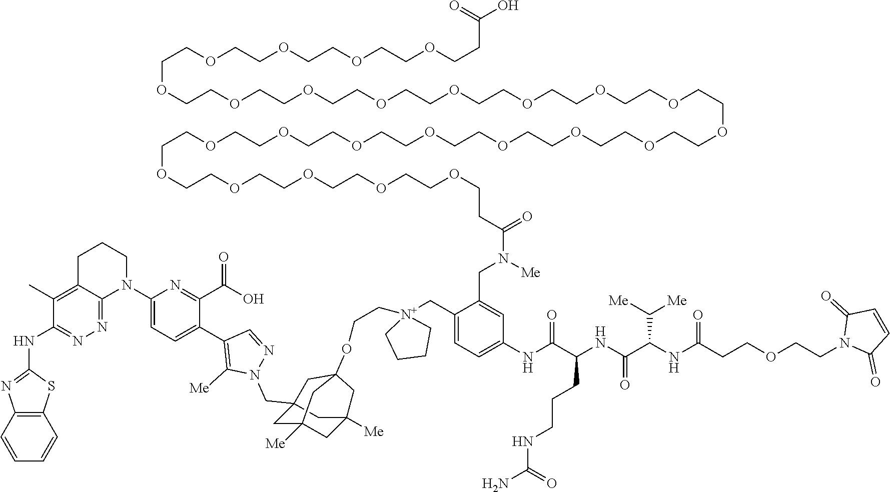 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)CCCC3)(C5)C4)c2C)c(C(=O)O)n1]

Following GENERAL PROCEDURE 5 with 1-(4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)ethyl)pyrrolidin-1-ium (26.1 mg, 0.011 mmol) and 2,5-dioxopyrrolidin-1-yl 3-(2-(2,5-dioxo-2,5-dihydro-1H-pyrrol-1-yl)ethoxy)propanoate (5.1 mg, 0.016 mmol, 1.5 equiv.), 1-(2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-carboxypyridin-3-yl)-5-methyl-1H-pyrazol-1-yl)methyl)-5,7-dimethyladamantan-1-yl)oxy)ethyl)-1-(2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)pyrrolidin-1-ium was obtained. HRMS: M+=2588.3899; Rt=2.05 min (5 min acidic method).

### General Procedure 6

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yloxy)methyl)benzyl)-N,N-dimethylprop-2-yn-1-aminium

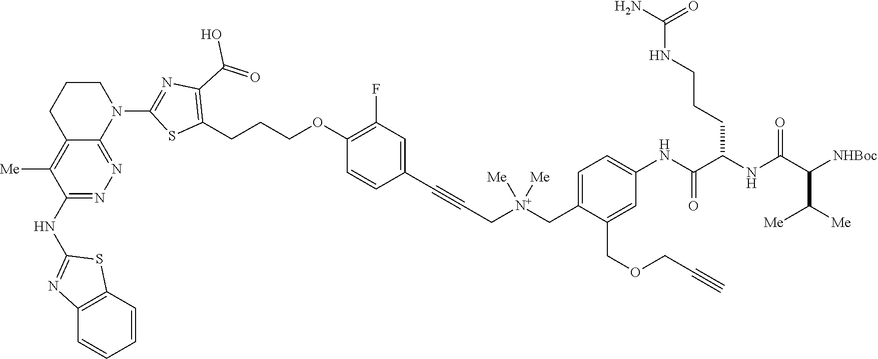 [SMILES: C#CCOCc1cc(NC(=O)[C@H](CCCNC(N)=O)NC(=O)[C@@H](NC(=O)OC(C)(C)C)C(C)C)ccc1C[N+](C)(C)CC#Cc1ccc(OCCCc2sc(N3CCCc4c3nnc(Nc3nc5ccccc5s3)c4C)nc2C(=O)O)c(F)c1]

To a suspension of 2-(3-(benzo[d]thiazol-2-ylamino)-4-methyl-6,7-dihydropyrido[2,3-c]pyridazin-8 (5H)-yl)-5-(3-(4-(3-(dimethylamino)prop-1-yn-1-yl)-2-fluorophenoxy)propyl)thiazole-4-carboxylic acid (75.0 mg, 0.114 mmol) and tert-butyl ((S)-1-(((S)-1-((4-(chloromethyl)-3-((prop-2-yn-1-yloxy)methyl)phenyl)amino)-1-oxo-5-ureidopentan-2-yl)amino)-3-methyl-1-oxobutan-2-yl)carbamate (103.0 mg, 0.182 mmol, 1.6 equiv.) in DMSO (2.0 ml) was added TBAl (67.4 mg, 0.182 mmol, 1.6 equiv.) and DIPEA (0.16 mL, 0.912 mmol, 9.0 equiv.). The mixture went into solution and was stirred for 2 hours at RT. After this time the solution was purified by RP-HPLC ISCO gold chromatography (10-70% MeCN/H2O, 0.1% TFA modifier). Upon lyophilization, 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yloxy)methyl)benzyl)-N,N-dimethylprop-2-yn-1-aminium was obtained. LCMS: M+=1187.6; Rt=0.93 min (2 min acidic method).

### General Procedure 7

### Synthesis of N-(2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium

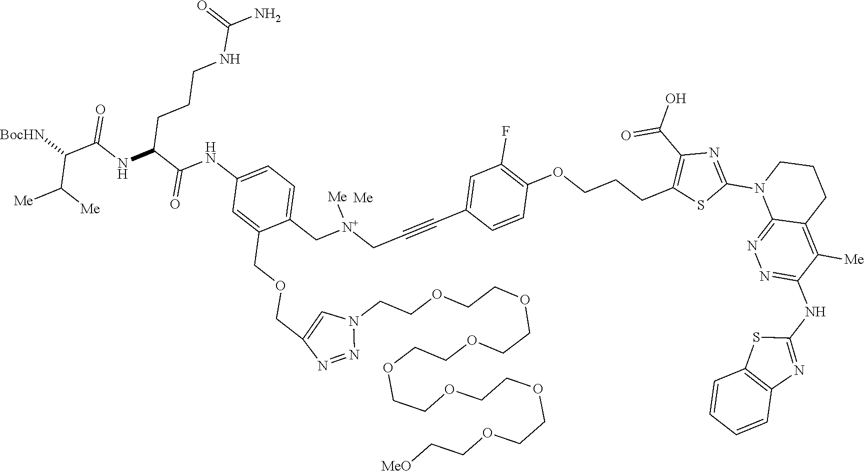

After a flask with 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yloxy)methyl)benzyl)-N,N-dimethylprop-2-yn-1-aminium (50.0 mg, 0.042 mmol), 25-azido-2,5,8,11,14,17,20,23-octaoxapentacosane (34.5 mg, 0.084 mmol, 2.0 equiv.), sodium (R)-2-((S)-1,2-dihydroxyethyl)-4-hydroxy-5-oxo-2,5-dihydrofuran-3-olate (12.5 mg, 0.63 mmol, 1.5 equiv.) and copper (II) sulfate pentahydrate (2.1 mg, 0.008 mmol, 0.2 equiv.) was sealed and evacuated/purge with N₂ 3×, tert.-butanol (5.0 mL) and water (0.5 mL) were added via syringe. The mixture was stirred for 2 hours at RT. DMSO (1 mL) was added and the solution was purified by RP-HPLC ISCO gold chromatography (0-100% MeCN/H2O, 0.1% TFA modifier). Upon lyophilization, N-(2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium was obtained. HRMS: M+=1596.7531; Rt=1.18 min (2 min acidic method). For this general procedure, in some cases instead of tert.-butanol, DMF or DMSO was used.

### Synthesis of N-(2-(((1-(2,5,8,11,14,17,20,23-octaoxapentacosan-25-yl)-1H-1,2,3-triazol-4-yl)methoxy)methyl)-4-((S)-2-((S)-2-amino-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium

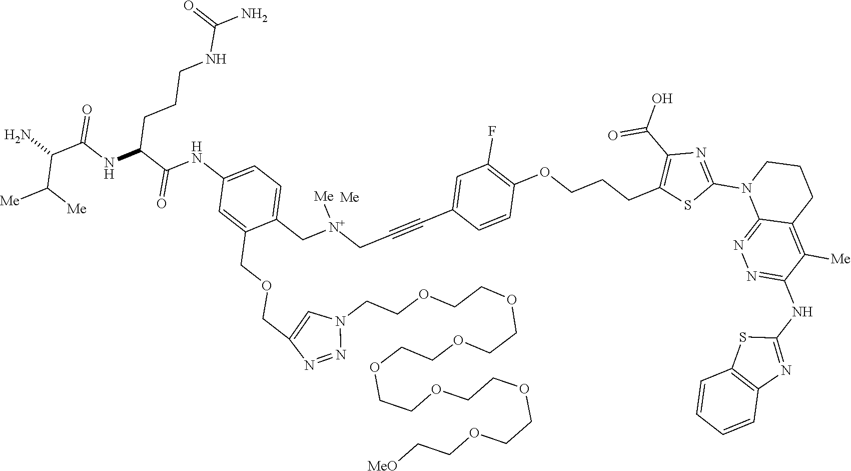

Following GENERAL PROCEDURE 4 with N-(2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium (30.0 mg, 0.019 mmol), N-(2-(((1-(2,5,8,11,14,17,20,23-octaoxapentacosan-25-yl)-1H-1,2,3-triazol-4-yl)methoxy)methyl)-4-((S)-2-((S)-2-amino-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium was obtained. LCMS: M+=1497.2; Rt=1.94 min (5 min acidic method).

### Synthesis of N-(2-(((1-(2,5,8,11,14,17,20,23-octaoxapentacosan-25-yl)-1H-1,2,3-triazol-4-yl)methoxy)methyl)-4-((R)-2-((R)-2-(3-(2-(2,5-dioxo-2,5-dihydro-1H-pyrrol-1-yl)ethoxy)propanamido)-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium (L8A-P1)

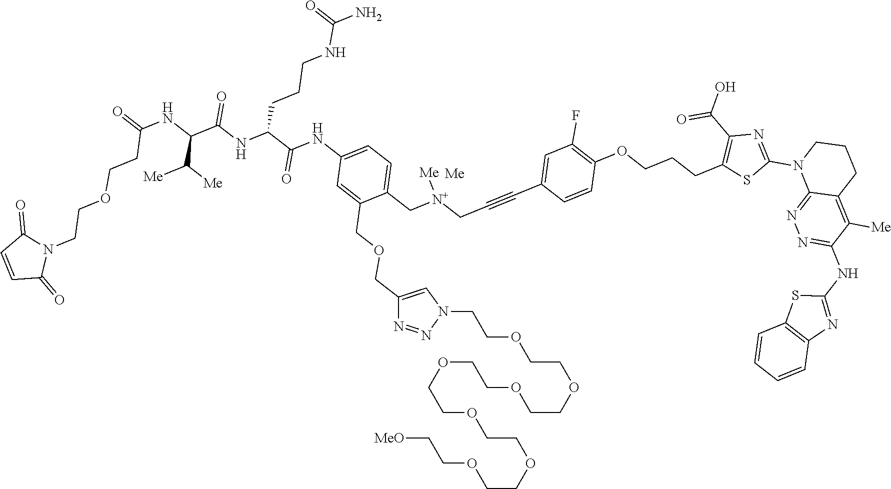 [SMILES: COCCOCCOCCOCCOCCOCCOCCOCCn1cc(COCc2cc(NC(=O)[C@@H](CCCNC(N)=O)NC(=O)[C@H](NC(=O)CCOCCN3C(=O)C=CC3=O)C(C)C)ccc2C[NH+](C)CC#Cc2ccc(OCCCc3sc(N4CCCc5c4nnc(Nc4nc6ccccc6s4)c5C)nc3C(=O)O)c(F)c2)nn1 ; "Me"]

Following GENERAL PROCEDURE 5 with N-(2-(((1-(2,5,8,11,14,17,20,23-octaoxapentacosan-25-yl)-1H-1,2,3-triazol-4-yl)methoxy)methyl)-4-((S)-2-((S)-2-amino-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium (24.0 mg, 0.016 mmol), N-(2-(((1-(2,5,8,11,14,17,20,23-octaoxapentacosan-25-yl)-1H-1,2,3-triazol-4-yl)methoxy)methyl)-4-((R)-2-((R)-2-(3-(2-(2,5-dioxo-2,5-dihydro-1H-pyrrol-1-yl)ethoxy)propanamido)-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium was obtained. HRMS: M+=1691.7500; Rt=4.35 min (5 min acidic method).

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-(((1-(26-carboxy-3,6,9,12,15,18,21,24-octaoxahexacosyl)-1H-1,2,3-triazol-4-yl)methoxy)methyl)benzyl)-N,N-dimethylprop-2-yn-1-aminium

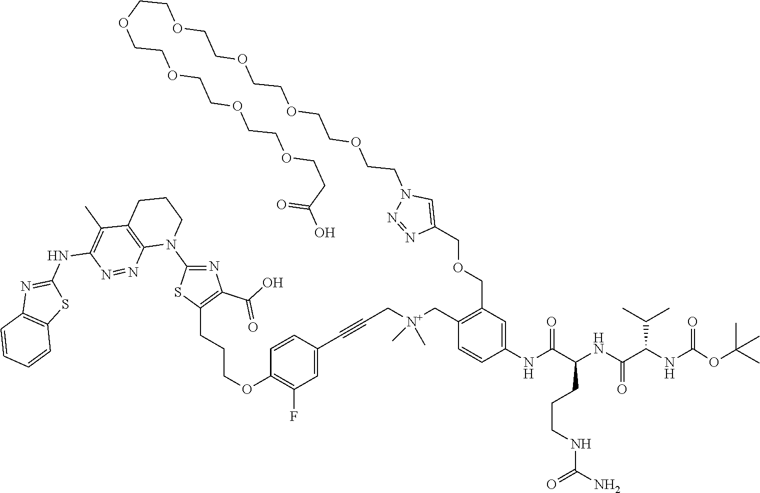 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)OC(C)(C)C)C(C)C)cc3COCc3cn(CCOCCOCCOCCOCCOCCOCCOCCOCCC(=O)O)nn3)cc2F)s1]

Following GENERAL PROCEDURE 7 with 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yloxy)methyl)benzyl)-N,N-dimethylprop-2-yn-1-aminium (50.0 mg, 0.042 mmol) and 1-azido-3,6,9,12,15,18,21,24-octaoxaheptacosan-27-oic acid (39.4 mg, 0.084 mmol, 2.0 equiv.), 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-(((1-(26-carboxy-3,6,9,12,15,18,21,24-octaoxahexacosyl)-1H-1,2,3-triazol-4-yl)methoxy)methyl)benzyl)-N,N-dimethylprop-2-yn-1-aminium was obtained. LCMS: ½M+=828.1; Rt=0.71 min (2 min acidic method).

### GENERAL PROCEDURE 8

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(2-(((1-(26-carboxy-3,6,9,12,15,18,21,24-octaoxahexacosyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium (L7A-P1)

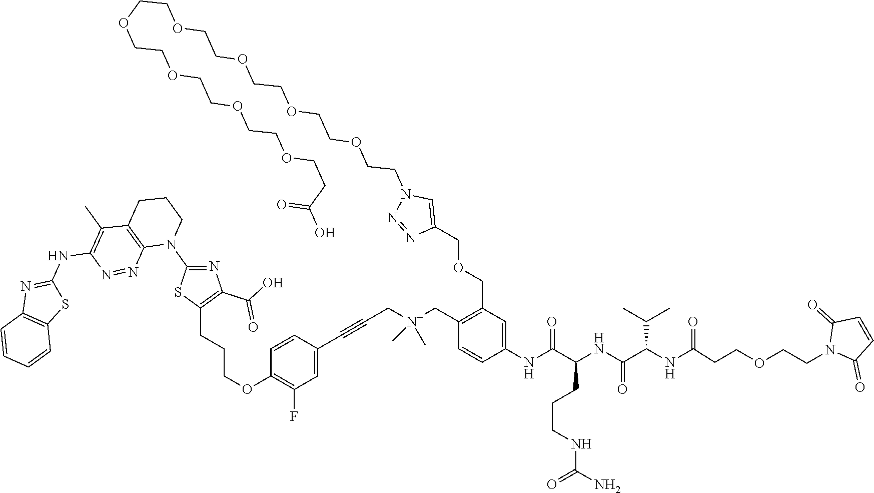 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3COCc3cn(CCOCCOCCOCCOCCOCCOCCOCCOCCC(=O)O)nn3)cc2F)s1]

A solution of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-(((1-(26-carboxy-3,6,9,12,15,18,21,24-octaoxahexacosyl)-1H-1,2,3-triazol-4-yl)methoxy)methyl)benzyl)-N,N-dimethylprop-2-yn-1-aminium (32.2 mg, 0.019 mmol) in DCM/TFA (3:1, 2.6 mL) was cooled to 0° C. and stirred for 1 hour at this temperature. After the mixture was evaporated under reduced pressure to yield crude de-Boc intermediate, crude was solved in DMF (0.5 mL) and followed by adding 2,5-dioxopyrrolidin-1-yl 3-(2-(2,5-dioxo-2,5-dihydro-1H-pyrrol-1-yl)ethoxy)propanoate (12.1 mg, 0.039 mmol, 2.0 equiv.) and DIPEA (0.1 mL, 0.584 mmol, 30.0 equiv.). Mixture was stirred for 30 min. at RT. The solution was purified by RP-HPLC ISCO gold chromatography (0-100% MeCN/H2O, 0.1% TFA modifier). Upon lyophilization, 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(2-(((1-(26-carboxy-3,6,9,12,15,18,21,24-octaoxahexacosyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium was obtained. HRMS: M+=1749.7400; Rt=2.51 min (5 min acidic method).

### Synthesis of N-(4-((S)-2-((S)-2-amino-3-methylbutanamido)-5-ureidopentanamido)-2-((prop-2-yn-1-yloxy)methyl)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium

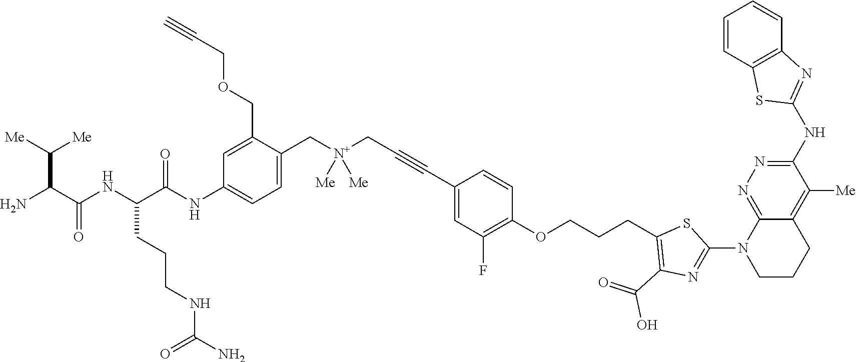 [SMILES: C#CCOCc1cc(NC(=O)[C@H](CCCNC(N)=O)NC(=O)[C@@H](N)C(C)C)ccc1C[N+](C)(C)CC#Cc1ccc(OCCCc2sc(N3CCCc4c3nnc(Nc3nc5ccccc5s3)c4C)nc2C(=O)O)c(F)c1]

Following GENERAL PROCEDURE 4 with 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yloxy)methyl)benzyl)-N,N-dimethylprop-2-yn-1-aminium (263.0 mg, 0.221 mmol), N-(4-((S)-2-((S)-2-amino-3-methylbutanamido)-5-ureidopentanamido)-2-((prop-2-yn-1-yloxy)methyl)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium was obtained. HRMS: M+=1087.2700; Rt=1.85 min (5 min acidic method).

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yloxy)methyl)benzyl)-N,N-dimethylprop-2-yn-1-aminium

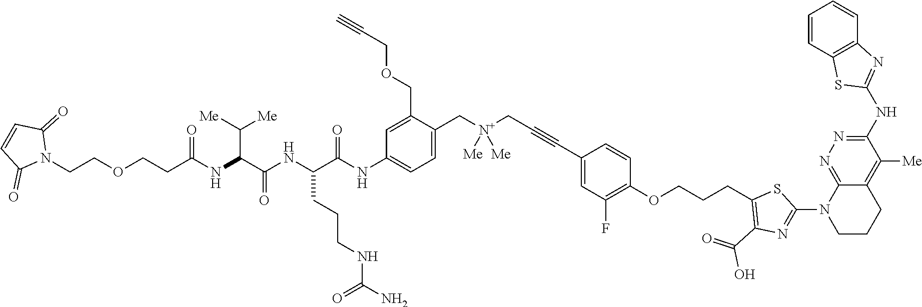

Following GENERAL PROCEDURE 5 with N-(4-((S)-2-((S)-2-amino-3-methylbutanamido)-5-ureidopentanamido)-2-((prop-2-yn-1-yloxy)methyl)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium (77.0 mg, 0.050 mmol) and 2,5-dioxopyrrolidin-1-yl 3-(2-(2,5-dioxo-2,5-dihydro-1H-pyrrol-1-yl)ethoxy)propanoate (23.2 mg, 0.075 mmol, 1.5 equiv.), 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yloxy)methyl)benzyl)-N,N-dimethylprop-2-yn-1-aminium was obtained. HRMS: M+=1282.4800; Rt=2.15 min (5 min acidic method).

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(2-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium (L109A-P1)

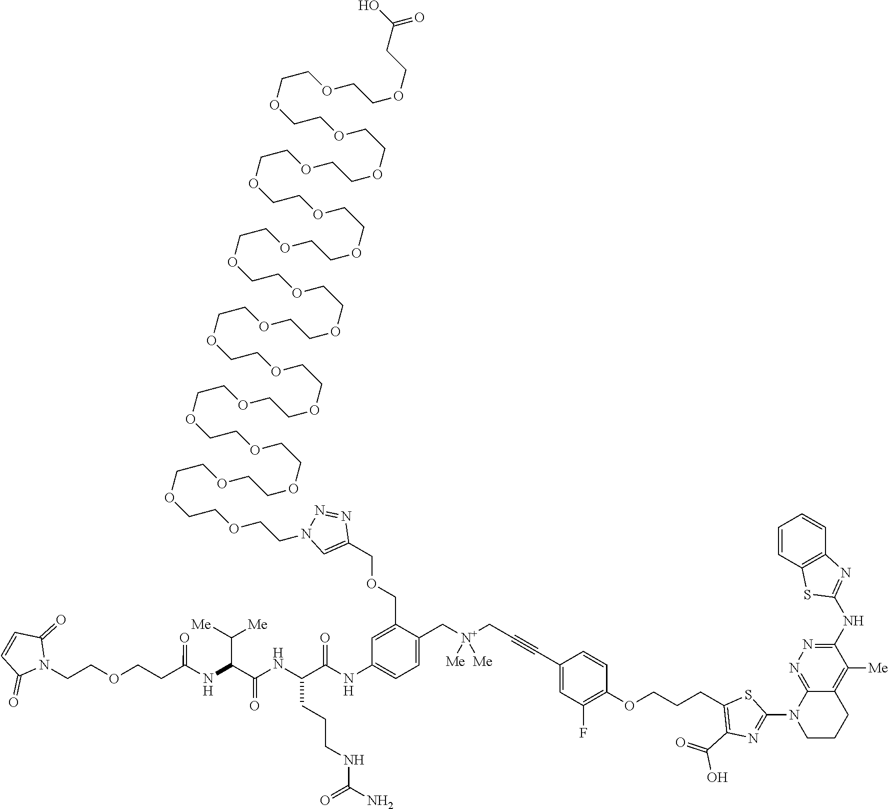

Following GENERAL PROCEDURE 7 with 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yloxy)methyl)benzyl)-N,N-dimethylprop-2-yn-1-aminium (51.8 mg, 0.037 mmol) and 1-azido-3,6,9,12,15,18,21,24,27,30,33,36,39,42,45,48,51,54,57,60,63,66,69,72-tetracosaoxapentaheptacontan-75-oic acid (87.0 mg, 0.074 mmol, 2.0 equiv.), 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(2-(((1-(74-carboxy-3,6,9,12,15,18,21,24,27,30,33,36,39,42,45,48,51,54,57,60,63,66,69,72-tetracosaoxatetraheptacontyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium was obtained. HRMS: M+=2453.8899; Rt=2.17 min (5 min acidic method).

### Synthesis of 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yloxy)methyl)benzyl)-N,N-dimethylprop-2-yn-1-aminium

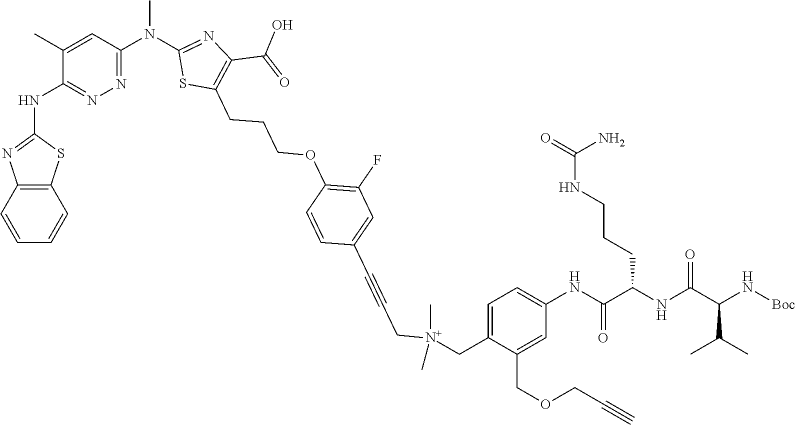 [SMILES: C#CCOCc1cc(NC(=O)[C@H](CCCNC(N)=O)NC(=O)[C@@H](NC(=O)OC(C)(C)C)C(C)C)ccc1C[N+](C)(C)CC#Cc1ccc(OCCCc2sc(N(C)c3cc(C)c(Nc4nc5ccccc5s4)nn3)nc2C(=O)O)c(F)c1]

Following GENERAL PROCEDURE 6 with 2-((6-(benzo[d]thiazol-2-ylamino)-5-methylpyridazin-3-yl)(methyl)amino)-5-(3-(4-(3-(dimethylamino)prop-1-yn-1-yl)-2-fluorophenoxy)propyl)thiazole-4-carboxylic acid (50.0 mg, 0.079 mmol) and tert-butyl ((S)-1-(((S)-1-((4-(chloromethyl)-3-((prop-2-yn-1-yloxy)methyl)phenyl)amino)-1-oxo-5-ureidopentan-2-yl)amino)-3-methyl-1-oxobutan-2-yl)carbamate (71.7 mg, 0.127 mmol, 1.6 equiv.), 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yloxy)methyl)benzyl)-N,N-dimethylprop-2-yn-1-aminium was obtained. LCMS: M+=1162.2; Rt=0.94 min (2 min basic method).

### Synthesis of 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-(((1-(26-carboxy-3,6,9,12,15,18,21,24-octaoxahexacosyl)-1H-1,2,3-triazol-4-yl)methoxy)methyl)benzyl)-N,N-dimethylprop-2-yn-1-aminium

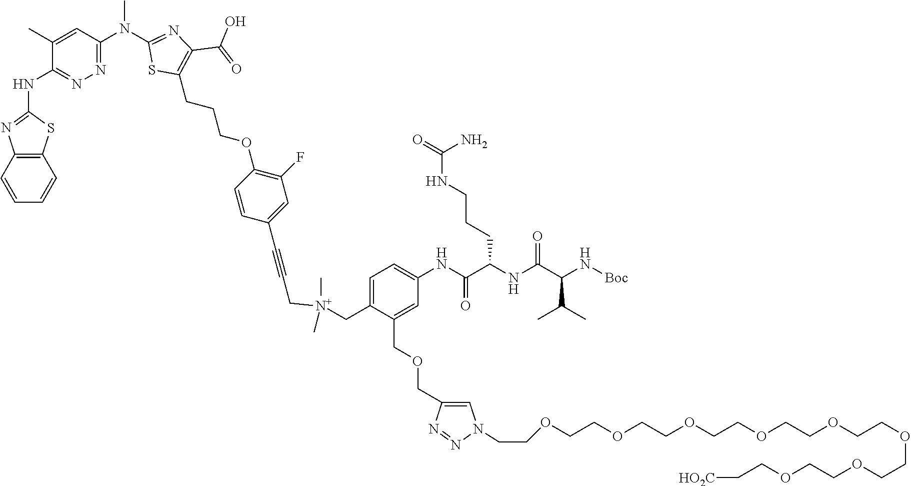 [SMILES: Cc1cc(N(C)c2nc(C(=O)O)c(CCCOc3ccc(C#CC[N+](C)(C)Cc4ccc(NC(=O)[C@H](CCCNC(N)=O)NC(=O)[C@@H](NC(=O)OC(C)(C)C)C(C)C)cc4COCc4cn(CCOCCOCCOCCOCCOCCOCCOCCOCCC(=O)O)nn4)cc3F)s2)nnc1Nc1nc2ccccc2s1]

Following GENERAL PROCEDURE 7 with 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yloxy)methyl)benzyl)-N,N-dimethylprop-2-yn-1-aminium (40.0 mg, 0.034 mmol) and 1-azido-3,6,9,12,15,18,21,24-octaoxaheptacosan-27-oic acid (25.8 mg, 0.055 mmol, 1.6 equiv.), 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-(((1-(26-carboxy-3,6,9,12,15,18,21,24-octaoxahexacosyl)-1H-1,2,3-triazol-4-yl)methoxy)methyl)benzyl)-N,N-dimethylprop-2-yn-1-aminium was obtained. LCMS: M/2+=815.4; Rt=0.99 min (2 min acidic method).

### Synthesis of 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(2-(((1-(26-carboxy-3,6,9,12,15,18,21,24-octaoxahexacosyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium (L7A-P2)

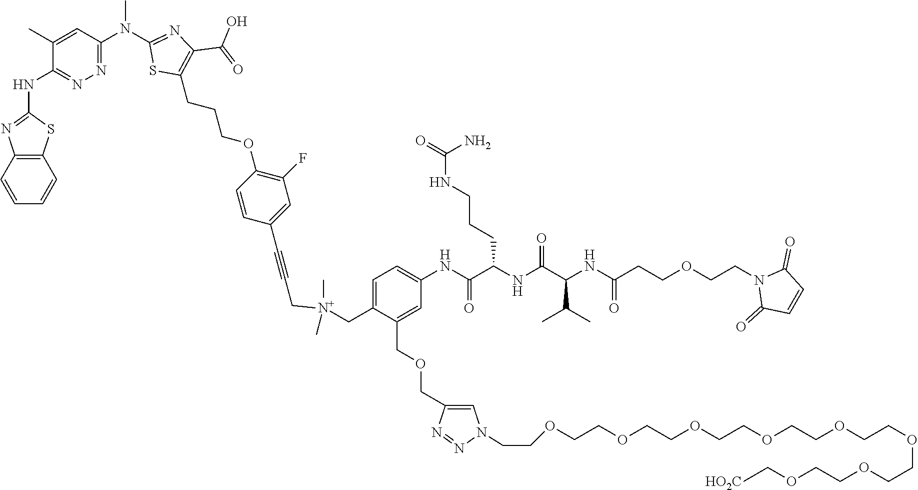 [SMILES: Cc1cc(N(C)c2nc(C(=O)O)c(CCCOc3ccc(C#CC[N+](C)(C)Cc4ccc(NC(=O)[C@H](CCCNC(N)=O)NC(=O)[C@@H](NC(=O)CCOCCN5C(=O)C=CC5=O)C(C)C)cc4COCc4cn(CCOCCOCCOCCOCCOCCOCCOCCOCCC(=O)O)nn4)cc3F)s2)nnc1Nc1nc2ccccc2s1]

Following GENERAL PROCEDURE 8 with 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-(((1-(26-carboxy-3,6,9,12,15,18,21,24-octaoxahexacosyl)-1H-1,2,3-triazol-4-yl)methoxy)methyl)benzyl)-N,N-dimethylprop-2-yn-1-aminium (37.0 mg, 0.023 mmol) and 2,5-dioxopyrrolidin-1-yl 3-(2-(2,5-dioxo-2,5-dihydro-1H-pyrrol-1-yl)ethoxy)propanoate (10.6 mg, 0.034 mmol, 1.5 equiv.), 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(2-(((1-(26-carboxy-3,6,9,12,15,18,21,24-octaoxahexacosyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium was obtained. LCMS: M-=1722.9; Rt=0.91 min (2 min acidic method).

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((methyl((prop-2-yn-1-yloxy)carbonyl)amino)methyl)benzyl)-N,N-dimethylprop-2-yn-1-aminium

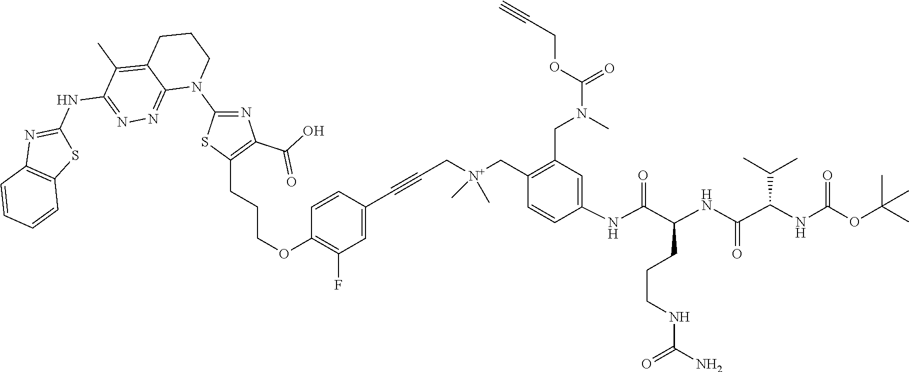 [SMILES: C#CCOC(=O)N(C)Cc1cc(NC(=O)[C@H](CCCNC(N)=O)NC(=O)[C@@H](NC(=O)OC(C)(C)C)C(C)C)ccc1C[N+](C)(C)CC#Cc1ccc(OCCCc2sc(N3CCCc4c3nnc(Nc3nc5ccccc5s3)c4C)nc2C(=O)O)c(F)c1]

Following GENERAL PROCEDURE 6 with 2-(3-(benzo[d]thiazol-2-ylamino)-4-methyl-6,7-dihydropyrido[2,3-c]pyridazin-8 (5H)-yl)-5-(3-(4-(3-(dimethylamino)prop-1-yn-1-yl)-2-fluorophenoxy)propyl)thiazole-4-carboxylic acid (118.0 mg, 0.170 mmol) and prop-2-yn-1-yl 5-((S)-2-((S)-2-((tert-butoxycarbonyl)amino)-3-methylbutanamido)-5-ureidopentanamido)-2-(chloromethyl)benzyl(methyl)carbamate (127.0 mg, 0.204 mmol, 1.2 equiv.), 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((methyl((prop-2-yn-1-yloxy)carbonyl)amino)methyl)benzyl)-N,N-dimethylprop-2-yn-1-aminium was obtained. HRMS: M+=1244.5100; Rt=2.42 min (5 min acidic method).

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((methyl((prop-2-yn-1-yloxy)carbonyl)amino)methyl)benzyl)-N,N-dimethylprop-2-yn-1-aminium

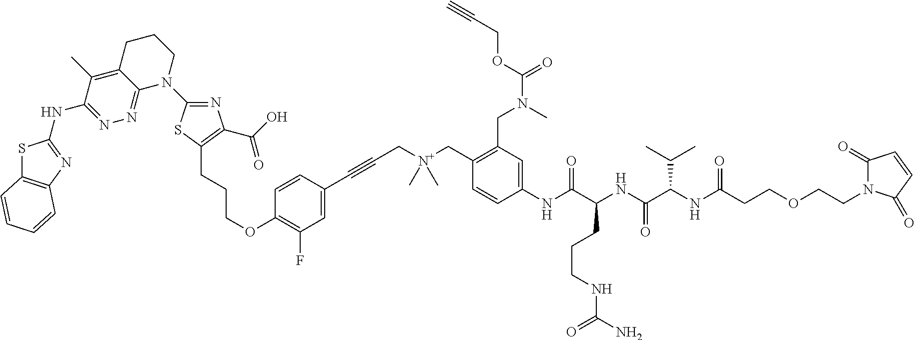 [SMILES: C#CCOC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1]

Following GENERAL PROCEDURE 8 with 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((methyl((prop-2-yn-1-yloxy)carbonyl)amino)methyl)benzyl)-N,N-dimethylprop-2-yn-1-aminium (65.0 mg, 0.052 mmol) and 2,5-dioxopyrrolidin-1-yl 3-(2-(2,5-dioxo-2,5-dihydro-1H-pyrrol-1-yl)ethoxy)propanoate (32.4 mg, 0.104 mmol, 2.0 equiv.), 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((methyl((prop-2-yn-1-yloxy)carbonyl)amino)methyl)benzyl)-N,N-dimethylprop-2-yn-1-aminium was obtained. LCMS: M+=1341.1; Rt-2.20 min (5 min acidic method).

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(2-(((((1-(26-carboxy-3,6,9,12,15,18,21,24-octaoxahexacosyl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium (L3A-P1)

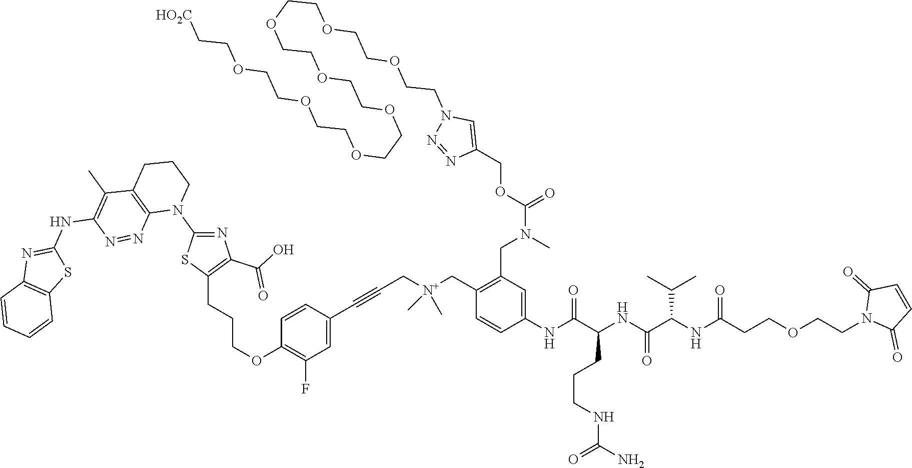 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)OCc3cn(CCOCCOCCOCCOCCOCCOCCOCCOCCC(=O)O)nn3)cc2F)s1]

Following GENERAL PROCEDURE 7 with 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((methyl((prop-2-yn-1-yloxy)carbonyl)amino)methyl)benzyl)-N,N-dimethylprop-2-yn-1-aminium (65.0 mg, 0.049 mmol) and 1-azido-3,6,9,12,15,18,21,24-octaoxaheptacosan-27-oic acid (45.4 mg, 0.097 mmol, 2.0 equiv.), 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(2-(((((1-(26-carboxy-3,6,9,12,15,18,21,24-octaoxahexacosyl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium was obtained. HRMS: M+=1806.7700; Rt=2.05 min (5 min acidic method).

### Synthesis of N-(2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-((tert-butoxycarbonyl)amino)-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium

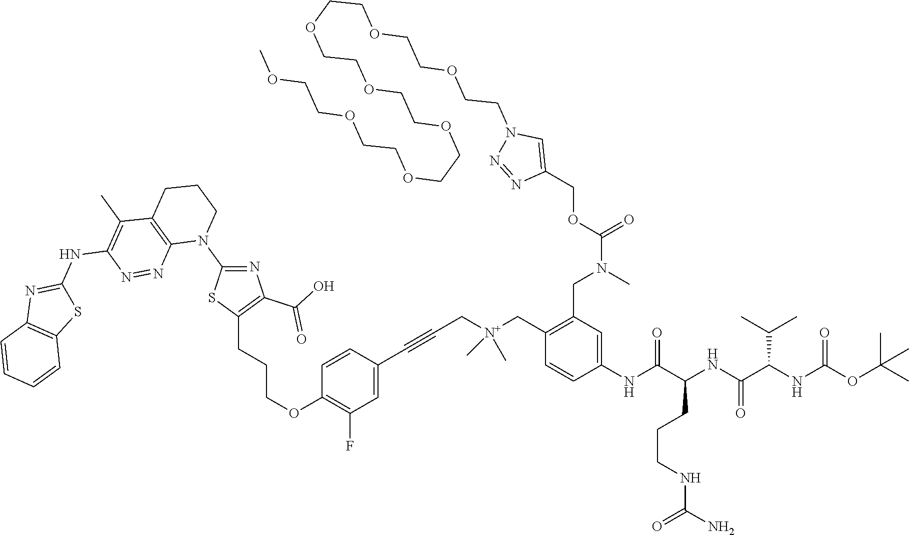 [SMILES: COCCOCCOCCOCCOCCOCCOCCOCCn1cc(COC(=O)N(C)Cc2cc(NC(=O)[C@H](CCCNC(N)=O)NC(=O)[C@@H](NC(=O)OC(C)(C)C)C(C)C)ccc2C[N+](C)(C)CC#Cc2ccc(OCCCc3sc(N4CCCc5c4nnc(Nc4nc6ccccc6s4)c5C)nc3C(=O)O)c(F)c2)nn1]

Following GENERAL PROCEDURE 7 with 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((methyl((prop-2-yn-1-yloxy)carbonyl)amino)methyl)benzyl)-N,N-dimethylprop-2-yn-1-aminium (36.0 mg, 0.029 mmol) and 25-azido-2,5,8,11,14,17,20,23-octaoxapentacosane (23.7 mg, 0.058 mmol, 2.0 equiv.), N-(2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-((tert-butoxycarbonyl)amino)-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium was obtained. HRMS: M+=1653.7500; Rt=2.29 min (5 min acidic method).

### Synthesis of N-(2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium (L4A-P1)

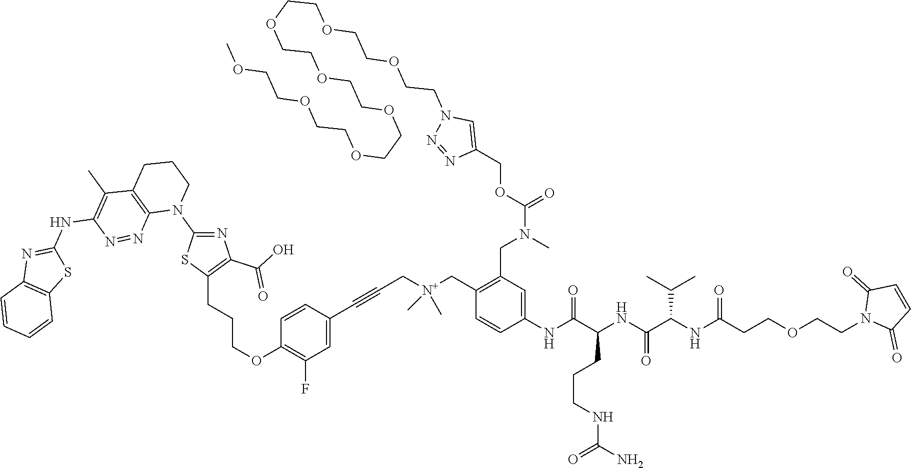 [SMILES: COCCOCCOCCOCCOCCOCCOCCOCCn1cc(COC(=O)N(C)Cc2cc(NC(=O)[C@H](CCCNC(N)=O)NC(=O)[C@@H](NC(=O)CCOCCN3C(=O)C=CC3=O)C(C)C)ccc2C[N+](C)(C)CC#Cc2ccc(OCCCc3sc(N4CCCc5c4nnc(Nc4nc6ccccc6s4)c5C)nc3C(=O)O)c(F)c2)nn1]

Following GENERAL PROCEDURE 8 with N-(2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-((tert-butoxycarbonyl)amino)-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium (19.6 mg, 0.012 mmol) and 2,5-dioxopyrrolidin-1-yl 3-(2-(2,5-dioxo-2,5-dihydro-1H-pyrrol-1-yl)ethoxy)propanoate (7.4 mg, 0.024 mmol, 2.0 equiv.), N-(2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium was obtained. HRMS: M+=1748.7600; Rt=2.15 min (5 min acidic method).

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl)-N,N-dimethylprop-2-yn-1-aminium

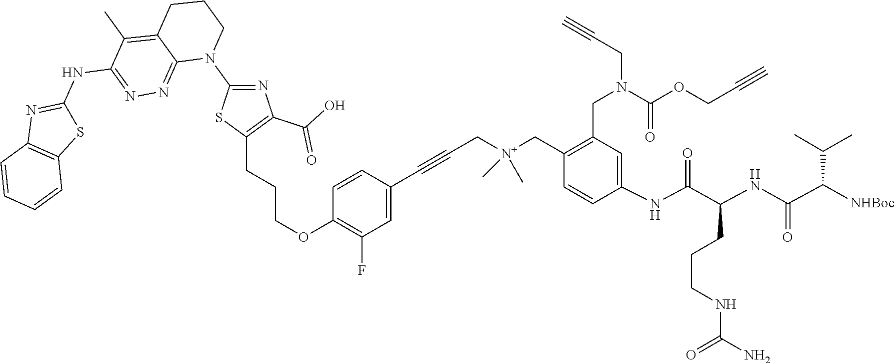 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1C[N+](C)(C)CC#Cc1ccc(OCCCc2sc(N3CCCc4c3nnc(Nc3nc5ccccc5s3)c4C)nc2C(=O)O)c(F)c1]

Following GENERAL PROCEDURE 6 with 2-(3-(benzo[d]thiazol-2-ylamino)-4-methyl-6,7-dihydropyrido[2,3-c]pyridazin-8 (5H)-yl)-5-(3-(4-(3-(dimethylamino)prop-1-yn-1-yl)-2-fluorophenoxy)propyl)thiazole-4-carboxylic acid (50.0 mg, 0.076 mmol) and prop-2-yn-1-yl 5-((S)-2-((S)-2-((tert-butoxycarbonyl)amino)-3-methylbutanamido)-5-ureidopentanamido)-2-(chloromethyl)benzyl(prop-2-yn-1-yl)carbamate (73.8 mg, 0.114 mmol, 1.5 equiv.), 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl)-N,N-dimethylprop-2-yn-1-aminium was obtained. LCMS: M+=1269.2; Rt=2.24 min (5 min basic method).

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yl((prop-2-yn-1-yloxy)carbonyl)amino)methyl)benzyl)-N,N-dimethylprop-2-yn-1-aminium

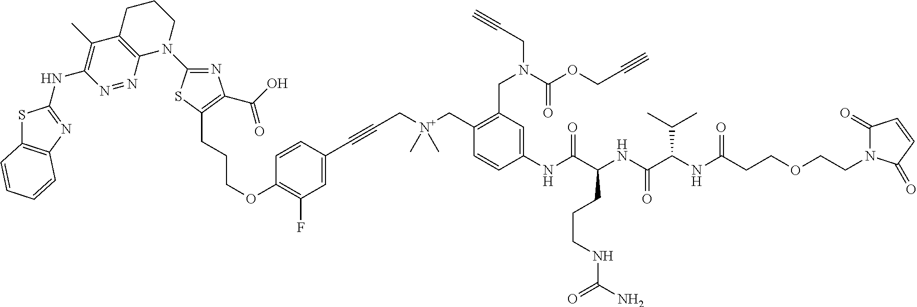 [SMILES: C#CCOC(=O)N(CC#C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1]

Following GENERAL PROCEDURE 8 with 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl)-N,N-dimethylprop-2-yn-1-aminium (45.9 mg, 0.036 mmol) and 2,5-dioxo-2,5-dihydro-1H-pyrrol-1-yl 3-(2-(2,5-dioxo-2,5-dihydro-1H-pyrrol-1-yl)ethoxy)propanoate (22.3 mg, 0.072 mmol, 2.0 equiv.), 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yl((prop-2-yn-1-yloxy)carbonyl)amino)methyl)benzyl)-N,N-dimethylprop-2-yn-1-aminium was obtained. HRMS: M+=1363.5100; Rt=2.26 min (5 min acidic method).

### Synthesis of N-(2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium (L1A-P1)

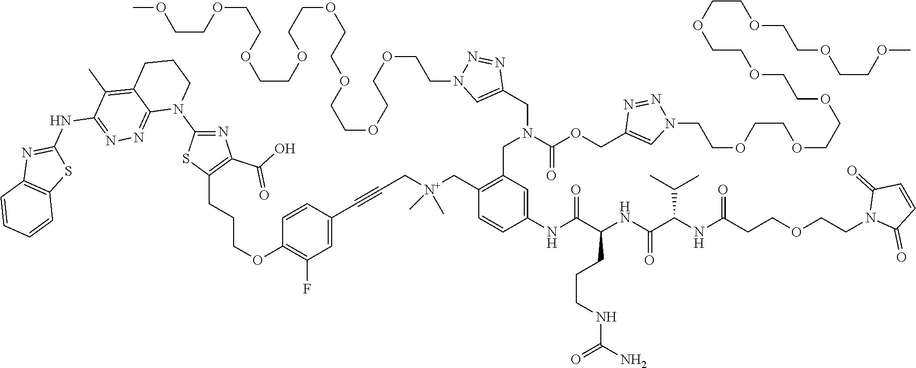 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](NC(=O)CCOCCN3C(=O)C=CC3=O)C(C)C)ccc2C[N+](C)(C)CC#Cc2ccc(OCCCc3sc(N4CCCc5c4nnc(Nc4nc6ccccc6s4)c5C)nc3C(=O)O)c(F)c2)nn1]

Following GENERAL PROCEDURE 7 with 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yl((prop-2-yn-1-yloxy)carbonyl)amino)methyl)benzyl)-N,N-dimethylprop-2-yn-1-aminium (21.9 mg, 0.016 mmol) and 1-azido-3,6,9,12,15,18,21,24-octaoxahexacosane (51.0 mg, 0.120 mmol, 7.5 equiv.), N-(2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N,N-dimethylprop-2-yn-1-aminium was obtained. HRMS: M+=2181.9800; Rt=2.31 min (5 min acidic method).

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(2-(((((1-(26-carboxy-3,6,9,12,15,18,21,24-octaoxahexacosyl)-1H-1,2,3-triazol-4-yl)methoxy)carbonyl)((1-(26-carboxy-3,6,9,12,15,18,21,24-octaoxahexacosyl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium (L10A-P1)

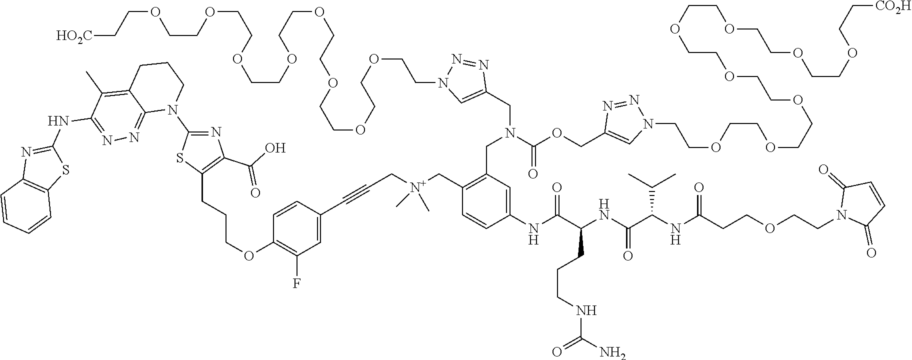 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(Cc3cn(CCOCCOCCOCCOCCOCCOCCOCCOCCC(=O)O)nn3)C(=O)OCc3cn(CCOCCOCCOCCOCCOCCOCCOCCOCCC(=O)O)nn3)cc2F)s1]

Following GENERAL PROCEDURE 7 with 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yl((prop-2-yn-1-yloxy)carbonyl)amino)methyl)benzyl)-N,N-dimethylprop-2-yn-1-aminium (20.0 mg, 0.015 mmol) and 1-azido-3,6,9,12,15,18,21,24-octaoxaheptacosan-27-oic acid (51.4 mg, 0.110 mmol, 7.5 equiv.), 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(2-(((((1-(26-carboxy-3,6,9,12,15,18,21,24-octaoxahexacosyl)-1H-1,2,3-triazol-4-yl)methoxy)carbonyl)((1-(26-carboxy-3,6,9,12,15,18,21,24-octaoxahexacosyl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium was obtained. HRMS: M+=2298.0100; Rt-2.44 min (5 min acidic method).

### Synthesis of 2-((6-(benzo[d]thiazol-2-ylamino)-5-methylpyridazin-3-yl)(methyl)amino)-5-(3-(4-(3-((4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl)dimethylammonio)prop-1-yn-1-yl)-2-fluorophenoxy)propyl)thiazole-4-carboxylate

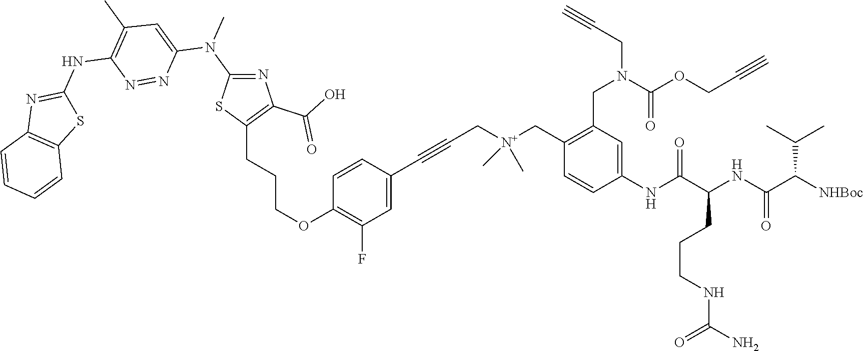 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1C[N+](C)(C)CC#Cc1ccc(OCCCc2sc(N(C)c3cc(C)c(Nc4nc5ccccc5s4)nn3)nc2C(=O)O)c(F)c1]

Following GENERAL PROCEDURE 6 with 2-((6-(benzo[d]thiazol-2-ylamino)-5-methylpyridazin-3-yl)(methyl)amino)-5-(3-(4-(3-(dimethylamino)prop-1-yn-1-yl)-2-fluorophenoxy)propyl)thiazole-4-carboxylic acid (50.0 mg, 0.079 mmol) and prop-2-yn-1-yl 5-((S)-2-((S)-2-((tert-butoxycarbonyl)amino)-3-methylbutanamido)-5-ureidopentanamido)-2-(chloromethyl)benzyl(prop-2-yn-1-yl)carbamate (61.5 mg, 0.095 mmol, 1.2 equiv.), 2-((6-(benzo[d]thiazol-2-ylamino)-5-methylpyridazin-3-yl)(methyl)amino)-5-(3-(4-(3-((4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl)dimethylammonio)prop-1-yn-1-yl)-2-fluorophenoxy)propyl)thiazole-4-carboxylate was obtained. LCMS: M+=1243.2; Rt=2.27 min (5 min acidic method).

### Synthesis of 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)((1-(26-carboxy-3,6,9,12,15,18,21,24-octaoxahexacosyl)-1H-1,2,3-triazol-4-yl)methyl)amino)methyl)benzyl)-N,N-dimethylprop-2-yn-1-aminium

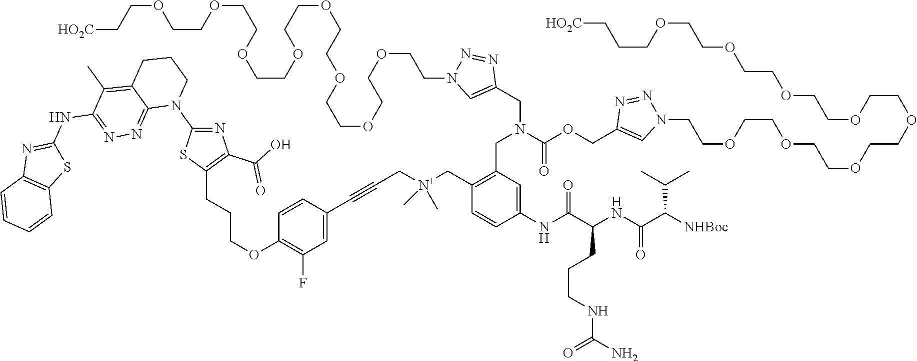 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)OC(C)(C)C)C(C)C)cc3CN(Cc3cn(CCOCCOCCOCCOCCOCCOCCOCCOCCC(=O)O)nn3)C(=O)OCc3cn(CCOCCOCCOCCOCCOCCOCCOCCOCCCC(=O)O)nn3)cc2F)s1]

Following GENERAL PROCEDURE 7 with 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl)-N,N-dimethylprop-2-yn-1-aminium (21.8 mg, 0.018 mmol) and 1-azido-3,6,9,12,15,18,21,24-octaoxaheptacosan-27-oic acid (32.8 mg, 0.070 mmol, 4.0 equiv.), 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)((1-(26-carboxy-3,6,9,12,15,18,21,24-octaoxahexacosyl)-1H-1,2,3-triazol-4-yl)methyl)amino)methyl)benzyl)-N,N-dimethylprop-2-yn-1-aminium was obtained. HRMS: M+=2176.8301; Rt=2.25 min (5 min acidic method).

### Synthesis of 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(2-(((((1-(26-carboxy-3,6,9,12,15,18,21,24-octaoxahexacosyl)-1H-1,2,3-triazol-4-yl)methoxy)carbonyl)((1-(26-carboxy-3,6,9,12,15,18,21,24-octaoxahexacosyl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium (L10A-P2)

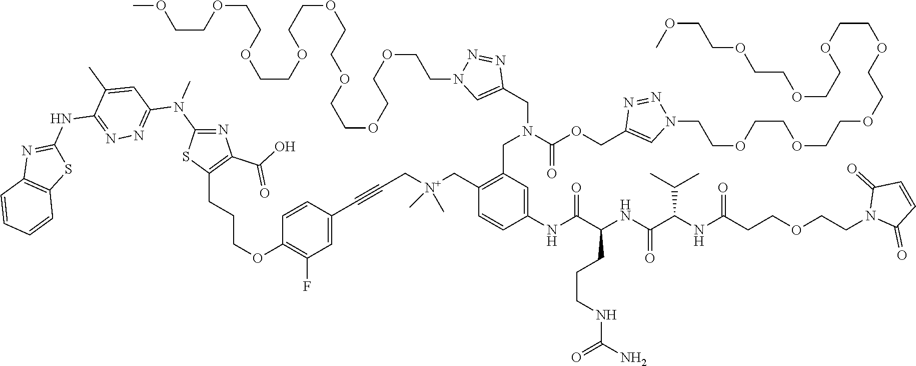 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](NC(=O)CCOCCN3C(=O)C=CC3=O)C(C)C)ccc2C[N+](C)(C)CC#Cc2ccc(OCCCc3sc(N(C)c4cc(C)c(Nc5nc6ccccc6s5)nn4)nc3C(=O)O)c(F)c2)nn1]

Following GENERAL PROCEDURE 8 with 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)((1-(26-carboxy-3,6,9,12,15,18,21,24-octaoxahexacosyl)-1H-1,2,3-triazol-4-yl)methyl)amino)methyl)benzyl)-N,N-dimethylprop-2-yn-1-aminium (17.8 mg, 0.008 mmol) and 2,5-dioxopyrrolidin-1-yl 3-(2-(2,5-dioxo-2,5-dihydro-1H-pyrrol-1-yl)ethoxy)propanoate (10.2 mg, 0.033 mmol, 4.0 equiv.), 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(2-(((((1-(26-carboxy-3,6,9,12,15,18,21,24-octaoxahexacosyl)-1H-1,2,3-triazol-4-yl)methoxy)carbonyl)((1-(26-carboxy-3,6,9,12,15,18,21,24-octaoxahexacosyl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium was obtained. HRMS: M+=2271.8186; Rt=2.12 min (5 min acidic method).

### Synthesis of N-(2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-((tert-butoxycarbonyl)amino)-3-methylbutanamido)-5-ureidopentanamido)benzyl)-3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N,N-dimethylprop-2-yn-1-aminium

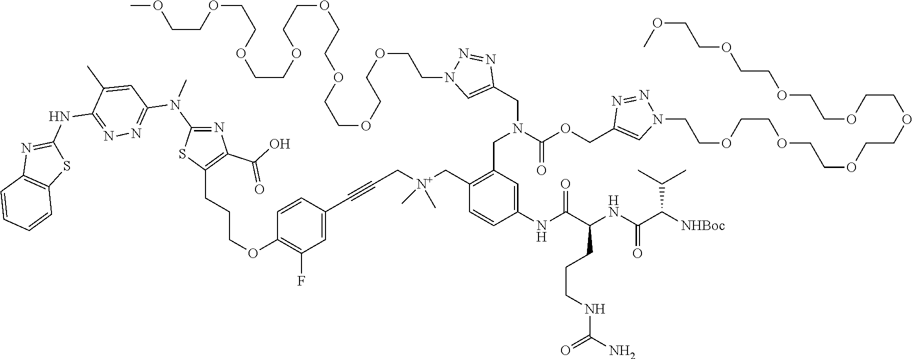 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](NC(=O)OC(C)(C)C)C(C)C)ccc2C[N+](C)(C)CC#Cc2ccc(OCCCc3sc(N(C)c4cc(C)c(Nc5nc6ccccc6s5)nn4)nc3C(=O)O)c(F)c2)nn1]

Following GENERAL PROCEDURE 7 with 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl)-N,N-dimethylprop-2-yn-1-aminium (22.6 mg, 0.018 mmol) and 25-azido-2,5,8,11,14,17,20,23-octaoxapentacosane (29.8 mg, 0.073 mmol, 4.0 equiv.), N-(2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-((tert-butoxycarbonyl)amino)-3-methylbutanamido)-5-ureidopentanamido)benzyl)-3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N,N-dimethylprop-2-yn-1-aminium was obtained. LCMS: M/2+=1032.3; Rt=2.25 min (5 min acidic method).

### Synthesis of N-(2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N,N-dimethylprop-2-yn-1-aminium (L1A-P2)

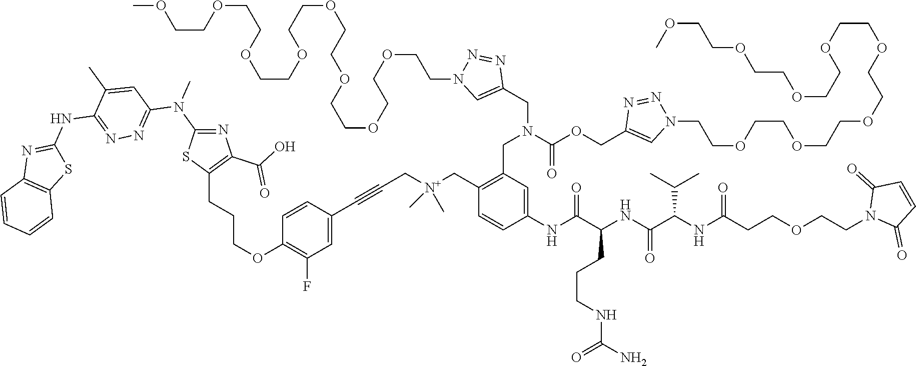 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](NC(=O)CCOCCN3C(=O)C=CC3=O)C(C)C)ccc2C[N+](C)(C)CC#Cc2ccc(OCCCc3sc(N(C)c4cc(C)c(Nc5nc6ccccc6s5)nn4)nc3C(=O)O)c(F)c2)nn1]

Following GENERAL PROCEDURE 8 with N-(2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-((tert-butoxycarbonyl)amino)-3-methylbutanamido)-5-ureidopentanamido)benzyl)-3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N,N-dimethylprop-2-yn-1-aminium (23.0 mg, 0.011 mmol) and 2,5-dioxopyrrolidin-1-yl 3-(2-(2,5-dioxo-2,5-dihydro-1H-pyrrol-1-yl)ethoxy)propanoate (10.4 mg, 0.033 mmol, 3.0 equiv.), N-(2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N,N-dimethylprop-2-yn-1-aminium was obtained. HRMS: M+=2155.8176; Rt-2.23 min (5 min acidic method).

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)benzyl)-N,N-dimethylprop-2-yn-1-aminium

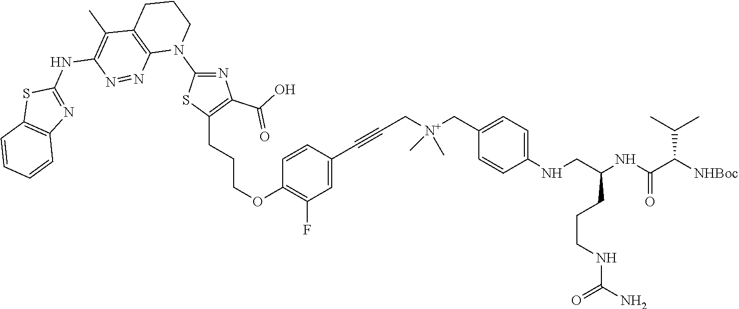 [SMILES: Cc1c(Nc2nc3ccccc3s2)nnc2c1CCCN2c1nc(C(=O)O)c(CCCOc2ccc(C#CC[N+](C)(C)Cc3ccc(NC[C@H](CCCNC(N)=O)NC(=O)[C@@H](NC(=O)OC(C)(C)C)C(C)C)cc3)cc2F)s1]

Following GENERAL PROCEDURE 6 with 2-(3-(benzo[d]thiazol-2-ylamino)-4-methyl-6,7-dihydropyrido[2,3-c]pyridazin-8 (5H)-yl)-5-(3-(4-(3-(dimethylamino)prop-1-yn-1-yl)-2-fluorophenoxy)propyl)thiazole-4-carboxylic acid (21.5 mg, 0.033 mmol) and tert-butyl ((S)-1-(((S)-1-((4-(chloromethyl)phenyl)amino)-1-oxo-5-ureidopentan-2-yl)amino)-3-methyl-1-oxobutan-2-yl)carbamate (21.2 mg, 0.042 mmol, 1.3 equiv.), 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)benzyl)-N,N-dimethylprop-2-yn-1-aminium was obtained. LCMS: M+=1119.3; Rt=2.15 min (5 min acidic method).

### Synthesis of 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium (L9A-P1)

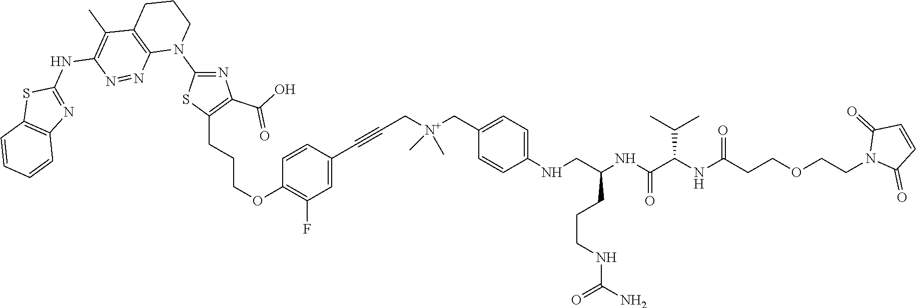 [SMILES: Cc1c(Nc2nc3ccccc3s2)nnc2c1CCCN2c1nc(C(=O)O)c(CCCOc2ccc(C#CC[N+](C)(C)Cc3ccc(NC[C@H](CCCNC(N)=O)NC(=O)[C@@H](NC(=O)CCOCCN4C(=O)C=CC4=O)C(C)C)cc3)cc2F)s1]

Following GENERAL PROCEDURE 8 with 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)benzyl)-N,N-dimethylprop-2-yn-1-aminium (36.6 mg, 0.033 mmol) and 2,5-dioxopyrrolidin-1-yl 3-(2-(2,5-dioxo-2,5-dihydro-1H-pyrrol-1-yl)ethoxy)propanoate (20.3 mg, 0.065 mmol, 2.0 equiv.), 3-(4-(3-(2-(3-(benzo[d]thiazol-2-ylamino)-4-methyl-6,7-dihydropyrido[2,3-c]pyridazin-8 (5H)-yl)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium was obtained. HRMS: M+=1214.4700; Rt=2.10 min (5 min acidic method).

### Synthesis of 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)benzyl)-N,N-dimethylprop-2-yn-1-aminium

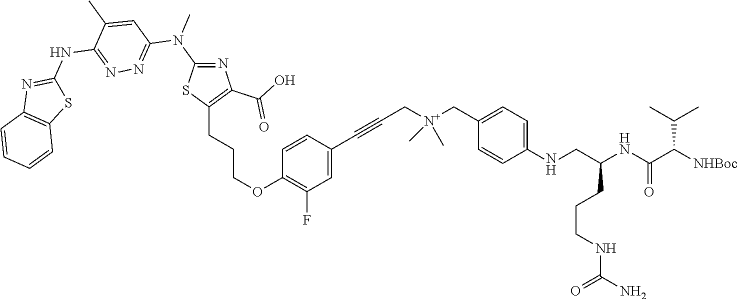 [SMILES: Cc1cc(N(C)c2nc(C(=O)O)c(CCCOc3ccc(C#CC[N+](C)(C)Cc4ccc(NC[C@H](CCCNC(N)=O)NC(=O)[C@@H](NC(=O)OC(C)(C)C)C(C)C)cc4)cc3F)s2)nnc1Nc1nc2ccccc2s1]

Following GENERAL PROCEDURE 6 with 2-((6-(benzo[d]thiazol-2-ylamino)-5-methylpyridazin-3-yl)(methyl)amino)-5-(3-(4-(3-(dimethylamino)prop-1-yn-1-yl)-2-fluorophenoxy)propyl)thiazole-4-carboxylic acid (25.0 mg, 0.040 mmol) and tert-butyl ((S)-1-(((S)-1-((4-(chloromethyl)phenyl)amino)-1-oxo-5-ureidopentan-2-yl)amino)-3-methyl-1-oxobutan-2-yl)carbamate (25.6 mg, 0.051 mmol, 1.3 equiv.), 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)benzyl)-N,N-dimethylprop-2-yn-1-aminium was obtained. LCMS: M+=1094.1; Rt=2.14 min (5 min acidic method).

### Synthesis of 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium (L9A-P2)

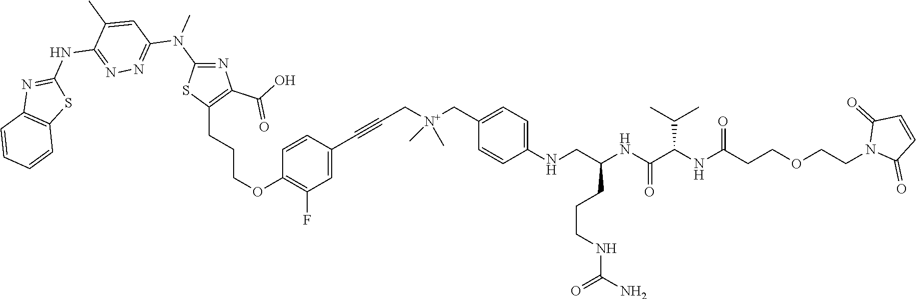 [SMILES: Cc1cc(N(C)c2nc(C(=O)O)c(CCCOc3ccc(C#CC[N+](C)(C)Cc4ccc(NC[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1]

Following GENERAL PROCEDURE 8 with 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-((tert-butoxycarbonyl)amino)-3-methylbutanamido)-5-ureidopentanamido)benzyl)-N,N-dimethylprop-2-yn-1-aminium (31.6 mg, 0.029 mmol) and 2,5-dioxopyrrolidin-1-yl 3-(2-(2,5-dioxo-2,5-dihydro-1H-pyrrol-1-yl)ethoxy)propanoate (26.9 mg, 0.087 mmol, 3.0 equiv.), 3-(4-(3-(2-((6-(benzo[d]thiazol-2-ylamino)-5-methylpyridazin-3-yl)(methyl)amino)-4-carboxythiazol-5-yl)propoxy)-3-fluorophenyl)-N-(4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-N,N-dimethylprop-2-yn-1-aminium was obtained. HRMS: M+=1188.4500; Rt=2.07 min (5 min acidic method).

### General Procedure 9

### Synthesis of 4-methoxybenzyl 6-(3-(benzo[d]thiazol-2-ylamino)-4-methyl-6,7-dihydropyrido[2,3-c]pyridazin-8 (5H)-yl)-3-(1-(((1r,3s,5R,7S)-3-(2-((((4-((S)-2-((S)-2-((tert- butoxycarbonyl)amino)-3-methylbutanamido)-5-ureidopentanamido)-2-((methylamino)methyl)benzyl)oxy)carbonyl)(3-hydroxypropyl)amino)ethoxy)-5,7-dimethyladamantan-1-yl)methyl)-5-methyl-1H-pyrazol-4-yl) picolinate

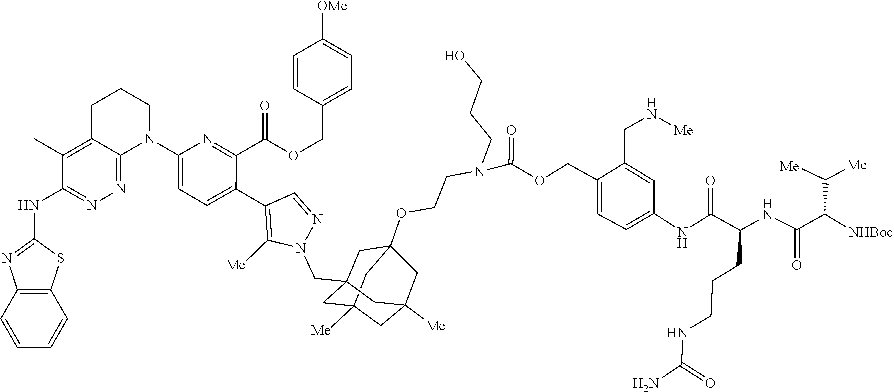 [SMILES: CNCc1cc(NC(=O)[C@H](CCCNC(N)=O)NC(=O)[C@@H](NC(=O)OC(C)(C)C)C(C)C)ccc1COC(=O)N(CCCO)CCOC12CC3(C)CC(C)(CC(Cn4ncc(-c5ccc(N6CCCc7c6nnc(Nc6nc8ccccc8s6)c7C)nc5C(=O)OCc5ccc(OC)cc5)c4C)(C3)C1)C2]

To a solution of 4-methoxybenzyl 6-(3-(benzo[d]thiazol-2-ylamino)-4-methyl-6,7-dihydropyrido[2,3-c]pyridazin-8 (5H)-yl)-3-(1-(((1r,3s,5R,7S)-3-(2-((3-hydroxypropyl)amino)ethoxy)-5,7-dimethyladamantan-1-yl)methyl)-5-methyl-1H-pyrazol-4-yl) picolinate (30.0 mg, 0.033 mmol) and (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate (35.9 mg, 0.039 mmol, 1.2 equiv.) in DMF (1.0 mL) was added DIPEA (0.03 mL, 0.164 mmol, 5.0 equiv.) and the mixture was stirred for 16 hours at RT. After the carbamate formation, 2M dimethylamine in THF (0.164 mL, 0.329 mmol, 1.0 equiv.) was added and stirred mixture for 1.5 hours. DMSO (2.0 mL) was added and the solution was purified by RP-HPLC ISCO gold chromatography (10-100% MeCN/H2O, 0.1% TFA modifier). Upon lyophilization, 4-methoxybenzyl 6-(3-(benzo[d]thiazol-2-ylamino)-4-methyl-6,7-dihydropyrido[2,3-c]pyridazin-8 (5H)-yl)-3-(1-(((1r,3s,5R,7S)-3-(2-((((4-((S)-2-((S)-2-((tert-butoxycarbonyl)amino)-3-methylbutanamido)-5-ureidopentanamido)-2-((methylamino)methyl)benzyl)oxy)carbonyl)(3-hydroxypropyl)amino)ethoxy)-5,7-dimethyladamantan-1-yl)methyl)-5-methyl-1H-pyrazol-4-yl) picolinate was obtained. HRMS: M+=1460.7500; Rt=2.31 min (5 min acidic method).

### Synthesis of 1-(2-((((2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)(3-hydroxypropyl) carbamoyl)oxy)methyl)-5-((S)-2-((S)-2-((tert-butoxycarbonyl)amino)-3-methylbutanamido)-5-ureidopentanamido)phenyl)-2-methyl-3-oxo-6,9,12,15,18,21,24,27,30,33,36,39,42,45,48,51,54,57,60,63,66,69,72,75,78-pentacosaoxa-2-azahenoctacontan-81-oic acid

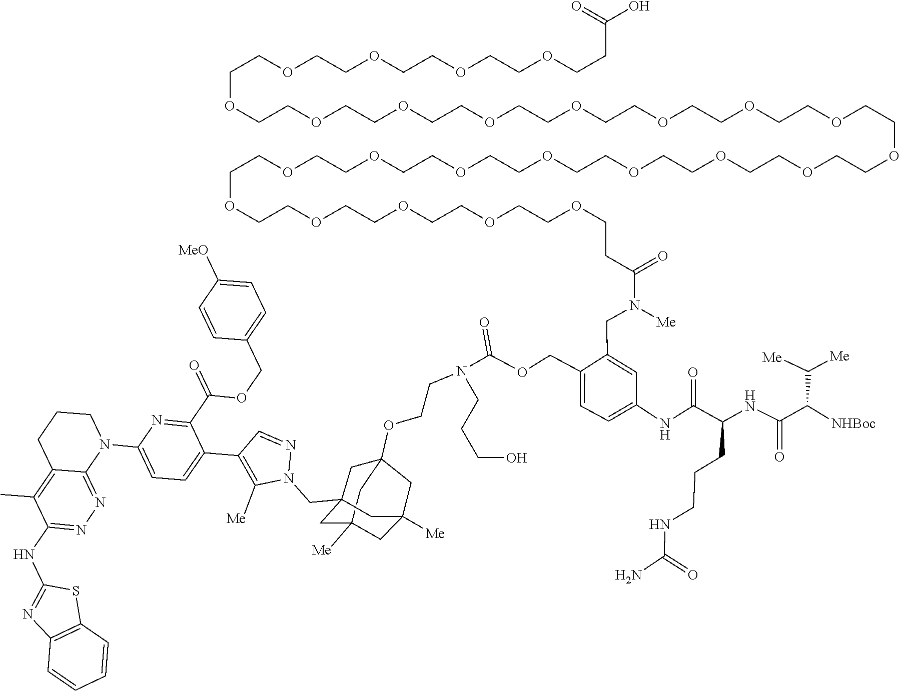 [SMILES: COc1ccc(COC(=O)c2nc(N3CCCc4c3nnc(Nc3nc5ccccc5s3)c4C)ccc2-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)OC(C)(C)C)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)cc1]

Following GENERAL PROCEDURE 3 with 4-methoxybenzyl 6-(3-(benzo[d]thiazol-2-ylamino)-4-methyl-6,7-dihydropyrido[2,3-c]pyridazin-8 (5H)-yl)-3-(1-(((1r,3s,5R,7S)-3-(2-((((4-((S)-2-((S)-2-((tert-butoxycarbonyl)amino)-3-methylbutanamido)-5-ureidopentanamido)-2-((methylamino)methyl)benzyl)oxy)carbonyl)(3-hydroxypropyl)amino)ethoxy)-5,7-dimethyladamantan-1-yl)methyl)-5-methyl-1H-pyrazol-4-yl) picolinate (32.0 mg, 0.022 mmol) and 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (43.3 mg, 0.033 mmol, 1.5 equiv.), 1-(2-((((2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)(3-hydroxypropyl) carbamoyl)oxy)methyl)-5-((S)-2-((S)-2-((tert-butoxycarbonyl)amino)-3-methylbutanamido)-5-ureidopentanamido)phenyl)-2-methyl-3-oxo-6,9,12,15,18,21,24,27,30,33,36,39,42,45,48,51,54,57,60,63,66,69,72,75,78-pentacosaoxa-2-azahenoctacontan-81-oic acid was obtained. HRMS: M−H+2Na=2705.3601; Rt=2.63 min (5 min acidic method).

### Synthesis of 3-(1-(((1r,3s,5R,7S)-3-(2-((((4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8 (5H)-yl)picolinic acid

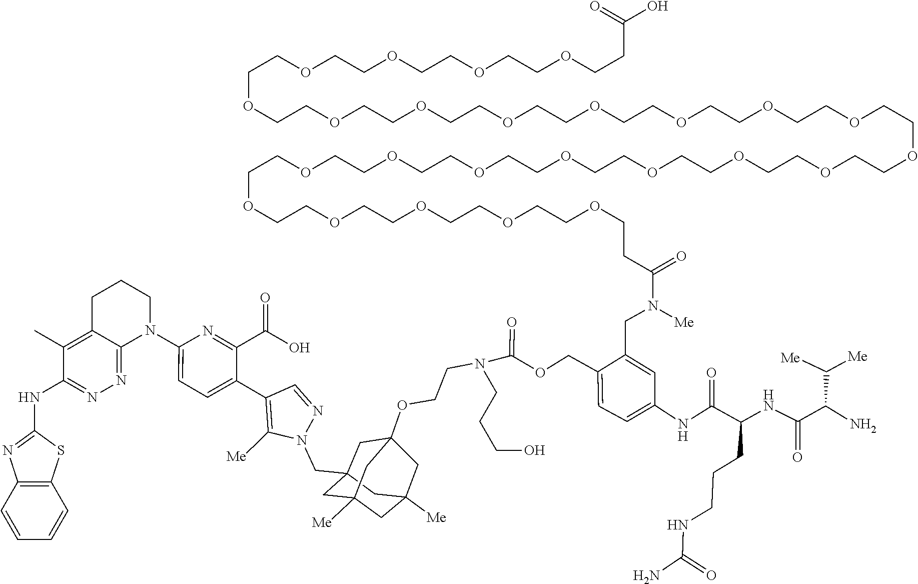 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](N)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

Following GENERAL PROCEDURE 4 with 1-(2-((((2-(((1s,3r,5R,7S)-3-((4-(6-(3-(benzo[d]thiazol-2-ylamino)-4-methyl-6,7-dihydropyrido[2,3-c]pyridazin-8 (5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)(3-hydroxypropyl) carbamoyl)oxy)methyl)-5-((S)-2-((S)-2-((tert-butoxycarbonyl)amino)-3-methylbutanamido)-5-ureidopentanamido)phenyl)-2-methyl-3-oxo-6,9,12,15,18,21,24,27,30,33,36,39,42,45,48,51,54,57,60,63,66,69,72,75,78-pentacosaoxa-2-azahenoctacontan-81-oic acid (35.1 mg, 0.013 mmol), 3-(1-(((1r,3s,5R,7S)-3-(2-((((4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8 (5H)-yl)picolinic acid was obtained. HRMS: M−H+2Na=2485.2700; Rt-2.02 min (5 min acidic method).

### Synthesis of 6-(3-(benzo[d]thiazol-2-ylamino)-4-methyl-6,7-dihydropyrido[2,3-c]pyridazin-8 (5H)-yl)-3-(1-(((1r,3s,5R,7S)-3-(2-((((2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(3-hydroxypropyl)amino)ethoxy)-5,7-dimethyladamantan-1-yl)methyl)-5-methyl-1H-pyrazol-4-yl)picolinic acid (L11C-P25)

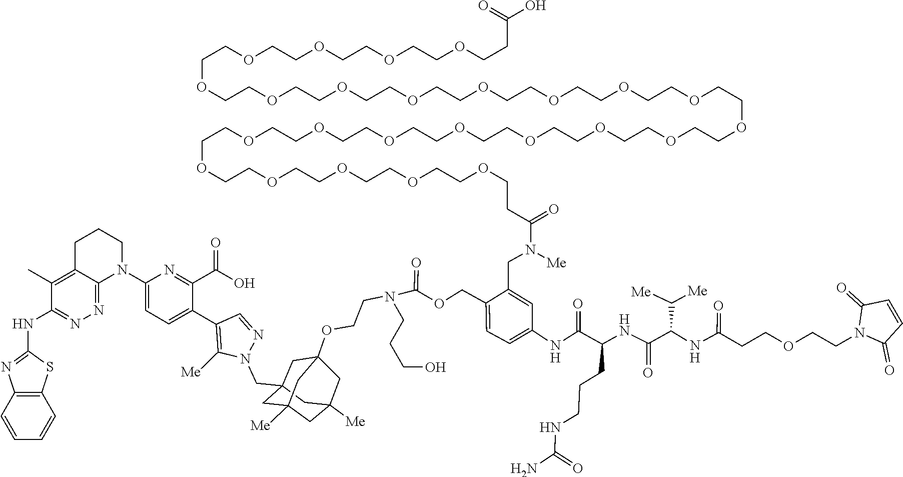 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

Following GENERAL PROCEDURE 5 with 3-(1-(((1r,3s,5R,7S)-3-(2-((((4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8 (5H)-yl)picolinic acid (17.3 mg, 0.007 mmol) and 2,5-dioxopyrrolidin-1-yl 3-(2-(2,5-dioxo-2,5-dihydro-1H-pyrrol-1-yl)ethoxy)propanoate (2.6 mg, 0.009 mmol, 1.2 equiv.), 6-(3-(benzo[d]thiazol-2-ylamino)-4-methyl-6,7-dihydropyrido[2,3-c]pyridazin-8 (5H)-yl)-3-(1-(((1r,3s,5R,7S)-3-(2-((((2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(3-hydroxypropyl)amino)ethoxy)-5,7-dimethyladamantan-1-yl)methyl)-5-methyl-1H-pyrazol-4-yl)picolinic acid was obtained. HRMS: M+H=2636.3701; Rt=1.73 min (5 min acidic method).

### Synthesis of 6-(3-(benzo[d]thiazol-2-ylamino)-4-methyl-6,7-dihydropyrido[2,3-c]pyridazin-8 (5H)-yl)-3-(1-(((1S,3s,5R,7S)-3-(2-((((4-((S)-2-((S)-2-((tert-butoxycarbonyl)amino)-3-methylbutanamido)-5-ureidopentanamido)-2-((methylamino)methyl)benzyl)oxy)carbonyl)(2-((S)-2,2-dimethyl-1,3-dioxolan-4-yl)ethyl)amino)ethoxy)-5,7-dimethyladamantan-1-yl)methyl)-5-methyl-1H-pyrazol-4-yl)picolinic acid

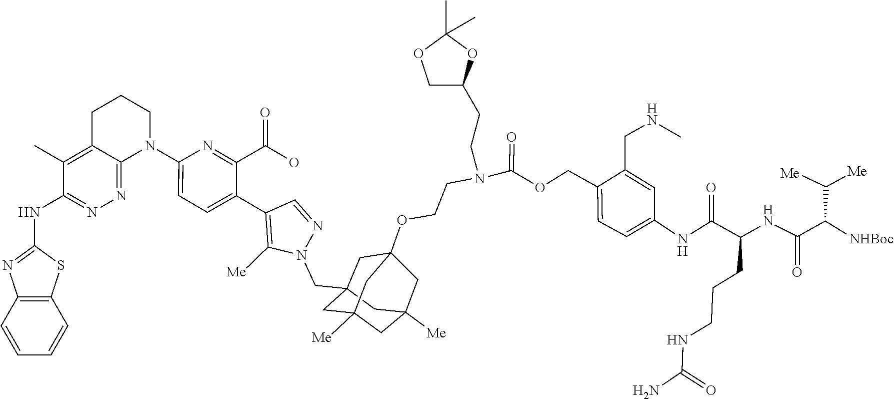 [SMILES: CNCc1cc(NC(=O)[C@H](CCCNC(N)=O)NC(=O)[C@@H](NC(=O)OC(C)(C)C)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H]1COC(C)(C)O1]

Following GENERAL PROCEDURE 9 with 6-(3-(benzo[d]thiazol-2-ylamino)-4-methyl-6,7-dihydropyrido[2,3-c]pyridazin-8 (5H)-yl)-3-(1-(((1S,3s,5R,7S)-3-(2-((2-((S)-2,2-dimethyl-1,3-dioxolan-4-yl)ethyl)amino)ethoxy)-5,7-dimethyladamantan-1-yl)methyl)-5-methyl-1H-pyrazol-4-yl)picolinic acid (24.0 mg, 0.028 mmol) and (9H-fluoren-9-yl)methyl (5-((S)-2-((S)-2-((tert-butoxycarbonyl)amino)-3-methylbutanamido)-5-ureidopentanamido)-2-((((4-nitrophenoxy)carbonyl)oxy)methyl)benzyl)(methyl)carbamate (27.9 mg, 0.031 mmol, 1.1 equiv.), 6-(3-(benzo[d]thiazol-2-ylamino)-4-methyl-6,7-dihydropyrido[2,3-c]pyridazin-8 (5H)-yl)-3-(1-(((1S,3s,5R,7S)-3-(2-((((4-((S)-2-((S)-2-((tert-butoxycarbonyl)amino)-3-methylbutanamido)-5-ureidopentanamido)-2-((methylamino)methyl)benzyl)oxy)carbonyl)(2-((S)-2,2-dimethyl-1,3-dioxolan-4-yl)ethyl)amino)ethoxy)-5,7-dimethyladamantan-1-yl)methyl)-5-methyl-1H-pyrazol-4-yl)picolinic acid was obtained. HRMS: M+H=1410.7300; Rt=2.24 min (5 min acidic method).

### Synthesis of 6-(3-(benzo[d]thiazol-2-ylamino)-4-methyl-6,7-dihydropyrido[2,3-c]pyridazin-8 (5H)-yl)-3-(1-(((1S,3s,5R,7S)-3-(2-((((4-((S)-2-((S)-2-((tert-butoxycarbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)oxy)carbonyl)(2-((S)-2,2-dimethyl-1,3-dioxolan-4-yl)ethyl)amino)ethoxy)-5,7-dimethyladamantan-1-yl)methyl)-5-methyl-1H-pyrazol-4-yl)picolinic acid

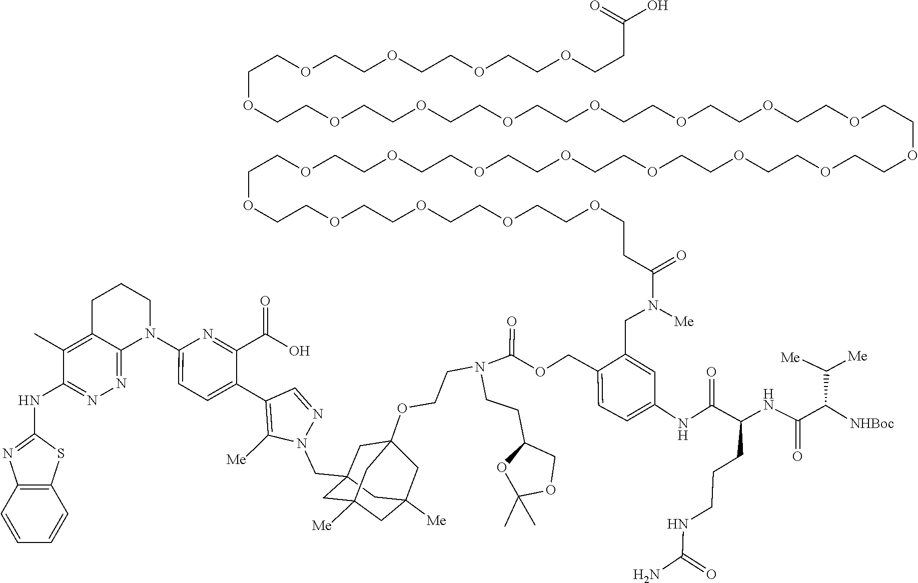 [SMILES: Cc1c(Nc2nc3ccccc3s2)nnc2c1CCCN2c1ccc(-c2cnn(CC34CC5(C)CC(C)(C3)CC(OCCN(CC[C@H]3COC(C)(C)O3)C(=O)OCc3ccc(NC(=O)[C@H](CCCNC(N)=O)NC(=O)[C@@H](NC(=O)OC(C)(C)C)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

Following GENERAL PROCEDURE 3 with 6-(3-(benzo[d]thiazol-2-ylamino)-4-methyl-6,7-dihydropyrido[2,3-c]pyridazin-8 (5H)-yl)-3-(1-(((1S,3s,5R,7S)-3-(2-((((4-((S)-2-((S)-2-((tert- butoxycarbonyl)amino)-3-methylbutanamido)-5-ureidopentanamido)-2-((methylamino)methyl)benzyl)oxy)carbonyl)(2-((S)-2,2-dimethyl-1,3-dioxolan-4-yl)ethyl)amino)ethoxy)-5,7-dimethyladamantan-1-yl)methyl)-5-methyl-1H-pyrazol-4-yl)picolinic acid (19.0 mg, 0.012 mmol) and 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (24.6 mg, 0.019 mmol, 1.5 equiv.), 6-(3-(benzo[d]thiazol-2-ylamino)-4-methyl-6,7-dihydropyrido[2,3-c]pyridazin-8 (5H)-yl)-3-(1-(((1S,3s,5R,7S)-3-(2-((((4-((S)-2-((S)-2-((tert-butoxycarbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)oxy)carbonyl)(2-((S)-2,2-dimethyl-1,3-dioxolan-4-yl)ethyl)amino)ethoxy)-5,7-dimethyladamantan-1-yl)methyl)-5-methyl-1H-pyrazol-4-yl)picolinic acid was obtained. HRMS: M−H+2Na=2655.3701; Rt=2.59 min (5 min acidic method).

### Synthesis of 3-(1-(((1S,3s,5R,7S)-3-(2-((((4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)oxy)carbonyl)((S)-3,4-dihydroxybutyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8 (5H)-yl)picolinic acid

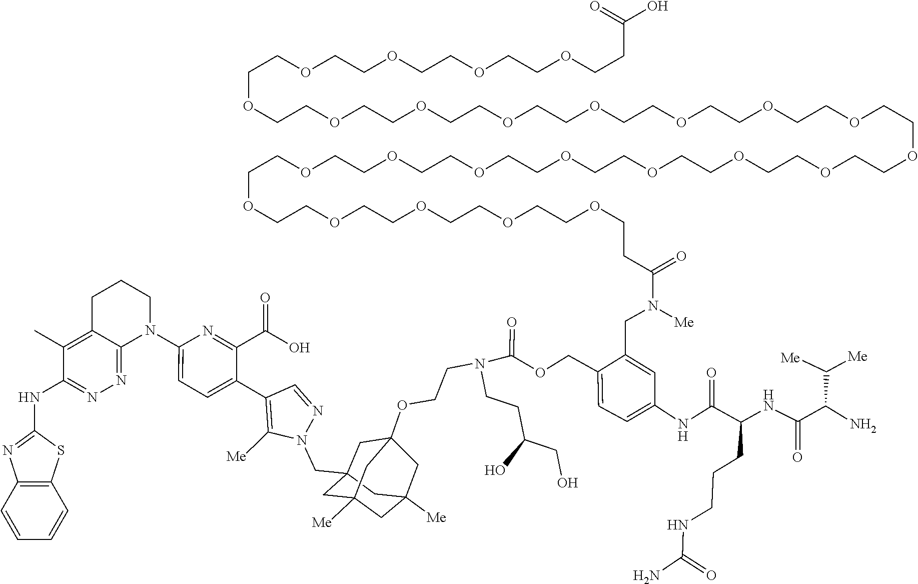 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](N)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

Following GENERAL PROCEDURE 4 with 6-(3-(benzo[d]thiazol-2-ylamino)-4-methyl-6,7-dihydropyrido[2,3-c]pyridazin-8 (5H)-yl)-3-(1-(((1S,3s,5R,7S)-3-(2-((((4-((S)-2-((S)-2-((tert- butoxycarbonyl)amino)-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)oxy)carbonyl)(2-((S)-2,2-dimethyl-1,3-dioxolan-4-yl)ethyl)amino)ethoxy)-5,7-dimethyladamantan-1-yl)methyl)-5-methyl-1H-pyrazol-4-yl)picolinic acid (28.4 mg, 0.011 mmol), 3-(1-(((1S,3s,5R,7S)-3-(2-((((4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)oxy)carbonyl)((S)-3,4-dihydroxybutyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8 (5H)-yl)picolinic acid was obtained. HRMS: M+H=2471.3301; Rt=1.97 min (5 min acidic method).

### Synthesis of 6-(3-(benzo[d]thiazol-2-ylamino)-4-methyl-6,7-dihydropyrido[2,3-c]pyridazin-8 (5H)-yl)-3-(1-(((1S,3s,5R,7S)-3-(2-((((2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)((S)-3,4-dihydroxybutyl)amino)ethoxy)-5,7-dimethyladamantan-1-yl)methyl)-5-methyl-1H-pyrazol-4-yl)picolinic acid (L11C-P19)

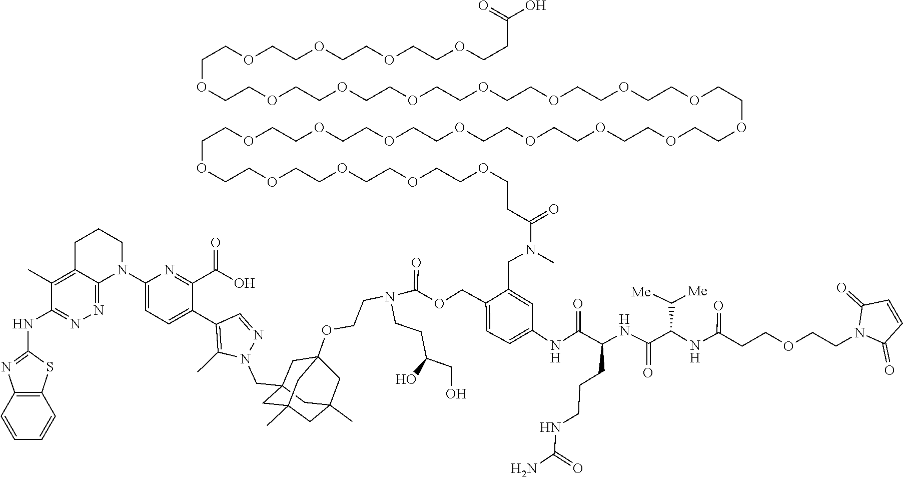 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

Following GENERAL PROCEDURE 5 with 3-(1-(((1S,3s,5R,7S)-3-(2-((((4-((S)-2-((S)-2-amino-3-methylbutanamido)-5-ureidopentanamido)-2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)benzyl)oxy)carbonyl)((S)-3,4-dihydroxybutyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8 (5H)-yl)picolinic acid (35.6 mg, 0.014 mmol) and 2,5-dioxopyrrolidin-1-yl 3-(2-(2,5-dioxo-2,5-dihydro-1H-pyrrol-1-yl)ethoxy)propanoate (10.7 mg, 0.034 mmol, 2.5 equiv.), 6-(3-(benzo[d]thiazol-2-ylamino)-4-methyl-6,7-dihydropyrido[2,3-c]pyridazin-8 (5H)-yl)-3-(1-(((1S,3s,5R,7S)-3-(2-((((2-(80-carboxy-2-methyl-3-oxo-6,9,12,15,18,21,24,27,30,33,36,39,42,45,48,51,54,57,60,63,66,69,72,75,78-pentacosaoxa-2-azaoctacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)((S)-3,4-dihydroxybutyl)amino)ethoxy)-5,7-dimethyladamantan-1-yl)methyl)-5-methyl-1H-pyrazol-4-yl)picolinic acid was obtained. HRMS: M+H=2666.3701; Rt=2.19 min (5 min acidic method).

### Synthesis of 5-(3-(4-(3-((((4-((S)-2-((S)-2-amino-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid

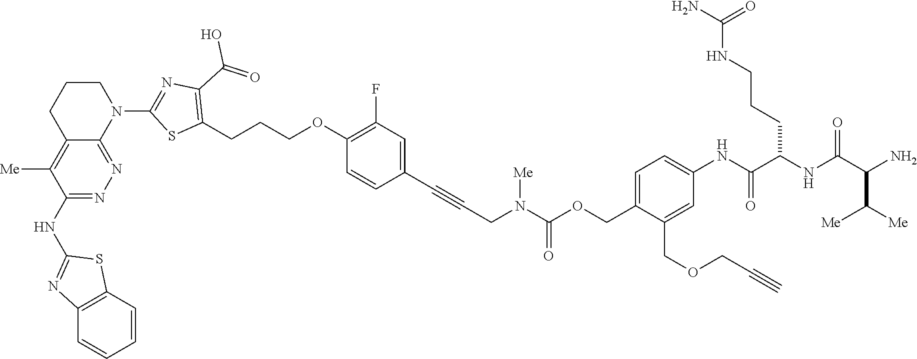 [SMILES: C#CCOCc1cc(NC(=O)[C@H](CCCNC(N)=O)NC(=O)[C@@H](N)C(C)C)ccc1COC(=O)N(C)CC#Cc1ccc(OCCCc2sc(N3CCCc4c3nnc(Nc3nc5ccccc5s3)c4C)nc2C(=O)O)c(F)c1]

Following GENERAL PROCEDURE 9 with 2-(3-(benzo[d]thiazol-2-ylamino)-4-methyl-6,7-dihydropyrido[2,3-c]pyridazin-8 (5H)-yl)-5-(3-(2-fluoro-4-(3-(methylamino)prop-1-yn-1-yl)phenoxy)propyl)thiazole-4-carboxylic acid (40.0 mg, 0.062 mmol) and (9H-fluoren-9-yl)methyl ((S)-3-methyl-1-(((S)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate (57.1 mg, 0.068 mmol, 1.1 equiv.), 5-(3-(4-(3-((((4-((S)-2-((S)-2-amino-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid was obtained. LCMS: M+H=1117.8; Rt=0.84 min (2 min acidic method).

### Synthesis of 5-(3-(4-(3-((((4-((S)-2-((S)-2-amino-3-methylbutanamido)-5-ureidopentanamido)-2-(((1-(26-carboxy-3,6,9,12,15,18,21,24-octaoxahexacosyl)-1H-1,2,3-triazol-4-yl)methoxy)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid

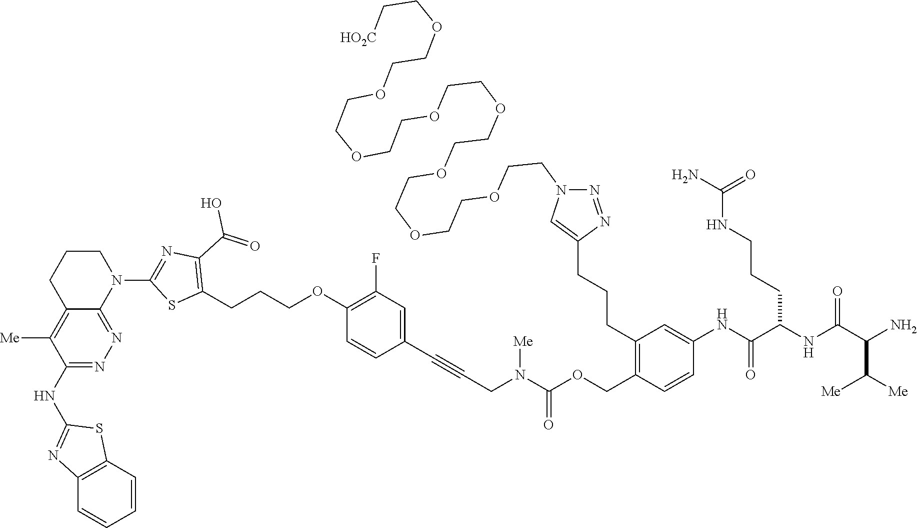 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](N)C(C)C)cc3CCCc3cn(CCOCCOCCOCCOCCOCCOCCOCCOCCC(=O)O)nn3)cc2F)s1]

Following GENERAL PROCEDURE 7 with 5-(3-(4-(3-((((4-((S)-2-((S)-2-amino-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid (18.2 mg, 0.016 mmol) and 1-azido-3,6,9,12,15,18,21,24-octaoxaheptacosan-27-oic acid (9.1 mg, 0.020 mmol, 1.2 equiv.), 5-(3-(4-(3-((((4-((S)-2-((S)-2-amino-3-methylbutanamido)-5-ureidopentanamido)-2-(((1-(26-carboxy-3,6,9,12,15,18,21,24-octaoxahexacosyl)-1H-1,2,3-triazol-4-yl)methoxy)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid was obtained. LCMS: M/2+H=793.1; Rt=1.17 min (2 min acidic method).

### Synthesis of 2-(3-(benzo[d]thiazol-2-ylamino)-4-methyl-6,7-dihydropyrido[2,3-c]pyridazin-8 (5H)-yl)-5-(3-(4-(3-((((2-(((1-(26-carboxy-3,6,9,12,15,18,21,24-octaoxahexacosyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazole-4-carboxylic acid (L7C-P3)

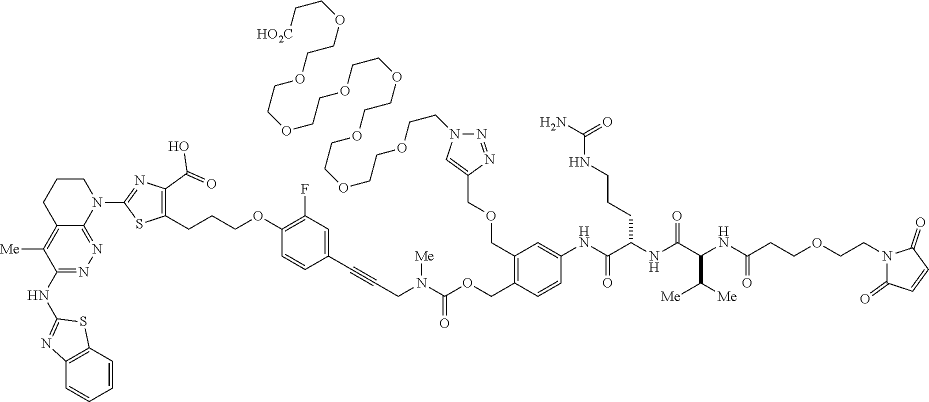 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3COCc3cn(CCOCCOCCOCCOCCOCCOCCOCCOCCC(=O)O)nn3)cc2F)s1]

Following GENERAL PROCEDURE 5 with 5-(3-(4-(3-((((4-((S)-2-((S)-2-amino-3-methylbutanamido)-5-ureidopentanamido)-2-(((1-(26-carboxy-3,6,9,12,15,18,21,24-octaoxahexacosyl)-1H-1,2,3-triazol-4-yl)methoxy)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid (10.5 mg, 0.007 mmol) and 2,5-dioxopyrrolidin-1-yl 3-(2-(2,5-dioxo-2,5-dihydro-1H-pyrrol-1-yl)ethoxy)propanoate (2.5 mg, 0.08 mmol, 1.2 equiv.), 2-(3-(benzo[d]thiazol-2-ylamino)-4-methyl-6,7-dihydropyrido[2,3-c]pyridazin-8 (5H)-yl)-5-(3-(4-(3-((((2-(((1-(26-carboxy-3,6,9,12,15,18,21,24-octaoxahexacosyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazole-4-carboxylic acid was obtained. LCMS: M/2+H=891.2; Rt=2.56 min (5 min acidic method).

### General Procedure 10

### Synthesis of 2-((6-(benzo[d]thiazol-2-ylamino)-5-methylpyridazin-3-yl)(3-(dimethylamino)propyl)amino)-5-(3-(4-(3-((((4-((S)-2-((S)-2-((tert-butoxycarbonyl)amino)-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)thiazole-4-carboxylic acid

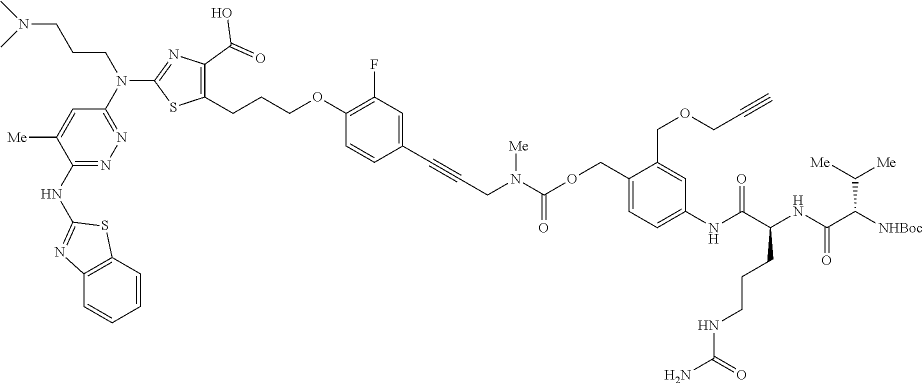 [SMILES: C#CCOCc1cc(NC(=O)[C@H](CCCNC(N)=O)NC(=O)[C@@H](NC(=O)OC(C)(C)C)C(C)C)ccc1COC(=O)N(C)CC#Cc1ccc(OCCCc2sc(N(CCCN(C)C)c3cc(C)c(Nc4nc5ccccc5s4)nn3)nc2C(=O)O)c(F)c1]

To a solution of 2-((6-(benzo[d]thiazol-2-ylamino)-5-methylpyridazin-3-yl)(3-(dimethylamino)propyl)amino)-5-(3-(2-fluoro-4-(3-(methylamino)prop-1-yn-1-yl)phenoxy)propyl)thiazole-4-carboxylic acid (30.0 mg, 0.039 mmol) and tert-butyl((R)-3-methyl-1-(((R)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate (36.5 mg, 0.051 mmol, 1.3 equiv.) in DMF (1.0 mL) was added DIPEA (0.034 mL, 0.197 mmol, 5.0 equiv.). The mixture was stirred for 2 hours at RT. DMSO (1.0 mL) was added and the solution was purified by RP-HPLC ISCO gold chromatography (0-100% MeCN/H2O, 0.1% TFA modifier). Upon lyophilization, 2-((6-(benzo[d]thiazol-2-ylamino)-5-methylpyridazin-3-yl)(3-(dimethylamino)propyl)amino)-5-(3-(4-(3-((((4-((S)-2-((S)-2-((tert-butoxycarbonyl)amino)-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)thiazole-4-carboxylic acid was obtained. HRMS: M+H=1262.5100; Rt=2.47 min (5 min acidic method).

### Synthesis of 2-((6-(benzo[d]thiazol-2-ylamino)-5-methylpyridazin-3-yl)(3-(dimethylamino)propyl)amino)-5-(3-(4-(3-((((4-((S)-2-((R)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)thiazole-4-carboxylic acid

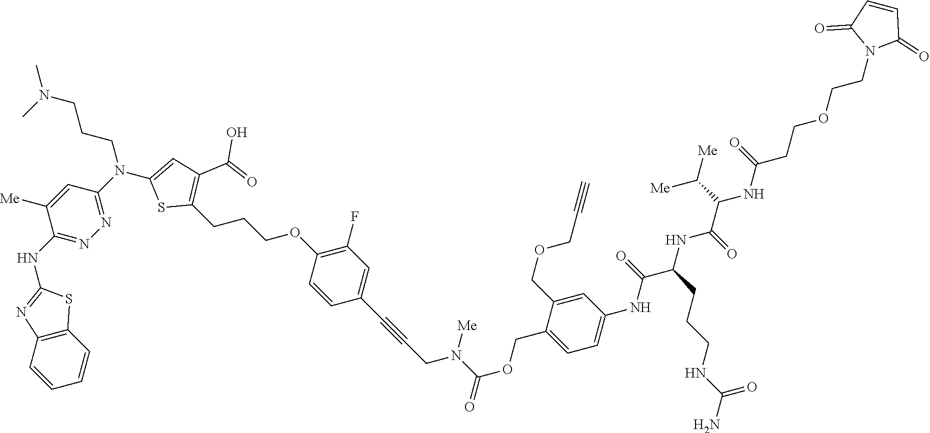

Following GENERAL PROCEDURE 8 with 2-((6-(benzo[d]thiazol-2-ylamino)-5-methylpyridazin-3-yl)(3-(dimethylamino)propyl)amino)-5-(3-(4-(3-((((4-((S)-2-((S)-2-((tert-butoxycarbonyl)amino)-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)thiazole-4-carboxylic acid (40.0 mg, 0.032 mmol) and 2,5-dioxopyrrolidin-1-yl 3-(2-(2,5-dioxo-2,5-dihydro-1H-pyrrol-1-yl)ethoxy)propanoate (11.8 mg, 0.038 mmol, 1.2 equiv.), 2-((6-(benzo[d]thiazol-2-ylamino)-5-methylpyridazin-3-yl)(3-(dimethylamino)propyl)amino)-5-(3-(4-(3-((((4-((S)-2-((R)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)thiazole-4-carboxylic acid was obtained. HRMS: M+H=1357.5262; Rt=1.16 min (2 min acidic method).

### Synthesis of 2-((6-(benzo[d]thiazol-2-ylamino)-5-methylpyridazin-3-yl)(3-(dimethylamino)propyl)amino)-5-(3-(4-(3-((((2-(((1-(26-carboxy-3,6,9,12,15,18,21,24-octaoxahexacosyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazole-4-carboxylic acid (L7C-P6)

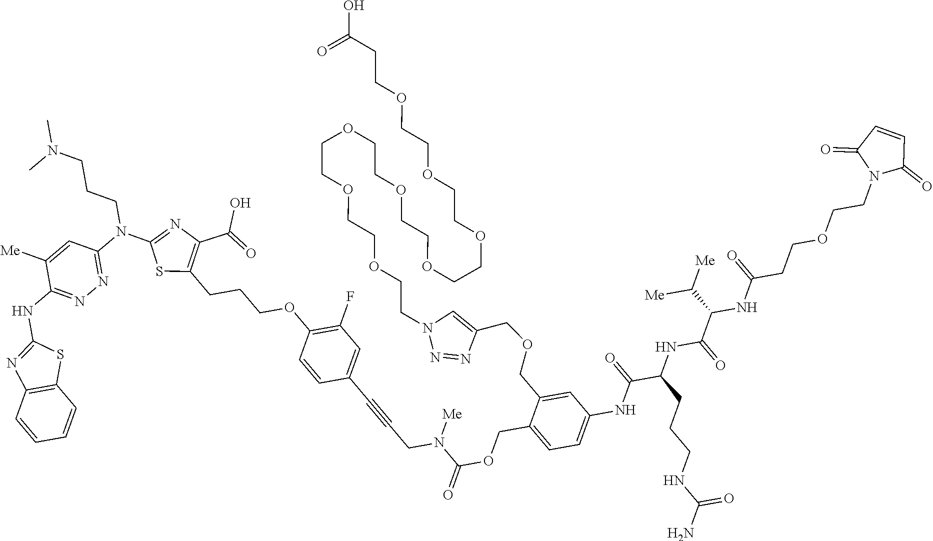 [SMILES: Cc1cc(N(CCCN(C)C)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4COCc4cn(CCOCCOCCOCCOCCOCCOCCOCCOCCC(=O)O)nn4)cc3F)s2)nnc1Nc1nc2ccccc2s1]

Following GENERAL PROCEDURE 7 with 2-((6-(benzo[d]thiazol-2-ylamino)-5-methylpyridazin-3-yl)(3-(dimethylamino)propyl)amino)-5-(3-(4-(3-((((4-((S)-2-((R)-2-(3-(2-(2,5-dioxo-2,5-dihydro-1H-pyrrol-1-yl)ethoxy)propanamido)-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)thiazole-4-carboxylic acid (10.0 mg, 0.008 mmol) and 1-azido-3,6,9,12,15,18,21,24-octaoxaheptacosan-27-oic acid (6.9 mg, 0.015 mmol, 2.0 equiv.), 2-((6-(benzo[d]thiazol-2-ylamino)-5-methylpyridazin-3-yl)(3-(dimethylamino)propyl)amino)-5-(3-(4-(3-((((2-(((1-(26-carboxy-3,6,9,12,15,18,21,24-octaoxahexacosyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazole-4-carboxylic acid was obtained. HRMS: M+H=1824.7700; Rt=2.19 min (5 min acidic method).

### Synthesis of 2-((6-(benzo[d]thiazol-2-ylamino)-5-methylpyridazin-3-yl)(methyl)amino)-5-(3-(4-(3-((((4-((S)-2-((S)-2-((tert-butoxycarbonyl)amino)-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)thiazole-4-carboxylic acid

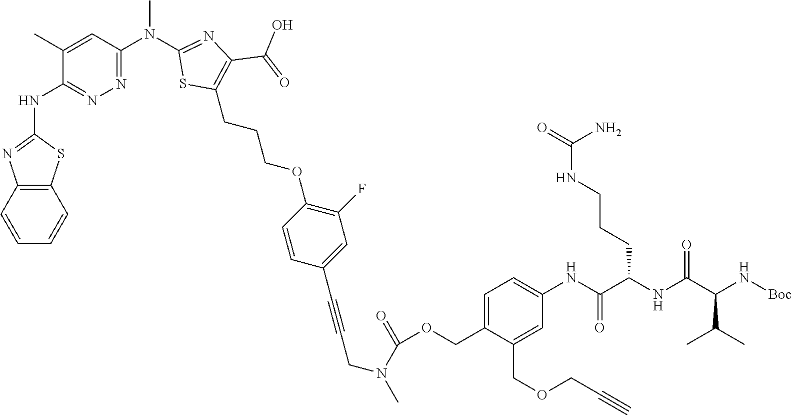 [SMILES: C#CCOCc1cc(NC(=O)[C@H](CCCNC(N)=O)NC(=O)[C@@H](NC(=O)OC(C)(C)C)C(C)C)ccc1COC(=O)N(C)CC#Cc1ccc(OCCCc2sc(N(C)c3cc(C)c(Nc4nc5ccccc5s4)nn3)nc2C(=O)O)c(F)c1]

Following GENERAL PROCEDURE 10 with 2-((6-(benzo[d]thiazol-2-ylamino)-5-methylpyridazin-3-yl)(methyl)amino)-5-(3-(2-fluoro-4-(3-(methylamino)prop-1-yn-1-yl)phenoxy)propyl)thiazole-4-carboxylic acid (50.0 mg, 0.081 mmol) and tert-butyl ((S)-3-methyl-1-(((S)-1-((4-((((4-nitrophenoxy)carbonyl)oxy)methyl)-3-((prop-2-yn-1-yloxy)methyl)phenyl)amino)-1-oxo-5-ureidopentan-2-yl)amino)-1-oxobutan-2-yl)carbamate (57.7 mg, 0.081 mmol, 1.0 equiv.), 2-((6-(benzo[d]thiazol-2-ylamino)-5-methylpyridazin-3-yl)(methyl)amino)-5-(3-(4-(3-((((4-((S)-2-((S)-2-((tert-butoxycarbonyl)amino)-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)thiazole-4-carboxylic acid was obtained. LCMS: M+H=1192.2; Rt=0.88 min (2 min basic method).

### Synthesis of 2-((6-(benzo[d]thiazol-2-ylamino)-5-methylpyridazin-3-yl)(methyl)amino)-5-(3-(4-(3-((((4-((S)-2-((S)-2-((tert-butoxycarbonyl)amino)-3-methylbutanamido)-5-ureidopentanamido)-2-(((1-(26-carboxy-3,6,9,12,15,18,21,24-octaoxahexacosyl)-1H-1,2,3-triazol-4-yl)methoxy)methyl)benzyl)oxy)carbonyl)(methyl)amino)prop-1-yn-1-yl)-2-fluorophenoxy)propyl)thiazole-4-carboxylic acid

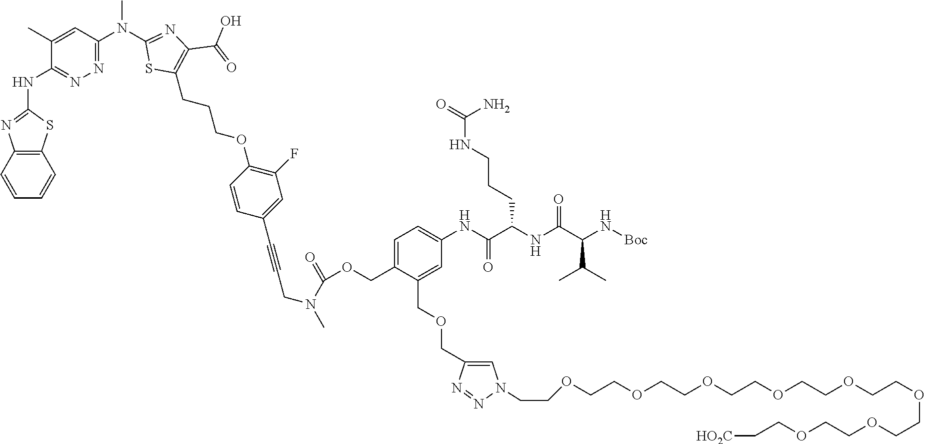 [SMILES: Cc1cc(N(C)c2nc(C(=O)O)c(CCCOc3ccc(C#CCN(C)C(=O)OCc4ccc(NC(=O)[C@H](CCCNC(N)=O)NC(=O)[C@@H](NC(=O)OC(C)(C)C)C(C)C)cc4COCc4cn(CCOCCOCCOCCOCCOCCOCCOCCOCCC(=O)O)nn4)cc3F)s2)nnc1Nc1nc2ccccc2s1]

Following GENERAL PROCEDURE 7 with 2-((6-(benzo[d]thiazol-2-ylamino)-5-methylpyridazin-3-yl)(methyl)amino)-5-(3-(4-(3-((((4-((S)-2-((S)-2-((tert-butoxycarbonyl)amino)-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)thiazole-4-carboxylic acid (38.0 mg, 0.032 mmol) and 1-azido-3,6,9,12,15,18,21,24-octaoxaheptacosan-27-oic acid (23.9 mg, 0.051 mmol, 1.6 equiv.), 2-((6-(benzo[d]thiazol-2-ylamino)-5-methylpyridazin-3-yl)(methyl)amino)-5-(3-(4-(3-((((4-((S)-2-((S)-2-((tert-butoxycarbonyl)amino)-3-methylbutanamido)-5-ureidopentanamido)-2-(((1-(26-carboxy-3,6,9,12,15,18,21,24-octaoxahexacosyl)-1H-1,2,3-triazol-4-yl)methoxy)methyl)benzyl)oxy)carbonyl)(methyl)amino)prop-1-yn-1-yl)-2-fluorophenoxy)propyl)thiazole-4-carboxylic acid was obtained. LCMS: M/2+H=830.6; Rt=0.73 min (2 min basic method).

### Synthesis of 2-((6-(benzo[d]thiazol-2-ylamino)-5-methylpyridazin-3-yl)(methyl)amino)-5-(3-(4-(3-((((2-(((1-(26-carboxy-3,6,9,12,15,18,21,24-octaoxahexacosyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazole-4-carboxylic acid (L7C-P7)

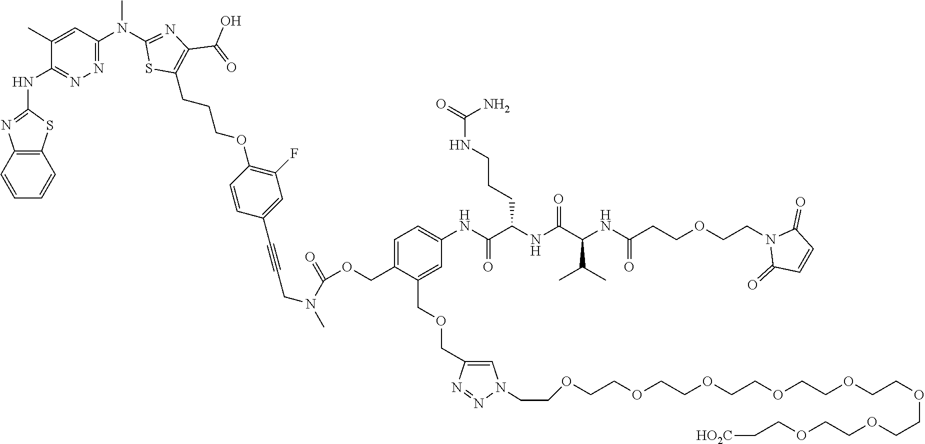 [SMILES: Cc1cc(N(C)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4COCc4cn(CCOCCOCCOCCOCCOCCOCCOCCOCCC(=O)O)nn4)cc3F)s2)nnc1Nc1nc2ccccc2s1]

Following GENERAL PROCEDURE 8 with 2-((6-(benzo[d]thiazol-2-ylamino)-5-methylpyridazin-3-yl)(methyl)amino)-5-(3-(4-(3-((((4-((S)-2-((S)-2-((tert-butoxycarbonyl)amino)-3-methylbutanamido)-5-ureidopentanamido)-2-(((1-(26-carboxy-3,6,9,12,15,18,21,24-octaoxahexacosyl)-1H-1,2,3-triazol-4-yl)methoxy)methyl)benzyl)oxy)carbonyl)(methyl)amino)prop-1-yn-1-yl)-2-fluorophenoxy)propyl)thiazole-4-carboxylic acid (25.0 mg, 0.015 mmol) and 2,5-dioxopyrrolidin-1-yl 3-(2-(2,5-dioxo-2,5-dihydro-1H-pyrrol-1-yl)ethoxy)propanoate (7.0 mg, 0.023 mmol, 1.5 equiv.), 2-((6-(benzo[d]thiazol-2-ylamino)-5-methylpyridazin-3-yl)(methyl)amino)-5-(3-(4-(3-((((2-(((1-(26-carboxy-3,6,9,12,15,18,21,24-octaoxahexacosyl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazole-4-carboxylic acid was obtained. LCMS: M/2+H=877.9; Rt=1.07 min (2 min acidic method).

### Synthesis of 5-(3-(4-(3-((((2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl)oxy)carbonyl)(methyl)amino)prop-1-yn-1-yl)-2-fluorophenoxy)propyl)-2-((6-(benzo[d]thiazol-2-ylamino)-5-methylpyridazin-3-yl)(methyl)amino)thiazole-4-carboxylic acid

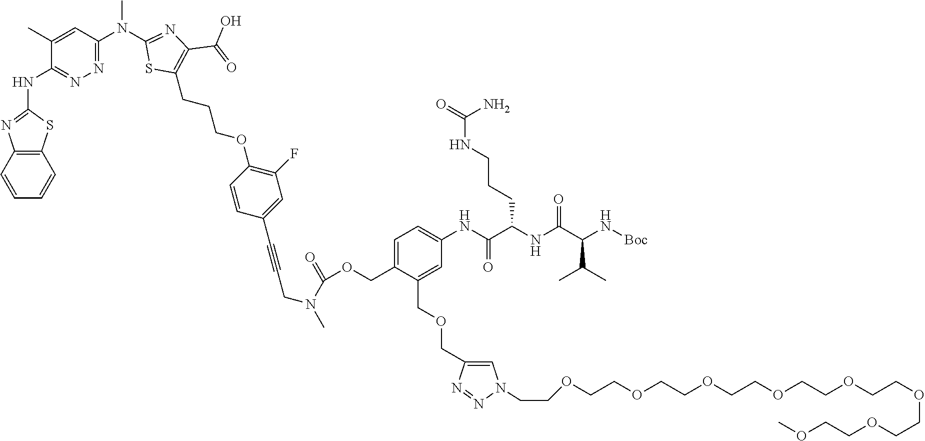 [SMILES: COCCOCCOCCOCCOCCOCCOCCOCCn1cc(COCc2cc(NC(=O)[C@H](CCCNC(N)=O)NC(=O)[C@@H](NC(=O)OC(C)(C)C)C(C)C)ccc2COC(=O)N(C)CC#Cc2ccc(OCCCc3sc(N(C)c4cc(C)c(Nc5nc6ccccc6s5)nn4)nc3C(=O)O)c(F)c2)nn1]

Following GENERAL PROCEDURE 7 with 2-((6-(benzo[d]thiazol-2-ylamino)-5-methylpyridazin-3-yl)(methyl)amino)-5-(3-(4-(3-((((4-((S)-2-((S)-2-((tert-butoxycarbonyl)amino)-3-methylbutanamido)-5-ureidopentanamido)-2-((prop-2-yn-1-yloxy)methyl)benzyl)oxy)carbonyl)(methyl)amino)prop-1-yn-1-yl)-2-fluorophenoxy)propyl)thiazole-4-carboxylic acid (45.0 mg, 0.038 mmol) and 25-azido-2,5,8,11,14,17,20,23-octaoxapentacosane (21.7 mg, 0.053 mmol, 1.4 equiv.), 5-(3-(4-(3-((((2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl)oxy)carbonyl)(methyl)amino)prop-1-yn-1-yl)-2-fluorophenoxy)propyl)-2-((6-(benzo[d]thiazol-2-ylamino)-5-methylpyridazin-3-yl)(methyl)amino)thiazole-4-carboxylic acid was obtained. LCMS: M/2+H=800.9; Rt=1.14 min (2 min acidic method).

### Synthesis of 5-(3-(4-(3-((((2-(((1-(2,5,8,11,14,17,20,23-octaoxapentacosan-25-yl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)-2-((6-(benzo[d]thiazol-2-ylamino)-5-methylpyridazin-3-yl)(methyl)amino)thiazole-4-carboxylic acid (L8C-P7)

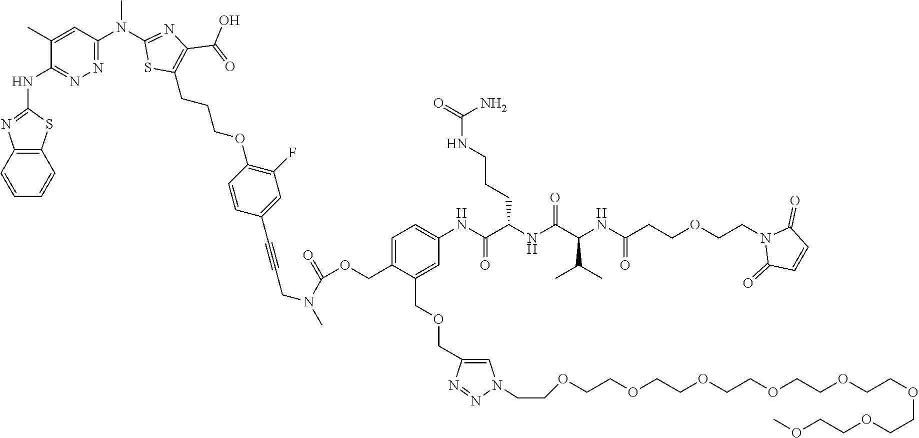 [SMILES: COCCOCCOCCOCCOCCOCCOCCOCCn1cc(COCc2cc(NC(=O)[C@H](CCCNC(N)=O)NC(=O)[C@@H](NC(=O)CCOCCN3C(=O)C=CC3=O)C(C)C)ccc2COC(=O)N(C)CC#Cc2ccc(OCCCc3sc(N(C)c4cc(C)c(Nc5nc6ccccc6s5)nn4)nc3C(=O)O)c(F)c2)nn1]

Following GENERAL PROCEDURE 8 with 5-(3-(4-(3-((((2-(((1-(2,5,8,11,14,17,20,23-octaoxapentacosan-25-yl)-1H-1,2,3-triazol-4-yl)methoxy)methyl)-4-((S)-2-((S)-2-((tert-butoxycarbonyl)amino)-3-methylbutanamido)-5-ureidopentanamido)benzyl)oxy)carbonyl)(methyl)amino)prop-1-yn-1-yl)-2-fluorophenoxy)propyl)-2-((6-(benzo[d]thiazol-2-ylamino)-5-methylpyridazin-3-yl)(methyl)amino)thiazole-4-carboxylic acid (49.0 mg, 0.031 mmol) and 2,5-dioxopyrrolidin-1-yl 3-(2-(2,5-dioxo-2,5-dihydro-1H-pyrrol-1-yl)ethoxy)propanoate (14.3 mg, 0.046 mmol, 1.5 equiv.), 5-(3-(4-(3-((((2-(((1-(2,5,8,11,14,17,20,23-octaoxapentacosan-25-yl)-1H-1,2,3-triazol-4-yl)methoxy)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)-2-((6-(benzo[d]thiazol-2-ylamino)-5-methylpyridazin-3-yl)(methyl)amino)thiazole-4-carboxylic acid was obtained. LCMS: M/2+H=848.6; Rt=1.08 min (2 min acidic method).

### Synthesis of 5-(3-(4-(3-((((4-((S)-2-((S)-2-amino-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid

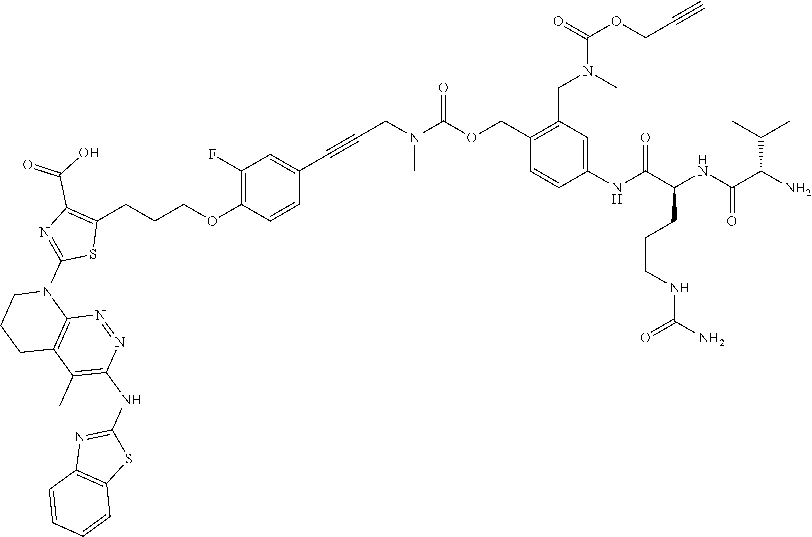 [SMILES: C#CCOC(=O)N(C)Cc1cc(NC(=O)[C@H](CCCNC(N)=O)NC(=O)[C@@H](N)C(C)C)ccc1COC(=O)N(C)CC#Cc1ccc(OCCCc2sc(N3CCCc4c3nnc(Nc3nc5ccccc5s3)c4C)nc2C(=O)O)c(F)c1]

Following GENERAL PROCEDURE 9 with prop-2-yn-1-yl 5-((S)-2-((S)-2-((((9H-fluoren-9-yl)methoxy)carbonyl)amino)-3-methylbutanamido)-5-ureidopentanamido)-2-((((4-nitrophenoxy)carbonyl)oxy)methyl)benzyl(methyl)carbamate (72.0 mg, 0.081 mmol) and 2-(3-(benzo[d]thiazol-2-ylamino)-4-methyl-6,7-dihydropyrido[2,3-c]pyridazin-8 (5H)-yl)-5-(3-(2-fluoro-4-(3-(methylamino)prop-1-yn-1-yl)phenoxy)propyl)thiazole-4-carboxylic acid (52.0 mg, 0.081 mmol, 1.0 equiv.), 5-(3-(4-(3-((((4-((S)-2-((S)-2-amino-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid was obtained. LCMS: M+H=1174.3; Rt=1.12 min (2 min acidic method).

### Synthesis of 5-(3-(4-(3-((((2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-amino-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid

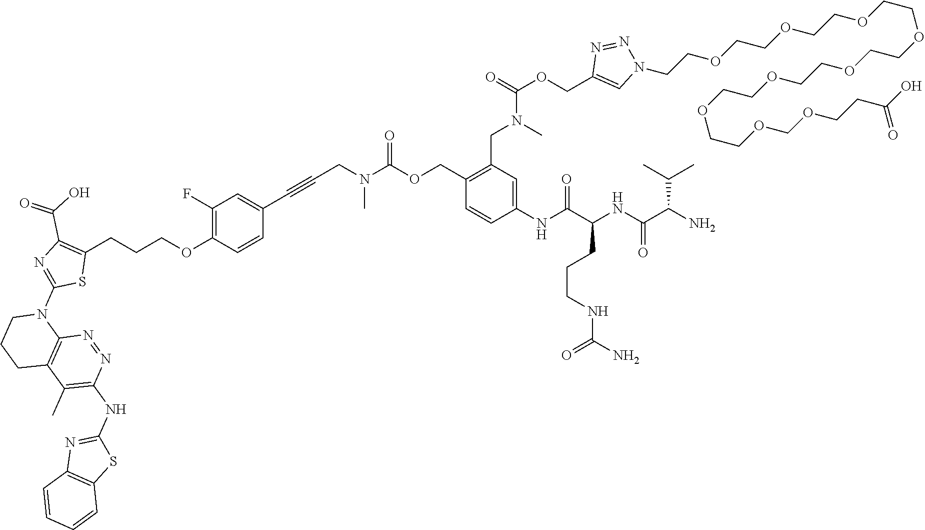 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](N)C(C)C)cc3CN(C)C(=O)OCc3cn(CCOCCOCCOCCOCCOCCOCCOCCOCOCCC(=O)O)nn3)cc2F)s1]

Following GENERAL PROCEDURE 7 with 5-(3-(4-(3-((((4-((S)-2-((S)-2-amino-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid (22.0 mg, 0.019 mmol) and 25-azido-2,5,8,11,14,17,20,23-octaoxapentacosane (23.0 mg, 0.056 mmol, 3.0 equiv.), 5-(3-(4-(3-((((2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-amino-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid was obtained. HRMS: M+H=1583.8199; Rt=2.30 min (5 min acidic method).

### Synthesis of 5-(3-(4-(3-((((2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid (L8C-P3)

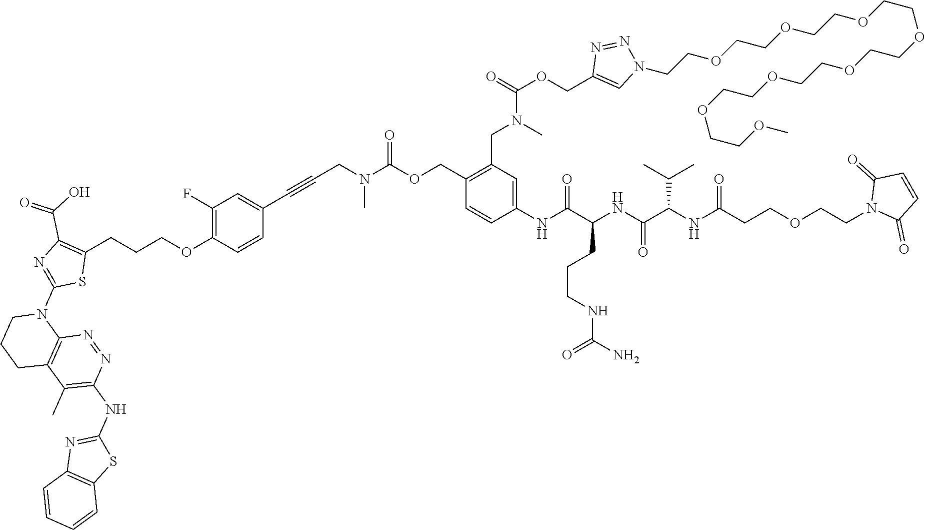 [SMILES: COCCOCCOCCOCCOCCOCCOCCOCCn1cc(COC(=O)N(C)Cc2cc(NC(=O)[C@H](CCCNC(N)=O)NC(=O)[C@@H](NC(=O)CCOCCN3C(=O)C=CC3=O)C(C)C)ccc2COC(=O)N(C)CC#Cc2ccc(OCCCc3sc(N4CCCc5c4nnc(Nc4nc6ccccc6s4)c5C)nc3C(=O)O)c(F)c2)nn1]

Following GENERAL PROCEDURE 5 with 5-(3-(4-(3-((((2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-amino-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid (12.3 mg, 0.008 mmol) and 2,5-dioxopyrrolidin-1-yl 3-(2-(2,5-dioxo-2,5-dihydro-1H-pyrrol-1-yl)ethoxy)propanoate (4.8 mg, 0.016 mmol, 2.0 equiv.), 5-(3-(4-(3-((((2-(((((1-(2,5,8,11,14,17,20,23-octaoxapentacosan-25-yl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid was obtained. HRMS: M+H=1778.6500; Rt=2.67 min (5 min acidic method).

### Synthesis of 5-(3-(4-(3-((((4-((S)-2-((S)-2-amino-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)(methyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid

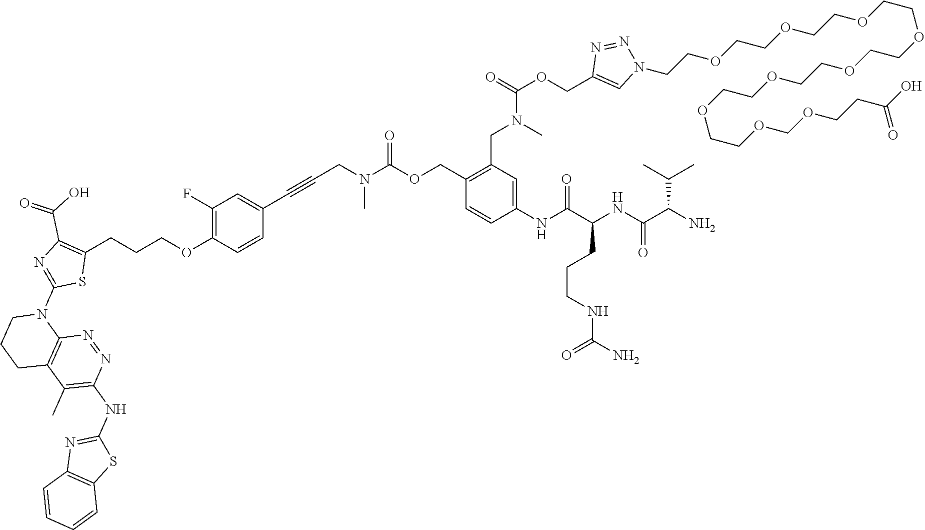 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](N)C(C)C)cc3CN(C)C(=O)OCc3cn(CCOCCOCCOCCOCCOCCOCCOCCOCOCCC(=O)O)nn3)cc2F)s1]

Following GENERAL PROCEDURE 7 with 5-(3-(4-(3-((((4-((S)-2-((S)-2-amino-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid (20.0 mg, 0.017 mmol) and 1-azido-3,6,9,12,15,18,21,24-octaoxaheptacosan-27-oic acid (23.9 mg, 0.051 mmol, 3.0 equiv.), 5-(3-(4-(3-((((4-((S)-2-((S)-2-amino-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)(methyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid was obtained. HRMS: M+H=1641.8900; Rt=2.24 min (5 min acidic method).

### Synthesis of 2-(3-(benzo[d]thiazol-2-ylamino)-4-methyl-6,7-dihydropyrido[2,3-c]pyridazin-8 (5H)-yl)-5-(3-(4-(3-((((2-(((((1-(26-carboxy-3,6,9,12,15,18,21,24-octaoxahexacosyl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazole-4-carboxylic acid (L3C-P3)

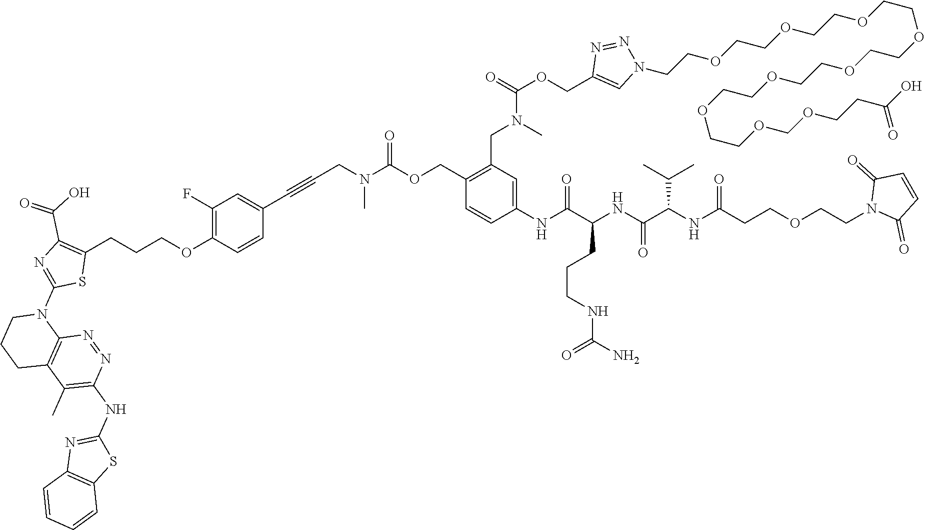 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3CN(C)C(=O)OCc3cn(CCOCCOCCOCCOCCOCCOCCOCCOCOCCC(=O)O)nn3)cc2F)s1]

Following GENERAL PROCEDURE 5 with 5-(3-(4-(3-((((4-((S)-2-((S)-2-amino-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)(methyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8 (5H)-yl)thiazole-4-carboxylic acid (5.5 mg, 0.003 mmol) and 2,5-dioxopyrrolidin-1-yl 3-(2-(2,5-dioxo-2,5-dihydro-1H-pyrrol-1-yl)ethoxy)propanoate (2.1 mg, 0.007 mmol, 2.0 equiv.), 2-(3-(benzo[d]thiazol-2-ylamino)-4-methyl-6,7-dihydropyrido[2,3-c]pyridazin-8 (5H)-yl)-5-(3-(4-(3-((((2-(((((1-(26-carboxy-3,6,9,12,15,18,21,24-octaoxahexacosyl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazole-4-carboxylic acid was obtained. HRMS: M+H=1837.6300; Rt=2.61 min (5 min acidic method).

### Synthesis of 5-((5-(3-(4-(3-((((4-((S)-2-((S)-2-amino-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl)-4-carboxythiazol-2-yl)(6-(benzo[d]thiazol-2-ylamino)-5-methylpyridazin-3-yl)amino)-2-hydroxy-N,N-dimethyl-N-(3-sulfopropyl) pentan-1-aminium

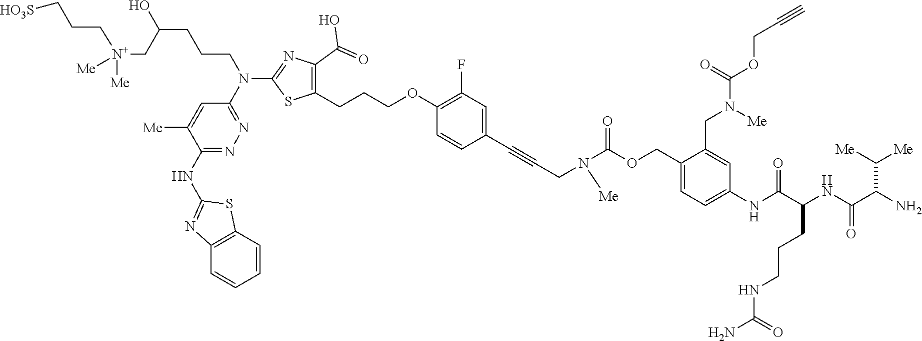 [SMILES: C#CCOC(=O)N(C)Cc1cc(NC(=O)[C@H](CCCNC(N)=O)NC(=O)[C@@H](N)C(C)C)ccc1COC(=O)N(C)CC#Cc1ccc(OCCCc2sc(N(CCCC(O)C[N+](C)(C)CCCS(=O)(=O)O)c3cc(C)c(Nc4nc5ccccc5s4)nn3)nc2C(=O)O)c(F)c1]

Following GENERAL PROCEDURE 9 with prop-2-yn-1-yl (5-((S)-2-((S)-2-((((9H-fluoren-9-yl)methoxy)carbonyl)amino)-3-methylbutanamido)-5-ureidopentanamido)-2-((((4-nitrophenoxy)carbonyl)oxy)methyl)benzyl)(methyl)carbamate (30.0 mg, 0.034 mmol) and 5-((6-(benzo[d]thiazol-2-ylamino)-5-methylpyridazin-3-yl)(4-carboxy-5-(3-(2-fluoro-4-(3-(methylamino)prop-1-yn-1-yl)phenoxy)propyl) thiazol-2-yl)amino)-2-hydroxy-N,N-dimethyl-N-(3-sulfopropyl) pentan-1-aminium (28.8 mg, 0.034 mmol, 1.0 equiv.), 5-((5-(3-(4-(3-((((4-((S)-2-((S)-2-amino-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl)-4-carboxythiazol-2-yl)(6-(benzo[d]thiazol-2-ylamino)-5-methylpyridazin-3-yl)amino)-2-hydroxy-N,N-dimethyl-N-(3-sulfopropyl) pentan-1-aminium was obtained. LCMS: M+H=1387.1; Rt=0.98 min (2 min acidic method).

### Synthesis of 5-((6-(benzo[d]thiazol-2-ylamino)-5-methylpyridazin-3-yl)(4-carboxy-5-(3-(4-(3-((((4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl) thiazol-2-yl)amino)-2-hydroxy-N,N-dimethyl-N-(3-sulfopropyl) pentan-1-aminium

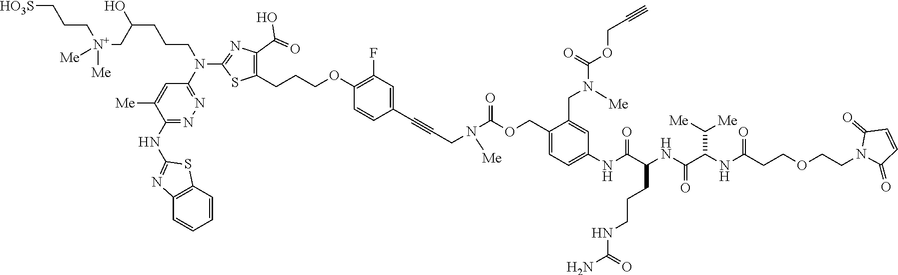 [SMILES: C#CCOC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(C)CC#Cc1ccc(OCCCc2sc(N(CCCC(O)C[N+](C)(C)CCCS(=O)(=O)O)c3cc(C)c(Nc4nc5ccccc5s4)nn3)nc2C(=O)O)c(F)c1]

Following GENERAL PROCEDURE 5 with 5-((5-(3-(4-(3-((((4-((S)-2-((S)-2-amino-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl)-4-carboxythiazol-2-yl)(6-(benzo[d]thiazol-2-ylamino)-5-methylpyridazin-3-yl)amino)-2-hydroxy-N,N-dimethyl-N-(3-sulfopropyl) pentan-1-aminium (35.6 mg, 0.026 mmol) and 2,5-dioxopyrrolidin-1-yl 3-(2-(2,5-dioxo-2,5-dihydro-1H-pyrrol-1-yl)ethoxy)propanoate (12.0 mg, 0.039 mmol, 1.5 equiv.), 5-((6-(benzo[d]thiazol-2-ylamino)-5-methylpyridazin-3-yl)(4-carboxy-5-(3-(4-(3-((((4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl) thiazol-2-yl)amino)-2-hydroxy-N,N-dimethyl-N-(3-sulfopropyl) pentan-1-aminium was obtained. LCMS: M/2+H=791.2; Rt=1.01 min (2 min acidic method).

### Synthesis of 5-((6-(benzo[d]thiazol-2-ylamino)-5-methylpyridazin-3-yl)(4-carboxy-5-(3-(4-(3-((((2-(((((1-(26-carboxy-3,6,9,12,15,18,21,24-octaoxahexacosyl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl) thiazol-2-yl)amino)-2-hydroxy-N,N-dimethyl-N-(3-sulfopropyl) pentan-1-aminium (L3C-P4)

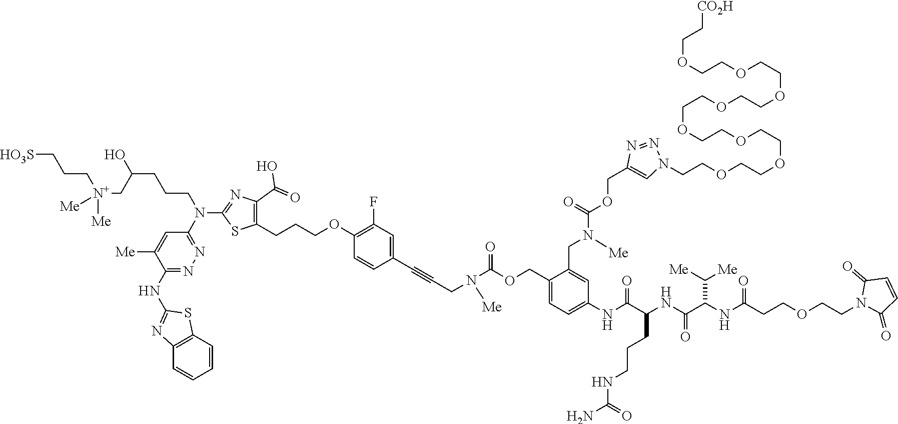 [SMILES: Cc1cc(N(CCCC(O)C[N+](C)(C)CCCS(=O)(=O)O)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4CN(C)C(=O)OCc4cn(CCOCCOCCOCCOCCOCCOCCOCCOCCC(=O)O)nn4)cc3F)s2)nnc1Nc1nc2ccccc2s1]

Following GENERAL PROCEDURE 7 with 5-((6-(benzo[d]thiazol-2-ylamino)-5-methylpyridazin-3-yl)(4-carboxy-5-(3-(4-(3-((((4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl) thiazol-2-yl)amino)-2-hydroxy-N,N-dimethyl-N-(3-sulfopropyl) pentan-1-aminium (23.0 mg, 0.015 mmol) and 1-azido-3,6,9,12,15,18,21,24-octaoxaheptacosan-27-oic acid (20.4 mg, 0.044 mmol, 3.0 equiv.), 5-((6-(benzo[d]thiazol-2-ylamino)-5-methylpyridazin-3-yl)(4-carboxy-5-(3-(4-(3-((((2-(((((1-(26-carboxy-3,6,9,12,15,18,21,24-octaoxahexacosyl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl) thiazol-2-yl)amino)-2-hydroxy-N,N-dimethyl-N-(3-sulfopropyl) pentan-1-aminium was obtained. HRMS: M+H=2047.8101; Rt=2.24 min (5 min acidic method).

### Synthesis of 2-((6-(benzo[d]thiazol-2-ylamino)-5-methylpyridazin-3-yl)(4-hydroxy-5-(trimethylammonio)pentyl)amino)-5-(3-(4-(3-((((4-((S)-2-((S)-2-((tert-butoxycarbonyl)amino)-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl)thiazole-4-carboxylate

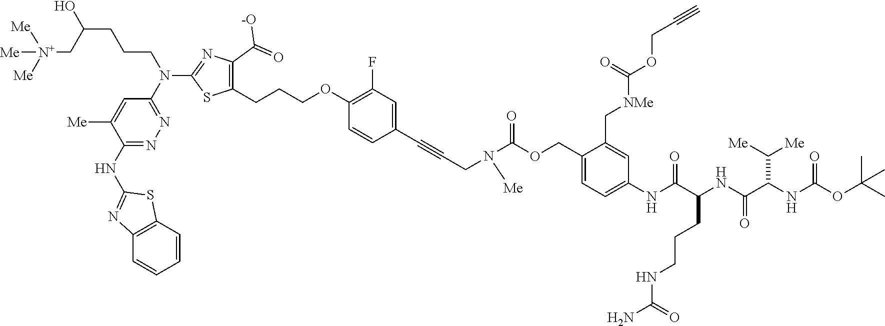

Following GENERAL PROCEDURE 10 with 2-((6-(benzo[d]thiazol-2-ylamino)-5-methylpyridazin-3-yl)(4-hydroxy-5-(trimethylammonio)pentyl)amino)-5-(3-(2-fluoro-4-(3-(methylamino)prop-1-yn-1-yl)phenoxy)propyl)thiazole-4-carboxylate (40.0 mg, 0.054 mmol) and prop-2-yn-1-yl 5-((S)-2-((S)-2-((tert-butoxycarbonyl)amino)-3-methylbutanamido)-5-ureidopentanamido)-2-((((4-nitrophenoxy)carbonyl)oxy)methyl)benzyl(methyl)carbamate (41.2 mg, 0.054 mmol, 1.0 equiv.), 2-((6-(benzo[d]thiazol-2-ylamino)-5-methylpyridazin-3-yl)(4-hydroxy-5-(trimethylammonio)pentyl)amino)-5-(3-(4-(3-((((4-((S)-2-((S)-2-((tert-butoxycarbonyl)amino)-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl)thiazole-4-carboxylate was obtained. LCMS: M+H=1378.1; Rt=1.11 min (2 min acidic method).

### Synthesis of 2-((6-(benzo[d]thiazol-2-ylamino)-5-methylpyridazin-3-yl)(4-hydroxy-5-(trimethylammonio)pentyl)amino)-5-(3-(4-(3-((((4-((S)-2-((S)-2-((tert-butoxycarbonyl)amino)-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)(methyl)amino)methyl)benzyl)oxy)carbonyl)(methyl)amino)prop-1-yn-1-yl)-2-fluorophenoxy)propyl)thiazole-4-carboxylate

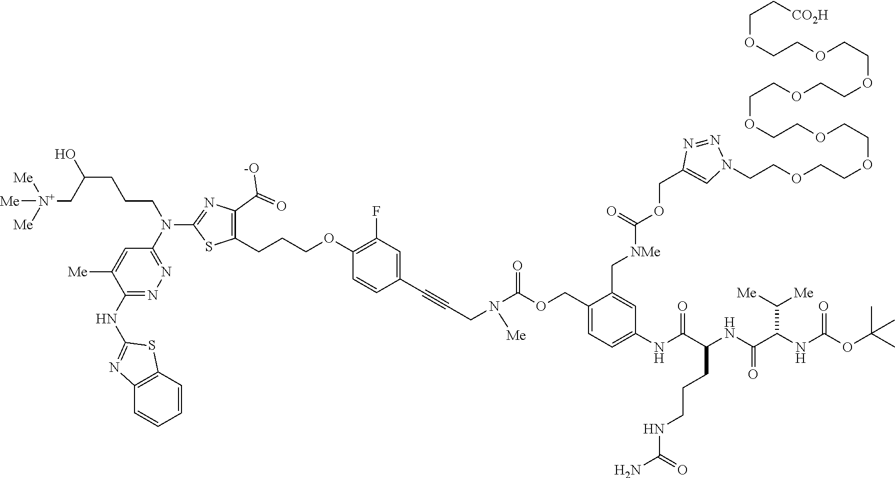

Following GENERAL PROCEDURE 7 with 2-((6-(benzo[d]thiazol-2-ylamino)-5-methylpyridazin-3-yl)(4-hydroxy-5-(trimethylammonio)pentyl)amino)-5-(3-(4-(3-((((4-((S)-2-((S)-2-((tert-butoxycarbonyl)amino)-3-methylbutanamido)-5-ureidopentanamido)-2-((methyl((prop-2-yn-1-yloxy)carbonyl)amino)methyl)benzyl)oxy)carbonyl)(methyl)amino)prop-1-yn-1-yl)-2-fluorophenoxy)propyl)thiazole-4-carboxylate (67.0 mg, 0.049 mmol) and 1-azido-3,6,9,12,15,18,21,24-octaoxaheptacosan-27-oic acid (34.1 mg, 0.073 mmol, 1.5 equiv.), 2-((6-(benzo[d]thiazol-2-ylamino)-5-methylpyridazin-3-yl)(4-hydroxy-5-(trimethylammonio)pentyl)amino)-5-(3-(4-(3-((((4-((S)-2-((S)-2-((tert-butoxycarbonyl)amino)-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)(methyl)amino)methyl)benzyl)oxy)carbonyl)(methyl)amino)prop-1-yn-1-yl)-2-fluorophenoxy)propyl)thiazole-4-carboxylate was obtained. LCMS: M/2+H=923.6; Rt=1.06 min (2 min acidic method).

### Synthesis of 5-(3-(4-(3-((((4-((S)-2-((S)-2-amino-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)(methyl)amino)methyl)benzyl)oxy)carbonyl)(methyl)amino)prop-1-yn-1-yl)-2-fluorophenoxy)propyl)-2-((6-(benzo[d]thiazol-2-ylamino)-5-methylpyridazin-3-yl)(4-hydroxy-5-(trimethylammonio)pentyl)amino)thiazole-4-carboxylate

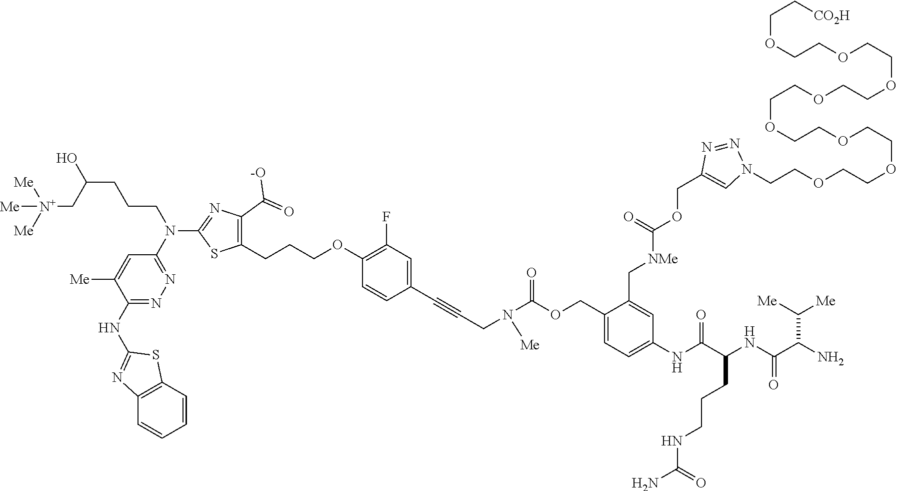

Following GENERAL PROCEDURE 4 with 2-((6-(benzo[d]thiazol-2-ylamino)-5-methylpyridazin-3-yl)(4-hydroxy-5-(trimethylammonio)pentyl)amino)-5-(3-(4-(3-((((4-((S)-2-((S)-2-((tert-butoxycarbonyl)amino)-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)(methyl)amino)methyl)benzyl)oxy)carbonyl)(methyl)amino)prop-1-yn-1-yl)-2-fluorophenoxy)propyl)thiazole-4-carboxylate (53.7 mg, 0.029 mmol), 5-(3-(4-(3-((((4-((S)-2-((S)-2-amino-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)(methyl)amino)methyl)benzyl)oxy)carbonyl)(methyl)amino)prop-1-yn-1-yl)-2-fluorophenoxy)propyl)-2-((6-(benzo[d]thiazol-2-ylamino)-5-methylpyridazin-3-yl)(4-hydroxy-5-(trimethylammonio)pentyl)amino)thiazole-4-carboxylate was obtained. LCMS: M/2+H=873.5; Rt=1.03 min (2 min acidic method).

### Synthesis of 5-((6-(benzo[d]thiazol-2-ylamino)-5-methylpyridazin-3-yl)(4-carboxy-5-(3-(4-(3-((((2-(((((1-(26-carboxy-3,6,9,12,15,18,21,24-octaoxahexacosyl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl) thiazol-2-yl)amino)-2-hydroxy-N,N,N-trimethylpentan-1-aminium (L3C-P5)

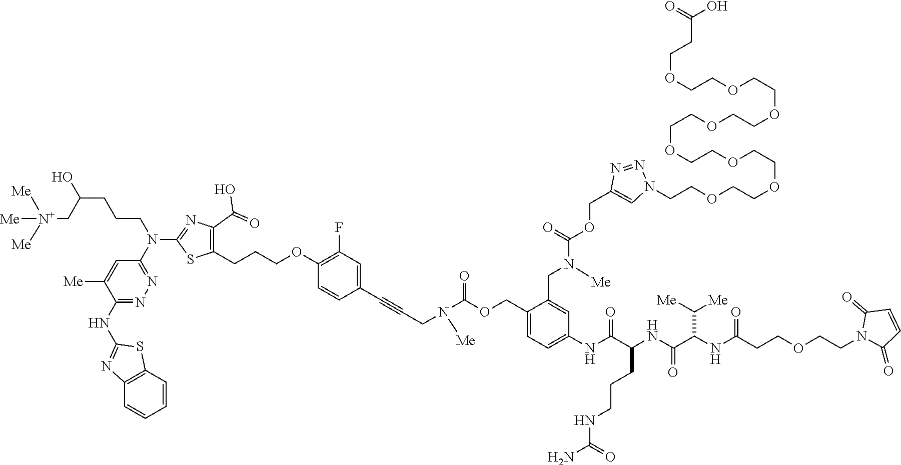 [SMILES: Cc1cc(N(CCCC(O)C[N+](C)(C)C)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4CN(C)C(=O)OCc4cn(CCOCCOCCOCCOCCOCCOCCOCCOCCC(=O)O)nn4)cc3F)s2)nnc1Nc1nc2ccccc2s1]

Following GENERAL PROCEDURE 5 with 5-(3-(4-(3-((((4-((S)-2-((S)-2-amino-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)(methyl)amino)methyl)benzyl)oxy)carbonyl)(methyl)amino)prop-1-yn-1-yl)-2-fluorophenoxy)propyl)-2-((6-(benzo[d]thiazol-2-ylamino)-5-methylpyridazin-3-yl)(4-hydroxy-5-(trimethylammonio)pentyl)amino)thiazole-4-carboxylate (50.8 mg, 0.029 mmol) and 2,5-dioxopyrrolidin-1-yl 3-(2-(2,5-dioxo-2,5-dihydro-1H-pyrrol-1-yl)ethoxy)propanoate (13.6 mg, 0.044 mmol, 1.5 equiv.), 5-((6-(benzo[d]thiazol-2-ylamino)-5-methylpyridazin-3-yl)(4-carboxy-5-(3-(4-(3-((((2-(((((1-(26-carboxy-3,6,9,12,15,18,21,24-octaoxahexacosyl)-1H-1,2,3-triazol-4-yl)methoxy)carbonyl)(methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl) thiazol-2-yl)amino)-2-hydroxy-N,N,N-trimethylpentan-1-aminium was obtained. HRMS: M+H=1939.8199; Rt=2.17 min (5 min acidic method).

### Synthesis of 2-(3-(benzo[d]thiazol-2-ylamino)-4-methyl-6,7-dihydropyrido[2,3-c]pyridazin-8(5H)-yl)-5-(3-(4-(3-((((4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl)oxy)carbonyl)(methyl)amino)prop-1-yn-1-yl)-2-fluorophenoxy)propyl)thiazole-4-carboxylic acid

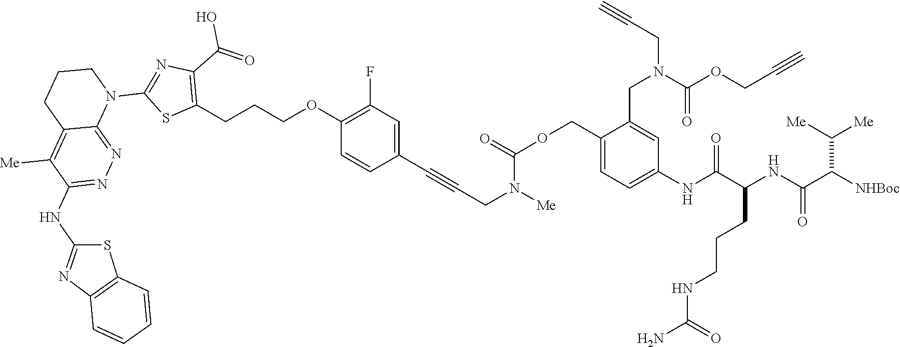 [SMILES: C#CCOC(=O)N(CC#C)Cc1cc(NC(=O)[C@H](CCCNC(N)=O)NC(=O)[C@@H](NC(=O)OC(C)(C)C)C(C)C)ccc1COC(=O)N(C)CC#Cc1ccc(OCCCc2sc(N3CCCc4c3nnc(Nc3nc5ccccc5s3)c4C)nc2C(=O)O)c(F)c1]

Following GENERAL PROCEDURE 10 with prop-2-yn-1-yl 5-((S)-2-((S)-2-((tert-butoxycarbonyl)amino)-3-methylbutanamido)-5-ureidopentanamido)-2-((((4-nitrophenoxy)carbonyl)oxy)methyl)benzyl(prop-2-yn-1-yl) carbamate (49.3 mg, 0.062 mmol) and 2-(3-(benzo[d]thiazol-2-ylamino)-4-methyl-6,7-dihydropyrido[2,3-c]pyridazin-8(5H)-yl)-5-(3-(2-fluoro-4-(3-(methylamino)prop-1-yn-1-yl) phenoxy)propyl)thiazole-4-carboxylic (40.0 mg, 0.062 mmol, 1.0 equiv.), 2-(3-(benzo[d]thiazol-2-ylamino)-4-methyl-6,7-dihydropyrido[2,3-c]pyridazin-8(5H)-yl)-5-(3-(4-(3-((((4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl)oxy)carbonyl)(methyl)amino)prop-1-yn-1-yl)-2-fluorophenoxy)propyl)thiazole-4-carboxylic acid was obtained. LCMS: M+H=1297.0; Rt=1.28 min (2 min acidic method).

### Synthesis of 5-(3-(4-(3-((((2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-((tert-butoxycarbonyl)amino)-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid

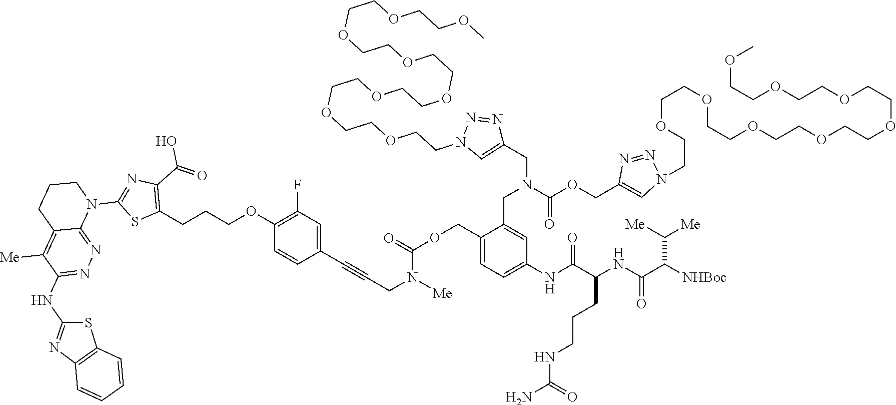 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](NC(=O)OC(C)(C)C)C(C)C)ccc2COC(=O)N(C)CC#Cc2ccc(OCCCc3sc(N4CCCc5c4nnc(Nc4nc6ccccc6s4)c5C)nc3C(=O)O)c(F)c2)nn1]

Following GENERAL PROCEDURE 7 with 2-(3-(benzo[d]thiazol-2-ylamino)-4-methyl-6,7-dihydropyrido[2,3-c]pyridazin-8(5H)-yl)-5-(3-(4-(3-((((4-((S)-2-((S)-2-((tert-butoxycarbonyl)amino)-3-methylbutanamido)-5-ureidopentanamido)-2-((prop-2-yn-1-yl((prop-2-yn-1-yloxy)carbonyl)amino)methyl)benzyl)oxy)carbonyl)(methyl)amino)prop-1-yn-1-yl)-2-fluorophenoxy)propyl)thiazole-4-carboxylic acid (49.0 mg, 0.038 mmol) and 25-azido-2,5,8,11,14,17,20,23-octaoxapentacosane (34.0 mg, 0.083 mmol, 2.2 equiv.), 5-(3-(4-(3-((((2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-((tert-butoxycarbonyl)amino)-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid was obtained. LCMS: M/2+H=1059.4; Rt=1.16 min (2 min acidic method).

### Synthesis of 5-(3-(4-(3-((((2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-amino-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid

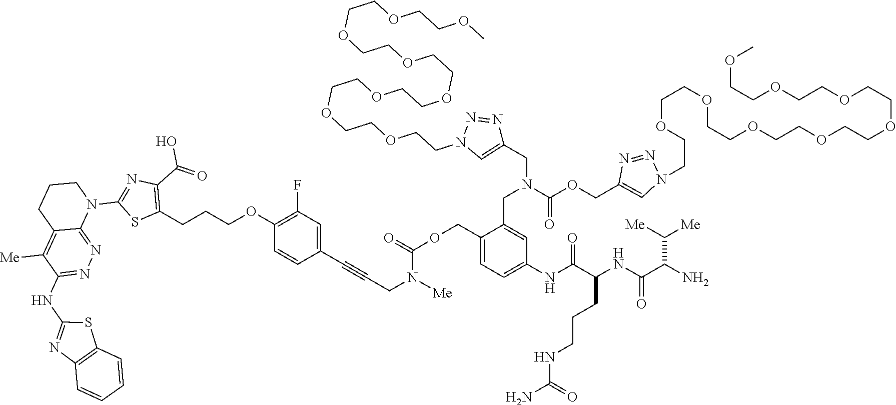 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](N)C(C)C)ccc2COC(=O)N(C)CC#Cc2ccc(OCCCc3sc(N4CCCc5c4nnc(Nc4nc6ccccc6s4)c5C)nc3C(=O)O)c(F)c2)nn1]

Following GENERAL PROCEDURE 4 with 5-(3-(4-(3-((((2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-((tert-butoxycarbonyl)amino)-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid (80.0 mg, 0.038 mmol), 5-(3-(4-(3-((((2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-amino-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid was obtained. LCMS: M/2+H=1009.2; Rt=1.14 min (2 min acidic method).

### Synthesis of 5-(3-(4-(3-((((2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid (L1C-P3)

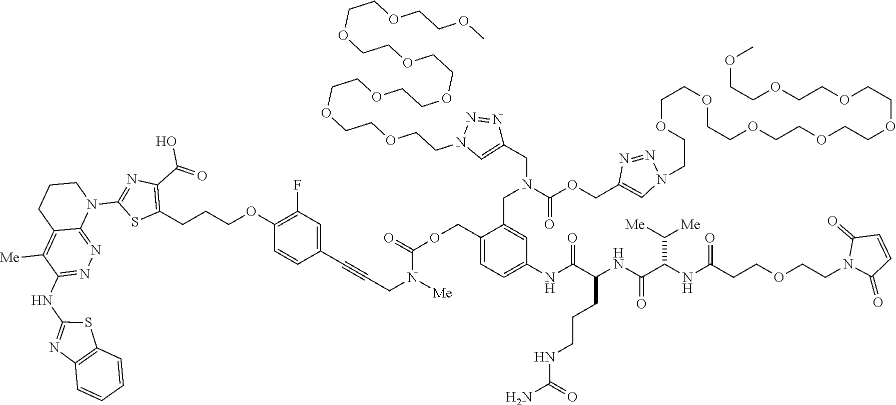 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](NC(=O)CCOCCN3C(=O)C=CC3=O)C(C)C)ccc2COC(=O)N(C)CC#Cc2ccc(OCCCc3sc(N4CCCc5c4nnc(Nc4nc6ccccc6s4)c5C)nc3C(=O)O)c(F)c2)nn1]

Following GENERAL PROCEDURE 5 with 5-(3-(4-(3-((((2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-amino-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid (76.0 mg, 0.038 mmol) and 2,5-dioxopyrrolidin-1-yl 3-(2-(2,5-dioxo-2,5-dihydro-1H-pyrrol-1-yl)ethoxy)propanoate (23.4 mg, 0.075 mmol, 2.0 equiv.), 5-(3-(4-(3-((((2-(((((1-(2,5,8,11,14,17,20,23-octaoxapentacosan-25-yl)-1H-1,2,3-triazol-4-yl)methoxy)carbonyl)((1-(2,5,8,11,14,17,20,23-octaoxapentacosan-25-yl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid was obtained. HRMS: M+H=2211.9700; Rt=2.56 min (5 min acidic method).

### Synthesis of 5-(3-(4-(3-((((4-((S)-2-((S)-2-((((9H-fluoren-9-yl)methoxy)carbonyl)amino)-3-methylbutanamido)-5-ureidopentanamido)-2-((prop-2-yn-1-yl((prop-2-yn-1-yloxy)carbonyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid

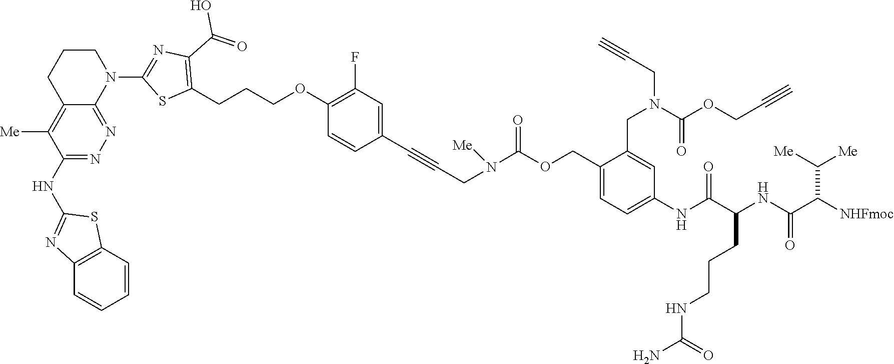

Following GENERAL PROCEDURE 10 with prop-2-yn-1-yl 5-((S)-2-((S)-2-((((9H-fluoren-9-yl)methoxy)carbonyl)amino)-3-methylbutanamido)-5-ureidopentanamido)-2-((((4-nitrophenoxy)carbonyl)oxy)methyl)benzyl(prop-2-yn-1-yl) carbamate (56.9 mg, 0.062 mmol) and 2-(3-(benzo[d]thiazol-2-ylamino)-4-methyl-6,7-dihydropyrido[2,3-c]pyridazin-8(5H)-yl)-5-(3-(2-fluoro-4-(3-(methylamino)prop-1-yn-1-yl) phenoxy)propyl)thiazole-4-carboxylic acid (40.0 mg, 0.062 mmol, 1.0 equiv.), 5-(3-(4-(3-((((4-((S)-2-((S)-2-((((9H-fluoren-9-yl)methoxy)carbonyl)amino)-3-methylbutanamido)-5-ureidopentanamido)-2-((prop-2-yn-1-yl((prop-2-yn-1-yloxy)carbonyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid was obtained. LCMS: M+H=1422.6; Rt=1.33 min (2 min acidic method).

### Synthesis of 5-(3-(4-(3-((((4-((S)-2-((S)-2-amino-3-methylbutanamido)-5-ureidopentanamido)-2-((prop-2-yn-1-yl((prop-2-yn-1-yloxy)carbonyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid

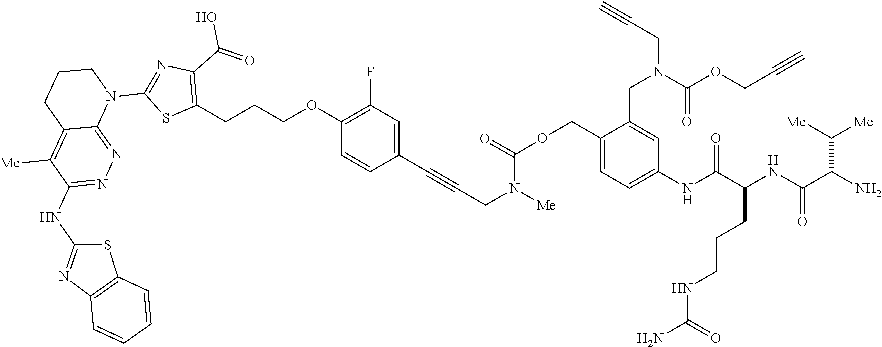 [SMILES: C#CCOC(=O)N(CC#C)Cc1cc(NC(=O)[C@H](CCCNC(N)=O)NC(=O)[C@@H](N)C(C)C)ccc1COC(=O)N(C)CC#Cc1ccc(OCCCc2sc(N3CCCc4c3nnc(Nc3nc5ccccc5s3)c4C)nc2C(=O)O)c(F)c1]

A solution of 5-(3-(4-(3-((((4-((S)-2-((S)-2-((((9H-fluoren-9-yl)methoxy)carbonyl)amino)-3-methylbutanamido)-5-ureidopentanamido)-2-((prop-2-yn-1-yl((prop-2-yn-1-yloxy)carbonyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid (88.0 mg, 0.062 mmol) in 2.0 M dimethylamine in THF (3.1 mL, 6.20 mmol, 100.0 equiv.) was stirred for 80 min. at RT. The solvents were removed in vacuo, diluted residue in DMSO (1.0 mL) and purified by RP-HPLC ISCO gold chromatography (0-100% MeCN/H2O, 0.05% TFA modifier). Upon lyophilization, 5-(3-(4-(3-((((4-((S)-2-((S)-2-amino-3-methylbutanamido)-5-ureidopentanamido)-2-((prop-2-yn-1-yl((prop-2-yn-1-yloxy)carbonyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid was obtained. LCMS: M+H=1199.2; Rt=1.06 min (2 min acidic method).

### Synthesis of 5-(3-(4-(3-((((4-((S)-2-((S)-2-amino-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)((1-(26-carboxy-3,6,9,12,15,18,21,24-octaoxahexacosyl)-1H-1,2,3-triazol-4-yl)methyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid

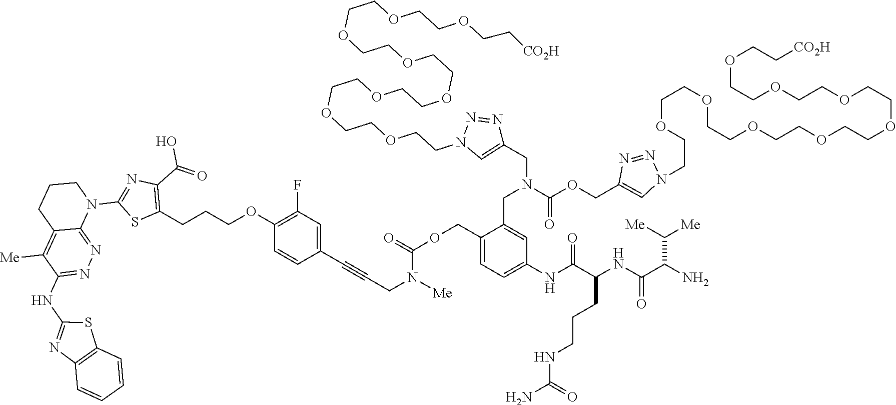 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](N)C(C)C)cc3CN(Cc3cn(CCOCCOCCOCCOCCOCCOCCOCCOCCC(=O)O)nn3)C(=O)OCc3cn(CCOCCOCCOCCOCCOCCOCCOCCOCCC(=O)O)nn3)cc2F)s1]

Following GENERAL PROCEDURE 7 with 5-(3-(4-(3-((((4-((S)-2-((S)-2-amino-3-methylbutanamido)-5-ureidopentanamido)-2-((prop-2-yn-1-yl((prop-2-yn-1-yloxy)carbonyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid (20.8 mg, 0.017 mmol) and 1-azido-3,6,9,12,15,18,21,24-octaoxaheptacosan-27-oic acid (17.9 mg, 0.038 mmol, 2.2 equiv.), 5-(3-(4-(3-((((4-((S)-2-((S)-2-amino-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)((1-(26-carboxy-3,6,9,12,15,18,21,24-octaoxahexacosyl)-1H-1,2,3-triazol-4-yl)methyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid was obtained. LCMS: M/2+H=1068.2; Rt=0.99 min (2 min acidic method).

### Synthesis of 2-(3-(benzo[d]thiazol-2-ylamino)-4-methyl-6,7-dihydropyrido[2,3-c]pyridazin-8(5H)-yl)-5-(3-(4-(3-((((2-(((((1-(26-carboxy-3,6,9,12,15,18,21,24-octaoxahexacosyl)-1H-1,2,3-triazol-4-yl)methoxy)carbonyl)((1-(26-carboxy-3,6,9,12,15,18,21,24-octaoxahexacosyl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazole-4-carboxylic acid (L10C-P3)

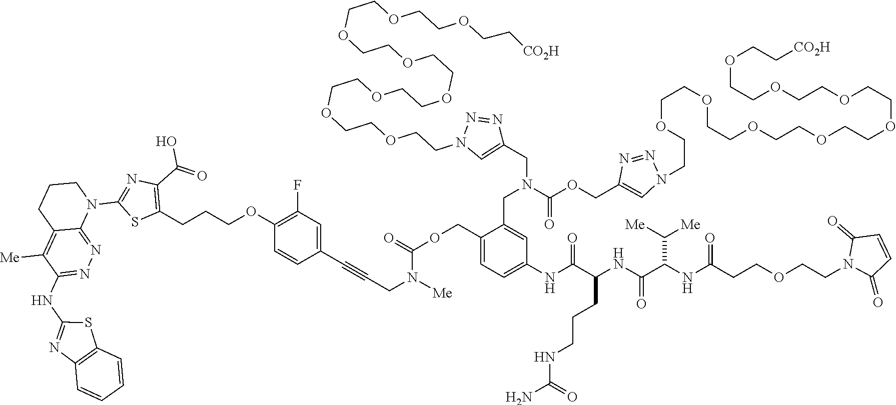 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3CN(Cc3cn(CCOCCOCCOCCOCCOCCOCCOCCOCCC(=O)O)nn3)C(=O)OCc3cn(CCOCCOCCOCCOCCOCCOCCOCCOCCC(=O)O)nn3)cc2F)s1]

Following GENERAL PROCEDURE 5 with 5-(3-(4-(3-((((4-((S)-2-((S)-2-amino-3-methylbutanamido)-5-ureidopentanamido)-2-(((((1-(26-carboxy-3,6,9,12,15,18,21,24-octaoxahexacosyl)-1H-1,2,3-triazol-4-yl)methoxy)carbonyl)((1-(26-carboxy-3,6,9,12,15,18,21,24-octaoxahexacosyl)-1H-1,2,3-triazol-4-yl)methyl)amino)methyl)benzyl)oxy)carbonyl)(methyl)amino)prop-1-yn-1-yl)-2-fluorophenoxy)propyl)-2-(3-(benzo[d]thiazol-2-ylamino)-4-methyl-6,7-dihydropyrido[2,3-c]pyridazin-8(5H)-yl)thiazole-4-carboxylic acid (36.3 mg, 0.017 mmol) and 2,5-dioxopyrrolidin-1-yl 3-(2-(2,5-dioxo-2,5-dihydro-1H-pyrrol-1-yl)ethoxy)propanoate (5.3 mg, 0.017 mmol, 1.0 equiv.), 2-(3-(benzo[d]thiazol-2-ylamino)-4-methyl-6,7-dihydropyrido[2,3-c]pyridazin-8(5H)-yl)-5-(3-(4-(3-((((2-(((((1-(26-carboxy-3,6,9,12,15,18,21,24-octaoxahexacosyl)-1H-1,2,3-triazol-4-yl)methoxy)carbonyl)((1-(26-carboxy-3,6,9,12,15,18,21,24-octaoxahexacosyl)-1H-1,2,3-triazol-4-yl)methyl)amino)methyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazole-4-carboxylic acid was obtained. HRMS: M+H=2327.9800; Rt=2.45 min (5 min acidic method).

### Synthesis of 5-((6-(benzo[d]thiazol-2-ylamino)-5-methylpyridazin-3-yl) (4-carboxy-5-(3-(4-(3-((((4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazol-2-yl)amino)-2-hydroxy-N,N-dimethyl-N-(3-sulfopropyl)pentan-1-aminium (L9C-P4)

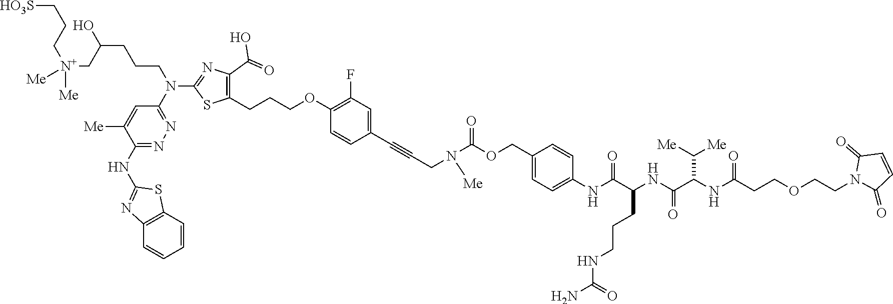 [SMILES: Cc1cc(N(CCCC(O)C[N+](C)(C)CCCS(=O)(=O)O)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1]

Following GENERAL PROCEDURE 10 with 4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl(4-nitrophenyl) carbonate (20.0 mg, 0.027 mmol) and 5-((6-(benzo[d]thiazol-2-ylamino)-5-methylpyridazin-3-yl) (4-carboxy-5-(3-(2-fluoro-4-(3-(methylamino)prop-1-yn-1-yl) phenoxy)propyl)thiazol-2-yl)amino)-2-hydroxy-N,N-dimethyl-N-(3-sulfopropyl)pentan-1-aminium (23.1 mg, 0.027 mmol, 1.0 equiv.), 5-((6-(benzo[d]thiazol-2-ylamino)-5-methylpyridazin-3-yl) (4-carboxy-5-(3-(4-(3-((((4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazol-2-yl)amino)-2-hydroxy-N,N-dimethyl-N-(3-sulfopropyl)pentan-1-aminium was obtained. HRMS: M+H=1455.5300; Rt=2.31 min (5 min acidic method).

### Synthesis of 2-((6-(benzo[d]thiazol-2-ylamino)-5-methylpyridazin-3-yl) (4-hydroxy-5-(trimethylammonio) pentyl)amino)-5-(3-(4-(3-((((4-((S)-2-((S)-2-((tert-butoxycarbonyl)amino)-3-methylbutanamido)-5-ureidopentanamido)benzyl)oxy)carbonyl)(methyl)amino)prop-1-yn-1-yl)-2-fluorophenoxy)propyl)thiazole-4-carboxylate

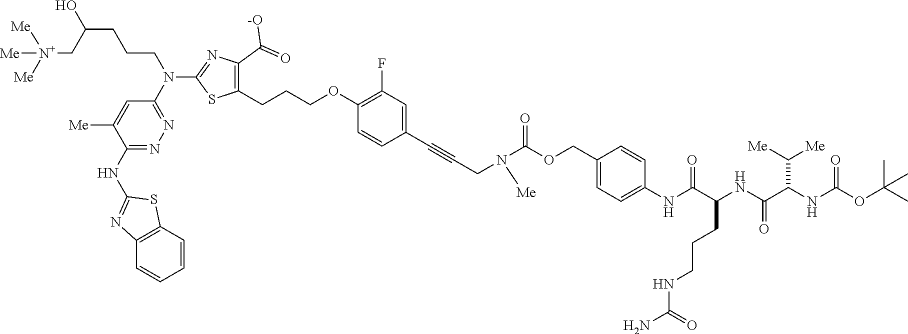

Following GENERAL PROCEDURE 10 with 2-((6-(benzo[d]thiazol-2-ylamino)-5-methylpyridazin-3-yl) (4-hydroxy-5-(trimethylammonio) pentyl)amino)-5-(3-(2-fluoro-4-(3-(methylamino)prop-1-yn-1-yl) phenoxy)propyl)thiazole-4-carboxylate (40.0 mg, 0.054 mmol) and tert-butyl((S)-3-methyl-1-(((S)-1-((4-((((4-nitrophenoxy)carbonyl)oxy)methyl)phenyl)amino)-1-oxo-5-ureidopentan-2-yl)amino)-1-oxobutan-2-yl) carbamate (34.5 mg, 0.054 mmol, 1.0 equiv.), 2-((6-(benzo[d]thiazol-2-ylamino)-5-methylpyridazin-3-yl) (4-hydroxy-5-(trimethylammonio) pentyl)amino)-5-(3-(4-(3-((((4-((S)-2-((S)-2-((tert-butoxycarbonyl)amino)-3-methylbutanamido)-5-ureidopentanamido)benzyl)oxy)carbonyl)(methyl)amino)prop-1-yn-1-yl)-2-fluorophenoxy)propyl)thiazole-4-carboxylate was obtained. LCMS: M+H=1253.8; Rt=1.11 min (2 min acidic method).

### Synthesis of 5-(3-(4-(3-((((4-((S)-2-((S)-2-amino-3-methylbutanamido)-5-ureidopentanamido)benzyl)oxy)carbonyl)(methyl)amino)prop-1-yn-1-yl)-2-fluorophenoxy)propyl)-2-((6-(benzo[d]thiazol-2-ylamino)-5-methylpyridazin-3-yl) (4-hydroxy-5-(trimethylammonio) pentyl)amino)thiazole-4-carboxylate

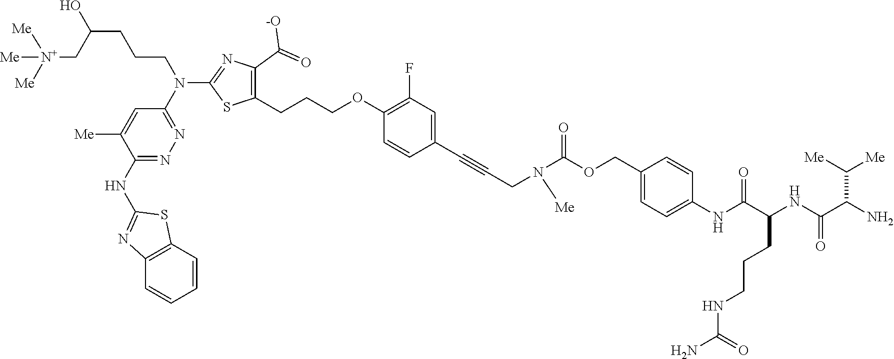

Following GENERAL PROCEDURE 4 with 2-((6-(benzo[d]thiazol-2-ylamino)-5-methylpyridazin-3-yl) (4-hydroxy-5-(trimethylammonio) pentyl)amino)-5-(3-(4-(3-((((4-((S)-2-((S)-2-((tert-butoxycarbonyl)amino)-3-methylbutanamido)-5-ureidopentanamido)benzyl)oxy)carbonyl)(methyl)amino)prop-1-yn-1-yl)-2-fluorophenoxy)propyl)thiazole-4-carboxylate (64.8 mg, 0.052 mmol), 5-(3-(4-(3-((((4-((S)-2-((S)-2-amino-3-methylbutanamido)-5-ureidopentanamido)benzyl)oxy)carbonyl)(methyl)amino)prop-1-yn-1-yl)-2-fluorophenoxy)propyl)-2-((6-(benzo[d]thiazol-2-ylamino)-5-methylpyridazin-3-yl) (4-hydroxy-5-(trimethylammonio) pentyl)amino)thiazole-4-carboxylate was obtained. LCMS: M/2+H=576.6; Rt=0.99 min (2 min acidic method).

### Synthesis of 5-((6-(benzo[d]thiazol-2-ylamino)-5-methylpyridazin-3-yl) (4-carboxy-5-(3-(4-(3-((((4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazol-2-yl)amino)-2-hydroxy-N,N,N-trimethylpentan-1-aminium (L9C-P5)

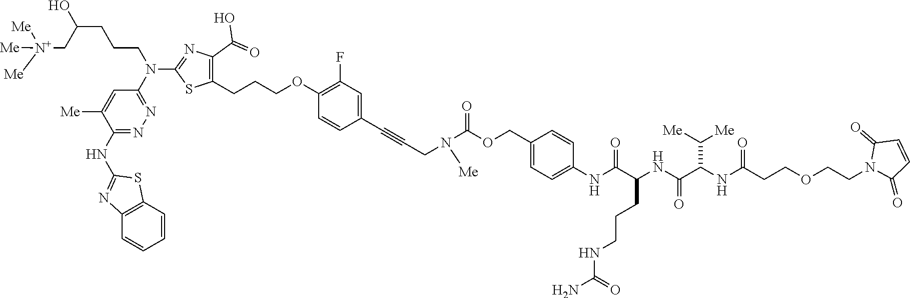 [SMILES: Cc1cc(N(CCCC(O)C[N+](C)(C)C)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1]

Following GENERAL PROCEDURE 5 with 5-(3-(4-(3-((((4-((S)-2-((S)-2-amino-3-methylbutanamido)-5-ureidopentanamido)benzyl)oxy)carbonyl)(methyl)amino)prop-1-yn-1-yl)-2-fluorophenoxy)propyl)-2-((6-(benzo[d]thiazol-2-ylamino)-5-methylpyridazin-3-yl) (4-hydroxy-5-(trimethylammonio) pentyl)amino)thiazole-4-carboxylate (59.0 mg, 0.051 mmol) and 2,5-dioxopyrrolidin-1-yl 3-(2-(2,5-dioxo-2,5-dihydro-1H-pyrrol-1-yl)ethoxy)propanoate (19.1 mg, 0.061 mmol, 1.2 equiv.), 5-((6-(benzo[d]thiazol-2-ylamino)-5-methylpyridazin-3-yl) (4-carboxy-5-(3-(4-(3-((((4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(methyl)amino)prop-1-yn-1-yl)-2-fluorophenoxy)propyl)thiazol-2-yl)amino)-2-hydroxy-N,N,N-trimethylpentan-1-aminium was obtained. HRMS: M+H=1347.5300; Rt=2.23 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)pyrrolidin-1-ium

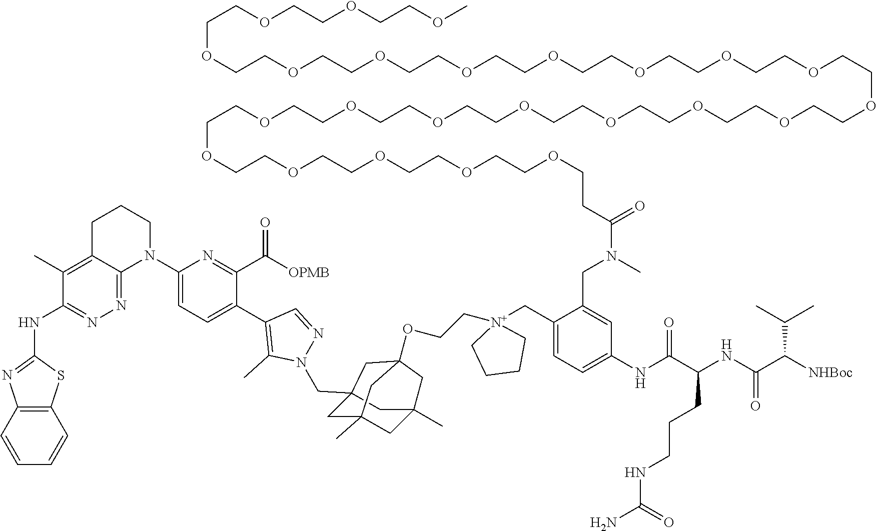

Following GENERAL PROCEDURE 3 with 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-((methylamino)methyl)benzyl)pyrrolidin-1-ium (40 mg, 0.028 mmol) and 2,5-dioxopyrrolidin-1-yl 2,5,8,11,14,17,20,23,26,29,32,35,38,41,44,47,50,53,56,59,62,65,68,71-tetracosaoxatetraheptacontan-74-oate (51.5 mg, 0.042 mmol, 1.5 equiv.), 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)pyrrolidin-1-ium was obtained. HRMS: M+=2511.4099; Rt=2.44 min (5 min acidic method).

### Synthesis of 1-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)pyrrolidin-1-ium

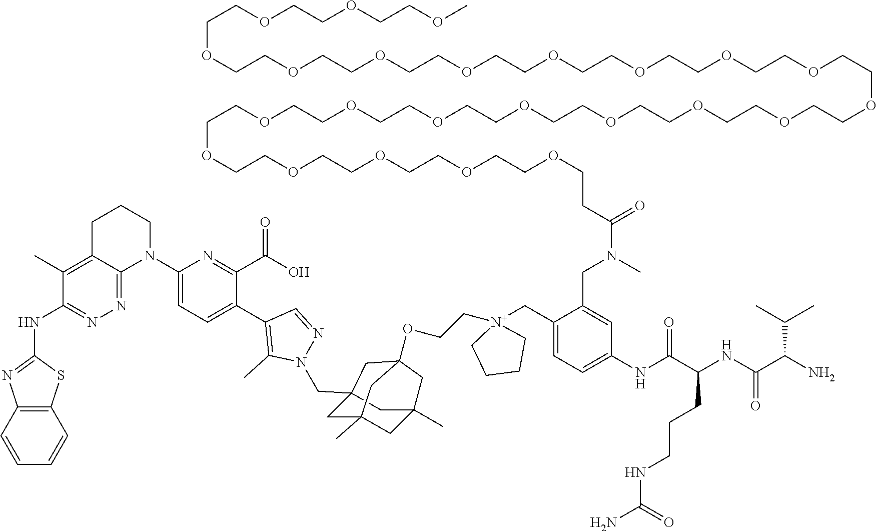 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](N)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1]

Following GENERAL PROCEDURE 4 with 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)pyrrolidin-1-ium (50 mg, 0.0199 mmol), 1-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)pyrrolidin-1-ium was obtained. HRMS: M+=2291.3101; Rt=1.93 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)-1-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)pyrrolidin-1-ium (L30A-P21)

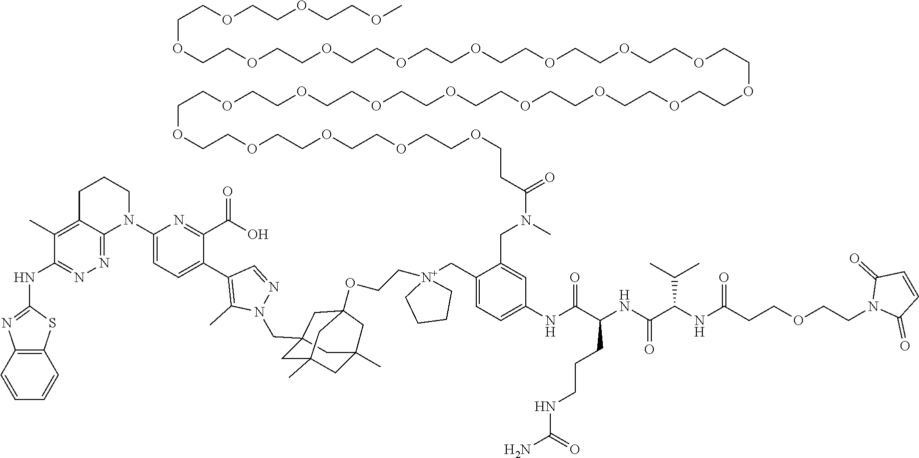 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1]

Following GENERAL PROCEDURE 5 with 1-(4-((S)-2-((S)-2-amino-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)pyrrolidin-1-ium (37 mg, 0.015 mmol) and 2,5-dioxopyrrolidin-1-yl 3-(2-(2,5-dioxo-2,5-dihydro-1H-pyrrol-1-yl)ethoxy)propanoate (11.4 mg, 0.0367 mmol, 2.5 equiv.), 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)-1-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-(75-methyl-74-oxo-2,5,8,11,14,17,20,23,26,29,32,35,38,41,44,47,50,53,56,59,62,65,68,71-tetracosaoxa-75-azahexaheptacontan-76-yl)benzyl)pyrrolidin-1-ium was obtained. HRMS: M+=2486.3301; Rt=2.14 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(2,5,8,11,14,17,20,23,26,29,32,35,38,44,44-pentadecamethyl-3,6,9,12,15,18,21,24,27,30,33,36,39,42-tetradecaoxo-43-oxa-2,5,8,11,14,17,20,23,26,29,32,35,38-tridecaazapentatetracontyl)benzyl)pyrrolidin-1-ium

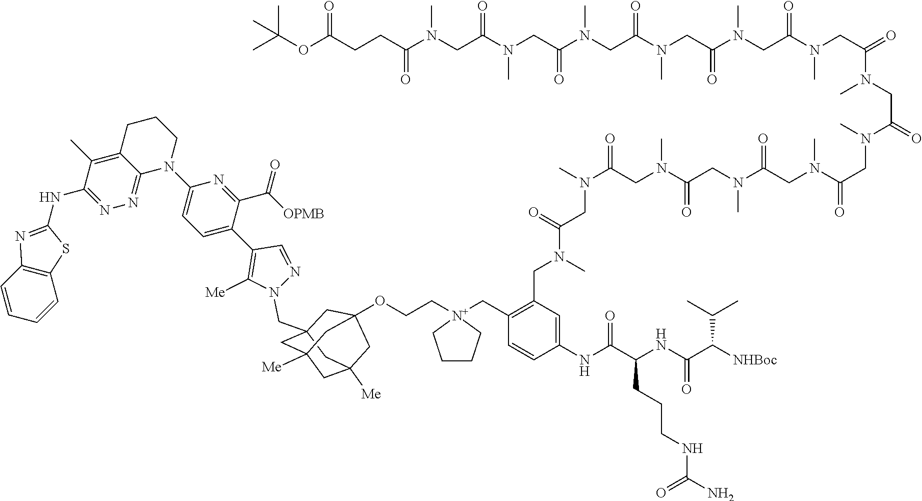

Following GENERAL PROCEDURE 3 with 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-((methylamino)methyl)benzyl)pyrrolidin-1-ium (35 mg, 0.021 mmol) and 3,6,9,12,15,18,21,24,27,30,33,36,42,42-tetradecamethyl-4,7,10,13,16,19,22,25,28,31,34,37,40-tridecaoxo-41-oxa-3,6,9,12,15,18,21,24,27,30,33,36-dodecaazatritetracontanoic acid (21.9 mg, 0.021 mmol, 1.0 equiv.), 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(2,5,8,11,14,17,20,23,26,29,32,35,38,44,44-pentadecamethyl-3,6,9,12,15,18,21,24,27,30,33,36,39,42-tetradecaoxo-43-oxa-2,5,8,11,14,17,20,23,26,29,32,35,38-tridecaazapentatetracontyl)benzyl)pyrrolidin-1-ium was obtained. HRMS: [(M+)+H+]⁺²/2=1211.6500; Rt-2.31 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)-1-(2-(41-carboxy-2,5,8,11,14,17,20,23,26,29,32,35,38-tridecamethyl-3,6,9,12,15,18,21,24,27,30,33,36,39-tridecaoxo-2,5,8,11,14,17,20,23,26,29,32,35,38-tridecaazahentetracontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)pyrrolidin-1-ium (L35A-P21)

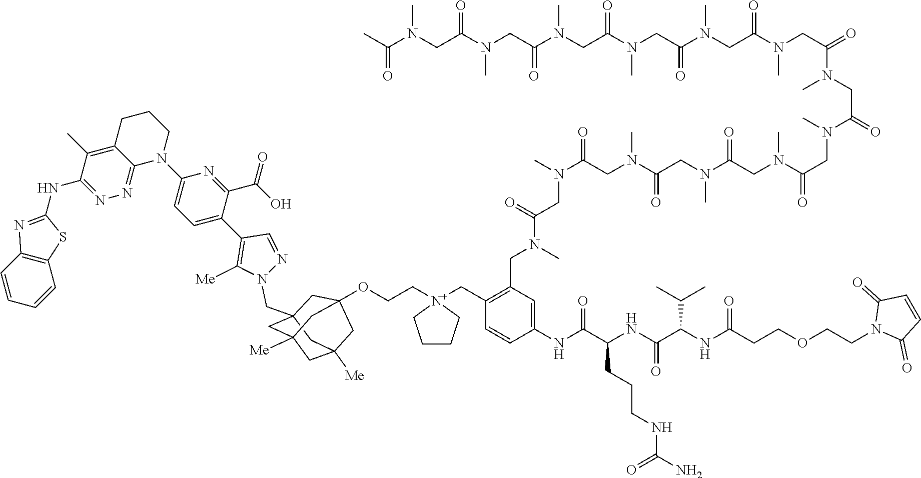 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1]

Following GENERAL PROCEDURE 4 with 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(2,5,8,11,14,17,20,23,26,29,32,35,38,44,44-pentadecamethyl-3,6,9,12,15,18,21,24,27,30,33,36,39,42-tetradecaoxo-43-oxa-2,5,8,11,14,17,20,23,26,29,32,35,38-tridecaazapentatetracontyl)benzyl)pyrrolidin-1-ium (24 mg, 0.0095 mmol) and then taking the crude product on and following GENERAL PROCEDURE 5 with 2,5-dioxopyrrolidin-1-yl 3-(2-(2,5-dioxo-2,5-dihydro-1H-pyrrol-1-yl)ethoxy)propanoate (5.9 mg, 0.019 mmol, 2 equiv.), 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)-1-(2-(41-carboxy-2,5,8,11,14,17,20,23,26,29,32,35,38-tridecamethyl-3,6,9,12,15,18,21,24,27,30,33,36,39-tridecaoxo-2,5,8,11,14,17,20,23,26,29,32,35,38-tridecaazahentetracontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)pyrrolidin-1-ium was obtained. HRMS: M+=2340.1699; Rt=1.87 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(2,5,8,11,14,17,20,23,26,29,32,35,38,41,44,47,50,53,56,62,62-henicosamethyl-3,6,9,12,15,18,21,24,27,30,33,36,39,42,45,48,51,54,57,60-icosaoxo-61-oxa-2,5,8,11,14,17,20,23,26,29,32,35,38,41,44,47,50,53,56-nonadecaazatrihexacontyl)benzyl)pyrrolidin-1-ium

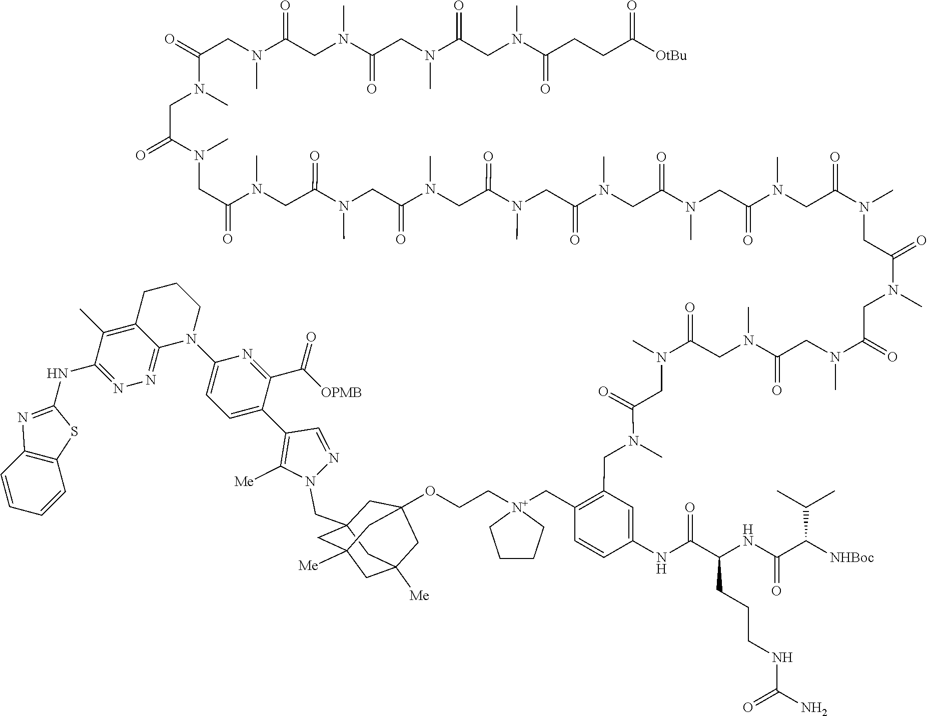

Following GENERAL PROCEDURE 3 with 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-((methylamino)methyl)benzyl)pyrrolidin-1-ium (47 mg, 0.0286 mmol) and 3,6,9,12,15,18,21,24,27,30,33,36,39,42,45,48,51,54,60,60-icosamethyl-4,7,10,13,16,19,22,25,28,31,34,37,40,43,46,49,52,55,58-nonadecaoxo-59-oxa-3,6,9,12,15,18,21,24,27,30,33,36,39,42,45,48,51,54-octadecaazahenhexacontanoic acid (41.6 mg, 0.0286 mmol, 1.0 equiv.), 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(2,5,8,11,14,17,20,23,26,29,32,35,38,41,44,47,50,53,56,62,62-henicosamethyl-3,6,9,12,15,18,21,24,27,30,33,36,39,42,45,48,51,54,57,60-icosaoxo-61-oxa-2,5,8,11,14,17,20,23,26,29,32,35,38,41,44,47,50,53,56-nonadecaazatrihexacontyl)benzyl)pyrrolidin-1-ium was obtained. HRMS: M+=2487.5400; Rt-2.26 min (5 min acidic method).

### Synthesis of 1-(4-((S)-2-((S)-2-amino-3-methylbutanamido)-5-ureidopentanamido)-2-(59-carboxy-2,5,8,11,14,17,20,23,26,29,32,35,38,41,44,47,50,53,56-nonadecamethyl-3,6,9,12,15,18,21,24,27,30,33,36,39,42,45,48,51,54,57-nonadecaoxo-2,5,8,11,14,17,20,23,26,29,32,35,38,41,44,47,50,53,56-nonadecaazanonapentacontyl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)pyrrolidin-1-ium

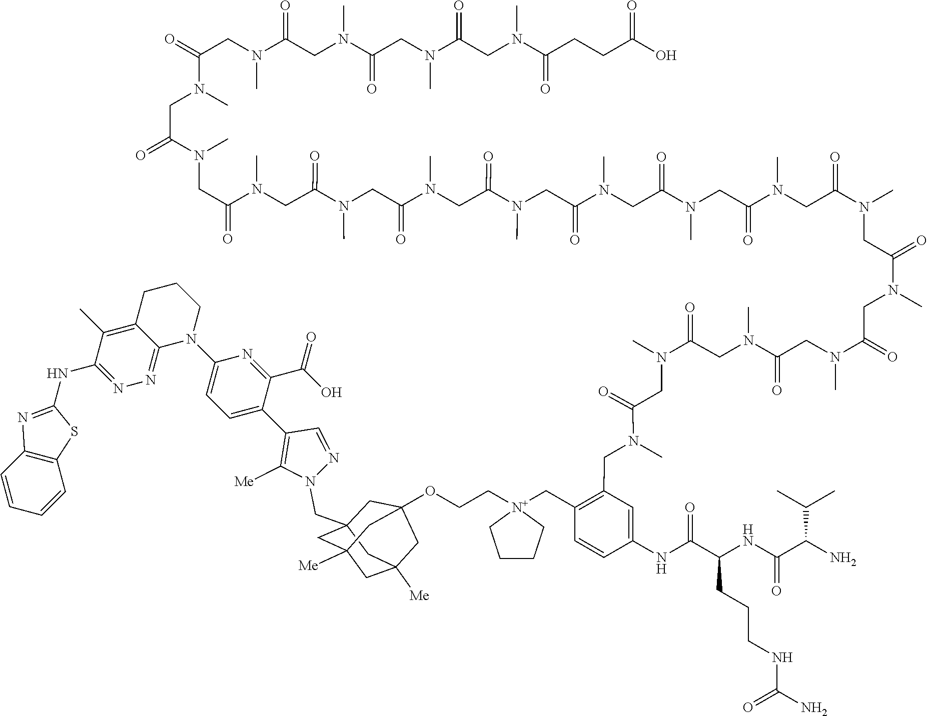 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](N)C(C)C)cc6CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)CCCC3)(C5)C4)c2C)c(C(=O)O)n1]

Following GENERAL PROCEDURE 4 with 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(2,5,8,11,14,17,20,23,26,29,32,35,38,41,44,47,50,53,56,62,62-henicosamethyl-3,6,9,12,15,18,21,24,27,30,33,36,39,42,45,48,51,54,57,60-icosaoxo-61-oxa-2,5,8,11,14,17,20,23,26,29,32,35,38,41,44,47,50,53,56-nonadecaazatrihexacontyl)benzyl)pyrrolidin-1-ium (46 mg, 0.0155 mmol), 1-(4-((S)-2-((S)-2-amino-3-methylbutanamido)-5-ureidopentanamido)-2-(59-carboxy-2,5,8,11,14,17,20,23,26,29,32,35,38,41,44,47,50,53,56-nonadecamethyl-3,6,9,12,15,18,21,24,27,30,33,36,39,42,45,48,51,54,57-nonadecaoxo-2,5,8,11,14,17,20,23,26,29,32,35,38,41,44,47,50,53,56-nonadecaazanonapentacontyl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)pyrrolidin-1-ium was obtained. HRMS: M+=2571.3401; Rt=1.60 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)-1-(2-(59-carboxy-2,5,8,11,14,17,20,23,26,29,32,35,38,41,44,47,50,53,56-nonadecamethyl-3,6,9,12,15,18,21,24,27,30,33,36,39,42,45,48,51,54,57-nonadecaoxo-2,5,8,11,14,17,20,23,26,29,32,35,38,41,44,47,50,53,56-nonadecaazanonapentacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)pyrrolidin-1-ium (L36A-P21)

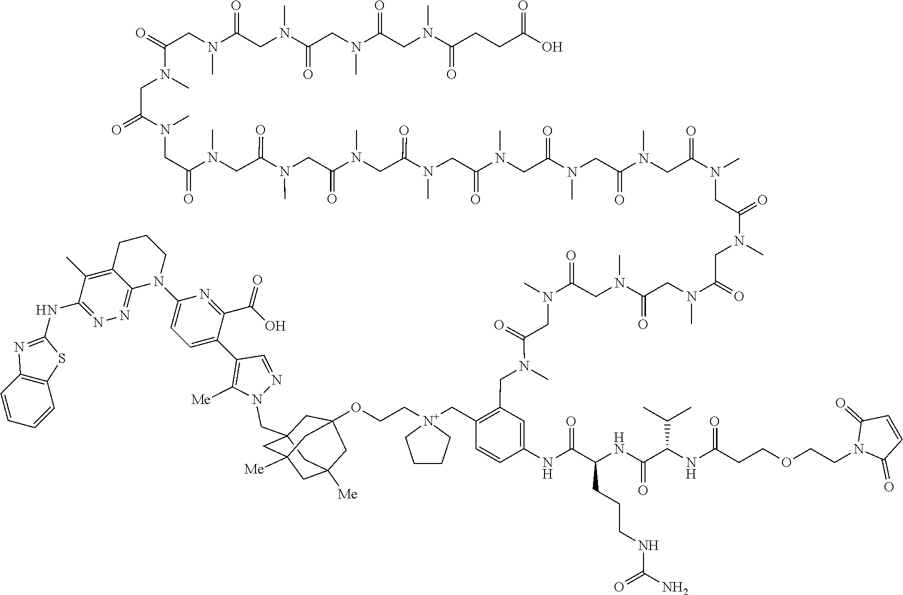 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)CCCC3)(C5)C4)c2C)c(C(=O)O)n1]

Following GENERAL PROCEDURE 5 with 1-(4-((S)-2-((S)-2-amino-3-methylbutanamido)-5-ureidopentanamido)-2-(59-carboxy-2,5,8,11,14,17,20,23,26,29,32,35,38,41,44,47,50,53,56-nonadecamethyl-3,6,9,12,15,18,21,24,27,30,33,36,39,42,45,48,51,54,57-nonadecaoxo-2,5,8,11,14,17,20,23,26,29,32,35,38,41,44,47,50,53,56-nonadecaazanonapentacontyl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)pyrrolidin-1-ium (17.0 mg, 0.0055 mmol) and 2,5-dioxopyrrolidin-1-yl 3-(2-(2,5-dioxo-2,5-dihydro-1H-pyrrol-1-yl)ethoxy)propanoate (2.4 mg, 0.0076 mmol, 1.4 equiv.), 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)-1-(2-(59-carboxy-2,5,8,11,14,17,20,23,26,29,32,35,38,41,44,47,50,53,56-nonadecamethyl-3,6,9,12,15,18,21,24,27,30,33,36,39,42,45,48,51,54,57-nonadecaoxo-2,5,8,11,14,17,20,23,26,29,32,35,38,41,44,47,50,53,56-nonadecaazanonapentacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)pyrrolidin-1-ium was obtained. HRMS: M+=2766.3899; Rt=1.82 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(2,5,8,11,14,17,20,23,26,29,32,35,38,41,44,47,50,53,56,59,62,65,68,71,74,80,80-heptacosamethyl-3,6,9,12,15,18,21,24,27,30,33,36,39,42,45,48,51,54,57,60,63,66,69,72,75,78-hexacosaoxo-79-oxa-2,5,8,11,14,17,20,23,26,29,32,35,38,41,44,47,50,53,56,59,62,65,68,71,74-pentacosaazahenoctacontyl)benzyl)pyrrolidin-1-ium

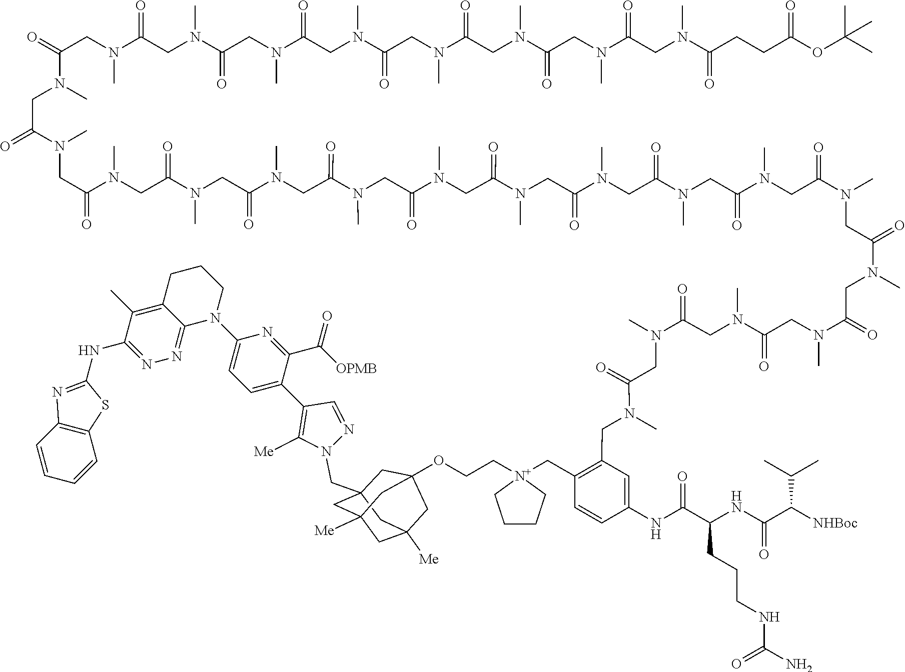

Following GENERAL PROCEDURE 3 with 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-((methylamino)methyl)benzyl)pyrrolidin-1-ium (40 mg, 0.028 mmol) and 3,6,9,12,15,18,21,24,27,30,33,36,39,42,45,48,51,54,57,60,63,66,69,72,78,78-hexacosamethyl-4,7,10,13,16,19,22,25,28,31,34,37,40,43,46,49,52,55,58,61,64,67,70,73,76-pentacosaoxo-77-oxa-3,6,9,12,15,18,21,24,27,30,33,36,39,42,45,48,51,54,57,60,63,66,69,72-tetracosaazanonaheptacontanoic acid (58.5 mg, 0.031 mmol, 1.1 equiv.), 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(2,5,8,11,14,17,20,23,26,29,32,35,38,41,44,47,50,53,56,59,62,65,68,71,74,80,80-heptacosamethyl-3,6,9,12,15,18,21,24,27,30,33,36,39,42,45,48,51,54,57,60,63,66,69,72,75,78-hexacosaoxo-79-oxa-2,5,8,11,14,17,20,23,26,29,32,35,38,41,44,47,50,53,56,59,62,65,68,71,74-pentacosaazahenoctacontyl)benzyl)pyrrolidin-1-ium was obtained. HRMS: M+=3273.7500; Rt=2.24 min (5 min acidic method).

### Synthesis of 1-(4-((S)-2-((S)-2-amino-3-methylbutanamido)-5-ureidopentanamido)-2-(77-carboxy-2,5,8,11,14,17,20,23,26,29,32,35,38,41,44,47,50,53,56,59,62,65,68,71,74-pentacosamethyl-3,6,9,12,15,18,21,24,27,30,33,36,39,42,45,48,51,54,57,60,63,66,69,72,75-pentacosaoxo-2,5,8,11,14,17,20,23,26,29,32,35,38,41,44,47,50,53,56,59,62,65,68,71,74-pentacosaazaheptaheptacontyl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)pyrrolidin-1-ium

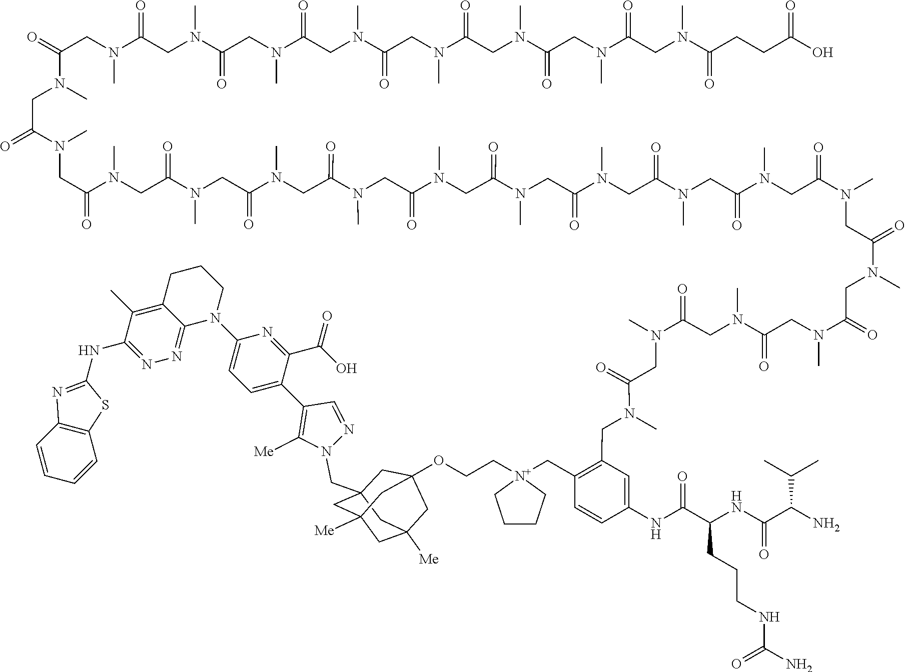 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](N)C(C)C)cc6CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)CCCC3)(C5)C4)c2C)c(C(=O)O)n1]

Following GENERAL PROCEDURE 4 with 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(2,5,8,11,14,17,20,23,26,29,32,35,38,41,44,47,50,53,56,59,62,65,68,71,74,80,80-heptacosamethyl-3,6,9,12,15,18,21,24,27,30,33,36,39,42,45,48,51,54,57,60,63,66,69,72,75,78-hexacosaoxo-79-oxa-2,5,8,11,14,17,20,23,26,29,32,35,38,41,44,47,50,53,56,59,62,65,68,71,74-pentacosaazahenoctacontyl)benzyl)pyrrolidin-1-ium (20 mg, 0.0063 mmol), 1-(4-((S)-2-((S)-2-amino-3-methylbutanamido)-5-ureidopentanamido)-2-(77-carboxy-2,5,8,11,14,17,20,23,26,29,32,35,38,41,44,47,50,53,56,59,62,65,68,71,74-pentacosamethyl-3,6,9,12,15,18,21,24,27,30,33,36,39,42,45,48,51,54,57,60,63,66,69,72,75-pentacosaoxo-2,5,8,11,14,17,20,23,26,29,32,35,38,41,44,47,50,53,56,59,62,65,68,71,74-pentacosaazaheptaheptacontyl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)pyrrolidin-1-ium was obtained. HRMS: M+=2997.6001; Rt=1.65 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)-1-(2-(77-carboxy-2,5,8,11,14,17,20,23,26,29,32,35,38,41,44,47,50,53,56,59,62,65,68,71,74-pentacosamethyl-3,6,9,12,15,18,21,24,27,30,33,36,39,42,45,48,51,54,57,60,63,66,69,72,75-pentacosaoxo-2,5,8,11,14,17,20,23,26,29,32,35,38,41,44,47,50,53,56,59,62,65,68,71,74-pentacosaazaheptaheptacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)pyrrolidin-1-ium (L37A-P21)

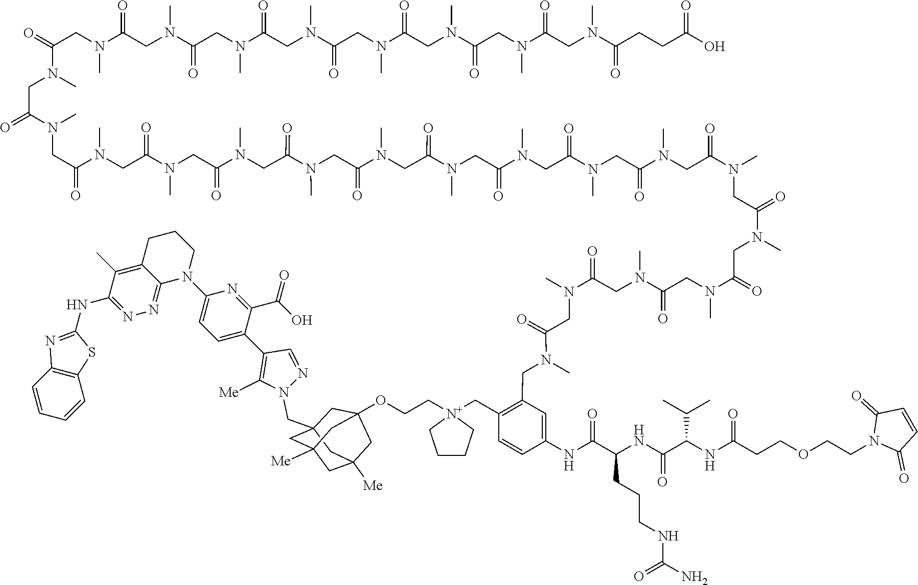 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)CCCC3)(C5)C4)c2C)c(C(=O)O)n1]

Following GENERAL PROCEDURE 5 with 1-(4-((S)-2-((S)-2-amino-3-methylbutanamido)-5-ureidopentanamido)-2-(77-carboxy-2,5,8,11,14,17,20,23,26,29,32,35,38,41,44,47,50,53,56,59,62,65,68,71,74-pentacosamethyl-3,6,9,12,15,18,21,24,27,30,33,36,39,42,45,48,51,54,57,60,63,66,69,72,75-pentacosaoxo-2,5,8,11,14,17,20,23,26,29,32,35,38,41,44,47,50,53,56,59,62,65,68,71,74-pentacosaazaheptaheptacontyl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)pyrrolidin-1-ium (35 mg, 0.011 mmol) and 2,5-dioxopyrrolidin-1-yl 3-(2-(2,5-dioxo-2,5-dihydro-1H-pyrrol-1-yl)ethoxy)propanoate (4.7 mg, 0.015 mmol, 1.4 equiv.), 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)-1-(2-(77-carboxy-2,5,8,11,14,17,20,23,26,29,32,35,38,41,44,47,50,53,56,59,62,65,68,71,74-pentacosamethyl-3,6,9,12,15,18,21,24,27,30,33,36,39,42,45,48,51,54,57,60,63,66,69,72,75-pentacosaoxo-2,5,8,11,14,17,20,23,26,29,32,35,38,41,44,47,50,53,56,59,62,65,68,71,74-pentacosaazaheptaheptacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)pyrrolidin-1-ium was obtained. HRMS: M+=3192.6399; Rt=1.86 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(2,5,8,11,14,17,20,23,26,29,32,35,38-tridecamethyl-3,6,9,12,15,18,21,24,27,30,33,36,39-tridecaoxo-2,5,8,11,14,17,20,23,26,29,32,35,38-tridecaazatetracontyl)benzyl)pyrrolidin-1-ium

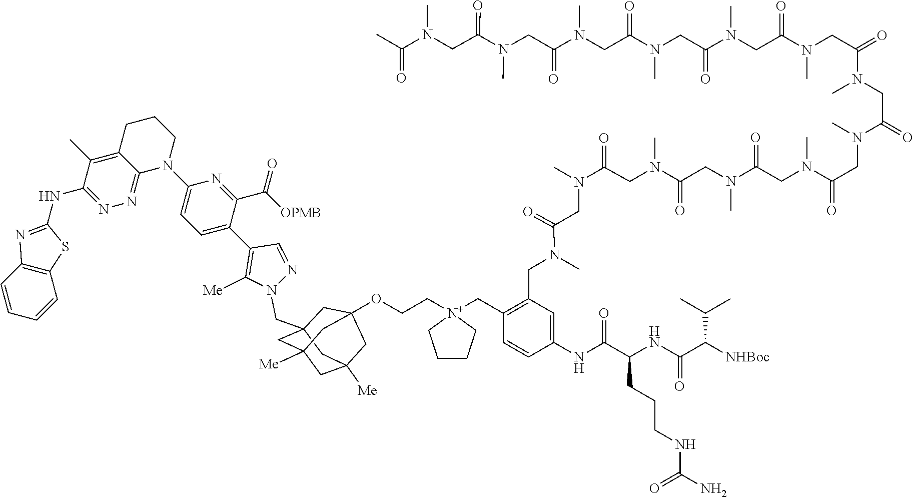

Following GENERAL PROCEDURE 3 with 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-((methylamino)methyl)benzyl)pyrrolidin-1-ium (70 mg, 0.043 mmol) and 3,6,9,12,15,18,21,24,27,30,33,36-dodecamethyl-4,7,10,13,16,19,22,25,28,31,34,37-dodecaoxo-3,6,9,12,15,18,21,24,27,30,33,36-dodecaazaoctatriacontanoic acid (38.9 mg, 0.043 mmol, 1.0 equiv.), 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(2,5,8,11,14,17,20,23,26,29,32,35,38-tridecamethyl-3,6,9,12,15,18,21,24,27,30,33,36,39-tridecaoxo-2,5,8,11,14,17,20,23,26,29,32,35,38-tridecaazatetracontyl)benzyl)pyrrolidin-1-ium was obtained. HRMS: M+=2307.2300; Rt=2.20 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)-1-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-(2,5,8,11,14,17,20,23,26,29,32,35,38-tridecamethyl-3,6,9,12,15,18,21,24,27,30,33,36,39-tridecaoxo-2,5,8,11,14,17,20,23,26,29,32,35,38-tridecaazatetracontyl)benzyl)pyrrolidin-1-ium (L38A-P21)

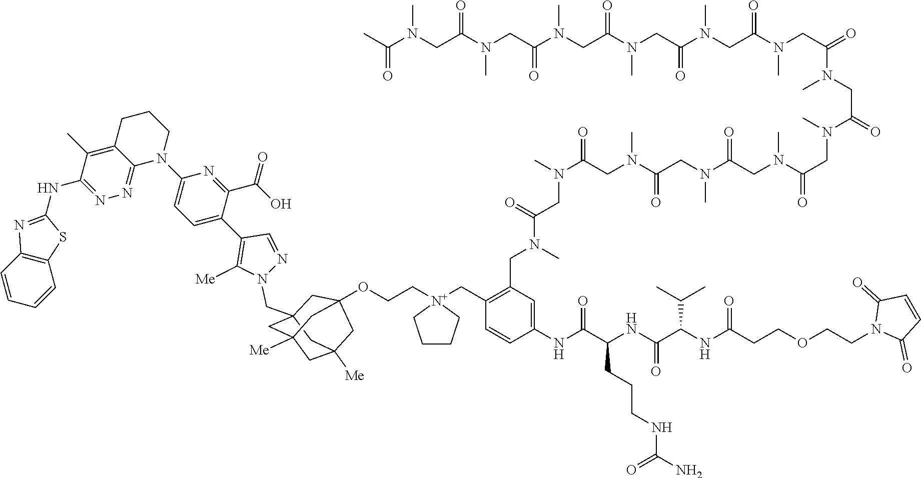 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1]

Following GENERAL PROCEDURE 4 with 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(2,5,8,11,14,17,20,23,26,29,32,35,38-tridecamethyl-3,6,9,12,15,18,21,24,27,30,33,36,39-tridecaoxo-2,5,8,11,14,17,20,23,26,29,32,35,38-tridecaazatetracontyl)benzyl)pyrrolidin-1-ium (67 mg, 0.029 mmol) and then taking the crude reaction product and following GENERAL PROCEDURE 5 with 2,5-dioxopyrrolidin-1-yl 3-(2-(2,5-dioxo-2,5-dihydro-1H-pyrrol-1-yl)ethoxy)propanoate (13.5 mg, 0.044 mmol, 1.5 equiv.), 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)-1-(4-((S)-2-((S)-2-(3-(2-(2,5-dioxo-2,5-dihydro-1H-pyrrol-1-yl)ethoxy)propanamido)-3-methylbutanamido)-5-ureidopentanamido)-2-(2,5,8,11,14,17,20,23,26,29,32,35,38-tridecamethyl-3,6,9,12,15,18,21,24,27,30,33,36,39-tridecaoxo-2,5,8,11,14,17,20,23,26,29,32,35,38-tridecaazatetracontyl)benzyl)pyrrolidin-1-iumwas obtained. HRMS: M+=2282.2500; Rt=1.89 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)pyrrolidin-1-ium

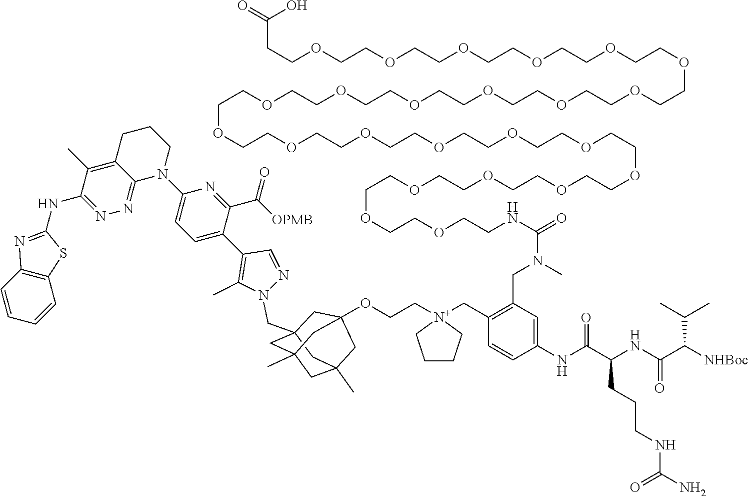

A mixture of 1-amino-3,6,9,12,15,18,21,24,27,30,33,36,39,42,45,48,51,54,57,60,63,66,69,72-tetracosaoxapentaheptacontan-75-oic acid (67 mg, 0.059 mmol, 1.28 equiv.), bis(4-nitrophenyl) carbonate (17 mg, 0.057 mmol, 1.25 equiv.), and DIPEA (48 μL, 0.28 mmol, 6.0 equiv.)) in DMF (1 mL) was stirred at RT for 1 h at which time 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-((methylamino)methyl)benzyl)pyrrolidin-1-ium (65 mg, 0.046 mmol, 1.0 equiv.) and additional DIEA (80 μL, 0.46 mmol, 10 equiv.) were added. After stirring for 1 hour the solution was diluted with DMSO (2.5 mL) and purified by RP-HPLC. After lyophilization, 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(78-carboxy-2-methyl-3-oxo-7,10, 13, 16, 19,22,25,28,31,34,37,40,43,46,49,52,55,58,61,64,67,70,73,76-tetracosaoxa-2,4-diazaoctaheptacontyl)benzyl)pyrrolidin-1-ium was obtained. HRMS: M+=2584.4399; Rt-2.39 min (5 min acidic method).

### Synthesis of 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)pyrrolidin-1-ium

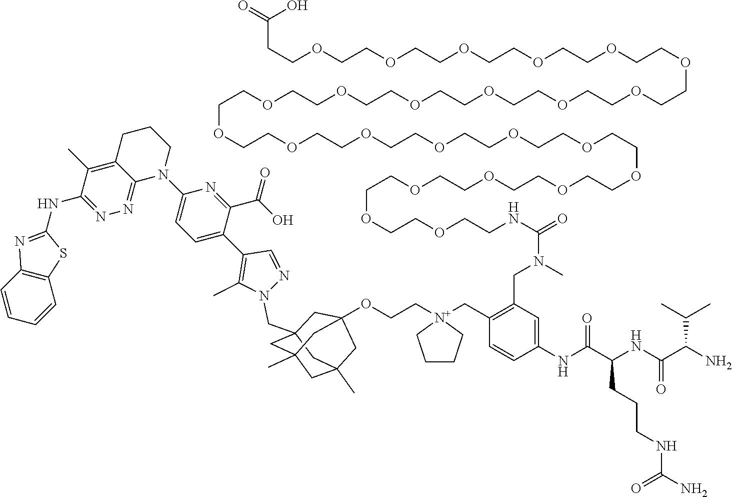 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](N)C(C)C)cc6CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CCCC3)(C5)C4)c2C)c(C(=O)O)n1]

Following GENERAL PROCEDURE 4 with 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)pyrrolidin-1-ium (58 mg, 0.021 mmol), 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)pyrrolidin-1-ium was obtained. HRMS: [(M+)+H+)]⁺²/2=1183.1700; Rt=1.88 min (5 min acidic method).

### Synthesis of 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)-1-(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)pyrrolidin-1-ium (L42A-P21)

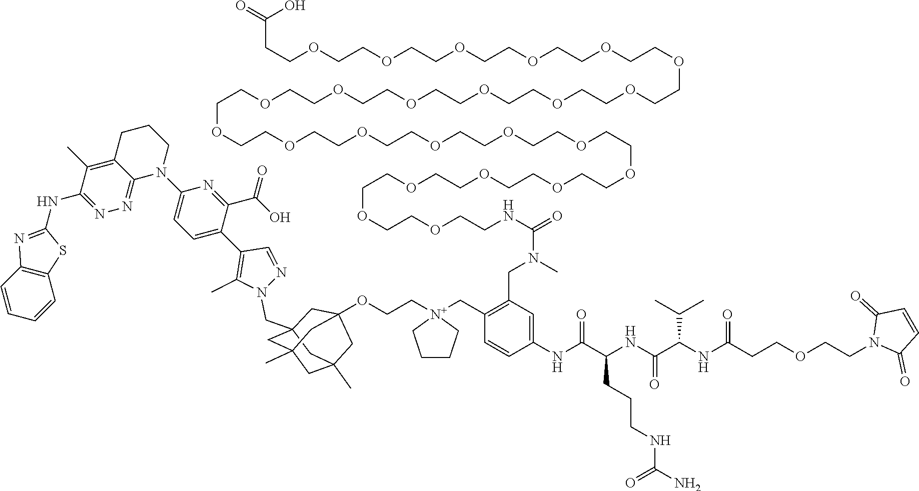 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CCCC3)(C5)C4)c2C)c(C(=O)O)n1]

Following GENERAL PROCEDURE 5 with 1-(4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)pyrrolidin-1-ium (61 mg, 0.024 mmol) and 2,5-dioxopyrrolidin-1-yl 3-(2-(2,5-dioxo-2,5-dihydro-1H-pyrrol-1-yl)ethoxy)propanoate (10.2 mg, 0.033 mmol, 1.4 equiv.), 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-carboxypyridin-3-yl)-5-methyl-1H-pyrazol-1-yl)methyl)-5,7-dimethyladamantan-1-yl)oxy)ethyl)-1-(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)pyrrolidin-1-ium was obtained. HRMS: M+=2559.3701; Rt=2.07 min (5 min acidic method).

### Synthesis of 1-(2-((((2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-5-((S)-2-((S)-2-((tert-butoxycarbonyl)amino)-3-methylbutanamido)-5-ureidopentanamido)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid

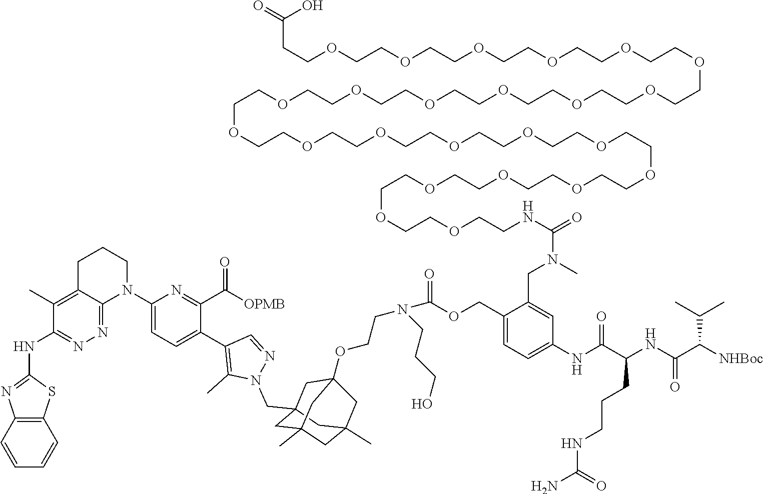

A mixture of 1-amino-3,6,9,12,15,18,21,24,27,30,33,36,39,42,45,48,51,54,57,60,63,66,69,72-tetracosaoxapentaheptacontan-75-oic acid (45.9 mg, 0.040 mmol, 1.3 equiv.), bis(4-nitrophenyl) carbonate (12 mg, 0.0394 mmol, 1.28 equiv.), and DIPEA (32 μL, 0.184 mmol, 6.0 equiv.)) in DMF (1 mL) was stirred at RT for 1 h at which time 1-(2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)-1-(4-((S)-2-((S)-2-((tert-butoxycarbonyl)amino)-3-methylbutanamido)-5-ureidopentanamido)-2-((methylamino)methyl)benzyl)pyrrolidin-1-ium (50 mg, 0.0308 mmol, 1.0 equiv.) and additional DIEA (53.7 μL, 0.308 mmol, 10 equiv.) were added. After stirring for 1 hour the solution was diluted with DMSO (2.5 mL) and purified by RP-HPLC. After lyophilization, 1-(2-((((2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-5-((S)-2-((S)-2-((tert-butoxycarbonyl)amino)-3-methylbutanamido)-5-ureidopentanamido)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid was obtained. HRMS: (M+2H+)⁺²/2=1316.7200; Rt=2.64 min (5 min acidic method).

### Synthesis of 3-(1-(((1r,3s,5R,7S)-3-(2-((((4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid

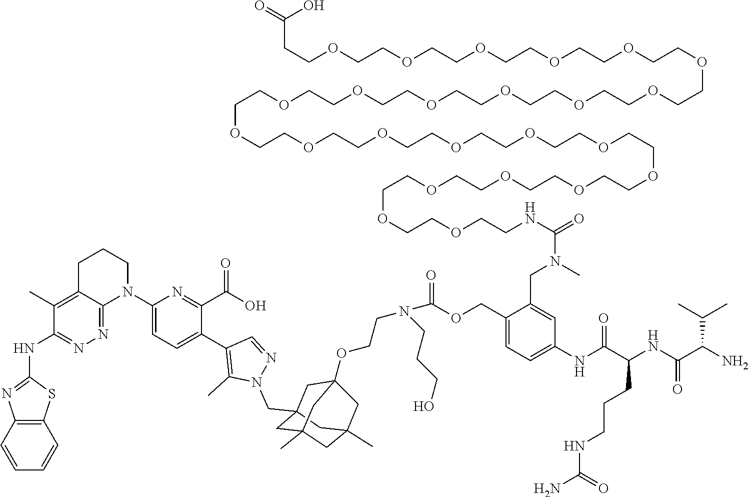 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](N)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

Following GENERAL PROCEDURE 4 with 1-(2-((((2-(((1s,3r,5R,7S)-3-((4-(6-(3-(benzo[d]thiazol-2-ylamino)-4-methyl-6,7-dihydropyrido[2,3-c]pyridazin-8(5H)-yl)-2-(((4-methoxybenzyl)oxy)carbonyl)pyridin-3-yl)-5-methyl-1H-pyrazol-1-yl)methyl)-5,7-dimethyladamantan-1-yl)oxy)ethyl)(3-hydroxypropyl)carbamoyl)oxy)methyl)-5-((S)-2-((S)-2-((tert-butoxycarbonyl)amino)-3-methylbutanamido)-5-ureidopentanamido)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid (39 mg, 0.014 mmol), 3-(1-(((1r,3s,5R,7S)-3-(2-((((4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid was obtained. General Procedure 4 was modified to clip small amount of TFA ester which formed on the primary hydroxyl. Upon concentration of TFA/CH2Cl2 the residue was dissolved in DMSO (1 mL), DIEA (125 μL, 50 equiv) was added followed by MeOH (1 mL). After standing 1 hour the ester was clipped and the solution was purified. HRMS: MH+=2412.3101; Rt=2.03 min (5 min acidic method).

### Synthesis of 6-(3-(benzo[d]thiazol-2-ylamino)-4-methyl-6,7-dihydropyrido[2,3-c]pyridazin-8(5H)-yl)-3-(1-(((1r,3s,5R,7S)-3-(2-((((2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(3-hydroxypropyl)amino)ethoxy)-5,7-dimethyladamantan-1-yl)methyl)-5-methyl-1H-pyrazol-4-yl)picolinic acid (L42C-P25)

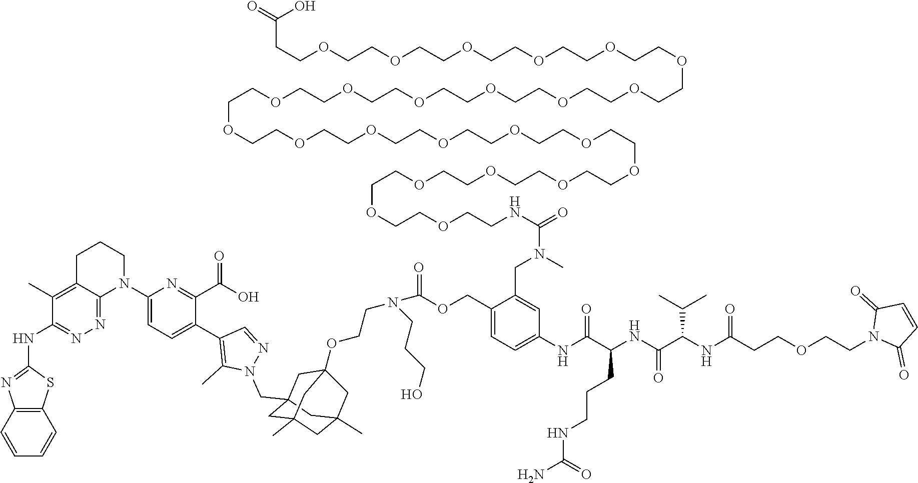 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1]

Following GENERAL PROCEDURE 5 with 3-(1-(((1r,3s,5R,7S)-3-(2-((((4-((S)-2-((S)-2-amino-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)oxy)carbonyl)(3-hydroxypropyl)amino)ethoxy)-5,7-dimethyladamantan-1-yl)methyl)-5-methyl-1H-pyrazol-4-yl)-6-(3-(benzo[d]thiazol-2-ylamino)-4-methyl-6,7-dihydropyrido[2,3-c]pyridazin-8(5H)-yl)picolinic acid (26 mg, 0.010 mmol) and 2,5-dioxopyrrolidin-1-yl 3-(2-(2,5-dioxo-2,5-dihydro-1H-pyrrol-1-yl)ethoxy)propanoate (4.0 mg, 0.013 mmol, 1.25 equiv.), 6-(3-(benzo[d]thiazol-2-ylamino)-4-methyl-6,7-dihydropyrido[2,3-c]pyridazin-8(5H)-yl)-3-(1-(((1r,3s,5R,7S)-3-(2-((((2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)oxy)carbonyl)(3-hydroxypropyl)amino)ethoxy)-5,7-dimethyladamantan-1-yl)methyl)-5-methyl-1H-pyrazol-4-yl)picolinic acid was obtained. HRMS: MH+=2607.3601; Rt=2.27 min (5 min acidic method).

The following compounds were prepared using procedures similar to those described for L38A-P21:

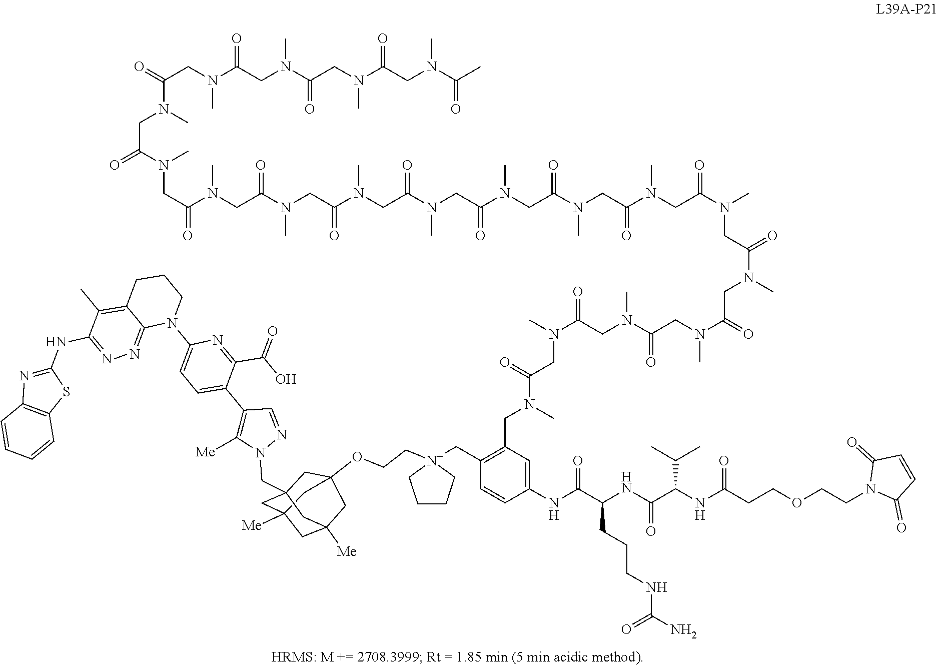 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1]

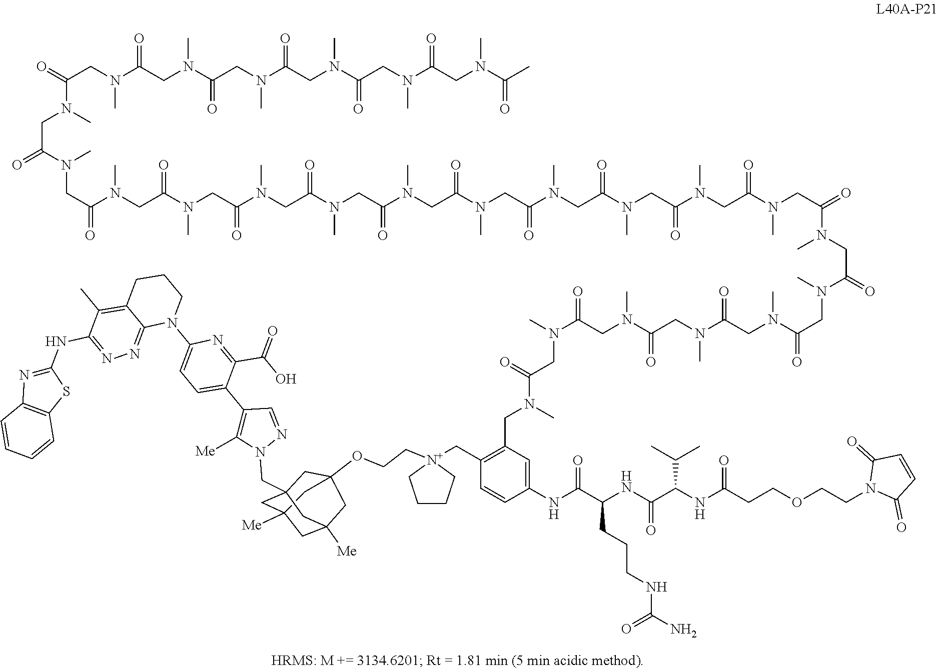 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1]

The following compounds could be prepared using procedures similar to those described above:
L11A-P1, L11A-P21, L11A-P27, L11C-P19, L11C-P25, L30A-P1, L30C-P19, L30A-P21, L30C-P25, L30A-P27, L35A-P1, L35C-P19, L35A-P21, L35C-P25, L35A-P27, L36A-P1, L36C-P19, L36A-P21, L36C-P25, L36A-P27, L37A-P1, L37C-P19, L37A-P21, L37C-P25, L37A-P27, L38A-P1, L38C-P19, L38A-P21, L38C-P25, L38A-P27, L39A-P1, L39C-P19, L39A-P21, L39C-P25, L39A-P27, L40A-P1, L40C-P19, L40A-P21, L40C-P25, L40A-P27, L42A-P1, L42C-P19, L42A-P21, L42C-P25, L42A-P27, L67A-P1, L67C-P19, L67A-P21, L67C-P25, L67A-P27, L100A-P1, L100C-P19, L100A-P21, L100C-P25, L100A-P27, L103A-P1, L103C-P19, L103A-P21, L103C-P25, L103A-P27, L111A-P1, L111C-P19, L111A-P21, L111C-P25, and L111A-P2. The structures of the compounds are shown in Table B.

### Example 5. Synthesis and Characterization of Bcl-xL Inhibitor ADCs

Exemplary antibody-drug conjugates (ADCs) were synthesized using the exemplary methods described below.

Abbreviations:

| | |
|---|---|
| Ab | antibody |
| ADC | antibody-drug conjugate |
| BCN | (N-[(1R,8S,9S)-bicyclo[6.1.0]non-4-yn-9- |
| | ylmethyloxycarbonyl]-1,8-diamino-3,6- |
| | dioxaoctane) |
| BTG | bacterial transglutaminase |
| CV | column volume |
| DAR | drug-to-antibody ratio |
| DBCO | dibenzo cyclooctyne |
| DFA | difluoroacetic acid |
| DMA | dimethylacetamide |
| DMF | dimethylformamide |
| DMSO | dimethyl sulfoxide |
| DTT | dithiothreitol |
| FA | formic acid |
| HIC | hydrophobic interaction chromatography |
| LC-MS | liquid chromatography mass spectrometry |
| L/P | linker-payload |
| mAb | monoclonal antibody |
| PBS | phosphate buffer saline |
| PES | polyether sulfone |
| PG | propylene glycol |
| PLRP-s | polymeric reverse phase column |
| rmp | reduction modifiable protein |
| SEC | size exclusion chromatography |
| TFA | trifluoroacetic acid |
| Tris | tris(hydroxymethyl)aminomethane |

### Materials and Methods: Conjugation and Analytical Characterization of ADCs BCL-xL Antibodies Specifications

Exemplary antibody-drug conjugates (ADCs) were synthesized using the exemplary methods described below. The anti-EphA2 antibodies defined in Table 9 were used for the preparation of the exemplary ADCs. The anti-EphA2 1C1 antibody was first described in WO2007030642, which is incorporated by reference in its entirety. An IgG anti-chicken lysozyme antibody (i.e., 3207 DANAPA) was used for the preparation of an isotype control ADC.

TABLE 9
Antibodies used for the synthesis of the exemplified ADCs

| | SEQ ID NO. | SEQ ID NO. |
|---|---|---|
| Antibody | (Heavy Chain) | (Light Chain) |
| anti-EphA2 E152C S375C 1C1 | 37 | 41 |
| anti-EphA2 1C1 E152C S375C DANAPA | 39 | 41 |
| anti-EphA2 E152C S375C 1C1 (LC S10T) hIgG1 | 37 | 43 |
| (WT Heavy Chain) | | |
| anti-EphA2 E152C S375C 1C1 (LC S72T) hIgG1 | 37 | 45 |
| (WT Heavy Chain) | | |
| anti-EphA2 E152C S375C 1C1 (LC G77S) hIgG1 | 37 | 47 |
| (WT Heavy Chain) | | |
| anti-EphA2 E152C S375C 1C1 (LC | 37 | 49 |
| S10T_S72T_G77S) hIgG1 (WT Heavy Chain) | | |
| anti-EphA2 E152C S375C 1C1 (LC S93Q) hIgG1 | 37 | 51 |
| (WT Heavy Chain) | | |
| anti-EphA2 E152C S375C 1C1 (LC S93V) hIgG1 | 37 | 53 |
| (WT Heavy Chain) | | |
| anti-EphA2 E152C S375C 1C1 (LC S93A) hIgG1 | 37 | 55 |
| (WT Heavy Chain) | | |
| anti-EphA2 E152C S375C 1C1 (LC N92A) hIgG1 | 37 | 57 |
| (WT Heavy Chain) | | |
| anti-EphA2 E152C S375C 1C1 (LC N92Q) hIgG1 | 37 | 59 |
| (WT Heavy Chain) | | |
| anti-EphA2 1C1 hIgG1 | 74 | 41 |
| anti-EphA2_1C1_DAR4_LC_N92Q_S72T | 37 | 75 |
| anti-EphA2_1C1_DAR4_LC_N92Q_G77S | 37 | 76 |
| anti-EphA2_1C1_DAR4_LC_N92Q_S72T_G77S | 37 | 77 |

### In Vitro Cysmab ADC Preparation

Antibody (typically 5-10 mg) was incubated with rProtein A Sepharose resin (GE) at a ratio of 10 mg Ab to 1 ml resin in PBS for 15 minutes with mixing in an appropriately sized disposable column. Cysteine HCl was added to a final concentration of 20 mM and incubated with agitation for 30 min at room temperature to allow the reactive cysteines to be deblocked. The resin was rapidly washed with 50 column volumes PBS on a vacuum manifold in multiple additions. The resin was then resuspended in an equal volume PBS containing 250 nM CuCl₂. Reformation of antibody interchain disulfides was monitored by taking time points. At each time point, 25 μL of resin slurry was removed, 1 μL of 20 mM MC-valcit-MMAE was added, and the tube flicked several times. The resin was spun down, supernatant removed, and then eluted with 50 μL Antibody elution buffer (Thermo). The resin was pelleted and the supernatant analyzed by reverse phase chromatography using an Agilent PLRP-S 4000A 5 μm, 4.6×50 mm column (Buffer A is water, 0.1% TFA, Buffer B Acetonitrile, 0.1% TFA, column held at 80° C., Flowrate 1.5 ml/min; Gradient 0 minutes—30% B, 5 minutes—45% B, 6.5 min—100% B, 8 minutes—100% B, 10 minutes—30%).

Once determined that the antibody has reformed its interchain disulfide bonds, the resin was washed with 10 column volumes PBS and the resin was resuspended in an equal volume PBS and 12 equivalents of the appropriate linker-payload (20 mM) in DMSO was added and then incubated at room temperature for 2 hours. The resin was then washed with 50 column volumes PBS to remove excess linker-payload. The ADC was eluted from the protein A resin with antibody elution buffer. The ADC was then dialyzed into PBS. The material was then concentrated using a centrifugal concentrator using an Amicon Ultra-15, 50 KDa, regenerated cellulose (Millipore, UFC0905024), to 4.5 mg/ml and filtered sterilely through 0.22 μm sterile PVDF Filter, 25 mm (Millapore, SLGV013SL) and stored at 4° C. The following analyses were performed—analytical SEC to determine percent monomer, reduced mass spectroscopy to determine DAR, LAL test to determine endotoxin load and protein concentration was determined by A280 utilizing extinction coefficient and molecular weight of antibody. All in vitro materials were >90% monomer. Percent aggregation, as determined by comparison of the area of the high-molecular-weight peak absorbance at 210 and 280 nm with the area of the peak absorbance for monomeric ADC. HRMS data (protein method) indicated a dominant mass of the heavy chain+2 species, giving a DAR of ˜4.0 was calculated by comparing MS intensities of peaks for DAR1 DAR2 and DAR3 species.

General Methodology: Drug-to-antibody ratio (DAR) of exemplary ADCs was determined by liquid chromatography-mass spectrometry (LC/MS) according to the following method. For all LC methods, mobile phase A was purified MS grade water (Honeywell, LC015-1), mobile phase B was MS grade 80% Isopropanol (Honeywell LC323-1):20% acetonitrile (Honeywell, LC015-1), LC323-1), supplemented with 1% of formic acid (FA) (Thermo Scientific, 85178). The column temperature was set at 80° C. A general MS method was optimized for all ADCs synthesized. The column used for analysis was an Agilent PLRP-S 4000 A; 2.1×150 mm, 8 μm (Agilent, PL1912-3803). Flowrate used was 0.3 ml/min. The gradient used was 0-0.75 minute 95% A, 0.76-1.9 minute 75% A, 1.91-11.0 minute 50% A, 11.01-11.50 10% A, 11.51-13.50 minute 95% A, 13.51-18 minute 95% A on an Acuity Bio H-Class Quaternary UPLC (Waters). MS system was Xevo G2-XS QToF ESI mass spectrometer (Waters) and data acquired from 1.5-11 minutes and masses were analyzed between 15000-80000 daltons. DAR was determined from the deconvoluted spectra or UV chromatogram by summing the integrated MS (total ion current) or UV (280 nm) peak area of unconjugated and conjugated given species (mAb or associated fragment), weighted by multiplying each area by the number of drug attached. The summed, weighted areas were divided by the sum of total area and the results produced a final average DAR value for the full ADC.

Size exclusion chromatography (SEC): SEC was performed to determine the quality of the ADCs and aggregation percentage (%) after purification. The analysis was performed on analytical column Superdex 200 Increase 5/150 GL (GE Healthcare, 28990945) in isocratic conditions 100% PBS pH 7.2 ((Hyclone SH30028.03)), flow 0.45 ml/min for 8 minutes. The % aggregate fraction of the ADC sample was quantified based on the peak area absorbance at 280 nm. Calculation was based on the ratio between the high molecular weight eluent at 280 nm divided by the sum of peak area absorbance at the same wavelength of the high molecular weight and monomeric eluents multiplied by 100%. Data was acquired on an Agilent Bio-Inert 1260 HPLC outfitted with a Wyatt miniDAWN light scattering and Treos refractive index detectors (Wyatt Technologies, Santa Barbara, CA).

### In Vivo Cysmab ADC Preparation

Antibody (25-200 mg) was incubated with rProtein A Sepharose resin (Cytiva) at a ratio of 10 mg Ab to 1 ml resin in PBS for 15 minutes with mixing in an appropriately sized disposable column. Cysteine HCl was added to a final concentration of 20 mM and incubated with agitation for 30 min at room temperature to allow the reactive cysteines to be deblocked. The resin was rapidly washed with 50 column volumes phosphate buffered saline pH 7.2 (PBS) on a vacuum manifold in multiple additions. The resin was then resuspended in an equal volume PBS containing 250 nM CuCl₂. Reformation of antibody interchain disulfides was monitored by taking time points. At each time point, 25 μL of resin slurry was removed, 1 μL of 20 mM MC-valcit-MMAE was added, and the tube flicked several times. The resin was spun down, supernatant removed, and then eluted with 50 μL Antibody elution buffer (Thermo). The resin was pelleted and the supernatant analyzed by reverse phase chromatography using an Agilent PLRP-S 4000A 5 um, 4.6×50 mm column (Buffer A is water, 0.1% TFA, Buffer B Acetonitrile, 0.1% TFA, column held at 80° C., Flowrate 1.5 ml/min; Gradient 0 minutes—30% B, 5 minutes—45% B, 6.5 min—100% B, 8 minutes—100% B, 10 minutes—30%).

Once it was determined that the antibody has reformed its interchain disulfide bonds, the resin was washed with 10 column volumes PBS and the resin was resuspended in an equal volume PBS and 12 equivalents of the appropriate linker-payload (20 mM) in DMSO was added and then incubated at room temperature for 2 hours. The resin was then washed with 50 column volumes PBS to remove excess linker-payload. The ADC was eluted from the protein A resin with antibody elution buffer. The ADC was then dialyzed into PBS and preparative SEC using a 16/60 or 26/600 S200 increase pg SEC column (GE) with PBS as the mobile phase if needed. The material was then concentrated using a centrifugal concentrator using an Amicon Ultra-15, 50 KDa, regenerated cellulose (Millipore, UFC0905024), to 4.5 mg/ml and filtered sterilely through 0.22 μm sterile PVDF Filter, 25 mm (Millapore, SLGV013SL) and stored at 4° C. The following analyses were performed-analytical SEC to determine percent monomer, mass spectroscopy to determine DAR, LAL test to determine endotoxin load and protein concentration was determined by A280 utilizing extinction coefficient and molecular weight of antibody. All in vivo materials were >95% monomer. Aggregation was typically <10%. Percent aggregation, as determined by comparison of the area of the high-molecular-weight peak absorbance at 210 and 280 nm with the area of the peak absorbance for monomeric ADC. HRMS data (protein method) indicated a dominant mass of the heavy chain+2 species, giving a DAR of ˜4.0 was calculated by comparing MS intensities of peaks for DAR1 DAR2 and DAR3 species.

General Methodology: Drug-to-antibody ratio (DAR) of exemplary ADCs was determined by liquid chromatography-mass spectrometry (LC/MS) according to the following method. For all LC methods, mobile phase A was purified MS grade water (Honeywell, LC015-1), mobile phase B was MS grade 80% Isopropanol (Honeywell LC323-1):20% acetonitrile (Honeywell, LC015-1), LC323-1), supplemented with 1% of formic acid (FA) (Thermo Scientific, 85178). The column temperature was set at 80° C. A general MS method was optimized for all ADCs synthesized. The column used for analysis was an Agilent PLRP-S 4000 A; 2.1×150 mm, 8 μm (Agilent, PL1912-3803). Flowrate used was 0.3 ml/min. The gradient used was 0-0.75 minute 95% A, 0.76-1.9 minute 75% A, 1.91-11.0 minute 50% A, 11.01-11.50 10% A, 11.51-13.50 minute 95% A, 13.51-18 minute 95% A on an Acuity Bio H-Class Quaternary UPLC (Waters). MS system was Xevo G2-XS QToF ESI mass spectrometer (Waters) and data acquired from 1.5-11 minutes and masses were analyzed between 15000-80000 daltons. DAR was determined from the deconvoluted spectra or UV chromatogram by summing the integrated MS (total ion current) or UV (280 nm) peak area of unconjugated and conjugated given species (mAb or associated fragment), weighted by multiplying each area by the number of drug attached. The summed, weighted areas were divided by the sum of total area and the results produced a final average DAR value for the full ADC.

Size exclusion chromatography (SEC): SEC was performed to determine the quality of the ADCs and aggregation percentage (%) after purification. The analysis was performed on analytical column Superdex 200 Increase 5/150 GL (GE Healthcare, 28990945) in isocratic conditions 100% PBS pH 7.2 ((Hyclone SH30028.03)), flow 0.45 ml/min for 8 minutes. The % aggregate fraction of the ADC sample was quantified based on the peak area absorbance at 280 nm. Calculation was based on the ratio between the high molecular weight eluent at 280 nm divided by the sum of peak area absorbance at the same wavelength of the high molecular weight and monomeric eluents multiplied by 100%. Data was acquired on an Agilent Bio-Inert 1260 HPLC outfitted with a Wyatt miniDAWN light scattering and Treos refractive index detectors (Wyatt Technologies, Santa Barbara, CA).

Example 6. Evaluation of In Vitro Activities of Payload P21 and EphA2-L11A-P21 ADC Alone or in Combination with Trametinib in a Panel of Cancer Cell Lines

EphA2-L11A-P21 antibody drug conjugate (ADC) alone or in combination with Trametinib were tested against cancer cell lines obtained from ATCC (American Type Culture Collection) or from other commercial cell line vendors. EphA2-L11A-P21 ADC was prepared according to any of the conjugation methods described herein. The cells were cultured in media that is optimal for their growth at 5% CO₂, 37° C. in a tissue culture incubator. Prior to seeding for the proliferation assay, the cells were split at least 2 days before the assay to ensure optimal growth density. On the day of seeding, cells were lifted off tissue culture flasks using 0.25% trypsin. Cell viability and cell density were determined using a cell counter (Vi-Cell XR Cell Viability Analyzer, Beckman Coulter). Cells with higher than 85% viability were seeded in white clear bottom 384-well plates (Greiner cat #781098) at a density of 1000 cells per well in 50 μL of standard growth media. Plates were incubated at 37° C. overnight in a tissue culture incubator.

The ADCs were prepared in standard phosphate buffered solution to desired concentrations. A series of 10 dilutions were made for each ADC. The prepared drug treatments were then added to the cells resulting in final concentrations of 100 nM to 0.005 nM. Combination partner Trametinib (MEK inhibitor) was added at a fixed concentration. Acoustic transfer devices (Echo525, Echo550, Beckman Coulter) were used to add the ADCs or combination partners to the cells. Each treatment was tested in triplicate assay plates. Plates were incubated at 37° C. overnight or for 5 days in a tissue culture incubator. The ability of the ADCs to inhibit cell proliferation and survival was assessed using the Promega CellTiter-Glo® proliferation assay. Plates were incubated at room temperature for 20 minutes to stabilize luminescent signals prior to reading using a multimode plate reader (Pherastar, BMG). Luminescent counts of untreated cells were taken the day after seeding (Day 0 readings), and after 5 days of treatment (Day 5 readings). The Day 5 readings of the untreated cells were compared to the Day 0 readings. Assays with at least one cell doubling during the incubation period were considered valid. To evaluate the effect of the drug treatments, luminescent counts from wells containing untreated cells (100% viability) were used to normalize treated samples. The concentrations of treatment required to inhibit 50% of cell growth or survival (GI50) were calculated using a four parameter logistic regression equation. Amax is the (fitted) response value of the ‘maximal biological effect’, reached by fitted curve within the unmasked measured concentration range. The test results are shown in Tables 10, 11, and 12 below.

TABLE 10
GI50 and GI Amax of EphA2-L11A-P21 ADC
alone in a panel of cancer cell lines

| Cell Line Name | Linker Payload | Combination Partner | GI50 (μM) | GI Amax |
|---|---|---|---|---|
| aspc1 | EphA2-L11A-P21 | Single Agent | >0.1 | 72.67 |
| capan2 | EphA2-L11A-P21 | Single Agent | 0.00013 | 20.25 |
| dv90 | EphA2-L11A-P21 | Single Agent | >0.1 | 129.86 |
| ebc1 | EphA2-L11A-P21 | Single Agent | 0.00003 | −8.42 |
| hcc1143 | EphA2-L11A-P21 | Single Agent | 0.0001 | 32.5 |
| jhom2b | EphA2-L11A-P21 | Single Agent | 0.00177 | 45.86 |
| ncin87 | EphA2-L11A-P21 | Single Agent | 0.00033 | 31.95 |
| ocum1 | EphA2-L11A-P21 | Single Agent | 0.00018 | 4.31 |
| panc0327 | EphA2-L11A-P21 | Single Agent | 0.00025 | 30.2 |
| skhep1 | EphA2-L11A-P21 | Single Agent | 0.00005 | −51.44 |
| skmel31 | EphA2-L11A-P21 | Single Agent | 0.00009 | −1.66 |
| sw403 | EphA2-L11A-P21 | Single Agent | >0.1 | 51.22 |

TABLE 11
GI50 and GI Amax of EphA2-L11A-P21 ADC in combination
with Trametinib in a panel of cancer cell lines

| Cell Line Name | Linker Payload | Combination Partner | GI50 (μM) | GI Amax |
|---|---|---|---|---|
| aspc1 | EphA2-L11A-P21 | 10 nM_Trametinib | 0.00001 | 22.51 |
| capan2 | EphA2-L11A-P21 | 10 nM_Trametinib | 0.00001 | −51.16 |
| dv90 | EphA2-L11A-P21 | 10 nM_Trametinib | 0.00035 | 6.59 |
| ebc1 | EphA2-L11A-P21 | 10 nM_Trametinib | 0.00001 | −40.57 |
| hcc1143 | EphA2-L11A-P21 | 10 nM_Trametinib | 0.00006 | −21.25 |
| jhom2b | EphA2-L11A-P21 | 10 nM_Trametinib | 0.01372 | 42.26 |
| ncin87 | EphA2-L11A-P21 | 10 nM_Trametinib | 0.0008 | 17.04 |
| ocum1 | EphA2-L11A-P21 | 10 nM_Trametinib | 0.00001 | −96.61 |
| panc0327 | EphA2-L11A-P21 | 10 nM_Trametinib | 0.00002 | −38.02 |
| skhep1 | EphA2-L11A-P21 | 10 nM_Trametinib | ND | ND |
| skmel31 | EphA2-L11A-P21 | 10 nM_Trametinib | 0.00001 | −14.51 |
| sw403 | EphA2-L11A-P21 | 10 nM_Trametinib | 0.00001 | −85.99 |

TABLE 12
GI50 and GI Amax of payload P21 in a panel of cancer cell lines

| Cell Line Name | Payload | GI50 (μM) | GI Amax |
|---|---|---|---|
| aspc1 | P21 | 0.04217 | 2.18 |
| capan2 | P21 | 0.00438 | −81.72 |
| dv90 | P21 | 0.26583 | 16.76 |
| ebc1 | P21 | 0.00026 | −96.78 |
| hcc1143 | P21 | 0.00099 | −43.38 |
| jhom2b | P21 | 0.06881 | −27.63 |
| ncin87 | P21 | 0.00077 | −29.31 |
| ocum1 | P21 | 0.00049 | −94.77 |
| panc0327 | P21 | 0.00235 | −79.2 |
| skhep1 | P21 | 0.00005 | −75.34 |
| skmel31 | P21 | 0.00005 | −56.5 |
| sw403 | P21 | 0.00532 | −64.33 |

### Example 7. In Vivo Efficacy Studies of EphA2-DANAPA-L11C-P25 ADC, 3207-DANAPA-L11C-P25 Isotype Control ADC, and EphA2-DANAPA CysMab Control Antibody in EBC-1 Cells

### Materials and Methods

EBC-1 cells were cultured at 37° C. (atmosphere of 5% CO₂) in DMEM medium (Gibco 11965-084) supplemented with 10% FBS (HI-FBS #134K19, Tet-free). Panc03.27 cells were cultured at 37° C. (atmosphere of 5% CO₂) in RPMI-1640 medium (Gibco 11875-085) supplemented with 15% FBS and 10 units/mL recombinant human insulin. Treatment with 0.25% Trypsin (Gibco 25200-056) was used for sub-culturing. To establish EBC-1 and Panc03.27 xenografts, cells were harvested and re-suspended in a 1:1 v/v mixture of phosphate buffered saline and Matrigel. A total of 5×10⁶ EBC-1 cells and 10×10⁶ Panc03.27 cells were injected subcutaneously in the flanks of female nude mice (Charles River, USA) in a volume of 200 μL. Tumor growth was monitored regularly post cell inoculation, and animals were randomized into treatment groups (n=8) with a mean tumor volume of about 200 mm³.

EphA2-DANAPA-L11C-P25 ADC and 3207-DANAPA-L11C-P25 isotype control ADC can be synthesized using any of the ADC preparation methods disclosed therein.

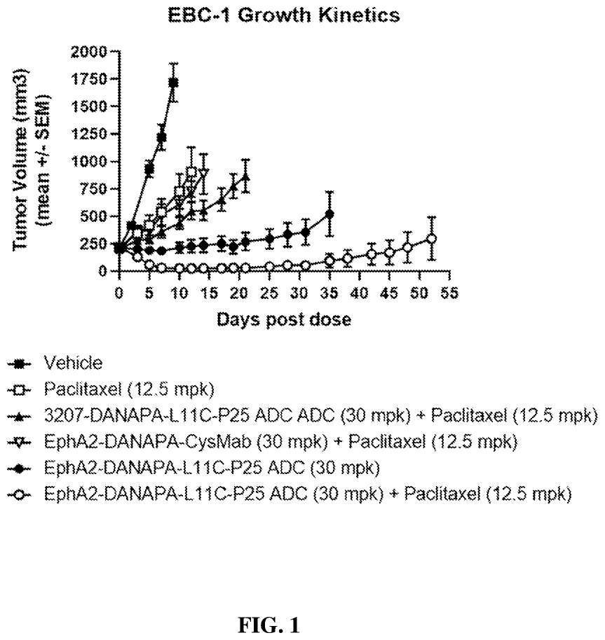

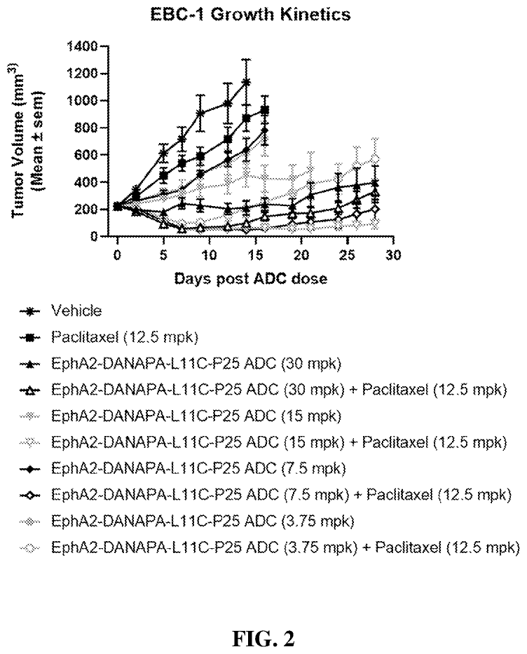

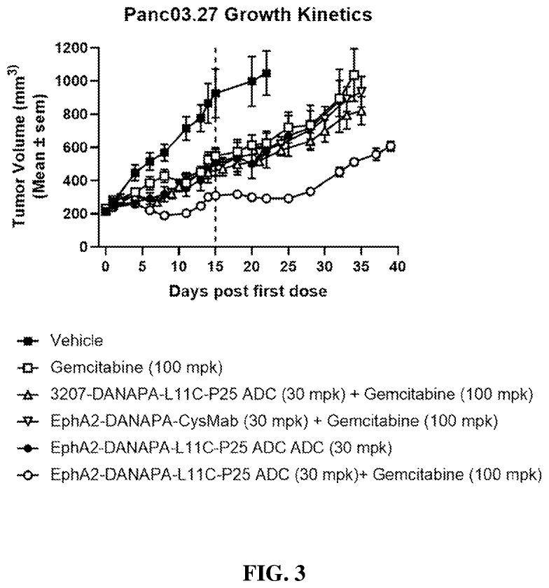

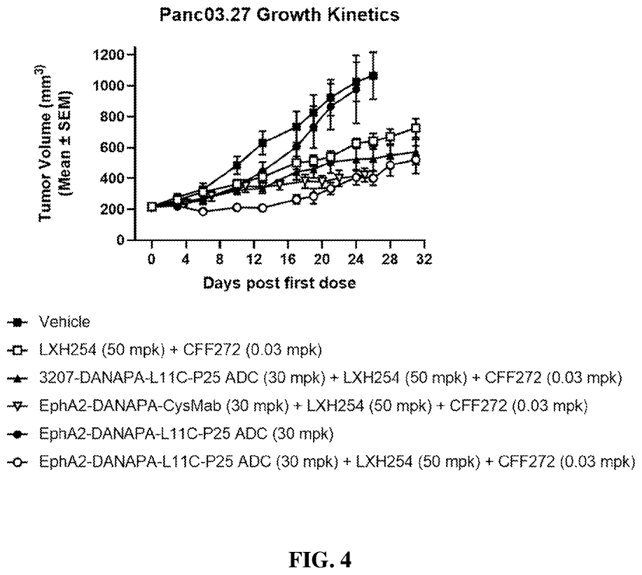

For EBC-1 efficacy studies, EphA2-DANAPA-L11C-P25 ADC, 3207-DANAPA-L11C-P25 isotype control ADC, and EphA2-DANAPA CysMab control antibody were all dosed in combination with paclitaxel (LC Laboratories, Woburn, MA, Cat #: P-9600), as indicated in FIG. 1 and FIG. 2. For Panc03.27 efficacy studies, EphA2-DANAPA-L11C-P25 ADC, 3207-DANAPA-L11C-P25 isotype control ADC, and EphA2-DANAPA CysMab control antibody were all dosed in combination with either gemcitabine (Hospira, NDC Code 0409-0185-01) or the MAPK inhibitors (MAPKi) LXH254 and CFF272, as indicated in FIG. 3 and FIG. 4 respectively. For studies testing the paclitaxel combination, the EphA2 and isotype control ADCs and EphA2 CysMab antibody were administered intravenously (IV) once at the start of treatment at the dose levels specified in FIG. 1 and FIG. 2, followed by a single IV dose of paclitaxel at 12.5 mg/kg 24 hours later. For studies testing the MAPK inhibitor combination, the EphA2 and isotype control ADCs and EphA2 CysMab antibody were administered intravenously (IV) once at the start of treatment at 30 mg/kg, followed by repeat dosing of LXH254 and CFF272 starting 24 hours later. LXH254 was administered orally (PO) at 50 mg/kg twice per day, while CFF272 was administered orally at 0.03 mg/kg once per day. For studies testing the gemcitabine combination, the EphA2 and isotype control ADCs and EphA2 CysMab antibody were administered intravenously (IV) on Days 0 and 14. The gemcitabine was administered intraperitoneally (IP) at 100 mg/kg on Days 1, 4, 7, 15, 18, and 21 (q3Dx3 after each ADC or CysMab dose). All reagents were dosed at 10 mL/kg based on the individual mouse body weight. The ADCs and CysMab were formulated accordingly in PBS on the day of treatment. Paclitaxel was formulated by first reconstituting in 50% Ethanol+50% Cremophor EL (Kolliphor EL) at a concentration of 6 mg/mL, and then further diluting down to 1.25 mg/mL with sterile saline (0.9% sodium chloride) prior to administration on the day of treatment. Gemcitabine was formulated at 10 mg/mL in saline prior to administration on the day of treatment.

Tumor volume data were analyzed for statistical significance relative to the EphA2-DANAPA-L11C-P25 ADC combination groups. Unpaired two-tailed T-tests were used to make comparisons between groups.

As a measure of efficacy, % T/C values were calculated according to the following formula: (Δtumor volume in experimental group/Δtumor volume in vehicle control group)*100. Tumor regression was calculated according to the following formula: (Δtumor volume in experimental group/tumor volume in experimental group at start)*100. Δtumor volumes represent mean tumor volumes on the measurement day minus the mean tumor volume at the start of treatment. Results are presented in the data table as mean±SEM.

### Results

EphA2-DANAPA-L11C-P25 ADC dosed at 30 mg/kg in combination with paclitaxel at 12.5 mg/kg significantly (p<0.05) reduced the growth of EBC-1 tumors compared to the vehicle control and paclitaxel alone groups. This ADC also had significantly greater anti-tumor activity than the 3207-DANAPA-L11C-P25 isotype control ADC, and the EphA2-DANAPA CysMab control antibody, when combined with paclitaxel. EphA2-DANAPA-L11C-P25 ADC was significantly more efficacious in combination with paclitaxel than as a monotherapy. The combination led to complete responses in 6/8 animals by day 31 post-ADC dose. EphA2-DANAPA-L11C-P25 ADC was also able to induce tumor regression as a monotherapy, albeit to a lesser extent as regressions were observed in only 2/8 animals by day 31 post-ADC dose. All results summarized here are presented in FIG. 1 and Table 13.

Tumor regressions were observed when EphA2-DANAPA-L11C-P25 ADC was dosed alone or in combination with paclitaxel at a dose of 30 mg/kg. Reducing the dose of ADC augmented the differential activity between the combination groups and their respective dose-matched ADC monotherapy counterparts. Reducing the dose to 15 mg/kg impaired monotherapy activity, as the ADC could no longer regress tumors or induce stasis, and instead only caused tumor growth delay. The activity decreased further when the ADC monotherapy dose was reduced to 7.5 mg/kg and 3.75 mg/kg. Despite losing monotherapy activity, combining the EphA2-DANAPA-L11C-P25 ADC with paclitaxel demonstrated that the anti-tumor activity could be maintained even at lower ADC doses. Tumors regressed in the 15 mg/kg, 7.5 mg/kg, and 3.75 mg/kg ADC plus paclitaxel groups to the same degree as the 30 mg/kg ADC plus paclitaxel group within one week after treatment. Tumor relapse occurred at the 3.75 mg/kg dose; however, the 7.5 mg/kg dose induced a rather durable response in combination with paclitaxel for a period of 28 days post-dose. This dataset suggests that the ADC dose can be reduced by at least four-fold in combination with paclitaxel without sacrificing activity, indicating the potential for an improved therapeutic window. All results summarized here are presented in FIG. 2 and Table 14.

EphA2-DANAPA-L11C-P25 ADC dosed at 30 mg/kg in combination with gemcitabine significantly (p<0.05) reduced the growth of Panc03.27 tumors compared to the vehicle control and gemcitabine alone groups. This ADC also had significantly greater anti-tumor activity than the 3207-DANAPA-L11C-P25 isotype control ADC, and the EphA2-DANAPA CysMab control antibody, when combined with gemcitabine. EphA2-DANAPA-L11C-P25 ADC was significantly more efficacious in combination with gemcitabine than as a monotherapy. Average tumor sizes slightly decrease in the EphA2-DANAPA-L11C-P25 ADC plus gemcitabine combination group, and no complete regressions were observed. Relapsing tumors in the EphA2-DANAPA-L11C-P25 ADC plus gemcitabine group were able to respond to a second round of dosing starting on Day 14 post treatment initiation. Tumor stasis was observed after the subsequent ADC and gemcitabine doses. All results summarized here are presented in FIG. 3 and Table 15.

EphA2-DANAPA-L11C-P25 ADC dosed at 30 mg/kg in combination with the MAPK inhibitors LXH254 (panRAF) and CFF272 (MEK) significantly (p<0.05) reduced the growth of Panc03.27 tumors compared to the vehicle control and MAPK alone groups. This ADC also had significantly greater anti-tumor activity than the 3207-DANAPA-L11C-P25 isotype control ADC, and the EphA2-DANAPA CysMab control antibody, when combined with MAPK inhibition, particularly within the first two weeks after dosing. EphA2-DANAPA-L11C-P25 ADC was significantly more efficacious in combination with than as a monotherapy. The maximum response observed was tumor stasis. All results summarized here are presented in FIG. 4 and Table 16.

TABLE 13
Summary of the antitumor activity of EphA2-DANAPA-L11C-P25 ADC in combination with paclitaxel. All ΔTumor
volume, % T/C, and % Regression values are presented as means, based off tumor measurements collected on the
days post-treatment specified below. T/C and regression values were calculated using formulas specified in the methods.
Complete responders were identified as animals with tumors that had regressed to 0 mm³ by the specified timepoints.
Statistical analyses were performed by comparing each treatment group to the EphA2-DANAPA-L11C-P25 ADC +
paclitaxel combination group. Day of evaluation is denoted below each value, in parentheses.

| | ΔTumor | | | | Significance vs EphA2- |
|---|---|---|---|---|---|
| Test agent | volume | | % | Complete | DANAPA-L11C-P25 |
| (Dose, schedule, and route) | (mm³ ± SEM) | % T/C | Regression | Responders | ADC + Paclitaxel |
| Vehicle | 1501 ± 174 | 100% | N/A | N/A | P = 1.38e−7 |
| | (Day 9) | (Day 9) | | | (Day 9) |
| Paclitaxel | 687 ± 224 | 46% | N/A | 0/8 | P = 0.0016 |
| (12.5 mg/kg, SD, IV) | (Day 12) | (Day 12) | | (Day 12) | (Day 12) |
| 3207-DANAPA-L11C-P25 ADC | 350 ± 203 | 23% | N/A | 0/7 | P = 7.11e−6 |
| (30 mg/kg, SD, IV) + | (Day 12) | (Day 12) | | (Day 21) | (Day 12) |
| Paclitaxel (12.5 mg/kg, SD, IV) | | | | | |
| EphA2-DANAPA-CysMab | 531 ± 96 | 35% | N/A | 0/8 | P = 4.21e−6 |
| (3.75 mg/kg, SD, iv) + | (Day 12) | (Day 12) | | (Day 14) | (Day 12) |
| Paclitaxel (12.5 mg/kg, SD, IV) | | | | | |
| EphA2-DANAPA-L11C-P25 ADC | 13 ± 59 | 1% | N/A | 2/8 | P = 0.0049 |
| (30 mg/kg, SD, IV) | (Day 12) | (Day 12) | | (Day 31) | (Day 12) |
| EphA2-DANAPA-L11C-P25 ADC | −193 ± 14 | N/A | −88% | 6/8 | N/A |
| (30 mg/kg, SD, iv) + | (Day 12) | | (Day 12) | (Day 31) | |
| Paclitaxel (12.5 mg/kg, SD, IV) | | | | | |

N/A, not applicable

TABLE 14
Summary of the antitumor activity of EphA2-DANAPA-L11C-P25 ADC in combination with paclitaxel. All ΔTumor
volume, % T/C, and % Regression values are presented as means, based off tumor measurements collected on the
days post-treatment specified below. T/C and regression values were calculated using formulas specified in the methods.
Complete responders were identified as animals with tumors that had regressed to 0 mm³ by the specified timepoints.
Statistical analyses were performed by comparing each treatment group to the Paclitaxel monotherapy group. Statistical
significance was also calculated between each combination group and its corresponding ADC dose-matched monotherapy
counterpart. Day of evaluation is denoted below each value, in parentheses.

| | | | | | | Significance |
|---|---|---|---|---|---|---|
| | | | | | | of combination |
| | ΔTumor | | | | | vs. Dose- |
| Test agent (Dose, | volume | | | Complete | Significance | Matched ADC |
| schedule, and route) | (mm³ ± SEM) | % T/C | % Regression | Responders | vs Paclitaxel | Monotherapy |
| Vehicle | 912 ± 168 | 100% | N/A | N/A | P = 0.1853 | N/A |
| | (Day 14) | (Day 14) | | | (Day 14) | |
| Paclitaxel | 649 ± 97 | 71% | N/A | 0/8 | N/A | N/A |
| (12.5 mg/kg, SD, IV) | (Day 14) | (Day 14) | | (Day 16) | | |
| EphA2-DANAPA-L11C-P25 ADC | −13 ± 51 | N/A | −6% | 0/8 | P = 3.14e−5 | N/A |
| (30 mg/kg, SD, IV) | (Day 14) | | (Day 14) | (Day 16) | (Day 14) | |
| EphA2-DANAPA-L11C-P25 ADC | 228 ± 87 | 25% | N/A | 0/8 | P = 0.0071 | N/A |
| (15 mg/kg, SD, IV) | (Day 14) | (Day 14) | | (Day 16) | (Day 14) | |
| EphA2-DANAPA-L11C-P25 ADC | 414 ± 84 | 45% | N/A | 0/8 | P = 0.0900 | N/A |
| (7.5 mg/kg, SD, IV) | (Day 14) | (Day 14) | | (Day 16) | (Day 14) | |
| EphA2-DANAPA-L11C-P25 ADC | 392 ± 115 | 43% | N/A | 0/8 | P = 0.1090 | N/A |
| (3.75 mg/kg, SD, IV) | (Day 14) | (Day 14) | | (Day 16) | (Day 14) | |
| EphA2-DANAPA-L11C-P25 ADC | −125 ± 23 | N/A | −56% | 1/8 | P = 1.97e−6 | P = 0.0669 |
| (30 mg/kg, SD, iv) + | (Day 14) | | (Day 14) | (Day 24) | (Day 14) | (Day 14) |
| Paclitaxel (12.5 mg/kg, SD, IV) | | | | | | |
| EphA2-DANAPA-L11C-P25 ADC | −157 ± 20 | N/A | −70% | 3/8 | P = 1.13e−6 | P = 0.0005 |
| (15 mg/kg, SD, iv) + | (Day 14) | | (Day 14) | (Day 24) | (Day 14) | (Day 14) |
| Paclitaxel (12.5 mg/kg, SD, IV) | | | | | | |
| EphA2-DANAPA-L11C-P25 ADC | −175 ± 17 | N/A | −78% | 4/8 | P = 8.14e−7 | P = 7.54e−6 |
| (7.5 mg/kg, SD, iv) + | (Day 14) | | (Day 14) | (Day 24) | (Day 14) | (Day 14) |
| Paclitaxel (12.5 mg/kg, SD, IV) | | | | | | |
| EphA2-DANAPA-L11C-P25 ADC | −18 ± 62 | N/A | −8% | 2/8 | P = 4.79e−5 | P = 0.0073 |
| (3.75 mg/kg, SD, iv) + | (Day 14) | | (Day 14) | (Day 24) | (Day 14) | (Day 14) |
| Paclitaxel (12.5 mg/kg, SD, IV) | | | | | | |

N/A, not applicable

TABLE 15
Summary of the antitumor activity of EphA2-DANAPA-L11C-P25 ADC in combination with gemcitabine. All ΔTumor
volume, % T/C, and % Regression values are presented as means, based off tumor measurements collected on the
days post-treatment specified below. T/C and regression values were calculated using formulas specified in the methods.
Complete responders were identified as animals with tumors that had regressed to 0 mm³ by the specified timepoints.
Statistical analyses were performed by comparing each treatment group to the EphA2-DANAPA-L11C-P25 ADC +
gemcitabine combination group. Day of evaluation is denoted below each value, in parentheses.

| | ΔTumor | | | | Significance vs EphA2- |
|---|---|---|---|---|---|
| Test agent | volume | | % | Complete | DANAPA-L11C-P25 |
| (Dose, schedule, and route) | (mm³ ± SEM) | % T/C | Regression | Responders | ADC + Gemcitabine |
| Vehicle | 500 ± 71 | 100% | N/A | N/A | P = 5.54e−6 |
| | (Day 11) | (Day 11) | | | (Day 11) |
| Gemcitabine | 153 ± 39 | 31% | N/A | 0/8 | P = 0.0006 |
| (100 mg/kg, IP, q3dx3) | (Day 11) | (Day 11) | | (Day 34) | (Day 11) |
| 3207-DANAPA-L11C-P25 ADC | 184 ± 44 | 37% | N/A | 0/7 | P = 0.0006 |
| (30 mg/kg, SD, IV) + | (Day 11) | (Day 11) | | (Day 35) | (Day 11) |
| Gemcitabine (100 mg/kg, IP, | | | | | |
| q3dx3) | | | | | |
| EphA2-DANAPA-CysMab | 194 ± 20 | 39% | N/A | 0/8 | P = 4.94e−7 |
| (30 mg/kg, SD, IV) + | (Day 11) | (Day 11) | | (Day 35) | (Day 11) |
| Gemcitabine (100 mg/kg, IP, | | | | | |
| q3dx3) | | | | | |
| EphA2-DANAPA-L11C-P25 ADC | 132 ± 50 | 26% | N/A | 2/8 | P = 0.0116 |
| (30 mg/kg, SD, IV) | (Day 11) | (Day 11) | | (Day 35) | (Day 11) |
| EphA2-DANAPA-L11C-P25 ADC | −19 ± 13 | N/A | −9% | 6/8 | N/A |
| (30 mg/kg, SD, IV) + | (Day 11) | | (Day 11) | (Day 39) | |
| Gemcitabine (100 mg/kg, IP, | | | | | |
| q3dx3) | | | | | |

N/A, not applicable

TABLE 16
Summary of the antitumor activity of EphA2-DANAPA-L11C-P25 ADC in combination with MAPK inhibitors
LXH254 and CFF272. All ΔTumor volume, % T/C, and % Regression values are presented as means,
based off tumor measurements collected on the days post-treatment specified below. T/C and regression
values were calculated using formulas specified in the methods. Complete responders were identified as
animals with tumors that had regressed to 0 mm³ by the specified timepoints. Statistical analyses
were performed by comparing each treatment group to the EphA2-DANAPA-L11C-P25 ADC +
LXH254 + CFF272 group. Day of evaluation is denoted below each value, in parentheses.

| | ΔTumor | | | | Significance vs EphA2- |
|---|---|---|---|---|---|
| Test agent | volume | | % | Complete | DANAPA-L11C-P25 |
| (Dose, schedule, and route) | (mm³ ± SEM) | % T/C | Regression | Responders | ADC + LXH254/CFF272 |
| Vehicle | 413 ± 76 | 100% | N/A | N/A | P = 9.65e−5 |
| | (Day 13) | (Day 13) | | | (Day 11) |
| LXH254 (50 mg/kg, PO, BID) + | 193 ± 26 | 47% | N/A | 0/8 | P = 1.98e−5 |
| CFF272 (0.03 mg/kg, PO, QD) | (Day 13) | (Day 13) | | (Day 31) | (Day 11) |
| 3207-DANAPA-L11C-P25 ADC | 119 ± 23 | 29% | N/A | 0/8 | P = 0.0007 |
| (30 mg/kg, SD, IV) + | (Day 13) | (Day 13) | | (Day 31) | (Day 11) |
| LXH254 (50 mg/kg, PO, BID) + | | | | | |
| CFF272 (0.03 mg/kg, PO, QD) | | | | | |
| EphA2-DANAPA-CysMab | 133 ± 43 | 32% | N/A | 0/8 | P = 0.0114 |
| (3.75 mg/kg, SD, IV) + | (Day 13) | (Day 13) | | (Day 25) | (Day 11) |
| LXH254 (50 mg/kg, PO, BID) + | | | | | |
| CFF272 (0.03 mg/kg, PO, QD) | | | | | |
| EphA2-DANAPA-L11C-P25 ADC | 228 ± 62 | 55% | N/A | 0/8 | P = 0.0025 |
| (30 mg/kg, SD, IV) | (Day 13) | (Day 13) | | (Day 24) | (Day 11) |
| EphA2-DANAPA-L11C-P25 ADC | −8 ± 19 | N/A | −4% | 0/8 | N/A |
| (30 mg/kg, SD, IV) + | (Day 13) | | (Day 13) | (Day 31) | |
| LXH254 (50 mg/kg, PO, BID) + | | | | | |
| CFF272 (0.03 mg/kg, PO, QD) | | | | | |

N/A, not applicable

General Methodology: Drug-to-antibody ratio (DAR) of exemplary ADCs was determined by liquid chromatography-mass spectrometry (LC/MS) according to the following method. For all LC methods, mobile phase A was purified MS grade water (Honeywell, LC015-1), mobile phase B was MS grade 80% Isopropanol (Honeywell LC323-1):20% acetonitrile (Honeywell, LC015-1), LC323-1), supplemented with 1% of formic acid (FA) (Thermo Scientific, 85178). The column temperature was set at 80° C. A general MS method was optimized for all ADCs synthesized. The column used for analysis was an Agilent PLRP-S 4000 A; 2.1×150 mm, 8 um (Agilent, PL1912-3803). Flowrate used was 0.3 ml/min. The gradient used was 0-0.75 minute 95% A, 0.76-1.9 minute 75% A, 1.91-11.0 minute 50% A, 11.01-11.50 10% A, 11.51-13.50 minute 95% A, 13.51-18 minute 95% A on an Acuity Bio H-Class Quaternary UPLC (Waters). MS system was Xevo G2-XS QTOF ESI mass spectrometer (Waters) and data acquired from 1.5-11 minutes and masses were analyzed between 15000-80000 daltons. DAR was determined from the deconvoluted spectra or UV chromatogram by summing the integrated MS (total ion current) or UV (280 nm) peak area of unconjugated and conjugated given species (mAb or associated fragment), weighted by multiplying each area by the number of drug attached. The summed, weighted areas were divided by the sum of total area and the results produced a final average DAR value for the full ADC.

Size exclusion chromatography (SEC) was performed to determine the quality of the ADCs and aggregation percentage (%) after purification. The analysis was performed on analytical column Superdex 200 Increase 5/150 GL (GE Healthcare, 28990945) in isocratic conditions 100% PBS pH 7.2 ((Hyclone SH30028.03)), flow 0.45 ml/min for 8 minutes. The % aggregate fraction of the ADC sample was quantified based on the peak area absorbance at 280 nm. Calculation was based on the ratio between the high molecular weight eluent at 280 nm divided by the sum of peak area absorbance at the same wavelength of the high molecular weight and monomeric eluents multiplied by 100%. Data was acquired on an Agilent Bio-Inert 1260 HPLC outfitted with a Wyatt miniDAWN light scattering and Treos refractive index detectors (Wyatt Technologies, Santa Barbara, CA).

Example 8. FACS Binding Studies with an Engineered HKB-11 Cell Line that Overexpresses EphA2

### Materials and Methods

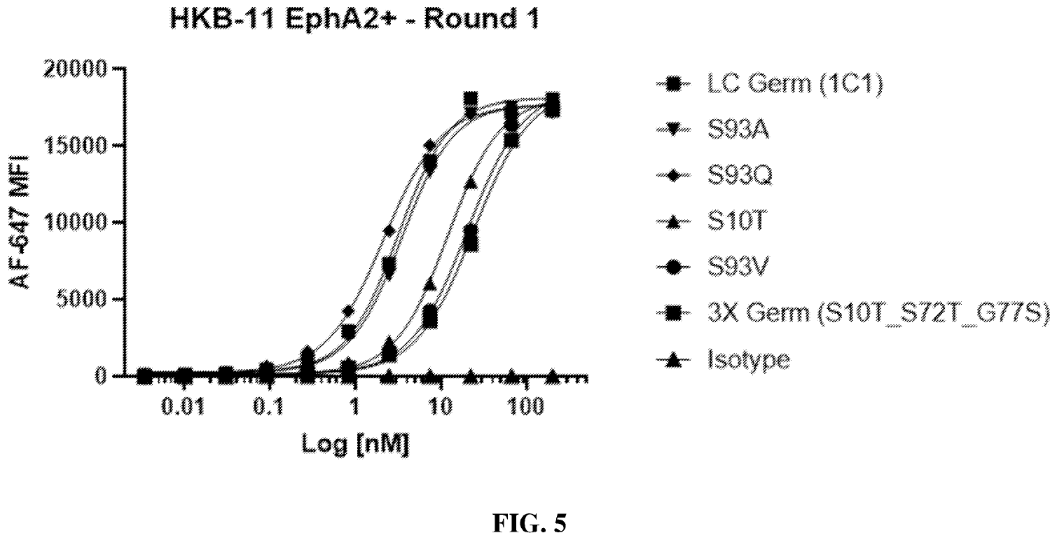

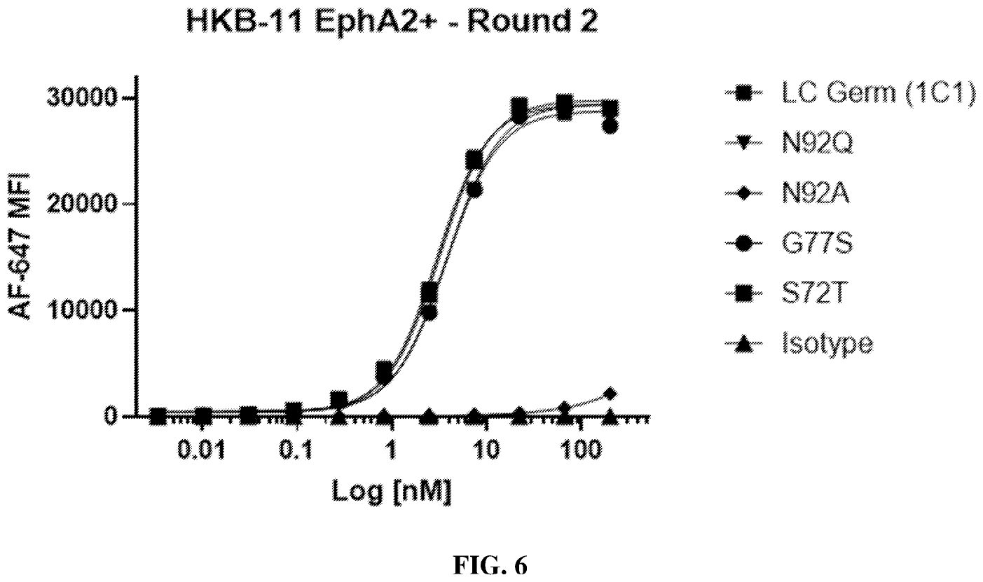

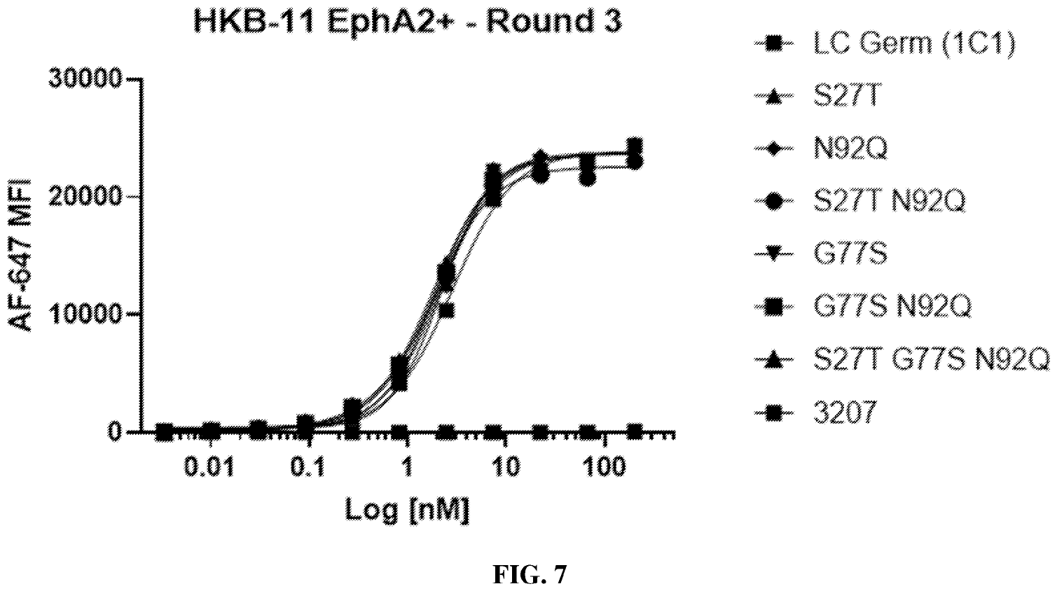

The binding affinity of EphA2 antibodies on cells was evaluated using flow cytometry (FACS). A dose titration of antibodies was incubated with an HKB-11 cell line that has been transduced to overexpress EphA2 (HKB-11: ATCC, CRL-12568) to determine EC₅₀ values for binding to cell surface expressed EphA2. Cell lines were cultured in media that is optimal for their growth at 5% CO₂, 37° C. in a tissue culture incubator. Cell viability and cell density were determined using a cell counter (Vi-Cell XR Cell Viability Analyzer, Beckman Coulter). Cells were plated at 200,000 cells/well in a 96-well U-bottom plate (Corning, #3799) and washed once with FACS buffer (MACSQuant® Running Buffer, #130-092-747). All subsequent steps were performed on ice and in ice cold FACS buffer to prevent internalization of the receptor. Cells were resuspended in 100 μL of FACS buffer containing an 11-point serial dilution of test antibodies at final concentrations of 200 nM-0.003 nM. Cells were incubated at 4° C. for 45 minutes. Following incubation, cells were washed three times in ice cold PBS at 1000 rpm for 5 minutes with 200 μL FACS buffer. Cells were then incubated with 100 μL of Alexa Fluor® 647 AffiniPure Goat Anti-Human IgG+IgM (H+L) (Jackson ImmunoResearch, #109-605-044) prepared in a 1:250 dilution in FACS buffer. Cells were incubated in the dark at 4° C. for 45 minutes. Following incubation, cells were washed three times in ice cold PBS at 1000 rpm for 5 minutes with 200 μL FACS buffer. Cells were then resuspended in 100 μL FACS buffer containing DAPI (ThermoFisher, #D1306) staining followed by readout via flow cytometry (MACSQuant® Analyzer 10). Data was analyzed using FlowJo v10.8.1 to obtain MFI (Mean Fluorescence Intensity) on live cells. EC₅₀ values were calculated using the GraphPad Prism 9 software. There were three rounds of experiments performed to evaluate three groupings of EphA2 antibodies. These rounds were performed on different days. All results summarized here are presented in FIGS. 5-7 and Table 17.

TABLE 17
Summary of Binding Results on HKB-11 EphA2+ Cell Line

| Antibody | Lot | EC₅₀ (nM) |
|---|---|---|
| LC Germ (1C1) | BA-71-QS06 | 3.15 |
| 3x germ | DC-69-ID09 | 23.87 |
| S93Q | DA-62-QP06 | 2.13 |
| S93V | PA-62-QD06 | 20.16 |
| S10T | BB-60-FO04 | 11.98 |
| S93A | HD-58-MP05 | 3.44 |
| LC Germ (1C1) | BA-71-QS06 | 2.76 |
| S72T | FE-76-NO13 | 3.19 |
| G77S | ZF-74-CQ11 | 3.73 |
| N92Q | DE-54-AX20 | 3.83 |
| N92A | BC-56-WI24 | n/a |
| LC Germ (1C1) | HE-38-AZ34 | 2.82 |
| S72T | DE-39-AD34 | 2.12 |
| G77S | ZE-39-AG34 | 2.21 |
| N92Q | VE-39-AK34 | 1.95 |
| S72T_N92Q | HA-13-AF39 | 1.8 |
| G77S_N92Q | HE-07-PV32 | 2.59 |
| S72T_G77S_N92Q | PE-07-PN32 | 1.81 |

### Example 9. KinExA Apparent Equilibrium K_{D} Determination

### Materials and Methods

Binding to human EphA2 expressed on HKB-11 cells for the anti-EphA2 IgG antibody 1C1 and its light chain point mutation IgGs was assessed in an avid equilibrium setting using KinExA. Free antibody in equilibrium titration reactions were measured by KinExA using a KinExA 3200, Autosampler (Sapidyne). Data were analyzed using KinExA Pro 4-5-X (Sapidyne).

Human EphA2 expressing HKB-11 cells were cultured in DMEM (Thermo Fisher cat #11995) with 10% heat inactivated FBS (HI-FBS, Thermo Fisher cat #10082) and 1% Pen/Strep (Thermo Fisher cat #15140). Cells were loosely adherent and were detached by gently tapping on the flask. Cells were pelleted by centrifugation (1,100 rpm, 4 min, RT) and resuspended in PBS with 10% HI-FBS to 2× the final density needed for equilibrium titration (typically 4e6 cells/ml). Cells were serially diluted one part to one part (1:1) in buffer 10 times to provide 10 concentration points.

Anti-EphA2 antibody was diluted in PBS with 10% HI-FBS and 0.04% sodium azide to 2× the final concentration of binding sites needed for equilibrium titration. For multicurve analysis, each data set required three antibody binding site concentrations: 1)<5-fold of K_{D} (K_{D}-controlled curve); 2)>15-fold of K_{D} (concentration-controlled curve); 3) a concentration in between (1) and (2), typically 5-fold higher than (1) and 10-fold lower than (2). Cells and antibody were combined 1:1 in an eppendorf tube and incubated overnight, 4° C., rotating. Buffer and antibody alone samples were included. Cells were pelleted and supernatants were transferred to a 2 ml 96-well deep well plate covered with pierceable film.

Polystyrene beads (Sapidyne cat #442178) were coated with 30 μg/ml anti-human IgG Fc capture antibody (Jackson cat #709-006-098) in PBS, rotating, overnight, at 4° C. Beads were pelleted by brief centrifugation, coating buffer was removed, and beads were blocked in PBS with 10 mg/ml BSA (EMD Millipore cat #126575) and 0.02% sodium azide for 1 hr, RT, rotating. Three tubes of prepared beads were added to 30 ml of running buffer (PBS, 0.02% sodium azide) in a glass bead vial.

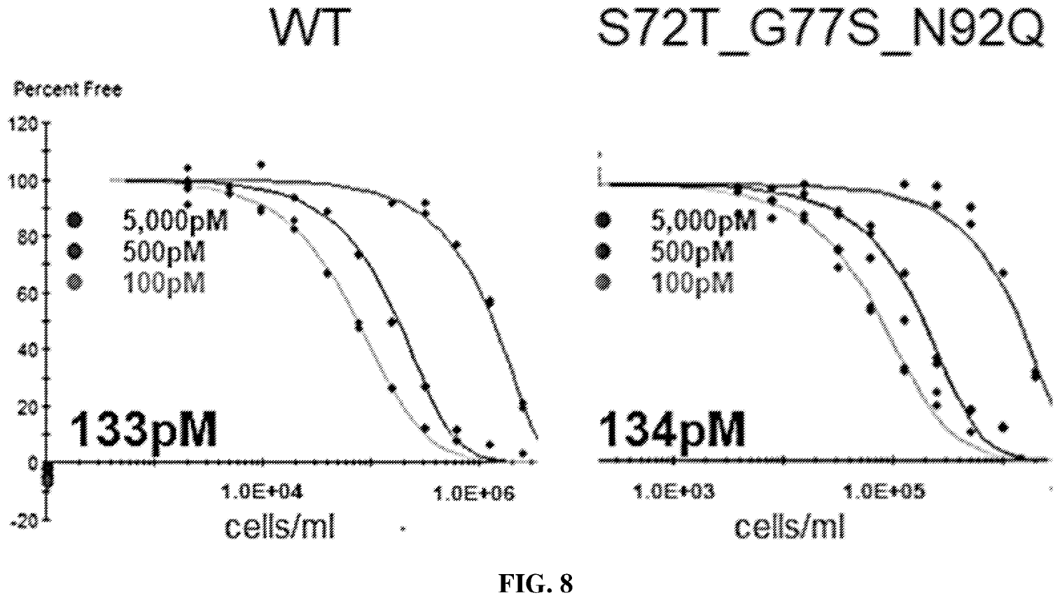

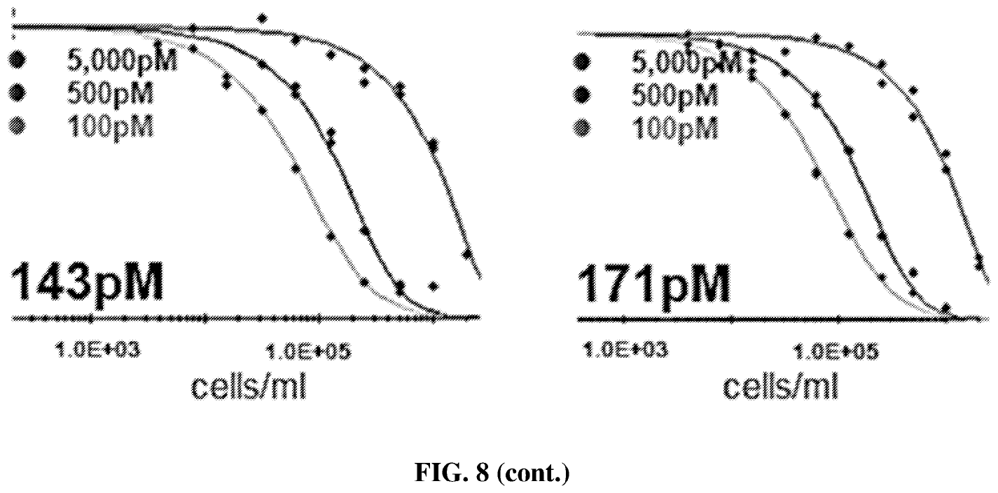

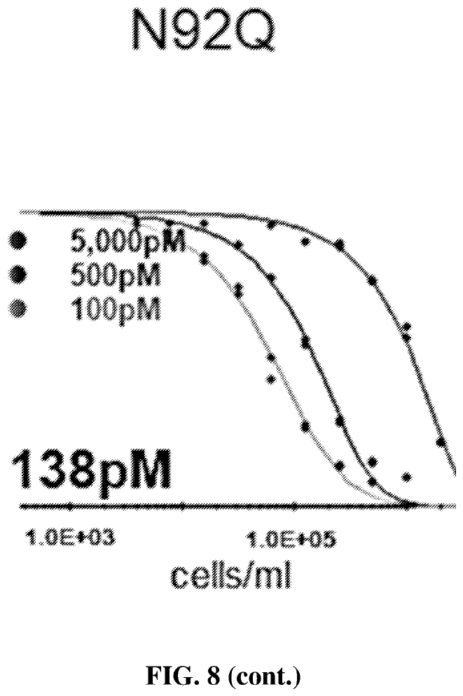

Equilibrium sample injection volume and label concentration were pre-determined to achieve 0.5V-2.0V with 500 μl injection of label (Gt-anti-Human H&L IgG Jackson cat #109-605-003). Data were analyzed by n-Curve Analysis. All results summarized here are presented in FIG. 8 and Table 18.

TABLE 18
Results of KinExA Apparent Equilibrium K_{D} Determination

| | Avid KD | | Average | Std. |
|---|---|---|---|---|
| Antibody | (pM) | error | pM | Dev. |
| 1C1 WT IgG | 158 | 2.6 | 179 | 59 |
| | 246 | 4.8 | | |
| | 133 | 3.4 | | |
| 1C1 S72T_G77S_N92Q IgG | 124 | 4.6 | 129 | 7 |
| | 134 | 6.7 | | |
| 1C1 S72T IgG | 132 | 3.5 | 137 | 8 |
| | 143 | 3.6 | | |
| 1C1 G77S IgG | 146 | 3.6 | 158 | 18 |
| | 171 | 3.3 | | |
| 1C1 N92Q IgG | 89 | 8.5 | 114 | 35 |
| | 138 | 3.3 | | |
| 1C1 Fab | >50,000 | | double | |
| | >20,000 | | digit nM | |

### Example 10. Biacore K_{D} Determination

### Materials and Methods

Binding to human, mouse and cyno EphA2 ectodomain to the anti-EphA2 IgG antibody 1C1 and its light chain point mutation IgGs was assessed using Biacore. Kinetic rate constants were determined via SPR using the Biacore 8k instrument (Cytiva, formerly GE Healthcare Lifesciences) as described below.

A human Fab capture method was utilized in order to determine kinetics for the antibodies. The anti-Fab antibody used was provided in the Human Fab capture kit (Cytiva, cat #28958325) and was immobilized on all 8 channels of CM5 sensor chip according to the manufacturer's instructions. Each channel has two flow cells. The reference flow cell contained only immobilized anti-Fab, whereas the reaction flow cell captured anti-EphA2 IgG through anti-Fab. HBS-EP+ Buffer, pH 7.6 was used as the running buffer. The soluble human, mouse and cyno EphA2 ectodomain flowed over both flow cells on 8 channels. The EphA2 ectodomain concentration started at 50 nM and was serially diluted at one part to one part (1:1) in the running buffer for six concentrations. The analysis chamber was kept at 37° C. Regeneration was performed at the end of each sample washing with the solution of 10 mM Glycine-HCl, pH 2.1 provided in the kit.

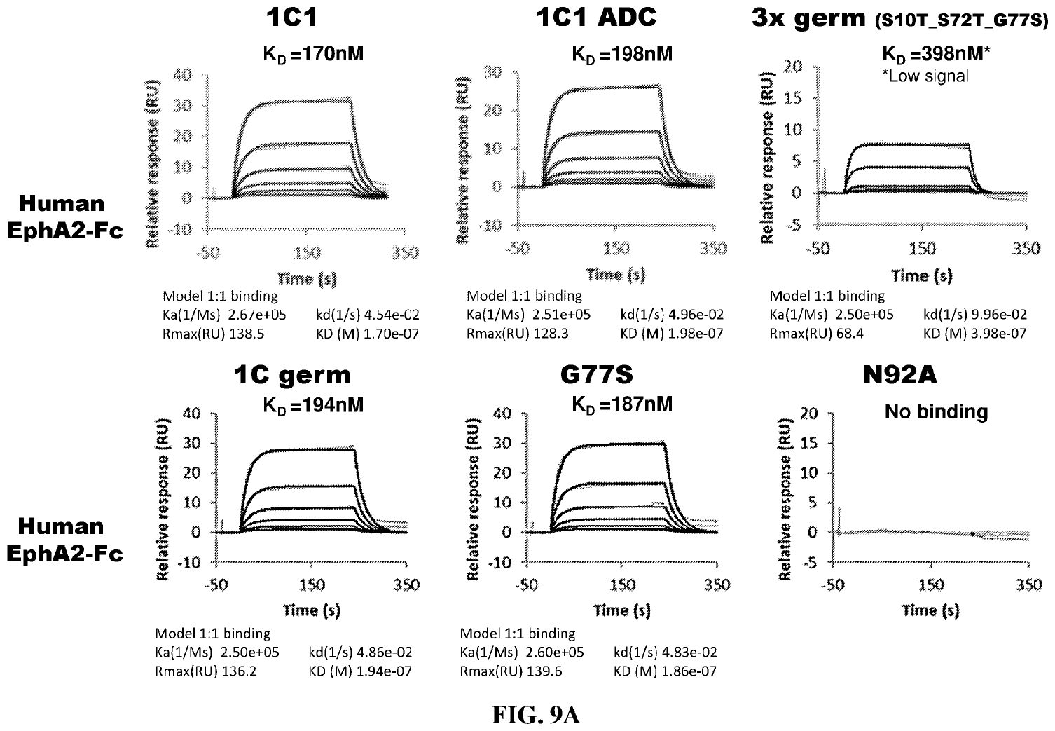

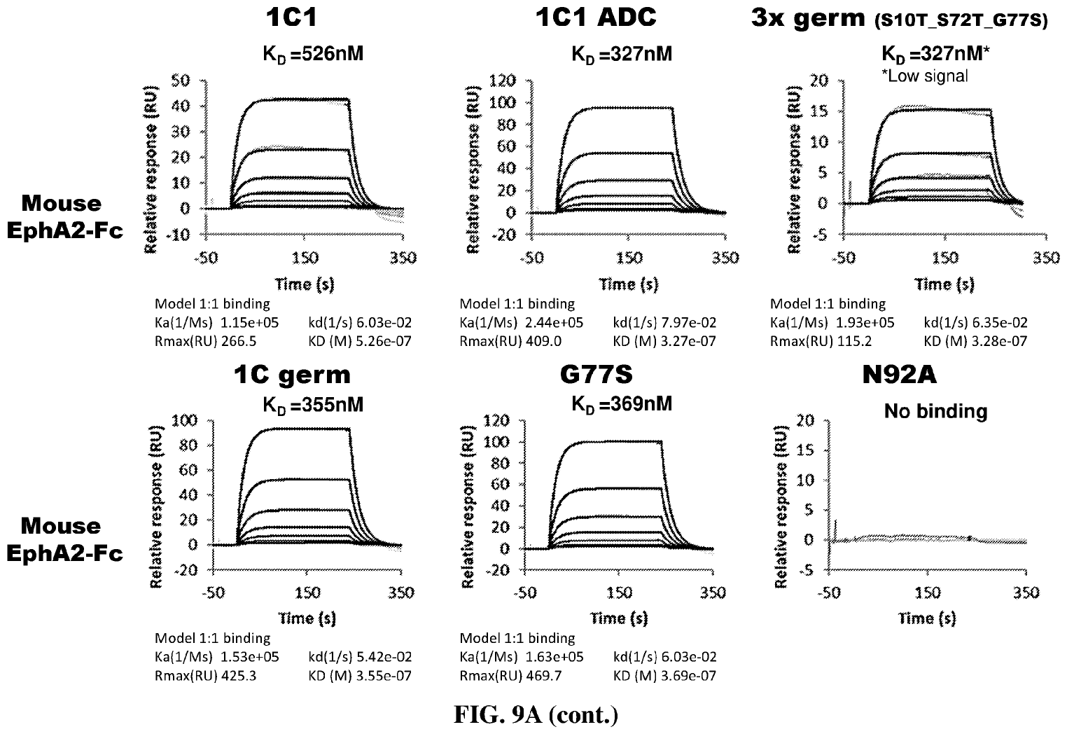

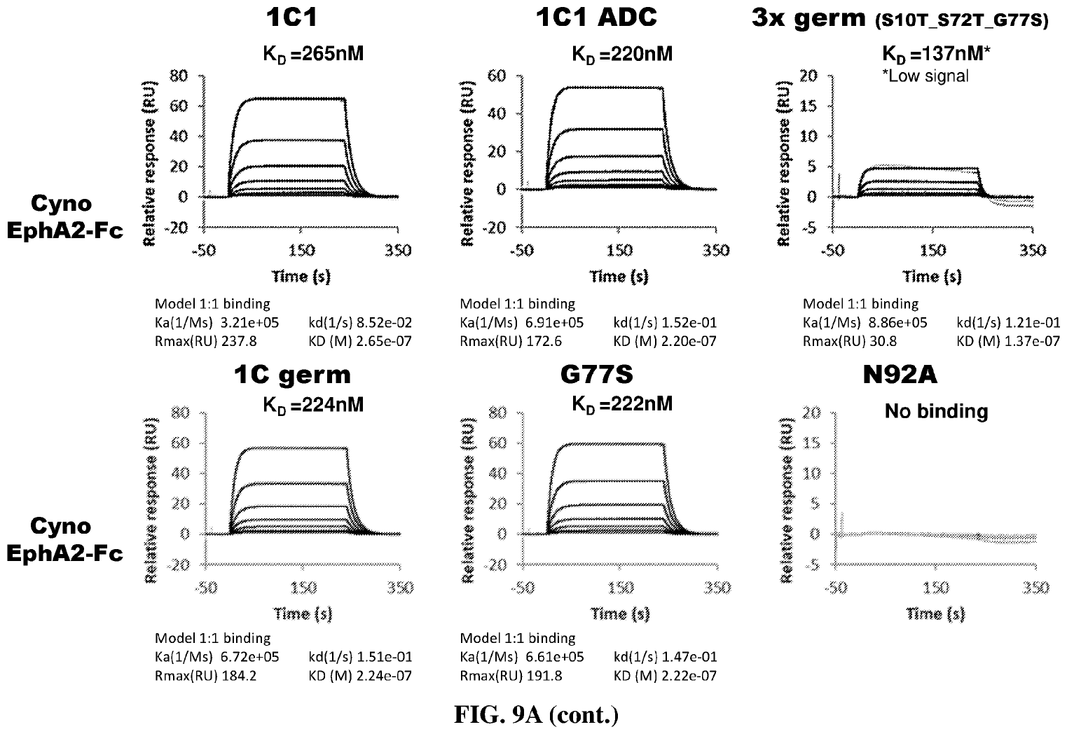

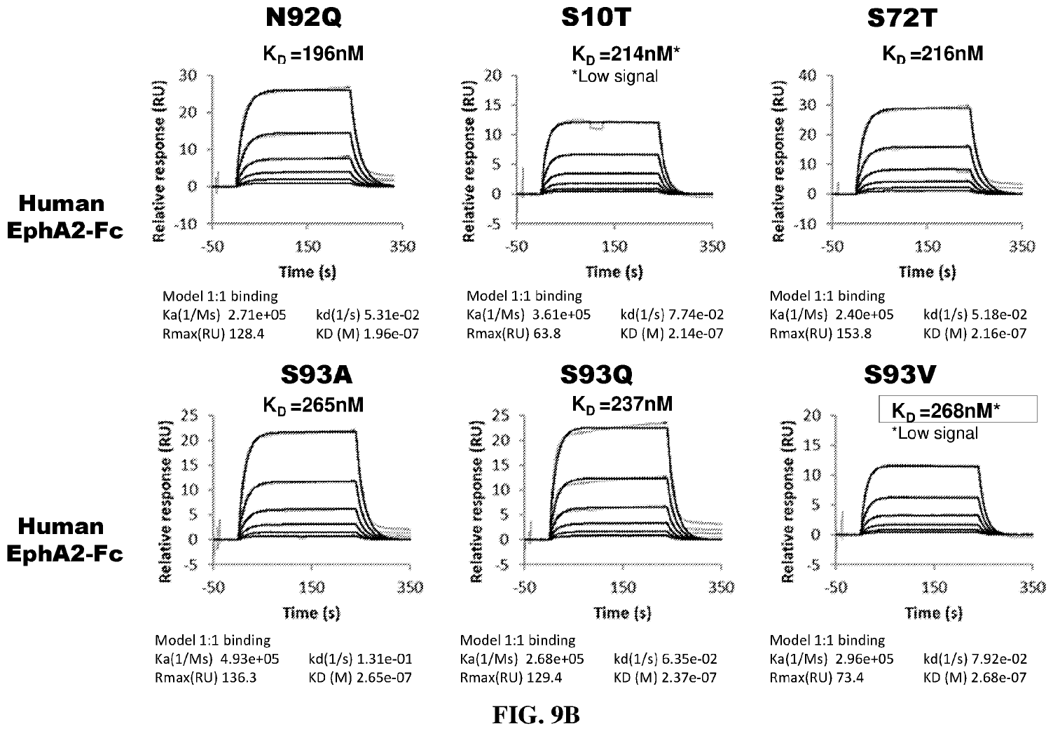

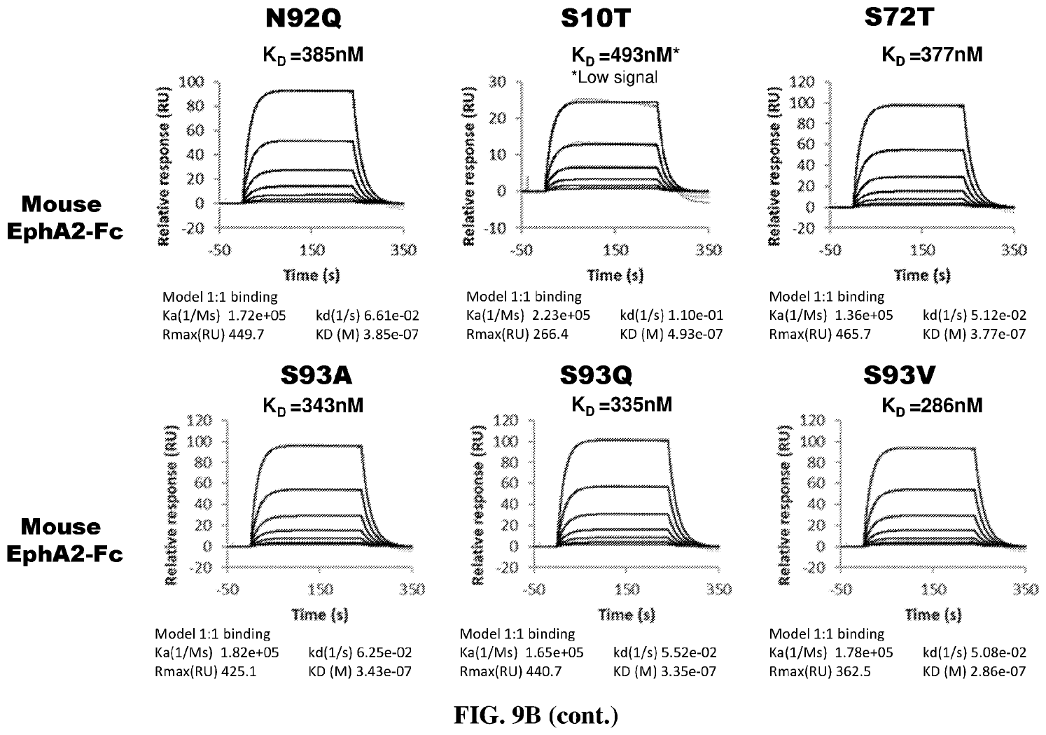

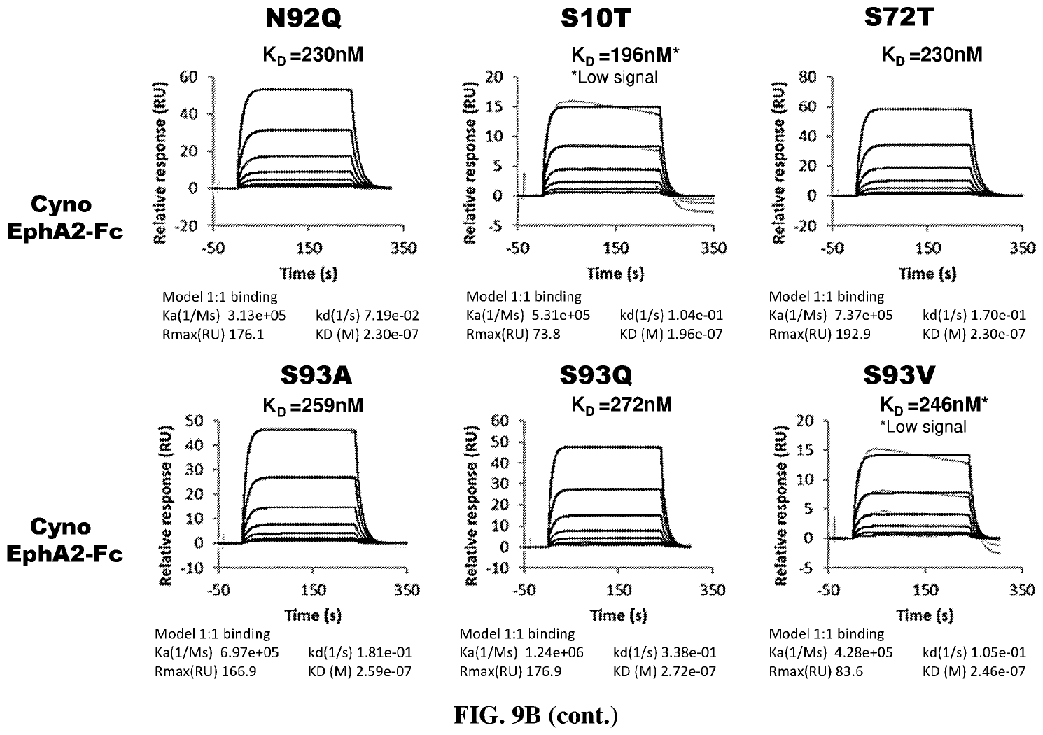

The data were analysed using the Biacore Insight Evaluation Software. Double reference subtraction was completed to generate the final data. The raw data was fitted to a 1:1 binding model, with parameter(s) Rₘₐₓ set to local. All results summarized here are presented in FIGS. 9A, 9B, and Table 19.

TABLE 19
Results of Biacore K_{d} Determination

| | | human EphA2 | | | mouse EphA2 | | | cyno EphA2 | | |
|---|---|---|---|---|---|---|---|---|---|---|
| Antibody | Sample ID | ka (1/Ms) | kd (1/s) | K_{D} (M) | ka (1/Ms) | kd (1/s) | K_{D} (M) | ka (1/Ms) | kd (1/s) | K_{D} (M) |
| 1C1 | iProt 120126 | 2.67E+05 | 4.54E−02 | 1.70E−07 | 1.15E+05 | 6.03E−02 | 5.26E−07 | 3.21E+05 | 8.52E−02 | 2.65E−07 |
| 1C1 ADC | XD-01-KE85 | 2.51E+05 | 4.96E−02 | 1.98E−07 | 1.66E+05 | 5.16E−02 | 3.11E−07 | 6.91E+05 | 1.52E−01 | 2.20E−07 |
| 3x germ | DC-69-ID09 | 2.50E+05 | 9.96E−02 | 3.98E−07* | 1.93E+05 | 6.35E−02 | 3.28E−07* | 8.86E+05 | 1.21E−01 | 1.37E−07* |
| (S10T | | | | | | | | | | |
| S72T G77S) | | | | | | | | | | |
| LC Germ | BA-71-QS06 | 2.50E+05 | 4.86E−02 | 1.94E−07 | 1.53E+05 | 5.42E−02 | 3.55E−07 | 6.72E+05 | 1.51E−01 | 2.24E−07 |
| G77S | ZF-74-CQ11 | 2.60E+05 | 4.83E−02 | 1.86E−07 | 1.63E+05 | 6.03E−02 | 3.69E−07 | 6.61E+05 | 1.47E−01 | 2.22E−07 |
| N92A | BC-56-WI24 | no binding | | | no binding | | | no binding | | |
| N92Q | DE-54-AX20 | 2.71E+05 | 5.31E−02 | 1.96E−07 | 1.72E+05 | 6.61E−02 | 3.85E−07 | 3.13E+05 | 7.19E−02 | 2.30E−07 |
| S10T | BB-60-FO04 | 3.61E+05 | 7.74E−02 | 2.14E−07* | 2.23E+05 | 1.10E−01 | 4.93E−07* | 5.31E+05 | 1.04E−01 | 1.96E−07* |
| S72T | FE-76-NO13 | 2.40E+05 | 5.18E−02 | 2.16E−07 | 1.36E+05 | 5.12E−02 | 3.77E−07 | 7.37E+05 | 1.70E−01 | 2.30E−07 |
| S93A | HD-58-MP05 | 4.93E+05 | 1.31E−01 | 2.65E−07 | 1.82E+05 | 6.25E−02 | 3.43E−07 | 6.97E+05 | 1.81E−01 | 2.59E−07 |
| S93Q | DA-62-QP06 | 2.68E+05 | 6.35E−02 | 2.37E−07 | 1.65E+05 | 5.52E−02 | 3.35E−07 | 1.24E+06 | 3.38E−01 | 2.72E−07 |
| S93V | PA-62-QD06 | 2.96E+05 | 7.92E−02 | 2.68E−07* | 1.78E+05 | 5.08E−02 | 2.86E−07 | 4.28E+05 | 1.05E−01 | 2.46E−07* |

*low binding signal

1. An antibody-drug conjugate of Formula (1): 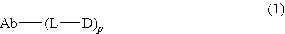
wherein Ab is an anti-EphA2 antibody or an antigen-binding fragment thereof;
L is a linker that covalently attaches Ab to D;
p is an integer from 1 to 16; and
D is a Bcl-xL inhibitor compound of Formula (I) or Formula (II) covalently attached to the linker L: 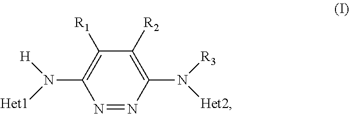
or an enantiomer, a diastereoisomer, and/or a pharmaceutically acceptable salt thereof, wherein:
R₁ and R₂ independently of one another represent a group selected from: hydrogen; linear or branched C₁-C₆alkyl optionally substituted by a hydroxyl or a C₁-C₆alkoxy group; C₃-C₆cycloalkyl; trifluoromethyl; linear or branched C₁-C₆alkylene-heterocycloalkyl wherein the heterocycloalkyl group is optionally substituted by a linear or branched C₁-C₆alkyl group;
or R₁ and R₂ form with the carbon atoms carrying them a C₃-C₆cycloalkylene group,
R₃ represents a group selected from: hydrogen; C₃-C₆cycloalkyl; linear or branched C₁-C₆alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and: 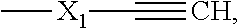
Rₐ and R_{b} independently of one another represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl groups; C₁-C₆alkylene-SO₂OH; C₁-C₆alkylene-SO₂O⁻; C₁-C₆alkylene-COOH; C₁-C₆alkylene-PO(OH)₂; C₁-C₆alkylene-NR_{d}Rₑ; C₁-C₆alkylene-N⁺R_{d}RₑR_{f}; C₁-C₆alkylene-phenyl wherein the phenyl may be substituted by a C₁-C₆alkoxy group;
the group: 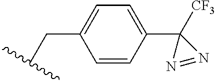 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₈heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈heterocycloalkyl,
Het₁ represents a group selected from: 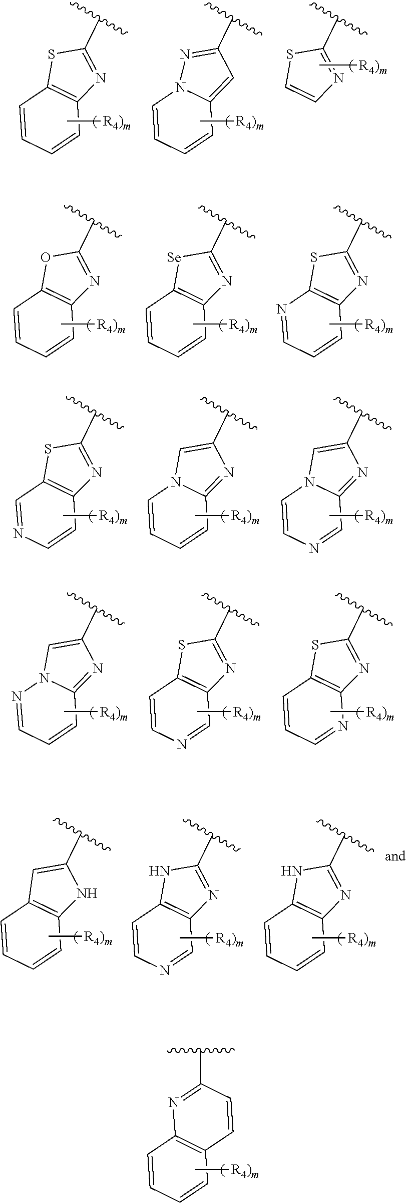
Het₂ represents a group selected from: 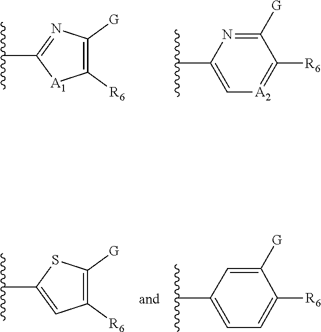
A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, C₁-C₆alkyl optionally substituted by a hydroxyl group, halogen, —NO₂, and —CN, in which:
R_{G1} and R_{G2} at each occurrence are each independently selected from the group consisting of hydrogen, C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl;
R_{G3} is selected from the group consisting of C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl; or
R_{G1} and R_{G2}, together with the atom to which each is attached are combined to form a C₃-C₈heterocycloalkyl; or in the alternative, G is selected from the group consisting of: 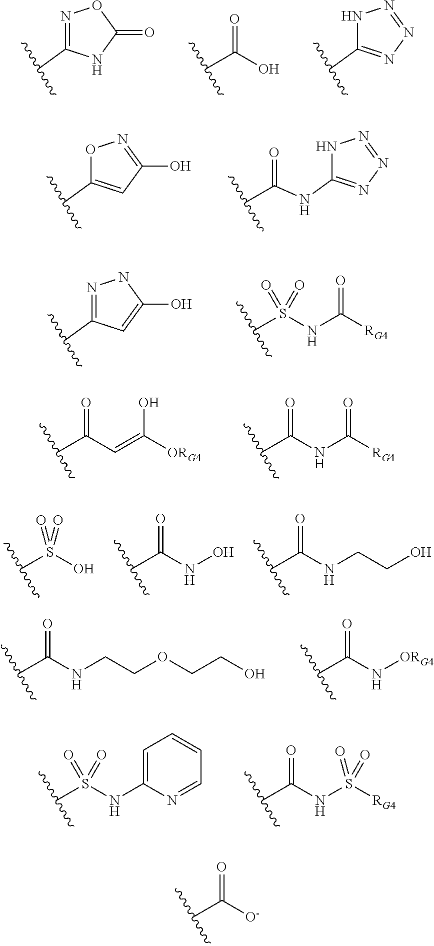
wherein R_{G4} is selected from hydrogen, C₁-C₆alkyl optionally substituted by 1 to 3 halogen atoms, C₂-C₆alkenyl, C₂-C₆alkynyl and C₃-C₆cycloalkyl,
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; C₂-C₆alkenyl; C₂-C₆alkynyl; halogen or —CN,
R₆ represents a group selected from:
hydrogen;
C₂-C₆alkenyl;
—X₂—O—R₇; 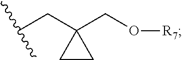
—X₂—NSO₂—R₇;
—C═C(R₉)—Y₁—O—R₇;
C₃-C₆cycloalkyl;
C₃-C₆heterocycloalkyl optionally substituted by a hydroxyl group;
C₃-C₆cycloalkylene-Y₂—R₇;
C₃-C₆heterocycloalkylene-Y₂—R₇ group,
an heteroarylene-R₇ group optionally substituted by a linear or branched C₁-C₆alkyl group,
R₇ represents a group selected from: linear or branched C₁-C₆alkyl group;
(C₃-C₆) cycloalkylene-R₈; or: 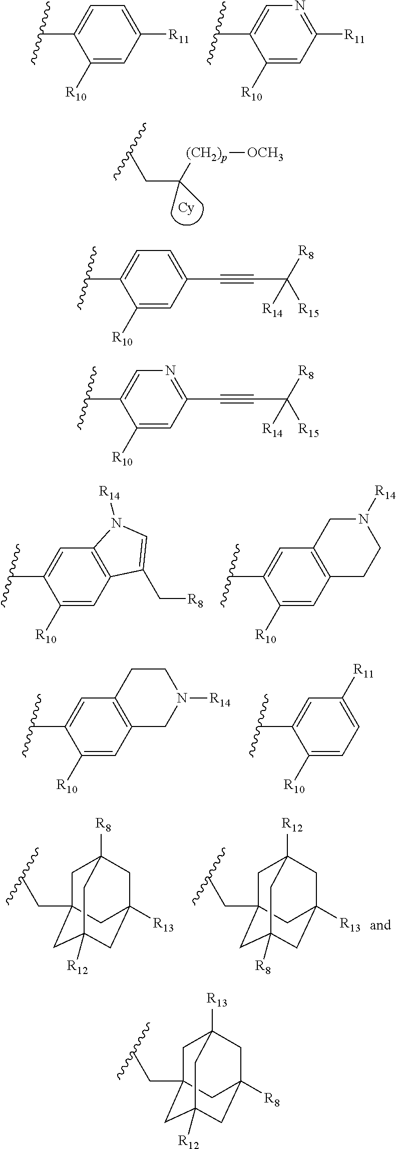
wherein Cy represents a C₃-C₈cycloalkyl,
R₈ represents a group selected from: hydrogen; linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and: 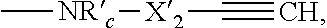
R₉ represents a group selected from linear or branched C₁-C₆alkyl, trifluoromethyl, hydroxyl, halogen, C₁-C₆alkoxy,
R₁₀ represents a group selected from hydrogen, fluorine, chlorine, bromine, —CF₃ and methyl,
R₁₁ represents a group selected from hydrogen, C₁-C₃alkylene-R₈, —O—C₁-C₃alkylene-R₈, —CO—NRₕRᵢ and —CH═CH—C₁-C₄alkylene-NRₕRᵢ, —CH═CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O⁻; C₁-C₆alkylene-COOH; C₁-C₆alkylene-PO(OH)₂; C₁-C₆alkylene-NR′_{d}R′ₑ; C₁-C₆alkylene-N⁺R′_{d}R′ₑR′_{f}; C₁-C₆alkylene-O—C₁-C₆alkylene-OH; C₁-C₆alkylene-phenyl wherein the phenyl may be substituted by a hydroxyl or a C₁-C₆alkoxy group;
the group: 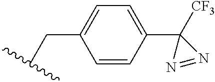 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, —NH—SO₂—,
m=0, 1 or 2,
p=1, 2, 3 or 4,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl,
wherein one of the R₃ and R₈ groups, if present, is covalently attached to the linker, and wherein the valency of an atom is not exceeded by virtue of one or more substituents bonded thereto; or 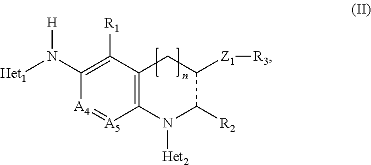
or an enantiomer, a diastereoisomer, and/or a pharmaceutically acceptable salt thereof, wherein:
n=0, 1 or 2, [symbol]
represents a single or a double bond,
A₄ and A₅ independently of one another represent a carbon or a nitrogen atom,
Z₁ represents a bond, —N(R)—, or —O—, wherein R represents a hydrogen or a linear or branched C₁-C₆alkyl,
R₁ represents a group selected from: hydrogen; linear or branched C₁-C₆alkyl optionally substituted by a hydroxyl or a C₁-C₆alkoxy group; C₃-C₆cycloalkyl; trifluoromethyl; linear or branched C₁-C₆alkylene-heterocycloalkyl wherein the heterocycloalkyl group is optionally substituted by a linear or branched C₁-C₆alkyl group;
R₂ represents a hydrogen or a methyl;
R₃ represents a group selected from: hydrogen; linear or branched C₁-C₄alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and: 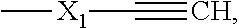
Rₐ and R_{b} independently of one another represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl groups; C₁-C₆alkylene-SO₂OH; C₁-C₆alkylene-SO₂O; C₁-C₆alkylene-COOH; C₁-C₆alkylene-PO(OH)₂; C₁-C₆alkylene-NR_{d}Rₑ; C₁-C₆alkylene-N⁺R_{d}RₑR_{f}; C₁-C₆alkylene-phenyl wherein the phenyl may be substituted by a C₁-C₆alkoxy group;
the group: 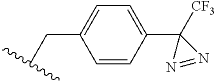 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₈heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈heterocycloalkyl,
Het₁ represents a group selected from: 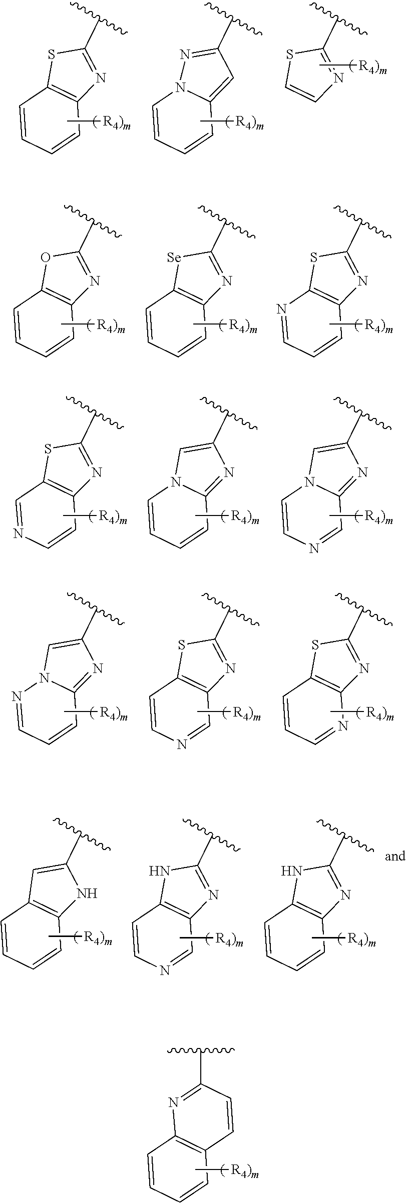
Het₂ represents a group selected from: 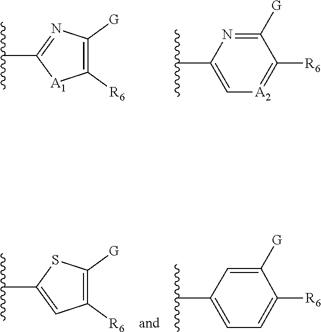
A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, C₁-C₆alkyl optionally substituted by a hydroxyl group, halogen, —NO₂, and —CN, in which:
R_{G1} and R_{G2} at each occurrence are each independently selected from the group consisting of hydrogen, C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl;
R_{G3} is selected from the group consisting of C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl; or
R_{G1} and R_{G2}, together with the atom to which each is attached are combined to form a C₃-C₈heterocycloalkyl; or in the alternative, G is selected from the group consisting of: 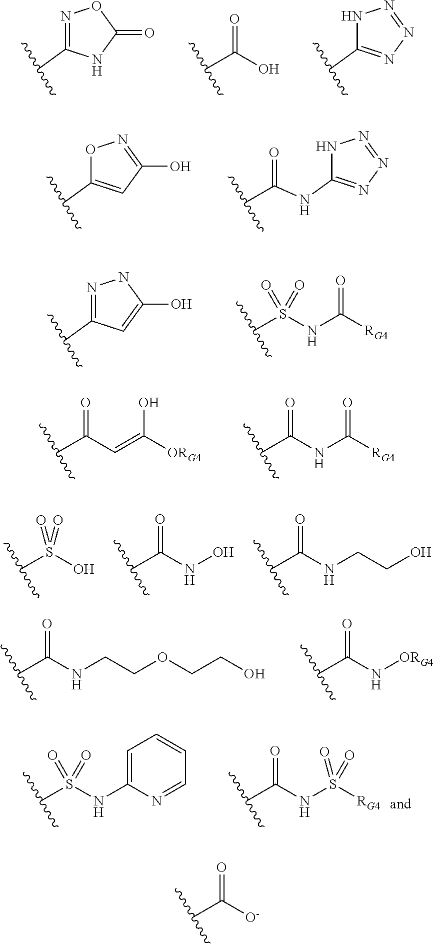
wherein R_{G4} is selected from hydrogen, C₁-C₆alkyl optionally substituted by 1 to 3 halogen atoms, C₂-C₆alkenyl, C₂-C₆alkynyl and C₃-C₆cycloalkyl,
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; C₂-C₆alkenyl; C₂-C₆alkynyl; halogen or —CN,
R₆ represents a group selected from:
hydrogen;
—C₂-C₆alkenyl;
—X₂—O—R₇; 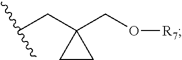
—X₂—NSO₂—R₇;
—C═C(R₉)—Y₁—O—R₇;
C₃-C₆cycloalkyl;
C₃-C₆heterocycloalkyl optionally substituted by a hydroxyl group;
C₃-C₆cycloalkylene-Y₂—R₇;
C₃-C₆heterocycloalkylene-Y₂—R₇ group,
an heteroarylene-R₇ group optionally substituted by a linear or branched C₁-C₆alkyl group,
R₇ represents a group selected from: linear or branched C₁-C₆alkyl group;
(C₃-C₆) cycloalkylene-R₈; or: 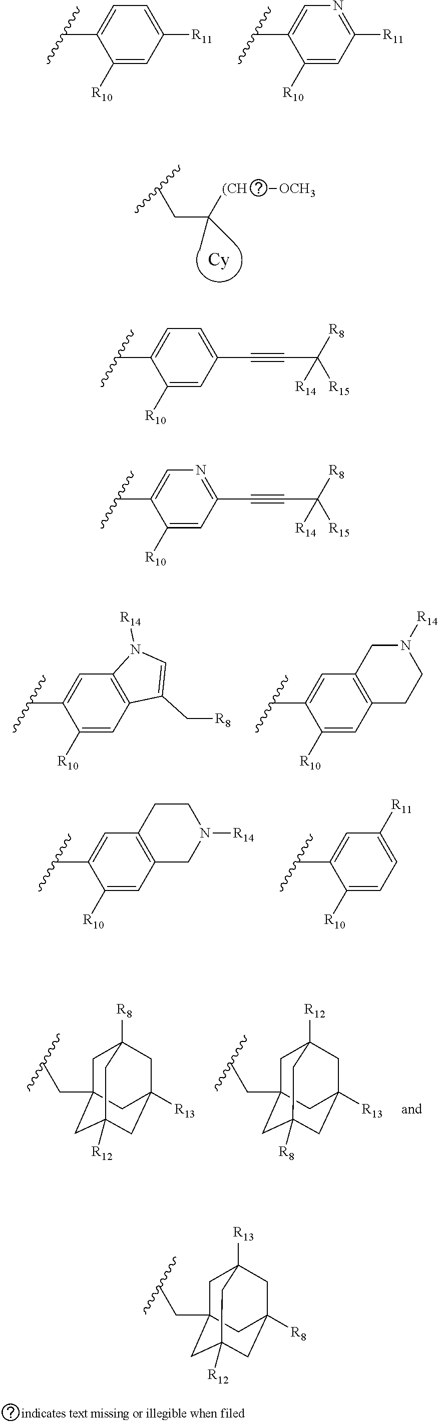
wherein Cy represents a C₃-C₈cycloalkyl,
R₈ represents a group selected from: hydrogen; linear or branched C₁-C₆alkyl, —NR′ₐR′_{b};
—NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and: 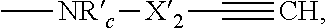
R₉ represents a group selected from linear or branched C₁-C₆alkyl, trifluoromethyl, hydroxyl, halogen, C₁-C₆alkoxy,
R₁₀ represents a group selected from hydrogen, fluorine, chlorine, bromine, —CF₃ and methyl,
R₁₁ represents a group selected from hydrogen, halogen, C₁-C₃alkylene-R₈, —O—C₁-C₃alkylene-R₈, —CO—NRₕRᵢ and —CH—CH—C₁-C₄alkylene-NRₕRᵢ, —CH═CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a
a cyclohexyl,
Rₕ and Rᵢ, independently of one another, represent a hydrogen or a linear or branched C₁-C₆alkyl group,
X₁ represents a linear or branched C₁-C₄alkylene group optionally substituted by one or two groups selected from trifluoromethyl, hydroxyl, halogen, C₁-C₆alkoxy,
X₂ represents a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O⁻; C₁-C₆alkylene-COOH; C₁-C₆alkylene-PO(OH)₂; C₁-C₆alkylene-NR′_{d}R′ₑ; C₁-C₆alkylene-N⁺R′_{d}R′ₑR′_{f}; C₁-C₆alkylene-O—C₁-C₆alkylene-OH; C₁-C₆alkylene-phenyl wherein the phenyl may be substituted by a hydroxyl or a C₁-C₆alkoxy group;
the group: 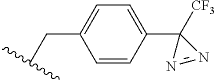 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, —NH—SO₂—,
m=0, 1 or 2,
p=1, 2, 3 or 4,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl,
wherein one of the R₃ and R₈ groups, if present, is covalently attached to the linker, and wherein the valency of an atom is not exceeded by virtue of one or more substituents bonded thereto; and
wherein the anti-EphA2 antibody or antigen-binding fragment thereof comprises:
(a) three heavy chain complementarity determining regions (HCDRs) comprising amino acid sequences of SEQ ID NO: 2 (HCDR1), SEQ ID NO: 3 (HCDR2), and SEQ ID NO: 4 (HCDR3); and three light chain complementarity determining regions (LCDRs) comprising amino acid sequences of SEQ ID NO: 12 (LCDR1), SEQ ID NO: 13 (LCDR2), and SEQ ID NO: 14, 26, 29, 32, or 35 (LCDR3);
(b) three heavy chain complementarity determining regions (HCDRs) comprising amino acid sequences of SEQ ID NO: 5 (HCDR1), SEQ ID NO: 6 (HCDR2), and SEQ ID NO: 4 (HCDR3); and three light chain complementarity determining regions (LCDRs) comprising amino acid sequences of SEQ ID NO: 15 (LCDR1), SEQ ID NO: 16 (LCDR2), and SEQ ID NO: 17, 24, 27, 30, 33, or 36 (LCDR3);
(c) three heavy chain complementarity determining regions (HCDRs) comprising amino acid sequences of SEQ ID NO: 7 (HCDR1), SEQ ID NO: 8 (HCDR2), and SEQ ID NO: 9 (HCDR3); and three light chain complementarity determining regions (LCDRs) comprising amino acid sequences of SEQ ID NO: 18 (LCDR1), SEQ ID NO: 13 (LCDR2), and SEQ ID NO: 17, 24, 27, 30, 33, or 36 (LCDR3); or
(d) three heavy chain complementarity determining regions (HCDRs) comprising amino acid sequences of SEQ ID NO: 10 (HCDR1), SEQ ID NO: 6 (HCDR2), and SEQ ID NO: 4 (HCDR3); and three light chain complementarity determining regions (LCDRs) comprising amino acid sequences of SEQ ID NO: 15 (LCDR1), SEQ ID NO: 16 (LCDR2), and SEQ ID NO: 17, 27, 30, 33, or 36 (LCDR3).

2. The antibody-drug conjugate of claim 1, wherein p is an integer from 1 to 6 or from 2 to 4, or p is 2 or 4; or p is determined by liquid chromatography-mass spectrometry (LC-MS).

3. The antibody-drug conjugate of claim 1, wherein L comprises:
an attachment group;
at least one bridging spacer group; and
at least one cleavable group, optionally at least one cleavable group comprising a pyrophosphate group and/or a self-immolative group;
optionally wherein the linker L comprises:
an attachment group,
at least one bridging spacer group,
a peptide group, and
at least one cleavable group.

4. The antibody-drug conjugate of claim 3, wherein -(L-D) is of the formula (A): 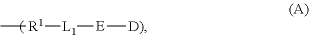
wherein:
R₁ is an attachment group;
L₁ is a bridging spacer group;
E is a cleavable group, optionally wherein: (a) the cleavable group comprises a pyrophosphate group or the cleavable group comprises 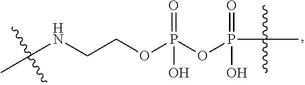 [SMILES: *NCCOP(=O)(O)OP(*)(=O)O]
and/or
(b) the bridging spacer group comprises:
(i) a polyoxyethylene (PEG) group;
(ii) a PEG group selected from, PEG1, PEG2, PEG3, PEG4, PEG5, PEG6, PEG7, PEG8, PEG9, PEG10, PEG11, PEG12, PEG13, PEG14, and PEG15;
(iii) a —CO—CH₂—CH₂-PEG12- group;
(iv) a butanoyl, pentanoyl, hexanoyl, heptanoyl, or octanoyl group; or
(v) a hexanoyl group.

5-6. (canceled)

7. The antibody-drug conjugate of claim 4, wherein (i) the attachment group is formed from at least one reactive group selected from a maleimide group, thiol group, cyclooctyne group, and an azido group; optionally wherein:
a) the maleimide group has the structure: 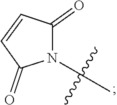 [SMILES: *N1C(=O)C=CC1=O]
b) the azido group has the structure: —N═N⁺═N⁻;
c) the cyclooctyne group has the structure: 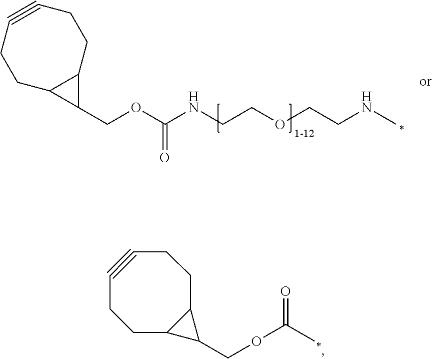
and wherein [symbol] is a bond to the antibody; or
d) the cyclooctyne group has the structure; 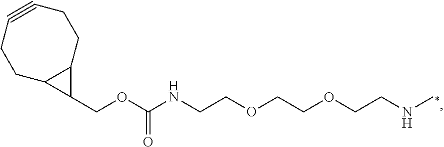 [SMILES: CNCCOCCOCCNC(=O)OCC1C2CCC#CCCC21]
and
wherein [symbol] is a bond to the antibody; or
(ii) the attachment group has a formula comprising: 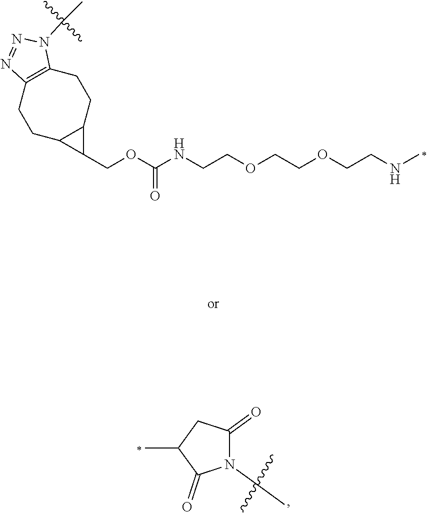
and
wherein [symbol] is a bond to the antibody; or
(iii) the antibody is joined to the linker (L) by an attachment group selected from: 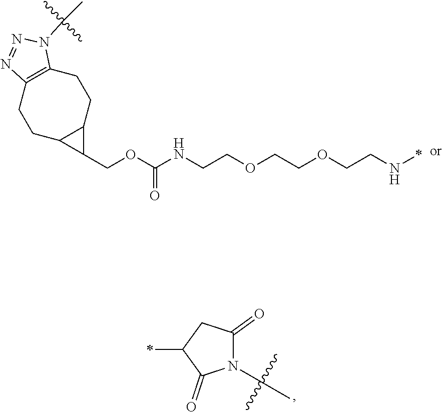
wherein [symbol] is a bond to the antibody, and wherein 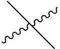
is a bond to the bridging spacer group, optionally, wherein (A) the bridging spacer group is —CO—CH₂—CH₂-PEG12 (B), wherein the bridging spacer group is joined to a cleavable group; optionally the cleavable group is -pyrophosphate-CH₂—CH₂—NH₂—; and/or (C) the cleavable group is joined to the Bel-xL inhibitor (D).

8-12. (canceled)

13. The antibody-drug conjugate of claim 3, wherein-(L-D) is of the formula (B): 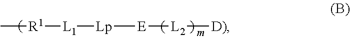
wherein:
R₁ is an attachment group;
L₁ is a bridging spacer;
Lp is a peptide group comprising 1 to 6 amino acid residues or Lp comprises a group 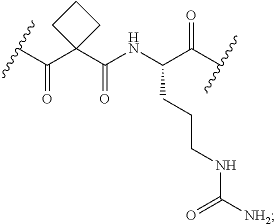 [SMILES: *C(=O)[C@H](CCCNC(N)=O)NC(=O)C1(C(*)=O)CCC1]
E is a cleavable group
L2 is a bridging spacer;
m is 0 or 1; and
D is a Bcl-xL inhibitor; optionally, wherein:
(i) the attachment group is formed from at least one reactive group comprising a maleimide group, thiol group, cyclooctyne group, and/or an azido group, optionally wherein:
a) the maleimide group has the structure: 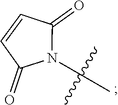 [SMILES: *N1C(=O)C=CC1=O]
b) the azido group has the structure: —N═N⁺═N⁻; or
c) the cyclooctyne group has the structure: 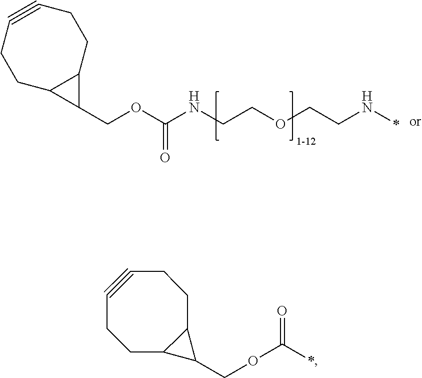
and wherein [symbol] is a bond to the antibody; or
(ii) the attachment group has a formula comprising: 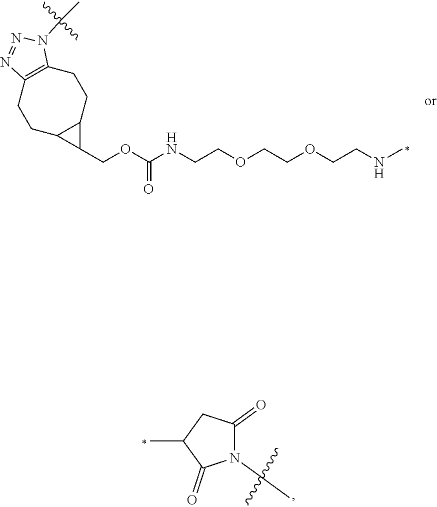
and
wherein [symbol] is a bond to the antibody.

14. (canceled)

15. The antibody-drug conjugate of claim 13, wherein:
(i) at least one bridging spacer comprises a PEG group, optionally the PEG group is selected from, PEG1, PEG2, PEG3, PEG4, PEG5, PEG6, PEG7, PEG8, PEG9, PEG10, PEG11, PEG12, PEG13, PEG14, and PEG15; or
(ii) at least one bridging spacer is selected from *—C(O)—CH₂—CH₂-PEG1-**, *—C(O)—CH₂-PEG3-**, *—C(O)—CH₂—CH₂-PEG12**, *—NH—CH₂—CH₂-PEG1-**, a polyhydroxyalkyl group, *—C(O)—N(CH₃)—CH₂—CH₂—N(CH₃)—C(O)—**, and *—C(O)—CH₂—CH₂-PEG12-NH—C(O)CH₂—CH₂—**, wherein ** indicates the point of direct or indirect attachment of the at least one bridging spacer to the attachment group and * indicates the point of direct or indirect attachment of the at least one bridging spacer to the peptide group.

16. The antibody-drug conjugate of claim 15, wherein L₁ is selected from *—C(O)—CH₂—CH₂-PEG1-**, *—C(O)—CH₂-PEG3-**, *—C(O)—CH₂—CH₂-PEG12**, *—NH—CH₂—CH₂-PEG1-**, and a polyhydroxyalkyl group, wherein ** indicates the point of direct or indirect attachment of L₁ to R¹ and * indicates the point of direct or indirect attachment of L₁ to Lp.

17. The antibody-drug conjugate of claim 16, wherein m is 1 and L2 is —C(O)—N(CH₃)—CH₂—CH₂—N(CH₃)—C(O)—.

18. The antibody-drug conjugate of claim 17, wherein
(i) the peptide group comprises 1 to 6, 1 to 4, 1 to 3 or 1 to 2 amino acid residues, optionally the amino acid residues are selected from glycine (Gly), L-valine (Val), L-citrulline (Cit), L-cysteic acid (sulfo-Ala), L-lysine (Lys), L-isoleucine (Ile), L-phenylalanine (Phe), L-methionine (Met), L-asparagine (Asn), L-proline (Pro), L-alanine (Ala), L-leucine (Leu), L-tryptophan (Trp), and L-tyrosine (Tyr);
(ii) the peptide group comprises Val-Cit, Val-Ala, Val-Lys, sulfo-Ala-Val-Cit, sulfo-Ala-Val-Ala, Gly-Gly-Gly, and/or Gly-Gly-Phe-Gly (SEQ ID NO: 78); or
(iii) the peptide group is selected from:

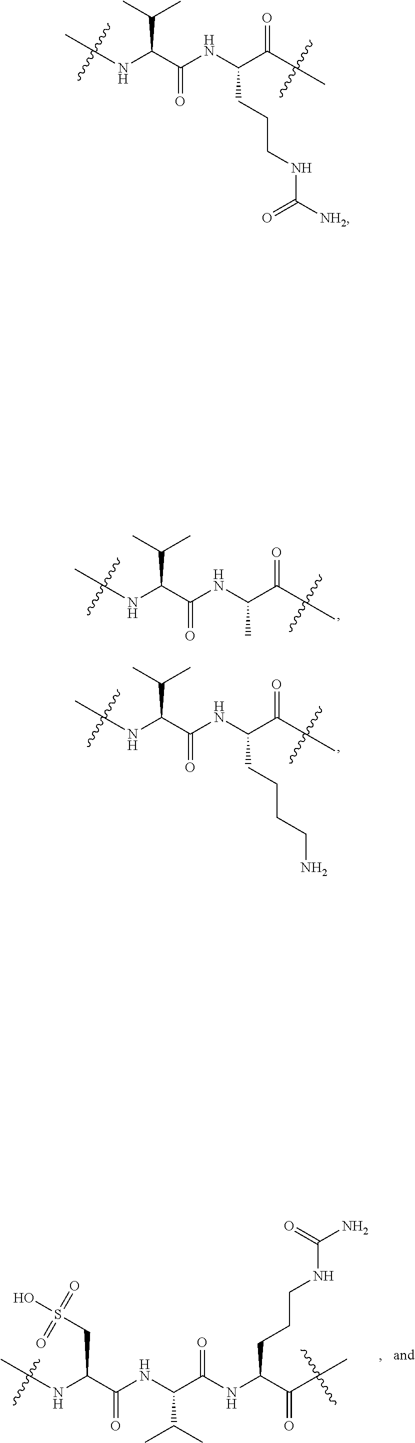

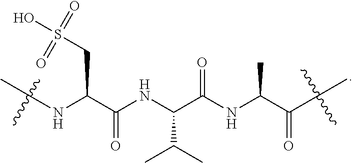 [SMILES: *N[C@@H](CS(=O)(=O)O)C(=O)N[C@H](C(=O)N[C@@H](C)C(*)=O)C(C)C]

19. The antibody-drug conjugate of claim 18, wherein (i) the cleavable group comprises a pyrophosphate and/or a self-immolative group; (ii) the cleavable group comprises a self-immolative group; or (iii) the cleavable group comprises a self-immolative group comprising para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium.

20. The antibody-drug conjugate of claim 19, wherein m is 0 or 1 or m is 1 and the bridging spacer comprises

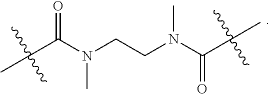 [SMILES: *C(=O)N(C)CCN(C)C(*)=O]

21. The antibody-drug conjugate of claim 20, wherein:
(1) -(L-D) is formed from a compound selected from: 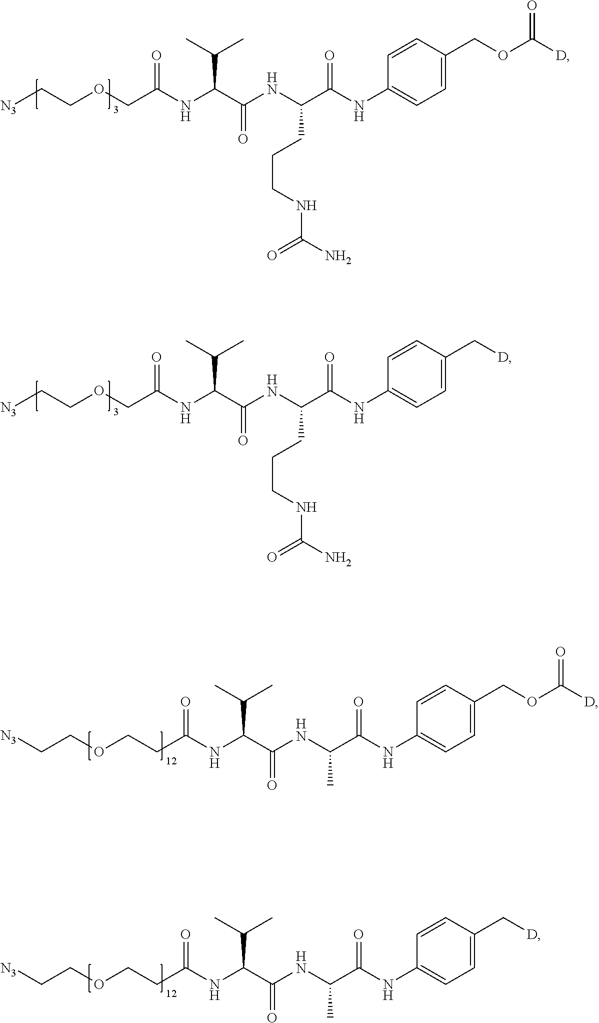 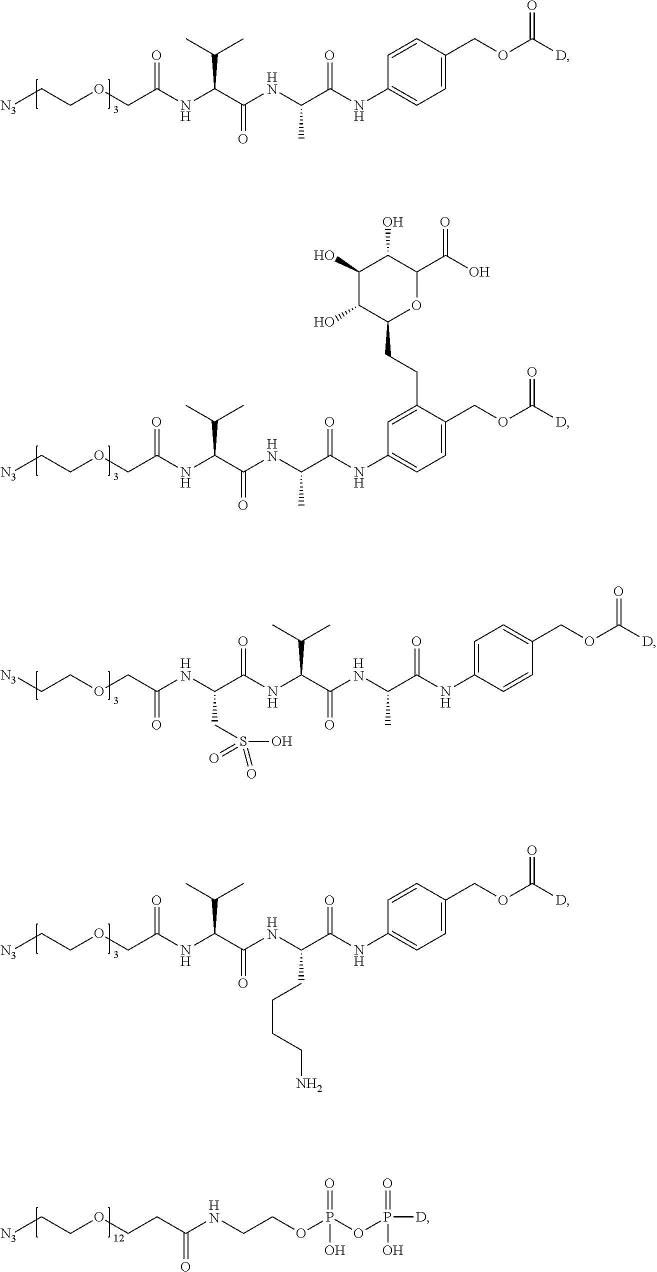 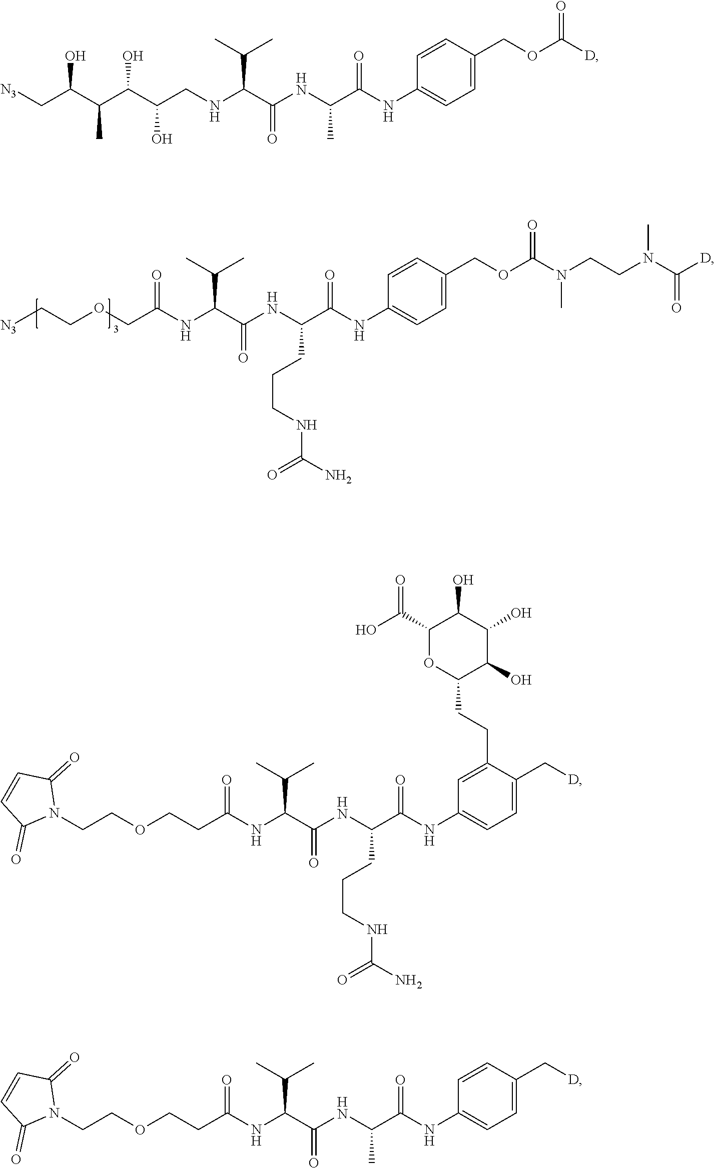 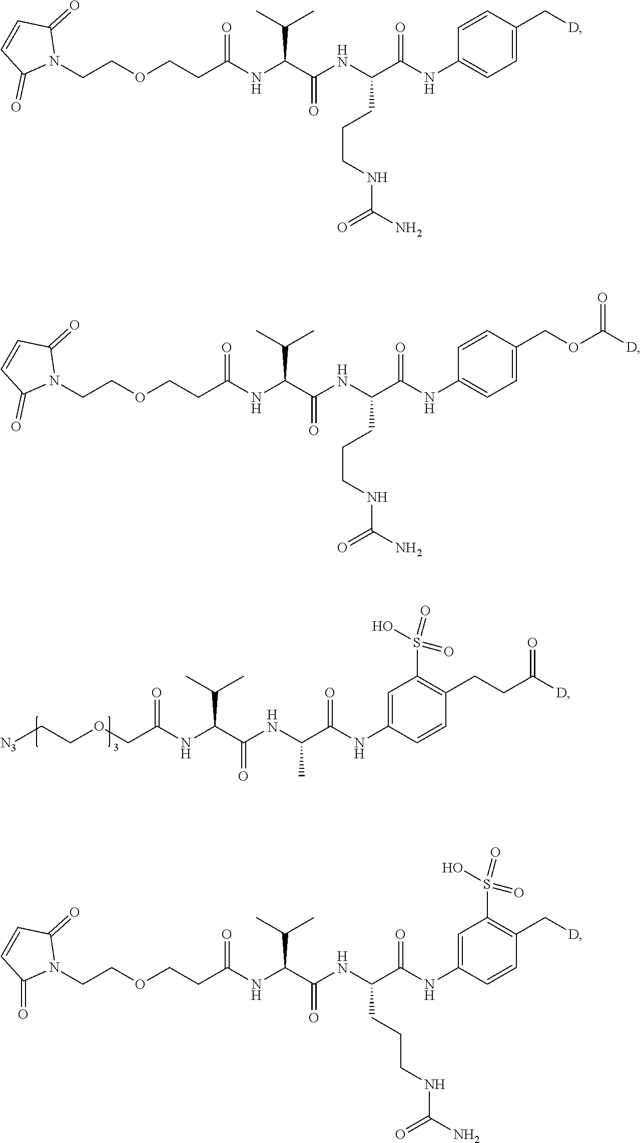 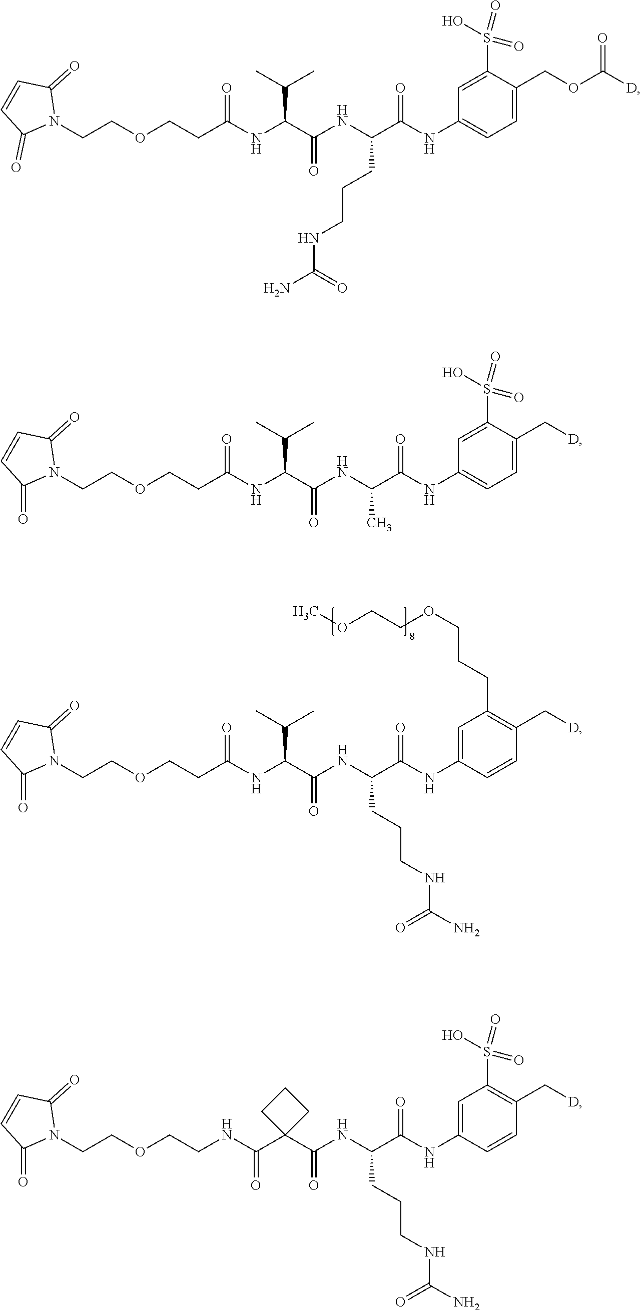 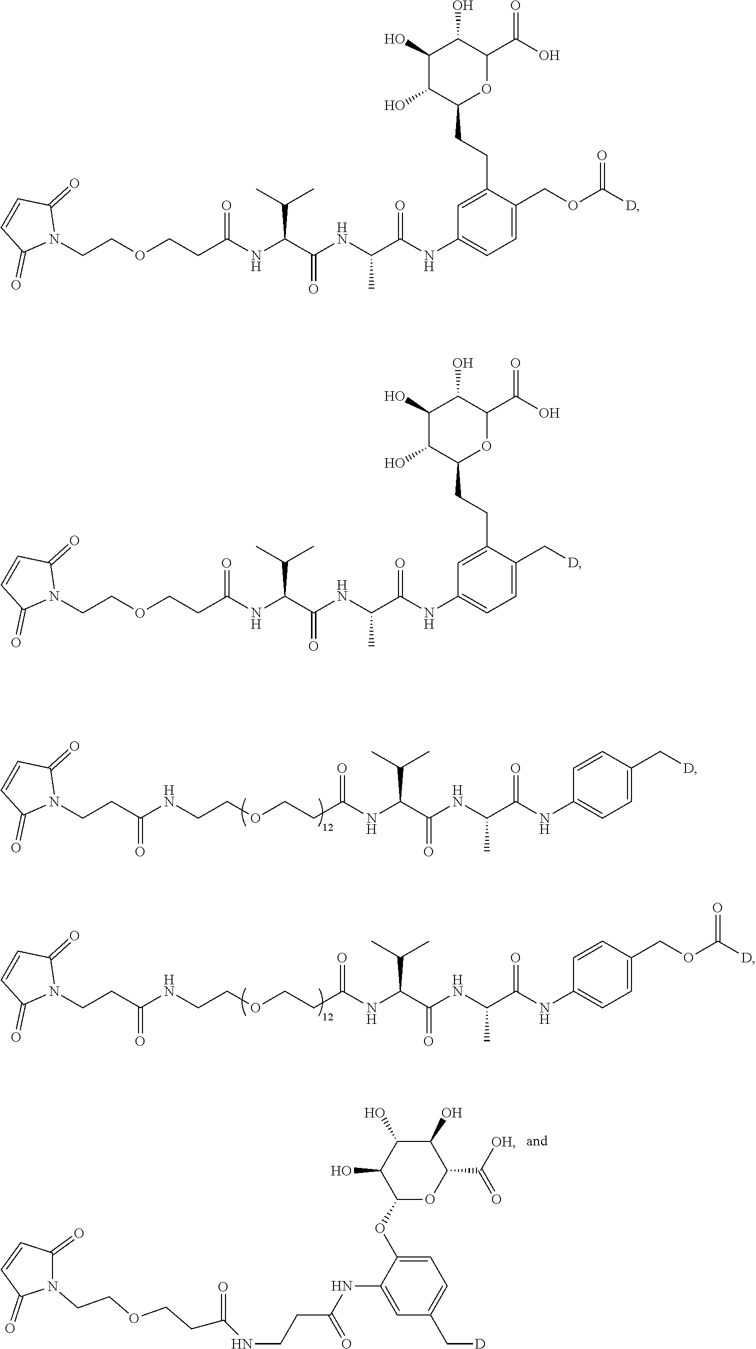 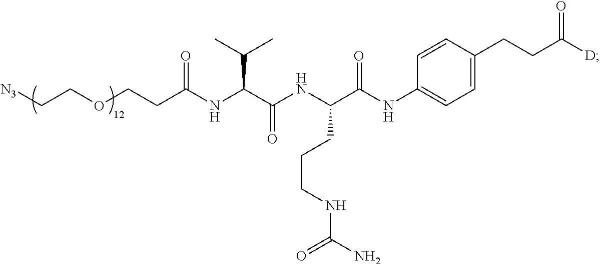
or
(2) -(L-D) comprises a formula selected from: 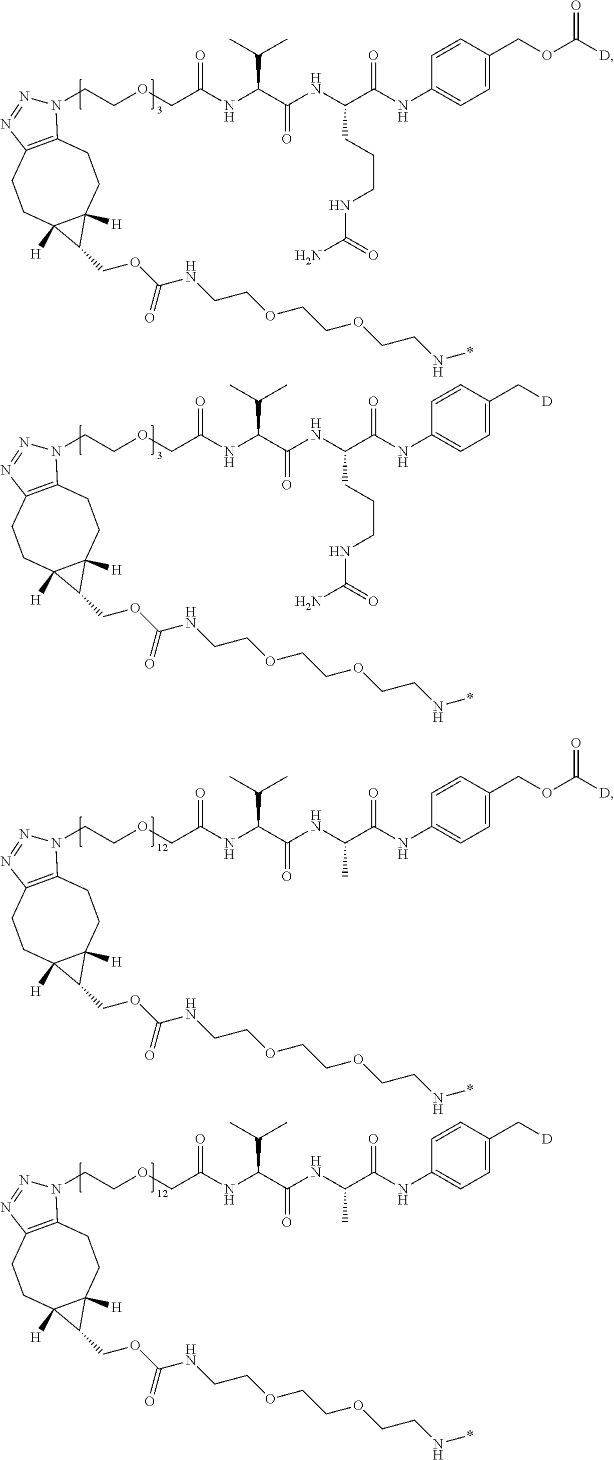 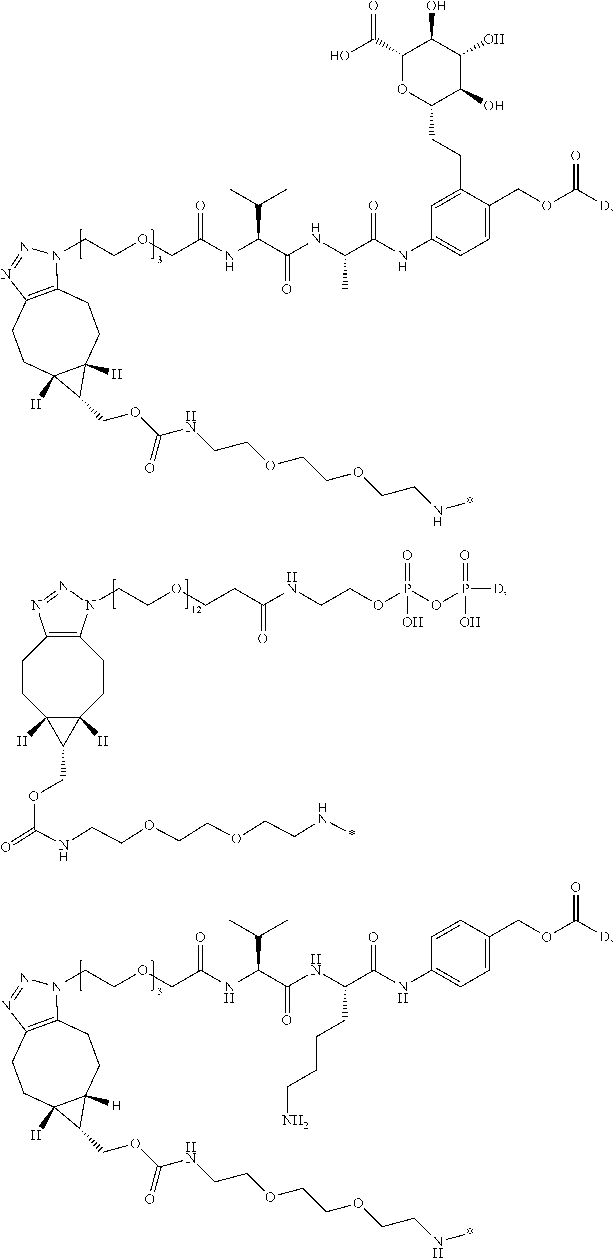 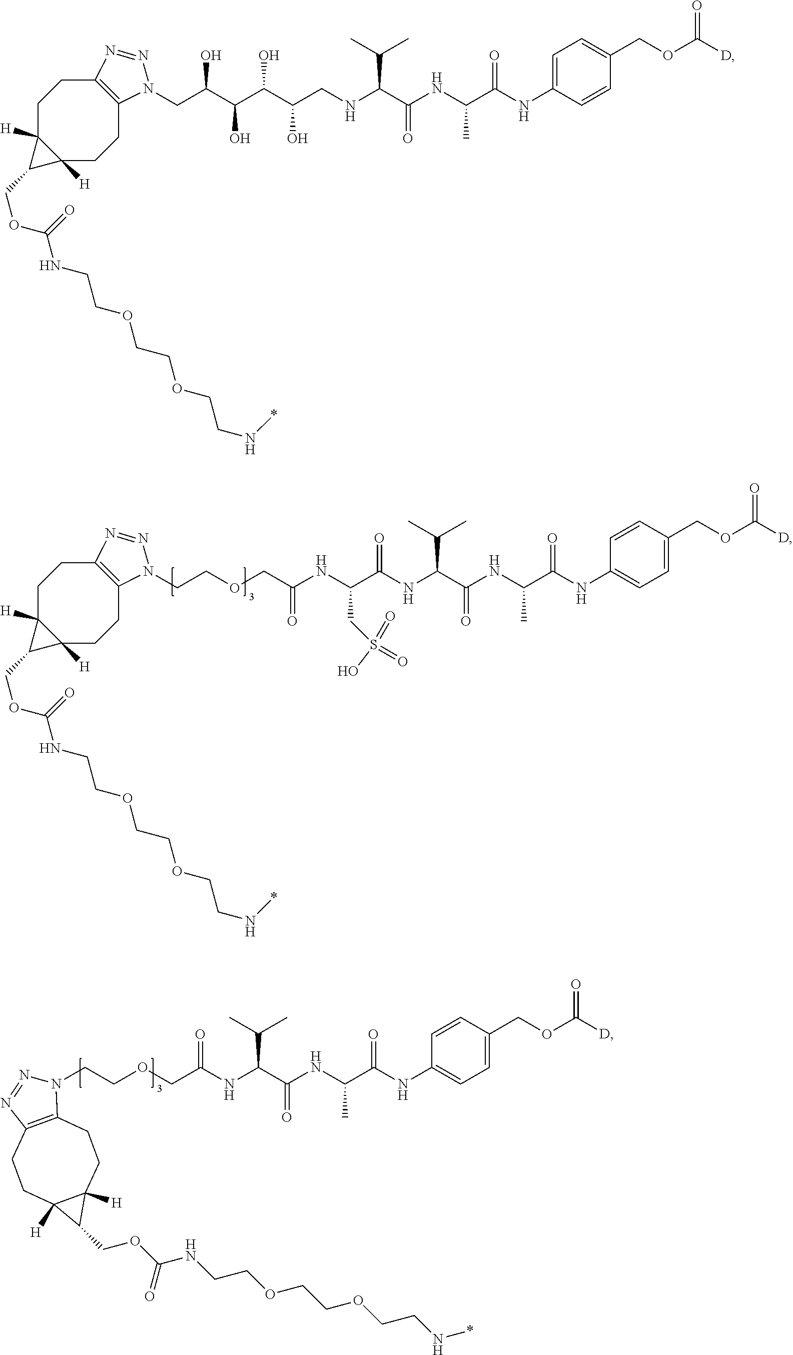 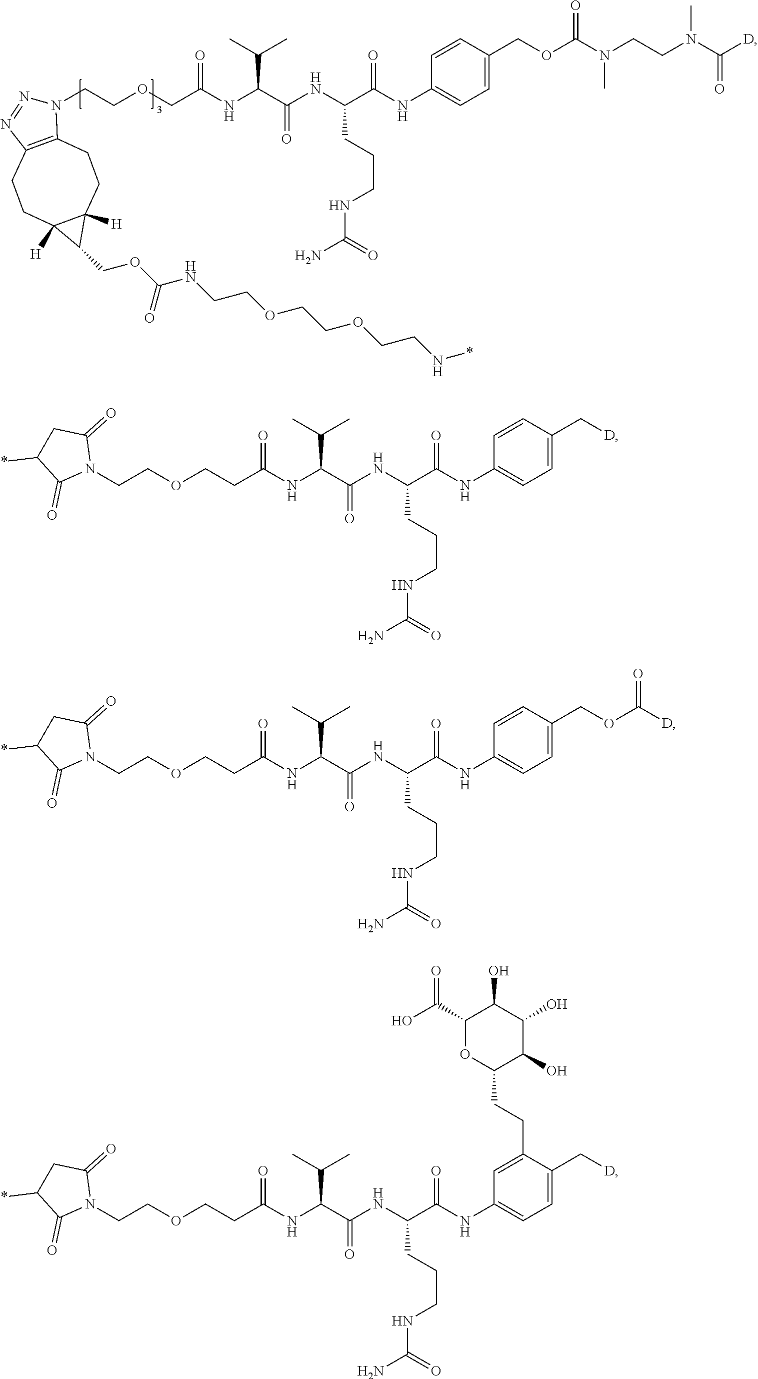 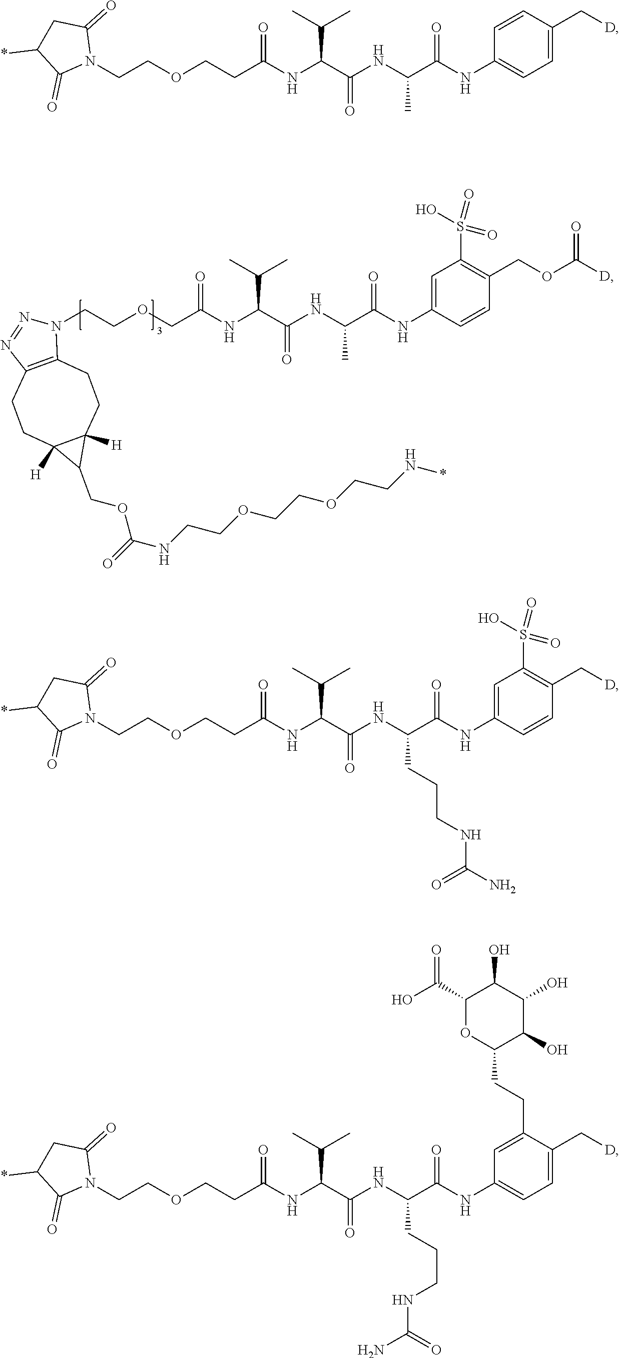 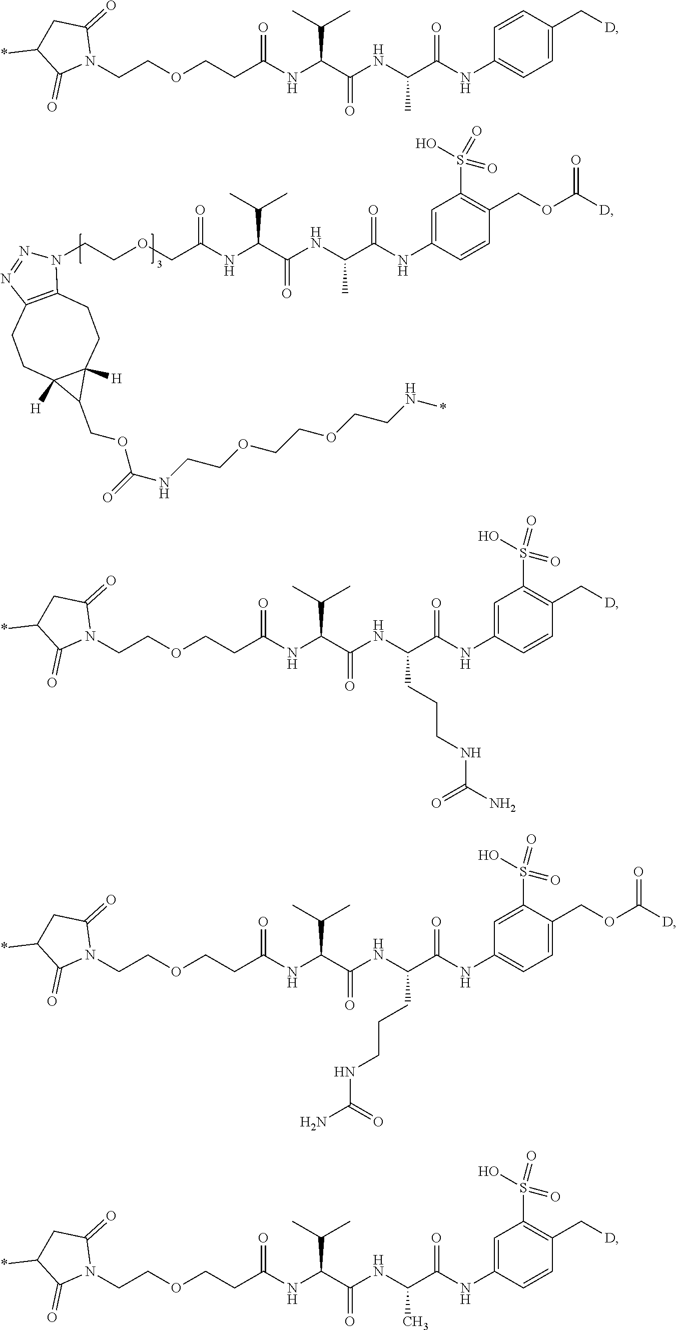 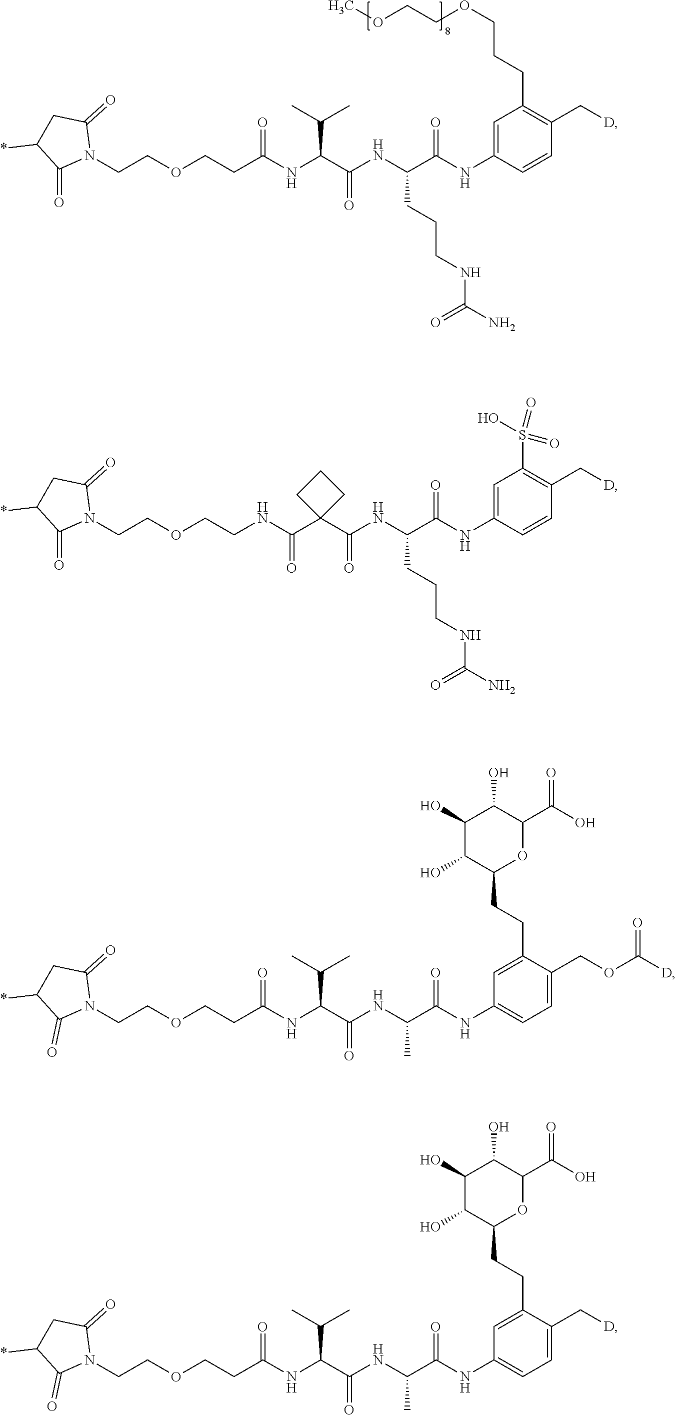 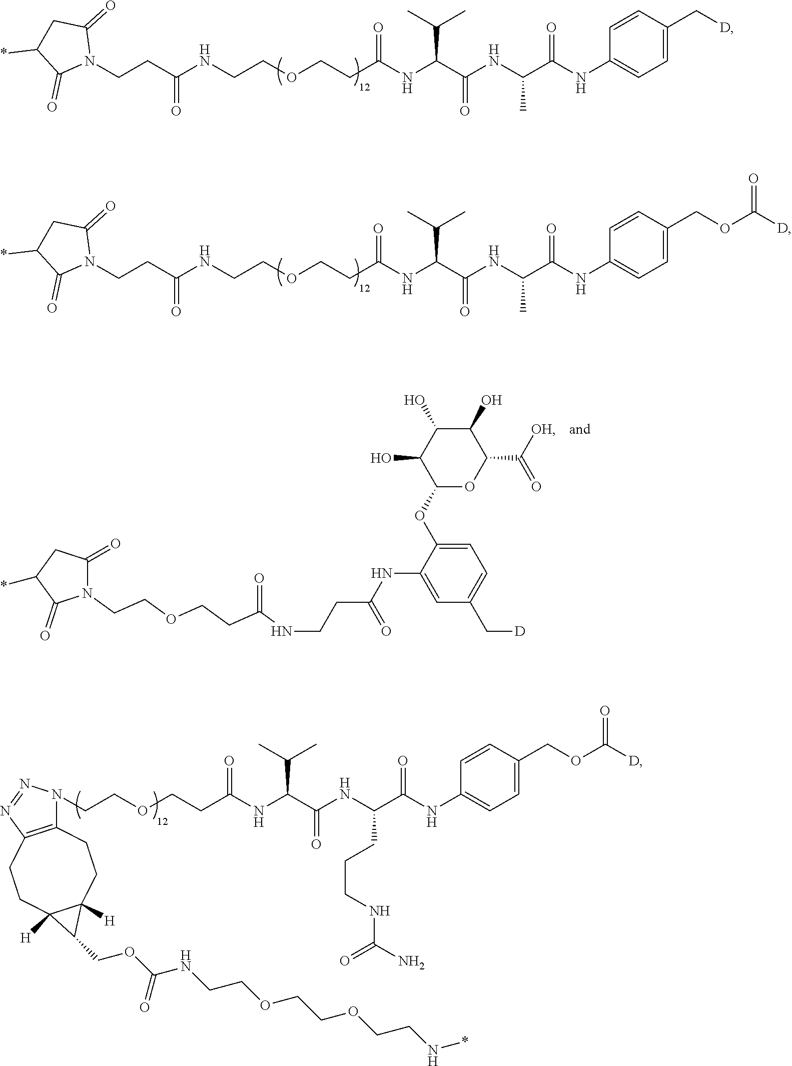
and
wherein [symbol] is a bond to the antibody.

22. (canceled)

23. The antibody-drug conjugate of claim 1, wherein;
(i)-(L-D) is of the formula (C): 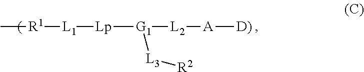
wherein:
R₁ is an attachment group;
L₁ is a bridging spacer,
Lₚ is a peptide group comprising 1 to 6 amino acids;
D is a Bcl-xL inhibitor,
G₁-L₂-A is a self-immolative spacer,
L₂ is a bond, a methylene, a neopentylene or a C₂-C₃ alkenylene;
A is a bond, —OC(═O)—*, 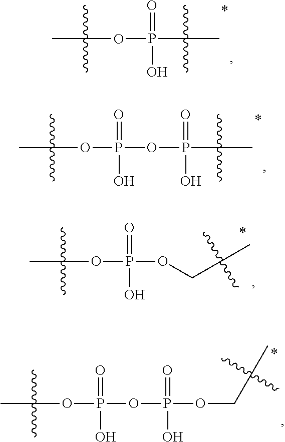 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D;
L3 is a spacer moiety; and
R₂ is a hydrophilic moiety;
(ii)-(L-D) is of Formula (D): 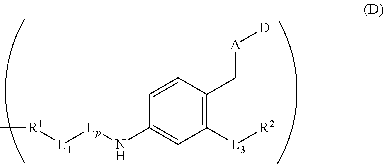
wherein:
R₁ is an attachment group;
L₁ is a bridging spacer;
Lp is a peptide group comprising 1 to 6 amino acids;
A is a bond, —OC(═O)—*, 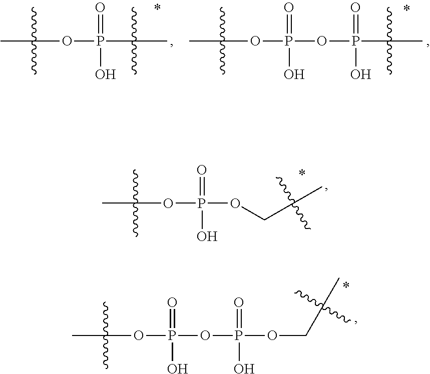 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D;
L₃ is a spacer moiety; and
R² is a hydrophilic moiety; optionally wherein:
(1) L₁ comprises: 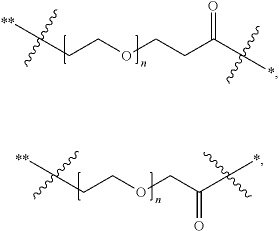
or
*—CH(OH)CH(OH)CH(OH)CH(OH)—**,
wherein each n is an integer from 1 to 12, wherein the * of L₁ indicates the point of direct or indirect attachment to Lp, and the ** of L₁ indicates the point of direct or indirect attachment to R¹;
(2) L₁ is 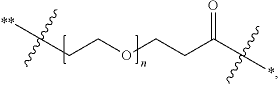
and n is an integer from 1 to 12 or n is 1 or n is 12, wherein the * of L₁ indicates the point of direct or indirect attachment to Lp, and the ** of L₁ indicates the point of direct or indirect attachment to R¹;
(3) L₁ is 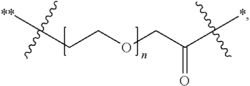
and n is an integer from 1 to 12, wherein the * of L₁ indicates the point of direct or indirect attachment to Lp, and the ** of L₁ indicates the point of direct or indirect attachment to R¹;
(4) L₁ comprises 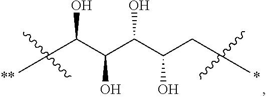 [SMILES: *C[C@H](O)[C@@H](O)[C@@H](O)[C@@H](*)O]
wherein the * of L₁ indicates the point of direct or indirect attachment to Lp, and the ** of L₁ indicates the point of direct or indirect attachment to R¹; or
(5) L₁ is a bridging spacer comprising:
*—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R³)₂(CH₂)ₘC(═O)NR³ (CH₂)NR³C(═O)(CH₂)ₘ—**; *—C(═O)O(CH₂)ₘC(═O)NH(CH₂)ₘ—**; *—C(═O)(CH₂)NH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—*; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁ (CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, wherein the * of L₁ indicates the point of direct or indirect attachment to Lp, and the ** of L₁ indicates the point of direct or indirect attachment to R¹;
X₁ is 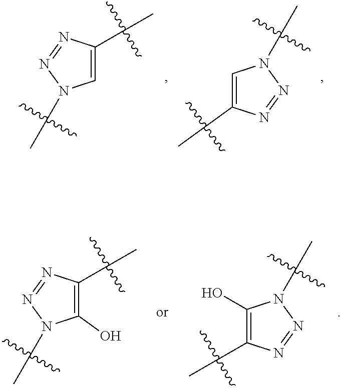 [SMILES: *c1cn(*)nn1 ; *c1cn(*)nn1 ; *c1nnn(*)c1O ; *c1nnn(*)c1O]
and
each m is independently selected from 1, 2, 3, 4, 5, 6, 7, 8, 9 and 10;
each n is independently selected from 1, 2, 3, 4, 5, 6, 7, 8, 9 and 10; and
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
and each R³ is independently selected from H and C₁-C₆alkyl.

24-25. (canceled)

26. The antibody-drug conjugate of claim 23, wherein:
(1) R² is a hydrophilic moiety comprising polyethylene glycol, polyalkylene glycol, a polyol, a polysarcosine, a sugar, an oligosaccharide, a polypeptide, C₂-C₆ alkyl substituted with 1 to 3 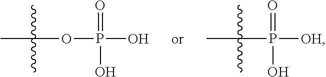 [SMILES: *OP(=O)(O)O ; *P(=O)(O)O]
or C₂-C₆alkyl substituted with 1 to 2 substituents independently selected from —OC(═O)NHS(O)₂NHCH₂CH₂OCH₃, —NHC(═O)C₁₋₄alkylene-P(O)(OCH₂CH₃)₂ and —COOH groups:
(2) R₂ is 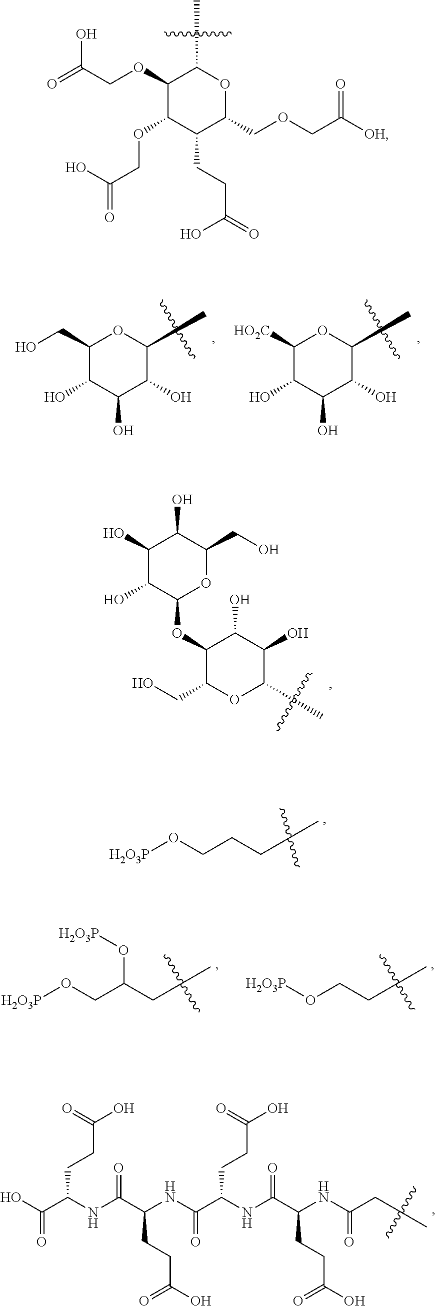 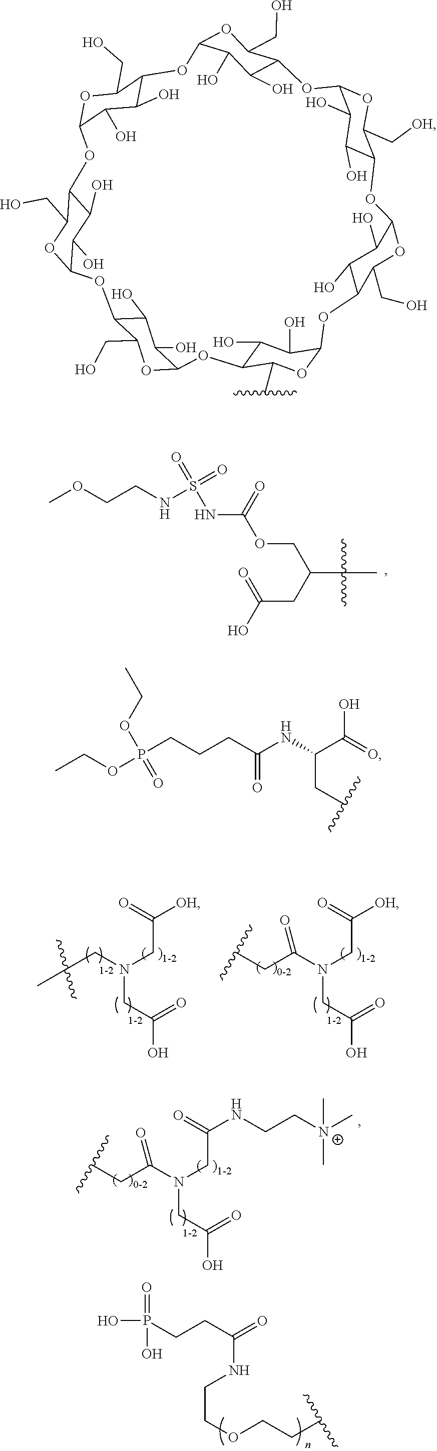
wherein
n is an integer between 1 and 6, 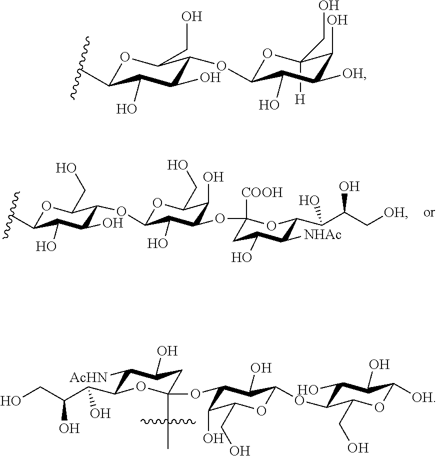
(3) the hydrophilic moiety represented by R² comprises;
(i) a polysarcosine with the following moiety; 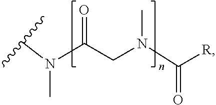
wherein n is an integer between 3 and 25; and R is H, —CH₃ or —CH₂CH₂C(═O)OH; or
(ii) a polyethylene glycol of formula: 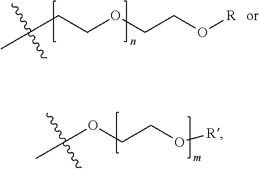
wherein R is H, —CH₃, CH₂CH₂NHC(═O)ORₐ, —CH₂CH₂NHC(═O)Rₐ, or —CH₂CH₂C(═O)ORₐ, R′ is OH, —OCH₃, —CH₂CH₂NHC(═O)ORₐ, —CH₂CH₂NHC(═O)Rₐ, or —OCH₂CH₂C(═O)ORₐ, in which Rₐ is H or C₁₋₄ alkyl optionally substituted with either OH or C₁₋₄ alkoxyl, and each of m and n is independently an integer between 2 and 25; or (4) the hydrophilic moiety represented by R² comprises

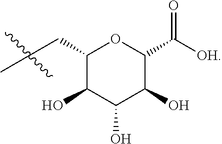

27-29. (canceled)

30. The antibody-drug conjugate of claim 26, wherein:
(i) L₃ is a spacer moiety having the structure 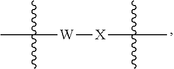
wherein:
W is —CH₂—, —CH₂O—, —CH₂N(R^{b})C(═O)O—, —NHC(═O)C(R^{b})₂NHC(═O)O—, —NHC(═O)C(R^{b})₂NH—, —NHC(═O)C(R^{b})₂NHC(═O)—, —CH₂N(X—R²)C(═O)O—, —C(═O)N(X—R²)—, —CH₂N(X—R²)C(═O)—, —C(═O)NR^{b}—, —C(═O)NH—, —CH₂NR^{b}C(═O)—, —CH₂NR^{b}C(═O)NH—, —CH₂NR^{b}C(═O)NR^{b}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{b} is independently selected from H, C₁-C₆alkyl, and C₃-C₈ cycloalkyl; and
X is a bond, triazolyl, or —CH₂-triazolyl-,
wherein X is connected to R²; or
(ii) L₃ is a spacer moiety having the structure 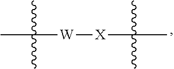
wherein:
W is —CH₂—, —CH₂O—, —CH₂N(R^{b})C(═O)O—, —NHC(═O)C(R^{b})₂NHC(═O)O—, —NHC(═O)C(R^{b})₂NH—, —NHC(═O)C(R^{b})₂NHC(═O)—, —CH₂N(X—R²)C(═O)O—, —C(═O)N(X—R²)—, —CH₂N(X—R²)C(═O)—, —C(═O)NR^{b}—, —C(═O)NH—, —CH₂NR^{b}C(═O)—, —CH₂NR^{b}C(═O)NH—, —CH₂NR^{b}C(═O)NR^{b}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{b} is independently selected from H, C₁-C₆alkyl, and C₃-C₈ cycloalkyl; and
X is —CH₂-triazolyl-C₁₋₄ alkylene-OC(O)NHS(O)₂NH—, —C₄₋₆ cycloalkylene-OC(O)NHS(O)₂NH—, —(CH₂CH₂O), —C(O)NHS(O)₂NH—, —(CH₂CH₂O)ₙ—C(O)NHS(O)₂NH—(CH₂CH₂O)ₙ—, —CH₂-triazolyl-C₁₋₄ alkylene-OC(O)NHS(O)₂NH—(CH₂CH₂O)ₙ—, —C₄₋₆cycloalkylene-OC(O)NHS(O)₂NH—(CH₂CH₂O)ₙ—, wherein each n independently is 1, 2, or 3,
wherein X is connected to R².

31. The antibody-drug conjugate of claim 30, wherein:
(1) the attachment group is formed by a reaction comprising at least one reactive group;
(2) the attachment group is formed by reacting:
a first reactive group that is attached to the linker, and
a second reactive group that is attached to the antibody or antigen-binding fragment or is an amino acid residue of the antibody or antigen-binding fragment, wherein optionally,
(i) at least one of the reactive groups comprises:
a thiol,
a maleimide,
a haloacetamide,
an azide,
an alkyne,
a cyclooctene,
a triaryl phosphine,
an oxanorbornadiene,
a cyclooctyne,
a diaryl tetrazine,
a monoaryl tetrazine,
a norbornene,
an aldehyde,
a hydroxylamine,
a hydrazine,
NH₂—NH—C(═O)—,
a ketone,
a vinyl sulfone,
an aziridine,
an amino acid residue, 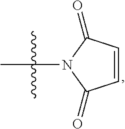 [SMILES: *N1C(=O)C=CC1=O]
—ONH₂, —NH₂, 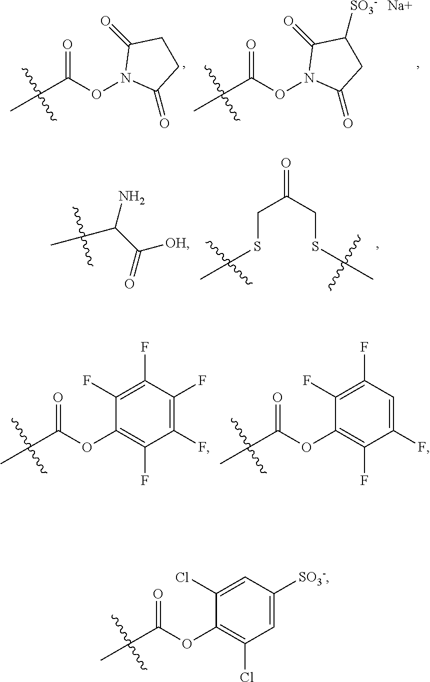
—N₃, 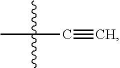 [SMILES: *C#C]
—SH, —SR₃, —SSR₄, —S(═O)₂(CH═CH₂), —(CH₂)₂S(═O)₂(CH═CH₂), —NHS(═O)₂(CH═CH₂), —NHC(═O)CH₂Br, —NHC(═O)CH₂I, 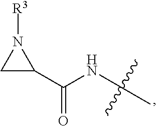
—C(O)NHNH₂, 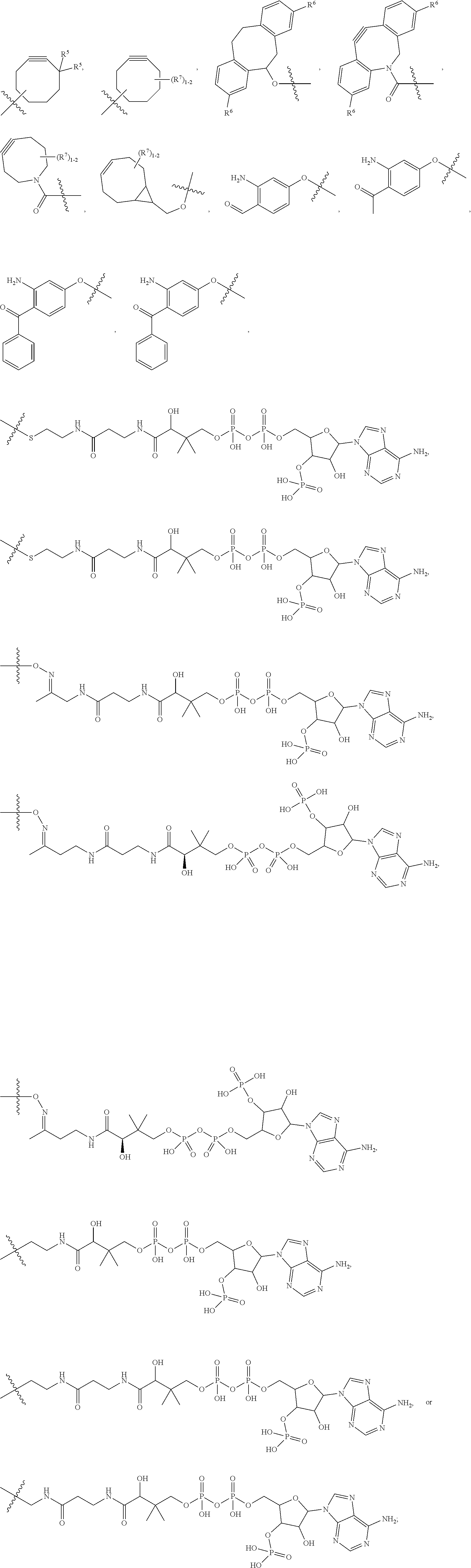
wherein:
each R³ is independently selected from H and C₁-C₆alkyl;
each R⁴ is 2-pyridyl or 4-pyridyl;
each R⁵ is independently selected from H, C₁-C₆alkyl, F, Cl, and —OH;
each R⁶ is independently selected from H, C₁-C₆alkyl, F, Cl, —NH₂, —OCH₃, —OCH₂CH₃, —N(CH₃)₂, —CN, —NO₂ and —OH;
each R⁷ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH; and/or
(ii) the first reactive group and second reactive group comprise:
a thiol and a maleimide,
a thiol and a haloacetamide,
a thiol and a vinyl sulfone,
a thiol and an aziridine,
an azide and an alkyne,
an azide and a cyclooctyne,
an azide and a cyclooctene,
an azide and a triaryl phosphine,
an azide and an oxanorbornadiene,
a diaryl tetrazine and a cyclooctene,
a monoaryl tetrazine and a norbornene,
an aldehyde and a hydroxylamine,
an aldehyde and a hydrazine,
an aldehyde and NH₂—NH—C(═O)—,
a ketone and a hydroxylamine,
a ketone and a hydrazine,
a ketone and NH₂—NH—C(═O)—,
a hydroxylamine and 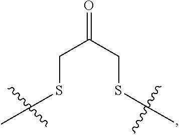
an amine and 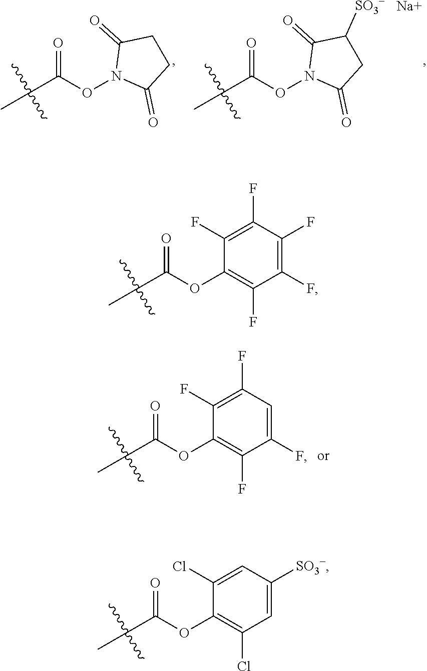
or
a CoA or CoA analogue and a serine residue; and/or
(3) the attachment group comprises a group selected from: 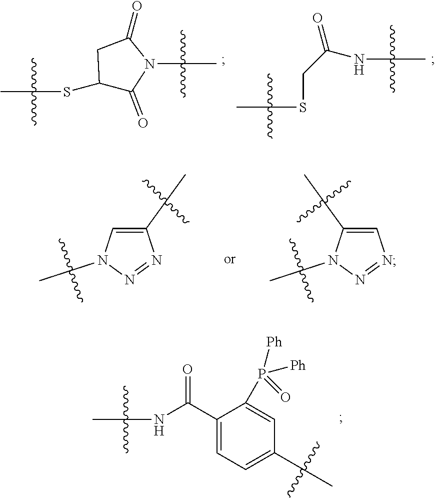 [SMILES: *SC1CC(=O)N(*)C1=O ; *NC(=O)CS* ; *c1cn(*)nn1 ; "or" ; *c1cnnn1* ; *NC(=O)c1ccc(*)cc1P(=O)(c1ccccc1)c1ccccc1] 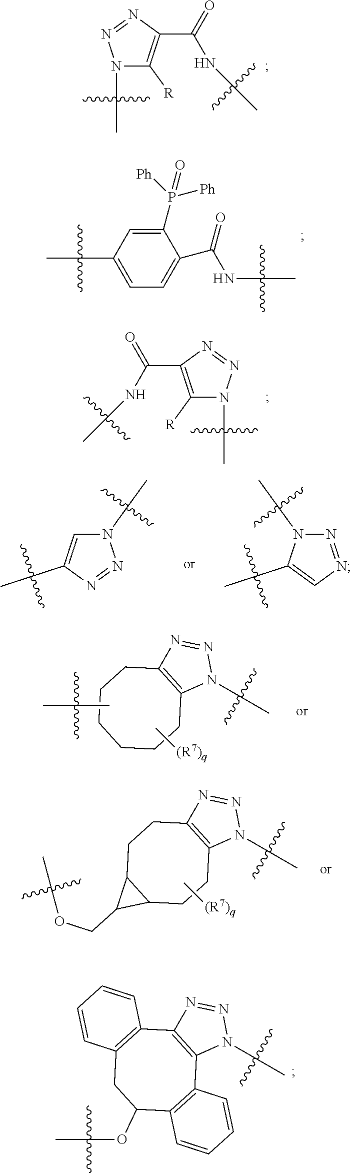 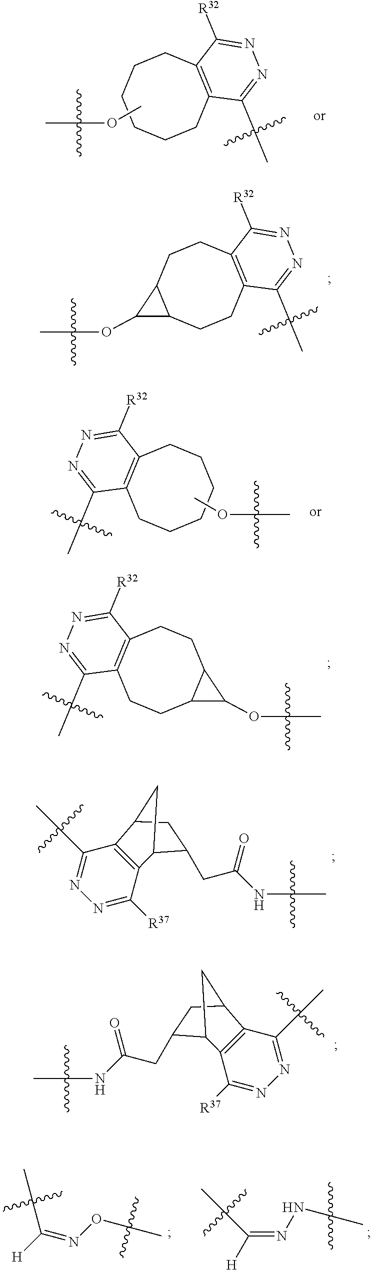 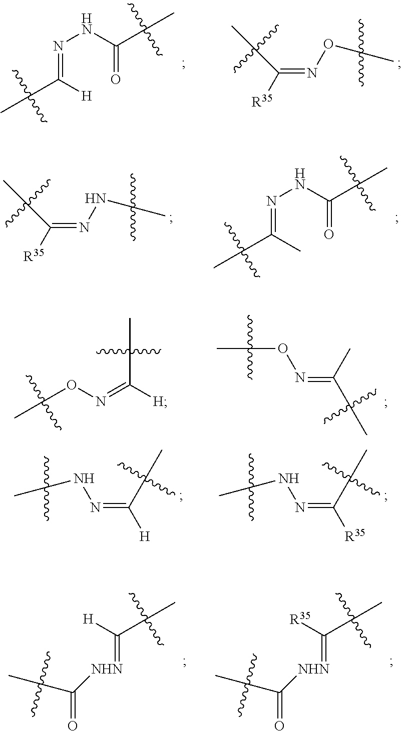 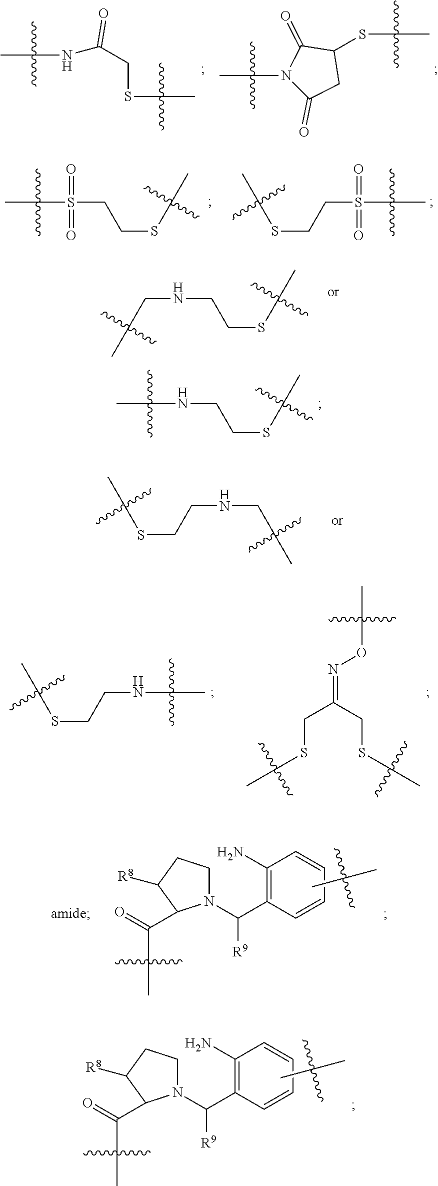 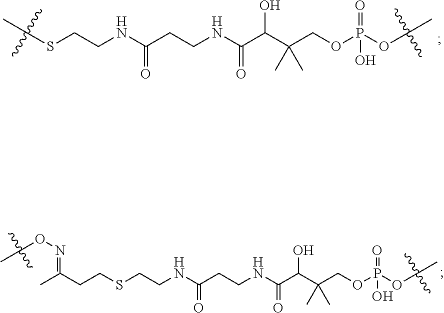 [SMILES: *OP(=O)(O)OCC(C)(C)C(O)C(=O)NCCC(=O)NCCS* ; *O/N=C(\C)CCSCCNC(=O)CCNC(=O)C(O)C(C)(C)COP(=O)(O)O*] 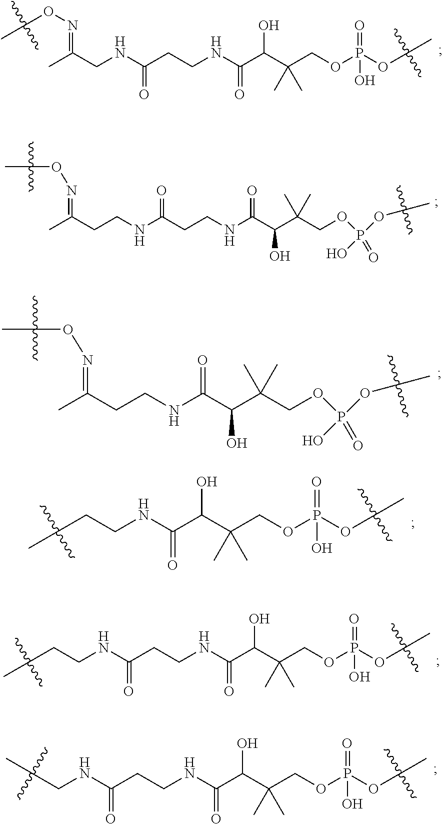 [SMILES: *O/N=C(\C)CNC(=O)CCNC(=O)C(O)C(C)(C)COP(=O)(O)O* ; *O/N=C(\C)CCNC(=O)CCNC(=O)[C@H](O)C(C)(C)COP(=O)(O)O* ; *O/N=C(\C)CCNC(=O)[C@H](O)C(C)(C)COP(=O)(O)O* ; *CCNC(=O)C(O)C(C)(C)COP(=O)(O)O* ; *CCNC(=O)CCNC(=O)C(O)C(C)(C)COP(=O)(O)O* ; *CNC(=O)CCNC(=O)C(O)C(C)(C)COP(=O)(O)O*]
and
disulfide,
wherein:
R³² is H, C₁₋₄ alkyl, phenyl, pyrimidine or pyridine;
R³⁵ is H, C₁₋₆ alkyl, phenyl or C₁₋₄ alkyl substituted with 1 to 3 —OH groups;
each R⁷ is independently selected from H, C₁₋₆ alkyl, fluoro, benzyloxy substituted with —C(═O)OH, benzyl substituted with —C(═O)OH, C₁₋₄ alkoxy substituted with —C(═O)OH and C₁₋₄ alkyl substituted with —C(═O)OH;
R³⁷ is independently selected from H, phenyl and pyridine;
q is 0, 1, 2 or 3;
R⁸ is H or methyl; and
R⁹ is H, —CH₃ or phenyl.

32-33. (canceled)

34. The antibody-drug conjugate of claim 23, wherein:
(1) the peptide group represented by Lp comprises 1 to 4 or 1 to 3 or 1 or 2 amino acid residues, optionally the amino acid residues are selected from glycine (Gly), L-valine (Val), L-citrulline (Cit), L-cysteic acid (sulfo-Ala), L-lysine (Lys), L-isoleucine (Ile), L-phenylalanine (Phe), L-methionine (Met), L-asparagine (Asn), L-proline (Pro), L-alanine (Ala), L-leucine (Leu), L-tryptophan (Trp), and L-tyrosine (Tyr);
(2) the peptide group represented by Lp comprises Val-Cit, Phe-Lys, Val-Ala, Val-Lys, Leu-Cit, sulfo-Ala-Val-Cit, sulfo-Ala-Val-Ala, Gly-Gly-Gly, and/or Gly-Gly-Phe-Gly (SEQ ID NO: 78); or
(3) the peptide group represented by Lp is selected from:

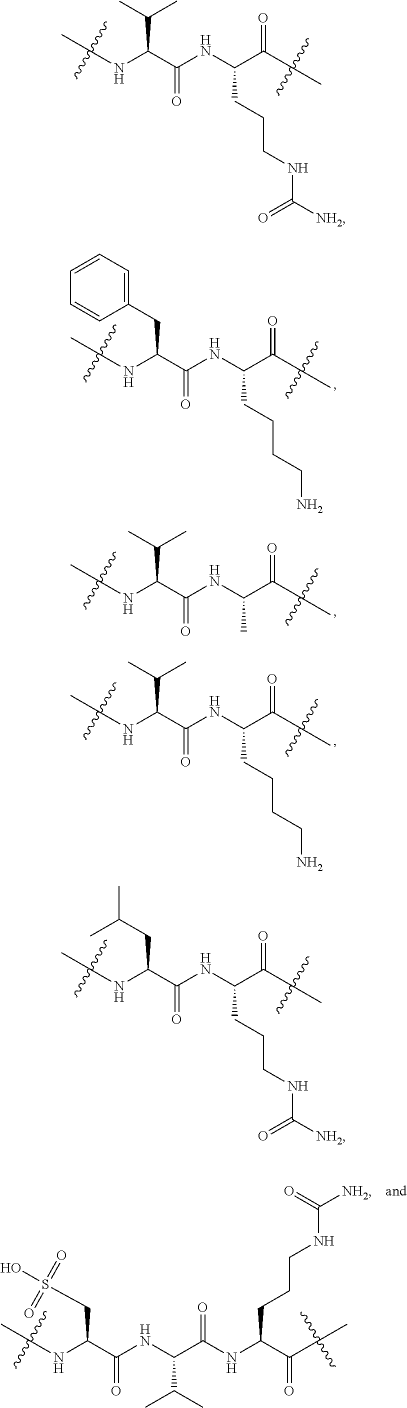

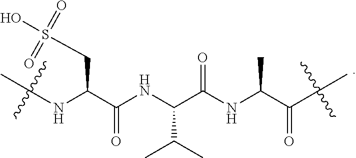 [SMILES: *N[C@@H](CS(=O)(=O)O)C(=O)N[C@H](C(=O)N[C@@H](C)C(*)=O)C(C)C]

35-36. (canceled)

37. The antibody-drug conjugate of claim 23, wherein:
-(L-D) comprises or is formed from a compound of formula: 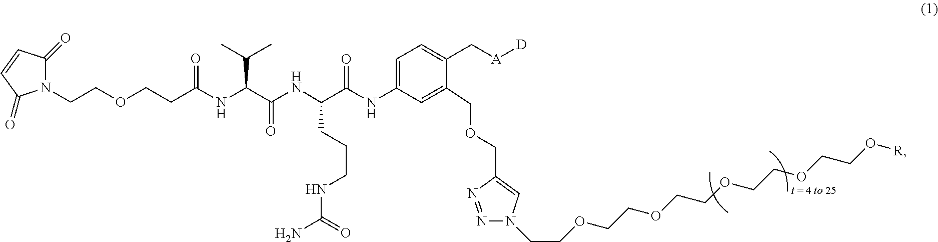
wherein:
R is H, —CH₃ or —CH₂CH₂C(═O)OH;
A is a bond, —OC(═O)—*, 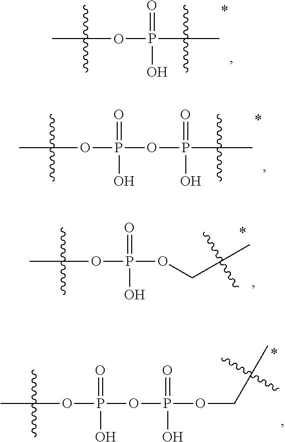 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 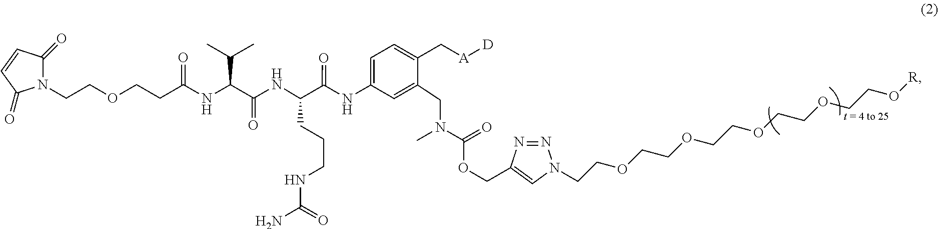
wherein:
R is H, —CH₃ or —CH₂CH₂C(═O)OH;
A is a bond, —OC(═O)—*, 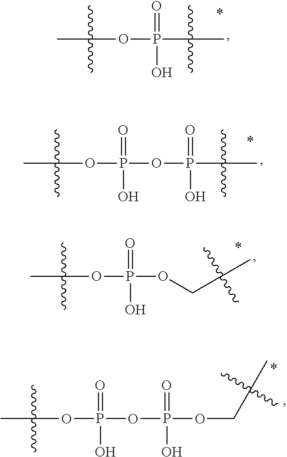 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 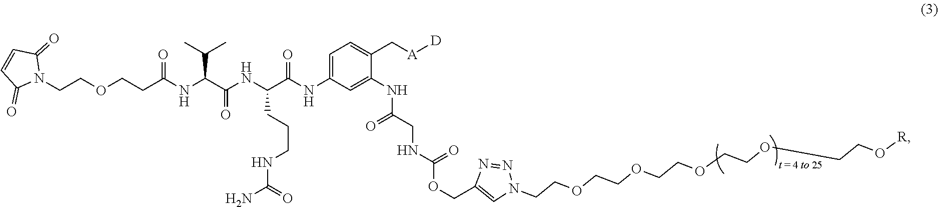
wherein:
R is H, —CH₃ or —CH₂CH₂C(═O)OH;
A is a bond, —OC(═O)—*, 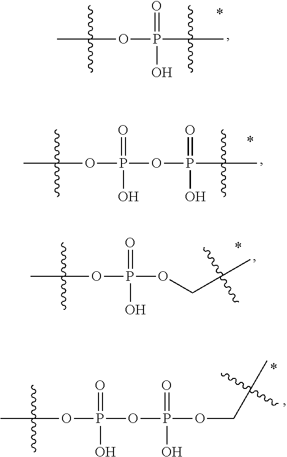 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 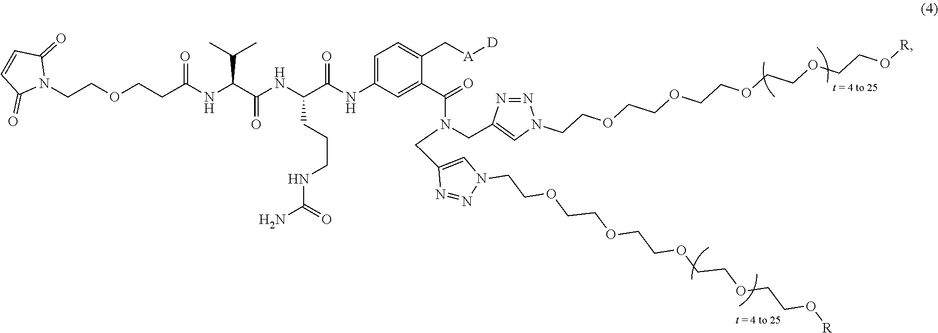
wherein:
each R is independently selected from H, —CH₃, and —CH₂CH₂C(═O)OH;
A is a bond, —OC(═O)—*, 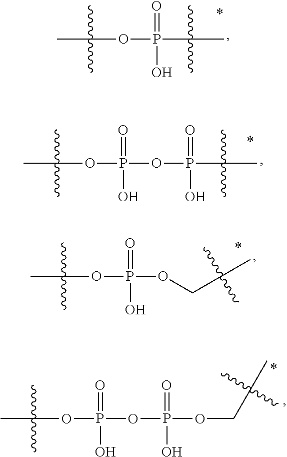 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 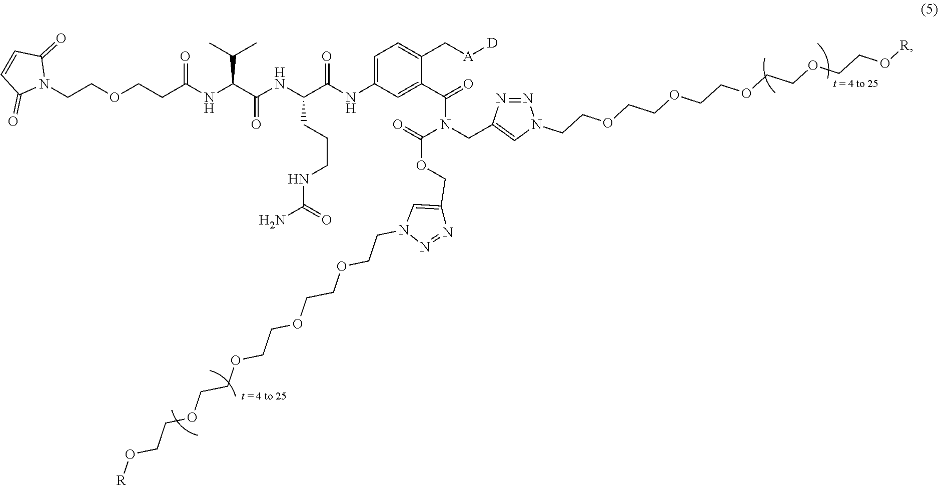
wherein:
each R is independently selected from H, —CH₃, and —CH₂CH₂C(═O)OH;
A is a bond, —OC(═O)—*, 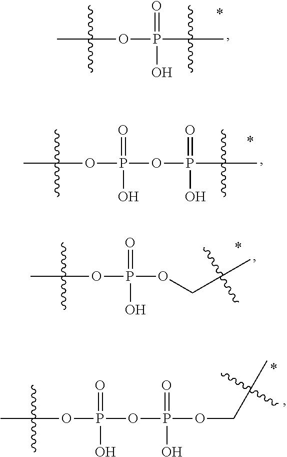 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 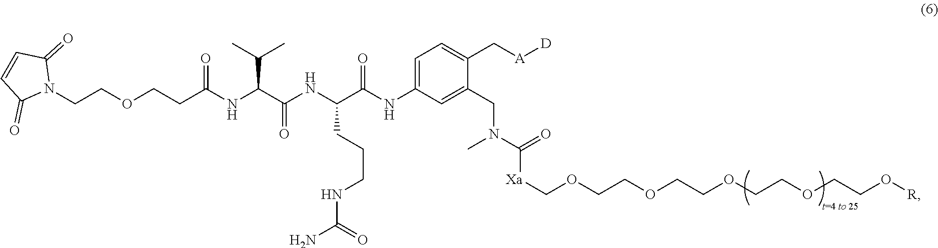
wherein:
Xa is —CH₂—, —OCH₂—, —NHCH₂— or —NRCH₂— and each R independently is H, —CH₃ or —CH₂CH₂C(═O)OH;
A is a bond, —OC(═O)—*, 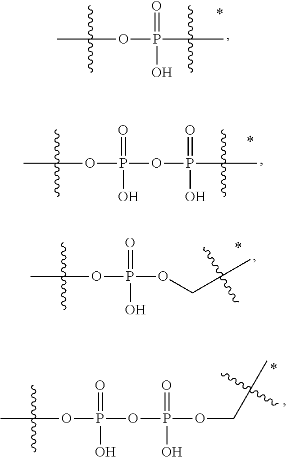 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 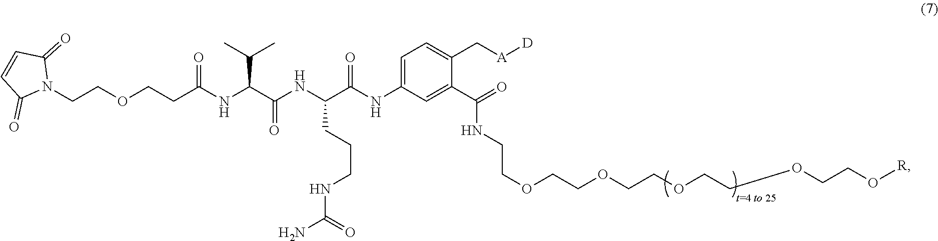
wherein:
R is H, —CH₃ or —CH₂CH₂C(═O)OH;
A is a bond, —OC(═O)—*, 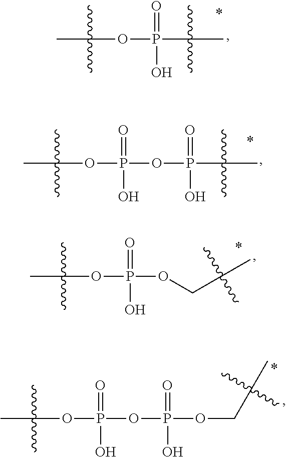 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 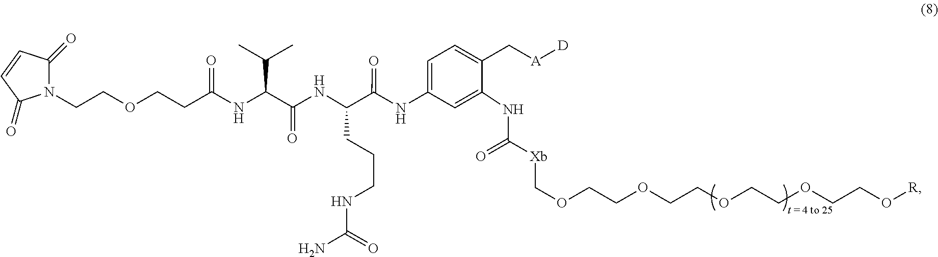
wherein:
Xb is —CH₂—, —OCH₂—, —NHCH₂— or —NRCH₂— and each R independently is H, —CH₃ or —CH₂CH₂C(═O)OH;
A is a bond, —OC(═O)—*, 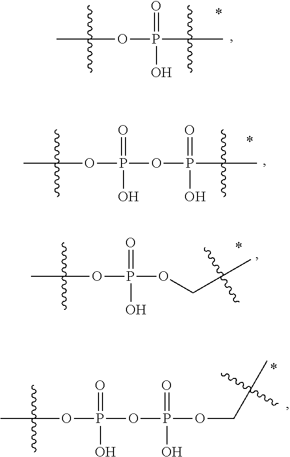 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 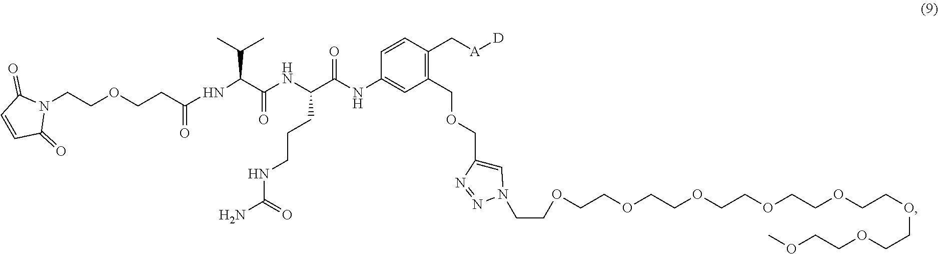
wherein:
A is a bond, —OC(═O)—*, 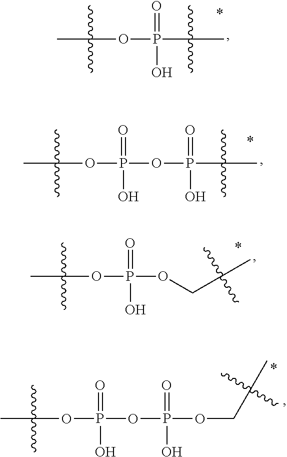 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 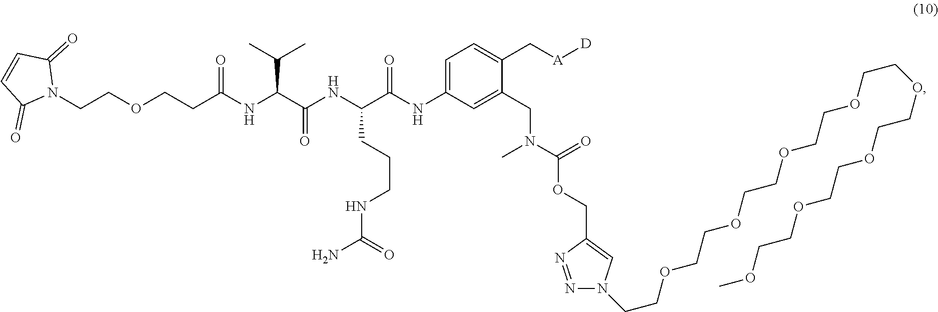
wherein:
A is a bond, —OC(═O)—*, 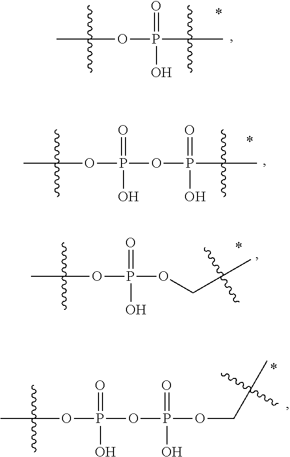 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 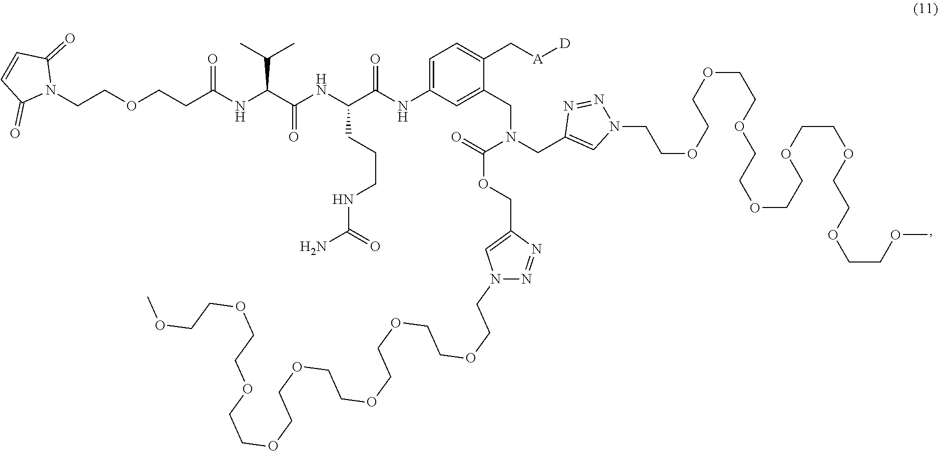
wherein:
A is a bond, —OC(═O)—*, 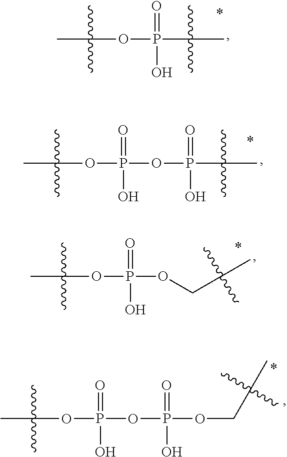 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 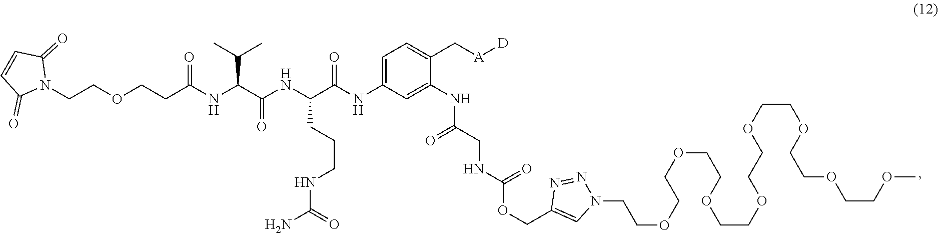
wherein:
A is a bond, —OC(═O)—*, 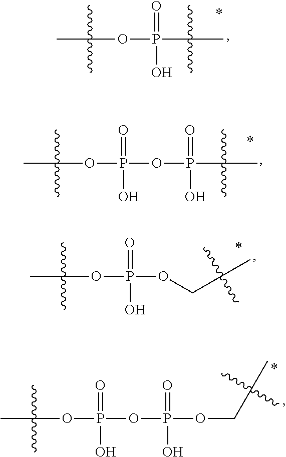 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 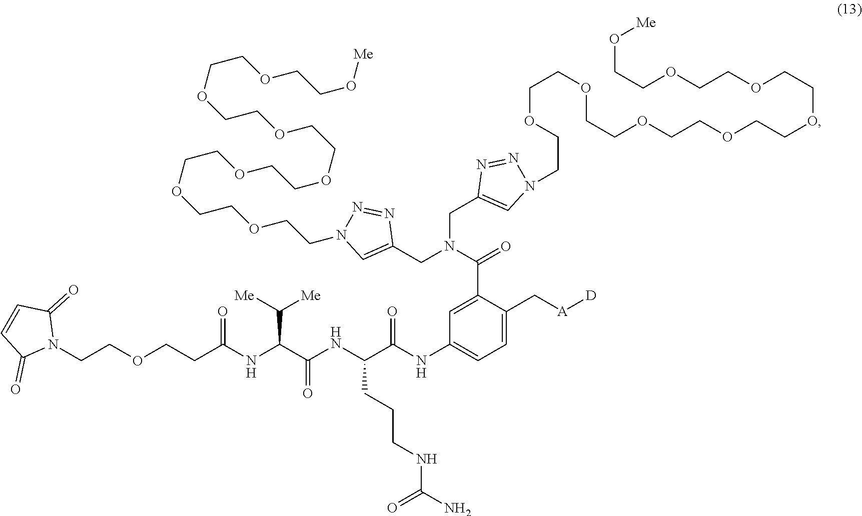
wherein:
A is a bond, —OC(═O)—*, 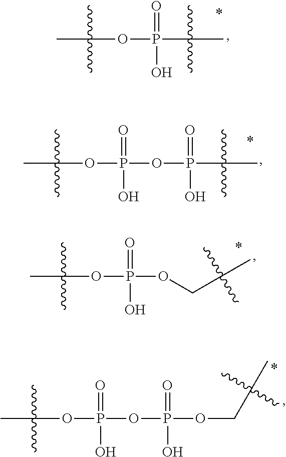 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 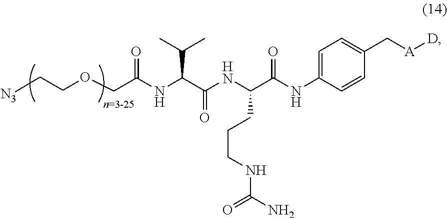
wherein:
A is a bond, —OC(═O)—*, 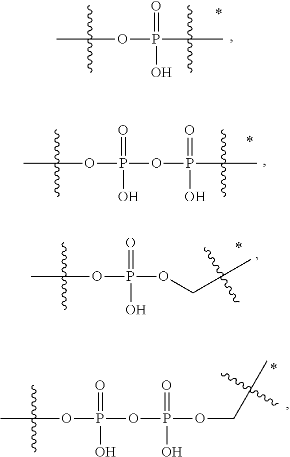 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; 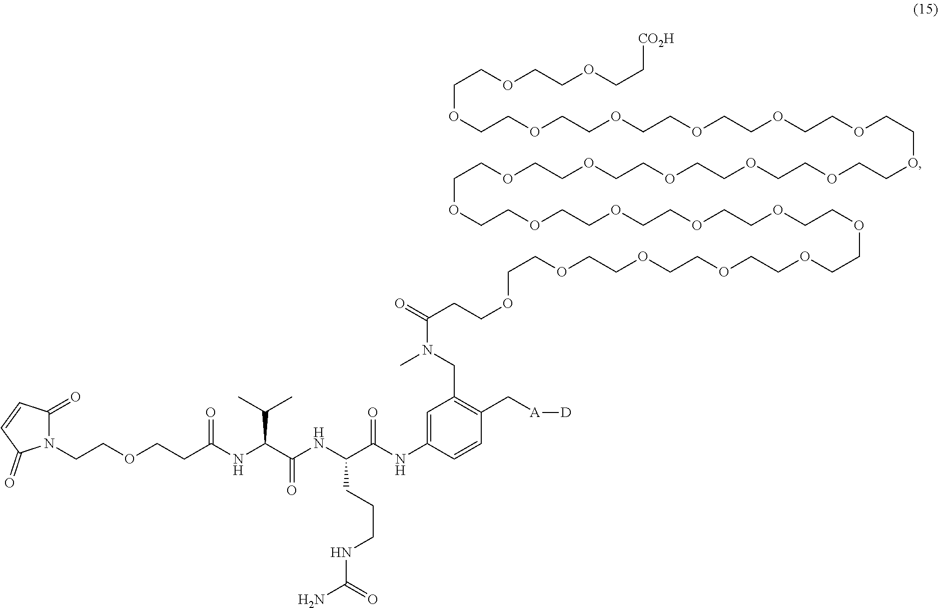
wherein:
A is a bond, —OC(═O)—*, 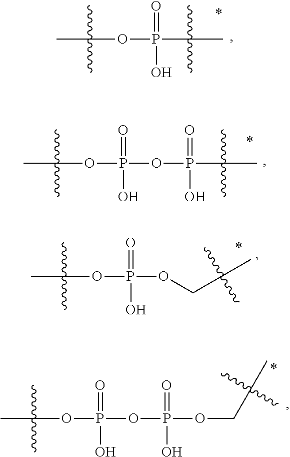 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; or 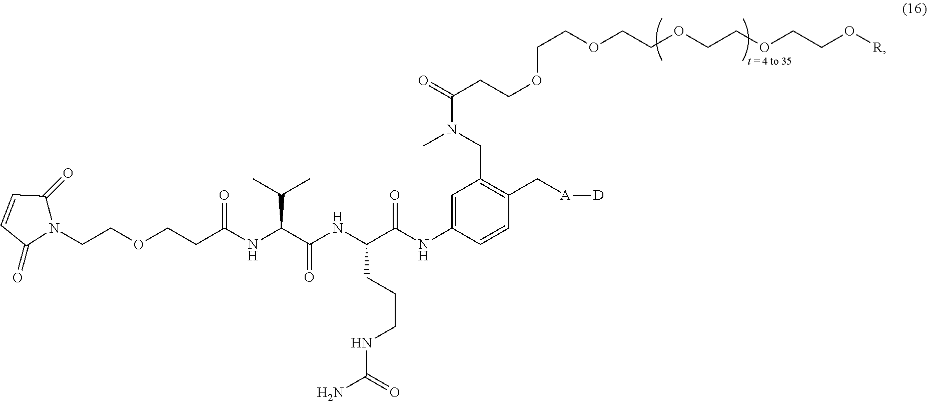
wherein:
each R independently is H, —CH₃ or —CH₂CH₂C(═O)OH;
A is a bond, —OC(═O)—*, 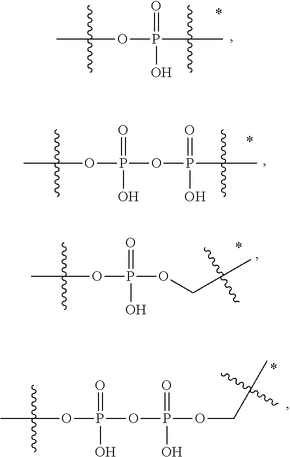 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor, or 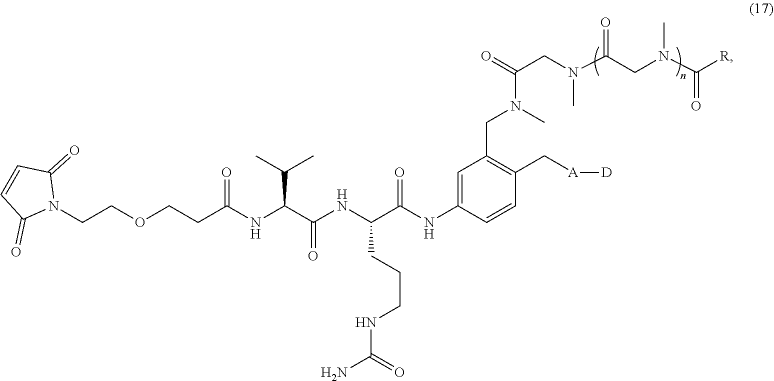
wherein:
each R independently is H, —CH₃ or —CH₂CH₂C(═O)OH;
A is a bond, —OC(═O)—*, 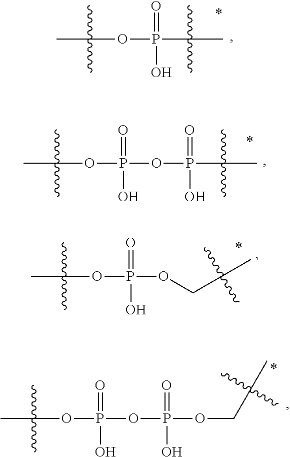 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D;
n is an integer between 2 and 24; and
D is a Bcl-xL inhibitor, or 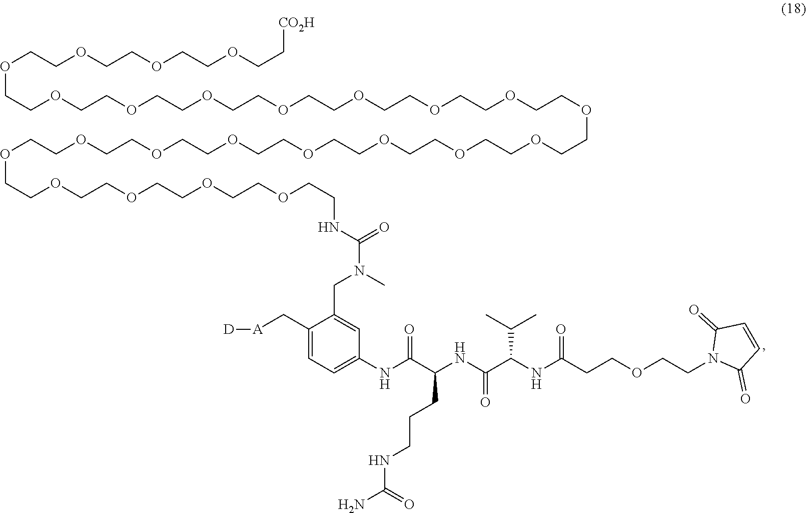
wherein:
A is a bond, —OC(═O)—*, 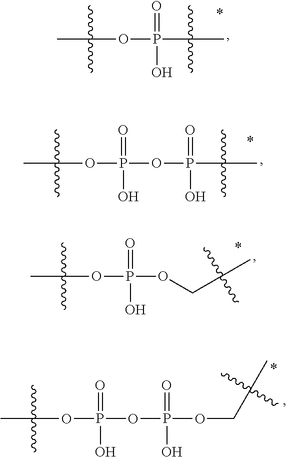 [SMILES: *OP(*)(=O)O ; *OP(=O)(O)OP(*)(=O)O ; *COP(=O)(O)O* ; *COP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a Bcl-xL inhibitor; or
(19)-(L-D) is formed from a compound selected from:

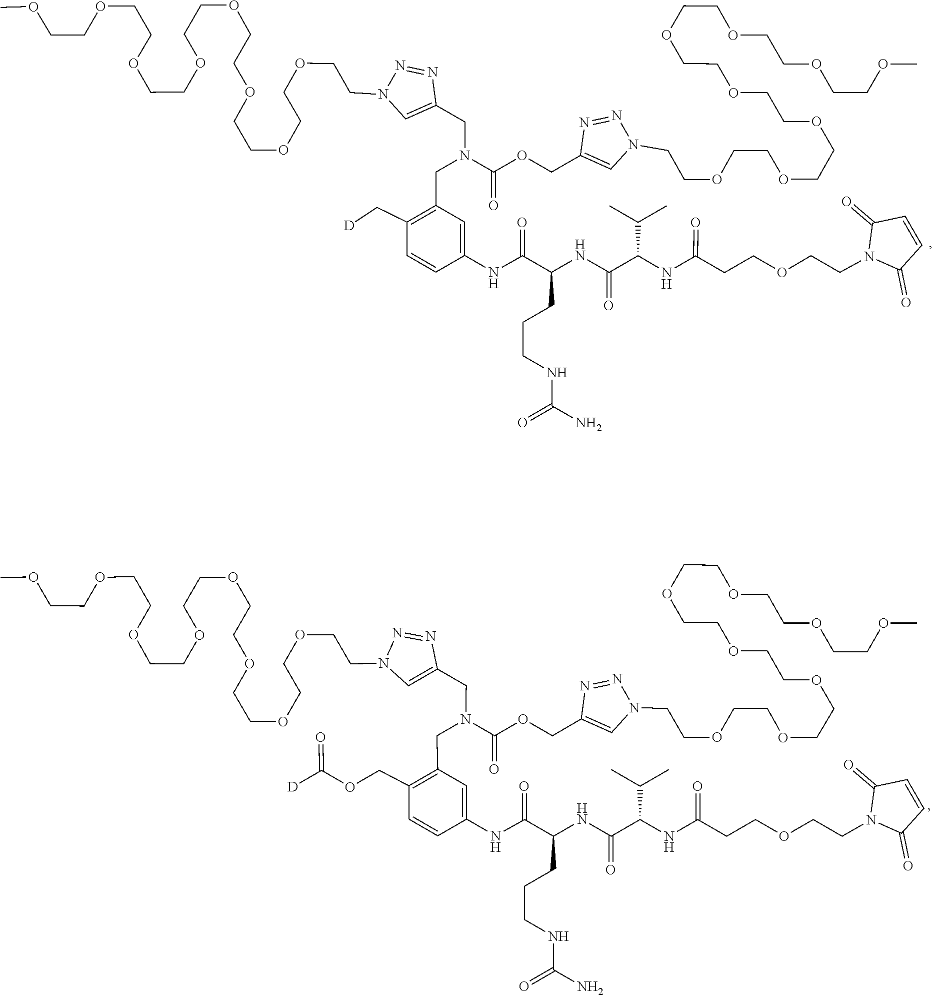 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOC)nn1)C(=O)OCc1cn(CCOCCOCCOCCOCCOCCOCCOCCOC)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOC)nn1)C(=O)OCc1cn(CCOCCOCCOCCOCCOCCOCCOCCOC)nn1]

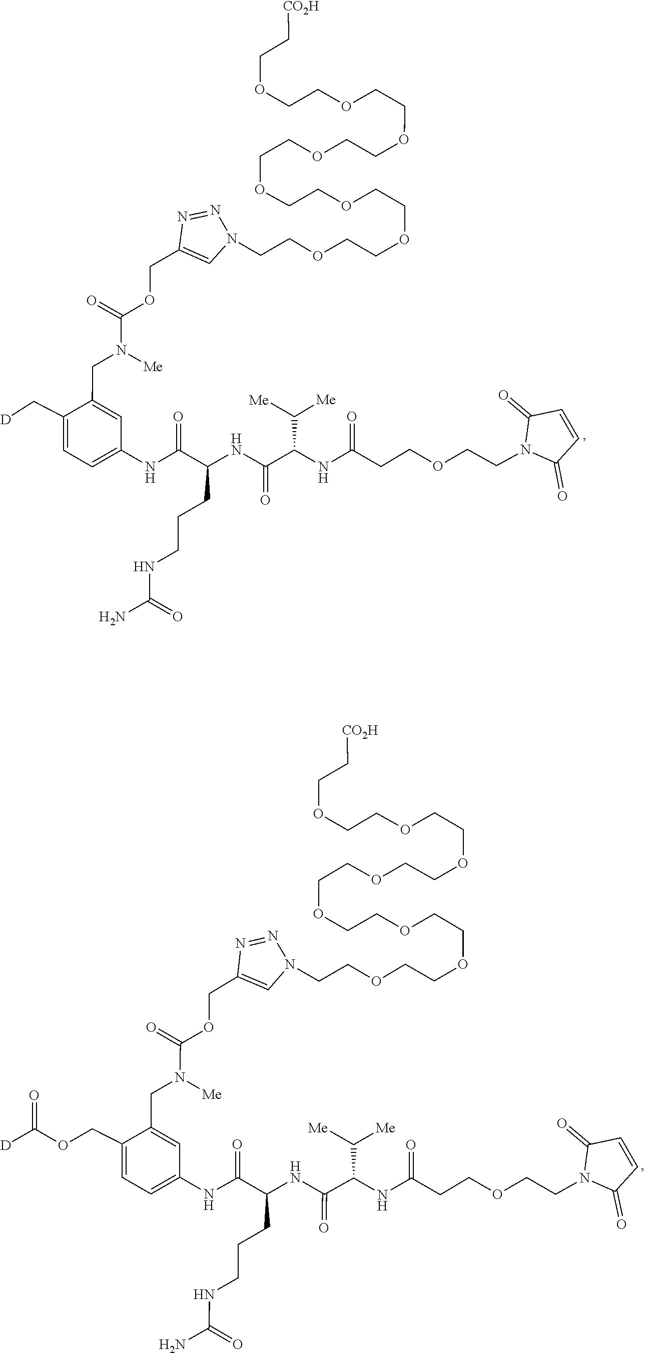 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCC(=O)O)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCC(=O)O)nn1]

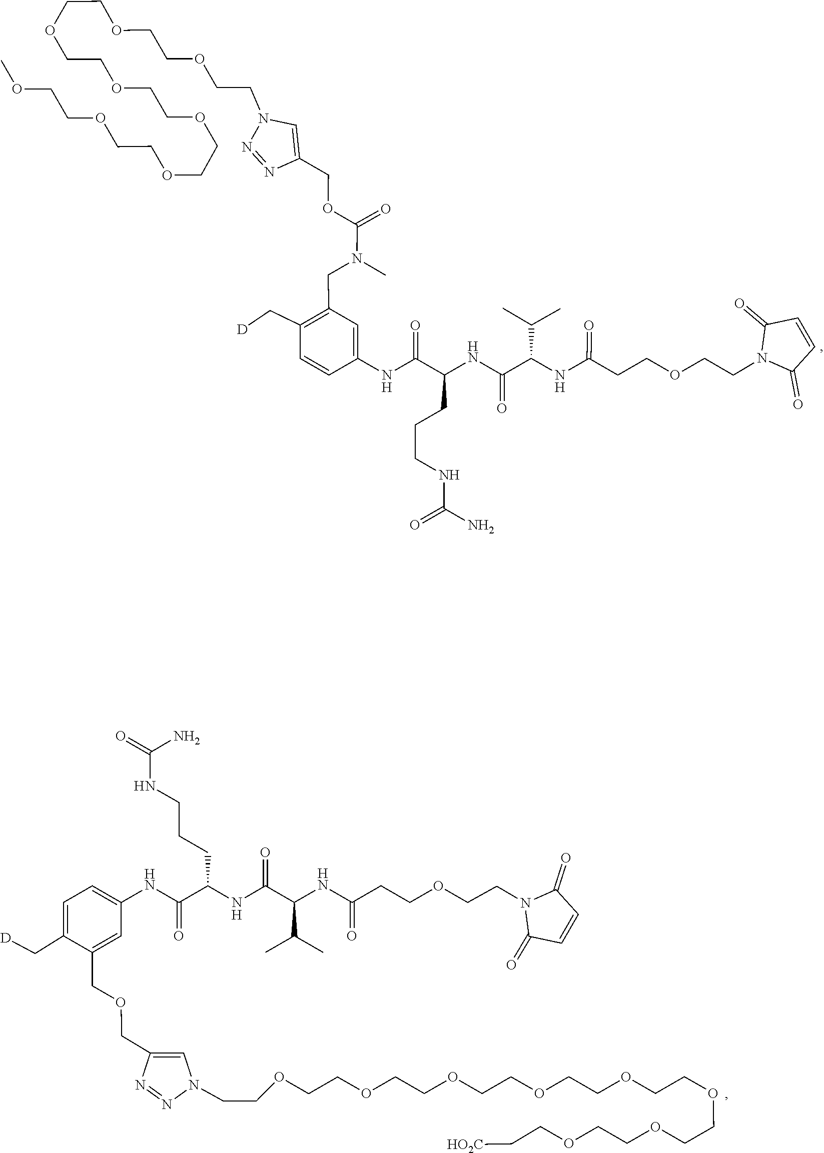 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOC)nn1 ; [2H]Cc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOCCC(=O)O)nn1]

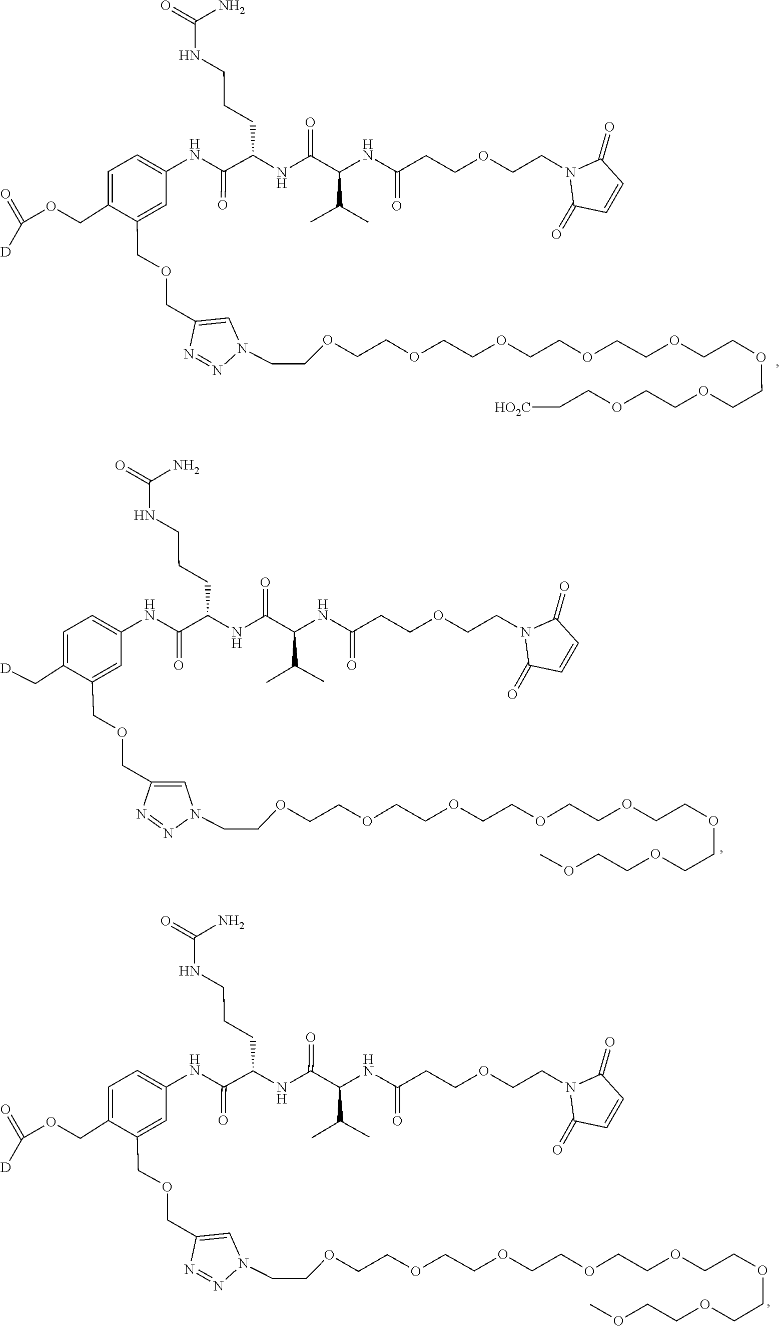 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOCCC(=O)O)nn1 ; [2H]Cc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOC)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOC)nn1]

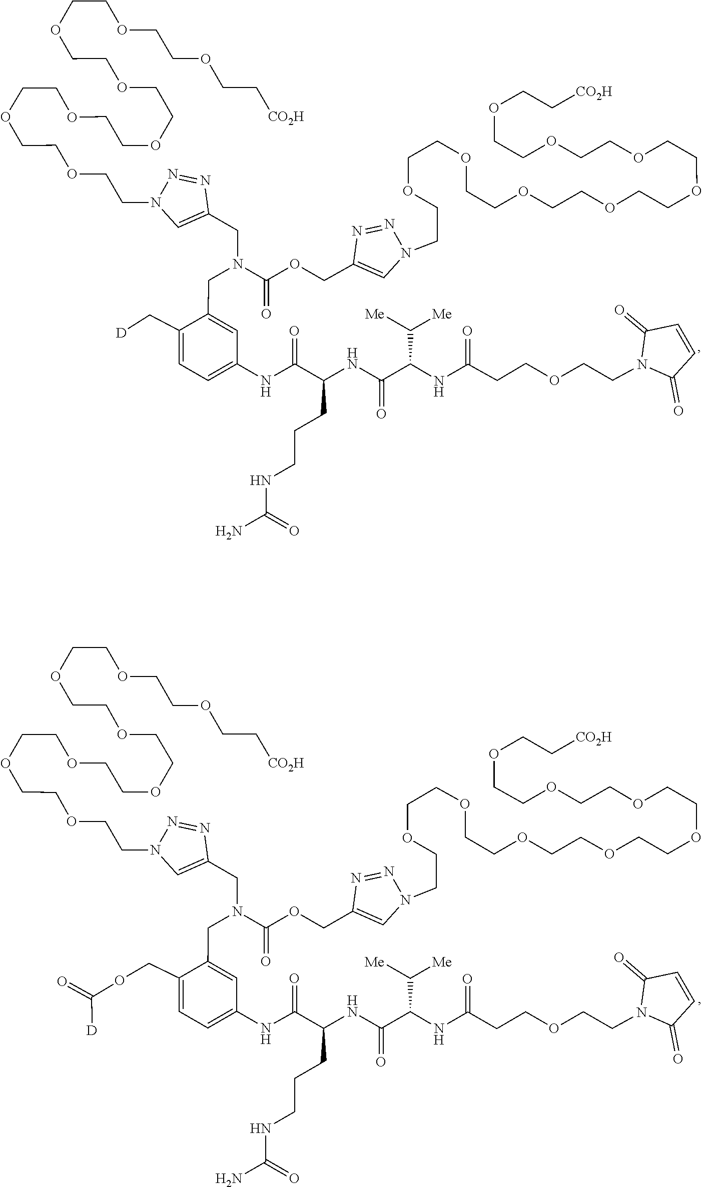 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCC(=O)O)nn1)C(=O)OCc1cn(CCOCCOCCOCCOCCOCCOCCOCCOCCC(=O)O)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCC(=O)O)nn1)C(=O)OCc1cn(CCOCCOCCOCCOCCOCCOCCOCCOCCC(=O)O)nn1]

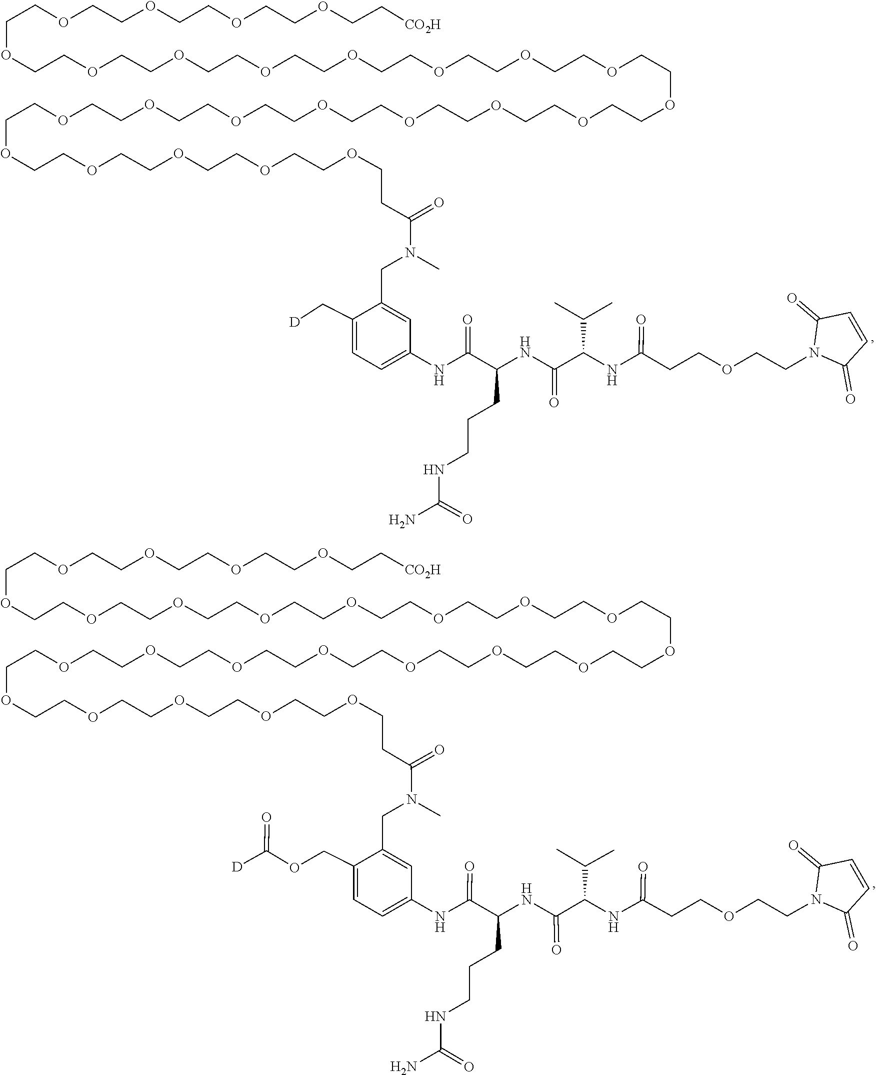

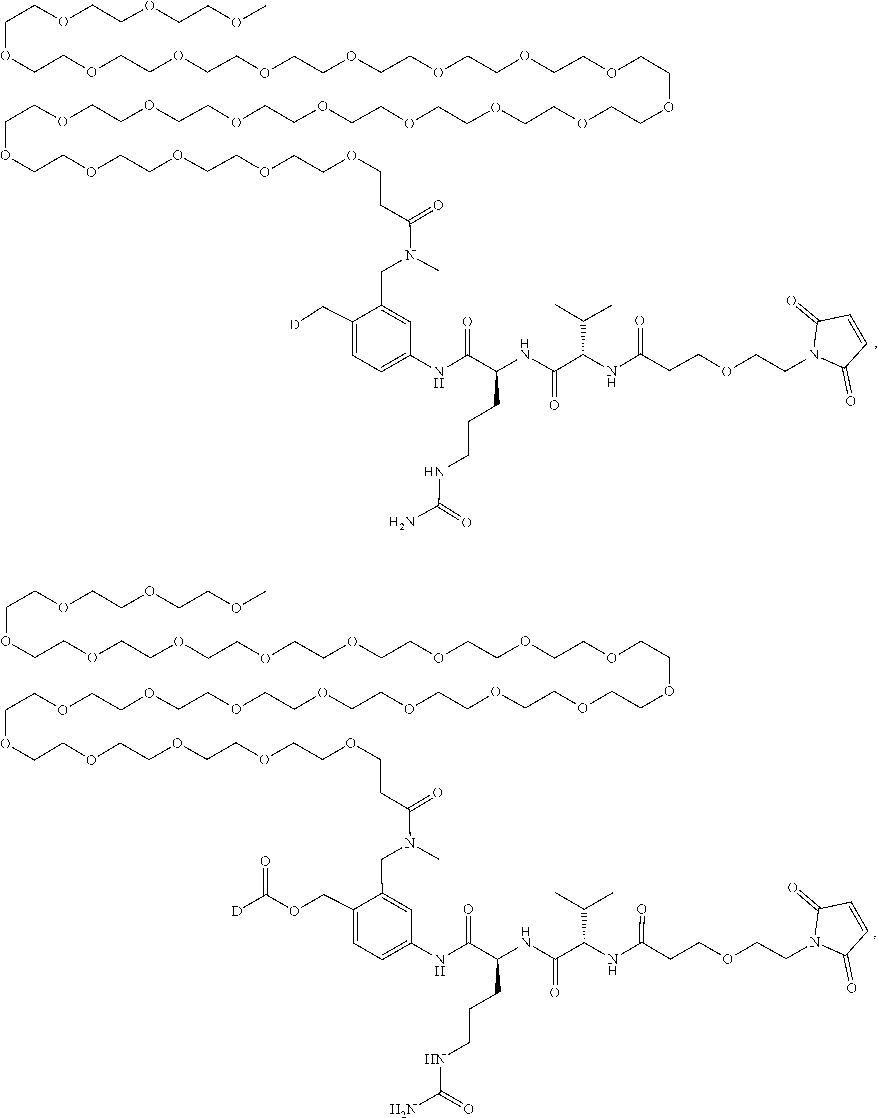 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOC ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOC]

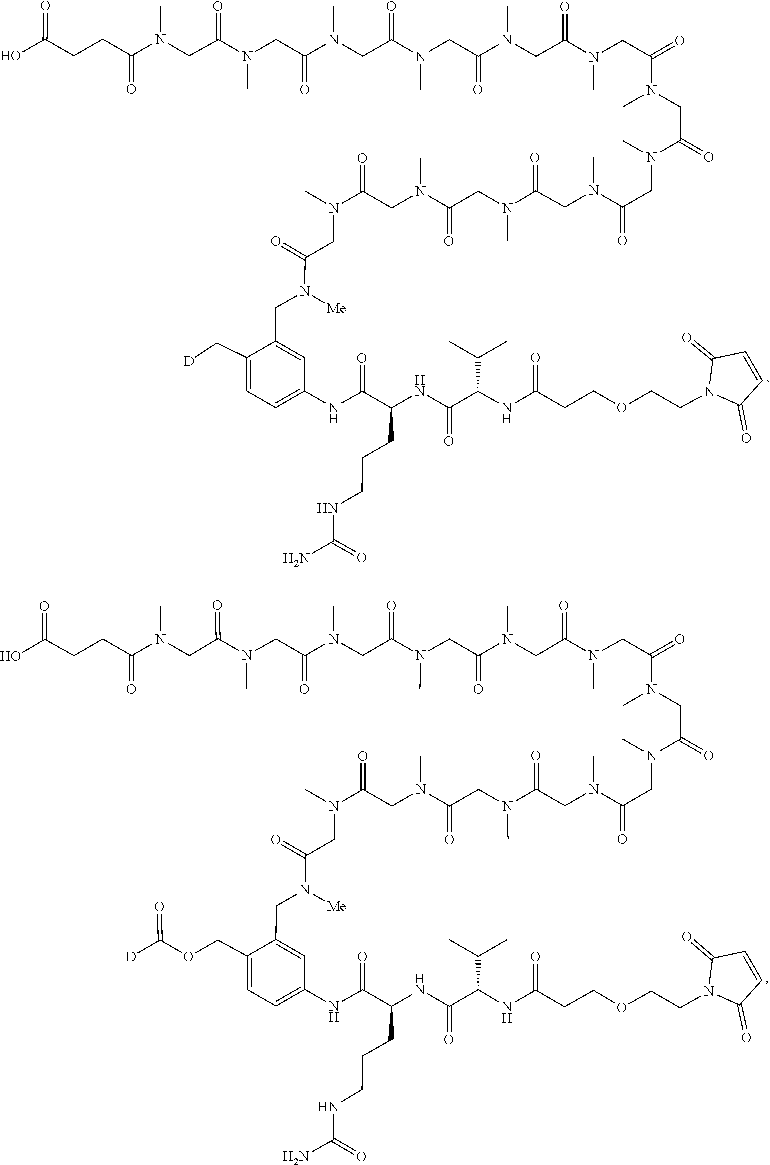 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O]

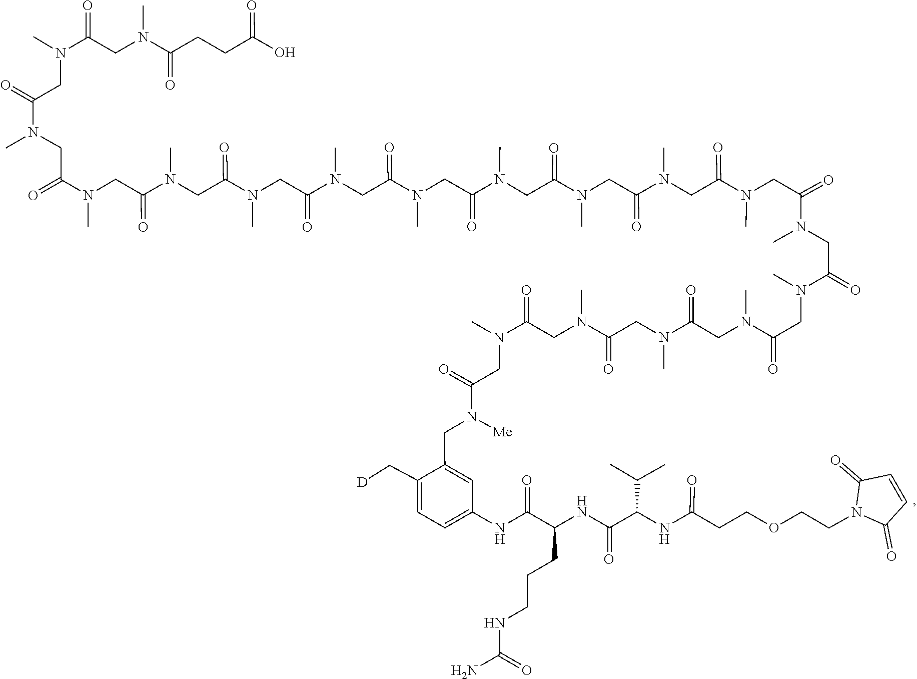 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O]

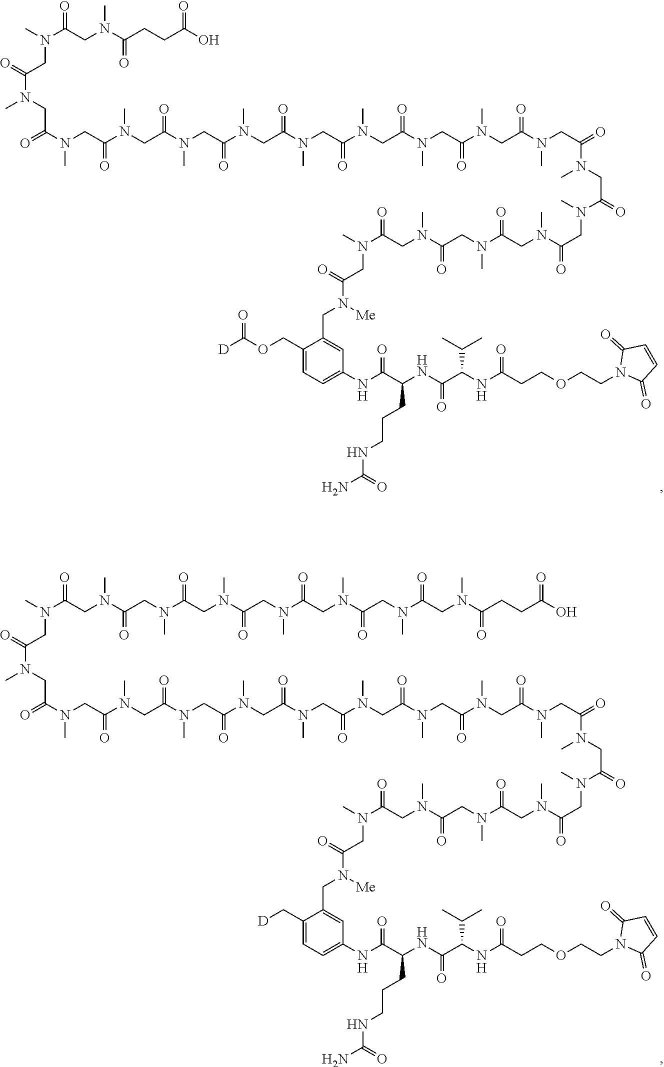

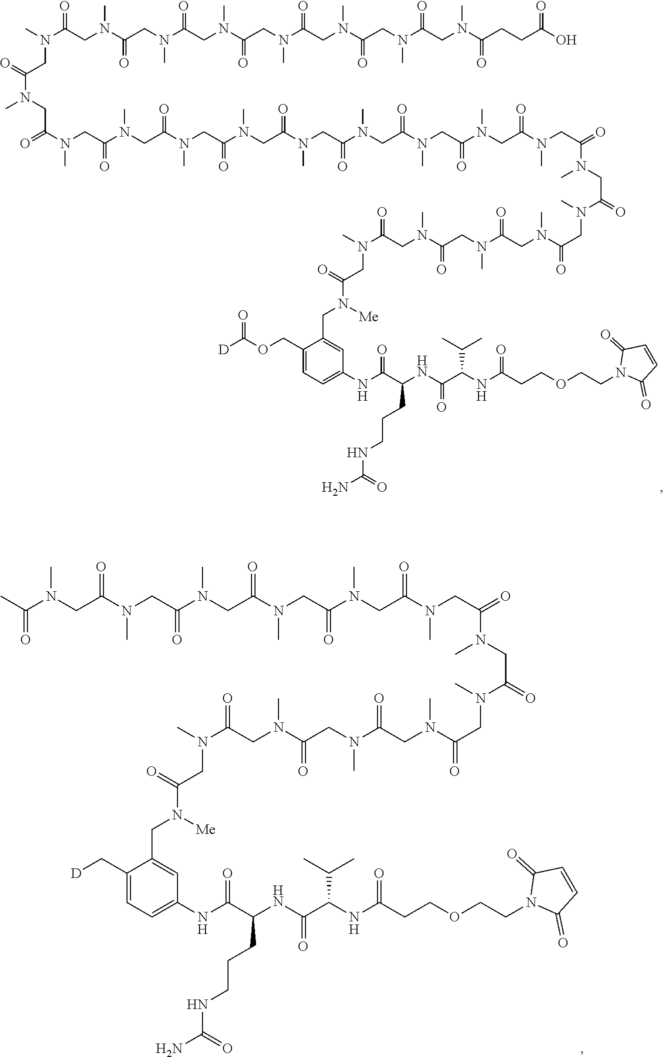

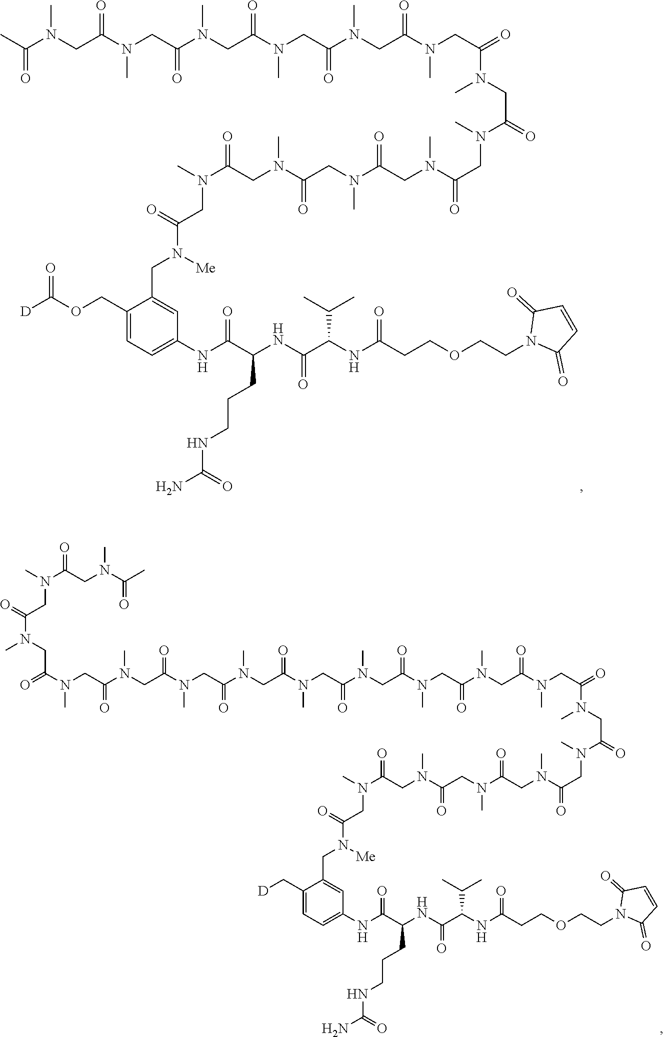 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O]

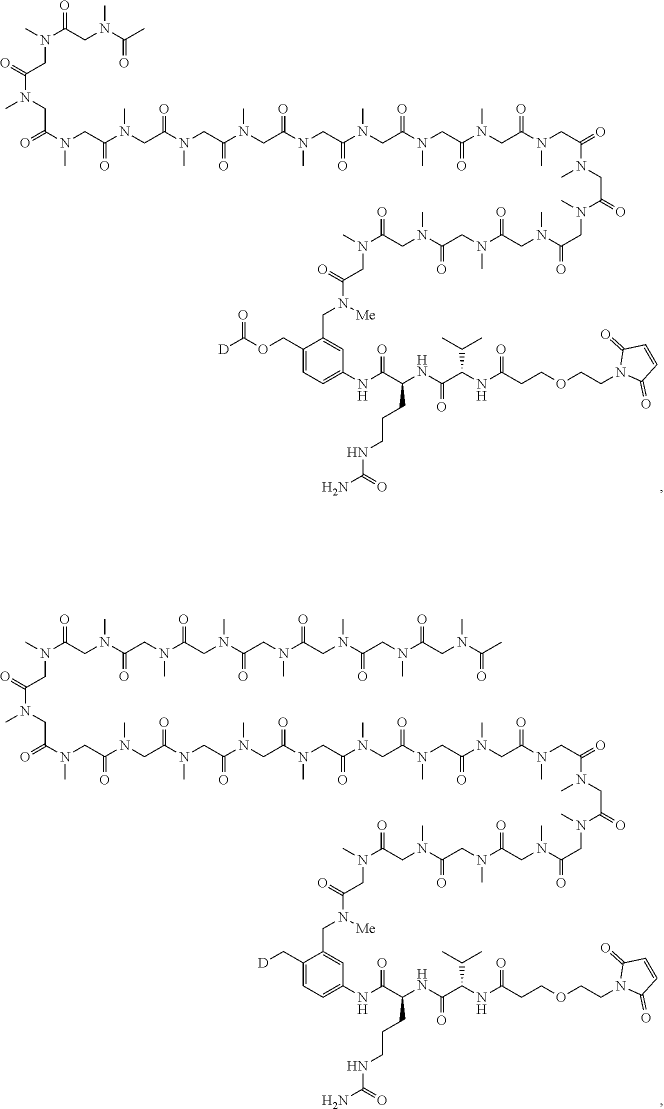

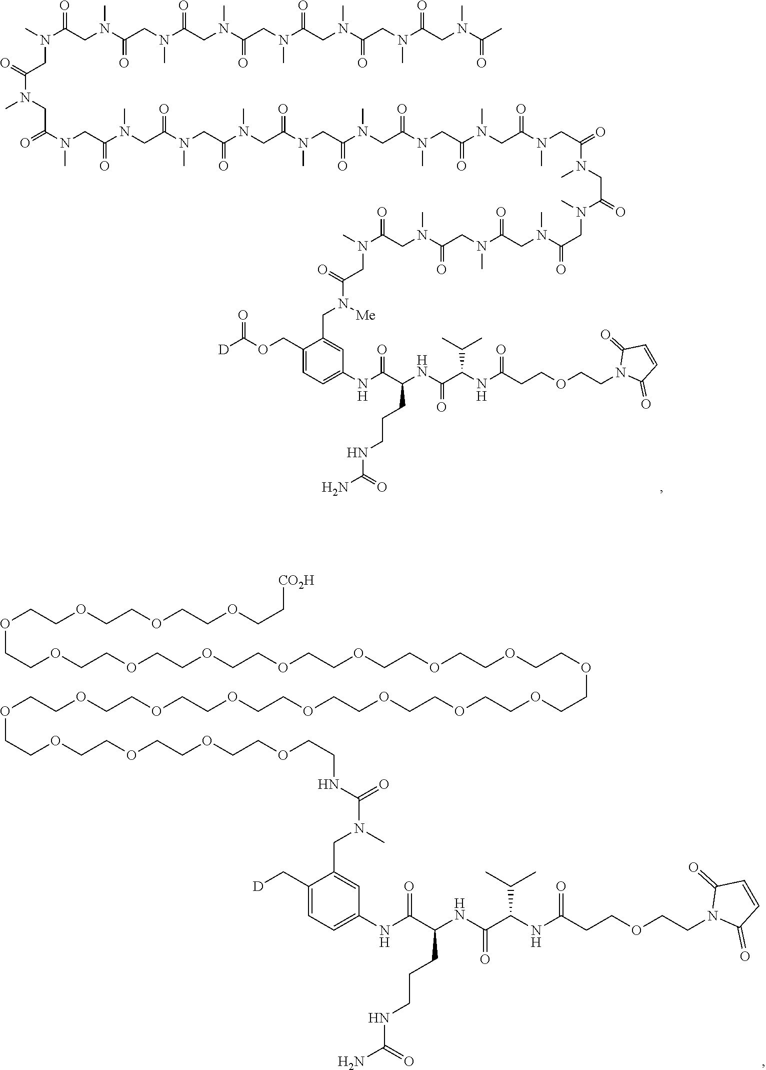

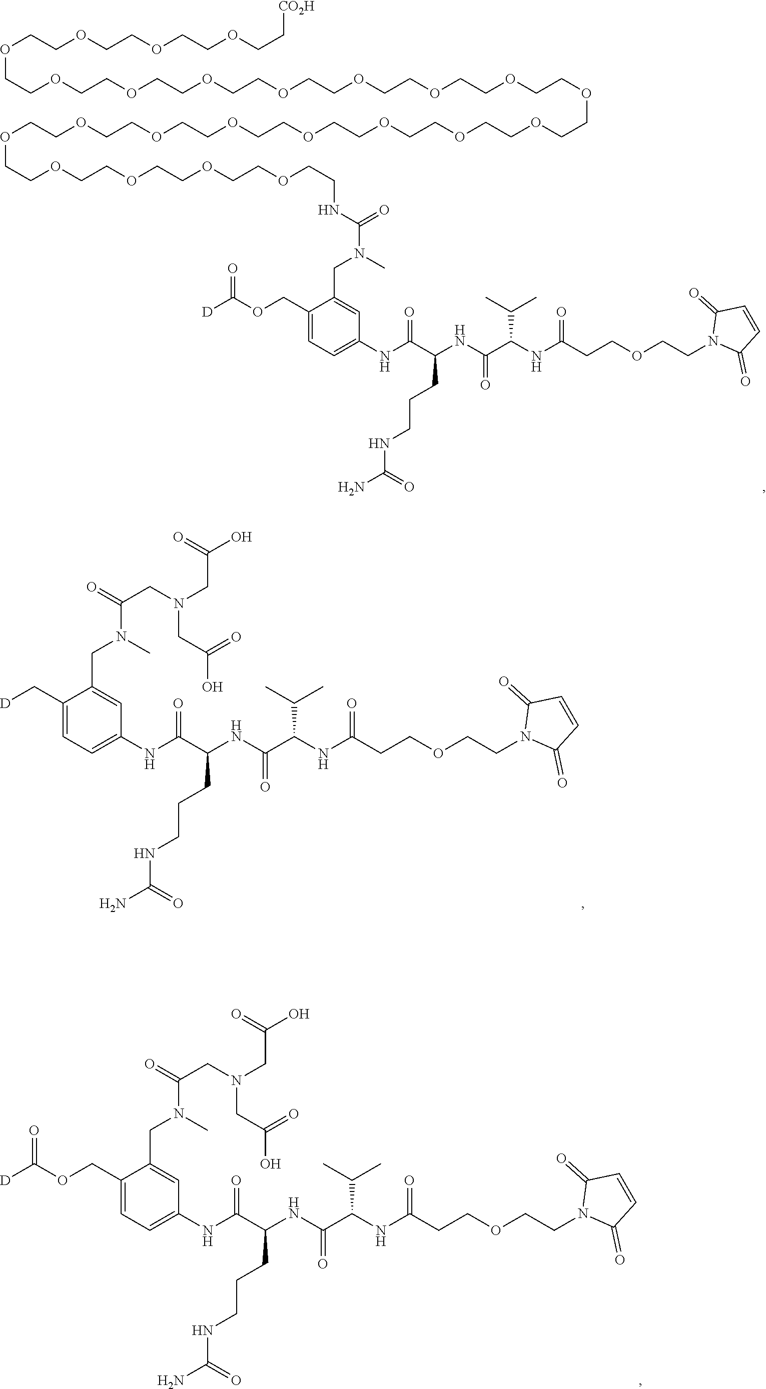 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O ; [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(CC(=O)O)CC(=O)O ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(CC(=O)O)CC(=O)O]

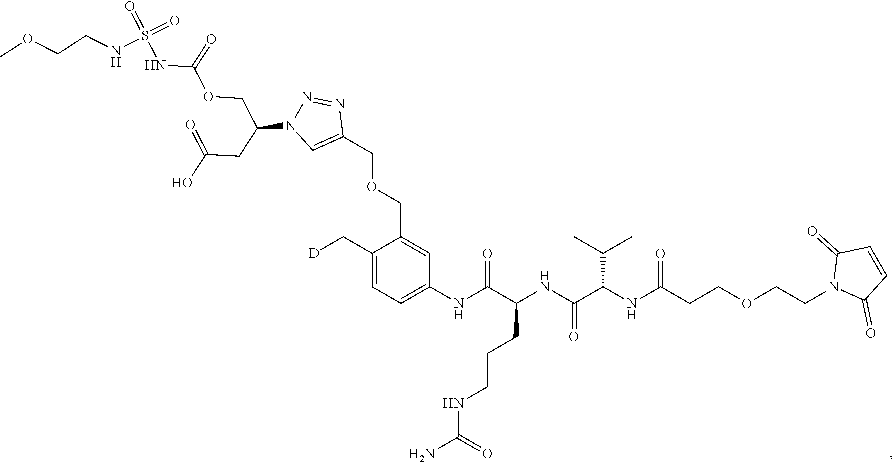 [SMILES: [2H]Cc1ccc(NC(=O)[C@H](CCCNC(N)=O)NC(=O)[C@@H](NC(=O)CCOCCN2C(=O)C=CC2=O)C(C)C)cc1COCc1cn([C@H](COC(=O)NS(=O)(=O)NCCOC)CC(=O)O)nn1]

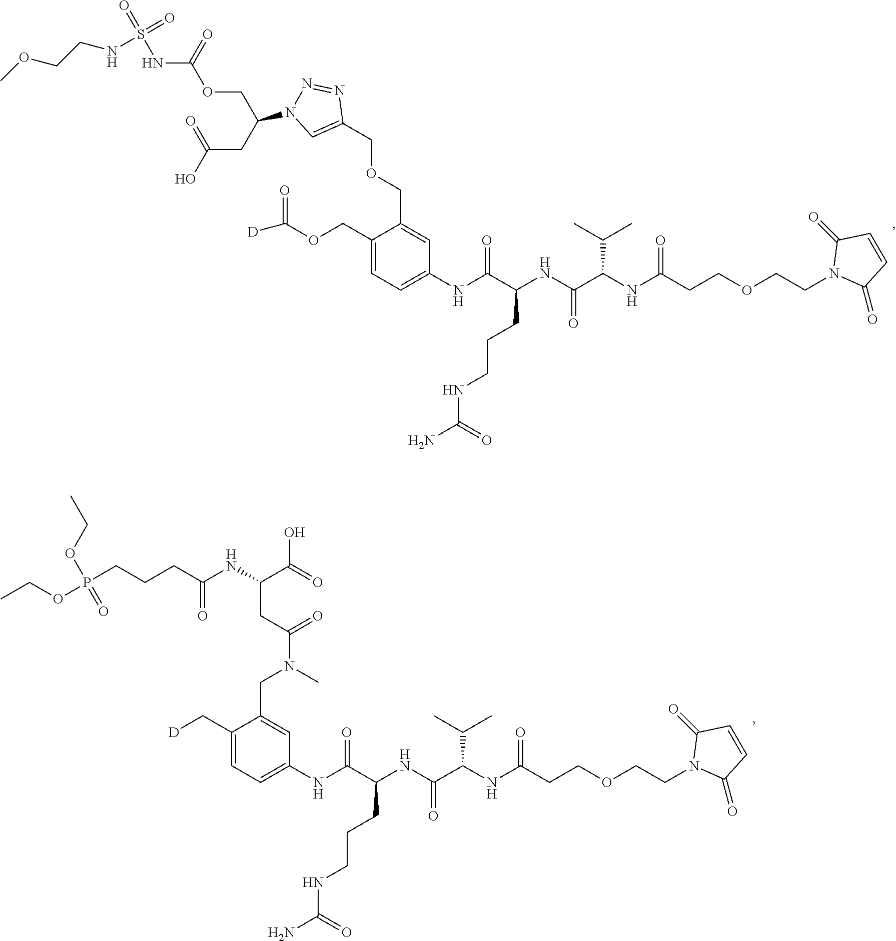 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1COCc1cn([C@H](COC(=O)NS(=O)(=O)NCCOC)CC(=O)O)nn1 ; [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)CCCP(=O)(OCC)OCC)C(=O)O]

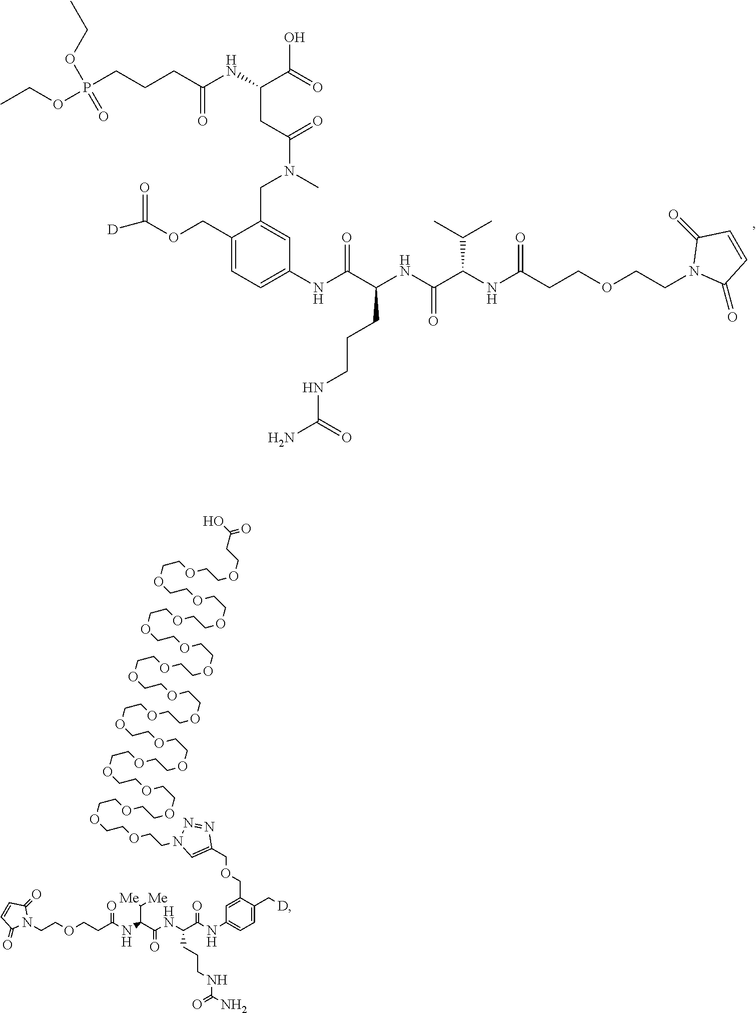

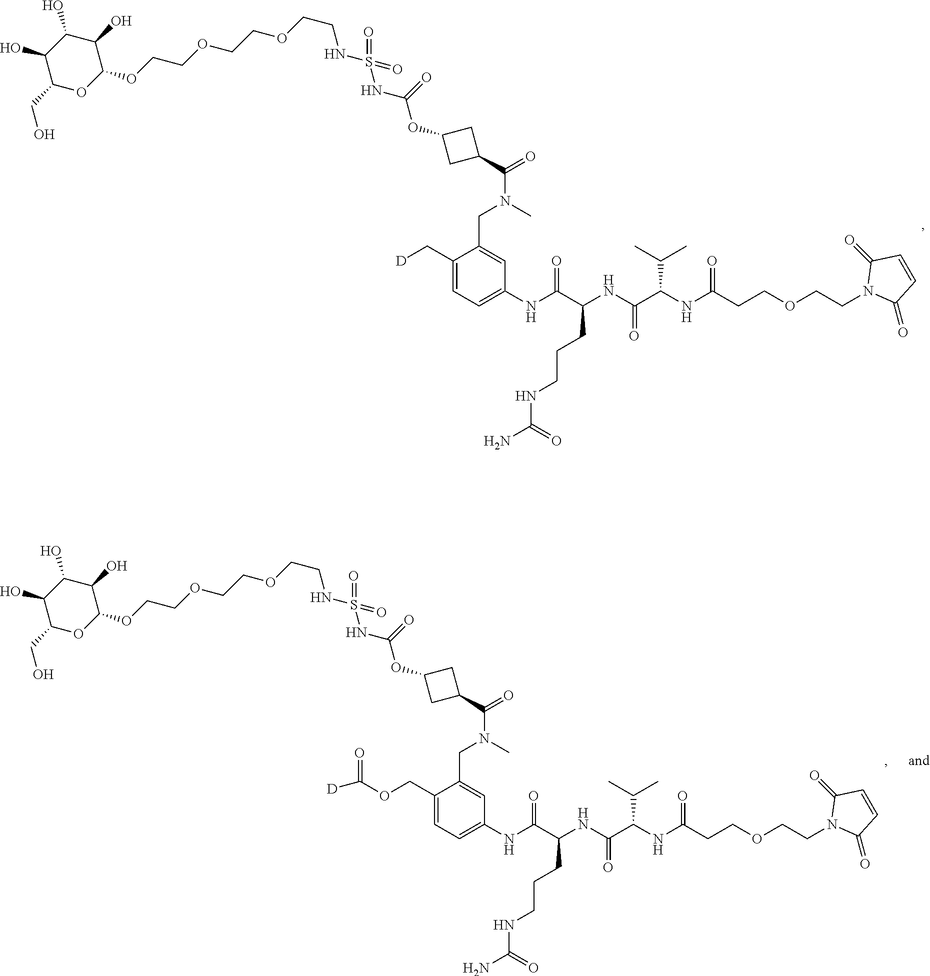 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@H]1C[C@H](OC(=O)NS(=O)(=O)NCCOCCOCCO[C@@H]2O[C@H](CO)[C@@H](O)[C@H](O)[C@H]2O)C1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@H]1C[C@H](OC(=O)NS(=O)(=O)NCCOCCOCCO[C@@H]2O[C@H](CO)[C@@H](O)[C@H](O)[C@H]2O)C1]

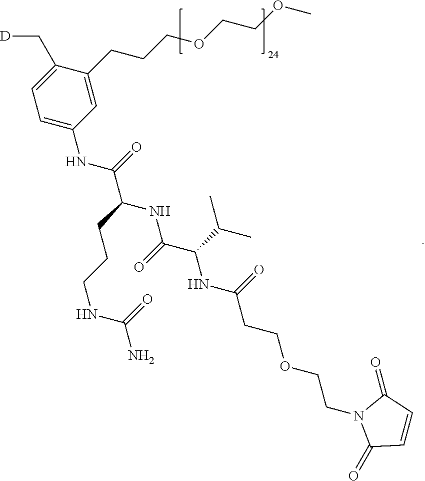

38. The antibody-drug conjugate of claim 37, wherein:
(1) A is a bond and/or R is —CH₃ or —CH₂CH₂COOH; or
(2) A is —OC(═O)—* and/or R is —CH₃ or —CH₂CH₂COOH.

39-40. (canceled)

41. The antibody-drug conjugate of claim 38, wherein D comprises a compound of Formula (I): 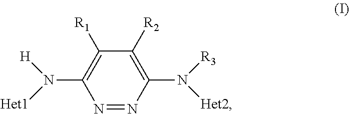
or
or an enantiomer, a diastereoisomer, and/or a pharmaceutically acceptable salt of any one of the foregoing, wherein:
R₁ and R₂ independently of one another represent a group selected from: hydrogen; linear or branched C₁-C₆alkyl optionally substituted by a hydroxyl or a C₁-C₆alkoxy group; C₃-C₆cycloalkyl; trifluoromethyl; linear or branched C₁-C₆alkylene-heterocycloalkyl wherein the heterocycloalkyl group is optionally substituted by a linear or branched C₁-C₆alkyl group;
or R₁ and R₂ form with the carbon atoms carrying them a C₃-C₆cycloalkylene group,
R₃ represents a group selected from: hydrogen; C₃-C₆cycloalkyl; linear or branched C₁-C₆alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and: 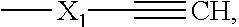
Rₐ and R_{b} independently of one another represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl groups; C₁-C₆alkylene-SO₂OH; C₁-C₆alkylene-SO₂O⁻; C₁-C₆alkylene-COOH; C₁-C₆alkylene-PO(OH)₂; C₁-C₆alkylene-NR_{d}Rₑ; C₁-C₆alkylene-N⁺R_{d}RₑR_{f}; C₁-C₆alkylene-phenyl wherein the phenyl may be substituted by a C₁-C₆alkoxy group;
the group: 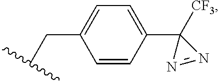 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₈heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈heterocycloalkyl,
Het₁ represents a group selected from: 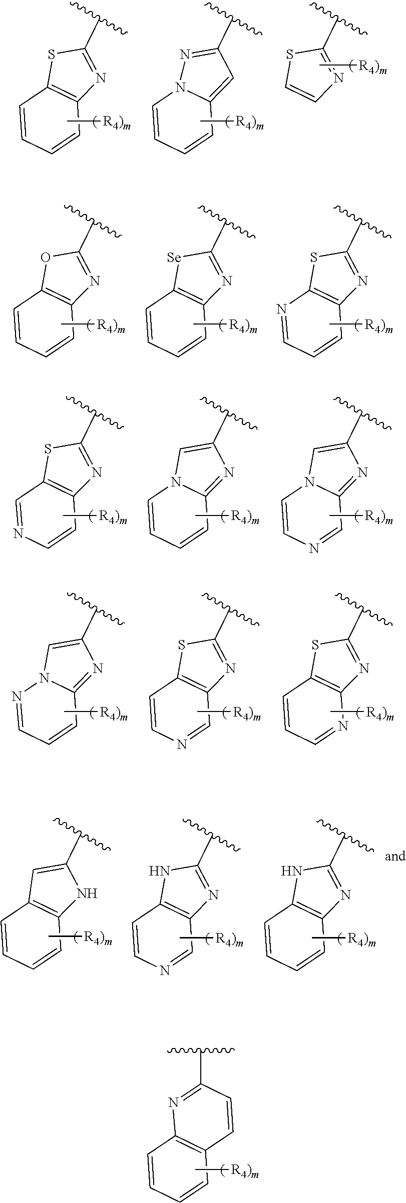
Het₂ represents a group selected from: 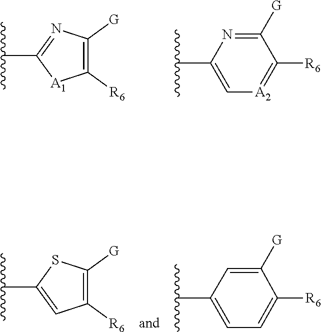
A₁ is —NH—, —N(C₁-C₃alkyl), O, S or Se,
A₂ is N, CH or C(R₅),
G is selected from the group consisting of:
—C(O)ORG, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, C₁-C₆alkyl optionally substituted by a hydroxyl group, halogen, —NO₂, and —CN, in which:
R_{G1} and R_{G2} at each occurrence are each independently selected from the group consisting of hydrogen, C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl;
R_{G3} is selected from the group consisting of C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl; or
R_{G1} and R_{G2}, together with the atom to which each is attached are combined to form a C₃-C₈heterocycloalkyl; or in the alternative, G is selected from the group consisting of: 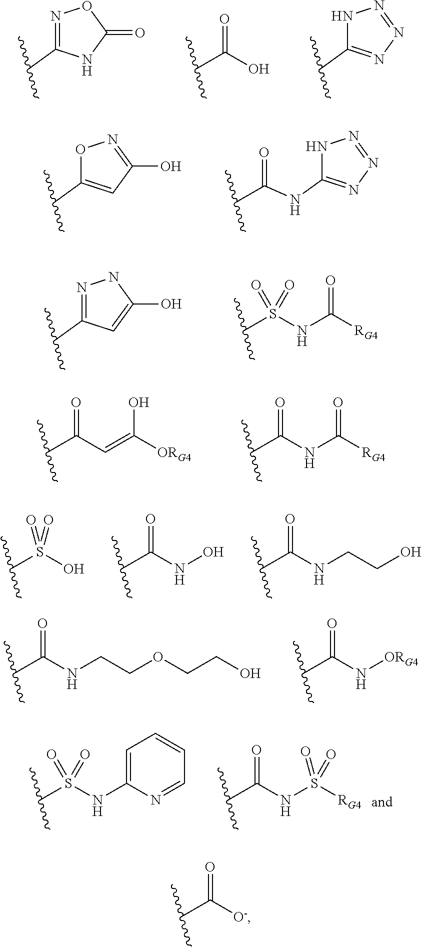
wherein R_{G4} is selected from hydrogen, C₁-C₆alkyl optionally substituted by 1 to 3 halogen atoms, C₂-C₆alkenyl, C₂-C₆alkynyl and C₃-C₆cycloalkyl,
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; C₂-C₆alkenyl; C₂-C₆alkynyl; halogen or —CN,
R₆ represents a group selected from:
hydrogen;
C₂-C₆alkenyl;
—X₂—O—R₇; 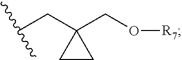
—X₂—NSO₂—R₇;
—C═C(R₉)—Y₁—O—R₇;
C₃-C₆cycloalkyl;
C₃-C₆heterocycloalkyl optionally substituted by a hydroxyl group;
C₃-C₆cycloalkylene-Y₂—R₇;
C₃-C₆heterocycloalkylene-Y₂—R₇ group,
an heteroarylene-R₇ group optionally substituted by a linear or branched C₁-C₆alkyl group,
R₇ represents a group selected from: linear or branched C₁-C₆alkyl group;
(C₃-C₆) cycloalkylene-R₈; or: 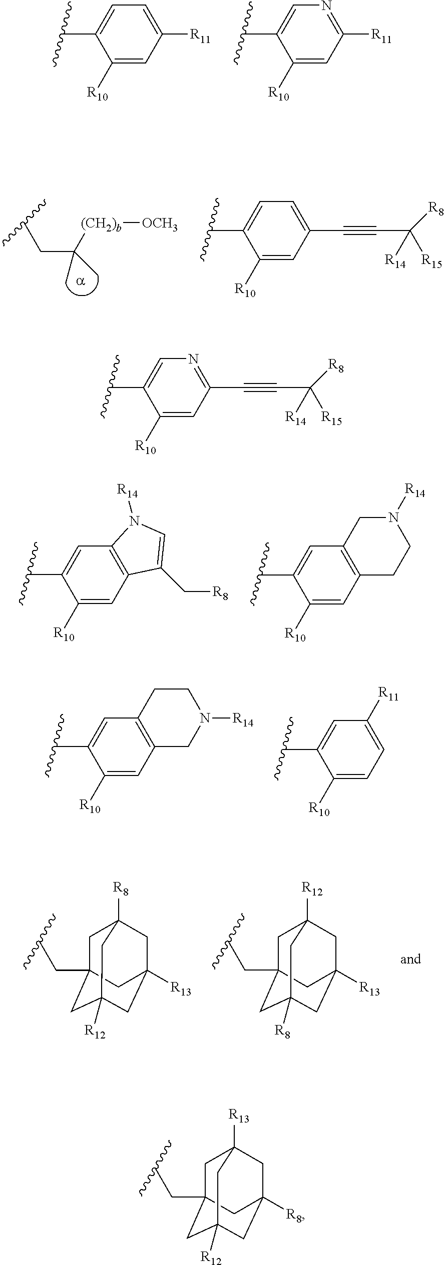
wherein Cy represents a C₃-C₈cycloalkyl,
R₈ represents a group selected from: hydrogen; linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and: 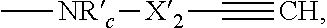
R₉ represents a group selected from linear or branched C₁-C₆alkyl, trifluoromethyl, hydroxyl, halogen, C₁-C₆alkoxy,
R₁₀ represents a group selected from hydrogen, fluorine, chlorine, bromine, —CF₃ and methyl,
R₁₁ represents a group selected from hydrogen, C₁-C₃alkylene-R₈, —O—C₁-C₃alkylene-R₈, —CO—NRₕRᵢ and —CH═CH—C₁-C₄alkylene-NRₕRᵢ, —CH═CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂ independently of one another, represent a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O⁻; C₁-C₆alkylene-COOH; C₁-C₆alkylene-PO(OH)₂; C₁-C₆alkylene-NR′_{d}R′ₑ; C₁-C₆alkylene-N⁺R′_{d}R′ₑR′_{f}; C₁-C₆alkylene-O—C₁-C₆alkylene-OH; C₁-C₆alkylene-phenyl wherein the phenyl may be substituted by a hydroxyl or a C₁-C₆alkoxy group;
the group: 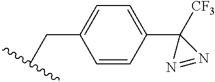 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, —NH—SO₂—,
m=0, 1 or 2,
p=1, 2, 3 or 4,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl, wherein one of the R₃ and R₈ groups, if present, is covalently attached to the linker, and wherein the valency of an atom is not exceeded by virtue of one or more substituents bonded thereto; optionally wherein
(i) R₁ is linear or branched C₁₋₆alkyl and R₂ is H;
(ii) G is selected from the group consisting of: —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN, in which:—R_{G1} and R_{G2} at each occurrence are each independently selected from the group consisting of hydrogen, C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl; —R_{G3} is selected from the group consisting of C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl; or R_{G1} and R_{G2}, together with the atom to which each is attached are combined to form a C₃-C₈heterocycloalkyl; or in the alternative, G is selected from the group consisting of: 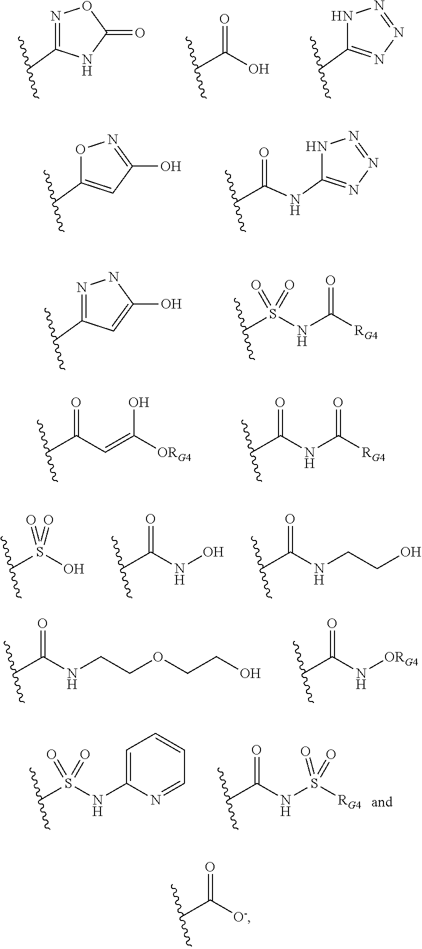
wherein R_{G4} is selected from C₁-C₆alkyl optionally substituted by 1 to 3 halogen atoms, C₂-C₆alkenyl, C₂-C₆alkynyl and C₃-C₆cycloalkyl.

42. (canceled)

43. The antibody-drug conjugate of claim 38, wherein D comprises a compound of Formula (II): 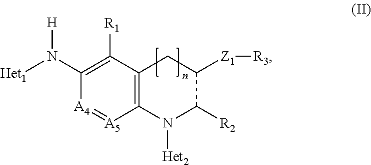
or an enantiomer, a diastereoisomer, and/or a pharmaceutically acceptable salt of any one of the foregoing, wherein:
n=0, 1 or 2, [symbol]
represents a single or a double bond,
A₄ and A₅ independently of one another represent a carbon or a nitrogen atom,
Z₁ represents a bond, —N(R)—, or —O—, wherein R represents a hydrogen or a linear or branched C₁-C₆alkyl,
R₁ represents a group selected from: hydrogen; linear or branched C₁-C₆alkyl optionally substituted by a hydroxyl or a C₁-C₆alkoxy group; C₃-C₆cycloalkyl; trifluoromethyl; linear or branched C₁-C₆alkylene-heterocycloalkyl wherein the heterocycloalkyl group is optionally substituted by a linear or branched C₁-C₆alkyl group;
R₂ represents a hydrogen or a methyl;
R₃ represents a group selected from: hydrogen; linear or branched C₁-C₄alkyl; —X₁—NRₐR_{b}; —X₁—N⁺RₐR_{b}R_{c}; —X₁—O—R_{c}; —X₁—COOR_{c}; —X₁—PO(OH)₂; —X₁—SO₂(OH); —X₁—N₃ and: 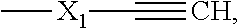
Rₐ and R_{b} independently of one another represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl groups; C₁-C₆alkylene-SO₂OH; C₁-C₆alkylene-SO₂O⁻; C₁-C₆alkylene-COOH; C₁-C₆alkylene-PO(OH)₂; C₁-C₆alkylene-NR_{d}Rₑ; C₁-C₆alkylene-N⁺R_{d}RₑR_{f}; C₁-C₆alkylene-phenyl wherein the phenyl may be substituted by a C₁-C₆alkoxy group;
the group: 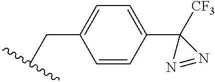 [SMILES: *Cc1ccc(C2(C(F)(F)F)N=N2)cc1]
or Rₐ and R_{b} form with the nitrogen atom carrying them a cycle B₁;
or Rₐ, R_{b} and R_{c} form with the nitrogen atom carrying them a bridged C₃-C₈heterocycloalkyl,
R_{c}, R_{d}, Rₑ, R_{f}, independently of one another represents a hydrogen or a linear or branched C₁-C₆alkyl group,
or R_{d} and Rₑ form with the nitrogen atom carrying them a cycle B₂,
or R_{d}, Rₑ and R_{f} form with the nitrogen atom carrying them a bridged C₃-C₈heterocycloalkyl,
Het₁ represents a group selected from: 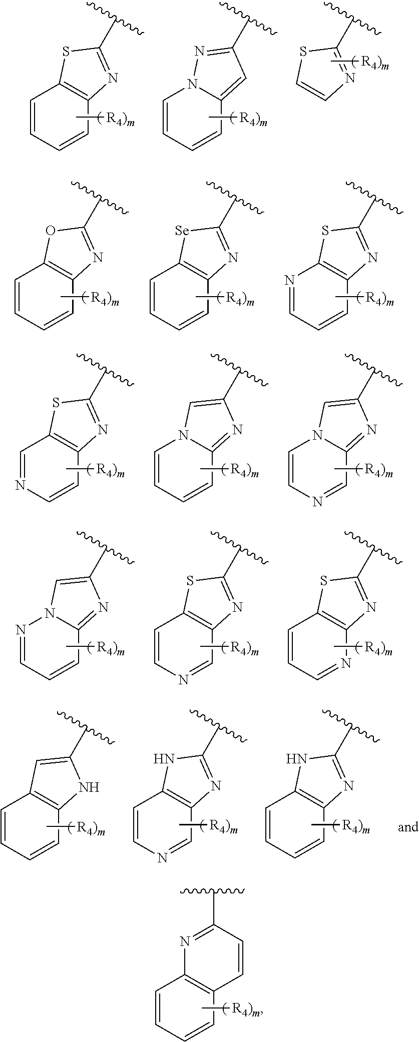
Het₂ represents a group selected from: 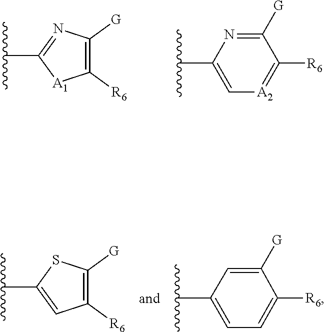
A₁ is —NH—, —N(C₁-C₃alkyl), O, S or Se,
A₂ is N, CH or C(R₅),
G is selected from the group consisting of:
—C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, C₁-C₆alkyl optionally substituted by a hydroxyl group, halogen, —NO₂, and —CN, in which:
R_{G1} and R_{G2} at each occurrence are each independently selected from the group consisting of hydrogen, C₁-C₆alkyl optionally substituted by 1 to 3 halogen atoms, C₂-C₆alkenyl, C₂-C₆alkynyl, C₃-C₆cycloalkyl, phenyl and —(CH₂)₁₋₄-phenyl;
R_{G3} is selected from the group consisting of C₁-C₆alkyl optionally substituted by 1 to 3 halogen atoms, C₂-C₆alkenyl, C₂-C₆alkynyl, C₃-C₆cycloalkyl, phenyl and —(CH₂)₁-4-phenyl; or
R_{G1} and R_{G2}, together with the atom to which each is attached are combined to form a C₃-C₈heterocycloalkyl; or in the alternative, G is selected from the group consisting of: 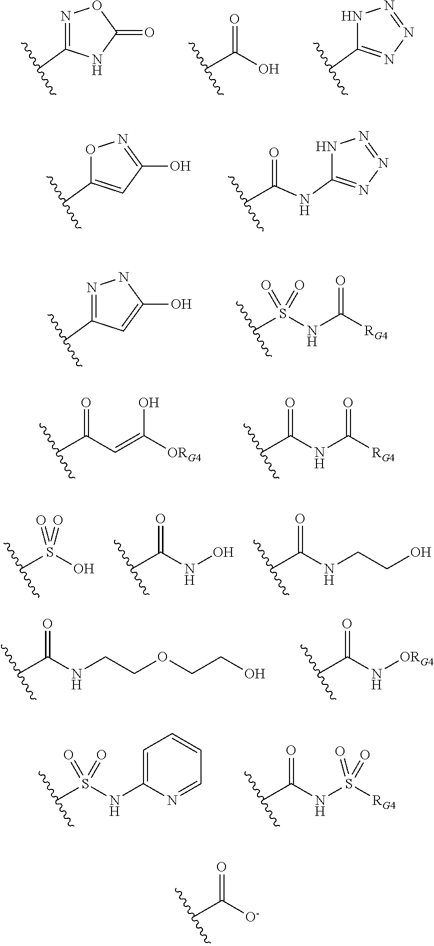
wherein R_{G4} is selected from hydrogen, C₁-C₆alkyl optionally substituted by 1 to 3 halogen atoms, C₂-C₆alkenyl, C₂-C₆alkynyl and C₃-C₆cycloalkyl,
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms; C₂-C₆alkenyl; C₂-C₆alkynyl; halogen or —CN,
R₆ represents a group selected from:
hydrogen;
—C₂-C₆alkenyl;
—X₂—O—R₇; 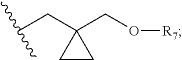
—X₂—NSO₂—R₇;
—C═C(R₉)—Y₁—O—R₇;
C₃-C₆cycloalkyl;
C₃-C₆heterocycloalkyl optionally substituted by a hydroxyl group;
C₃-C₆cycloalkylene-Y₂—R₇;
C₃-C₆heterocycloalkylene-Y₂—R₇ group,
an heteroarylene-R₇ group optionally substituted by a linear or branched C₁-C₆alkyl group,
R₇ represents a group selected from: linear or branched C₁-C₆alkyl group;
(C₃-C₆) cycloalkylene-R₈; or: 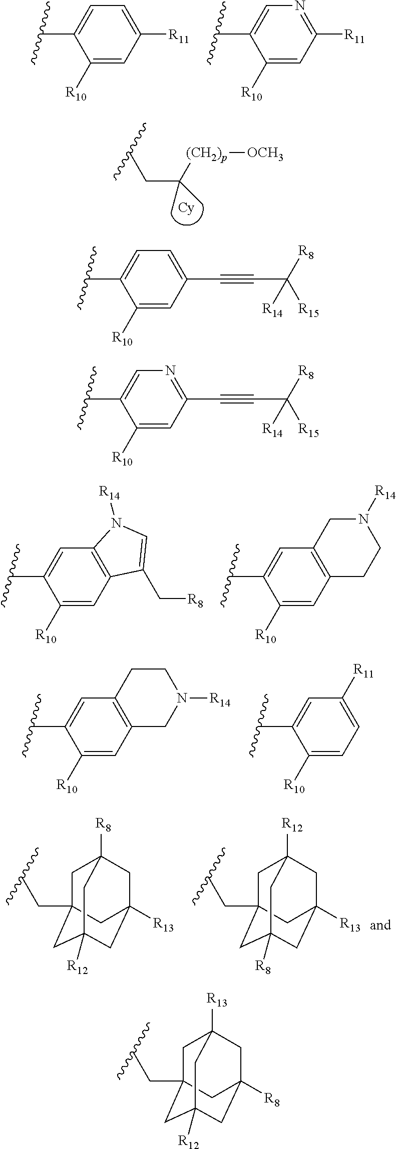
wherein Cy represents a C₃-C₈cycloalkyl,
R₈ represents a group selected from: hydrogen; linear or branched C₁-C₆alkyl, —NR′ₐR′_{b};
—NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}, —X′₂—NR′ₐR′_{b}, —NR′_{c}—X′₂—N₃ and: 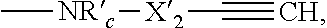
R₉ represents a group selected from linear or branched C₁-C₆alkyl, trifluoromethyl, hydroxyl, halogen, C₁-C₆alkoxy,
R₁₀ represents a group selected from hydrogen, fluorine, chlorine, bromine, —CF₃ and methyl,
R₁₁ represents a group selected from hydrogen, halogen, C₁-C₃alkylene-R₈, —O—C₁-C₃alkylene-R₈, —CO—NRₕRᵢ and —CH═CH—C₁-C₄alkylene-NRₕRᵢ, —CH—CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a
a cyclohexyl,
Rₕ and Rᵢ, independently of one another, represent a hydrogen or a linear or branched C₁-C₆alkyl group,
X₁ represents a linear or branched C₁-C₄alkylene group optionally substituted by one or two groups selected from trifluoromethyl, hydroxyl, halogen, C₁-C₆alkoxy,
X₂ represents a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O⁻; C₁-C₆alkylene-COOH; C₁-C₆alkylene-PO(OH)₂; C₁-C₆alkylene-NR′_{d}R′ₑ;
C₁-C₆alkylene-N⁺R′_{d}R′ₑR′_{f}; C₁-C₆alkylene-O—C₁-C₆alkylene-OH; C₁-C₆alkylene-phenyl wherein the phenyl may be substituted by a hydroxyl or a C₁-C₆alkoxy group;
the group: 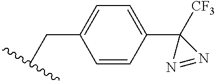 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈heterocycloalkyl,
Y₁ represents a linear or branched C₁-C₄alkylene,
Y₂ represents a bond, —O—, —O—CH₂—, —O—CO—, —O—SO₂—, —CH₂—, —CH₂—O, —CH₂—CO—, —CH₂—SO₂—, —C₂H₅—, —CO—, —CO—O—, —CO—CH₂—, —CO—NH—CH₂—, —SO₂—, —SO₂—CH₂—, —NH—CO—, —NH—SO₂—,
m=0, 1 or 2,
p=1, 2, 3 or 4,
B₁, B₂, B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl,
wherein one of the R₃ and R₄ groups, if present, is covalently attached to the linker, and
wherein the valency of an atom is not exceeded by virtue of one or more substituents bonded thereto; or
wherein A₁ and A₅ both represent a nitrogen atom, R₁ is linear or branched C₁₋₆alkyl; R₂ is H; n is 1; and [symbol] represents a single bond.

44-45. (canceled)

46. The antibody-drug conjugate of claim 38, wherein D comprises a compound of formula (IA) or (IIA): 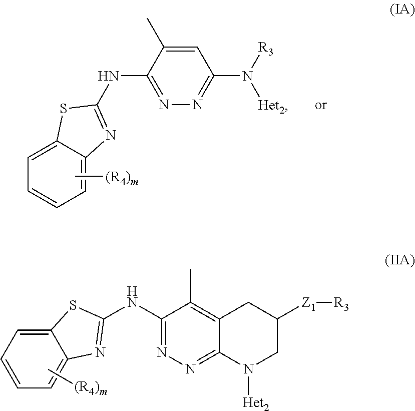
or an enantiomer, a diastereoisomer, and/or a pharmaceutically acceptable salt of any one of the foregoing, wherein:
Z₁ represents a bond or —O—,
R₃ represents a group selected from: hydrogen; C₃-C₆cycloalkyl; linear or branched C₁-C₆alkyl; —X₁—NRₐR_{b}; —X₁—N⁺RₐR_{b}R_{c}; and —X₁—O—R_{c},
Rₐ and R_{b} independently of one another represent a group selected from: hydrogen; linear or branched C₁-C₆alkyl optionally substituted by one or two hydroxyl groups; and C₁-C₆alkylene-SO₂O⁻,
R_{c} represents a hydrogen or a linear or branched C₁-C₆alkyl group,
Het₂ represents a group selected from: 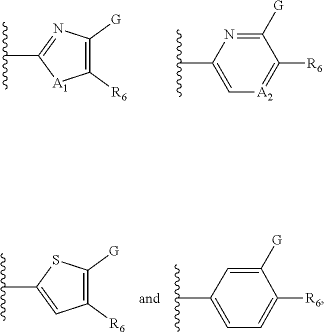
A₁ is —NH—, —N(C₁-C₃alkyl), O, S or Se,
A₂ is N, CH or C(R₅),
G is selected from the group consisting of:
—C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, C₁-C₆alkyl optionally substituted by a hydroxyl group, halogen, —NO₂, and —CN, in which:
R_{G1} and R_{G2} at each occurrence are each independently selected from the group consisting of hydrogen, and C₁-C₆alkyl optionally substituted by 1 to 3 halogen atoms;
R_{G3} is C₁-C₆alkyl optionally substituted by 1 to 3 halogen atoms; or
R_{G1} and R_{G2}, together with the atom to which each is attached are combined to form a C₃-C₈heterocycloalkyl;
R₄ represents a hydrogen, fluorine, chlorine or bromine atom, a methyl, a hydroxyl or a methoxy group,
R₅ represents a group selected from: C₁-C₆alkyl optionally substituted by 1 to 3 halogen atoms;
halogen or —CN,
R₆ represents a group selected from:
—X₂—O—R₇; and
an heteroarylene-R₇ group optionally substituted by a linear or branched C₁-C₆alkyl group, R₇ represents a group selected from: linear or branched C₁-C₆alkyl group;
(C₃-C₆) cycloalkylene-R₈; or: 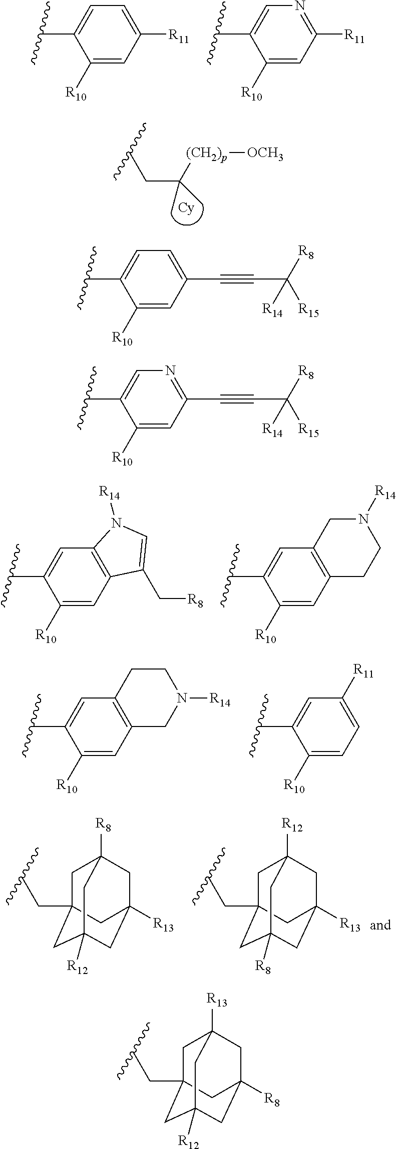
wherein Cy represents a C₃-C₈cycloalkyl,
R₈ represents a group selected from: hydrogen; linear or branched C₁-C₆alkyl, —NR′ₐR′_{b}; —NR′ₐ—CO—OR′_{c}; —NR′ₐ—CO—R′_{c}; —N⁺R′ₐR′_{b}R′_{c}; —O—R′_{c}; —NH—X′₂—N⁺R′ₐR′_{b}R′_{c}; —O—X′₂—NR′ₐR′_{b}; —X′₂—NR′ₐR′_{b}: —NR′_{c}—X′₂—N₃ and: 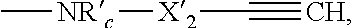
R₁₀ represents a group selected from hydrogen, fluorine, chlorine, bromine, —CF₃ and methyl,
R₁₁ represents a group selected from hydrogen, C₁-C₆alkylene-R₈, —O—C₁-C₆alkylene-R₈, —CO—NRₕRᵢ and —CH═CH—C₁-C₄alkylene-NRₕRᵢ, —CH—CH—CHO, C₃-C₈cycloalkylene-CH₂—R₈, C₃-C₈heterocycloalkylene-CH₂—R₈,
R₁₂ and R₁₃, independently of one another, represent a hydrogen atom or a methyl group,
R₁₄ and R₁₅, independently of one another, represent a hydrogen or a methyl group, or R₁₄ and R₁₅ form with the carbon atom carrying them a cyclohexyl,
Rₕ and Rᵢ, independently of one another, represent a hydrogen or a linear or branched C₁-C₆alkyl group,
X₁ and X₂ independently of one another, represent a linear or branched
C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; heterocycloalkyl; —SO₂-phenyl wherein the phenyl may be substituted by a linear or branched C₁-C₆alkyl; linear or branched C₁-C₆alkyl optionally substituted by one or two hydroxyl or C₁-C₆alkoxy groups; C₁-C₆alkylene-SO₂OH; C₁-C₆alkylene-SO₂O⁻; C₁-C₆alkylene-COOH; C₁-C₆alkylene-PO(OH)₂; C₁-C₆alkylene-NR′_{d}R′ₑ; C₁-C₆alkylene-N⁺R′_{d}R′ₑR′_{f}; C₁-C₆alkylene-O—C₁-C₆alkylene-OH; C₁-C₆alkylene-phenyl wherein the phenyl may be substituted by a hydroxyl or a C₁-C₆alkoxy group;
the group: 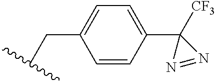 [SMILES: *Cc1ccc(C2(C(F)(F)F)N=N2)cc1]
or R′ₐ and R′_{b} form with the nitrogen atom carrying them a cycle B₃,
or R′ₐ, R′_{b} and R′_{c} form with the nitrogen atom carrying them a bridged C₃-C₈heterocycloalkyl,
R′_{c}, R′_{d}, R′ₑ, R′_{f}, independently of one another, represents a hydrogen or a linear or branched C₁-C₆alkyl group,
or R′_{d} and R′ₑ form with the nitrogen atom carrying them a cycle B₄,
or R′_{d}, R′ₑ and R′_{f} form with the nitrogen atom carrying them a bridged C₃-C₈heterocycloalkyl,
m=0, 1 or 2,
p=1, 2, 3 or 4,
B₃ and B₄, independently of one another, represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen, sulphur and nitrogen, (iii) be substituted by one or two groups selected from: fluorine, bromine, chlorine, linear or branched C₁-C₆alkyl, hydroxyl, —NH₂, oxo or piperidinyl; preferably
G is selected from the group consisting of:—C(O)OH, —C(O)OR_{G3}, —C(O)NR_{G1}R_{G2}, —C(O)R_{G2}, —NR_{G1}C(O)R_{G2}, —NR_{G1}C(O)NR_{G1}R_{G2}, —OC(O)NR_{G1}R_{G2}, —NR_{G1}C(O)OR_{G3}, —C(═NOR_{G1})NR_{G1}R_{G2}, —NR_{G1}C(═NCN)NR_{G1}R_{G2}, —NR_{G1}S(O)₂NR_{G1}R_{G2}, —S(O)₂R_{G3}, —S(O)₂NR_{G1}R_{G2}, —NR_{G1}S(O)₂R_{G2}, —NR_{G1}C(═NR_{G2})NR_{G1}R_{G2}, —C(═S)NR_{G1}R_{G2}, —C(═NR_{G1})NR_{G1}R_{G2}, halogen, —NO₂, and —CN;
R₇ represents a group selected from: linear or branched C₁-C₆alkyl group; (C₃-C₆) cycloalkylene-R₈; or: 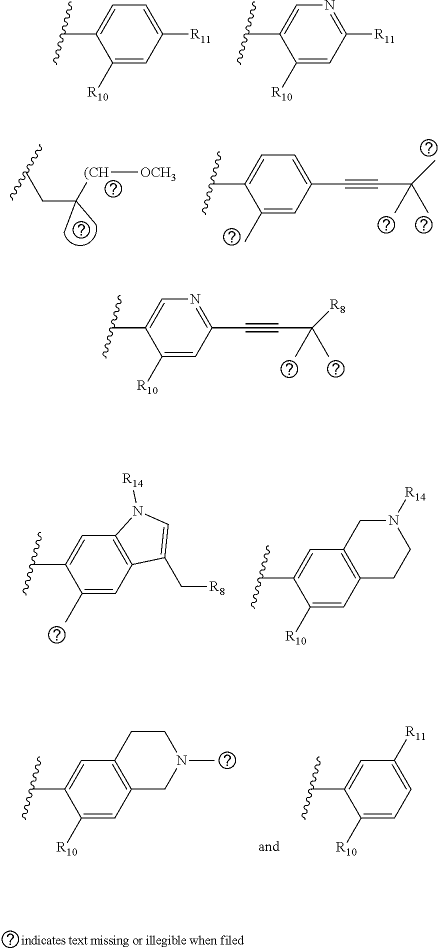
wherein Cy represents a C₃-C₈cycloalkyl; or
R₇ represents a group selected from:

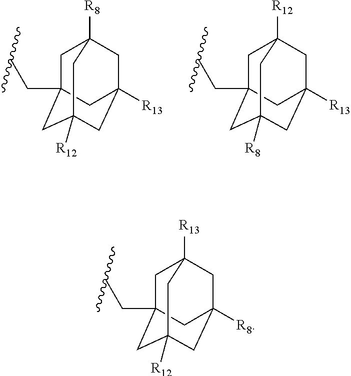

47-49. (canceled)

50. The antibody-drug conjugate of claim 38, wherein D comprises a compound of formula (IB), (IC), (IIB) or (IIC): 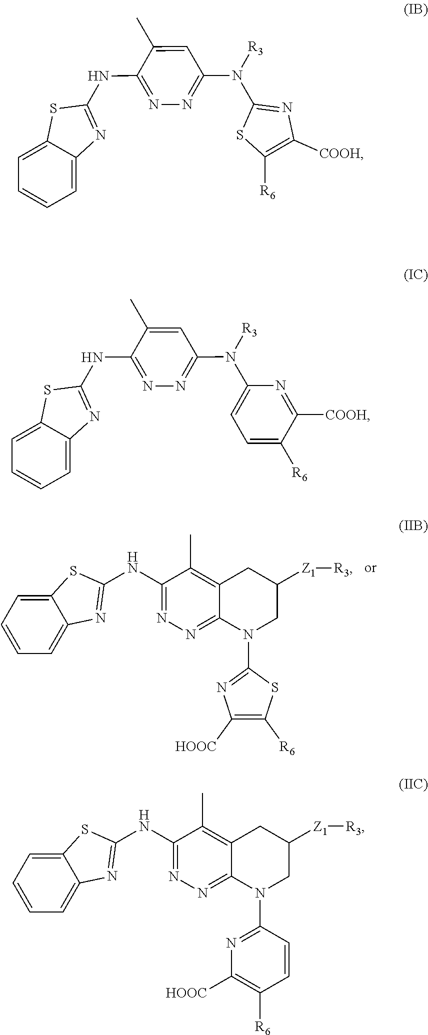
or an enantiomer, a diastereoisomer, and/or a pharmaceutically acceptable salt thereof, wherein:
for formula (IB) or (IC), R₃ represents a group selected from: hydrogen; linear or branched C₁-C₆alkyl; —X₁—NRₐR_{b}; —X₁—N⁺RₐR_{b}R_{c}; and —X₁—O—R_{c};
for formula (IIB) or (IIC), Z₁ represents a bond, and R₃ represents hydrogen; or Z₁ represents —O—, and R³ represents —X₁—NRₐR_{b},
Rₐ and R_{b} independently of one another represent a group selected from: hydrogen; linear or branched C₁-C₆alkyl optionally substituted by one or two hydroxyl groups; and C₁-C₆alkylene-SO₂O⁻,
R_{c} represents a hydrogen or a linear or branched C₁-C₆alkyl group
R₆ represents —X₂—O—R₇ or an heteroarylene-R₇ group optionally substituted by a linear or branched C₁-C₆alkyl group,
R₇ represents a group selected from: 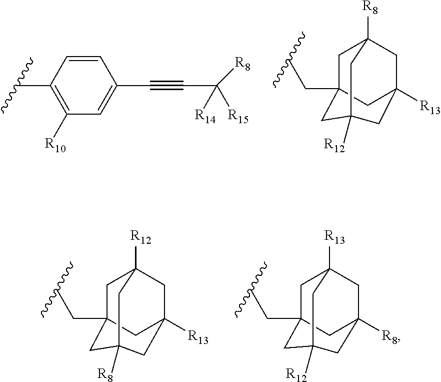
R₈ represents a group selected from: —NR′ₐR′_{b}; —O—X′₂—NR′ₐR′_{b}; and —X′₂—NR′ₐR′_{b},
R₁₀ represents fluorine,
R₁₂ and R₁₃, independently of one another, represent a hydrogen atom or a methyl group,
R₁₄ and R₁₅, independently of one another, represent a hydrogen or a methyl group,
X₁ and X₂ independently of one another, represent a linear or branched C₁-C₆alkylene group optionally substituted by one or two groups selected from trifluoromethyl, hydroxyl, halogen, C₁-C₆alkoxy,
X′₂ represents a linear or branched C₁-C₆alkylene,
R′ₐ and R′_{b} independently of one another, represent a group selected from: hydrogen; linear or branched C₁-C₆alkyl optionally substituted by one or two hydroxyl or C₁-C₆alkoxy groups; C₁-C₆alkylene-NR′ₐR′ₑ;
or R′ₐ and R′_{b} form with the nitrogen atom carrying them a cycle B₃,
R′_{d}, R′ₑ independently of one another, represents a hydrogen or a linear or branched C₁-C₆alkyl group,
B₃ represents a C₃-C₈heterocycloalkyl group, which group can: (i) be a mono- or bi-cyclic group, wherein bicyclic group includes fused, bridged or spiro ring system, (ii) can contain, in addition to the nitrogen atom, one or two hetero atoms selected independently from oxygen and nitrogen, (iii) be substituted by one or two groups selected from: fluorine, bromine, chlorine, linear or branched C₁-C₆alkyl, hydroxyl, and oxo.

51. The antibody-drug conjugate of claim 50, wherein:
(1) R₇ represents the following group: 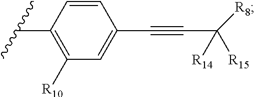
or
(2) R₇ represents a group selected from: 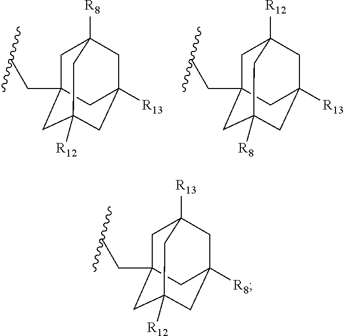
optionally
wherein
R₈ represents a group selected from: 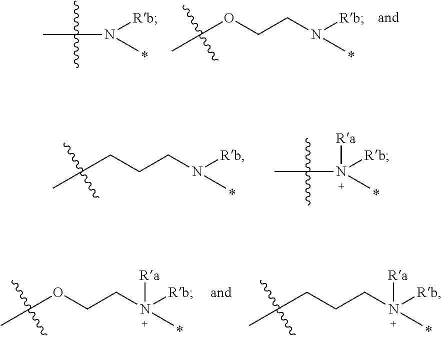
wherein [symbol] represents a bond to the linker.

52-53. (canceled)

54. The antibody-drug conjugate of claim 51, wherein B₃ represents a C₃-C₈heterocycloalkyl group selected from a pyrrolidinyl group, a piperidinyl group, a piperazinyl group, a morpholinyl group, an azepanyl group, and a 4,4-difluoropiperidin-1-yl group.

55. The antibody-drug conjugate of claim 38, wherein:
(1) D represents any one of the following attached to L: 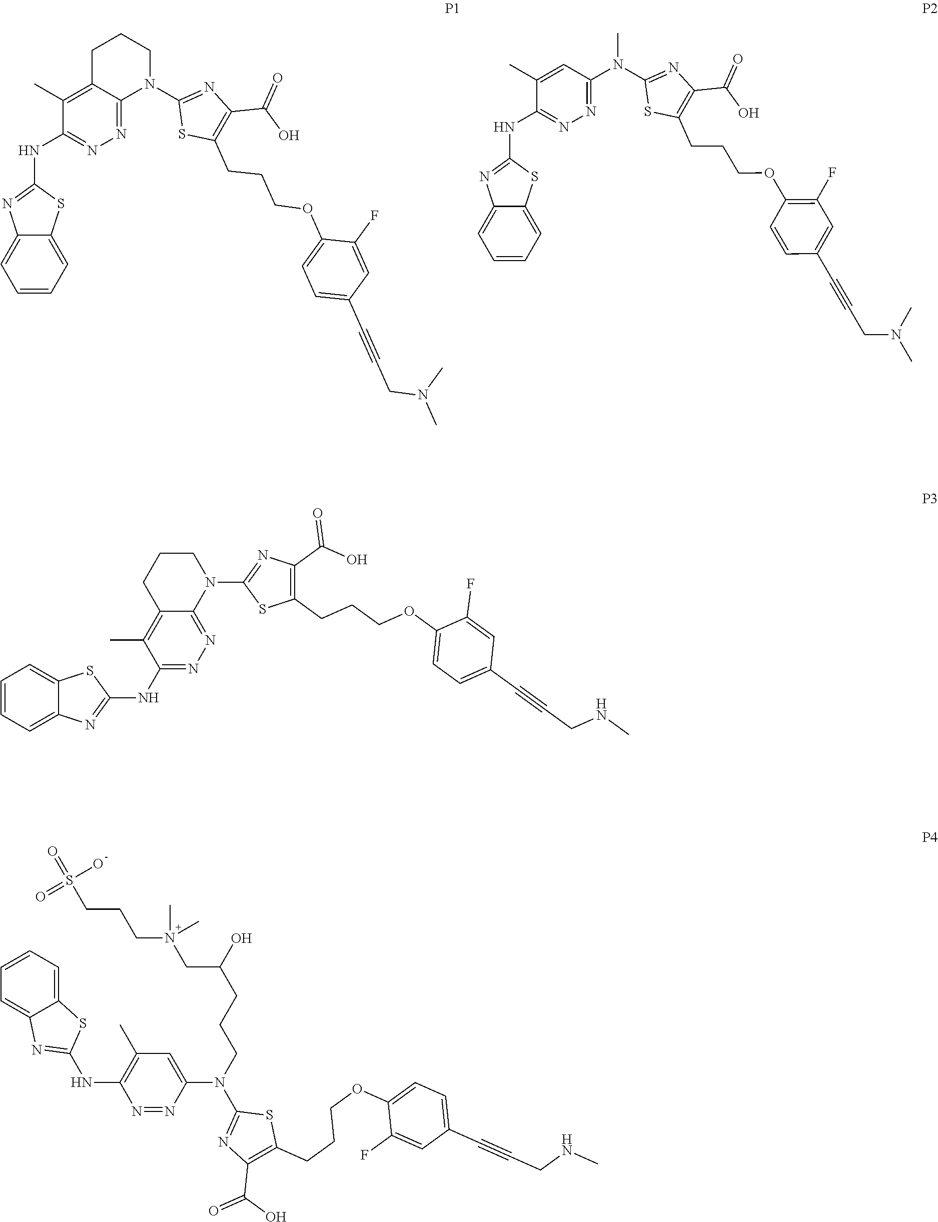 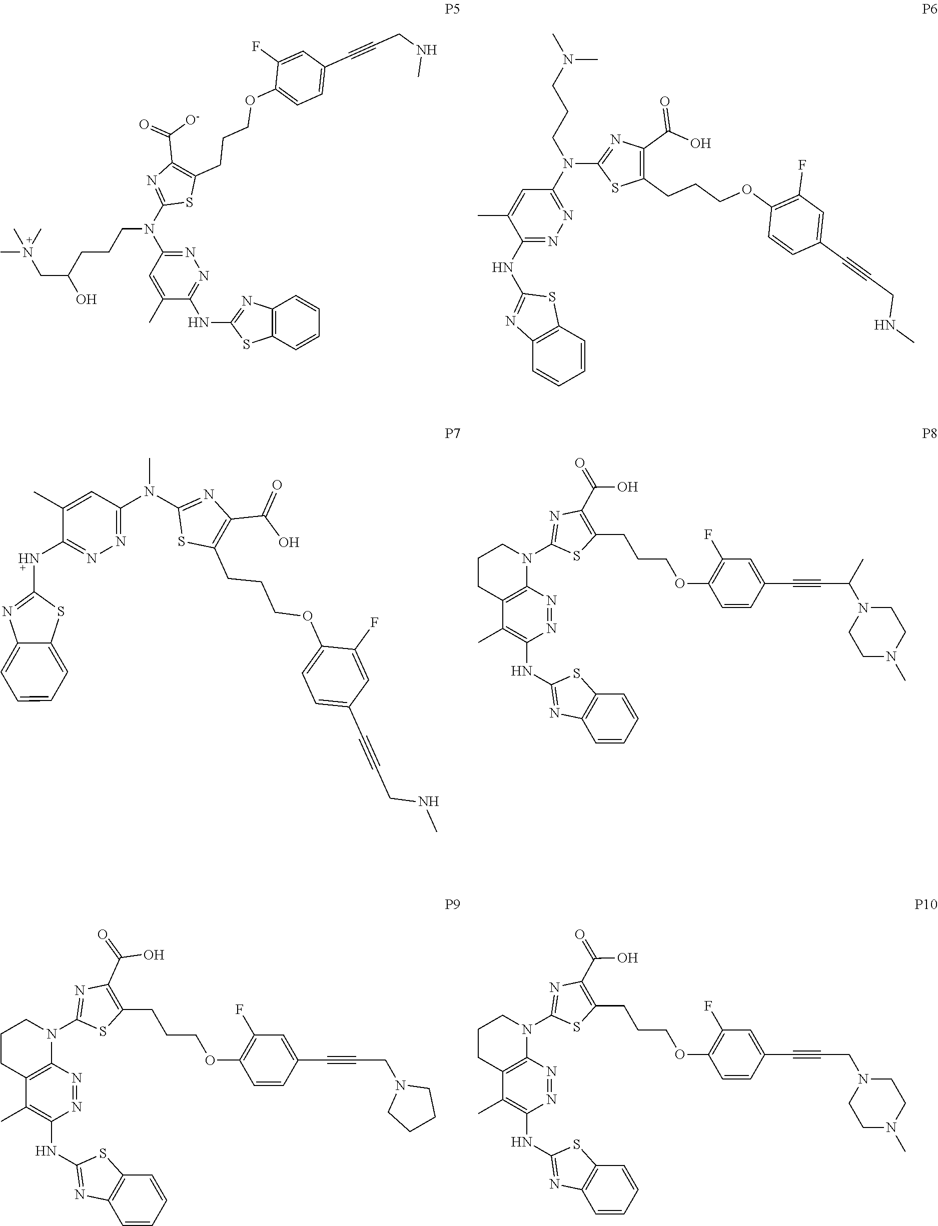 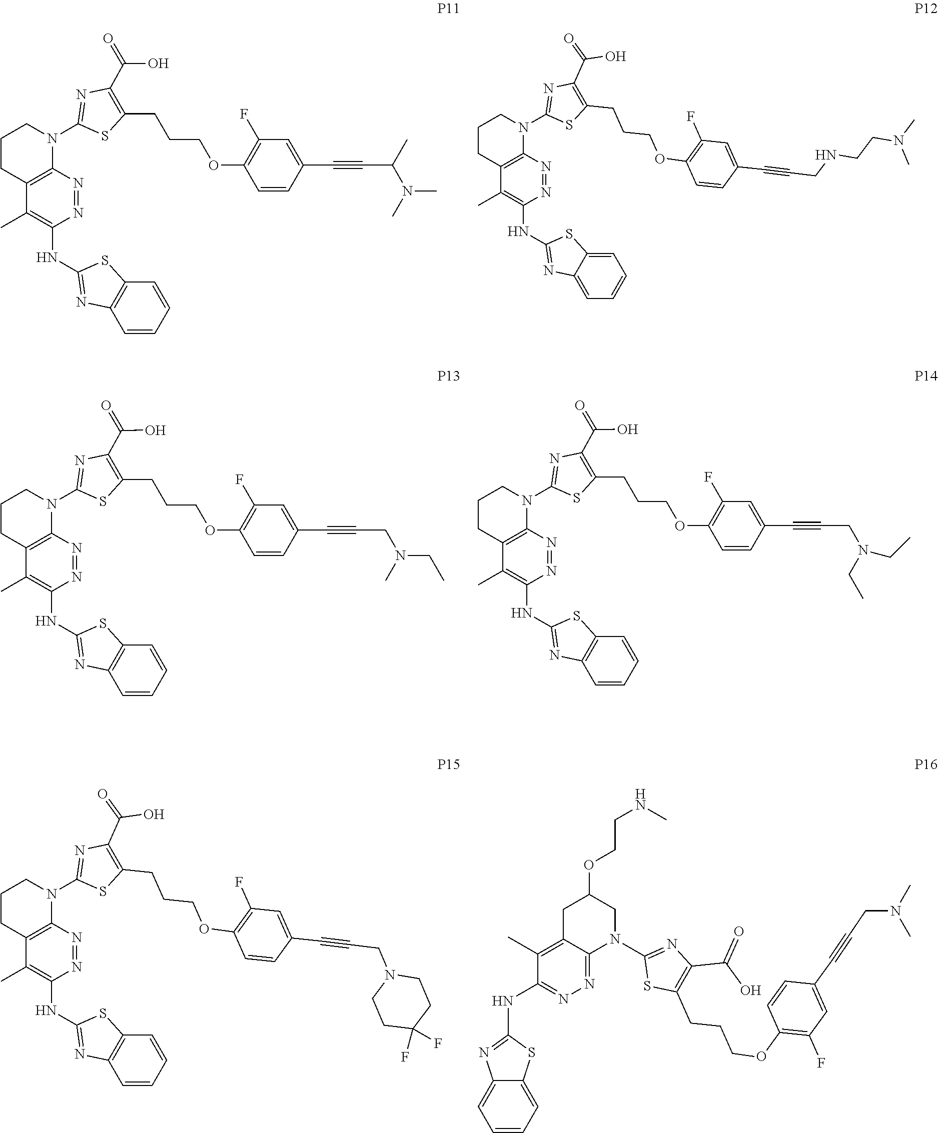 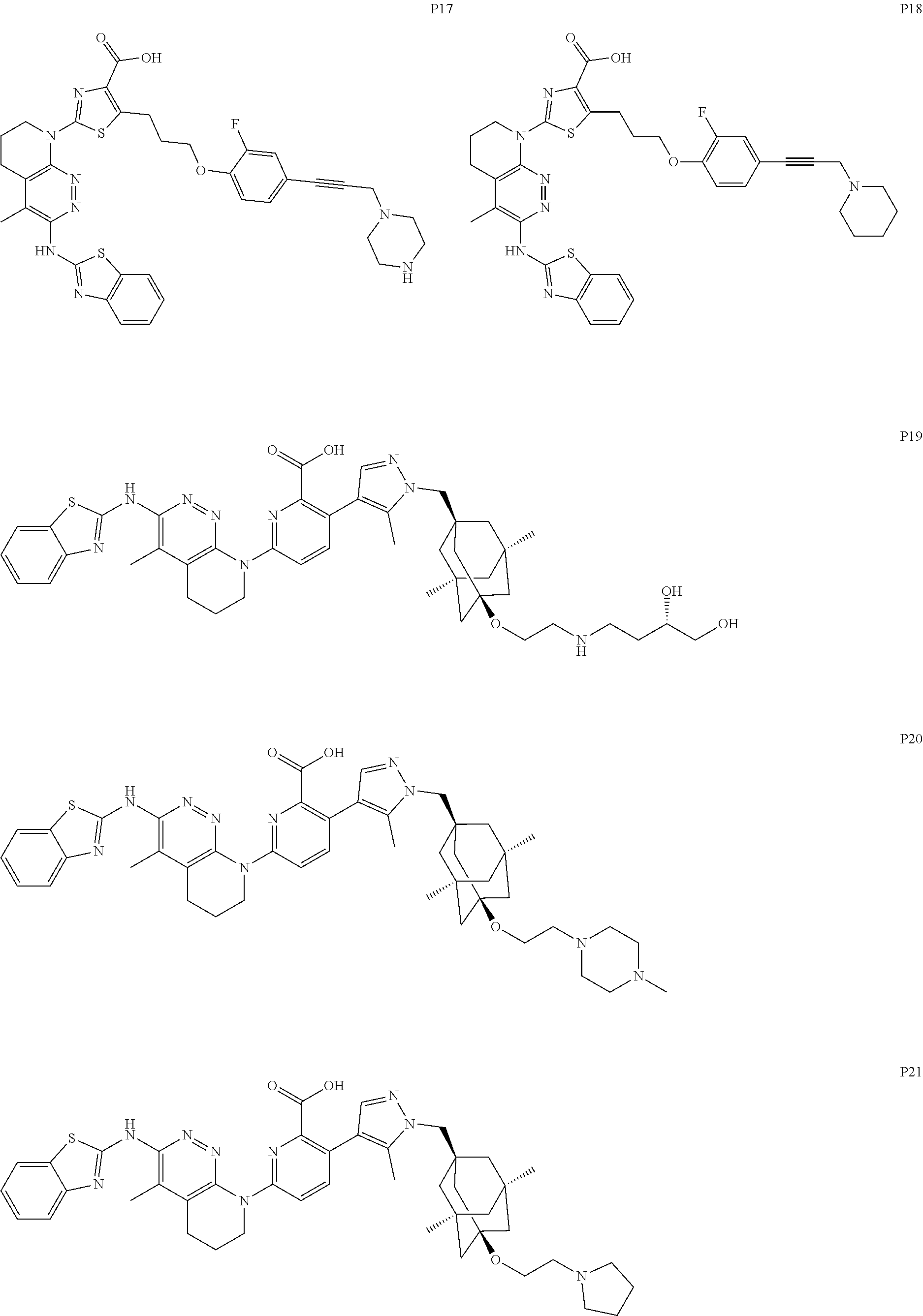 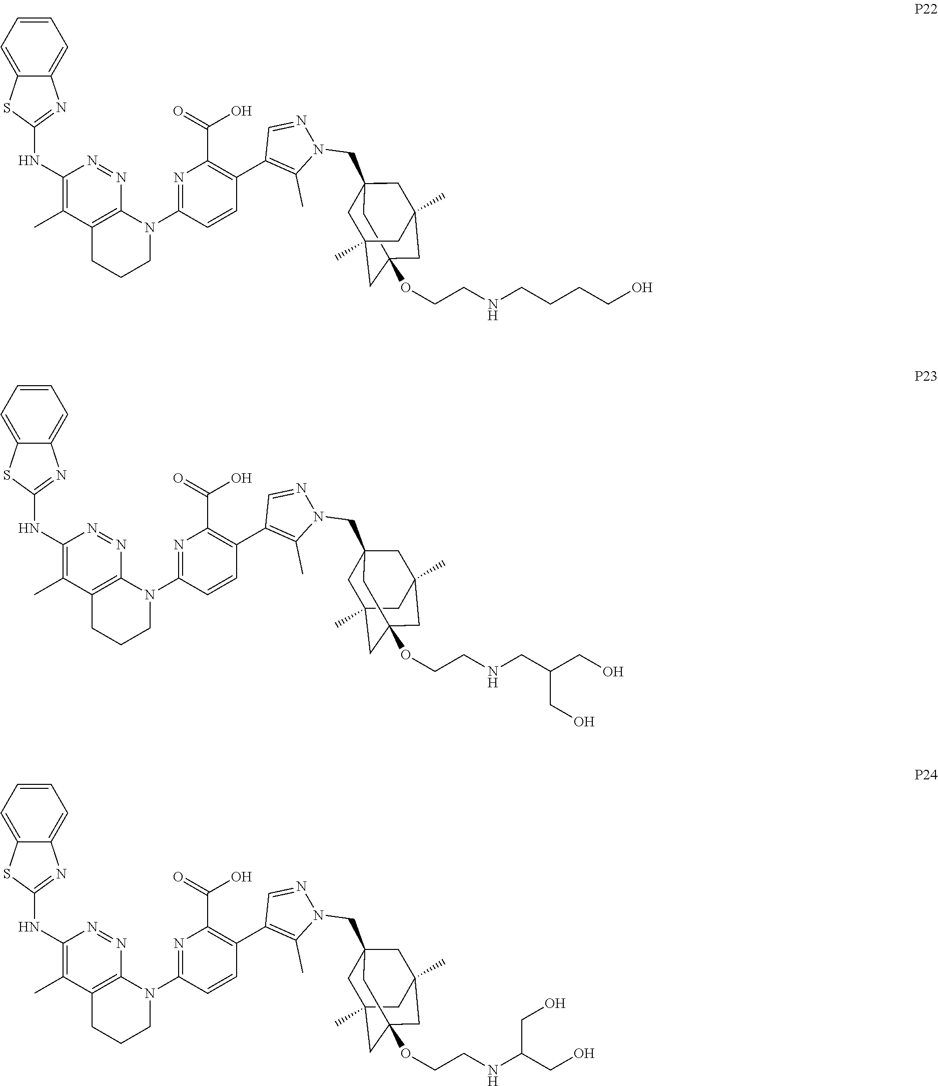 [SMILES: Cc1c(Nc2nc3ccccc3s2)nnc2c1CCCN2c1ccc(-c2cnn(C[C@]34C[C@]5(C)C[C@](C)(C3)C[C@@](OCCNCCCCO)(C5)C4)c2C)c(C(=O)O)n1 ; Cc1c(Nc2nc3ccccc3s2)nnc2c1CCCN2c1ccc(-c2cnn(C[C@]34C[C@]5(C)C[C@](C)(C3)C[C@@](OCCNCC(CO)CO)(C5)C4)c2C)c(C(=O)O)n1 ; Cc1c(Nc2nc3ccccc3s2)nnc2c1CCCN2c1ccc(-c2cnn(C[C@]34C[C@]5(C)C[C@](C)(C3)C[C@@](OCCNC(CO)CO)(C5)C4)c2C)c(C(=O)O)n1] 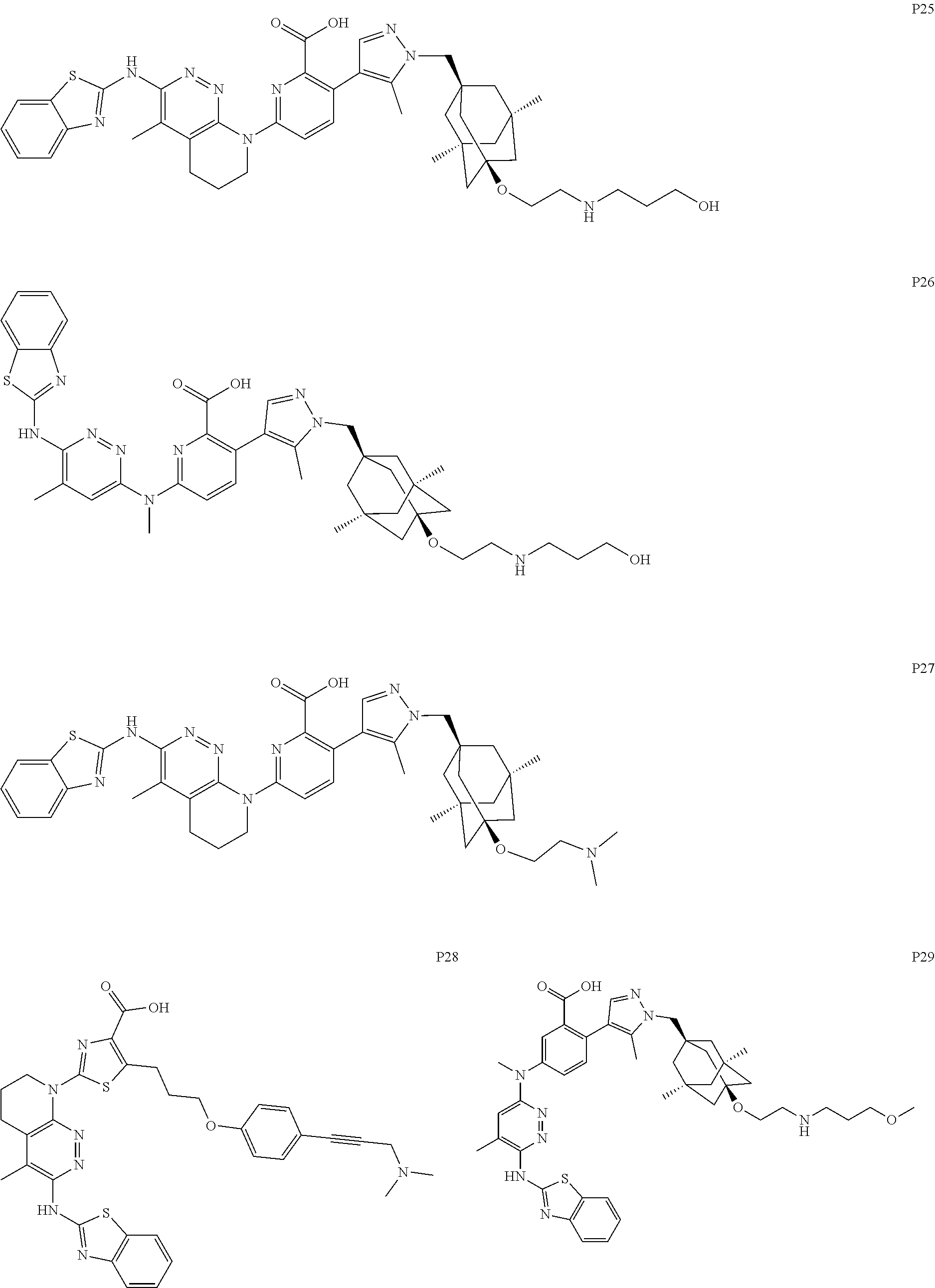 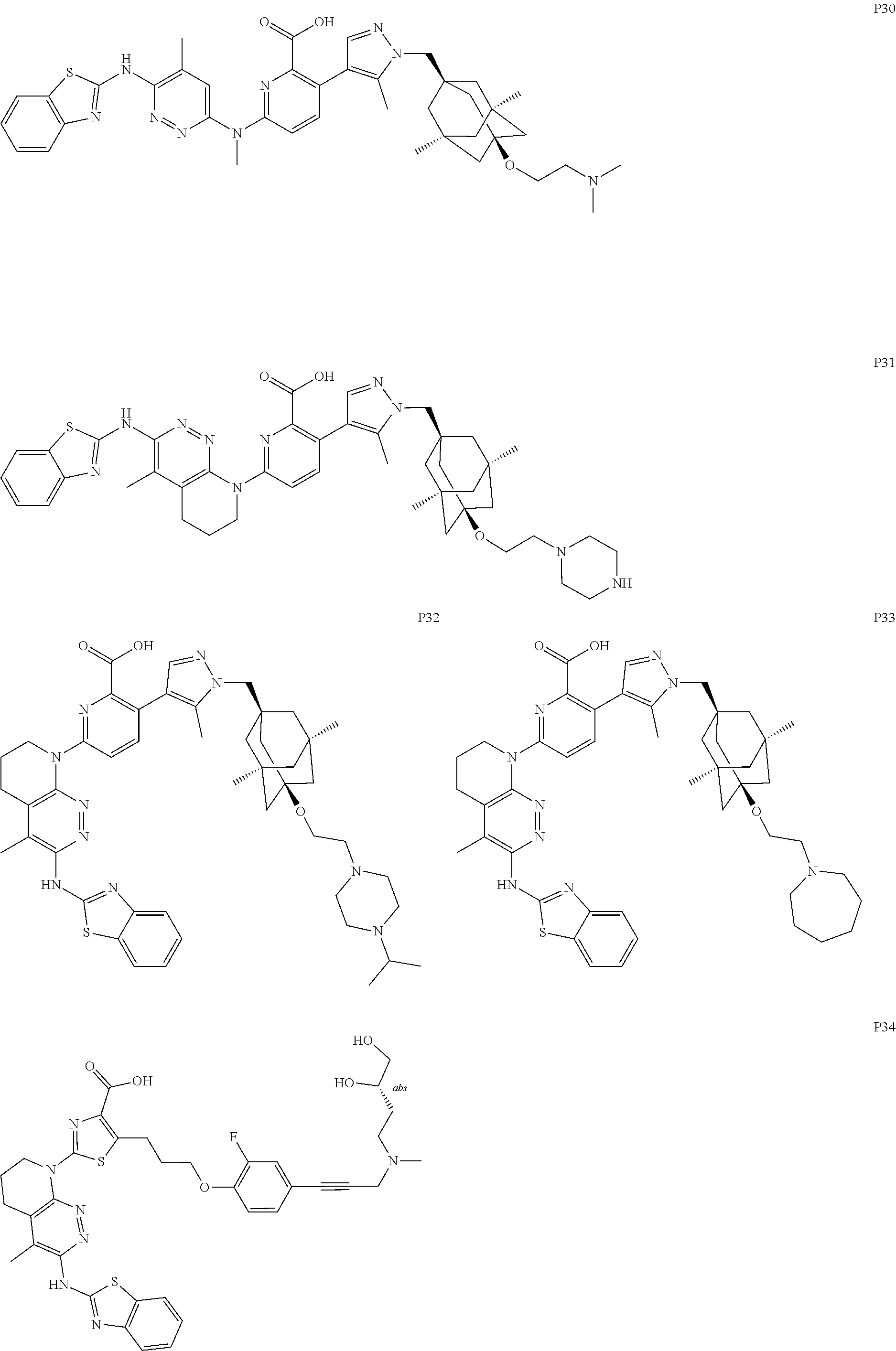 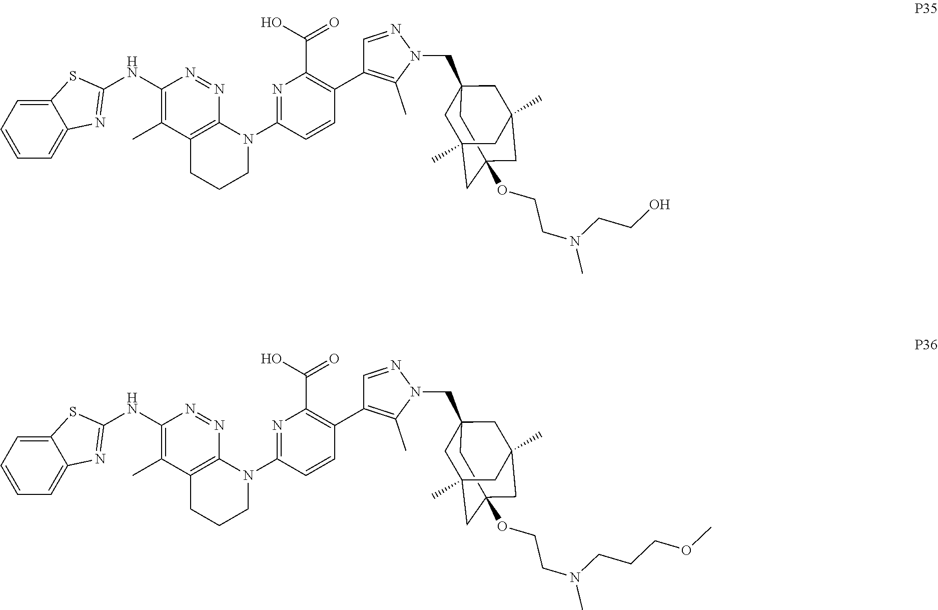 [SMILES: Cc1c(Nc2nc3ccccc3s2)nnc2c1CCCN2c1ccc(-c2cnn(C[C@]34C[C@]5(C)C[C@](C)(C3)C[C@@](OCCN(C)CCO)(C5)C4)c2C)c(C(=O)O)n1 ; COCCCN(C)CCO[C@]12C[C@@]3(C)C[C@](C)(C[C@](Cn4ncc(-c5ccc(N6CCCc7c6nnc(Nc6nc8ccccc8s6)c7C)nc5C(=O)O)c4C)(C3)C1)C2] 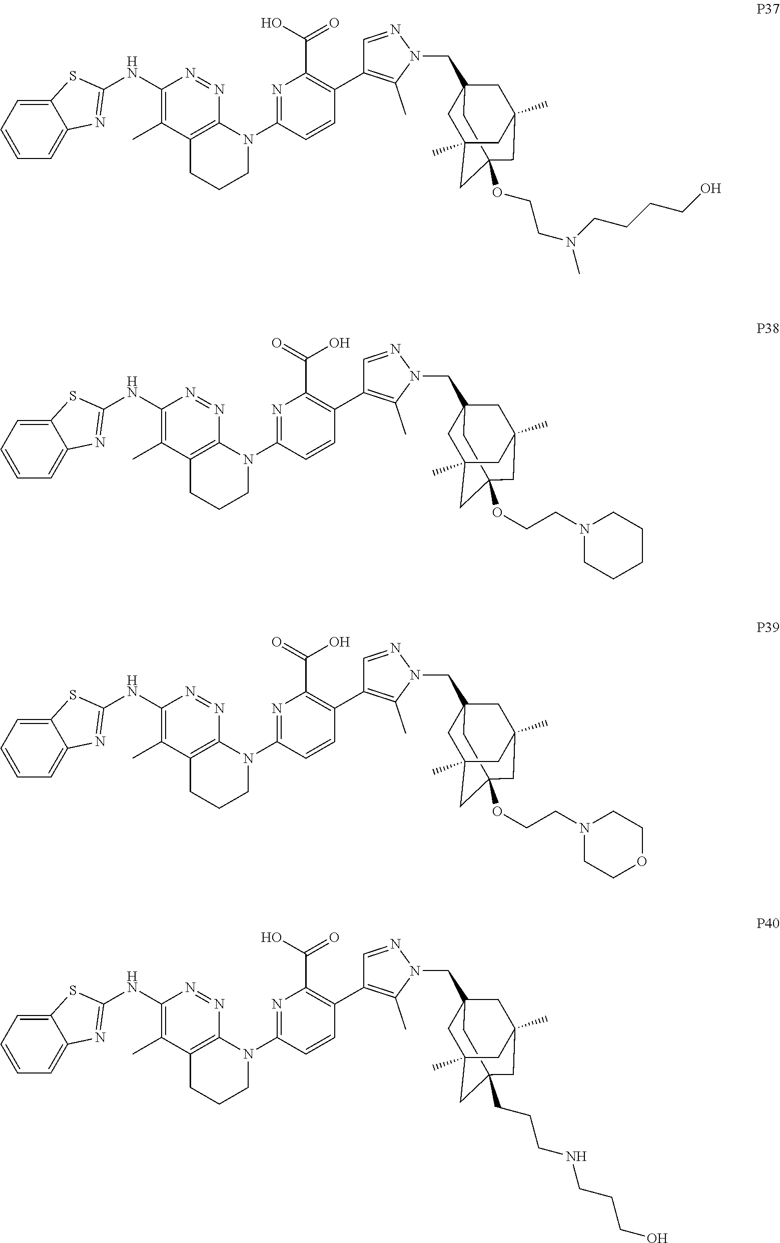 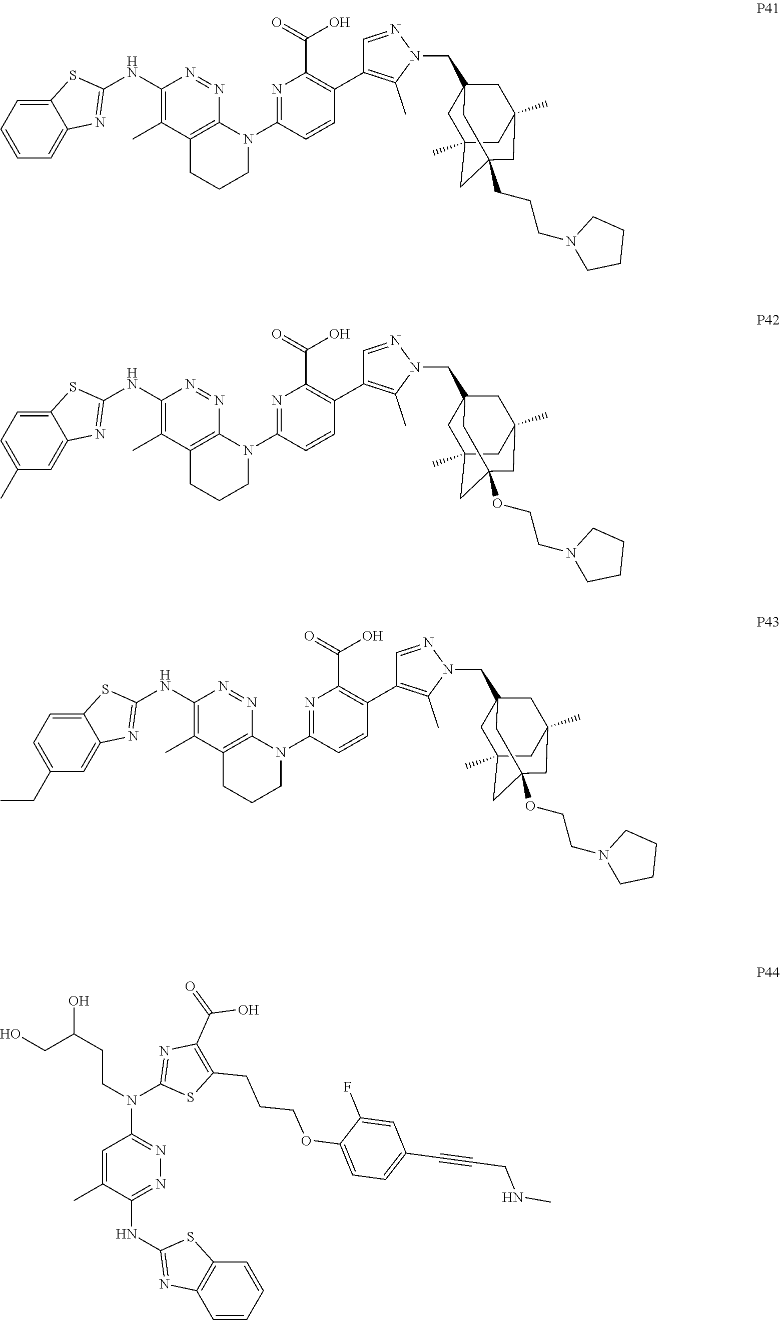 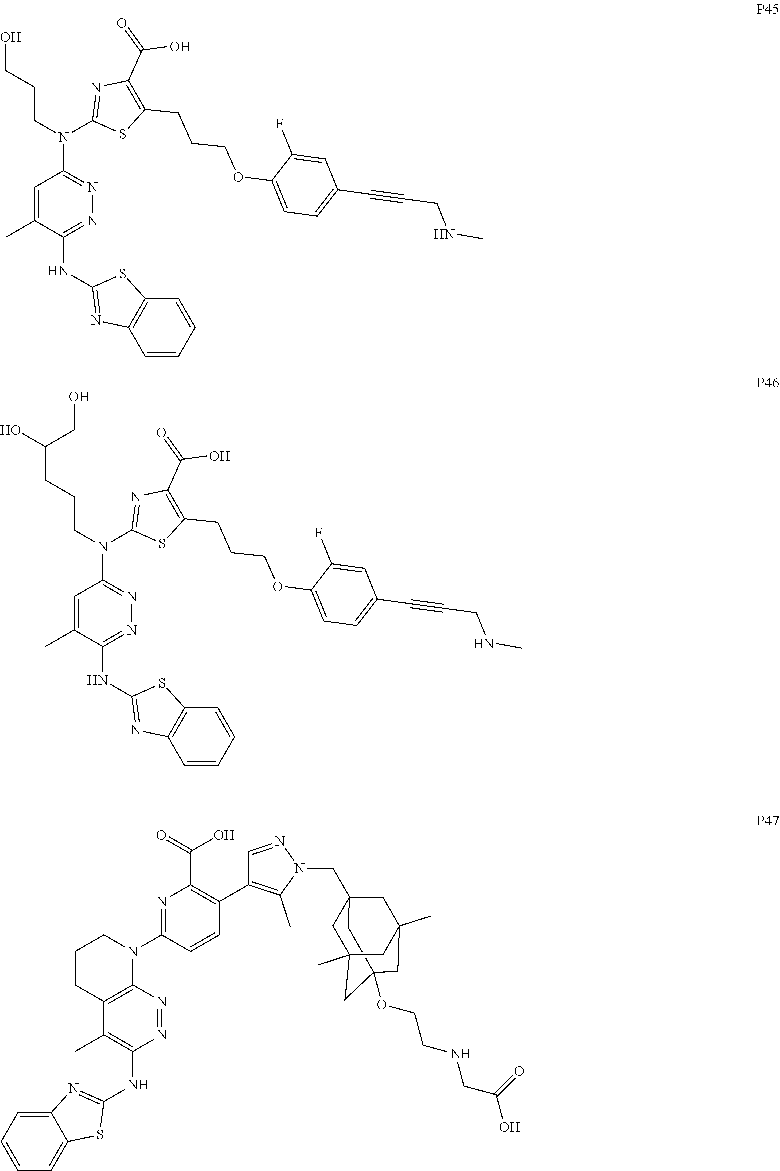 [SMILES: CNCC#Cc1ccc(OCCCc2sc(N(CCCO)c3cc(C)c(Nc4nc5ccccc5s4)nn3)nc2C(=O)O)c(F)c1 ; CNCC#Cc1ccc(OCCCc2sc(N(CCCC(O)CO)c3cc(C)c(Nc4nc5ccccc5s4)nn3)nc2C(=O)O)c(F)c1 ; Cc1c(Nc2nc3ccccc3s2)nnc2c1CCCN2c1ccc(-c2cnn(CC34CC5(C)CC(C)(C3)CC(OCCNCC(=O)O)(C5)C4)c2C)c(C(=O)O)n1] 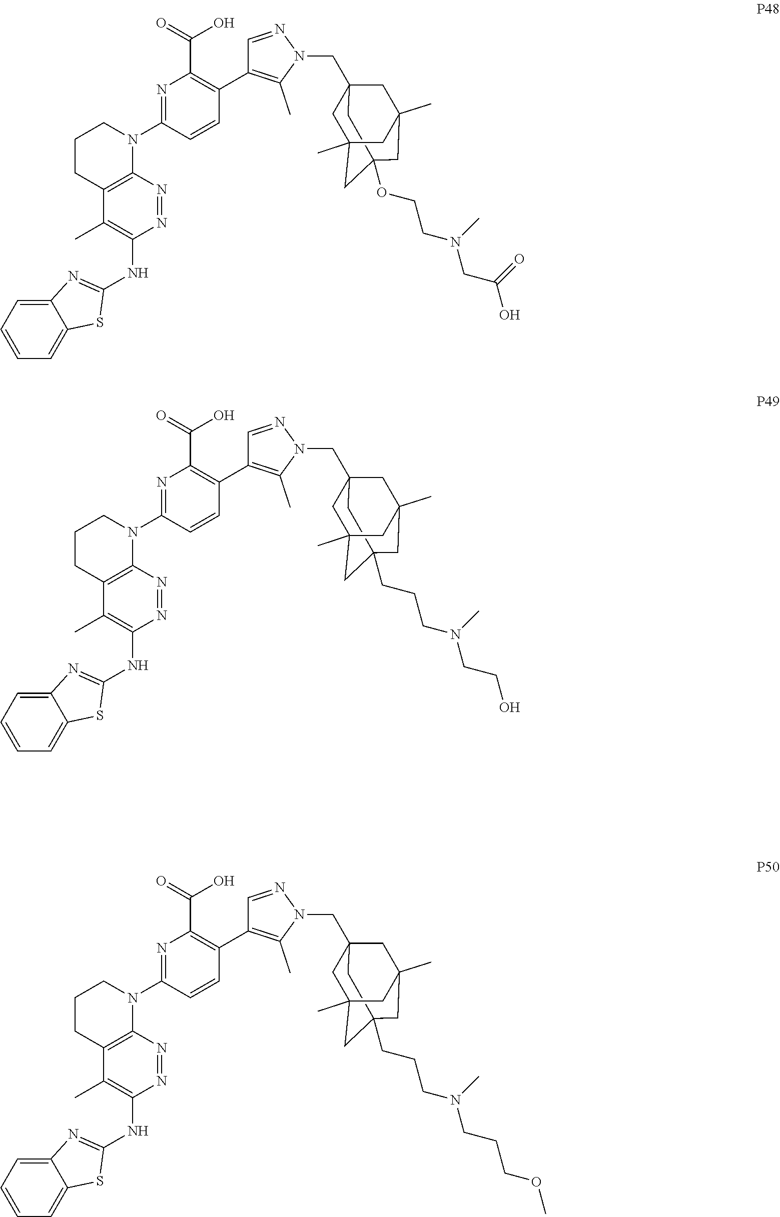 [SMILES: Cc1c(Nc2nc3ccccc3s2)nnc2c1CCCN2c1ccc(-c2cnn(CC34CC5(C)CC(C)(C3)CC(OCCN(C)CC(=O)O)(C5)C4)c2C)c(C(=O)O)n1 ; Cc1c(Nc2nc3ccccc3s2)nnc2c1CCCN2c1ccc(-c2cnn(CC34CC5(C)CC(C)(CC(CCCN(C)CCO)(C5)C3)C4)c2C)c(C(=O)O)n1 ; COCCCN(C)CCCC12CC3(C)CC(C)(C1)CC(Cn1ncc(-c4ccc(N5CCCc6c5nnc(Nc5nc7ccccc7s5)c6C)nc4C(=O)O)c1C)(C3)C2] 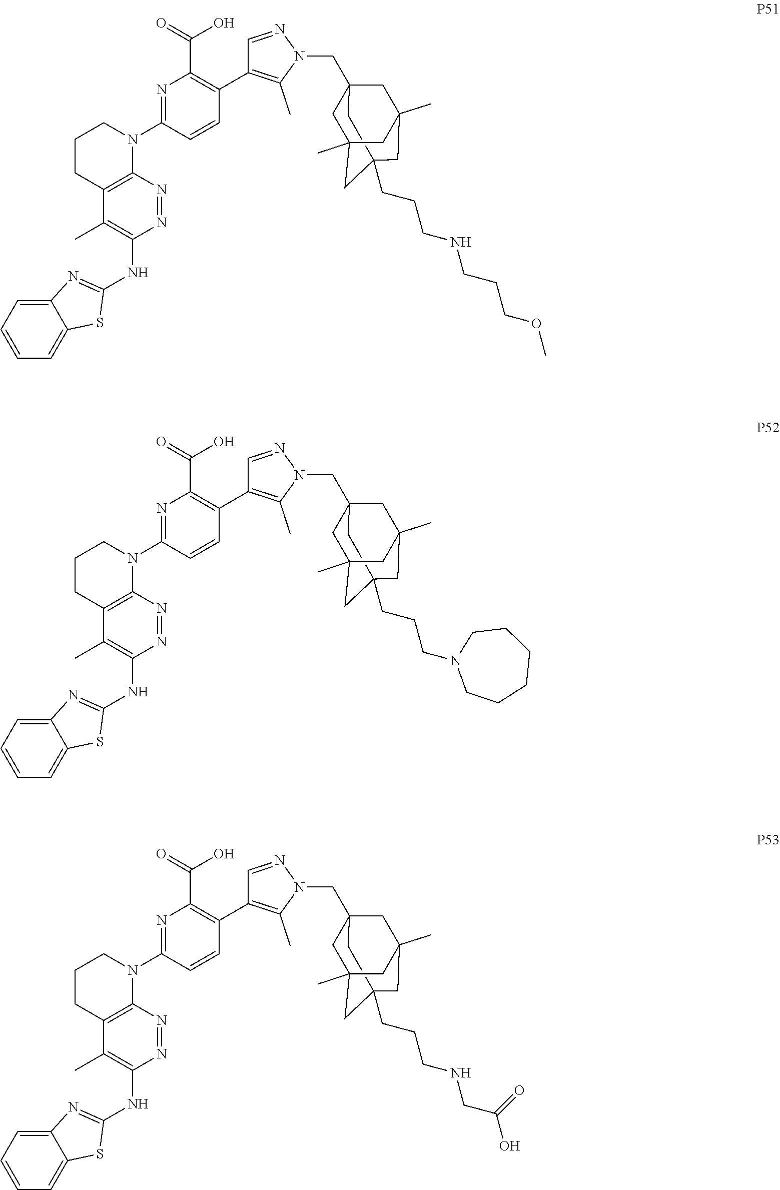 [SMILES: COCCCNCCCC12CC3(C)CC(C)(C1)CC(Cn1ncc(-c4ccc(N5CCCc6c5nnc(Nc5nc7ccccc7s5)c6C)nc4C(=O)O)c1C)(C3)C2 ; Cc1c(Nc2nc3ccccc3s2)nnc2c1CCCN2c1ccc(-c2cnn(CC34CC5(C)CC(C)(CC(CCCN6CCCCCC6)(C5)C3)C4)c2C)c(C(=O)O)n1 ; Cc1c(Nc2nc3ccccc3s2)nnc2c1CCCN2c1ccc(-c2cnn(CC34CC5(C)CC(C)(CC(CCCNCC(=O)O)(C5)C3)C4)c2C)c(C(=O)O)n1] 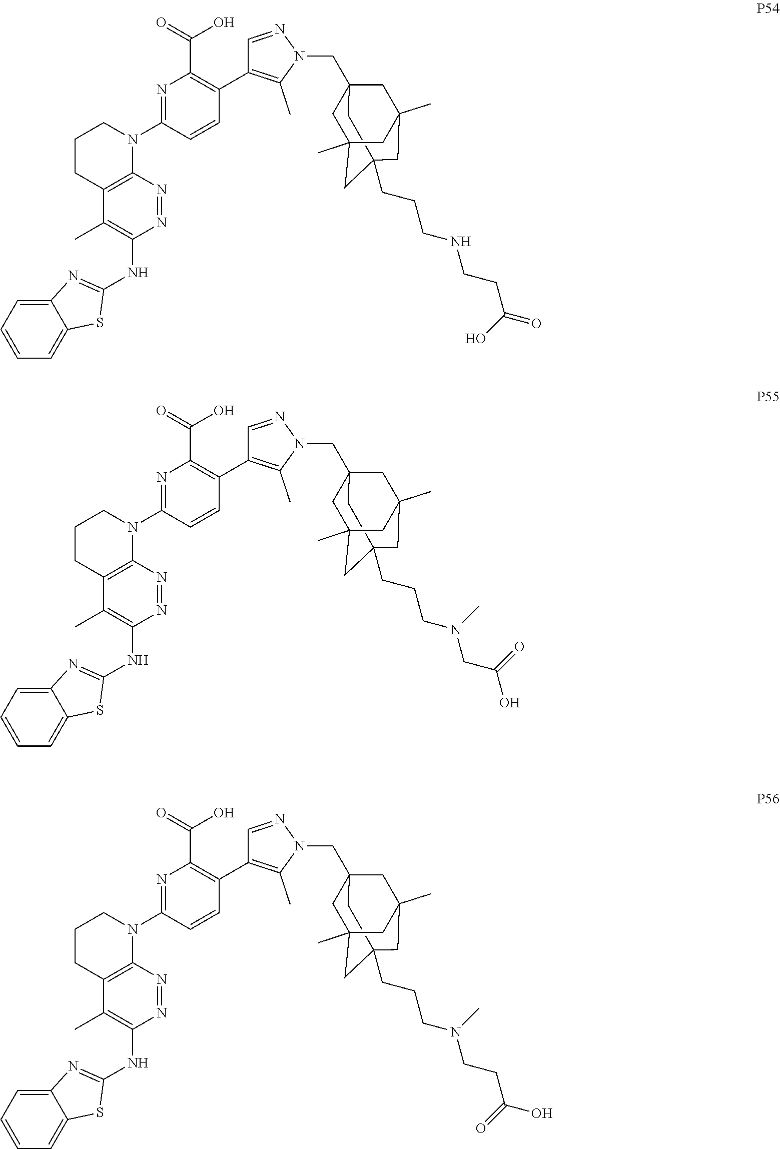 [SMILES: Cc1c(Nc2nc3ccccc3s2)nnc2c1CCCN2c1ccc(-c2cnn(CC34CC5(C)CC(C)(CC(CCCNCCC(=O)O)(C5)C3)C4)c2C)c(C(=O)O)n1 ; Cc1c(Nc2nc3ccccc3s2)nnc2c1CCCN2c1ccc(-c2cnn(CC34CC5(C)CC(C)(CC(CCCN(C)CC(=O)O)(C5)C3)C4)c2C)c(C(=O)O)n1 ; Cc1c(Nc2nc3ccccc3s2)nnc2c1CCCN2c1ccc(-c2cnn(CC34CC5(C)CC(C)(CC(CCCN(C)CCC(=O)O)(C5)C3)C4)c2C)c(C(=O)O)n1] 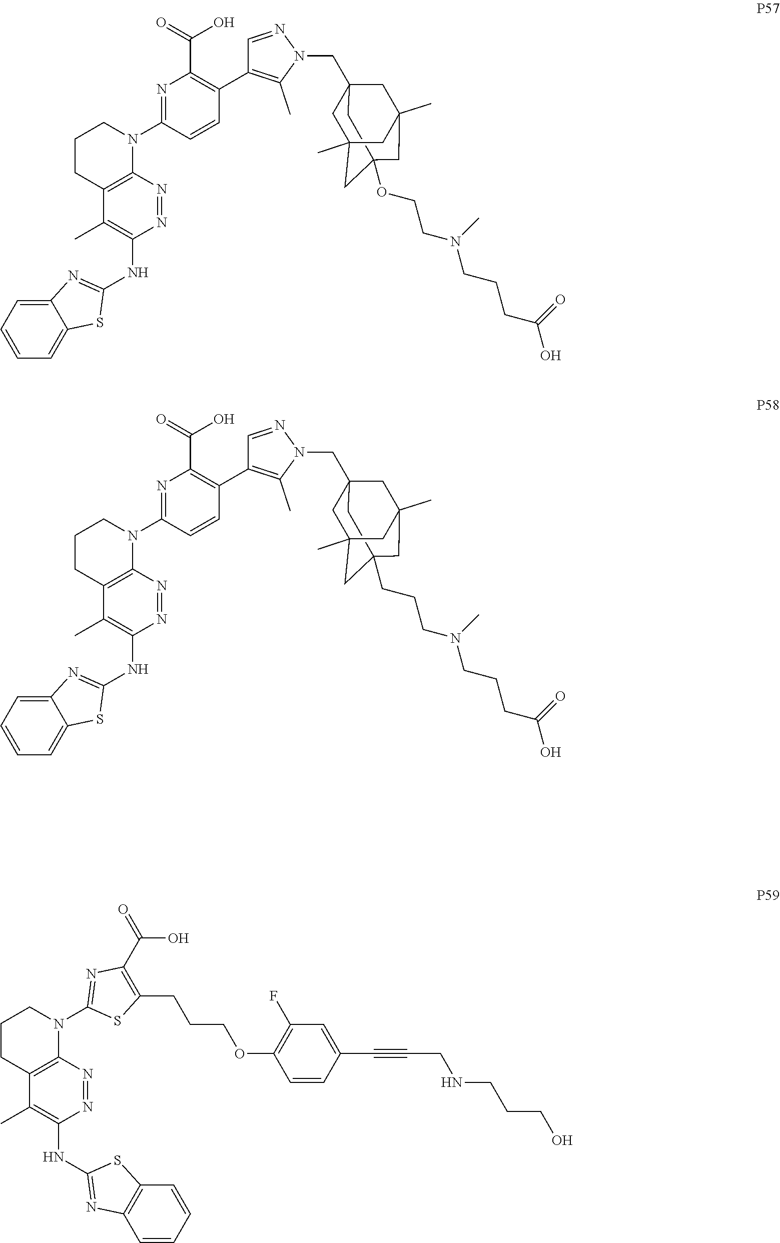 [SMILES: Cc1c(Nc2nc3ccccc3s2)nnc2c1CCCN2c1ccc(-c2cnn(CC34CC5(C)CC(C)(C3)CC(OCCN(C)CCCC(=O)O)(C5)C4)c2C)c(C(=O)O)n1 ; Cc1c(Nc2nc3ccccc3s2)nnc2c1CCCN2c1ccc(-c2cnn(CC34CC5(C)CC(C)(CC(CCCN(C)CCCC(=O)O)(C5)C3)C4)c2C)c(C(=O)O)n1 ; Cc1c(Nc2nc3ccccc3s2)nnc2c1CCCN2c1nc(C(=O)O)c(CCCOc2ccc(C#CCNCCCO)cc2F)s1] 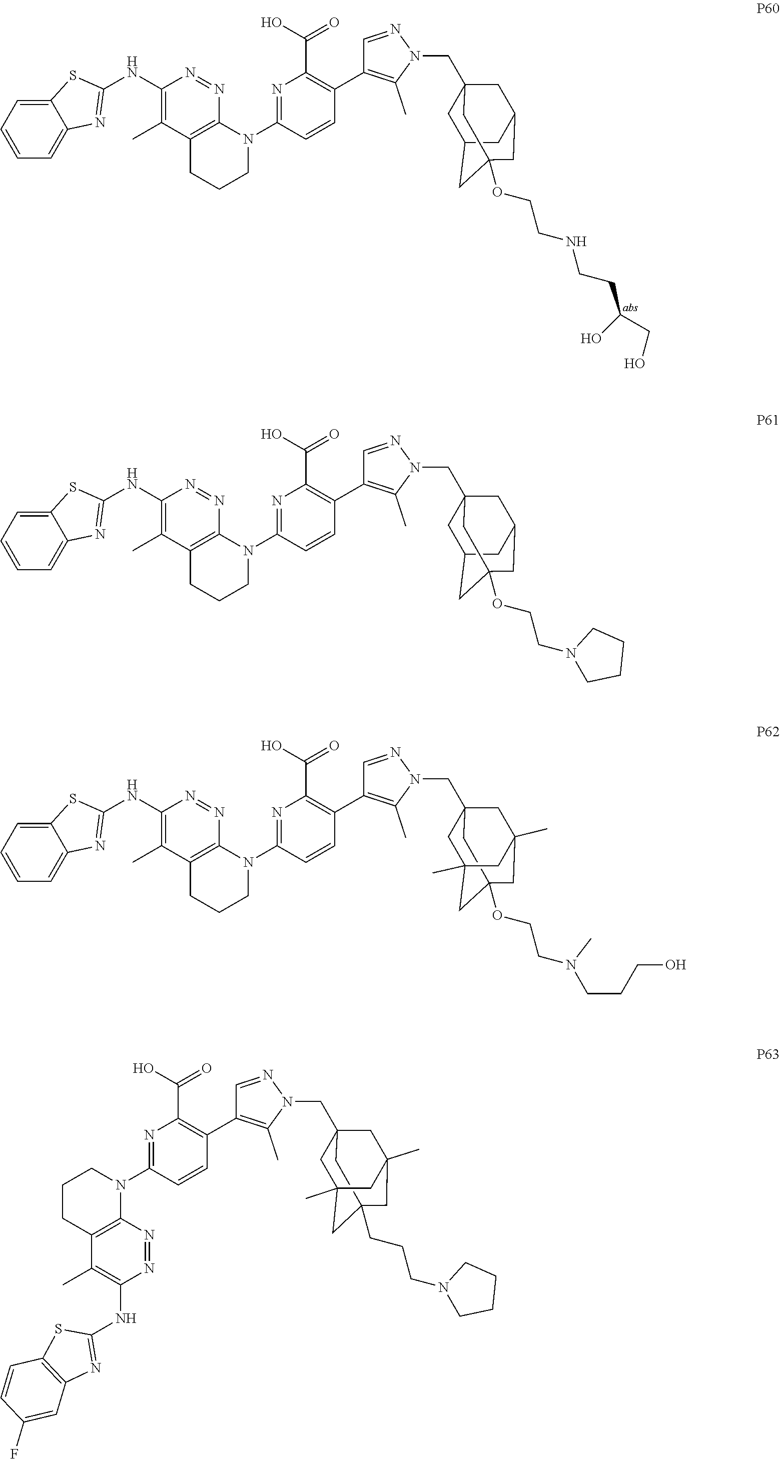 [SMILES: Cc1c(Nc2nc3ccccc3s2)nnc2c1CCCN2c1ccc(-c2cnn(CC34CC5CC(C3)CC(OCCNCC[C@H](O)CO)(C5)C4)c2C)c(C(=O)O)n1 ; Cc1c(Nc2nc3ccccc3s2)nnc2c1CCCN2c1ccc(-c2cnn(CC34CC5CC(C3)CC(OCCN3CCCC3)(C5)C4)c2C)c(C(=O)O)n1 ; Cc1c(Nc2nc3ccccc3s2)nnc2c1CCCN2c1ccc(-c2cnn(CC34CC5(C)CC(C)(C3)CC(OCCN(C)CCCO)(C5)C4)c2C)c(C(=O)O)n1 ; Cc1c(Nc2nc3cc(F)ccc3s2)nnc2c1CCCN2c1ccc(-c2cnn(CC34CC5(C)CC(C)(CC(CCCN6CCCC6)(C5)C3)C4)c2C)c(C(=O)O)n1] 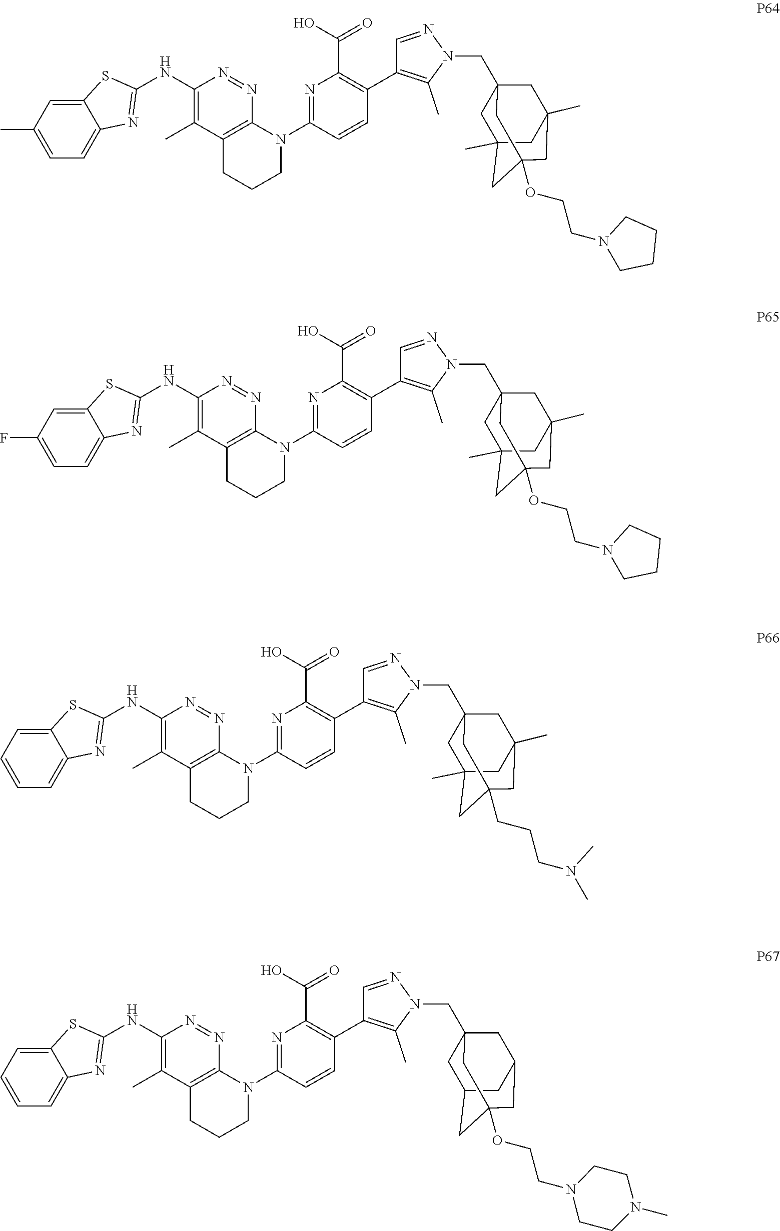 [SMILES: Cc1ccc2nc(Nc3nnc4c(c3C)CCCN4c3ccc(-c4cnn(CC56CC7(C)CC(C)(C5)CC(OCCN5CCCC5)(C7)C6)c4C)c(C(=O)O)n3)sc2c1 ; Cc1c(Nc2nc3ccc(F)cc3s2)nnc2c1CCCN2c1ccc(-c2cnn(CC34CC5(C)CC(C)(C3)CC(OCCN3CCCC3)(C5)C4)c2C)c(C(=O)O)n1 ; Cc1c(Nc2nc3ccccc3s2)nnc2c1CCCN2c1ccc(-c2cnn(CC34CC5(C)CC(C)(CC(CCCN(C)C)(C5)C3)C4)c2C)c(C(=O)O)n1 ; Cc1c(Nc2nc3ccccc3s2)nnc2c1CCCN2c1ccc(-c2cnn(CC34CC5CC(C3)CC(OCCN3CCN(C)CC3)(C5)C4)c2C)c(C(=O)O)n1] 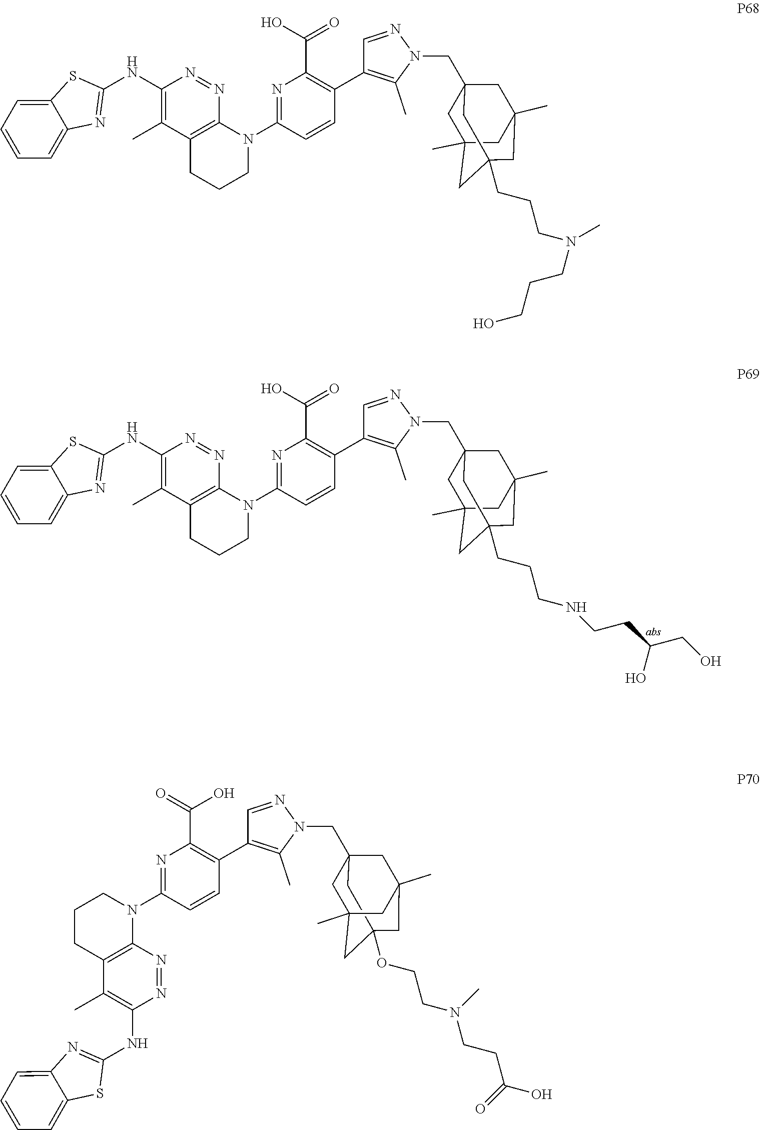 [SMILES: Cc1c(Nc2nc3ccccc3s2)nnc2c1CCCN2c1ccc(-c2cnn(CC34CC5(C)CC(C)(CC(CCCN(C)CCCO)(C5)C3)C4)c2C)c(C(=O)O)n1 ; Cc1c(Nc2nc3ccccc3s2)nnc2c1CCCN2c1ccc(-c2cnn(CC34CC5(C)CC(C)(CC(CCCNCC[C@H](O)CO)(C5)C3)C4)c2C)c(C(=O)O)n1 ; Cc1c(Nc2nc3ccccc3s2)nnc2c1CCCN2c1ccc(-c2cnn(CC34CC5(C)CC(C)(C3)CC(OCCN(C)CCC(=O)O)(C5)C4)c2C)c(C(=O)O)n1] 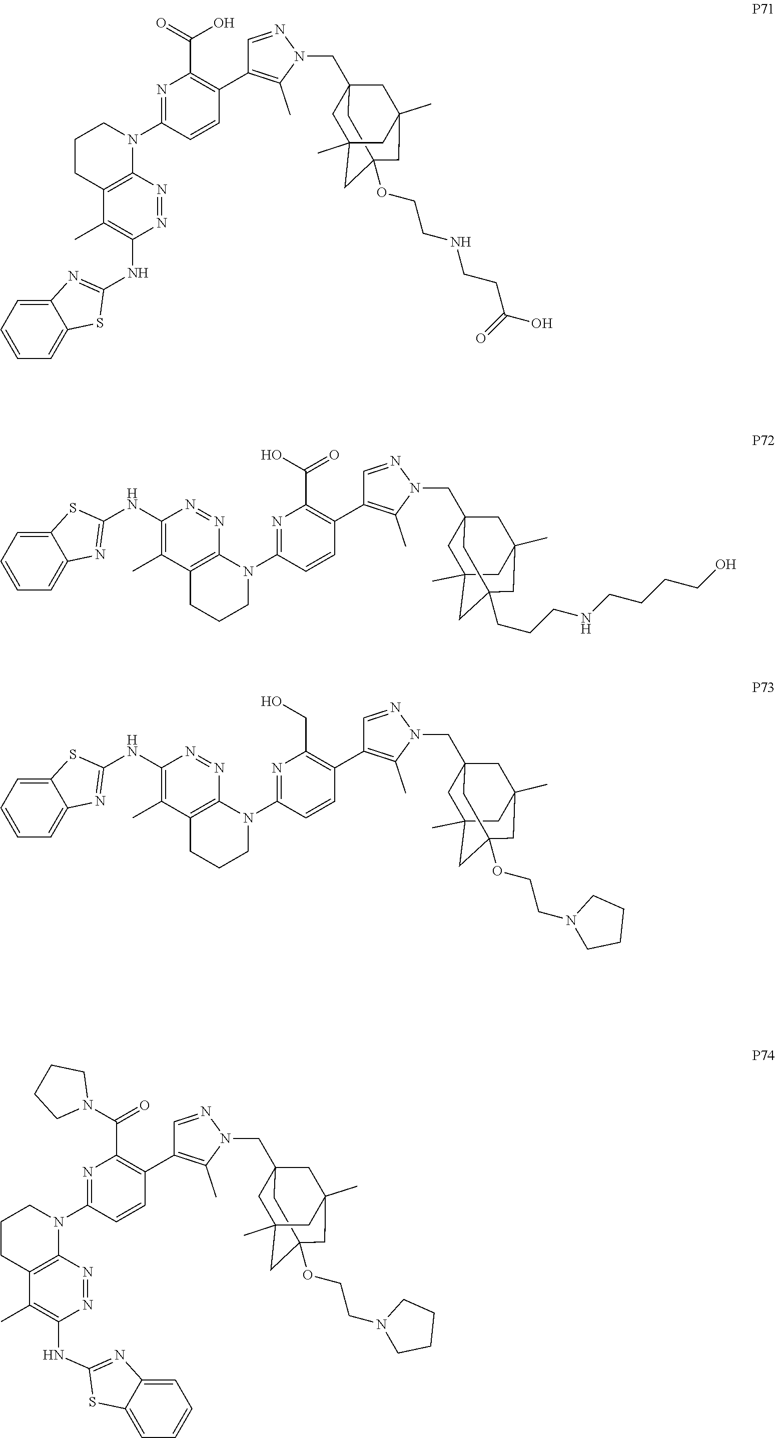 [SMILES: Cc1c(Nc2nc3ccccc3s2)nnc2c1CCCN2c1ccc(-c2cnn(CC34CC5(C)CC(C)(C3)CC(OCCNCCC(=O)O)(C5)C4)c2C)c(C(=O)O)n1 ; Cc1c(Nc2nc3ccccc3s2)nnc2c1CCCN2c1ccc(-c2cnn(CC34CC5(C)CC(C)(CC(CCCNCCCCO)(C5)C3)C4)c2C)c(C(=O)O)n1 ; Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(CO)n1 ; Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)N2CCCC2)n1] 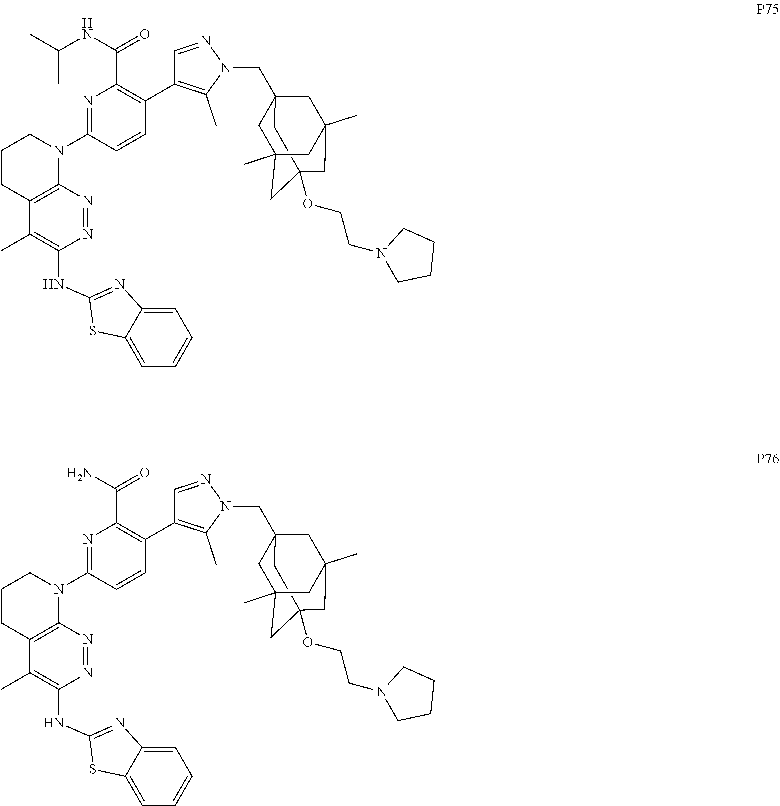 [SMILES: Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(=O)NC(C)C)n1 ; Cc1c(Nc2nc3ccccc3s2)nnc2c1CCCN2c1ccc(-c2cnn(CC34CC5(C)CC(C)(C3)CC(OCCN3CCCC3)(C5)C4)c2C)c(C(N)=O)n1]
or an enantiomer, a diastereoisomer, and/or a pharmaceutically acceptable salt thereof;
(2) D comprises a group represented by a formula selected from 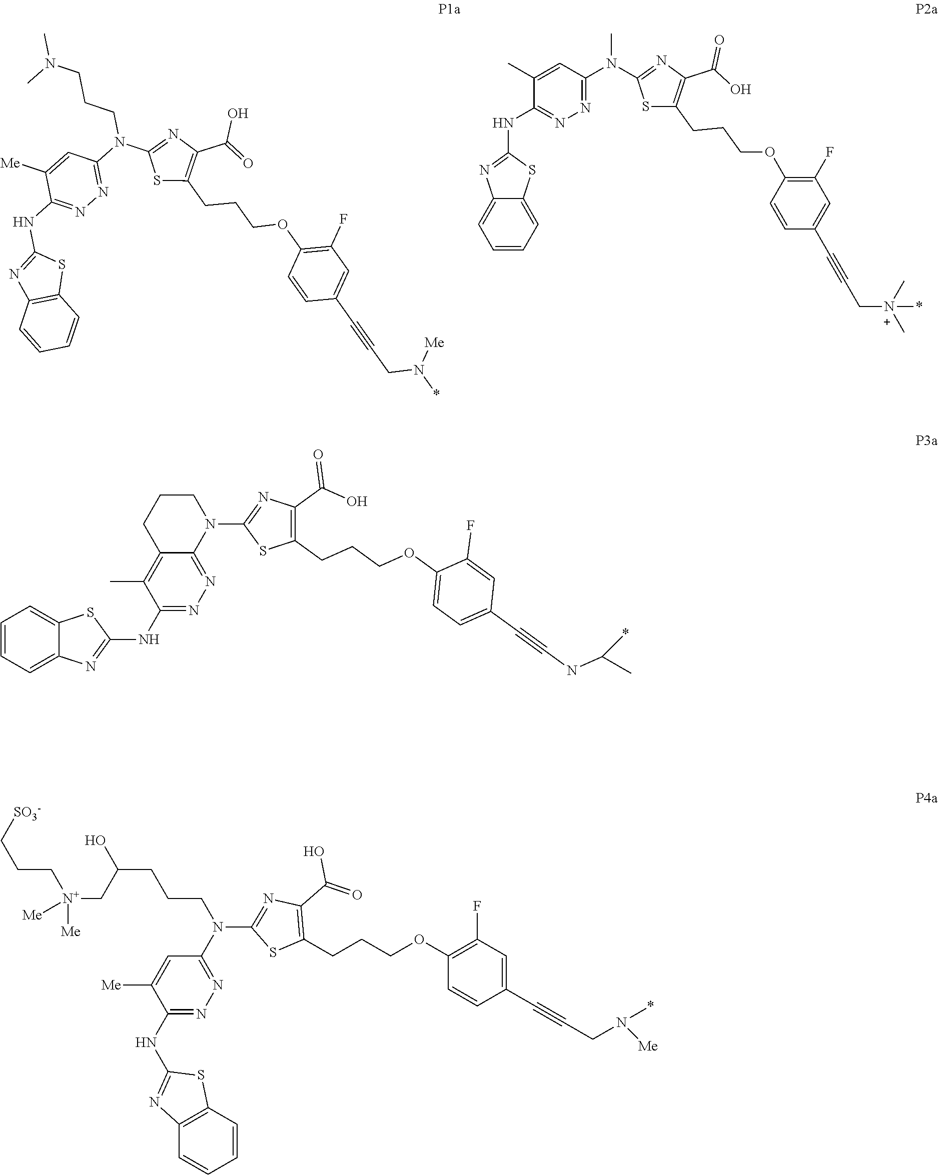 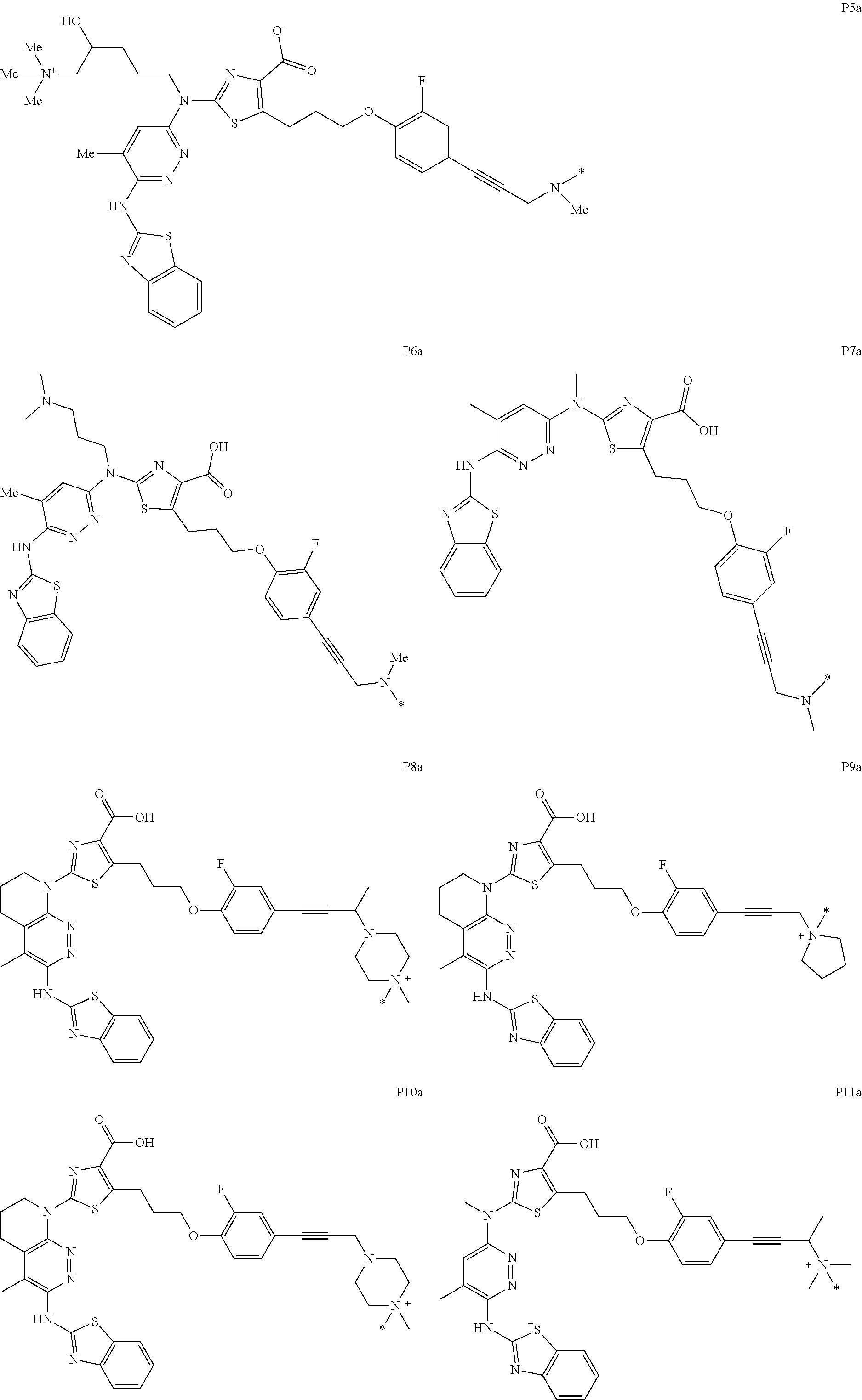 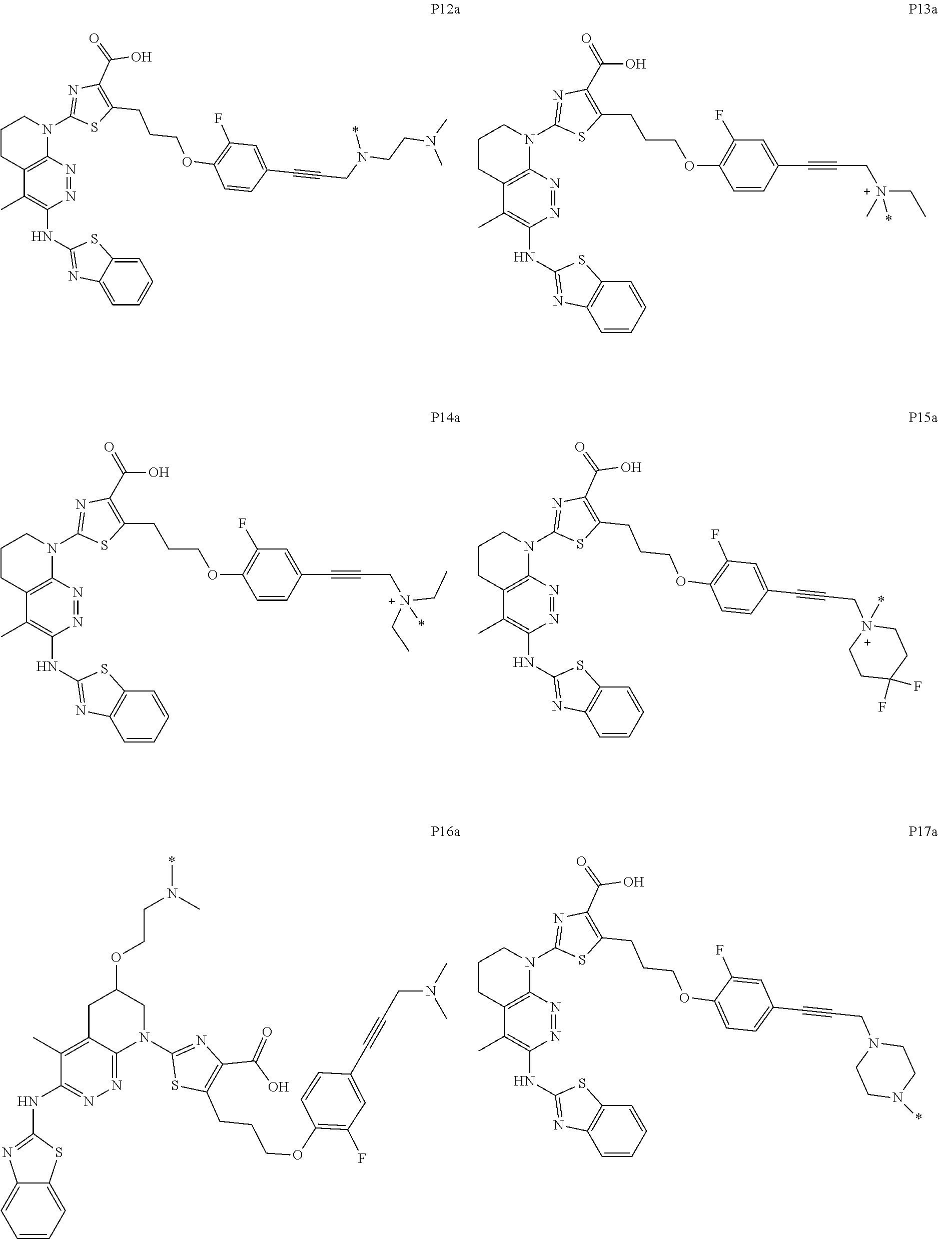 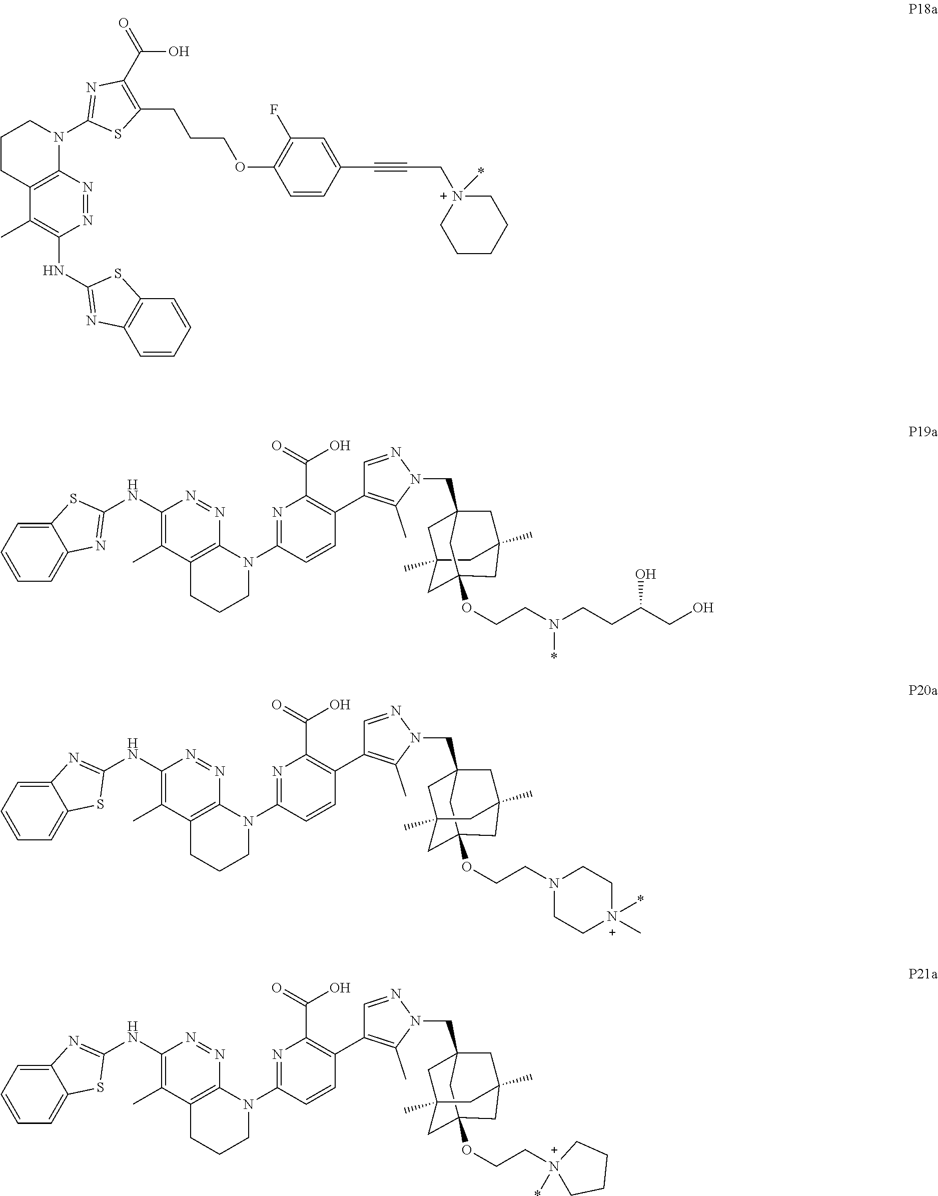 [SMILES: *[N+]1(CC#Cc2ccc(OCCCc3sc(N4CCCc5c4nnc(Nc4nc6ccccc6s4)c5C)nc3C(=O)O)c(F)c2)CCCCC1 ; *N(CCO[C@]12C[C@@]3(C)C[C@](C)(C[C@](Cn4ncc(-c5ccc(N6CCCc7c6nnc(Nc6nc8ccccc8s6)c7C)nc5C(=O)O)c4C)(C3)C1)C2)CC[C@H](O)CO ; *[N+]1(C)CCN(CCO[C@]23C[C@@]4(C)C[C@](C)(C[C@](Cn5ncc(-c6ccc(N7CCCc8c7nnc(Nc7nc9ccccc9s7)c8C)nc6C(=O)O)c5C)(C4)C2)C3)CC1 ; *[N+]1(CCO[C@]23C[C@@]4(C)C[C@](C)(C[C@](Cn5ncc(-c6ccc(N7CCCc8c7nnc(Nc7nc9ccccc9s7)c8C)nc6C(=O)O)c5C)(C4)C2)C3)CCCC1] 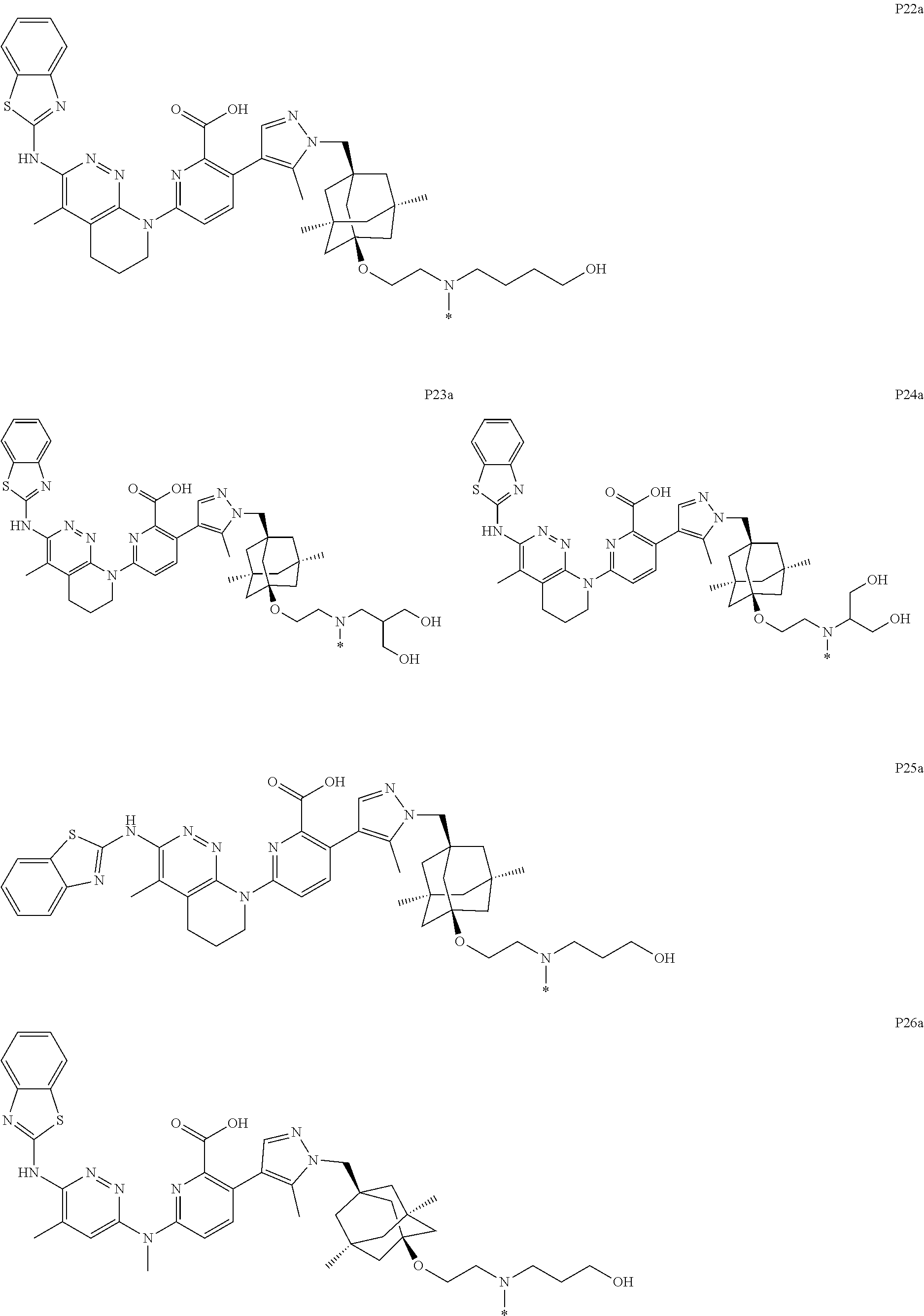 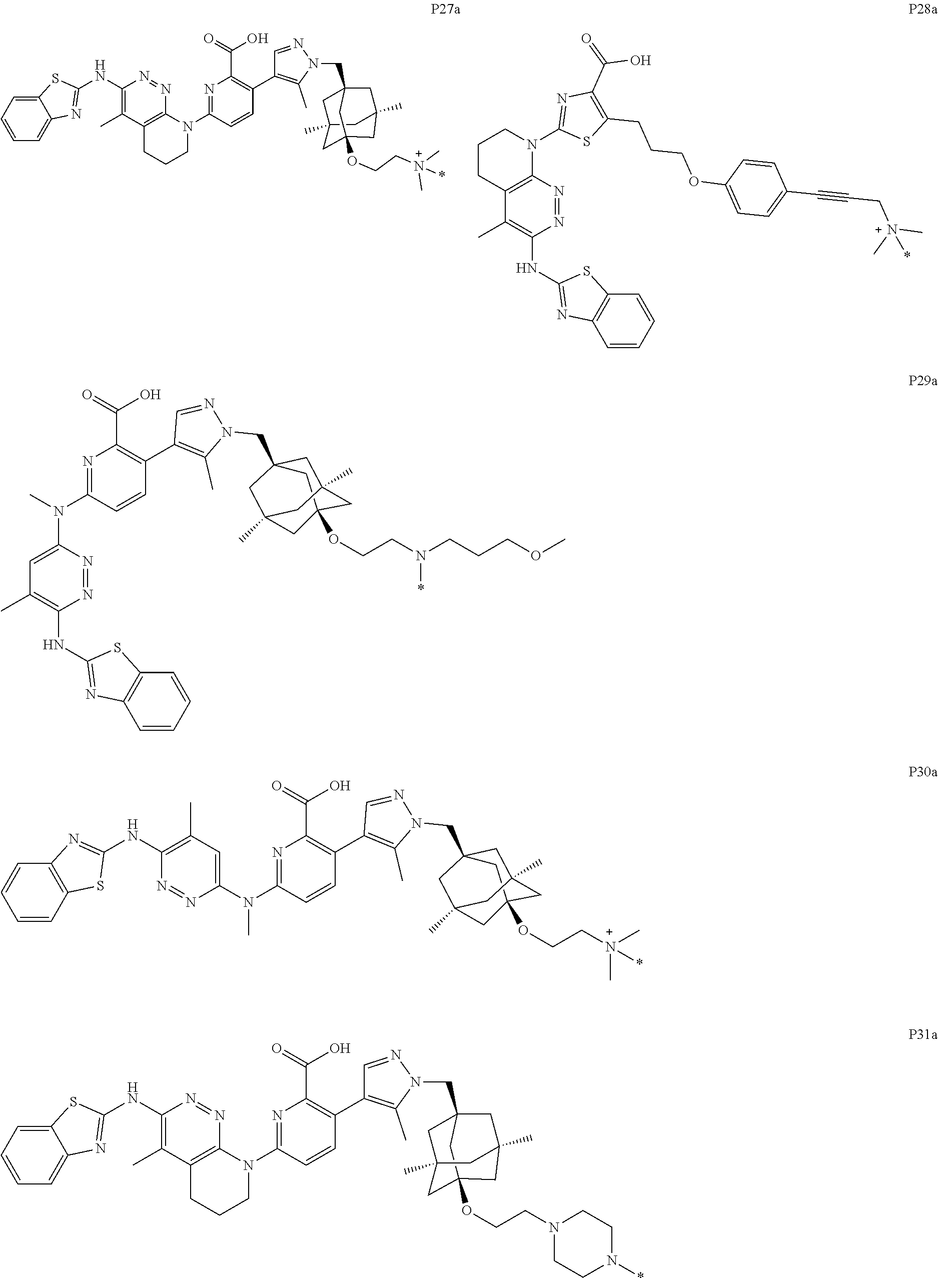 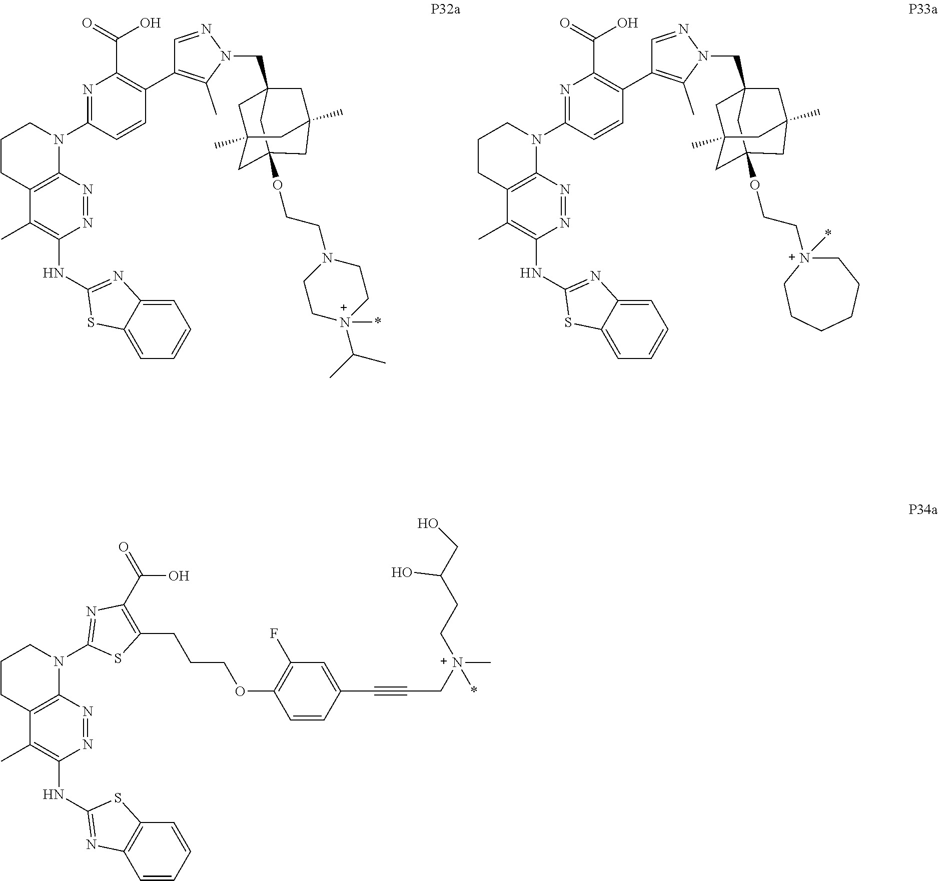 [SMILES: *[N+]1(C(C)C)CCN(CCO[C@]23C[C@@]4(C)C[C@](C)(C[C@](Cn5ncc(-c6ccc(N7CCCc8c7nnc(Nc7nc9ccccc9s7)c8C)nc6C(=O)O)c5C)(C4)C2)C3)CC1 ; *[N+]1(CCO[C@]23C[C@@]4(C)C[C@](C)(C[C@](Cn5ncc(-c6ccc(N7CCCc8c7nnc(Nc7nc9ccccc9s7)c8C)nc6C(=O)O)c5C)(C4)C2)C3)CCCCCC1 ; *[N+](C)(CC#Cc1ccc(OCCCc2sc(N3CCCc4c3nnc(Nc3nc5ccccc5s3)c4C)nc2C(=O)O)c(F)c1)CCC(O)CO] 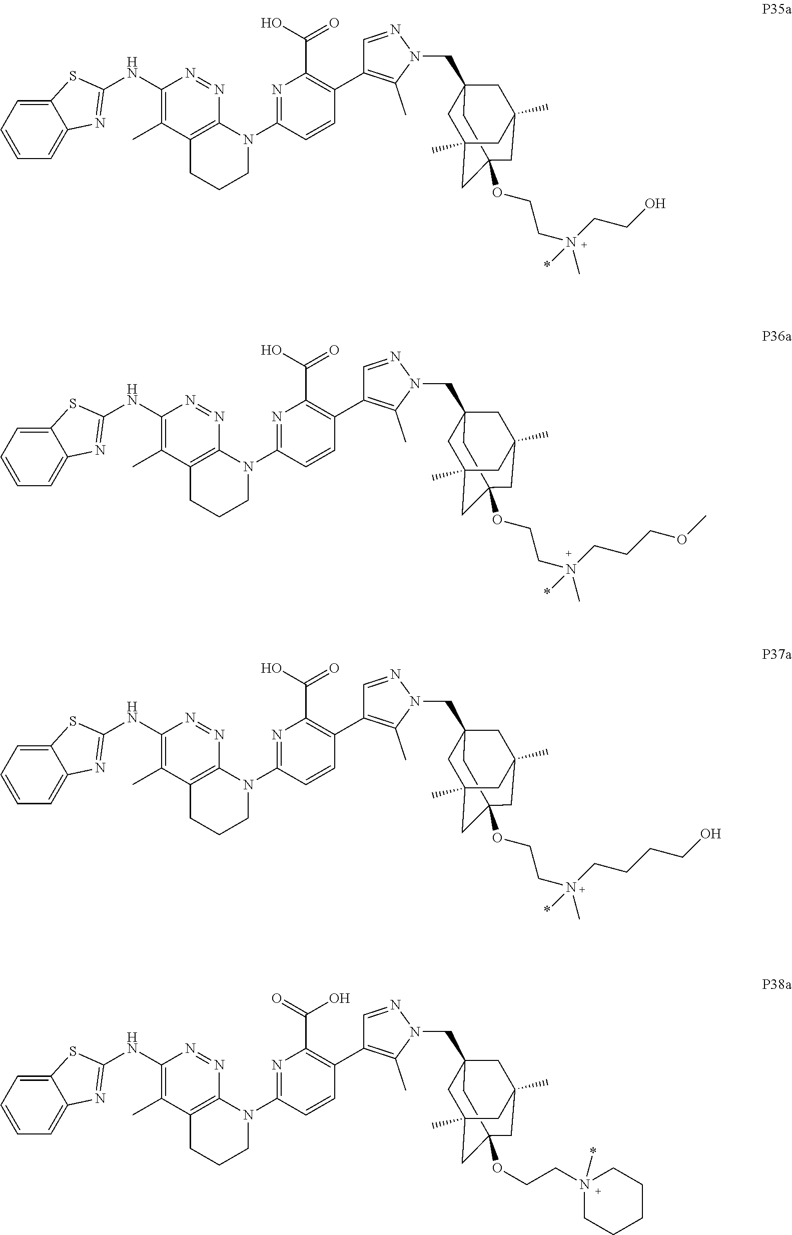 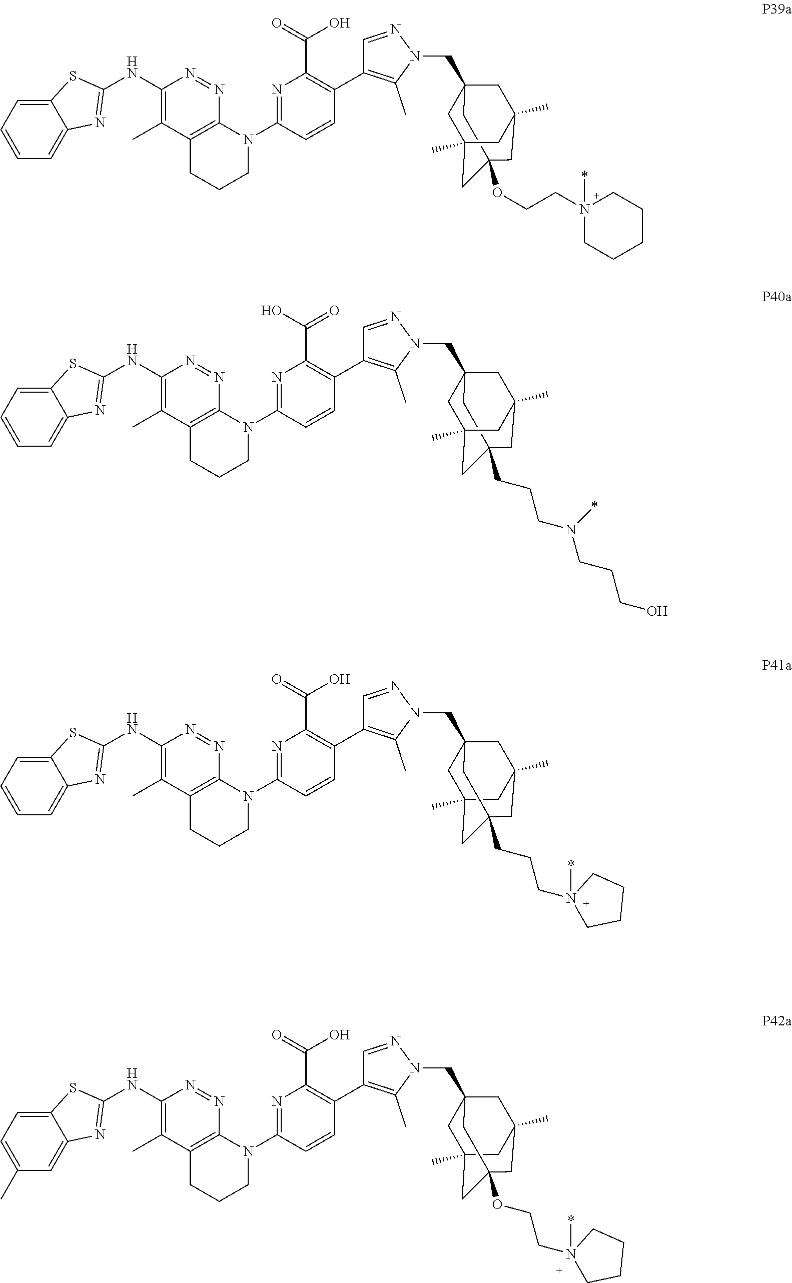 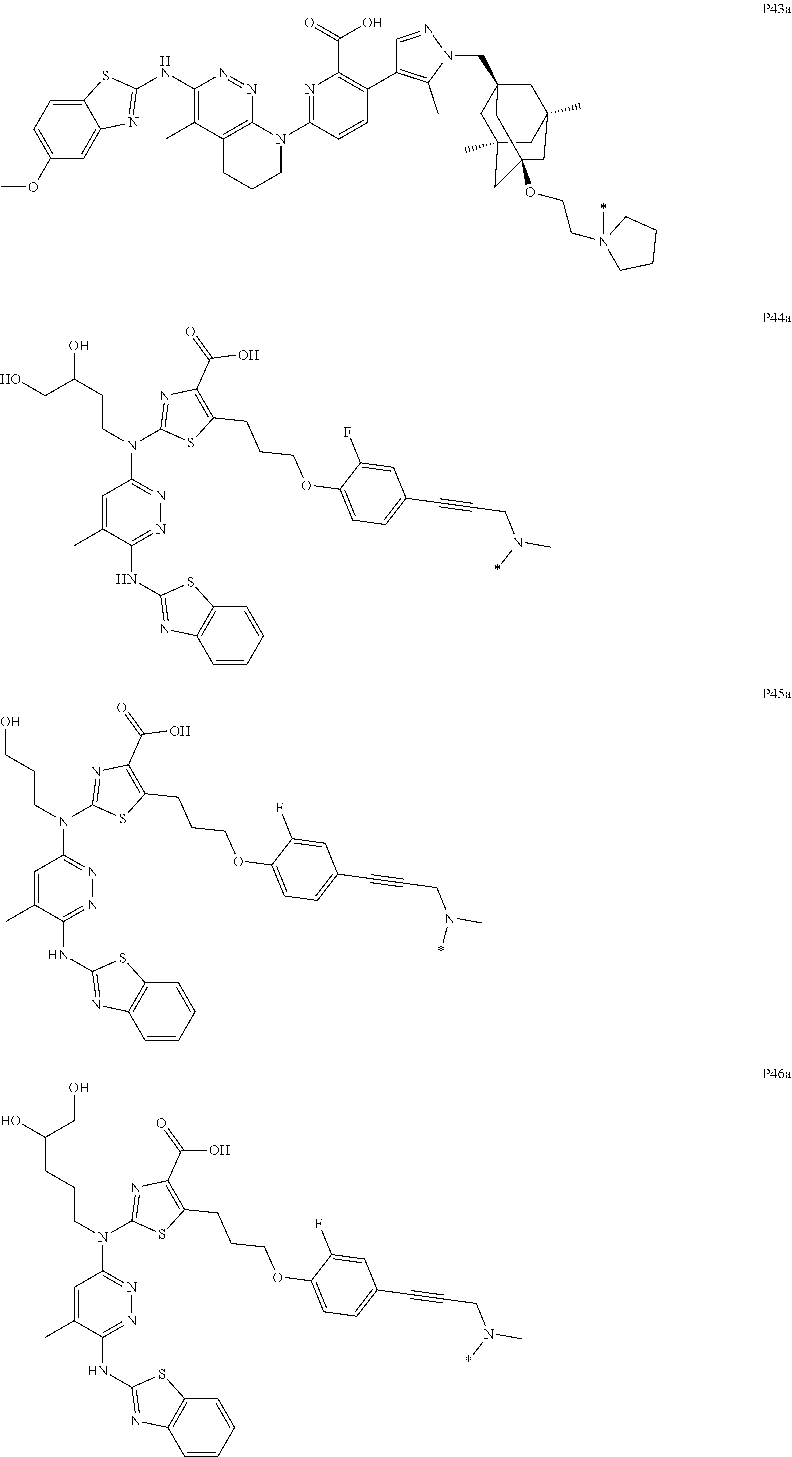 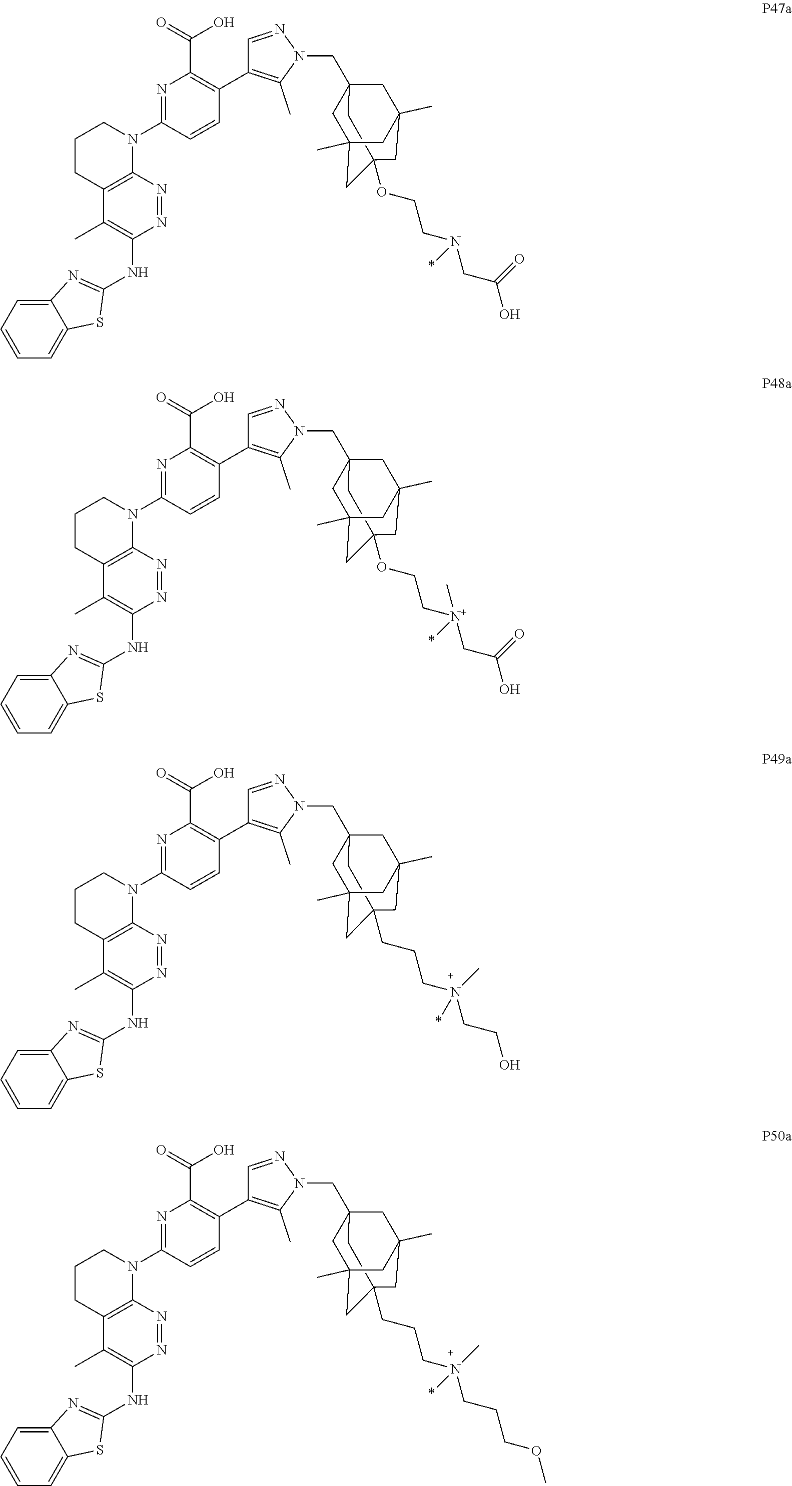 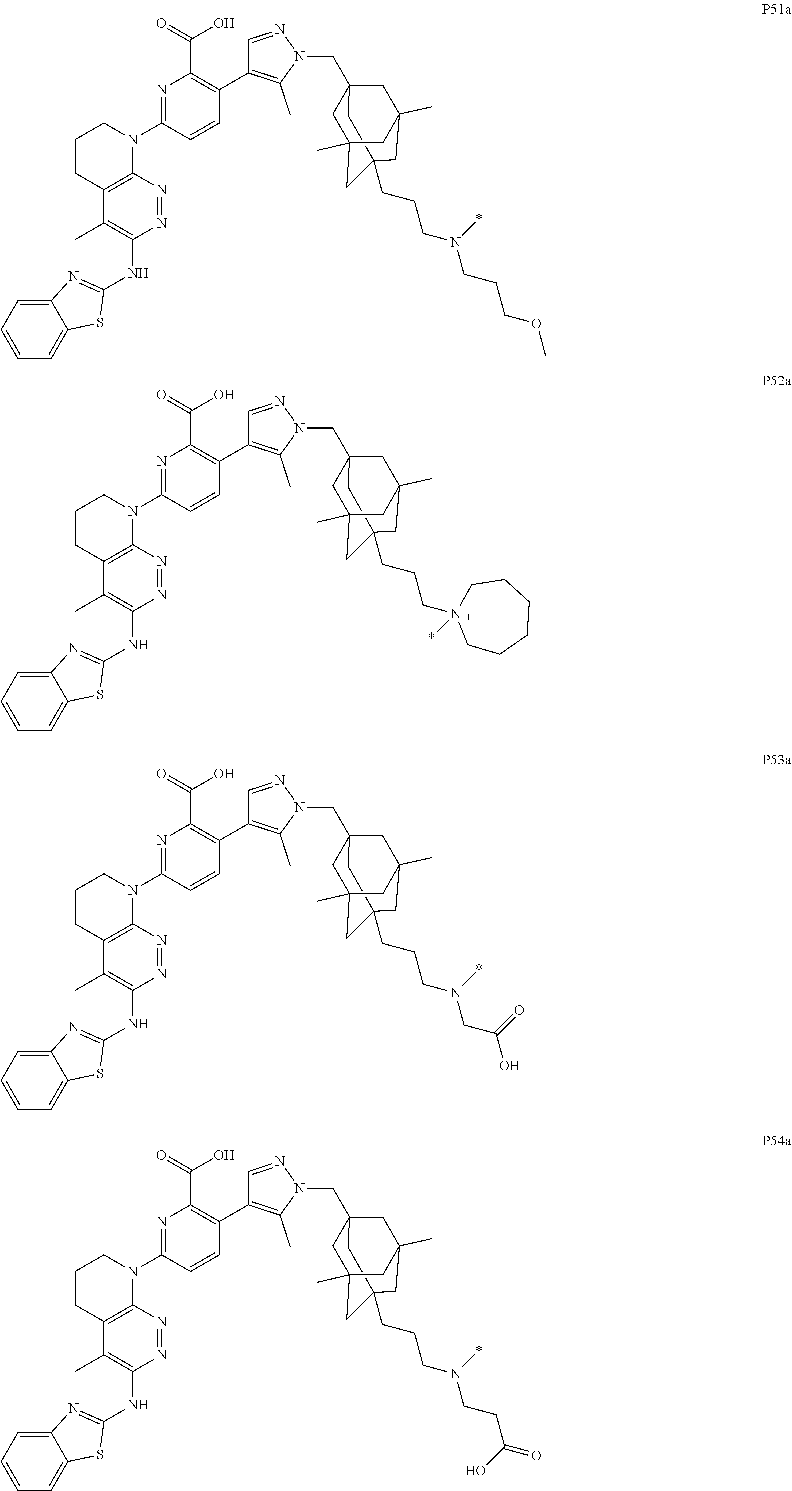 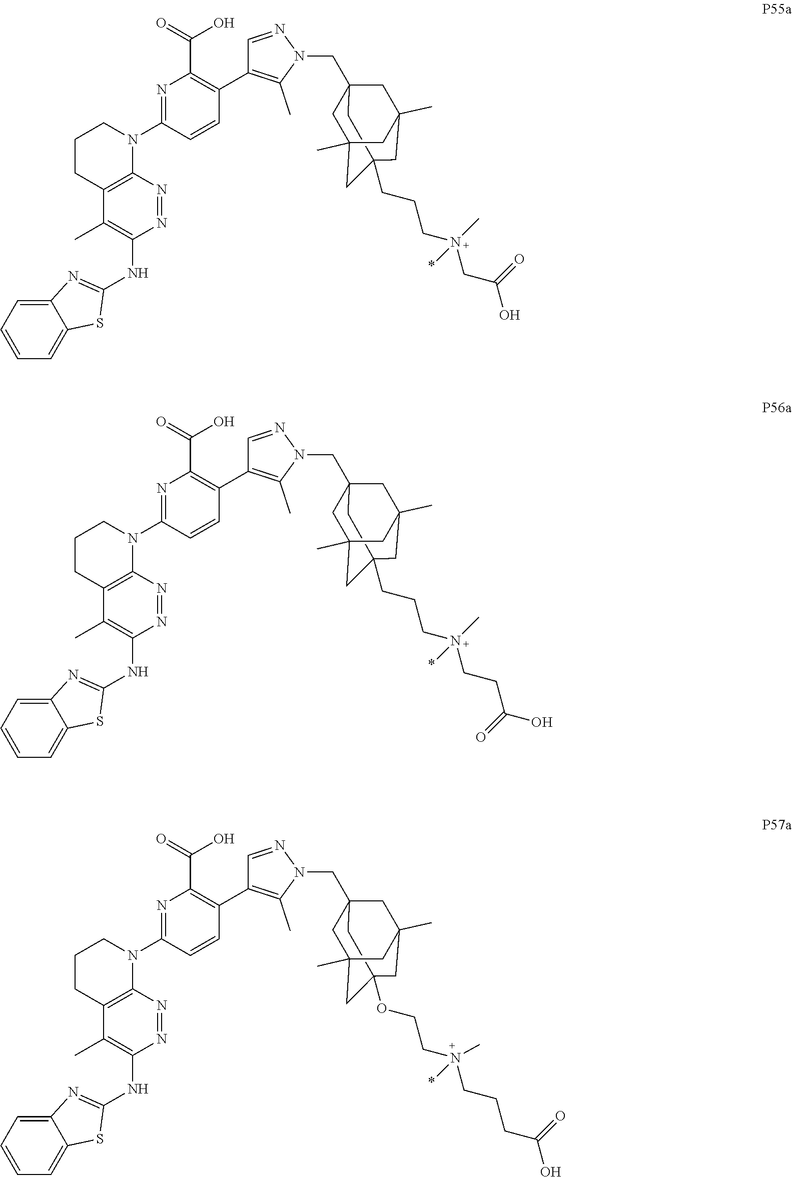 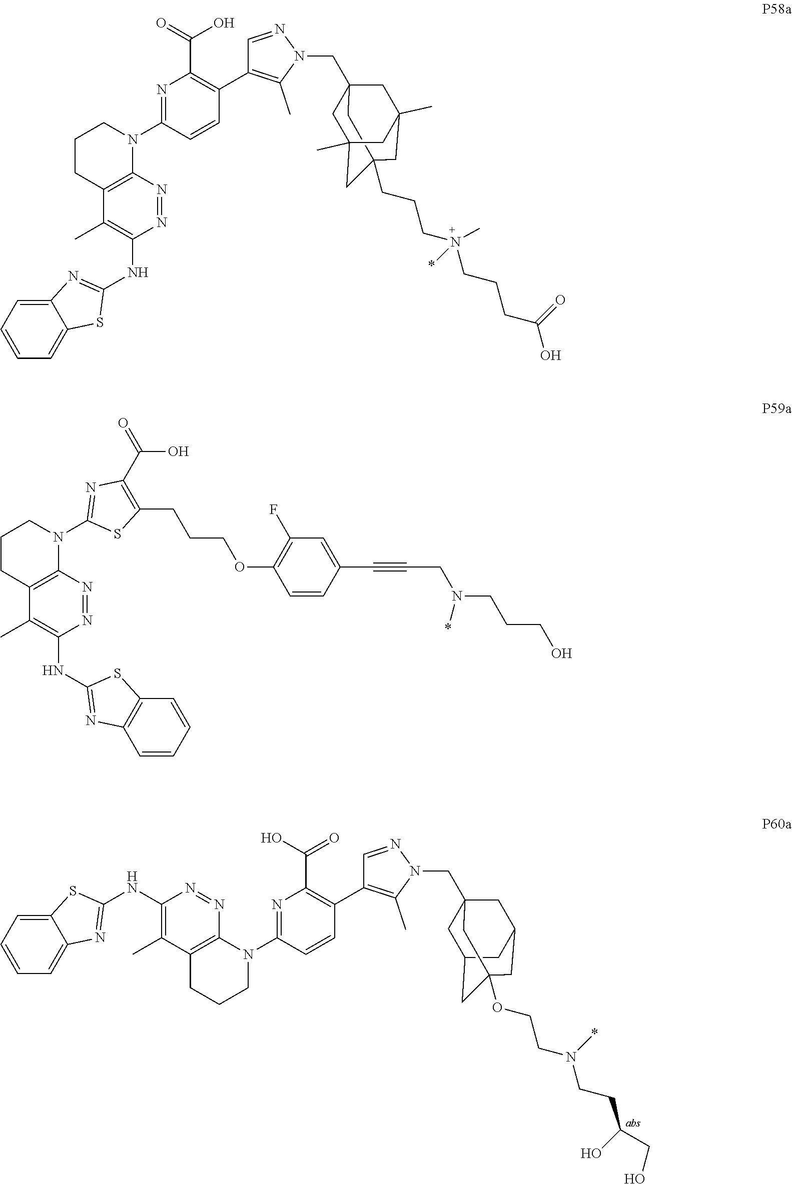 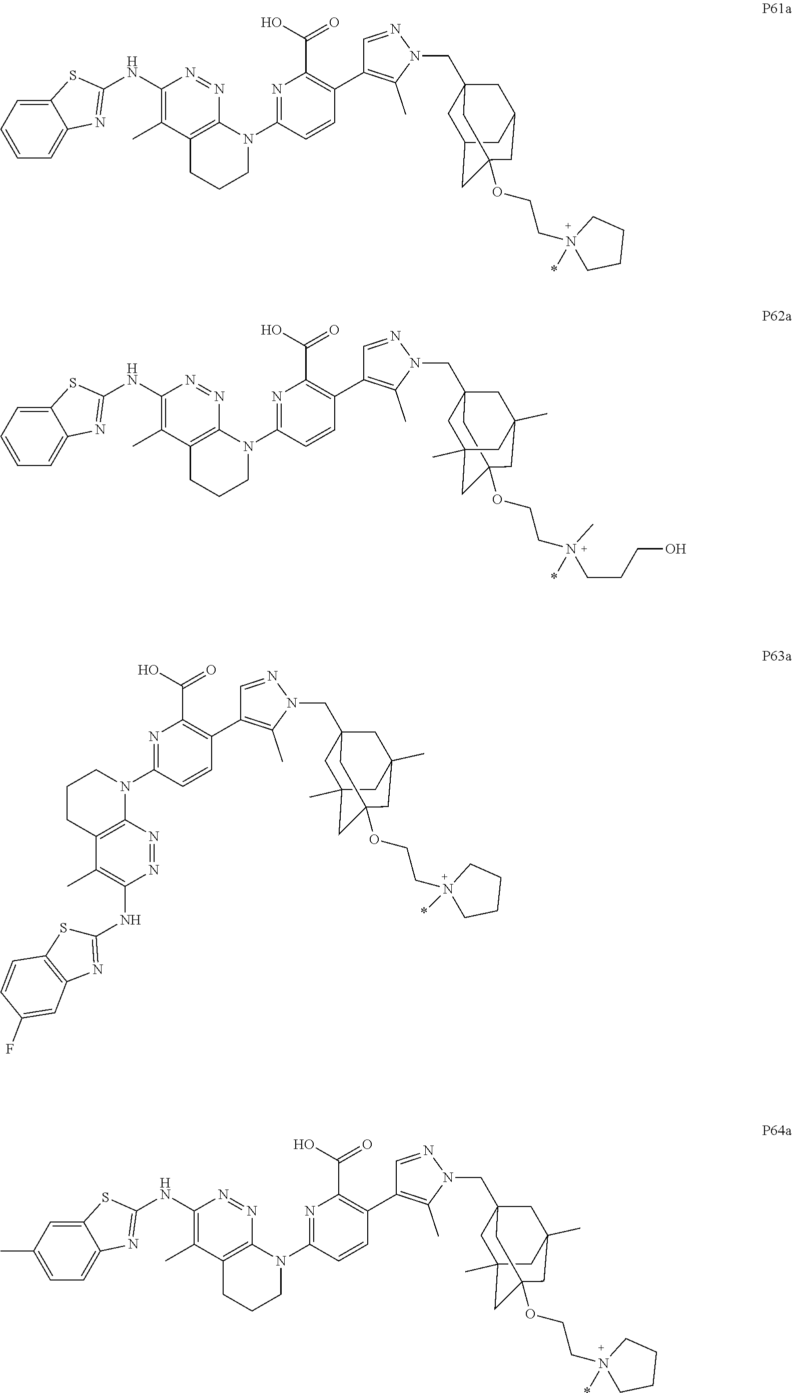 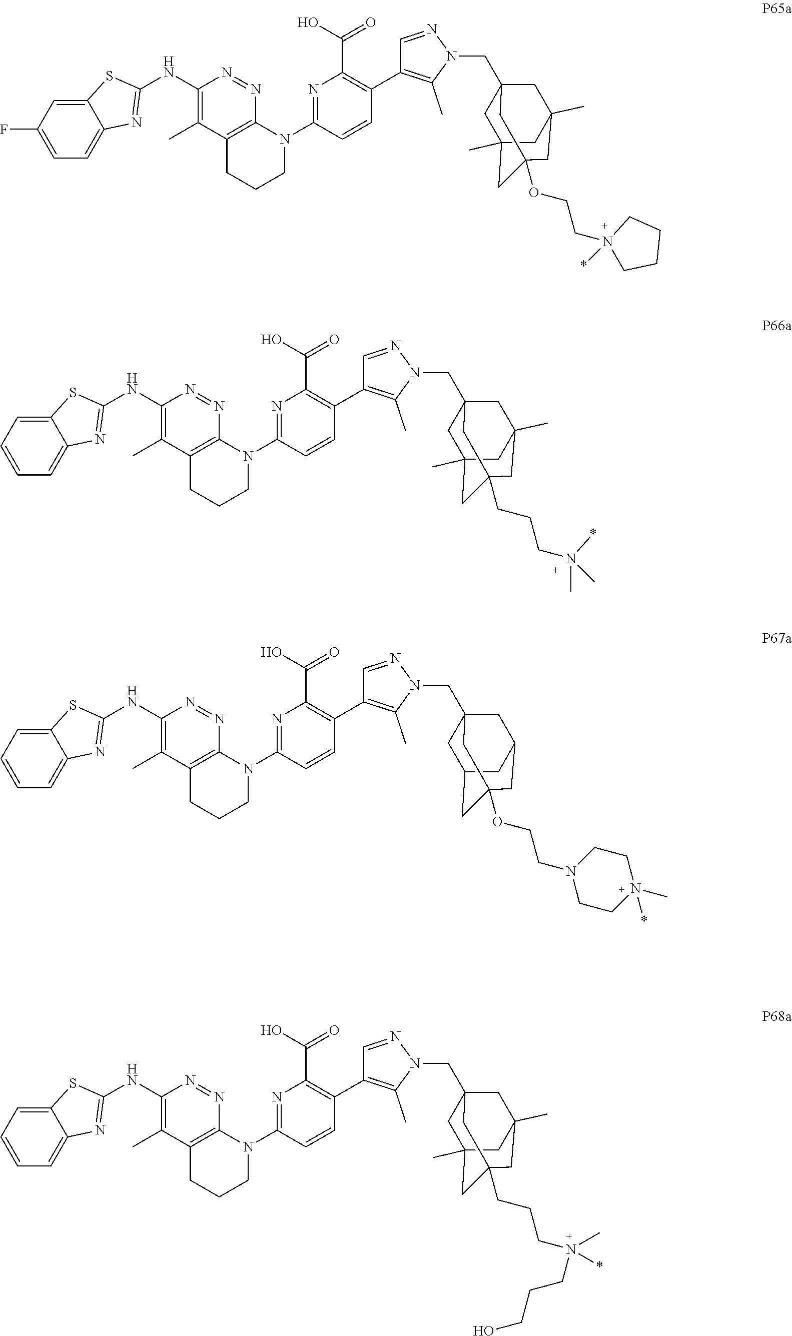 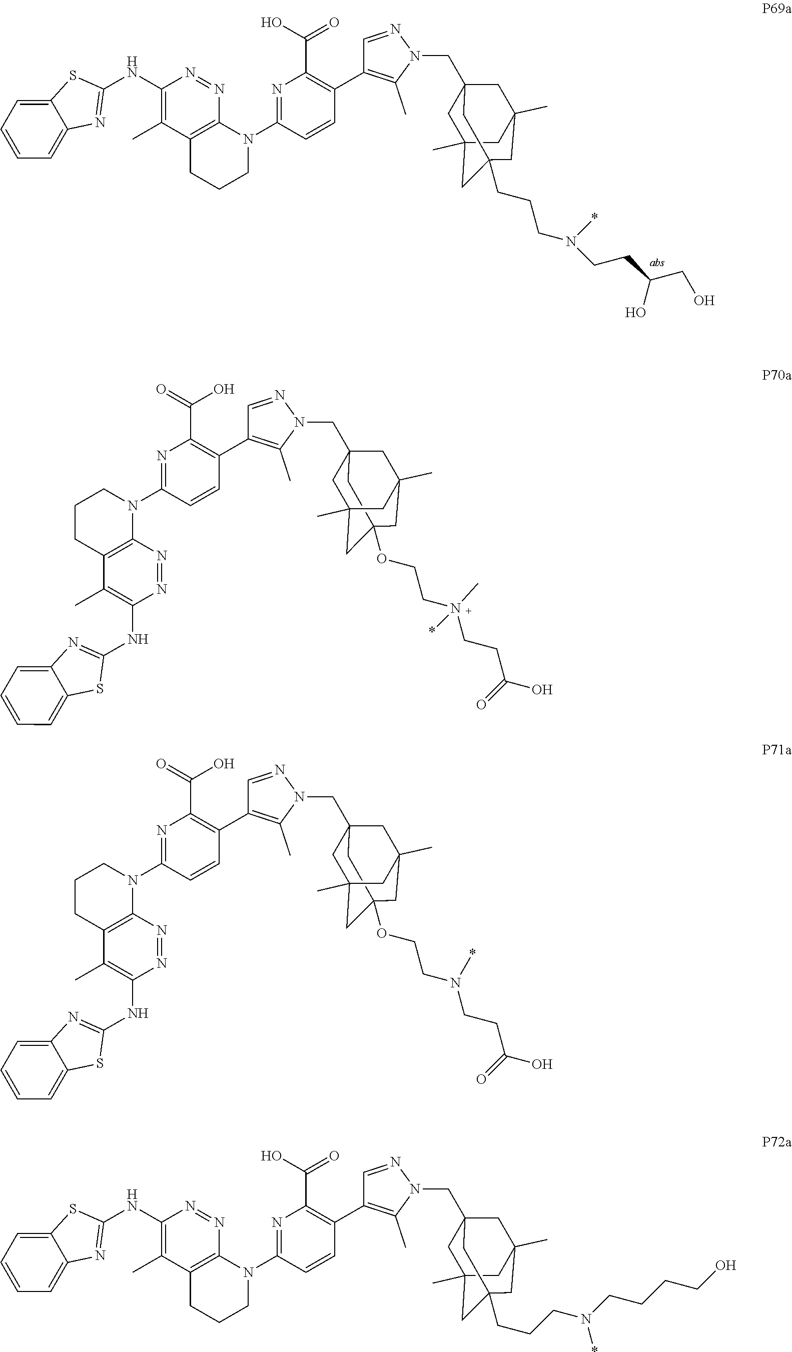 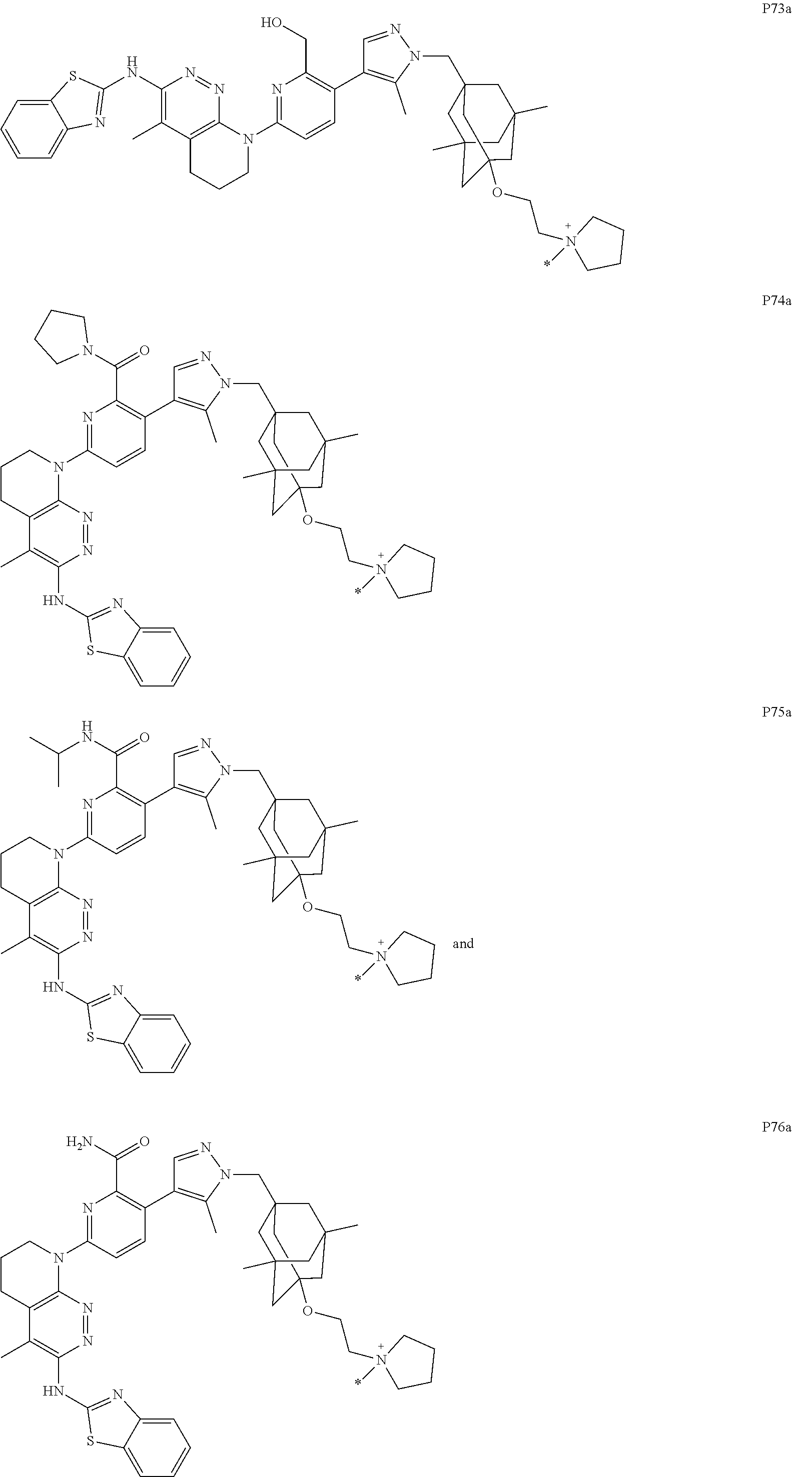
or an enantiomer, a diastereoisomer, and/or pharmaceutically acceptable salt thereof.

56. (canceled)

57. The antibody-drug conjugate of claim 1, wherein-(L-D) is formed from a compound selected from;

| Name | Linker Payload Structure |
|---|---|
| L1A-P1 | 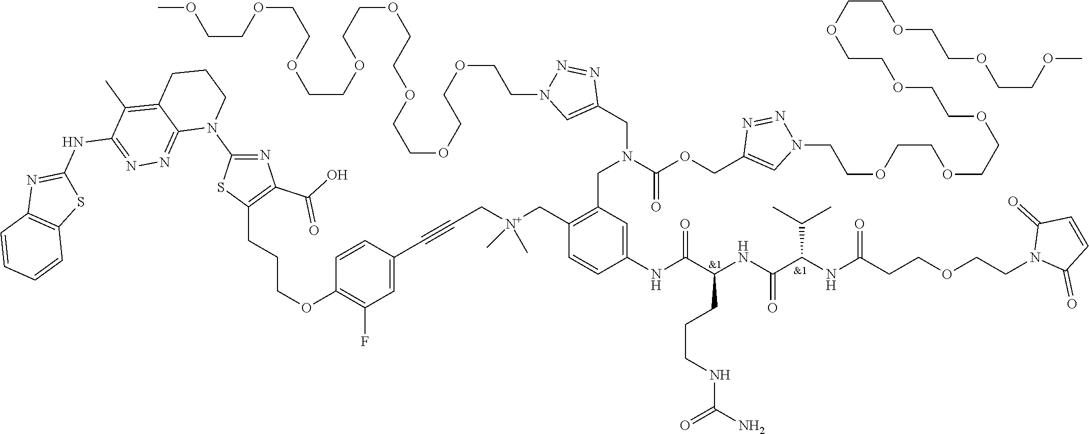 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](NC(=O)CCOCCN3C(=O)C=CC3=O)C(C)C)ccc2C[N+](C)(C)CC#Cc2ccc(OCCCc3sc(N4CCCc5c4nnc(Nc4nc6ccccc6s4)c5C)nc3C(=O)O)c(F)c2)nn1] |
| L1A-P2 | 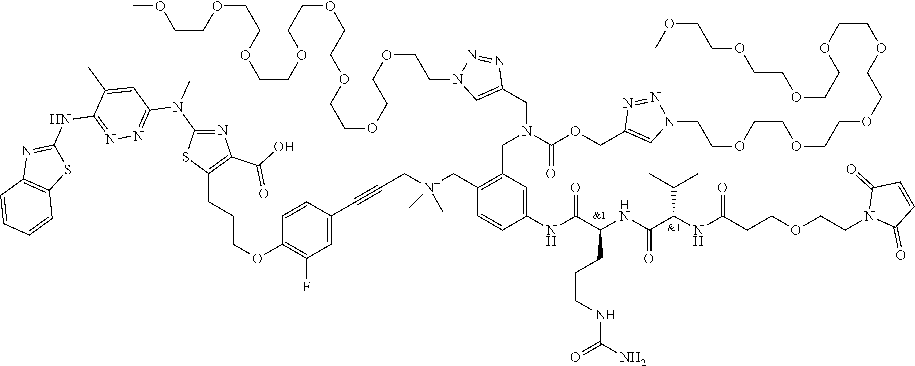 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](NC(=O)CCOCCN3C(=O)C=CC3=O)C(C)C)ccc2C[N+](C)(C)CC#Cc2ccc(OCCCc3sc(N(C)c4cc(C)c(Nc5nc6ccccc6s5)nn4)nc3C(=O)O)c(F)c2)nn1] |
| L1C-P3 | 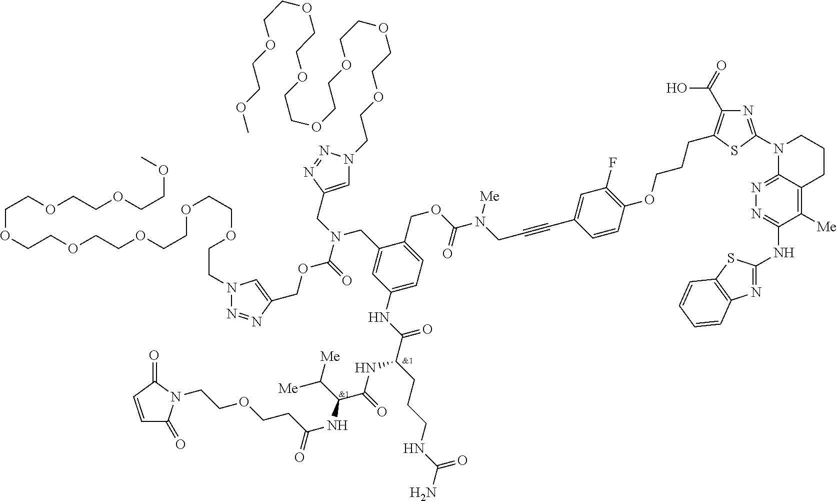 [SMILES: COCCOCCOCCOCCOCCOCCOCCOCCn1cc(COC(=O)N(Cc2cn(CCOCCOCCOCCOCCOCCOCCOCCOC)nn2)Cc2cc(NC(=O)[C@H](CCCNC(N)=O)NC(=O)[C@@H](NC(=O)CCOCCN3C(=O)C=CC3=O)C(C)C)ccc2COC(=O)N(C)CC#Cc2ccc(OCCCc3sc(N4CCCc5c4nnc(Nc4nc6ccccc6s4)c5C)nc3C(=O)O)c(F)c2)nn1] |
| L3A-P1 | 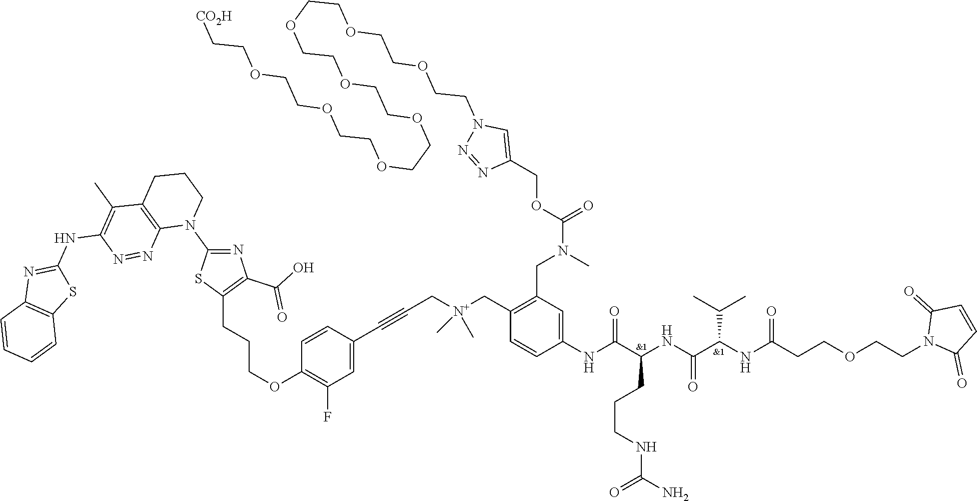 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)OCc3cn(CCOCCOCCOCCOCCOCCOCCOCCOCCC(=O)O)nn3)cc2F)s1] |
| L3C-P4 | 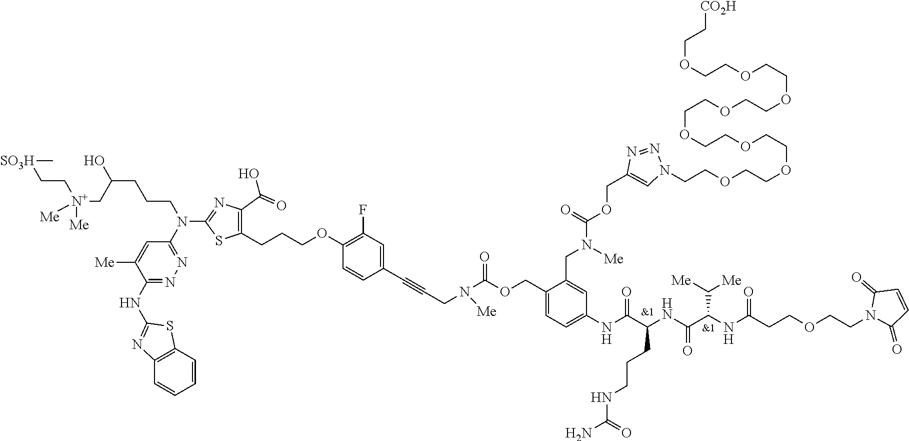 |
| L3C-P5 | 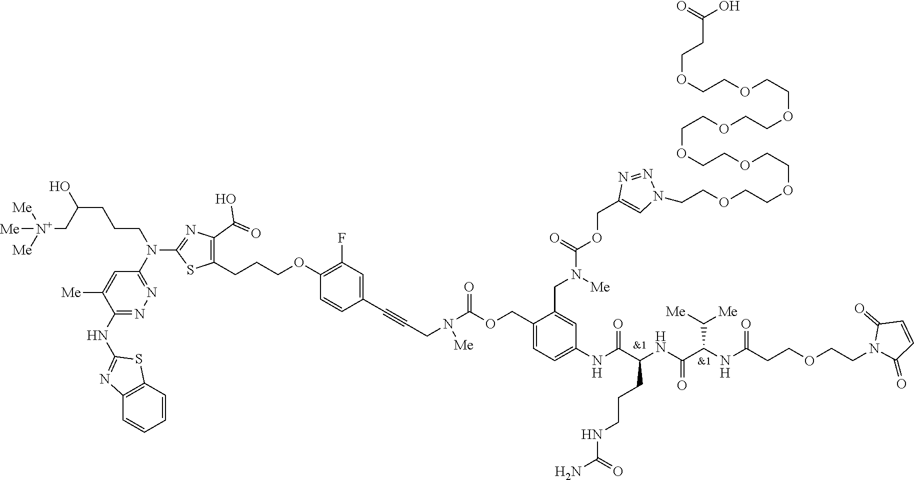 [SMILES: Cc1cc(N(CCCC(O)C[N+](C)(C)C)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4CN(C)C(=O)OCc4cn(CCOCCOCCOCCOCCOCCOCCOCCOCCC(=O)O)nn4)cc3F)s2)nnc1Nc1nc2ccccc2s1] |
| L3C-P3 | 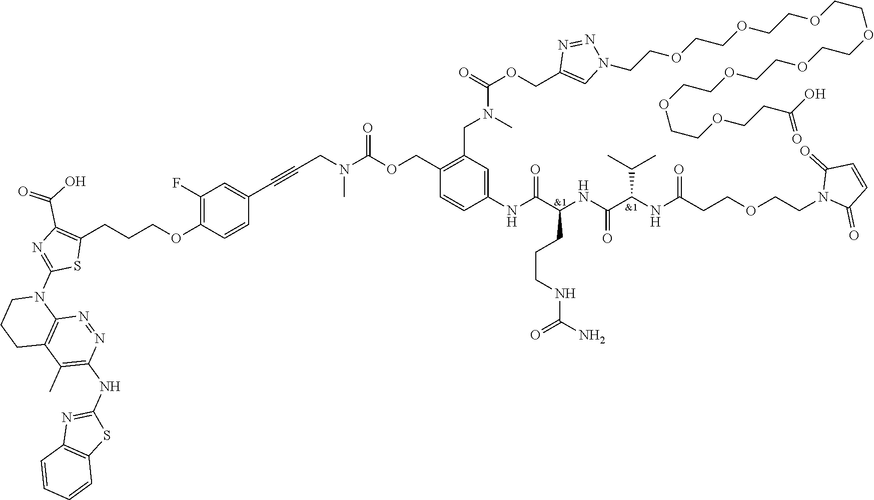 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3CN(C)C(=O)OCc3cn(CCOCCOCCOCCOCCOCCOCCOCCOCCC(=O)O)nn3)cc2F)s1] |
| L4A-P1 | 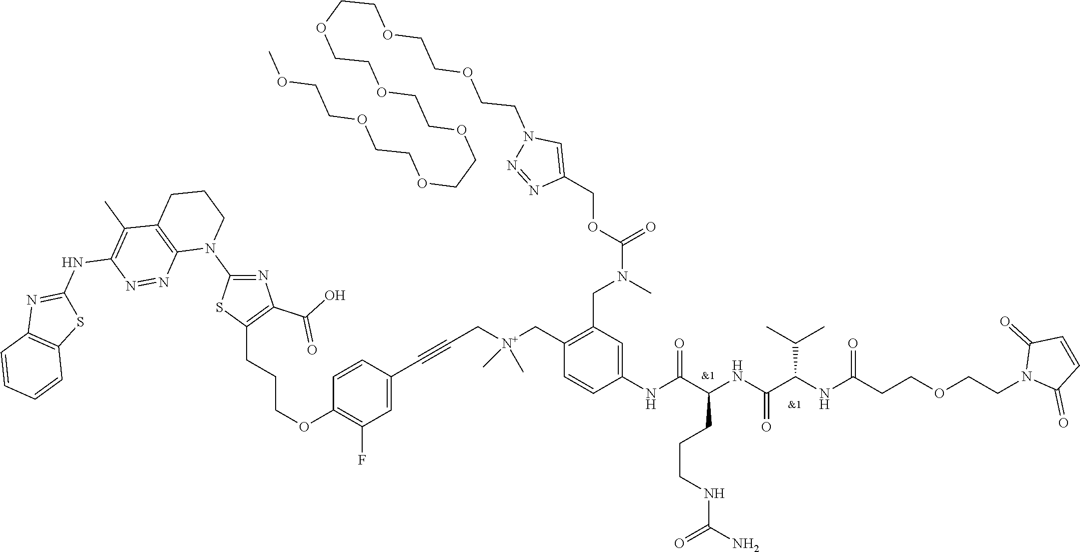 [SMILES: COCCOCCOCCOCCOCCOCCOCCOCCn1cc(COC(=O)N(C)Cc2cc(NC(=O)[C@H](CCCNC(N)=O)NC(=O)[C@@H](NC(=O)CCOCCN3C(=O)C=CC3=O)C(C)C)ccc2C[N+](C)(C)CC#Cc2ccc(OCCCc3sc(N4CCCc5c4nnc(Nc4nc6ccccc6s4)c5C)nc3C(=O)O)c(F)c2)nn1] |
| L7A-P1 | 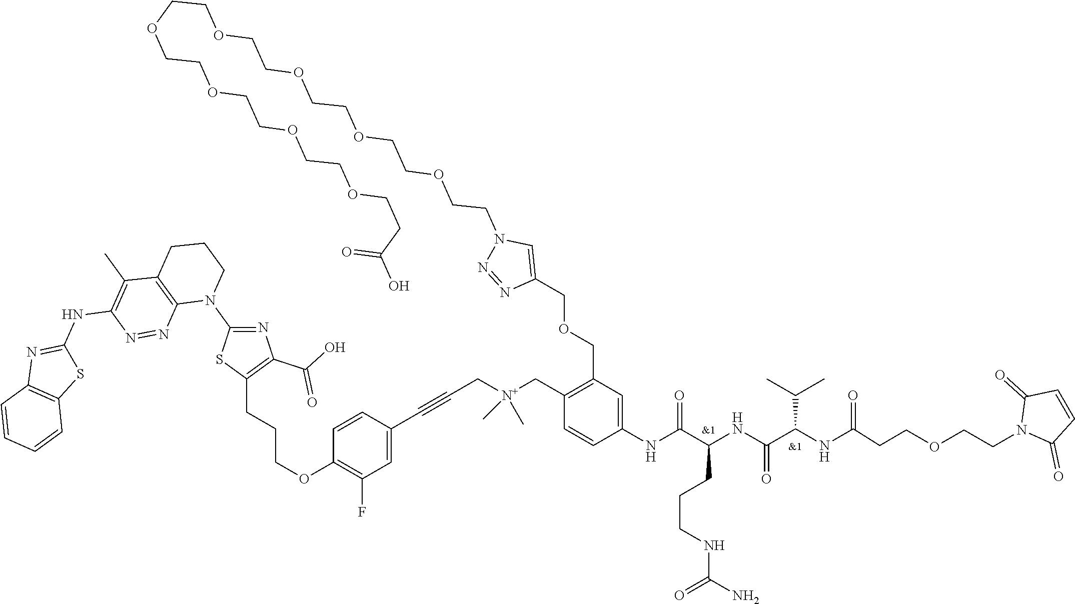 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3COCc3cn(CCOCCOCCOCCOCCOCCOCCOCCOCCC(=O)O)nn3)cc2F)s1] |
| L7A-P2 | 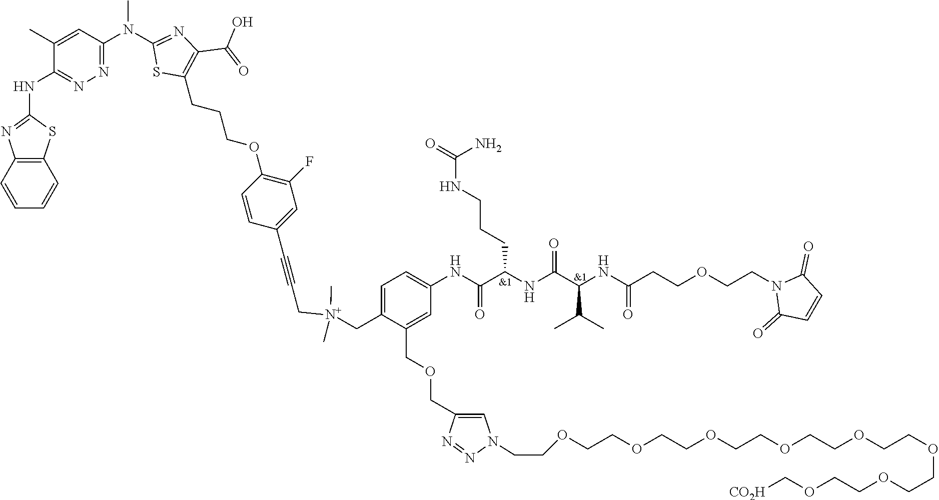 [SMILES: Cc1cc(N(C)c2nc(C(=O)O)c(CCCOc3ccc(C#CC[N+](C)(C)Cc4ccc(NC(=O)[C@H](CCCNC(N)=O)NC(=O)[C@@H](NC(=O)CCOCCN5C(=O)C=CC5=O)C(C)C)cc4COCc4cn(CCOCCOCCOCCOCCOCCOCCOCCOCC(=O)O)nn4)cc3F)s2)nnc1Nc1nc2ccccc2s1] |
| L7C-P3 | 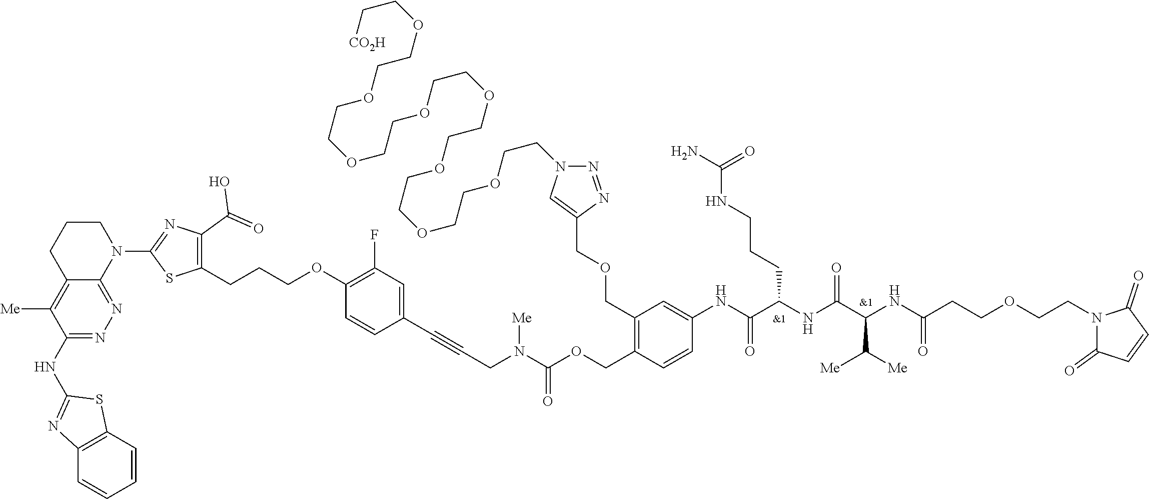 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3COCc3cn(CCOCCOCCOCCOCCOCCOCCOCCOCCC(=O)O)nn3)cc2F)s1] |
| L7C-P6 | 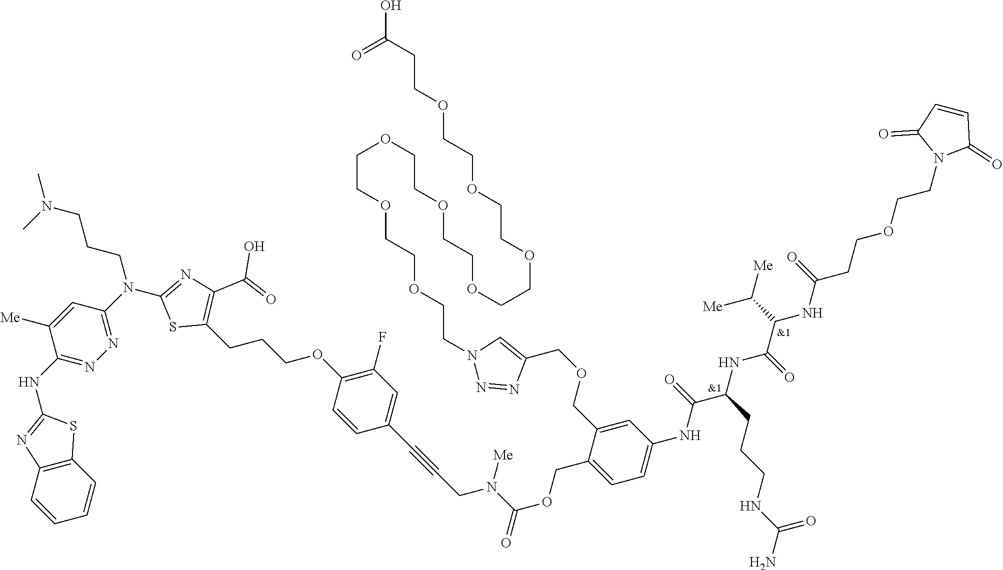 |
| L7C-P7 | 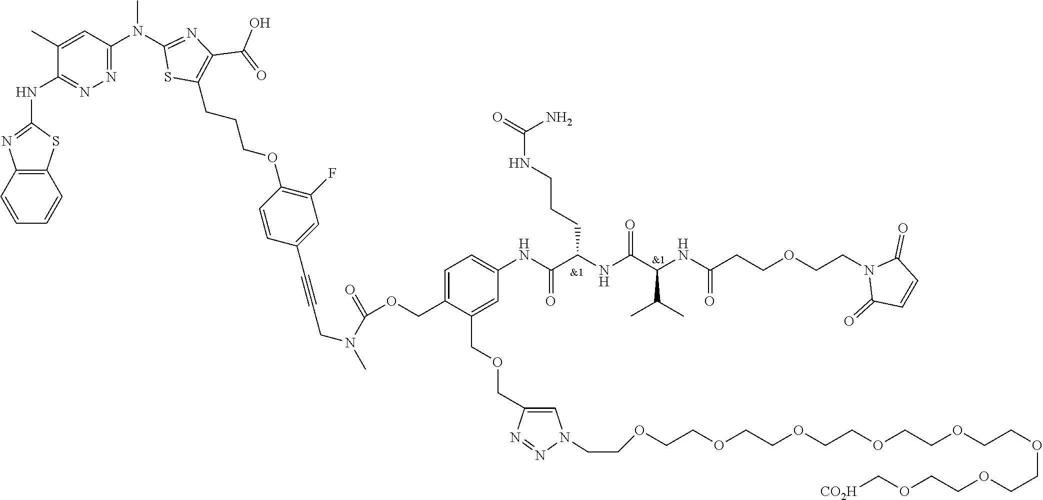 [SMILES: Cc1cc(N(C)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4COCc4cn(CCOCCOCCOCCOCCOCCOCCOCCOCC(=O)O)nn4)cc3F)s2)nnc1Nc1nc2ccccc2s1] |
| L8A-P1 | 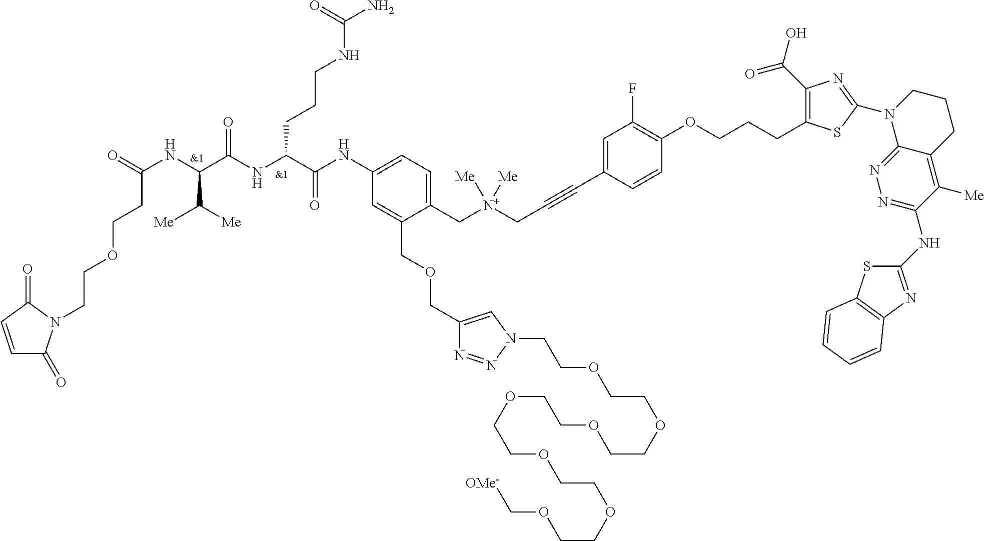 |
| L8C-P7 | 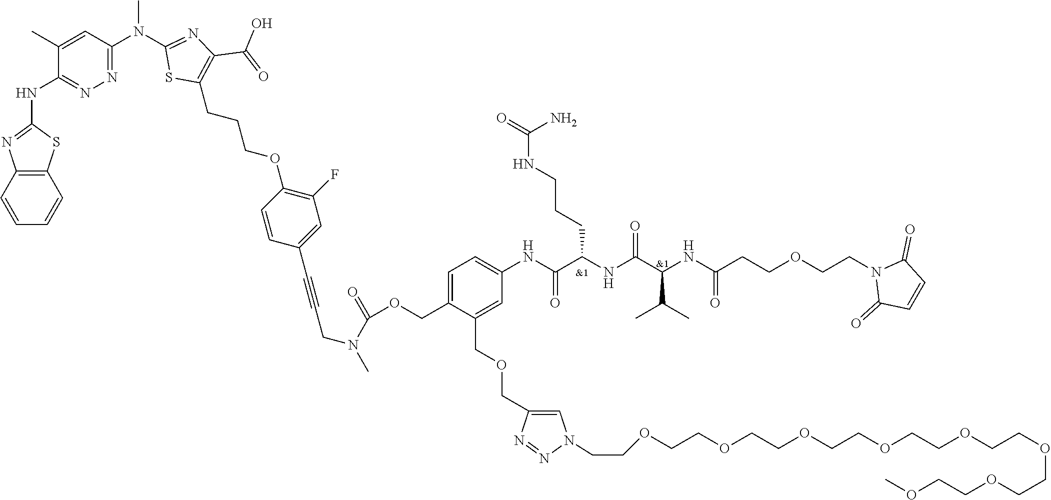 [SMILES: COCCOCCOCCOCCOCCOCCOCCOCCn1cc(COCc2cc(NC(=O)[C@H](CCCNC(N)=O)NC(=O)[C@@H](NC(=O)CCOCCN3C(=O)C=CC3=O)C(C)C)ccc2COC(=O)N(C)CC#Cc2ccc(OCCCc3sc(N(C)c4cc(C)c(Nc5nc6ccccc6s5)nn4)nc3C(=O)O)c(F)c2)nn1] |
| L9A-P8 | 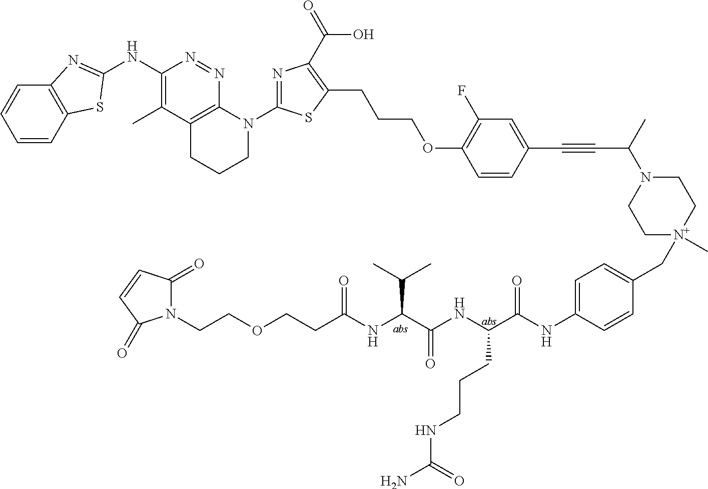 [SMILES: Cc1c(Nc2nc3ccccc3s2)nnc2c1CCCN2c1nc(C(=O)O)c(CCCOc2ccc(C#CC(C)N3CC[N+](C)(Cc4ccc(NC(=O)[C@H](CCCNC(N)=O)NC(=O)[C@@H](NC(=O)CCOCCN5C(=O)C=CC5=O)C(C)C)cc4)CC3)cc2F)s1] |
| L9A-P9 | 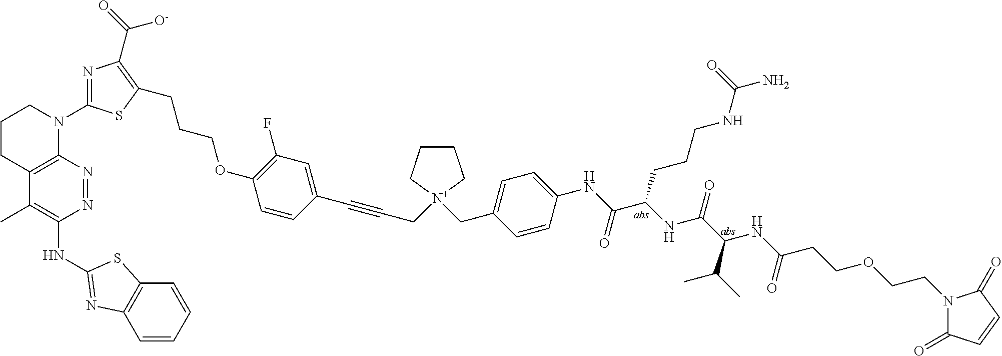 [SMILES: Cc1c(Nc2nc3ccccc3s2)nnc2c1CCCN2c1nc(C(=O)[O-])c(CCCOc2ccc(C#CC[N+]3(Cc4ccc(NC(=O)[C@H](CCCNC(N)=O)NC(=O)[C@@H](NC(=O)CCOCCN5C(=O)C=CC5=O)C(C)C)cc4)CCCC3)cc2F)s1] |
| L9A-P10 | 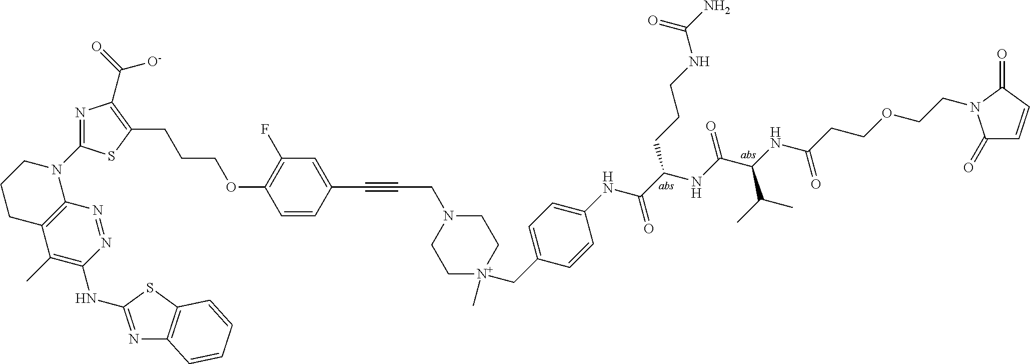 [SMILES: Cc1c(Nc2nc3ccccc3s2)nnc2c1CCCN2c1nc(C(=O)[O-])c(CCCOc2ccc(C#CCN3CC[N+](C)(Cc4ccc(NC(=O)[C@H](CCCNC(N)=O)NC(=O)[C@@H](NC(=O)CCOCCN5C(=O)C=CC5=O)C(C)C)cc4)CC3)cc2F)s1] |
| L9A-P11 | 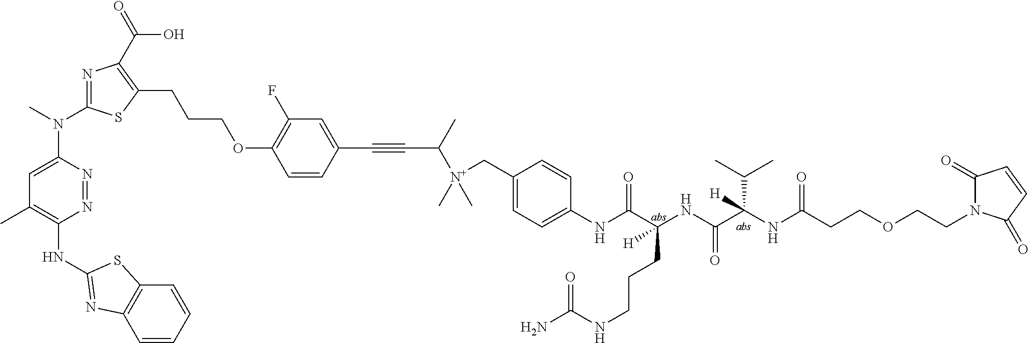 [SMILES: Cc1cc(N(C)c2nc(C(=O)O)c(CCCOc3ccc(C#CC(C)[N+](C)(C)Cc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P12 | 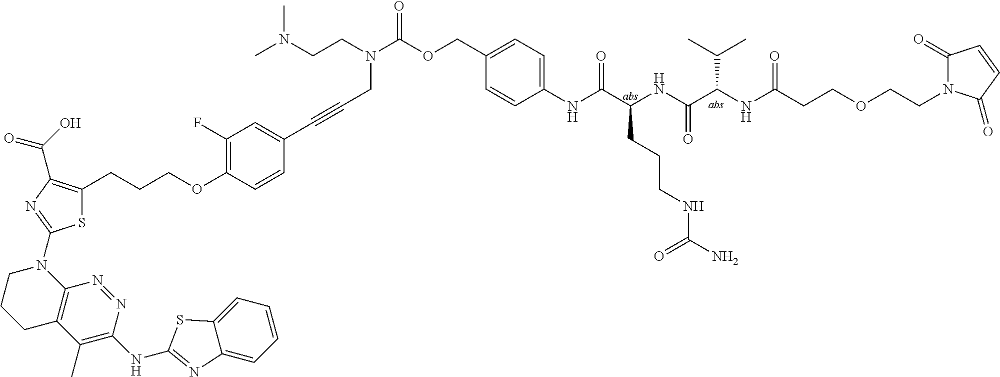 [SMILES: Cc1c(Nc2nc3ccccc3s2)nnc2c1CCCN2c1nc(C(=O)O)c(CCCOc2ccc(C#CCN(CCN(C)C)C(=O)OCc3ccc(NC(=O)[C@H](CCCNC(N)=O)NC(=O)[C@@H](NC(=O)CCOCCN4C(=O)C=CC4=O)C(C)C)cc3)cc2F)s1] |
| L9A-P13 | 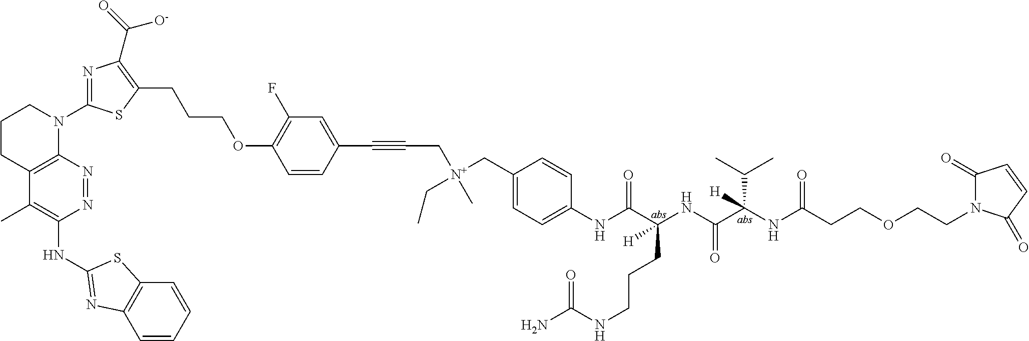 [SMILES: CC[N+](C)(CC#Cc1ccc(OCCCc2sc(N3CCCc4c3nnc(Nc3nc5ccccc5s3)c4C)nc2C(=O)[O-])c(F)c1)Cc1ccc(NC(=O)[C@H](CCCNC(N)=O)NC(=O)[C@@H](NC(=O)CCOCCN2C(=O)C=CC2=O)C(C)C)cc1] |
| L9A-P14 | 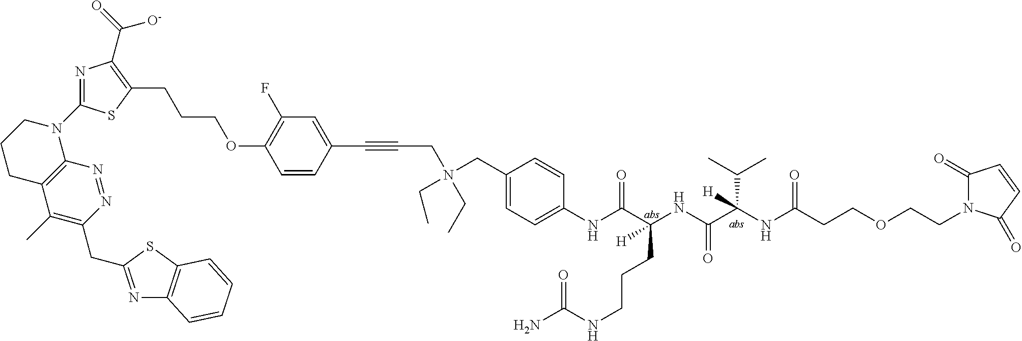 |
| L9A-P15 | 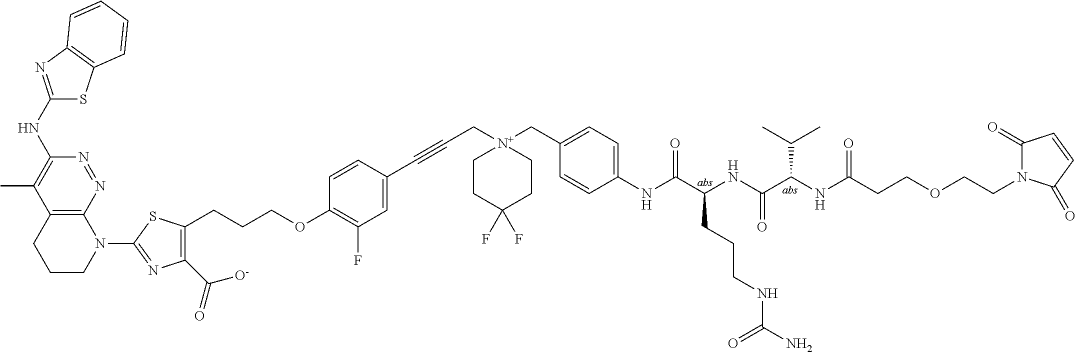 [SMILES: Cc1c(Nc2nc3ccccc3s2)nnc2c1CCCN2c1nc(C(=O)[O-])c(CCCOc2ccc(C#CC[N+]3(Cc4ccc(NC(=O)[C@H](CCCNC(N)=O)NC(=O)[C@@H](NC(=O)CCOCCN5C(=O)C=CC5=O)C(C)C)cc4)CCC(F)(F)CC3)cc2F)s1] |
| L9C-P16 | 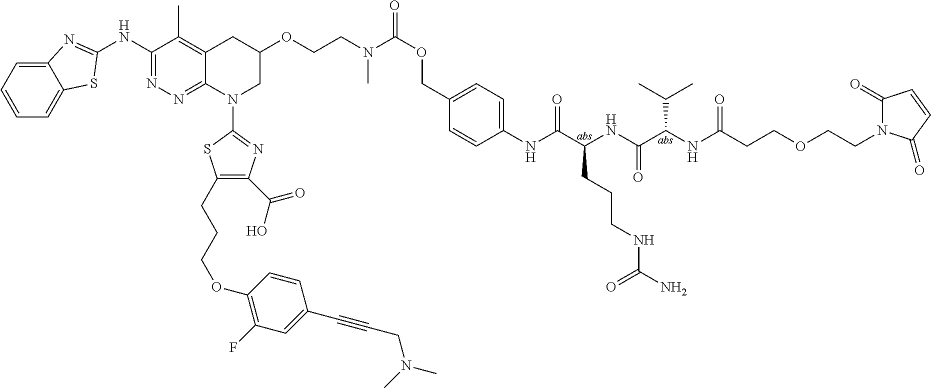 [SMILES: Cc1c(Nc2nc3ccccc3s2)nnc2c1CC(OCCN(C)C(=O)OCc1ccc(NC(=O)[C@H](CCCNC(N)=O)NC(=O)[C@@H](NC(=O)CCOCCN3C(=O)C=CC3=O)C(C)C)cc1)CN2c1nc(C(=O)O)c(CCCOc2ccc(C#CCN(C)C)cc2F)s1] |
| L9A-P1 | 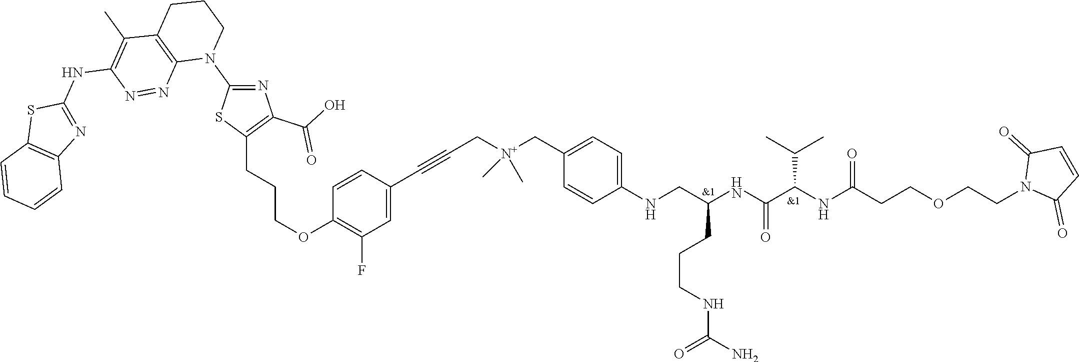 [SMILES: Cc1c(Nc2nc3ccccc3s2)nnc2c1CCCN2c1nc(C(=O)O)c(CCCOc2ccc(C#CC[N+](C)(C)Cc3ccc(NC[C@H](CCCNC(N)=O)NC(=O)[C@@H](NC(=O)CCOCCN4C(=O)C=CC4=O)C(C)C)cc3)cc2F)s1] |
| L9C-P17 | 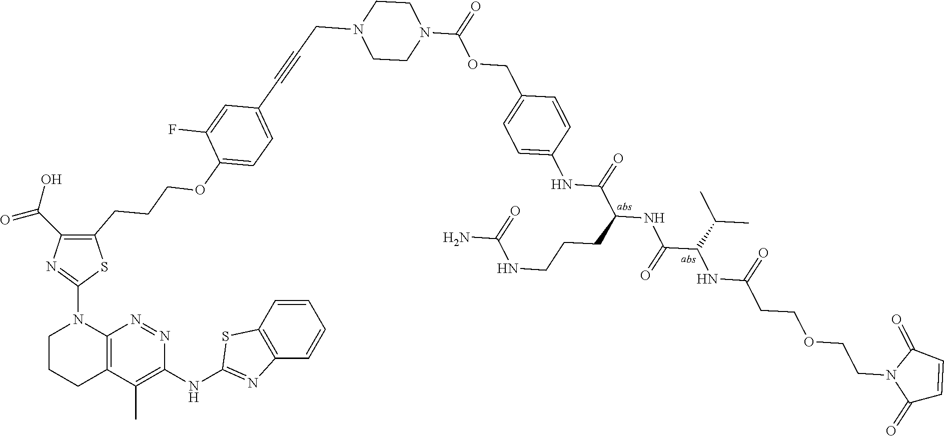 |
| L9A-P18 | 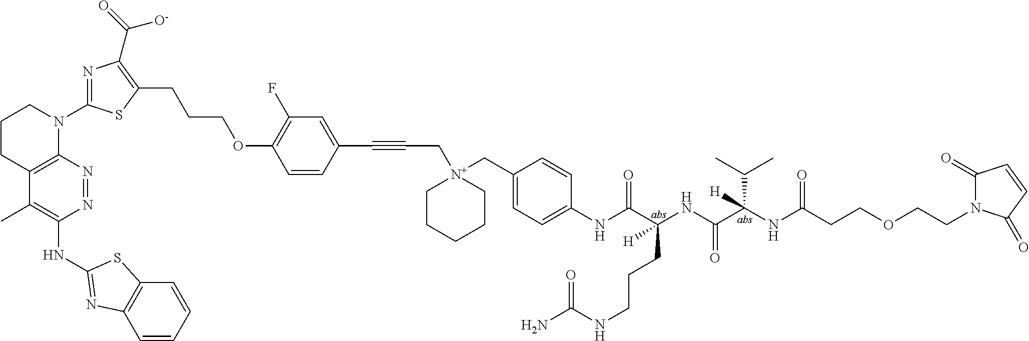 [SMILES: Cc1c(Nc2nc3ccccc3s2)nnc2c1CCCN2c1nc(C(=O)[O-])c(CCCOc2ccc(C#CC[N+]3(Cc4ccc(NC(=O)[C@H](CCCNC(N)=O)NC(=O)[C@@H](NC(=O)CCOCCN5C(=O)C=CC5=O)C(C)C)cc4)CCCCC3)cc2F)s1] |
| L9C-P19 | 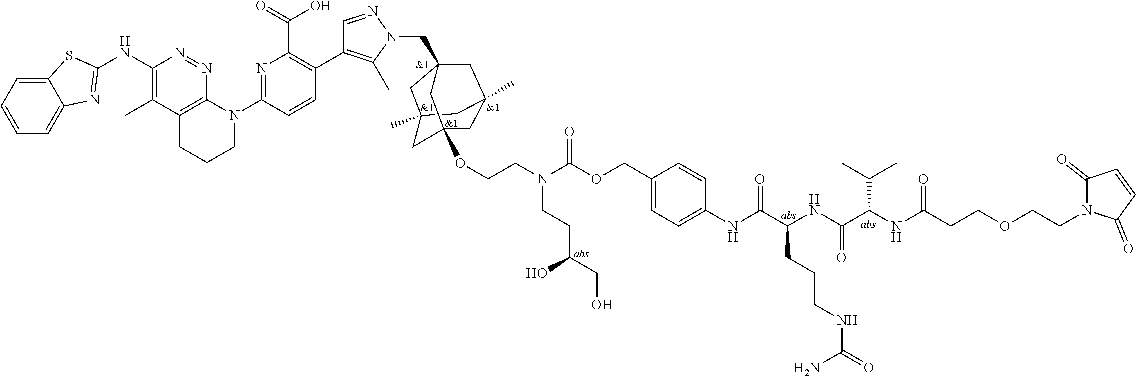 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P20 | 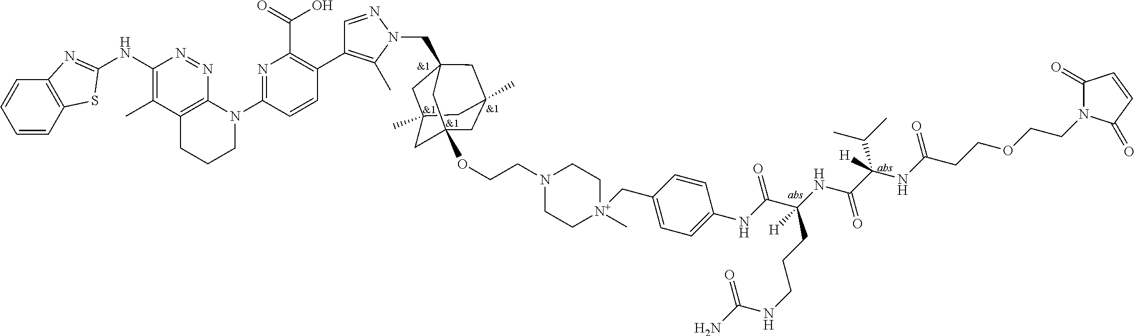 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CC[N+](C)(Cc6ccc(NC(=O)[C@H](CCCNC(N)=O)NC(=O)[C@@H](NC(=O)CCOCCN7C(=O)C=CC7=O)C(C)C)cc6)CC3)(C5)C4)c2C)c(C(=O)O)n1] |
| L9A-P21 | 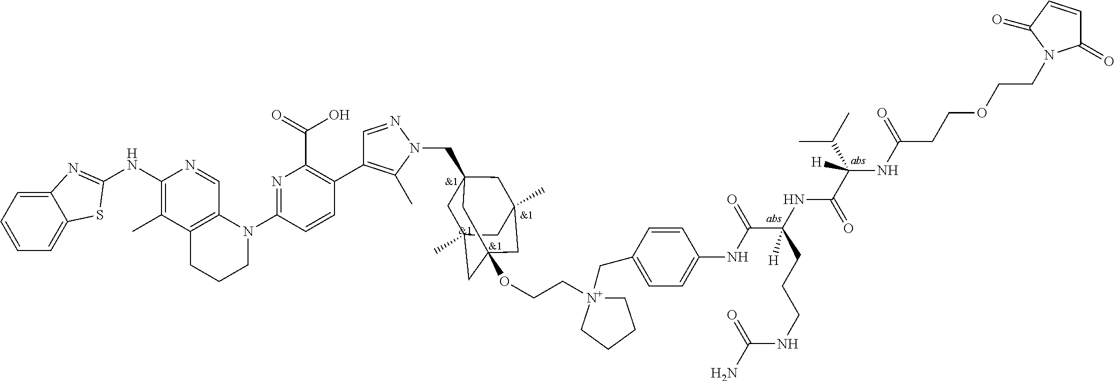 [SMILES: Cc1c(Nc2nc3ccccc3s2)ncc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(C(=O)O)n1] |
| L9C-P22 | 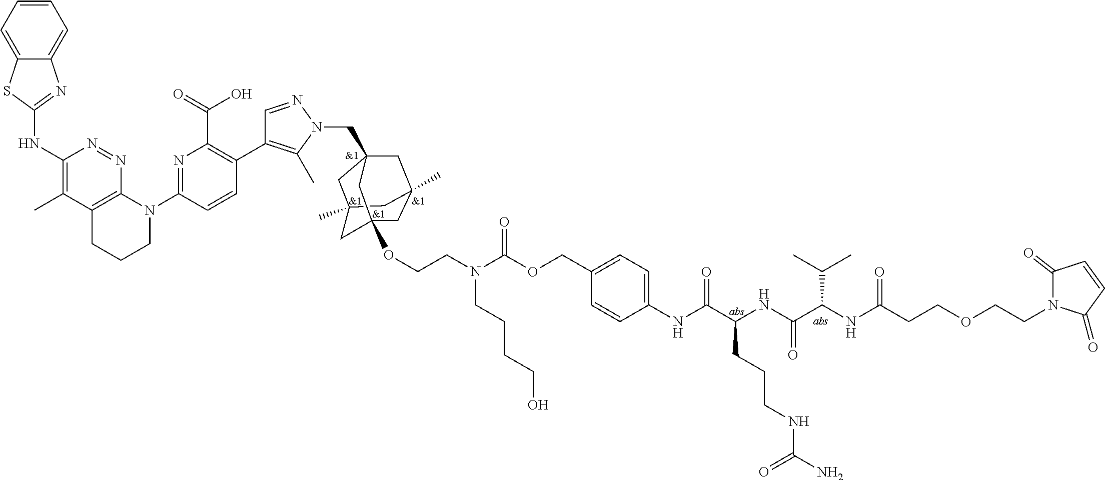 |
| L9C-P23 | 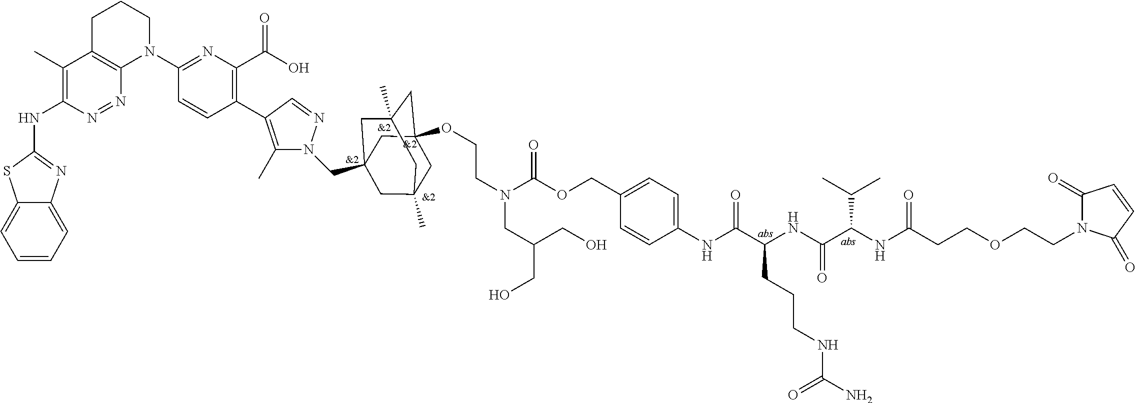 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CC(CO)CO)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P24 | 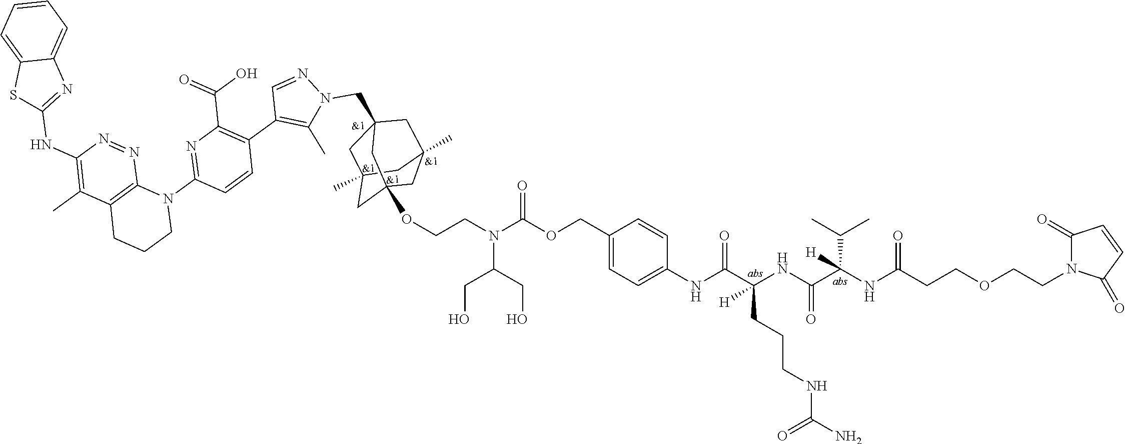 [SMILES: Cc1c(Nc2nc3ccccc3s2)nnc2c1CCCN2c1ccc(-c2cnn(C[C@]34C[C@]5(C)C[C@](C)(C3)C[C@@](OCCN(C(=O)OCc3ccc(NC(=O)[C@H](CCCNC(N)=O)NC(=O)[C@@H](NC(=O)CCOCCN6C(=O)C=CC6=O)C(C)C)cc3)C(CO)CO)(C5)C4)c2C)c(C(=O)O)n1] |
| L9A-P2 | 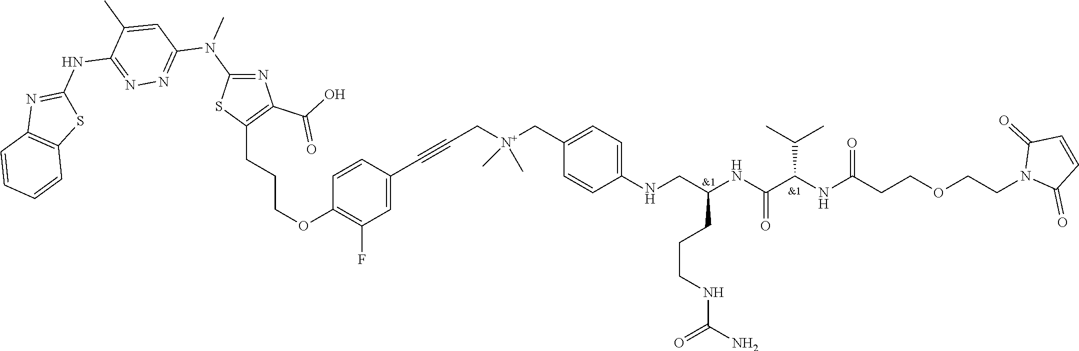 [SMILES: Cc1cc(N(C)c2nc(C(=O)O)c(CCCOc3ccc(C#CC[N+](C)(C)Cc4ccc(NC[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P25 | 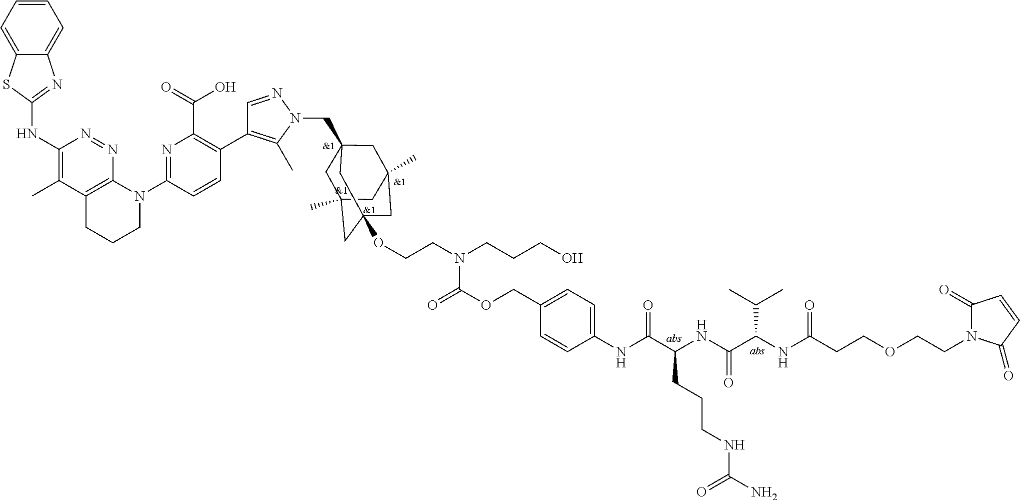 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P26 | 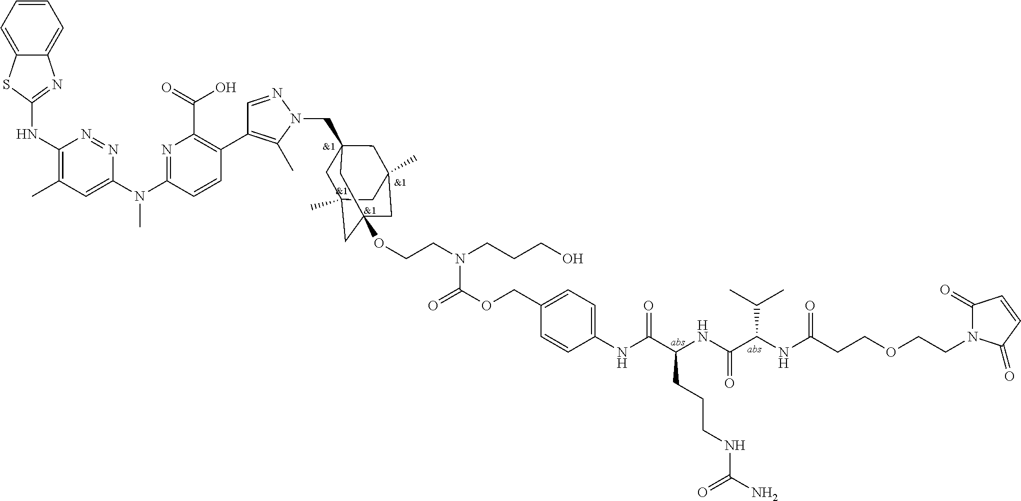 [SMILES: Cc1cc(N(C)c2ccc(-c3cnn(C[C@]45C[C@]6(C)C[C@](C)(C4)C[C@@](OCCN(CCCO)C(=O)OCc4ccc(NC(=O)[C@H](CCCNC(N)=O)NC(=O)[C@@H](NC(=O)CCOCCN7C(=O)C=CC7=O)C(C)C)cc4)(C6)C5)c3C)c(C(=O)O)n2)nnc1Nc1nc2ccccc2s1] |
| L9A-P27 | 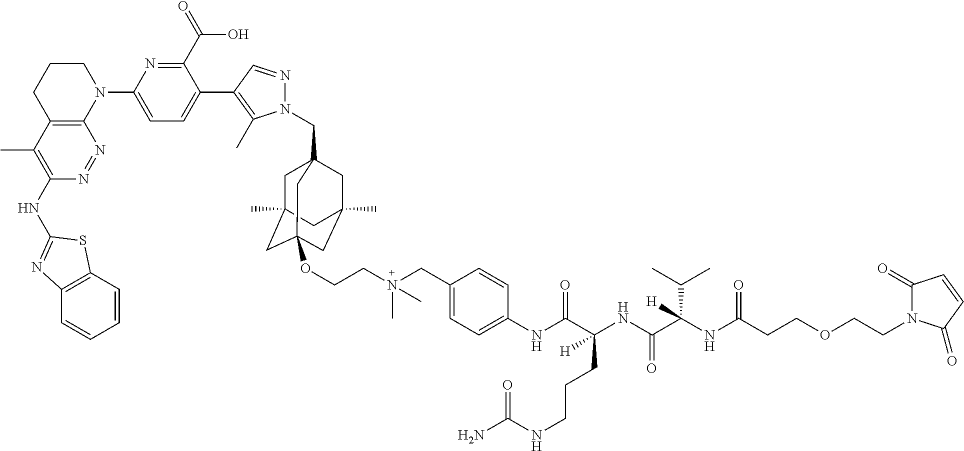 |
| L9A-P28 | 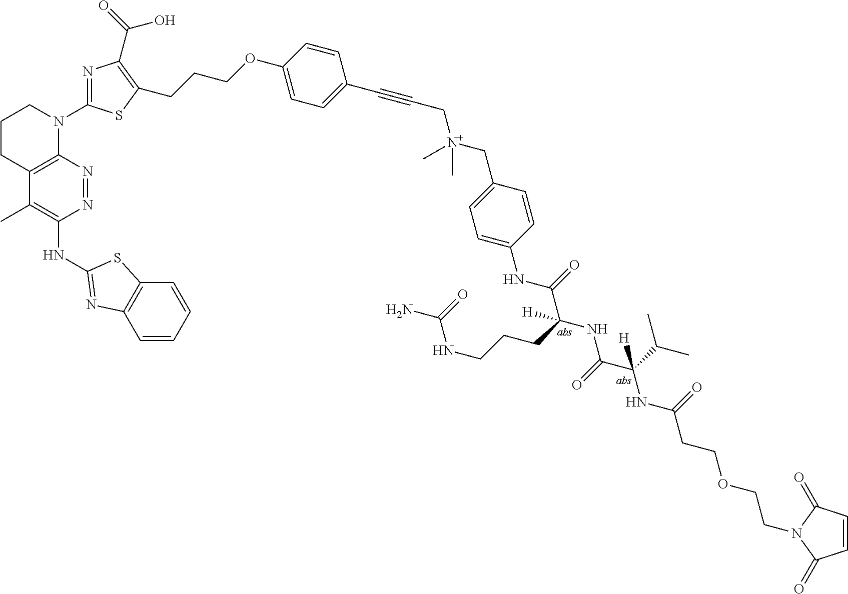 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3)cc2)s1] |
| L9C-P29 | 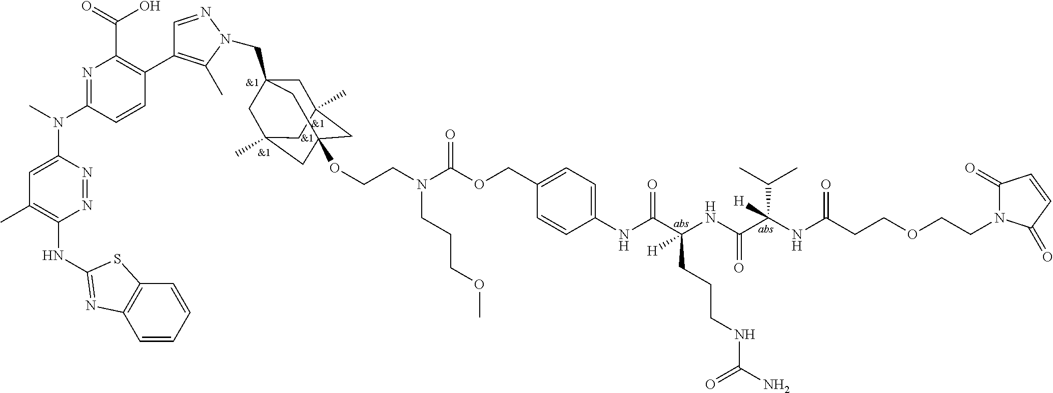 [SMILES: COCCCN(CCO[C@]12C[C@@]3(C)C[C@](C)(C[C@](Cn4ncc(-c5ccc(N(C)c6cc(C)c(Nc7nc8ccccc8s7)nn6)nc5C(=O)O)c4C)(C3)C1)C2)C(=O)OCc1ccc(NC(=O)[C@H](CCCNC(N)=O)NC(=O)[C@@H](NC(=O)CCOCCN2C(=O)C=CC2=O)C(C)C)cc1] |
| L9A-P30 | 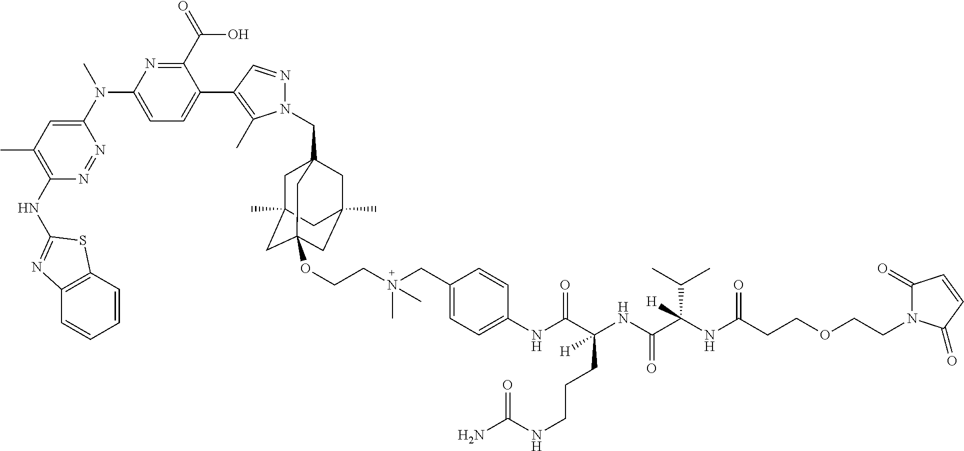 |
| L9C-P31 | 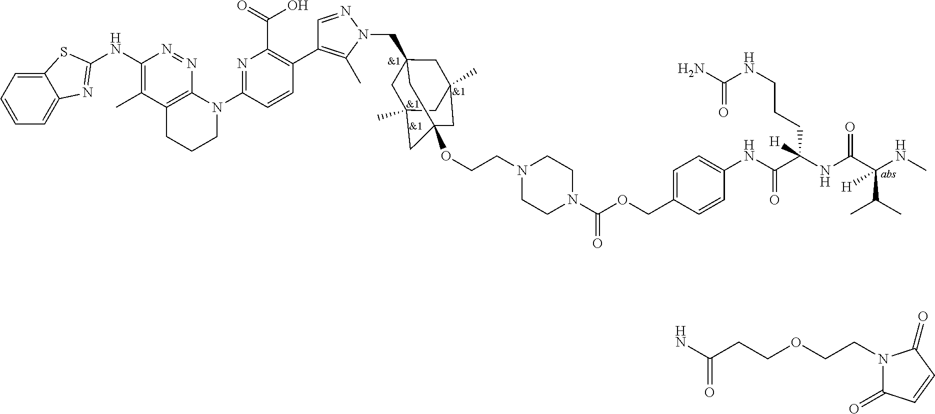 |
| L9A-P32 | 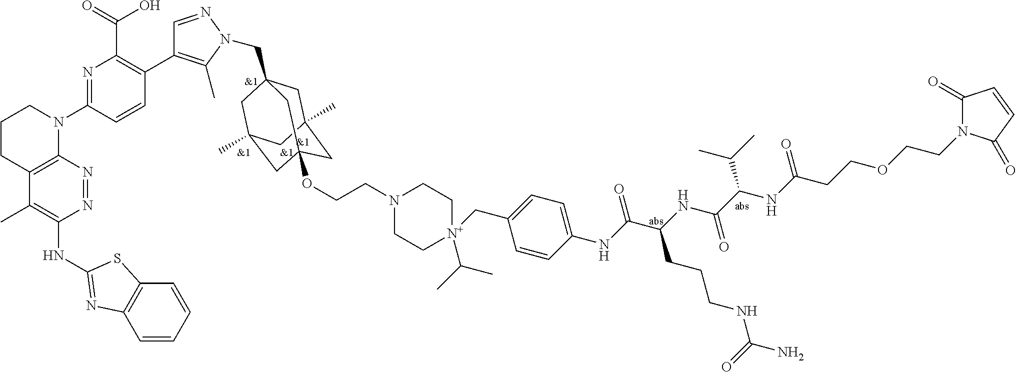 [SMILES: Cc1c(Nc2nc3ccccc3s2)nnc2c1CCCN2c1ccc(-c2cnn(C[C@]34C[C@]5(C)C[C@](C)(C3)C[C@@](OCCN3CC[N+](Cc6ccc(NC(=O)[C@H](CCCNC(N)=O)NC(=O)[C@@H](NC(=O)CCOCCN7C(=O)C=CC7=O)C(C)C)cc6)(C(C)C)CC3)(C5)C4)c2C)c(C(=O)O)n1] |
| L9A-P33 | 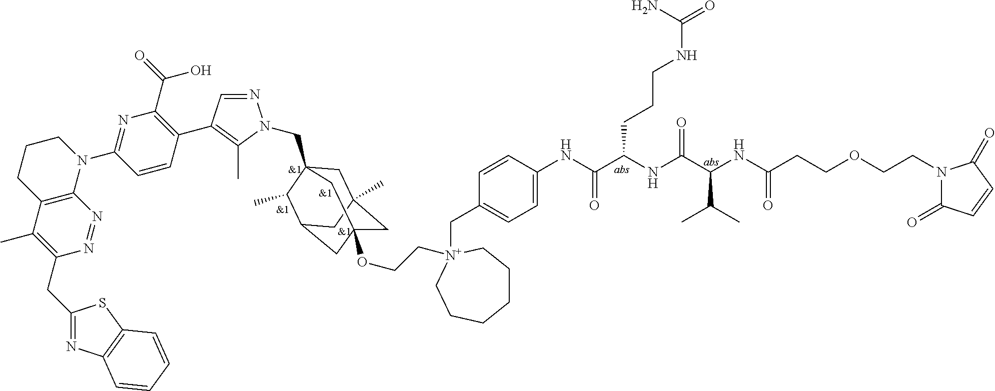 [SMILES: Cc1c(Cc2nc3ccccc3s2)nnc2c1CCCN2c1ccc(-c2cnn(C[C@@]34C[C@]5(C)CC(C[C@@](OCC[N+]6(Cc7ccc(NC(=O)[C@H](CCCNC(N)=O)NC(=O)[C@@H](NC(=O)CCOCCN8C(=O)C=CC8=O)C(C)C)cc7)CCCCCC6)(C5)C3)[C@@H]4C)c2C)c(C(=O)O)n1] |
| L9A-P34 | 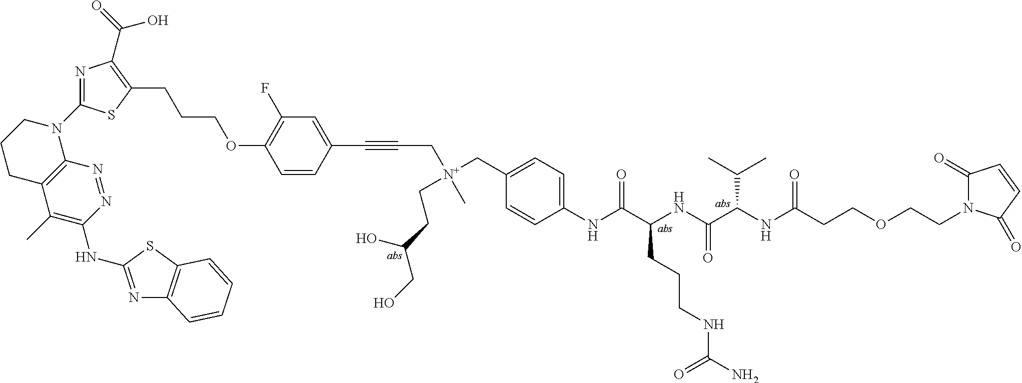 |
| L9A-P35 | 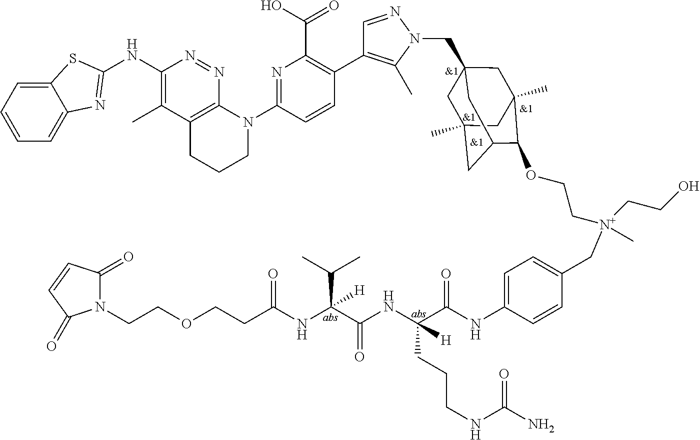 [SMILES: Cc1c(Nc2nc3ccccc3s2)nnc2c1CCCN2c1ccc(-c2cnn(C[C@@]34CC5C[C@@](C)(C3)C[C@@](C)(C4)[C@H]5OCC[N+](C)(CCO)Cc3ccc(NC(=O)[C@H](CCCNC(N)=O)NC(=O)[C@@H](NC(=O)CCOCCN4C(=O)C=CC4=O)C(C)C)cc3)c2C)c(C(=O)O)n1] |
| L9A-P36 | 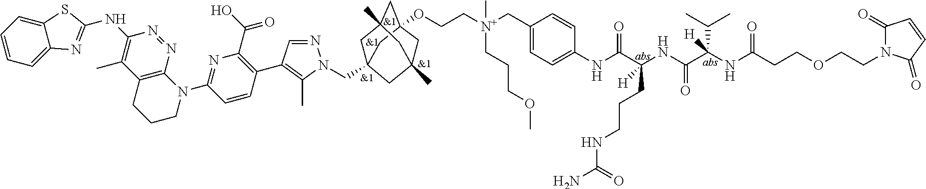 [SMILES: COCCC[N+](C)(CCO[C@]12C[C@@]3(C)C[C@](C)(C[C@](Cn4ncc(-c5ccc(N6CCCc7c6nnc(Nc6nc8ccccc8s6)c7C)nc5C(=O)O)c4C)(C3)C1)C2)Cc1ccc(NC(=O)[C@H](CCCNC(N)=O)NC(=O)[C@@H](NC(=O)CCOCCN2C(=O)C=CC2=O)C(C)C)cc1] |
| L9A-P37 | 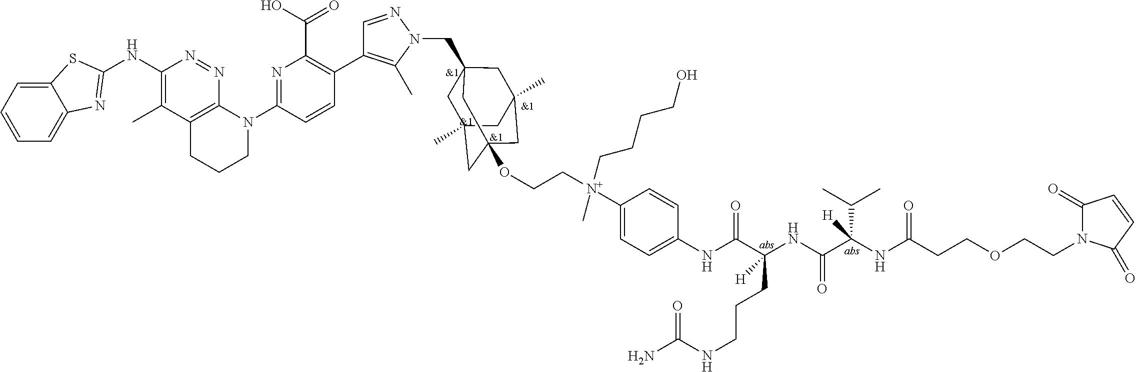 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CCCCO)c3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P38 | 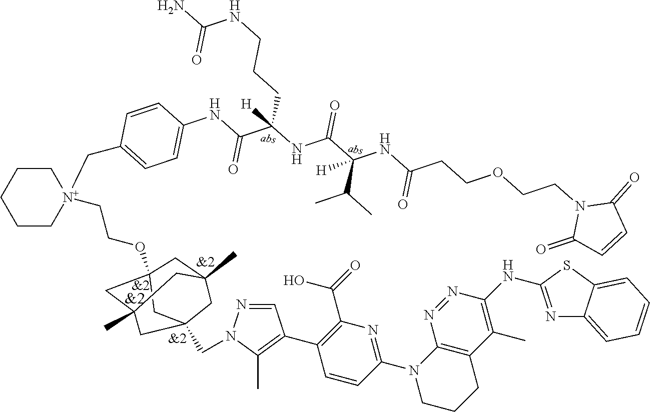 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCCC3)(C5)C4)c2C)c(C(=O)O)n1] |
| L9A-P39 | 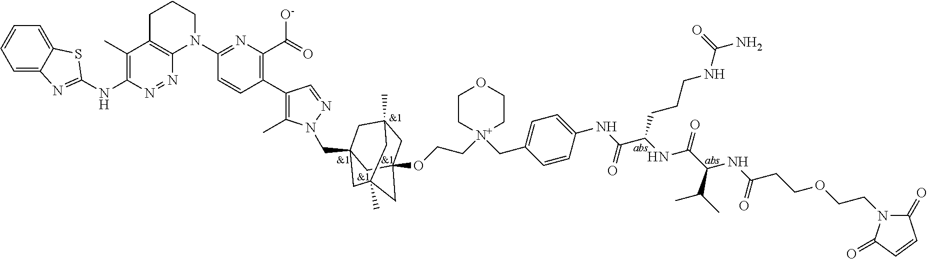 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCOCC3)(C5)C4)c2C)c(C(=O)[O-])n1] |
| L9C-P40 | 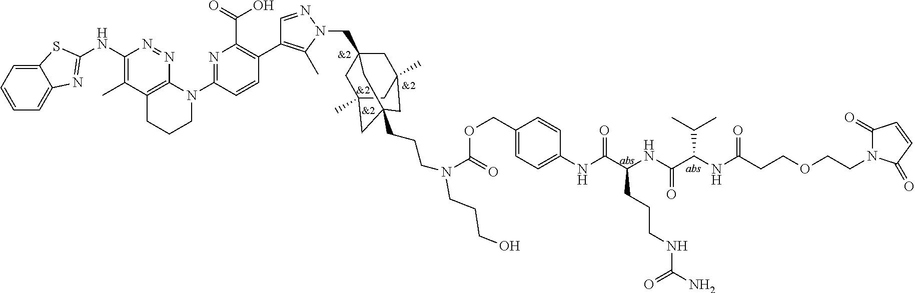 [SMILES: Cc1c(Nc2nc3ccccc3s2)nnc2c1CCCN2c1ccc(-c2cnn(C[C@]34C[C@@]5(C)C[C@](C)(C[C@](CCCN(CCCO)C(=O)OCc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9A-P41 | 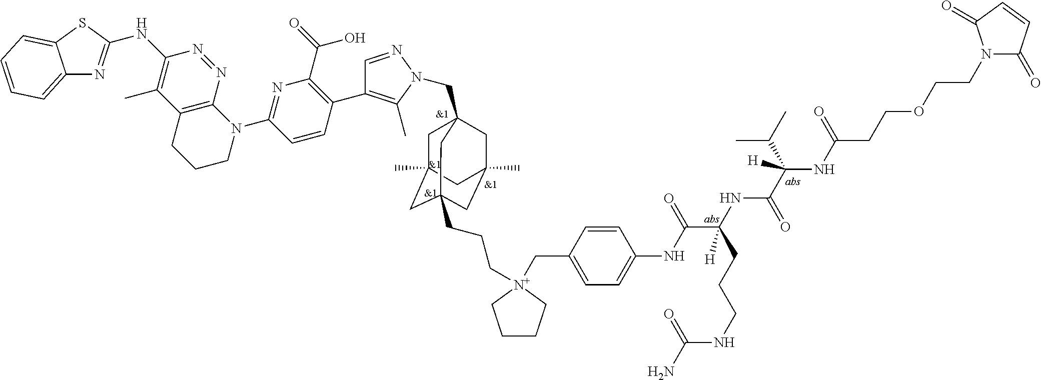 [SMILES: Cc1c(Nc2nc3ccccc3s2)nnc2c1CCCN2c1ccc(-c2cnn(C[C@]34C[C@@]5(C)C[C@](C)(C[C@](CCC[N+]6(Cc7ccc(NC(=O)[C@H](CCCNC(N)=O)NC(=O)[C@@H](NC(=O)CCOCCN8C(=O)C=CC8=O)C(C)C)cc7)CCCC6)(C5)C3)C4)c2C)c(C(=O)O)n1] |
| L9A-P42 | 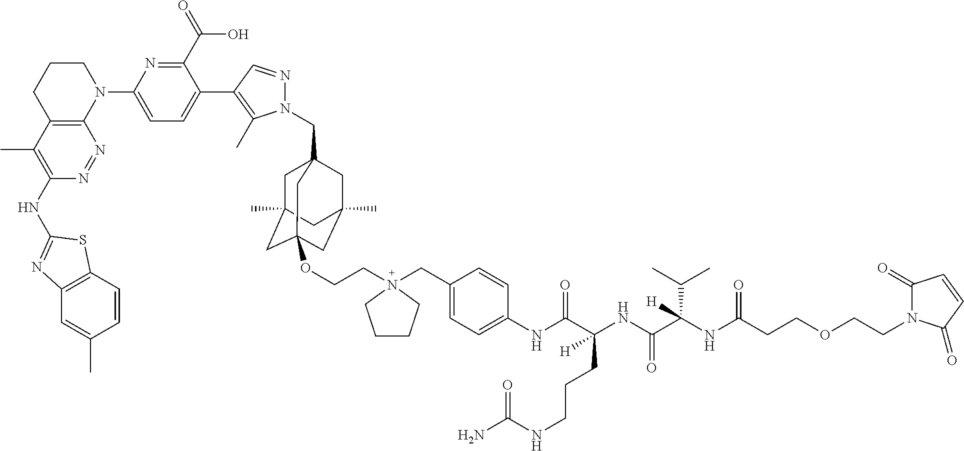 |
| L9A-P43 | 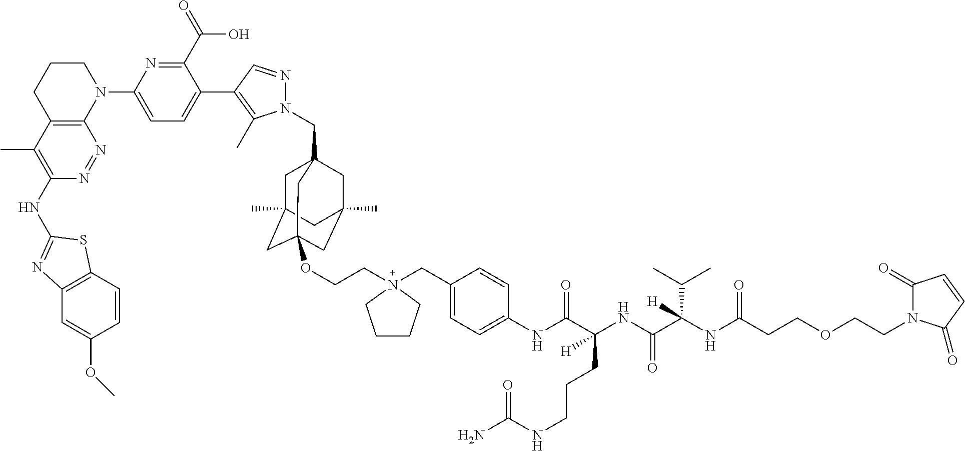 |
| L9C-P44 | 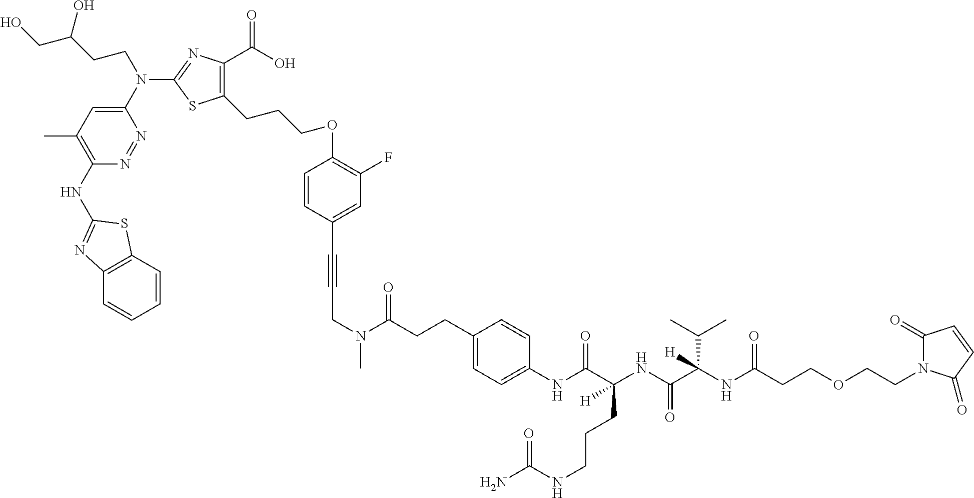 [SMILES: Cc1cc(N(CCC(O)CO)c2nc(C(=O)O)c(CCCOc3ccc(C#CCN(C)C(=O)CCc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P45 | 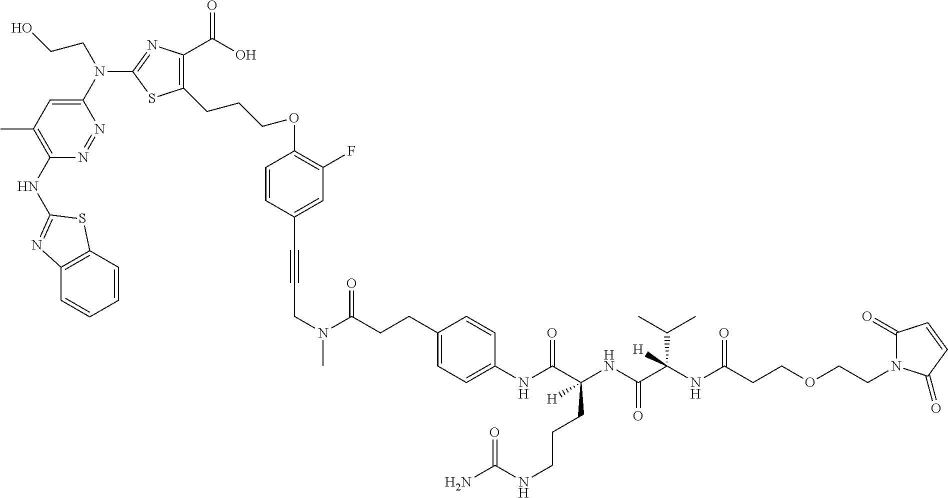 [SMILES: Cc1cc(N(CCO)c2nc(C(=O)O)c(CCCOc3ccc(C#CCN(C)C(=O)CCc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P46 | 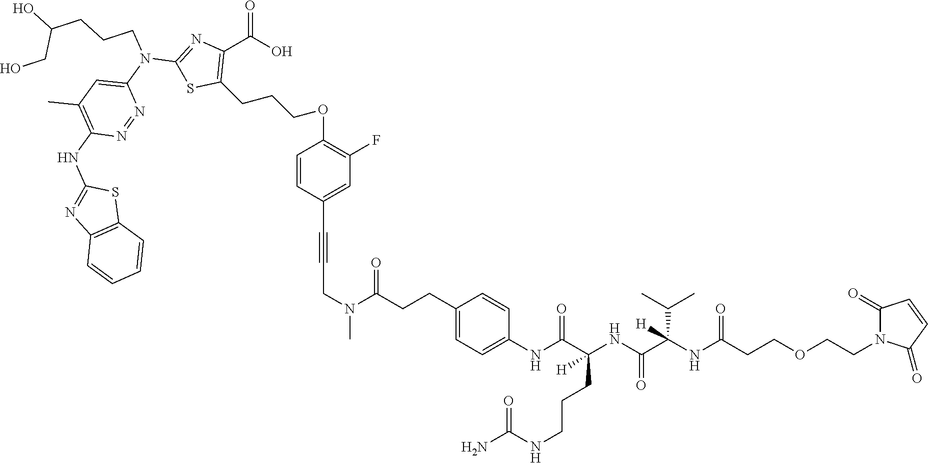 [SMILES: Cc1cc(N(CCCC(O)CO)c2nc(C(=O)O)c(CCCOc3ccc(C#CCN(C)C(=O)CCc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P4 | 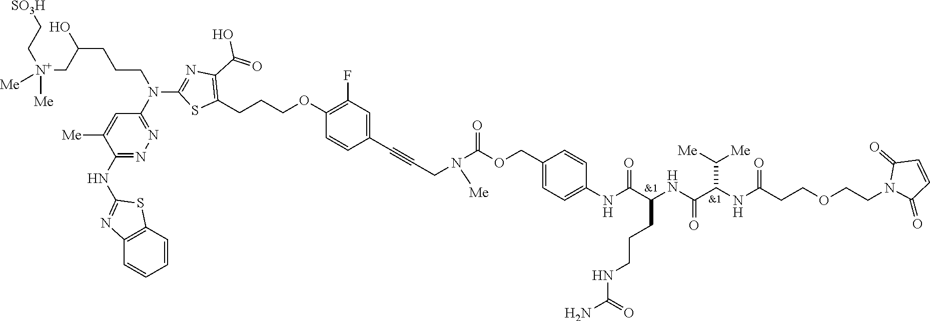 [SMILES: Cc1cc(N(CCCC(O)C[N+](C)(C)CCS(=O)(=O)O)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P5 | 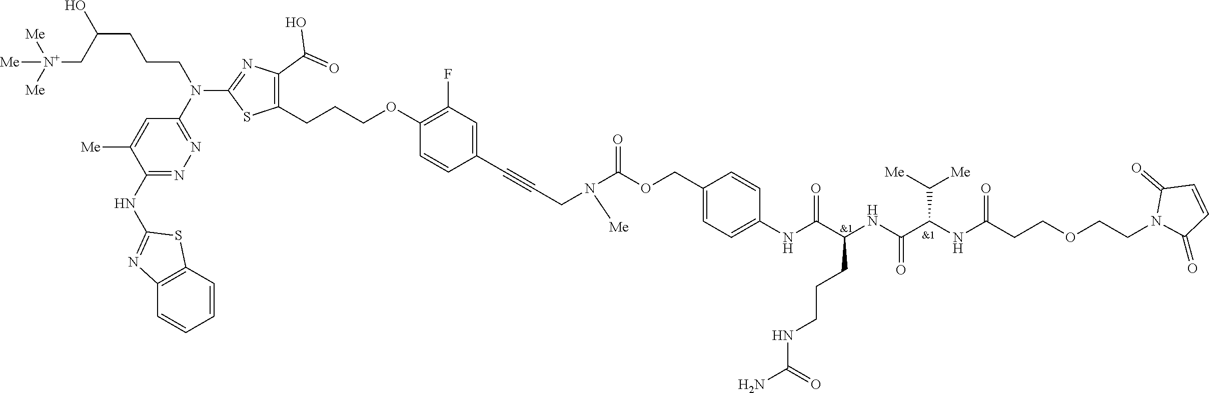 [SMILES: Cc1cc(N(CCCC(O)C[N+](C)(C)C)c2nc(C(=O)O)c(CCCOc3ccc(C#CCN(C)C(=O)OCc4ccc(NC(=O)[C@H](CCCNC(N)=O)NC(=O)[C@@H](NC(=O)CCOCCN5C(=O)C=CC5=O)C(C)C)cc4)cc3F)s2)nnc1Nc1nc2ccccc2s1] |
| L9C-P59 | 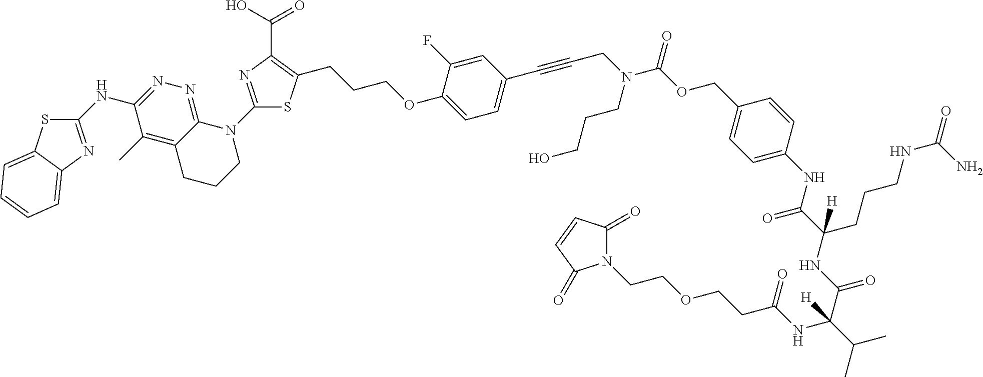 [SMILES: Cc1c(Nc2nc3ccccc3s2)nnc2c1CCCN2c1nc(C(=O)O)c(CCCOc2ccc(C#CCN(CCCO)C(=O)OCc3ccc(NC(=O)[C@H](CCCNC(N)=O)NC(=O)[C@@H](NC(=O)CCOCCN4C(=O)C=CC4=O)C(C)C)cc3)cc2F)s1] |
| L9C-P3 | 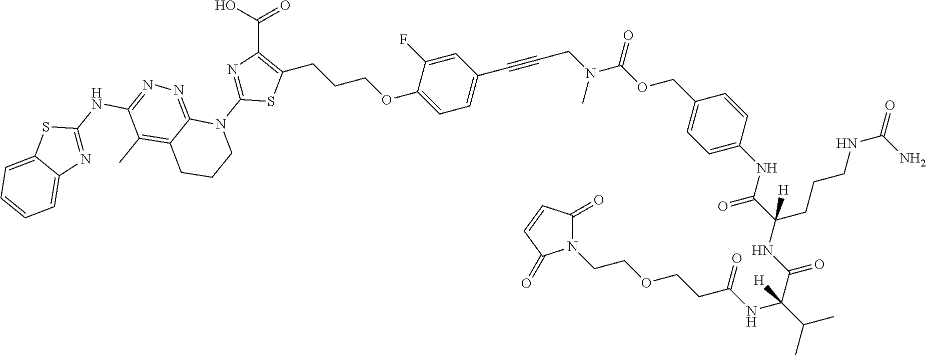 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3)cc2F)s1] |
| L9C-P60 | 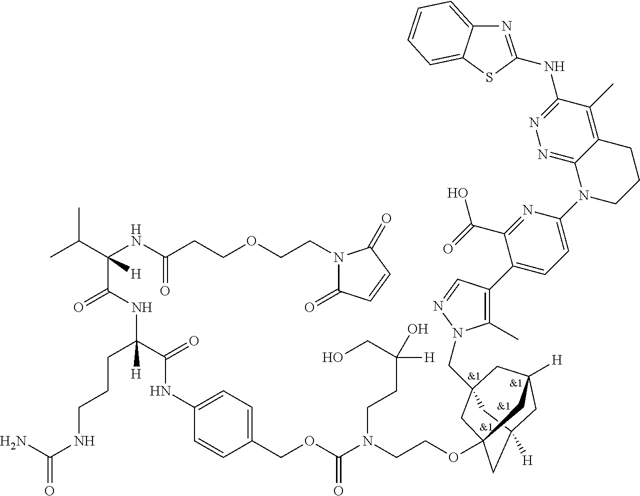 [SMILES: Cc1c(Nc2nc3ccccc3s2)nnc2c1CCCN2c1ccc(-c2cnn(C[C@@]34C[C@H]5C[C@@H](C3)C[C@](OCCN(CCC(O)CO)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P61 | 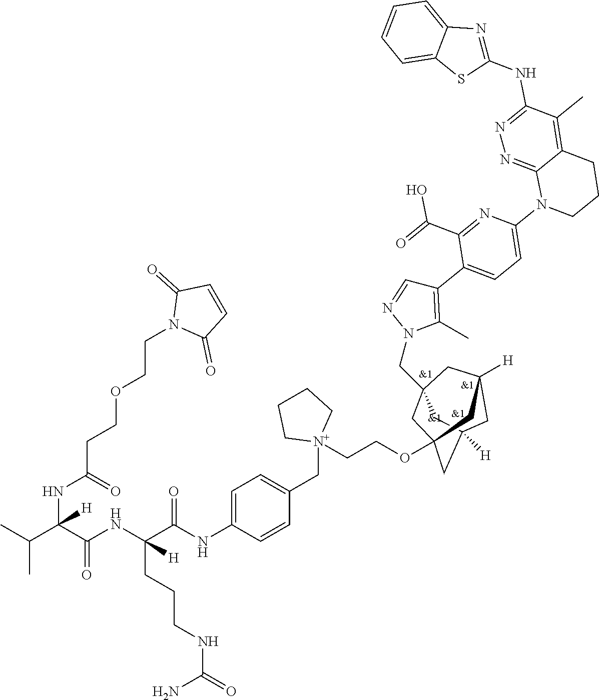 [SMILES: Cc1c(Nc2nc3ccccc3s2)nnc2c1CCCN2c1ccc(-c2cnn(C[C@@]34C[C@H]5C[C@@H](C3)C[C@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(C(=O)O)n1] |
| L9A-P62 | 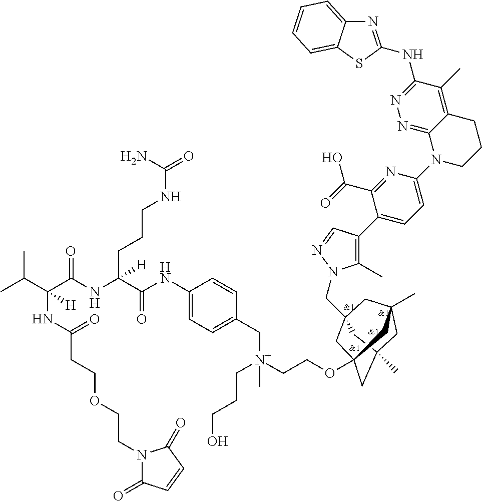 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CCCO)Cc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P63 | 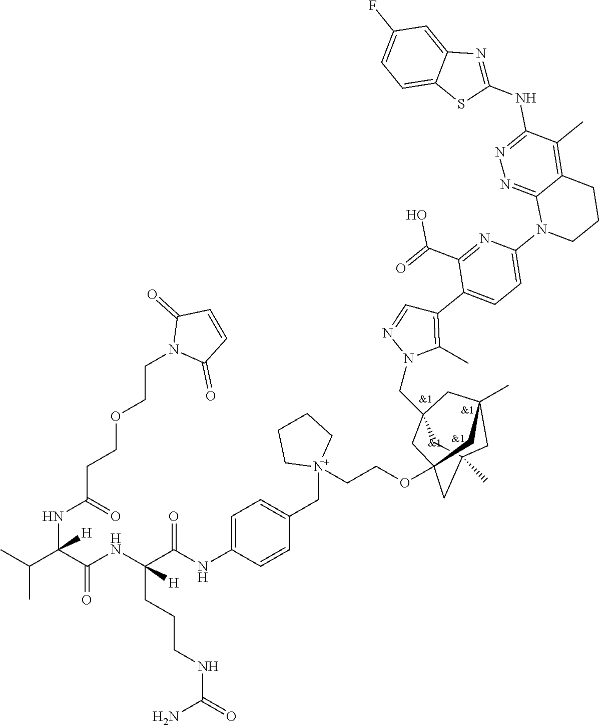 [SMILES: Cc1c(Nc2nc3cc(F)ccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(C(=O)O)n1] |
| L9A-P64 | 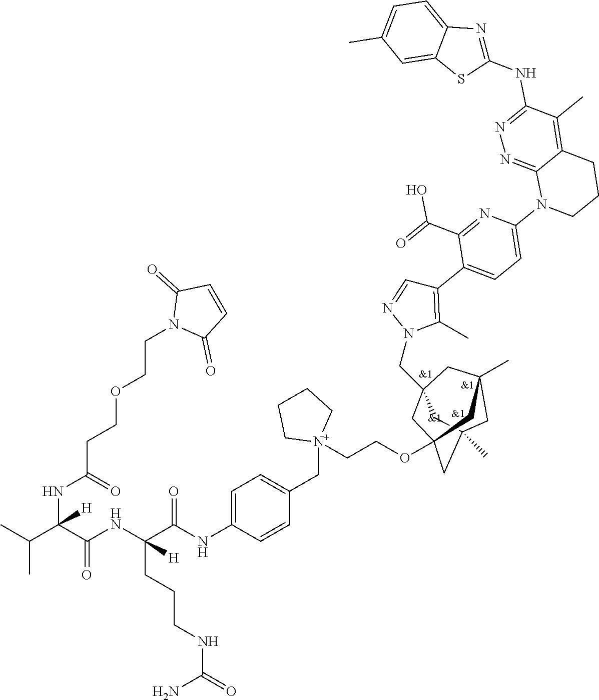 [SMILES: Cc1ccc2nc(Nc3nnc4c(c3C)CCCN4c3ccc(-c4cnn(C[C@]56C[C@]7(C)C[C@](C)(C5)C[C@@](OCC[N+]5(Cc8ccc(NC(=O)[C@H](CCCNC(N)=O)NC(=O)[C@@H](NC(=O)CCOCCN9C(=O)C=CC9=O)C(C)C)cc8)CCCC5)(C7)C6)c4C)c(C(=O)O)n3)sc2c1] |
| L9A-P65 | 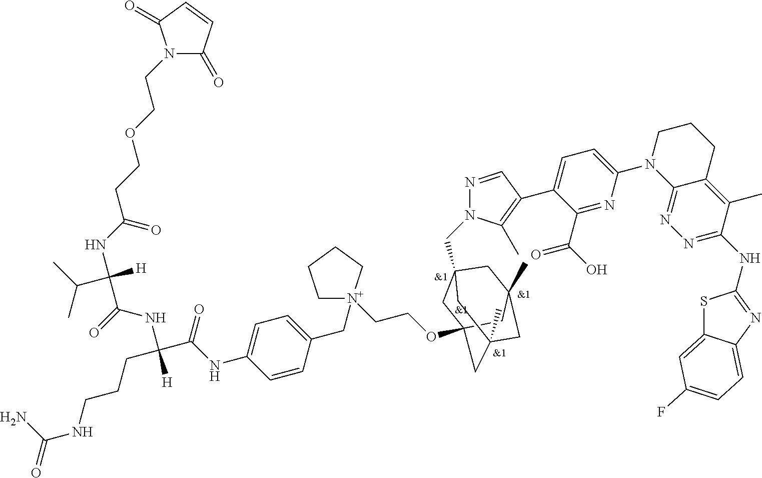 [SMILES: Cc1c(Nc2nc3ccc(F)cc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(C(=O)O)n1] |
| L9A-P66 | 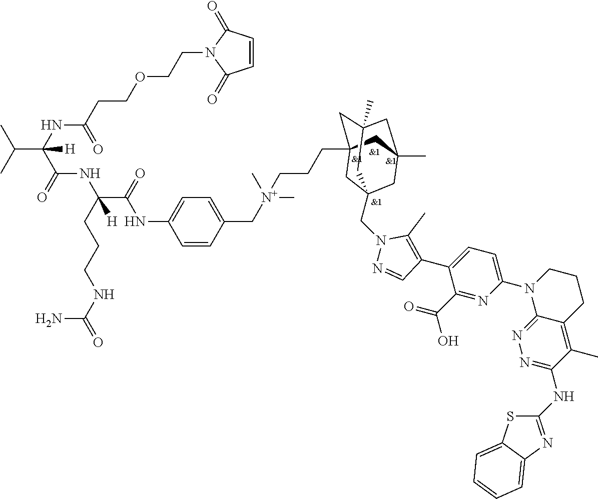 [SMILES: Cc1c(Nc2nc3ccccc3s2)nnc2c1CCCN2c1ccc(-c2cnn(C[C@]34C[C@@]5(C)C[C@](C)(C[C@](CCC[N+](C)(C)Cc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9A-P67 | 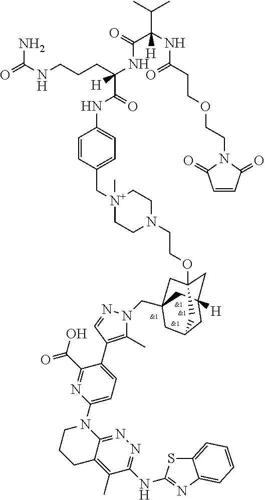 [SMILES: Cc1c(Nc2nc3ccccc3s2)nnc2c1CCCN2c1ccc(-c2cnn(C[C@@]34C[C@H]5C[C@@H](C3)C[C@](OCCN3CC[N+](C)(Cc6ccc(NC(=O)[C@H](CCCNC(N)=O)NC(=O)[C@@H](NC(=O)CCOCCN7C(=O)C=CC7=O)C(C)C)cc6)CC3)(C5)C4)c2C)c(C(=O)O)n1] |
| L9A-P68 | 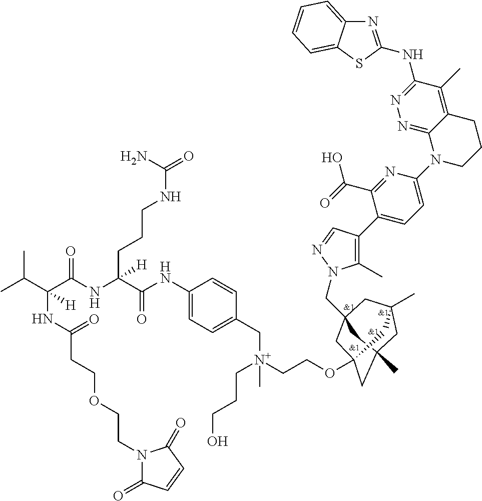 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CCCO)Cc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P69 | 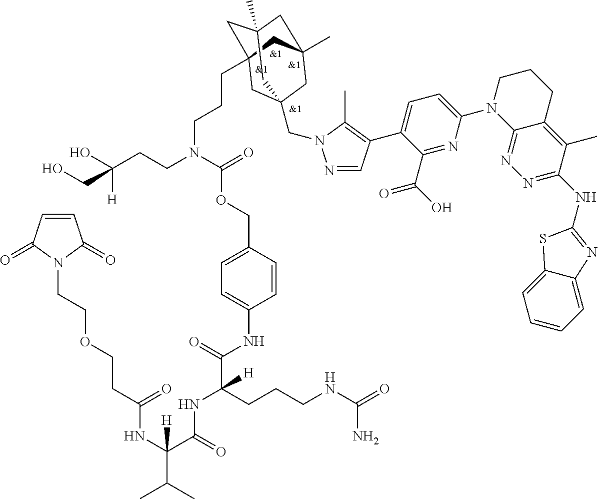 [SMILES: Cc1c(Nc2nc3ccccc3s2)nnc2c1CCCN2c1ccc(-c2cnn(C[C@]34C[C@@]5(C)C[C@](C)(C[C@](CCCN(CC[C@H](O)CO)C(=O)OCc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9A-P48 | 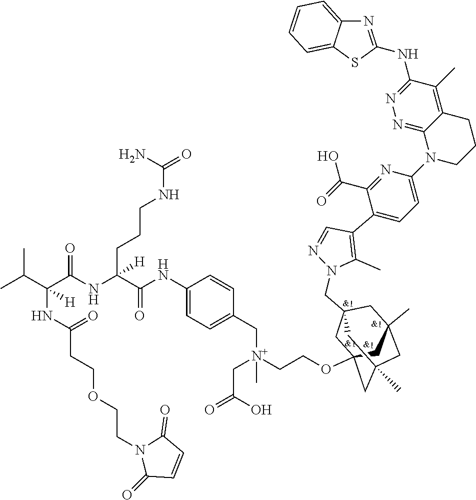 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CC(=O)O)Cc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P70 | 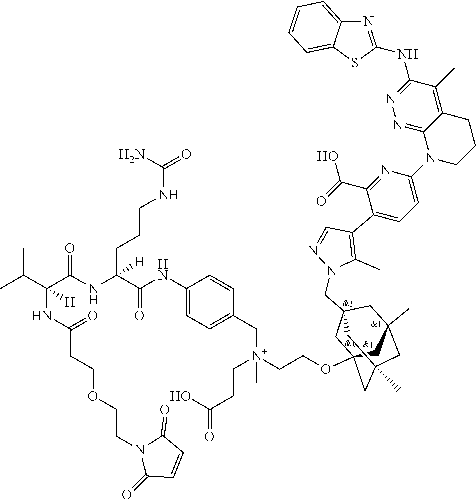 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CCC(=O)O)Cc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P71 | 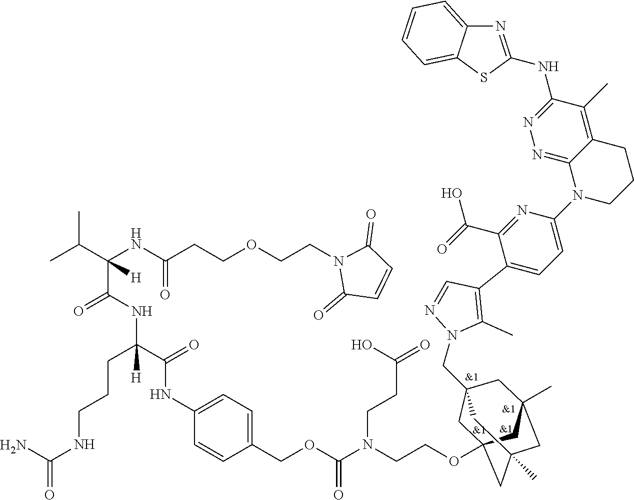 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CCC(=O)O)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P72 | 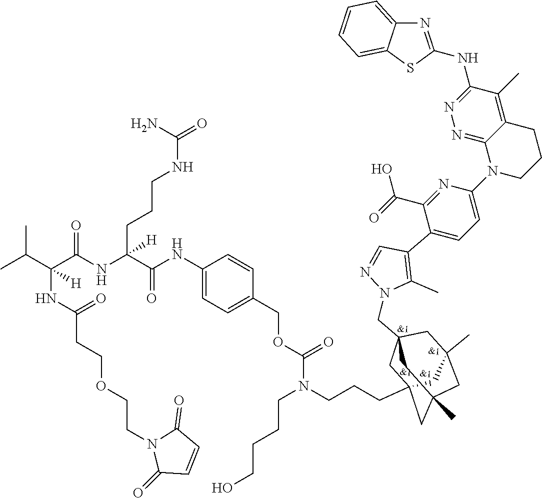 [SMILES: Cc1c(Nc2nc3ccccc3s2)nnc2c1CCCN2c1ccc(-c2cnn(C[C@]34C[C@@]5(C)C[C@](C)(C[C@](CCCN(CCCCO)C(=O)OCc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9A-P49 | 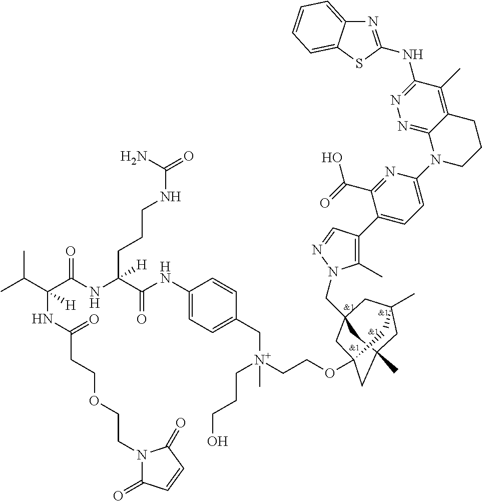 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CCCO)Cc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P51 | 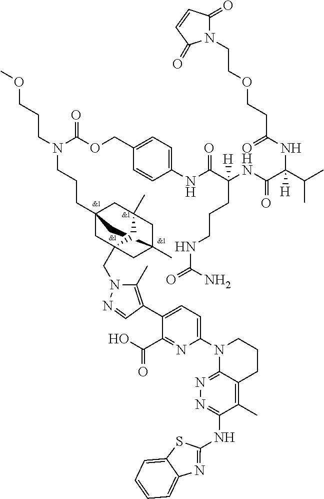 [SMILES: COCCCN(CCC[C@]12C[C@]3(C)C[C@](C)(C1)C[C@@](Cn1ncc(-c4ccc(N5CCCc6c5nnc(Nc5nc7ccccc7s5)c6C)nc4C(=O)O)c1C)(C3)C2)C(=O)OCc1ccc(NC(=O)[C@H](CCCNC(N)=O)NC(=O)[C@@H](NC(=O)CCOCCN2C(=O)C=CC2=O)C(C)C)cc1] |
| L9A-P50 | 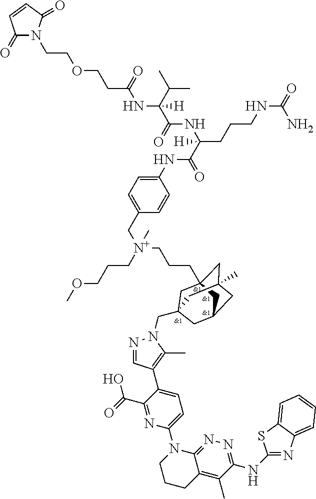 [SMILES: COCCC[N+](C)(CCC[C@]12C[C@@H]3C[C@](C)(C1)C[C@@](Cn1ncc(-c4ccc(N5CCCc6c5nnc(Nc5nc7ccccc7s5)c6C)nc4C(=O)O)c1C)(C3)C2)Cc1ccc(NC(=O)[C@H](CCCNC(N)=O)NC(=O)[C@@H](NC(=O)CCOCCN2C(=O)C=CC2=O)C(C)C)cc1] |
| L9A-P52 | 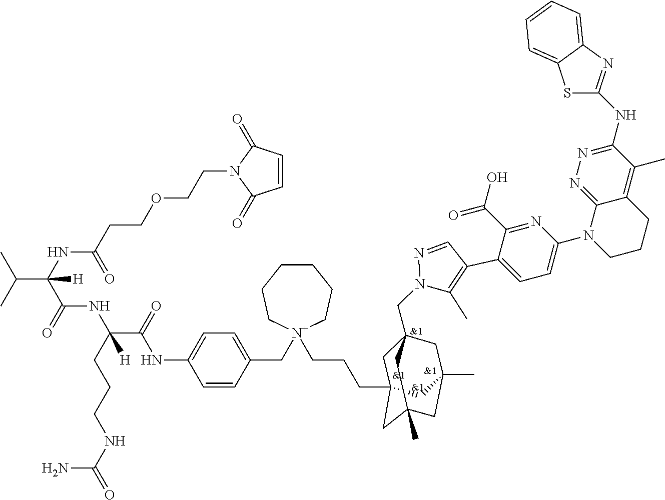 [SMILES: Cc1c(Nc2nc3ccccc3s2)nnc2c1CCCN2c1ccc(-c2cnn(C[C@]34C[C@@]5(C)C[C@](C)(C[C@](CCC[N+]6(Cc7ccc(NC(=O)[C@H](CCCNC(N)=O)NC(=O)[C@@H](NC(=O)CCOCCN8C(=O)C=CC8=O)C(C)C)cc7)CCCCCC6)(C5)C3)C4)c2C)c(C(=O)O)n1] |
| L9C-P53 | 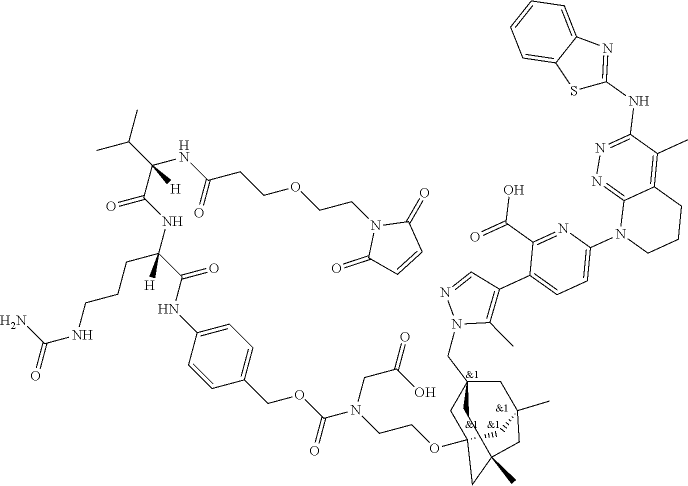 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CC(=O)O)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P55 | 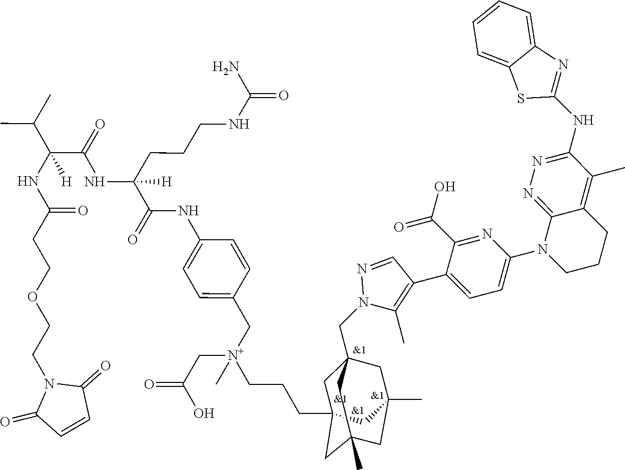 [SMILES: Cc1c(Nc2nc3ccccc3s2)nnc2c1CCCN2c1ccc(-c2cnn(C[C@]34C[C@@]5(C)C[C@](C)(C[C@](CCC[N+](C)(CC(=O)O)Cc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9C-P54 | 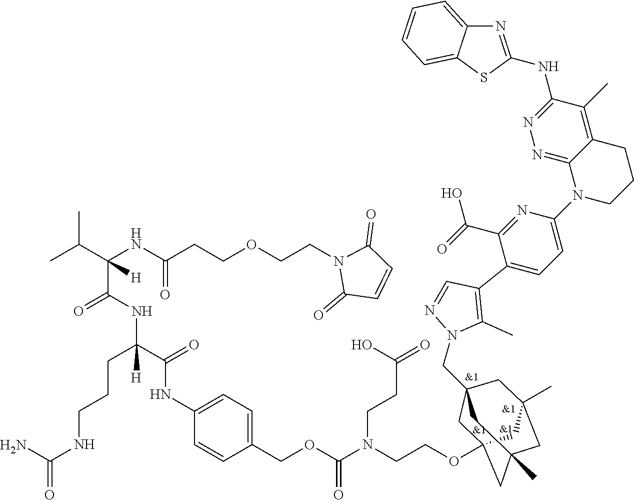 [SMILES: Cc1c(Nc2nc3ccccc3s2)nnc2c1CCCN2c1ccc(-c2cnn(C[C@]34C[C@]5(C)C[C@](C)(C3)C[C@@](OCCN(CCC(=O)O)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9C-P47 | 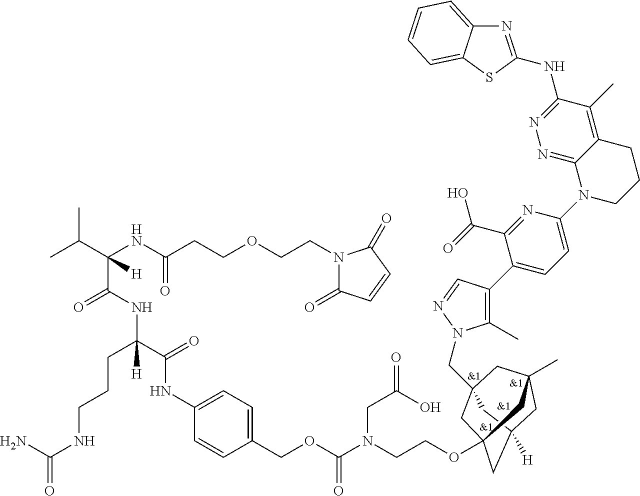 [SMILES: Cc1c(Nc2nc3ccccc3s2)nnc2c1CCCN2c1ccc(-c2cnn(C[C@@]34C[C@H]5C[C@@](C)(C3)C[C@](OCCN(CC(=O)O)C(=O)OCc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P56 | 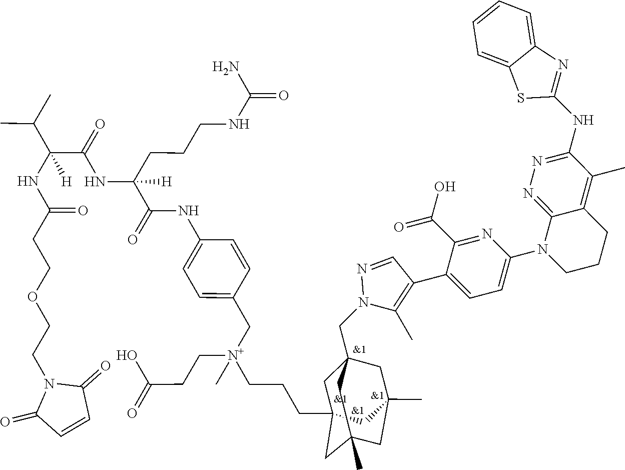 [SMILES: Cc1c(Nc2nc3ccccc3s2)nnc2c1CCCN2c1ccc(-c2cnn(C[C@]34C[C@@]5(C)C[C@](C)(C[C@](CCC[N+](C)(CCC(=O)O)Cc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9A-P58 | 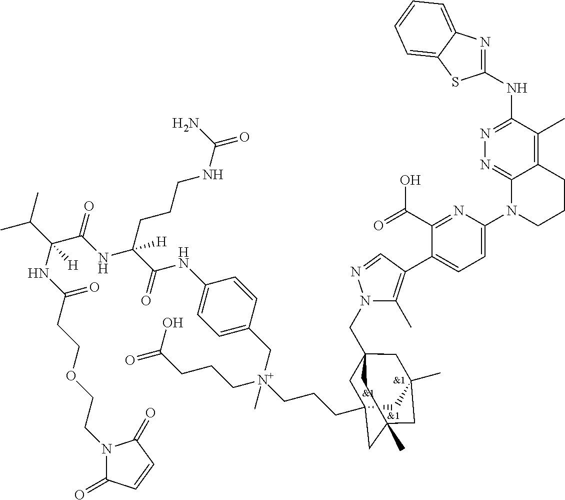 [SMILES: Cc1c(Nc2nc3ccccc3s2)nnc2c1CCCN2c1ccc(-c2cnn(C[C@]34C[C@@]5(C)C[C@](C)(C[C@](CCC[N+](C)(CCCC(=O)O)Cc6ccc(NC(=O)[C@H](CCCNC(N)=O)NC(=O)[C@@H](NC(=O)CCOCCN7C(=O)C=CC7=O)C(C)C)cc6)(C5)C3)C4)c2C)c(C(=O)O)n1] |
| L9A-P57 | 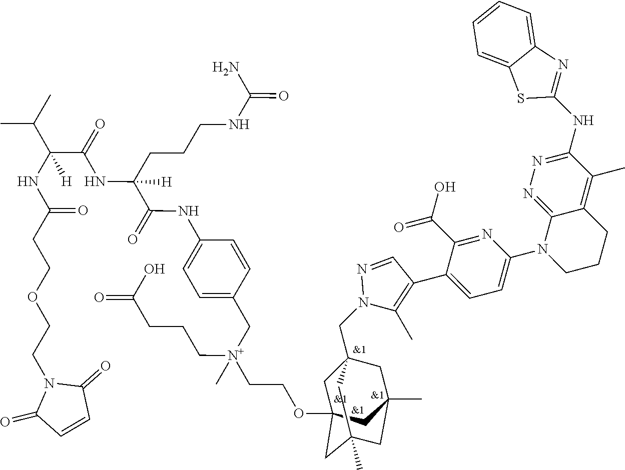 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+](C)(CCCC(=O)O)Cc3ccc(NC(=O)[C@H](CCCNC(N)=O)NC(=O)[C@@H](NC(=O)CCOCCN6C(=O)C=CC6=O)C(C)C)cc3)(C5)C4)c2C)c(C(=O)O)n1] |
| L9A-P73 | 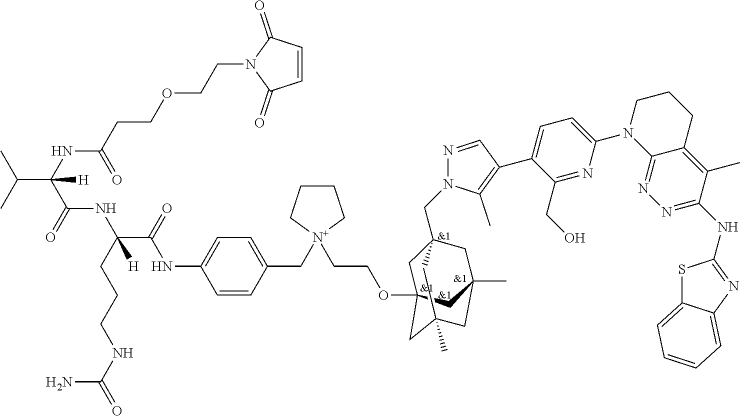 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(CO)n1] |
| L9A-P74 | 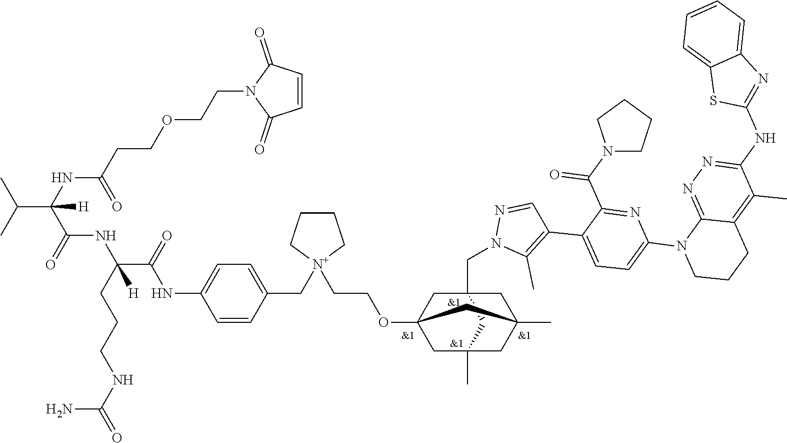 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(C(=O)N2CCCC2)n1] |
| L9A-P75 | 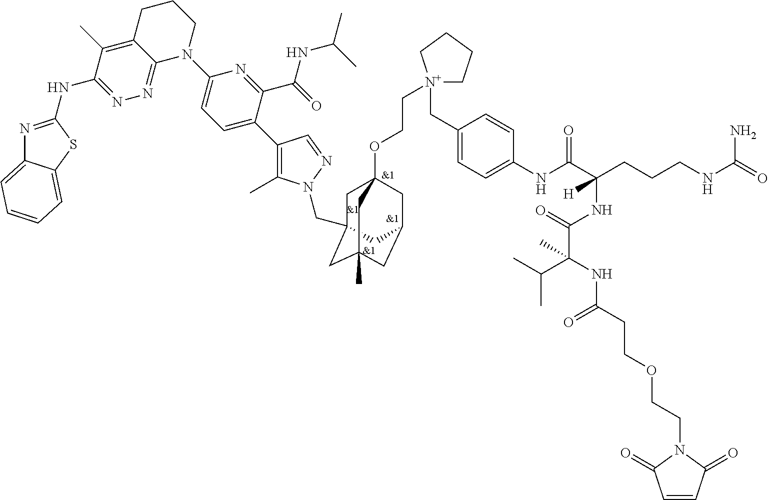 [SMILES: Cc1c(Nc2nc3ccccc3s2)nnc2c1CCCN2c1ccc(-c2cnn(C[C@]34C[C@@H]5C[C@](C)(C3)C[C@@](OCC[N+]3(Cc6ccc(NC(=O)[C@H](CCCNC(N)=O)NC(=O)[C@@](C)(NC(=O)CCOCCN7C(=O)C=CC7=O)C(C)C)cc6)CCCC3)(C5)C4)c2C)c(C(=O)NC(C)C)n1] |
| L9A-P76 | 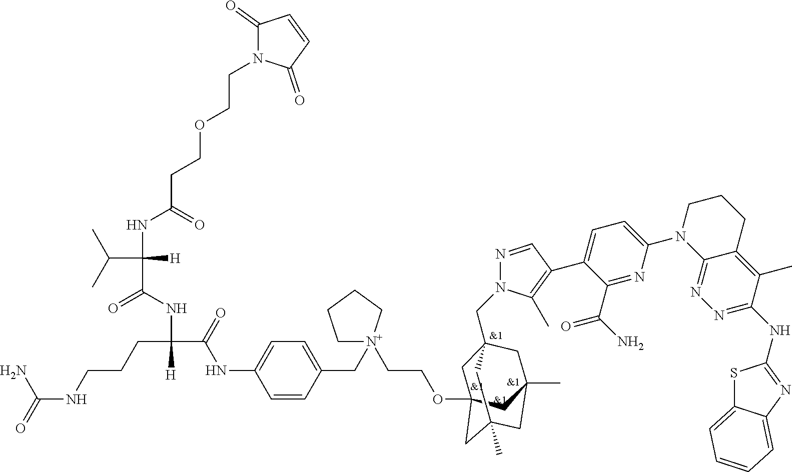 [SMILES: Cc1c(Nc2nc3ccccc3s2)nnc2c1CCCN2c1ccc(-c2cnn(C[C@]34C[C@]5(C)C[C@](C)(C3)C[C@@](OCC[N+]3(Cc6ccc(NC(=O)[C@H](CCCNC(N)=O)NC(=O)[C@@H](NC(=O)CCOCCN7C(=O)C=CC7=O)C(C)C)cc6)CCCC3)(C5)C4)c2C)c(C(N)=O)n1] |
| L10A-P1 | 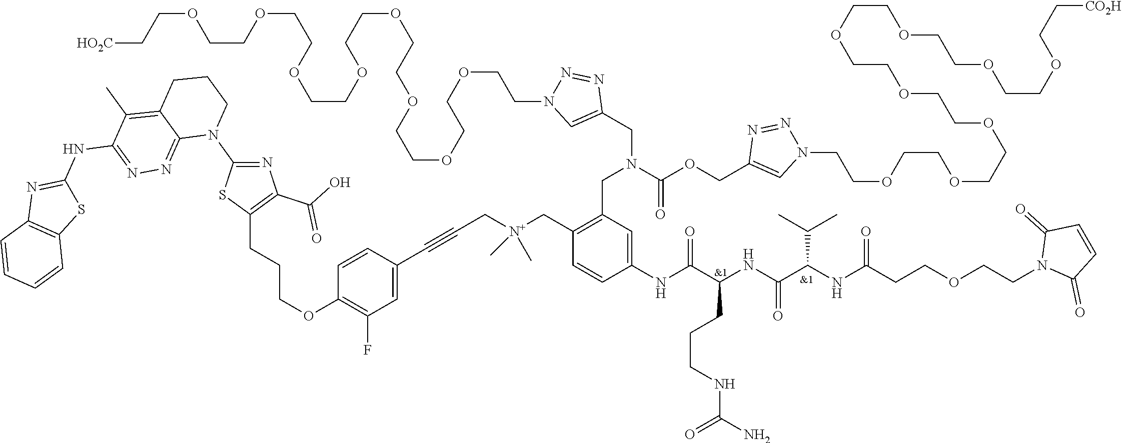 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(Cc3cn(CCOCCOCCOCCOCCOCCOCCOCCOCCC(=O)O)nn3)C(=O)OCc3cn(CCOCCOCCOCCOCCOCCOCCOCCOCCC(=O)O)nn3)cc2F)s1] |
| L10A-P2 | 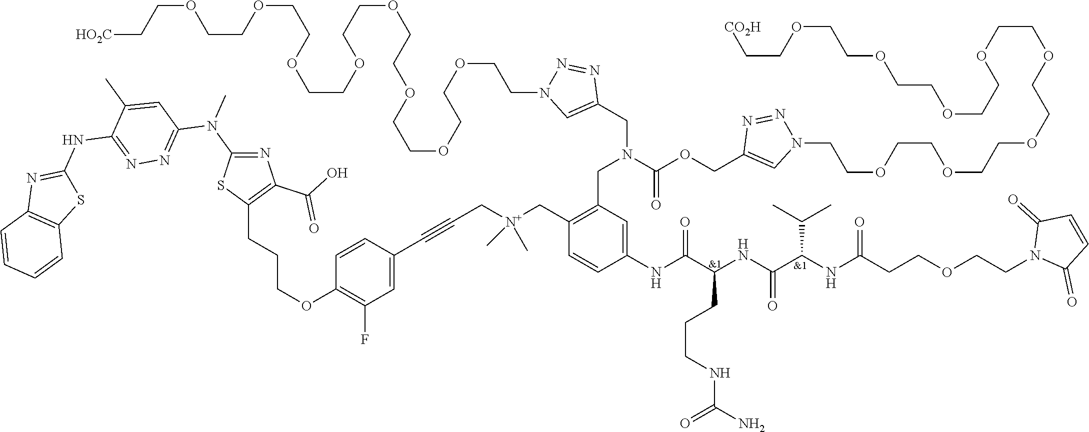 [SMILES: Cc1cc(N(C)c2nc(C(=O)O)c(CCCOc3ccc(C#CC[N+](C)(C)Cc4ccc(NC(=O)[C@H](CCCNC(N)=O)NC(=O)[C@@H](NC(=O)CCOCCN5C(=O)C=CC5=O)C(C)C)cc4CN(Cc4cn(CCOCCOCCOCCOCCOCCOCCOCCOCCC(=O)O)nn4)C(=O)OCc4cn(CCOCCOCCOCCOCCOCCOCCOCCOCCC(=O)O)nn4)cc3F)s2)nnc1Nc1nc2ccccc2s1] |
| L10C-P3 | 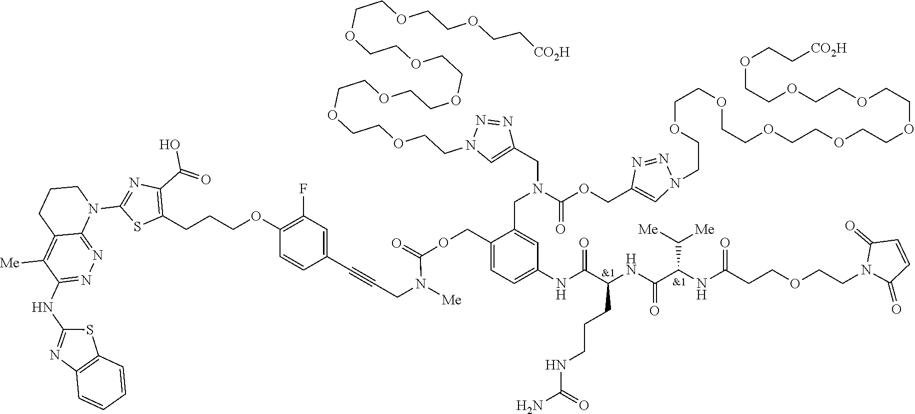 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3CN(Cc3cn(CCOCCOCCOCCOCCOCCOCCOCCOCCC(=O)O)nn3)C(=O)OCc3cn(CCOCCOCCOCCOCCOCCOCCOCCOCCC(=O)O)nn3)cc2F)s1] |
| L11A-P1 | 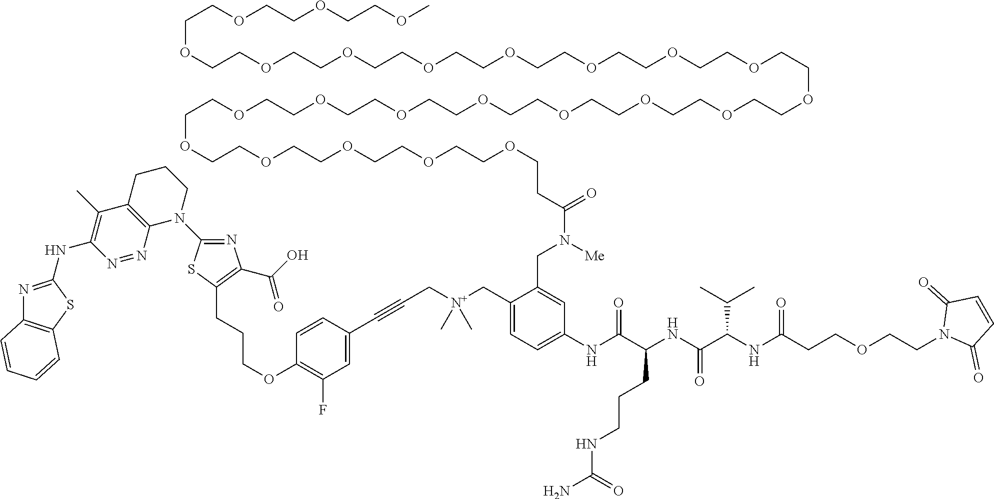 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1] |
| L11A-P21 | 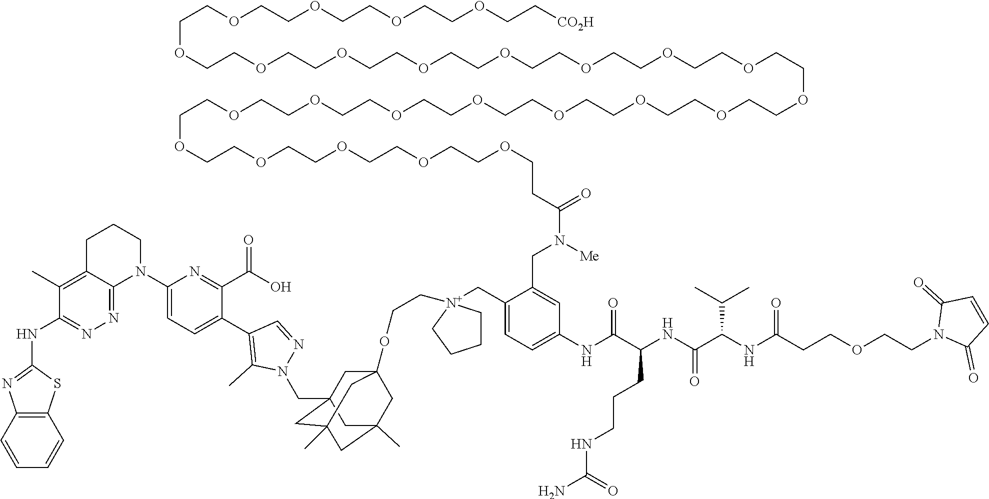 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)CCCC3)(C5)C4)c2C)c(C(=O)O)n1] |
| L11C-P25 | 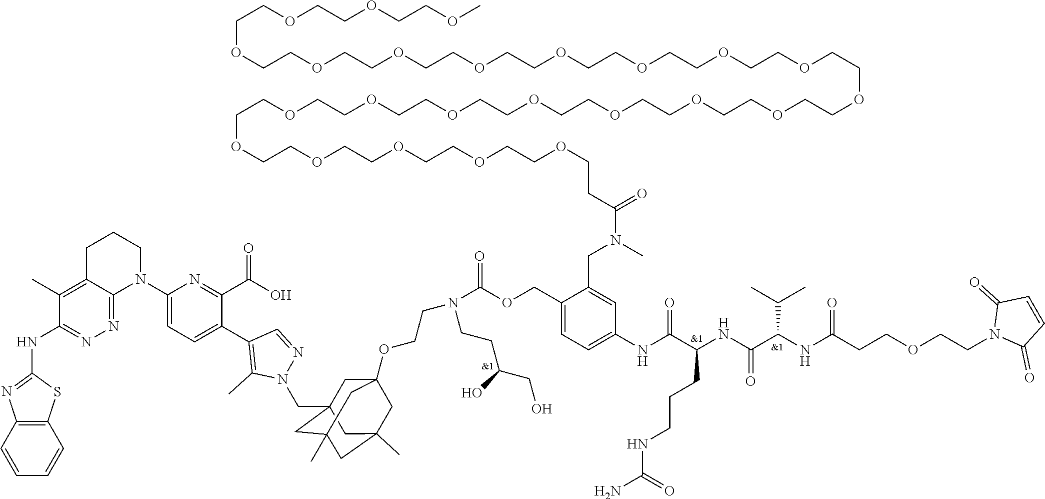 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO] |
| L11A-P27 | 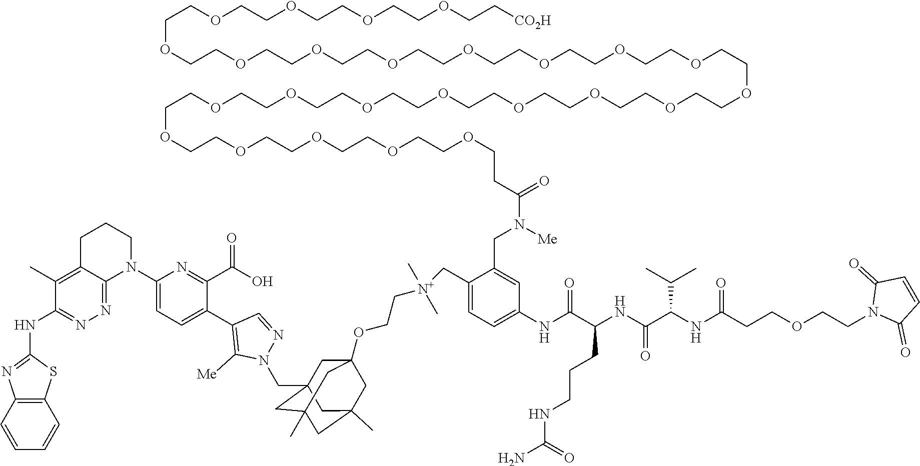 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1] |
| L11C-P19 | 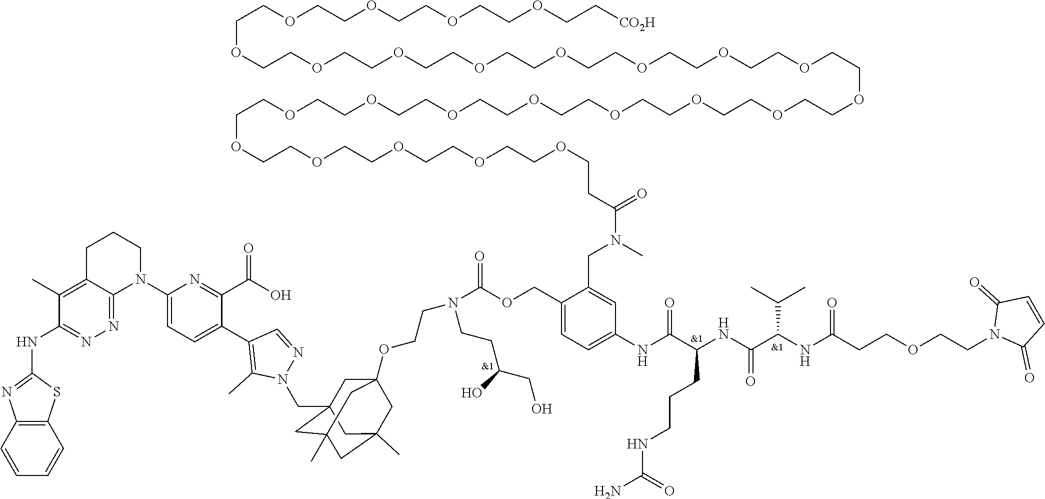 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1] |
| L13A-P2 | 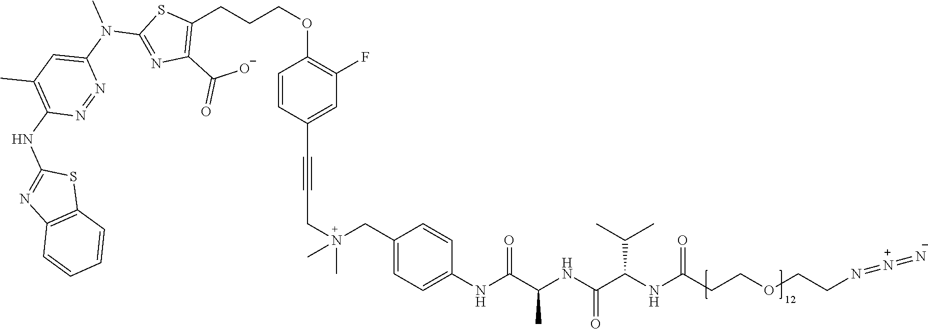 |
| L19C-P7 | 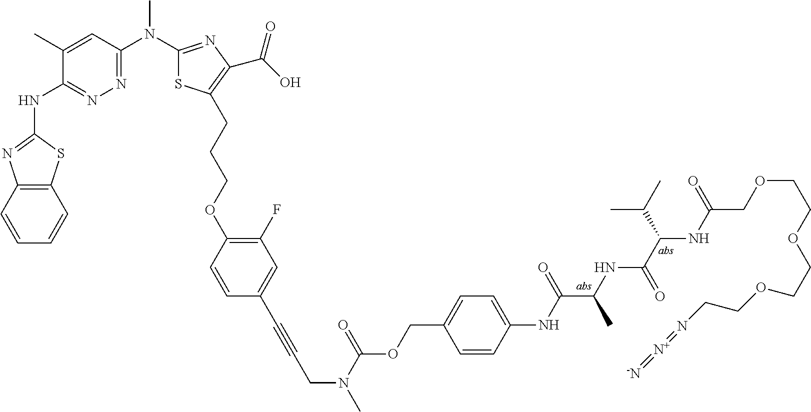 |
| L21A-P2 | 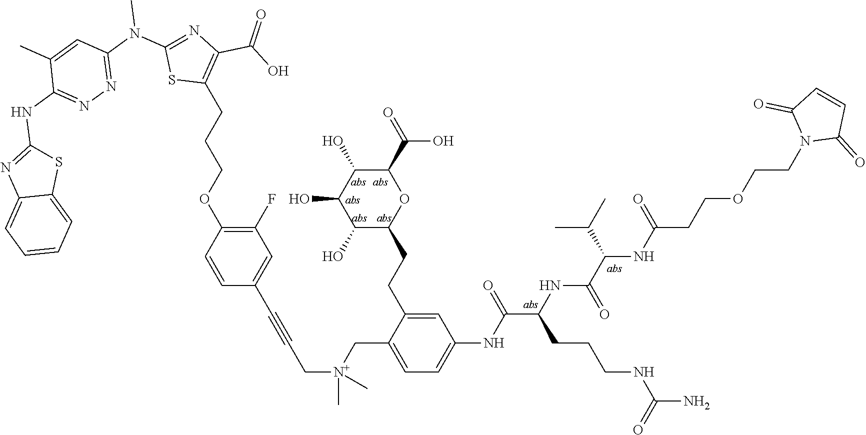 [SMILES: Cc1cc(N(C)c2nc(C(=O)O)c(CCCOc3ccc(C#CC[N+](C)(C)Cc4ccc(NC(=O)[C@H](CCCNC(N)=O)NC(=O)[C@@H](NC(=O)CCOCCN5C(=O)C=CC5=O)C(C)C)cc4CC[C@@H]4O[C@H](C(=O)O)[C@@H](O)[C@H](O)[C@H]4O)cc3F)s2)nnc1Nc1nc2ccccc2s1] |
| L23C-P7 | 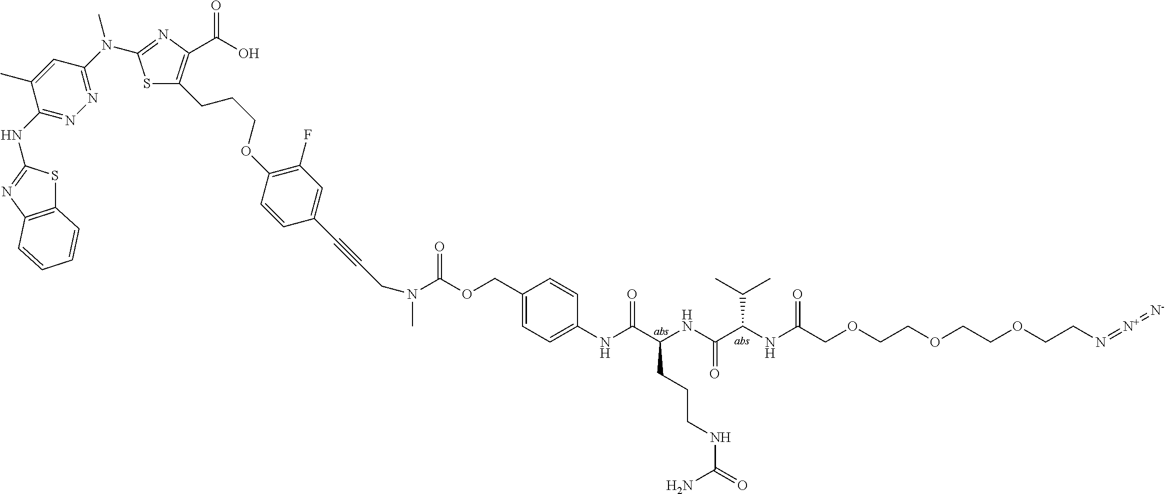 [SMILES: Cc1cc(N(C)c2nc(C(=O)O)c(CCCOc3ccc(C#CCN(C)C(=O)OCc4ccc(NC(=O)[C@H](CCCNC(N)=O)NC(=O)[C@@H](NC(=O)COCCOCCOCCN=[N+]=[N-])C(C)C)cc4)cc3F)s2)nnc1Nc1nc2ccccc2s1] |
| L27C-P3 | 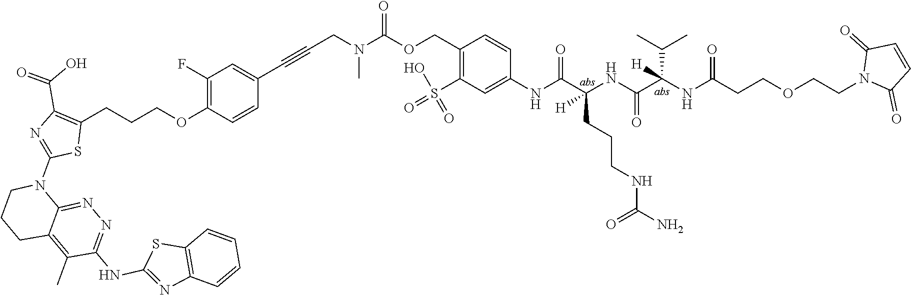 [SMILES: Cc1c(Nc2nc3ccccc3s2)nnc2c1CCCN2c1nc(C(=O)O)c(CCCOc2ccc(C#CCN(C)C(=O)OCc3ccc(NC(=O)[C@H](CCCNC(N)=O)NC(=O)[C@@H](NC(=O)CCOCCN4C(=O)C=CC4=O)C(C)C)cc3S(=O)(=O)O)cc2F)s1] |
| L27A-P1 | 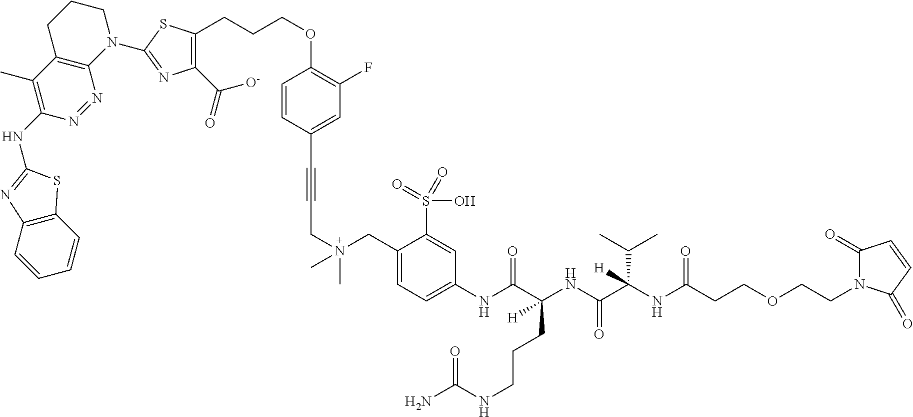 |
| L30A-P1 | 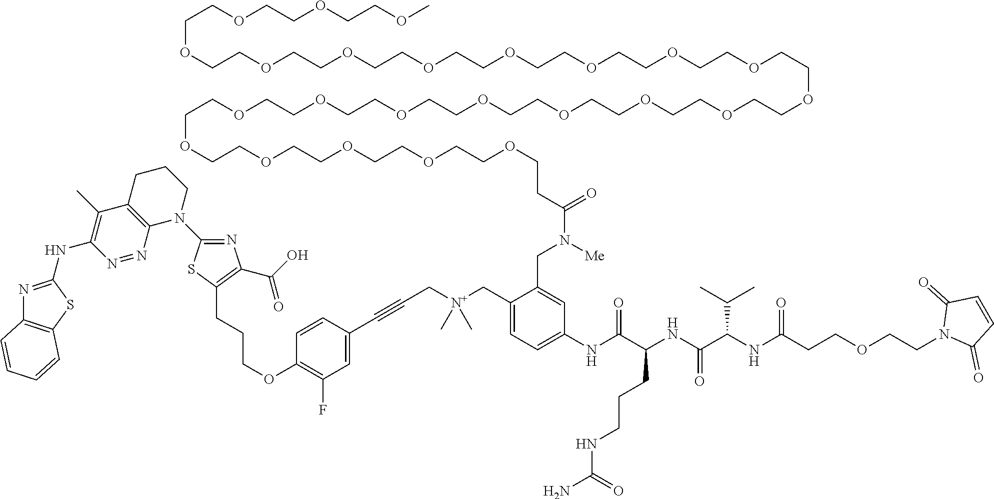 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1] |
| L30C-P19 | 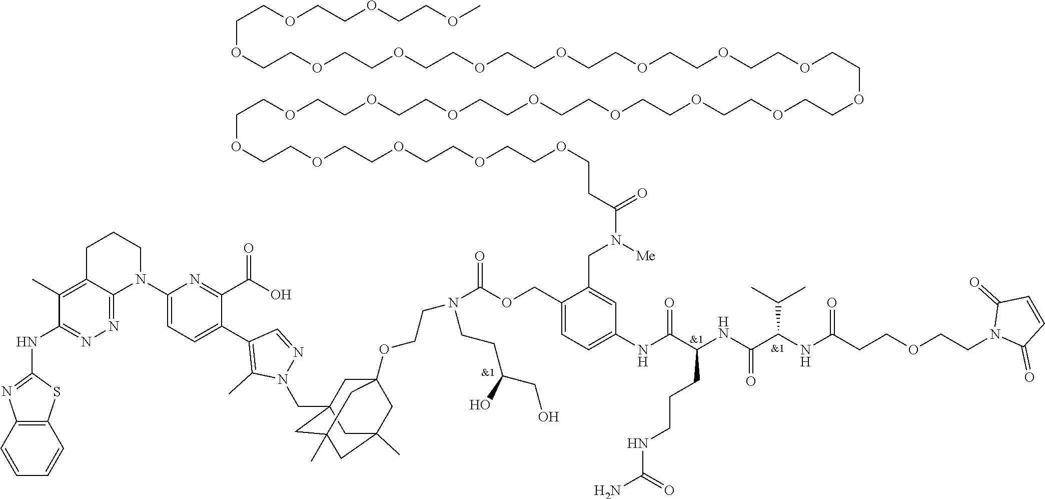 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO] |
| L30A-P21 | 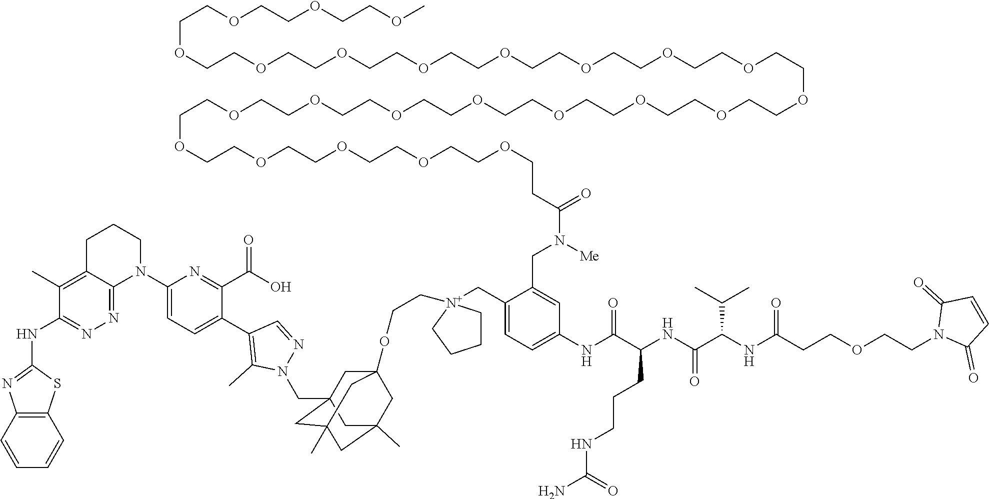 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1] |
| L30C-P25 | 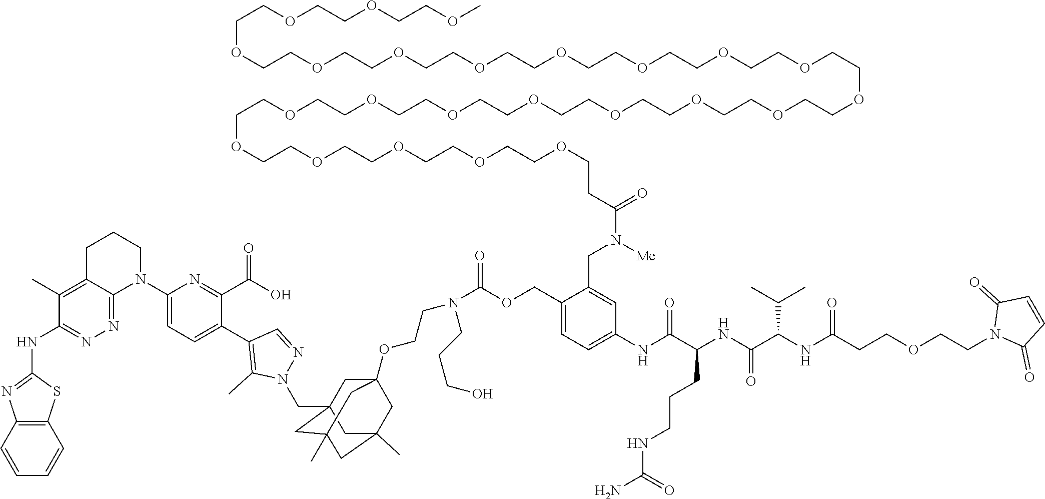 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCCO)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L30A-P27 | 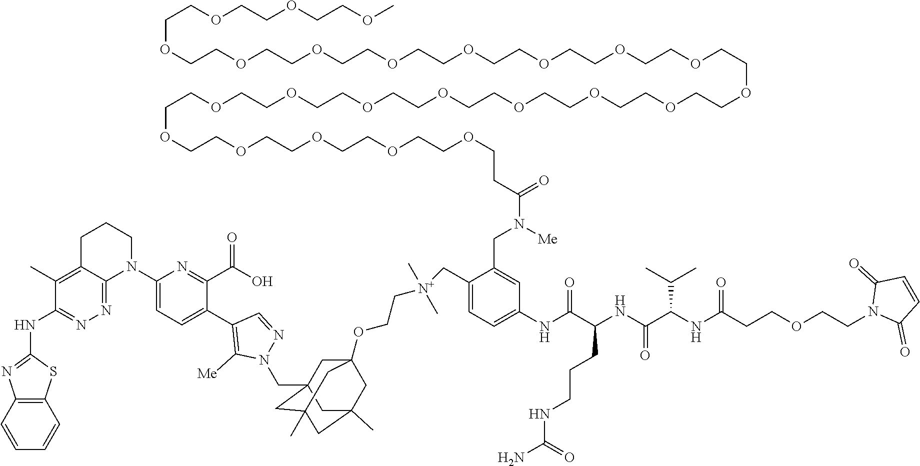 [SMILES: COCCOCCOCCOCCOCCOCCOCCOCCOCCOCCOCCOCCOCCOCCOCCOCCOCCOCCOCCOCCOCCOCCOCCOCCC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L35A-P1 | 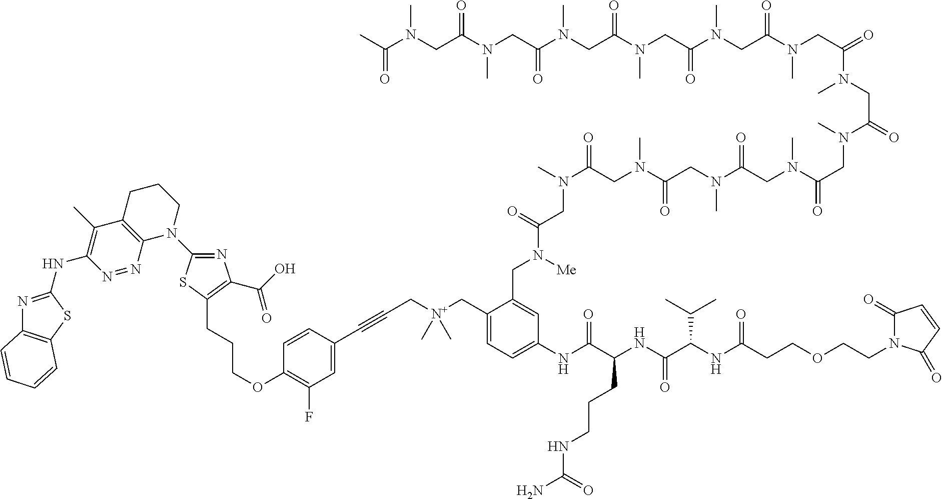 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1] |
| L35C-P19 | 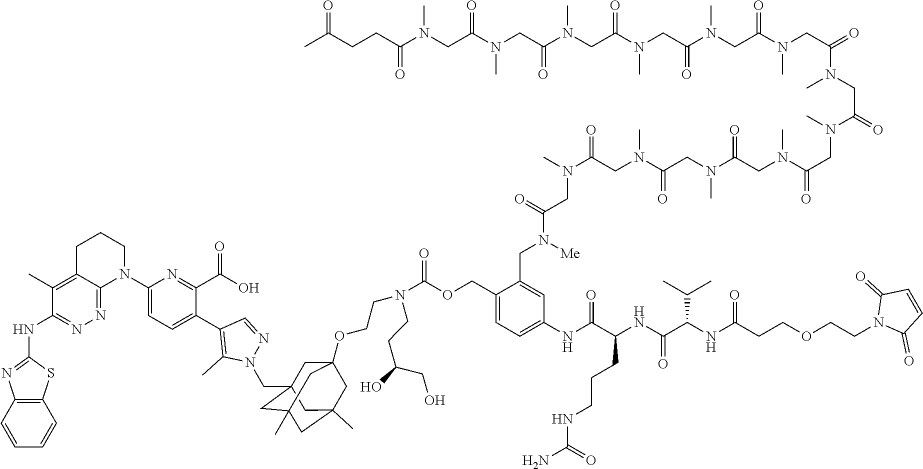 [SMILES: CC(=O)CCC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO] |
| L35A-P21 | 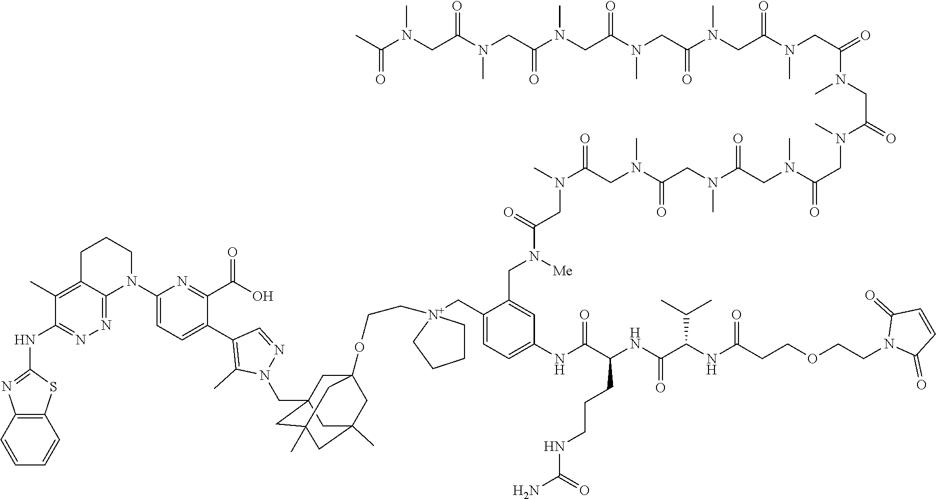 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1] |
| L35C-P25 | 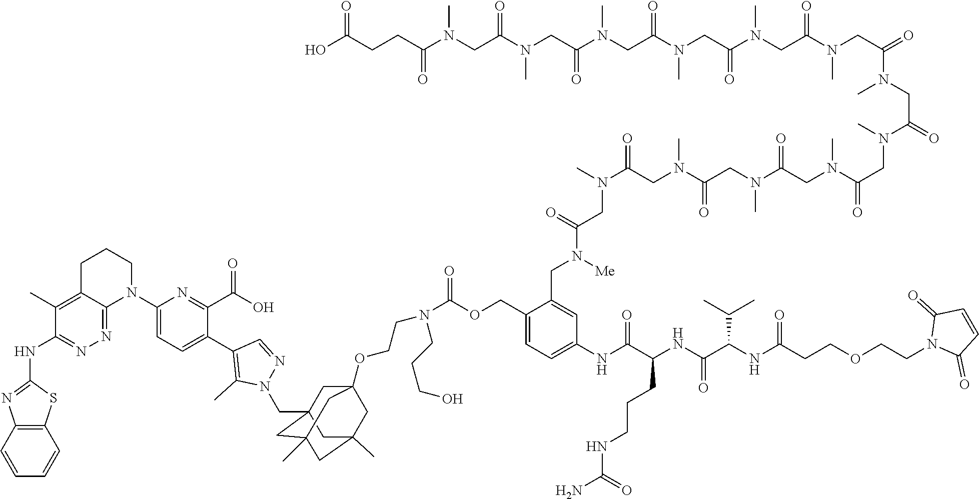 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L35A-P27 | 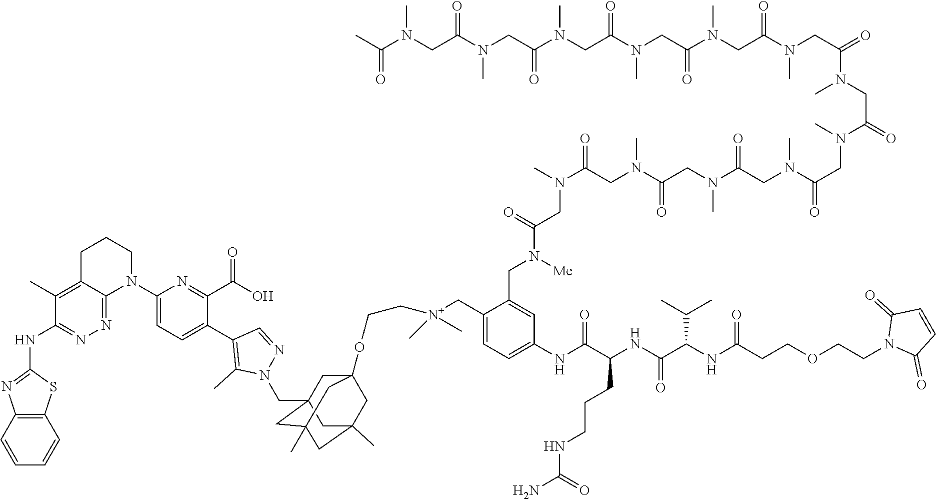 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L36A-P1 | 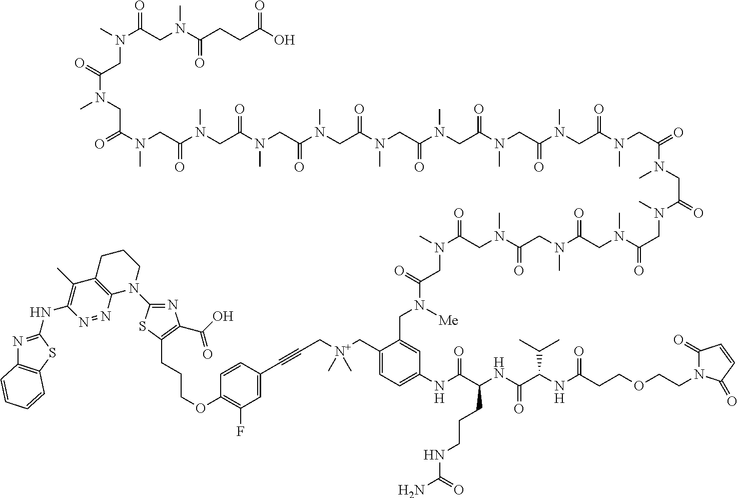 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)cc2F)s1] |
| L36C-P19 | 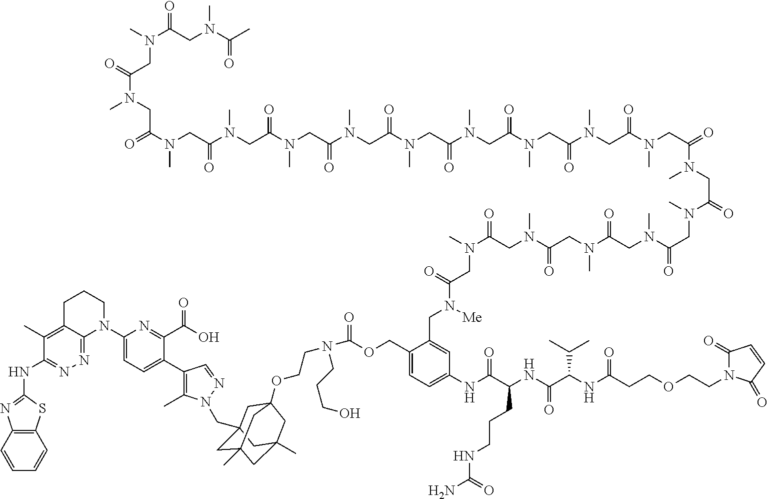 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCCO)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L36A-P21 | 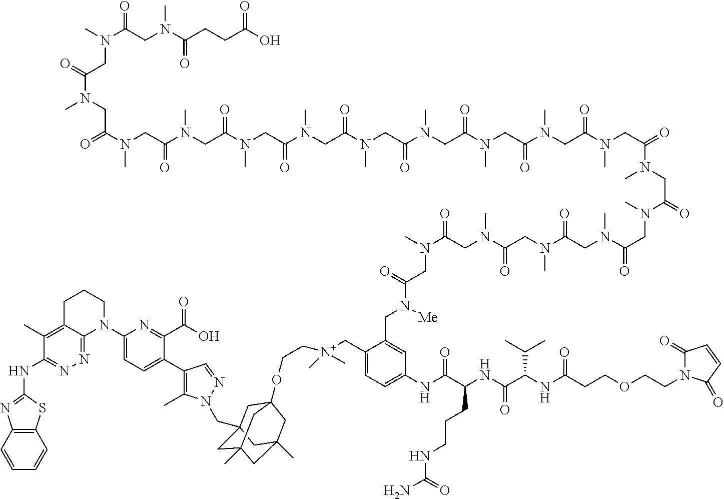 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L36C-P25 | 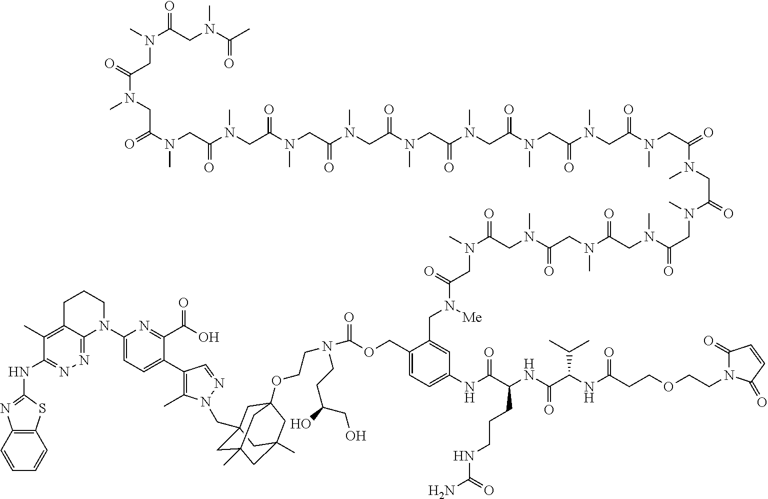 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO] |
| L36A-P27 | 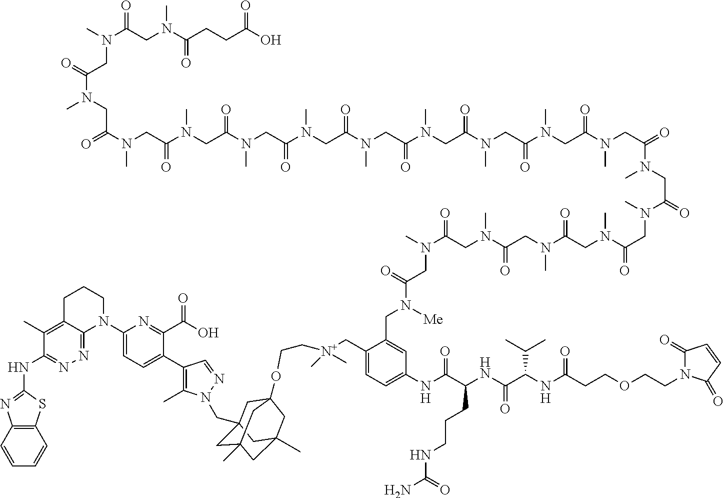 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L37A-P1 | 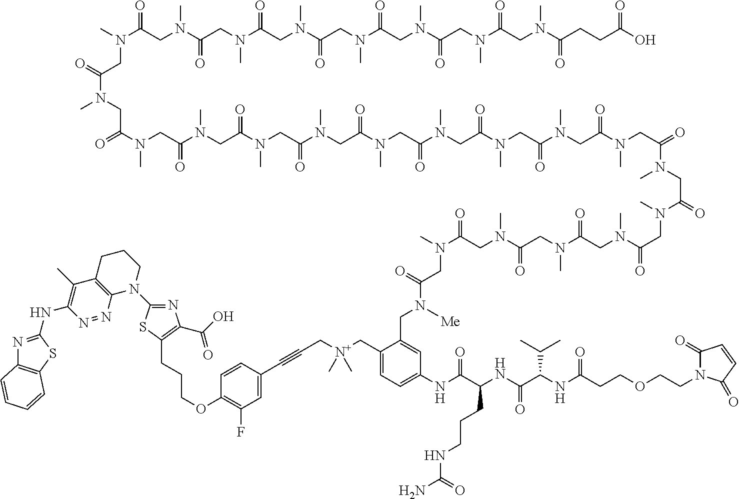 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)cc2F)s1] |
| L37C-P19 | 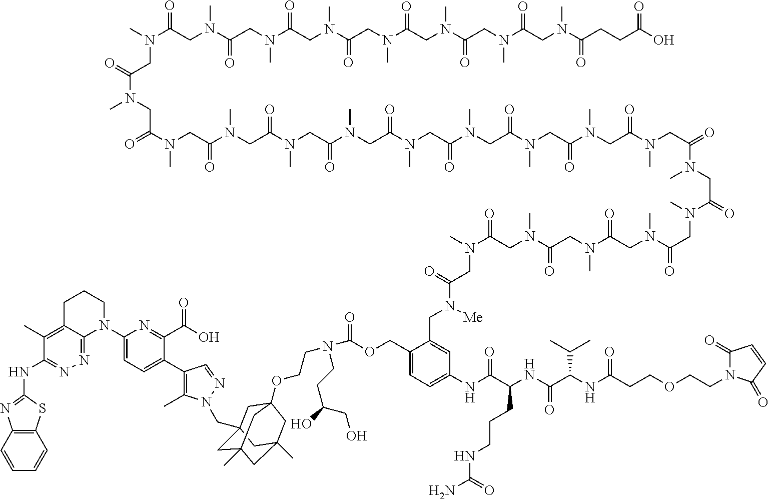 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L37A-P21 | 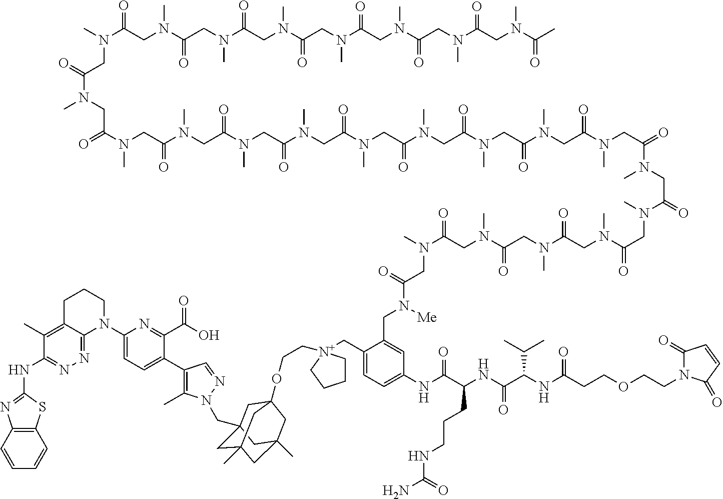 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1] |
| L37C-P25 | 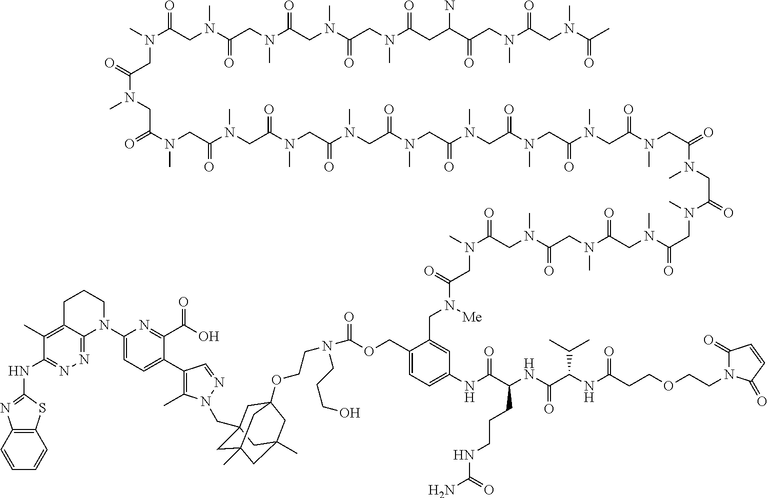 [SMILES: CC(=O)N(C)CC(=O)N(C)CC(=O)C(N)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCCO)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L37A-P27 | 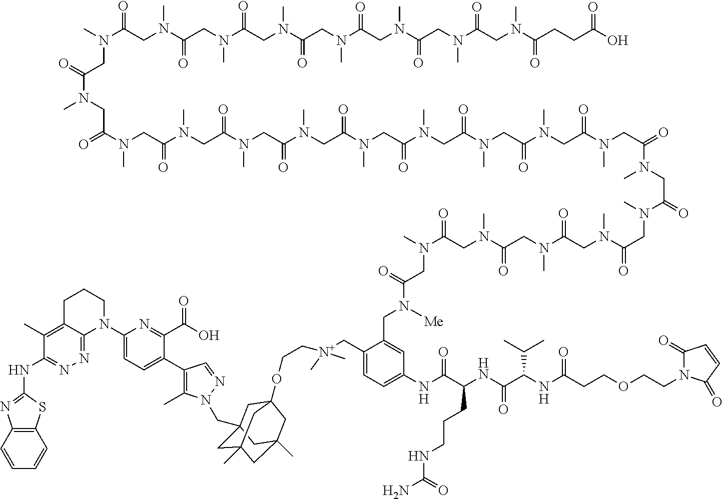 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O)(C5)C4)c2C)c(C(=O)O)n1] |
| L38A-P1 | 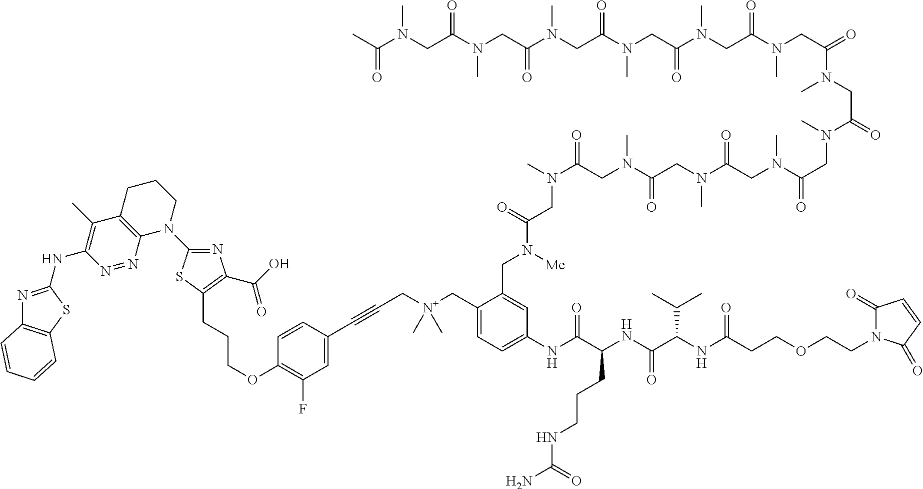 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1] |
| L38C-P19 | 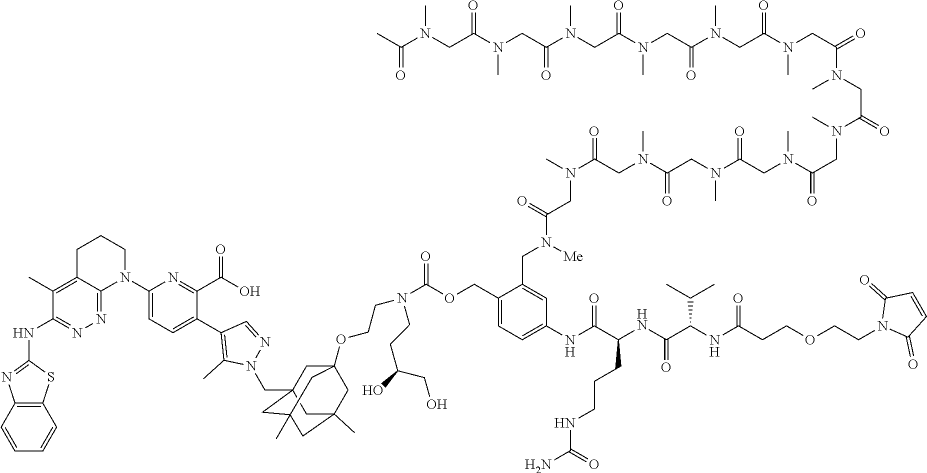 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO] |
| L38A-P21 | 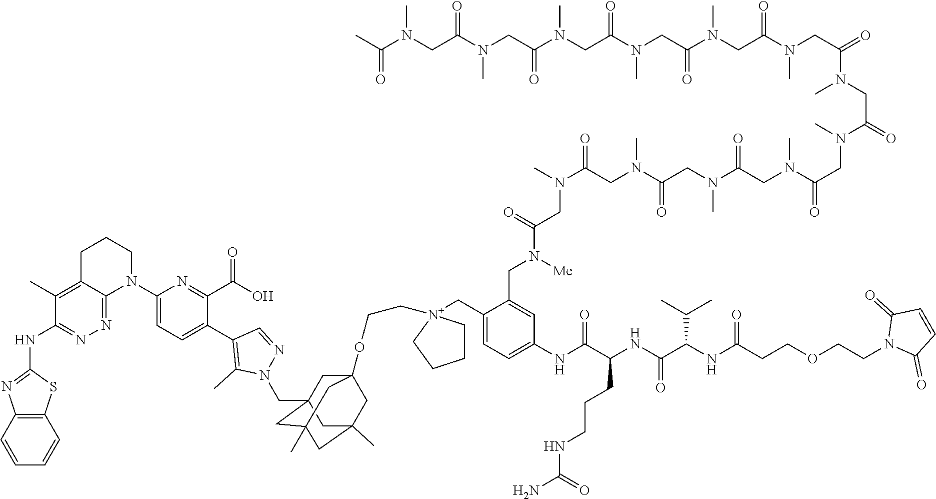 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1] |
| L38C-P25 | 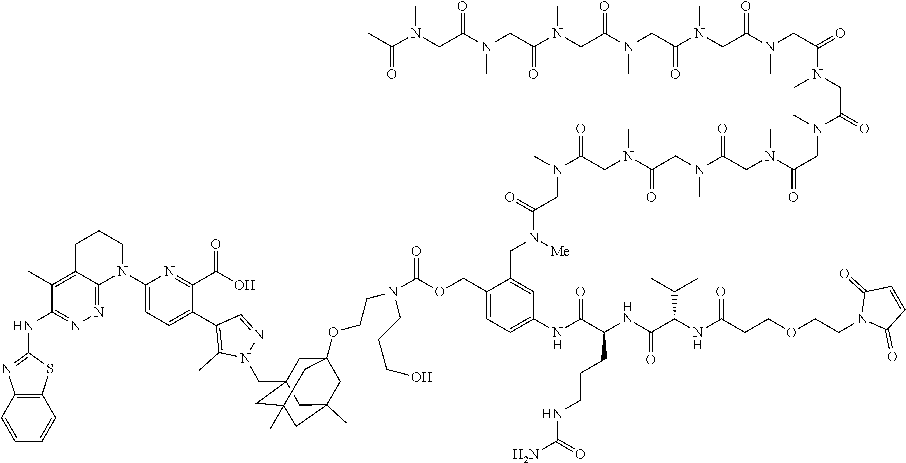 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCCO)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L38A-P27 | 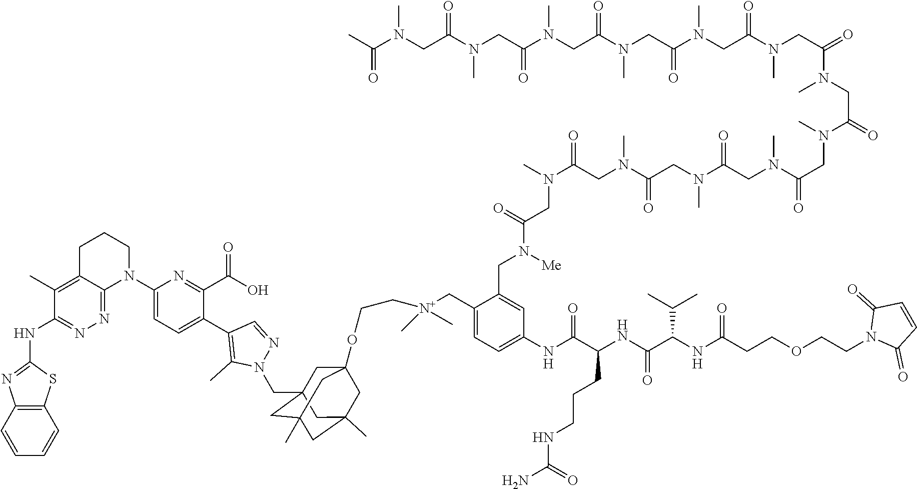 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L39A-P1 | 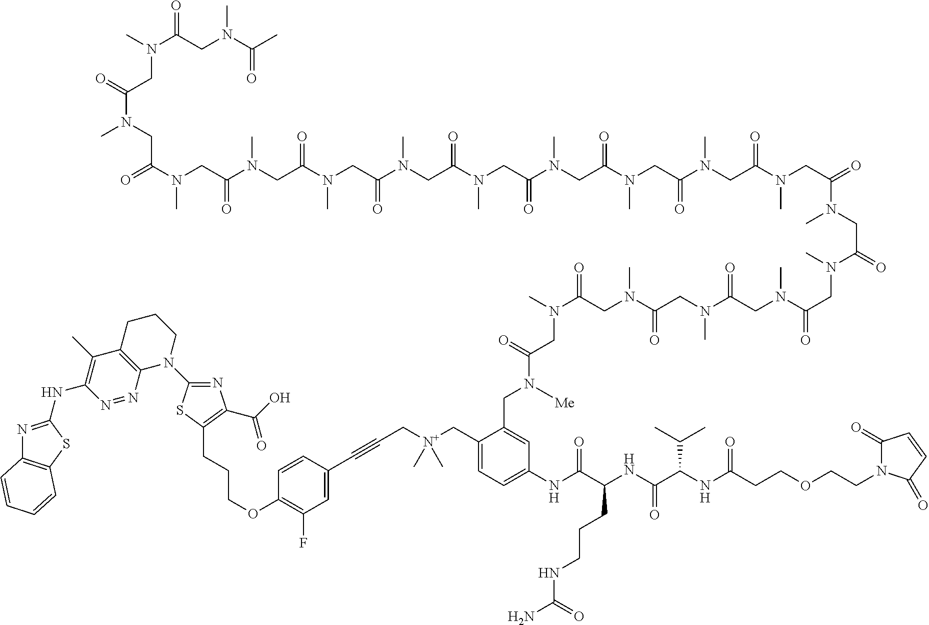 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1] |
| L39C-P19 | 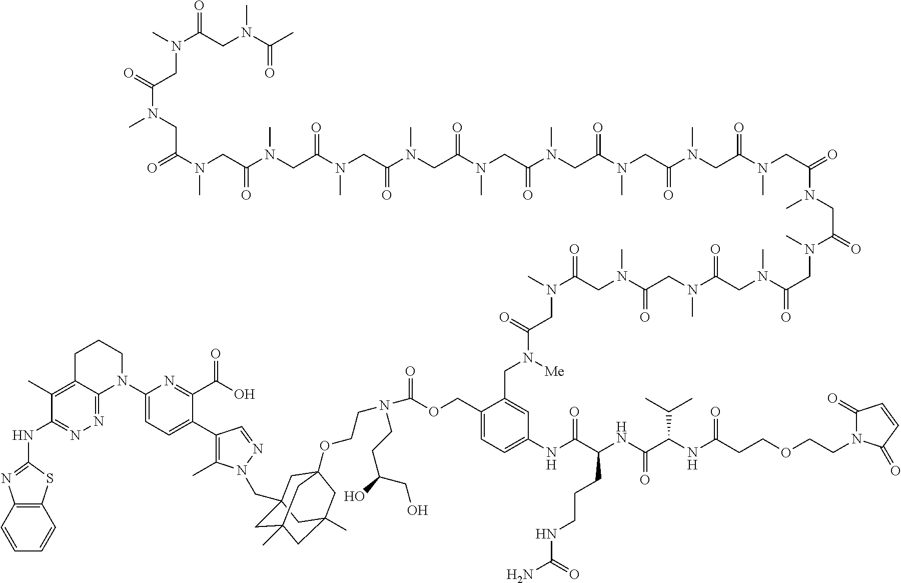 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO] |
| L39A-P21 | 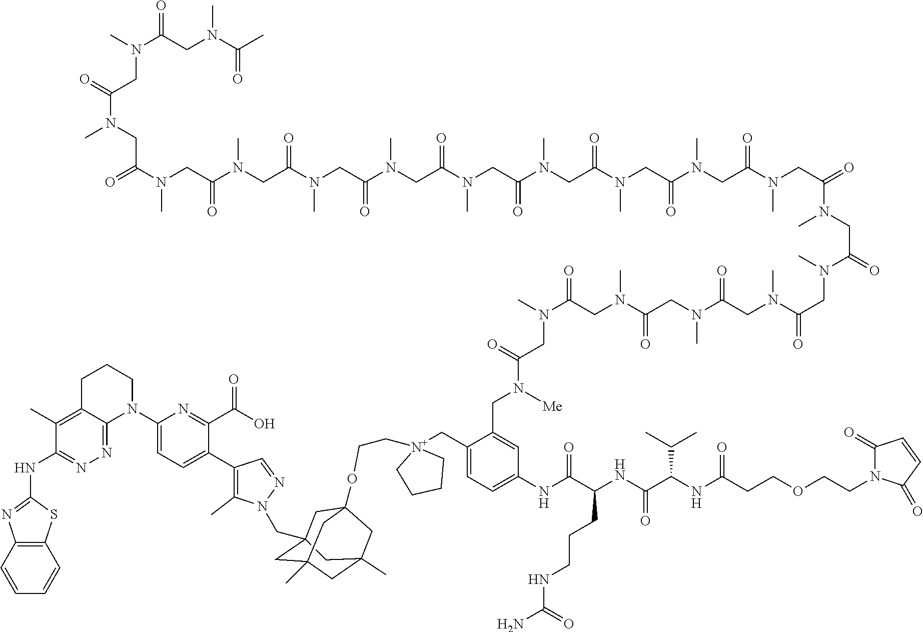 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1] |
| L39C-P25 | 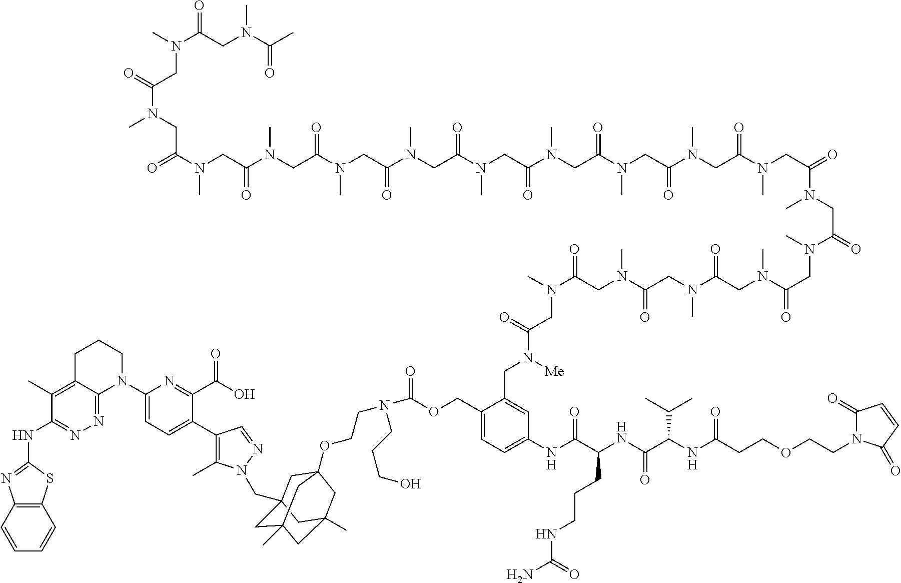 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCCO)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L39A-P27 | 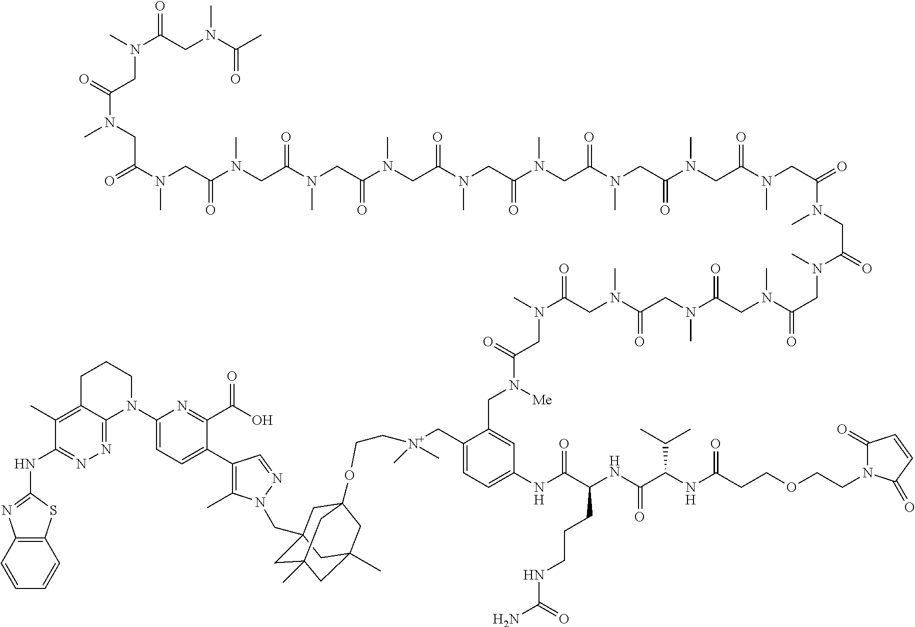 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L40A-P1 | 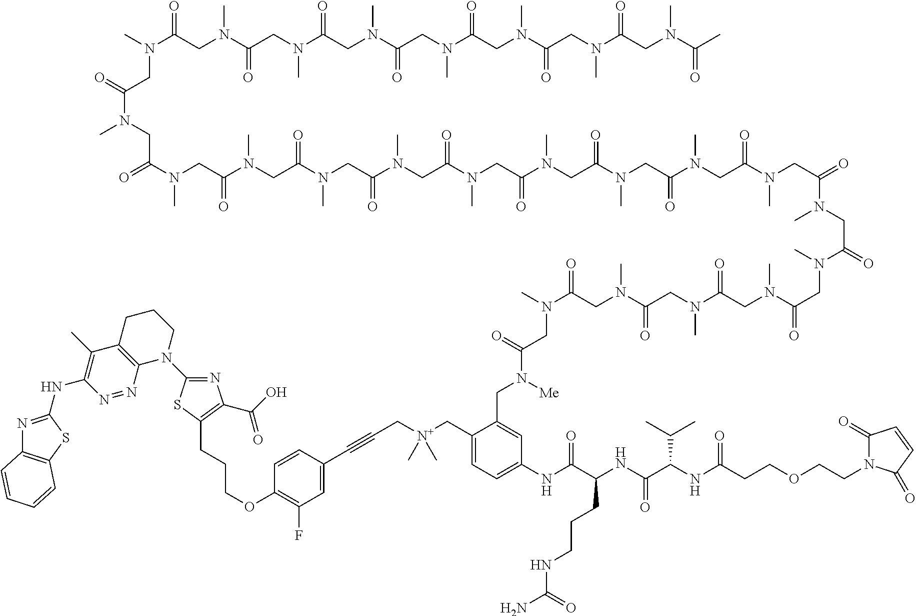 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1] |
| L40C-P19 | 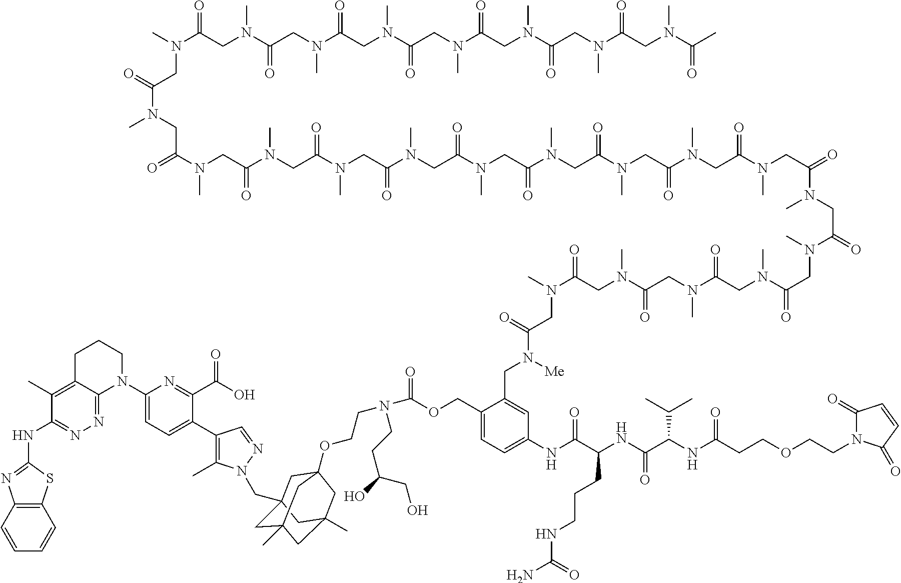 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO] |
| L40A-P21 | 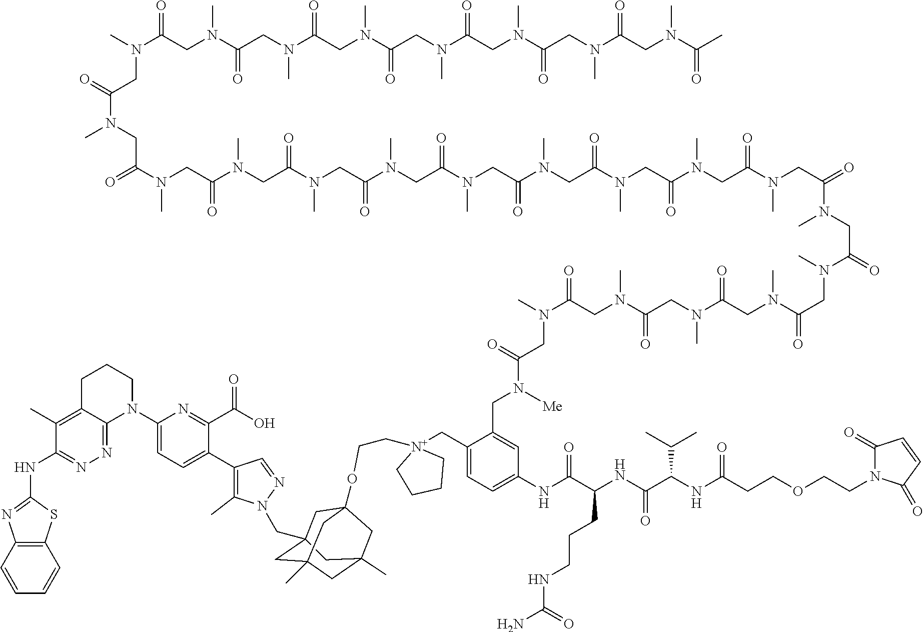 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1] |
| L40C-P25 | 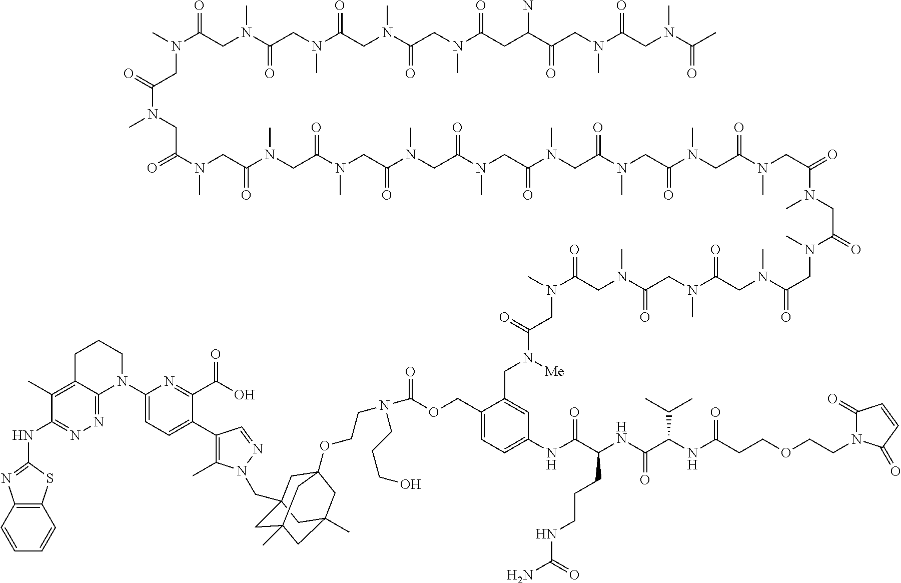 [SMILES: CC(=O)N(C)CC(=O)N(C)CC(=O)C(N)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCCO)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L40A-P27 | 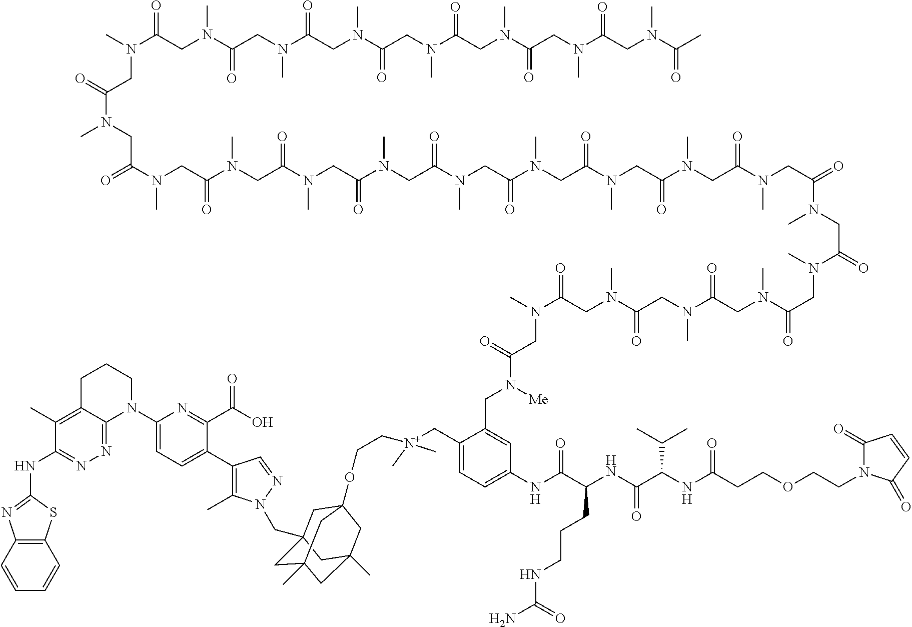 [SMILES: CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2] |
| L42A-P1 | 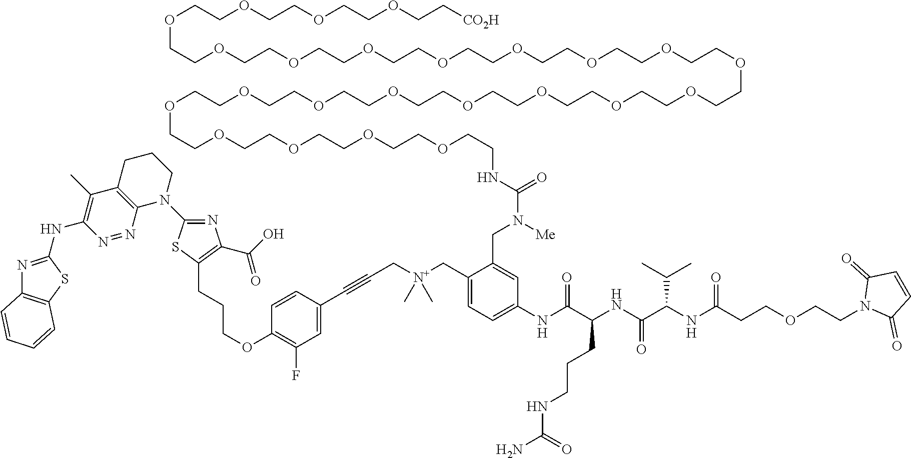 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)cc2F)s1] |
| L42C-P19 | 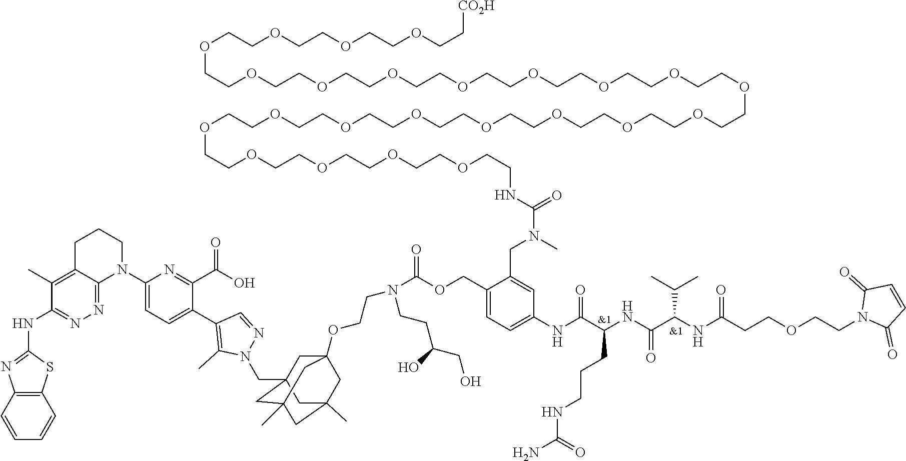 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1] |
| L42A-P21 | 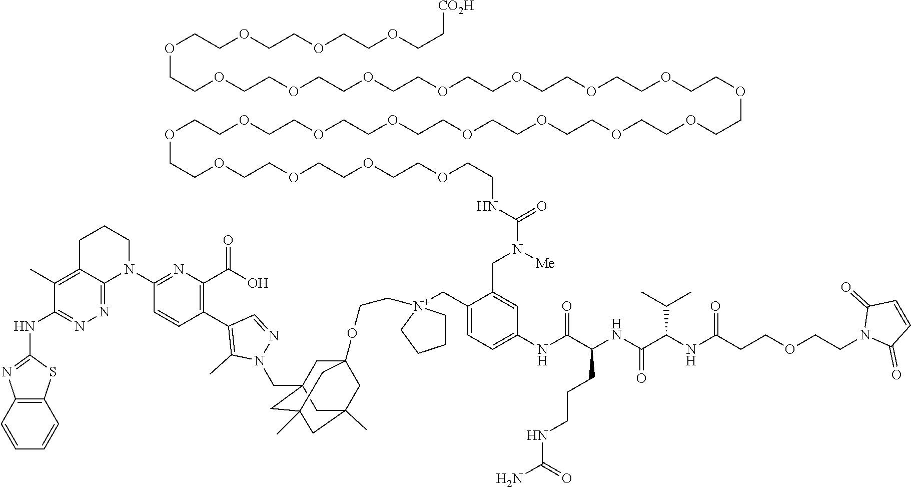 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CCCC3)(C5)C4)c2C)c(C(=O)O)n1] |
| L42C-P25 | 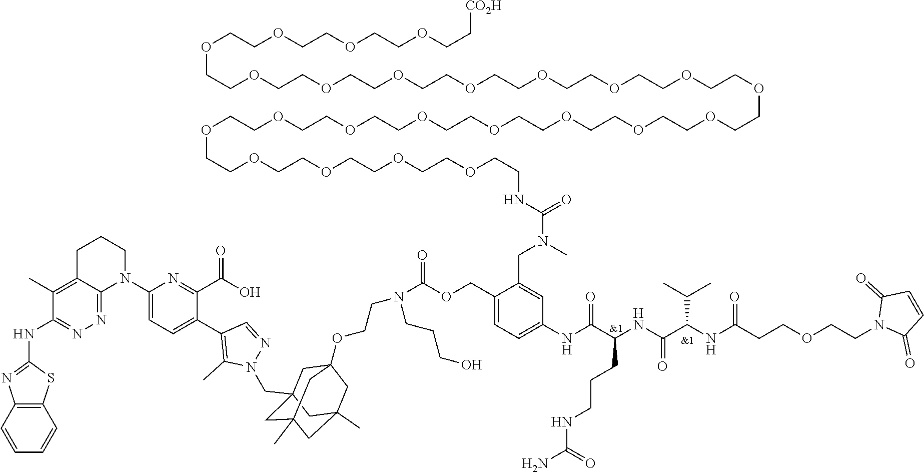 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1] |
| L42A-P27 | 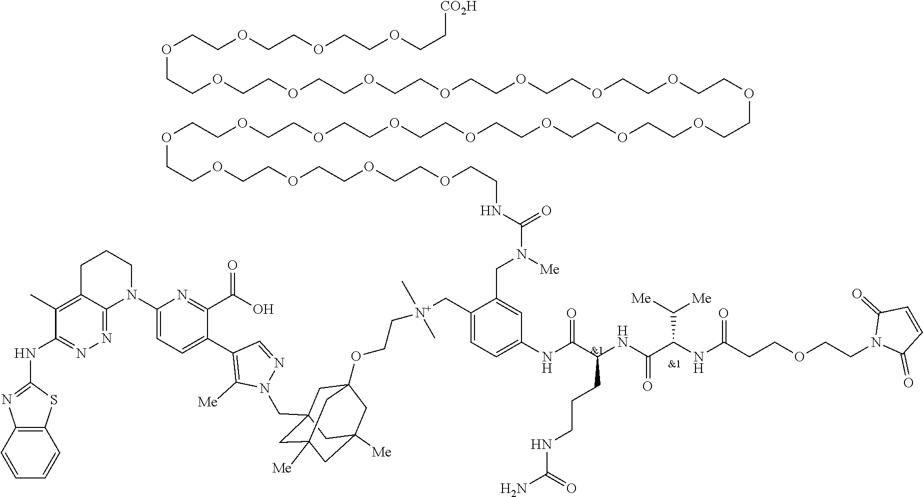 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)(C5)C4)c2C)c(C(=O)O)n1] |
| L67A-P1 | 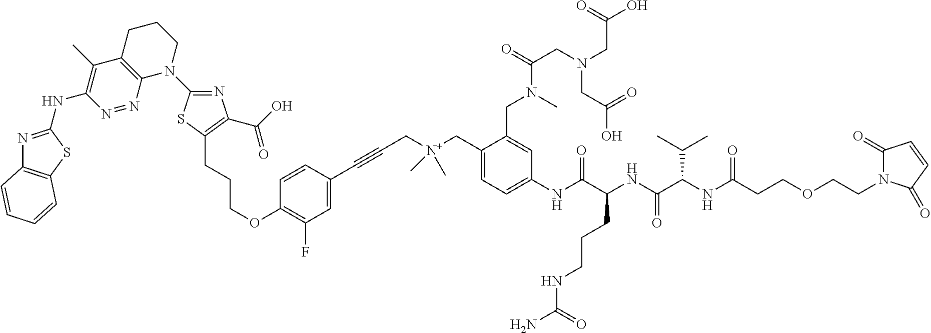 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)CN(CC(=O)O)CC(=O)O)cc2F)s1] |
| L67C-P19 | 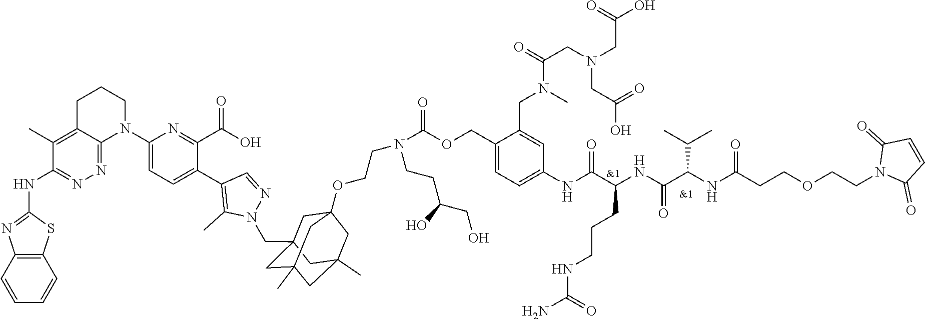 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CN(CC(=O)O)CC(=O)O)(C5)C4)c2C)c(C(=O)O)n1] |
| L67A-P21 | 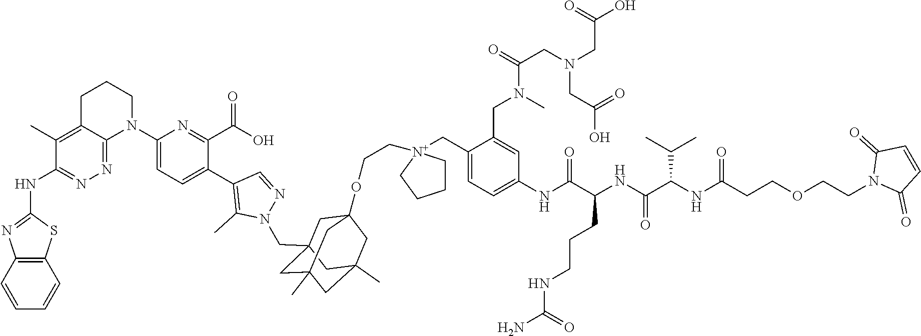 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)CN(CC(=O)O)CC(=O)O)CCCC3)(C5)C4)c2C)c(C(=O)O)n1] |
| L67C-P25 | 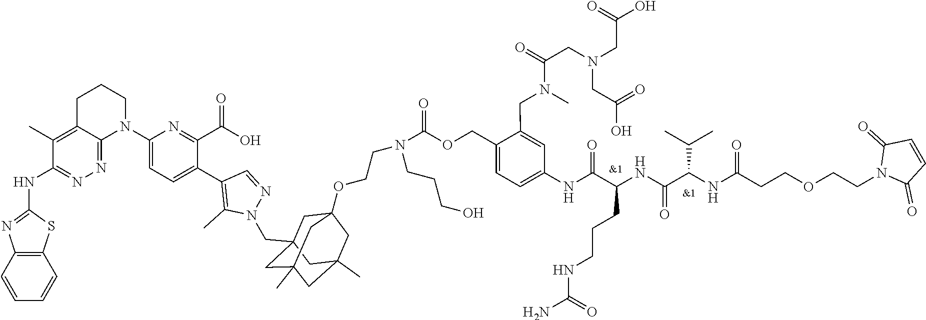 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)CN(CC(=O)O)CC(=O)O)(C5)C4)c2C)c(C(=O)O)n1] |
| L67A-P27 | 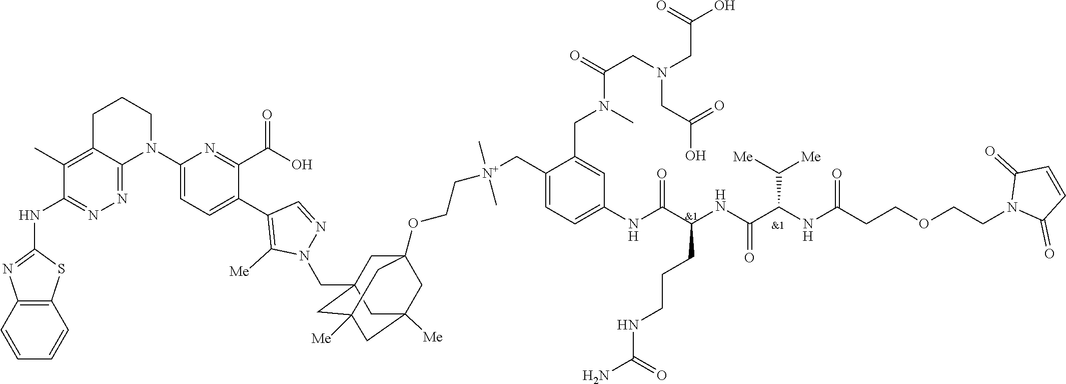 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)CN(CC(=O)O)CC(=O)O)(C5)C4)c2C)c(C(=O)O)n1] |
| L100A-P1 | 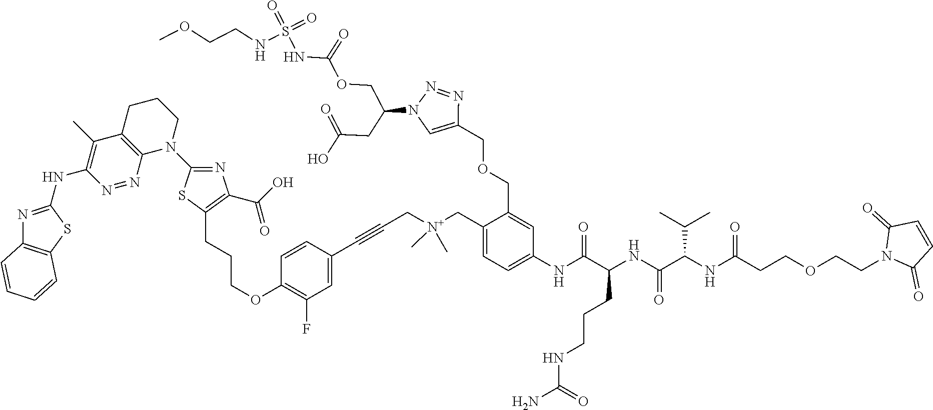 [SMILES: COCCNS(=O)(=O)NC(=O)OC[C@H](CC(=O)O)n1cc(COCc2cc(NC(=O)[C@H](CCCNC(N)=O)NC(=O)[C@@H](NC(=O)CCOCCN3C(=O)C=CC3=O)C(C)C)ccc2C[N+](C)(C)CC#Cc2ccc(OCCCc3sc(N4CCCc5c4nnc(Nc4nc6ccccc6s4)c5C)nc3C(=O)O)c(F)c2)nn1] |
| L100C-P19 | 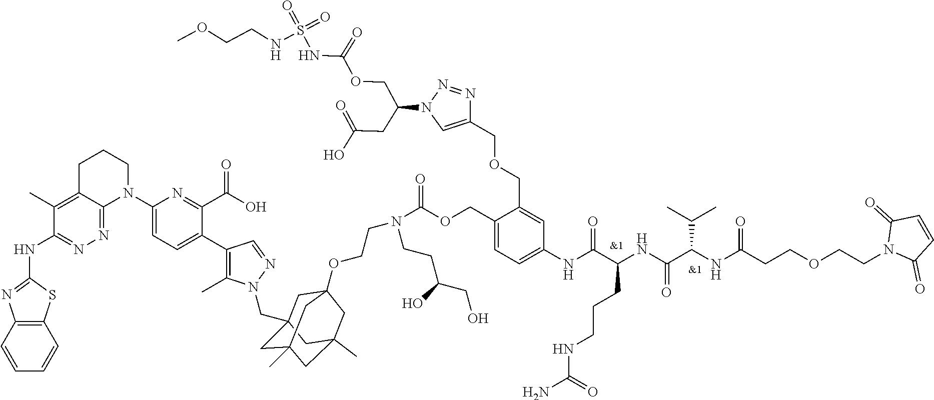 |
| L100A-P21 | 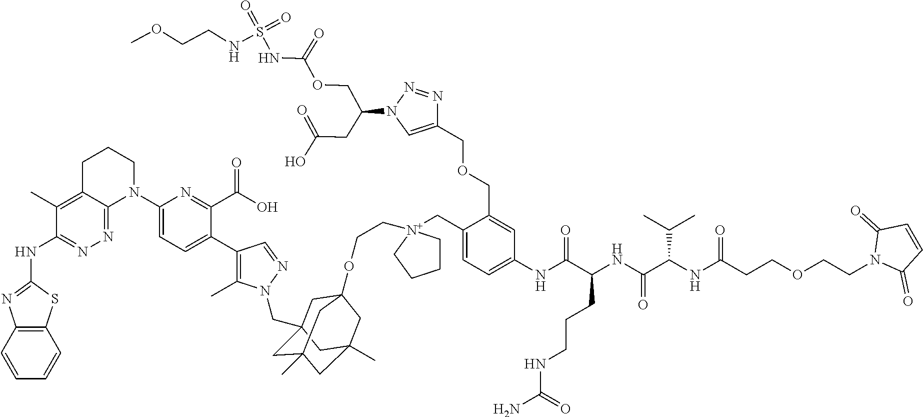 [SMILES: COCCNS(=O)(=O)NC(=O)OC[C@H](CC(=O)O)n1cc(COCc2cc(NC(=O)[C@H](CCCNC(N)=O)NC(=O)[C@@H](NC(=O)CCOCCN3C(=O)C=CC3=O)C(C)C)ccc2C[N+]2(CCOC34CC5(C)CC(C)(CC(Cn6ncc(-c7ccc(N8CCCc9c8nnc(Nc8nc%10ccccc%10s8)c9C)nc7C(=O)O)c6C)(C5)C3)C4)CCCC2)nn1] |
| L100C-P25 | 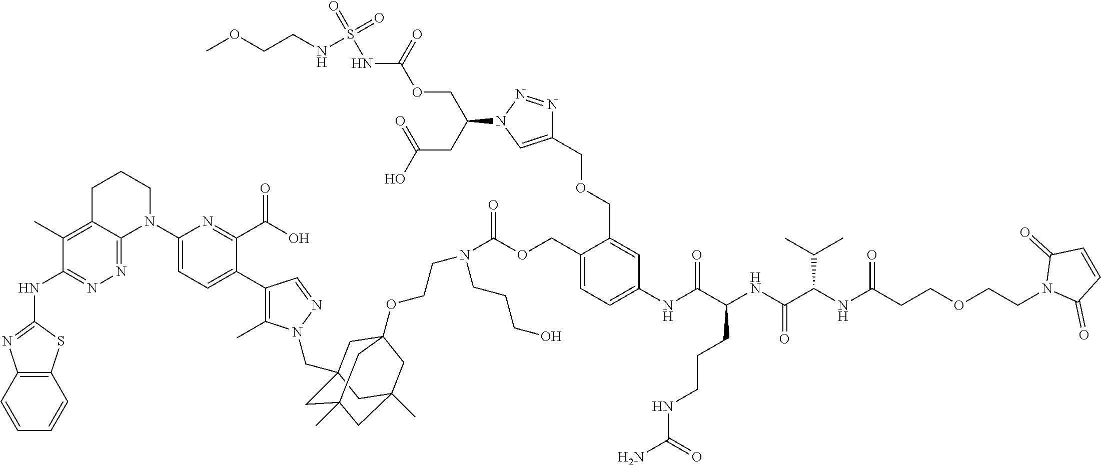 [SMILES: COCCNS(=O)(=O)NC(=O)OC[C@H](CC(=O)O)n1cc(COCc2cc(NC(=O)[C@H](CCCNC(N)=O)NC(=O)[C@@H](NC(=O)CCOCCN3C(=O)C=CC3=O)C(C)C)ccc2COC(=O)N(CCCO)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)nn1] |
| L100A-P27 | 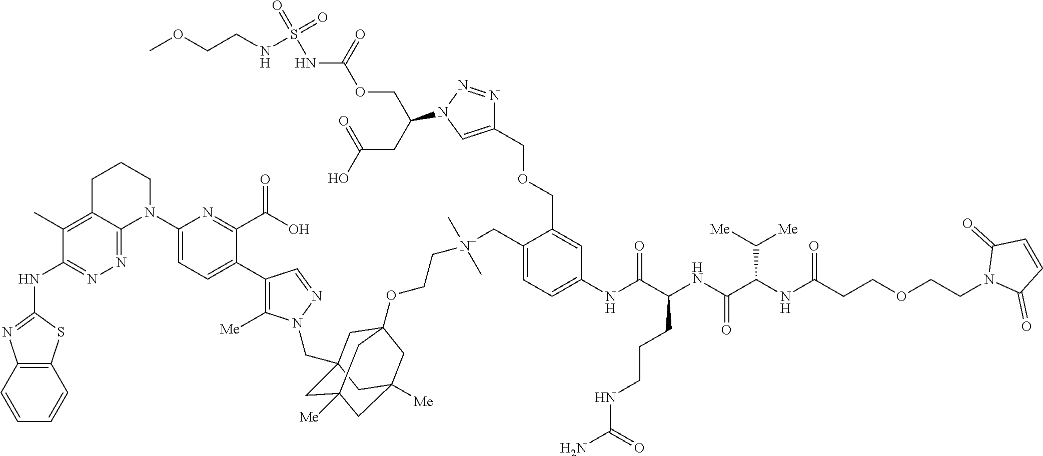 [SMILES: COCCNS(=O)(=O)NC(=O)OC[C@H](CC(=O)O)n1cc(COCc2cc(NC(=O)[C@H](CCCNC(N)=O)NC(=O)[C@@H](NC(=O)CCOCCN3C(=O)C=CC3=O)C(C)C)ccc2C[N+](C)(C)CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)nn1] |
| L103A-P1 | 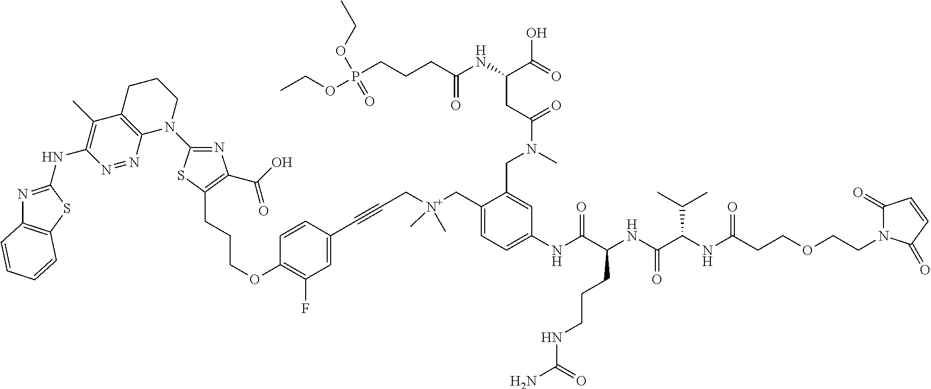 [SMILES: CCOP(=O)(CCCC(=O)N[C@@H](CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CC#Cc1ccc(OCCCc2sc(N3CCCc4c3nnc(Nc3nc5ccccc5s3)c4C)nc2C(=O)O)c(F)c1)C(=O)O)OCC] |
| L103C-P19 | 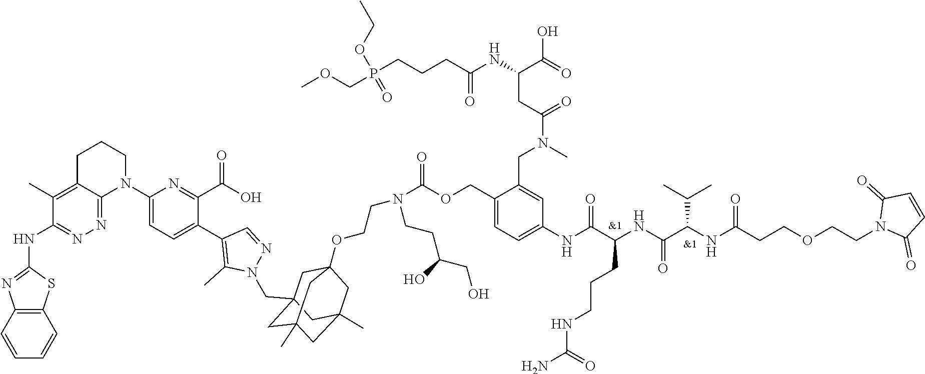 [SMILES: CCOP(=O)(CCCC(=O)N[C@@H](CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)CC[C@H](O)CO)C(=O)O)COC] |
| L103A-P21 | 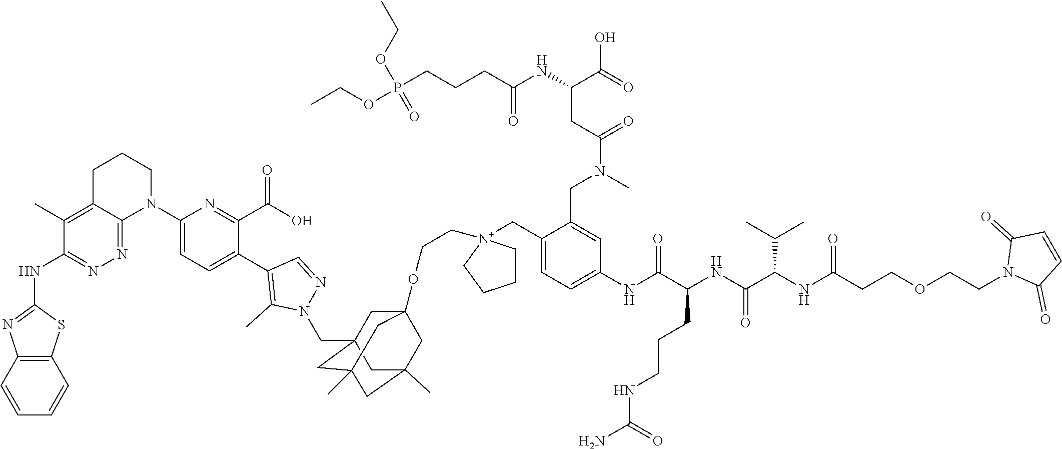 [SMILES: CCOP(=O)(CCCC(=O)N[C@@H](CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+]1(CCOC23CC4(C)CC(C)(CC(Cn5ncc(-c6ccc(N7CCCc8c7nnc(Nc7nc9ccccc9s7)c8C)nc6C(=O)O)c5C)(C4)C2)C3)CCCC1)C(=O)O)OCC] |
| L103C-P25 | 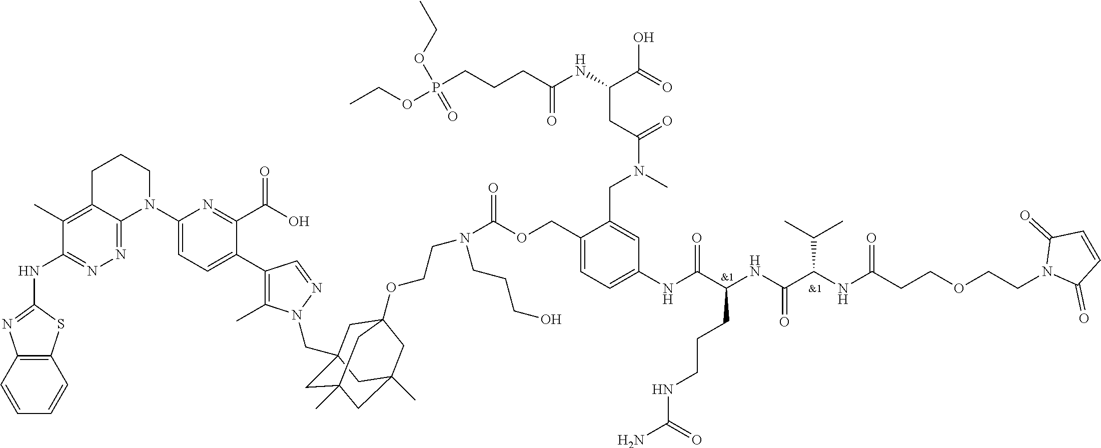 [SMILES: CCOP(=O)(CCCC(=O)N[C@@H](CC(=O)N(C)Cc1cc(NC(=O)[C@H](CCCNC(N)=O)NC(=O)[C@@H](NC(=O)CCOCCN2C(=O)C=CC2=O)C(C)C)ccc1COC(=O)N(CCCO)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)C(=O)O)OCC] |
| L103A-P27 | 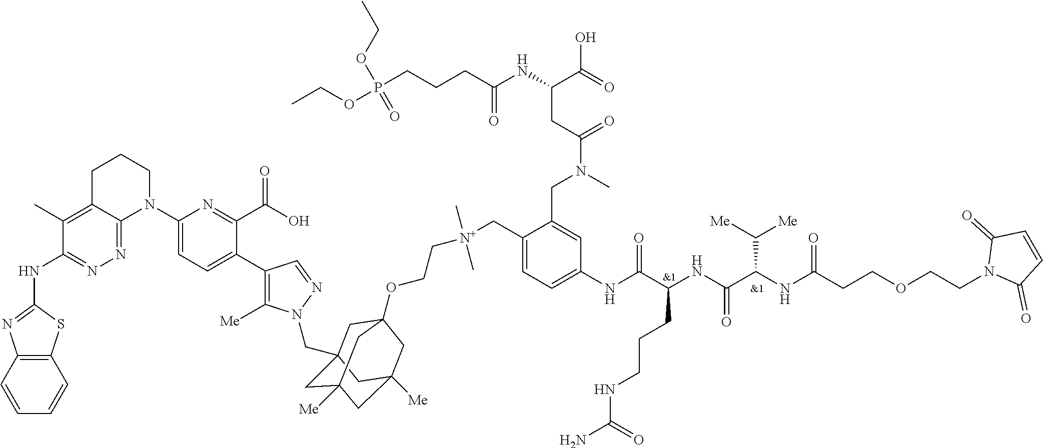 [SMILES: CCOP(=O)(CCCC(=O)N[C@@H](CC(=O)N(C)Cc1cc(NC(=O)[C@H](CCCNC(N)=O)NC(=O)[C@@H](NC(=O)CCOCCN2C(=O)C=CC2=O)C(C)C)ccc1C[N+](C)(C)CCOC12CC3(C)CC(C)(CC(Cn4ncc(-c5ccc(N6CCCc7c6nnc(Nc6nc8ccccc8s6)c7C)nc5C(=O)O)c4C)(C3)C1)C2)C(=O)O)OCC] |
| L106A-P2 | 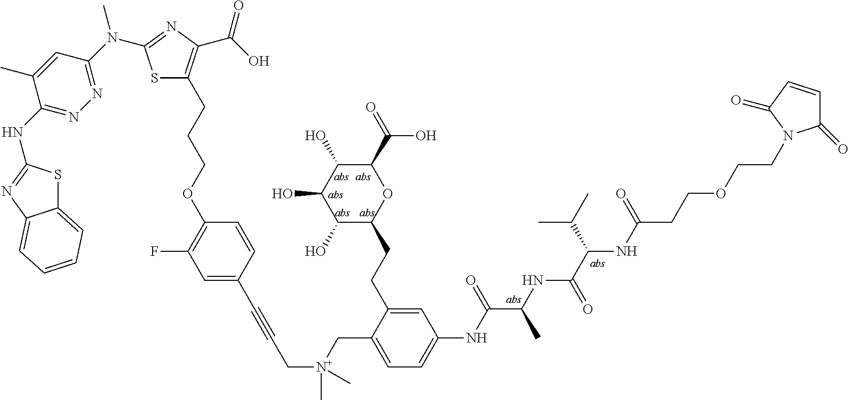 [SMILES: Cc1cc(N(C)c2nc(C(=O)O)c(CCCOc3ccc(C#CC[N+](C)(C)Cc4ccc(NC(=O)[C@H](C)NC(=O)[C@@H](NC(=O)CCOCCN5C(=O)C=CC5=O)C(C)C)cc4CC[C@@H]4O[C@H](C(=O)O)[C@@H](O)[C@H](O)[C@H]4O)cc3F)s2)nnc1Nc1nc2ccccc2s1] |
| L106C-P7 | 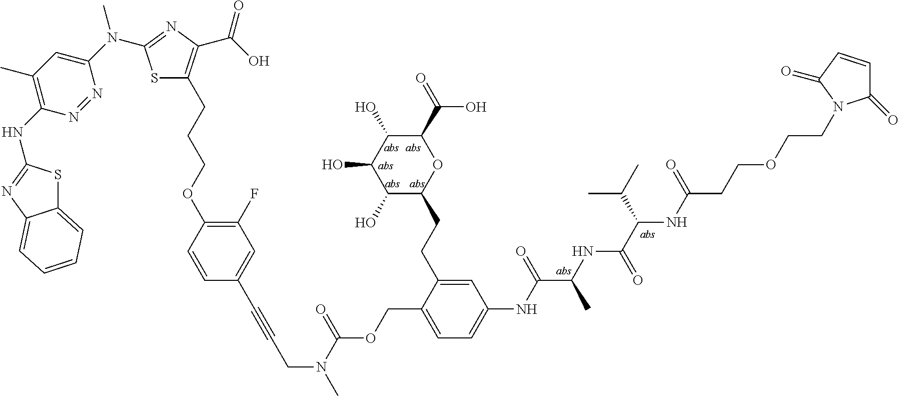 [SMILES: Cc1cc(N(C)c2nc(C(=O)O)c(CCCOc3ccc(C#CCN(C)C(=O)OCc4ccc(NC(=O)[C@H](C)NC(=O)[C@@H](NC(=O)CCOCCN5C(=O)C=CC5=O)C(C)C)cc4CC[C@@H]4O[C@H](C(=O)O)[C@@H](O)[C@H](O)[C@H]4O)cc3F)s2)nnc1Nc1nc2ccccc2s1] |
| L107C-P7 | 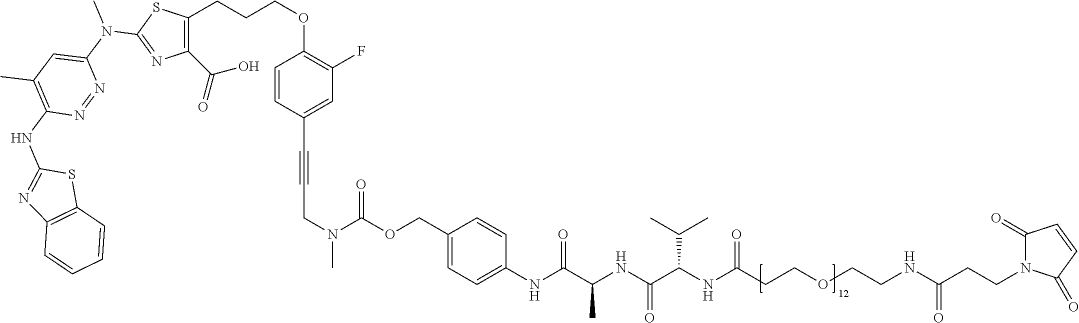 |
| L107A-P2 | 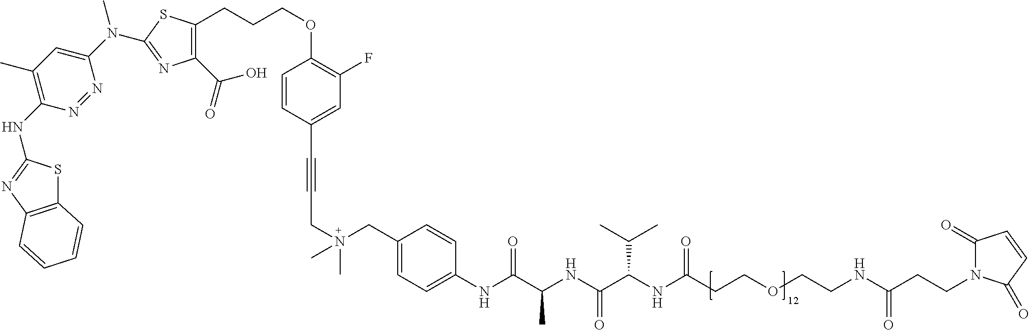 |
| L108A-P2 | 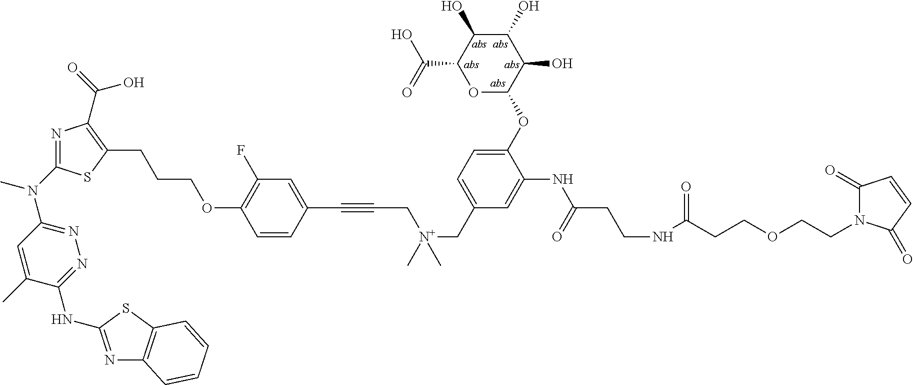 [SMILES: Cc1cc(N(C)c2nc(C(=O)O)c(CCCOc3ccc(C#CC[N+](C)(C)Cc4ccc(O[C@@H]5O[C@H](C(=O)O)[C@@H](O)[C@H](O)[C@H]5O)c(NC(=O)CCNC(=O)CCOCCN5C(=O)C=CC5=O)c4)cc3F)s2)nnc1Nc1nc2ccccc2s1] |
| L109A-P1 | 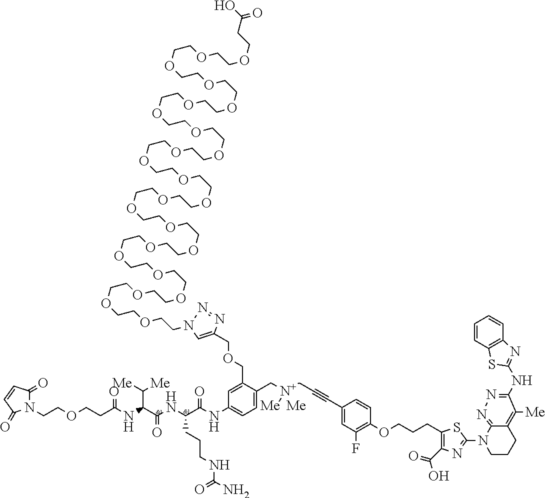 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3COCc3cn(CCOCCOCCOCCOCCOCCOCCOCCOCCOCCOCCOCCOCCOCCOCCOCCOCCOCCOCCOCCOCCOCCOCCOCCOCCC(=O)O)nn3)cc2F)s1] |
| L110C-P7 | 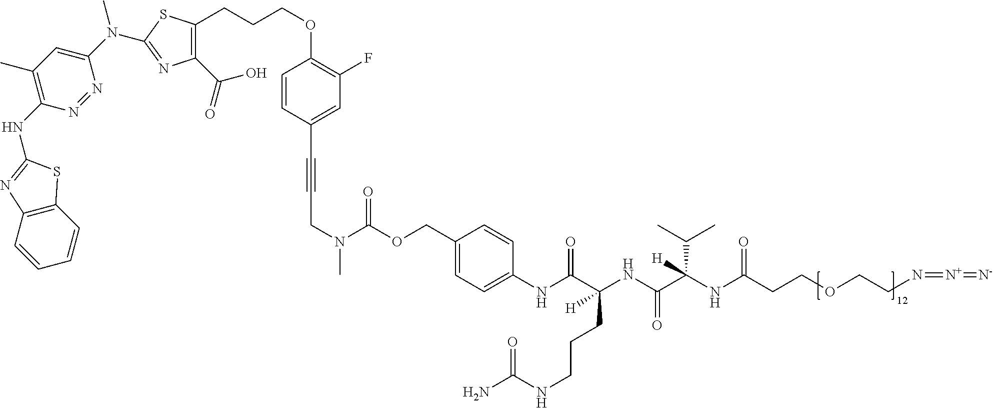 |
| L111A-P1 | 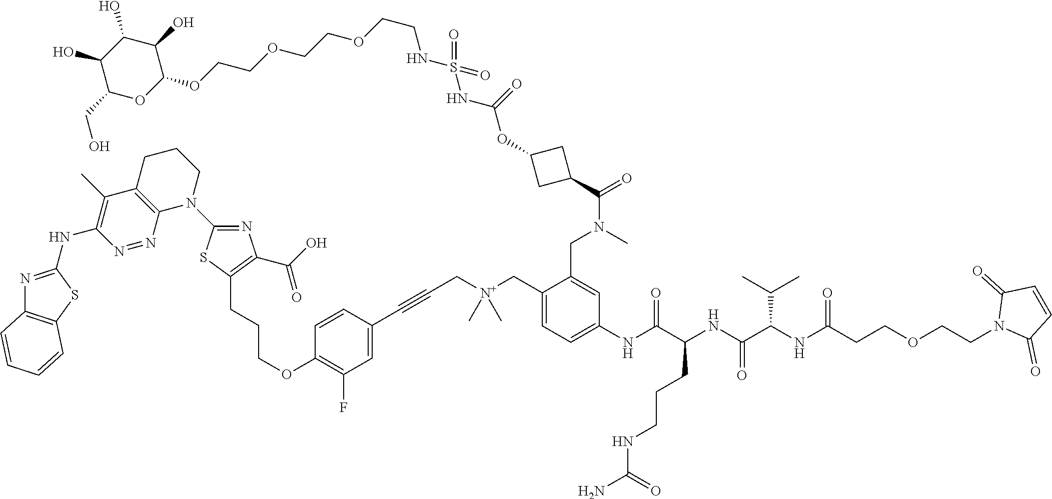 [SMILES: Cc1c(Nc2nc3ccccc3s2)nnc2c1CCCN2c1nc(C(=O)O)c(CCCOc2ccc(C#CC[N+](C)(C)Cc3ccc(NC(=O)[C@H](CCCNC(N)=O)NC(=O)[C@@H](NC(=O)CCOCCN4C(=O)C=CC4=O)C(C)C)cc3CN(C)C(=O)[C@H]3C[C@H](OC(=O)NS(=O)(=O)NCCOCCOCCO[C@@H]4O[C@H](CO)[C@@H](O)[C@H](O)[C@H]4O)C3)cc2F)s1] |
| L111C-P19 | 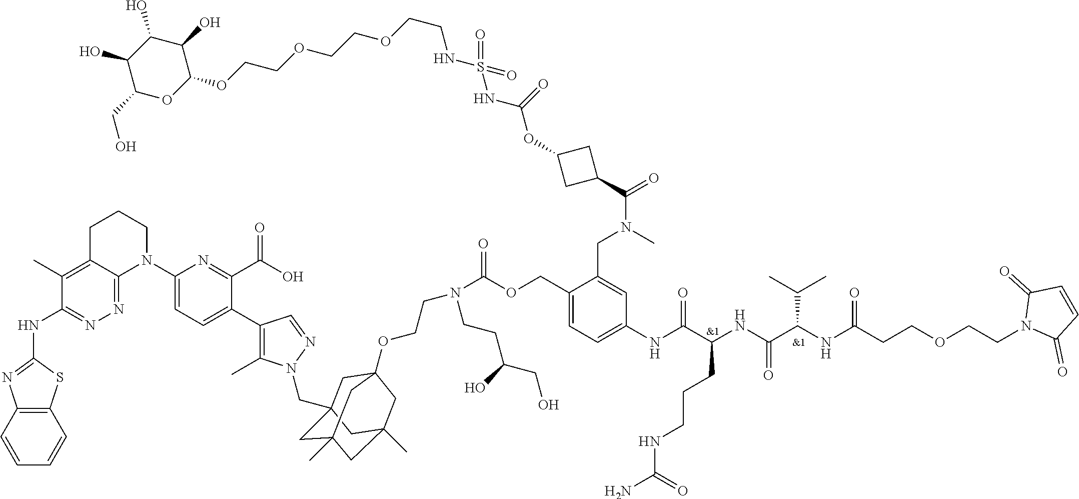 [SMILES: Cc1c(Nc2nc3ccccc3s2)nnc2c1CCCN2c1ccc(-c2cnn(CC34CC5(C)CC(C)(C3)CC(OCCN(CC[C@H](O)CO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)[C@H]3C[C@H](OC(=O)NS(=O)(=O)NCCOCCOCCO[C@@H]6O[C@H](CO)[C@@H](O)[C@H](O)[C@H]6O)C3)(C5)C4)c2C)c(C(=O)O)n1] |
| L111A-P21 | 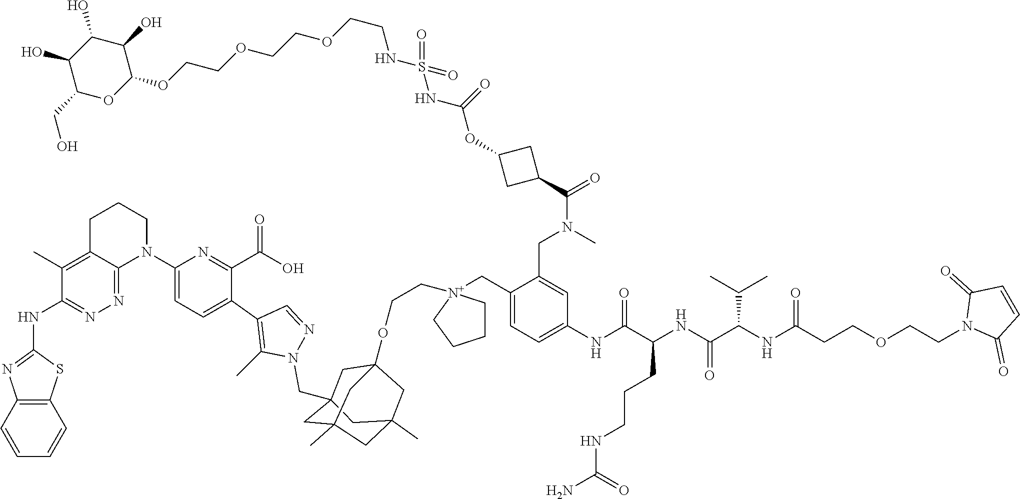 [SMILES: Cc1c(Nc2nc3ccccc3s2)nnc2c1CCCN2c1ccc(-c2cnn(CC34CC5(C)CC(C)(C3)CC(OCC[N+]3(Cc6ccc(NC(=O)[C@H](CCCNC(N)=O)NC(=O)[C@@H](NC(=O)CCOCCN7C(=O)C=CC7=O)C(C)C)cc6CN(C)C(=O)[C@H]6C[C@H](OC(=O)NS(=O)(=O)NCCOCCOCCO[C@@H]7O[C@H](CO)[C@@H](O)[C@H](O)[C@H]7O)C6)CCCC3)(C5)C4)c2C)c(C(=O)O)n1] |
| L111C-P25 | 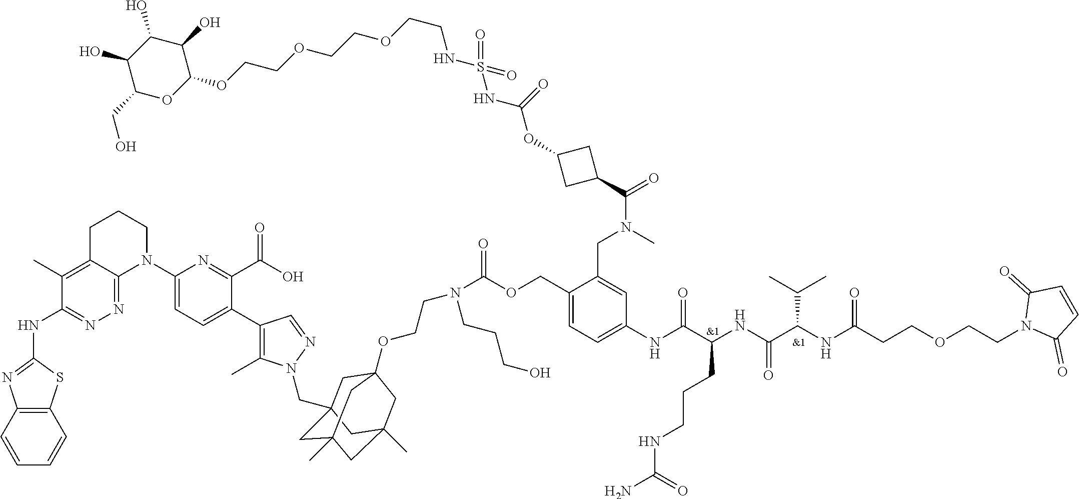 [SMILES: Cc1c(Nc2nc3ccccc3s2)nnc2c1CCCN2c1ccc(-c2cnn(CC34CC5(C)CC(C)(C3)CC(OCCN(CCCO)C(=O)OCc3ccc(NC(=O)[C@H](CCCNC(N)=O)NC(=O)[C@@H](NC(=O)CCOCCN6C(=O)C=CC6=O)C(C)C)cc3CN(C)C(=O)[C@H]3C[C@H](OC(=O)NS(=O)(=O)NCCOCCOCCO[C@@H]6O[C@H](CO)[C@@H](O)[C@H](O)[C@H]6O)C3)(C5)C4)c2C)c(C(=O)O)n1] |
| L111A-P27 | 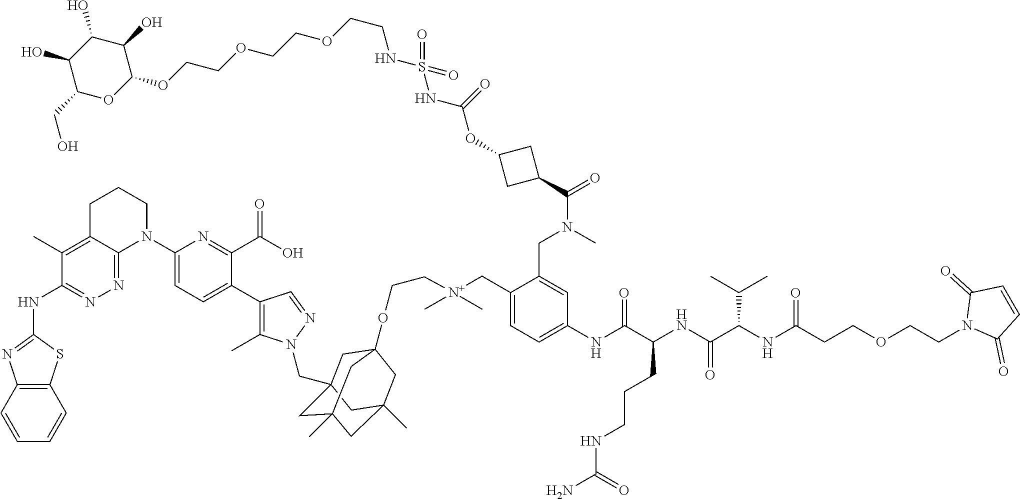 [SMILES: Cc1c(Nc2nc3ccccc3s2)nnc2c1CCCN2c1ccc(-c2cnn(CC34CC5(C)CC(C)(C3)CC(OCC[N+](C)(C)Cc3ccc(NC(=O)[C@H](CCCNC(N)=O)NC(=O)[C@@H](NC(=O)CCOCCN6C(=O)C=CC6=O)C(C)C)cc3CN(C)C(=O)[C@H]3C[C@H](OC(=O)NS(=O)(=O)NCCOCCOCCO[C@@H]6O[C@H](CO)[C@@H](O)[C@H](O)[C@H]6O)C3)(C5)C4)c2C)c(C(=O)O)n1] |
| L112A-P1 | 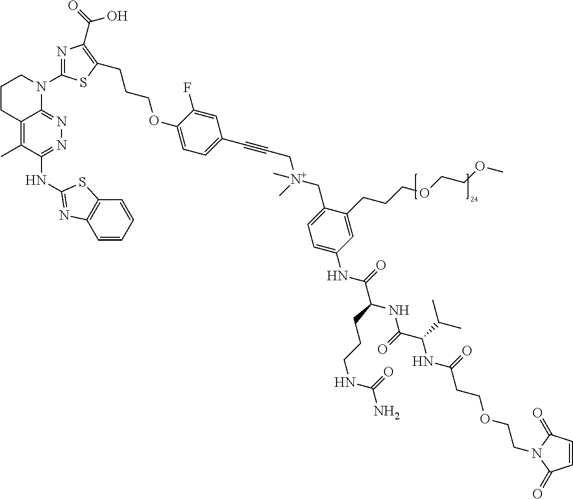 |

or an enantiomer, diastereoisomer, and/or pharmaceutically acceptable salt thereof.

58. The antibody-drug conjugate of claim 1, wherein:
(1) the anti-EphA2 antibody or antigen-binding fragment comprises a heavy chain variable region comprising an amino acid sequence of SEQ ID NO: 1, and a light chain variable region comprising an amino acid sequence of SEQ ID NO: 11, 19, 20, 21, 22, 23, 25, 28, 31, 34, 71, 72, or 73;
(2) the anti-EphA2 antibody or antigen-binding fragment comprises a heavy chain comprising an amino acid sequence of SEQ ID NO: 37, and a light chain comprising an amino acid sequence of SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77;
(3) the anti-EphA2 antibody or antigen-binding fragment comprises a heavy chain comprising an amino acid sequence of SEQ ID NO: 39, and a light chain comprising an amino acid sequence of SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77;
(4) the anti-EphA2 antibody or antigen-binding fragment comprises a heavy chain comprising an amino acid sequence of SEQ ID NO: 74, and a light chain comprising an amino acid sequence of SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 75, SEQ ID NO: 76, or SEQ ID NO: 77;
(5) the anti-EphA2 antibody or antigen-binding fragment comprises:
a heavy chain comprising an amino acid sequence of SEQ ID NO: 39, and a light chain comprising an amino acid sequence of SEQ ID NO: 41; or
(6) the anti-EphA2 antibody or antigen-binding fragment comprises:
a heavy chain comprising an amino acid sequence of SEQ ID NO: 74, and a light chain comprising an amino acid sequence of SEQ ID NO: 41.

59-63. (canceled)

64. A composition comprising multiple copies of the antibody-drug conjugate of claim 1, wherein the average p of the antibody-drug conjugates in the composition is from about 2 to about 16, e.g., about 2 to about 8, e.g., about 2 to about 4.

65. A pharmaceutical composition comprising the antibody-drug conjugate of claim 1, and a pharmaceutically acceptable carrier.

66. A method of (a) treating a subject having or suspected of having a cancer, (b) reducing or inhibiting the growth of a tumor in a subject, (c) reducing or inhibiting a hematological cancer in a subject, or (d) reducing or slowing the expansion of a cancer cell population in a subject, comprising administering to the subject a therapeutically effective amount of the antibody-drug conjugate of claim 1, the composition of claim 64, or the pharmaceutical composition of claim 65.

67-88. (canceled)

89. A method of inhibiting Bcl-xL activity in a cell that expresses Bcl-xL, comprising contacting the cell with an antibody-drug conjugate of claim 1 that is capable of binding the cell, under conditions in which the antibody drug conjugate binds the cell.

90. A method of determining whether a subject having or suspected of having a cancer will be responsive to treatment with the antibody-drug conjugate of claim 1, comprising providing a biological sample from the subject; contacting the sample with the antibody-drug conjugate; and detecting binding of the antibody-drug conjugate to cancer cells in the sample.

91-95. (canceled)

96. A method of producing the antibody-drug conjugate of claim 1, comprising reacting an anti-EphA2 antibody or antigen-binding fragment with a cleavable linker joined to a Bcl-xL inhibitor under conditions that allow conjugation.


SEQUENCE LISTING (78 sequences)

SEQ ID NO: 1 - protein, 128 aa, synthetic construct
  REGION 1..128: note = Synthetic
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSS

SEQ ID NO: 2 - protein, 7 aa, synthetic construct
  REGION 1..7: note = Synthetic
GFTFSHY

SEQ ID NO: 3 - protein, 6 aa, synthetic construct
  REGION 1..6: note = Synthetic
GPSGGP

SEQ ID NO: 4 - protein, 19 aa, synthetic construct
  REGION 1..19: note = Synthetic
YDSGYDYVAVAGPAEYFQH

SEQ ID NO: 5 - protein, 5 aa, synthetic construct
  REGION 1..5: note = Synthetic
HYMMA

SEQ ID NO: 6 - protein, 17 aa, synthetic construct
  REGION 1..17: note = Synthetic
RIGPSGGPTHYADSVKG

SEQ ID NO: 7 - protein, 8 aa, synthetic construct
  REGION 1..8: note = Synthetic
GFTFSHYM

SEQ ID NO: 8 - protein, 8 aa, synthetic construct
  REGION 1..8: note = Synthetic
IGPSGGPT

SEQ ID NO: 9 - protein, 21 aa, synthetic construct
  REGION 1..21: note = Synthetic
AGYDSGYDYVAVAGPAEYFQH

SEQ ID NO: 10 - protein, 10 aa, synthetic construct
  REGION 1..10: note = Synthetic
GFTFSHYMMA

SEQ ID NO: 11 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIK

SEQ ID NO: 12 - protein, 7 aa, synthetic construct
  REGION 1..7: note = Synthetic
SQSISTW

SEQ ID NO: 13 - skipped

SEQ ID NO: 14 - protein, 6 aa, synthetic construct
  REGION 1..6: note = Synthetic
YNSYSR

SEQ ID NO: 15 - protein, 11 aa, synthetic construct
  REGION 1..11: note = Synthetic
RASQSISTWLA

SEQ ID NO: 16 - protein, 7 aa, synthetic construct
  REGION 1..7: note = Synthetic
KASNLHT

SEQ ID NO: 17 - protein, 9 aa, synthetic construct
  REGION 1..9: note = Synthetic
QQYNSYSRT

SEQ ID NO: 18 - protein, 6 aa, synthetic construct
  REGION 1..6: note = Synthetic
QSISTW

SEQ ID NO: 19 - protein, 107 aa, synthetic construct
  REGION 1..107: note = Synthetic
DIQMTQSPSTLSASVGDRVTITCRASQSISTWLAWYQQKPGKAPKLLIYKASNLHTGVPSRFSGSGSGTEFSLTISGLQPDDFATYYCQQYNSYSRTFGQGTKVEIK

SEQ ID NO: 20 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFTLTISGLQPDDFATYYCQQYNSYSRTFGQGTKVEIK

SEQ ID NO: 21 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFSLTISSLQPDDFATYYCQQYNSYSRTFGQGTKVEIK

SEQ ID NO: 22 - protein, 107 aa, synthetic construct
  REGION 1..107: note = Synthetic
DIQMTQSPSTLSASVGDRVTITCRASQSISTWLAWYQQKPGKAPKLLIYKASNLHTGVPSRFSGSGSGTEFTLTISSLQPDDFATYYCQQYNSYSRTFGQGTKVEIK

SEQ ID NO: 23 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYNQYSRTFGQGTKVEIK

SEQ ID NO: 24 - protein, 9 aa, synthetic construct
  REGION 1..9: note = Synthetic
QQYNQYSRT

SEQ ID NO: 25 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYNVYSRTFGQGTKVEIK

SEQ ID NO: 26 - protein, 6 aa, synthetic construct
  REGION 1..6: note = Synthetic
YNVYSR

SEQ ID NO: 27 - protein, 9 aa, synthetic construct
  REGION 1..9: note = Synthetic
QQYNVYSRT

SEQ ID NO: 28 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYNAYSRTFGQGTKVEIK

SEQ ID NO: 29 - protein, 6 aa, synthetic construct
  REGION 1..6: note = Synthetic
YNAYSR

SEQ ID NO: 30 - protein, 9 aa, synthetic construct
  REGION 1..9: note = Synthetic
QQYNAYSRT

SEQ ID NO: 31 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYASYSRTFGQGTKVEIK

SEQ ID NO: 32 - protein, 6 aa, synthetic construct
  REGION 1..6: note = Synthetic
YASYSR

SEQ ID NO: 33 - protein, 9 aa, synthetic construct
  REGION 1..9: note = Synthetic
QQYASYSRT

SEQ ID NO: 34 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYQSYSRTFGQGTKVEIK

SEQ ID NO: 35 - protein, 6 aa, synthetic construct
  REGION 1..6: note = Synthetic
YQSYSR

SEQ ID NO: 36 - protein, 9 aa, synthetic construct
  REGION 1..9: note = Synthetic
QQYQSYSRT

SEQ ID NO: 37 - protein, 458 aa, synthetic construct
  REGION 1..458: note = Synthetic
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 38 - DNA, 1374 bp, synthetic construct
  misc_feature 1..1374: note = Synthetic
  source 1..1374: mol_type = other DNA
gaagttcagctgcttgaatctggcggcggactggttcaacctggcggatctctgagactgagctgtgccgccagcggcttcaccttcagccactatatgatggcctgggtccgacaggcccctggcaaaggacttgaatgggtgtccagaatcggcccctctggcggccctacacactacgctgattctgtgaagggcagattcaccatcagccgggacaacagcaagaacaccctgtacctgcagatgaacagcctgagagccgaggacaccgccgtgtattactgtgccggctacgacagcggctacgattatgtggctgtggccggacctgccgagtactttcagcattggggacagggcaccctggtcaccgttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 39 - protein, 458 aa, synthetic construct
  REGION 1..458: note = Synthetic
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 40 - DNA, 1374 bp, synthetic construct
  misc_feature 1..1374: note = Synthetic
  source 1..1374: mol_type = other DNA
gaagttcagctgcttgaatctggcggcggactggttcaacctggcggatctctgagactgagctgtgccgccagcggcttcaccttcagccactatatgatggcctgggtccgacaggcccctggcaaaggacttgaatgggtgtccagaatcggcccctctggcggccctacacactacgctgattctgtgaagggcagattcaccatcagccgggacaacagcaagaacaccctgtacctgcagatgaacagcctgagagccgaggacaccgccgtgtattactgtgccggctacgacagcggctacgattatgtggctgtggccggacctgccgagtactttcagcattggggacagggcaccctggtcaccgttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggccgtgtcccacgaggacccagaggtgaagttcaactggtacgtggacggcgtggaggtgcacaacgccaagaccaagcccagagaggagcagtacgccagcacctacagggtggtgtccgtgctgaccgtgctgcaccaggactggctgaacggcaaagaatacaagtgcaaagtctccaacaaggccctggctgccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 41 - protein, 214 aa, synthetic construct
  REGION 1..214: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - DNA, 642 bp, synthetic construct
  misc_feature 1..642: note = Synthetic
  source 1..642: mol_type = other DNA
gacattcagatgacacagagccctagcagcctgagcgcctctgtgggagacagagtgaccatcacctgtagagccagccagagcatcagcacatggctggcctggtatcagcagaagcctggcaaggcccctaagctgctgatctacaaggccagcaatctgcacaccggcgtgcccagcagattttctggctctggaagcggcaccgagttcagcctgacaatctctggcctgcagcctgacgacttcgccacctactactgccagcagtacaacagctacagccggacctttggccagggaacaaaggtggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 43 - protein, 214 aa, synthetic construct
  REGION 1..214: note = Synthetic
DIQMTQSPSTLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 640 bp, synthetic construct
  misc_feature 1..640: note = Synthetic
  source 1..640: mol_type = other DNA
acattcagatgacacagagccctagcacactgagcgccagcgtgggagacagagtgaccatcacatgtagagccagccagagcatcagcacctggctggcatggtatcagcagaagcctggcaaggcccctaagctgctgatctacaaggccagcaatctgcacaccggcgtgcccagcagattttctggctctggaagcggcaccgagttcagcctgacaatctctggcctgcagcctgacgacttcgccacctactactgccagcagtacaacagctacagccggacctttggccagggaacaaaggtggaaatcaagcgtacgtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 45 - protein, 214 aa, synthetic construct
  REGION 1..214: note = Synthetic
DIQMTQSPSSLSASVGDRVTITCRASQSISTWLAWYQQKPGKAPKLLIYKASNLHTGVPSRFSGSGSGTEFTLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 641 bp, synthetic construct
  misc_feature 1..641: note = Synthetic
  source 1..641: mol_type = other DNA
gacattcagatgacacagagccctagcagcctgagcgcctctgtgggagacagagtgaccatcacctgtagagccagccagagcatcagcacatggctggcctggtatcagcagaagcctggcaaggcccctaagctgctgatctacaaggccagcaatctgcacaccggcgtgcccagcagattttctggctctggaagcggcaccgagttcaccctgacaatctctggcctgcagcctgacgacttcgccacctactactgccagcagtacaacagctacagccggacctttggccagggaacaaaggtggaaatcaagcgtacgtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 47 - protein, 214 aa, synthetic construct
  REGION 1..214: note = Synthetic
DIQMTQSPSSLSASVGDRVTITCRASQSISTWLAWYQQKPGKAPKLLIYKASNLHTGVPSRFSGSGSGTEFSLTISSLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 641 bp, synthetic construct
  misc_feature 1..641: note = Synthetic
  source 1..641: mol_type = other DNA
gacattcagatgacacagagccctagcagcctgagcgcctctgtgggagacagagtgaccatcacctgtagagccagccagagcatcagcacatggctggcctggtatcagcagaagcctggcaaggcccctaagctgctgatctacaaggccagcaatctgcacaccggcgtgcccagcagattttctggctctggaagcggcaccgagttcagcctgacaatcagcagcctgcagcctgacgacttcgccacctactactgccagcagtacaacagctacagccggacctttggccagggaacaaaggtggaaatcaagcgtacgtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 49 - protein, 214 aa, synthetic construct
  REGION 1..214: note = Synthetic
DIQMTQSPSTLSASVGDRVTITCRASQSISTWLAWYQQKPGKAPKLLIYKASNLHTGVPSRFSGSGSGTEFTLTISSLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - DNA, 641 bp, synthetic construct
  misc_feature 1..641: note = Synthetic
  source 1..641: mol_type = other DNA
gacattcagatgacacagagccctagcacactgagcgccagcgtgggagacagagtgaccatcacatgtagagccagccagagcatcagcacctggctggcatggtatcagcagaagcctggcaaggcccctaagctgctgatctacaaggccagcaatctgcacaccggcgtgcccagcagattttctggctctggaagcggcaccgagttcaccctgaccatcagtagcctgcagcctgacgacttcgccacctactactgccagcagtacaacagctacagccggacctttggccagggaacaaaggtggaaatcaagcgtacgtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 51 - protein, 214 aa, synthetic construct
  REGION 1..214: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYNQYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - DNA, 641 bp, synthetic construct
  misc_feature 1..641: note = Synthetic
  source 1..641: mol_type = other DNA
gacattcagatgacacagagccctagcagcctgagcgcctctgtgggagacagagtgaccatcacctgtagagccagccagagcatcagcacatggctggcctggtatcagcagaagcctggcaaggcccctaagctgctgatctacaaggccagcaatctgcacaccggcgtgcccagcagattttctggctctggaagcggcaccgagttcagcctgacaatctctggcctgcagcctgacgacttcgccacctactactgccagcagtacaaccagtacagccggacctttggccagggaacaaaggtggaaatcaagcgtacgtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 53 - protein, 214 aa, synthetic construct
  REGION 1..214: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYNVYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - DNA, 641 bp, synthetic construct
  misc_feature 1..641: note = Synthetic
  source 1..641: mol_type = other DNA
gacattcagatgacacagagccctagcagcctgagcgcctctgtgggagacagagtgaccatcacctgtagagccagccagagcatcagcacatggctggcctggtatcagcagaagcctggcaaggcccctaagctgctgatctacaaggccagcaatctgcacaccggcgtgcccagcagattttctggctctggaagcggcaccgagttcagcctgacaatctctggcctgcagcctgacgacttcgccacctactactgccagcagtacaacgtgtacagccggacctttggccagggaacaaaggtggaaatcaagcgtacgtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 55 - protein, 214 aa, synthetic construct
  REGION 1..214: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYNAYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - DNA, 641 bp, synthetic construct
  misc_feature 1..641: note = Synthetic
  source 1..641: mol_type = other DNA
gacattcagatgacacagagccctagcagcctgagcgcctctgtgggagacagagtgaccatcacctgtagagccagccagagcatcagcacatggctggcctggtatcagcagaagcctggcaaggcccctaagctgctgatctacaaggccagcaatctgcacaccggcgtgcccagcagattttctggctctggaagcggcaccgagttcagcctgacaatctctggcctgcagcctgacgacttcgccacctactactgccagcagtacaacgcctacagccggacatttggccagggaacaaaggtggaaatcaagcgtacgtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 57 - protein, 321 aa, synthetic construct
  REGION 1..321: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYASYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - DNA, 641 bp, synthetic construct
  misc_feature 1..641: note = Synthetic
  source 1..641: mol_type = other DNA
gacattcagatgacacagagccctagcagcctgagcgcctctgtgggagacagagtgaccatcacctgtagagccagccagagcatcagcacatggctggcctggtatcagcagaagcctggcaaggcccctaagctgctgatctacaaggccagcaatctgcacaccggcgtgcccagcagattttctggctctggaagcggcaccgagttcagcctgacaatctctggcctgcagcctgacgacttcgccacctactactgtcagcagtacgccagctacagccggacatttggccagggaacaaaggtggaaatcaagcgtacgtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 59 - protein, 214 aa, synthetic construct
  REGION 1..214: note = Synthetic
DIQMTQSPSSLSASVGDRVTITCRASQSISTWLAWYQQKPGKAPKLLIYKASNLHTGVPSRFSGSGSGTEFSLTISGLQPDDFATYYCQQYQSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - DNA, 641 bp, synthetic construct
  misc_feature 1..641: note = Synthetic
  source 1..641: mol_type = other DNA
gacattcagatgacacagagccctagcagcctgagcgcctctgtgggagacagagtgaccatcacctgtagagccagccagagcatcagcacatggctggcctggtatcagcagaagcctggcaaggcccctaagctgctgatctacaaggccagcaatctgcacaccggcgtgcccagcagattttctggctctggaagcggcaccgagttcagcctgacaatctctggcctgcagcctgacgacttcgccacctactactgccagcagtaccagagctacagccggacatttggccagggaacaaaggtggaaatcaagcgtacgtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 61 - protein, 976 aa, synthetic construct
  REGION 1..976: note = Synthetic
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 62 - protein, 128 aa, synthetic construct
  REGION 1..128: note = Synthetic
QVQLQQSGPGLVKPSQTLSLTCAISGDSVSSNSAAWSWIRQSPGRGLEWLGRIYYRSKWYNDYAVSVKSRITINPDTSKNQFSLQLNSVTPEDTAVYYCARLDHRYHEDTVYPGMDVWGQGTLVTVSS

SEQ ID NO: 63 - protein, 12 aa, synthetic construct
  REGION 1..12: note = Synthetic
GDSVSSNSAAWS

SEQ ID NO: 64 - protein, 18 aa, synthetic construct
  REGION 1..18: note = Synthetic
RIYYRSKWYNDYAVSVKS

SEQ ID NO: 65 - protein, 16 aa, synthetic construct
  REGION 1..16: note = Synthetic
LDHRYHEDTVYPGMDV

SEQ ID NO: 66 - protein, 107 aa, synthetic construct
  REGION 1..107: note = Synthetic
DIELTQPPSVSVAPGQTARISCSGDNLPAYTVTWYQQKPGQAPVLVIYDDSDRPSGIPERFSGSNSGNTATLTISGTQAEDEADYYCASWDPSSGVVFGGGTKLTVL

SEQ ID NO: 67 - protein, 11 aa, synthetic construct
  REGION 1..11: note = Synthetic
SGDNLPAYTVT

SEQ ID NO: 68 - protein, 7 aa, synthetic construct
  REGION 1..7: note = Synthetic
DDSDRPS

SEQ ID NO: 69 - protein, 10 aa, synthetic construct
  REGION 1..10: note = Synthetic
ASWDPSSGVV

SEQ ID NO: 70 - protein, 6 aa, synthetic construct
  REGION 1..6: note = Synthetic
YNQYSR

SEQ ID NO: 71 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFTLTISGLQPDDFATYYCQQYQSYSRTFGQGTKVEIK

SEQ ID NO: 72 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFSLTISSLQPDDFATYYCQQYQSYSRTFGQGTKVEIK

SEQ ID NO: 73 - protein, 107 aa, synthetic construct
  REGION 1..107: note = Synthetic
DIQMTQSPSSLSASVGDRVTITCRASQSISTWLAWYQQKPGKAPKLLIYKASNLHTGVPSRFSGSGSGTEFTLTISSLQPDDFATYYCQQYQSYSRTFGQGTKVEIK

SEQ ID NO: 74 - protein, 458 aa, synthetic construct
  REGION 1..458: note = Synthetic
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 214 aa, synthetic construct
  REGION 1..214: note = Synthetic
DIQMTQSPSSLSASVGDRVTITCRASQSISTWLAWYQQKPGKAPKLLIYKASNLHTGVPSRFSGSGSGTEFTLTISGLQPDDFATYYCQQYQSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 214 aa, synthetic construct
  REGION 1..214: note = Synthetic
DIQMTQSPSSLSASVGDRVTITCRASQSISTWLAWYQQKPGKAPKLLIYKASNLHTGVPSRFSGSGSGTEFSLTISSLQPDDFATYYCQQYQSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 214 aa, synthetic construct
  REGION 1..214: note = Synthetic
DIQMTQSPSSLSASVGDRVTITCRASQSISTWLAWYQQKPGKAPKLLIYKASNLHTGVPSRFSGSGSGTEFTLTISSLQPDDFATYYCQQYQSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 4 aa, synthetic construct
  REGION 1..4: note = Synthetic
GGFG